SEQUENCE LISTING (23 sequences)

SEQ ID NO: 1 - RNA, 21 nt, Artificial Sequence
  PFKFB3
gucuucggugucuccauuaau

SEQ ID NO: 2 - RNA, 21 nt, Artificial Sequence
  PFKFB3
gaagcaguacagcuccuacaa

SEQ ID NO: 3 - RNA, 21 nt, Artificial Sequence
  PFKFB3
gcaguacagcuccuacaacuu

SEQ ID NO: 4 - RNA, 21 nt, Artificial Sequence
  PFKFB3
gccuuagcugccuugagagau

SEQ ID NO: 5 - RNA, 21 nt, Artificial Sequence
  PFKFB3
gaagaggaucaguugcuauga

SEQ ID NO: 6 - RNA, 21 nt, Artificial Sequence
  PFKFB3
gacaccuacccugaggaguau

SEQ ID NO: 7 - RNA, 21 nt, Artificial Sequence
  PFKFB3
gggagcaggacaaguacuauu

SEQ ID NO: 8 - RNA, 21 nt, Artificial Sequence
  PFKFB3
ggagcaggacaaguacuauua

SEQ ID NO: 9 - RNA, 21 nt, Artificial Sequence
  PFKFB3
gcaggagaaugugcuggucau

SEQ ID NO: 10 - RNA, 21 nt, Artificial Sequence
  PFKFB3
gccuacuuccuggauaagagu

SEQ ID NO: 11 - RNA, 21 nt, Artificial Sequence
  PFKFB3
ggauaagagugcagaggagau

SEQ ID NO: 12 - RNA, 21 nt, Artificial Sequence
  PFKFB3
gaggucagaggaugcaaagaa

SEQ ID NO: 13 - RNA, 21 nt, Artificial Sequence
  PFKFB3
ggaugcaaagaagggaccuaa

SEQ ID NO: 14 - DNA, 23 bp, Artificial Sequence
  PFKFB3
aatgcgacaggtgattcccgtgg

SEQ ID NO: 15 - DNA, 23 bp, Artificial Sequence
  PFKFB3
ttaccgctaccccaccggggagg

SEQ ID NO: 16 - DNA, 23 bp, Artificial Sequence
  PFKFB3
agctacctggcgaaagaaggggg

SEQ ID NO: 17 - DNA, 23 bp, Artificial Sequence
  PFKFB3
tcgacgcggtgagtcctgggagg

SEQ ID NO: 18 - DNA, 23 bp, Artificial Sequence
  PFKFB3
aggtaggagtcccggtgacgcgg

SEQ ID NO: 19 - DNA, 23 bp, Artificial Sequence
  PFKFB3
caggtacctcgggcaggtcgtgg

SEQ ID NO: 20 - DNA, 23 bp, Artificial Sequence
  PFKFB3
tttcctgaagggcatcgcgccgg

SEQ ID NO: 21 - DNA, 23 bp, Artificial Sequence
  PFKFB3
accctgaggagtatgcgctgcgg

SEQ ID NO: 22 - RNA, 23 nt, Artificial Sequence
  PFKFB3
ggcaagcaggcagcgcaggacgg

SEQ ID NO: 23 - DNA, 23 bp, Artificial Sequence
  PFKFB3
ggcgctcaatgagatcgacgcgg

(19) United States
(12) Patent Application Publication
(10) Pub. No.: US 2023/0047816 A1
(43) Pub. Date: Feb. 16, 2023

(54) PFKFB3 INHIBITORS AND THEIR USES

(71) Applicants: Petr Olegovich FEDICHEV, Dolgoprudny (RU); Kevin GREENMAN, Sunnyvale, CA (US); Chang CHIH-TSUNG, East Palo Alto, CA (US); Maksim Nikolaevich KHOLIN, Moscow (RU); Evgeny Gennadievich GETMANTSEV, Moscow (RU); Tatiana Vladimirovna ZHIDKOVA, Moscow (RU); Alexandr Viktorovich KADUSHKIN, Moskovskaya (RU); Timofey Vladimirovich PYRKOV, Moscow (RU); Dmitry Veniaminovich SHISHOV, Verkhovashie (RU); Juan Pedro BOLANOS-HERNANDEZ, Salamanca (ES); Kristina Aleksandrovna ZAKURDAEVA, Moscow (RU); Olga Andreevna BURMISTROVA, Reutov (RU)

(72) Inventors: Petr Olegovich FEDICHEV, Dolgoprudny (RU); Kevin GREENMAN, Sunnyvale, CA (US); Chang CHIH-TSUNG, East Palo Alto, CA (US); Maksim Nikolaevich KHOLIN, Moscow (RU); Evgeny Gennadievich GETMANTSEV, Moscow (RU); Tatiana Vladimirovna ZHIDKOVA, Moscow (RU); Alexandr Viktorovich KADUSHKIN, Moskovskaya (RU); Timofey Vladimirovich PYRKOV, Moscow (RU); Dmitry Veniaminovich SHISHOV, Verkhovashie (RU); Juan Pedro BOLANOS-HERNANDEZ, Salamanca (ES); Kristina Aleksandrovna ZAKURDAEVA, Moscow (RU); Olga Andreevna BURMISTROVA, Reutov (RU)

(21) Appl. No.: 17/230,726

(22) Filed: Apr. 14, 2021

Related U.S. Application Data
(63) Continuation-in-part of application No. PCT/RU2019/095001, filed on Oct. 15, 2019.

(30) Foreign Application Priority Data
Oct. 15, 2018 (RU) 2018136333

(51) Int. Cl. C07D 401/14; C07D 209/48; C07D 403/10; C07D 409/04; C07D 409/14; C07D 403/04; C07D 403/14; C07D 405/14; C07D 209/46

(52) U.S. Cl. CPC C07D 401/14; C07D 209/48; C07D 403/10; C07D 409/04; C07B 2200/05; C07D 403/04; C07D 403/14; C07D 405/14; C07D 209/46; C07D 409/14

(57) ABSTRACT

This disclosure relates to new phthalimide and isoindolinone derivatives and other PFKFB3 inhibitors for use in the treatment of diseases. The invention further relates to pharmaceutical compositions containing such PFKFB3 inhibitors, methods of preparation thereof, methods for their use as therapeutic agents, and methods of preparation of a medicament for use in therapy, as well as kits and other inventions comprising such PFKFB3 inhibitors. These PFKFB3 inhibitors are useful for the treatment and prophylaxis of cancer, neurodegenerative diseases, autoimmune diseases, inflammatory disorders, multiple sclerosis, metabolic diseases, inhibition of angiogenesis and other diseases and conditions, where the modulation of PFKFB3 and/or PFKFB4 has beneficial effect as well as neuroprotection.

### CROSS-REFERENCE TO RELATED APPLICATIONS

This application is a continuation-in-part (CIP) of PCT Application No. PCT/RU2019/095001 filed on Oct. 15, 2019, which claims priority to Russian Patent Application No. RU2018136333 filed on Oct. 15, 2018, the contents of which are incorporated herein by reference in their entireties.

### SEQUENCE LISTING

The present application is being filed along with a Sequence Listing in electronic format. The Sequence Listing is provided as a file entitled 05_GERONZ00300_20210820_sequence_listing.txt, created Jun. 25, 2021, which is 4.27 Kb in size. The information in the electronic format of the Sequence Listing is incorporated herein by reference in its entirety.

### BACKGROUND OF THE INVENTION

Cancer cells exhibit preference for glycolysis instead of oxidative phosphorylation even in normoxia. Cancer cells benefit from elevated glycolytic flux to meet their high energy demands for rapid growth and proliferation. This finding is exploited clinically as a diagnostic tool for solid tumors, measuring the uptake of 2-Deoxy-2-[¹⁸F]fluoroglucose by positron-emission tomography (PET) imaging. In recent years glycolysis has drawn a revived attention due to its relation to cancer and the enzymes of glycolytic pathway were explored as potential targets for therapeutic intervention. Small-molecule inhibitors have been identified, for example, against glucose transporters, glyceraldehyde 3-phosphate dehydrogenase (GAPDH), hexokinase II, and hypoxia-inducible factor 1-alpha (HIF1-alpha). Inhibition of glycolysis (for example by 2-deoxy-D-glucose, bromopyruvic acid, Lonidamine, Phloretin, WZB1117) has been shown to promote cell death. However, directly targeting glycolytic enzymes may have detrimental effects to cells as evidenced by preclinical trials of Lonidamine, which revealed significant pancreatic and hepatic toxicities, and clinical trial of 2-deoxy-D-glucose, administration of which was related to hyperglycemia in all treated patients.

In this view, the regulatory role of 6-phosphofructo-2-kinase/fructose-2,6-biphosphatase 3 (PFKFB3) and 6-phosphofructo-2-kinase/fructose-2,6-biphosphatase 4 (PFKFB4) in cell metabolism and proliferation is of particular interest. Fructose-2,6-bisphosphate (Fru-2,6-BP) is a potent positive allosteric regulator of a key glycolytic enzyme phosphofructokinase-1 (PFK1). Cellular level of Fru-2,6-BP is dynamically regulated by the family of bifunctional enzymes 6-phosphofructo-2-kinase/fructose-2,6-bisphosphatases (PFKFB1-4), also referred to as phosphofructokinase-2 (PFK2). Increased level of Fru-2,6-BP promotes glycolytic flux by relieving the inhibitory effect of high ATP concentrations of PFK1.

Antisense siRNAs against PFKFB3 have been shown to inhibit cancer cell proliferation in vitro. Also, a decreased anchorage independent growth was observed for siRNA treated fibroblasts suggesting a potential antimetastatic effect. Recently, a key role of PFKFB3-driven glycolysis in vessel sprouting was identified. Silencing of PFKFB3 impaired endothelial tip cell activity also suggests an antiangiogenesis potential for a PFKFB3-targeting therapy.

PFKFB4 has shown to play a similar role in glycolytic flux but has different tissue distribution and has lower kinase:bisphosphatase ratio 4:1, as compared to 740:1 for PFKFB3. Both PFKFB3 and PFKFB4 are induced by hypoxia in various tumors. Interestingly, expression of PFKFB4 is higher than PFKFB3 in primary glioblastomas when compared with secondary glioblastomas as well as with the lower-grade astrocytomas and correlates with poor survival. PFKFB4 was shown to be important for glioma and prostate cancer cell survival.

PFKFB3 level and, consequently, Fru-2,6-BP and glycolysis are temporarily controlled during cell cycle progression and are elevated in G1-S phase transition. Another indication of the role of PFKFB3 in the cell cycle is the inactivation of cell-cycle inhibitor p27 and activation of cell-cycle promoting kinase Cdk1 by Fru-2,6-BP in the nucleus. These findings suggest that pharmacological inhibition of PFKFB3 kinase activity may go beyond solely regulating glycolysis metabolic flux. PFKFB3 is a key regulator of glycolysis, the central pathway of carbohydrate utilization, and as such is involved in several other disorders and pathological conditions. The proinflammatory cytokine interleukin-6 (IL6) was shown to enhance glycolytic flux in mouse embryonic fibroblasts and human cell lines. In vitro studies revealed that T-cell activation was accompanied by a marked increase of PFKFB3 level and Fru-2,6-BP concentration. Rheumatoid arthritis (RA) synovium is characterized by hypoxia induced changes in the expression of PFKFB3 and PFKFB4. Together these findings suggest a potential role of PFKFB3 inhibition for treatment of inflammatory conditions, autoimmune disorders, as well as application in immunosuppression.

Neurodegenerative pathologies are characterized by progressive loss of hippocampal and cortex neurons in Alzheimer's disease, dopaminergic neurons of the substantia nigra in Parkinson's disease, or motor neurons in Amyotrophic Lateral Sclerosis. Experimental data suggest that excitotoxicity along with mitochondrial dysfunction and increased ROS level are a common contributing cause. In normal conditions neurons maintain low level of PFKFB3, which is continuously degraded by the E3 ubiquitin ligase anaphase-promoting complex/cyclosome-Cdh1 (APC/C-Cdh1). During excitotoxicity, APC/C-Cdh1 is inhibited resulting in stabilization of PFKFB3, which leads to shifted glucose consumption by glycolysis at the expense of the pentose-phosphate pathway (PPP). This is detrimental to the redox status of glutathione and, hence, compromises the ability of neurons to detoxify reactive oxygen species (ROS) leading to apoptotic death.

A few small molecules have been postulated to inhibit kinase activity of PFKFB3/PFKFB4, however, new compounds and methods are needed. 3PO (3-(3-pyridinyl)-1-(4-pyridinyl)-2-propen-1-one) and ACT-PFK-158 ((E)-1-(pyridin-4-yl)-3-(7-(trifluoromethyl)quinolin-2-yl)prop-2-en-1-one) were reported to inhibit PFKFB3, reduce intracellular concentration of Fru-2,6-BP, reduce glucose uptake, and reduce growth of established tumors in vivo, however the PFKFB3 inhibition of 3PO is unclear based on a conflicting study. 3PO has been extensively used in research and was shown to inhibit proliferation of activated T-cells and inhibit angiogenesis.

New therapies which are able to regulate the role of PFKFB3 and PFKFB4 in cell metabolism and proliferation may prove useful in the treatment of a variety of pathologies.

### BRIEF DESCRIPTION OF THE DRAWINGS

FIG. 1 shows that GO-672 did not reduce cell viability of HUVECs both in normoxia and hypoxia conditions.

FIGS. 2A-2D show the effects of GO-672 on vessel sprouting in vitro.

FIG. 3 shows that PFKFB3 inhibitors protect neurons against MG132- and β-amyloid-induced toxicity.

FIG. 4A shows that AZ67 and Compound 1 at 0.01 μM restore NADPH/NADP ratio under excitotoxic conditions induced by glutamate.

FIG. 4B shows that AZ67 and Compound 1 at 0.01 μM significantly prevented the increase in mitochondrial ROS triggered both by NMDA and glutamate excitotoxic models in primary neurons.

FIG. 5 shows that both glutamate and NMDA significantly increased apoptotic neuronal death, an effect that was significantly prevented by AZ67 and by Compound 1.

FIG. 6A shows that AZ67 and Compound 1 protect mouse primary cortical neurons from apoptosis induced by NMDA and glutamate.

FIGS. 6B and 6C shows that PFK1-M overexpression was able to abolish the neuroprotection caused by AZ67 and Compound 1.

FIG. 7 shows an evaluation of the effect of PFKFB3 inhibitors on motor coordination after MCAO using a rota-rod test.

FIG. 8 shows an evaluation of the effect of PFKFB3 inhibitors on infarct volume of mice after MCAO.

FIG. 9 shows the effect of GO-672 as a single agent and in combination with cisplatin in a syngeneic model.

FIGS. 10A-10D show the effect of GO-672 in an orthotopic 4T1-M3-Luc syngeneic breast tumor model in female BALB/c mice in vivo in comparison with a vehicle/control group.

FIGS. 11A-11C show the effect of GO-672 in an orthotopic RENCA model in comparison with a vehicle/control group in female BALB/c mice.

FIG. 12 shows the rate of glycolysis in neurons of WT and Cln7 KO mice.

FIG. 13 shows the results of an valuation of apoptosis level in neurons by measuring active Caspase-3 using Western blot.

FIG. 14 shows the expression of PFKFB3 protein in Cln7 KO and WT mice measured by Western blot.

FIGS. 15A-15C show that AZ67 prevented the accumulation of SCMAS, lipofuscin, and reactive astroglia in the cortex of Cln7Aex2 mice in vivo.

FIG. 15D shows that hindlimb paralysis in Cln7Aex2 mice was prevented by AZ67, indicating functional recovery.

FIG. 16 shows that AZ67 rectified the loss of perinuclear mitochondria observed in induced pluripotent stems cells (iPSC)-derived neural precursor cells (NPC) from CLN7 patients.

FIGS. 17A and 17B show AzPfkb367 dose responses.

### BRIEF SUMMARY OF THE INVENTION

This disclosure relates to new phthalimide and isoindolinone derivatives as 6-phosphofructo-2-kinase/fructose-2,6-bisphosphatase-3 and -4 (PFKFB3, PFKFB4) and other PFKFB3 modulators and to pharmaceutical compositions comprising these compounds, and methods of using these compounds to reduce cellular glycolytic flux and/or treat and prevent cancer, inflammation, neurodegeneration, aging and other diseases and conditions, where the modulation of PFKFB3 and/or PFKFB4 has beneficial effect, in mammals, including humans and its use in manufacturing of the corresponding medicament and related kits.

In some embodiments, this disclosure relates to the new uses of agents deleting, reducing, binding, inhibiting or degrading PFKFB3, including but not limited to the known PFKFB3 inhibitors and their analogs and the inventions related to such new uses. PFKFB3 inhibitors can be useful for treatment of neurodegeneration, aging and aging-related diseases, disorders and conditions, can be used for rejuvenation and use in manufacturing of the corresponding medicament. The novel features of the invention are set forth with particularity in the appended claims and description. A better understanding of the features and advantages of the present invention will be obtained by reference to the following detailed description that sets forth illustrative embodiments, in which the principles of the invention are utilized, and the accompanying drawings

Disclosed herein are methods, agents, pharmaceutical compositions, and systems for anti-aging treatment and treatment of neurodegeneration, as well as relative systems, methods and kits.

In some embodiments, the removal or inhibition or degradation of PFKFB3 or modulation of Indirect Target as defined below is effective in decreasing the biological age of a patient or as other anti-aging treatment.

In some embodiments, the removal or inhibition or degradation of PFKFB3 or modulation of Indirect Target as defined below is effective in neuroprotection or treatment of neurodegenerative disease.

Disclosed herein is a compound of Formula (0):

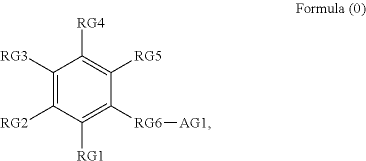

or a pharmaceutically acceptable salt thereof, wherein RG6 and RG5 is one of the following: A) RG6 and RG5 are taken together with the N to which they are attached to form a C₂-C₈ heterocycloalkyl optionally substituted with one or more substituents;

RG1, RG3 and RG4 are independently selected from R_{M}; RG2 is R_{L}; RG5 is Z; RG6 is —C(═O)—; AG1 is —Ar_{C}—Ar_{T};

thus the Formula (0) can be represented as the Formula (I):

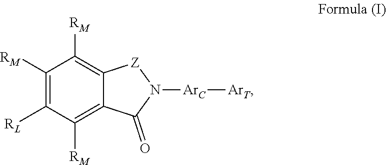

wherein: Z is selected from —C(═O)— and —C(R^{a})(R^{b})—; R^{a} and R^{b} are independently selected from hydrogen, hydroxyl, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₃-C₈ cycloalkyl, —O—C₃-C₈ cycloalkyl, C₂-C₈ heterocycloalkyl, and —O—C₂-C₈ heterocycloalkyl; and wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR¹R², —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

Ar_{c} is selected from C₃-C₈ cycloalkenylene, C₂-C₈ heterocycloalkenylene, arylene, and heteroarylene; wherein Ar_{c} is substituted with one or more R_{C};

each R_{C} are independently selected from —CN, —OH, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, optionally substituted —O—C₂-C₈ heterocycloalkyl, optionally substituted heteroaryl, optionally substituted aryl, —C(═O)OH, —C(═O)OR³, —C(═O)NR⁴R⁵, —S(═O)₂NR⁴R⁵, —NHC(═O)H, —NHC(═O)R⁶, —NHS(═O)₂R⁶, and —C(═O)NHS(═O)₂R⁶;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, —O—C₂-C₈ heterocycloalkyl, aryl, and heteroaryl are optionally substituted with one or more substituents independently selected from —OH, halogen, —C(═O)OR⁷, —C(═O)R⁶, —C(═O)NR¹R², C₁-C₆alkyl, C₁-C₆alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR⁷R⁸;

each R¹ and R² is independently selected from hydrogen, optionally substituted C₁-C₆ alkyl, and optionally substituted C₃-C₈ cycloalkyl;

wherein the C₁-C₆ alkyl is optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl is optionally substituted with one or more substituents independently selected from halogen, —C(═O)OR⁷, —C(═O)NR⁷R⁸, —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R³ is independently C₁-C₆ alkyl optionally substituted with one or more substituents independently selected from halogen, —OH, optionally substituted —OC(═O)C₁-C₆alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or each R³ is independently C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁴ and R⁵ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R⁶ are independently selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁷ and R⁸ is independently selected from hydrogen and C₁-C₆ alkyl;

or R⁷ and R⁸ are taken together with the N to which they are attached to form a C₂-C₈ heterocycloalkyl optionally substituted with one or more C₁-C₆alkyl substituents;

Ar_{T} is selected from pyridinyl, pyrimidinyl, pyridazinyl, pyrazinyl, phenyl, pyrrolyl, furanyl, thiophenyl, pyrazolyl, imidazolyl, triazolyl, and tetrazolyl, wherein Ar_{T} is optionally substituted by one or more substituents independently selected from halogen, —OH, —NR⁷R⁸, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;

each R_{M} is independently selected from hydrogen, halogen, —OH, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from —OH, —CN, optionally substituted C₁-C₆ hydroxyalkyl, optionally substituted C₁-C₆ alkoxy, optionally substituted heteroaryl, —C(═O)OH, —C(═O)OR⁹, —C(═O)NR¹⁰R¹¹, —S(═O)₂NR¹⁰R¹¹, —NHC(═O)H, —NHC(═O)R¹², —NHS(═O)₂R¹² and —C(═O)NHS(═O)₂R¹²;

wherein the C₁-C₆ hydroxyalkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the heteroaryl is optionally substituted with one or more of —OH, —O—C(═O)C₁-C₆ alkyl, (C₁-C₄ alkylene)-O—C(═O)C₁-C₆ alkyl, C₁-C₆ alkyl-(aryl), C₁-C₆ alkyl-(heteroaryl), halogen, —C(═O)OR⁷, —C(═O)R¹², —C(═O)NR¹R², C₁-C₆ alkyl, C₁-C₆ hydroxyalkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR¹R²;

R⁹ is C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁹ is C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R¹⁰ and R¹¹ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl (optionally substituted with —OH, halogen, —C(═O)OR⁷, —C(═O)NR⁷R⁸, C₁-C₆ alkyl, C₁-C₆ alkoxy, —NR⁷R⁸), and heteroaryl (optionally substituted with —OH, halogen, —C(═O)OR⁷, —C(═O)NR⁷R⁸, C₁-C₆ alkyl, C₁-C₆ alkoxy, —NR⁷R⁸, C₃-C₈ cycloalkyl, —O—C₃-C₈ cycloalkyl, C₂-C₈ heterocycloalkyl, or —O—C₂-C₈ heterocycloalkyl); and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R¹⁰ and R¹¹ are taken together with the N to which they are attached to form a C₂-C₈ heterocycloalkyl optionally substituted with one or more substituents independently selected from halogen, —OH, C₁-C₆ alkyl, and C₁-C₆ alkoxy;

R¹² is selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

provided that:

(a) at least one of R_{C} is not —NHCOR⁶ when R_{L} is —NHCOR¹² and Ar_{C} is heterocycloalkenylene or heteroarylene; or

(b) at least one of R_{C} is not -Me when R_{L} is —OMe; or

(c) at least one of R_{C} is not —OEt when R_{L} is —C(═O)OH; or

(d) at least one of R_{C} is not —OH when R_{L} is —C(═O)OH; or

(e) at least one of R_{C} is not -Me when R_{L} is —C(═O)OH; or

(f) at least one of R_{C} is not -Et when R_{L} is —OMe; or

(g) at least one of R_{C} is not optionally substituted benzoxazolyl when R_{L} is —C(═O)OH; or

(h) at least one of R_{C} is not optionally substituted isoindoline-1,3-dione when R_{L} is —C(═O)OH.

B) RG6 and RG5 do not form a C₂-C₈ heterocycloalkyl;

RG1 is R5; RG2 is R1; RG3 is R6; RG4 is R20; RG5 is R4; RG6 is R10; AG1 is A;

thus the Formula (0) can be represented as the Formula (VII):

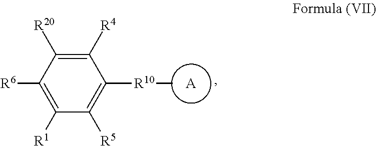

or a pharmaceutically acceptable salt thereof, wherein:

A is selected from:

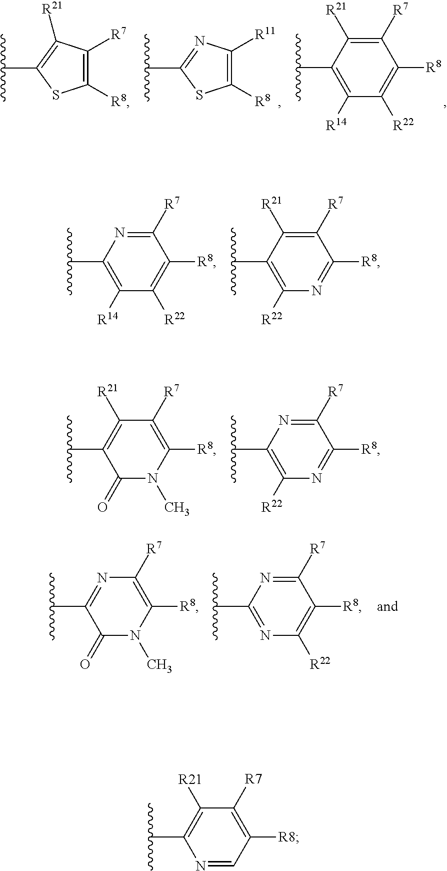

R¹ is selected from hydrogen, halogen, hydroxyl, C₁-C₆ alkyl, and C₁-C₆ alkoxy,

wherein the C₁-C₆ alkyl and C₁-C₆ alkoxy are optionally substituted with one or more halogens;

each R² and R³ is independently selected from hydrogen and C₁-C₆ alkyl,

wherein the C₁-C₆ alkyl is optionally substituted with one or more halogens;

or R² and R³ are taken together with the N to which they are attached to form a 3- to 10-membered heterocycle optionally substituted with one or more substituents independently selected from C₁-C₆ alkyl;

R⁴ is selected from hydrogen, halogen, C₁-C₆ alkyl, and C₁-C₆alkoxy,

wherein the C₁-C₆ alkyl and C₁-C₆ alkoxy are optionally substituted with one or more substituents independently selected from halogens;

R⁵ is selected from —C(═O)OR¹⁵, —C(═O)NR²R³, —S(═O)₂NR²R³, —C(═O)NHR¹⁵, —CH₂OH, 3-hydroxyoxetan-3-yl, and —NH₂;

R⁶ is selected from hydrogen, halogen, hydroxyl, 5-membered heteroaryl, C₁-C₆ alkyl, —C(═O)OR¹⁵, —C(═O)R¹², —C(═O)NHR¹⁵, and —C(═O)N═S(═X³)(CH₃)₂,

wherein the C₁-C₆ alkyl are optionally substituted with one or more R⁹, and

wherein 5-membered heteroaryl contains at least two heteroatoms and is optionally substituted with one or more substituents independently selected from R¹⁷;

R⁷ is selected from hydrogen, —NO₂, C₁-C₆ alkyl, C₁-C₆alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,

wherein the C₁-C₆ alkyl and C₁-C₆ alkoxy are optionally substituted with one or more halogens, and

wherein C₃-C₈ cycloalkyl, —O—C₃-C₈ cycloalkyl, 3- to 10-membered heterocycloalkyl, —O—(3- to 10-membered heterocycloalkyl), aryl and heteroaryl are optionally substituted with one or more R²⁴;

R⁸ is selected from hydrogen, —NO₂, C₁-C₆ alkyl, aryl, and heteroaryl,

wherein the C₁-C₆ alkyl and C₁-C₆ alkoxy are optionally substituted with one or more substituents independently selected at each occurrence from halogen; and

wherein aryl and heteroaryl are optionally substituted with one or more substituents independently selected at each occurrence from R²³;

or R⁷ and R⁸ are taken together to form a C₅-C₁₀ carbocycle or 5- to 10-membered heterocycle,

wherein C₅-C₁₀ carbocycle and 5- to 10-membered heterocycle are optionally substituted with one or more substituents independently selected from halogen, hydroxyl, —NO₂, C₁-C₆ alkyl, C₁-C₆ alkoxy, C₃-C₈ cycloalkyl, —O—C₃-C₈cycloalkyl, 3- to 10-membered heterocycloalkyl, —O—(3- to 10-membered heterocycloalkyl), aryl, and heteroaryl,

wherein the C₁-C₆ alkyl and C₁-C₆ alkoxy are optionally substituted with one or more halogens, and

wherein aryl, heteroaryl, C₃-C₈ cycloalkyl, —O—C₃-C₈ cycloalkyl, 3- to 10-membered heterocycloalkyl, and —O-(3- to 10-membered heterocycloalkyl) are optionally substituted with one or more R²³;

each R⁹ is independently selected from hydroxy and —COOH;

R¹⁰ is selected from —C(═O)—X¹—, —CH₂—X¹—, —X¹—C(═O)—, and —X¹—CH₂—;

R¹¹ is selected from hydrogen, —NO₂, C₁-C₆ alkyl, C₁-C₆alkoxy, C₃-C₈ cycloalkyl, —O—C₃-C₈ cycloalkyl, 3- to 10-membered heterocycloalkyl, and —O—(3- to 10-membered heterocycloalkyl),

wherein the C₁-C₆ alkyl and C₁-C₆ alkoxy are optionally substituted with one or more halogens, and

wherein C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;

R¹² is selected from alanine, arginine, asparagine, aspartic acid, asparagine, aspartic acid, cysteine, glutamic acid, glutamine, glutamine, glutamic acid, glycine, histidine, isoleucine, leucine, lysine, methionine, phenylalanine, proline, serine, threonine, tryptophan, tyrosine, and valine, wherein the point of attachment of R¹² is a nitrogen atom;

R¹⁴ is selected from hydrogen, halogen, hydroxyl, nitrile, —C(═O)CR¹⁵ and —C(═O)OR¹⁵;

each R¹⁵ is independently selected from hydrogen and C₁-C₆ alkyl, -heterocyclyl,

wherein the C₁-C₆ alkyl is optionally substituted with one or more substituents independently selected at each occurrence from —C(═O)NR²R³, -heterocyclyl, —NR²R³;

wherein the heterocyclyl is optionally substituted with one or more substituents independently selected at each occurrence from R² and R³.

R¹⁷ is selected from C₁-C₆ alkyl, aryl, and 6-membered heteroaryl,

wherein the C₁-C₆ alkyl is optionally substituted with one or more hydroxy, and

wherein aryl and 6-membered heteroaryl are optionally substituted with one or more substituents independently selected from halogen, —R², and —OR²;

R²⁰ is selected from hydrogen, halogen, hydroxyl, —COOH, —NC(═O)R², —OR², 5-membered heteroaryl, C₁-C₆ alkyl, —C(═O)N═S(═X³)(CH₃)₂, —CH₂(OH)CH₂OH and —NH—SO₂—R²,

wherein the 5-membered heteroaryl contains at least two heteroatoms, and

wherein the C₁-C₆ alkyl is optionally substituted with one or more substituents independently selected from 5-membered heteroaryl, wherein the 5-membered heteroaryl contains at least two heteroatoms;

R²¹ is selected from hydrogen and nitrile;

R²² is selected from hydrogen and hydroxy;

each R²³ is independently selected from halogen, C₁-C₆ alkyl, C₁-C₆ alkoxy,

wherein the C₁-C₆ alkyl and C₁-C₆ alkoxy are optionally substituted with one or more substituents independently selected from halogens;

each R²⁴ is independently selected from halogen, C₁-C₆ alkyl, C₁-C₆ alkoxy, 5-membered heteroaryl

wherein the C₁-C₆ alkyl and C₁-C₆ alkoxy are optionally substituted with one or more substituents independently selected from halogens;

each X¹ is independently selected from —NR²— and —CR²R³—; and

each X³ is independently selected from NH and O.

Disclosed herein also a compound of Formula (I):

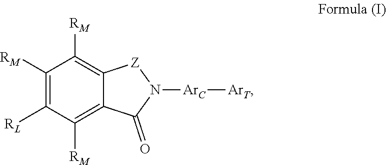

or a pharmaceutically acceptable salt thereof, wherein:

Z is selected from —C(═O)— and —C(R^{a})(R^{b})—;

R^{a} and R^{b} are independently selected from hydrogen, hydroxyl, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR¹R², —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

Ar_{c} is selected from C₃-C₈ cycloalkenylene, C₂-C₈ heterocycloalkenylene, arylene, and heteroarylene;

wherein Ar_{c} is substituted with one or more R_{C};

each R_{C} are independently selected from —CN, —OH, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, optionally substituted —O—C₂-C₈ heterocycloalkyl, optionally substituted heteroaryl, optionally substituted aryl, —C(═O)OH, —C(═O)OR³, —C(═O)NR⁴R⁵, —S(═O)₂NR⁴R⁵, —NHC(═O)H, —NHC(═O)R⁶, —NHS(═O)₂R⁶, and —C(═O)NHS(═O)₂R⁶;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, —O—C₂-C₈ heterocycloalkyl, aryl, and heteroaryl are optionally substituted with one or more substituents independently selected from —OH, halogen, —C(═O)OR⁷, —C(═O)R⁶, —C(═O)NR¹R², C₁-C₆alkyl, C₁-C₆ alkoxy, C₃-C₈ cycloalkyl, —O—C₃-C₈cycloalkyl, C₂-C₈ heterocycloalkyl, —O—C₂-C₈ heterocycloalkyl, optionally substituted aryl, optionally substituted heteroaryl, and —NR⁷R⁸;

each R¹ and R² is independently selected from hydrogen, optionally substituted C₁-C₆ alkyl, and optionally substituted C₃-C₈ cycloalkyl;

wherein the C₁-C₆ alkyl is optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl is optionally substituted with one or more substituents independently selected from halogen, —C(═O)OR⁷, —C(═O)NR⁷R⁸, —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R³ is independently C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or each R³ is independently C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁴ and R⁵ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R⁶ are independently selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁷ and R⁸ is independently selected from hydrogen and C₁-C₆ alkyl;

or R⁷ and R⁸ are taken together with the N to which they are attached to form a C₂-C₈ heterocycloalkyl optionally substituted with one or more C₁-C₆alkyl substituents;

Ar_{T} is selected from pyridinyl, pyrimidinyl, pyridazinyl, pyrazinyl, phenyl, pyrrolyl, furanyl, thiophenyl, pyrazolyl, imidazolyl, triazolyl, and tetrazolyl, wherein Ar_{T} is optionally substituted by one or more substituents independently selected from halogen, —OH, —NR⁷R⁸, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;

each R_{M} is independently selected from hydrogen, halogen, —OH, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl; R_{L} is selected from —OH, —CN, optionally substituted C₁-C₆ hydroxyalkyl, optionally substituted C₁-C₆ alkoxy, optionally substituted heteroaryl, —C(═O)OH, —C(═O)OR⁹, —C(═O)NR¹⁰R¹¹, —S(═O)₂NR¹⁰R¹¹, —NHC(═O)H, —NHC(═O)R¹², —NHS(═O)₂R¹² and —C(═O)NHS(═O)₂R¹²;

wherein the C₁-C₆ hydroxyalkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the heteroaryl is optionally substituted with one or more of —OH, —O—C(═O)C₁-C₆ alkyl, (C₁-C₄ alkylene)-O—C(═O)C₁-C₆ alkyl, C₁-C₆ alkyl-(aryl), C₁-C₆ alkyl-(heteroaryl), halogen, —C(═O)OR⁷, —C(═O)R¹², —C(═O)NR¹R², C₁-C₆ alkyl, C₁-C₆ hydroxyalkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR¹R²;

R⁹ is C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁹ is C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R¹⁰ and R¹¹ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl (optionally substituted with —OH, halogen, —C(═O)OR⁷, —C(═O)NR⁷R⁸, C₁-C₆ alkyl, C₁-C₆ alkoxy, —NR⁷R⁸), and heteroaryl (optionally substituted with —OH, halogen, —C(═O)OR⁷, —C(═O)NR⁷R⁸, C₁-C₆ alkyl, C₁-C₆ alkoxy, —NR⁷R⁸, C₃-C₈ cycloalkyl, —O—C₃-C₈ cycloalkyl, C₂-C₈ heterocycloalkyl, or —O—C₂-C₈ heterocycloalkyl); and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R¹⁰ and R¹¹ are taken together with the N to which they are attached to form a C₂-C₈ heterocycloalkyl optionally substituted with one or more substituents independently selected from halogen, —OH, C₁-C₆ alkyl, and C₁-C₆ alkoxy;

R¹² is selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

provided that:

(a) at least one of R_{C} is not —NHCOR⁶ when R_{L} is —NHCOR¹² and Ar_{C} is heterocycloalkenylene or heteroarylene; or

(b) at least one of R_{C} is not -Me when R_{L} is —OMe; or

(c) at least one of R_{C} is not —OEt when R_{L} is —C(═O)OH; or

(d) at least one of R_{C} is not —OH when R_{L} is —C(═O)OH; or

(e) at least one of R_{C} is not -Me when R_{L} is —C(═O)OH; or

(f) at least one of R_{C} is not -Et when R_{L} is —OMe; or

(g) at least one of R_{C} is not optionally substituted benzoxazolyl when R_{L} is —C(═O)OH; or

(h) at least one of R_{C} is not optionally substituted isoindoline-1,3-dione when R_{L} is —C(═O)OH.

In some embodiments of a compound of Formula (I), Z is —C(═O)—. In some embodiments of a compound of Formula (I), Z is —C(R^{a})(R^{b})—, and R^{a} and R^{b} are each independently selected from hydrogen, halogen, —OH, C₁₋₆ alkyl, and C₁₋₆ alkoxy. In some embodiments of a compound of Formula (I), Z is —C(R^{a})(R^{b})—, and R^{a} and R^{b} are each independently selected from hydrogen, fluorine, and methyl. In some embodiments of a compound of Formula (I), Z is —CH₂—.

In some embodiments of a compound of Formula (I), Ar_{C} is arylene or heteroarylene; each substituted with one or more R_{C}. In some embodiments of a compound of Formula (I), Ar_{C} is arylene substituted with one or two R_{C}. In some embodiments of a compound of Formula (I), Ar_{C} is a phenylene substituted with one or two R_{C}. In some embodiments of a compound of Formula (I), Ar_{C} is arylene substituted with one R_{C}. In some embodiments of a compound of Formula (I), Ar_{C} is phenylene substituted with one R_{C}. In some embodiments of a compound of Formula (I), Ar_{C} is heteroarylene substituted with one or two R_{C}. In some embodiments of a compound of Formula (I), Ar_{C} is a monocyclic heteroarylene substituted with one or two R_{C}. In some embodiments of a compound of Formula (I), Ar_{C} is heteroarylene substituted with one R_{C}. In some embodiments of a compound of Formula (I), Ar_{C} is thiophenylene substituted with one R_{C}. In some embodiments of a compound of Formula (I), Ar_{C} is thiophenylene substituted with two R_{C}.

In some embodiments of a compound of Formula (I), each R_{C} are independently selected from —CN, —OH, halogen, optionally substituted C₁-C₆ alkyl, optionally substituted C₃-C₈ cycloalkyl, optionally substituted C₁-C₆ alkoxy, optionally substituted heteroaryl, optionally substituted aryl, —C(═O)OH, —C(═O)OR³, and —C(═O)NR⁴R⁵; wherein the C₁-C₆ alkyl and C₁-C₆ alkoxy are optionally substituted with one or more substituent independently selected from halogen, —C(═O)OR⁷, —C(═O)NR¹R², —OH, aryl, heteroaryl, C₁-C₆ alkoxy, C₃-C₈ cycloalkyl, and C₂-C₈ heterocycloalkyl; and wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, and —NR⁷R⁸. In some embodiments of a compound of Formula (I), each R_{C} are independently selected from —CN, —OH, halogen, C₁-C₆ alkyl, C₁-C₆ hydroxyalkyl, C₁-C₆ hydroxycycloalkyl, C₁-C₆ alkoxy, heteroaryl, aryl, —C(═O)OH, —C(═O)OR³, and —C(═O)NR⁴R⁵. In some embodiments of a compound of Formula (I), one R_{C} is selected from —CN, —OH, halogen, C₁-C₆ alkyl, C₁-C₆ hydroxyalkyl, C₁-C₆ hydroxycycloalkyl, C₁-C₆ alkoxy, heteroaryl, aryl, —C(═O)OH, —C(═O)OR³, and —C(═O)NR⁴R⁵; and a second R_{C} is selected from —OH, halogen, C₁-C₆ alkyl, C₁-C₆ alkoxy, or aryl. In some embodiments of a compound of Formula (I), each R_{C} are independently selected from —CN, —C(═O)OH, —C(═O)OR³, and tetrazolyl. In some embodiments of a compound of Formula (I), one R_{C} is selected from —CN, —C(═O)OH, —C(═O)OR³, and tetrazolyl; and a second R_{C} is selected from —OH, halogen, C₁-C₆ alkyl, C₁-C₆ alkoxy, or aryl.

In some embodiments of a compound of Formula (I), each R³ is independently selected from C₁-C₆ alkyl optionally substituted with one or more of —OH, optionally substituted —OC(═O)C₁-C₆ alkyl, C₁-C₆ alkoxy, —C(═O)OH, and —NR¹R²; wherein the —OC(═O)C₁-C₆ alkyl is optionally substituted with one or more of —OH and —NR⁷R⁸. In some embodiments of a compound of Formula (I), each R³ is independently selected from C₁-C₆alkyl (optionally substituted with one or more of —OH, C₁-C₆alkoxy, and —NR¹R²) or —C₁-C₆alkylene-OC(═O)C₁-C₆alkyl (wherein C₁-C₆alkyl is optionally substituted with one or more of —OH and —NR⁷R⁸). In some embodiments of a compound of Formula (I), each R³ is independently C₁-C₆ alkyl optionally substituted with one or more of —OH, C₁-C₆ alkoxy, and —NR¹R². In some embodiments of a compound of Formula (I), each R³ is independently selected from

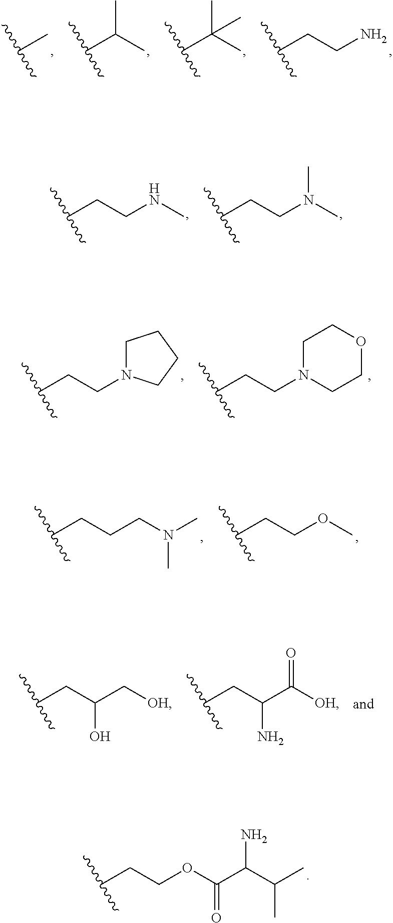

In some embodiments of a compound of Formula (I), each R⁴ and R⁵ is independently selected from hydrogen and C₁-C₆alkyl; or R⁴ and R⁵ are taken together with the N to which they are attached to form a C₂-C₈ heterocycloalkyl, optionally substituted with one or more C₁-C₆alkyl substituents. In some embodiments of a compound of Formula (I), each R⁴ and R⁵ are hydrogen.

In some embodiments of a compound of Formula (I), at least one of R_{C} is —CN. In some embodiments of a compound of Formula (I), at least one of R_{C} is —C(═O)OH. In some embodiments of a compound of Formula (I), at least one of R_{C} is tetrazolyl.

In some embodiments of a compound of Formula (I), Ar_{T} is selected from pyridinyl, pyrimidinyl, pyrazinyl, phenyl, thiophenyl, pyrazolyl, and imidazolyl, wherein Ar_{T} is optionally substituted by one or more substituents independently selected from halogen, —OH, —NR⁷R⁸, —CN, C₁-C₆ alkyl, and C₁-C₆ alkoxy. In some embodiments of a compound of Formula (I), Ar_{T} is phenyl optionally substituted by one or more substituents selected from halogen, —OH, —NR⁷R⁸, —CN, C₁-C₆alkyl, and C₁-C₆alkoxy.

In some embodiments of a compound of Formula (I), each R_{M} is independently selected from hydrogen, halogen, —OH, —CN, C₁-C₆alkyl, and C₁-C₆alkoxy. In some embodiments of a compound of Formula (I), one R_{M} is selected from hydrogen, halogen, —OH, —CN, C₁-C₆alkyl, and C₁-C₆alkoxy; and each other R_{M} is independently selected from hydrogen and halogen. In some embodiments of a compound of Formula (I), each R_{M} is hydrogen.

In some embodiments of a compound of Formula (I), R_{L} is selected from optionally substituted heteroaryl, —C(═O)OH, —C(═O)OR⁹, —C(═O)NR¹⁰R¹¹, —NHC(═O)R¹², —NHS(═O)₂R¹², and —C(═O)NHS(═O)₂R¹²;

wherein the heteroaryl is optionally substituted with one or more substituents independently selected from —OH, C₁-C₆ alkyl, C₁-C₆ hydroxyalkyl, C₁-C₆ alkoxy, —OC(═O)C₁-C₆ alkyl, (C₁-C₄ alkylene)-O—C(═O)C₁-C₆ alkyl, —C(═O)R¹², aryl, heteroaryl, C₁-C₆ alkyl-(aryl), and C₁-C₆ alkyl-(heteroaryl). In some embodiments of a compound of Formula (I), R_{L} is —C(═O)OR⁹. In some embodiments of a compound of Formula (I), R_{L} is —C(═O)OR⁹, R⁹ is C₁-C₆ alkylene-OC(═O)C₁-C₆ alkyl, wherein C₁-C₆ alkyl is optionally substituted with one or more of —OH and —NR⁷R⁸.

In some embodiments of a compound of Formula (I), R_{L} is —C(═O)OR⁹ and R⁹ is C₁-C₆ alkyl optionally substituted with —NR¹R². In some embodiments of a compound of Formula (I), R_{L} is —C(═O)OR⁹ and R⁹ is C₁-C₆ alkyl optionally substituted with —NR¹R² and each R¹ and R² is independently selected from hydrogen or C₁-C₆ alkyl. In some embodiments of a compound of Formula (I), R_{L} is —C(═O)OR⁹ and R⁹ is selected from

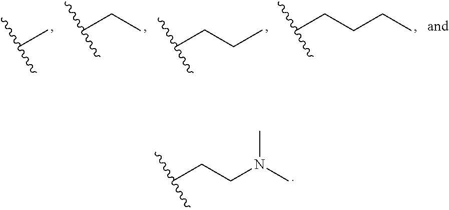

In some embodiments of a compound of Formula (I), R_{L} is —C(═O)NR¹⁰R¹¹, and each R¹⁰ and R¹¹ is independently selected from hydrogen and C₁-C₆ alkyl optionally substituted with one or more substituents independently selected from —C(═O)OH, —C(═O)NR¹R², —OH, aryl, and heteroaryl; or R¹⁰ and R¹¹ are taken together with the N to which they are attached to form a C₂-C₈ heterocycloalkyl, optionally substituted with one or more C₁-C₆ alkyl substituents. In some embodiments of a compound of Formula (I), R_{L} is —C(═O)NR¹⁰R¹¹; R¹⁰ is hydrogen; and R¹¹ is selected from hydrogen

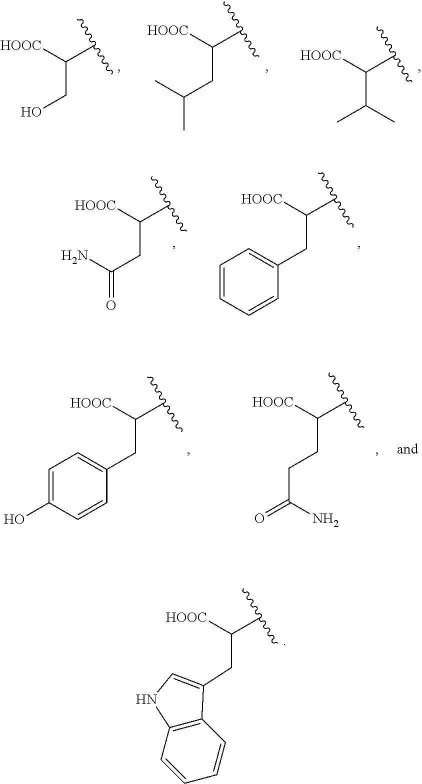

In some embodiments of a compound of Formula (I), R_{L} is selected from —NHC(═O)R¹², —NHS(═O)₂R¹², and —C(═O)NHS(═O)₂R¹², and R¹² is selected from C₁-C₆ alkyl and aryl optionally substituted with one or more C₁-C₆ alkyl substituents. In some embodiments of a compound of Formula (I), R_{L} is —NHC(═O)R¹²; and R¹² is methyl. In some embodiments of a compound of Formula (I), R_{L} is —NHS(═O)₂R¹²; and R¹² is selected from phenyl, toluyl, and methyl. In some embodiments of a compound of Formula (I), R_{L} is —C(═O)NHS(═O)₂R¹²; and R¹² is selected from methyl, butyl, and phenyl. In some embodiments of a compound of Formula (I), R_{L} is —C(═O)OH.

In some embodiments of a compound of Formula (I), R_{L} is monocyclic heteroaryl, optionally substituted with one or more substituents independently selected from —OH, C₁-C₆ alkyl, C₁-C₆ hydroxyalkyl, C₁-C₆ alkoxy, —OC(═O)C₁-C₆ alkyl, (C₁-C₄ alkylene)-O—C(═O)C₁-C₆ alkyl, —C(═O)R¹², aryl, heteroaryl, C₁-C₆ alkyl-(aryl), and C₁-C₆ alkyl-(heteroaryl). In some embodiments of a compound of Formula (I), R_{L} is tetrazolyl. In some embodiments of a compound of Formula (I), R_{L} is triazolyl, optionally substituted with one or more substituents independently selected from —OH, C₁-C₆ alkyl, C₁-C₆ hydroxyalkyl, C₁-C₆ alkoxy, —OC(═O)C₁-C₆ alkyl, (C₁-C₄ alkylene)-O—C(═O)C₁-C₆ alkyl, —C(═O)R¹², aryl, heteroaryl, C₁-C₆ alkyl-(aryl), and C₁-C₆ alkyl-(heteroaryl). In some embodiments of a compound of Formula (I), R_{L} is triazolyl.

In some embodiments of a compound of Formula (I), each R¹ and R² is independently selected from hydrogen and C₁-C₆ alkyl; or R¹ and R² are taken together with the N to which they are attached to form a C₂-C₈ heterocycloalkyl, optionally substituted with one or more C₁-C₆alkyl substituents. In some embodiments of a compound of Formula (I), each R¹, R², R⁷ and R⁸ is independently selected from hydrogen and C₁-C₆ alkyl. In some embodiments of a compound of Formula (I), each R¹ and R⁸ is hydrogen and each R² and R⁷ is independently selected from hydrogen and C₁-C₆ alkyl.

In some embodiments of a compound of Formula (I), R¹, R², R⁷ and R⁸ are each hydrogen. In some embodiments of a compound of Formula (I), the compound or a pharmaceutically acceptable salt thereof is in the form of a prodrug. In some embodiments of a compound of Formula (I), the compound or a pharmaceutically acceptable salt thereof is in the form of a prodrug and the prodrug comprises an ester moiety. In some embodiments of a compound of Formula (I), the compound or a pharmaceutically acceptable salt thereof is in the form of a prodrug and the prodrug comprises an amide moiety.

In certain embodiments, the disclosure provides compounds of Formula (Ia) or Formula (Ib):

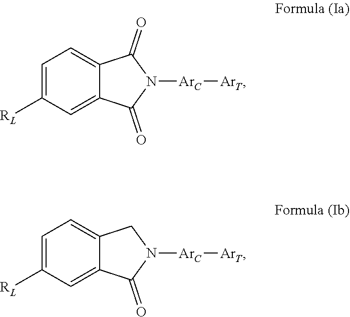

or a pharmaceutically acceptable salt thereof wherein:

Ar_{C} is selected from arylene and heteroarylene; wherein Ar_{C} is substituted with one or more R_{C};

each R_{C} are independently selected from halogen, —CN, optionally substituted C₁-C₆ alkyl, optionally substituted C₁-C₆ alkoxy, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

wherein the C₁-C₆ alkyl and C₁-C₆ alkoxy are optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl; and

wherein the aryl and heteroaryl are optionally substituted with one or more substituents independently selected from —OH, halogen, —C(═O)OH, —C(═O)NR¹R², C₁-C₆ alkyl, C₁-C₆ alkoxy, and —NR⁷R⁸;

each R¹ and R² is independently selected from hydrogen and C₁-C₆ alkyl optionally substituted with one or more substituents independently selected from halogen, —C(═O)OH, —C(═O)NR⁷R⁸, —OH, aryl, heteroaryl, C₃-C₈ cycloalkyl, and C₂-C₈ heterocycloalkyl;

or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R³ is independently C₁-C₆ alkyl optionally substituted with one or more substituents independently selected from —OH, optionally substituted —OC(═O)C₁-C₆ alkyl, optionally substituted —C(═O)OC₁-C₆ alkyl, C₁-C₆ alkoxy, —C(═O)OH, and —NR¹R²; wherein the —OC(═O)C₁-C₆ alkyl and —C(═O)OC₁-C₆ alkyl are optionally substituted with one or more substituents independently selected from —OH and —NR⁷R⁸;

each R⁴ and R⁵ is independently selected from hydrogen and C₁-C₆ alkyl optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl;

or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R⁶ are independently selected from optionally substituted C₁-C₆ alkyl and optionally substituted aryl;

wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and

wherein the aryl is optionally substituted with one or more substituents independently selected from —OH, halogen, —C(═O)OR⁷, —C(═O)NR⁷R⁸, C₁-C₆alkyl, C₁-C₆alkoxy, and —NR⁷R⁸;

each R⁷ and R⁸ is independently selected from hydrogen and C₁-C₆ alkyl;

or R⁷ and R⁸ are taken together with the N to which they are attached to form an optionally substituted C₂-C₈ heterocycloalkyl optionally substituted with one or more C₁-C₆ alkyl substituents;

Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²; wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹R², —C(═O)R¹², aryl, and C₁-C₆ alkyl-(aryl);

R⁹ is C₁-C₆ alkyl optionally substituted with one or more substituent independently selected from —OH and —NR¹R²;

each R¹⁰ and R¹¹ is independently selected from hydrogen and C₁-C₆ alkyl optionally substituted with one or more substituents independently selected from —C(═O)OH, —C(═O)NR¹R², —OH, aryl, hydroxyaryl and heteroaryl;

or R¹⁰ and R¹¹ are taken together with the N to which they are attached to form a C₂-C₈ heterocycloalkyl optionally substituted with one or more C₁-C₆ alkyl substituents;

R¹² is selected from C₁-C₆alkyl and aryl optionally substituted with one or more C₁-C₆alkyl substituents;

provided that at least one of R_{C} is not —OH when R_{L} is —C(═O)OH in Formula (Ia) or at least one of R_{C} is not —OEt when R_{L} is —C(═O)OH in Formula (Ia).

In some embodiments of a compound of Formula (Ia) or Formula (Ib), Ar_{C} is arylene substituted with one or two R_{C}. In some embodiments of a compound of Formula (Ia) or Formula (Ib), Ar_{C} is a monocyclic arylene substituted with one or two R_{C}. In some embodiments of a compound of Formula (Ia) or Formula (Ib), Ar_{C} is arylene substituted with one R_{C}. In some embodiments of a compound of Formula (Ia) or Formula (Ib), Ar_{C} is phenylene substituted with one R_{C}. In some embodiments of a compound of Formula (Ia) or Formula (Ib), Ar_{C} is heteroarylene substituted with one or two R_{C}. In some embodiments of a compound of Formula (Ia) or Formula (Ib), Ar_{C} is a monocyclic heteroarylene substituted with one or two R_{C}. In some embodiments of a compound of Formula (Ia) or Formula (Ib), Ar_{C} is heteroarylene substituted with one R_{C}. In some embodiments of a compound of Formula (Ia) or Formula (Ib), Ar_{C} is thiophenylene substituted with one R_{C}. In some embodiments of a compound of Formula (Ia) or Formula (Ib), Ar_{C} is thiophenylene substituted with two R_{C}.

In some embodiments of a compound of Formula (Ia) or Formula (Ib), each R_{C} are independently selected from —OH, —CN, halogen, C₁-C₆alkyl, C₁-C₆ alkoxy, C₁-C₆ hydroxyalkyl, heteroaryl, aryl, —C(═O)OH, —C(═O)OR³, and —C(═O)NR⁴R⁵. In some embodiments of a compound of Formula (Ia) or Formula (Ib), each R_{C} are independently selected from —CN, halogen, C₁-C₆alkyl, C₁-C₆ hydroxyalkyl, heteroaryl, aryl, —C(═O)OH, —C(═O)OR³, and —C(═O)NR⁴R⁵. In some embodiments of a compound of Formula (Ia) or Formula (Ib), one R_{C} is selected from —OH, —CN, C₁-C₆ hydroxyalkyl, heteroaryl, aryl, —C(═O)OH, —C(═O)OR³, and —C(═O)NR⁴R⁵; and a second R_{C} is selected from —OH, halogen, C₁-C₆ alkyl, C₁-C₆ alkoxy, and aryl. In some embodiments of a compound of Formula (Ia) or Formula (Ib), each R_{C} are independently selected from —CN, —C(═O)OH, —C(═O)OR³, and tetrazolyl. In some embodiments of a compound of Formula (Ia) or Formula (Ib), one R_{C} is selected from —CN, —C(═O)OH, —C(═O)OR³, and tetrazolyl; and a second R_{C} is selected from —OH, halogen, C₁-C₆ alkyl, C₁-C₆ alkoxy, and aryl.

In some embodiments of a compound of Formula (Ia) or Formula (Ib), each R³ is independently C₁-C₆ alkyl optionally substituted with one or more substituent selected from —OH, optionally substituted —OC(═O)C₁-C₆ alkyl, optionally substituted —C(═O)OC₁-C₆ alkyl, C₁-C₆ alkoxy, —C(═O)OH, —NR¹R²; wherein the —OC(═O)C₁-C₆ alkyl and —C(═O)OC₁-C₆ alkyl are optionally substituted with one or more substituent independently selected from —OH and —NR⁷R⁸. In some embodiments of a compound of Formula (Ia) or Formula (Ib), each R³ is independently C₁-C₆ alkyl optionally substituted with one or more substituents independently selected from C₁-C₆ alkoxy and —NR¹R². In some embodiments of a compound of Formula (Ia) or Formula (Ib), each R³ is independently selected from

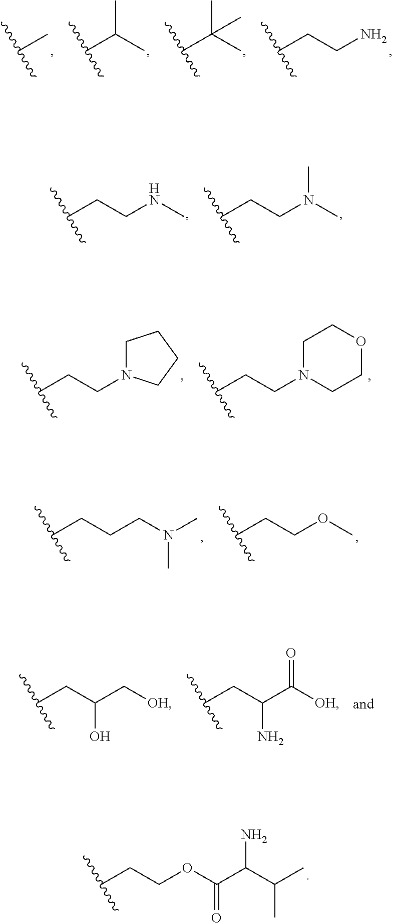

In some embodiments of a compound of Formula (Ia) or Formula (Ib), each R⁴ and R⁵ is independently selected from hydrogen and C₁-C₆ alkyl; or R⁴ and R⁵ are taken together with the N to which they are attached to form a C₂-C₈ heterocycloalkyl, optionally substituted with one or more C₁-C₆ alkyl substituents. In some embodiments of a compound of Formula (Ia) or Formula (Ib), each R⁴ and R⁵ is hydrogen. In some embodiments of a compound of Formula (Ia) or Formula (Ib), at least one of R_{C} is —CN. In some embodiments of a compound of Formula (Ia) or Formula (Ib), at least one of R_{C} is —C(═O)OH. In some embodiments of a compound of Formula (Ia) or Formula (Ib), at least one of R_{C} is tetrazolyl.

In some embodiments of a compound of Formula (Ia) or Formula (Ib), Ar_{T} is phenyl optionally substituted by one or more substituents independently selected from halogen, —OH, —NR⁷R⁸, —CN, C₁-C₆ alkyl, and C₁-C₆ alkoxy. In some embodiments of a compound of Formula (Ia) or Formula (Ib), Ar_{T} is selected from pyridinyl, pyrimidinyl, pyrazinyl, thiophenyl, pyrazolyl and imidazolyl, wherein Ar_{T} is optionally substituted by one or more substituents independently selected from halogen, —OH, —NR⁷R⁸, —CN, C₁-C₆ alkyl, and C₁-C₆ alkoxy. In some embodiments of a compound of Formula (Ia) or Formula (Ib), Ar_{T} is selected from thiophenyl, pyrazolyl, and imidazolyl, wherein Ar_{T} is optionally substituted by one or more substituents independently selected from halogen, —OH, —NR⁷R⁸, —CN, C₁-C₆alkyl, and C₁-C₆alkoxy. In some embodiments of a compound of Formula (Ia) or Formula (Ib), Ar_{T} is imidazolyl optionally substituted by methyl.

In some embodiments of a compound of Formula (Ia) or Formula (Ib), R_{L} is —C(═O)OR⁹. In some embodiments of a compound of Formula (Ia) or Formula (Ib), R_{L} is —C(═O)OR⁹ and R⁹ is selected from

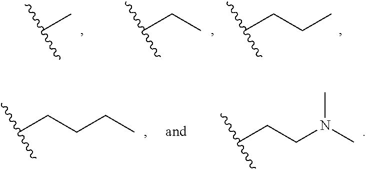

In some embodiments of a compound of Formula (Ia) or Formula (Ib), R_{L} is —C(═O)NR¹⁰R¹¹; R¹⁰ is hydrogen; and R¹¹ is selected from hydrogen,

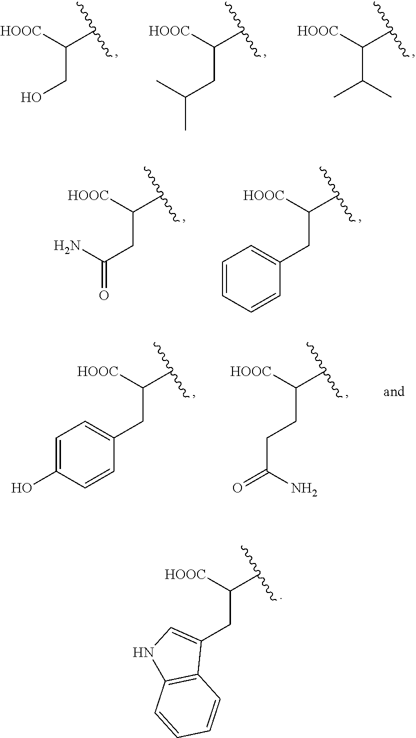

In some embodiments of a compound of Formula (Ia) or Formula (Ib), R_{L} is —NHC(═O)R¹² and R¹² is methyl. In some embodiments of a compound of Formula (Ia) or Formula (Ib), R_{L} is —NHS(═O)₂R¹² and R¹² is selected from phenyl, toluyl, and methyl. In some embodiments of a compound of Formula (Ia) or Formula (Ib), R_{L} is —C(═O)NHS(═O)R¹². In some embodiments of a compound of Formula (Ia) or Formula (Ib), R_{L} is —C(═O)NHS(═O)R¹² and R¹² is selected from methyl, butyl, and phenyl. In some embodiments of a compound of Formula (Ia) or Formula (Ib), R_{L} is —C(═O)OH. In some embodiments of a compound of Formula (Ia) or Formula (Ib), R_{L} is tetrazolyl. In some embodiments of a compound of Formula (Ia) or Formula (Ib), R_{L} is triazolyl, optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹R², —C(═O)R¹², aryl, and C₁-C₆ alkyl-(aryl). In some embodiments of a compound of Formula (Ia) or Formula (Ib), R_{L} is triazolyl.

In some embodiments of a compound of Formula (Ia) or Formula (Ib), each R¹ and R² is independently selected from hydrogen and C₁-C₆ alkyl, or R¹ and R² are taken together with the N to which they are attached to form an optionally substituted C₂-C₈ heterocycloalkyl optionally substituted with one or more C₁-C₆alkyl substituents. In some embodiments of a compound of Formula (Ia) or Formula (Ib), each R¹, R², R⁷ and R⁸ is hydrogen.

In some embodiments of a compound of Formula (Ia) or Formula (Ib), the compound or a pharmaceutically acceptable salt thereof is in the form of a prodrug. In some embodiments of a compound of Formula (Ia) or Formula (Ib), the compound or a pharmaceutically acceptable salt thereof is in the form of a prodrug and the prodrug comprises an ester moiety. In some embodiments of a compound of Formula (Ia) or Formula (Ib), the compound or a pharmaceutically acceptable salt thereof is in the form of a prodrug and the prodrug comprises an amide moiety.

In certain embodiments, the disclosure provides compounds of Formula (II):

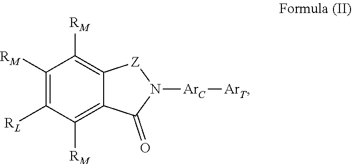

a prodrug thereof, a pharmaceutically acceptable salt thereof, or combination thereof, wherein:

Z is —C(═O)— or —C(R^{a})(R^{b})—;

R^{a} and R^{b} are each independently selected from hydrogen, halogen, —OH, C₁-C₆ alkyl, C₁-C₆ alkoxy;

Ar_{C} is selected from arylene and heteroarylene; wherein Ar_{C} is substituted with one or more R_{C};

each R_{C} is independently selected from halogen, —CN, optionally substituted C₁-C₆ alkyl, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, and C₂-C₈ heterocycloalkyl; and

wherein the aryl and heteroaryl are optionally substituted with one or more substituents independently selected from —OH, halogen, —C(═O)OR⁷, —C(═O)NR¹R², C₁-C₆ alkyl, C₁-C₆ alkoxy, and —NR⁷R⁸;

each R¹ and R² is independently selected from hydrogen and C₁-C₆ alkyl optionally substituted with one or more substituents independently selected from halogen, —C(═O)OR⁷, —C(═O)NR⁷R⁸, —OH, aryl, heteroaryl, C₃-C₈ cycloalkyl, and C₂-C₈ heterocycloalkyl;

or R¹ and R² are taken together with the N to which they are attached to form a C₂-C₈ heterocycloalkyl optionally substituted with one or more C₁-C₆ alkyl substituents;

each R³ is independently C₁-C₆ alkyl optionally substituted with one or more substituents independently selected from —OH, optionally substituted —OC(═O)C₁-C₆alkyl, optionally substituted —C(═O)OC₁-C₆ alkyl, C₁-C₆ alkoxy, —C(═O)OH, —NR¹R²;

wherein the —OC(═O)C₁-C₆ alkyl and —C(═O)OC₁-C₆ alkyl are optionally substituted with one or more substituents independently selected from with —OH or —NR⁷R⁸;

each R⁴ and R⁵ is independently selected from hydrogen and C₁-C₆ alkyl optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl;

or R⁴ and R⁵ are taken together with the N to which they are attached to form a C₂-C₈ heterocycloalkyl optionally substituted with one or more C₁-C₆ alkyl substituents;

each R⁶ are independently selected from optionally substituted C₁-C₆ alkyl and optionally substituted aryl;

wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and

wherein the aryl is optionally substituted with one or more substituents independently selected from —OH, halogen, —C(═O)OR⁷, —C(═O)NR⁷R⁸, C₁-C₆ alkyl, C₁-C₆ alkoxy, and —NR⁷R⁸;

each R⁷ and R⁸ is independently selected from hydrogen and C₁-C₆ alkyl;

or R⁷ and R⁸ are taken together with the N to which they are attached to form an optionally substituted C₂-C₈ heterocycloalkyl optionally substituted with one or more C₁-C₆ alkyl substituents;

Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;

each R_{M} is independently selected from hydrogen, halogen, —OH, —CN, optionally substituted C₁-C₆alkyl, and optionally substituted C₁-C₆ alkoxy;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;

wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹⁰R¹¹, —C(═O)R¹², aryl, or C₁-C₆ alkyl-(aryl);

R⁹ is C₁-C₆ alkyl optionally substituted with one or more substituents independently selected from —OH and —NR¹R²;

each R¹⁰ and R¹¹ is independently selected from hydrogen and C₁-C₆ alkyl optionally substituted with one or more substituents independently selected from —C(═O)OR⁷, —C(═O)NR¹R², —OH, aryl, hydroxyaryl or heteroaryl;

or R¹⁰ and R¹¹ are taken together with the N to which they are attached to form a C₂-C₈ heterocycloalkyl optionally substituted with one or more C₁-C₆ alkyl substituents;

R¹² is selected from C₁-C₆ alkyl and aryl optionally substituted with one or more C₁-C₆ alkyl substituents; and

wherein at least one R_{C} is —C(═O)OH; or R_{L} is —C(═O)OH.

In some embodiments of a compound of Formula (II), Z is —C(═O)—. In some embodiments of a compound of Formula (II), Z is —C(R^{a})(R^{b})—, and R^{a} and R^{b} are each independently selected from hydrogen, fluorine and methyl. In some embodiments of a compound of Formula (II), Z is —CH₂—. In some embodiments of a compound of Formula (II), each R_{M} is independently selected from hydrogen, halogen, —OH, —CN, C₁-C₆ alkyl, and C₁-C₆ alkoxy. In some embodiments of a compound of Formula (II), one R_{M} is selected from hydrogen, halogen, —OH, —CN, C₁-C₆alkyl, and C₁-C₆ alkoxy; and each other R_{M} is independently selected from hydrogen and halogen. In some embodiments of a compound of Formula (II), each R_{M} is hydrogen. In some embodiments of a compound of Formula (II), R_{L} is —C(═O)OH.

In certain embodiments, the disclosure provides compounds of Formula (III):

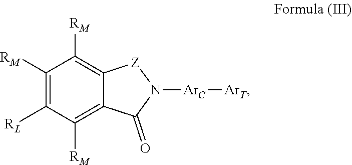

or a pharmaceutically acceptable salt thereof, wherein: Z is —C(═O)— or —C(R^{a})(R^{b})—;

R^{a} and R^{b} are each independently selected from hydrogen, halogen, —OH, C₁-C₆ alkyl, C₁-C₆ alkoxy;

Ar_{C} is selected from arylene and heteroarylene; wherein Ar_{C} is substituted with one or more R_{C};

each R_{C} is independently selected from halogen, —CN, optionally substituted C₁-C₆ alkyl, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, and C₂-C₈ heterocycloalkyl; and

wherein the aryl and heteroaryl are optionally substituted with one or more substituents independently selected from —OH, halogen, —C(═O)OR⁷, —C(═O)NR¹R², C₁-C₆ alkyl, C₁-C₆ alkoxy, and —NR⁷R⁸;

each R¹ and R² is independently selected from hydrogen and C₁-C₆ alkyl optionally substituted with one or more substituents independently selected from halogen, —C(═O)OR⁷, —C(═O)NR⁷R⁸, —OH, aryl, heteroaryl, C₃-C₈ cycloalkyl, and C₂-C₈ heterocycloalkyl;

or R¹ and R² are taken together with the N to which they are attached to form a C₂-C₈ heterocycloalkyl optionally substituted with one or more C₁-C₆ alkyl substituents;

each R³ is independently C₁-C₆ alkyl optionally substituted with one or more substituents independently selected from —OH, optionally substituted —OC(═O)C₁-C₆alkyl, optionally substituted —C(═O)OC₁-C₆ alkyl, C₁-C₆ alkoxy, —C(═O)OH, —NR¹R²;

wherein the —OC(═O)C₁-C₆ alkyl and —C(═O)OC₁-C₆ alkyl are optionally substituted with one or more substituents independently selected from with —OH or —NR⁷R⁸;

each R⁴ and R⁵ is independently selected from hydrogen and C₁-C₆ alkyl optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl;

or R⁴ and R⁵ are taken together with the N to which they are attached to form a C₂-C₈ heterocycloalkyl optionally substituted with one or more C₁-C₆ alkyl substituents;

R⁶ is selected from optionally substituted C₁-C₆ alkyl and optionally substituted aryl;

wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and

wherein the aryl is optionally substituted with one or more substituents independently selected from —OH, halogen, —C(═O)OR⁷, —C(═O)NR⁷R⁸, C₁-C₆ alkyl, C₁-C₆ alkoxy, and —NR⁷R⁸;

each R⁷ and R⁸ is independently selected from hydrogen and C₁-C₆ alkyl;

or R⁷ and R⁸ are taken together with the N to which they are attached to form an optionally substituted C₂-C₈ heterocycloalkyl optionally substituted with one or more C₁-C₆ alkyl substituents;

Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;

each R_{M} is independently selected from hydrogen, halogen, —OH, —CN, optionally substituted C₁-C₆ alkyl, and optionally substituted C₁-C₆ alkoxy;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;

wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹⁰R¹¹, —C(═O)R¹², aryl, or C₁-C₆ alkyl-(aryl);

R⁹ is C₁-C₆ alkyl optionally substituted with one or more substituents independently selected from —OH and —NR¹R²;

each R¹⁰ and R¹¹ is independently selected from hydrogen and C₁-C₆ alkyl optionally substituted with one or more substituents independently selected from —C(═O)OH, —C(═O)NR¹R², —OH, aryl, hydroxyaryl or heteroaryl;

or R¹⁰ and R¹¹ are taken together with the N to which they are attached to form a C₂-C₈ heterocycloalkyl optionally substituted with one or more C₁-C₆ alkyl substituents;

R¹² is selected from C₁-C₆ alkyl and aryl optionally substituted with one or more C₁-C₆ alkyl substituents; and

wherein at least one R_{C} is —C(═O)OR³ or R_{L} is —C(═O)OR⁹.

In some embodiments of a compound of Formula (III), Z is —C(═O)—. In some embodiments of a compound of Formula (III), Z is —C(R^{a})(R^{b})—, wherein R^{a} and R^{b} are each independently selected from hydrogen, fluorine and methyl. In some embodiments of a compound of Formula (III), Z is —CH₂—. In some embodiments of a compound of Formula (III), each R_{M} is independently selected from hydrogen, halogen, —OH, —CN, C₁-C₆ alkyl, and C₁-C₆ alkoxy. In some embodiments of a compound of Formula (III), one R_{M} is selected from hydrogen, halogen, —OH, —CN, C₁-C₆ alkyl, and C₁-C₆alkoxy; and each other R_{M} is independently selected from hydrogen and halogen. In some embodiments of a compound of Formula (III), each R_{M} is hydrogen.

In certain embodiments, the disclosure provides compounds of Formula (IV):

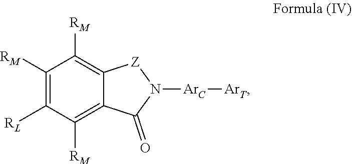

or a pharmaceutically acceptable salt thereof, wherein:

Z is —C(═O)— or —C(R^{a})(R^{b})—;

R^{a} and R^{b} are each independently selected from hydrogen, fluorine and methyl;

Ar_{C} is selected from arylene and heteroarylene; each substituted with one or more R_{C};

R_{C} is selected from —CN, —OH, C₁-C₆ alkoxy, C₁-C₆ alkyl, C₁-C₆ hydroxyalkyl, heteroaryl, aryl, —C(═O)OH, and —C(═O)OR³;

each R¹ and R² is independently selected from hydrogen and C₁-C₆ alkyl;

or R¹ and R² are taken together with the N to which they are attached to form a C₂-C₈ heterocycloalkyl;

each R³ is independently C₁-C₆alkyl optionally substituted with one or more —NR¹R² or C₁-C₆ alkoxy;

Ar_{T} is selected from pyridinyl, phenyl, thiophenyl, pyrazolyl, imidazolyl, and tetrazolyl, wherein Ar_{T} is optionally substituted by one or more substituents independently selected from halogen, —NR⁷R⁸, C₁-C₆ alkyl, and C₁-C₆ alkoxy;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one or more substituents independently selected from (C₁-C₄ alkylene)-O—C(═O)C₁-C₆ alkyl, —C(═O)NR¹R², —C(═O)R¹², aryl, and C₁-C₆ alkyl-(aryl).

each R¹⁰ and R¹¹ is independently selected from hydrogen and C₁-C₆ alkyl optionally substituted with one or more of —C(═O)OH, —OH, aryl, hydroxyaryl, or heteroaryl; and

R¹² is selected from C₁-C₆ alkyl and aryl.

In some embodiments of a compound of Formula (IV):

is —C(═O)— or —CH₂—;

Ar_{C} is selected from phenylene and monocyclic heteroarylene; each substituted with one or more R_{C};

R_{C} is selected from —CN, —OH, C₁-C₆ alkoxy, C₁-C₆ alkyl, heteroaryl, aryl, —C(═O)OH, —C(═O)OR³;

each R¹ and R² is independently selected from hydrogen and C₁-C₆ alkyl;

each R³ is independently C₁-C₆alkyl optionally substituted with one or more —NR¹R²;

Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;

each R_{M} is hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one or more substituents independently selected from —C(═O)R¹² and aryl.

each R¹⁰ and R¹¹ is independently selected from hydrogen and optionally substituted C₁-C₆alkyl; wherein the C₁-C₆ alkyl is optionally substituted by one or more substituents independently selected from —C(═O)OH, —OH, aryl, hydroxyaryl, and heteroaryl; and

R¹² is C₁-C₆ alkyl or aryl.

In some embodiments of a compound of Formula (IV):

Z is selected from —C(═O)— and —CH₂—;

Ar_{C} is arylene substituted with one R_{C};

R_{C} is selected from —C(═O)OH and tetrazolyl;

Ar_{T} is selected from pyridinyl, phenyl, thiophenyl, pyrazolyl, imidazolyl, and tetrazolyl, wherein Ar_{T} is optionally substituted by one or more of halogen, C₁-C₆ alkyl, and C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein heteroaryl is optionally substituted by one of —C(═O)R¹² or aryl;

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, phenyl, hydroxyphenyl, and indolyl; and

R¹² is C₁-C₆ alkyl.

In some embodiments of a compound of Formula (IV), Ar_{C} is phenylene. In some embodiments of a compound of Formula (IV), R_{L} is triazolyl optionally substituted by one of —C(═O)R¹² or aryl.

In some embodiments of a compound of Formula (IV):

Z is selected from —C(═O)— and —CH₂—;

Ar_{C} is heteroarylene substituted with one or two R_{C};

each R_{C} is independently selected from —CN, C₁-C₆ alkyl, and aryl;

Ar_{T} is phenyl optionally substituted by one or more substituents independently selected from halogen, C₁-C₆alkyl, or C₁-C₆alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and C₁-C₆ alkyl optionally substituted by one or more substituents independently selected from —C(═O)OH, —OH, phenyl, hydroxyphenyl, and indolyl; and

R¹² is C₁-C₆ alkyl.

In some embodiments of a compound of Formula (IV), Ar_{C} is thiophenylene. In some embodiments of a compound of Formula (IV), R_{L} is triazolyl optionally substituted by one of —C(═O)R¹² or aryl.

In some embodiments of a compound of Formula (IV), one of R_{C} is —CN.

In some embodiments of a compound of Formula (IV):

Z is selected from —C(═O)— and —CH₂—;

Ar_{C} is arylene substituted with one R_{C};

R_{C} is —C(═O)OR³;

R¹ and R² is independently selected from hydrogen and C₁-C₆ alkyl;

R³ is C₁-C₆ alkyl optionally substituted with one NR¹R²;

Ar_{T} is phenyl optionally substituted by one or more substituents independently selected from halogen, C₁-C₆ alkyl, and C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and C₁-C₆ alkyl optionally substituted by one or more substituents independently selected from —C(═O)OH, —OH, phenyl, hydroxyphenyl, and indolyl; and

R¹² is C₁-C₆ alkyl.

In some embodiments of a compound of Formula (IV), Ar_{C} is phenylene. In some embodiments of a compound of Formula (IV), R_{L} is triazolyl optionally substituted by one of —C(═O)R¹² or aryl.

In some embodiments of a compound of Formula (IV):

Z is —C(═O)—;

Ar_{C} is arylene substituted with one R_{C};

R_{C} is selected from —C(═O)OH and tetrazolyl;

Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and C₁-C₆ alkyl optionally substituted by one or more substituents independently selected from —C(═O)OH, —OH, aryl, hydroxyaryl and heteroaryl; and

R¹² is C₁-C₆ alkyl.

In some embodiments of a compound of Formula (IV), Ar_{C} is phenylene. In some embodiments of a compound of Formula (IV), R_{L} is triazolyl optionally substituted by one of —C(═O)R¹² or aryl.

In some embodiments of a compound of Formula (IV):

Z is —C(═O)—;

Ar_{C} is heteroarylene substituted with one or two R_{C};

each R_{C} is independently selected from —CN, C₁-C₆ alkyl, and aryl;

Ar_{T} is phenyl optionally substituted by one or more of halogen, C₁-C₆ alkyl, or C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, aryl, hydroxyaryl or heteroaryl; and

R¹² is C₁-C₆ alkyl.

In some embodiments of a compound of Formula (IV), Ar_{C} is thiophenylene. In some embodiments of a compound of Formula (IV), R_{L} is triazolyl optionally substituted by one of —C(═O)R¹² or aryl. In some embodiments of a compound of Formula (IV), one of R_{C} is —CN.

In some embodiments of a compound of Formula (IV):

Z is —C(═O)—;

Ar_{C} is arylene substituted with one R_{C};

R_{C} is —C(═O)OR³;

R¹ and R² is independently selected from hydrogen and C₁-C₆ alkyl;

R³ is C₁-C₆ alkyl optionally substituted with one —NR¹R²;

Ar_{T} is phenyl optionally substituted by one or more of halogen, C₁-C₆ alkyl, or C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

R¹⁰ is selected from hydrogen and C₁-C₆ alkyl;

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, aryl, hydroxyaryl, and heteroaryl; and

R¹² is C₁-C₆ alkyl.

In some embodiments of a compound of Formula (IV), Ar_{C} is phenylene. In some embodiments of a compound of Formula (IV), R_{L} is triazolyl optionally substituted by one of —C(═O)R¹² or aryl.

In certain embodiments, the disclosure provides compounds of Formula (V):

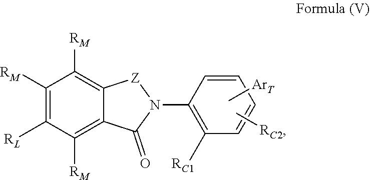

or a pharmaceutically acceptable salt thereof, wherein:

Z is —C(═O)— or —C(R^{a})(R^{b})—;

R^{a} and R^{b} are each independently selected from hydrogen, fluorine and methyl;

R_{C1} is selected from —OH, tetrazolyl, —C(═O)OH, and —C(═O)OR³;

R_{C2} is selected from hydrogen, halogen, —OH, C₁-C₆ alkyl, C₁-C₆ hydroxyalkyl and C₁-C₆ alkoxy;

R³ is C₁-C₆ alkyl optionally substituted with one or more substituent selected from —NR¹R² or C₁-C₆ alkoxy;

each R¹ and R² is independently selected from hydrogen and C₁-C₆ alkyl;

or R¹ and R² are taken together with the N to which they are attached to form a C₂-C₈ heterocycloalkyl;

Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

R¹⁰ is selected from hydrogen and C₁-C₆ alkyl;

R¹¹ is selected from hydrogen and optionally substituted C₁-C₆ alkyl; wherein the C₁-C₆ alkyl is optionally substituted with one or more of —C(═O)OH, —OH, aryl, hydroxyaryl, and heteroaryl; and

R¹² is selected from C₁-C₆ alkyl and aryl.

In some embodiments of a compound of Formula (V), R_{C1} is tetrazolyl or —C(═O)OH. In some embodiments of a compound of Formula (V), is —C(═O)OR³. In some embodiments of a compound of Formula (V), R_{C2} is hydrogen. In some embodiments of a compound of Formula (V), Ar_{T} is selected from pyridinyl, phenyl and thiophenyl, each optionally substituted by one or more substituents independently selected from halogen, C₁-C₆ alkyl, and C₁-C₆ alkoxy. In some embodiments of a compound of Formula (V), Ar_{T} is pyrazolyl or imidazolyl each optionally substituted by methyl. In some embodiments of a compound of Formula (V), R_{L} is triazolyl optionally substituted by one of —C(═O)R¹² or aryl.

Also disclosed herein are compounds of Formula (VI):

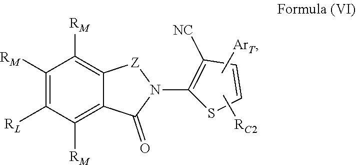

or a pharmaceutically acceptable salt thereof, wherein:

Z is —C(═O)— or —C(R^{a})(R^{b})—;

R^{a} and R^{b} are each independently selected from hydrogen, fluorine and methyl;

R_{C2} is selected from hydrogen, halogen, —OH, C₁-C₆ alkyl, C₁-C₆ hydroxyalkyl, C₁-C₆ alkoxy and aryl;

Ar_{T} is phenyl optionally substituted by one or more substituents independently selected from halogen, C₁-C₆ alkyl, and C₁-C₆ alkoxy;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

R¹⁰ is selected from hydrogen and C₁-C₆ alkyl;

R¹¹ is selected from hydrogen and optionally substituted C₁-C₆ alkyl; wherein the C₁-C₆ alkyl is optionally substituted with one or more of —C(═O)OH, —OH, aryl, hydroxyaryl, and heteroaryl; and

R¹² is selected from C₁-C₆ alkyl and aryl.

In some embodiments of a compound of Formula (VI), R_{C2} is selected from C₁-C₆ alkyl and phenyl. In some embodiments of a compound of Formula (VI), Z is —C(═O)—. In some embodiments of a compound of Formula (VI), Z is —CH₂—. In some embodiments of a compound of Formula (VI), each R_{M} is hydrogen. In some embodiments of a compound of Formula (VI), R_{L} is monocyclic heteroaryl optionally substituted by one of —C(═O)R¹² or aryl. In some embodiments of a compound of Formula (VI), R_{L} is tetrazolyl. In some embodiments of a compound of Formula (VI), R_{L} is triazolyl optionally substituted by one of —C(═O)R¹² or aryl. In some embodiments of a compound of Formula (VI), R_{L} is —C(═O)OH. In some embodiments of a compound of Formula (VI), R_{L} is —C(═O)NR¹⁰R¹¹, wherein R¹⁰ is selected from hydrogen and C₁-C₆ alkyl; and R¹¹ is selected from hydrogen and C₁-C₆ alkyl (optionally substituted with one or more of —C(═O)OH, —OH, phenyl, hydroxyphenyl, or indolyl). In some embodiments of a compound of Formula (VI), R_{L} is —C(═O)NHS(═O)₂R¹². In some embodiments of a compound of Formula (VI), the compound or a pharmaceutically acceptable salt thereof is in the form of a prodrug. In some embodiments of a compound of Formula (VI), the prodrug comprises an ester moiety. In some embodiments of a compound of Formula (VI), the prodrug comprises an amide moiety.

Also disclosed herein is a pharmaceutical composition comprising a compound of Formula (0), (0), (I), (Ia), (Ib), (II), (III), (IV), (V), (VI), (VII), (VIIA) or (VIIB) and one or more pharmaceutically acceptable carrier. Also disclosed herein is a pharmaceutical composition comprising a compound of Formula (0), (0), (I), (Ia), (Ib), (II), (III), (IV), (V), (VI), (VII), (VIIA) or (VIIB) in combination with another therapeutic agent, and optionally, one or more pharmaceutically acceptable carriers. In some embodiments, the pharmaceutical composition further comprises a second therapeutic agent. In some embodiments, the second therapeutic agent is an anti-cancer agent.

Also disclosed herein is a method of inhibition of the glycolysis in a cell, comprising contacting the cell with an effective amount of a compound of Formula (0), (I), (Ia), (Ib), (II), (III), (IV), (V), (VI), (VII), (VIIA) or (VIIB).

Also disclosed herein is a method of modulating the activity of PFKFB3 and/or PFKFB4 in a cell, comprising contacting the cell with an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of inhibition of PFKFB3 and/or PFKFB4 in a cell, comprising contacting the cell with an effective amount of a compound of Formula (0), (I), (Ia), (Ib), (II), (III), (IV), (V), (VI), (VII), (VIIA) or (VIIB).

Also disclosed herein is a method of inhibiting 6-phosphofructo-2-kinase/fructose-2,6-bisphosphatase 3 (PFKFB3), the method comprising contacting PFKFB3 with an effective amount of a compound of Formula (0), (I), (Ia), (Ib), (II), (III), (IV), (V), (VI), (VII), (VIIA) or (VIIB).

Also disclosed herein is a method of inhibiting 6-phosphofructo-2-kinase/fructose-2,6-bisphosphatase 4 (PFKFB4), the method comprising contacting PFKFB4 with an effective amount of a compound of Formula (0), (I), (Ia), (Ib), (II), (III), (IV), (V), (VI), (VII), (VIIA) or (VIIB).

Also disclosed herein is a method of inhibiting of PFKFB3 and/or PFKFB4 in a cell, the method comprising contacting a cell with an effective amount of a compound of Formula (0), (I), (Ia), (Ib), (II), (III), (IV), (V), (VI), (VII), (VIIA) or (VIIB).

Also disclosed herein is a method of treatment or prophylaxis of disease or condition for which glycolysis inhibition has beneficial effect, comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of treatment or prophylaxis of disease or condition for which PFKFB3 and/or PFKFB4 inhibition has beneficial effect, comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of reducing glycolytic flux in a cell, the method comprising contacting the cell with an effective amount of a compound of Formula (0), (I), (Ia), (Ib), (II), (III), (IV), (V), (VI), (VII), (VIIA) or (VIIB).

Also disclosed herein is a method of treating an autoimmune disease, an inflammatory disorder, a metabolic disease, a viral disease, a proliferative disease comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of reducing proliferative capacity in a cell, the method comprising contacting the cell with an effective amount of a compound of Formula (0), (I), (Ia), (Ib), (II), (III), (IV), (V), (VI), (VII), (VIIA) or (VIIB).

Also disclosed herein is a method of increasing of cell antioxidant capacity, the method comprising contacting the cell with an effective amount of a compound of Formula (0), (I), (Ia), (Ib), (II), (III), (IV), (V), (VI), (VII), (VIIA) or (VIIB).

Also disclosed herein is a method of enhancing the effect of radiation treatment of cancer, the method comprising administering to a subject in need thereof an effective amount of a compound of Formula (0), (1), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of decreasing the ability of the cancer cells to repair their DNA, the method comprising contacting the cell with an effective amount of a compound of Formula (0), (I), (Ia), (Ib), (II), (III), (IV), (V), (VI), (VII), (VIIA) or (VIIB).

Also disclosed herein is a method of sensitizing cancer cell towards cytostatic and/or radiation therapy, the method comprising contacting the cell with an effective amount of a compound of Formula (0), (I), (Ia), (Ib), (II), (III), (IV), (V), (VI), (VII), (VIIA) or (VIIB).

Also disclosed herein is a method of treatment of neoplasm sensitive to inhibition of PFKFB3 or/and PFKFB4, the method comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB)

Also disclosed herein is a method of treatment of neoplasm sensitive to inhibition of glycolysis, the method comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of reducing proliferative capacity in a cancer cell, the method comprising contacting the cancer cell with an effective amount of a compound of Formula (0), (I), (Ia), (Ib), (II), (III), (IV), (V), (VI), (VII), (VIIA) or (VIIB).

Also disclosed herein is a method of treatment of a cancer, the method comprising administering to the subject an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of treatment of cancer comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of treatment of solid tumor comprising administering to a subject in need thereof an effective amount of a compound of Formula (0), (I), (Ia), (Ib), (II), (III), (IV), (V), (VI), (VII), (VIIA) or (VIIB). or a pharmaceutical composition comprising a compound of Formula (0), (I), (Ia), (Ib), (II), (III), (IV), (V), (VI), (VII), (VIIA) or (VIIB).

Also disclosed herein is a method of treatment of a hematological cancer, comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of treatment of cancer selected from kidney cancer, colon cancer, pancreatic cancer, lung cancer, breast cancer, triple negative breast cancer, liver cancer, lymphoma, leukemia, myeloma, comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of treatment of cancer selected from: atypical teratoid rhabdoid tumor, brain tumor, anal cancer, astrocytoma, vaginal cancer, extrahepatic bile duct cancer, intraocular melanoma, hairy cell leukemia, hepatocellular liver cancer, gestational trophoblastic disease, germ cell tumor, hypopharyngeal cancer, histiocytosis, histiocytosis Langerhans, high-grade astrocytoma, astrocytoma, glioma, brain stem glioma, invasive lobular carcinoma, gastrointestinal carcinoid tumor, gastrointestinal stromal tumor, cutaneous T-cell lymphoma, peripheral T-cell lymphoma, non-specific lymphoma mantle cell, lymphogranulomatosis, colorectal cancer, craniopharyngioma, leukemia, mast cell leukemia, Burkitt's lymphoma, Hodgkin's lymphoma, Waldenström's macroglobulinemia (lymphoplasmacytic lymphoma), small bowel cancer, mastocytosis, malignant mesothelioma, melanoma, small-cell carcinoma (small-cell lung cancer), metastatic squamous neck cancer, myelodysplastic/myeloproliferative neoplasms, myelodysplastic syndrome, acute myeloid leukemia, chronic myelogenous leukemia, chronic myeloproliferative disease, multiple myeloma (plasma cell myeloma or Kahler's disease), male breast cancer, nasal cell carcinoma, neuroblastoma, non-small cell lung cancer, non-Hodgkin's lymphoma, Wilms tumor, osteosarcoma, malignant fibrous histiocytoma of bone, acute lymphoblastic leukemia, acute myeloid leukemia, papillomatosis, paraganglioma, parathyroid carcinoma, transitional cell cancer of the renal pelvis, transitional cell cancer of the ureter, pleuropulmonary blastoma, squamous cell carcinoma, renal cell carcinoma, ductal carcinoma in situ, rhabdomyosarcoma, vulvar cancer, eye cancer, head and neck cancer, throat cancer, laryngeal cancer, lip and oral cancer, stomach cancer, gall bladder cancer, bile duct cancer, skin cancer, cancer of the adrenal cortex, bone cancer, uterine cancer, Merkel carcinoma, bladder cancer, nasopharyngeal cancer, esophageal cancer, penile cancer, nasal cavity cancer, paranasal sinus cancer, renal pelvis cancer, ureter cancer, renal cancer, Papillary renal cell carcinoma, prostate cancer, rectal cancer, oral cancer, salivary gland cancer, cancer of the urethra, cancer of the cervix, thyroid cancer, endometrial cancer, cancer of the central nervous system, testis cancer, ovarian cancer, retinoblastoma, sarcoma, Kaposi's sarcoma, uterine sarcoma, soft tissue sarcoma, Ewing's sarcoma, cardiac tumor, Sezary syndrome, pharyngeal cancer, pheochromocytoma, fibrous histiocytoma of bone, chordoma, chronic myeloproliferative disorder, chronic lymphocytic leukemia, ependymoma, erythroleukemia, esthesioneuroblastoma, comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method for treating of a cancer, which comprises administering an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB) and at least one other anti-cancer medication.

Also disclosed herein is a method for treating of a cancer, which comprises administering an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB) and at least one other anti-cancer medication selected from Irinotecan and Sunitinib.

Also disclosed herein is a method for treating of a cancer, which comprises administering an effective amount of a compound of Formula (0), (I), (Ia), (Ib), (II), (III), (IV), (V), (VI), (VII), (VIIA) or (VIIB). or a pharmaceutical composition comprising a compound of Formula (0), (I), (Ia), (Ib), (II), (III), (IV), (V), (VI), (VII), (VIIA) or (VIIB). and at least one other anti-cancer medication, wherein anti-cancer medication is targeted therapy.

Also disclosed herein is a method for treating of a cancer, which comprises administering an effective amount of a compound of Formula (0), (I), (Ia), (Ib), (II), (III), (IV), (V), (VI), (VII), (VIIA) or (VIIB). or a pharmaceutical composition comprising a compound of Formula (0), (I), (Ia), (Ib), (II), (III), (IV), (V), (VI), (VII), (VIIA) or (VIIB). and at least one other anti-cancer medication, wherein anti-cancer medication is immunotherapy.

Also disclosed herein is a method of treating a cancer cell, comprising contacting the cancer cell with an effective amount of a compound of Formula (0), (I), (Ia), (Ib), (II), (III), (IV), (V), (VI), (VII), (VIIA) or (VIIB).

Also disclosed herein is a method of inducing an apoptosis of cancer cell, comprising contacting the cancer cell with an effective amount of a compound of Formula (0), (I), (Ia), (Ib), (II), (III), (IV), (V), (VI), (VII), (VIIA) or (VIIB).

Also disclosed herein is a method of inhibition of angiogenesis comprising administering to a subject in need thereof an effective amount of a compound of Formula (0), (I), (Ia), (Ib), (II), (III), (IV), (V), (VI), (VII), (VIIA) or (VIIB). or a pharmaceutical composition comprising a compound of Formula (0), (I), (Ia), (Ib), (II), (III), (IV), (V), (VI), (VII), (VIIA) or (VIIB).

Also disclosed herein is a method for neuroprotection comprising administering to a subject in need thereof an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of treatment of a neurodegenerative disease comprising administering to a subject in need thereof an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of treatment of a neurodegenerative disease selected from Alzheimer's disease, amyotrophic lateral sclerosis, Huntington's disease, and Parkinson's disease, Late-onset Alzheimer disease, stroke, ataxia telangiectasia (Louis-Bar syndrome), argyrophilic grain disease, autosomal dominant cerebellar ataxia, Batten disease (Spielmeyer-Vogt-Sjögren-Batten disease), corticobasal degeneration, corticobasal ganglionic degeneration, progressive supranuclear palsy (Steele-Richardson-Olszewski syndrome), Creutzfeldt-Jakob disease, fatal familial insomnia, frontotemporal dementia and parkinsonism linked to chromosome 17, neuronal intermediate filament inclusion disease, basophilic inclusion body disease, Pick disease, dementia with Lewy bodies, multiple-system atrophy, hereditary motor and sensory neuropathy with proximal dominance, infantile refsum disease, Machado-Joseph disease, mental retardation and microcephaly with pontine and cerebellar hypoplasia (mental retardation, X-linked, syndromic, Najm type), neuroacanthocytosis, pontocerebellar hypoplasia, pyruvate dehydrogenase deficiency (pyruvate dehydrogenase complex deficiency), refsum disease (heredopathia atactica polyneuritiformis), abetalipoproteinemia (Bassen-Kornzweig syndrome), frontotemporal lobar degeneration, spinal muscular atrophy, Friedreich's ataxia, spinocerebellar ataxia, dentatorubral-pallidoluysian atrophy, Gerstmann-Sträussler-Scheinker syndrome, motor neurone disease, Charcot disease or Lou Gehrig's disease, sclerosis, spinal muscular atrophy, depression, bipolar disorder comprising administering to a subject in need thereof an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of decreasing a glycolytic uptake in neuron, comprising contacting the neuron with an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of prevention of apoptotic death of neuron, comprising contacting the neuron with an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of prevention of apoptotic death of neuron triggered by glutamate receptor over-activation, administering to a subject in need thereof an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising a PFKFB3 inhibitor, including but not limited to a compound of Formula (0), (I), (Ia), (Ib), (II), (Ill), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of prevention of apoptotic death of neuron triggered by glutamate receptor over-activation, comprising contacting the neuron with an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of decreasing a glycolytic uptake in astrocyte, comprising contacting the astrocyte with an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of inhibition reactive astrocyte proliferation comprising administering to a subject in need thereof an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising a PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of protection of neuron against excitotoxicity comprising administering to a subject in need thereof an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising a PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of protection of enteric neuron against excitotoxicity comprising administering to a subject in need thereof an effective amount of PFKFB3 inhibitor, including but not limited to a compound of Formula (0), (I), (la), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising a PFKFB3 inhibitor, including but not limited to a compound of Formula (0), (I), (la), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of protection of neuron against excitotoxicity comprising, comprising contacting the neuron with an effective amount of a PFKFB3 inhibitor, including but not limited to a compound of Formula (0), (I), (la), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of treatment of an autoimmune disease comprising administering to a subject in need thereof an effective amount of a compound of Formula (0), (I), (la), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of treatment of an autoimmune disease selected from psoriasis, systemic lupus erythematosus, scleroderma, graft-versus-host disease, or transplanted organ rejection comprising administering to a subject in need thereof an effective amount of a compound of Formula (0), (I), (la), (Ib), (II), (III), (IV), (V), (VI), (VII), (VIIA) or (VIIB) or a pharmaceutical composition comprising a compound of Formula (0), (I), (la), (Ib), (II), (III), (IV), (V), (VI), (VII), (VIIA) or (VIIB).

Also disclosed herein is a method of treatment inflammation, comprising administering to a subject in need thereof an effective amount of a compound of Formula (0), (I), (la), (Ib), (II), (III), (IV), (V), (VI), (VII), (VIIA) or (VIIB) or a pharmaceutical composition comprising a compound of Formula (0), (I), (la), (Ib), (II), (III), (IV), (V), (VI), (VII), (VIIA) or (VIIB).

Also disclosed herein is a method of treatment of disorder selected from atherosclerosis, arthritis, rheumatoid arthritis, cystic fibrosis, inflammatory bowel disease, cerebral ischemia, neurological insult, influenza, inflammation, comprising administering to a subject in need thereof an effective amount of a PFKFB3 inhibitor, including but not limited to a compound of Formula (0), (I), (la), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising a PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of decreasing atherosclerotic inflammation and/or at least one of its clinical consequences comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB)

Also disclosed herein is a method of treatment of metabolic disease comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of treatment of glucose metabolism disorder comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of treatment of hyperlactatemia comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of immunosuppression, comprising the step of administering to a patient in need thereof a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of cancer, an autoimmune disease, an inflammatory disorder, a metabolic disease, a viral disease, a proliferative disease comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of neoplasm sensitive to inhibition of PFKFB3 or/and PFKFB4, the method comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB)

Also disclosed herein is a method of prophylaxis of neoplasm sensitive to inhibition of glycolysis, the method comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of a cancer, the method comprising administering to the subject an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of cancer selected from solid tumors, namely kidney, colon, pancreas, lung, breast and liver cancers, and hematologic neoplasms, namely lymphoma, leukemia and myeloma, a hematological cancer, breast cancer, comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of a hematological cancer, comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of cancer selected from kidney cancer, colon cancer, pancreatic cancer, lung cancer, breast cancer, triple negative breast cancer, liver cancer, lymphoma, leukemia, myeloma, comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of cancer selected from: atypical teratoid rhabdoid tumor, anal cancer, astrocytoma, vaginal cancer, extrahepatic bile duct cancer, intraocular melanoma, hairy cell leukemia, hepatocellular liver cancer, gestational trophoblastic disease, germ cell tumor, hypopharyngeal cancer, histiocytosis, histiocytosis Langerhans, glioma, high-grade astrocytoma, astrocytoma, brain stem glioma, invasive lobular carcinoma, gastrointestinal carcinoid tumor, gastrointestinal stromal tumor, cutaneous T-cell lymphoma, peripheral T-cell lymphoma, non-specific lymphoma mantle cell, lymphogranulomatosis, colorectal cancer, craniopharyngioma, leukemia, mast cell leukemia, Burkitt's lymphoma, Hodgkin's lymphoma, Waldenström's macroglobulinemia (lymphoplasmacytic lymphoma), small bowel cancer, mastocytosis, malignant mesothelioma, melanoma, small-cell carcinoma (small-cell lung cancer), metastatic squamous neck cancer, myelodysplastic/myeloproliferative neoplasms, myelodysplastic syndrome, acute myeloid leukemia, chronic myelogenous leukemia, chronic myeloproliferative disease, multiple myeloma (plasma cell myeloma or Kahler's disease), male breast cancer, nasal cell carcinoma, neuroblastoma, non-small cell lung cancer, non-Hodgkin's lymphoma, Wilms tumor, osteosarcoma, malignant fibrous histiocytoma of bone, acute lymphoblastic leukemia, acute myeloid leukemia, papillomatosis, paraganglioma, parathyroid carcinoma, transitional cell cancer of the renal pelvis, transitional cell cancer of the ureter, pleuropulmonary blastoma, squamous cell carcinoma, renal cell carcinoma, ductal carcinoma in situ, rhabdomyosarcoma, vulvar cancer, eye cancer, head and neck cancer, throat cancer, laryngeal cancer, lip and oral cancer, stomach cancer, gall bladder cancer, bile duct cancer, skin cancer, cancer of the adrenal cortex, bone cancer, uterine cancer, Merkel carcinoma, bladder cancer, nasopharyngeal cancer, esophageal cancer, penile cancer, nasal cavity cancer, paranasal sinus cancer, renal pelvis cancer, ureter cancer, renal cancer, Papillary renal cell carcinoma, prostate cancer, rectal cancer, oral cancer, salivary gland cancer, cancer of the urethra, cancer of the cervix, thyroid cancer, endometrial cancer, cancer of the central nervous system, testis cancer, ovarian cancer, retinoblastoma, sarcoma, Kaposi's sarcoma, uterine sarcoma, soft tissue sarcoma, Ewing's sarcoma, cardiac tumor, Sezary syndrome, pharyngeal cancer, pheochromocytoma, fibrous histiocytoma of bone, chordoma, chronic myeloproliferative disorder, chronic lymphocytic leukemia, ependymoma, erythroleukemia, and esthesioneuroblastoma, comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method for prophylaxis of a cancer, which comprises administering an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of a neurodegenerative disease comprising administering to a subject in need thereof an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising a PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of prophylaxis of a neurodegenerative disease selected from Alzheimer's disease, amyotrophic lateral sclerosis, stroke, Huntington's disease, and Parkinson's disease, Late-onset Alzheimer disease, ataxia telangiectasia (Louis-Bar syndrome), argyrophilic grain disease, autosomal dominant cerebellar ataxia, Batten disease (Spielmeyer-Vogt-Sjögren-Batten disease), corticobasal degeneration, corticobasal ganglionic degeneration, progressive supranuclear palsy (Steele-Richardson-Olszewski syndrome), Creutzfeldt-Jakob disease, fatal familial insomnia, frontotemporal dementia and parkinsonism linked to chromosome 17, neuronal intermediate filament inclusion disease, basophilic inclusion body disease, Pick disease, dementia with Lewy bodies, multiple-system atrophy, hereditary motor and sensory neuropathy with proximal dominance, infantile refsum disease, Machado-Joseph disease, mental retardation and microcephaly with pontine and cerebellar hypoplasia (mental retardation, X-linked, syndromic, Najm type), neuroacanthocytosis, pontocerebellar hypoplasia, pyruvate dehydrogenase deficiency (pyruvate dehydrogenase complex deficiency), refsum disease (heredopathia atactica polyneuritiformis), abetalipoproteinemia (Bassen-Kornzweig syndrome), frontotemporal lobar degeneration, spinal muscular atrophy, Friedreich's ataxia, spinocerebellar ataxia, dentatorubral-pallidoluysian atrophy, Gerstmann-Sträussler-Scheinker syndrome, motor neurone disease, Charcot disease or Lou Gehrig's disease, sclerosis, spinal muscular atrophy, depression, and bipolar disorder comprising administering to a subject in need thereof an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising a PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of prophylaxis of an autoimmune disease comprising administering to a subject in need thereof an effective amount of a compound of Formula (0), (I), (Ia), (Ib), (II), (III), (IV), (V), (VI), (VII), (VIIA) or (VIIB). or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of a disease selected from psoriasis, systemic lupus erythematosus, scleroderma, graft-versus-host disease, and transplanted organ rejection comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of an inflammatory disorder comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of disorder selected from atherosclerosis, arthritis, rheumatoid arthritis, cystic fibrosis, inflammatory bowel disease, cerebral ischemia, neurological insult, influenza, inflammation, comprising administering to a subject in need thereof an effective amount of PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application or a pharmaceutical composition comprising a compound of PFKFB3 inhibitor, including but not limited to a compound of Formula (0), (I), (Ia), (Ib), (II), (Ill), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application.

Also disclosed herein is a method of prophylaxis of metabolic disease comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of glucose metabolism disorder comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of prophylaxis of hyperlactatemia comprising administering to a subject in need thereof an effective amount of a compound of Formula (0), (I), (Ia), (Ib), (II), (III), (IV), (V), (VI), (VII), (VIIA) or (VIIB) or a pharmaceutical composition comprising a compound of Formula (0), (I), (Ia), (Ib), (II), (III), (IV), (V), (VI), (VII), (VIIA) or (VIIB).

Also disclosed herein is a method of manufacturing a medication, comprising the use of a compound of Formula (0), (I), (Ia), (Ib), (II), (III), (IV), (V), (VI), (VII), (VIIA) or (VIIB) as an active ingredient.

Also disclosed herein is a method of manufacturing a medication, comprising the use of a compound of Formula (0), (I), (Ia), (Ib), (II), (III), (IV), (V), (VI), (VII), (VIIA) or (VIIB) as an active ingredient, wherein the medicament is at least one of the following: medicament for treating a disease or condition for which glycolysis inhibition has beneficial effect, medicament for treating a cancer, a neuroprotector, a medicament for the treatment or prophylaxis of a disease or condition for which inhibition of kinase activity of PFKFB3 and/or PFKFB4 has beneficial effect, an inhibitor of glycolysis, an inhibitor of angiogenesis.

Also disclosed herein is a kit for treating a PFKFB3 and/or PFKFB4-mediated condition, comprising (a) a pharmaceutical composition comprising a compound of Formula (0), (I), (Ia), (Ib), (II), (III), (IV), (V), (VI), (VII), (VIIA) or (VIIB); and (b) instructions for use.

Also disclosed herein is a kit for treating a cancer, comprising (a) a pharmaceutical composition comprising a compound of Formula (0), (I), (Ia), (Ib), (II), (III), (IV), (V), (VI), (VII), (VIIA) or (VIIB); and (b) instructions for use.

In some embodiments, the compound described in this disclosure or a pharmaceutically acceptable salt thereof is in the form of a prodrug. In some embodiments, the prodrug comprises an ester moiety. In some embodiments the prodrug comprises an amide moiety.

Also disclosed herein is a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application and one or more pharmaceutically acceptable carrier comprised in the inventions described in any one of the items 1548 to 1848. Also disclosed herein is a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application in combination with another therapeutic agent, and optionally, one or more pharmaceutically acceptable carriers. In some embodiments, the pharmaceutical composition further comprises a second therapeutic agent.

Also disclosed herein is a method of modulating the activity of PFKFB3 in a neuron, comprising contacting the neuron with an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application.

Also disclosed herein is a method for neuroprotection comprising administering to a subject in need thereof an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application.

Also disclosed herein is a method of treatment of a neurodegenerative disease comprising administering to a subject in need thereof an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application.

Also disclosed herein is a method of treatment of a neurodegenerative disease selected from Alzheimer's disease, amyotrophic lateral sclerosis, Huntington's disease, and Parkinson's disease, Late-onset Alzheimer disease, stroke, ataxia telangiectasia (Louis-Bar syndrome), argyrophilic grain disease, autosomal dominant cerebellar ataxia, Batten disease (Spielmeyer-Vogt-Sjögren-Batten disease), corticobasal degeneration, corticobasal ganglionic degeneration, progressive supranuclear palsy (Steele-Richardson-Olszewski syndrome), Creutzfeldt-Jakob disease, fatal familial insomnia, frontotemporal dementia and parkinsonism linked to chromosome 17, neuronal intermediate filament inclusion disease, basophilic inclusion body disease, Pick disease, dementia with Lewy bodies, multiple-system atrophy, hereditary motor and sensory neuropathy with proximal dominance, infantile refsum disease, Machado-Joseph disease, mental retardation and microcephaly with pontine and cerebellar hypoplasia (mental retardation, X-linked, syndromic, Najm type), neuroacanthocytosis, pontocerebellar hypoplasia, pyruvate dehydrogenase deficiency (pyruvate dehydrogenase complex deficiency), refsum disease (heredopathia atactica polyneuritiformis), abetalipoproteinemia (Bassen-Kornzweig syndrome), frontotemporal lobar degeneration, spinal muscular atrophy, Friedreich's ataxia, spinocerebellar ataxia, dentatorubral-pallidoluysian atrophy, Gerstmann-Sträussler-Scheinker syndrome, motor neurone disease, Charcot disease or Lou Gehrig's disease, sclerosis, spinal muscular atrophy, depression, bipolar disorder comprising administering to a subject in need thereof an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application.

Also disclosed herein is a method of decreasing a glycolytic uptake in neuron, comprising contacting the neuron with an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application.

Also disclosed herein is a method of prevention of apoptotic death of neuron, comprising contacting the neuron with an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described below in this application or other PFKFB3 inhibitors described in this application

Also disclosed herein is a method of prevention of apoptotic death of neuron triggered by glutamate receptor over-activation, administering to a subject in need thereof an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or a pharmaceutical composition comprising a compound of Formula (0), (I), (Ia), (Ib), (II), (Ill), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of prevention of apoptotic death of neuron triggered by glutamate receptor over-activation, comprising contacting the neuron with an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of decreasing a glycolytic uptake in astrocyte, comprising contacting the astrocyte with an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of inhibition reactive astrocyte proliferation comprising administering to a subject in need thereof an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of protection of neuron against excitotoxicity comprising administering to a subject in need thereof an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of protection of enteric neuron against excitotoxicity comprising administering to a subject in need thereof an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of protection of neuron against excitotoxicity comprising, comprising contacting the neuron with an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of treatment of an autoimmune disease comprising administering to a subject in need thereof an effective amount of a compound of Formula (0), (I), (Ia), (Ib), (II), (Ill), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of prophylaxis of a neurodegenerative disease comprising administering to a subject in need thereof an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of prophylaxis of a neurodegenerative disease selected from Alzheimer's disease, amyotrophic lateral sclerosis, stroke, Huntington's disease, and Parkinson's disease, Late-onset Alzheimer disease, ataxia telangiectasia (Louis-Bar syndrome), argyrophilic grain disease, autosomal dominant cerebellar ataxia, Batten disease (Spielmeyer-Vogt-Sjögren-Batten disease), corticobasal degeneration, corticobasal ganglionic degeneration, progressive supranuclear palsy (Steele-Richardson-Olszewski syndrome), Creutzfeldt-Jakob disease, fatal familial insomnia, frontotemporal dementia and parkinsonism linked to chromosome 17, neuronal intermediate filament inclusion disease, basophilic inclusion body disease, Pick disease, dementia with Lewy bodies, multiple-system atrophy, hereditary motor and sensory neuropathy with proximal dominance, infantile refsum disease, Machado-Joseph disease, mental retardation and microcephaly with pontine and cerebellar hypoplasia (mental retardation, X-linked, syndromic, Najm type), neuroacanthocytosis, pontocerebellar hypoplasia, pyruvate dehydrogenase deficiency (pyruvate dehydrogenase complex deficiency), refsum disease (heredopathia atactica polyneuritiformis), abetalipoproteinemia (Bassen-Kornzweig syndrome), frontotemporal lobar degeneration, spinal muscular atrophy, Friedreich's ataxia, spinocerebellar ataxia, dentatorubral-pallidoluysian atrophy, Gerstmann-Sträussler-Scheinker syndrome, motor neurone disease, Charcot disease or Lou Gehrig's disease, sclerosis, spinal muscular atrophy, depression, and bipolar disorder comprising administering to a subject in need thereof an effective amount of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or a pharmaceutical composition comprising a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below

Also disclosed herein is a method of manufacturing a medication, comprising the use of a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below as an active ingredient, wherein the medicament is at least one of the following: a neuroprotector, a anti-aging medication, a rejuvenation medication.

Also disclosed herein is a kit for treating a PFKFB3-mediated neurogenerative condition, comprising (a) a pharmaceutical composition comprising a PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application; and (b) instructions for use.

Also disclosed herein is a kit for treating a PFKFB3-mediated aging related disease or condition, comprising (a) a pharmaceutical composition comprising a PFKFB3 inhibitor, including but not limited to a compound of Formula (0), (I), (Ia), (Ib), (II), (III), (IV), (V), (VI), (VII), (VIIA), (VIIB), (VIII), (IX), (X), (XI), (XII), (XIII) or any one of PFKFB3 inhibitors selected from any one of the items 1 to 1863 or items A,B,C,D,E, F, G,H below or other formulas described in this application or other PFKFB3 inhibitors described in this application; and (b) instructions for use.

In some embodiments this invention is a kit, comprising an agent, disclosed or described in this disclosure, including but not limited to PFKFB3 inhibitor, or composition disclosed in this application or its functional or structural analog or prodrug and the notice, description or instruction regarding the reduction or modulating or binding or inhibiting or degrading of PFKFB3, by the means of such agent or composition for anti-aging treatment or for neuroprotection, optionally comprising at least the medication labeling information, optionally wherein such notice, description or instruction comprises information about administration of corresponding agent or composition in a dosage and regimen, optionally to produce the biological effect comparable or alike or close to the inhibition of PFKFB3.

In some embodiments this invention is a kit, comprising an agent, modulating or binding or inhibiting or degrading or activating at least one of the Indirect Targets, optionally, wherein such modulation or binding or inhibiting or degrading or activating has anti-aging or neuroprotective effect and the notice, description or instruction regarding the modulation or binding or inhibiting or degrading or activating or reduction of at least one such Indirect Targets, by the means of such agent or composition for anti-aging treatment or neuroprotection, optionally comprising at least the medication labeling information, optionally wherein such notice, description or instruction comprises information about administration of corresponding agent or composition in dosage and regimen to produce the biological effect comparable or alike or close to the inhibition of PFKFB3.

In some embodiments, this invention is a kit, comprising the PFKFB3 inhibitor or pharmaceutical composition, comprising such inhibitor and a notice, description or instruction regarding the inhibition of PFKFB3 by the means of such inhibitor or pharmaceutical composition for anti-aging treatment.

In some embodiments, this invention is a kit, comprising an PFKFB3 inhibitor or any other agent of this invention and a notice, description or instruction for its use by human or by other animal subject in a dosage and regimen to maintain concentration of such agent in blood of such subject. In some embodiments such notice, description or instruction comprises information related to the deactivation, inhibition or of PFKFB3 for anti-aging treatment.

In some embodiments, this invention is a kit, comprising an PFKFB3 inhibitor or any other agent of this invention and a notice, description or instruction for its use by human or by other animal subject in a dosage and regimen to maintain concentration of such agent in blood of such subject. In some embodiments such notice, description or instruction comprises information related to the deactivation, inhibition or of PFKFB3 for neuroprotection.

In some embodiments, the mentioned notice, description or instruction attached to such device or imprinted or drawn or in any other way displayed on such device or in any other way associated with such kit or composition (e.g. in machine readable form). One of the primary purposes of some aspects of his invention is to provide medication for anti-aging treatment and treatment of neurodegeneration. As a rule the medication or kit comprising medication of this invention should be accompanied with the notice, description or instruction (e.g., treatment and/or operation guidelines).

In some embodiments the contents and appearance of such notice, description or instruction is regulated by the respective national or international rules regarding labeling of medication, incorporated here by reference or such notice, description or instruction comprise at least part or optionally most of the or optionally all the information required by applicable medicines labeling regulations. For example, The Federal Food, Drug and Cosmetic Act (FFDCA) in USA is the law under which the FDA takes action against regulated products. In some embodiments ‘label’ is defined as a: ‘display of written, printed, or graphic matter upon the immediate container of any article . . . ‘The term’ immediate container’ does not include package liners. In some embodiments ‘labeling’ is: ‘all labels and other written, printed, or graphic matter (1) upon any article or any of its containers or wrappers, or (2) accompanying such article’ at any time while a device is held for sale after shipment or delivery for shipment in interstate commerce. The term ‘accompanying’ is interpreted liberally to mean more than physical association with the product. It extends to posters, tags, pamphlets, circulars, booklets, brochures, instruction books, direction sheets, fillers, etc. ‘Accompanying’ also includes labeling that is brought together with the device after shipment or delivery for shipment in interstate commerce. According to an appellate court decision: “Most, if not all advertising, is labeling.

The notice, description or instruction, including but not limited to labeling means (e.g., treatment and/or operation guidelines) can be provided in any form that conveys the requisite information. Instruction means can be audio, for example spoken word, recorded in analog or digital form (e.g., audio recording), or received and/or transmitted in analog or digital form (e.g., by telephone, conference call, or audio signal transmitted over a network). Such information can also be visual or video, for example hard-copy (e.g., as a manual, recorded medium, booklet, leaflet, book and the like) or soft-copy (e.g., recorded in analog or digital form as a file recorded on an magnit, electronic, optical, or computer readable medium such as a DVD, disk drive, CD-ROM and the like). Additionally, instruction means can be interactive or real-time (e.g., a teleconference or internet chat or chat bot).

Some mediums, kits, or agents of this invention can include printed or made in any other way instructions to inform the user of the steps required to properly use it, optionally for reduction, deactivation, inhibition or degradation of PFKFB3 for anti-aging treatment or for neuroprotection.

In some embodiments, an mediums, kits or agents of this invention include a label configured to be coupled to respective mediums, kits or agents of this invention. The label includes a first surface and a second surface. In some embodiments, the first surface can be coupled to an outer surface of mediums, kits or agents of this invention. In some embodiments, for example, the first surface can include an adhesive. The second surface can include a textual indicia, such as, for example, a description of the mediums, kits, or agents of this invention, a mark indicating its manufacturer or distributor and/or an instruction associated with the use of such mediums, kits, or agents of this invention. The label can further include an electronic circuit system configured to output an electronic signal. In some embodiments, the electronic signal can include an instruction associated with the use of the mediums, kits or agents of this invention.

In some embodiments the instruction is an instruction for use as medication.

In some embodiments, the notice, description or instruction, including but not limited to labeling can be shown on the lenses, computer glasses, transmitted via brain computer interface or by any other means or can be encoded by the Quick Response Code or any other machine readable form.

The notice, description or instruction, including but not limited to labeling can be implemented in digital electronic circuitry, or in computer firmware, hardware, software, or in combinations thereof. The implementation can be as a computer program product, e.g., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be recorded in any form of programming language, including compiled or interpreted languages, and the computer program can be deployed in any form, including as a stand-alone program or as a subroutine, element, or other unit suitable for use in a computing environment. A computer program can be deployed to be executed on one computer or on multiple computers at one site or several sites.

The notice, description or instruction, including but not limited to labeling can be performed by one or more programmable processors executing a computer program to perform functions of the invention by operating on input data and generating output. It can also be performed by, and an apparatus can be implemented as, special purpose logic circuitry, e.g., an FPGA (field programmable gate array) or an ASIC (application-specific integrated circuit). Subroutines can refer to portions of the computer program and/or the processor/special circuitry that implements that functionality.

In some embodiments, kit of this invention further comprises information about approval by the relevant agency of manufacture, use or sale for human administration.

The non-limiting examples of kits of this invention, could be paper kits which are the paper boxes, comprising corresponding pharmaceutical described in this disclosure, paper instruction and description, comprising name of intervention, indication and instruction.

| | | |
|---|---|---|
| Compound CHEMBL3422676 | Anti-aging, rejuvenation, anti-frailty, | For IV injection, one vial per |
| (AZ67) | neuroprotection, amelioration of | injection, once a day, 4 weeks. |
| | cognitive decline, improvement of | |
| | hands grip force, amelioration of | |
| | other aging related declines. | |
| Other PFKFB3 inhibitor, e.g. 4-({4- | Anti-aging, rejuvenation, anti-frailty, | For IV injection, one vial per |
| carboxy-2′,4′-dichloro-[1,1′- | neuroprotection, amelioration of | injection, two times a day, 12 |
| biphenyl]-3-yl}carbamoyl)-6- | cognitive decline, improvement of | weeks. |
| hydroxybenzene-1,3-dicarboxylic | hands grip force, amelioration of | |
| acid | other aging related declines. | |
| Compound CHEMBL3422676 | Neuroprotector, Cerebral ischemia, | For IV injection, one vial per |
| (AZ67) | including ischemic stroke | injection, once a day, 4 weeks. |
| Other PFKFB3 inhibitor, , e.g. 4-({4- | Neuroprotector, Cerebral ischemia, | For IV injection, one vial per |
| carboxy-2′,4′-dichloro-[1,1′- | including ischemic stroke | injection, two times a day, 12 weeks |
| biphenyl]-3-yl}carbamoyl)-6- | | |
| hydroxybenzene-1,3-dicarboxylic | | |
| acid | | |
| Compound CHEMBL3422676 | Neuroprotector, traumatic brain | For stereotactic injection, or internal |
| (AZ67) | injury, Cerebral ischemia, including | carotid artery, or lateral ventricle |
| | ischemic stroke | delivery or direct brain infusion. |
| | | One vial per injection/infusion. |
| Compound CHEMBL3422676 | Neuroprotector, stroke, Cerebral | IV Administered via jugular vein |
| (AZ67) | ischemia, including ischemic stroke | right after reperfusion |
| | | One vial per injection. |

### Method of Testing Efficacy

In some embodiments this invention is a method, including but not limited to method of testing of efficacy of therapy deleting, reducing, binding, inhibiting or degrading PFKFB3 or therapy modulating (by deleting, reducing, binding, deactivating, inhibiting or degrading or by activating or by any other way) at least one of Indirect Targets, wherein such modulation has an anti-aging or neuroprotective effect, comprising the checking in the subject treated by such therapy at least one of the following: checking biological age of the patient, at least one aging biomarker, at least one of markers of neurodegeneration or neuroprotection, at least one age related deficit or disease, at least one of rejuvenation marker, frailty, health span or life span, or any other marker or parameter reasonable for checking in testing of anti-aging therapy efficacy, optionally wherein therapy is a monoclonal or polyclonal antibody, optionally humanized, which recognizes the receptor of at least one respective Indirect Targets, protein, aptamer, peptide, polymer, virus or small molecule, binding or inhibiting or degrading PFKFB3 or at least one of Indirect Targets or any molecule or composition described in this disclosure or its analog.

In some embodiments checking of efficacy of therapy can be done as measurement of markers or symptoms of related diseases or conditions which is conducted in 1 month after the administration of therapy in therapeutically effective amount, in 3 months, in 6 months, in 12 months, in 18 months, in 24 months or in 36 months after such infusion, or in around such date, or in date reasonably defined by the doctor based on the parameter being measured and other factors known to the expert in the field.

### Related Systems

In some embodiments this invention is a tangible medium comprising a computer program, which, when executed, causes a medium to perform a method comprising: attribution to the information about a subject an information about a treatment or therapy related to deactivating, deleting, reducing, binding, inhibiting or degrading PFKFB3 or in some embodiments to treatment or therapy related to modulating or binding or inhibiting or degrading or activating at least one of Indirect Targets, wherein such modulation or binding or inhibiting or degrading or activating has anti-aging or neuroprotective effect, optionally wherein treatment is anti-aging or neuroprotective treatment, optionally wherein such deactivating, deleting, reducing, binding, inhibiting or degrading is achieved by composition or agent described in this disclosure, optionally further comprising attributing to the information about patient before or after or before and after the treatment to information about checking of at least one selected from the group: biological age of the patient, at least one aging biomarker, at least one age related deficit or disease, at least one of rejuvenation marker, frailty, health span or life span, neuroprotection or neurodegeneration level or marker.

An example of such tangible medium could be a APPLE™ 2014 MACBOOK AIR™ 13″ Intel™ i5 with Microsoft™ Excel™ installed and executed on it, wherein to patient with name John Junior Smith (born 2 Jan. 1937) the information about inhibition of PFKFB3 is attributed in the sense that is logically linked as an information in Excel table (in this example attribution is realized as placing the information about treatment by inhibition of PFKFB3 in the same line in the file with the name and ID of the patient to whom such treatment is prescribed) and allows easy finding of patients in need of anti-aging or neuroprotective treatment to whom such treatment is prescribed and other processing of such information.

| | | Date of | | Treatment |
|---|---|---|---|---|
| patient ID | Name | birth | diagnosis | prescribed |
| 28282838 | John | 3 Jan. | Before treatment: moderate | Administration |
| | Junior | 1937 | frailty, moderate cognitive | of PFKFB3 |
| | Smith | | decline, hand grip strength −21.3 | inhibitor. |
| | | | kg and other age related deficites | |
| 28282839 | John | 3 Aug. | Before treatment: neurodegeneration | Administration |
| | Black | 1940 | | of PFKFB3 |
| | | | | inhibitor |

One of the examples of such PFKFB3 inhibitors could be a pharmaceutical composition comprising compound CHEMBL3422676 (AZ67), another example can be 4-({4-carboxy-2′,4′-dichloro-[1,1′-biphenyl]-3-yl}carbamoyl)-6-hydroxybenzene-1,3-dicarboxylic acid.

Processors suitable for the execution of a computer program related to this invention include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer also includes, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

In some embodiments this invention is a tangible medium comprising a computer program, which, when executed, causes a device to perform a method comprising: attribution to the information regarding therapeutic agent or composition an information about deactivating, deleting, reducing, binding, inhibiting or degrading of PFKFB3, or in some embodiments an information about modulating or binding or inhibiting or degrading or activating at least one of Indirect Targets, if such modulation or binding or inhibiting or degrading or activating has anti-aging or neuroprotective effect or the purpose of such action is to induce anti-aging effect or neuroprotective effect, optionally, wherein information about deleting, reducing, binding, inhibiting or degrading PFKFB3 is associated with the information about anti-aging or neurodegeneration treatment, optionally wherein agent is described in this disclosure.

In some embodiments this invention is a tangible medium comprising a computer program, which, when executed, causes a medium to perform a method comprising: attribution to the information about a therapy, agent, composition, medium or procedure associated with deletion, reduction, binding, inhibiting or degrading of PFKFB3 to the information related to anti-aging treatment or to neuroprotection.

As an example of such attribution the excel file executed on the same computer as described above or website or webpage available in Internet hosted on the server e.g. www.ipage.com, can be suggested, any of which when executed show at list one line of the following:

| Name | Agent/Mechanism of Action/Mode of action | Indication |
|---|---|---|
| ASD1 | Small molecule PFKFB3 inhibitor, e.g. 4-({4- | anti-aging, anti-frailty, amelioration of moderate |
| | carboxy-2′,4′-dichloro-[1,1′-biphenyl]-3- | cognitive decline, amelioration hand grip strength |
| | yl}carbamoyl)-6-hydroxybenzene-1,3- | lose and amelioration of other age related deficits |
| | dicarboxylic acid | |
| GER23 | Small molecule PFKFB3 inhibitor | Neuroprotection, treatment of neurodegenerative |
| | | disease |
| GER56 | Compound CHEMBL3422676 (AZ67) | anti-aging, anti-frailty, amelioration of moderate |
| | | cognitive decline, amelioration hand grip strength |
| | | lose and amelioration of other age related deficits |
| FDS89 | Compound CHEMBL3422676 (AZ67) | Neuroprotection, treatment of neurodegenerative |
| | | disease |
| GERO288 | 4-({4-carboxy-2′,4′-dichloro-[1,1′-biphenyl]-3- | cancer |
| | yl}carbamoyl)-6-hydroxybenzene-1,3- | |
| | dicarboxylic acid or any other compound of of | |
| | Formula (0), (I), (Ia), (Ib), (II), (III), (IV), (V), (VI), | |
| | (VII), (VIIA), (VIIB). | |

In some embodiments this invention is a method, comprising an attribution to information about the patient an information about the treatment related to deactivating deleting, reducing, binding, inhibiting or degrading of PFKFB3 or related to information about the modulating or binding or inhibiting or degrading or activating at least one the Indirect Targets, wherein such modulation or binding or inhibiting or degrading or activating has anti-aging or neuroprotective effect, wherein such attribution is performed in database or medium, comprising a computer program, which, when executed, causes a medium to perform such attribution or in other medium, optionally wherein the treatment is described as administration of PFKFB3 inhibitor or pharmaceutical composition, comprising such inhibitor or administration of Indirect Target modulator or pharmaceutical composition, comprising such modulator.

In some embodiments this invention is a method, comprising an attribution of information about the patient to an information about the agent deactivating, deleting, reducing, binding, inhibiting or degrading at least one of PFKFB3 or about the agent modulating or binding or inhibiting or degrading or activating at least one at least one of the Indirect Targets, wherein such modulation or binding or inhibiting or degrading or activating has anti-aging or neuroprotective effect, optionally wherein agent is selected from the group: a monoclonal or polyclonal antibody, protein, aptamer, peptide, polymer, gene therapy, virus or small molecule, nanoparticle or any identification meaning such agent or composition, or to the information related to treatment associated with deletion, reduction, binding, inhibiting or degrading of PFKFB3, wherein such attribution is performed in database or medium, comprising a computer program, which, when executed, causes a medium to perform such attribution or in other medium, optionally wherein inhibiting or binding of PFKFB3 is achieved by at least one of the agent selected from the PFKFB3 inhibitors described in this application or is its analog.

In some embodiments this invention is a method, comprising attribution of information about the therapy, agent, medium or procedure associated with deactivation, deletion, reduction, binding, inhibiting or degrading of PFKFB3 to the information related to anti-aging treatment or neurodegeneration treatment, wherein such attribution is performed in database or medium, comprising a computer program, which, when executed, causes a medium to perform such attribution or in other medium, optionally to the labeling information related to medication.

In some embodiments this invention is a method of this disclosure, comprising attribution of information where in the patient age is above 30 years old or above 40 years old or above 50 years old and/or the patient is someone who is in need of anti-aging treatment and/or the patient is someone who is in need of neuroprotection treatment, optionally, wherein agent is selected from the group: a monoclonal or polyclonal antibody, optionally humanized, protein, aptamer, peptide, polymer, nanoparticle, virus or small molecule, or other agent described as PFKFB3 inhibitor in this application, or its analog or information about pharmaceutical composition, comprising such agent or its analog or any ID/identification meaning such agent or composition or wherein, wherein treatment is an anti-aging treatment or treatment of neurodegenerative disease or neuroprotection.

In some embodiments this invention is a method or a tangible medium comprising a computer program, which, when executed, causes a medium to perform a method comprising step of attributing to agent of this invention an information comprised in notice, description or instruction described in this disclosure for kits, comprising notice, description or instruction.

In some embodiments the method of this invention comprising attribution of information described in this disclosure is a computer implemented method. In some embodiments this invention is a method, the method of this invention, comprising attribution of information executed on the medium of this invention and described in corresponding part of this disclosure related to such medium.

In some embodiments this invention is a tangible medium or computer system or processor, comprising a executable instruction or computer program, which, when executed, causes a medium to perform a method comprising attribution of information described in this disclosure.

In some embodiments this invention is an apparatus to execute method described in this disclosure the apparatus comprising the processor comprising the tangible medium described in this disclosure.

SIRNA

In some embodiments PFKFB3 inhibitor is a PFKFB3 inhibiting Small RNA.

Accordingly in such embodiments kit, method, composition, pharmaceutical composition, use, PFKFB3 inhibitor, medium described in this application comprises instead of small molecule PFKFB3 inhibitor a PFKFB3 inhibiting Small RNA is used or PFKFB3 inhibitor is a PFKFB3 inhibiting Small RNA.

RNA interference (RNAi) is a natural process used by cells to regulate gene expression. The process to silence genes first begins with the entrance of a double-stranded RNA (dsRNA) molecule into the cell, which triggers the RNAi pathway. The double-stranded molecule is then cut into small double-stranded fragments by an enzyme called Dicer. These small fragments, which include small interfering RNAs (siRNA) and microRNA (miRNA), are approximately 21-23 nucleotides in length. The fragments integrate into a multi-subunit protein called the RNA-induced silencing complex, which contains Argonaute proteins that are essential components of the RNAi pathway. One strand of the molecule, called the “guide” strand, binds to RISC, while the other strand, known as the “passenger” strand is degraded. The guide or antisense strand of the fragment that remains bound to RISC directs the sequence-specific silencing of the target mRNA molecule. The genes can be silenced by siRNA molecules that cause the endonucleatic cleavage of the target mRNA molecules or by miRNA molecules that suppress translation of the mRNA molecule. With the cleavage or translational repression of the mRNA molecules, the genes that form them are essentially inactive. RNAi is thought to have evolved as a cellular defense mechanism against invaders, such as RNA viruses, or to combat the proliferation of transposons within a cell's DNA. Both RNA viruses and transposons can exist as double-stranded RNA and lead to the activation of RNAi. Currently, siRNAs are being widely used to suppress specific gene expression and to assess the function of genes. Companies utilizing this approach include Alnylam, Sanofi, Arrowhead, Discerna and Persomics, among others.

siRNAs can now be easily produced by the methods known in the art and modified to be used in vivo. Another option could be a purchase of siRNAs or siRNAs modified for in vivo use for respective targets.

For example Ambion® In Vivo siRNAs are designed using the Silencer® Select algorithm and incorporate chemical modifications that help provide superior serum stability for in vivo delivery.

Invitrogen™ Silencer™ Pre-designed siRNAs are available for all human, mouse, and rat gene targets in the RefSeq database. These siRNAs are designed for maximum potency and specificity using a highly effective and extensively tested algorithm. Each siRNA is synthesized to the highest quality standards and is provided with full sequence information. Furthermore, when one purchases three Silencer Pre-Designed siRNAs to the same target, there is a guarantee that with at least two of the siRNAs you will achieve >70% reduction in target mRNA levels.

The further modification and optimisation of siRNAs for human use is made by the methods known in the art.

Antisense therapy is a form of treatment. When the genetic sequence of a particular gene is known to be causative of a particular disease, it is possible to synthesize a strand of nucleic acid (DNA, RNA or a chemical analogue) that will bind to the messenger RNA (mRNA) produced by that gene and inactivate it, effectively turning that gene “off”. This is because mRNA has to be single stranded for it to be translated. Alternatively, the strand might be targeted to bind a splicing site on pre-mRNA and modify the exon content of an mRNA. Delivery Because nucleases that cleave the phosphodiester linkage in DNA are expressed in almost every cell, unmodified DNA molecules are generally degraded before they reach their targets. Therefore, antisense drug candidate molecules are generally modified during the drug discovery phase of their development. Additionally, most targets of antisense are located inside cells, and getting nucleic acids across cell membranes is also difficult. Therefore, most clinical candidates have modified DNA “backbones”, or the nucleobase or sugar moieties of the nucleotides are altered. Additionally, other molecules may be conjugated to antisense molecules in order to improve their ability to target certain cells or to cross barriers like cell membranes or the blood brain barrier

PFKFB3 inhibiting RNAi (siRNA, shRNA,miRNA) as well as non-viral DNA vectors can be delivered in vivo using a synthetic carrier for the siRNA or shRNA/DNA payload or naked DNA vectors or chemically modified siRNA (i.e. Ambion In Vivo siRNA). Synthetic carriers include cationic liposomes (stable nucleic-acid lipid particle SNALP carrier by Tekmira, siRNA-lipoplex AtuPLEX™), anionic liposome, polymeric carriers (cyclodextrin nanoparticles from Calando, biodegradable polymeric matrix LODER). For example, for systemic delivery of Ambion In Vivo siRNA (a dose starting with 7 mg/kg should be used) injection of siRNA solution of 0.7 mg/mL in PBS, saline (0.9% NaCl or variants containing sugars such as mannitol or glucose (5-15%) or Ringer's solution (147 mM NaCl, 4 mM KCl, 1.13 mM CaCl₂)) may be used. For hepatic delivery Invivofectamine 2.0 reagent (Invitrogen) may be used (˜3 mg/mL working solution). In order to prepare Invivofectamine-siRNA complex resuspended siRNA duplex should be diluted 1:1 Complexation Buffer. Then the solution should be added to an equal volume of warm Invivofectamine 2.0 Reagent, vortex for 2-3 seconds and incubated for 30 minutes at 50° C. Afterwards ˜14 volumes of PBS, pH 7.4 should be added. The solution is then diafiltrated using Amicon® Ultra-15 Centrifugal Device with Ultracel-50 membrane. The retentate retentate containing the Invivofectamine 2.0-siRNA complex is then collected, brought to 00 μL with PBS and used for in vivo injection immediately or alternatively can be stored at 4° C. for up to a week prior to injection. Specific silencing of targeted genes can be confirmed by the independent experiments known in the art.

Examples of commercially available siRNA for PFKFB3:

Ambion In Vivo siRNAs from ThermoFisher Scientific (https://www.thermofisher.com/ru/ru/home/life-science/rnai/introduction-to-in-vivo-rnai/ambion-in-vivo-sirna.html): s10359, s10357, s10358, n364686, n364691, n364684, n364683, n364689, n364690, n364685, n364687, n364682, n364688

MISSION® siRNA from Sigma Aldrich (https://www.sigmaaldrich.com/life-science/functional-genomics-and-rnai/)

SIHK1581 MISSION® siRNA Human Kinase PFKFB3 (siRNA2), Nano Scale 0.25 nmol

SIHK1580 MISSION® siRNA Human Kinase PFKFB3 (siRNA1), Nano Scale 0.25 nmol

SIHK1582 MISSION® siRNA Human Kinase PFKFB3 (siRNA3), Nano Scale 0.25 nmol

Examples of siRNA sequences for PFKFB3:

SEQ ID NO 1
GUCUUCGGUGUCUCCAUUAAU,
SEQ ID NO 2
GAAGCAGUACAGCUCCUACAA,
SEQ ID NO 3
GCAGUACAGCUCCUACAACUU,
SEQ ID NO 4
GCCUUAGCUGCCUUGAGAGAU,
SEQ ID NO 5
GAAGAGGAUCAGUUGCUAUGA,
SEQ ID NO 6
GACACCUACCCUGAGGAGUAU,
SEQ ID NO 7
GGGAGCAGGACAAGUACUAUU
SEQ ID NO 8
GGAGCAGGACAAGUACUAUUA,
SEQ ID NO 9
GCAGGAGAAUGUGCUGGUCAU,
SEQ ID NO 10
GCCUACUUCCUGGAUAAGAGU,
SEQ ID NO 11
GGAUAAGAGUGCAGAGGAGAU,
SEQ ID NO 12
GAGGUCAGAGGAUGCAAAGAA,
SEQ ID NO 13
GGAUGCAAAGAAGGGACCUAA

In some embodiments this invention is kit, method, composition, pharmaceutical composition, use, PFKFB3 inhibitor, medium of any one of items of this application, wherein PFKFB3 inhibitor is a small interfering RNA (siRNA) targeting a phosphoinositide PFKFB3 signal transduction pathway.

In some embodiments this invention is kit, method, composition, pharmaceutical composition, use, PFKFB3 inhibitor, medium of any one of items of this application, wherein PFKFB3 inhibitor is a small interfering RNA (siRNA) targeting a PFKFB3 signal transduction pathway

In some embodiments this invention relates to following siRNA items

1. In some embodiments this invention is kit, method, composition, pharmaceutical composition, use, PFKFB3 inhibitor, medium of any one of items of this application, wherein inhibitor of PFKFB3 is a chemically modified double stranded siRNA molecule that down regulates expression of an PFKFB3 gene via RNA interference (RNAi), wherein:

a) each strand of said siRNA molecule is independently about 18 to about 28 nucleotides in length; and

b) one strand of said siRNA molecule comprises nucleotide sequence having sufficient complementarity to an RNA of said PFKFB3 gene for the siRNA molecule to direct cleavage of said RNA via RNA interference.

2. The invention of item 1, wherein each strand of the siRNA molecule comprises about 18 to about 28 nucleotides, and wherein each strand comprises at least about 14 to 24 nucleotides that are complementary to the nucleotides of the other strand.

3. The invention of item 1, wherein said siRNA molecule is assembled from two separate oligonucleotide fragments wherein a first fragment comprises the sense strand and a second fragment comprises the antisense strand of said siRNA molecule.

4. The invention of item 3, wherein said sense strand is connected to the antisense strand via a linker molecule.

5. The invention of item 4, wherein said linker molecule is a polynucleotide linker.

6. The invention of item 4, wherein said linker molecule is a non-nucleotide linker.

7. The invention of item 3, wherein said second fragment comprises a terminal cap moiety at a 5′-end, a 3′-end, or both of the 5′ and 3′ ends of said second strand.

8. The invention of item 7, wherein said terminal cap moiety is an inverted deoxy abasic moiety.

9. The invention of item 3, wherein said first fragment comprises a phosphorothioate internucleotide linkage at the 3′ end of said first strand.

10. The invention of item 1, wherein said siRNA molecule comprises at least one 2′-sugar modification.

11. The invention of item 10, wherein said 2′-sugar modification is a 2′-deoxy-2′-fluoro modification.

12. The invention of item 10, wherein said 2′-sugar modification is a 2′—O-methyl modification.

13. The invention of item 10, wherein said 2′-sugar modification is a 2′-deoxy modification.

14. The invention of item 1, wherein said siRNA molecule comprises at least one nucleic acid base modification.

15. The invention of item 1, wherein said siRNA molecule comprises at least one phosphate backbone modification.

16. A composition comprising the siRNA molecule of any of the items of this application in a pharmaceutically acceptable carrier or diluent.

In some embodiments this invention relates to following siRNA items

1. In some embodiments this invention is kit, method, composition, pharmaceutical composition, use, PFKFB3 inhibitor, medium of any one of items of this application, wherein PFKFB3 inhibitor is an isolated siRNA (small interfering RNA) molecule comprising a sense region and an antisense region that down regulates expression of an PFKFB3 gene via RNA interference (RNAi), wherein the sense region comprises a nucleotide sequence selected from the group consisting of SEQ ID NOs: 1-, and wherein the antisense region comprises a sequence that is complementary to a nucleotide sequence from the group consisting of SEQ ID NOs: 1-.13

2. The invention of item 1, wherein the sense and antisense RNA strands forming the duplex region are covalently linked by a linker molecule.

3. The invention of item 1, wherein the siRNA further comprises non-nucleotide material.

4. The invention of item 1, wherein the linker molecule is a polynucleotide linker.

5. The invention of item 1, wherein the linker molecule is a non-nucleotide linker.

6. A recombinant nucleic acid construct comprising a nucleic acid that is capable of directing transcription of a small interfering RNA (siRNA), the nucleic acid comprising: (a) at least one promoter; (b) a DNA polynucleotide segment that is operably linked to the promoter, (c) a linker sequence comprising at least 4 nucleotides operably linked to the DNA polynucleotide segment of (b); and (d) operably linked to the linker sequence a second polynucleotide, wherein the polynucleotide segment of (b) comprises a polynucleotide that is selected from the group consisting of SEQ ID Nos: 1-13, wherein the second polynucleotide of (d) comprises a polynucleotide that is complementary to at least one polynucleotide from the group consisting of SEQ ID NOs: 1-13

RNAi may be introduced in vivo as virus-delivered shRNA, for example MISSION® In Vivo shRNA from Sigma Aldrich (https://www.sigmaaldrich.com/life-science/functional-genomics-and-rnai/shrna/): SHCLNV-NM_004566 MISSION® shRNA Lentiviral Transduction Particles for human and SHCLNV-NM_172976 MISSION® shRNA Lentiviral Transduction Particles for mouse.

siRNAs and shRNAs are also commercially available from Santa Cruz biotechnology (sc-44011 and sc-44011-SH, respectively).

In some embodiments this invention is kit, method, composition, pharmaceutical composition, use, PFKFB3 inhibitor, medium of any one of items of this application, wherein PFKFB3 inhibitor is MicroRNA.

MicroRNAs (abbreviated miRNA) are small non-coding RNA molecules (containing about 22 nucleotides) naturally occurring in plants, animals and some viruses, that functions in RNA silencing and post-transcriptional regulation of gene expression.

MicroRNA mimics are double-stranded RNA oligonucleotides designed to mimic the function of endogenous, mature microRNAs. Micro RNA mimics are chemically enhanced with the ON-TARGET modification pattern to preferentially program RISC with the active microRNA strand. Predesigned micro RNAmimics are available for all human, mouse, and rat microRNAs, for example miRIDIAN microRNA Mimic from Dharmacon (http://dharmacon.horizondiscovery.com/rnai/microrna/miridian-microrna-mimic/) and miScript miRNA Mimics from Qiagen (https://www.qiagen.com/us/shop/pcr/real-time-pcr-enzymes-and-kits/miscript-mirna-mimics/)

Non-exclusive list of microRNA that may target PFKFB3: hsa-miR-224-5p, hsa-miR-7110-3p, hsa-miR-3160-5p, hsa-miR-608, hsa-miR-940, hsa-miR-6893-5p, hsa-miR-6808-5p, hsa-miR-6791-3p, hsa-miR-513a-3p, hsa-miR-6829-3p, hsa-miR-3606-3p, hsa-miR-513c-3p, hsa-miR-1468-3p, hsa-miR-4731-5p, hsa-miR-4465, hsa-miR-26a-5p, hsa-miR-4660, hsa-miR-26b-5p, hsa-miR-1297, hsa-miR-6814-5p, hsa-miR-5692a, hsa-miR-4297

CRISPR-CAS9

In some embodiments this invention is kit, method, composition, pharmaceutical composition, use, PFKFB3 inhibitor, medium of any one of items of this application, wherein PFKFB3 inhibitor is is a gene therapy, for example but not limited to therapy comprising CRISPR-CAS9.

PFKFB3 may be inhibited be editing PFKFB3 gene for the purposes of this application such as neuroprotection or anti-aging treatment. Genome editing tools include engineered nucleases, i.e meganucleases, zinc finger nucleases (ZFNs), transcription activator-like effector-based nucleases (TALEN), and the clustered regularly interspaced short palindromic repeats (CRISPR/Cas9) system. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’).

Meganucleases are naturally occurring endonucleases characterized by a large recognition site (double-stranded DNA sequences of 12 to 40 base pairs); as a result this site generally occurs only once in any given genome. Custom meganucleases may be produced by modifying the specificity of existing meganucleases by introducing a small number of variations to the amino acid sequence and then selecting the functional proteins on variations of the natural recognition site. Meganuclease design methods range from high-throughput experimental screening to in silico physical models and machine learning model [Zaslavskiy M, Bertonati C, Duchateau P, Duclert A, Silva G H. Efficient design of meganucleases using a machine learning approach. BMC Bioinformatics. 2014; 15:191].

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence, so when combined with a nuclease, DNA can be cut at specific locations [Boch J (February 2011). “TALEs of genome targeting”. Nature Biotechnology. 29 (2): 135-6].

Zinc finger nucleases (ZFNs) are hybrid proteins composed of a nonspecific cleavage domain from the Type IIS restriction enzyme FokI and a DNA-binding domain made up of zinc fingers (ZFs). The number of fingers in each ZFN can be varied. The minimum number to achieve adequate affinity is three fingers, and combinations up to six have been produced and tested in some contexts. For purposes strictly of genomic cleavage, three-finger and four-finger ZFNs have been used successfully [Carroll D, Morton J J, Beumer K J, Segal D J. Design, construction and in vitro testing of zinc finger nucleases. Nat Protoc. 2006; 1(3):1329-41]

The CRISPR/Cas9 system is widely used for various genome editing approaches in cultured cells and living organisms and was broadly explored for preclinical applications. CRISPR/CRISPR-associated (Cas) systems use Streptococcus pyogenes Cas9 nuclease that is targeted to a genomic site by complexing with a synthetic guide RNA (sgRNA) binds to its complementary target protospacer sequence preceding a protospacer adjunct motif (PAM) recognized by Cas9. CRISPR/Cas9 generates a double-strand break that is usually repaired by non-homologous end-joining (NHEJ), which is error-prone and conducive to frameshift mutations resulting in knock-out alleles of genes

Adeno-associated viral vectors (AAV) are commonly used for in vivo gene delivery due to their low immunogenicity and range of serotypes allowing preferential infection of certain tissues. Since packaging Streptococcus pyogenes (SpCas9) and a chimeric sgRNA together (˜4.2 kb) into an AAV vector is challenging due to the low packaging capacity of AAV (˜4.5 kb.) these elements are packed dual-vector system is used.

AAV CRISPR/CAS9 systems are commercially available, for example from Takara (https://www.takarabio.com/products/gene-function/viral-transduction/adeno-associated-virus-(aav)/vector-systems/crispr/cas9-system).

sgRNA targeting PFKFB3 (chr10):
Exon 7
AATGCGACAGGTGATTCCCGTGG,
Exon 10
TTACCGCTACCCCACCGGGGAGG,
Exon 4
AGCTACCTGGCGAAAGAAGGGGG,
Exon 9
TCGACGCGGTGAGTCCTGGGAGG,
Exon 1
AGGTAGGAGTCCCGGTGACGCGG,
Exon 11
CAGGTACCTCGGGCAGGTCGTGG,
Exon 1
TTTCCTGAAGGGCATCGCGCCGG,
Exon 10
ACCCTGAGGAGTATGCGCTGCGG,
Exon 11
GGCAAGCAGGCAGCGCAGGACGG,
Exon 9
GGCGCTCAATGAGATCGACGCGG.

CRISPR PFKFB3 Knockout Kit is available from Origene (https://www.origene.com/) for mouse (CAT #: KN513141) and human (CAT #: KN206043), GeneCopoeia (http://www.genecopoeia.com/product/search3/?s=PFKFB3&search_type=1 &tags %5B %5D=7824&tags %5B %5D=7833&utm_source=genecards&utm_medium=referral&utm_campaign=product), and Santa Cruz Biotechnology (https://www.scbt.com/scbt/browse/PFK-2-CRISPR-Plasmids/_/N-irgxre).

In some embodiments this invention is a Kit, method, composition, pharmaceutical composition, use, PFKFB3 inhibitor, medium of any one items described below or anywhere in this application, wherein PFKFB3 inhibitor comprises RNAi molecule or gene therapy selected from the described above or its analog.

In some embodiments this invention is a Kit, method, composition, pharmaceutical composition, use, PFKFB3 inhibitor, medium of any one items described below or anywhere in this application, wherein PFKFB3 inhibitor comprises RNAi molecule or gene therapy selected from the described above or its analog for use in rejuvenation or any other anti-aging use selected from this application.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Compounds such as AZ67 and its analogs, including but not limited to those described in the following publication are known in the art and their methods of synthesis are described in J Med Chem. 2015 Apr. 23; 58(8):3611-25. doi: 10.1021/acs.jmedchem.5b00352. Epub 2015 Apr. 13.

Structure-Based Design of Potent and Selective Inhibitors of the Metabolic Kinase PFKFB3. For some known compounds comprised in the disclosed inventions the references for methods of their preparations are available in Table 5.

Tables. Table 1

“Declines”.

Non-limiting list of parameters which age related change is regarded as age related decline and which can be changed into younger state or stabilized or its further change into the older state delayed by anti-aging intervention of this disclosure

| Field | Units |
|---|---|
| Standing height | cm |
| Forced expiratory volume in 1-second (FEV1) | litres |
| Leg fat-free mass (right) | Kg |
| Leg predicted mass (right) | Kg |
| Basal metabolic rate | KJ |
| Forced vital capacity (FVC) | litres |
| Leg fat-free mass (left) | Kg |
| Leg predicted mass (left) | Kg |
| Systolic blood pressure, automated reading | mmHg |
| Heel bone mineral density (BMD) (left) | g/cm2 |

Heel quantitative ultrasound index (QUI), direct entry (left)

| | |
|---|---|
| Whole body fat-free mass | Kg |
| Whole body water mass | Kg |
| Heel bone mineral density (BMD) T-score, | Std. Devs |
| automated (left) | |
| Speed of sound through heel (left) | m/s |
| Sitting height | cm |
| Heel bone mineral density (BMD) (right) | g/cm2 |

Heel quantitative ultrasound index (QUI), direct entry (right)

| | |
|---|---|
| Speed of sound through heel (right) | m/s |
| Heel bone mineral density (BMD) T-score, | Std. Devs |
| automated (right) | |
| Peak expiratory flow (PEF) | litres/min |
| Leg fat percentage (left) | percent |
| Trunk fat-free mass | Kg |
| Leg fat percentage (right) | percent |
| Trunk predicted mass | Kg |
| Hand grip strength (left) | Kg |
| Heel broadband ultrasound attenuation (left) | dB/MHz |
| Heel broadband ultrasound attenuation (right) | dB/MHz |
| Hand grip strength (right) | Kg |
| Duration to first press of snap-button in each round | milliseconds |
| Mean time to correctly identify matches | milliseconds |
| Body fat percentage | percent |
| Trunk fat percentage | percent |
| Body mass index (BMI) | Kg/m2 |
| Leg fat mass (left) | Kg |
| Arm fat-free mass (left) | Kg |
| Arm predicted mass (left) | Kg |
| Arm fat-free mass (right) | Kg |
| Haematocrit percentage | percent |
| Arm predicted mass (right) | Kg |
| Waist circumference | cm |
| Leg fat mass (right) | Kg |
| Haemoglobin concentration | grams/declitre |
| Arm fat percentage (left) | percent |
| Ankle spacing width (left) | mm |
| Whole body fat mass | Kg |
| Body mass index (BMI) | Kg/m2 |
| Pulse wave peak to peak time | milliseconds |
| Arm fat percentage (right) | percent |
| Weight | Kg |
| Mean corpuscular volume | femtolitres |
| Trank fat mass | Kg |

Pulse wave Arterial Stiffness index

| | |
|---|---|
| Ankle spacing width (right) | mm |
| Platelet crit | percent |
| Red blood cell (erythrocyte) count | 10\hat{ }12 cells/Litre |
| Mean sphered cell volume | femtolitres |
| Mean platelet (thrombocyte) volume | femtolitres |
| Weight | Kg |
| Arm fat mass (left) | Kg |
| Lymphocyte percentage | percent |
| Neutrophill percentage | percent |
| Arm fat mass (right) | Kg |
| Impedance of leg (left) | ohms |
| Mean reticulocyte volume | femtolitres |
| Platelet count | 10\hat{ }9 cells/Litre |
| Mean corpuscular haemoglobin | picograms |
| Impedance of leg (right) | ohms |
| Red blood cell (erythrocyte) distribution width | percent |
| Pulse rate, automated reading | bpm |
| Impedance of whole body | ohms |
| Diastolic blood pressure, automated reading | mmHg |
| Lymphocyte count | 10\hat{ }9 cells/Litre |

Number of measurements made

| | |
|---|---|
| Neutrophill count | 10\hat{ }9 cells/Litre |
| Monocyte percentage | percent |
| Hip circumference | cm |
| Monocyte count | 10\hat{ }9 cells/Litre |
| Platelet distribution width | percent |
| Mean corpuscular haemoglobin concentration | grams/decilitre |
| Immature reticulocyte fraction | ratio |
| Impedance of arm (right) | ohms |
| Reticulocyte percentage | percent |

Number of times snap-button pressed

| | |
|---|---|
| White blood cell (leukocyte) count | 10\hat{ }9 cells/Litre |
| Pulse rate | bpm |
| High light scatter reticulocyte count | 10\hat{ }12 cells/Litre |
| Basophill percentage | percent |
| Impedance of arm (left) | ohms |

Pulse wave reflection index

| | |
|---|---|
| Eosinophill count | 10\hat{ }9 cells/Litre |
| Nucleated red blood cell count | 10\hat{ }9 cells/Litre |
| Eosinophill percentage | percent |
| Basophill count | 10\hat{ }9 cells/Litre |
| Reticulocyte count | 10\hat{ }12 cells/Litre |
| High light scatter reticulocyte percentage | percent |
| Nucleated red blood cell percentage | percent |

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

As used in the specification and appended claims, unless specified to the contrary, the following terms have the meaning indicated below.

“Amino” refers to the —NH₂ radical, optionally substituted with one or more groups

selected from, for example: alkyl, fluoroalkyl, cycloalkyl, cycloalkylalkyl, aryl, aralkyl, heterocycloalkyl, heterocycloalkylalkyl, heteroaryl and heteroarylalkyl.

“Cyano” or “nitrile” refers to the —CN radical.

“Hydroxy” or “hydroxyl” refers to the —OH radical.

“Nitro” refers to the —NO₂ radical.

“Oxo” refers to the ═O substituent.

“Thioxo” refers to the ═S substituent.

“Oximo” or “hydroximino” refer to the ═N—OH substituent

“Alkyl” refers to a linear or branched hydrocarbon chain radical, which is fully saturated. Alkyl may have from one to thirty carbon atoms. Alkyl may be attached to the rest of the molecule by a single bond.

An alkyl comprising up to 30 carbon atoms is referred to as a C₁-C₃₀ alkyl, likewise, for example, an alkyl comprising up to 12 carbon atoms is a C₁-C₁₂ alkyl. An alkyl comprising up to 6 carbons is a C₁-C₆ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₈ alkyl, C₃-C₈ alkyl, C₄-C₈ alkyl, and C₅-C₁₂ alkyl. Representative alkyl groups include, but are not limited to, methyl, ethyl, n-propyl, 1-methylethyl (isopropyl), n-butyl, i-butyl, s-butyl, n-pentyl, 1,1-dimethylethyl (t-butyl), 2-ethylpropyl, and the like. Representative linear alkyl groups include, but are not limited to, methyl, ethyl, n-propyl, n-butyl, n-pentyl and the like. In certain embodiments, an alkyl group is optionally substituted by one or more of the following substituents: halo, cyano, nitro, oxo, thioxo, imino, oximo, trimethylsilyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkenyl” refers to a straight or branched hydrocarbon chain radical, containing at least one carbon-carbon double bond. In certain embodiments, alkenyl comprises from two to twelve carbon atoms. In certain embodiments, an alkenyl comprises two to eight carbon atoms. In certain embodiments, an alkenyl comprises two to six carbon atoms. In other embodiments, an alkenyl comprises two to four carbon atoms. The alkenyl may be attached to the rest of the molecule by a single bond, for example, ethenyl (i.e., vinyl), prop-1-enyl (i.e., allyl), but-1-enyl, pent-1-enyl, penta-1,4-dienyl, and the like. Alkenyl may be attached to the rest of the molecule by a double bond, e.g., ═CH₂, ═CH(CH₂)₃CH₃. In certain embodiments, an alkenyl group is optionally substituted by one or more of the following substituents: halo, cyano, nitro, oxo, thioxo, imino, oximo, trimethylsilyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkynyl” refers to a straight or branched hydrocarbon chain radical group, containing at least one carbon-carbon triple bond. In certain embodiments, alkynyl comprises from two to twelve carbon atoms. In certain embodiments, an alkynyl comprises two to eight carbon atoms. In certain embodiments, an alkynyl comprises two to six carbon atoms. In other embodiments, an alkynyl has two to four carbon atoms. The alkynyl may be attached to the rest of the molecule by a single bond, for example, ethynyl, propynyl, butynyl, pentynyl, hexynyl, and the like. In certain embodiments, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having, for example, from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In certain embodiments, an alkylene comprises five to eight carbon atoms (e.g., C₅-C₈ alkylene). In certain embodiments, an alkylene comprises two to five carbon atoms (e.g., C₂-C₅ alkylene). In certain embodiments, an alkylene comprises three to five carbon atoms (e.g., C₃-C₅ alkylene). In certain embodiments, an alkylene chain is optionally substituted by one or more of the following substituents: halo, cyano, nitro, oxo, thioxo, imino, oximo, trimethylsilyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain may be attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond.

The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene).

In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Aminoalkyl” refers to a radical of the formula —R_{c}—N(R^{a})₂ or —R_{c}—N(R^{a})—R_{c}, where each R_{c} is independently an alkylene chain as defined above, for example, methylene, ethylene, and the like; and each R^{a} is independently hydrogen, or a substituent, e.g., alkyl, fluoroalkyl, cycloalkyl, cycloalkylalkyl, aryl, aralkyl, heterocycloalkyl, heterocycloalkylalkyl, heteroaryl or heteroarylalkyl.

“Alkoxy” refers to a radical of the formula —O-alkyl where alkyl is as defined herein. Unless stated otherwise specifically in the specification, an alkoxy group may be optionally substituted as described above for alkyl.

“Aryl” refers to a radical derived from an aromatic monocyclic hydrocarbon or aromatic multicyclic hydrocarbon ring system by removing a hydrogen atom from a ring carbon atom. Aryl may includes cycles with five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The term “arylene” refers to the diradical derived from aryl as defined herein and is exemplified by phenylene and the like. In certain embodiments, an aryl or arylene is optionally substituted by one or more of the following substituents: alkyl, alkenyl, alkynyl, halo, fluoroalkyl, cyano, nitro, aryl, aralkyl, aralkenyl, aralkynyl, cycloalkyl, cycloalkylalkyl, heterocycloalkyl, heterocycloalkylalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocycloalkyl (optionally substituted with one or more alkyl groups), heterocycloalkylalkyl, heteroaryl or heteroarylalkyl, or two R^{a} attached to the same nitrogen atom are combined to form a heterocycloalkyl, each R^{b} is independently a direct bond or a straight or branched alkylene or alkenylene chain, and R_{c} is a straight or branched alkylene or alkenylene chain, and where each of the above substituents is unsubstituted unless otherwise indicated.

“Aralkyl” refers to a radical of the formula —R_{c}-aryl where R_{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

The term “Cₓ-C_{y}” when used in conjunction with a chemical moiety, such as alkyl, alkenyl, or alkynyl is meant to include groups that contain from x to y carbons in the chain. For example, the term “Cₓ-C_{y} alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. The terms “Cₓ-C_{y} alkenyl“and” Cₓ-C_{y} alkynyl” refer to substituted or unsubstituted unsaturated aliphatic groups analogous in length and possible substitution to the alkyls described above, but that contain at least one double or triple bond respectively.

“Cycloalkyl” refers to a saturated or partially unsaturated, monocyclic or polycyclic hydrocarbon radical. In certain embodiments, the cycloalkyl includes fused (when fused with an aryl or a heteroaryl ring, the cycloalkyl is bonded through a non-aromatic ring atom) or bridged ring systems. In certain embodiments, the cycloalkyl comprises from three to twenty carbon atoms. In certain embodiments, a cycloalkyl comprises three to ten carbon atoms. In other embodiments, a cycloalkyl comprises five to seven carbon atoms. The cycloalkyl may be attached to the rest of the molecule by a single bond. In some embodiments, the cycloalkyl is fully saturated. Examples of monocyclic fully saturated cycloalkyls include, e.g., cyclopropyl, cyclobutyl, cyclopentyl, cyclohexyl, cycloheptyl, and cyclooctyl. Polycyclic fully saturated cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. In some embodiments, the cycloalkyl is partially unsaturated or also known as a “cycloalkenyl”.

Examples of cycloalkenyls include cyclopentenyl, cyclohexenyl, cycloheptenyl, cyclooctenyl, and the like. In certain embodiments, the cycloalkyl is optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

The term “cycloalkylene” refers to the diradical derived from cycloalkyl as defined herein. In some embodiments the cycloalkylene is fully saturated and is exemplified by cyclopropylene, cyclobutylene, cyclopentylene, cyclohexylene, and the like. In some embodiments, the cycloalkylene is partially unsaturated or also known as a “cycloalkenylene” and is exemplified by 1,2-cyclobut-1-enylene, 1,4-cyclohex-2-enylene and the like.

“Cycloalkylalkyl” refers to a radical of the formula —R^{c}-cycloalkyl where R^{c} is an alkylene chain as defined above. The alkylene chain and the cycloalkyl radical are optionally substituted as described above.

Halo” or “halogen” refers to a halogen radical, e.g., bromo, chloro, fluoro or iodo. In some embodiments, halogen refers to chloro or fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In certain embodiments, a haloalkyl group may be optionally substituted.

“Heterocycloalkyl” refers to a saturated or partially unsaturated ring radical that comprises two to twenty carbon atoms and at least one heteroatom. In certain embodiments, the heteroatoms are independently selected from N, O, Si, P, B, and S atoms. The heterocycloalkyl may be selected from monocyclic or bicyclic, (fused when fused with an aryl or a heteroaryl ring, the heterocycloalkyl is bonded through a non-aromatic ring atom) or bridged ring systems. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. In certain embodiments, the heterocycloalkyl comprises from five to twenty carbon atoms. In certain embodiments, a heterocycloalkyl comprises five to ten carbon atoms. In other embodiments, a heterocycloalkyl comprises five to seven carbon atoms. In some embodiments, the heterocycloalkyl is fully saturated. Examples of fully saturated heterocycloalkyl radicals include, but are not limited to, 1,4-dioxanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocycloalkyl is partially unsaturated or also known as a “heterocycloalkenyl”. Examples of heterocycloalkenyl include 1,2,3,4-tetrahydropyridinyl, 1,2-dihydropyridinyl, 2-oxo-1,3-dioxolyl and the like. In certain embodiments, the heterocycloalkyl is optionally substituted by one or more of the following substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

The term “heterocycloalkylene” refers to the diradical derived from heterocycloalkyl as defined herein. In some embodiments the heterocycloalkylene is fully saturated and is exemplified by azetidinyllene, aziridinylene, pyrrolidylene, piperidinylene, morpholinylene, and the like. In some embodiments, the heterocycloalkylene is partially unsaturated or also known as a “heterocycloalkenylene”.

“Heterocycloalkylalkyl” refers to a radical of the formula —R_{c}-heterocycloalkyl where R_{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylslkyl radical is optionally substituted as defined above for an alkylene chain. The heterocycloalkyl part of the heterocycloalkylalkyl radical is optionally substituted as defined above for a heterocycloalkyl group.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. In some embodiments, the heteroaryl is a 5-membered heteroaryl. In some embodiments, the heteroaryl is a 6-membered heteroaryl. For purposes of this invention, the heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). The term “heteroarylene” refers to the diradical derived from heteroaryl as defined herein and is exemplified by pyridinylene, pyrimidinylene, pyrazinylene, and the like. Unless stated otherwise specifically in the specification, a heteroaryl group is optionally substituted by one or more of the following substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R_{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R_{c} is a straight or branched alkylene or alkenylene chain, and where each of the above substituents is unsubstituted unless otherwise indicated.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

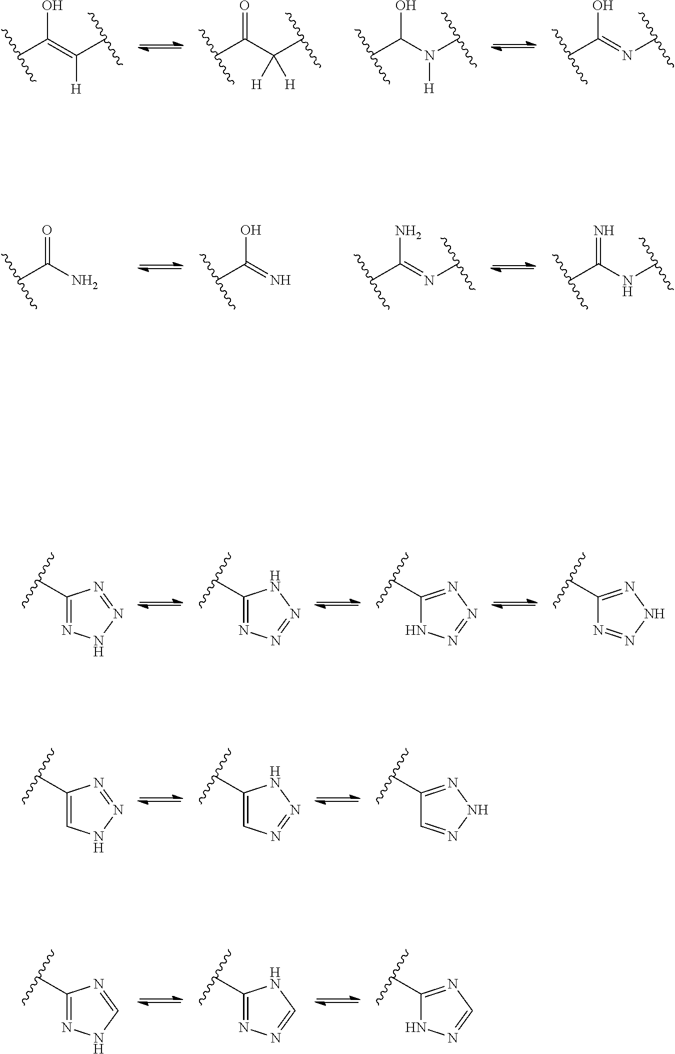

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution. “Optionally substituted” and “substituted or unsubstituted” and “unsubstituted or substituted” are used interchangeably herein.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, nicotinic acid, succinic acid, fumaric acid, formic acid, tartaric acid, camphor-10-sulfonic acid, citric acid, benzoic acid, cinnamic acid, isonipecotinic acid, levulinic acid, maleic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, isonipecotinates, levuliates, oxalates, malonates, nicotinates, succinate, suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are as a rule prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared by addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, triethanolamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

In a specific embodiment and in this context, the term “pharmaceutically acceptable carrier” can mean a carrier, excipient or diluent approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a specific carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E.W. Martin.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range.

The term “comprising” (and related terms such as “comprise” or “comprises” or “having” or “including”) is not intended to exclude that which in other certain embodiments, for example, an embodiment of any composition of matter, composition, method, or process, or the like, described herein, “consist of” or “consist essentially of” the described features.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The term “modulate,” as used herein, means to interact with a target protein either directly or indirectly so as to alter the activity of the target protein, including, by way of example only, to inhibit the activity of the target, or to limit or reduce the activity of the target, or to increase certain activity of a target compared to the magnitude of the activity in the absence of the modulator

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has been made.

As used herein, “PFK1” refers to phosphofructokinase 1, also known as 6-phosphofructo-1-kinase.

As used herein, “PFK2” refers to phosphofructokinase 2, also known as 6-phosphofructo-2-kinase/fructose-2,6-bisphosphatase.

As used herein, “PFKFB1,” “PFKFB2,” “PFKFB3,” “PFKFB4” refer to specific forms of PFK2.

The term “subject,” as used herein, generally refers to an animal, such as a mammalian species (e.g., mouse or human) or avian (i.e., bird) species, nematode (e.g., C. elegans), or other organism, such as a plant. More specifically, the subject can be a vertebrate, e.g., a mammal such as a mouse, a primate, a simian or a human. Preferably, the subject is a human. Preferably, the subject is more than 40 years old. Animals include, but are not limited to, farm animals, sport animals, and pets. A subject can be a healthy individual, an individual that has or is suspected of having a disease or a predisposition to the disease, or an individual that is in need of therapy or suspected of needing therapy, or an aged or frail individual. A subject can be any human being.

In some embodiments, by treating or preventing an age-related disease or disorder, any anti-aging treatment is meant. Anti-aging treatment includes (but is not limited to) treatments leading to prevention, amelioration or lessening the effects of aging, decreasing or delaying an increase in the biological age, slowing rate of aging; treatment, prevention, amelioration and lessening the effects of frailty or at least one of aging related diseases and conditions or declines or slowing down the progression of such decline (including but not limited to those indicated in Table 1, “Declines”), condition or disease, increasing health span or lifespan, rejuvenation, increasing stress resistance or resilience, increasing rate or other enhancement of recovery after surgery, radiotherapy, disease and/or any other stress, prevention and/or the treatment of menopausal syndrome, restoring reproductive function, eliminating or decrease in spreading of senescent cells, decreasing all-causes or multiple causes of mortality risks or mortality risks related to at least one or at least two of age related diseases or conditions or delaying in increase of such risks, decreasing morbidity risks. The treatment leading to the modulating at least one of biomarkers of aging into more youthful state or slowing down its change into “elder” state is also regarded to be an anti-aging treatment, including but not limited to biomarkers of aging which are visible signs of aging, such as wrinkles, grey hairs etc. In some embodiments, an age-related disease or disorder is selected from: atherosclerosis, cardiovascular disease, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence (including but not limited to age related decline in immune response to vaccines, age related decline in response to immunotherapy etc.), myocardial infarction, acute coronary syndrome, sarcopenia, sarcopenic obesity, senile osteoporosis, urinary incontinence etc. Cancer survivors, patients under chemiotherapy and radiotherapy and others comparable stress and as well as HIV patients may suffer accelarated aging, treatment against such accelerated aging or its consequences is also regarded as anti-aging treatment, as well as preventive measures against it.

Aging-related changes in any parameter or physiological metric are also regarded as age-related conditions, including but not limited to aging related change in blood parameters, heart rate, cognitive functions/decline, bone density, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), and time for recovery after any stress (wound, operation, chemotherapy, disease, change in lifestyle etc.). In some embodiments, the age-related disorder is a cardiovascular disease. In some embodiments, the age-related disorder is a bone loss disorder. In some embodiments, the age-related disorder is a neuromuscular disorder. In some embodiments, the age-related disorder is a neurodegenerative disorder or a cognitive disorder. In some embodiments, the age-related disorder is a metabolic disorder. In some embodiments, the age-related disorder is sarcopenia, osteoarthritis, chronic fatigue syndrome, senile dementia, mild cognitive impairment due to aging, schizophrenia, Huntington's disease, Pick's disease, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, Lewy body disease, amyotrophic lateral sclerosis (ALS), mild cognitive impairment, pre-dementia, dementia, progressive subcortical gliosis, progressive supranuclear palsy, thalamic degeneration syndrome, hereditary aphasia, myoclonus epilepsy, macular degeneration, or cataracts. Aging related change in any parameter of organism is also regarded as an aging related condition, including but not limited to aging related change in at least one of the parameter selected from the Table “Declines”. In some embodiments, term “anti-aging treatment” means treatment of disease or condition mediated by PFKFB3, excluding cancer. In some embodiments, term “anti-aging treatment” means treatment increasing resistance to radiation. In some embodiments, term “anti-aging treatment” means treatment of disease or age related condition or neurodegeneration associated with the alleviated level of PFKFB3 in subject's cells. some embodiments of this invention “aged subject” is understood as a human being of chronological age (or in some embodiments, of biological age) of 30 years or older, 35 years or older, 40 years or older, 45 years or older, 50 years or older, 55 years or older, 60 years or older, 65 years or older, 70 years or older, 75 years or older, 80 years or older, 85 years or older, 90 years or older, 95 years or older. In some embodiments of this invention “aged subject” is understood as a frail human (or other animal).

In some embodiments, an age related disease or disorder is selected from: atherosclerosis, cardiovascular disease, arthritis, cataracts, osteoporosis, type 2 diabetes, hypertension, neurodegenaration, including but not limited to Alzheimer's disease, dementia, Parkinson's disease), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, myocardial infarction, acute coronary syndrome, sarcopenia, sarcopenic obesity, senile osteoporosis, urinary incontinence or diseases and conditions mentioned in “Frameworks for Proof-of-Concept Clinical Trials of Interventions That Target Fundamental Aging Processes”. Justice et al., 2016), Juvenescence: Investing in the Age of Longevity, Mellon at al., 2017) etc.

In some embodiments, age related, aging-related or ageing-related means “caused by pathological processes which persistently lead to the loss of organism's adaptation and progress in older ages”.

“Indirect Target” means effector upstream or downstream of a PFKFB3, which can be an element (protein, small molecule, cell, electrolytes, antibodies, antigens, hormones, microRNA, RNA etc.) which is upstream or downstream in a pathway in relation to PFKFB3. In some embodiments Indirect Target means any upstream or downstream element, which modulation or reduction effects or mimics the effect of PFKFB3 reduction, inhibition or degradation, optionally that have anti-aging or neuroprotective effect.

In some embodiments, everything related to PFKFB3 relates to Indirect Target, e.g. when it is said that PFKFB3 inhibitor is for use as neuroprotector, in such embodiment it will mean that the modulator of Indirect Target is for use as neuroprotector.

In some embodiments, term “Small molecule” means an individual compound with molecular weight less than about 2000 daltons in size, usually less than about 1500 daltons in size, more usually less than about 750 daltons in size, preferably less than about 500 daltons in size, although molecules larger than 2000 daltons in size will also be PFKFB3 inhibitors herein.

The terms “pharmaceutical composition” and formulation are used interchangeably herein.

The term “selected from . . . ” means “one or more of” (e.g. bind one or more of the following proteins . . . ”).

In this context, the term “PFKFB3 inhibitor(s) selectively bind(s) a PFKFB3” can mean the following:

The term “solely” means that the PFKFB3 inhibitor(s) bind(s) exclusively to a PFKFB3, i.e. the PFKFB3 inhibitor(s) do(does) not bind to proteins other than a PFKFB3.

Compounds

The compounds, and compositions comprising the compounds described herein, are useful for the treatment of diseases or conditions where the modulation of PFKFB3 and/or PFKFB4 has beneficial effect. In certain embodiments, compounds, and compositions comprising the compounds, described herein are useful for the treatment of diseases or conditions wherein the inhibition of kinase activity of PFKFB3 and/or PFKFB4 has beneficial effect or for manufacturing of corresponding medications.

The compounds, and compositions comprising the compounds described herein, are useful for many uses, including but not limited to the treatment of cancer, neudegenerative and aging related diseases or conditions where the modulation of PFKFB3 has beneficial effect. In certain embodiments, compounds, and compositions comprising the compounds, described herein are useful for the treatment of diseases or conditions wherein the inhibition of kinase activity of PFKFB3 has beneficial effect or for manufacturing of corresponding medications.

In some embodiments, the PFKFB3 inhibitor selectively binds the PFKFB3 with a KD of less than 1×10-3M. In some embodiments, the PFKFB3 inhibitor selectively binds the PFKFB3 with a KD of less than 1×10-4 M. In some embodiments, the PFKFB3 inhibitor selectively binds the PFKFB3 with a KD of less than 1×10-5 M. In some embodiments, the PFKFB3 inhibitor selectively binds the PFKFB3 with a KD of less than 1×10-6 M. In some embodiments, the PFKFB3 inhibitor selectively binds the PFKFB3 with a KD of less than 1×10-7 M. In some embodiments, the PFKFB3 inhibitor selectively binds the PFKFB3 with a KD of less than 1×10-8 M. In some embodiments, the PFKFB3 inhibitor selectively binds the PFKFB3 with a KD of less than 1×10-9 M. In some embodiments, the PFKFB3 inhibitor selectively binds the PFKFB3 with a KD of less than 1×10-10 M. In some embodiments, the PFKFB3 inhibitor selectively binds the PFKFB3 with a KD of less than 1×10-12 M. In some embodiments, the PFKFB3 inhibitor selectively binds the PFKFB3 with a KD of less than 1×10-15 M. In certain embodiments, the PFKFB3 inhibitor selectively binds the PFKFB3 with a KD of about 1×10-3M to about 1×10-15M. In certain embodiments, the PFKFB3 inhibitor selectively binds the PFKFB3 with a KD of about 1×10-3M to about 1×10-14M. In certain embodiments, the PFKFB3 inhibitor selectively binds the PFKFB3 with a KD of about 1×10-3M to about 1×10-13M. In certain embodiments, the PFKFB3 inhibitor selectively binds the PFKFB3 with a KD of about 1×10-3M to about 1×10-12M. In certain embodiments, the PFKFB3 inhibitor selectively binds the PFKFB3 with a KD of about 1×10-3M to about 1×10-11 M. In certain embodiments, the PFKFB3 inhibitor selectively binds the PFKFB3 with a KD of about 1×10-3M to about 1×10-10M. In certain embodiments, the PFKFB3 inhibitor selectively binds the PFKFB3 with a KD of about 1×10-3M to about 1×10-9M. In certain embodiments, the PFKFB3 inhibitor selectively binds the PFKFB3 with a KD of about 1×10-3M to about 1×10-8M. In certain embodiments, the PFKFB3 inhibitor selectively binds the PFKFB3 with a KD of about 1×10-3M to about 1×10-7M. In certain embodiments, the PFKFB3 inhibitor selectively binds the PFKFB3 with a KD of about 1×10-3M to about 1×10-6M. In certain embodiments, the PFKFB3 inhibitor selectively binds the PFKFB3 with a KD of about 1×10-6M to about 1×10-15M. In certain embodiments, the PFKFB3 inhibitor selectively binds the PFKFB3 with a KD of about 1×10-9M to about 1×10-15M. In certain embodiments, the PFKFB3 inhibitor selectively binds the PFKFB3 with a KD of about 1×10-12M to about 1×10-15M. In certain embodiments, the PFKFB3 inhibitor selectively binds the PFKFB3 with a KD of about 1×10-6M to about 1×10-12M. In certain embodiments, the PFKFB3 inhibitor selectively binds the PFKFB3 with a KD of about 1×10-6M to about 1×10-9M.

Disclosed herein is a compound of Formula (I):

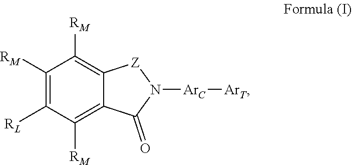

or a pharmaceutically acceptable salt thereof, wherein:

Z is selected from —C(═O)— and —C(R^{a})(R^{b})—;

R^{a} and R^{b} are independently selected from hydrogen, hydroxyl, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

Ar_{C} is selected from C₃-C₈ cycloalkenylene, C₂-C₈ heterocycloalkenylene, arylene, and heteroarylene; wherein Ar_{C} is substituted with one or more R_{C};

each R_{C} are independently selected from —CN, —OH, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, optionally substituted —O—C₂-C₈ heterocycloalkyl, optionally substituted heteroaryl, optionally substituted aryl, —C(═O)OH, —C(═O)OR³, —C(═O)NR⁴R⁵, —S(═O)₂NR⁴R⁵, —NHC(═O)H, —NHC(═O)R⁶, —NHS(═O)₂R⁶, and —C(═O)NHS(═O)₂R⁶;

each R³ is independently optionally substituted C₁-C₆ alkyl or optionally substituted C₃-C₈ cycloalkyl;

each R⁴ and R⁵ is independently selected from hydrogen, optionally substituted C₁-C₆ alkyl, optionally substituted C₃-C₈ cycloalkyl, optionally substituted aryl, and optionally substituted heteroaryl;

or R⁴ and R⁵ are taken together with the N to which they are attached to form an optionally substituted C₂-C₈ heterocycloalkyl;

each R⁶ are independently selected from optionally substituted C₁-C₆ alkyl, optionally substituted C₃-C₈ cycloalkyl, optionally substituted aryl, and optionally substituted heteroaryl;

Ar_{T} is selected from pyridinyl, pyrimidinyl, pyridazinyl, pyrazinyl, phenyl, pyrrolyl, furanyl, thiophenyl, pyrazolyl, imidazolyl, triazolyl, and tetrazolyl, wherein Ar_{T} is optionally substituted;

each R_{M} is independently selected from hydrogen, halogen, —OH, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

R_{L} is selected from —OH, —CN, optionally substituted C₁-C₆ hydroxyalkyl, optionally substituted C₁-C₆ alkoxy, optionally substituted heteroaryl, —C(═O)OH, —C(═O)OR⁹, —C(═O)NR¹⁰R¹¹, —S(═O)₂NR¹⁰R¹¹, —NHC(═O)H, —NHC(═O)R¹², —NHS(═O)₂R¹² and —C(═O)NHS(═O)₂R¹²;

R⁹ is optionally substituted C₁-C₆ alkyl;

or R⁹ is optionally substituted C₃-C₈ cycloalkyl;

each R¹⁰ and R¹¹ is independently selected from hydrogen, optionally substituted C₁-C₆ alkyl, optionally substituted C₃-C₈ cycloalkyl, optionally substituted aryl, and optionally substituted heteroaryl;

or R¹⁰ and R¹¹ are taken together with the N to which they are attached to form an optionally substituted C₂-C₈ heterocycloalkyl;

R¹² is selected from optionally substituted C₁-C₆ alkyl, optionally substituted C₃-C₈ cycloalkyl, optionally substituted aryl, and optionally substituted heteroaryl;

provided that:

(a) at least one of R_{C} is not —NHCOR⁶ when R_{L} is —NHCOR¹² and Ar_{C} is heterocycloalkenylene or heteroarylene; or

(b) at least one of R_{C} is not -Me when R_{L} is —OMe; or

(c) at least one of R_{C} is not —OEt when R_{L} is —C(═O)OH; or

(d) at least one of R_{C} is not —OH when R_{L} is —C(═O)OH; or

(e) at least one of R_{C} is not -Me when R_{L} is —C(═O)OH; or

(f) at least one of R_{C} is not -Et when R_{L} is —OMe; or

(g) at least one of R_{C} is not optionally substituted benzoxazolyl when R_{L} is —C(═O)OH; or

(h) at least one of R_{C} is not optionally substituted isoindoline-1,3-dione when R_{L} is —C(═O)OH.

In some embodiments of a compound of Formula (I):

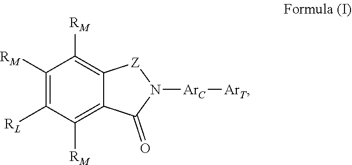

or a pharmaceutically acceptable salt thereof, wherein:

Z is selected from —C(═O)— and —C(R^{a})(R^{b})—;

R^{a} and R^{b} are independently selected from hydrogen, hydroxyl, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR¹R², —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

Ar_{C} is selected from C₃-C₈ cycloalkenylene, C₂-C₈ heterocycloalkenylene, arylene, and heteroarylene; wherein Ar_{C} is substituted with one or more R_{C};

each R_{C} are independently selected from —CN, —OH, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, optionally substituted —O—C₂-C₈ heterocycloalkyl, optionally substituted heteroaryl, optionally substituted aryl, —C(═O)OH, —C(═O)OR³, —C(═O)NR⁴R⁵, —S(═O)₂NR⁴R⁵, —NHC(═O)H, —NHC(═O)R⁶, —NHS(═O)₂R⁶, and —C(═O)NHS(═O)₂R⁶;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, —O—C₂-C₈ heterocycloalkyl, aryl, and heteroaryl are optionally substituted with one or more substituents independently selected from —OH, halogen, —C(═O)OR⁷, —C(═O)R⁶, —C(═O)NR¹R², C₁-C₆alkyl, C₁-C₈ alkoxy, C₃-C₈ cycloalkyl, —O—C₃-C₈cycloalkyl, C₂-C₈ heterocycloalkyl, —O—C₂-C₈ heterocycloalkyl, optionally substituted aryl, optionally substituted heteroaryl, and —NR⁷R⁸;

each R¹ and R² is independently selected from hydrogen, optionally substituted C₁-C₆ alkyl, and optionally substituted C₃-C₈ cycloalkyl;

wherein the C₁-C₆ alkyl is optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl is optionally substituted with one or more substituents independently selected from halogen, —C(═O)OR⁷, —C(═O)NR⁷R⁸, —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R³ is independently C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or each R³ is independently C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁴ and R⁵ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R⁶ are independently selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁷ and R⁸ is independently selected from hydrogen and C₁-C₆ alkyl;

or R⁷ and R⁸ are taken together with the N to which they are attached to form a C₂-C₈ heterocycloalkyl optionally substituted with one or more C₁-C₆alkyl substituents;

Ar_{T} is selected from pyridinyl, pyrimidinyl, pyridazinyl, pyrazinyl, phenyl, pyrrolyl, furanyl, thiophenyl, pyrazolyl, imidazolyl, triazolyl, and tetrazolyl, wherein Ar_{T} is optionally substituted by one or more substituents independently selected from halogen, —OH, —NR⁷R⁸, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;

each R_{M} is independently selected from hydrogen, halogen, —OH, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from —OH, —CN, optionally substituted C₁-C₆ hydroxyalkyl, optionally substituted C₁-C₆ alkoxy, optionally substituted heteroaryl, —C(═O)OH, —C(═O)OR⁹, —C(═O)NR¹⁰R¹¹, —S(═O)₂NR¹⁰R¹¹, —NHC(═O)H, —NHC(═O)R¹², —NHS(═O)₂R¹² and —C(═O)NHS(═O)₂R¹²;

wherein the C₁-C₆ hydroxyalkyl and C₁-C₆alkoxy are optionally substituted with one or more substituents independently selected from halogen, —C(═O)OR⁷, —C(═O)NR¹R², —OH, aryl, heteroaryl, C₁-C₆alkoxy, C₃-C₈ cycloalkyl, —O—C₃-C₈ cycloalkyl, C₂-C₈ heterocycloalkyl, and —O—C₂-C₈ heterocycloalkyl; and

wherein the heteroaryl is optionally substituted with one or more of —OH, —O—C(═O)C₁-C₆ alkyl, (C₁-C₄ alkylene)-O—C(═O)C₁-C₆ alkyl, C₁-C₆ alkyl-(aryl), C₁-C₆ alkyl-(heteroaryl), halogen, —C(═O)OR⁷, —C(═O)R¹², —C(═O)NR¹R², C₁-C₆ alkyl, C₁-C₆ hydroxyalkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR¹R²;

R⁹ is C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁹ is C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R¹⁰ and R¹¹ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl (optionally substituted with —OH, halogen, —C(═O)OR⁷, —C(═O)NR⁷R⁸, C₁-C₆ alkyl, C₁-C₆ alkoxy, —NR⁷R⁸), and heteroaryl (optionally substituted with —OH, halogen, —C(═O)OR⁷, —C(═O)NR⁷R⁸, C₁-C₆ alkyl, C₁-C₆ alkoxy, —NR⁷R⁸, C₃-C₈ cycloalkyl, —O—C₃-C₈ cycloalkyl, C₂-C₈ heterocycloalkyl, or —O—C₂-C₈ heterocycloalkyl); and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R¹⁰ and R¹¹ are taken together with the N to which they are attached to form a C₂-C₈ heterocycloalkyl optionally substituted with one or more substituents independently selected from halogen, —OH, C₁-C₆ alkyl, and C₁-C₆ alkoxy;

R¹² is selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

provided that:

(a) at least one of R_{C} is not —NHCOR⁶ when R_{L} is —NHCOR¹² and Ar_{C} is heterocycloalkenylene or heteroarylene; or

(b) at least one of R_{C} is not -Me when R_{L} is —OMe; or

(c) at least one of R_{C} is not —OEt when R_{L} is —C(═O)OH; or

(d) at least one of R_{C} is not —OH when R_{L} is —C(═O)OH; or

(e) at least one of R_{C} is not -Me when R_{L} is —C(═O)OH; or

(f) at least one of R_{C} is not -Et when R_{L} is —OMe; or

(g) at least one of R_{C} is not optionally substituted benzoxazolyl when R_{L} is —C(═O)OH; or

(h) at least one of R_{C} is not optionally substituted isoindoline-1,3-dione when R_{L} is —C(═O)OH.

In some embodiments of a compound of Formula (I), Z is —C(═O)—. In some embodiments of a compound of Formula (I), Z is —C(R^{a})(R^{b})—, and R^{a} and R^{b} are each independently selected from hydrogen, halogen, —OH, C₁₋₆ alkyl, and C₁₋₆ alkoxy. In some embodiments of a compound of Formula (I), Z is —C(R^{a})(R^{b})—, and R^{a} and R^{b} are each independently selected from hydrogen, fluorine, and methyl. In some embodiments of a compound of Formula (I), Z is —CH₂—.

In some embodiments of a compound of Formula (I), Ar_{C} is arylene or heteroarylene; each substituted with one or more R_{C}. In some embodiments of a compound of Formula (I), Ar_{C} is arylene substituted with one or two R_{C}. In some embodiments of a compound of Formula (I), Ar_{C} is a phenylene substituted with one or two R_{C}. In some embodiments of a compound of Formula (I), Ar_{C} is arylene substituted with one R_{C}. In some embodiments of a compound of Formula (I), Ar_{C} is phenylene substituted with one R_{C}.

In some embodiments of a compound of Formula (I), Ar_{C} is heteroarylene selected from pyridinylene, pyrimidylene, pyrazinylene, and thiophenylene. In some embodiments of a compound of Formula (I), Ar_{C} is heteroarylene substituted with one or two R_{C}. In some embodiments of a compound of Formula (I), Ar_{C} is a monocyclic heteroarylene substituted with one or two R_{C}. In some embodiments of a compound of Formula (I), Ar_{C} is heteroarylene substituted with one R_{C}. In some embodiments of a compound of Formula (I), Ar_{C} is thiophenylene substituted with one R_{C}. In some embodiments of a compound of Formula (I), Ar_{C} is thiophenylene substituted with two R_{C}.

In some embodiments of a compound of Formula (I), each R_{C} are independently selected from —CN, —OH, halogen, optionally substituted C₁-C₆ alkyl, optionally substituted C₃-C₈ cycloalkyl, optionally substituted C₁-C₆ alkoxy, optionally substituted heteroaryl, optionally substituted aryl, —C(═O)OH, —C(═O)OR³, and —C(═O)NR⁴R⁵;

wherein the C₁-C₆ alkyl and C₁-C₆ alkoxy are optionally substituted with one or more substituent independently selected from halogen, —C(═O)OR⁷, —C(═O)NR¹R², —OH, aryl, heteroaryl, C₁-C₆ alkoxy, C₃-C₈ cycloalkyl, and C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆alkyl, C₁-C₆alkoxy, and —NR⁷R⁸.

In some embodiments of a compound of Formula (I), each R_{C} are independently selected from —CN, —OH, halogen, C₁-C₆ alkyl, C₁-C₆ hydroxyalkyl, C₁-C₆ hydroxycycloalkyl, C₁-C₆ alkoxy, heteroaryl, aryl, —C(═O)OH, —C(═O)OR³, and —C(═O)NR⁴R⁵. In some embodiments of a compound of Formula (I), one R_{C} is selected from —CN, —OH, halogen, C₁-C₆ alkyl, C₁-C₆ hydroxyalkyl, C₁-C₆ hydroxycycloalkyl, C₁-C₆ alkoxy, heteroaryl, aryl, —C(═O)OH, —C(═O)OR³, and —C(═O)NR⁴R⁵; and a second R_{C} is selected from —OH, halogen, C₁-C₆ alkyl, C₁-C₆ alkoxy, or aryl. In some embodiments of a compound of Formula (I), each R_{C} are independently selected from —CN, —C(═O)OH, —C(═O)OR³, and tetrazolyl. In some embodiments of a compound of Formula (I), one R_{C} is selected from —CN, —C(═O)OH, —C(═O)OR³, and tetrazolyl; and a second R_{C} is selected from —OH, halogen, C₁-C₆ alkyl, C₁-C₆ alkoxy, or aryl. In some embodiments of a compound of Formula (I), at least one R_{C} is not ethoxy. In some embodiments of a compound of Formula (I), at least one R_{C} is not ethyl. In some embodiments of a compound of Formula (I), at least one R_{C} is not —OH. In some embodiments of a compound of Formula (I), at least one R_{C} is not methyl. In some embodiments of a compound of Formula (I), at least one R_{C} is not benzoxazolyl. In some embodiments of a compound of Formula (I), at least one R_{C} is not isoindoline-1,3-dione. In some embodiments of a compound of Formula (I), at least one of R_{C} is —CN. In some embodiments of a compound of Formula (I), at least one of R_{C} is —C(═O)OH. In some embodiments of a compound of Formula (I), at least one of R_{C} is tetrazolyl. In some embodiments of a compound of Formula (I), at least one of R_{C} is —C(═O)OR³. In some embodiments of a compound of Formula (I), at least one of R_{C} is —C(═O)NR⁴R⁵.

In some embodiments of a compound of Formula (I), at least one of R_{C} is —C(═O)OR³ and each R³ is independently selected from C₁-C₆ alkyl optionally substituted with one or more of —OH, optionally substituted —OC(═O)C₁-C₆ alkyl, C₁-C₆ alkoxy, —C(═O)OH, and —NR¹R²; wherein the —OC(═O)C₁-C₆ alkyl is optionally substituted with one or more of —OH and —NR⁷R⁸. In some embodiments of a compound of Formula (I), at least one of R_{C} is —C(═O)OR³ and each R³ is independently selected from C₁-C₆ alkyl (optionally substituted with one or more of —OH, C₁-C₆ alkoxy, and —NR¹R²) or —C₁-C₆ alkylene-OC(═O)C₁-C₆ alkyl (wherein C₁-C₆ alkyl is optionally substituted with one or more of —OH and —NR⁷R⁸). In some embodiments of a compound of Formula (I), at least one of R_{C} is —C(═O)OR³ and each R³ is independently C₁-C₆alkyl optionally substituted with one or more of —OH, C₁-C₆ alkoxy, and —NR¹R². In some embodiments of a compound of Formula (I), at least one of R_{C} is —C(═O)OR³ and each R³ is independently selected from

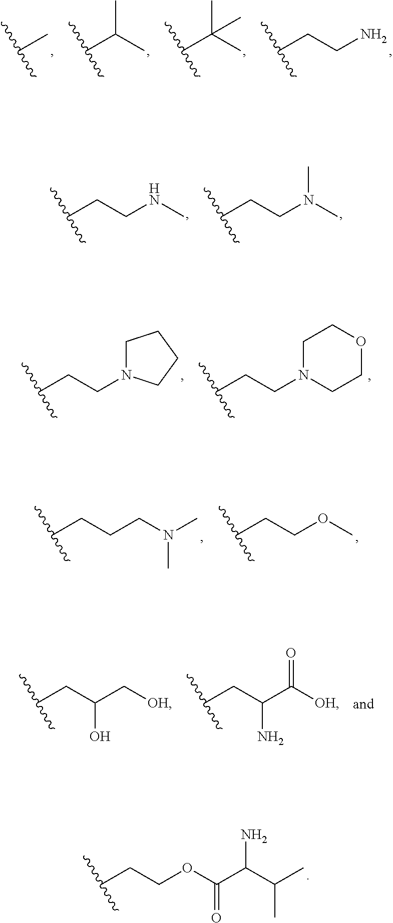

In some embodiments of a compound of Formula (I), at least one of R_{C} is —C(═O)NR⁴R⁵ and each R⁴ and R⁵ is independently selected from hydrogen and C₁-C₆ alkyl; or R⁴ and R⁵ are taken together with the N to which they are attached to form a C₂-C₈ heterocycloalkyl, optionally substituted with one or more C₁-C₆ alkyl substituents. In some embodiments of a compound of Formula (I), at least one of R_{C} is —C(═O)NR⁴R⁵ and each R⁴ and R⁵ are hydrogen.

In some embodiments of a compound of Formula (I), Ar_{T} is selected from pyridinyl, pyrimidinyl, pyrazinyl, phenyl, thiophenyl, pyrazolyl, and imidazolyl, wherein Ar_{T} is optionally substituted by one or more substituents independently selected from halogen, —OH, —NR⁷R⁸, —CN, C₁-C₆ alkyl, and C₁-C₆ alkoxy.

In some embodiments of a compound of Formula (I), Ar_{T} is phenyl optionally substituted by one or more substituents selected from halogen, —OH, —NR⁷R⁸, —CN, C₁-C₆ alkyl, and C₁-C₆ alkoxy.

In some embodiments of a compound of Formula (I), each R_{M} is independently selected from hydrogen, halogen, —OH, —CN, C₁-C₆ alkyl, and C₁-C₆ alkoxy. In some embodiments of a compound of Formula (I), one R_{M} is selected from hydrogen, halogen, —OH, —CN, C₁-C₆ alkyl, and C₁-C₆ alkoxy; and each other R_{M} is independently selected from hydrogen and halogen. In some embodiments of a compound of Formula (I), each R_{M} is hydrogen.

In some embodiments of a compound of Formula (I), R_{L} is selected from optionally substituted heteroaryl, —C(═O)OH, —C(═O)OR⁹, —C(═O)NR¹⁰R¹¹, —NHC(═O)R¹², —NHS(═O)₂R¹², and —C(═O)NHS(═O)₂R¹²;

wherein the heteroaryl is optionally substituted with one or more substituents independently selected from —OH, C₁-C₆ alkyl, C₁-C₆ hydroxyalkyl, C₁-C₆ alkoxy, —OC(═O)C₁-C₆ alkyl, (C₁-C₄ alkylene)-O—C(═O)C₁-C₆ alkyl, —C(═O)R¹², aryl, heteroaryl, C₁-C₆ alkyl-(aryl), and C₁-C₆ alkyl-(heteroaryl). In some embodiments of a compound of Formula (I), R_{L} is —C(═O)OR⁹. In some embodiments of a compound of Formula (I), R_{L} is —C(═O)OR⁹ and R⁹ is C₁-C₆ alkylene-OC(═O)C₁-C₆ alkyl, wherein C₁-C₆ alkyl is optionally substituted with one or more of —OH and —NR⁷R⁸. In some embodiments of a compound of Formula (I), R_{L} is —C(═O)OR⁹ and R⁹ is C₁-C₆ alkyl optionally substituted with —NR¹R². In some embodiments of a compound of Formula (I), R_{L} is —C(═O)OR⁹, R⁹ is C₁-C₆ alkyl optionally substituted with —NR¹R², and each R¹ and R² is independently selected from hydrogen or C₁-C₆ alkyl. In some embodiments of a compound of Formula (I), In some embodiments of a compound of Formula (I), R_{L} is —C(═O)OR⁹, R⁹ is C₁-C₆ alkyl optionally substituted with —NR¹R², and each R¹ and R² is hydrogen. In some embodiments of a compound of Formula (I), R_{L} is —C(═O)OR⁹ and R⁹ is selected from

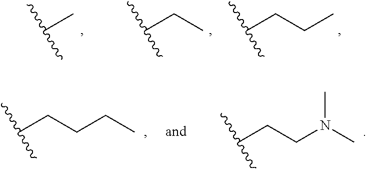

In some embodiments of a compound of Formula (I), R_{L} is —C(═O)NR¹⁰R¹¹, and each R¹⁰ and R¹¹ is independently selected from hydrogen and C₁-C₆ alkyl optionally substituted with one or more substituents independently selected from —C(═O)OH, —C(═O)NR¹R², —OH, aryl, and heteroaryl; or R¹⁰ and R¹¹ are taken together with the N to which they are attached to form a C₂-C₈ heterocycloalkyl, optionally substituted with one or more C₁-C₆ alkyl substituents. In some embodiments of a compound of Formula (I), R_{L} is —C(═O)NR¹⁰R¹¹; R¹⁰ is hydrogen; and R¹¹ is selected from hydrogen,

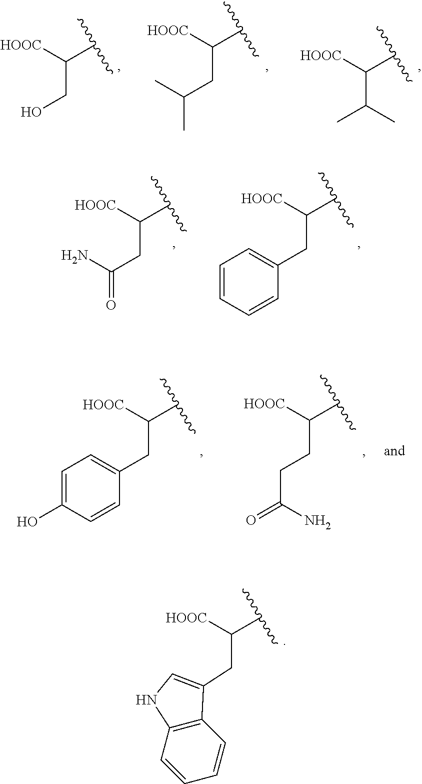

In some embodiments of a compound of Formula (I), R_{L} is selected from —NHC(═O)R¹², —NHS(═O)₂R¹², and —C(═O)NHS(═O)₂R¹², and R¹² is selected from C₁-C₆ alkyl and aryl optionally substituted with one or more C₁-C₆ alkyl substituents. In some embodiments of a compound of Formula (I), R_{L} is —NHC(═O)R¹²; and R¹² is methyl. In some embodiments of a compound of Formula (I), R_{L} is —NHS(═O)₂R¹²; and R¹² is selected from phenyl, tolyl, and methyl. In some embodiments of a compound of Formula (I), R_{L} is —C(═O)NHS(═O)₂R¹²; and R¹² is selected from methyl, butyl, and phenyl. In some embodiments of a compound of Formula (I), R_{L} is —C(═O)OH. In some embodiments of a compound of Formula (I), R_{L} is monocyclic heteroaryl, optionally substituted with one or more substituents independently selected from —OH, C₁-C₆ alkyl, C₁-C₆ hydroxyalkyl, C₁-C₆ alkoxy, —OC(═O)C₁-C₆ alkyl, (C₁-C₄ alkylene)-O—C(═O)C₁-C₆ alkyl, —C(═O)R¹², aryl, heteroaryl, C₁-C₆ alkyl-(aryl), and C₁-C₆ alkyl-(heteroaryl). In some embodiments of a compound of Formula (I), R_{L} is tetrazolyl. In some embodiments of a compound of Formula (I), R_{L} is triazolyl, optionally substituted with one or more substituents independently selected from —OH, C₁-C₆ alkyl, C₁-C₆ hydroxyalkyl, C₁-C₆ alkoxy, —OC(═O)C₁-C₆ alkyl, (C₁-C₄ alkylene)-O—C(═O)C₁-C₆ alkyl, —C(═O)R¹², aryl, heteroaryl, C₁-C₆ alkyl-(aryl), and C₁-C₆ alkyl-(heteroaryl). In some embodiments of a compound of Formula (I), R_{L} is triazolyl. In some embodiments of a compound of Formula (I), R_{L} is not —OMe. In some embodiments of a compound of Formula (I), each R_{M} is independently selected from hydrogen, halogen, —OH, —CN, C₁-C₆ alkyl, and C₁-C₆ alkoxy. In some embodiments of a compound of Formula (I), one R_{M} is selected from hydrogen, halogen, —OH, —CN, C₁-C₆ alkyl, and C₁-C₆ alkoxy; and each other R_{M} is independently selected from hydrogen and halogen. In some embodiments of a compound of Formula (I), each R_{M} is hydrogen.

In some embodiments of a compound of Formula (I), each R¹ and R² is independently selected from hydrogen and C₁-C₆ alkyl; or R¹ and R² are taken together with the N to which they are attached to form a C₂-C₈ heterocycloalkyl, optionally substituted with one or more C₁-C₆alkyl substituents. In some embodiments of a compound of Formula (I), each R¹, R², R⁷ and R⁸ is independently selected from hydrogen and C₁-C₆ alkyl. In some embodiments of a compound of Formula (I), each R¹ and R⁸ is hydrogen and each R² and R⁷ is independently selected from hydrogen and C₁-C₆ alkyl. In some embodiments of a compound of Formula (I), R¹, R², R⁷ and R⁸ are each hydrogen.

In some embodiments of a compound of Formula (I), the compound or a pharmaceutically acceptable salt thereof is in the form of a prodrug. In some embodiments of a compound of Formula (I), the compound or a pharmaceutically acceptable salt thereof is in the form of a prodrug and the prodrug comprises an ester moiety. In some embodiments of a compound of Formula (I), the compound or a pharmaceutically acceptable salt thereof is in the form of a prodrug and the prodrug comprises an amide moiety.

Also disclosed herein are compounds of Formula (Ia) or Formula (Ib):

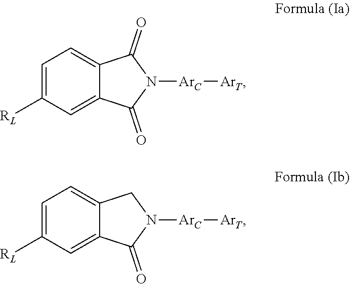

or a pharmaceutically acceptable salt thereof wherein:

Ar_{C} is selected from arylene and heteroarylene; wherein Ar_{C} is substituted with one or more R_{C};

each R_{C} are independently selected from halogen, —CN, optionally substituted C₁-C₆ alkyl, optionally substituted C₁-C₆ alkoxy, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

each R³ is independently optionally substituted C₁-C₆ alkyl;

each R⁴ and R⁵ is independently selected from hydrogen and optionally substituted C₁-C₆ alkyl;

or R⁴ and R⁵ are taken together with the N to which they are attached to form an optionally substituted C₂-C₈ heterocycloalkyl;

each R⁶ are independently selected from optionally substituted C₁-C₆ alkyl and optionally substituted aryl;

Ar_{T} is selected from pyridinyl, pyrimidinyl, pyrazinyl, phenyl, thiophenyl, pyrazolyl, and imidazolyl, wherein Ar_{T} is optionally substituted;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;

R⁹ is optionally substituted C₁-C₆ alkyl;

each R¹⁰ and R¹¹ is independently selected from hydrogen and optionally substituted C₁-C₆ alkyl;

or R¹⁰ and R¹¹ are taken together with the N to which they are attached to form an optionally substituted C₂-C₈ heterocycloalkyl;

R¹² is selected from C₁-C₆ alkyl and optionally substituted aryl;

provided that at least one of R_{C} is not —OH when R_{L} is —C(═O)OH in Formula (Ia) or at least one of R_{C} is not —OEt when R_{L} is —C(═O)OH in Formula (Ia).

In some embodiments of compounds of Formula (Ia) or Formula (Ib):

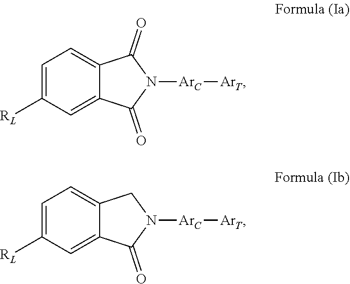

or a pharmaceutically acceptable salt thereof wherein:

Ar_{C} is selected from arylene and heteroarylene; wherein Ar_{C} is substituted with one or more R_{C};

each R_{C} are independently selected from halogen, —CN, optionally substituted C₁-C₆ alkyl, optionally substituted C₁-C₆ alkoxy, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

wherein the C₁-C₆ alkyl and C₁-C₆ alkoxy are optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl; and

wherein the aryl and heteroaryl are optionally substituted with one or more substituents independently selected from —OH, halogen, —C(═O)OH, —C(═O)NR¹R², C₁-C₆ alkyl, C₁-C₆ alkoxy, and —NR⁷R⁸;

each R¹ and R² is independently selected from hydrogen and C₁-C₆ alkyl optionally substituted with one or more substituents independently selected from halogen, —C(═O)OH, —C(═O)NR⁷R⁸, —OH, aryl, heteroaryl, C₃-C₈ cycloalkyl, and C₂-C₈ heterocycloalkyl;

or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R³ is independently C₁-C₆ alkyl optionally substituted with one or more substituents independently selected from —OH, optionally substituted —OC(═O)C₁-C₆ alkyl, optionally substituted —C(═O)OC₁-C₆ alkyl, C₁-C₆ alkoxy, —C(═O)OH, and —NR¹R²; wherein the —OC(═O)C₁-C₆ alkyl and —C(═O)OC₁-C₆ alkyl are optionally substituted with one or more substituents independently selected from —OH and —NR⁷R⁸;

each R⁴ and R⁵ is independently selected from hydrogen and C₁-C₆ alkyl optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl;

or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R⁶ are independently selected from optionally substituted C₁-C₆ alkyl and optionally substituted aryl;

wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and

wherein the aryl is optionally substituted with one or more substituents independently selected from —OH, halogen, —C(═O)OR⁷, —C(═O)NR⁷R⁸, C₁-C₆alkyl, C₁-C₆alkoxy, and —NR⁷R⁸;

each R⁷ and R⁸ is independently selected from hydrogen and C₁-C₆ alkyl;

or R⁷ and R⁸ are taken together with the N to which they are attached to form an optionally substituted C₂-C₈ heterocycloalkyl optionally substituted with one or more C₁-C₆ alkyl substituents;

Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²; wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹R², —C(═O)R¹², aryl, and C₁-C₆ alkyl-(aryl);

R⁹ is C₁-C₆ alkyl optionally substituted with one or more substituent independently selected from —OH and —NR¹R²;

each R¹⁰ and R¹¹ is independently selected from hydrogen and C₁-C₆ alkyl optionally substituted with one or more substituents independently selected from —C(═O)OH, —C(═O)NR¹R², —OH, aryl, hydroxyaryl and heteroaryl;

or R¹⁰ and R¹¹ are taken together with the N to which they are attached to form a C₂-C₈ heterocycloalkyl optionally substituted with one or more C₁-C₆ alkyl substituents;

R¹² is selected from C₁-C₆ alkyl and aryl optionally substituted with one or more C₁-C₆ alkyl substituents;

provided that at least one of R_{C} is not —OH when R_{L} is —C(═O)OH in Formula (Ia) or at least one of R_{C} is not —OEt when R_{L} is —C(═O)OH in Formula (Ia).

In some embodiments of a compound of Formula (Ia) or (Ib), Ar_{C} is arylene substituted with one or two R_{C}. In some embodiments of a compound of Formula (Ia) or (Ib), Ar_{C} is a monocyclic arylene substituted with one or two R_{C}. In some embodiments of a compound of Formula (Ia) or (Ib), Ar_{C} is arylene substituted with one R_{C}. In some embodiments of a compound of Formula (Ia) or (Ib), Ar_{C} is phenylene substituted with one R_{C}. In some embodiments of a compound of Formula (Ia) or (Ib), Ar_{C} is heteroarylene selected from pyridinylene, pyrimidylene, pyrazinylene, and thiophenylene. In some embodiments of a compound of Formula (Ia) or (Ib), Ar_{C} is heteroarylene substituted with one or two R_{C}. In some embodiments of a compound of Formula (Ia) or (Ib), Ar_{C} is a monocyclic heteroarylene substituted with one or two R_{C}. In some embodiments of a compound of Formula (Ia) or (Ib), Ar_{C} is heteroarylene substituted with one R_{C}. In some embodiments of a compound of Formula (Ia) or (Ib), Ar_{C} is thiophenylene substituted with one R_{C}. In some embodiments of a compound of Formula (Ia) or (Ib), Ar_{C} is thiophenylene substituted with two R_{C}.

In some embodiments of a compound of Formula (Ia) or (Ib), each R_{C} are independently selected from —OH, —CN, halogen, C₁-C₆ alkyl, C₁-C₆ alkoxy, C₁-C₆ hydroxyalkyl, heteroaryl, aryl, —C(═O)OH, —C(═O)OR³, and —C(═O)NR⁴R⁵. In some embodiments of a compound of Formula (Ia) or (Ib), each R_{C} are independently selected from —CN, halogen, C₁-C₆ alkyl, C₁-C₆ hydroxyalkyl, heteroaryl, aryl, —C(═O)OH, —C(═O)OR³, and —C(═O)NR⁴R⁵. In some embodiments of a compound of Formula (Ia) or (Ib), one R_{C} is selected from —OH, —CN, C₁-C₆ hydroxyalkyl, heteroaryl, aryl, —C(═O)OH, —C(═O)OR³, and —C(═O)NR⁴R⁵; and a second R_{C} is selected from —OH, halogen, C₁-C₆ alkyl, C₁-C₆ alkoxy, and aryl. In some embodiments of a compound of Formula (Ia) or (Ib), each R_{C} are independently selected from —CN, —C(═O)OH, —C(═O)OR³, and tetrazolyl. In some embodiments of a compound of Formula (Ia) or (Ib), one R_{C} is selected from —CN, —C(═O)OH, —C(═O)OR³, and tetrazolyl; and a second R_{C} is selected from —OH, halogen, C₁-C₆ alkyl, C₁-C₆ alkoxy, and aryl. In some embodiments of a compound of Formula (Ia) or (Ib), at least one of R_{C} is not methyl. In some embodiments of a compound of Formula (Ia) or (Ib), at least one of R_{C} is not ethyl. In some embodiments of a compound of Formula (Ia) or (Ib), at least one of R_{C} is not ethoxy. In some embodiments of a compound of Formula (Ia) or (Ib), at least one of R_{C} is not —OH. In some embodiments of a compound of Formula (Ia) or (Ib), at least one of R_{C} is —CN. In some embodiments of a compound of Formula (Ia) or (Ib), at least one of R_{C} is —C(═O)OH. In some embodiments of a compound of Formula (Ia) or (Ib), at least one of R_{C} is tetrazolyl. In some embodiments of a compound of Formula (Ia) or (Ib), at least one of R_{C} is —C(═O)OR³. In some embodiments of a compound of Formula (Ia) or (Ib), at least one of R_{C} is —C(═O)NR⁴R⁵.

In some embodiments of a compound of Formula (Ia) or (Ib), at least one of R_{C} is —C(═O)OR³ and each R³ is independently C₁-C₆ alkyl optionally substituted with one or more substituent selected from —OH, optionally substituted —OC(═O)C₁-C₆ alkyl, optionally substituted —C(═O)OC₁-C₆ alkyl, C₁-C₆ alkoxy, —C(═O)OH, —NR¹R²; wherein the —OC(═O)C₁-C₆ alkyl and —C(═O)OC₁-C₆ alkyl are optionally substituted with one or more substituent independently selected from —OH and —NR⁷R⁸. In some embodiments of a compound of Formula (Ia) or (Ib), at least one of R_{C} is —C(═O)OR³ and each R³ is independently C₁-C₆ alkyl optionally substituted with one or more substituents independently selected from C₁-C₆ alkoxy and —NR¹R². In some embodiments of a compound of Formula (Ia) or (Ib), at least one of R_{C} is —C(═O)OR³ and each R³ is independently selected from

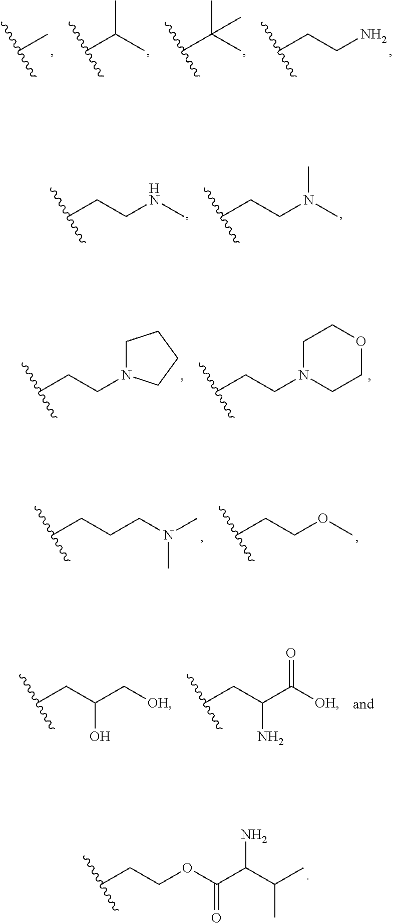

In some embodiments of a compound of Formula (Ia) or (Ib), at least one of R_{C} is —C(═O)ONR⁴R⁵ and each R⁴ and R⁵ is independently selected from hydrogen and C₁-C₆ alkyl; or R⁴ and R⁵ are taken together with the N to which they are attached to form a C₂-C₈ heterocycloalkyl, optionally substituted with one or more C₁-C₆ alkyl substituents. In some embodiments of a compound of Formula (Ia) or (Ib), at least one of R_{C} is —C(═O)ONR⁴R⁵ and each R⁴ and R⁵ is hydrogen.

In some embodiments of a compound of Formula (Ia) or (Ib), Ar_{T} is phenyl optionally substituted by one or more substituents independently selected from halogen, —OH, —NR⁷R⁸, —CN, C₁-C₆ alkyl, and C₁-C₆ alkoxy. In some embodiments of a compound of Formula (Ia) or (Ib), Ar_{T} is selected from pyridinyl, pyrimidinyl, pyrazinyl, thiophenyl, pyrazolyl and imidazolyl, wherein Ar_{T} is optionally substituted by one or more substituents independently selected from halogen, —OH, —NR⁷R⁸, —CN, C₁-C₆ alkyl, and C₁-C₆ alkoxy. In some embodiments of a compound of Formula (Ia) or (Ib), Ar_{T} is selected from thiophenyl, pyrazolyl, and imidazolyl, wherein Ar_{T} is optionally substituted by one or more substituents independently selected from halogen, —OH, —NR⁷R⁸, —CN, C₁-C₆ alkyl, and C₁-C₆ alkoxy. In some embodiments of a compound of Formula (Ia) or (Ib), Ar_{T} is imidazolyl optionally substituted by methyl.

In some embodiments of a compound of Formula (Ia) or (Ib), R_{L} is —C(═O)OR⁹. In some embodiments of a compound of Formula (Ia) or (Ib), R_{L} is —C(═O)OR⁹ and R⁹ is selected from

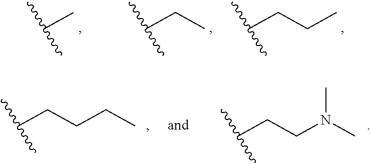

In some embodiments of a compound of Formula (Ia) or (Ib), R_{L} is —C(═O)NR¹⁰R¹¹; R¹⁰ is hydrogen; and R¹¹ is selected from hydrogen,

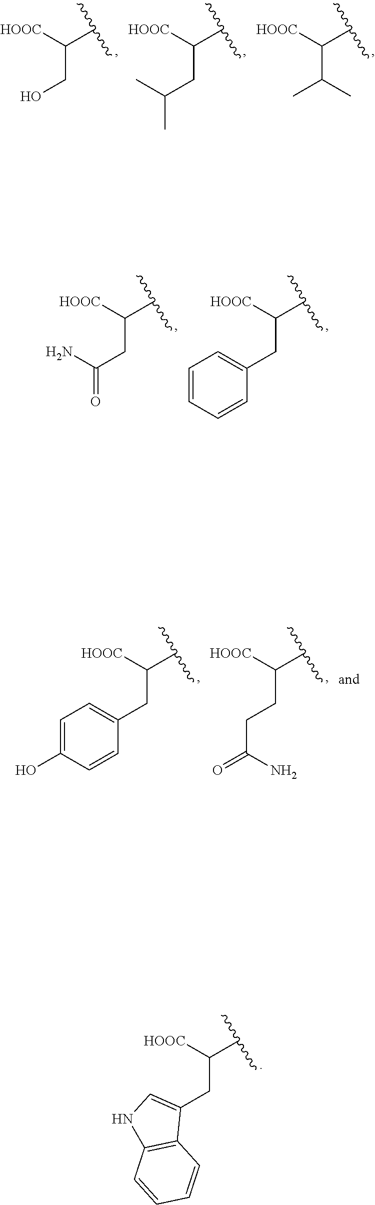

In some embodiments of a compound of Formula (Ia) or (Ib), R_{L} is —NHC(═O)R¹² and R¹² is methyl.

In some embodiments of a compound of Formula (Ia) or (Ib), R_{L} is —NHS(═O)₂R¹² and R¹² is selected from phenyl, toluyl, and methyl. In some embodiments of a compound of Formula (Ia) or (Ib), R_{L} is —C(═O)NHS(═O)R¹². In some embodiments of a compound of Formula (Ia) or (Ib), R¹² is selected from methyl, butyl, and phenyl. In some embodiments of a compound of Formula (Ia) or (Ib), R_{L} is —C(═O)OH. In some embodiments of a compound of Formula (Ia) or (Ib), R_{L} is tetrazolyl. In some embodiments of a compound of Formula (Ia) or (Ib), R_{L} is triazolyl, optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹R², —C(═O)R¹², aryl, and C₁-C₆ alkyl-(aryl). In some embodiments of a compound of Formula (Ia) or (Ib), R_{L} is triazolyl. In some embodiments of a compound of Formula (Ia) or (Ib), R_{L} is not —OMe.

In some embodiments of a compound of Formula (Ia) or (Ib), each R_{M} is independently selected from hydrogen, halogen, —OH, —CN, C₁-C₆ alkyl, and C₁-C₆ alkoxy. In some embodiments of a compound of Formula (Ia) or (Ib), one R_{M} is selected from hydrogen, halogen, —OH, —CN, C₁-C₆ alkyl, and C₁-C₆ alkoxy; and

each other R_{M} is independently selected from hydrogen and halogen. In some embodiments of a compound of Formula (Ia) or (Ib), each R_{M} is hydrogen

In some embodiments of a compound of Formula (Ia) or (Ib), each R¹ and R² is independently selected from hydrogen and C₁-C₆ alkyl, or R¹ and R² are taken together with the N to which they are attached to form an optionally substituted C₂-C₈ heterocycloalkyl optionally substituted with one or more C₁-C₆ alkyl substituents. In some embodiments of a compound of Formula (Ia) or (Ib), each R¹, R², R⁷ and R⁸ is hydrogen.

In some embodiments of a compound of Formula (Ia) or (Ib), the compound or a pharmaceutically acceptable salt thereof is in the form of a prodrug. In some embodiments of a compound of Formula (Ia) or (Ib), the compound or a pharmaceutically acceptable salt thereof is in the form of a prodrug and the prodrug comprises an ester moiety. In some embodiments of a compound of Formula (Ia) or (Ib), the compound or a pharmaceutically acceptable salt thereof is in the form of a prodrug and the prodrug comprises an amide moiety.

Also disclosed herein is a compound of Formula (II):

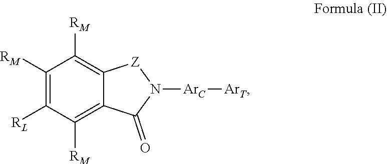

a prodrug thereof, a pharmaceutically acceptable salt thereof, or combination thereof, wherein: Z is —C(═O)— or —C(R^{a})(R^{b})—;

R^{a} and R^{b} are each independently selected from hydrogen, halogen, —OH, C₁₋₆ alkyl, C₁₋₆ alkoxy;

Ar_{C} is selected from arylene and heteroarylene; wherein Ar_{C} is substituted with one or more R_{C};

each R_{C} is independently selected from halogen, —CN, optionally substituted C₁-C₆ alkyl, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

each R³ is independently optionally substituted C₁-C₆ alkyl;

each R⁴ and R⁵ is independently selected from hydrogen and optionally substituted C₁-C₆ alkyl;

or R⁴ and R⁵ are taken together with the N to which they are attached to form an optionally substituted C₂-C₈ heterocycloalkyl;

each R⁶ are independently selected from optionally substituted C₁-C₆ alkyl and optionally substituted aryl;

Ar_{T} is selected from pyridinyl, pyrimidinyl, pyrazinyl, phenyl, thiophenyl, pyrazolyl, and imidazolyl, wherein Ar_{T} is optionally substituted;

each R_{M} is independently selected from hydrogen, halogen, —OH, —CN, optionally substituted C₁-C₆alkyl, and optionally substituted C₁-C₆ alkoxy;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;

R⁹ is C₁-C₆ alkyl optionally substituted with one or more substituents independently selected from —OH and —NR¹R²;

each R¹⁰ and R¹¹ is independently selected from hydrogen and optionally substituted C₁-C₆ alkyl;

or R¹⁰ and R¹¹ are taken together with the N to which they are attached to form an optionally substituted C₂-C₈ heterocycloalkyl;

R¹² is selected from C₁-C₆ alkyl and optionally substituted aryl; and

wherein at least one R_{C} is —C(═O)OH; or R_{L} is —C(═O)OH.

In some embodiments of a compound of Formula (II):

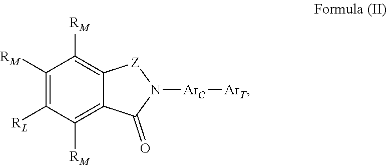

a prodrug thereof, a pharmaceutically acceptable salt thereof, or combination thereof, wherein: Z is —C(═O)— or —C(R^{a})(R^{b})—;

R^{a} and R^{b} are each independently selected from hydrogen, halogen, —OH, C₁₋₆ alkyl, C₁₋₆ alkoxy;

Ar_{C} is selected from arylene and heteroarylene; wherein Ar_{C} is substituted with one or more R_{C};

each R_{C} is independently selected from halogen, —CN, optionally substituted C₁-C₆ alkyl, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, and C₂-C₈ heterocycloalkyl; and

wherein the aryl and heteroaryl are optionally substituted with one or more substituents independently selected from —OH, halogen, —C(═O)OR⁷, —C(═O)NR¹R², C₁-C₆ alkyl, C₁-C₆ alkoxy, and —NR⁷R⁸;

each R¹ and R² is independently selected from hydrogen and C₁-C₆ alkyl optionally substituted with one or more substituents independently selected from halogen, —C(═O)OR⁷, —C(═O)NR⁷R⁸, —OH, aryl, heteroaryl, C₃-C₈ cycloalkyl, and C₂-C₈ heterocycloalkyl;

or R¹ and R² are taken together with the N to which they are attached to form a C₂-C₈ heterocycloalkyl optionally substituted with one or more C₁-C₆ alkyl substituents;

each R³ is independently C₁-C₆ alkyl optionally substituted with one or more substituents independently selected from —OH, optionally substituted —OC(═O)C₁-C₆alkyl, optionally substituted —C(═O)OC₁-C₆ alkyl, C₁-C₆ alkoxy, —C(═O)OH, —NR¹R²;

wherein the —OC(═O)C₁-C₆ alkyl and —C(═O)OC₁-C₆ alkyl are optionally substituted with one or more substituents independently selected from with —OH or —NR⁷R⁸;

each R⁴ and R⁵ is independently selected from hydrogen and C₁-C₆ alkyl optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl;

or R⁴ and R⁵ are taken together with the N to which they are attached to form a C₂-C₈ heterocycloalkyl optionally substituted with one or more C₁-C₆ alkyl substituents;

each R⁶ are independently selected from optionally substituted C₁-C₆ alkyl and optionally substituted aryl;

wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and

wherein the aryl is optionally substituted with one or more substituents independently selected from —OH, halogen, —C(═O)OR⁷, —C(═O)NR⁷R⁸, C₁-C₆ alkyl, C₁-C₆ alkoxy, and —NR⁷R⁸;

each R⁷ and R⁸ is independently selected from hydrogen and C₁-C₆ alkyl;

or R⁷ and R⁸ are taken together with the N to which they are attached to form an optionally substituted C₂-C₈ heterocycloalkyl optionally substituted with one or more C₁-C₆ alkyl substituents;

Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;

each R_{M} is independently selected from hydrogen, halogen, —OH, —CN, optionally substituted C₁-C₆ alkyl, and optionally substituted C₁-C₆ alkoxy;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;

wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹⁰R¹¹, —C(═O)R¹², aryl, or C₁-C₆ alkyl-(aryl);

R⁹ is C₁-C₆ alkyl optionally substituted with one or more substituents independently selected from —OH and —NR¹R²;

each R¹⁰ and R¹¹ is independently selected from hydrogen and C₁-C₆ alkyl optionally substituted with one or more substituents independently selected from —C(═O)OR⁷, —C(═O)NR¹R², —OH, aryl, hydroxyaryl or heteroaryl;

or R¹⁰ and R¹¹ are taken together with the N to which they are attached to form a C₂-C₈ heterocycloalkyl optionally substituted with one or more C₁-C₆ alkyl substituents;

R¹² is selected from C₁-C₆alkyl and aryl optionally substituted with one or more C₁-C₆alkyl substituents; and wherein at least one R_{C} is —C(═O)OH; or R_{L} is —C(═O)OH.

In some embodiments of a compound of Formula (II), Z is —C(═O)—. In some embodiments of a compound of Formula (II), Z is —C(R^{a})(R^{b})—, and R^{a} and R^{b} are each independently selected from hydrogen, fluorine and methyl. In some embodiments of a compound of Formula (II), Z is —CH₂—.

In some embodiments of a compound of Formula (II), Ar_{C} is arylene substituted with one or two R_{C}. In some embodiments of a compound of Formula (II), Ar_{C} is a monocyclic arylene substituted with one or two R_{C}. In some embodiments of a compound of Formula (II), Ar_{C} is arylene substituted with one R_{C}. In some embodiments of a compound of Formula (II), Ar_{C} is phenylene substituted with one R_{C}. In some embodiments of a compound of Formula (II), Ar_{C} is heteroarylene selected from pyridinylene, pyrimidylene, pyrazinylene, and thiophenylene. In some embodiments of a compound of Formula (II), Ar_{C} is heteroarylene substituted with one or two R_{C}. In some embodiments of a compound of Formula (II), Ar_{C} is a monocyclic heteroarylene substituted with one or two R_{C}. In some embodiments of a compound of Formula (II), Ar_{C} is heteroarylene substituted with one R_{C}. In some embodiments of a compound of Formula (II), Ar_{C} is thiophenylene substituted with one R_{C}. In some embodiments of a compound of Formula (II), Ar_{C} is thiophenylene substituted with two R_{C}.

In some embodiments of a compound of Formula (II), each R_{C} are independently selected from —CN, halogen, C₁-C₆ alkyl, C₁-C₆ hydroxyalkyl, heteroaryl, aryl, —C(═O)OH, —C(═O)OR³, and —C(═O)NR⁴R⁵. In some embodiments of a compound of Formula (II), one R_{C} is selected from —CN, C₁-C₆ hydroxyalkyl, heteroaryl, aryl, —C(═O)OH, —C(═O)OR³, and —C(═O)NR⁴R⁵; and a second R_{C} is selected from halogen, C₁-C₆ alkyl, and aryl. In some embodiments of a compound of Formula (II), each R_{C} are independently selected from —CN, —C(═O)OH, —C(═O)OR³, and tetrazolyl. In some embodiments of a compound of Formula (II), one R_{C} is selected from —CN, —C(═O)OH, —C(═O)OR³, and tetrazolyl; and a second R_{C} is selected from halogen, C₁-C₆ alkyl, and aryl. In some embodiments of a compound of Formula (II), at least one of R_{C} is not methyl. In some embodiments of a compound of Formula (II), at least one of R_{C} is not ethyl. In some embodiments of a compound of Formula (II), at least one of R_{C} is —CN. In some embodiments of a compound of Formula (II), at least one of R_{C} is —C(═O)OH. In some embodiments of a compound of Formula (II), at least one of R_{C} is tetrazolyl. In some embodiments of a compound of Formula (II), at least one of R_{C} is —C(═O)OR³. In some embodiments of a compound of Formula (II), at least one of R_{C} is —C(═O)NR⁴R⁵.

In some embodiments of a compound of Formula (II), at least one of R_{C} is —C(═O)OR³ and each R³ is independently C₁-C₆ alkyl optionally substituted with one or more substituent selected from —OH, optionally substituted —OC(═O)C₁-C₆alkyl, optionally substituted —C(═O)OC₁-C₆ alkyl, C₁-C₆ alkoxy, —C(═O)OH, —NR¹R²; wherein the —OC(═O)C₁-C₆ alkyl and —C(═O)OC₁-C₆ alkyl are optionally substituted with one or more substituent independently selected from —OH and —NR⁷R⁸. In some embodiments of a compound of Formula (II), at least one of R_{C} is —C(═O)OR³ and each R³ is independently C₁-C₆ alkyl optionally substituted with one or more substituents independently selected from C₁-C₆ alkoxy and —NR¹R². In some embodiments of a compound of Formula (II), at least one of R_{C} is —C(═O)OR³ and each R³ is independently selected from

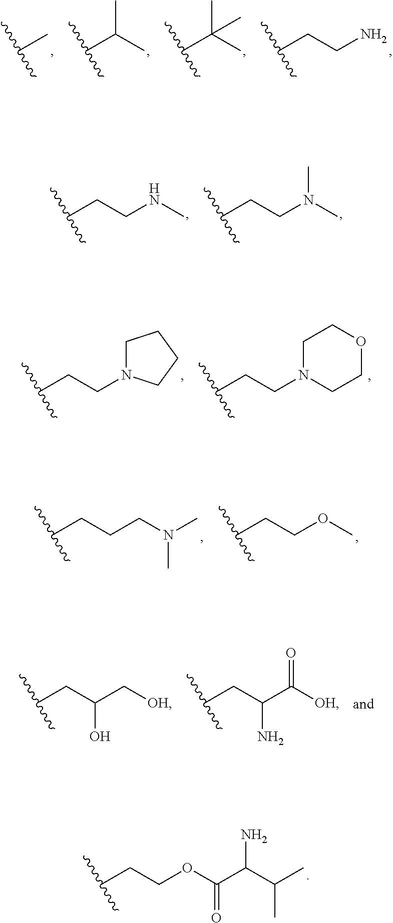

In some embodiments of a compound of Formula (II), at least one of R_{C} is —C(═O)NR⁴R⁵ and each R⁴ and R⁵ is independently selected from hydrogen and C₁-C₆alkyl; or R⁴ and R⁵ are taken together with the N to which they are attached to form a C₂-C₈ heterocycloalkyl, optionally substituted with one or more C₁-C₆ alkyl substituents. In some embodiments of a compound of Formula (II), at least one of R_{C} is —C(═O)NR⁴R⁵ and each R⁴ and R⁵ is hydrogen.

In some embodiments of a compound of Formula (II), Ar_{T} is phenyl optionally substituted by one or more substituents independently selected from halogen, —OH, —NR⁷R⁸, —CN, C₁-C₆ alkyl, and C₁-C₆ alkoxy. In some embodiments of a compound of Formula (II), Ar_{T} is selected from pyridinyl, pyrimidinyl, pyrazinyl, thiophenyl, pyrazolyl and imidazolyl, wherein Ar_{T} is optionally substituted by one or more substituents independently selected from halogen, —OH, —NR⁷R⁸, —CN, C₁-C₆ alkyl, and C₁-C₆ alkoxy. In some embodiments of a compound of Formula (II), Ar_{T} is selected from thiophenyl, pyrazolyl, and imidazolyl, wherein Ar_{T} is optionally substituted by one or more substituents independently selected from halogen, —OH, —NR⁷R⁸, —CN, C₁-C₆ alkyl, and C₁-C₆ alkoxy. In some embodiments of a compound of Formula (II), Ar_{T} is imidazolyl optionally substituted by methyl.

In some embodiments of a compound of Formula (II), R_{L} is —C(═O)OR⁹. In some embodiments of a compound of Formula (II), R_{L} is —C(═O)OR⁹ and R⁹ is selected from

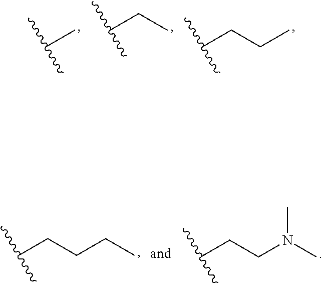

In some embodiments of a compound of Formula (II), R_{L} is —C(═O)NR¹⁰R¹¹; R¹⁰ is hydrogen; and R¹¹ is selected from hydrogen,

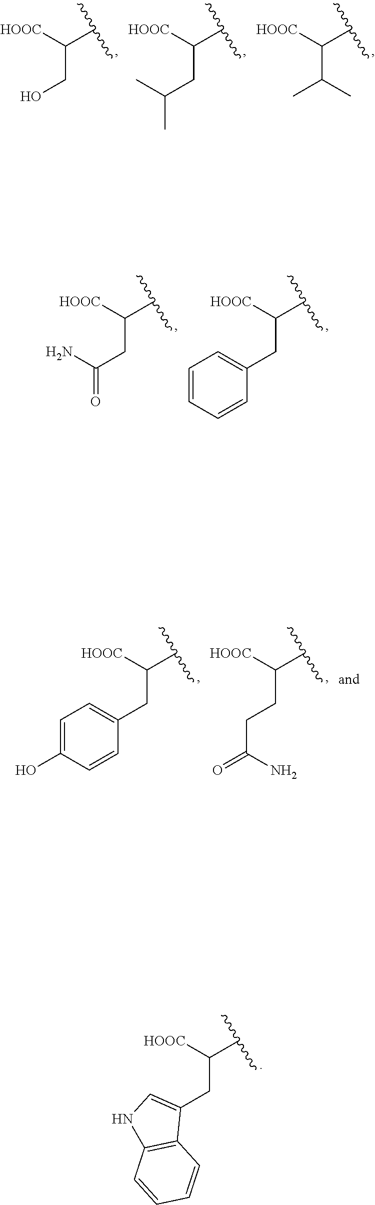

In some embodiments of a compound of Formula (II), R_{L} is —NHC(═O)R¹² and R¹² is methyl. In some embodiments of a compound of Formula (II), R_{L} is —NHS(═O)₂R¹² and R¹² is selected from phenyl, toluyl, and methyl. In some embodiments of a compound of Formula (II), R_{L} is —C(═O)NHS(═O)R¹². In some embodiments of a compound of Formula (II), R_{L} is —C(═O)NHS(═O)R¹² and R¹² is selected from methyl, butyl, and phenyl. In some embodiments of a compound of Formula (II), R_{L} is —C(═O)OH. In some embodiments of a compound of Formula (II), R_{L} is tetrazolyl. In some embodiments of a compound of Formula (II), R_{L} is triazolyl, optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹R², —C(═O)R¹², aryl, and C₁-C₆ alkyl-(aryl). In some embodiments of a compound of Formula (II), R_{L} is triazolyl.

In some embodiments of a compound of Formula (II), each R_{M} is independently selected from hydrogen, halogen, —OH, —CN, C₁-C₆ alkyl, and C₁-C₆ alkoxy. In some embodiments of a compound of Formula (II), one R_{M} is selected from hydrogen, halogen, —OH, —CN, C₁-C₆ alkyl, and C₁-C₆ alkoxy; and each other R_{M} is independently selected from hydrogen and halogen. In some embodiments of a compound of Formula (II), each R_{M} is hydrogen

In some embodiments of a compound of Formula (II), each R¹ and R² is independently selected from hydrogen and C₁-C₆ alkyl, or R¹ and R² are taken together with the N to which they are attached to form an optionally substituted C₂-C₈ heterocycloalkyl optionally substituted with one or more C₁-C₆ alkyl substituents.

In some embodiments of a compound of Formula (II), each R¹, R², R⁷ and R⁸ is hydrogen.

Also disclosed herein is a compound of Formula (III):

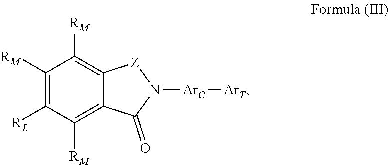

or a pharmaceutically acceptable salt thereof, wherein: Z is —C(═O)— or —C(R^{a})(R^{b})—;

R^{a} and R^{b} are each independently selected from hydrogen, halogen, —OH, C₁₋₆ alkyl, C₁₋₆ alkoxy;

Ar_{C} is selected from arylene and heteroarylene; wherein Ar_{C} is substituted with one or more R_{C};

each R_{C} is independently selected from halogen, —CN, optionally substituted C₁-C₆ alkyl, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

each R³ is independently optionally substituted C₁-C₆ alkyl;

each R⁴ and R⁵ is independently selected from hydrogen and optionally substituted C₁-C₆ alkyl;

or R⁴ and R⁵ are taken together with the N to which they are attached to form an optionally substituted C₂-C₈ heterocycloalkyl;

R⁶ is selected from optionally substituted C₁-C₆ alkyl and optionally substituted aryl;

Ar_{T} is selected from pyridinyl, pyrimidinyl, pyrazinyl, phenyl, thiophenyl, pyrazolyl, and imidazolyl, wherein Ar_{T} is optionally substituted;

each R_{M} is independently selected from hydrogen, halogen, —OH, —CN, optionally substituted C₁-C₆ alkyl, and optionally substituted C₁-C₆ alkoxy;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;

R⁹ is optionally substituted C₁-C₆ alkyl;

each R¹⁰ and R¹¹ is independently selected from hydrogen and optionally substituted C₁-C₆ alkyl;

or R¹⁰ and R¹¹ are taken together with the N to which they are attached to form an optionally substituted C₂-C₈ heterocycloalkyl;

R¹² is selected from C₁-C₆ alkyl and optionally substituted aryl; and

wherein at least one R_{C} is —C(═O)OR³ or R_{L} is —C(═O)OR⁹.

In some embodiments of a compound of Formula (III):

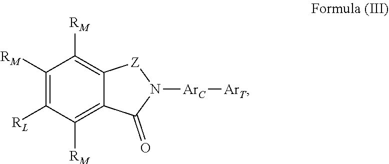

or a pharmaceutically acceptable salt thereof, wherein: Z is —C(═O)— or —C(R^{a})(R^{b})—;

R^{a} and R^{b} are each independently selected from hydrogen, halogen, —OH, C₁₋₆ alkyl, C₁₋₆ alkoxy;

Ar_{C} is selected from arylene and heteroarylene; wherein Ar_{C} is substituted with one or more R_{C};

each R_{C} is independently selected from halogen, —CN, optionally substituted C₁-C₆ alkyl, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, and C₂-C₈ heterocycloalkyl; and

wherein the aryl and heteroaryl are optionally substituted with one or more substituents independently selected from —OH, halogen, —C(═O)OR⁷, —C(═O)NR¹R², C₁-C₆ alkyl, C₁-C₆ alkoxy, and —NR⁷R⁸;

each R¹ and R² is independently selected from hydrogen and C₁-C₆ alkyl optionally substituted with one or more substituents independently selected from halogen, —C(═O)OR⁷, —C(═O)NR⁷R⁸, —OH, aryl, heteroaryl, C₃-C₈ cycloalkyl, and C₂-C₈ heterocycloalkyl;

or R¹ and R² are taken together with the N to which they are attached to form a C₂-C₈ heterocycloalkyl optionally substituted with one or more C₁-C₆ alkyl substituents;

each R³ is independently C₁-C₆ alkyl optionally substituted with one or more substituents independently selected from —OH, optionally substituted —OC(═O)C₁-C₆alkyl, optionally substituted —C(═O)OC₁-C₆ alkyl, C₁-C₆ alkoxy, —C(═O)OH, —NR¹R²;

wherein the —OC(═O)C₁-C₆ alkyl and —C(═O)OC₁-C₆ alkyl are optionally substituted with one or more substituents independently selected from with —OH or —NR⁷R⁸;

each R⁴ and R⁵ is independently selected from hydrogen and C₁-C₆ alkyl optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl;

or R⁴ and R⁵ are taken together with the N to which they are attached to form a C₂-C₈ heterocycloalkyl optionally substituted with one or more C₁-C₆ alkyl substituents;

R⁶ is selected from optionally substituted C₁-C₆ alkyl and optionally substituted aryl;

wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and

wherein the aryl is optionally substituted with one or more substituents independently selected from —OH, halogen, —C(═O)OR⁷, —C(═O)NR⁷R⁸, C₁-C₆alkyl, C₁-C₆alkoxy, and —NR⁷R⁸;

each R⁷ and R⁸ is independently selected from hydrogen and C₁-C₆ alkyl;

or R⁷ and R⁸ are taken together with the N to which they are attached to form an optionally substituted C₂-C₈ heterocycloalkyl optionally substituted with one or more C₁-C₆ alkyl substituents;

Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;

each R_{M} is independently selected from hydrogen, halogen, —OH, —CN, optionally substituted C₁-C₆alkyl, and optionally substituted C₁-C₆ alkoxy;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;

wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹⁰R¹¹, —C(═O)R¹², aryl, or C₁-C₆ alkyl-(aryl);

R⁹ is C₁-C₆ alkyl optionally substituted with one or more substituents independently selected from —OH and —NR¹R²;

each R¹⁰ and R¹¹ is independently selected from hydrogen and C₁-C₆ alkyl optionally substituted with one or more substituents independently selected from —C(═O)OH, —C(═O)NR¹R², —OH, aryl, hydroxyaryl or heteroaryl;

or R¹⁰ and R¹¹ are taken together with the N to which they are attached to form a C₂-C₈ heterocycloalkyl optionally substituted with one or more C₁-C₆ alkyl substituents;

R¹² is selected from C₁-C₆alkyl and aryl optionally substituted with one or more C₁-C₆alkyl substituents; and wherein at least one R_{C} is —C(═O)OR³ or R_{L} is —C(═O)OR⁹.

In some embodiments of a compound of Formula (III), Z is —C(═O)—. In some embodiments of a compound of Formula (III), Z is —C(R^{a})(R^{b})—, and R^{a} and R^{b} are each independently selected from hydrogen, fluorine and methyl. In some embodiments of a compound of Formula (III), Z is —CH₂—.

In some embodiments of a compound of Formula (III), Ar_{C} is arylene substituted with one or two R_{C}. In some embodiments of a compound of Formula (III), Ar_{C} is a monocyclic arylene substituted with one or two R_{C}. In some embodiments of a compound of Formula (III), Ar_{C} is arylene substituted with one R_{C}. In some embodiments of a compound of Formula (III), Ar_{C} is phenylene substituted with one R_{C}. In some embodiments of a compound of Formula (III), Ar_{C} is heteroarylene selected from pyridinylene, pyrimidylene, pyrazinylene, and thiophenylene. In some embodiments of a compound of Formula (III), Ar_{C} is heteroarylene substituted with one or two R_{C}. In some embodiments of a compound of Formula (III), Ar_{C} is a monocyclic heteroarylene substituted with one or two R_{C}. In some embodiments of a compound of Formula (III), Ar_{C} is heteroarylene substituted with one R_{C}. In some embodiments of a compound of Formula (III), Ar_{C} is thiophenylene substituted with one R_{C}. In some embodiments of a compound of Formula (III), Ar_{C} is thiophenylene substituted with two R_{C}.

In some embodiments of a compound of Formula (III), each R_{C} are independently selected from —CN, halogen, C₁-C₆alkyl, C₁-C₆hydroxyalkyl, heteroaryl, aryl, —C(═O)OH, —C(═O)OR³, and —C(═O)NR⁴R⁵. In some embodiments of a compound of Formula (III), one R_{C} is selected from —CN, C₁-C₆ hydroxyalkyl, heteroaryl, aryl, —C(═O)OH, —C(═O)OR³, and —C(═O)NR⁴R⁵; and a second R_{C} is selected from halogen, C₁-C₆ alkyl, and aryl. In some embodiments of a compound of Formula (III), each R_{C} are independently selected from —CN, —C(═O)OH, —C(═O)OR³, and tetrazolyl. In some embodiments of a compound of Formula (III), one R_{C} is selected from —CN, —C(═O)OH, —C(═O)OR³, and tetrazolyl; and a second R_{C} is selected from halogen, C₁-C₆ alkyl, and aryl. In some embodiments of a compound of Formula (III), at least one of R_{C} is not methyl. In some embodiments of a compound of Formula (III), at least one of R_{C} is not ethyl. In some embodiments of a compound of Formula (III), at least one of R_{C} is —CN. In some embodiments of a compound of Formula (III), at least one of R_{C} is —C(═O)OH. In some embodiments of a compound of Formula (III), at least one of R_{C} is tetrazolyl. In some embodiments of a compound of Formula (III), at least one of R_{C} is —C(═O)OR³. In some embodiments of a compound of Formula (III), at least one of R_{C} is —C(═O)NR⁴R⁵.

In some embodiments of a compound of Formula (III), at least one of R_{C} is —C(═O)OR³ and each R³ is independently C₁-C₆ alkyl optionally substituted with one or more substituent selected from —OH, optionally substituted —OC(═O)C₁-C₆ alkyl, optionally substituted —C(═O)OC₁-C₆ alkyl, C₁-C₆ alkoxy, —C(═O)OH, —NR¹R²; wherein the —OC(═O)C₁-C₆ alkyl and —C(═O)OC₁-C₆ alkyl are optionally substituted with one or more substituent independently selected from —OH and —NR⁷R⁸. In some embodiments of a compound of Formula (III), at least one of R_{C} is —C(═O)OR³ and each R³ is independently C₁-C₆ alkyl optionally substituted with one or more substituents independently selected from C₁-C₆ alkoxy and —NR¹R². In some embodiments of a compound of Formula (III), at least one of R_{C} is —C(═O)OR³ and each R³ is independently selected from

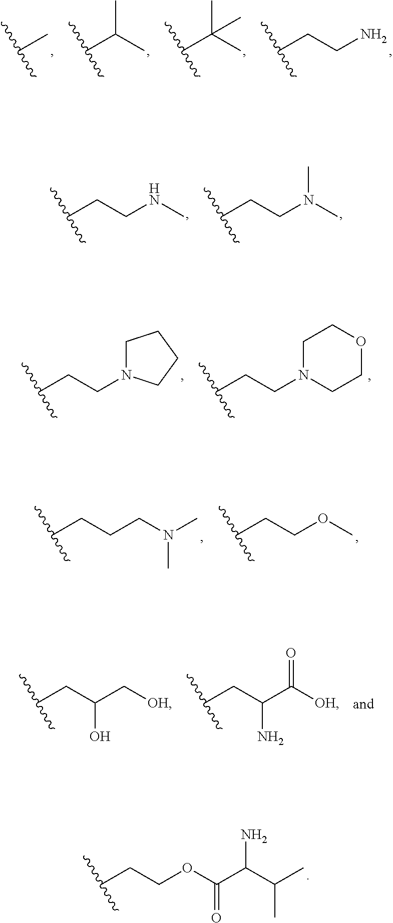

In some embodiments of a compound of Formula (III), at least one of R_{C} is —C(═O)NR⁴R⁵ and each R⁴ and R⁵ is independently selected from hydrogen and C₁-C₆ alkyl; or R⁴ and R⁵ are taken together with the N to which they are attached to form a C₂-C₈ heterocycloalkyl, optionally substituted with one or more C₁-C₆ alkyl substituents. In some embodiments of a compound of Formula (III), at least one of R_{C} is —C(═O)NR⁴R⁵ and each R⁴ and R⁵ is hydrogen.

In some embodiments of a compound of Formula (III), Ar_{T} is phenyl optionally substituted by one or more substituents independently selected from halogen, —OH, —NR⁷R⁸, —CN, C₁-C₆ alkyl, and C₁-C₆ alkoxy. In some embodiments of a compound of Formula (III), Ar_{T} is selected from pyridinyl, pyrimidinyl, pyrazinyl, thiophenyl, pyrazolyl and imidazolyl, wherein Ar_{T} is optionally substituted by one or more substituents independently selected from halogen, —OH, —NR⁷R⁸, —CN, C₁-C₆ alkyl, and C₁-C₆ alkoxy. In some embodiments of a compound of Formula (III), Ar_{T} is selected from thiophenyl, pyrazolyl, and imidazolyl, wherein Ar_{T} is optionally substituted by one or more substituents independently selected from halogen, —OH, —NR⁷R⁸, —CN, C₁-C₆ alkyl, and C₁-C₆ alkoxy. In some embodiments of a compound of Formula (III), Ar_{T} is imidazolyl optionally substituted by methyl.

In some embodiments of a compound of Formula (III), R_{L} is —C(═O)OR⁹. In some embodiments of a compound of Formula (III), R_{L} is —C(═O)OR⁹ and R⁹ is selected from

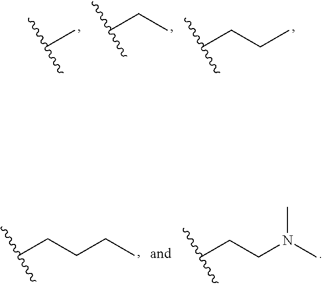

In some embodiments of a compound of Formula (III), R_{L} is —C(═O)NR¹⁰R¹¹; R¹⁰ is hydrogen; and R¹¹ is selected from hydrogen,

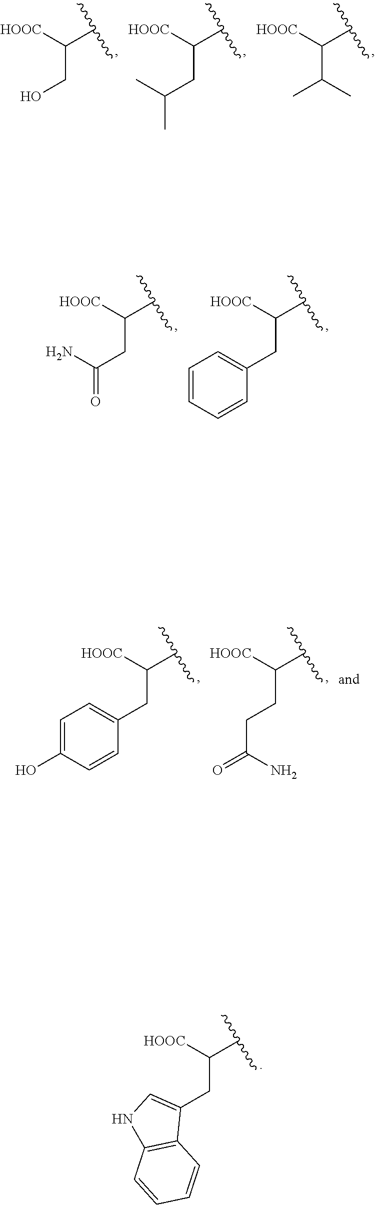

In some embodiments of a compound of Formula (III), R_{L} is —NHC(═O)R¹² and R¹² is methyl. In some embodiments of a compound of Formula (III), R_{L} is —NHS(═O)₂R¹² and R¹² is selected from phenyl, toluyl, and methyl. In some embodiments of a compound of Formula (III), R_{L} is —C(═O)NHS(═O)R¹². In some embodiments of a compound of Formula (III), R_{L} is —C(═O)NHS(═O)R¹² and R¹² is selected from methyl, butyl, and phenyl. In some embodiments of a compound of Formula (III), R_{L} is —C(═O)OH. In some embodiments of a compound of Formula (III), R_{L} is tetrazolyl. In some embodiments of a compound of Formula (III), R_{L} is triazolyl, optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹R², —C(═O)R¹², aryl, and C₁-C₆ alkyl-(aryl). In some embodiments of a compound of Formula (III), R_{L} is triazolyl.

In some embodiments of a compound of Formula (III), each R_{M} is independently selected from hydrogen, halogen, —OH, —CN, C₁-C₆ alkyl, and C₁-C₆ alkoxy. In some embodiments of a compound of Formula (III), one R_{M} is selected from hydrogen, halogen, —OH, —CN, C₁-C₆ alkyl, and C₁-C₆ alkoxy; and each other R_{M} is independently selected from hydrogen and halogen. In some embodiments of a compound of Formula (III), each R_{M} is hydrogen

In some embodiments of a compound of Formula (III), each R¹ and R² is independently selected from hydrogen and C₁-C₆ alkyl, or R¹ and R² are taken together with the N to which they are attached to form an optionally substituted C₂-C₈ heterocycloalkyl optionally substituted with one or more C₁-C₆ alkyl substituents. In some embodiments of a compound of Formula (III), each R¹, R², R⁷ and R⁸ is hydrogen.

Also disclosed herein is a compound of Formula (IV):

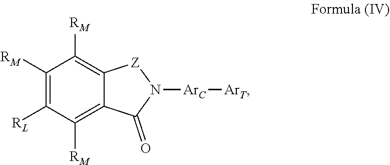

or a pharmaceutically acceptable salt thereof, wherein:

Z is —C(═O)— or —C(R^{a})(R^{b})—;

R^{a} and R^{b} are each independently selected from hydrogen, fluorine and methyl;

Ar_{C} is selected from arylene and heteroarylene; each substituted with one or more R_{C};

R_{C} is selected from —CN, —OH, C₁-C₆ alkoxy, C₁-C₆ alkyl, C₁-C₆ hydroxyalkyl, heteroaryl, aryl, —C(═O)OH, and —C(═O)OR³;

each R³ is independently optionally substituted C₁-C₆ alkyl;

Ar_{T} is selected from pyridinyl, phenyl, thiophenyl, pyrazolyl, imidazolyl, and tetrazolyl, wherein Ar_{T} is optionally substituted;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²;

each R¹⁰ and R¹¹ is independently selected from hydrogen and optionally substituted C₁-C₆ alkyl; and

R¹² is selected from C₁-C₆ alkyl and aryl.

In some embodiments of a compound of Formula (IV):

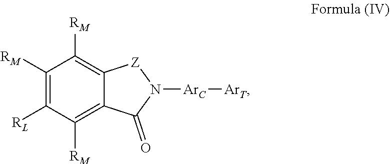

or a pharmaceutically acceptable salt thereof, wherein:

Z is —C(═O)— or —C(R^{a})(R^{b})—;

R^{a} and R^{b} are each independently selected from hydrogen, fluorine and methyl;

Ar_{C} is selected from arylene and heteroarylene; each substituted with one or more R_{C};

R_{C} is selected from —CN, —OH, C₁-C₆ alkoxy, C₁-C₆ alkyl, C₁-C₆ hydroxyalkyl, heteroaryl, aryl, —C(═O)OH, and —C(═O)OR³;

each R¹ and R² is independently selected from hydrogen and C₁-C₆ alkyl;

or R¹ and R² are taken together with the N to which they are attached to form a C₂-C₈ heterocycloalkyl;

each R³ is independently C₁-C₆ alkyl optionally substituted with one or more —NR¹R² or C₁-C₆ alkoxy;

Ar_{T} is selected from pyridinyl, phenyl, thiophenyl, pyrazolyl, imidazolyl, and tetrazolyl, wherein Ar_{T} is optionally substituted by one or more substituents independently selected from halogen, —NR⁷R⁸, C₁-C₆ alkyl, and C₁-C₆ alkoxy;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one or more substituents independently selected from (C₁-C₄ alkylene)-O—C(═O)C₁-C₆ alkyl, —C(═O)NR¹R², —C(═O)R¹², aryl, and C₁-C₆ alkyl-(aryl).

each R¹⁰ and R¹¹ is independently selected from hydrogen and C₁-C₆ alkyl optionally substituted with one or more of —C(═O)OH, —OH, aryl, hydroxyaryl, or heteroaryl; and

R¹² is selected from C₁-C₆ alkyl and aryl.

In some embodiments of a compound of Formula (IV):

Z is —C(═O)— or —CH₂—;

Ar_{C} is selected from phenylene and monocyclic heteroarylene; each substituted with one or more R_{C};

R_{C} is selected from —CN, —OH, C₁-C₆ alkoxy, C₁-C₆ alkyl, heteroaryl, aryl, —C(═O)OH, —C(═O)OR³;

each R¹ and R² is independently selected from hydrogen and C₁-C₆ alkyl;

each R³ is independently C₁-C₆alkyl optionally substituted with one or more —NR¹R²;

Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;

each R_{M} is hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one or more substituents independently selected from —C(═O)R¹² and aryl.

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, aryl, hydroxyaryl, and heteroaryl; and

R¹² is C₁-C₆ alkyl or aryl.

In some embodiments of a compound of Formula (IV):

Z is selected from —C(═O)— and —CH₂—;

Ar_{C} is arylene substituted with one R_{C};

R_{C} is selected from —C(═O)OH and tetrazolyl;

Ar_{T} is selected from pyridinyl, phenyl, thiophenyl, pyrazolyl, imidazolyl, and tetrazolyl, wherein Ar_{T} is optionally substituted by one or more of halogen, C₁-C₆ alkyl, and C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein heteroaryl is optionally substituted by one of —C(═O)R¹² or aryl;

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, phenyl, hydroxyphenyl, and indolyl; and

R¹² is C₁-C₆ alkyl.

In some embodiments of a compound of Formula (IV), Ar_{C} is phenylene.

In some embodiments of a compound of Formula (IV), R_{L} is triazolyl optionally substituted by one of —C(═O)R¹² or aryl.

In some embodiments of a compound of Formula (IV):

Z is selected from —C(═O)— and —CH₂—;

Ar_{C} is heteroarylene substituted with one or two R_{C};

each R_{C} is independently selected from —CN, C₁-C₆ alkyl, and aryl;

Ar_{T} is phenyl optionally substituted by one or more substituents independently selected from halogen, C₁-C₆ alkyl, or C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and C₁-C₆ alkyl optionally substituted by one or more substituents independently selected from —C(═O)OH, —OH, phenyl, hydroxyphenyl, and indolyl; and

R¹² is C₁-C₆ alkyl.

In some embodiments of a compound of Formula (IV), Ar_{C} is thiophenylene.

In some embodiments of a compound of Formula (IV), R_{L} is triazolyl optionally substituted by one of —C(═O)R¹² or aryl.

In some embodiments of a compound of Formula (IV), one of R_{C} is —CN.

In some embodiments of a compound of Formula (IV):

Z is selected from —C(═O)— and —CH₂—;

Ar_{C} is arylene substituted with one R_{C};

R_{C} is —C(═O)OR³;

each R¹ and R² is independently selected from hydrogen and C₁-C₆ alkyl;

R³ is C₁-C₆ alkyl optionally substituted with one NR¹R²;

Ar_{T} is phenyl optionally substituted by one or more substituents independently selected from halogen, C₁-C₆ alkyl, and C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and C₁-C₆ alkyl optionally substituted by one or more substituents independently selected from —C(═O)OH, —OH, phenyl, hydroxyphenyl, and indolyl; and

R¹² is C₁-C₆ alkyl.

In some embodiments of a compound of Formula (IV), Ar_{C} is phenylene.

In some embodiments of a compound of Formula (IV), R_{L} is triazolyl optionally substituted by one of —C(═O)R¹² or aryl.

In some embodiments of a compound of Formula (IV):

Z is —C(═O)—;

Ar_{C} is arylene substituted with one R_{C};

R_{C} is selected from —C(═O)OH and tetrazolyl;

Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and C₁-C₆ alkyl optionally substituted by one or more substituents independently selected from —C(═O)OH, —OH, aryl, hydroxyaryl and heteroaryl; and

R¹² is C₁-C₆ alkyl.

In some embodiments of a compound of Formula (IV), Ar_{C} is phenylene.

In some embodiments of a compound of Formula (IV), R_{L} is triazolyl optionally substituted by one of —C(═O)R¹² or aryl.

In some embodiments of a compound of Formula (IV):

Z is —C(═O)—;

Ar_{C} is heteroarylene substituted with one or two R_{C};

each R_{C} is independently selected from —CN, C₁-C₆ alkyl, and aryl;

Ar_{T} is phenyl optionally substituted by one or more of halogen, C₁-C₆ alkyl, or C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, aryl, hydroxyaryl or heteroaryl; and

R¹² is C₁-C₆ alkyl.

In some embodiments of a compound of Formula (IV), Ar_{C} is thiophenylene.

In some embodiments of a compound of Formula (IV), R_{L} is triazolyl optionally substituted by one of —C(═O)R¹² or aryl.

In some embodiments of a compound of Formula (IV), one of R_{C} is —CN.

In some embodiments of a compound of Formula (IV):

Z is —C(═O)—;

Ar_{C} is arylene substituted with one R_{C};

R_{C} is —C(═O)OR³;

each R¹ and R² is independently selected from hydrogen and C₁-C₆ alkyl;

R³ is C₁-C₆ alkyl optionally substituted with one —NR¹R²;

Ar_{T} is phenyl optionally substituted by one or more of halogen, C₁-C₆ alkyl, or C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

R¹⁰ is selected from hydrogen and C₁-C₆ alkyl;

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, aryl, hydroxyaryl, and heteroaryl; and

R¹² is C₁-C₆ alkyl.

In some embodiments of a compound of Formula (IV), Ar_{C} is phenylene.

In some embodiments of a compound of Formula (IV), R_{L} is triazolyl optionally substituted by one of —C(═O)R¹² or aryl.

Also disclosed herein is a compound of Formula (V):

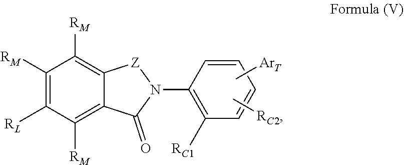

or a pharmaceutically acceptable salt thereof, wherein:

Z is —C(═O)— or —C(R^{a})(R^{b})—;

R^{a} and R^{b} are each independently selected from hydrogen, fluorine and methyl;

R_{C1} is selected from —OH, tetrazolyl, —C(═O)OH, and —C(═O)OR³;

R_{C2} is selected from hydrogen, halogen, —OH, C₁-C₆ alkyl, C₁-C₆ hydroxyalkyl and C₁-C₆ alkoxy;

R³ is optionally substituted C₁-C₆ alkyl;

Ar_{T} is selected from pyridinyl, phenyl, thiophenyl, pyrazolyl, imidazolyl, and tetrazolyl, wherein Ar_{T} is optionally substituted;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²;

R¹⁰ is selected from hydrogen and C₁-C₆ alkyl;

R¹¹ is selected from hydrogen and optionally substituted C₁-C₆ alkyl; and

R¹² is selected from C₁-C₆ alkyl and aryl.

In some embodiments of a compound of Formula (V):

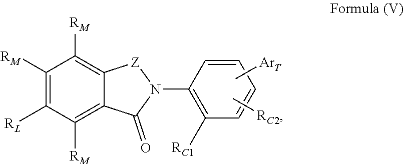

or a pharmaceutically acceptable salt thereof, wherein:

Z is —C(═O)— or —C(R^{a})(R^{b})—;

R^{a} and R^{b} are each independently selected from hydrogen, fluorine and methyl;

R_{C1} is selected from —OH, tetrazolyl, —C(═O)OH, and —C(═O)OR³;

R_{C2} is selected from hydrogen, halogen, —OH, C₁-C₆ alkyl, C₁-C₆ hydroxyalkyl and C₁-C₆ alkoxy;

R³ is C₁-C₆ alkyl optionally substituted with one or more substituent selected from —NR¹R² or C₁-C₆ alkoxy;

each R¹ and R² is independently selected from hydrogen and C₁-C₆ alkyl;

or R¹ and R² are taken together with the N to which they are attached to form a C₂-C₈ heterocycloalkyl;

Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

R¹⁰ is selected from hydrogen and C₁-C₆ alkyl;

R¹¹ is selected from hydrogen and optionally substituted C₁-C₆ alkyl; wherein the C₁-C₆ alkyl is optionally substituted with one or more of —C(═O)OH, —OH, aryl, hydroxyaryl, and heteroaryl; and

R¹² is selected from C₁-C₆ alkyl and aryl.

In some embodiments of a compound of Formula (V), R_{C1} is tetrazolyl or —C(═O)OH.

In some embodiments of a compound of Formula (V), R_{C1} is —C(═O)OR³.

In some embodiments of a compound of Formula (V), R_{C2} is hydrogen.

In some embodiments of a compound of Formula (V), Ar_{T} is selected from pyridinyl, phenyl and thiophenyl, each optionally substituted by one or more substituents independently selected from halogen, C₁-C₆ alkyl, and C₁-C₆ alkoxy.

In some embodiments of a compound of Formula (V), Ar_{T} is pyrazolyl or imidazolyl each optionally substituted by methyl.

In some embodiments of a compound of Formula (V), R_{L} is triazolyl optionally substituted by one of —C(═O)R¹² or aryl.

Also disclosed herein is a compound of Formula (VI):

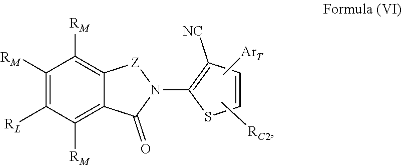

or a pharmaceutically acceptable salt thereof, wherein:

Z is —C(═O)— or —C(R^{a})(R^{b})—;

R^{a} and R^{b} are each independently selected from hydrogen, fluorine and methyl;

R_{C2} is selected from hydrogen, halogen, —OH, C₁-C₆ alkyl, C₁-C₆ hydroxyalkyl, C₁-C₆ alkoxy and aryl;

Ar_{T} is optionally substituted phenyl;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²;

R¹⁰ is selected from hydrogen and C₁-C₆ alkyl;

R¹¹ is selected from hydrogen and optionally substituted C₁-C₆ alkyl; and

R¹² is selected from C₁-C₆ alkyl and aryl.

Also disclosed herein is a compound of Formula (VI):

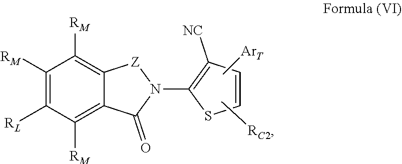

or a pharmaceutically acceptable salt thereof, wherein:

Z is —C(═O)— or —C(R^{a})(R^{b})—;

R^{a} and R^{b} are each independently selected from hydrogen, fluorine and methyl;

R_{C2} is selected from hydrogen, halogen, —OH, C₁-C₆ alkyl, C₁-C₆ hydroxyalkyl, C₁-C₆ alkoxy and aryl;

Ar_{T} is phenyl optionally substituted by one or more substituents independently selected from halogen, C₁-C₆ alkyl, and C₁-C₆ alkoxy;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl.

R¹⁰ is selected from hydrogen and C₁-C₆ alkyl;

R¹¹ is selected from hydrogen and optionally substituted C₁-C₆ alkyl; wherein the C₁-C₆ alkyl is optionally substituted with one or more of —C(═O)OH, —OH, aryl, hydroxyaryl, and heteroaryl; and

R¹² is selected from C₁-C₆ alkyl and aryl.

In some embodiments of a compound of Formula (VI), R_{C2} is selected from C₁-C₆ alkyl and phenyl.

In some embodiments of a compound of Formula (VI), Z is —C(═O)—. In some embodiments of a compound of Formula (VI), Z is —CH₂—.

In some embodiments of a compound of Formula (VI), each R_{M} is hydrogen.

In some embodiments of a compound of Formula (VI), R_{L} is monocyclic heteroaryl optionally substituted by one of —C(═O)R¹² or aryl. In some embodiments of a compound of Formula (VI), R_{L} is tetrazolyl.

In some embodiments of a compound of Formula (VI), R_{L} is triazolyl optionally substituted by one of —C(═O)R¹² or aryl. In some embodiments of a compound of Formula (VI), R_{L} is —C(═O)OH.

In some embodiments of a compound of Formula (VI), R_{L} is —C(═O)NR¹⁰R¹¹ wherein R¹⁰ is selected from hydrogen and C₁-C₆alkyl; and R¹¹ is selected from hydrogen and C₁-C₆alkyl (optionally substituted with one or more of —C(═O)OH, —OH, phenyl, hydroxyphenyl, or indolyl). In some embodiments of a compound of Formula (VI), R_{L} is —C(═O)NHS(═O)₂R¹².

In some embodiments of a compound of Formula (VI), the compound or a pharmaceutically acceptable salt thereof is in the form of a prodrug. In some embodiments of a compound of Formula (VI), the compound or a pharmaceutically acceptable salt thereof is in the form of a prodrug and the prodrug comprises an ester moiety. In some embodiments of a compound of Formula (VI), the compound or a pharmaceutically acceptable salt thereof is in the form of a prodrug and the prodrug comprises an amide moiety.

In some embodiments, the compound disclosed herein has the structure provided in Table 2; in some embodiments the invention provides at least one of the compounds selected from the Table 2

TABLE 2

| Ex. | Name | Structure |
|---|---|---|
| 1 | 2-(2-Methoxybiphenyl-4-yl)-1,3-dioxo- 2,3-dihydro-1H-isoindoline-5-carboxylic acid | 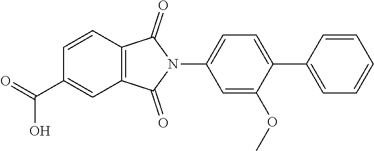 [SMILES: COc1cc(N2C(=O)c3ccc(C(=O)O)cc3C2=O)ccc1-c1ccccc1] |
| 2 | 1,3-Dioxo-2-[3-(1H-tetrazol-5-yl)-1,1′- biphenyl-4-yl]-2,3-dihydro-1H- isoindole-5-carboxylic acid | 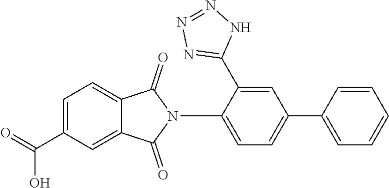 [SMILES: O=C(O)c1ccc2c(c1)C(=O)N(c1ccc(-c3ccccc3)cc1-c1nnn[nH]1)C2=O] |
| 3 | 2-(4-Hydroxy[1,1′-biphenyl]-3-yl)-1,3- dioxo-2,3-dihydro-1H-isoindoline-5- carboxylic acid | 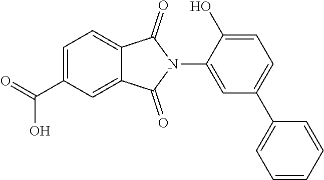 [SMILES: O=C(O)c1ccc2c(c1)C(=O)N(c1cc(-c3ccccc3)ccc1O)C2=O] |
| 4 | 2-(3-Hydroxymethylbiphenyl-4-yl)-1,3- dioxo-2,3-dihydro-1H-isoindole-5- carboxylic acid | 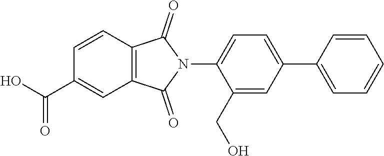 [SMILES: O=C(O)c1ccc2c(c1)C(=O)N(c1ccc(-c3ccccc3)cc1CO)C2=O] |
| 5 | 2-(3-Methoxycarbonylbiphenyl-4-yl)- 1,3-dioxo-2,3-dihydro-1H-isoindole-5- carboxylic acid methy lester | 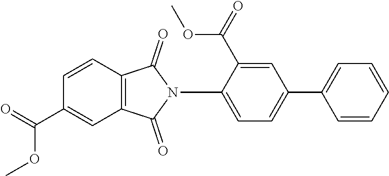 [SMILES: COC(=O)c1ccc2c(c1)C(=O)N(c1ccc(-c3ccccc3)cc1C(=O)OC)C2=O] |
| 6 | 2-[3-Cyano-4-(4-methoxyphenyl)-5- methylthiophen-2-yl]-1,3-dioxo-2,3- dihydro-1H-isoindoline-5-carboxylic acid | 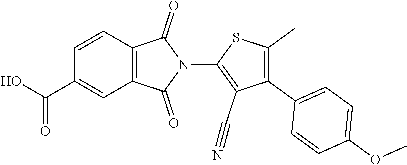 [SMILES: COc1ccc(-c2c(C)sc(N3C(=O)c4ccc(C(=O)O)cc4C3=O)c2C#N)cc1] |
| 7 | 2-[3-Cyano-4-(4-methoxyphenyl)-5- methylthiophen-2-yl]-1,3-dioxo-2,3- dihydro-1H-isoindole-5-carboxylic acid ethyl ester | 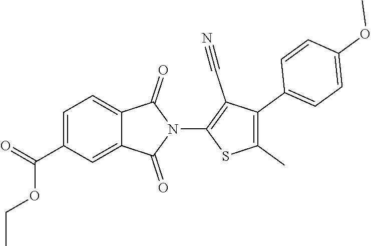 [SMILES: CCOC(=O)c1ccc2c(c1)C(=O)N(c1sc(C)c(-c3ccc(OC)cc3)c1C#N)C2=O] |
| 8 | 2-[3-Cyano-4-(4-methoxyphenyl)-5- methylthiophen-2-yl]-1,3-dioxo-2,3- dihydro-1H-isoindoline-5-carboxylic acid butyl ester | 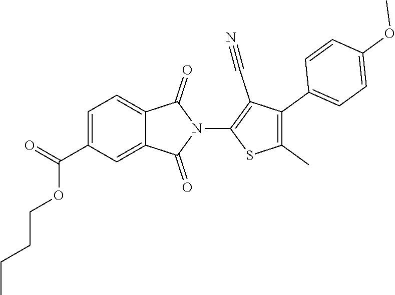 [SMILES: CCCCOC(=O)c1ccc2c(c1)C(=O)N(c1sc(C)c(-c3ccc(OC)cc3)c1C#N)C2=O] |
| 9 | 1,3-Dioxo-2-[3-(1H-tetrazol-5- yl)biphenyl-4-yl]-2,3-dihydro-1H- isoindoline-5-carboxylic acid ethyl ester | 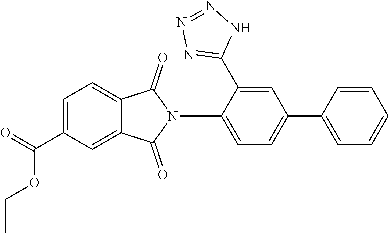 [SMILES: CCOC(=O)c1ccc2c(c1)C(=O)N(c1ccc(-c3ccccc3)cc1-c1nnn[nH]1)C2=O] |
| 10 | 2-(4-Carboxybiphenyl-3-yl)-1,3-dioxo- 2,3-dihydro-1H-isoindoline-5-carboxylic acid | 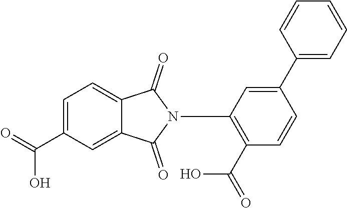 [SMILES: O=C(O)c1ccc2c(c1)C(=O)N(c1cc(-c3ccccc3)ccc1C(=O)O)C2=O] |
| 11 | 2-(4-Hydroxymethylbiphenyl-3-yl)-1,3- dioxo-2,3-dihydro-1H-isoindoline-5- carboxylic acid | 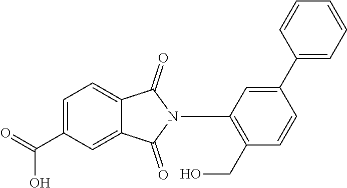 [SMILES: O=C(O)c1ccc2c(c1)C(=O)N(c1cc(-c3ccccc3)ccc1CO)C2=O] |
| 12 | 2-(3-Methoxycarbonylbiphenyl-4-yl)- 1,3-dioxo-2,3-dihydro-1H-isoindoline-5- carboxylic acid | 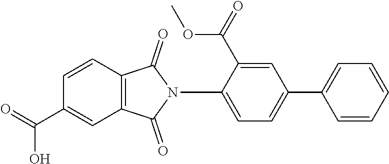 [SMILES: COC(=O)c1cc(-c2ccccc2)ccc1N1C(=O)c2ccc(C(=O)O)cc2C1=O] |
| 13 | N-{2-[3-Cyano-4-(4-methoxyphenyl)- 5-methylthiophen-2-yl]-1,3-dioxo-2,3- dihydro-1H-isoindoline-5-carbonyl}- benzenesulfonamide | 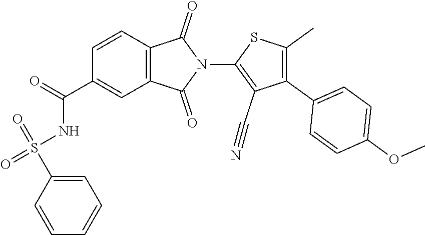 [SMILES: COc1ccc(-c2c(C)sc(N3C(=O)c4ccc(C(=O)NS(=O)(=O)c5ccccc5)cc4C3=O)c2C#N)cc1] |
| 14 | N-{2-[3-Cyano-4-(4-methoxyphenyl)- 5-methylthiophen-2-yl]-1,3-dioxo-2,3- dihydro-1H-isoindoline-5-carbonyl}- methanesulfonamide | 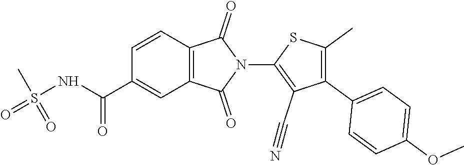 [SMILES: COc1ccc(-c2c(C)sc(N3C(=O)c4ccc(C(=O)NS(C)(=O)=O)cc4C3=O)c2C#N)cc1] |
| 15 | Butane-1-sulfonic acid {2-[3-cyano-4- (4-methoxyphenyl)-5-methylthiophen- 2-yl]-1,3-dioxo-2,3-dihydro-1H- isoindole-5-carbonyl}-amide | 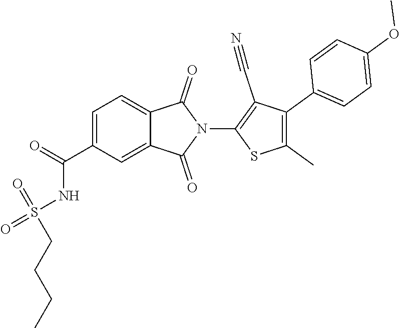 [SMILES: CCCCS(=O)(=O)NC(=O)c1ccc2c(c1)C(=O)N(c1sc(C)c(-c3ccc(OC)cc3)c1C#N)C2=O] |
| 16 | (2S)-2-{[2-(3-cyano-4-(4- methoxyphenyl)-5-methylthiophen-2- yl)-1,3-dioxo-2,3-dihydro-1H- isoindoline-5-carbonyl]amino}-3- hydroxypropanoic acid | 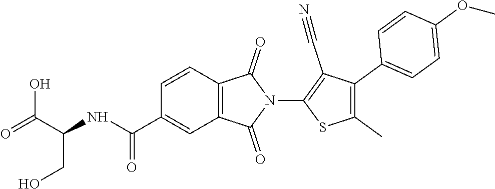 [SMILES: COc1ccc(-c2c(C)sc(N3C(=O)c4ccc(C(=O)N[C@@H](CO)C(=O)O)cc4C3=O)c2C#N)cc1] |
| 17 | (2S)-2-({2-[3-Cyano-4-(4- methoxyphenyl)-5-methylthiophen-2- yl]-1,3-dioxo-2,3-dihydro-1H isoindole-5-carobnyl}amino)-3-(1H- indol-3-yl)propionic acid | 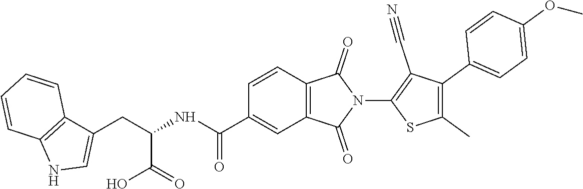 [SMILES: COc1ccc(-c2c(C)sc(N3C(=O)c4ccc(C(=O)N[C@@H](Cc5c[nH]c6ccccc56)C(=O)O)cc4C3=O)c2C#N)cc1] |
| 18 | (2S)-2-({2-[3-Cyano-4-(4- methoxyphenyl)-5-methylthiophen-2- yl]-1,3-dioxo-2,3-dihydro-1H- isoindole-5-carbonyl}-amino)-4- methyl-pentanoic acid | 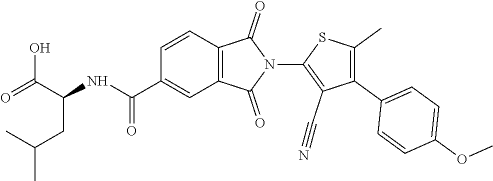 [SMILES: COc1ccc(-c2c(C)sc(N3C(=O)c4ccc(C(=O)N[C@@H](CC(C)C)C(=O)O)cc4C3=O)c2C#N)cc1] |
| 19 | (2S)-2-({2-[3-Cyano-4-(4- methoxyphenyl)-5-methyl-thiophen-2- yl]-1,3-dioxo-2,3-dihydro-1H- isoindoline-5-carbonyl}-amino)-3- methyl-butyric acid | 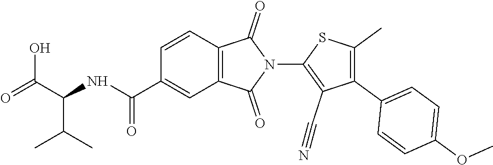 [SMILES: COc1ccc(-c2c(C)sc(N3C(=O)c4ccc(C(=O)N[C@H](C(=O)O)C(C)C)cc4C3=O)c2C#N)cc1] |
| 20 | (2S)-2-({2-[3-Cyano-4-(4- methoxyphenyl)-5-methyl-thiophen-2- yl]-1,3-dioxo-2,3-dihydro-1H- isoindoline-5-carbonyl}-amino)- succinamic acid | 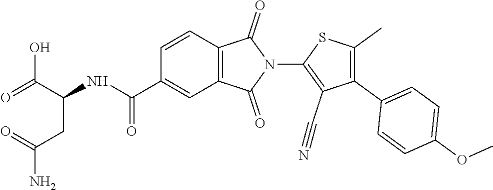 [SMILES: COc1ccc(-c2c(C)sc(N3C(=O)c4ccc(C(=O)N[C@@H](CC(N)=O)C(=O)O)cc4C3=O)c2C#N)cc1] |
| 21 | (2S)-4-Carbamoyl-2-({2-[3-cyano-4- (4-methoxyphenyl)-5-methyl-thiophen- 2-yl]-1,3-dioxo-2,3-dihydro-1H- isoindoline-5-carbonyl}-amino)-butyric acid | 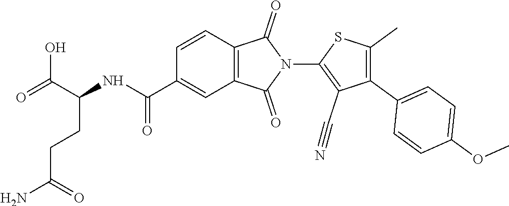 [SMILES: COc1ccc(-c2c(C)sc(N3C(=O)c4ccc(C(=O)N[C@@H](CCC(N)=O)C(=O)O)cc4C3=O)c2C#N)cc1] |
| 22 | 4-(5-Benzenesulfonylaminocarbonyl- 1,3-dioxo-1,3-dihydroisoindol-2- yl)biphenyl-3-carboxylic acid | 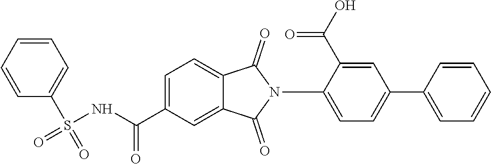 [SMILES: O=C(NS(=O)(=O)c1ccccc1)c1ccc2c(c1)C(=O)N(c1ccc(-c3ccccc3)cc1C(=O)O)C2=O] |
| 23 | 4-(5-Methanesulfonylaminocarbonyl- 1,3-dioxo-1,3-dihydroisoindol-2- yl)biphenyl-3-carboxylic acid | 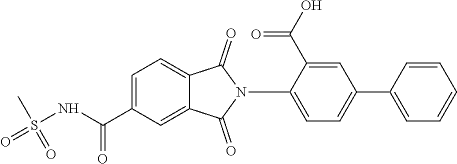 [SMILES: CS(=O)(=O)NC(=O)c1ccc2c(c1)C(=O)N(c1ccc(-c3ccccc3)cc1C(=O)O)C2=O] |
| 24 | 4-[5-(Butane-1- sulfonylaminocarbonyl)-1,3-dioxo-1,3- dihydroisoindol-2-yl]biphenyl-3- carboxylic acid | 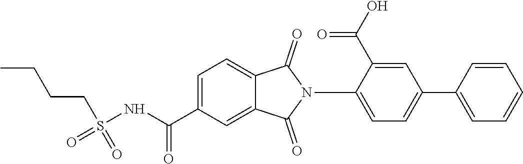 [SMILES: CCCCS(=O)(=O)NC(=O)c1ccc2c(c1)C(=O)N(c1ccc(-c3ccccc3)cc1C(=O)O)C2=O] |
| 25 | 3-(5-Methanesulfonylaminocarbonyl- 1,3-dioxo-1,3-dihydroisoindol-2- yl)biphenyl-4-carboxylic acid | 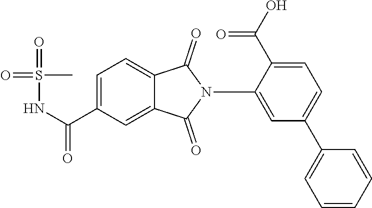 [SMILES: CS(=O)(=O)NC(=O)c1ccc2c(c1)C(=O)N(c1cc(-c3ccccc3)ccc1C(=O)O)C2=O] |
| 26 | 3-[5-(Butane-1- sulfonylaminocarbonyl)-1,3-dioxo-1,3- dihydroisoindol-2-yl]biphenyl-4- carboxylic acid | 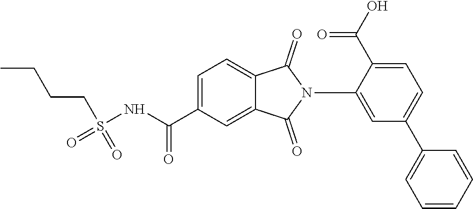 [SMILES: CCCCS(=O)(=O)NC(=O)c1ccc2c(c1)C(=O)N(c1cc(-c3ccccc3)ccc1C(=O)O)C2=O] |
| 27 | 4-(5-Carbamoyl-1,3-dioxo-1,3- dihydroisoindol-2-yl)biphenyl-3- carboxylic acid | 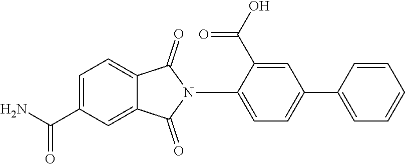 [SMILES: NC(=O)c1ccc2c(c1)C(=O)N(c1ccc(-c3ccccc3)cc1C(=O)O)C2=O] |
| 28 | 3-(5-Carbamoyl-1,3-dioxo-1,3- dihydroisoindol-2-yl)biphenyl-4- carboxylic acid | 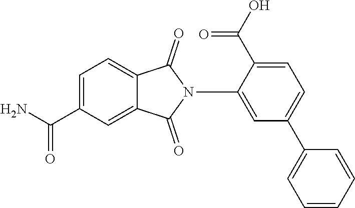 [SMILES: NC(=O)c1ccc2c(c1)C(=O)N(c1cc(-c3ccccc3)ccc1C(=O)O)C2=O] |
| 29 | 4-[5-(1-Benzyl-1H-[1,2,3]triazol-4-yl)- 1,3-dioxo-1,3-dihydroisoindol-2- yl]biphenyl-3-carboxylic acid | 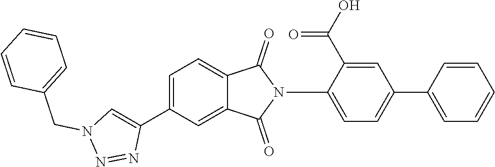 [SMILES: O=C(O)c1cc(-c2ccccc2)ccc1N1C(=O)c2ccc(-c3cn(Cc4ccccc4)nn3)cc2C1=O] |
| 30 | 4-{5-[1-(2,2- Dimethylpropionyloxymethyl)-1H- [1,2,3]triazol-4-yl]-1,3-dioxo-1,3- dihydroisoindol-2-yl}biphenyl-3- carboxylic acid | 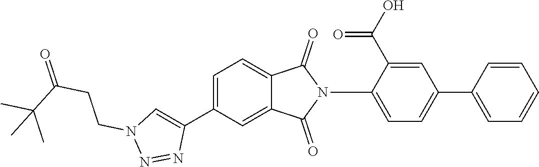 [SMILES: CC(C)(C)C(=O)CCn1cc(-c2ccc3c(c2)C(=O)N(c2ccc(-c4ccccc4)cc2C(=O)O)C3=O)nn1] |
| 31 | 4-[1,3-Dioxo-5-(1H-[1,2,3]triazol-4-yl)- 1,3-dihydroisoindol-2-yl]biphenyl-3- carboxylic acid | 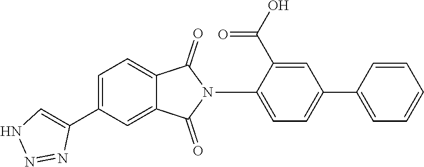 [SMILES: O=C(O)c1cc(-c2ccccc2)ccc1N1C(=O)c2ccc(-c3c[nH]nn3)cc2C1=O] |
| 32 | 2-[1,3-Dioxo-5-(1H-[1,2,3]triazol-4-yl)- 1,3-dihydroisoindol-2-yl]-4-(4- methoxyphenyl)-5-methylthiophene-3- carbonitrile | 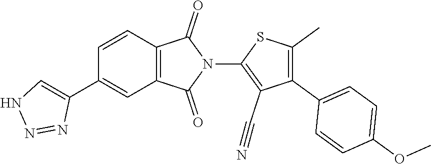 [SMILES: COc1ccc(-c2c(C)sc(N3C(=O)c4ccc(-c5c[nH]nn5)cc4C3=O)c2C#N)cc1] |
| 33 | 4-[1,3-Dioxo-5-(1H-[1,2,3]triazol-4-yl)- 1,3-dihydroisoindol-2-yl]biphenyl-3- carboxylic acid methy lester | 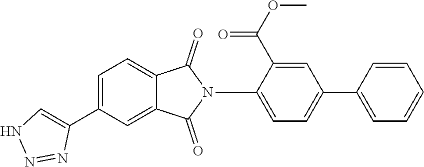 [SMILES: COC(=O)c1cc(-c2ccccc2)ccc1N1C(=O)c2ccc(-c3c[nH]nn3)cc2C1=O] |
| 34 | 3-[1,3-Dioxo-5-(1H-[1,2,3]triazol-4-yl)- 1,3-dihydroisoindol-2-yl]biphenyl-4- carboxylic acid methyl ester | 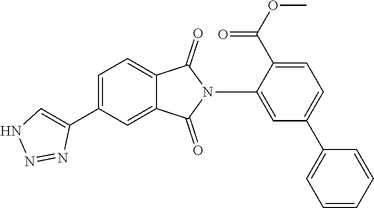 [SMILES: COC(=O)c1ccc(-c2ccccc2)cc1N1C(=O)c2ccc(-c3c[nH]nn3)cc2C1=O] |
| 35 | 2-(3-Methoxycarbonylbiphenyl-4-yl)- 1,3-dioxo-2,3-dihydro-1H-isoindole-5- carboxylic acid butyl ester | 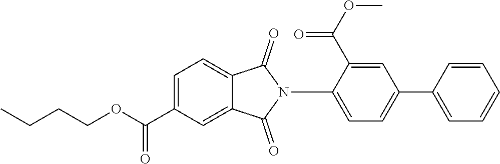 [SMILES: CCCCOC(=O)c1ccc2c(c1)C(=O)N(c1ccc(-c3ccccc3)cc1C(=O)OC)C2=O] |
| 36 | 2-(4-Methoxycarbonylbiphenyl-3-yl)- 1,3-dioxo-2,3-dihydro-1H-isoindole-5- carboxylic acid butyl ester | 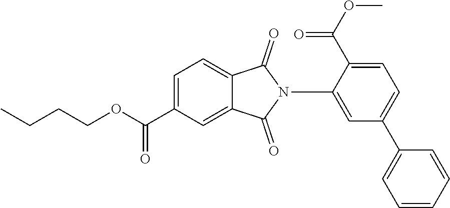 [SMILES: CCCCOC(=O)c1ccc2c(c1)C(=O)N(c1cc(-c3ccccc3)ccc1C(=O)OC)C2=O] |
| 37 | 2-(4-Methoxycarbonylbiphenyl-3-yl)- 1,3-dioxo-2,3-dihydro-1H-isoindole-5- carboxylic acid | 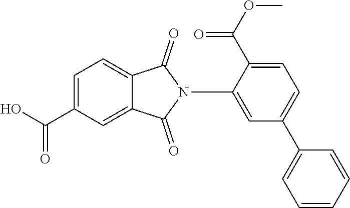 [SMILES: COC(=O)c1ccc(-c2ccccc2)cc1N1C(=O)c2ccc(C(=O)O)cc2C1=O] |
| 38 | 4-[1,3-Dioxo-5-(1H-[1,2,3]triazol-4-yl)- 1,3-dihydroisoindol-2-yl]-6- methoxybiphenyl-3-carboxylic acid methyl ester | 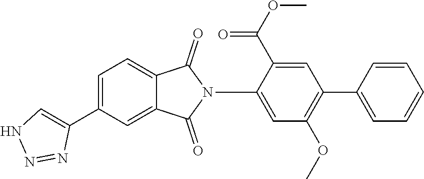 [SMILES: COC(=O)c1cc(-c2ccccc2)c(OC)cc1N1C(=O)c2ccc(-c3c[nH]nn3)cc2C1=O] |
| 39 | 2-(2-Methoxy-5- methoxycarbonylbiphenyl-4-yl)-1,3- dioxo-2,3-dihydro-1H-isoindole-5- carboxylic acid butyl ester | 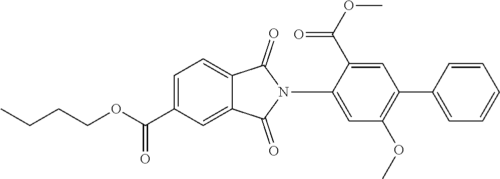 [SMILES: CCCCOC(=O)c1ccc2c(c1)C(=O)N(c1cc(OC)c(-c3ccccc3)cc1C(=O)OC)C2=O] |
| 40 | 4-(5-Methanesulfonylaminocarbonyl0- 1,3-dioxo-1,3-dihydroisoindol-2- yl)biphenyl-3-carboxylic acid methyl ester | 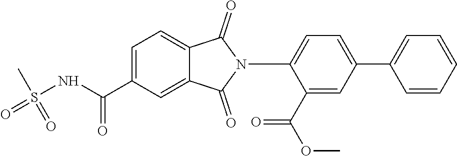 [SMILES: COC(=O)c1cc(-c2ccccc2)ccc1N1C(=O)c2ccc(C(=O)NS(C)(=O)=O)cc2C1=O] |
| 41 | 3-(5-Methanesulfonylaminocarbonyl- 1,3-dioxo-1,3-dihydroisoindol-2- yl)biphenyl-4-carboxylic acid methyl ester | 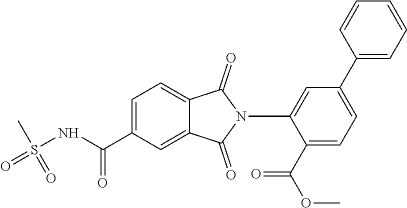 [SMILES: COC(=O)c1ccc(-c2ccccc2)cc1N1C(=O)c2ccc(C(=O)NS(C)(=O)=O)cc2C1=O] |
| 42 | 2-(6-Methoxy-3- methoxycarbonylbiphenyl-4-yl)-1,3- dioxo-2,3-dihydro-1H-isoindole-5- carboxylic acid | 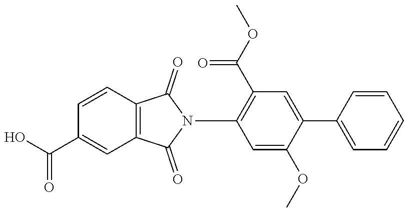 [SMILES: COC(=O)c1cc(-c2ccccc2)c(OC)cc1N1C(=O)c2ccc(C(=O)O)cc2C1=O] |
| 43 | 1,3-Dioxo-2-[4-(1H-tetrazol-5- yl)biphenyl-3-yl]-2,3-dihydro-1H- isoindole-5-carboxylic acid | 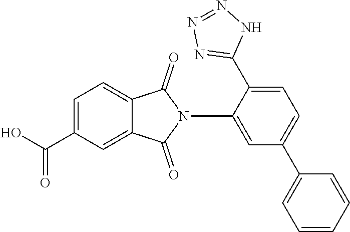 [SMILES: O=C(O)c1ccc2c(c1)C(=O)N(c1cc(-c3ccccc3)ccc1-c1nnn[nH]1)C2=O] |
| 44 | 3-[1,3-Dioxo-5-(1H-tetrazol-5-yl)-1,3- dihydroisoindol-2-yl]biphenyl-4- carboxylic acid methyl ester | 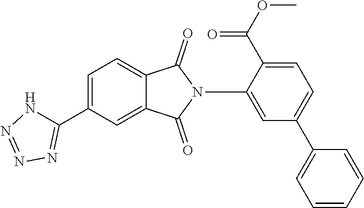 [SMILES: COC(=O)c1ccc(-c2ccccc2)cc1N1C(=O)c2ccc(-c3nnn[nH]3)cc2C1=O] |
| 45 | 2-(4-Carboxy-4′-fluorobiphenyl-3-yl)- 1,3-dioxo-2,3-dihydro-1H-isoindole-5- carboxylic acid | 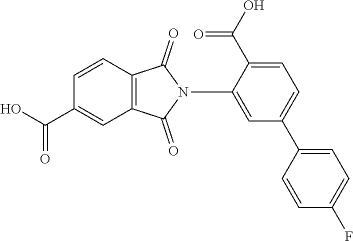 [SMILES: O=C(O)c1ccc2c(c1)C(=O)N(c1cc(-c3ccc(F)cc3)ccc1C(=O)O)C2=O] |
| 46 | 2-(4-Carboxy-3′-fluorobiphenyl-3-yl)- 1,3-dioxo-2,3-dihydro-1H-isoindole-5- carboxylic acid | 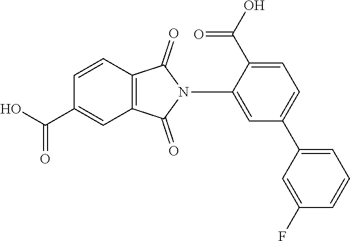 [SMILES: O=C(O)c1ccc2c(c1)C(=O)N(c1cc(-c3cccc(F)c3)ccc1C(=O)O)C2=O] |
| 47 | 2-[5-Methoxy-2-(1H-tetrazol-5- yl)phenyl]-1,3-dioxo-2,3-dihydro-1H- isoindole-5-carboxylic acid | 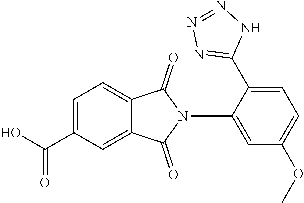 [SMILES: COc1ccc(-c2nnn[nH]2)c(N2C(=O)c3ccc(C(=O)O)cc3C2=O)c1] |
| 48 | 2-(2-Carboxy-5-thiophen-2-yl-phenyl)- 1,3-dioxo-2,3-dihydro-1H-isoindole-5- carboxylic acid | 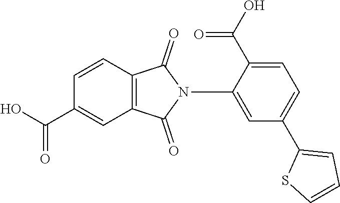 [SMILES: O=C(O)c1ccc2c(c1)C(=O)N(c1cc(-c3cccs3)ccc1C(=O)O)C2=O] |
| 49 | 2-[1,3-Dioxo-5-(1H-[1,2,3]triazol-4-yl)- 1,3-dihydroisoindol-2-yl]-4-pyridin-3- yl-benzoic acid | 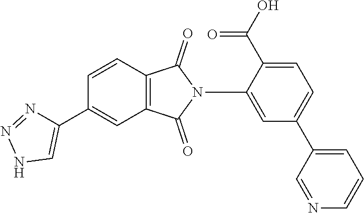 [SMILES: O=C(O)c1ccc(-c2cccnc2)cc1N1C(=O)c2ccc(-c3c[nH]nn3)cc2C1=O] |
| 50 | 3-[1,3-Dioxo-5-(1H-[1,2,3]triazol-4-yl)- 1,3-dihydroisoindol-2-yl]-3′- fluorobiphenyl-4-carboxylic acid | 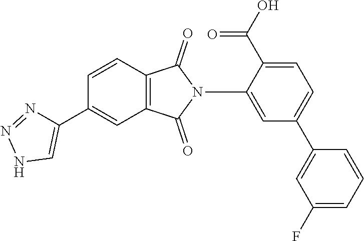 [SMILES: O=C(O)c1ccc(-c2cccc(F)c2)cc1N1C(=O)c2ccc(-c3c[nH]nn3)cc2C1=O] |
| 51 | 3-[1,3-Dioxo-5-(1H-[1,2,3]triazol-4-yl)- 1,3-dihydroisoindol-2-yl]-2′,4′- difluorobiphenyl-4-carboxylic acid | 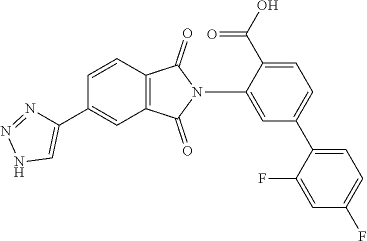 [SMILES: O=C(O)c1ccc(-c2ccc(F)cc2F)cc1N1C(=O)c2ccc(-c3c[nH]nn3)cc2C1=O] |
| 52 | 2-(2-Carboxy-5-pyridin-3-yl-phenyl)- 1,3-dioxo-2,3-dihydro-1H-isoindole-5- carboxylic acid | 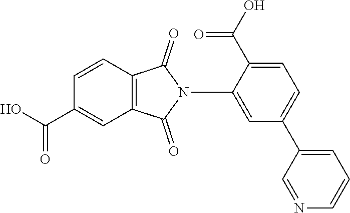 [SMILES: O=C(O)c1ccc2c(c1)C(=O)N(c1cc(-c3cccnc3)ccc1C(=O)O)C2=O] |
| 53 | 2-(4-Carboxy-2′,4′-difluorobiphenyl-3- yl)-1,3-dioxo-2,3-dihydro-1H- isoindole-5-carboxylic acid | 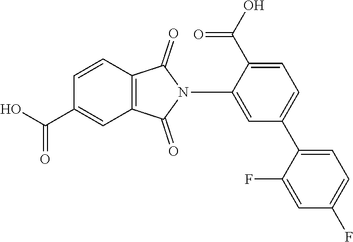 [SMILES: O=C(O)c1ccc2c(c1)C(=O)N(c1cc(-c3ccc(F)cc3F)ccc1C(=O)O)C2=O] |
| 54 | 2-[4-(1H-Tetrazol-5-yl)biphenyl-3-yl]- 5-(1H-[1,2,3]triazol-4-yl)-isoindole-1,3- dione | 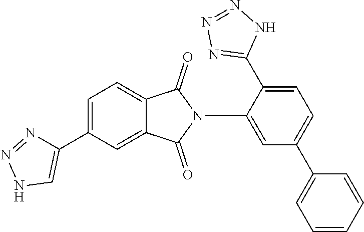 [SMILES: O=C1c2ccc(-c3c[nH]nn3)cc2C(=O)N1c1cc(-c2ccccc2)ccc1-c1nnn[nH]1] |
| 55 | 2-(4-Carboxy-4′-methoxybiphenyl-3- yl)-1,3-dioxo-2,3-dihydro-1H- isoindole-5-carboxylic acid | 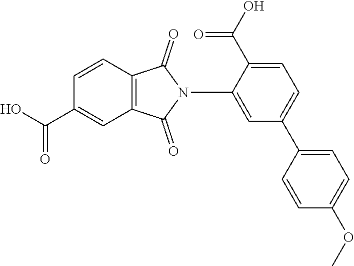 [SMILES: COc1ccc(-c2ccc(C(=O)O)c(N3C(=O)c4ccc(C(=O)O)cc4C3=O)c2)cc1] |
| 56 | 2-[1,3-Dioxo-5-(1H-[1,2,3]triazol-4-yl)- 1,3-dihydroisoindol-2-yl]-4-thiophen-2- yl-benzoic acid | 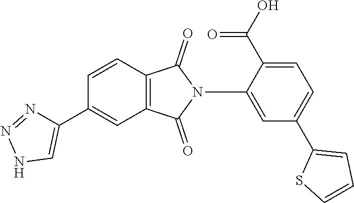 [SMILES: O=C(O)c1ccc(-c2cccs2)cc1N1C(=O)c2ccc(-c3c[nH]nn3)cc2C1=O] |
| 57 | 3-[1,3-Dioxo-5-(1H-[1,2,3]triazol-4-yl)- 1,3-dihydroisoindol-2-yl]-4′- fluorobiphenyl-4-carboxylic acid | 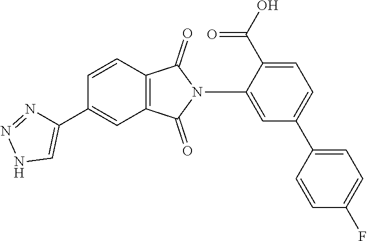 [SMILES: O=C(O)c1ccc(-c2ccc(F)cc2)cc1N1C(=O)c2ccc(-c3c[nH]nn3)cc2C1=O] |
| 58 | 3-[1,3-Dioxo-5-(1H-[1,2,3]triazol-4-yl)- 1,3-dihydroisoindol-2-yl]-4′- methoxybiphenyl-4-carboxylic acid | 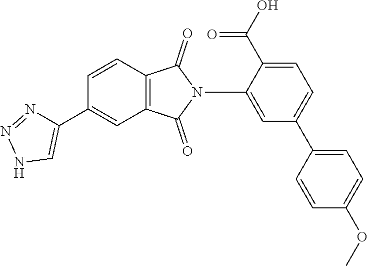 [SMILES: COc1ccc(-c2ccc(C(=O)O)c(N3C(=O)c4ccc(-c5c[nH]nn5)cc4C3=O)c2)cc1] |
| 59 | 3-[1,3-Dioxo-5-(1H-[1,2,3]triazol-4-yl)- 1,3-dihydroisoindol-2-yl]biphenyl-4- carboxylic acid | 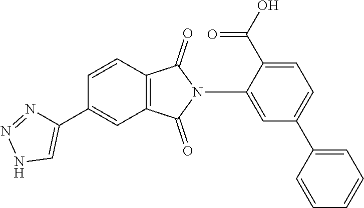 [SMILES: O=C(O)c1ccc(-c2ccccc2)cc1N1C(=O)c2ccc(-c3c[nH]nn3)cc2C1=O] |
| 60 | 2-[1,3-Dioxo-5-(1H-[1,2,3]triazol-4-yl)- 1,3-dihydroisoindol-2-yl]-5-(3H- imidazol-4-yl)-benzoic acid | 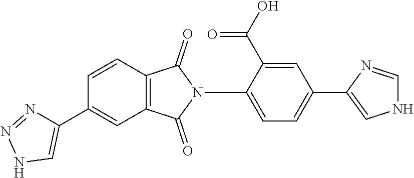 [SMILES: O=C(O)c1cc(-c2c[nH]cn2)ccc1N1C(=O)c2ccc(-c3c[nH]nn3)cc2C1=O] |
| 61 | 2-[1,3-Dioxo-5-(1H-[1,2,3]triazol-4-yl)- 1,3-dihydroisoindol-2-yl]-5-(3-methyl- 3H-imidazol-4-yl)-benzoic acid | 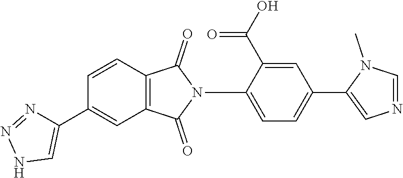 [SMILES: Cn1cncc1-c1ccc(N2C(=O)c3ccc(-c4c[nH]nn4)cc3C2=O)c(C(=O)O)c1] |
| 62 | 2-[1,3-Dioxo-5-(1H-[1,2,3]triazol-4-yl)- 1,3-dihydroisoindol-2-yl]-5-pyridin-3- yl-benzoic acid | 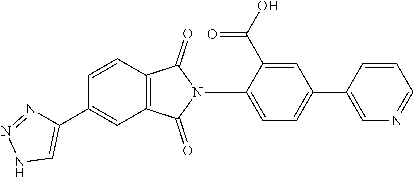 [SMILES: O=C(O)c1cc(-c2cccnc2)ccc1N1C(=O)c2ccc(-c3c[nH]nn3)cc2C1=O] |
| 63 | 2-[1,3-Dioxo-5-(1H-[1,2,3]triazol-4-yl)- 1,3-dihydroisoindol-2-yl]-5-pyrazin-2- yl-benzoic acid | 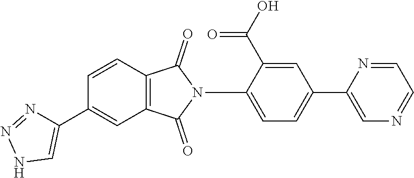 [SMILES: O=C(O)c1cc(-c2cnccn2)ccc1N1C(=O)c2ccc(-c3c[nH]nn3)cc2C1=O] |
| 64 | 2-[1,3-Dioxo-5-(1H-[1,2,3]triazol-4-yl)- 1,3-dihydroisoindol-2-yl]-5-pyrimidin- 5-yl-benzoic acid | 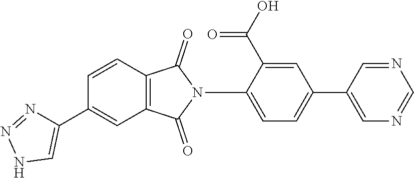 [SMILES: O=C(O)c1cc(-c2cncnc2)ccc1N1C(=O)c2ccc(-c3c[nH]nn3)cc2C1=O] |
| 65 | 5-(6-Amino-pyridin-3-yl)-2-[1,3-dioxo- 5-(1H-[1,2,3]triazol-4-yl)-1,3- dihydroisoindol-2-yl]-benzoic acid | 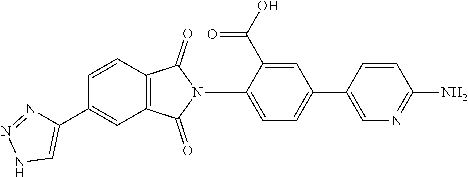 [SMILES: Nc1ccc(-c2ccc(N3C(=O)c4ccc(-c5c[nH]nn5)cc4C3=O)c(C(=O)O)c2)cn1] |
| 66 | 3-[1,3-Dioxo-5-(1H-[1,2,3]triazol-4-yl)- 1,3-dihydroisoindol-2-yl]biphenyl-4- carboxylic acid 2-dimethylamino-ethyl ester | 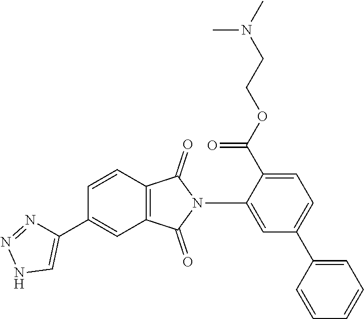 [SMILES: CN(C)CCOC(=O)c1ccc(-c2ccccc2)cc1N1C(=O)c2ccc(-c3c[nH]nn3)cc2C1=O] |
| 67 | 3-[1,3-Dioxo-5-(1H-[1,2,3]triazol-4-yl)- 1,3-dihydroisoindol-2-yl]biphenyl-4- carboyxlic acid 2-morpholin-4-yl-ethyl ester | 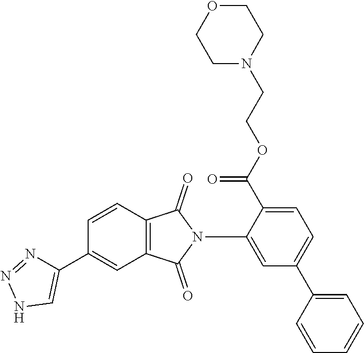 [SMILES: O=C(OCCN1CCOCC1)c1ccc(-c2ccccc2)cc1N1C(=O)c2ccc(-c3c[nH]nn3)cc2C1=O] |
| 68 | 3-[1,3-Dioxo-5-(1H-[1,2,3]triazol-4-yl)- 1,3-dihydroisoindol-2-yl]biphenyl-4- carboxylic acid 2-pyrrolidin-1-yl-ethyl ester | 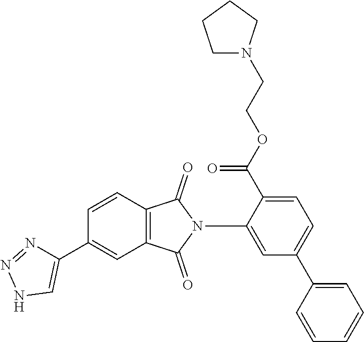 [SMILES: O=C(OCCN1CCCC1)c1ccc(-c2ccccc2)cc1N1C(=O)c2ccc(-c3c[nH]nn3)cc2C1=O] |
| 69 | 3-[1,3-Dioxo-5-(1H-[1,2,3]triazol-4-yl)- 1,3-dihydroisoindol-2-yl]biphenyl-4- carboxylic acid 2-methoxy-ethyl ester | 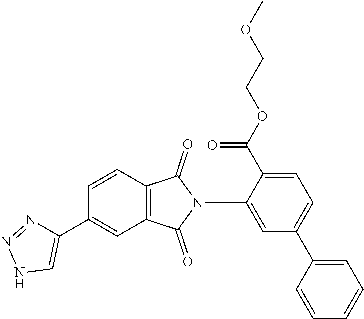 [SMILES: COCCOC(=O)c1ccc(-c2ccccc2)cc1N1C(=O)c2ccc(-c3c[nH]nn3)cc2C1=O] |
| 70 | 3-[1,3-Dioxo-5-(1H-[1,2,3]triazol-4-yl)- 1,3-dihydroisoindol-2-yl]biphenyl-4- carboxylic acid 2,3-dihydroxy-propyl ester | 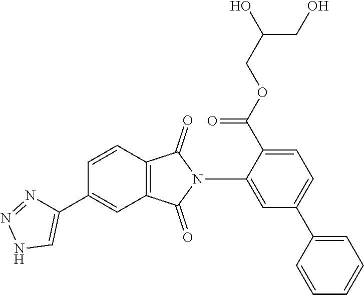 [SMILES: O=C(OCC(O)CO)c1ccc(-c2ccccc2)cc1N1C(=O)c2ccc(-c3c[nH]nn3)cc2C1=O] |
| 71 | 2-[1,3-Dioxo-5-(1H-[1,2,3]triazol-4-yl)- 1,3-dihydroisoindol-2-yl]-5-(1H- pyrazol-4-yl)-benzoic acid | 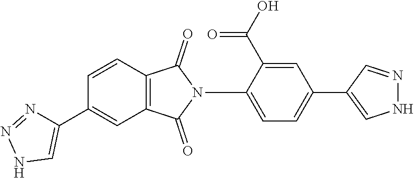 [SMILES: O=C(O)c1cc(-c2cn[nH]c2)ccc1N1C(=O)c2ccc(-c3c[nH]nn3)cc2C1=O] |
| 72 | 3-[1,3-Dioxo-5-(1H-[1,2,3]triazol-4-yl)- 1,3-dihydroisoindol-2-yl]biphenyl-4- carboxylic acid 2-amino-ethyl ester | 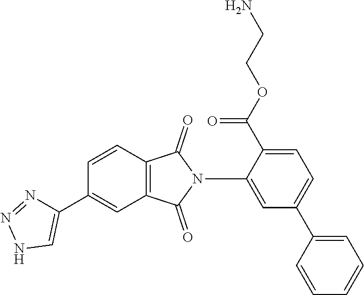 [SMILES: NCCOC(=O)c1ccc(-c2ccccc2)cc1N1C(=O)c2ccc(-c3c[nH]nn3)cc2C1=O] |
| 73 | 3-[1,3-Dioxo-5-(1H-[1,2,3]triazol-4-yl)- 1,3-dihydroisoindol-2-yl]biphenyl-4- carboxylic acid (2S)-2-amino-2- carboxy-ethyl ester | 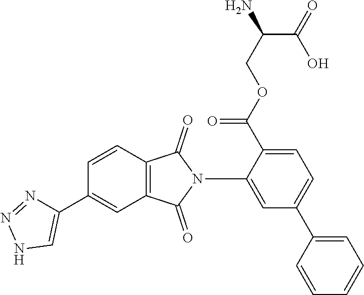 [SMILES: N[C@H](COC(=O)c1ccc(-c2ccccc2)cc1N1C(=O)c2ccc(-c3c[nH]nn3)cc2C1=O)C(=O)O] |
| 74 | 3-[1,3-Dioxo-5-(1H-[1,2,3]triazol-4-yl)- 1,3-dihydroisoindol-2-yl]biphenyl-4- carboxylic acid 2-{[(2S)-2-amino-3- methylbutanoyl]oxy}ethyl ester | 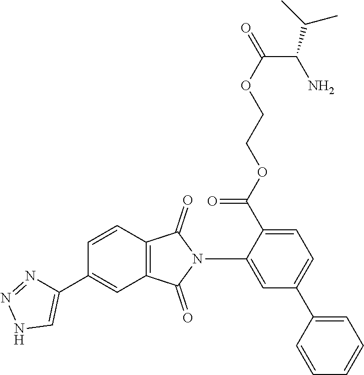 [SMILES: CC(C)[C@H](N)C(=O)OCCOC(=O)c1ccc(-c2ccccc2)cc1N1C(=O)c2ccc(-c3c[nH]nn3)cc2C1=O] |
| 75 | 2-[2-Carboxy-4-(1H-imidazol-4-yl)- phenyl]-1,3-dioxo-2,3-dihydro-1H- isoindole-5-carboxylic acid | 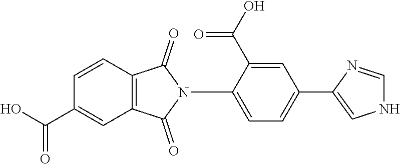 [SMILES: O=C(O)c1ccc2c(c1)C(=O)N(c1ccc(-c3c[nH]cn3)cc1C(=O)O)C2=O] |
| 76 | 2-[2-Carboxy-4-(3-methyl-3H- imidazol-4-yl)-phenyl]-1,3-dioxo-2,3- dihydro-1H-isoindole-5-carboxylic acid | 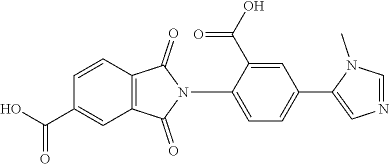 [SMILES: Cn1cncc1-c1ccc(N2C(=O)c3ccc(C(=O)O)cc3C2=O)c(C(=O)O)c1] |
| 77 | 2-[4-(1H-Imidazol-4-yl)-2-(2- methylaminoethoxycarbonyl)-phenyl]- 1,3-dioxo-2,3-dihydro-1H-isoindole-5- carboxylic acid | 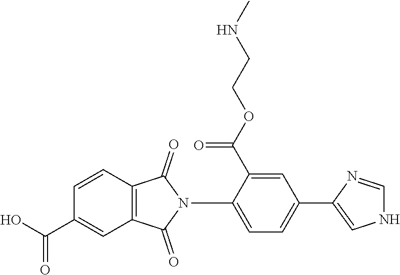 [SMILES: CNCCOC(=O)c1cc(-c2c[nH]cn2)ccc1N1C(=O)c2ccc(C(=O)O)cc2C1=O] |
| 78 | 4-[1,3-Dioxo-5-(1H-[1,2,3]triazol-4-yl)- 1,3-dihydroisoindol-2-yl]biphenyl-3- carboxylic acid 2-dimethylamino-ethyl ester | 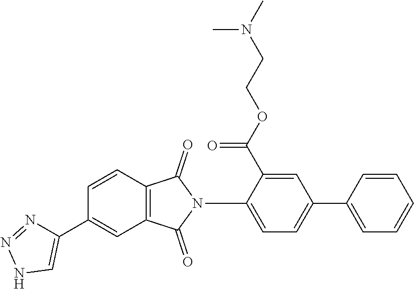 [SMILES: CN(C)CCOC(=O)c1cc(-c2ccccc2)ccc1N1C(=O)c2ccc(-c3c[nH]nn3)cc2C1=O] |
| 79 | 4-[1,3-Dioxo-5-(1H-[1,2,3]triazol-4-yl)- 1,3-dihydroisoindol-2-yl]biphenyl-3- carboxylic acid 2-pyrrolidin-1-yl-ethyl ester | 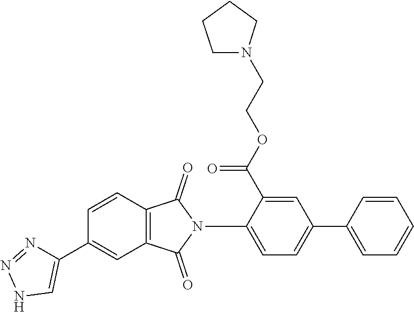 [SMILES: O=C(OCCN1CCCC1)c1cc(-c2ccccc2)ccc1N1C(=O)c2ccc(-c3c[nH]nn3)cc2C1=O] |
| 80 | 4-[1,3-Dioxo-5-(1H-[1,2,3]triazol-4-yl)- 1,3-dihydroisoindol-2-yl]biphenyl-3- carboxylic acid 2-morpholin-4-yl-ethyl ester | 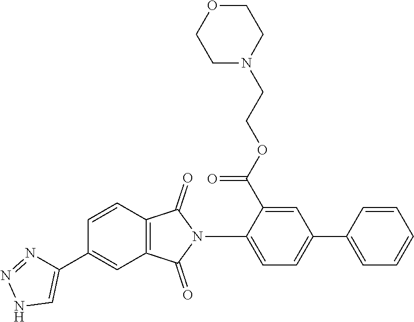 [SMILES: O=C(OCCN1CCOCC1)c1cc(-c2ccccc2)ccc1N1C(=O)c2ccc(-c3c[nH]nn3)cc2C1=O] |
| 81 | 4-[1,3-Dioxo-5-(1H-[1,2,3]triazol-4-yl)- 1,3-dihydroisoindol-2-yl]biphenyl-3- carboxylic acid 2-methylamino-ethyl ester | 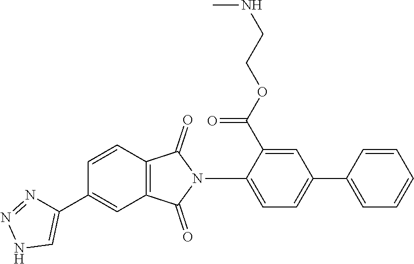 [SMILES: CNCCOC(=O)c1cc(-c2ccccc2)ccc1N1C(=O)c2ccc(-c3c[nH]nn3)cc2C1=O] |
| 82 | 3′,4′-Difluoro-3-[1,3-dioxo-5-(1H- [1,2,3]triazol-4-yl)-1,3-dihydroisoindol- 2-yl]biphenyl-4-carboxylic acid | 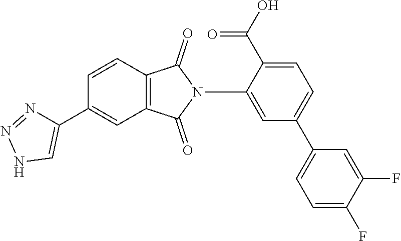 [SMILES: O=C(O)c1ccc(-c2ccc(F)c(F)c2)cc1N1C(=O)c2ccc(-c3c[nH]nn3)cc2C1=O] |
| 83 | 2-(2-Hydroxy-5-phenylpyridin-3-yl)-5- (1H-[1,2,3]triazol-4-yl)-isoindole-1,3- dione | 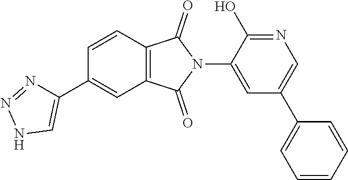 [SMILES: O=C1c2ccc(-c3c[nH]nn3)cc2C(=O)N1c1cc(-c2ccccc2)cnc1O] |
| 84 | 2-(4-Phenylpyridin-2-yl)-5-(1H- [1,2,3]triazol-4-yl)-isoindole-1,3-dione | 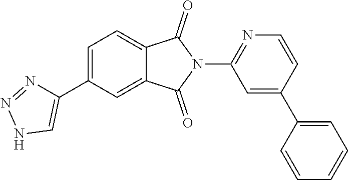 [SMILES: O=C1c2ccc(-c3c[nH]nn3)cc2C(=O)N1c1cc(-c2ccccc2)ccn1] |
| 85 | 2-{1,3-Dioxo-5-(1H-[1,2,3]triazol-4-yl)- 2,3-dihydro-1H-isoindol-2-yl}-4- phenylpyridine N-oxide | 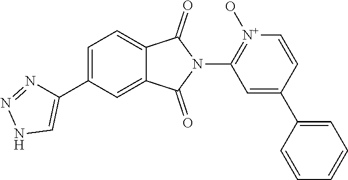 [SMILES: O=C1c2ccc(-c3c[nH]nn3)cc2C(=O)N1c1cc(-c2ccccc2)cc[n+]1O] |
| 86 | 2-(5-Phenylpyridin-2-yl)-5-(1H- [1,2,3]triazol-4-yl)-isoindole-1,3-dione | 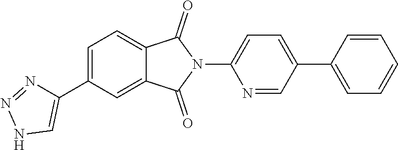 [SMILES: O=C1c2ccc(-c3c[nH]nn3)cc2C(=O)N1c1ccc(-c2ccccc2)cn1] |
| 87 | 2-[4-(4-Fluorophenyl)-pyridin-2-yl]-5- (1H-[1,2,3]triazol-4-yl)-isoindole-1,3- dione | 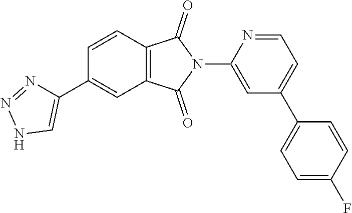 [SMILES: O=C1c2ccc(-c3c[nH]nn3)cc2C(=O)N1c1cc(-c2ccc(F)cc2)ccn1] |
| 88 | 2-(6-Methyl-4-phenylpyridin-2-yl)-5- (1H-[1,2,3]triazol-4-yl)-isoindole-1,3- dione | 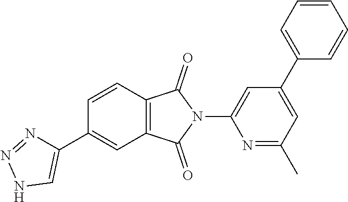 [SMILES: Cc1cc(-c2ccccc2)cc(N2C(=O)c3ccc(-c4c[nH]nn4)cc3C2=O)n1] |
| 89 | 2-(2-Hydroxy-6-phenylpyridin-3-yl)-5- (1H-[1,2,3]triazol-4-yl)-isoindole-1,3- dione | 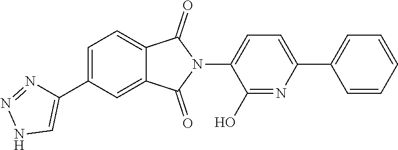 [SMILES: O=C1c2ccc(-c3c[nH]nn3)cc2C(=O)N1c1ccc(-c2ccccc2)nc1O] |
| 90 | 2-[4-(2,4-Difluoro-phenyl)-5-methyl- pyridin-2-yl]-5-(1H-[1,2,3]triazol-4-yl)- isoindole-1,3-dione | 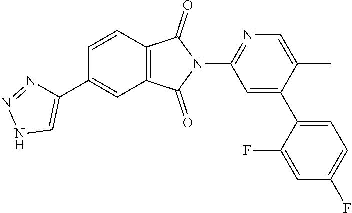 [SMILES: Cc1cnc(N2C(=O)c3ccc(-c4c[nH]nn4)cc3C2=O)cc1-c1ccc(F)cc1F] |
| 91 | 3-[1-Oxo-6-(1H-[1,2,3]triazol-4-yl)-1,3- dihydroisoindol-2-yl]biphenyl-4- carboxylic acid methy lester | 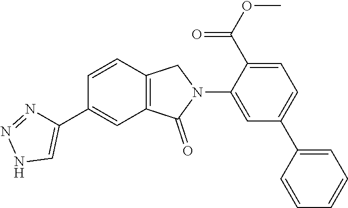 [SMILES: COC(=O)c1ccc(-c2ccccc2)cc1N1Cc2ccc(-c3c[nH]nn3)cc2C1=O] |
| 92 | 3-[1-Oxo-6-(1H-[1,2,3]triazol-4-yl)-1,3- dihydroisoindol-2-yl]biphenyl-4- carboxylic acid | 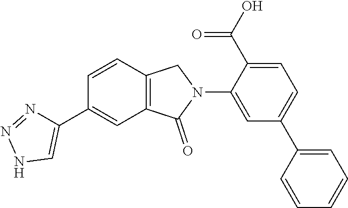 [SMILES: O=C(O)c1ccc(-c2ccccc2)cc1N1Cc2ccc(-c3c[nH]nn3)cc2C1=O] |
| 93 | 3-[1,3-Dioxo-5-(1H-[1,2,3]triazol-4-yl)- 1,3-dihydroisoindol-2-yl]biphenyl-4- carboxylic acid amide | 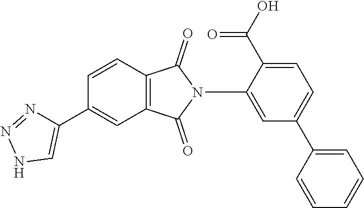 [SMILES: O=C(O)c1ccc(-c2ccccc2)cc1N1C(=O)c2ccc(-c3c[nH]nn3)cc2C1=O] |
| 94 | 4-[1,3-Dioxo-5-(1H-tetrazol-5-yl)-1,3- dihydroisoindol-2-yl]biphenyl-3- carboxylic acid | 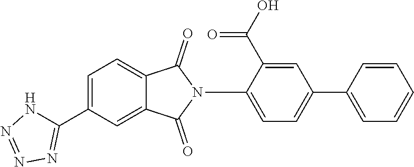 [SMILES: O=C(O)c1cc(-c2ccccc2)ccc1N1C(=O)c2ccc(-c3nnn[nH]3)cc2C1=O] |
| 95 | 3-[1,3-Dioxo-5-(1H-tetrazol-5-yl)-1,3- dihydroisoindol-2-yl]biphenyl-4- carboxylic acid | 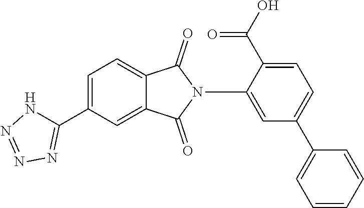 [SMILES: O=C(O)c1ccc(-c2ccccc2)cc1N1C(=O)c2ccc(-c3nnn[nH]3)cc2C1=O] |
| 96 | 3-[1,3-Dioxo-5-(1H-tetrazol-5-yl)-1,3- dihydroisoindol-2-yl]biphenyl-4- carboxylic acid isopropyl ester | 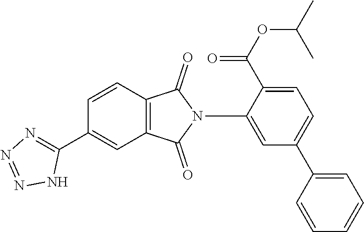 [SMILES: CC(C)OC(=O)c1ccc(-c2ccccc2)cc1N1C(=O)c2ccc(-c3nnn[nH]3)cc2C1=O] |
| 97 | 3-[1,3-Dioxo-5-(1H-tetrazol-5-yl)-1,3- dihydroisoindol-2-yl]biphenyl-4- carboxylic acid 2-dimethylamino-ethyl ester | 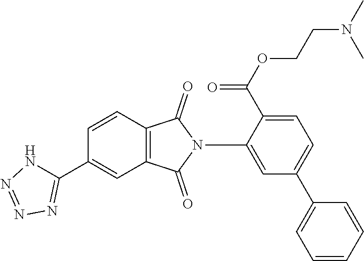 [SMILES: CN(C)CCOC(=O)c1ccc(-c2ccccc2)cc1N1C(=O)c2ccc(-c3nnn[nH]3)cc2C1=O] |
| 98 | 3-[1,3-Dioxo-5-(1H-tetrazol-5-yl)-1,3- dihydroisoindol-2-yl]biphenyl-4- carboxylic acid amide | 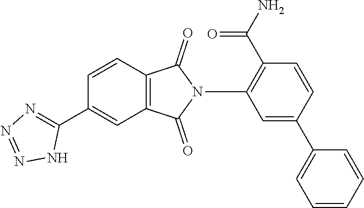 [SMILES: NC(=O)c1ccc(-c2ccccc2)cc1N1C(=O)c2ccc(-c3nnn[nH]3)cc2C1=O] |
| 99 | 3′,4′-Difluoro-3-[1-oxo-6-(1H-tetrazol- 5-yl)-1,3-dihydroisoindol-2-yl]biphenyl- 4-carboxylic acid methyl ester | 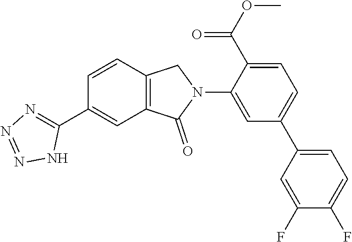 [SMILES: COC(=O)c1ccc(-c2ccc(F)c(F)c2)cc1N1Cc2ccc(-c3nnn[nH]3)cc2C1=O] |
| 100 | 3′,4′-Difluoro-3-[1-oxo-6-(1H-tetrazol- 5-yl)-1,3-dihydroisoindol-2-yl]biphenyl- 4-carboxylic acid | 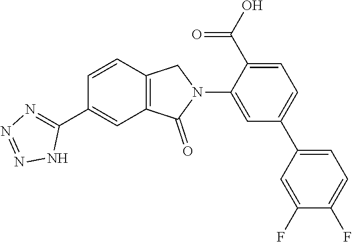 [SMILES: O=C(O)c1ccc(-c2ccc(F)c(F)c2)cc1N1Cc2ccc(-c3nnn[nH]3)cc2C1=O] |
| 101 | 3′,4′-Difluoro-3-[1-oxo-6-(1H-tetrazol- 5-yl)-1,3-dihydroisoindol-2-yl]biphenyl- 4-carboxylic acid amide | 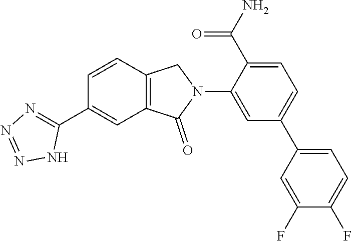 [SMILES: NC(=O)c1ccc(-c2ccc(F)c(F)c2)cc1N1Cc2ccc(-c3nnn[nH]3)cc2C1=O] |
| 102 | 2-(4-Methoxycarbonylbiphenyl-3yl)-3- oxo-2,3-dihydro-1H-isoindole-5- carboxylic acid | 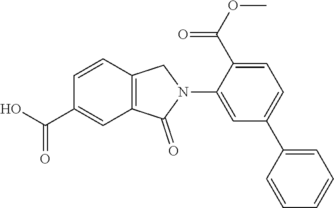 [SMILES: COC(=O)c1ccc(-c2ccccc2)cc1N1Cc2ccc(C(=O)O)cc2C1=O] |
| 103 | 3-(6-Methanesulfonylaminocarbonyl- 1-oxo-1,3-dihydroisoindol-2- yl)biphenyl-4-carboxylic acid methyl ester | 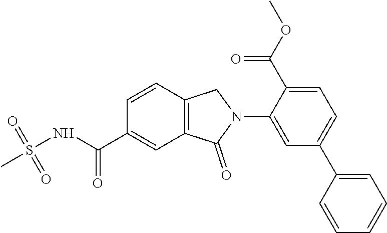 [SMILES: COC(=O)c1ccc(-c2ccccc2)cc1N1Cc2ccc(C(=O)NS(C)(=O)=O)cc2C1=O] |
| 104 | 3-(6-Methanesulfonylaminocarbonyl- 1-oxo-1,3-dihydroisoindol-2- yl)biphenyl-4-carboxylic acid | 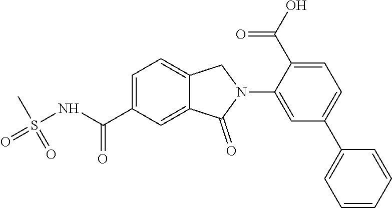 [SMILES: CS(=O)(=O)NC(=O)c1ccc2c(c1)C(=O)N(c1cc(-c3ccccc3)ccc1C(=O)O)C2] |
| 105 | 3′,4′-Difluoro-3-(6- methanesulfonylaminocarbonyl-1-oxo- 1,3-dihydroisoindol-2-yl)biphenyl-4- carboxylic acid methyl ester | 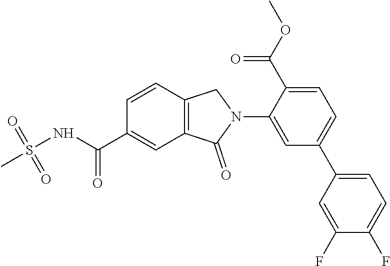 [SMILES: COC(=O)c1ccc(-c2ccc(F)c(F)c2)cc1N1Cc2ccc(C(=O)NS(C)(=O)=O)cc2C1=O] |
| 106 | 3′,4′-Difluoro-3-(6- methanesulfonylaminocarbonyl-1-oxo- 1,3-dihydroisoindol-2-yl)biphenyl-4- carboxylic acid | 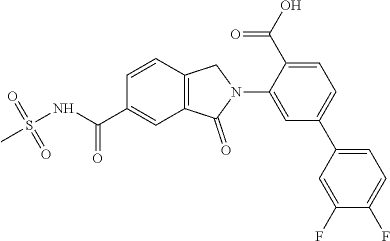 [SMILES: CS(=O)(=O)NC(=O)c1ccc2c(c1)C(=O)N(c1cc(-c3ccc(F)c(F)c3)ccc1C(=O)O)C2] |
| 107 | 3′,4′-Difluoro-3-(6- methanesulfonylaminocarbonyl-1-oxo- 1,3-dihydroisoindol-2-yl)biphenyl-4- carboxylic acid amide | 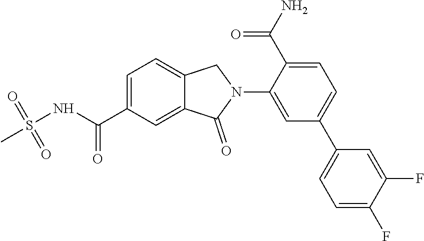 [SMILES: CS(=O)(=O)NC(=O)c1ccc2c(c1)C(=O)N(c1cc(-c3ccc(F)c(F)c3)ccc1C(N)=O)C2] |
| 108 | 3′,4′-Difluoro-3-(6- methanesulfonylaminocarbonyl-1-oxo- 1,3-dihydroisoindol-2-yl)biphenyl-4- carboxylic acid 2-dimethylamino-ethyl ester | 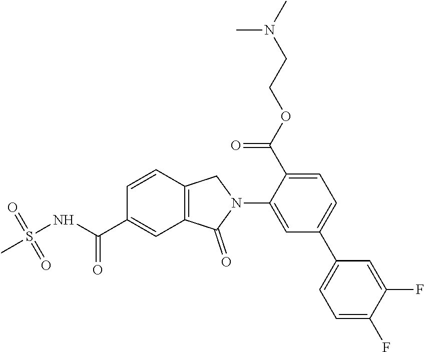 [SMILES: CN(C)CCOC(=O)c1ccc(-c2ccc(F)c(F)c2)cc1N1Cc2ccc(C(=O)NS(C)(=O)=O)cc2C1=O] |
| 109 | 3′,4′-Difluoro-3-(6- methanesulfonylaminocarbonyl-1-oxo- 1,3-dihydroisoindol-2-yl)biphenyl-4- carboxylic acid 2-pyrrolidin-1-yl-ethyl ester | 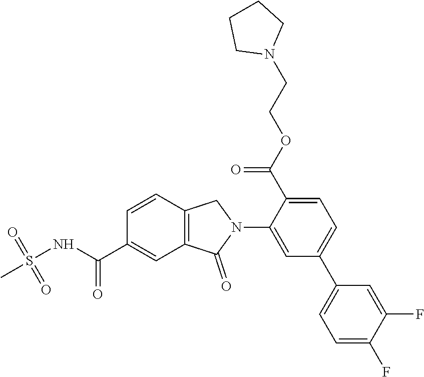 [SMILES: CS(=O)(=O)NC(=O)c1ccc2c(c1)C(=O)N(c1cc(-c3ccc(F)c(F)c3)ccc1C(=O)OCCN1CCCC1)C2] |
| 110 | 3′,4′-Difluoro-3-[1-oxo-6-(1H- [1,2,3]triazol-4-yl)-1,3-dihydroisoindol- 2-yl]biphenyl-4-carboxylic acid methyl ester | 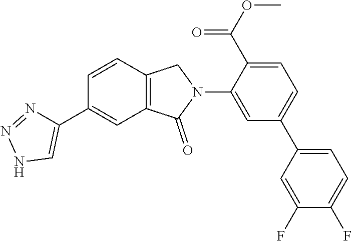 [SMILES: COC(=O)c1ccc(-c2ccc(F)c(F)c2)cc1N1Cc2ccc(-c3c[nH]nn3)cc2C1=O] |
| 111 | 3′,4′-Difluoro-3-[1-oxo-6-(1H- [1,2,3]triazol-4-yl)-1,3-dihydroisoindol- 2-yl]biphenyl-4-carboxylic acid | 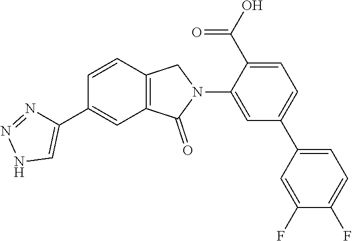 [SMILES: O=C(O)c1ccc(-c2ccc(F)c(F)c2)cc1N1Cc2ccc(-c3c[nH]nn3)cc2C1=O] |
| 112 | 3′,4′-Difluoro-3-[1-oxo-6-(1H- [1,2,3]triazol-4-yl)-1,3-dihydroisoindol- 2-yl]biphenyl-4-carboxylic acid 2- dimethylamino-ethyl ester | 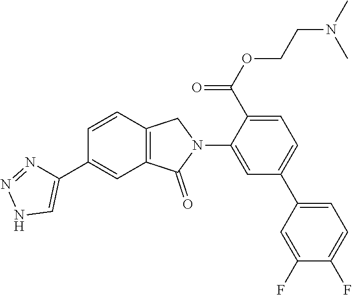 [SMILES: CN(C)CCOC(=O)c1ccc(-c2ccc(F)c(F)c2)cc1N1Cc2ccc(-c3c[nH]nn3)cc2C1=O] |
| 112a | 3′,4′-Difluoro-3-[1-oxo-6-(1H- [1,2,3]triazol-4-yl)-1,3-dihydroisoindol- 2-yl]biphenyl-4-carboxylic acid (2-oxo- 1,3-oxazolidin-5-yl)methyl ester | 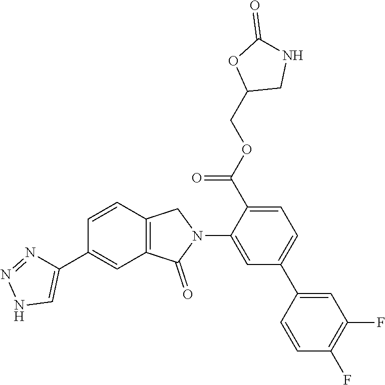 [SMILES: O=C1NCC(COC(=O)c2ccc(-c3ccc(F)c(F)c3)cc2N2Cc3ccc(-c4c[nH]nn4)cc3C2=O)O1] |
| 112b | 3′,4′-Difluoro-3-[1-oxo-6-(1H- [1,2,3]triazol-4-yl)-1,3-dihydroisoindol- 2-yl]biphenyl-4-carboxylic acid 2,3- dihydroxypropyl ester | 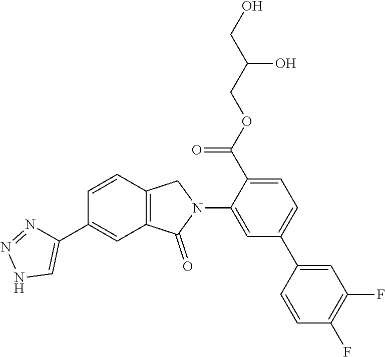 [SMILES: O=C(OCC(O)CO)c1ccc(-c2ccc(F)c(F)c2)cc1N1Cc2ccc(-c3c[nH]nn3)cc2C1=O] |
| 112c | 3′,4′-Difluoro-3-[1-oxo-6-(1H- [1,2,3]triazol-4-yl)-1,3-dihydroisoindol- 2-yl]biphenyl-4-carboxylic acid butan- 2-yl ester | 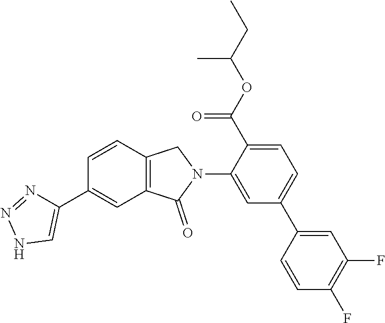 [SMILES: CCC(C)OC(=O)c1ccc(-c2ccc(F)c(F)c2)cc1N1Cc2ccc(-c3c[nH]nn3)cc2C1=O] |
| 112d | 3′,4′-Difluoro-3-[1-oxo-6-(1H- [1,2,3]triazol-4-yl)-1,3-dihydroisoindol- 2-yl]biphenyl-4-carboxylic acid 1- (dimethylamino)propan-2-yl ester | 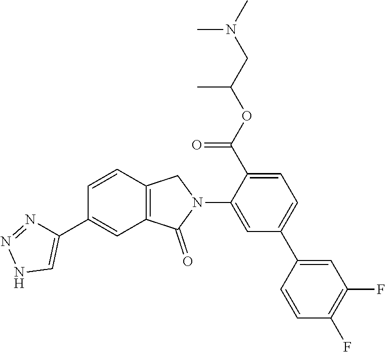 [SMILES: CC(CN(C)C)OC(=O)c1ccc(-c2ccc(F)c(F)c2)cc1N1Cc2ccc(-c3c[nH]nn3)cc2C1=O] |
| 113 | 3′,4′-Difluoro-3-[1-oxo-6-(1H- [1,2,3]triazol-4-yl)-1,3-dihydroisoindol- 2-yl]biphenyl-4-carboxylic acid amide | 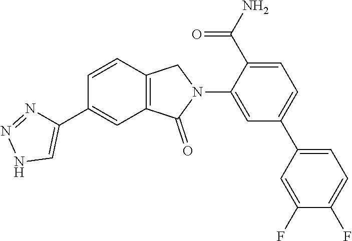 [SMILES: NC(=O)c1ccc(-c2ccc(F)c(F)c2)cc1N1Cc2ccc(-c3c[nH]nn3)cc2C1=O] |
| 114 | 3′,4′-Difluoro-3-[1-oxo-6-(1H- [1,2,3]triazol-4-yl)-1,3-dihydroisoindol- 2-yl]biphenyl-4-carboxylic acid 2- methoxy-ethyl ester | 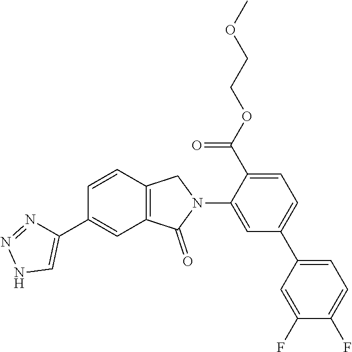 [SMILES: COCCOC(=O)c1ccc(-c2ccc(F)c(F)c2)cc1N1Cc2ccc(-c3c[nH]nn3)cc2C1=O] |
| 115 | 3′,4′-Difluoro-3-[1-oxo-6-(1H- [1,2,3]triazol-4-yl)-1,3-dihydroisoindol- 2-yl]biphenyl-4-carboxylic acid 2- pyrrolidin-1-yl-ethyl ester | 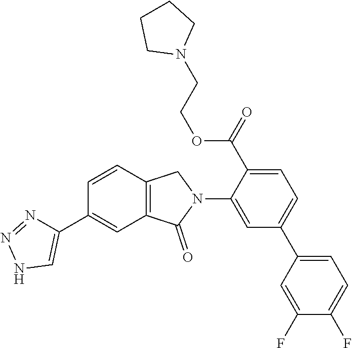 [SMILES: O=C(OCCN1CCCC1)c1ccc(-c2ccc(F)c(F)c2)cc1N1Cc2ccc(-c3c[nH]nn3)cc2C1=O] |
| 116 | 3′,4′-Difluoro-3-[1-oxo-6-(1H- [1,2,3]triazol-4-yl)-1,3-dihydro- isoindol-2-yl]-biphenyl-4-carboxylic acid (5-methyl-2-oxo-[1,3]dioxol-4- yl)methyl ester | 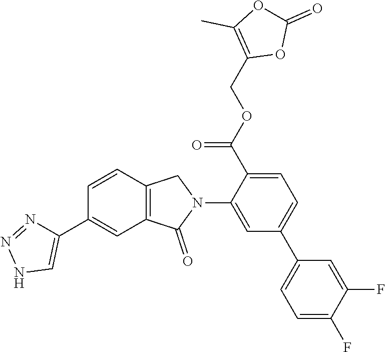 [SMILES: Cc1oc(=O)oc1COC(=O)c1ccc(-c2ccc(F)c(F)c2)cc1N1Cc2ccc(-c3c[nH]nn3)cc2C1=O] |
| 117 | 3′,4′-Difluoro-3-[1-oxo-6-(1H- [1,2,3]triazol-4-yl)-1,3-dihydro- isoindol-2-yl]biphenyl-4-carboxylic acid tert-butyl ester | 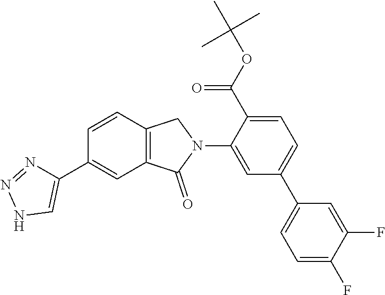 [SMILES: CC(C)(C)OC(=O)c1ccc(-c2ccc(F)c(F)c2)cc1N1Cc2ccc(-c3c[nH]nn3)cc2C1=O] |
| 118 | 3′,4′-Difluoro-3-[1-oxo-6-(1H- [1,2,3]triazol-4-yl)-1,3-dihydro- isoindol-2-yl]-biphenyl-2-carboxylic acid isopropyl ester | 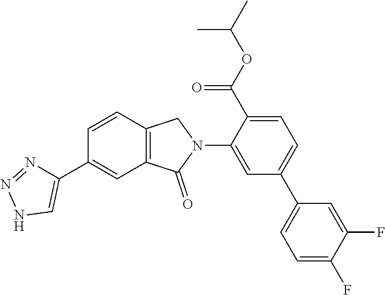 [SMILES: CC(C)OC(=O)c1ccc(-c2ccc(F)c(F)c2)cc1N1Cc2ccc(-c3c[nH]nn3)cc2C1=O] |
| 119 | 2-(4-Carbamoyl-3′,4′-difluorobiphenyl- 3-yl)-3-oxo-2,3-dihydro-1H-isoindole- 5-carboxylic acid | 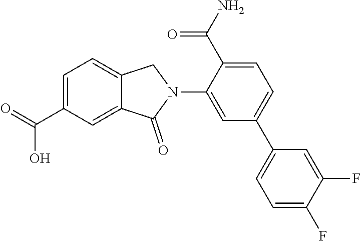 [SMILES: NC(=O)c1ccc(-c2ccc(F)c(F)c2)cc1N1Cc2ccc(C(=O)O)cc2C1=O] |
| 120 | 2-[4-(2-Dimethylamino- ethoxycarbonyl)-3′,4′-difluorobiphenyl- 3-yl]-3-oxo-2,3-dihydro-1H-isoindole- 5-carboxylic acid | 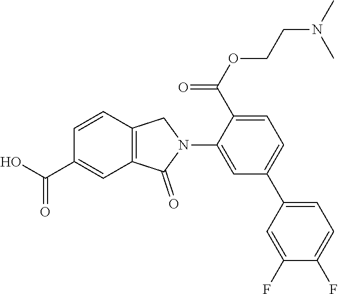 [SMILES: CN(C)CCOC(=O)c1ccc(-c2ccc(F)c(F)c2)cc1N1Cc2ccc(C(=O)O)cc2C1=O] |
| 121 | 3′,4′-Difluoro-3-(1-oxo-6-tetrazol-1-yl- 1,3-dihydroisoindol-2-yl)biphenyl-4- carboxylic acid methyl ester | 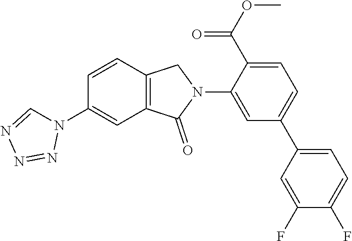 [SMILES: COC(=O)c1ccc(-c2ccc(F)c(F)c2)cc1N1Cc2ccc(-n3cnnn3)cc2C1=O] |
| 122 | 3′,4′-Difluoro-3-(1-oxo-6-tetrazol-1-yl- 1,3-dihydroisoindol-2-yl)biphenyl-4- carboxylic acid | 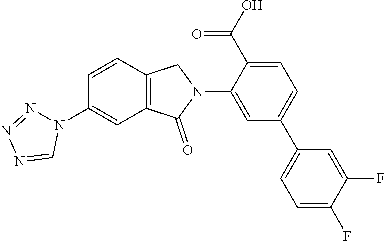 [SMILES: O=C(O)c1ccc(-c2ccc(F)c(F)c2)cc1N1Cc2ccc(-n3cnnn3)cc2C1=O] |
| 123 | 2-(4-Carbamoyl-3′,4′-difluorobiphenyl- 3-yl)-3-oxo-2,3-dihydro-1H-isoindole- 5-carboxylic acid butyl ester | 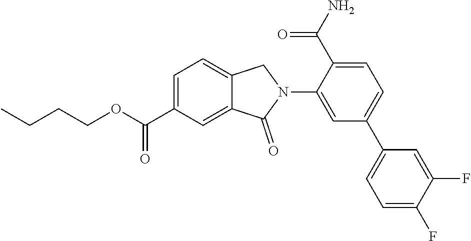 [SMILES: CCCCOC(=O)c1ccc2c(c1)C(=O)N(c1cc(-c3ccc(F)c(F)c3)ccc1C(N)=O)C2] |
| 124 | 3′,4′-Difluoro-3-(6- methanesulfonylaminocarbonyl-1-oxo- 1,3-dihydro-isoindol-2-yl)-biphenyl-4- carboxylic acid 3-dimethylamino- propyl ester | 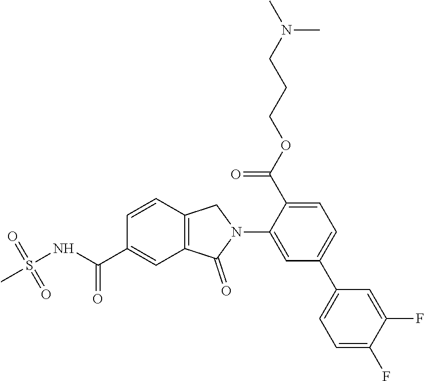 [SMILES: CN(C)CCCOC(=O)c1ccc(-c2ccc(F)c(F)c2)cc1N1Cc2ccc(C(=O)NS(C)(=O)=O)cc2C1=O] |
| 125 | 3′,4′-Difluoro-3-(6- methanesulfonylaminocarbonyl-1-oxo- 1,3-dihydro-isoindol-2-yl)-biphenyl-4- carboxylic acid isopropyl ester | 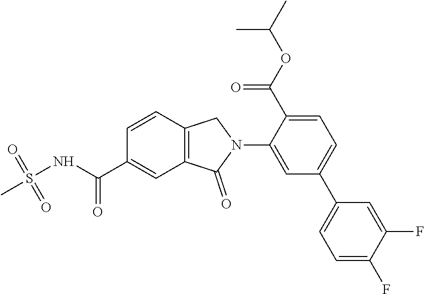 [SMILES: CC(C)OC(=O)c1ccc(-c2ccc(F)c(F)c2)cc1N1Cc2ccc(C(=O)NS(C)(=O)=O)cc2C1=O] |
| 126 | 2-(4-Carboxy-3′,4′-difluoro-biphenyl-3- yl)-3-oxo-2,3-dihydro-1H-isoindole-5- carboxylic acid | 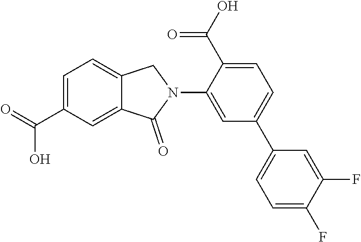 [SMILES: O=C(O)c1ccc2c(c1)C(=O)N(c1cc(-c3ccc(F)c(F)c3)ccc1C(=O)O)C2] |
| 127 | 2-(3′,4′-Difluoro-4- methoxycarbonylbiphenyl-3-yl)-3-oxo- 2,3-dihydro-1H-isoindole-5-carboxylic acid methyl ester | 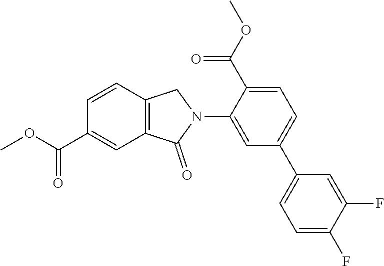 [SMILES: COC(=O)c1ccc2c(c1)C(=O)N(c1cc(-c3ccc(F)c(F)c3)ccc1C(=O)OC)C2] |
| 128 | 2-(4-Carboxy-3′,4′-difluoro-biphenyl-3- yl)-3-oxo-2,3-dihydro-1H-isoindole-5- carboxylic acid butyl ester | 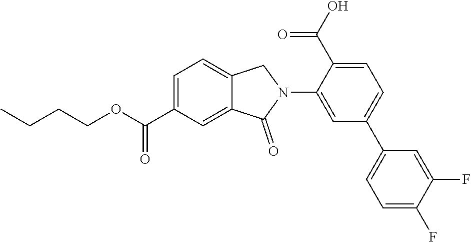 [SMILES: CCCCOC(=O)c1ccc2c(c1)C(=O)N(c1cc(-c3ccc(F)c(F)c3)ccc1C(=O)O)C2] |
| 129 | 2-(4-Carboxy-3′,4′-difluorobiphenyl-3- yl)-3-oxo-2,3-dihydro-1H-isoindole-5- carboxylic acid 2-dimethylamino-ethyl ester | 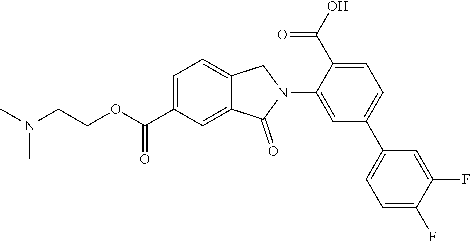 [SMILES: CN(C)CCOC(=O)c1ccc2c(c1)C(=O)N(c1cc(-c3ccc(F)c(F)c3)ccc1C(=O)O)C2] |
| 130 | 3-(6-Acetylamino-1-oxo-1,3- dihydroisoindol-2-yl)-3′,4′-difluoro- biphenyl-4-carboxylic acid | 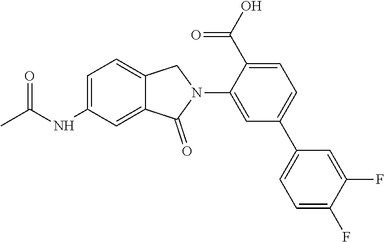 [SMILES: CC(=O)Nc1ccc2c(c1)C(=O)N(c1cc(-c3ccc(F)c(F)c3)ccc1C(=O)O)C2] |
| 131 | 3′,4′-Difluoro-3-(6- methanesulfonylamino-1-oxo-1,3- dihydro-isoindole-2-yl)-biphenyl-4- carboxylic acid | 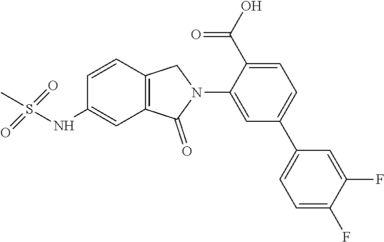 [SMILES: CS(=O)(=O)Nc1ccc2c(c1)C(=O)N(c1cc(-c3ccc(F)c(F)c3)ccc1C(=O)O)C2] |
| 132 | 3′,4′-Difluoro-3-[1-oxo-6-(toluene-4- sulfonylamino)-1,3-dihydro-isoindol-2- yl]-biphenyl-4-carboxylic acid | 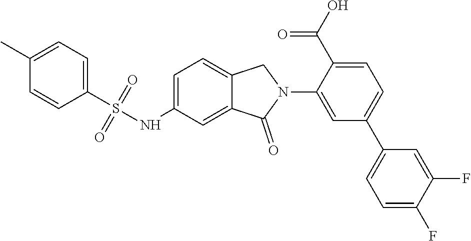 [SMILES: Cc1ccc(S(=O)(=O)Nc2ccc3c(c2)C(=O)N(c2cc(-c4ccc(F)c(F)c4)ccc2C(=O)O)C3)cc1] |
| 133 | 2-(3-Cyano-4,5-diphenylthiophen-2- yl)-1,3-dioxo-2,3-dihydro-1H- isoindole-5-carboxylic acid | 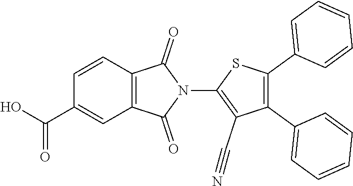 [SMILES: N#Cc1c(N2C(=O)c3ccc(C(=O)O)cc3C2=O)sc(-c2ccccc2)c1-c1ccccc1] |
| 134 | 2-(3-Carboxybiphenyl-4-yl)-1,3-dioxo- 2,3-dihydro-1H-isoindole-5-carboxylic acid | 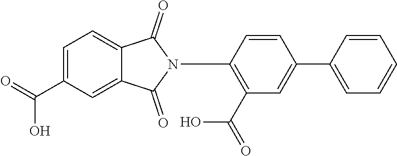 [SMILES: O=C(O)c1ccc2c(c1)C(=O)N(c1ccc(-c3ccccc3)cc1C(=O)O)C2=O] |
| 135 | N-(Benzenesulfonyl)-2-(3-(1H- tetrazol-5-yl)-[1,1′-biphenyl]-4-yl)-1,3- dioxo-2,3-dihydro-1H-isoindole-5- carboxamide | 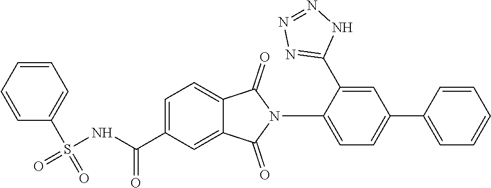 [SMILES: O=C(NS(=O)(=O)c1ccccc1)c1ccc2c(c1)C(=O)N(c1ccc(-c3ccccc3)cc1-c1nnn[nH]1)C2=O] |
| 136 | (2S)-2-({2-[3-Cyano-4-(4- methoxypehnyl)-5-methyl-thiophen-2- yl]-1,3-dioxo-2,3-dihydro-1H- isoindole-5-carbonyl}amino)-3-(4- hydroxyphenyl)propanoic acid | 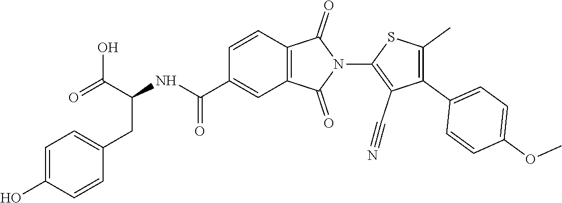 [SMILES: COc1ccc(-c2c(C)sc(N3C(=O)c4ccc(C(=O)N[C@@H](Cc5ccc(O)cc5)C(=O)O)cc4C3=O)c2C#N)cc1] |
| 137 | (2S)-2-({2-[3-Cyano-4-(4- methoxyphenyl)-5-methyl-thiophen-2- yl]-1,3-dioxo-2,3-dihydro-1H- isoindole-5-carbonyl}-amino)-3- phenylpropanoic acid | 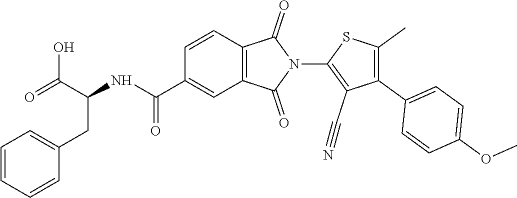 [SMILES: COc1ccc(-c2c(C)sc(N3C(=O)c4ccc(C(=O)N[C@@H](Cc5ccccc5)C(=O)O)cc4C3=O)c2C#N)cc1] |
| 138 | 2-(4-Carboxy-3′,4′-difluoro[1,1′- biphenyl]-3-yl)-1,3-dioxo-2,3-dihydro- 1H-isoindole-5-carboxylic acid | 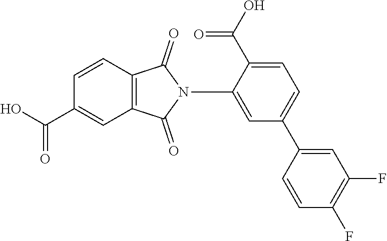 [SMILES: O=C(O)c1ccc2c(c1)C(=O)N(c1cc(-c3ccc(F)c(F)c3)ccc1C(=O)O)C2=O] |
| 139 | 2-(4-Carboxy-2′,3′,4′-trifluoro[1,1′- biphenyl]-3-yl)-1,3-dioxo-2,3-dihydro- 1H-isoindole-5-carboxylic acid | 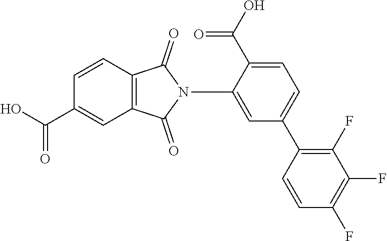 [SMILES: O=C(O)c1ccc2c(c1)C(=O)N(c1cc(-c3ccc(F)c(F)c3F)ccc1C(=O)O)C2=O] |
| 140 | 2-(4-Carboxy-2′,4′,5′-trifluoro[1,1′- biphenyl]-3-yl)-1,3-dioxo-2,3-dihydro- 1H-isoindole-5-carboxylc acid | 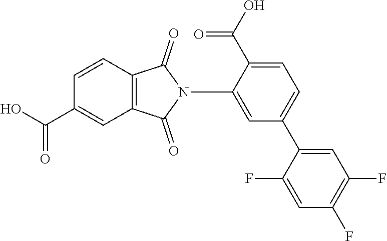 [SMILES: O=C(O)c1ccc2c(c1)C(=O)N(c1cc(-c3cc(F)c(F)cc3F)ccc1C(=O)O)C2=O] |
| 141 | 2-(4-Carboxy-4′-methyl[1,1′-biphenyl]- 3-yl)-1,3-dioxo-2,3-dihydro-1H- isoindole-5-carboxylic acid | 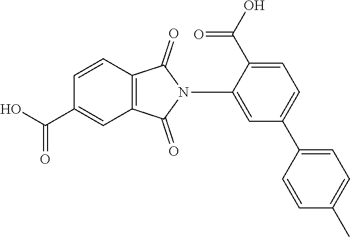 [SMILES: Cc1ccc(-c2ccc(C(=O)O)c(N3C(=O)c4ccc(C(=O)O)cc4C3=O)c2)cc1] |
| 142 | 2-(4-Carboxy-2′,4′-dichloro[1,1′- biphenyl]-3-yl)-1,3-dioxo-2,3-dihydro- 1H-isoindole-5-carboxylic acid | 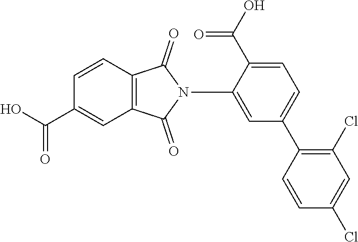 [SMILES: O=C(O)c1ccc2c(c1)C(=O)N(c1cc(-c3ccc(Cl)cc3Cl)ccc1C(=O)O)C2=O] |
| 143 | 2-(4-Carboxy-4′-chloro-3′-fluoro[1,1′- biphenyl]-3-yl)-1,3-dioxo-2,3-dihydro- 1H-isoindole-5-carboxylic acid | 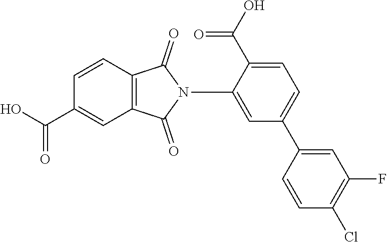 [SMILES: O=C(O)c1ccc2c(c1)C(=O)N(c1cc(-c3ccc(Cl)c(F)c3)ccc1C(=O)O)C2=O] |
| 144 | 2-(4-Carboxy-3′-fluoro-4′- methoxy[1,1′-biphenyl]-3-yl)-1,3- dioxo-2,3-dihydro-1H-isoindole-5- carboxylic acid | 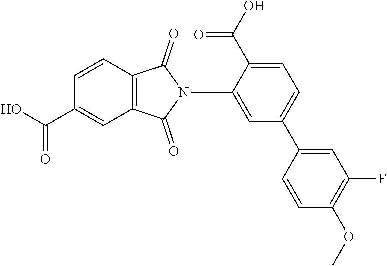 [SMILES: COc1ccc(-c2ccc(C(=O)O)c(N3C(=O)c4ccc(C(=O)O)cc4C3=O)c2)cc1F] |
| 144a | 2-(4-carboxy-3′,4′-difluoro[1,1′- biphenyl]-3-yl)-6-hydroxy-1,3-dioxo- 2,3-dihydro-1H-isoindole-5-carboxylic acid | 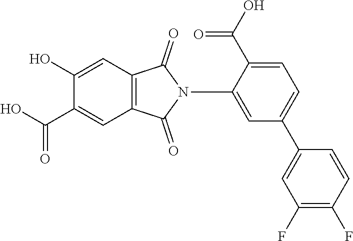 [SMILES: O=C(O)c1cc2c(cc1O)C(=O)N(c1cc(-c3ccc(F)c(F)c3)ccc1C(=O)O)C2=O] |
| 144b | 3-[5-chloro-1,3-dioxo-6-(H-1,2,3- triazol-5-yl)-2,3-dihydro-1H-isoindol-2- yl]-3′,4′-difluoro[1,1′-biphenyl]-4- carboxylic acid | 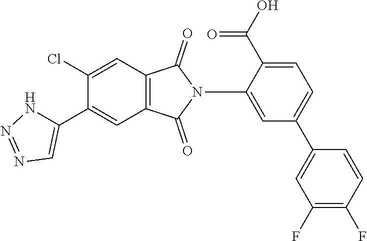 [SMILES: O=C(O)c1ccc(-c2ccc(F)c(F)c2)cc1N1C(=O)c2cc(Cl)c(-c3cnn[nH]3)cc2C1=O] |
| 144c | 3-[5-chloro-1,3-dioxo-6-(1H-1,2,3- triazol-5-yl)-2,3-dihydro-1H-isoindol-2- yl][1,1′-biphenyl]-4-carboxylic acid | 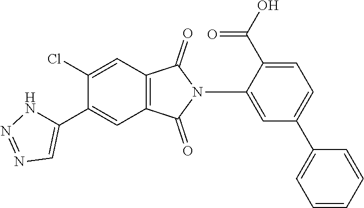 [SMILES: O=C(O)c1ccc(-c2ccccc2)cc1N1C(=O)c2cc(Cl)c(-c3cnn[nH]3)cc2C1=O] |
| 145 | 2-(3-cyanobiphenyl-4-yl)-1,3- dioxoisoindoline-5-carboxylic acid | 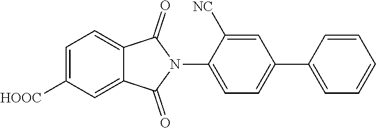 [SMILES: N#Cc1cc(-c2ccccc2)ccc1N1C(=O)c2ccc(C(=O)O)cc2C1=O] |
| 146 | 2-(4-(4-fluorophenyl)-5-phenylpyridin- 2-yl)-5-(1H-1,2,3-triazol-4- yl)isoindoline-1,3-dione | 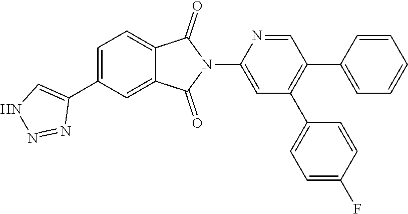 [SMILES: O=C1c2ccc(-c3c[nH]nn3)cc2C(=O)N1c1cc(-c2ccc(F)cc2)c(-c2ccccc2)cn1] |
| 147 | 2-(1-methyl-2-oxo-5-phenyl-1,2- dihydropyridin-3-yl)-5-(1H-1,2,3- triazol-4-yl)isoindoline-1,3-dione | 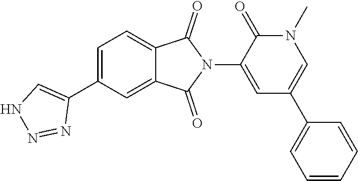 [SMILES: Cn1cc(-c2ccccc2)cc(N2C(=O)c3ccc(-c4c[nH]nn4)cc3C2=O)c1=O] |
| 148 | 3-(1,3-dioxo-5-(1H-1,2,3-triazol-4- yl)isoindolin-2-yl)biphenyl-4- sulfonamide | 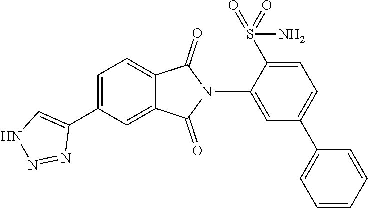 [SMILES: NS(=O)(=O)c1ccc(-c2ccccc2)cc1N1C(=O)c2ccc(-c3c[nH]nn3)cc2C1=O] |
| 149 | 2-(2-(3-cyano-4-(4-methoxyphenyl)-5- methylthiophen-2-yl)-1,3- dioxoisoindoline-5- carboxamido)acetic acid | 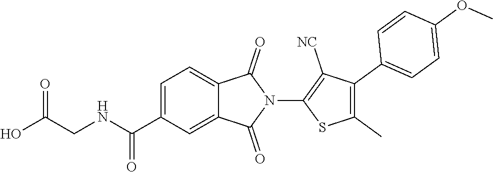 [SMILES: COc1ccc(-c2c(C)sc(N3C(=O)c4ccc(C(=O)NCC(=O)O)cc4C3=O)c2C#N)cc1] |
| 150 | methyl 2-(4-hydroxybiphenyl-3-yl)-1,3- dioxoisoindoline-5-carboxylate | 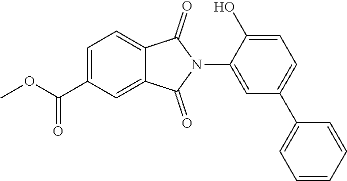 [SMILES: COC(=O)c1ccc2c(c1)C(=O)N(c1cc(-c3ccccc3)ccc1O)C2=O] |
| 151 | 2-(4-methylpiperazin-1-yl)ethyl 2-(3- cyano-4-(4-methoxyphenyl)-5- methylthiophen-2-yl)-1,3- dioxoisoindoline-5-carboxylate | 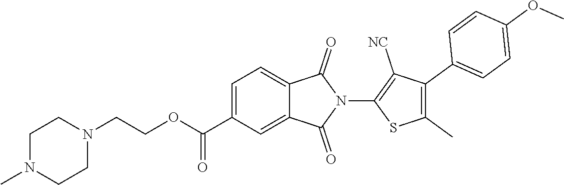 |
| 152 | methyl 4-(1,3-dioxo-5- (phenylsulfonylcarbamoyl)isoindolin- 2-yl)biphenyl-3-carboxylate | 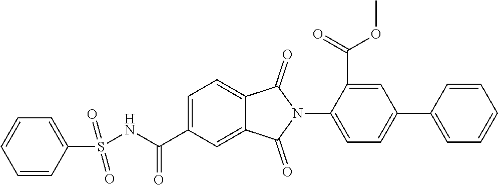 [SMILES: COC(=O)c1cc(-c2ccccc2)ccc1N1C(=O)c2ccc(C(=O)NS(=O)(=O)c3ccccc3)cc2C1=O] |
| 153 | 2-aminoethyl 3-(1,3-dioxo-5-(1H- 1,2,3-triazol-4-yl)isoindolin-2- yl)biphenyl-4-carboxylate | 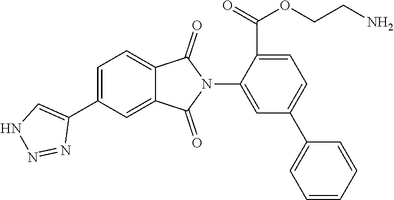 [SMILES: NCCOC(=O)c1ccc(-c2ccccc2)cc1N1C(=O)c2ccc(-c3c[nH]nn3)cc2C1=O] |
| 154 | 2-(2-(1H-tetrazol-5-yl)-5- methoxyphenyl)-5-(1H-1,2,3-triazol-4- yl)isoindoline-1,3-dione | 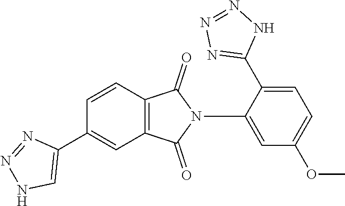 [SMILES: COc1ccc(-c2nnn[nH]2)c(N2C(=O)c3ccc(-c4c[nH]nn4)cc3C2=O)c1] |
| 155 | 2-(4-methylpiperazin-1-yl)ethyl 3′,4′- difluoro-3-(6-(hydroxymethyl)-1- oxoisoindolin-2-yl)biphenyl-4- carboxylate | 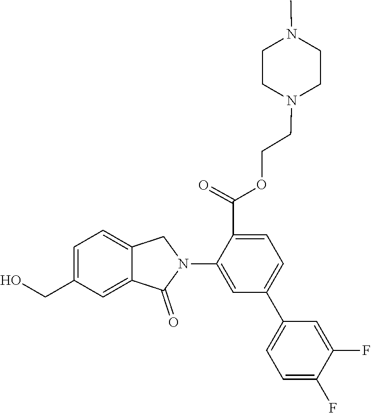 [SMILES: CN1CCN(CCOC(=O)c2ccc(-c3ccc(F)c(F)c3)cc2N2Cc3ccc(CO)cc3C2=O)CC1] |
| 156 | isopropyl 3′,4′-difluoro-3-(6- (hydroxymethyl)-1-oxoisoindolin-2- yl)biphenyl-4-carboxylate | 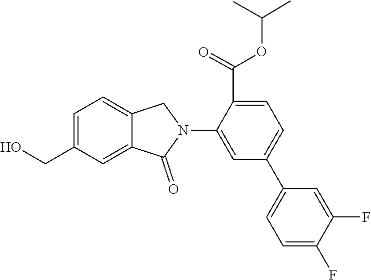 [SMILES: CC(C)OC(=O)c1ccc(-c2ccc(F)c(F)c2)cc1N1Cc2ccc(CO)cc2C1=O] |
| 157 | methyl 3′,4′-difluoro-3-(6- (hydroxymethyl)-1-oxoisoindolin-2- yl)biphenyl-4-carboxylate | 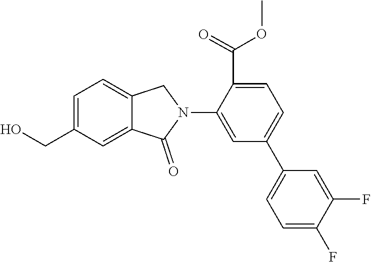 [SMILES: COC(=O)c1ccc(-c2ccc(F)c(F)c2)cc1N1Cc2ccc(CO)cc2C1=O] |
| 158 | 3′,4′-difluoro-3-(6-(hydroxymethyl)-1- oxoisoindolin-2-yl)biphenyl-4- carboxylic acid | 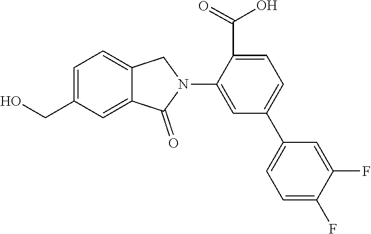 [SMILES: O=C(O)c1ccc(-c2ccc(F)c(F)c2)cc1N1Cc2ccc(CO)cc2C1=O] |
| 159 | ethyl 3′,4′-difluoro-3-(6- (hydroxymethyl)-1-oxoisoindolin-2- yl)biphenyl-4-carboxylate | 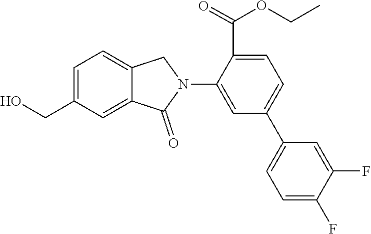 [SMILES: CCOC(=O)c1ccc(-c2ccc(F)c(F)c2)cc1N1Cc2ccc(CO)cc2C1=O] |
| 160 | 3-(6-carbamoyl-1-oxoisoindolin-2-yl)- 3′,4′-difluorobiphenyl-4-carboxylic acid | 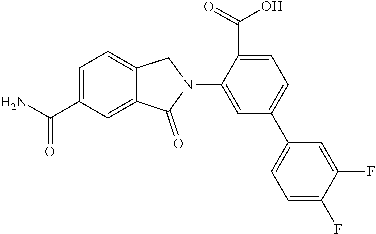 [SMILES: NC(=O)c1ccc2c(c1)C(=O)N(c1cc(-c3ccc(F)c(F)c3)ccc1C(=O)O)C2] |
| 161 | 3′,4′-difluoro-3-(6-(5-(hydroxymethyl)- 1H-1,2,3-triazol-4-yl)-1-oxoisoindolin- 2-yl)biphenyl-4-carboxylic acid | 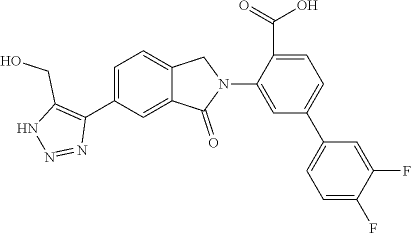 [SMILES: O=C(O)c1ccc(-c2ccc(F)c(F)c2)cc1N1Cc2ccc(-c3nn[nH]c3CO)cc2C1=O] |
| 162 | 3-(6-(5-benzoyl-1H-1,2,3-triazol-4-yl)- 1-oxoisoindolin-2-yl)-3′,4′- difluorobiphenyl-4-carboxylic acid | 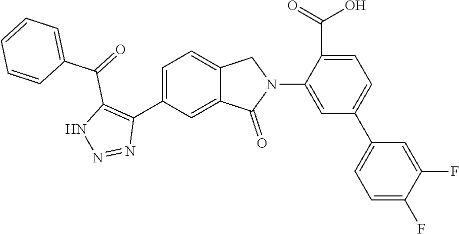 [SMILES: O=C(O)c1ccc(-c2ccc(F)c(F)c2)cc1N1Cc2ccc(-c3nn[nH]c3C(=O)c3ccccc3)cc2C1=O] |
| 163 | 3′,4′-difluoro-3-(1-oxo-6-(5-phenyl-1H- 1,2,3-triazol-4-yl)isoindolin-2- yl)biphenyl-4-carboxylic acid | 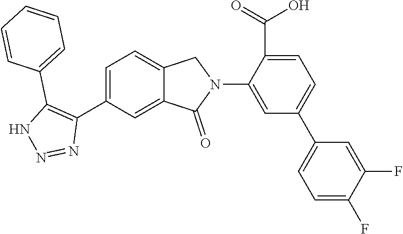 [SMILES: O=C(O)c1ccc(-c2ccc(F)c(F)c2)cc1N1Cc2ccc(-c3nn[nH]c3-c3ccccc3)cc2C1=O] |
| 164 | 3-(6-(5-carbamoyl-1H-1,2,3-triazol-4- yl)-1-oxoisoindolin-2-yl)-3′,4′- difluorobiphenyl-4-carboxylic acid | 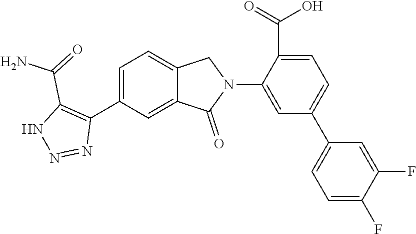 [SMILES: NC(=O)c1[nH]nnc1-c1ccc2c(c1)C(=O)N(c1cc(-c3ccc(F)c(F)c3)ccc1C(=O)O)C2] |
| 165 | 3-(6-(5-(dimethylcarbamoyl)-1H-1,2,3- triazol-4-yl)-1-oxoisoindolin-2-yl)-3′,4′- difluorobiphenyl-4-carboxylic acid | 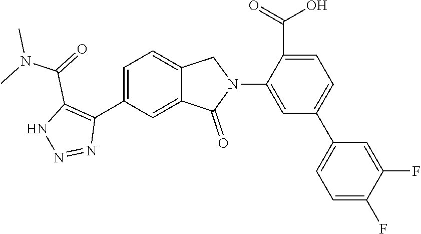 [SMILES: CN(C)C(=O)c1[nH]nnc1-c1ccc2c(c1)C(=O)N(c1cc(-c3ccc(F)c(F)c3)ccc1C(=O)O)C2] |
| 166 | ethyl 3′,4′-difluoro-3-[1-oxo-6-(1H- 1,2,3-triazol-5-yl)-2,3-dihydro-1H- isoindol-2-yl][1,1′-biphenyl]-4- carboxylate | 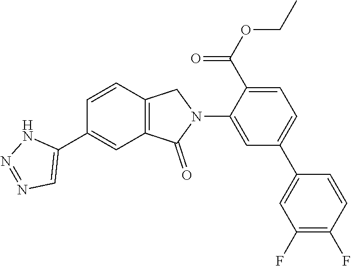 [SMILES: CCOC(=O)c1ccc(-c2ccc(F)c(F)c2)cc1N1Cc2ccc(-c3cnn[nH]3)cc2C1=O] |
| 167 | ethyl 3-[1,3-dioxo-5-(1H-1,2,3-triazol- 5-yl)-1,3-dihydro-2H-isoindol-2-yl]- 3′,4′-difluoro[1,1′-biphenyl]-4- carboxylate | 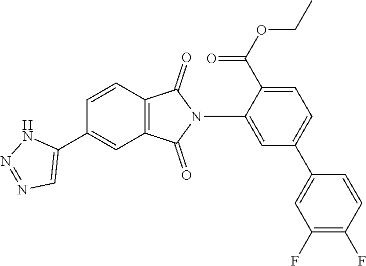 [SMILES: CCOC(=O)c1ccc(-c2ccc(F)c(F)c2)cc1N1C(=O)c2ccc(-c3cnn[nH]3)cc2C1=O] |
| 168 | 2-(acetyloxy)ethyl 3-[1,3-dioxo-5-(1H- 1,2,3-triazol-5-yl)-2,3-dihydro-1H- isoindol-2-yl]-3′,4′-difluoro[1,1′- biphenyl]-4-carboxylate | 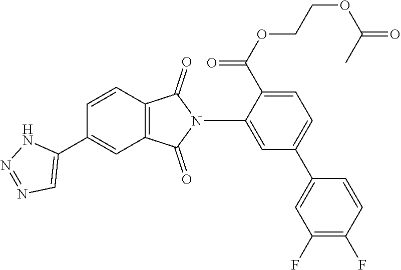 [SMILES: CC(=O)OCCOC(=O)c1ccc(-c2ccc(F)c(F)c2)cc1N1C(=O)c2ccc(-c3cnn[nH]3)cc2C1=O] |
| 169 | 6-bromo-2-(4-carboxy-3′,4′- difluoro[1,1′-biphenyl]-3-yl)-1,3-dioxo- 2,3-dihydro-1H-isoindole-5-carboxylic acid | 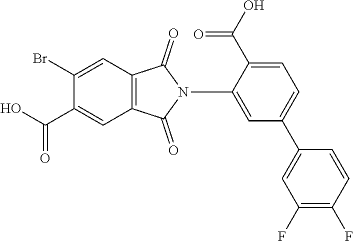 [SMILES: O=C(O)c1cc2c(cc1Br)C(=O)N(c1cc(-c3ccc(F)c(F)c3)ccc1C(=O)O)C2=O] |
| 170 | 2-(4-carboxy-3′,4′-difluoro[1,1′- biphenyl]-3-yl)-6-methoxy-1,3-dioxo- 2,3-dihydro-1H-isoindole-5-carboxylic acid | 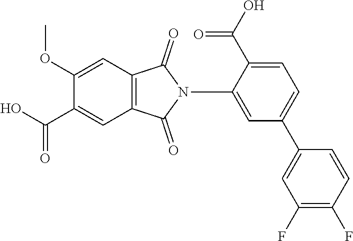 [SMILES: COc1cc2c(cc1C(=O)O)C(=O)N(c1cc(-c3ccc(F)c(F)c3)ccc1C(=O)O)C2=O] |

Also disclosed herein is a compound of Formula (VII):

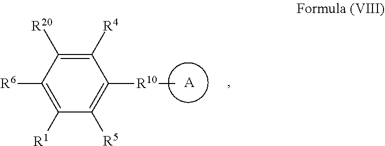

or a pharmaceutically acceptable salt thereof, wherein:

A is selected from:

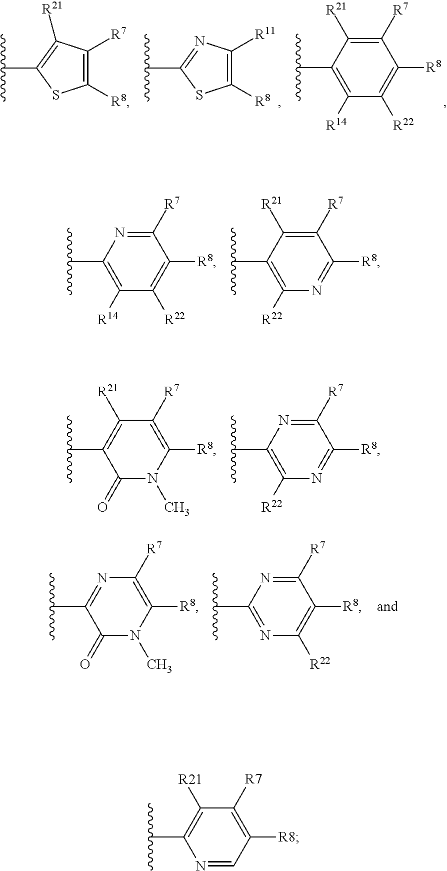

R¹ is selected from hydrogen, halogen, hydroxyl, C₁-C₆ alkyl, and C₁-C₆ alkoxy,

wherein the C₁-C₆ alkyl and C₁-C₆ alkoxy are optionally substituted with one or more halogens;

each R² and R³ is independently selected from hydrogen and C₁-C₆ alkyl,

wherein the C₁-C₆ alkyl is optionally substituted with one or more halogens;

or R² and R³ are taken together with the N to which they are attached to form a 3- to 10-membered heterocycle optionally substituted with one or more substituents independently selected from C₁-C₆ alkyl;

R⁴ is selected from hydrogen, halogen, C₁-C₆ alkyl, and C₁-C₆alkoxy,

wherein the C₁-C₆ alkyl and C₁-C₆ alkoxy are optionally substituted with one or more substituents independently selected from halogens;

R⁵ is selected from —C(═O)OR¹⁵, —C(═O)NR²R³, —S(═O)₂NR²R³, —C(═O)NHR¹⁵, —CH₂OH, 3-hydroxyoxetan-3-yl, and —NH₂;

R⁶ is selected from hydrogen, halogen, hydroxyl, 5-membered heteroaryl, C₁-C₆ alkyl, —C(═O)OR¹⁵, —C(═O)R¹², —C(═O)NHR¹⁵, and —C(═O)N═S(═X³)(CH₃)₂,

wherein the C₁-C₆ alkyl are optionally substituted with one or more R⁹, and

wherein 5-membered heteroaryl contains at least two heteroatoms and is optionally substituted with one or more substituents independently selected from R¹⁷;

R⁷ is selected from hydrogen, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,

wherein the C₁-C₆ alkyl and C₁-C₆ alkoxy are optionally substituted with one or more halogens, and

wherein C₃-C₈ cycloalkyl, —O—C₃-C₈ cycloalkyl, 3- to 10-membered heterocycloalkyl, —O—(3- to 10-membered heterocycloalkyl), aryl and heteroaryl are optionally substituted with one or more R²⁴;

R⁸ is selected from hydrogen, —NO₂, C₁-C₆ alkyl, aryl, and heteroaryl,

wherein the C₁-C₆ alkyl and C₁-C₆ alkoxy are optionally substituted with one or more substituents independently selected at each occurrence from halogen; and

wherein aryl and heteroaryl are optionally substituted with one or more substituents independently selected at each occurrence from R²³;

or R⁷ and R⁸ are taken together to form a C₅-C₁₀ carbocycle or 5- to 10-membered heterocycle,

wherein C₅-C₁₀ carbocycle and 5- to 10-membered heterocycle are optionally substituted with one or more substituents independently selected from halogen, hydroxyl, —NO₂, C₁-C₆ alkyl, C₁-C₆ alkoxy, C₃-C₈ cycloalkyl, —O—C₃-C₈cycloalkyl, 3- to 10-membered heterocycloalkyl, —O—(3- to 10-membered heterocycloalkyl), aryl, and heteroaryl,

wherein the C₁-C₆ alkyl and C₁-C₆ alkoxy are optionally substituted with one or more halogens, and

wherein aryl, heteroaryl, C₃-C₈ cycloalkyl, —O—C₃-C₈ cycloalkyl, 3- to 10-membered heterocycloalkyl, and μ-O-(3- to 10-membered heterocycloalkyl) are optionally substituted with one or more R²³;

each R⁹ is independently selected from hydroxy and —COOH;

R¹⁰ is selected from —C(═O)—X¹—, —CH₂—X¹—, —X¹—C(═O)—, and —X¹—CH₂—;

R¹¹ is selected from hydrogen, —NO₂, C₁-C₆ alkyl, C₁-C₆ alkoxy, C₃-C₈ cycloalkyl, —O—C₃-C₈ cycloalkyl, 3- to 10-membered heterocycloalkyl, and —O—(3- to 10-membered heterocycloalkyl),

wherein the C₁-C₆ alkyl and C₁-C₆ alkoxy are optionally substituted with one or more halogens, and

wherein C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;

R¹² is selected from alanine, arginine, asparagine, aspartic acid, asparagine, aspartic acid, cysteine, glutamic acid, glutamine, glutamine, glutamic acid, glycine, histidine, isoleucine, leucine, lysine, methionine, phenylalanine, proline, serine, threonine, tryptophan, tyrosine, and valine, wherein the point of attachment of R¹² is a nitrogen atom;

R¹⁴ is selected from hydrogen, halogen, hydroxyl, nitrile, —C(═O)CR¹⁵ and —C(═O)OR¹⁵;

each R¹⁵ is independently selected from hydrogen and C₁-C₆ alkyl, -heterocyclyl, wherein the C₁-C₆ alkyl is optionally substituted with one or more substituents independently selected at each occurrence from —C(═O)NR²R³, -heterocyclyl, —NR²R³;

wherein the heterocyclyl is optionally substituted with one or more substituents independently selected at each occurrence from R² and R³.

R¹⁷ is selected from C₁-C₆ alkyl, aryl, and 6-membered heteroaryl,

wherein the C₁-C₆ alkyl is optionally substituted with one or more hydroxy, and

wherein aryl and 6-membered heteroaryl are optionally substituted with one or more substituents independently selected from halogen, —R², and —OR²;

R²⁰ is selected from hydrogen, halogen, hydroxyl, —COOH, —NC(═O)R², —OR², 5-membered heteroaryl, C₁-C₆ alkyl, —C(═O)N═S(═X³)(CH₃)₂, —CH₂(OH)CH₂OH and —NH—SO₂—R²,

wherein the 5-membered heteroaryl contains at least two heteroatoms, and

wherein the C₁-C₆ alkyl is optionally substituted with one or more substituents independently selected from 5-membered heteroaryl, wherein the 5-membered heteroaryl contains at least two heteroatoms;

R²¹ is selected from hydrogen and nitrile;

R²² is selected from hydrogen and hydroxy;

each R²³ is independently selected from halogen, C₁-C₆ alkyl, C₁-C₆ alkoxy,

wherein the C₁-C₆ alkyl and C₁-C₆ alkoxy are optionally substituted with one or more substituents independently selected from halogens;

each R²⁴ is independently selected from halogen, C₁-C₆ alkyl, C₁-C₆ alkoxy, 5-membered heteroaryl wherein the C₁-C₆ alkyl and C₁-C₆ alkoxy are optionally substituted with one or more substituents independently selected from halogens;

each X¹ is independently selected from —NR²— and —CR²R³—; and

each X³ is independently selected from NH and 0.

In some embodiments, the compound disclosed herein has the structure provided in Table 6; in some embodiments the invention provides at least one of the compounds selected from the Table 6

TABLE 6

| | Structure | IUPAC |
|---|---|---|
| Example 86 | 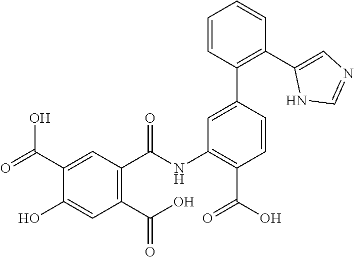 [SMILES: O=C(O)c1cc(C(=O)Nc2cc(-c3ccccc3-c3cnc[nH]3)ccc2C(=O)O)c(C(=O)O)cc1O] | 2-{[4-carboxy-2′-(1H-imidazol-4-yl)-[1,1′- biphenyl]-3-yl]carbamoyl}-5- hydroxybenzene-1,4-dicarboxylic acid |
| Example 85 | 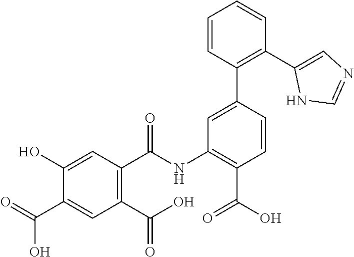 [SMILES: O=C(O)c1cc(C(=O)O)c(C(=O)Nc2cc(-c3ccccc3-c3cnc[nH]3)ccc2C(=O)O)cc1O] | 4-{[4-carboxy-2′-(1H-imidazol-4-yl)-[1,1′- biphenyl]-3-yl]carbamoyl}-6- hydroxybenzene-1,3-dicarboxylic acid |
| Example 84 | 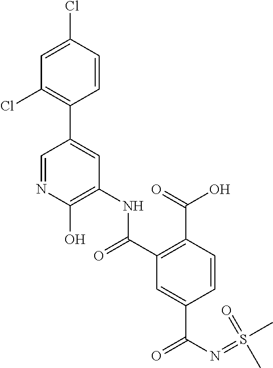 [SMILES: CS(C)(=O)=NC(=O)c1ccc(C(=O)O)c(C(=O)Nc2cc(-c3ccc(Cl)cc3Cl)cnc2O)c1] | 2-{[5-(2,4-dichlorophenyl)-2-oxo-1,2- dihydropyridin-3-yl]carbamoyl}-4- {[dimethyl(oxo)-λ⁶- sulfanylidene]carbamoyl}benzoic acid |
| Example 83 | 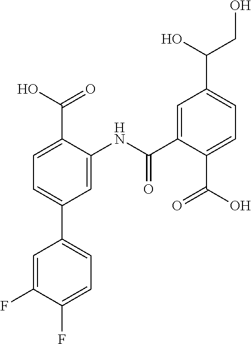 [SMILES: O=C(O)c1ccc(-c2ccc(F)c(F)c2)cc1NC(=O)c1cc(C(O)CO)ccc1C(=O)O] | 3-[2-carboxy-5-(1,2- dihydroxyethyl)benzamido]-3′,4′-difluoro- [1,1′-biphenyl]-4-carboxylic acid |
| Example 82 | 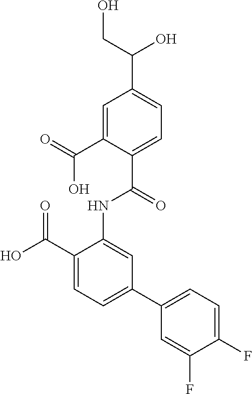 [SMILES: O=C(O)c1ccc(-c2ccc(F)c(F)c2)cc1NC(=O)c1ccc(C(O)CO)cc1C(=O)O] | 3-[2-carboxy-4-(1,2- dihydroxyethyl)benzamido]-3′,4′-difluoro- [1,1′-biphenyl]-4-carboxylic acid |
| Example 81 | 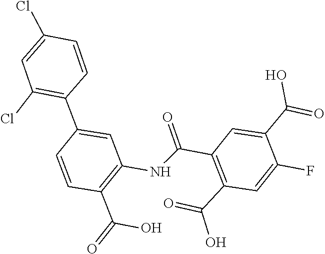 [SMILES: O=C(O)c1cc(C(=O)Nc2cc(-c3ccc(Cl)cc3Cl)ccc2C(=O)O)c(C(=O)O)cc1F] | 2-({4-carboxy-2′,4′-dichloro-[1,1′- biphenyl]-3-yl}carbamoyl)-5- fluorobenzene-1,4-dicarboxylic acid |
| Example 80 | 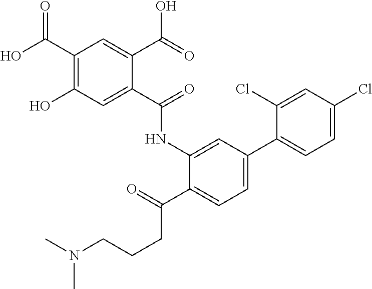 [SMILES: CN(C)CCCC(=O)c1ccc(-c2ccc(Cl)cc2Cl)cc1NC(=O)c1cc(O)c(C(=O)O)cc1C(=O)O] | 4-({2′,4′-dichloro-4-[4- (dimethylamino)butanoyl]-[1,1′-biphenyl]- 3-yl}carbamoyl)-6-hydroxybenzene-1,3- dicarboxylic acid |
| Example 79 | 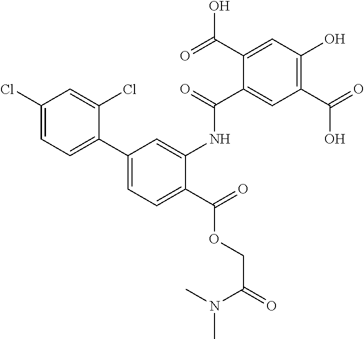 [SMILES: CN(C)C(=O)COC(=O)c1ccc(-c2ccc(Cl)cc2Cl)cc1NC(=O)c1cc(C(=O)O)c(O)cc1C(=O)O] | 2-[(2′,4′-dichloro-4- {[(dimethylcarbamoyl)methoxy]carbonyl}- [1,1′-biphenyl]-3-yl)carbamoyl]-5- hydroxybenzene-1,4-dicarboxylic acid |
| Example 78 | 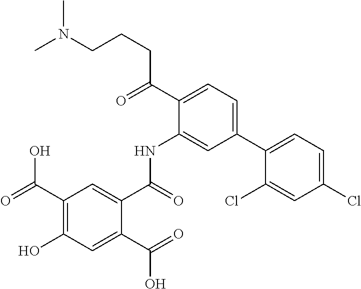 [SMILES: CN(C)CCCC(=O)c1ccc(-c2ccc(Cl)cc2Cl)cc1NC(=O)c1cc(C(=O)O)c(O)cc1C(=O)O] | 2-({2′,4′-dichloro-4-[4- (dimethylamino)butanoyl]-[1,1′-biphenyl]- 3-yl}carbamoyl)-5-hydroxybenzene-1,4- dicarboxylic acid |
| Example 77 | 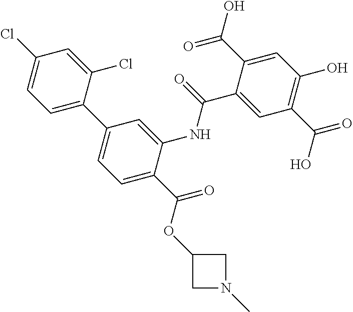 [SMILES: CN1CC(OC(=O)c2ccc(-c3ccc(Cl)cc3Cl)cc2NC(=O)c2cc(C(=O)O)c(O)cc2C(=O)O)C1] | 2-[(2′,4′-dichloro-4-{[(1-methylazetidin-3- yl)oxy]carbonyl}-[1,1′-biphenyl]-3- yl)carbamoyl]-5-hydroxybenzene-1,4- dicarboxylic acid |
| Example 76 | 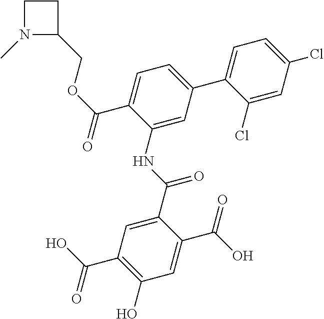 [SMILES: CN1CCC1COC(=O)c1ccc(-c2ccc(Cl)cc2Cl)cc1NC(=O)c1cc(C(=O)O)c(O)cc1C(=O)O] | 2-[(2′,4′-dichloro-4-{[(1-methylazetidin-2- yl)methoxy]carbonyl}-[1,1′-biphenyl]-3- yl)carbamoyl]-5-hydroxybenzene-1,4- dicarboxylic acid |
| Example 75 | 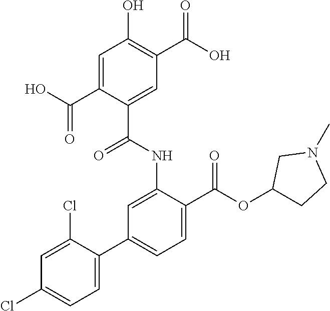 [SMILES: CN1CCC(OC(=O)c2ccc(-c3ccc(Cl)cc3Cl)cc2NC(=O)c2cc(C(=O)O)c(O)cc2C(=O)O)C1] | 2-[(2′,4′-dichloro-4-{[(1-methylpyrrolidin- 3-yl)oxy]carbonyl}-[1,1′-biphenyl]-3- yl)carbamoyl]-5-hydroxybenzene-1,4- dicarboxylic acid |
| Example 74 | 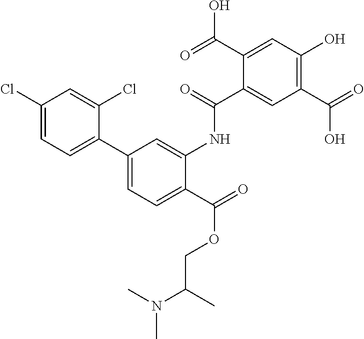 [SMILES: CC(COC(=O)c1ccc(-c2ccc(Cl)cc2Cl)cc1NC(=O)c1cc(C(=O)O)c(O)cc1C(=O)O)N(C)C] | 2-[(2′,4′-dichloro-4-{[2- (dimethylamino)propoxy]carbonyl}-[1,1′- biphenyl]-3-yl)carbmaoyl]-5- hydroxybenzene-1,4-dicarboxylic acid |
| Example 73 | 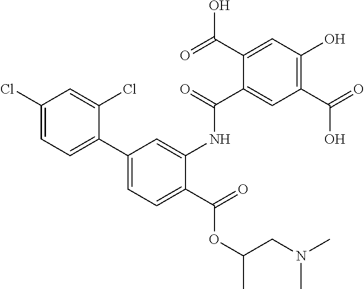 [SMILES: CC(CN(C)C)OC(=O)c1ccc(-c2ccc(Cl)cc2Cl)cc1NC(=O)c1cc(C(=O)O)c(O)cc1C(=O)O] | 2-{[2′,4′-dichloro-4-({[1- (dimethylamino)propan-2- yl]oxy}carbonyl)-[1,1′-biphenyl]-3- yl]carbamoyl}-5-hydroxybenzene-1,4- dicarboxylic acid |
| Example 72 | 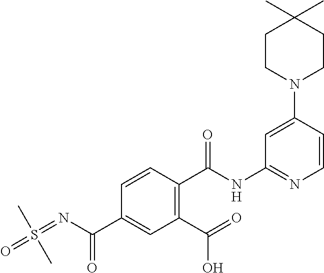 [SMILES: CC1(C)CCN(c2ccnc(NC(=O)c3ccc(C(=O)N=S(C)(C)=O)cc3C(=O)O)c2)CC1] | 5-{[dimethyl(oxo)-λ⁶- sulfanylidene]carbamoyl}-2-{[4-(4,4- dimethylpiperidin-1-yl)pyridin-2- yl]carbamoyl}benzoic acid |
| Example 71 | 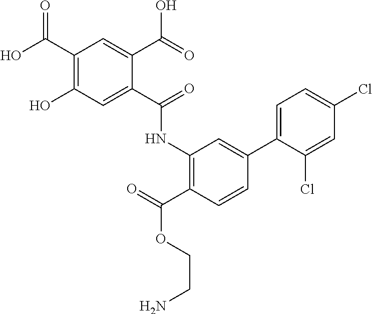 [SMILES: NCCOC(=O)c1ccc(-c2ccc(Cl)cc2Cl)cc1NC(=O)c1cc(O)c(C(=O)O)cc1C(=O)O] | 4-({4-[(2-aminoethoxy)carbonyl]-2′,4′- dichloro-[1,1′-biphenyl]-3-yl}carbamoyl)- 6-hydroxybenzene-1,3-dicarboxylic acid |
| Example 70 | 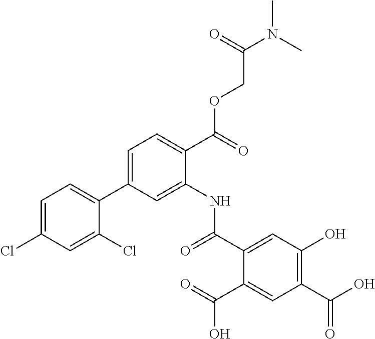 [SMILES: CN(C)C(=O)COC(=O)c1ccc(-c2ccc(Cl)cc2Cl)cc1NC(=O)c1cc(O)c(C(=O)O)cc1C(=O)O] | 4-[(2′,4′-dichloro-4- {[(dimethylcarbamoyl)methoxy]carbonyl}- [1,1′-biphenyl]-3-yl)carbamoyl]-6- hydroxybenzene-1,3-dicarboxylic acid |
| Example 69 | 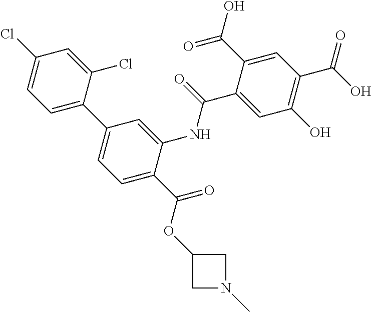 [SMILES: CN1CC(OC(=O)c2ccc(-c3ccc(Cl)cc3Cl)cc2NC(=O)c2cc(O)c(C(=O)O)cc2C(=O)O)C1] | 4-[(2′,4′-dichloro-4-{[(1-methylazetidin-3- yl)oxy]carbonyl}-[1,1′-biphenyl]-3- yl)carbamoyl]-6-hydroxybenzene-1,3- dicarboxylic acid |
| Example 68 | 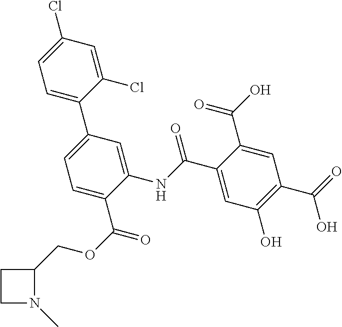 [SMILES: CN1CCC1COC(=O)c1ccc(-c2ccc(Cl)cc2Cl)cc1NC(=O)c1cc(O)c(C(=O)O)cc1C(=O)O] | 4-[(2′,4′-dichloro-4-{[(1-methylazetidin-2- yl)methoxy]carbonyl}-[1,1′-biphenyl]-3- yl)carbamoyl]-6-hydroxybenzene-1,3- dicarboxylic acid |
| Example 67 | 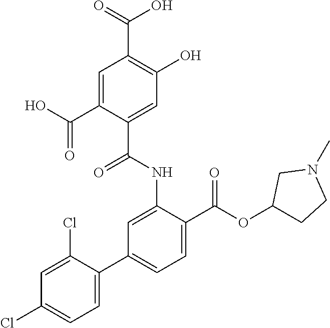 [SMILES: CN1CCC(OC(=O)c2ccc(-c3ccc(Cl)cc3Cl)cc2NC(=O)c2cc(O)c(C(=O)O)cc2C(=O)O)C1] | 4-[(2′,4′-dichloro-4-{[(1-methylpyrrolidin- 3-yl)oxy]carbonyl}-[1,1′-biphenyl]-3- yl)carbamoyl]-6-hydroxybenzene-1,3- dicarboxylic acid |
| Example 66 | 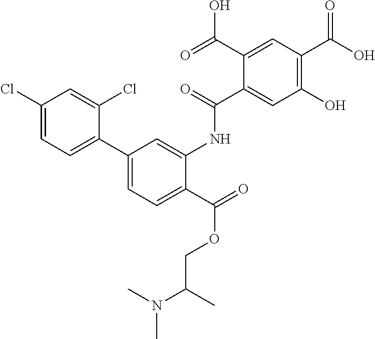 [SMILES: CC(COC(=O)c1ccc(-c2ccc(Cl)cc2Cl)cc1NC(=O)c1cc(O)c(C(=O)O)cc1C(=O)O)N(C)C] | 4-[(2′,4′-dichloro-4-{[2- (dimethylamino)propoxy]carbonyl}-[1,1′- biphenyl]-3-yl)carbamoyl]-6- hydroxybenzene-1,3-dicarboxylic acid |
| Example 65 | 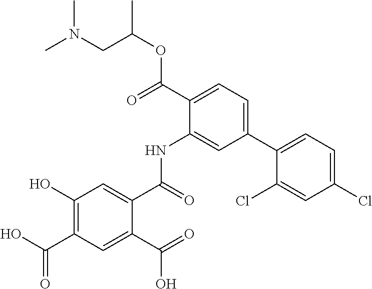 [SMILES: CC(CN(C)C)OC(=O)c1ccc(-c2ccc(Cl)cc2Cl)cc1NC(=O)c1cc(O)c(C(=O)O)cc1C(=O)O] | 4-{[2′,4′-dichloro-4-({[1- (dimethylamino)propan-2- yl]oxy}carbonyl)-[1,1′-biphenyl]-3- yl]carbamoyl}-6-hydroxybenzene-1,3- dicarboxylic acid |
| Example 64 | 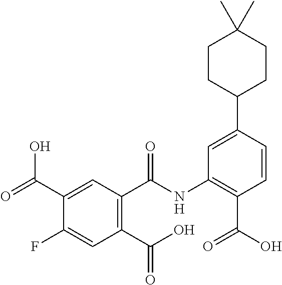 [SMILES: CC1(C)CCC(c2ccc(C(=O)O)c(NC(=O)c3cc(C(=O)O)c(F)cc3C(=O)O)c2)CC1] | 2-{[2-carboxy-5-(4,4- dimethylcyclohexyl)phenyl]carbamoyl}-5- fluorobenzene-1,4-dicarboxylic acid |
| Example 63 | 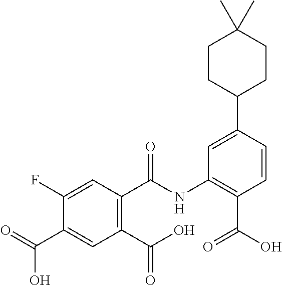 [SMILES: CC1(C)CCC(c2ccc(C(=O)O)c(NC(=O)c3cc(F)c(C(=O)O)cc3C(=O)O)c2)CC1] | 4-{[2-carboxy-5-(4,4- dimethylcyclohexyl)phenyl]carbamoyl}-6- fluorobenzene-1,3-dicarboxylic acid |
| Example 62 | 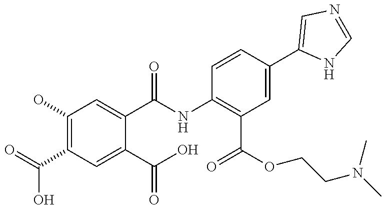 | 4-[(5-{2-azabicyclo[2.2.1]heptan-2-yl}-2- carboxyphenyl)carbamoyl]-6- hydroxybenzene-1,3-dicarboxylic acid |
| Example 61 | 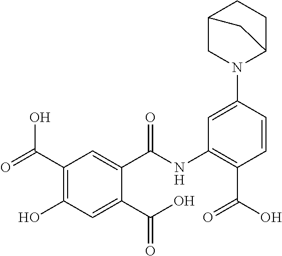 [SMILES: O=C(O)c1cc(C(=O)Nc2cc(N3CC4CCC3C4)ccc2C(=O)O)c(C(=O)O)cc1O] | 2-[(5-{2-azabicyclo[2.2.1]heptan-2-yl}-2- carboxyphenyl)carbamoyl]-5- hydroxybenzene-1,4-dicarboxylic acid |
| Example 60 | 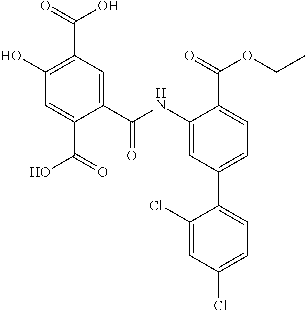 [SMILES: CCOC(=O)c1ccc(-c2ccc(Cl)cc2Cl)cc1NC(=O)c1cc(C(=O)O)c(O)cc1C(=O)O] | 2-{[2′,4′-dichloro-4-(ethoxycarbonyl)- [1,1′-biphenyl]-3-yl]carbamoyl}-5- hydroxybenzene-1,4-dicarboxylic acid |
| Example 59 | 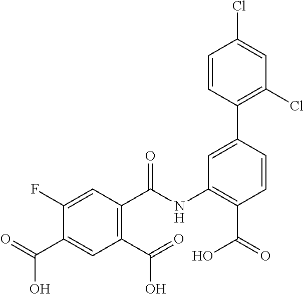 [SMILES: O=C(O)c1cc(C(=O)O)c(C(=O)Nc2cc(-c3ccc(Cl)cc3Cl)ccc2C(=O)O)cc1F] | 4-({4-carboxy-2′,4′-dichloro-[1,1′- biphenyl]-3-yl}carbamoyl)-6- fluorobenzene-1,3-dicarboxylic acid |
| Example 58 | 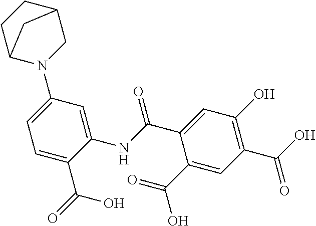 [SMILES: O=C(O)c1cc(C(=O)O)c(C(=O)Nc2cc(N3CC4CCC3C4)ccc2C(=O)O)cc1O] | 4-[(5-{2-azabicyclo[2.2.1]heptan-2-yl}-2- carboxyphenyl)carbamoyl]-6- hydroxybenzene-1,3-dicarboxylic acid |
| Example 57 | 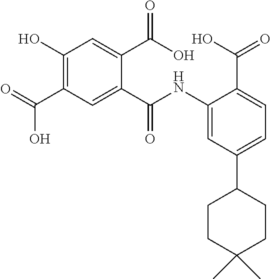 [SMILES: CC1(C)CCC(c2ccc(C(=O)O)c(NC(=O)c3cc(C(=O)O)c(O)cc3C(=O)O)c2)CC1] | 2-{[2-carboxy-5-(4,4- dimethylcyclohexyl)phenyl]carbamoyl}-5- hydroxybenzene-1,4-dicarboxylic acid |
| Example 56 | 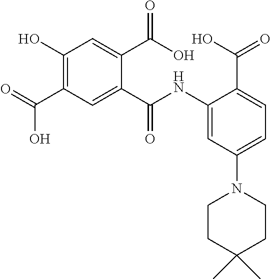 [SMILES: CC1(C)CCN(c2ccc(C(=O)O)c(NC(=O)c3cc(C(=O)O)c(O)cc3C(=O)O)c2)CC1] | 2-{[2-carboxy-5-(4,4-dimethylpiperidin-1- yl)phenyl]carbamoyl}-5-hydroxybenzene- 1,4-dicarboxylic acid |
| Example 55 | 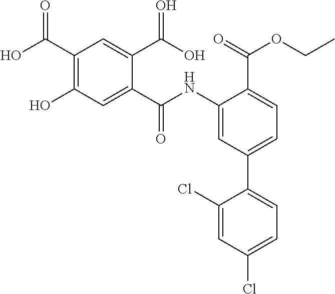 [SMILES: CCOC(=O)c1ccc(-c2ccc(Cl)cc2Cl)cc1NC(=O)c1cc(O)c(C(=O)O)cc1C(=O)O] | 4-{[2′,4′-dichloro-4-(ethoxycarbonyl)- [1,1′-biphenyl]-3-yl]carbamoyl}-6- hydroxybenzene-1,3-dicarboxylic acid |
| Example 54 | 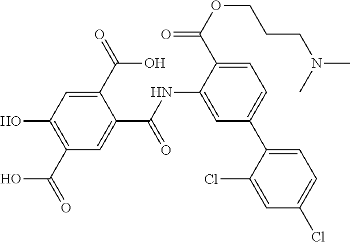 [SMILES: CN(C)CCCOC(=O)c1ccc(-c2ccc(Cl)cc2Cl)cc1NC(=O)c1cc(C(=O)O)c(O)cc1C(=O)O] | [(2′,4′-dichloro-4-{[3- (dimethylamino)propoxy]carbonyl}-[1,1′- biphenyl]-3-yl)carbamoyl]-5- hydroxybenzene-1,4-dicarboxylic acid |
| Example 53 | 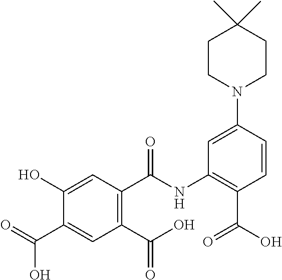 [SMILES: CC1(C)CCN(c2ccc(C(=O)O)c(NC(=O)c3cc(O)c(C(=O)O)cc3C(=O)O)c2)CC1] | 4-{[2-carboxy-5-(4,4-dimethylpiperidin-1- yl)phenyl]carbamoyl}-6-hydroxybenzene- 1,3-dicarboxylic acid |
| Example 52 | 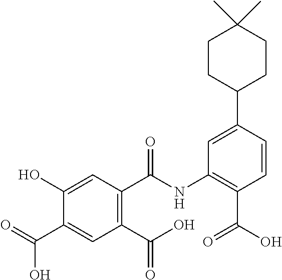 [SMILES: CC1(C)CCC(c2ccc(C(=O)O)c(NC(=O)c3cc(O)c(C(=O)O)cc3C(=O)O)c2)CC1] | 4-{[2-carboxy-5-(4,4- dimethylcyclohexyl)phenyl]carbamoyl}-6- hydroxybenzene-1,3-dicarboxylic acid |
| Example 51 | 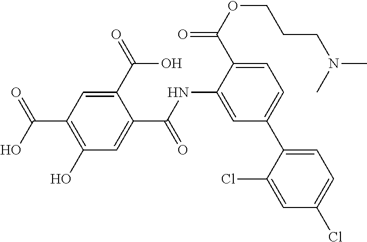 [SMILES: CN(C)CCCOC(=O)c1ccc(-c2ccc(Cl)cc2Cl)cc1NC(=O)c1cc(O)c(C(=O)O)cc1C(=O)O] | 4-[(2′,4′-dichloro-4-{[3- (dimethylamino)propoxy]carbonyl}-[1,1′- biphenyl]-3-yl)carbamoyl]-6- hydroxybenzene-1,3-dicarboxylic acid |
| Example 50 | 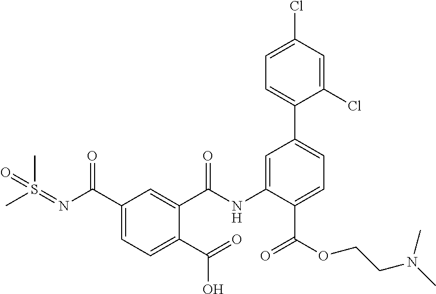 [SMILES: CN(C)CCOC(=O)c1ccc(-c2ccc(Cl)cc2Cl)cc1NC(=O)c1cc(C(=O)N=S(C)(C)=O)ccc1C(=O)O] | 2-[(2′,4′-dichloro-4-{[2- (dimethylamino)ethoxy]carbonyl}-[1,1′- biphenyl]-3-yl)carbamoyl]-4- {[dimethyl(oxo)-λ⁶- sulfanylidene]carbamoyl}benzoic acid |
| Example 49 | 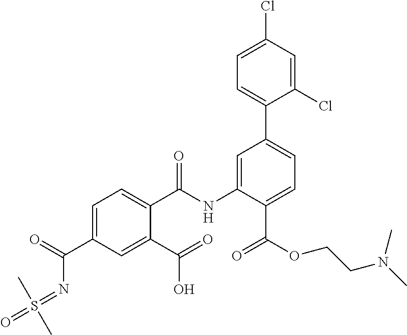 [SMILES: CN(C)CCOC(=O)c1ccc(-c2ccc(Cl)cc2Cl)cc1NC(=O)c1ccc(C(=O)N=S(C)(C)=O)cc1C(=O)O] | 2-[(2′,4′-dichloro-4-{[2- (dimethylamino)ethoxy]carbonyl}-[1,1′- biphenyl]-3-yl)carbamoyl]-5- {[dimethyl(oxo)-λ⁶- sulfanylidene]carbamoyl}benzoic acid |
| Example 46 | 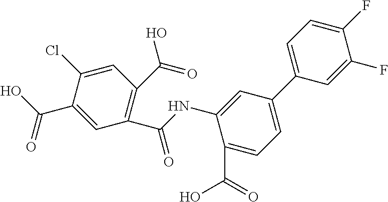 [SMILES: O=C(O)c1cc(C(=O)Nc2cc(-c3ccc(F)c(F)c3)ccc2C(=O)O)c(C(=O)O)cc1Cl] | 2-({4-carboxy-3′,4′-difluoro-[1,1′- biphenyl]-3-yl}carbamoyl)-5- chlorobenzene-1,4-dicarboxylic acid |
| Example 45 | 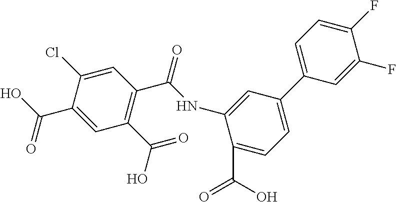 [SMILES: O=C(O)c1cc(C(=O)O)c(C(=O)Nc2cc(-c3ccc(F)c(F)c3)ccc2C(=O)O)cc1Cl] | 4-({4-carboxy-3′,4′-difluoro-[1,1′- biphenyl]-3-yl}carbamoyl)-6- chlorobenzene-1,3-dicarboxylic acid |
| Example 44 | 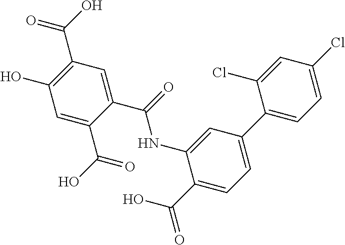 [SMILES: O=C(O)c1cc(C(=O)Nc2cc(-c3ccc(Cl)cc3Cl)ccc2C(=O)O)c(C(=O)O)cc1O] | 2-({4-carboxy-2′,4′-dichloro-[1,1′- biphenyl]-3-yl}carbamoyl)-5- hydroxybenzene-1,4-dicarboxylic acid |
| Example 43 | 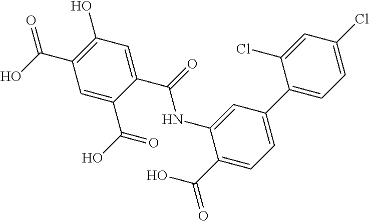 [SMILES: O=C(O)c1cc(C(=O)O)c(C(=O)Nc2cc(-c3ccc(Cl)cc3Cl)ccc2C(=O)O)cc1O] | 4-({4-carboxy-2′,4′-dichloro-[1,1′- biphenyl]-3-yl}carbamoyl)-6- hydroxybenzene-1,3-dicarboxylic acid |
| Example 42 | 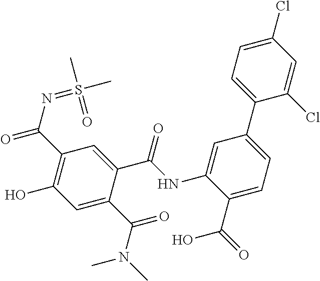 [SMILES: CN(C)C(=O)c1cc(O)c(C(=O)N=S(C)(C)=O)cc1C(=O)Nc1cc(-c2ccc(Cl)cc2Cl)ccc1C(=O)O] | 2′,4′-dichloro-3-(5-{[dimethyl(oxo)-λ⁶- sulfanylidene]carbamoyl}-2- (dimethylcarbamoyl)-4- hydroxybenzamido)-[1,1′-biphenyl]-4- carboxylic acid |
| Example 41 | 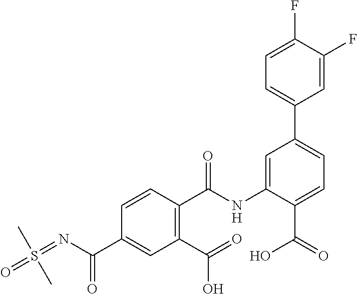 [SMILES: CS(C)(=O)=NC(=O)c1ccc(C(=O)Nc2cc(-c3ccc(F)c(F)c3)ccc2C(=O)O)c(C(=O)O)c1] | 3-(2-carboxy-4-{[dimethyl(oxo)-λ⁶- sulfanylidene]carbamoyl}benzamido)- 3′,4′-difluoro-[1,1′-biphenyl]-4-carboxylic acid |
| Example 40 | 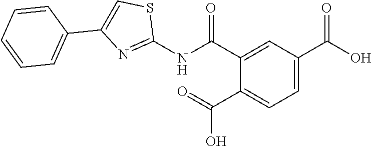 [SMILES: O=C(O)c1ccc(C(=O)O)c(C(=O)Nc2nc(-c3ccccc3)cs2)c1] | 2-[(4-phenyl-1,3-thiazol-2- yl)carbamoyl]benzene-1,4-dicarboxylic acid |
| Example 39 | 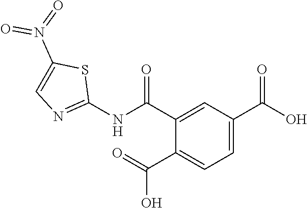 [SMILES: O=C(O)c1ccc(C(=O)O)c(C(=O)Nc2ncc([N+](=O)[O-])s2)c1] | 2-[(5-nitro-1,3-thiazol-2- yl)carbamoyl]benzene-1,4-dicarboxylic acid |
| Example 38 | 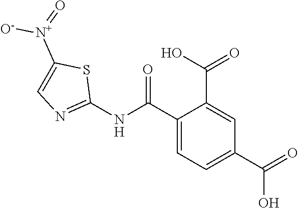 [SMILES: O=C(O)c1ccc(C(=O)Nc2ncc([N+](=O)[O-])s2)c(C(=O)O)c1] | 4-[(5-nitro-1,3-thiazol-2- yl)carbamoyl]benzene-1,3-dicarboxylic acid |
| Example 37 | 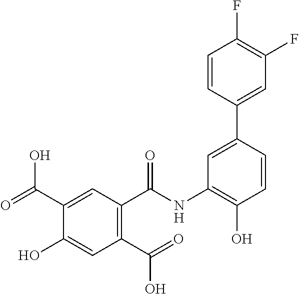 [SMILES: O=C(O)c1cc(C(=O)Nc2cc(-c3ccc(F)c(F)c3)ccc2O)c(C(=O)O)cc1O] | 2-({3′,4′-difluoro-4-hydroxy-[1,1′- biphenyl]-3-yl}carbamoyl)-5- hydroxybenzene-1,4-dicarboxylic acid |
| Example 36 | 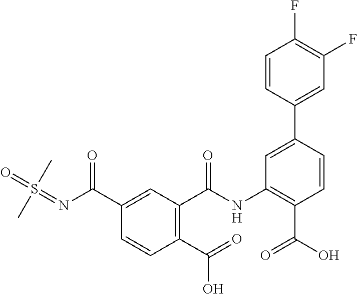 [SMILES: CS(C)(=O)=NC(=O)c1ccc(C(=O)O)c(C(=O)Nc2cc(-c3ccc(F)c(F)c3)ccc2C(=O)O)c1] | 3-(2-carboxy-5-{[dimethyl(oxo)-λ⁶- sulfanylidene]carbamoyl}benzamido)- 3′,4′-difluoro-[1,1′-biphenyl]-4-carboxylic acid |
| Example 35 | 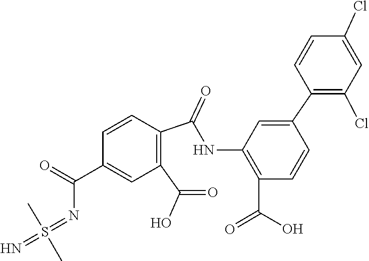 [SMILES: CS(C)(=N)=NC(=O)c1ccc(C(=O)Nc2cc(-c3ccc(Cl)cc3Cl)ccc2C(=O)O)c(C(=O)O)c1] | 3-(2-carboxy-4- {[imino(methyl)methylidene-λ⁶- sulfanyl]carbamoyl}benzamido)-2′,4′- dichloro-[1,1′-biphenyl]-4-carboxylic acid |
| Example 34 | 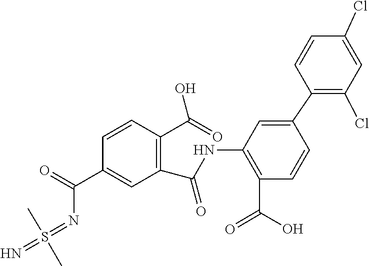 [SMILES: CS(C)(=N)=NC(=O)c1ccc(C(=O)O)c(C(=O)Nc2cc(-c3ccc(Cl)cc3Cl)ccc2C(=O)O)c1] | 3-(2-carboxy-5- {[imino(methyl)methylidene-λ⁶- sulfanyl]carbamoyl}benzamido)-2′,4′- dichloro-[1,1′-biphenyl]-4-carboxylic acid |
| Example 33 | 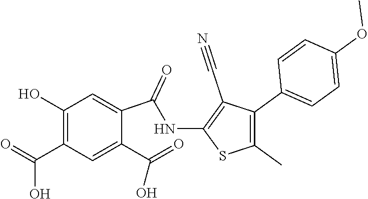 [SMILES: COc1ccc(-c2c(C)sc(NC(=O)c3cc(O)c(C(=O)O)cc3C(=O)O)c2C#N)cc1] | 4-((3-cyano-4-(4-methoxyphenyl)-5- methylthiophen-2-yl)carbamoyl)-6- hydroxyisophthalic acid |
| Example 32 | 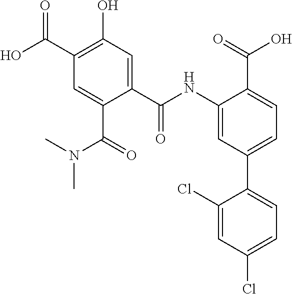 [SMILES: CN(C)C(=O)c1cc(C(=O)O)c(O)cc1C(=O)Nc1cc(-c2ccc(Cl)cc2Cl)ccc1C(=O)O] | 3-[4-carboxy-2-(dimethylcarbamoyl)-5- hydroxybenzamido]-2′,4′-dichloro-[1,1′- biphenyl]-4-carboxylic acid |
| Example 30 | 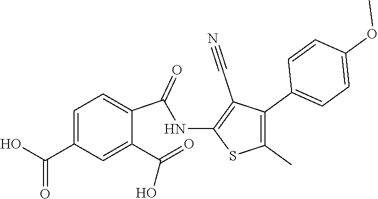 [SMILES: COc1ccc(-c2c(C)sc(NC(=O)c3ccc(C(=O)O)cc3C(=O)O)c2C#N)cc1] | 4-{[3-cyano-4-(4-methoxyphenyl)-5- methylthiophen-2-yl]carbamoyl}benzene- 1,3-dicarboxylic acid |
| Example 29 | 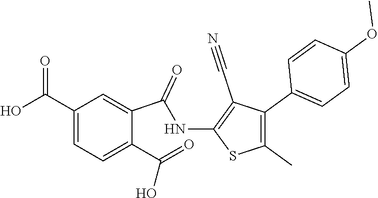 [SMILES: COc1ccc(-c2c(C)sc(NC(=O)c3cc(C(=O)O)ccc3C(=O)O)c2C#N)cc1] | 2-{[3-cyano-4-(4-methoxyphenyl)-5- methylthiophen-2-yl]carbamoyl}benzene- 1,4-dicarboxylic acid |
| Example 28 | 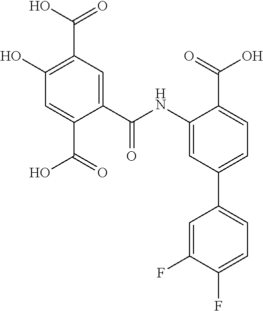 [SMILES: O=C(O)c1cc(C(=O)Nc2cc(-c3ccc(F)c(F)c3)ccc2C(=O)O)c(C(=O)O)cc1O] | 2-({4-carboxy-3′,4′-difluoro-[1,1′- biphenyl]-3-yl}carbamoyl)-5- hydroxybenzene-1,4-dicarboxylic acid |
| Example 27 | 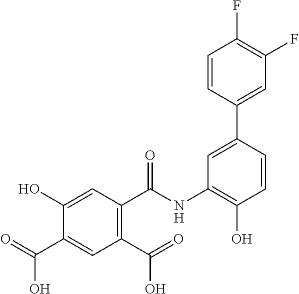 [SMILES: O=C(O)c1cc(C(=O)O)c(C(=O)Nc2cc(-c3ccc(F)c(F)c3)ccc2O)cc1O] | 4-({3′,4′-difluoro-4-hydroxy-[1,1′- biphenyl]-3-yl}carbamoyl)-6- hydroxybenzene-1,3-dicarboxylic acid |
| Example 26 | 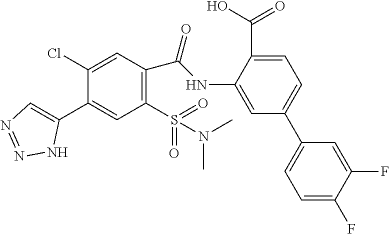 [SMILES: CN(C)S(=O)(=O)c1cc(-c2cnn[nH]2)c(Cl)cc1C(=O)Nc1cc(-c2ccc(F)c(F)c2)ccc1C(=O)O] | 3-[5-chloro-2-(dimethylsulfamoyl)-4-(2H- 1,2,3-triazol-4-yl)benzamido]-3′,4′- difluoro-[1,1′-biphenyl]-4-carboxylic acid |
| Example 23 | 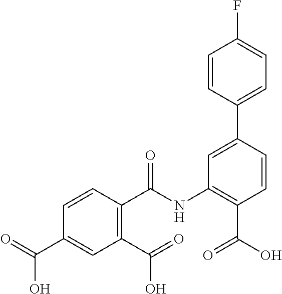 [SMILES: O=C(O)c1ccc(C(=O)Nc2cc(-c3ccc(F)cc3)ccc2C(=O)O)c(C(=O)O)c1] | 4-({4-carboxy-4′-fluoro-[1,1′-biphenyl]-3- yl}carbamoyl)benzene-1,3-dicarboxylic acid |
| Example 22 | 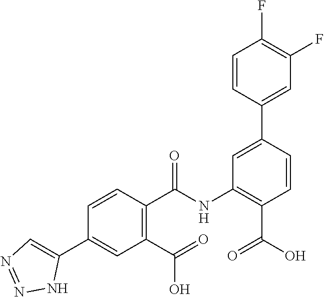 [SMILES: O=C(O)c1ccc(-c2ccc(F)c(F)c2)cc1NC(=O)c1ccc(-c2cnn[nH]2)cc1C(=O)O] | 3-[2-carboxy-4-(2H-1,2,3-triazol-4- yl)benzamido]-3′,4′-difluoro-[1,1′- biphenyl]-4-carboxylic acid |
| Example 21 | 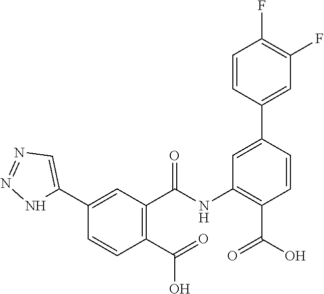 [SMILES: O=C(O)c1ccc(-c2ccc(F)c(F)c2)cc1NC(=O)c1cc(-c2cnn[nH]2)ccc1C(=O)O] | 3-[2-carboxy-5-(2H-1,2,3-triazol-4- yl)benzamido]-3′,4′-difluoro-[1,1′- biphenyl]-4-carboxylic acid |
| Example 20 | 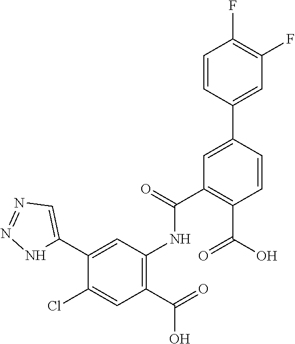 [SMILES: O=C(O)c1cc(Cl)c(-c2cnn[nH]2)cc1NC(=O)c1cc(-c2ccc(F)c(F)c2)ccc1C(=O)O] | 3-{[2-carboxy-4-chloro-5-(2H-1,2,3- triazol-4-yl)phenyl]carbamoyl}-3′,4′- difluoro-[1,1′-biphenyl]-4-carboxylic acid |
| Example 19 | 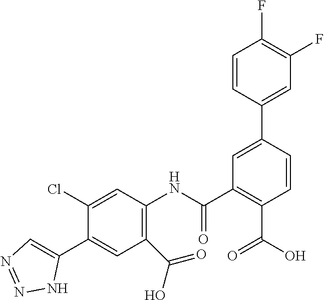 [SMILES: O=C(O)c1cc(-c2cnn[nH]2)c(Cl)cc1NC(=O)c1cc(-c2ccc(F)c(F)c2)ccc1C(=O)O] | 3-{[2-carboxy-5-chloro-4-(2H-1,2,3- triazol-4-yl)phenyl]carbamoyl}-3′,4′- difluoro-[1,1′-biphenyl]-4-carboxylic acid |
| Example 18 | 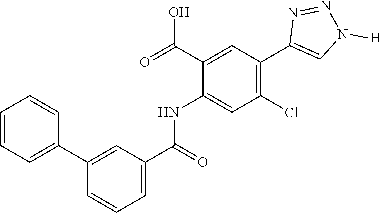 [SMILES: O=C(Nc1cc(Cl)c(-c2c[nH]nn2)cc1C(=O)O)c1cccc(-c2ccccc2)c1] | 2-{[1,1′-biphenyl]-3-amido}-4-chloro-5- (2H-1,2,3-triazol-4-yl)benzoic acid |
| Example 17 | 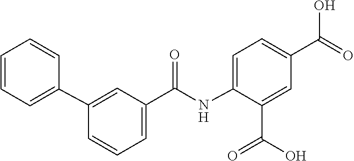 [SMILES: O=C(O)c1ccc(NC(=O)c2cccc(-c3ccccc3)c2)c(C(=O)O)c1] | 4-{[1,1′-biphenyl]-3-amido}benzene-1,3- dicarboxylic acid |
| Example 16 | 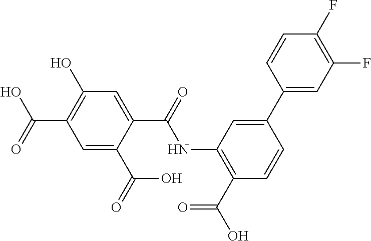 [SMILES: O=C(O)c1cc(C(=O)O)c(C(=O)Nc2cc(-c3ccc(F)c(F)c3)ccc2C(=O)O)cc1O] | 4-({4-carboxy-3′,4′-difluoro-[1,1′- biphenyl]-3-yl}carbamoyl)-6- hydroxybenzene-1,3-dicarboxylic acid |
| Example 15 | 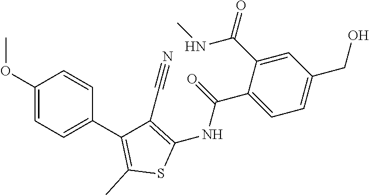 [SMILES: CNC(=O)c1cc(CO)ccc1C(=O)Nc1sc(C)c(-c2ccc(OC)cc2)c1C#N] | N1-[3-cyano-4-(4-methoxyphenyl)-5- methylthiophen-2-yl]-4-(hydroxymethyl)- N2-methylbenzene-1,2-dicarboxamide |
| Example 14 | 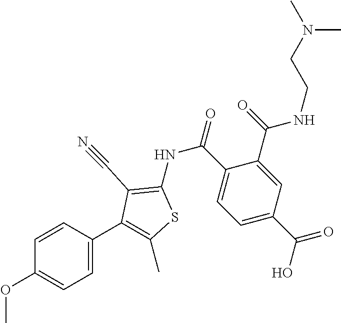 [SMILES: COc1ccc(-c2c(C)sc(NC(=O)c3ccc(C(=O)O)cc3C(=O)NCCN(C)C)c2C#N)cc1] | 4-{[3-cyano-4-(4-methoxyphenyl)-5- methylthiophen-2-yl]carbamoyl}-3-{[2- (dimethylamino)ethyl]carbamoyl}benzoic acid |
| Example 12 | 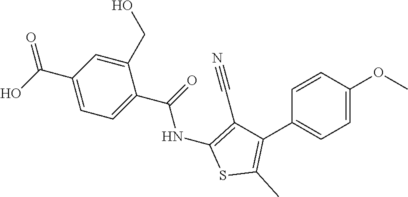 [SMILES: COc1ccc(-c2c(C)sc(NC(=O)c3ccc(C(=O)O)cc3CO)c2C#N)cc1] | 4-{[3-cyano-4-(4-methoxyphenyl)-5- methylthiophen-2-yl]carbamoyl}-3- (hydroxymethyl)benzoic acid |
| Example 11 | 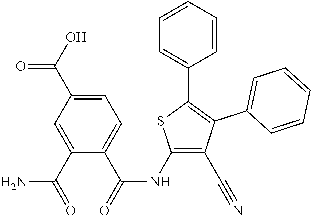 [SMILES: N#Cc1c(NC(=O)c2ccc(C(=O)O)cc2C(N)=O)sc(-c2ccccc2)c1-c1ccccc1] | 3-carbamoyl-4-[(3-cyano-4,5- diphenylthiophen-2- yl)carbamoyl]benzoic acid |
| Example 10 | 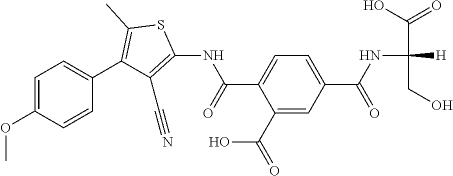 [SMILES: COc1ccc(-c2c(C)sc(NC(=O)c3ccc(C(=O)N[C@@H](CO)C(=O)O)cc3C(=O)O)c2C#N)cc1] | 5-[(1-carboxy-2- hydroxyethyl)carbamoyl]-2-{[3-cyano-4- (4-methoxyphenyl)-5-methylthiophen-2- yl]carbamoyl}benzoic acid |
| Example 9 | 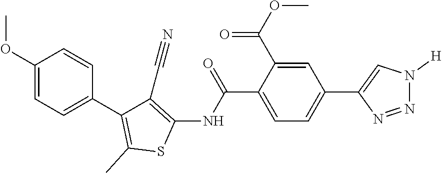 [SMILES: COC(=O)c1cc(-c2c[nH]nn2)ccc1C(=O)Nc1sc(C)c(-c2ccc(OC)cc2)c1C#N] | methyl 2-{[3-cyano-4-(4-methoxyphenyl)- 5-methylthiophen-2-yl]carbamoyl}-5-(2H- 1,2,3-triazol-4-yl)benzoate |
| Example 8 | 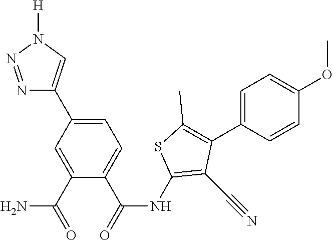 [SMILES: COc1ccc(-c2c(C)sc(NC(=O)c3ccc(-c4c[nH]nn4)cc3C(N)=O)c2C#N)cc1] | N1-[3-cyano-4-(4-methoxyphenyl)-5- methylthiophen-2-yl]-4-(2H-1,2,3-triazol- 4-yl)benzene-1,2-dicarboxamide |
| Example 7 | 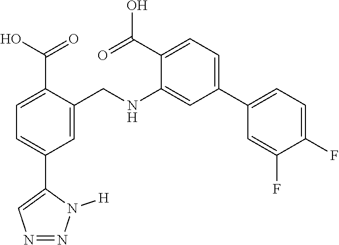 [SMILES: O=C(O)c1ccc(-c2cnn[nH]2)cc1CNc1cc(-c2ccc(F)c(F)c2)ccc1C(=O)F] | 3-({[2-carboxy-5-(2H-1,2,3-triazol-4- yl)phenyl]methyl}amino)-3′,4′-difluoro- [1,1′-biphenyl]-4-carboxylic acid |
| Example 6 | 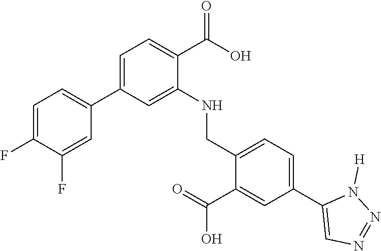 [SMILES: O=C(O)c1cc(-c2cnn[nH]2)ccc1CNc1cc(-c2ccc(F)c(F)c2)ccc1C(=O)O] | 3-({[2-carboxy-4-(2H-1,2,3-triazol-4- yl)phenyl]methyl}amino)-3′,4′-difluoro- [1,1′-biphenyl]-4-carboxylic acid |
| Example 5 | 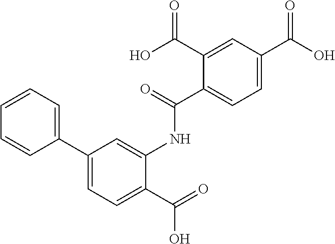 [SMILES: O=C(O)c1ccc(C(=O)Nc2cc(-c3ccccc3)ccc2C(=O)O)c(C(=O)O)c1] | 4-({4-carboxy-[1,1′-biphenyl]-3- yl}carbamoyl)benzene-1,3-dicarboxylic acid |
| Example 4 | 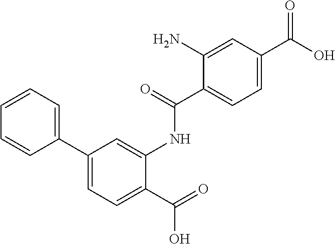 [SMILES: Nc1cc(C(=O)O)ccc1C(=O)Nc1cc(-c2ccccc2)ccc1C(=O)O] | 3-(2-amino-4-carboxybenzamido)-[1,1′- biphenyl]-4-carboxylic acid |
| Example 1 | 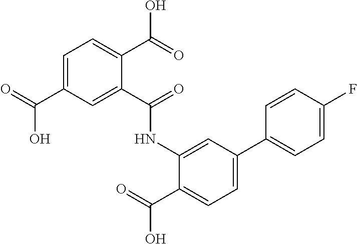 [SMILES: O=C(O)c1ccc(C(=O)O)c(C(=O)Nc2cc(-c3ccc(F)cc3)ccc2C(=O)O)c1] | 2-({4-carboxy-4′-fluoro-[1,1′-biphenyl]-3- yl}carbamoyl)benzene-1,4-dicarboxylic acid |

Substantial share of compounds, described by items 162-194 below, are derivatives of 2-carbamoylbenzoic acid and can be obtained by reaction of ring opening from correspondent phthalimide structures, described by items 1-161. Moreover, some of these phthalimides could be used as prodrugs for correspondent 2-carbamoylbenzoic acid-derivatives.

In some embodiments, this invention relates to kits, methods, compositions, pharmaceutical compositions, uses, PFKFB3 inhibitor for use, medium etc., comprising any one of PFKFB3 inhibitors, selected from new inhibitors disclosed herein and those which are known in the art, their structural analog, functional analog, derivative, N-oxide, prodrug, solvate, tautomer, stereoisomer, racemate, physiologically acceptable salt, including mixtures thereof in all ratios of PFKFB3 inhibitor, including but not limited to those described in items below.

Preparation of the Compounds

The compounds used in the reactions described herein are made according to known organic synthesis techniques, starting from commercially available chemicals and/or from compounds described in the chemical literature. “Commercially available chemicals” are obtained from standard commercial sources. The following non-exhaustive and non-exclusive list of commercial of the commercial providers is given for example and reference only, the compounds used for this invention could have been obtained from other providers: Acros Organics (Geel, Belgium), Aldrich Chemical (Milwaukee, Wis., including Sigma Chemical and Fluka), Alfa Aesar (Heysham, UK), Alfa Chemistry (Holtsville, N.Y.), Angene International Limited (London, UK), Apin Chemicals Ltd. (Milton Park, UK), Apollo Scientific Ltd (Stockport, UK), Ark Pharm, Inc. (Libertyville, Ill.), Aurora Fine Chemicals LLC (San Diego, Calif.), AURUM Pharmatech LLC (Franklin Park, N.J.), Avocado Research (Lancashire, U.K.), BDH Inc. (Toronto, Canada), Bionet (Cornwall, U.K.), Chem-Impex International (Wood Dale, Ill.), Chemservice Inc. (West Chester, Pa.), Combi-blocks, Inc (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Fluorochem Ltd (Hadfield, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Ryan Scientific, Inc. (Mount Pleasant, S.C.), Santa Cruz Biotechnology (Dallas, Tex.), Spectrum Chemicals (Gardena, Calif.), Sundia Meditech, (Shanghai, China), Suzhou Devi Pharma Technology Co. Ltd. (Suzhou, China),TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

Further Forms of Compounds Disclosed Herein

Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein.

In some situations, the compounds described herein possess one or more chiral centers and each center exists in the R configuration, or S configuration. In some embodiments, the compounds described herein possess three chiral centers and each center exists in the R configuration, or S configuration. In some embodiments, the compounds described herein possess four chiral centers and each center exists in the R configuration, or S configuration. In some embodiments, the compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt thereof is prepared by any suitable method.

In some embodiments, the compounds described herein are labeled by other means, including, but not limited to, the use of chromophores or fluorescent moieties, bioluminescent labels, or chemiluminescent labels.

Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

Prodrugs

In some embodiments, the compounds described herein exist as prodrugs. A prodrug refers to a compound that is converted into a parent compound in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity (an acid). A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, Methods and Principles in Medicinal Chemistry Prodrugs and Targeted DeliveryTowards Better ADME Properties, Volume 47 by Jarkko Rautio (Editor), Jarkko Rautio, Editor-Jarkko Rautio, Raimund Mannhold, Hugo Kubinyi, Gerd Folkers Hardcover, Published 2011 by Wiley-Vch ISBN-13: 978-3-527-32603-7, ISBN: 3-527-32603-0 Prodrugs: Challenges and Rewards Editors: Stella, V., Borchardt, R., Hageman, M., Oliyai, R., Maag, H., Tilley, J. (Eds.), Springer, Vol I-V, 2007, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs. In some embodiments, compounds described herein are prepared as substituted alkyl ester prodrugs.

Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, twenty-first Ed (Lippincott Williams & Wilkins 2012); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste. Typical compositions and dosage forms comprise one or more excipients. Suitable excipients can be those well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents. Pharmaceutical compositions which can be used orally include tablets, push-fit capsules made of gelatin, as well as soft, sealed capsules made of gelatin and a plasticizer, such as glycerol or sorbitol. Typical oral dosage forms provided herein are prepared by combining a compound in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically (non-systemic administration). This includes the application of a compound of the present invention externally to the epidermis or the buccal cavity and the instillation of such a compound into the ear, eye and nose, such that the compound does not significantly enter the bloodstream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In some embodiments, the disclosure contemplates administration of a pharmaceutical composition comprising a PFKFB3 modulator that binds to, inhibits, or degrades a PFKFB3. Administration in vivo includes administration to an animal model of disease, such as an animal model of neurodegeneration, or administration to a subject in need thereof. Suitable cells, tissues, or subjects include animals, such as companion animals, livestock, zoo animals, endangered species, rare animals, non-human primates, and humans. Exemplary companion animals include dogs and cats.

In some embodiments, for delivery in vitro, such as to and/or around cells or tissues in culture, compositions may be added to the culture media, such as to contact the microenvironment or contact soluble material in the culture media or to contact the cell or even to penetrate the cell. The desired site of activity influences the delivery mechanism and means for administering the compositions.

In some embodiments, for delivery in vivo, such as to cells or tissues in vivo (including to the microenvironment of cells and tissue) and/or to a subject in need thereof, numerous methods of administration are envisioned. The particular method may be selected based on the particle composition and the particular application and the patient. Various delivery systems can be used to administer PFKFB3 inhibitors of the disclosure. Any such methods may be used to administer any of the PFKFB3 inhibitors described herein. Methods of introduction can be enteral or parenteral, including but not limited to, intradermal, intramuscular, intraperitoneal, intramyocardial, intravenous, subcutaneous, pulmonary, intranasal, intraocular, epidural, and oral routes. A composition of the disclosure may be administered by any convenient route, for example, by infusion or bolus injection, by absorption through epithelial or mucocutaneous linings {e.g., oral mucosa, rectal and intestinal mucosa, etc.) and may be administered together (either concurrently or consecutively) with other biologically active PFKFB3 inhibitors. Administration can be systemic or local.

In certain embodiments, a composition is administered intravenously, such as by bolus inject or infusion. In certain embodiments, a composition is administered orally, subcutaneously, intramuscularly or intraperitoneally. In certain embodiments, it may be desirable to administer a composition of the disclosure locally to the area in need of treatment (e.g., directly to the brain). Other methods of delivery via the hepatic portal vein are also contemplated.

In certain embodiments, compositions of the disclosure are administered by intravenous infusion. In certain embodiments, the a composition is infused over a period of at least 10, at least 15, at least 20, or at least 30 minutes. In other embodiments, the PFKFB3 inhibitor is infused over a period of at least 60, 90, or 120 minutes. Regardless of the infusion period, the disclosure contemplates that, in certain embodiments, each infusion is part of an overall treatment plan where PFKFB3 inhibitor is administered according to a regular schedule (e.g., weekly, monthly, etc.) for some period of time. However, in other embodiments, a composition is delivered by bolus injection, e.g., as part of an overall treatment plan where PFKFB3 inhibitor is administered according to a regular schedule for some period of time.

For any of the foregoing, it is contemplated that compositions of the disclosure (include one PFKFB3 inhibitor or a combination of two or more such PFKFB3 inhibitors) may be administered in vitro or in vivo via any suitable route or method. Compositions may be administered as part of a therapeutic regimen where a composition is administered one time or multiple times, including according to a particular schedule. Moreover, it is contemplated that the compositions of the disclosure will be formulated as appropriate for the route of administration and particular application. The disclosure contemplates any combination of the foregoing features, as well as combinations with any of the aspects and embodiments of the disclosure described herein.

In some embodiments, the foregoing applies to any compositions (e.g., a particle or plurality of particles) of the disclosure, used alone or in combination, and used for any of the methods described herein. The disclosure specifically contemplates any combination of the features of such compositions of the disclosure, compositions, and methods with the features described for the various pharmaceutical compositions and routes of administration described in this section and below.

In some embodiments, this disclosure provides a medication or pharmaceutical composition comprising PFKFB3 inhibitor described in this disclosure or its structural or functional analog or its prodrug, solvate, tautomer or stereoisomer thereof as well as the physiologically acceptable salts of each of the foregoing, including mixtures thereof in all ratios.

In some embodiments, this disclosure provides pharmaceutical compositions comprising a PFKFB3 inhibitor; and, at least one pharmaceutically acceptable excipient, wherein the agent is described in this disclosure, including but not limited to agents described as PFKFB3 inhibitor in this application or its structural or functional or SAR analog or prodrug. In some embodiments this invention is a molecule or particle having at least 75%, 80%, 85%, 90%, 95%, 99% similarity to at least one of the molecules described in this disclosure as PFKFB3 inhibitor or with the PFKFB3 binding portion thereof, optionally for use as anti-aging treatment or neuroprotective treatment.

In some embodiment this invention is a molecule or other agent obtained by the in-vitro or in-silico or ex-vivo screening for binding or inhibition or degradation or deactivation of PFKFB3.

In some instances, the pharmaceutical composition described herein is formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. Where the pharmaceutical composition described herein is administered by infusion, it can be dispensed, for example, with an infusion bottle containing sterile pharmaceutical grade water or saline. Where the pharmaceutical composition described herein is administered by injection, an ampule of sterile water for injection or saline can be provided so that the enzyme or enzyme and antioxidant and the carrier can be mixed prior to administration. One of the many possible forms of this invention can be a Lyophilized Concentrate for Intravenous (IV) Injection.

Non-limiting examples of pharmaceutical compositions of this disclosure are shown in examples

The amount of pharmaceutical composition described herein that is effective for treating a corresponding disease or condition can be determined using standard clinical or pharmacokinetic techniques known to those with skill in the art. In addition, in vitro or in vivo assays can optionally be employed to help identify optimal dosage ranges. The precise dose to be employed can also depend on the route of administration, the disease or condition, the seriousness of the corresponding disease or condition being treated, as well as various physical factors related to the individual being treated, and can be decided according to the judgment of a health-care practitioner. For example, any agent (PFKFB3 inhibitor) or composition of this disclosure, in an amount ranging from about 0.05 μg/kg to about 100 mg/kg of a patient's body weight or 0.01 to about 1000 mg/kg of total body weight per day, or from about 0.1 to about 100 mg/kg of total body weight per day, or from about 0.5 to about 15 mg/kg of total body weight per day, or from about 1 mg/kg to about 50 mg/kg of total body weight, which may be administered in one or multiple doses per day or per week or per month or per 6 months or per year or per 3 years or per 8 years or per 12 years or once in a lifetime. Equivalent dosages can be administered over various time periods including, but not limited to, about every 2 hours, about every 4 hours, about every 8 hours, about every 12 hours, about every 24 hours, about every 36 hours, about every 48 hours, about every 72 hours, about every week, about every two weeks, about every three weeks, about every month, and about every two months or every 6 months or every year or every 3 years or every 8 years or every 12 years or once in a lifetime or by periods lifelong as decided by practitioner or patient. The number and frequency of dosages corresponding to a completed course of therapy can be determined according to the judgment of a health-care practitioner.

In some embodiments, the pharmaceutical composition and formulations described herein are administered to a subject by any suitable administration route, including but not limited to, parenteral (e.g., intravenous, subcutaneous, intramuscular), intradermal, intraperitoneal, subcutaneous, intranasal, epidural, sublingual, intravaginal, rectal, by inhalation, topical intracerebral, oral, intranasal, buccal, rectal, or transdermal administration routes. For example, in some instances, the pharmaceutical composition described herein is administered locally. This is achieved, for example, by local infusion during surgery, by injection, by means of a catheter, by means of a suppository or enema, or by means of an implant, the implant being of a porous, non-porous, or gelatinous material, including membranes, such as sialastic membranes, or fibers. In some situations, the pharmaceutical composition described herein is introduced into the central nervous system, circulatory system or gastrointestinal tract by any suitable route, including intraventricular, intrathecal injection, paraspinal injection, epidural injection, enema, and by injection adjacent to a peripheral nerve. Pulmonary administration can also be employed, e.g., by use of an inhaler or nebulizer, and formulation with an aerosolizing agent, or via perfusion in a fluorocarbon or synthetic pulmonary surfactant.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some embodiments, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some embodiments, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some embodiments, the pharmaceutical formulations further include diluents which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some embodiments, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some embodiments, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In some embodiments, the modulator is a small molecule inhibitor.

In some embodiments, the modulator affects the target protein or mimics the effect of PFKFB3 inhibition, degradation or reduction by contacting at least one effector upstream or downstream of PFKFB3.

It should be understood that in addition to the ingredients particularly mentioned above, the compounds and compositions described herein may include other agents conventional in the art having regard to the type of formulation in question, for example those suitable for oral administration may include flavoring agents.

It should be understood that in addition to the ingredients particularly mentioned above, the compounds and compositions described herein may include other agents conventional in the art having regard to the type of formulation in question, for example those suitable for oral administration may include flavoring agents.

Use for Manufacturing of Medicament and Methods of Manufacturing

Described herein are uses of compounds described herein or a pharmaceutically acceptable salt thereof or pharmaceutical composition comprising such compounds for manufacturing a medicament for the treatment or prophylaxis of a disease or condition where the modulation of PFKFB3 and/or PFKFB4 has beneficial effect, including but not limited to at least one of the diseases or conditions mentioned in this application. Described herein are also uses of compounds described herein or a pharmaceutically acceptable salt thereof or pharmaceutical composition comprising such compounds for manufacturing a medicament for the treatment of a disease or condition where the modulation of PFKFB3 and/or PFKFB4 has beneficial effect, including but not limited to at least one of the diseases or conditions mentioned in this application.

Described herein are uses of compounds described herein or a pharmaceutically acceptable salt thereof or pharmaceutical composition comprising such compounds for manufacturing a medicament for the prophylaxis of a disease or condition where the modulation of PFKFB3 and/or PFKFB4 has beneficial effect, including but not limited to at least one of the diseases or conditions mentioned in this application.

Methods of Treatment and Treatment Regimens

Described herein are also methods of inhibition of the glycolysis in a cell, comprising contacting the cell with compounds described herein or a pharmaceutically acceptable salt thereof or pharmaceutical composition comprising such compounds.

Described herein are uses of compounds described herein or a pharmaceutically acceptable salt thereof or pharmaceutical composition comprising such compounds as modulator of PFKFB3 and/or PFKFB4 activity.

Described herein are methods of modulating the activity of PFKFB3 and/or PFKFB4 in cell, comprising contacting the cell with compounds described herein or a pharmaceutically acceptable salt thereof or pharmaceutical composition comprising such compounds.

Described herein are uses of compounds described herein or a pharmaceutically acceptable salt thereof or pharmaceutical composition comprising such compounds for the treatment or prophylaxis of diseases or conditions for which glycolysis inhibition has beneficial effect.

Described herein are uses of compounds described herein or a pharmaceutically acceptable salt thereof or pharmaceutical composition comprising such compounds for the treatment or prophylaxis of diseases or conditions for which inhibition of kinase activity of PFKFB3 has beneficial effect.

Described herein are methods of treatment or prophylaxis of cancer, a neurodegenerative disease, an autoimmune disease, an inflammatory disorder, multiple sclerosis, a metabolic disease, or methods of inhibition of angiogenesis comprising administering to a subject in need thereof compounds described herein or a pharmaceutically acceptable salt thereof or pharmaceutical composition comprising such compounds.

Described herein are methods of treatment or prophylaxis of cancer comprising administering to a subject in need thereof compounds described herein or a pharmaceutically acceptable salt thereof or pharmaceutical composition comprising such compounds. Described herein are methods of treatment of cancer comprising administering to a subject in need thereof compounds described herein or a pharmaceutically acceptable salt thereof or pharmaceutical composition comprising such compounds.

Described herein are methods of prophylaxis of cancer comprising administering to a subject in need thereof compounds described herein or a pharmaceutically acceptable salt thereof or pharmaceutical composition comprising such compounds. In some embodiments, the cancer is a solid tumor cancer, such as kidney cancer, colon cancer, pancreatic cancer, lung cancer, breast cancer or liver cancer. In some embodiments, the cancer is a hematological cancer such as lymphoma, leukemia or myeloma.

Described herein are methods of inhibition of angiogenesis comprising administering to a subject in need thereof compounds described herein or a pharmaceutically acceptable salt thereof or pharmaceutical composition comprising such compounds. Described herein are methods of inhibition of angiogenesis comprising administering to a subject in need thereof compounds described herein or a pharmaceutically acceptable salt thereof or pharmaceutical composition comprising such compounds.

Described herein methods of treatment or prophylaxis of multiple sclerosis comprising administering to a subject in need thereof compounds described herein or a pharmaceutically acceptable salt thereof or pharmaceutical composition comprising such compounds. Described here methods of treatment of multiple sclerosis comprising administering to a subject in need thereof compounds described herein or a pharmaceutically acceptable salt thereof or pharmaceutical composition comprising such compounds. Described here methods of prophylaxis of multiple sclerosis comprising administering to a subject in need thereof compounds described herein or a pharmaceutically acceptable salt thereof or pharmaceutical composition comprising such compounds.

Described herein are methods of treatment or prophylaxis of a neurodegenerative disease comprising administering to a subject in need thereof compounds described herein or a pharmaceutically acceptable salt thereof or pharmaceutical composition comprising such compounds. Described herein are methods of treatment of a neurodegenerative disease comprising administering to a subject in need thereof compounds described herein or a pharmaceutically acceptable salt thereof or pharmaceutical composition comprising such compounds. Described herein are methods of prophylaxis of a neurodegenerative disease comprising administering to a subject in need thereof compounds described herein or a pharmaceutically acceptable salt thereof or pharmaceutical composition comprising such compounds. In some embodiments, the neurodegenerative disease is selected from Alzheimer's disease (including late onset), amyotrophic lateral sclerosis, stroke, Huntington's disease, and Parkinson's disease.

Described herein are methods of treatment or prophylaxis of an autoimmune disease comprising administering to a subject in need thereof compounds described herein or a pharmaceutically acceptable salt thereof or pharmaceutical composition comprising such compounds. Described herein are methods of treatment of an autoimmune disease comprising administering to a subject in need thereof compounds described herein or a pharmaceutically acceptable salt thereof or pharmaceutical composition comprising such compounds. Described herein are methods of prophylaxis of an autoimmune disease comprising administering to a subject in need thereof compounds described herein or a pharmaceutically acceptable salt thereof or pharmaceutical composition comprising such compounds. In some embodiments, the autoimmune disease is selected from celiac disease, psoriasis, systemic lupus erythematosus, scleroderma, graft-versus-host disease, or transplanted organ rejection.

Described herein are methods of treatment or prophylaxis of an inflammatory disorder comprising administering to a subject in need thereof compounds described herein or a pharmaceutically acceptable salt thereof or pharmaceutical composition comprising such compounds. Described herein are methods of treatment of an inflammatory disorder comprising administering to a subject in need thereof compounds described herein or a pharmaceutically acceptable salt thereof or pharmaceutical composition comprising such compounds. Described herein are methods of prophylaxis of an inflammatory disorder comprising administering to a subject in need thereof compounds described herein or a pharmaceutically acceptable salt thereof or pharmaceutical composition comprising such compounds. In some embodiments, the inflammatory disorder is selected from arthritis or inflammatory bowel disease.

Described herein are methods of treatment or prophylaxis of a viral disease, including but not limited to influenza comprising administering to a subject in need thereof compounds described herein or a pharmaceutically acceptable salt thereof or pharmaceutical composition comprising such compounds. Described herein are methods of treatment of a viral disease, including but not limited to influenza disease comprising administering to a subject in need thereof compounds described herein or a pharmaceutically acceptable salt thereof or pharmaceutical composition comprising such compounds. Described herein are methods of prophylaxis of viral disease, including but not limited to influenza comprising administering to a subject in need thereof compounds described herein or a pharmaceutically acceptable salt thereof or pharmaceutical composition comprising such compounds.

Described herein are methods of treatment or prophylaxis of metabolic diseases comprising administering to a subject in need thereof compounds described herein or a pharmaceutically acceptable salt thereof or pharmaceutical composition comprising such compounds. Described herein are methods of treatment of metabolic diseases comprising administering to a subject in need thereof compounds described herein or a pharmaceutically acceptable salt thereof or pharmaceutical composition comprising such compounds. Described herein are methods of prophylaxis of metabolic diseases comprising administering to a subject in need thereof compounds described herein or a pharmaceutically acceptable salt thereof or pharmaceutical composition comprising such compounds. In some embodiments, the metabolic disease selected from, glucose metabolism disorder, hyperlactatemia.

Described herein are methods for neuroprotection comprising administering to a subject in need thereof compounds described herein or a pharmaceutically acceptable salt thereof or pharmaceutical composition comprising such compounds.

Methods for treating any of the diseases or conditions described herein in a mammal in need of such treatment, involves administration of pharmaceutical compositions that include at least one compound described herein or a pharmaceutically acceptable salt thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 1 day, 2 days, 3 days, 4 days, 5 days, 6 days, 7 days, 10 days, 12 days, 15 days, 20 days, 28 days, 60 days, 80 days or more than 80 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment can be in the range of 0.01 mg-5000 mg per day. In one aspect, doses employed for adult human treatment are from about 1 mg to about 2 mg, from about 2 mg to about 5 mg, from about 5 mg to about 7 mg, from about 7 mg to about 10 mg, from about 10 mg to about 25 mg, from about 25 mg to about 50 mg, from about 50 mg to about 75 mg, from about 75 mg to about 100 mg, from about 100 mg to about 200 mg, from about 200 mg to about 500 mg, from about 500 mg to about 750 mg, from about 750 mg to about 1000 mg per day, from about 1000 mg to about 2000 mg per day, from about 2000 mg to about 3000 mg per day, from about 3000 mg to about 4000 mg, from about 4000 mg to about 5000 mg per day per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In other embodiments, the daily dosages appropriate for the compound described herein, or a pharmaceutically acceptable salt thereof, are (mg/kg) from about 0.01 to about 0.05, from about 0.05 to about 0.1, from about 0.1 to about 0.5, from about 0.5 to about 1, from about 1 to about 5, from about 5 to about 10, from about 10 to about 20, from about 20 to about 30, from about 30 to about 40, from about 40 to about 50, from about 50 to about 75, from about 75 to about 100, from about 100 to about 150, from about 150 to about 200, from about 200 to about 300 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies and clinical trials are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is about every 6 hours; (iii) the compound is administered to the mammal about every 8 hours; (iv) the compound is administered to the mammal about every 12 hours; (v) the compound is administered to the mammal about every 24 hours; (vi) the compound is administered to the mammal about every 36 hours, (vii) the compound is administered to the mammal about every 48 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from about 1 day to about 1 year.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In any case, regardless of the disease, disorder or condition being treated, the overall benefit experienced by the patient may be additive of the two or more therapeutic agents or the patient may experience a synergistic benefit. In some embodiments, such addictiveness can be related to at least one or more compounds, drugs or combinations described in or referred to in this application.

In some embodiments, the methods or uses described in this application comprise the additional step of co-administering to a patient a second therapeutic agent or combination of such agents or other therapies. If related to cancer additional therapies can include, for example: radiation therapy, surgery or administering additional therapeutic agent. In some embodiments the compound or composition of this invention may be administered together with additional therapeutic agent. It may be administered as a part of composition or any other single dosage form or separately. The additional therapeutic agent may be administered before, at the same time with, or after the administration of a compound or composition of one aspect of this invention. The administration of a composition of one aspect of this invention, comprising both a compound of one aspect of the invention and a second therapeutic agent, to a patient can go together with the separate administration of that same therapeutic agent, any other second therapeutic agent or any compound of one aspect of this invention to said patient at the same or another time during a course of treatment.

In some embodiments, the pharmaceutical composition described herein comprises at least one or more of the anti-cancer drugs known in the art or some of these drugs in any combinations, for example but not limited to the anti-cancer drugs approved by relevant regulatory agency as a therapy in cancer as FDA in US, European Medicines Agency in EU, CFDA in China and similar in other countries. The list of such drugs are available for example at web site of National Cancer Institute (e.g. https://www.cancer.gov/about-cancer/treatment/drugs), anti-cancer drug drug candidates currently in preclinical or clinical trials being tested in cancer, the list of such drugs are available for example in such websites as clinicaltrials.gov, www.clinicaltrialsregister.eu and commercial databases such as www.medtrack.com. In some embodiments, the pharmaceutical composition described herein comprises at least one or more of the anti-cancer drugs in any combinations from the list below: Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Adriamycin (Doxorubicin Hydrochloride), Adrucil (Fluorouracil), Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Bendamustine Hydrochloride, BEP, Bevacizumab, Bexarotene, Bexxar (Tositumomab and 1 131 Iodine Tositumomab), Bleomycin, Bortezomib, Bosulif (Bosutinib), Bosutinib, Brentuximab Vedotin, Cabazitaxel, Cabozantinib-S-Malate, CAF, Campath (Alemtuzumab), Camptosar (Irinotecan, ydrochloride), Capecitabine, CAPOX, Carboplatin, CARBOPLATIN-TAXOL, Carfilzomib, CeeNU (Lomustine), Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, Cosmegen (Dactinomycin), Crizotinib, CVP (COP), Cyclophosphamide, Cyfos (Ifosfamide), Cytarabine, Cytarabine, Liposomal, Cytosar-U (Cytarabine), Cytoxan (Cyclophosphamide), Dacarbazine, Dacogen, (Decitabine), Dactinomycin, Dasatinib, Daunorubicin Hydrochloride, Decitabine, Degarelix, Denileukin, iftitox, Denosumab, DepoCyt (Liposomal Cytarabine), DepoFoam (Liposomal Cytarabine), Dexrazoxane hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRINOX, FOLFOX, Folotyn (Pralatrexate), FU-LV, Fulvestrant, Gardasil (Recombinant HPV Quadrivalent Vaccine), Gefitinib, Gemcitabine Hydrochloride, GEMCITABINE-CISPLATIN, Gemtuzumab Ozogamicin, Gemzar (Gemcitabine, ydrochloride), Gleevec (Imatinib Mesylate), Glucarpidase, Halaven (Eribulin Mesylate), Herceptin (Trastuzumab), HPV Bivalent Vaccine, Recombinant, HPV Quadrivalent Vaccine (Recombinant), Hycamtin (Topotecan Hydrochloride), Ibritumomab Tiuxetan, ICE, Iclusig (Ponatinib Hydrochloride), Ifex (Ifosfamide), Ifosfamide, Ifosfamidum (Ifosfamide), Imatinib Mesylate, Imiquimod, Inlyta (Axitinib), Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic (Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Marqibo (Vincristine Sulfate Liposome), Matulane (Procarbazine Hydrochloride), Mechlorethamine Hydrochloride, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine hydrochloride), Mutamycin (Mitomycin C), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nolvadex (Tamoxifen Citrate), Nplate (Romiplostim), Ofatumumab, Omacetaxine, Mepesuccinate, Oncaspar (Pegaspargase), Ontak (Denileukin Diftitox), Oxaliplatin, Paclitaxel, Paclitaxel Albumin-stabilized Nanoparticle Formulation, Palifermin, Palonosetron Hydrochloride, Panitumumab, Paraplat (Carboplatin), Paraplatin (Carboplatin), Pazopanib Hydrochloride, Pegaspargase, Pemetrexed Disodium, Perjeta (Pertuzumab), Pertuzumab, Platinol (Cisplatin), Platinol-AQ (Cisplatin), Plerixafor, Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Raloxifene hydrochloride, Rasburicase, R-CHOP, R-CVP, Recombinant HPV Bivalent Vaccine, Recombinant HPV, Quadrivalent Vaccine, Regorafenib, Revlimid (Lenalidomide), Rheumatrex (Methotrexate), Rituxan (Rituximab), Romidepsin, Romiplostim, Rubidomycin (Daunorubicin Hydrochloride), Ruxolitinib Phosphate, Sclerosol Intrapleural Aerosol (Talc), Sipuleucel-T, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Synovir (Thalidomide), Synribo (Omacetaxine Mepesuccinate), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and I 131 Iodine Tositumomab, Totect (Dexrazoxane Hydrochloride), Trastuzumab, Treanda (Bendamustine Hydrochloride), Trisenox (Arsenic Trioxide), Tykerb (Lapatinib Ditosylate), Vandetanib, VAMP, Vectibix (Panitumumab), VelP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELOX, Xgeva (Denosumab), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoledronic Acid, Zolinza (Vorinostat), Zometa (Zoledronic Acid), or Zytiga (Abiraterone Acetate). In some embodiments of this invention, the additional therapeutic agents and combinations described in this paragraph, or described in or refered to in this application can be used as separate, multiple dosage forms in addition to the compound and combination according one aspect of this invention,

Described herein are also methods of treating or preventing an age-related disease or disorder or other anti-aging treatment comprising administering to a subject in need thereof a PFKFB3 inhibitor or the modulator at least one of Indirect Targets. One of the methods to test the efficacy of such anti-aging treatment is to check biomarkers related to aging and mortality risks.

In some embodiments, selected biomarkers related to aging and mortality risks can be used to evaluate if the subject is regarded to be aged. In some embodiments, subject is said to be “aged” or “old” when blood of such subject has a concentration of its elements that falls within the range of concentrations related to the moderate or high risk of mortality described in the available sources regarding the correlation of corresponding parameters with mortality, e.g. as described in https://www.ncbi.nlm.nih.gov/pmc/articles/PMC5611985/ or on the website http://mortalitypredictors.org and the publications cited there on blood predictors of mortality or in any other source.

In some embodiments, the compounds and compositions of this disclosure are useful for changing selected biomarkers related to aging or mortality or morbidity risks, including but not limited to described in this disclosure into a younger state and thus reducing the risks of mortality and/or morbidity.

In some embodiments, biomarkers mentioned in this description, could be used to identify the biological age of a subject and/or to verify whether a treated subject responds to treatment (e.g. if one or more of the biomarkers change to a level characteristic of a younger age or delay in changing into the level characteristic of older age).

In some embodiments, biomarkers of aging, biological age metrics, chronological age metrics, mortality biomarkers, morbidity biomarkers, health declines, biomarkers of stress resistance, biomarkers of resilience, frailty index, frailty biomarkers, biomarkers of particular age related diseases and conditions can be used to verify whether a treated subject responds to treatment (e.g. if one or more of the biomarkers change to a level characteristic of a younger age or delay in changing into the level characteristic of older age).

Every web link cited in this application, in case of inaccessibility as a rule can be retrieved via https://web.archive.org or similar internet archive services.

In some embodiments, the one or two or more biomarkers, as referred to in reference to biomarkers characteristic of an aged subject, (with associated measurement units in plasma) are selected from the group: Glucose, serum (mg/dL); Creatinine (mg/dL); Lactate dehydrogenase LDH (U/L); Uric acid (mg/dL); Blood lead (ug/dL); Homocysteine(umol/L); Vitamin A (ug/dL); Fasting Glucose (mg/dL); GGT: SI (U/L); Total cholesterol (mg/dL); Vitamin E (ug/dL); Chloride: SI (mmol/L); AST: SI (U/L); Sodium: SI (mmol/L); PCB180 (ng/g); Cholesterol (mg/dL); PCB170 (ng/g); Alkaline phosphatase(U/L); PCB180 Lipid Adjusted; Oxychlordane Lipid Adjusted; 3,3′,4,4′,5,5′-hexachlorobiphenyl (hxcb) (fg/g); PCB74 (ng/g); PCB170 Lipid Adjusted; Triglycerides (mg/dL); PCB153 (ng/g); Oxychlordane (ng/g); PCB74 Lipid Adjusted; Monocyte percent (%); Ferritin (ng/mL); 3,3′,4,4′,5,5′-hexachlorobiphenyl (hxcb) Lipid Adjusted; 2,3,4,7,8-Pentachlorodibenzofuran (pncdf) (fg/g); Methylmalonic acid (umol/L); PCB153 Lipid Adjusted; PCB187 (ng/g); 2,3,4,7,8-Pentachlorodibenzofuran (pncdf) Lipid Adjusted; PCB156 (ng/g); White blood cell count: SI; PCB187 Lipid Adjusted; 1,2,3,6,7,8-Hexachlorodibenzo-p-dioxin (hxcdd)(fg/g); Trans-nonachlor Lipid Adjusted; PCB138 (ng/g); 4-pyridoxic acid (nmol/L); Potassium: SI (mmol/L); Trans-nonachlor (ng/g); 1,2,3,6,7,8-Hexachlorodibenzo-p-dioxin (hxcdd) Lipid Adjusted; PCB138 Lipid Adjusted; PCB118 (ng/g); PCB156 Lipid Adjusted; PCB118 Lipid Adjusted; Mean cell volume (fL); PCB146 (ng/g); Blood cadmium (ug/L); Two hour oral glucose tolerance (OGTT) (mg/dL); Folate, serum (ng/mL); PCB194 Lipid Adjusted; PCB194 (ng/g); Hematocrit (%); 1,2,3,4,7,8-Hexachlorodibenzofuran (hcxdf) (fg/g); Perfluorohexane sulfonic acid (ug/L); RBC folate (nmol/L); PCB99 (ng/g); p,p′-DDE (ng/g); p,p′-DDE Lipid Adjusted; Total Serum Foalte (nmol/L); PCB146 Lipid Adjusted; PCB196 Lipid Adjusted; PCB196 (ng/g); 1,2,3,4,6,7,8,9-Octachlorodibenzo-p-dioxin (ocdd) (fg/g); PCB183 (ng/g); Perfluorooctane sulfonic acid; 3,3′,4,4′,5-Pentachlorobiphenyl (pncb) (fg/g); trans-lycopene(ug/dL); 1,2,3,7,8-Pentachlorodibenzo-p-dioxin (pncdd) (fg/g); 1,2,3,4,6,7,8-Heptachlororodibenzo-p-dioxin (hpcdd) (fg/g); 3,3′,4,4′,5-Pentachlorobiphenyl (pncb) Lipid Adjusted; 1,2,3,4,7,8-Hexachlorodibenzofuran (hcxdf) Lipid Adjusted; 1,2,3,6,7,8-Hexachlorodibenzofuran (hxcdf) (fg/g); PCB99 Lipid Adjusted; Triiodothyronine (T3), free (pg/mL); 1,2,3,4,6,7,8,9-Octachlorodibenzo-p-dioxin (ocdd) Lipid Adjusted; a-Tocopherol(ug/dL); Blood o-Xylene Result; Beta-hexachlorocyclohexane Lipid Adjusted; Plasma glucose: SI(mmol/L); 1,2,3,7,8-Pentachlorodibenzo-p-dioxin (pncdd) Lipid Adjusted; Parathyroid Hormone(Elecys method) pg/mL; Beta-hexachloro-cyclohexane (ng/g); 1,2,3,4,6,7,8-Heptachlororodibenzo-p-dioxin (hpcdd) Lipid Adjusted; PCB105 (ng/g); PCB177 (ng/g); Hemoglobin (g/dL); Heptachlor Epoxide (ng/g); Perfluorooctanoic acid; Heptachlor Epoxide Lipid Adjusted; 1,2,3,6,7,8-Hexachlorodibenzofuran (hxcdf) Lipid Adjusted; PCB183 Lipid Adjusted; 2,3,7,8-Tetrachlorodienzo-p-dioxin (tcdd) (fg/g); Vitamin B12, serum (pg/mL); cis-b-carotene(ug/dL); Cotinine (ng/mL); 1,2,3,7,8,9-Hexachlorodibenzo-p-dioxin (hxcdd) (fg/g); Triglyceride (mg/dL); p,p′-DDT (ng/g); Triiodothyronine (T3), total (ng/dL); PCB105 Lipid Adjusted; 1,2,3,4,7,8-Hexachlorodibenzo-p-dioxin (hxcdd)(fg/g); Mean cell hemoglobin (pg); Dieldrin (ng/g); Folate, RBC (ng/mL RBC); Aldrin; trans-b-carotene(ug/dL); Eosinophils percent (%); Endrin; Bone alkaline phosphotase (ug/L); PCB199 Lipid Adjusted; 1,2,3,4,7,8-Hexachlorodibenzo-p-dioxin (hxcdd) Lipid Adjusted; 1,2,3,7,8,9-Hexachlorodibenzo-p-dioxin (hxcdd) Lipid Adjusted; Dieldrin Lipid Adjusted; p,p′-DDT Lipid Adjusted; Segmented neutrophils percent (%); 2,3,7,8-Tetrachlorodienzo-p-dioxin (tcdd) Lipid Adjusted; Retinyl stearate (ug/dL); PCB151 (ng/g); PCB149 (ng/g); Perfluorononanoic acid (ug/L); PCB177 Lipid Adjusted; PCB178 Lipid Adjusted; PCB209 (ng/g); PCB178 (ng/g); 5-Methyl THF(nmol/L); PCB209 Lipid Adjusted (ng/g); C-peptide (nmol/L) in SI units; Platelet count (%) SI; Blood Bromodichloromethane Result; Total iron binding capacity (ug/dL); Red cell distribution width (%); Blood Chloroform Result; Glycidamide (pmoL/G Hb); Testosterone total (ng/dL); Hexachlorobenzene (ng/g); Apolipoprotein (B) (mg/dL); ALT: SI (U/L); 25-hydroxyvitamin D2+D3; PCB206 Lipid Adjusted; Follicle stimulating hormone (mIU/mL); Basophils percent (%); 2-(N-Methyl-perfluorooctane sulfonamido) acetic acid (ug/L); Vitamin B6 (Pyridoxal 5′-phosphate) test results (nmol/L); Pyridoxal 5′-phosphate (nmol/L); total Lycopene(ug/dL); Blood Methyl t-Butyl Ether (MTBE) Result; Helicobacter pylori (ISR); PCB167 Lipid Adjusted; Mirex (ng/g); Luteinizing hormone (mIU/mL); Blood manganese (ug/L); Mean cell hemoglobin concentration (g/dL); PCB128 (ng/g); a-Cryptoxanthin(ug/dL); Thyroxine, free (ng/dL); cis-Lycopene(ug/dL); Thyroid stimulating hormone (uIU/mL); PCB172 Lipid Adjusted; Blood mercury, total (ug/L); Inorganic mercury, blood (ug/L); 2,2′,4,4′,5,5′-hexabromobiphenyl (pg/g); Vitamin C (mg/dL); Blood m-/p-Xylene Result; PCB167 (ng/g); Mercury, methyl (ug/L); Combined Lutein/zeaxanthin(ug/dL); 2,2′,4,4′,5,6′-hexabromodiphenyl ether (pg/g); Folic acid, serum (nmol/L); Acrylamide (pmoL/G Hb); 2,2′,4,4′,5,5′-hexabromobiphenyl lipid adjusted (ng/g); 2,3,4,6,7,8, -Hexchlorodibenzofuran (hxcdf) (fg/g); total b-Carotene(ug/dL); 25-hydroxyvitamin D3(nmol/L); Perfluoroundecanoic acid (ug/L); Protoporphyrin (ug/dL RBC); PCB206 (ng/g); PCB157 Lipid Adjusted; Phytofluene(ug/dL); Aldrin Lipid Adjusted; epi-25-hydroxyvitamin D3 (nmol/L); PCB172 (ng/g); PCB66 (ng/g); Endrin Lipid Adjusted; a-carotene(ug/dL); Trans 9, trans 12-octadienoic acid (uM); PCB28 (ng/g); Pefluorodecanoic acid (ug/L); Lymphocyte percent (%); Thyroid stimulating hormone (IU/L); 1,2,3,4,6,7,8-Heptachlorodibenzofuran (hpcdf) (fg/g); Hexachlorobenzene Lipid Adjusted; Mirex Lipid Adjusted; Total dust weight (mg); Insulin: SI(pmol/L); Sieved dust weight (mg); Serum Selenium (ug/L); Lutein(ug/dL); Blood Nitromethane (pg/mL); Gamma-hexachlorocyclohexane Lipid Adjusted; Retinyl palmitate (ug/dL); Trans 9-octadecenoic acid (uM); 1,2,3,7,8,9-Hexachlorodibenzofuran (hxcdf) (fg/g); 1,2,3,4,7,8,9-Heptachlorodibenzofuran (Hpcdf) (fg/g); PCB87 (ng/g); and Red cell count SI. In some embodiments, the two or more biomarkers are selected from the group: Glucose, serum (mg/dl); Creatinine (mg/dl); Lactate dehydrogenase LDH (U/L); Uric acid (mg/dl); Blood lead (ug/dl); Homocysteine(umol/L); Vitamin A (ug/dl); Fasting Glucose (mg/dl); GGT: SI (U/L); Total cholesterol (mg/dl); Vitamin E (ug/dl); Chloride: SI (mmol/L); AST: SI (U/L); Sodium: SI (mmol/L); PCB180 (ng/g); Cholesterol (mg/dl); PCB170 (ng/g); Alkaline phosphatase(U/L) and glycohemoglobin. In some embodiments, biomarkers characteristic of aging are selected from: glucose serum, glycohemoglobin, creatine, lactate dehydrogenase, uric acid, blood lead, homocysteine, vitamin A, fasting glucose, gamma glutamyltransferase (GGT), total cholesterol, Vitamin E, chloride, aspartate aminotransferase (AST), sodium, and 2,2′,3,4,4′,5,5′-heptachlorobiphenyl (PCB180). In some embodiments, biomarkers characteristic of aging are selected from: glucose serum, glycohemoglobin, creatine, lactate dehydrogenase, uric acid, blood lead, homocysteine, vitamin A, fasting glucose, gamma glutamyltransferase (GGT), and total cholesterol. In some embodiments, biomarkers characteristic of aging are selected from: glucose serum, glycohemoglobin, creatine, lactate dehydrogenase, uric acid, melatonin and blood lead.

TABLE 3
Non-limiting list of selected biomarkers related to mortality risks
Name

Red blood cell distribution width
Mean reticulocytes volume
Neutrophil number
Alpha-1-acid glycoprotein
White blood cell count
Hemoglobin
Red blood cell count
Monocyte count
Basophil count
Neutrophils percentage
Albumin, serum/plasma
Lymphocyte percentage
Hematocrit
Leukocyte telomere length
Mean sphered cells volume
Very-low-density lipoprotein
Insulin-like growth factor 1
Mean corpuscular hemoglobin
Mean corpuscular volume
Citrate
trans-lycopene
Monocyte percentage
Platelet count
Reticulocytes count
Lymphocyte count
Platelet distribution width
Plateletcrit
Immature reticulocytes fraction
Reticulocytes, high light scatter, percentage
Reticulocytes, high light scatter, number
Reticulocytes percentage
Soluble CD14
Eosinophils percentage
25-hydroxyvitamin D
Adiponectin
Ascorbic acid
Brain natriuretic peptide
C-reactive protein
Cardiac troponin I
Estimated glomerular filtration rate
Fibroblast growth factor-23
Gamma-glutamyltransferase
Glucose
Growth differentiation factor 15
H-FABP
Homeostatic model assessment of insulin resistance
Homocysteine
Lipoprotein-associated phospholipase A2 activity
N-terminal atrial natriuretic peptide
SUA
Type I collagen degradation
Vitamin A
High-density lipoprotein cholesterol
Klotho
Leptin
Club (a.k.a. Clara) cell secretory protein
Antinuclear autoantibodies
Soluble ST2
Alanine transaminase
Alkaline phosphatase
Alpha-1-antichymotrypsin
Angiopoietin-2
ApoB/ApoA1 ratio
Asymmetric dimethylarginine
C-reactive protein, high-sensitivity
Cardiac troponin T, high-sensitivity
Cholesterol
Creatinine
Cystatin C
Fibrinogen
Glycated hemoglobin
Growth hormone
Homoarginine
Insulin-like growth factor binding protein 1
Interleukin-6
Low-density lipoprotein cholesterol
Lycopene
Mitochondrial DNA copy number
N-terminal pro-brain natriuretic peptide
Neutrophil gelatinase-associated lipocalinin
Osteocalcin
Osteoprotegerin
Phosphorus
Testosterone
Triglycerides
Tumor necrosis factor alpha
Uric acid
alpha-carotene
beta-trace protein
β2-microglobulin
Anion gap, serum albumin adjusted
CD4:CD8 ratio
CD8 cells
Carboxyl-terminal telopeptide of collagen type I
Plasma viscosity
Insulin-like growth factor binding protein 2
Peroxiredoxin 4
Stromal cell derived factor
Carotenoids
Oxygenated carotenoids
Urea
sj/β-TREC ratio
Insulin-like growth factor binding protein 3
Proinsulin
Factor VIIc
IgA to tissue transglutaminase
Bilirubin
Mean platelet volume
Galectin-3
Interleukin-8
Loss of Y chromosome
Soluble tumour necrosis factor receptor 1
Symmetrical dimethyl arginine
T cells
Thyroxine
Cell-free DNA concentration
beta-cryptoxanthin
Basophil percentage
Interleukin-10
Apolipoprotein A-1
Amino-terminal propeptide of type I collagen
Estradiol-to-sex hormone binding globulin ratio
Neutrophilia
Butyrylcholinesterase activity
Reticulocytes number
Parathyroid hormone
Follicle stimulating hormone
Interleukin 1-beta
17beta-E(2)
Amino-terminal peptide of procollagen type III

In some embodiments this invention is a method, including but not limited to the method of anti-aging treatment or neuroprotection, comprising administering by the subject at least one of the compositions, molecules or other agents described in this disclosure, including but not limited to PFKFB3 inhibitors in therapeutically effective amount. In some embodiments this invention is a method, comprising administering to the subject an effective amount of molecule selected from the group: monoclonal or polyclonal antibody, protein, aptamer, peptide, polymer, virus or small molecule or any other PFKFB3 inhibitor. In some embodiments this invention is a method of treatment, wherein an molecule for administration is PFKFB3 inhibitor described in this application or is its analog, prodrug or derivative.

In some embodiments this invention is a method of treatment, including but not limited to anti-aging treatment or treatment of neurodegenerative disease, comprising step of administering by the subject of agent, deactivating or binding or inhibiting or degrading a PFKFB3 or deactivating or binding or inhibiting or degrading or activating at least one of Indirect Targets, what has anti-aging or neuroprotective effect, including but not limited at least one agent described in this disclosure, including but not limited to agent selected from the group: monoclonal antibody, polyclonal antibody, fAb, protein, aptamer, peptide, polymer, virus or small molecule or at least one of the agents selected from the PFKFB3 inhibitors described in this application or is its analog.

The dosage levels and mode of administration can be dependent on a variety of factors such as the treatment used, the active agent, the context of use (e.g., the patient to be treated), and the like. Optimization of modes of administration, dosage levels, and adjustment of protocols, including monitoring systems to assess effectiveness are routine matters well within ordinary skill. In some embodiments, optimization of modes of administration, dosage levels, and adjustment of protocols etc. are designed to keep PFKFB3 concentration in negligible amount most of the time, or for at about 1 month, or from 1 months to 6 months, or from 6 months to 12 months, or from 12 months to 24 months, or from 24 months to 48 months, or for up to 5 years, or for up to 10 years, or from about 1 month to about 10 years, or for more then 10 years, or for as long as possible, or for lifelong or for other period defined by doctor or patient.

Further discussion of optimization of dosage and treatment regimens can be found in Benet et al., in Goodman & Gilman's The Pharmacological Basis of Therapeutics, Ninth Edition, Hardman et al., Eds., McGraw-Hill, New York, (1996), Chapter 1, pp. 3-27, and L. A. Bauer, in Pharmacotherapy, A Pathophysiologic Approach, Fourth Edition, DiPiro et al., Eds., Appleton & Lange, Stamford, Conn., (1999), Chapter 3, pp. 21-43, and the references cited therein, to which the reader is referred.

In some embodiments, Biological age or chronological age determined with the use of data from blood characterizes the health status or biological age or chronological age of the subject. In some embodiments, the blood based biological age determination approach is described in prior art, including but not limited to any of the following publications, https://www.ncbi.nlm.nih.gov/pmc/articles/PMC5514388/. https://www.ncbi.nlm.nih.gov/pmc/articles/PMC4931851/, https://www.ncbi.nlm.nih.gov/pmc/articles/PMC5514388/ and corresponding references related to blood based biological age determination.

In some embodiments, the biological age is understood as the distance measured along a continuous trajectory consisting of distinct phases, each corresponding to subsequent human life stages as described in more details in “Quantitative Characterization of Biological Age and Frailty Based on Locomotor Activity Records”, Pyrkov et al., 2017) https://www.biorxiv.org/content/biorxiv/early/2017/09/09/186569.full.pdf

In some embodiments, the biological age is understood in the following context. The confinement of the aging dynamics of the physiological variables to the low-dimensional manifold representing the aging trajectory is a hallmark of criticality. It has been long suggested that the regulatory systems governing the dynamics of the organism state vector operate near the order-disorder boundary. The biological age is then the order parameter, associated with the organism development and aging, satisfies a stochastic Langevin equation in an unstable effective potential characterize by the single number, the underlying regulatory network stiffness. The number describes the organism state deviations from the youthful state and has the meaning of the number of regulatory abnormalities accumulated over the course of the organism life history, is associated with the decreased resilience and amplified risks of morbidities and death. We suggested that the stochastic biological age dynamics is the mechanistic origin of Gompertz mortality law. The exponential acceleration of the morbidity and mortality rates is the characteristic feature of aging in adult individuals or older. The reduction of the aging dynamics to essentially a one-dimensional manifold, a consequence of the criticality of the underlying regulatory network, means that the distance traveled along the aging trajectory is thus a progress indicator of the process of aging and hence is a natural biomarker of age. The biological age acceleration, i.e., the difference between the biological age of an individual and average the biological age prediction in the sex- and the age-matched cohort of their peers, is elevated for patients with chronic diseases. It is a powerful predictor of all-cause mortality even after confounding by the standard Health Risks Assessment (HRA) variables such as age, sex, and smoking status.

In some embodiments, the biological age is understood as the biomarker or metric based on one or more several biomarkers predicting risks of morbidity and/or death in 8 years or later or in range of mortality rate doubling time or later.

In some embodiments, the aged subject is understood as a subject with high mortality risk in about 1 month, in about 3 months, in about 6 months, in about 1 year, from about 1 month to about 6 months, from about 1 month to about 1 year, from about 1 year to about 3 years, from about 3 years to about 5 years, from about 5 years to about 8 years, from about 5 years to about 10 years, in about 5 years, in about 10 years, in about 15 years. In some embodiments, high mortality risk is a risk of dying from age related condition or disease. In some embodiments, high mortality risk is all cause mortality risk. Non limiting examples of blood based biomarkers of mortality and its critical volumes are described in prior art, including but not limited to https://www.ncbi.nlm.nih.gov/pmc/articles/PMC4899173/, https://www.ncbi.nlm.nih.gov/pmc/articles/PMC4454670/, https://www.ncbi.nlm.nih.gov/pmc/articles/PMC5334528/, Maximus Peto, Carlos De la Guardia, Ksenia Winslow, Andrew Ho, Kristen Fortney, & Eric Morgen. “Mortalitypredictors.org, a manually curated database of published biomarkers of human all-cause mortality. Aging, 2017.

In some embodiments, this disclosure provides a method of anti-aging treatment of the subject, having one or two or more biomarkers characteristic of subject with high morbidity risk in about 1 month, in about 3 months, in about 6 months, in about 1 year, from about 1 month to about 6 months, from about 1 month to about 1 year, from about 1 year to about 3 years, from about 3 years to about 5 years, from about 5 years to about 8 years, from about 5 years to about 10 years, in about 5 years, in about 10 years, in about 15 years. In some embodiments, high morbidity risk is a risk of acquiring an age related condition or disease.

In some embodiments, this disclosure provides a method of anti-aging treatment of the subject, having one or two or more biomarkers characteristic of subject with age related condition or disease or high risk of such disease, including but not limited to type 2 diabetes, age-related cardiovascular diseases, including but not limited to ischemic heart disease and stroke, metabolic syndrome, COPD, Alzheimer's disease etc., including but not limited those mentioned in this disclosure or at least one of the aging related declines. In some embodiments subject is understood as aged and having an aging related decline in case the corresponding parameter of subject health or appearance is changed into elder state in comparison with the own parameter of the same subject or in comparison with the median volume of the same parameter in statistically meaningful number of people of same gender of 25 years old in HNAHES study or statistically meaningful number of random people of same gender of 25 years old, optionally same race and residents of the same country or region.

In some embodiments, high (mortality or morbidity or age related disease or age related condition) risk is more than 90%, more than 80%, more than 70%, more than 60%, more than 50%, more than 40%, more than 30%, more than 20%, more than 10%, more than 5%, more than 3%, more than 1%, more than 0.5%, more than 0.1%, more than 0.05%.

In some embodiments, PFKFB3 inhibitors described in this application or its structural or functional analogs or is agent comprising the part binding PFKFB3 at least 99% structurally similar, at least 95% structurally similar, at least 90% structurally similar, or at least 80% structurally similar, or at least 70% structurally similar to the part binding such protein of at least one of the PFKFB3 inhibitors described in this application.

There are a lot of elements of blood plasma changing with the age and metrics based on it which can be indicative for the chronological or biological age and/or health status of the person from such as proteins, metabolites etc. and there are many methods to calculate a biological or chronological age of the person whose plasma is used is known and new methods of calculation are constantly being introduced. Any of such methods to calculate a biological or chronological age of the person whose plasma is used can be used to define plasma as aged or made from the aged blood or comprising biomarkers of aged blood.

One of the ways to estimate age of the subject it is to describe what elements in what amount are contained in blood and compare it to the data regarding known concentration/amounts of those of such elements in blood plasma which amount changes with the age, known in the art. As a non-limiting example, some of such elements of plasma are shown in this application. These biomarkers can be measured and analyzed by the methods known in the art, as an example some biomarkers found in blood plasma of people of different age in The National Health and Nutrition Examination Survey (NHANES) (a program of studies designed to assess the health and nutritional status of adults and children in the United States). In some embodiments “Old” or “Aged” level of proteins or other plasma elements means the level of plasma proteins or other elements of plasma which concentration is changing with the age or their combination or metric based on such plasma proteins or other elements that corresponds to the median or average level of the of people aged at least 30, 35, 40, 45, 50, 55, 60, 65, 70,75,80, 85, 90 and elder is known in the art and can be measured by many methods known in the art and yet to be introduced.

There are many different ways and tools to measure expression amount of the particular proteins in blood plasma known in the art that can also be used for evaluation if the person whose blood is used is aged. The non-limiting examples of such technologies and tools are SOMAscan® Assay of Somalogic (http://somalogic.com), biomarker panels of Olink Proteomics (http://www.olink.com/), ELISA, multiplexes comprised of antibodies binding to the particular proteins e.g. Luminex technology (https://www.rndsystems.com/products/luminex-assays-and-high-performance-assays), mass spectrometry etc.

Though technologies to estimate the amounts of proteins are different and usually use the different units most of them can give the estimation on the relative amount of each protein in plasma and how this amount changes with the age, biological age and health status.

Described herein are uses of compounds described herein or a pharmaceutically acceptable salt thereof or pharmaceutical composition comprising such compounds as modulator of PFKFB3 activity.

Described herein are methods of modulating the activity of PFKFB3 in cell, comprising contacting the cell with compounds described herein or a pharmaceutically acceptable salt thereof or pharmaceutical composition comprising such compounds.

Described herein are uses of compounds described herein or a pharmaceutically acceptable salt thereof or pharmaceutical composition comprising such compounds for the treatment or prophylaxis of diseases or conditions for which glycolysis inhibition has beneficial effect.

Described herein are uses of compounds described herein or a pharmaceutically acceptable salt thereof or pharmaceutical composition comprising such compounds for the treatment or prophylaxis of diseases or conditions for which inhibition of kinase activity of PFKFB3 has beneficial effect.

Described herein are also methods of treatment or prophylaxis of a neurodegenerative disease, an multiple sclerosis, comprising administering to a subject in need thereof compounds described herein or a pharmaceutically acceptable salt thereof or pharmaceutical composition comprising such compounds.

Described herein also methods of treatment or prophylaxis of multiple sclerosis comprising administering to a subject in need thereof compounds described herein or a pharmaceutically acceptable salt thereof or pharmaceutical composition comprising such compounds. Described here methods of treatment of multiple sclerosis comprising administering to a subject in need thereof compounds described herein or a pharmaceutically acceptable salt thereof or pharmaceutical composition comprising such compounds.

Described here methods of prophylaxis of multiple sclerosis comprising administering to a subject in need thereof compounds described herein or a pharmaceutically acceptable salt thereof or pharmaceutical composition comprising such compounds.

Described herein are also methods of treatment or prophylaxis of a neurodegenerative disease comprising administering to a subject in need thereof compounds described herein or a pharmaceutically acceptable salt thereof or pharmaceutical composition comprising such compounds. Described herein are methods of treatment of a neurodegenerative disease comprising administering to a subject in need thereof compounds described herein or a pharmaceutically acceptable salt thereof or pharmaceutical composition comprising such compounds. Described herein are methods of prophylaxis of a neurodegenerative disease comprising administering to a subject in need thereof compounds described herein or a pharmaceutically acceptable salt thereof or pharmaceutical composition comprising such compounds. In some embodiments, the neurodegenerative disease is selected from Alzheimer's disease (including late onset), amyotrophic lateral sclerosis, stroke, Huntington's disease, and Parkinson's disease.

Described herein are also methods for neuroprotection comprising administering to a subject in need thereof compounds described herein or a pharmaceutically acceptable salt thereof or pharmaceutical composition comprising such compounds.

Methods for treating any of the diseases or conditions described herein in a mammal in need of such treatment, involves administration of pharmaceutical compositions that include at least one compound described herein or a pharmaceutically acceptable salt thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 1 day, 2 days, 3 days, 4 days, 5 days, 6 days, 7 days, 10 days, 12 days, 15 days, 20 days, 28 days, 60 days, 80 days or more than 80 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 1 day, from about 1 day to about 3 days, from about 3 days to about 1 week, from about 2 weeks to about 1 month, from about 1 month to about 2 months, from about 2 months to about 4 months, from about 4 months to about 6 months, from about 6 months to about 12 months, from about 12 months to about 18 months, from about 18 months to about 24 months, from about 2 years to about 5 years, more than 5 years, lifelong.

### EXAMPLES

### Chemical Abbreviations

0. The names of chemical compounds here are named according the IUPAC nomenclature.
HATU: 1-[bis(dimethylamino)methylene]-1H-1,2,3-triazolo[4,5-b]pyridinium 3-oxide hexafluorophosphate.
DMSO: dimethyl sulfoxide.
DMF: N,N-dimethylformamide.
DCC: N,N′-Dicyclohexylcarbodiimide
DMAP: 4-dimethylaminopyridine.
EDCI: N-(3-dimethylaminopropyl)-N′-ethylcarbodiimide hydrochloride.
DIPEA: N,N′-diisopropylethylamine.
HPLC: high-performance liquid chromatography.
TFA: trifluoroacetic acid.
LC/MS: liquid chromatography/mass spectrometry.
THF: tetrahydrofuran.
TBAF: tetra-n-butylammonium fluoride.
TMS: trimethylsilyl.
TMSN₃: trimethylsilyl azide.
dppf: 1,1′-bis(diphenylphosphino)ferrocene.
PdCl₂dppf: [1,1′-bis(diphenylphosphino)ferrocene]palladium(II) dichloride.
PdCl₂(PPh₃)₂: bis(triphenylphosphine)palladium(II) dichloride.
Pd(PPh₃)₄: tetrakis(triphenylphosphine)palladium(0).
Pd(dba)₂: Bis(dibenzylideneacetone)palladium(0)
RT: room temperature
dpp: 1,3-bis(diphenylphosphino)propane.
Sphos: 2-dicyclohexylphosphino-2′,6′-dimethoxybiphenyl.
NBS: N-bromosuccinimide.
AIBN: azobisisobutyronitrile.
mCPBA: 3-chloroperoxybenzoic acid.
Boc: tert-butoxycarbonyl, protecting group.
Fmoc: fluorenylmethyloxycarbonyl, protecting group.

1. The following examples are provided for illustrative purposes only and not to limit the scope of the claims provided herein. To avoid any doubts in interpretation of the wording in the following clauses from 1 to 278 below, a reference to “Example” of corresponding number means a reference to the part of the text in clauses from 1 to 278 below containing the word Example with corresponding number of example, but not the reference to the clause of corresponding number. For example, Clause 133 below contains the following wording: “2-(3-Methoxycarbonylbiphenyl-4-yl)-1,3-dioxo-2,3-dihydro-1H-isoindole-5-carboxylic acid methyl ester was prepared as described in synthetic procedure AD from 2-(3-methoxycarbonylbiphenyl-4-yl)-1,3-dioxo-2,3-dihydro-1H-isoindole-5-carboxylic acid (see Example 12) . . . ”. The mentioned “Example 12” is contained in clause 139 . . . Example 12: 2-(3-Methoxycarbonylbiphenyl-4-yl)-1,3-dioxo-2,3-dihydro-1H-isoindole-5-carboxylic acid

Synthetic Procedure A: Synthesis of Methyl Arylanthranilate from Methyl Bromoanthranilate

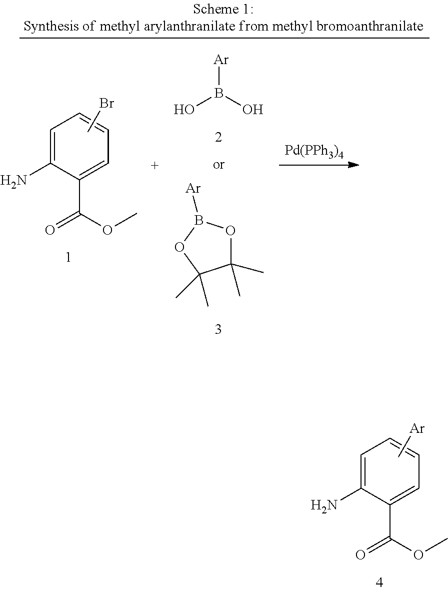

2. Methyl 4(or 5)-bromoanthranilate (Compound 1 in Scheme 1, 500 mg, 2.17 mmol) and arylboronic acid (Compound 2 on Scheme 1, 2.17 mmol) or arylboronic acid pinacol ester (Compound 3 in Scheme 1, 2.17 mmol) were dissolved in dioxane (13 mL) and Pd(PPh₃)₄ (250 mg, 0.217 mmol) and sodium carbonate (1.5M, 4.34 mL, 6.51 mmol) were added. The mixture was heated in a microwave reactor at 100° C. for 12 h. The cooled solution was partitioned between ethyl acetate and water, the organic layer separated and washed with brine and the solvents evaporated to afford a crude material. The residue was purified by chromatography (silica gel, hexane/EtOAc=100/0-40/60) to give the title compound methyl 4(or 5)-arylanthranilate (Compound 4 in Scheme 1, 71-90%).

Synthetic Procedure B: Synthesis of Arylanthranilic Acid

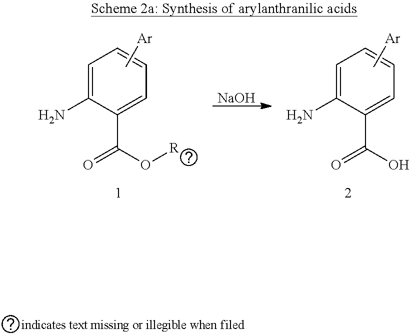

3. A solution of methyl arylanthranilate (Compound 1 in Scheme 2a, 1.75 mmol) in 1 M aqueous sodium hydroxide (6.9 mL, 6.9 mmol) and THF (3.5 mL) was heated at reflux overnight. The mixture was cooled to RT and concentrated almost to dryness. 6 M hydrochloric acid (0.1 mL) was added to the solution at 0° C. and the precipitate was collected by filtration, washed with water and dried to give arylanthranilic acid (Compound 2 in Scheme 2a, 63-84%) as a white solid.

Synthetic Procedure C: Synthesis of Methyl Pinacolboranylanthranilate

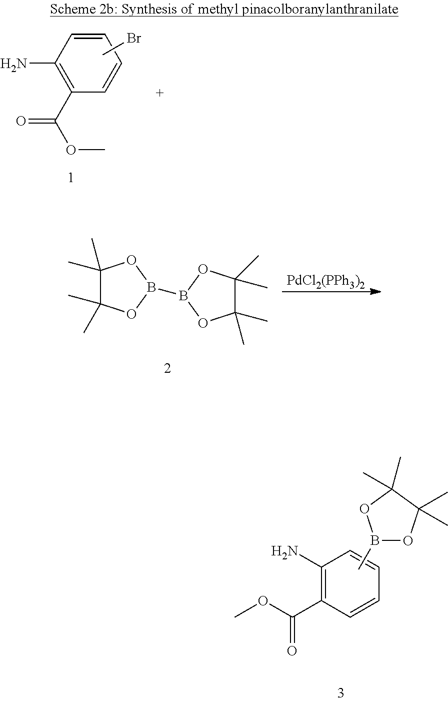

4. Synthesis of methyl 4-pinacolboranylanthranilate: Methyl 4-bromoanthranilate (Compound 1 in Scheme 2b, 0.230 g, 1 mmol) and bis(pinacolato)diboron (Compound 2 in Scheme 2b, 0.305 g, 1.2 mmol) were dissolved in anhydrous dioxane (15 mL) then PdCl₂(PPh₃)₂ (0.039 g, 0.055 mmol) was added followed by potassium acetate (0.294 g, 3 mmol). The mixture was stirred at 85° C. for 15 h. The solution was cooled and the solvent removed under reduced pressure. The residue was partitioned between ethyl acetate and water. The organic layer was washed with brine, dried and evaporated to give the title compound methyl 4-pinacolboranylanthranilate (Compound 3 in Scheme 2b, 0.263 g, 95% yield) which was used without further purification.

Synthesis of methyl 5-pinacolboranylanthranilate: Methyl 5-pinacolboranylanthranilate was synthesized as described for methyl 4-pinacolboranylanthranilate using methyl 5-bromoanthranilate as the starting material.

Synthetic Procedure D: Synthesis of Methyl Heterorarylanthranilate

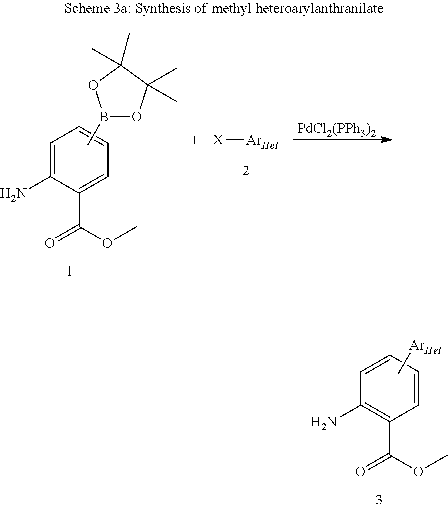

5. Methyl 4(or 5)-pinacolboranylanthranilate (Compound 1 in Scheme 3a, 0.416 g, 1.5 mmol) was mixed with heteroarylbromide or heteroaryliodide (Compound 2 in Scheme 3a with X═Br or I, 1 mmol) in a mixture of ethyl acetate (10 mL) and toluene (20 mL). PdCl₂(PPh₃)₂ (0.070 g, 0.1 mmol) was added followed by aqueous sodium carbonate (4 mL, 2N). The reaction mixture was purged with nitrogen and the reaction vessel sealed and heated at 120° C. for 100 minutes. The cooled mixture was poured into water and the organic layer was washed with brine, dried and evaporated to dryness to afford the title compound methyl 4(or 5)-heterorarylanthranilate (Compound 3 in Scheme 3a). The material was used in the following step without further purification.

Synthetic Procedure E: Synthesis of Heteroarylanthranilic Acid

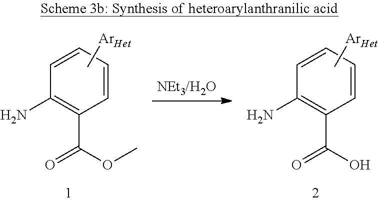

6. Mixture of methyl 4(or 5)-heteroarylantranilate (Compound 1 in Scheme 3b, 0.1 mol), triethylamine (5 mL), ethanol (50 mL) and water (50 mL) was refluxed for 36 hours. The reaction mixture was concentrated in vacuo on a rotary evaporator. To the residue water was added (10 mL) and the mixture was concentrated again. The latter procedure is repeated several times. So obtained crude 4(or 5)-heteroarylantranilic acid (Compounds 2 in Scheme 3b) was used in other synthesis without further purification.

Synthetic Procedure F: Synthesis of methyl (1H-imidazol-4-yl)anthranilate

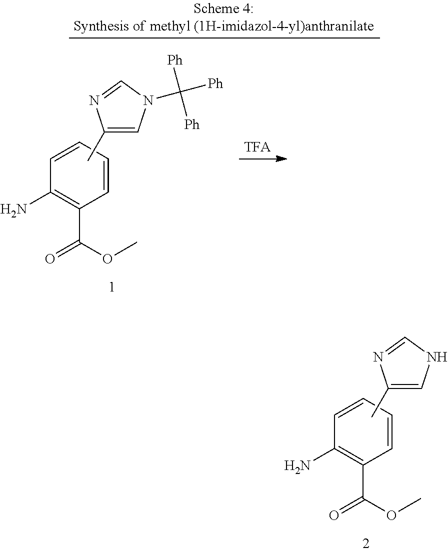

7. Synthesis of methyl 4-(1H-imidazol-4-yl)anthranilate: Methyl 4-(1-tritylimidazol-4-yl)anthranilate (Compound 1 in Scheme 4, 0.316 g, 0.69 mmol) was dissolved in a mixture of dichloromethane (12.8 mL) and trifluoroacetic acid (3.2 mL). The mixture was stirred at room temperature for 90 min. The solvent was removed under reduced pressure and the residue was partitioned between ethyl acetate and water. The organic layer was washed with bicarbonate followed by brine, then dried over magnesium sulfate and the solvent was removed. The residue was purified by flash chromatography to afford methyl 4-(1H-imidazol-4-yl)anthranilate (Compound 2 in Scheme 4, 0.134 g, ˜90% yield).

8. Synthesis of methyl 5-(1H-imidazol-4-yl)anthranilate: Methyl 5-(1H-imidazol-4-yl)anthranilate was synthesized as described for methyl 4-(1H-imidazol-4-yl)anthranilate using methyl 5-(1-tritylimidazol-4-yl)anthranilate as the starting material.

Synthetic Procedure G: Synthesis of 2-amino-4,5-disubstituted-thiophene-3-carbonitrile

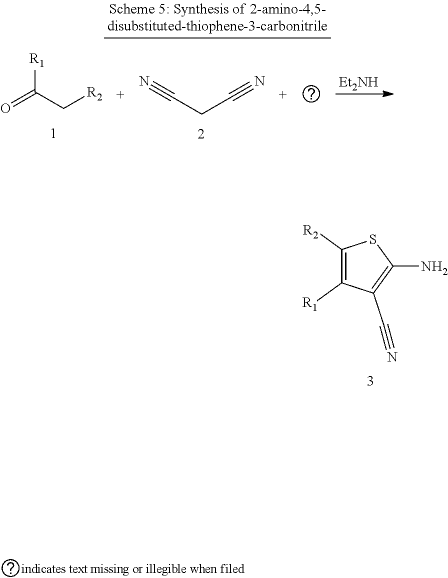

9. 2-Amino-4,5-disubstituted-thiophene-3-carbonitrile was prepared using the protocol from Eur. J. Med. Chem., 2010, 45(1), 69-77. To a stirring mixture of ketone (Compound 1 in Scheme 5, 0.1 mol), malononitrile (Compound 2 in Scheme 5, 0.1 mol) and powdered sulfur (0.1-0.11 mol) in ethanol (30 ml), diethylamine or morpholine (10 ml) was added dropwise, keeping the temperature lower than 50° C. After 1-3 hours the reactions were complete and the reaction mixtures were cooled in a fridge for crystallization. If no crystallization took place the mixtures were poured into 2-3 fold volume of water. The precipitates were filtered and recrystallized from ethanol to afford the target 2-amino-4,5-disubstituted-thiophene-3-carbonitrile (Compound 3 in Scheme 5).

Synthetic Procedure I: Synthesis of 4-ethynylphthalic acid

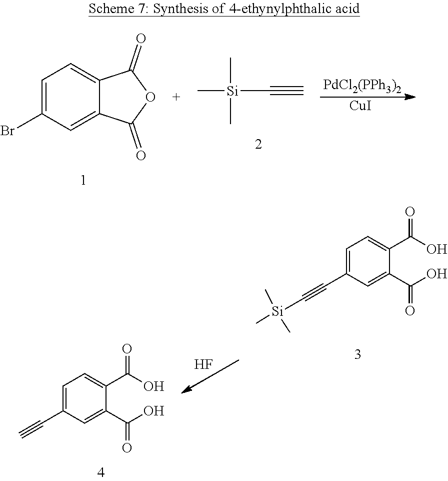

10. 4-Bromophthalic anhydride (Compound 1 in Scheme 7, 11.9 g, 52.4 mmol) and ethynyltrimethylsilane (Compound 2 in Scheme 7, 3.66 mL, 26.2 mmol) were mixed in THF (100 mL). PdCl₂(PPh₃)₂ (1.1 g, 1.6 mmol), triphenylphosphine (4.1 g, 15.7 mmol), copper(I) iodide (0.6 g, 3.1 mmol) and triethylamine (100 mL) were added to the mixture. The resulting reaction mixture was heated at 110° C. for 6 h. The solvents were removed under reduced pressure and the crude 4-[(trimethylsilyl)ethynyl]phthalic acid (Compound 3 in Scheme 7) was obtained.

11. Without additional purification the obtained crude material was dissolved in THF (200 mL) and was treated with 48% aqueous HF (7.6 mL) and triethylamine (37 mL). The mixture was stirred at room temperature for 1 h at which time LC/MS analysis indicated complete deprotection. The solvent was removed under reduced pressure. The residue was taken up in water and washed with a small quantity of dichloromethane to remove some organic-soluble impurities. The 4-ethynylphthalic acid (Compound 4 in Scheme 7) was recovered from the water by evaporation under reduced pressure. No further purification was performed.

Synthetic Procedure J: Synthesis of 4-(1H-1,2,3-triazol-4-yl)phthalic acid

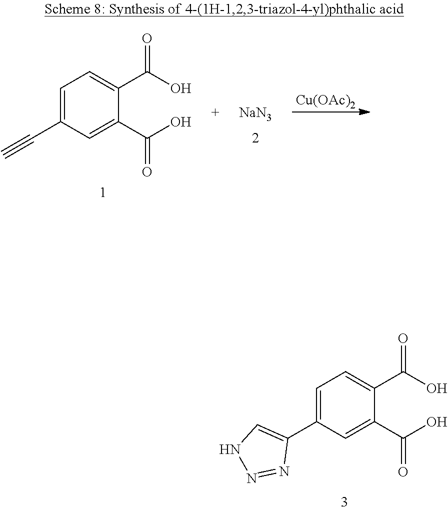 [SMILES: C#Cc1ccc(C(=O)O)c(C(=O)O)c1 ; "2" ; O=C(O)c1ccc(-c2c[nH]nn2)cc1C(=O)O ; "Cu(OAc)2" ; "NaN3"]

12. Crude 4-ethynylphthalic acid obtained according to synthetic procedure I (Compound 1 in Scheme 8, 9.0 g) was dissolved in DMF (150 mL) and water (60 mL). Sodium azide (3.62 g, 55.6 mmol), copper(II) acetate (0.94 g, 5.2 mmol) and sodium ascorbate (1.23 g, 6.23 mmol) were added to this solution. The mixture was stirred at 80° C. overnight. The solvents were removed under reduced pressure and the residue was purified by preparative HPLC to afford pure 4-(1H-1,2,3-triazol-5-yl)phthalic acid (Compound 3 in Scheme 8).

Synthetic Procedure J1: Synthesis of 4-(1-substituted-1H-1,2,3-triazol-4-yl)phthalic acid.

13. Using the protocol described in Procedure J, only replacing sodium azide (Compound 2 in Scheme 8) with different azide, 4-(1-substituted-1H-1,2,3-triazol-4-yl)phthalic acid were prepared from 4-ethynylphthalic acid (Compound 1 in Scheme 8). Several alkyl azides, aryl azides and heteroaryl azides were used to obtain corresponding 4-(1-alkyl-1H-1,2,3-triazol-4-yl)phthalic, 4-(1-aryl-1H-1,2,3-triazol-4-yl)phthalic and 4-(1-heteroaryl-1H-1,2,3-triazol-4-yl)phthalic acids.

Synthetic Procedure J2: Synthesis of 4-(5-substituted-1H-1,2,3-triazol-4-yl)phthalic acid.

14. First, crude 4-(substituted-ethynyl)anthranilic acid was obtained from 4-bromophthalic anhydride (Compound 1 in Scheme 7) using the first step of the Procedure I with ethynyltrimethylsilane (Compound 2 in Scheme 7) replaced with the corresponding monosubstituted acetylene. The obtained crude acid was used in the next step without additional purification.

Using the protocol described in Procedure J, only replacing 4-ethynylanthranilic acid (Compound 1 in Scheme 8) with obtained crude 4-(substituted-ethynyl)anthranilic acid, 4-(5-substituted-1H-1,2,3-triazol-4-yl)phthalic acid was prepared.

Synthetic Procedure J3: Synthesis of 5-(sulfonylaminocarbonyl)-1,3-dioxo-1,3-dihydro-2-benzofuran

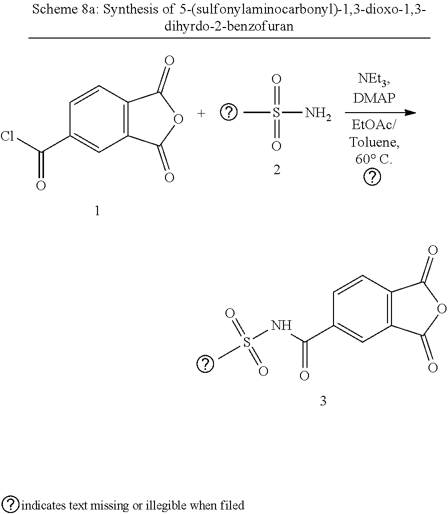

15. To a solution of alkylsulfonamide or arylsulfonamide (Compound 2 in Scheme 8a, 0.69 mmol) in ethyl acetate (1 mL) were added triethylamine (175.4 mg, 1.734 mmol), DMAP (4.28 mg, 0.035 mmol) and a solution of 1,3-dihydro-1,3-dioxoisobenzofuran-5-carbonylchloride (Compound 1 in Scheme 8a, 160 mg, 0.7627 mmol) in toluene (6 mL). The reaction mixture was stirred at 60° C. for 1.5 h. The solvent was evaporated and dried under vacuum to give crude 5-(sulfonylaminocarbonyl)-1,3-dioxo-1,3-dihydro-2-benzofuran (Compound 3 in Scheme 8a).

Synthetic Procedure K: Synthesis of N-biarylphthalimide-5-Carboxylic Acid

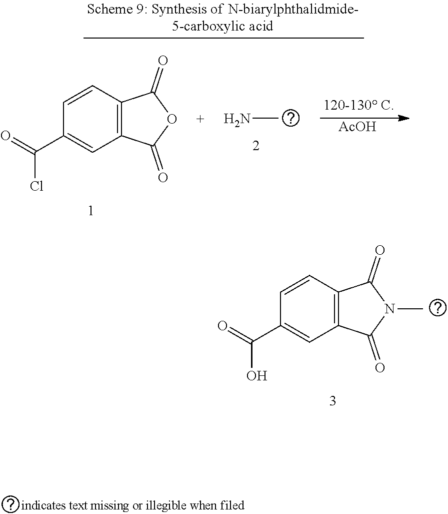

16. A mixture of trimellitic anhydride (Compound 1 in Scheme 9, 0.192 g, 1.0 mmol) and biarylamine (Compound 2 in Scheme 9, 1.1 mmol) in AcOH (1-5 mL) was heated to 120-130° C. for 2-4 h. The reaction was monitored by LC/MS. The solvent was concentrated by rotary evaporator. The crude product was dissolved in DMF (1 mL) and purified by preparative HPLC to give N-arylphthalimide-5-carboxylic acid (Compound 3 in Scheme 9).

17. Example: preparation of N-(4-hydroxy-[1,1′-biphenyl]-3-yl)phthalimide-5-carboxylic acid.

18. A mixture of trimellitic anhydride (1.0 mmol) and 3-amino-[1,1′-biphenyl]-4-ol (1.1 mmol) in AcOH (1-2 mL) was heated to 120-130° C. for 3 h. The reaction was monitored by LC/MS. The solvent was concentrated by rotary evaporator. The crude product was dissolved in DMF (1 mL) and purified by preparative HPLC to give N-(4-hydroxy-[1,1′-biphenyl]-3-yl)phthalimide-5-carboxylic acid (LC/MS=360.34 [MH+]).

Synthetic Procedure K1: Synthesis of Amides and Esters of N-Biarylphthalimide-5-Carboxylic Acid

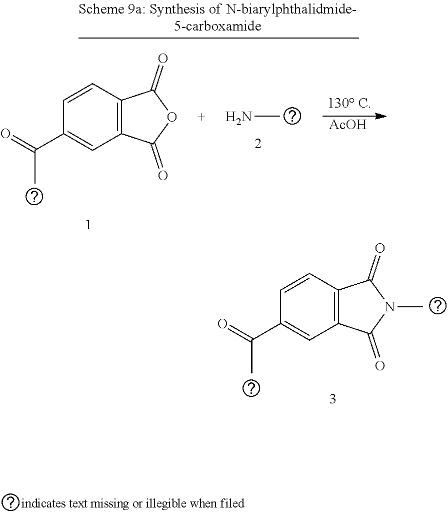

19. N-Biarylphthalimide-5-carboxamide (Compound 3 in Scheme 9a) was prepared using steps from synthetic procedure K, where 1,3-dioxo-1,3-dihydro-2-benzofuran-5-carboxamide (Compound 1 in Scheme 9a) was substituted for trimellitic anhydride (Compound 1 in Scheme 9). The reaction was carried at 130° C. in acetic acid for 2 h at which time LC/MS analysis showed the complete disappearance of the starting material. The solvent was removed under reduced pressure and the residue was taken up in a minimum of DMSO and purified by preparative HPLC to afford the pure N-biarylphthalimide-5-carboxamide (Compound 3 in Scheme 9a).

20. Same procedure, only using ester of 1,3-dioxo-1,3-dihydro-2-benzofuran-5-carboxylic acid as a starting material (Compound 1 in Scheme 9a), was used to obtain corresponding ester of N-biarylphthalimide-5-carboxylic acid.

Synthetic Procedure K2: Synthesis of 5-Sulfonylaminocarbonyl Derivatives of N-Biarylphthalimide

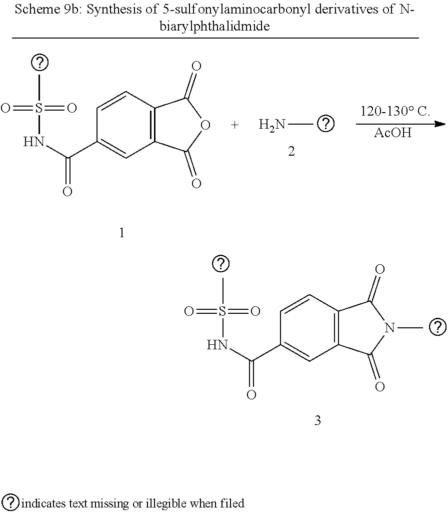

21. 5-Sulfonylaminocarbonyl derivatives of N-biarylphthalimide (Compound 3 in Scheme 9b) were prepared using steps from synthetic procedure K1. The reaction mixture of 5-(sulfonylaminocarbonyl)-1,3-dioxo-1,3-dihydro-2-benzofuran (Compound 1 in Scheme 9b, 0.2 mmol) and biarylamine (Compound 2 in Scheme 9b, 0.2 mmol) in acetic acid (3 ml) was stirred at 120-130° C. for 3 h. Acetic acid was evaporated, and the residue purified by prep-HPLC to give 5-sulfonylaminocarbonyl derivatives of N-biarylphthalide.

Synthetic Procedure L: Synthesis of N-biaryl-5-(1H-1,2,3-triazol-4-yl)phthalimide and variants thereof with substituted triazolyl

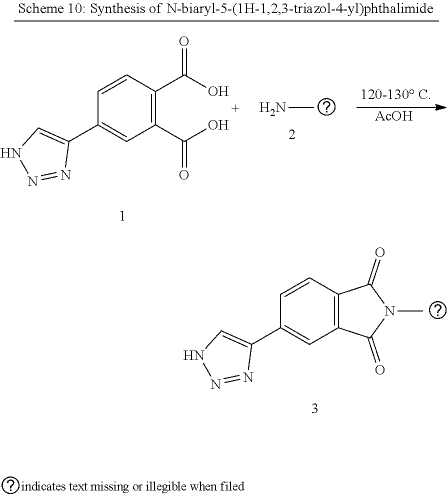

22. 4-(1H-[1,2,3]-Triazol-4-yl)phthalic acid (Compound 1 in Scheme 10, 0.233 g, 1.0 mmol) and biarylamine (Compound 2 in Scheme 10, 1.1 mmol) were dissolved in acetic acid and heated at reflux with stirring for two days. The solvent was removed under reduced pressure and the residue was purified by preparative HPLC to afford the corresponding N-biaryl-5-(1H-1,2,3-triazol-4-yl)phthalimide (Compound 3 in Scheme 10) in pure form.

23. Example: preparation of 3-(1,3-dioxo-5-(1H-1,2,3-triazol-4-yl)isoindolin-2-yl)-[1,1′-biphenyl]-4-carboxylic acid

24. 4-(1H-1,2,3-triazol-4-yl)phthalic acid (1.18 g, 5.06 mmol) and 3-amino-[1,1′-biphenyl]-4-carboxylic acid (1.09 g, 5.1 mmol) were dissolved in acetic acid (70 mL) and the mixture was heated with stirring for two days. The solvent was removed under reduced pressure and the residue was purified by preparative HPLC to afford 500 mg of target compound (LC/MS=411.1 [M+H]).

25. Following the same procedure, only using 4-{1 (or 5)-substituted-1H-[1,2,3]-triazol-4-yl}phthalic acid instead of 4-(1H-[1,2,3]-triazol-4-yl)phthalic acid, the corresponding N-biaryl-5-{1 (or 5)-substituted-1H-[1,2,3]-triazol-5-yl}phthalimide was obtained.

Synthetic Procedure M: Synthesis of N-biaryl-5-(1H-tetrazol-5-yl)phthalimide

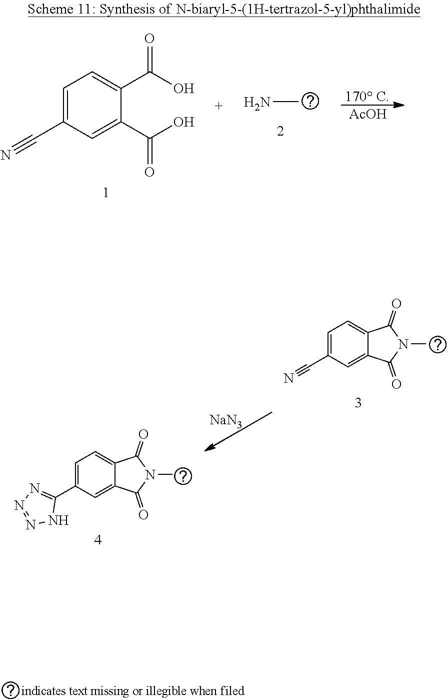

26. Step 1: 4-cyano-1,2-benzenedicarboxylic acid (Compound 1 in Scheme 11, 1.8 g, 9.37 mmol) and a biarylamine (Compound 2 in Scheme 11, 5.16 mmol) were dissolved in acetic acid (300 mL) and sealed in a pressure vessel. The mixture was heated at 170° C. for 30 minutes. Upon cooling, the solvent was removed and the residue was partitioned between ethyl acetate and water. The organic soluble material was washed with brine and the solvent removed to afford crude material which was purified by column chromatography to give pure N-biaryl-5-cyanophthalimide (Compound 3 in Scheme 11).

27. Step 2: N-biaryl-5-cyanophthalimide (Compound 3 in Scheme 11) was mixed with sodium azide (0.188 g, 2.89 mmol) and triethylamine hydrochloride (0.396 g, 2.89 mmol) in DMF (3.2 mL). The reaction mixture was stirred at 100° C. for 1 h at which time the reaction was done. The reaction mixture was partitioned between ethyl acetate and water. The organic layer was washed with dilute HCl followed by brine and the solvent was removed to afford crude N-biaryl-5-(1H-tetrazol-5-yl)phthalimide (Compound 4 in Scheme 11), which was purified by preparative HPLC.

Synthetic Procedure N: Synthesis of methyl 2-(6-bromoisoindolinon-2-yl)arylbenzoate

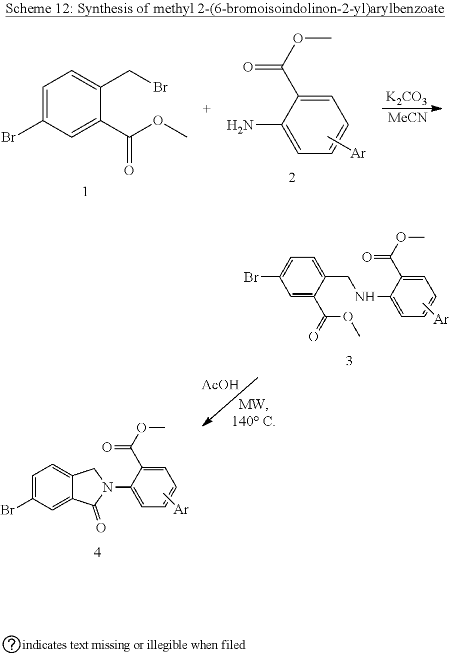

Synthesis of methyl 5-bromo-2-{[(2-(methoxycarbonyl)arylphenyl)amino]methyl}benzoate (Compound 3 in Scheme 12)

28. Methyl 5-bromo-2-(bromomethyl)benzoate (Compound 1 in Scheme 12, 2.05 g, 6.7 mmol) and methyl arylanthranilate (Compound 2 in Scheme 12, 6.7 mmol) were dissolved in acetonitrile (67 mL). Then potassium carbonate (1.85 g, 13.4 mmol) was added and the mixture was heated at 70° C. for 20 h. Upon cooling, the mixture was partitioned between ethyl acetate and water and the organic layer was separated. The aqueous layer was re-extracted with ethyl acetate and then the combined organic extracts were washed with brine and evaporated to dryness. The crude compound was purified by flash chromatography to afford the pure methyl 5-bromo-2-({[aryl-2-(methoxycarbonyl)phenyl]amino}methyl)benzoate.

Synthesis of methyl 2-(6-bromoisoindolinon-2-yl)arylbenzoate (Compound 4 in Scheme 12)

29. Methyl 5-bromo-2-({[aryl-2-(methoxycarbonyl)phenyl]amino}methyl)benzoate (Compound 3 in Scheme 17, 2.045 mmole) was dissolved in acetic acid (10 mL) and the mixture was heated at 140° C. in a microwave reactor for 4 h. Upon cooling, the reaction mixture was diluted in 1:1 ether-hexane mixture and the target compound precipitated. The solid was filtered and washed with ether-hexane to afford methyl 2-(6-bromoisoindolinon-2-yl)arylbenzoate.

Synthetic Procedure O: Synthesis of N-(4,5-disubstituted-3-cyanothiophen-2-yl)-6-bromoisoindolinone

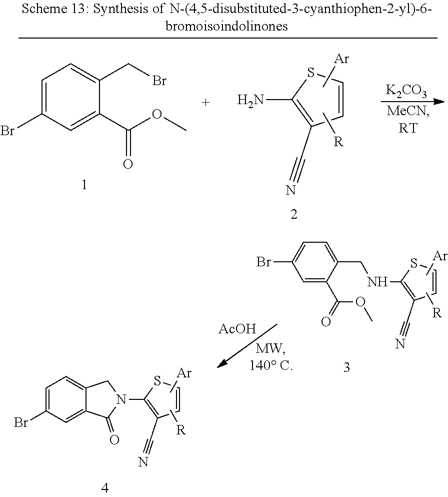

30. The target compound N-(4,5-disubstituted-3-cyanothiophen-2-yl)-6-bromoisoindolinone (Compound 4 in Scheme 13) was obtained using the same process described in synthetic procedure N with methyl arylanthranilate (Compound 3 in Scheme 12) replaced with 2-amino-4,5-disubstituted-thiophene-3-carbonitrile (Compound 2 in Scheme 13) and consequently the intermediate compound was methyl 5-bromo-2-{[(4,5-disubstituted-3-cyanothiophen-2-yl)amino]methyl)benzoate (Compound 3 in Scheme 13) instead of methyl 5-bromo-2-{[aryl-2-(methoxycarbonyl)phenylamino]methylabenzoate (Compound 4 in Scheme 12).

Synthetic Procedure P Synthesis of esters and amides of 2-(6-substituted-isoindolinon-2-yl)arylbenzoic and 2-(5-substituted-1,3-dioxo-1,3-dihydroisoindol-2-yl)arylbenzoic Acids

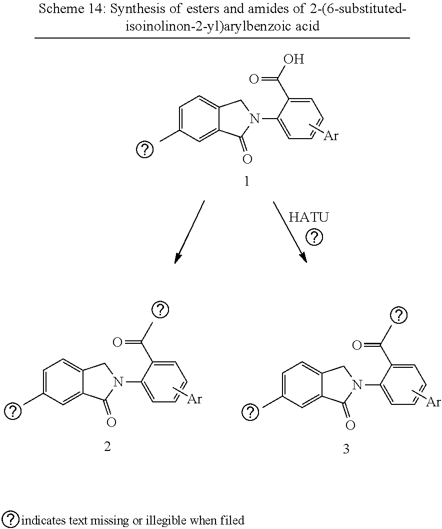

31. Various 2-(6-substituted-isoindolinon-2-yl)-5-arylbenzoic acids (shown as Compound 1 in Scheme 14) were transformed to corresponding esters and amides (Compound 2 and 3 in Scheme 14).

32. The method is exemplified for isoindolinone-derived compounds, however the same procedures were used to obtain esters and amides of 2-(5-substituted-1,3-dioxo-1,3-dihydroisoindol-2-yl)arylbenzoic acids.

Synthesis of Esters, Route A

33. The acid (Compound 1 in Scheme 14, 0.072 mmol), HATU (55 mg, 0.144 mmol) and diisopropylethylamine (27.9 mg, 0.216 mmol) were dissolved in DMF (1 mL) and the alcohol HORₑₛₜ (0.72 mmol), corresponding to the desired ester, was added. The reaction mixture was stirred overnight at room temperature. The mixture was poured into a mixture of ethyl acetate and dilute HCl. The organic layer was washed with water and brine solution, dried and evaporated to dryness to afford the crude product which was purified by preparative HPLC to afford the target ester (Compound 2 in Scheme 14).

For some esters alternative procedures were used as described below.

Synthesis of Alkyl Esters, Route B

34. To a solution of acid (Compound 1 in Scheme 14, 0.0463 mmol) in isopropyl alcohol (1 mL) was added concentrated H₂SO₄ (40 μL). The reaction mixture was stirred at 100° C. for 10 h. After reaction finished, water (0.1 mL) was added to reaction mixture. Then, isopropyl alcohol was evaporated. Water (10 mL) was added to mixture. The precipitate was filtered, and purified by prep-HPLC to get isopropyl ester.

The same procedure with isopropyl alcohol replaced with different C₂-C₈ alkyl alcohols, for example tert-butyl alcohol, was used to obtain corresponding alkyl esters.

Synthesis of (5-methyl-2-oxo-1,3-dioxol-4-yl)methyl esters, Route C

35. To a mixed solution of acid (Compound 1 in Scheme 14, 35 mg, 0.081 mmol) and EDAC-HCl (16 mg, 0.083 mmol) in DMF (0.6 ml) were added DMAP (15 mg, 0.121 mmol), and 4-(hydroxymethyl)-5-methyl-1,3-dioxol-2-one (73.8 mg, 0.567 mmol). The reaction mixture was stirred at room temperature for over weekend. After reaction finished, the reaction mixture was diluted with water (0.1 ml) and purified by prep-HPLC to get (5-methyl-2-oxo-1,3-dioxol-4-yl)methyl 2-(6-substituted-isoindolinon-2-yl)arylbenzoate.

Synthesis of Esters, Route D

36. To a solution of acid (Compound 1 in Scheme14, 0.081 mmol) in diethyl ether (1 mL) was added oxalyl chloride (10.5 mg, 0.083 mmol), mixture stirred at room temperature for overnight. Mixture evaporated to dryness under vacuum to afford crude 2-(6-substituted-isoindolinon-2-yl)-4-arylbenzoyl chloride which was used in the next step without purification.

37. 2-(6-Substituted-isoindolinon-2-yl)-4-arylbenzoyl chloride (prepared as described in the previous step) was added to a solution of triethylamine (16 mg, 0.162 mmol) in corresponding alcohol (1 mL). The mixture was stirred at RT for 2 h. The solvent was removed and the desired alkyl 2-(6-substituted-isoindolinon-2-yl)arylbenzoate (Compound 3 in Scheme 14) was purified by preparative HPLC.

Synthesis of Esters of Amino Alcohols, Route E

38. First, the N-Boc protected amino alcohol, corresponding to the desired ester was taken and N-Boc-aminoalkyl 2-(6-substituted-isoindolinon-2-yl)arylbenzoate was obtained as described in route A of this procedure. Then the obtained ester (0.072 mmol) was dissolved in TFA (4 mL) and held at room temperature for 30 min, after which time Boc-protection was cleaved. The solvent was removed and the desired aminoalkyl 2-(6-substituted-isoindolinon-2-yl)arylbenzoate (Compound 3, in Scheme 25) was purified by preparative HPLC.

Synthesis of (2-oxo-1,3-oxazolidin-5-yl)Methyl Ester, Route F

39. To a solution of acid (Compound 1 in Scheme14, 50 mg, 0.058 mmol) in dry dichloromethane (2 mL), 2-hydroxypyridine (11 mg, 0.11 mmol) and DCC (28.5 mg, 0.14 mmol) were added under N₂. After heating the reaction mixture at reflux overnight, 5-(hydroxymethyl)-1,3-oxazolidin-2-one (20 mg, 0.17 mmol) was added. After stirring for 5 h, the mixture was filtered and concentrated in vacuo. The residue was purified by preparative HPLC.

Synthesis of Amides

40. The acid (Compound 1 in Scheme 14, 0.072 mmol), HATU (55 mg, 0.144 mmol) and diisopropylethylamine (27.9 mg, 0.216 mmol) were dissolved in DMF (1 mL) and the amine (designated as HRₐₘ in Scheme 14, 0.72 mmol), corresponding to the desired amide, was added. The reaction mixture was stirred overnight at room temperature. The mixture was poured into a mixture of ethyl acetate and dilute HCl. The organic layer was washed with water and brine solution, dried and evaporated to dryness to afford the crude product which was purified by preparative HPLC to afford the target amide (Compound 3 in Scheme 14).

The method allows the use of amines in the form of hydrochlorides.

Synthetic Procedure Q: Synthesis of 2-(6-bromoisoindolinon-2-yl)arylbenzoic Acid

41. Methyl 2-(6-bromoisoindolinon-2-yl)arylbenzoate (0.13 mmol) was dissolved in a mixture of methanol THE (0.5 mL:0.5 mL) and treated with LiOH (0.33 mL aqueous, 2N, 0.66 mmol) at room temperature for 3 h, after which ethyl acetate (50 mL) and HCl (20 mL, 1 M) were added. The aqueous layer was re-extracted with more ethyl acetate and the combined organic layers were washed with brine, dried with Na₂SO₄, evaporated and used in the next step without additional purification.

Synthetic Procedure R: Synthesis of N-biarylisoindolinone-6-carboxylic Acid

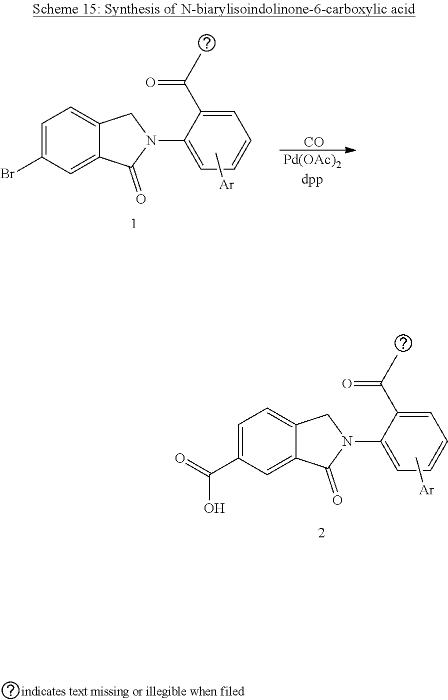

42. Different N-biarylisoindolinone-6-carboxylic acids (Compound 2 in Scheme 15) were synthesized from the corresponding N-biaryl-6-bromoisoindolinones (Compound 1 in Scheme 15). Rx together with the carbonyl it is attached to forms either carboxylic acid or carboxylic acid ester or carboxamide.

43. N-biaryl-6-bromoisoindolinones (Compound 1 in Scheme 15, 0.182 mmol), palladium acetate (37.6 mg, 0.167 mmol), dpp (75 mg, 0.182 mmol) and triethylamine (221 mg, 2.184 mmol) were mixed in DMSO (20 mL) and water (5 mL) in a sealed pressure vessel. The reaction mixture was heated at 100° C. under the atmosphere of carbon monoxide for 2 h. Upon cooling the mixture was partitioned between water and ethyl acetate and solid was filtered off. The aqueous layer was extracted again with ethyl acetate and the combined organic layers were dried over sodium sulfate and the solvent was removed. The residue was purified by HPLC to afford the target N-biarylisoindolinone-6-carboxylic acid (Compound 2 in Scheme 15).

Synthetic Procedure S: Synthesis of methyl 2-[6-(sulfonylaminocarbonyl)isoindolinon-2-yl]arylbenzoate and Corresponding Acid

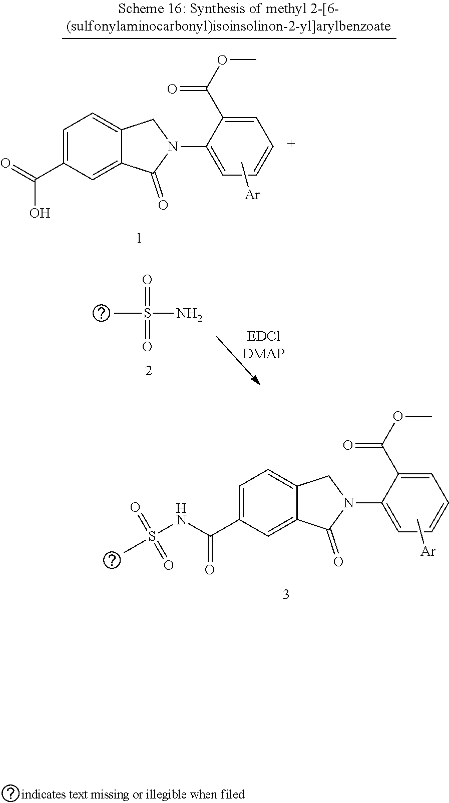

44. N-[2-(Methoxycarbonyl)arylphenyl]isoindolinone-6-carboxylic acid (Compound 1 in Scheme 16, 0.856 mmol), R_{S}-sulfonamide (Compound 2 in Scheme 16, 1.712 mmol), EDCI (328 mg, 1.712 mmol), and DMAP (314 mg, 2.57 mmol) were mixed in DMF (8 mL) and stirred overnight at room temperature. The mixture was acidified with HCl (1 N), poured into water and the solid was collected, washed with water followed by hexane, and then dried to afford the desired methyl 2-[6-(sulfonylaminocarbonyl)isoindolinon-2-yl]arylbenzoate (Compound 3 in Scheme 16).

45. Synthesis of 2-[6-(sulfonylaminocarbonyl)isoindolinon-2-yl]arylbenzoic acid

46. Methyl 2-[6-(sulfonylaminocarbonyl)isoindolinon-2-yl]arylbenzoate (0.665 mmol) was dissolved in methanol (3 mL) and THF (3 mL). Sodium hydroxide (3M aqueous, 0.67 mL, 2 mmol) was added to the mixture and the mixture was brought to reflux for 3 h. After cooling, HCl was added (20 mL, 1 N) and the desired acid precipitated. The solid was filtered, washed with water and hexane, and then dried to afford pure acid.

Synthetic Procedure S1: Synthesis of methyl 2-[6-(aminocarbonyl)isoindolinon-2-yl]arylbenzoate and Corresponding Acid

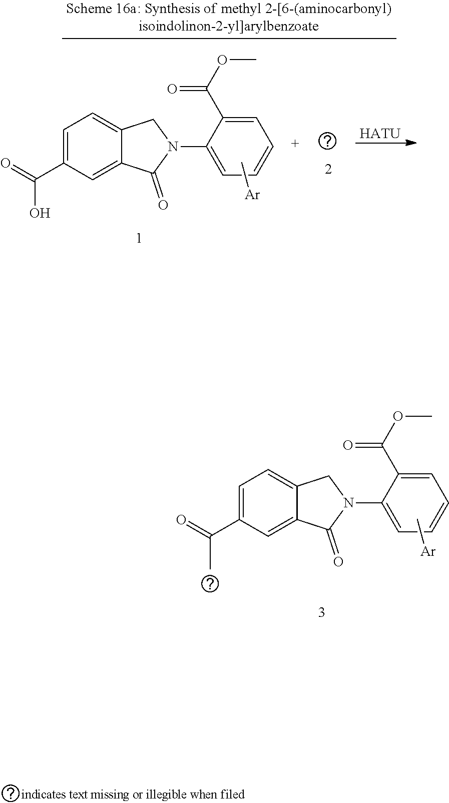

47. N-[2-(Methoxycarbonyl)arylphenyl]isoindolinone-6-carboxylic acid (Compound 1 in Scheme 16a, 0.072 mmol), HATU (55 mg, 0.144 mmol) and diisopropylethylamine (27.9 mg, 0.216 mmol) were dissolved in DMF (1 mL) and the amine (Compound 2 in Scheme 16a, 0.72 mmol), corresponding to the desired amide, was added. The reaction mixture was stirred overnight at room temperature. The mixture was poured into a mixture of ethyl acetate and dilute HCl. The organic layer was washed with water and brine solution, dried and evaporated to dryness to afford the crude product which was purified by preparative HPLC to afford the target amide (Compound 3 in Scheme 16a). The method allows the use of amines in the form of hydrochlorides.

48. Synthesis of 2-[6-(aminocarbonyl)isoindolinon-2-yl]arylbenzoic acid

49. Methyl 2-[6-(aminocarbonyl)isoindolinon-2-yl]arylbenzoate (Compound 3 in Scheme 16a, 0.665 mmol) was dissolved in methanol (3 mL) and THF (3 mL). Sodium hydroxide (3M aqueous, 0.67 mL, 2 mmol) was added to the mixture and the mixture was brought to reflux for 3 h. After cooling, HCl was added (20 mL, 1 N) and the desired acid precipitated. The solid was filtered, washed with water and hexane, and then dried to afford pure acid.

Synthetic Procedure T: Synthesis of methyl 2-(6-(1H-tetrazol-5-yl)isoindolinon-2-yl)arylbenzoate and Corresponding Acid

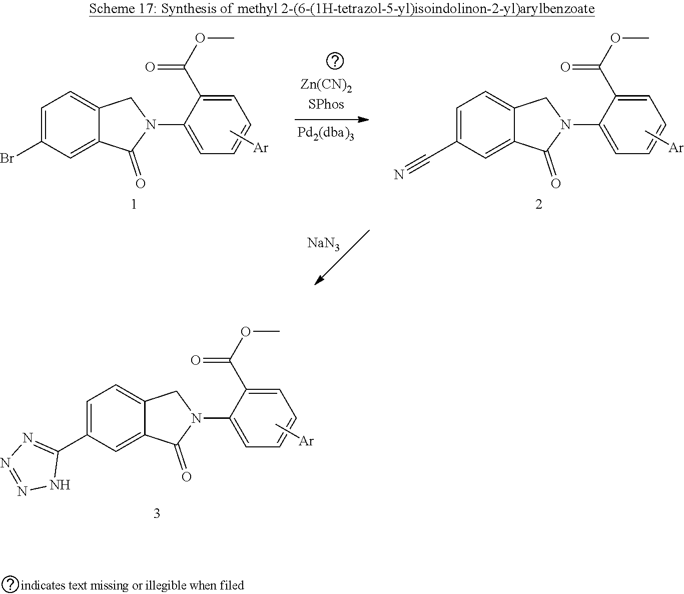

50. Methyl 2-(6-bromoisoindolinon-2-yl)arylbenzoate (Compound 1 in Scheme 17, 0.436 mmol), zinc cyanide (77 mg, 0.656 mmol), SPhos (36 mg, 0.089 mmol) and Pd₂(dba)₃ (40 mg, 0.0436 mmol) were mixed in DMF (7 mL) and water (50 μL) and heated in a microwave reactor at 120° C. for 1 h. Upon cooling, the reaction mixture was partitioned between ethyl acetate and water. The organic layer was washed with water and dried. Removal of the solvent gave the intermediate product methyl 2-(6-cyanoisoindolinon-2-yl)arylbenzoate (Compound 2 in Scheme 17) which was used without further purification.

51. Methyl 2-(6-cyanoisoindolinon-2-yl)arylbenzoate (0.3 mmol), sodium azide (58 mg, 0.9 mmol), and triethylamine hydrochloride (127.8 mg, 0.9 mmol) were dissolved in DMSO (3.5 mL) and heated in a microwave reactor at 140° C. for 90 min. Upon cooling, the reaction mixture was poured into water and HCl (2N, 5 mL) was added. The precipitated solid was filtered and dried under vacuum. Methyl 2-(6-(1H-tetrazol-5-yl)isoindolinon-2-yl)arylbenzoate (Compound 3 in Scheme 17) was used without further purification as a starting material in synthesis of corresponding acids, esters and amides. Pure methyl 2-(6-(1H-tetrazol-5-yl)isoindolinon-2-yl)arylbenzoate was also obtained using the HPLC purification.

52. Synthesis of 2-(6-(1H-tetrazol-5-yl)isoindolinon-2-yl)arylbenzoic acid

53. The crude methyl 2-(6-(1H-tetrazol-5-yl)isoindolinon-2-yl)arylbenzoate obtained as described above (Compound 3 in Scheme 17, 0.163 mmol) was dissolved in methanol (2.5 mL) and THF (1 mL). Then sodium hydroxide (2N, 0.41 mL) was added and the mixture stirred for 2 h at 45° C. at which time LC/MS analysis showed that the reaction was complete. The mixture was acidified to pH-2 with dilute HCl and the precipitated solid was collected and dried under high vacuum. HPLC purification was used to afford pure desired acid.

Synthetic Procedure U: Synthesis of methyl 2-(6-aminoisoindolinon-2-yl)arylbenzoate

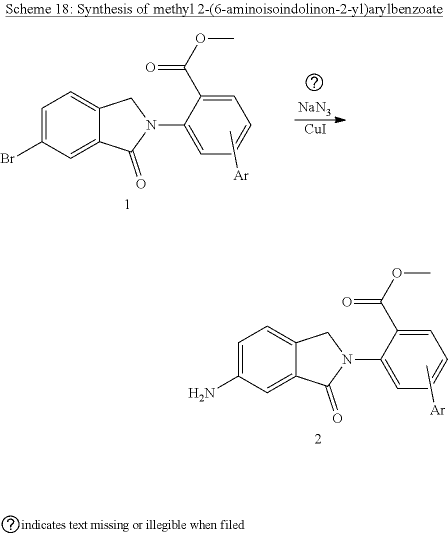

54. Methyl 2-(6-bromoisoindolinon-2-yl)arylbenzoate (Compound 1 in Scheme 18, 1.09 mmol) was mixed with CuI (260 mg, 1.365 mmol), sodium carbonate (231 mg, 2.18 mmol), sodium azide (178 mg, 2.725 mmol), and N¹,N²-dimethylethane-1,2-diamine (212 μL, 1.967 mmol) in DMSO (11 mL). The vial was degassed and heated in a microwave reactor at 110° C. for 1 h. Upon cooling, the reaction mixture was partitioned between ethyl acetate and water. The pH was adjusted to ˜4 with 2N HCl, the organic layer was washed with brine, dried and evaporated to dryness under high vacuum to afford the crude methyl 2-(6-aminoisoindolinon-2-yl)arylbenzoate (Compound 2 in Scheme 18) which was purified by flash chromatography.

Synthetic Procedure V: Synthesis of methyl 2-(6-carbonylaminoisoindolinon-2-yl)arylbenzoate and Corresponding Acid

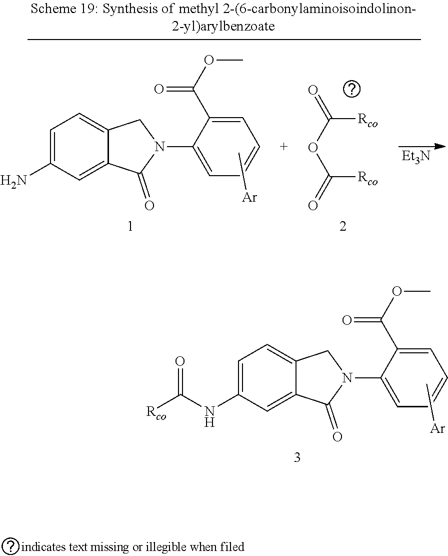

55. Methyl 2-(6-aminoisoindolinon-2-yl)arylbenzoate (Compound 1 in Scheme 19, 0.1015 mmol) was dissolved in THF (1 mL) and treated with suitable carboxylic acid anhydride (Compound 2 in Scheme 19, 0.505 mmol) and triethylamine (0.75 mmol). The solution was stirred overnight, after which the reaction mixture was partitioned between ethyl acetate and water. The organic solvent was removed under high vacuum to afford the crude methyl 2-(6-carbonylaminoisoindolinon-2-yl)arylbenzoate (Compound 3 in Scheme 19) which was used without further purification as a starting material in synthesis of corresponding acids, esters and amides as described below. Pure methyl 2-(6-carbonylaminoisoindolinon-2-yl)arylbenzoate was also obtained using the preparative HPLC.

56. Synthesis of 2-(6-carbonylaminoisoindolinon-2-yl)arylbenzoic acid

57. The crude methyl 2-(6-carbonylaminoisoindolinon-2-yl)arylbenzoate obtained as described above (Compound 3 in Scheme 19) was dissolved in methanol (1 mL) and THF (1 mL). Sodium hydroxide (2N, 250 μL) was added and the solution was heated at 40° C. for 90 min. 2N HCl was added to adjust the pH to ˜2 and the reaction mixture was partitioned between ethyl acetate and water. The organic layer was washed with water, dried and evaporated to dryness under high vacuum. The crude product was purified by preparative HPLC to afford 2-(6-carbonylaminoisoindolinon-2-yl)arylbenzoic acid.

Synthetic Procedure W: Synthesis of methyl 2-(6-sulfonylaminoisoindolinon-2-yl)arylbenzoate and Corresponding Acid

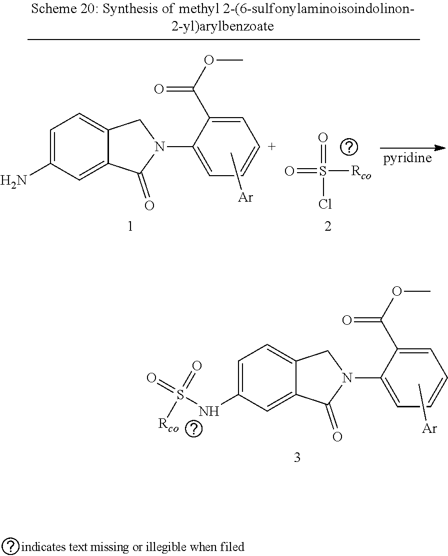

58. Methyl 2-(6-aminoisoindolinon-2-yl)arylbenzoate (Compound 1 in Scheme 20, 0.1015 mmol) was dissolved in THF (1 mL) and treated with suitable sulfonyl chloride (Compound 2 in Scheme 20, 39.2 μL, 0.505 mmol) and pyridine (69.5 μL, 68.2 mg, 0.75 mmol). The solution was stirred overnight, after which the reaction mixture was partitioned between ethyl acetate and water. The organic solvent was removed under high vacuum to afford the crude methyl 2-(6-sulfonylaminoisoindolinon-2-yl)arylbenzoate (Compound 3 in Scheme 20) which was used without further purification as a staring material in synthesis of corresponding acids, esters and amides as described below. Pure methyl 2-(6-sulfonylaminoisoindolinon-2-yl)arylbenzoate was also obtained using the preparative HPLC.

59. Synthesis of 2-(6-sulfonylaminoisoindolinon-2-yl)arylbenzoic Acid

60. The crude methyl 2-(6-sulfonylaminoisoindolinon-2-yl)arylbenzoate obtained as described above (Compound 3 in Scheme 20) was dissolved in methanol (1 mL) and THF (1 mL). Sodium hydroxide (2N, 250 μL) was added and the solution was heated at 40° C. for 90 min. 2N HCl was added to adjust the pH to ˜2 and the reaction mixture was partitioned between ethyl acetate and water. The organic layer was washed with water, dried and evaporated to dryness under high vacuum. The crude product was purified by preparative HPLC to afford 2-(6-sulfonylaminoisoindolinon-2-yl)arylbenzoic acid.

Synthetic Procedure X: Synthesis of methyl 2-(6-(1H-tetrazol-1-yl)isoindolinon-2-yl)arylbenzoate and Corresponding Acid and 5-Substituted-1H-tetrazol-1-yl Variants Thereof

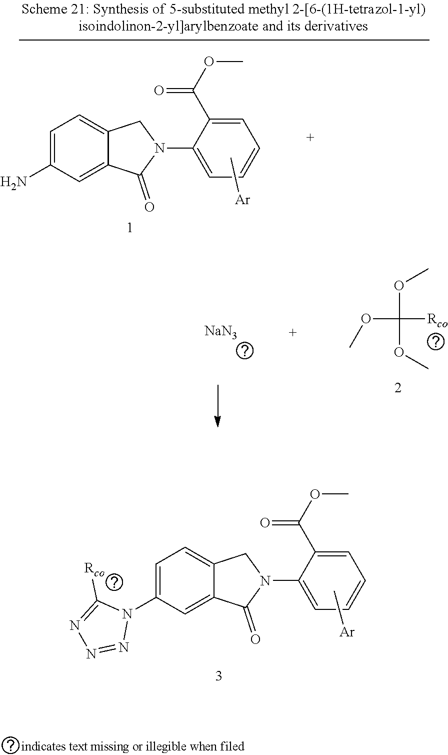

61. Methyl 2-(6-aminoisoindolinon-2-yl)arylbenzoate (Compound 1 in Scheme 21, 0.218 mmol), sodium azide (21.3 mg, 0.327 mmol) and suitable trimethyl orthoester (Compound 2 in Scheme 21, 0.329 mmol) were mixed in acetic acid (1 mL). The mixture was heated at 90° C. for 2 h, then cooled and poured into water. The precipitated solid was collected, washed with water, then washed with 3:7 ether-hexane mixture and dried. The obtained crude material was purified by preparative HPLC to afford methyl 2-(6-(5-substituted-1H-tetrazol-1-yl)isoindolinon-2-yl)arylbenzoate (Compound 3 in Scheme 21).

62. Methyl 2-(6-(1H-tetrazol-1-yl)isoindolinon-2-yl)arylbenzoate was synthesized as described above.

The only difference was replacing Rₙ₄ group in Scheme 21 with hydrogen and, respectively, using trimethyl orthoformate as Compound 2.

63. Synthesis of Tetrazole-Substituted Isoindolinonylarylbenzoic Acids

64. Methyl 2-(6-(5-substituted-1H-tetrazol-1-yl)isoindolinon-2-yl)arylbenzoate (Compound 3 in Scheme 21, 0.163 mmol) was dissolved in methanol (2.5 mL) and THF (1 mL). Sodium hydroxide (2N, 0.41 mL) was added and the mixture was heated at 45° C. for 2 h at which time LC/MS showed, that reaction was complete. The mixture was acidified to pH ˜2 with dilute HCl and the precipitated solid was collected, dried under high vacuum and purified by HPLC to afford pure 2-[6-(5-substituted-1H-tetrazol-1-yl)isoindolinon-2-yl]arylbenzoic acid.

65. Using the same procedure, 2-(6-(1H-tetrazol-1-yl)arylbenzoic acid was prepared from methyl 2-(6-(1H-tetrazol-1-yl)isoindolinon-2-yl)arylbenzoate.

Synthetic Procedure Y: Synthesis of Methyl 2-(6-ethynylisoindolinon-2-yl)arylbenzoate

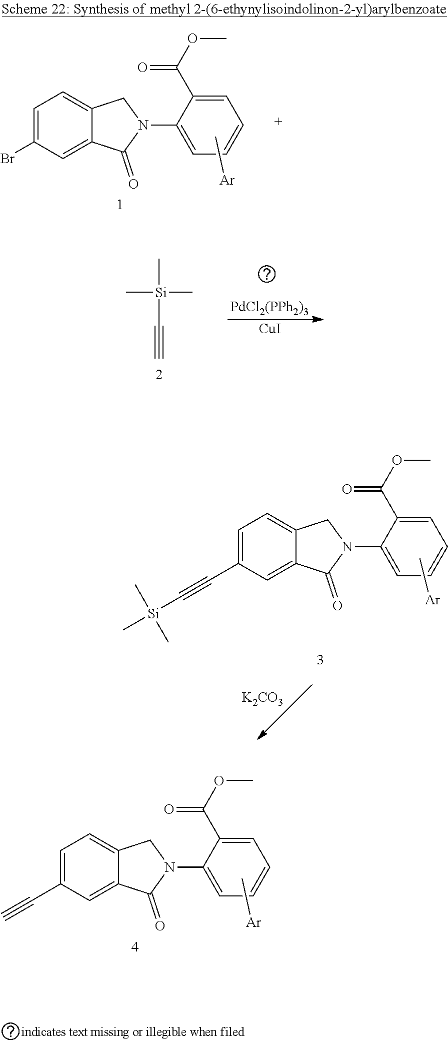

66. The synthesis of methyl 2-(6-ethynylisoindolinon-2-yl)arylbenzoate is performed in a two-step process as shown in Scheme 22.

67. Synthesis of methyl 2-(6-(trimethylsilylethynyl)isoindolinon-2-yl)arylbenzoate (Compound 3 in Scheme 22)

68. Methyl 2-(6-bromoisoindolinon-2-yl)arylbenzoate (Compound 1 in Scheme 22, 0.0507 mmol) was dissolved in DMF (0.5 mL). Ethynyltrimethylsilane (Compound 2 in Scheme 22, 24.8 mg, 0.2536 mmol), PdCl₂(PPh₃)₂ (3.5 mg, 0.0050 mmol), triethylamine (25.7 mg, 0.2536 mmol) and CuI (0.95 mg, 0.005 mmol) were added and the mixture was heated for 13 h at 100° C. Upon cooling, the mixture was acidified with dilute HCl and then partitioned between ethyl acetate and water. The organic layer was washed with bicarbonate solution, then dried and evaporated to dryness and the residue was purified by flash chromatography to afford pure target compound.

69. Synthesis of methyl 2-(6-ethynylisoindolinon-2-yl)arylbenzoate (Compound 4 in Scheme 22)

70. Methyl 2-(6-(trimethylsilylethynyl)isoindolinon-2-yl)arylbenzoate (Compound 3 in Scheme 22, 0.07044 mmol) was dissolved in methylene chloride (1 mL) and methanol (1 mL). Potassium carbonate (20 mg, 0.14088 mmol)) was added and the mixture was stirred at room temperature for 4 h at which time, methylene chloride was added and the solution was washed with dilute HCl followed by brine, then dried and evaporated to dryness to afford crude methyl 2-(6-ethynylisoindolinon-2-yl)arylbenzoate which was used without purification in other synthesis.

Synthetic Procedure Z: Synthesis of methyl 2-[6-(1H-1,2,3-triazol-5-yl)isoindolinon-2-yl]arylbenzoate and Corresponding Acid

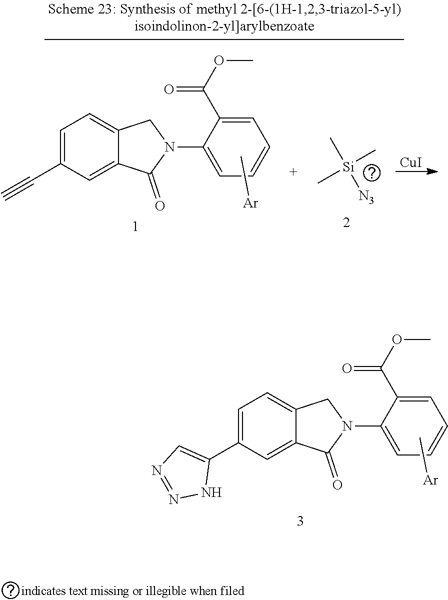

71. Crude methyl 2-(6-ethynylisoindolinon-2-yl)arylbenzoate obtained as described in synthetic procedure Y (Compound 1 in Scheme 23, 0.192 mmol) was mixed with trimethylsilyl azide (Compound 2 in Scheme 23, 111 mg, 0.964 mmol), and CuI (3.7 mg, 0.019 mmol) in DMF (2 mL) and methanol (0.2 mL). The mixture was heated in a microwave reactor at 100° C. for 5 h. Upon cooling the mixture was partitioned between water and ethyl acetate and solid was filtered off. The aqueous layer was extracted again with ethyl acetate and the combined organic layers were combined, dried over sodium sulfate and the solvent was removed. The residue was purified by HPLC to afford pure methyl 2-[6-(1H-1,2,3-triazol-5-yl)isoindolinon-2-yl]arylbenzoate (Compound 3 in Scheme 23)

72. Synthesis of 2-(6-(1H-1,2,3-triazol-5-yl)isoindolinon-2-yl)arylbenzoic acid

73. Methyl 2-[6-(1H-1,2,3-triazol-5-yl)isoindolinon-2-yl]arylbenzoate (0.665 mmol) was dissolved in methanol (3 mL) and THF (3 mL). Sodium hydroxide (3M aqueous, 0.67 mL, 2 mmol) was added and the mixture was brought to reflux for 3 h. After cooling, HCl was added (20 mL, 1 M) and the precipitated solid was filtered, washed with water and hexane and then dried to afford pure acid.

Synthetic Procedure AA: Coupling of aminoacids to 2-[3-cyano-4-(4-methoxyphenyl)-5-methylthiophen-2-yl]-1,3-dioxo-2,3-dihydro-1H-isoindole-5-Carboxylic Acid.

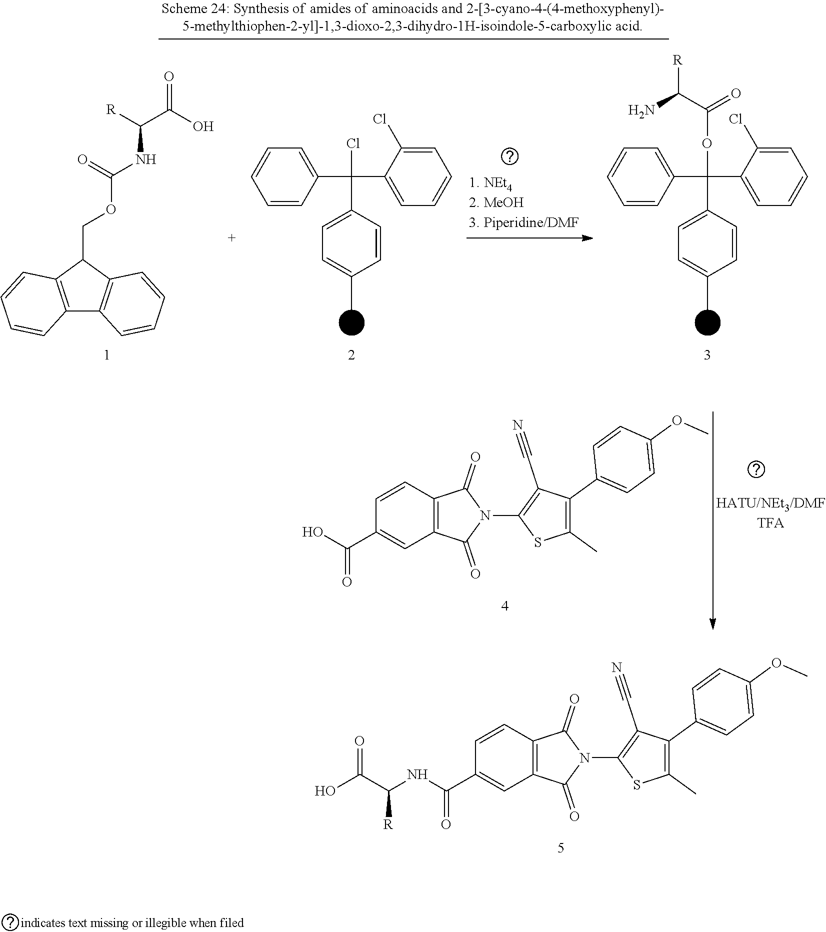

Preparation of 2-Chlorotrityl Polymer-Bound Aminoacids

74. Using a modification of the published procedure of Barlos et al., Tetrahedron Lett., 30, 3947 (1989), the N-Fmoc-protected aminoacid (Compound 1 in Scheme 24, 0.2 mmol) was dissolved in dichloromethane (2 mL) and treated with triethylamine (0.14 mL) followed by 2-chlorotrityl chloride resin (100-200 mesh) (Compound 2 in Scheme 24, 0.2 g). The reaction mixture was shaken at room temperature for 4 h. Methanol (1 mL) was added and the solution shaken at room temperature for 15 min to neutralize any unreacted resin. The resin was filtered and washed with DMF (2×5 mL). The Fmoc protecting group was removed by shaking the resin in a 20% solution of piperidine in DMF (3 mL) at room temperature 1 h. The resin was filtered and washed with DMF (3×10 mL) to afford the resin-bound aminoacid (Compound 3 in Scheme 24).

75. Coupling of polymer-bound aminoacids to 2-[3-cyano-4-(4-methoxyphenyl)-5-methylthiophen-2-yl]-1,3-dioxo-2,3-dihydro-1H-isoindole-5-carboxylic acid.

76. A solution of 2-[3-cyano-4-(4-methoxyphenyl)-5-methyl-thiophen-2-yl]-1,3-dioxo-2,3-dihydro-1H-isoindole-5-carboxylic acid (Compound 4 in Scheme 24, 0.542 g, 1.3 mmol) was dissolved in DMF (6 mL). To this solution was added HATU (0.608 g, 1.6 mmol) and triethylamine (0.365 g, 3.6 mmol). The solution (1 mL) was added to resin-bound aminoacids (obtained as described above, Compound 3 in Scheme 24) and shaken for 24 h at room temperature. The resin was filtered and washed with DMF (4 mL), methanol (4 mL), DMF (4 mL), and finally dichloromethane (4 mL). The product was cleaved from the resin by treatment with mixture TFA-dichloromethane (1:1, 4 mL) for 30 min at room temperature. The resin was filtered off and the residual solution was diluted with 5× volume of hexane and then evaporated to dryness under reduced pressure to afford the coupled product.

Synthetic Procedure AB: Synthesis of N-{2-[(aminoalkoxy)carbonyl]-4(or 5)-(1H-imidazol-4-yl)phenyl}phthalimide-5-Carboxylic Acid

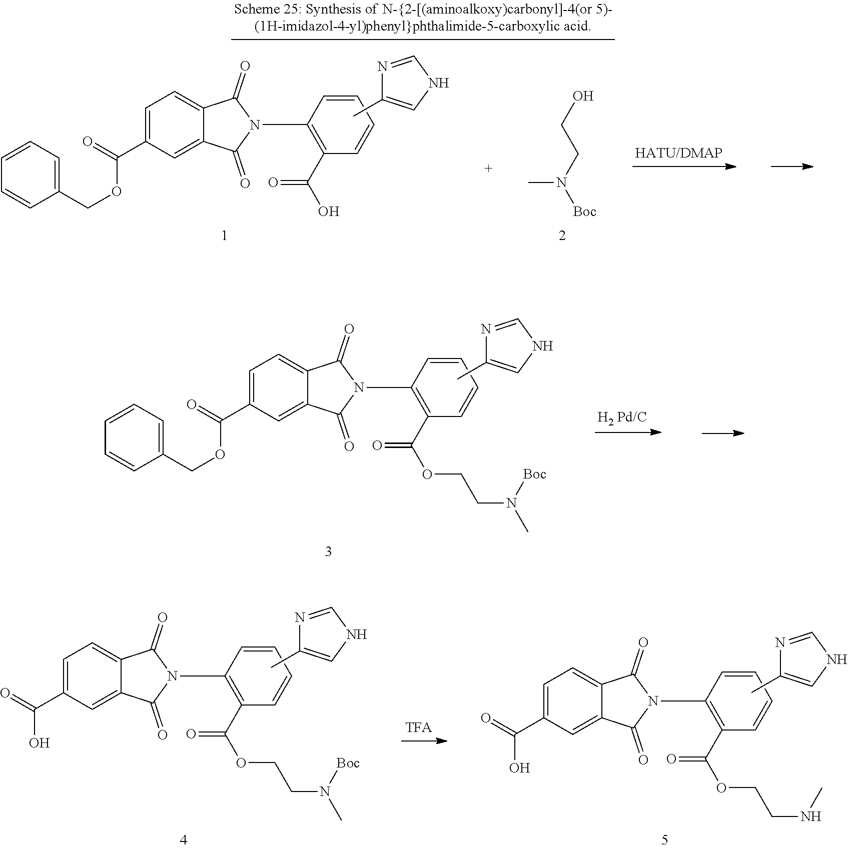

77. The aminoalcohol is exemplified in Scheme 25 using N-Boc-N-methyl-ethanolamine (Compound 2 in Scheme 25) and corresponding esters (Compounds 3 and 4 in Scheme 26), however over alcohols containing Boc-protected primary or secondary amines are suitable for this procedure.

Step 1: Synthesis of benzyl N-{2-[(N-Boc-aminoalkoxy)carbonyl]-4(or 5)-(1H-imidazol-4-yl)phenyl}phthalimide-5-carboxylate.

78. 2-{5-[(Benzyloxy)carbonyl]-1,3-dioxoisoindolin-2-yl}-4(or 5)-(1H-imidazol-4-yl)benzoic acid (Compound 1 in Scheme 25, 100 mg, 0.21 mmol) was dissolved in DMF (2.2 mL) and treated with HATU (122 mg, 0.32 mmol), triethylamine (89.5 μL, 0.64 mmol), 4-dimethylaminopyridine (2.6 mg, 0.021 mmol) and Boc-protected amino alcohol (Compound 2 in Scheme 25, 1.07 mmol), corresponding to the desired ester. The reaction mixture was stirred overnight at room temperature. The reaction mixture was poured into water and extracted with ethyl acetate, dried and evaporated to dryness. Purification by preparative HPLC afforded the pure product (Compound 3 in Scheme 25, ˜60% yield).

Step 2: Synthesis of N-{2-[(N-Boc-aminoalkoxy)carbonyl]-4(or 5)-(1H-imidazol-4-yl)phenyl}phthalimide-5-Carboxylic Acid

79. Benzyl N-{2-[(N-Boc-aminoalkoxy)carbonyl]-4(or 5)-(1H-imidazol-4-yl)phenyl}phthalimide-5-carboxylate (Compound 3 in Scheme 25, 0.35 mmol) was dissolved in methanol (9 mL) and hydrogenated at room temperature using 5% Pd/C for 7 h. LC/MS analysis showed the presence of the desired product mixed with some undesired methyl esters. Preparative HPLC was used to extract N-{2-[(N-Boc-aminoalkoxy)carbonyl]-4(or 5)-(1H-imidazol-4-yl)phenyl}phthalimide-5-carboxylic acid (Compound 4 in Scheme 25) from the mixture.

Step 3: Synthesis of N-{2-[(aminoalkoxy)carbonyl]-4(or 5)-(1H-imidazol-4-yl)phenyl}phthalimide-5-Carboxylic Acid.

80. N-{2-[(N-Boc-aminoalkoxy)carbonyl]-4(or 5)-(1H-imidazol-4-yl)phenyl}phthalimide-5-carboxylic acid (Compound 4 in Scheme 25, 0.19 mmol) was dissolved in TFA (4 mL) and held at room temperature for 30 min, after which time the solvent was removed and the product N-{2-[(aminoalkoxy)carbonyl]-4(or 5)-(1H-imidazol-4-yl)phenyl}phthalimide-5-carboxylic acid (Compound 5 in Scheme 25) was purified by preparative HPLC.

Synthetic Procedure AC: synthesis of 2-[1,3-dioxo-5-(1H-1,2,3-triazol-5-yl)isoindolin-2-yl]arylpyridine 1-oxide

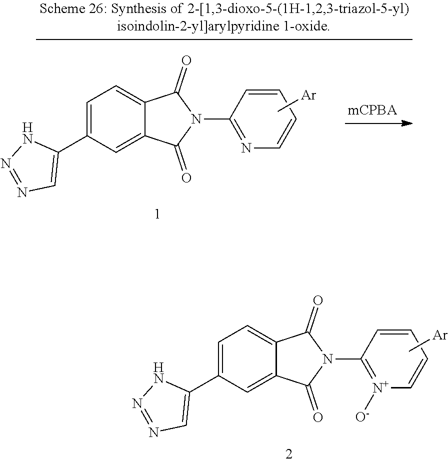

81. To a solution of 2-(arylpyridin-2-yl)-5-(1H-1,2,3-triazol-5-yl)isoindoline-1,3-dione (Compound 1 in Scheme 26, 0.26 mmol) in chloroform (10 mL) kept at 15° C. solid mCPBA (79% purity, 194.8 mg) was added. The reaction mixture was heated to 65° C. for 1 h. At this point, another 100 mg of mCPBA was added and the mixture heated for additional 40 min. The cooled reaction mixture was diluted with saturated sodium sulfite solution (30 mL) and water (30 mL). The mixture was extracted twice with dichloromethane. The combined organic mixture was washed with bicarbonate solution, water and then dried and evaporated. The residue was then purified by preparative HPLC to afford pure 2-[1,3-dioxo-5-(1H-1,2,3-triazol-5-yl)isoindolin-2-yl]arylpyridine 1-oxide (Compound 2 in Scheme 26).

Synthetic Procedure AD: synthesis of esters of N-biarylphthalimide-5-carboxylic and 2-biarylisoindolinone-6-Carboxylic Acids

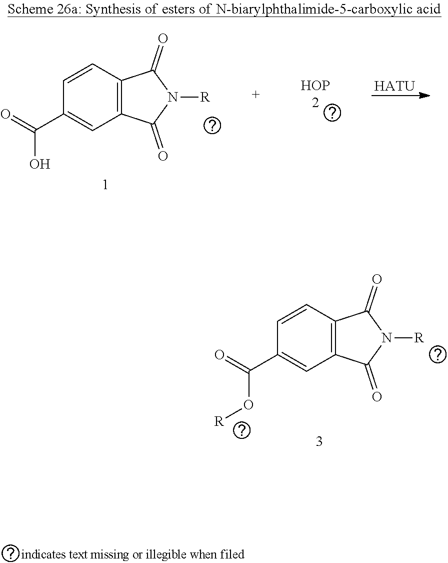

82. The acid (Compound 1 in Scheme 26a, 0.072 mmol), HATU (55 mg, 0.144 mmol) and diisopropylethylamine (27.9 mg, 0.216 mmol) were dissolved in DMF (1 mL) and the alcohol (Compound 2 in Scheme 26a, 0.72 mmol), corresponding to the desired ester, was added. The reaction mixture was stirred overnight at room temperature. The mixture was poured into a mixture of ethyl acetate and dilute HCl. The organic layer was washed with water and brine solution, dried and evaporated to dryness to afford the crude product which was purified by preparative HPLC to afford the target ester (Compound 3 in Scheme 26a).

Same procedure, only using 2-biarylisoindolinone-6-carboxylic acid instead of N-biarylphthalimide-5-carboxylic acid as a starting material, was used to obtain esters of 2-biarylisoindolinone-6-carboxylic acid.

The N-biarylphthalimide-5-carboxylic and 2-biarylisoindolinone-6-carboxylic acids used in this procedure did not contain additional carboxylic groups within biaryl substitutent.

Synthetic Procedure AE: Synthesis of 2-[6-(alcoxycarbonyl)isoindolinon-2-yl]-arylbenzoic acid

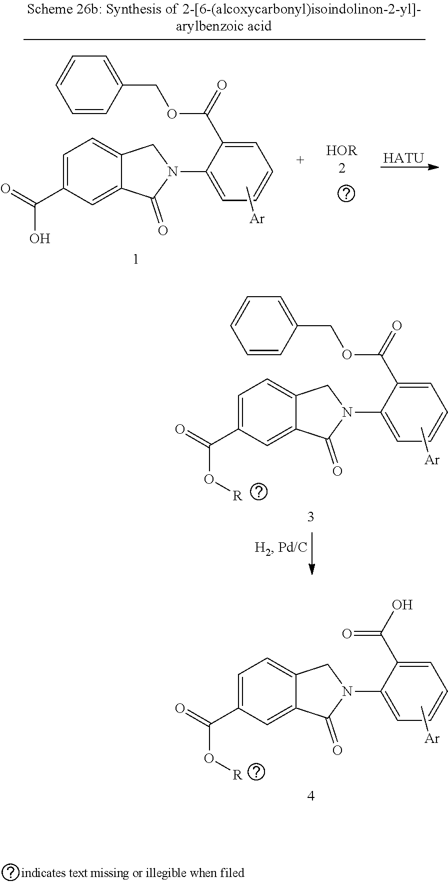

Step 1: Synthesis of alkyl ester of 2-{2-[(benzyloxy)carbonyl]arylphenyl}isoindolinone-6-Carboxylic Acid

83. Alkyl ester of 2-{2-[(benzyloxy)carbonyl]arylphenyl}isoindolinone-6-carboxylic acid (Compound 3 in Scheme 26b) was prepared as described in synthetic procedure AD from 2-{2-[(benzyloxy)carbonyl]arylphenyl}isoindolinone-6-carboxylic acid (Compound 1 in Scheme 26b) and corresponding alcohol (Compound 2 in Scheme 26b)

Step 2: Synthesis of 2-[6-(alcoxycarbonyl)isoindolinon-2-yl]-arylbenzoic acid

84. Alkyl ester of 2-{2-[(benzyloxy)carbonyl]arylphenyl}isoindolinone-6-carboxylic acid (Compound 3 in Scheme 26b, 0.56 mmol) was dissolved in methanol (10 mL) and hydrogenated at room temperature using 5% Pd/C for 10 h. The catalyst was removed by filtration and the solvent was evaporated. The residue was purified by preparative HPLC to afford pure 2-[6-(alcoxycarbonyl)isoindolinon-2-yl]-arylbenzoic acid (Compound 4 in Scheme 26b).

Analytical LC/MS

85. Analytical LC/MS was performed using two methods.

86. Method A: Waters Cortex C18 2.7 μM column (3.0×50 mm) was used at a flow rate of 1.2 mL/min, mobile phase: (A) water with 0.1% TFA, mobile phase, (B) acetonitrile with 0.1% TFA; retention times are given in minutes. Gradient: 5% B to 100% B over 4 min, with a stay at 100% B for 0.5 min, then equilibration to 5% B over 1.5 min.

87. Method B: Waters BEH C18 1.7 μM column (2.1×50 mm) was used at a flow rate of 0.3 mL/min, mobile phase: (A) water with 0.1% formic acid, mobile phase, (B) acetonitrile with 0.1% formic acid; retention times are given in minutes. Gradient: Stay at from 30 to 50% B for 0.5 min, then B to 100% B over 1.5 min, with a stay at 100% B for 0.5 min, then equilibration to initial level of B over 0.1 min.

Preparative HPLC

88. Preparative HPLC was performed using Higgins CLIPEUS C18 10 μm (30×100 mm) column at room temperature. The columns were used at a flow rate of 40 mL/min. The mobile phase was drawn from two solvent reservoirs containing (A) water with 0.1% TEA and (B) acetonitrile with 0.1% TEA. Gradient: 10% B to 70% B over 16 m, ramp up to 100% B and hold for 2 minutes, then equilibration to 10% B and hold for 2 minutes.

89. Commercially available starting materials Synthesis was performed using the following commercially available starting materials (in the table below the names of commercial providers are indicated for the reference only as one of the possible sources, the actual materials could have been obtained from other source):

| | CAS or MLD | Catalog number, |
|---|---|---|
| Compound | number | provider |
| 1-(2-hydroxyethyl)pyrrolidine | 2955-88-6 | H29404, Aldrich |
| 1-(4-methoxyphenyl)propan-1-one | 121-97-1 | QA-2980, Combi-Blocks, Inc. |
| 1-(dimethylamino)propan-2-ol | 108-16-7 | 471526, Aldrich |
| 1,2-diphenylethan-1-one | 451-40-1 | D4369, Aldrich |
| 1,3-dioxo-1,3-dihydro-2-benzofuran-5-carbonyl | 1204-28-0 | OR63040, Apollo Scientific |
| chloride | | |
| 1,3-dioxo-1,3-dihydro-2-benzofuran-5-carboxamide | MFCD11939097 | 143267, Matrix Scientific |
| 1-bromo-2,4,5-trimethylbenzene | 5469-19-2 | 211419, Aldrich |
| 2-amino-4-bromobenzonitrile | 304858-65-9 | 066621, Matrix Scientific |
| 2-amino-4-methoxybenzonitrile | 38487-85-3 | 079678, Fluorochem Ltd |
| 2-amino-4-phenylpyridine | 60781-83-1 | AK-30051, Ark Pharm, Inc. |
| 2-amino-4-(thiophen-2-yl)benzoic acid | 948006-04-0 | H34049, Alfa Aesar |
| 2-amino-5-bromobenzonitrile | 39263-32-6 | 642827, Aldrich |
| 2-amino-5-phenylpyridine | 33421-40-8 | CDS002679, Aldrich |
| 2-chlorotrityl chloride resin (100-200 mesh) | 42074-68-0 | 03498, Chem-Impex International |
| 2-dimethylaminoethanol | 108-01-0 | 471453, Aldrich |
| 2-hydroxypyridine | 142-08-5 | H56800, Aldrich |
| 2-methoxy[1,1′-biphenyl]-4-amine | 56970-24-2 | sc-335239, Santa Cruz |
| | | Biotechnology, Inc. |
| 2-methoxyethanol | 109-86-4 | 284467, Sigma-Aldrich |
| 6-methyl-4-phenylpyridin-2-amine | 73776-28-0 | AK364361, Ark Pharm, Inc. |
| 2,3,4-trifluorophenylboronic acid | 226396-32-3 | 524085, Aldrich |
| 2,4-dichlorophenylboronic acid | 68716-47-2 | 521388, Aldrich |
| 2,4-difluorophenylboronic acid | 144025-03-6 | 465070, Aldrich |
| 2,4,5-trifluorophenylboronic acid | 247564-72-3 | 524689, Aldrich |
| 3-amino-2-hydroxy-5-phenylpyridine | — | A02.248.559, Aurora Fine |
| | | Chemicals LLC |
| 3-amino-2-hydroxy-6-phenylpyridine | 203578-26-1 | DVL-0049, Suzhou Devi Pharma |
| | | Technology Co. Ltd. |
| 3-amino-4′-methoxy[1,1′-biphenyl]-4-carboxylic acid | 861389-74-4 | L-3249, AURUM Pharmatech LLC |
| 3-amino[1,1′-biphenyl]-4-carboxylic acid | 4445-43-6 | 066584, Fluorochem Ltd |
| 3-amino[1,1′-biphenyl]-4-ol | 1134-36-7 | AK135459, Ark Pharm, Inc., IL |
| 3-dimethylamino-1-propanol | 3179-63-3 | D144401, Aldrich |
| 4-chloro-3-fluorophenylboronic acid | 137504-86-0 | C2749, TCI America |
| 3-fluoro-4-methoxyphenylboronic acid | 149507-26-6 | 564036, Aldrich |
| 3-fluorophenylboronic acid | 768-35-4 | 441643, Aldrich |
| 3,4-difluorophenylboronic acid | 168267-41-2 | 465089, Aldrich |
| 3,4-dimethylaniline | 95-64-7 | 126373, Aldrich |
| 4-(2-hydroxyethyl)morpholine | 622-40-2 | H28203, Aldrich |
| 4-(4-fluorophenyl)pyridin-2-amine | 1159815-36-7 | AK216293, Ark Pharm, Inc. |
| 4-(hydroxymethyl)-5-methyl-1,3-dioxol-2-one | 91526-18-0 | 228148, Fluorochem Ltd |
| 4-bromophthalic anhydride | 86-90-8 | B1693, TCI America |
| 4-chloro-3-methylpyridine hydrochloride | 19524-08-4 | 632872, Aldrich |
| 4-cyano-1,2-benzenedicarboxylic acid | 830320-86-0 | AGN-PC-02OP8F, Angene |
| | | International Limited |
| 4-fluorophenylboronic acid | 1765-93-1 | 417556, Aldrich |
| 4-iodo-1-(triphenylmethyl)-1H-imidazole | 96797-15-8 | L510548, Aldrich |
| 4-methylphenylboronic acid | 5720-05-8 | 393622, Aldrich |
| 4-toluenesulfonyl chloride | 98-59-9 | 89730, Sigma-Aldrich |
| 5-(hydroxymethyl)-1,3-oxazolidin-2-one | 7517-99-9 | ST-7020, Combi-Blocks, Inc. |
| 5-iodo-1-methyl-1H-imidazole | 71759-88-1 | 679739, Aldrich |
| 6-aminopyridine-3-boronic acid pinacol ester | 827614-64-2 | 640379, Aldrich |
| acetic anhydride | 108-24-7 | 320102, Sigma-Aldrich |
| azidomethyl pivalate | 872700-68-0 | 758000, Aldrich |
| benzenesulfonamide | 98-10-2 | 108146, Aldrich |
| benzyl azide solution | 622-79-7 | 742430, Aldrich |
| bis(pinacolato)diboron | 73183-34-3 | 473294, Aldrich |
| Boc-L-valine methyl ester | 58561-04-9 | 466468, Aldrich |
| butan-2-ol | 78-92-2 | 294810, Sigma-Aldrich |
| butane-1-sulfonamide | 3144-04-5 | AK104989, Ark Pharm, Inc. |
| ethynyltrimethylsilane | 1066-54-2 | 218170, Aldrich |
| Fmoc-L-leucine | 35661-60-0 | 408611, Aldrich |
| Fmoc-L-phenylalanine | 35661-40-6 | 338338, Aldrich |
| Fmoc-L-valine | 68858-20-8 | 47638, Aldrich |
| glycerol | 56-81-5 | G5516, Sigma |
| isopropanol | 67-63-0 | W292907, Aldrich |
| malononitrile | 109-77-3 | M1407 Aldrich |
| methanesulfonamide | 3144-09-0 | 471534, Aldrich |
| methanesulfonyl chloride | 124-63-0 | 471259, Aldrich |
| methyl 2-amino-4-bromobenzoate | 135484-83-2 | 083232, Matrix Scientific |
| methyl 2-amino-5-bromo-4-methoxybenzoate | 169044-96-6 | AK206490, Ark Pharm, Inc. |
| methyl 2-amino-5-bromobenzoate | 52727-57-8 | 528811, Aldrich |
| methyl 3-amino[1,1′-biphenyl]-4-carboxylate | 800375-15-9 | AK314276, Ark Pharm, Inc. |
| methyl 5-bromo-2-methylbenzoate | 79669-50-4 | ACM79669504, Alfa Chemistry |
| N-Boc-ethanolamine | 26690-80-2 | 382027 Aldrich |
| N-Boc-L-serine | 3262-72-4 | 15500, Aldrich |
| N-Boc-N-methyl-ethanolamine | 57561-39-4 | H66660, Alfa Aesar |
| n-butanol | 71-36-3 | 281549, Sigma-Aldrich |
| N-Fmoc-L-serine | 73724-45-5 | 47601, Aldrich |
| N-Fmoc-L-tyrosine | 92954-90-0 | 47751, Aldrich |
| N,N′-Dicyclohexylcarbodiimide | 538-75-0 | D80002, Aldrich |
| N-alpha-Fmoc-L-asparagine | 71989-16-7 | 041290, Matrix Scientific |
| Fmoc-L-glutamine | 71989-20-3 | 47626, Aldrich |
| Fmoc-L-tryptophan | 35737-15-6 | 47637, Aldrich |
| phenylboronic acid | 98-80-6 | P20009, Aldrich |
| phenylmethanol | 100-51-6 | 402834, Sigma-Aldrich |
| pyrazine-2-boronic acid | 762263-64-9 | AK109132, Ark Pharm, Inc. |
| 1H-pyrazole-4-boronic acid | 763120-58-7 | 101553, Matrix Scientific |
| pyridine-3-boronic acid | 1692-25-7 | 512125, Aldrich |
| pyrimidine-5-boronic acid | MFCD03002366 | CDS013999, Aldrich |
| tert-butanol | 75-65-0 | 360538, Sigma-Aldrich |
| trimellitic anhydride | 552-30-7 | C0046, TCI America |

### Intermediate 1a: Methyl 5-bromo-2-(bromomethyl)benzoate

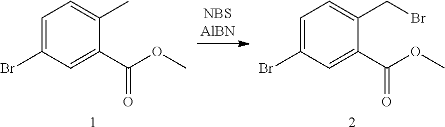

90. To a solution of methyl 5-bromo-2-methylbenzoate (1.5 g, 6.548 mmol) in CCl₄ (35 mL) was added NBS (1.4 g, 7.86 mmol) followed by AIBN (65 mg, 0.393 mmol). The reaction mixture was heated at reflux for 6 h. After reaction finished, the reaction mixture was poured into water and extracted with dichloromethane (50 mL×3). The combined organic layers were dried with Na₂SO₄, filtered, concentrated. The residue was purified by chromatography (silica gel, hexane/EtOAc=100/0-20/80) to give methyl 5-bromo-2-(bromomethyl)benzoate (1.513 g, 75%).

### Intermediate 1b: Methyl 3-amino-3′,4′-difluoro[1,1′-biphenyl]-4-carboxylate

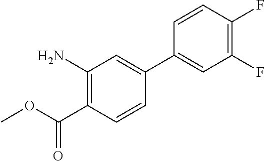 [SMILES: COC(=O)c1ccc(-c2ccc(F)c(F)c2)cc1N]

91. Methyl 3-amino-3′,4′-difluoro[1,1′-biphenyl]-4-carboxylate was prepared as described in synthetic procedure A from methyl 2-amino-4-bromobenzoate and 3,4-difluorophenylboronic acid.

### Intermediate 1c: 3-({[4-bromo-2-(methoxycarbonyl)phenyl]methyl}amino)-3′,4′-difluorobiphenyl-4-carboxylic acid methyl ester

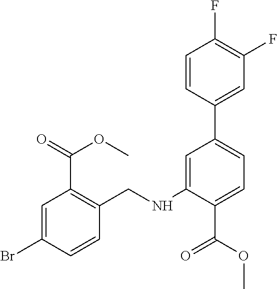 [SMILES: COC(=O)c1cc(Br)ccc1CNc1cc(-c2ccc(F)c(F)c2)ccc1C(=O)OC]

92. The title compound was prepared as described in first step of synthetic procedure N from methyl 5-bromo-2-(bromomethyl)benzoate (Intermediate 1a) and methyl 3-amino-3′,4′-difluoro[1,1′-biphenyl]-4-carboxylate (Intermediate 1b). MS m/z: (M+H)⁺ calculated for C₂₂H₁₄BrF₂NO₃: 459.26. found 459.14. LC/MS retention time: 2.23 minutes.

### Intermediate 1d: 3-Amino-3′,4′-difluoro[1,1′-biphenyl]-4-carboxylic Acid

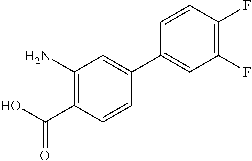 [SMILES: Nc1cc(-c2ccc(F)c(F)c2)ccc1C(=O)O]

93. 3-Amino-3′,4′-difluoro[1,1′-biphenyl]-4-carboxylic acid was prepared as described in synthetic procedure B from methyl 3-amino-3′,4′-difluoro[1,1′-biphenyl]-4-carboxylate (Intermediate 1b).

### Intermediate 2: 3-(6-Bromo-1-oxo-1,3-dihydroisoindol-2-yl)-3′,4′-difluorobiphenyl-4-carboxylic acid methyl ester

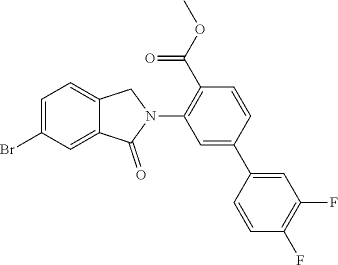 [SMILES: COC(=O)c1ccc(-c2ccc(F)c(F)c2)cc1N1Cc2ccc(Br)cc2C1=O]

94. This compound was prepared as described in the second step of synthetic procedure N from 3-({[4-bromo-2-(methoxycarbonyl)phenyl]methyl}amino)-3′,4′-difluorobiphenyl-4-carboxylic acid methyl ester (Intermediate 1c). MS m/z: (M+H)⁺ calculated for C₂₂H₁₆BrNO₃: 423.28. found 423.54. LC/MS retention time: 2.31 minutes.

### Intermediate 2a: 3-(6-Bromo-1-oxo-1,3-dihydroisoindol-2-yl)-3′,4′-difluorobiphenyl-4-carboxylic Acid

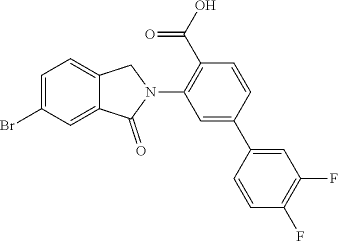 [SMILES: O=C(O)c1ccc(-c2ccc(F)c(F)c2)cc1N1Cc2ccc(Br)cc2C1=O]

95. This compound was prepared in crude form as described in synthetic procedure Q from 3-(6-bromo-1-oxo-1,3-dihydroisoindol-2-yl)-3′,4′-difluorobiphenyl-4-carboxylic acid methyl ester (Intermediate 2). Compound was used without additional purification.

### Intermediate 3: 4-(6-Bromo-1-oxo-1,3-dihydroisoindol-2-yl)biphenyl-3-carboxylic acid methyl ester

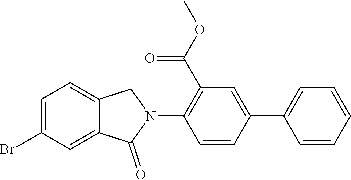 [SMILES: COC(=O)c1cc(-c2ccccc2)ccc1N1Cc2ccc(Br)cc2C1=O]

96. This compound was prepared as described in synthetic procedure N from methyl 5-bromo-2-(bromomethyl)benzoate (Intermediate 1a) and methyl 4-amino[1,1′-biphenyl]-3-carboxylate (Intermediate 12). MS m/z: (M+H)⁺ calculated for C₂₂H₁₆BrNO₃: 423.28. found 423.19. LC/MS retention time: 2.30 minutes.

### Intermediate 3a: 3-(6-Bromo-1-oxo-1,3-dihydroisoindol-2-yl)biphenyl-4-carboxylic acid methyl ester

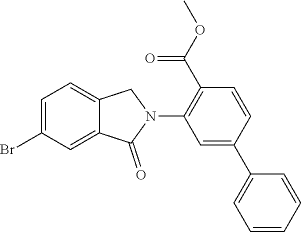 [SMILES: COC(=O)c1ccc(-c2ccccc2)cc1N1Cc2ccc(Br)cc2C1=O]

97. This compound was prepared as described in synthetic procedure N from methyl 5-bromo-2-(bromomethyl)benzoate (Intermediate 1a) and methyl 3-amino[1,1′-biphenyl]-4-carboxylate. MS m/z: (M+H)⁺ calculated for C₂₂H₁₆BrNO₃: 423.28. found 423.37. LC/MS retention time: 2.39 minutes.

### Intermediate 3b: 2-{4-[(Benzyloxy)carbonyl]-3′,4′-difluoro[1,1′-biphenyl]-3-yl}-3-oxo-2,3-dihydro-1H-isoindole-5-carboxylic Acid

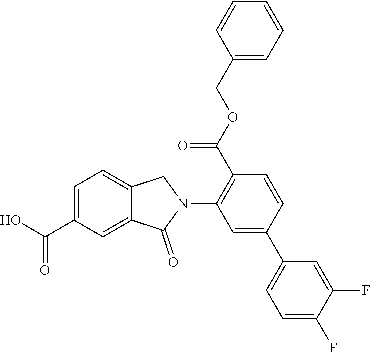 [SMILES: O=C(O)c1ccc2c(c1)C(=O)N(c1cc(-c3ccc(F)c(F)c3)ccc1C(=O)OCc1ccccc1)C2]

98. 2-{4-[(Benzyloxy)carbonyl]-3′,4′-difluoro[1,1′-biphenyl]-3-yl}-3-oxo-2,3-dihydro-1H-isoindole-5-carboxylic acid was prepared as described in synthetic procedure R from benzyl 3-(6-bromo-1-oxo-1,3-dihydroisoindol-2-yl)-3′,4′-difluorobiphenyl-4-carboxylate, which was prepared as described in synthetic procedure P route B from crude 3-(6-bromo-1-oxo-1,3-dihydroisoindol-2-yl)-3′,4′-difluorobiphenyl-4-carboxylic acid (Intermediate 2a) and phenylmethanol.

### Intermediate 4: 3-(6-amino-1-oxo-1,3-dihydroisoindol-2-yl)-3′,4′-difluorobiphenyl-4-carboxylic acid methyl ester

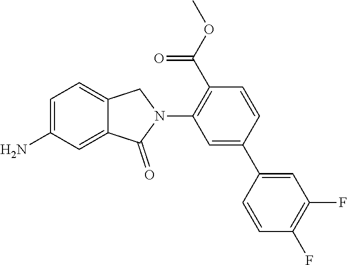 [SMILES: COC(=O)c1ccc(-c2ccc(F)c(F)c2)cc1N1Cc2ccc(N)cc2C1=O]

99. This compound was prepared as described in synthetic procedure U from 3-(6-bromo-1-oxo-1,3-dihydroisoindol-2-yl)-3′,4′-difluorobiphenyl-4-carboxylic acid methyl ester (Intermediate 2) and sodium azide. MS m/z: (M+H)⁺ calculated for C₂₂H₁₄BrF₂NO₃: 459.26. found 459.14. LC/MS retention time: 2.23 minutes.

### Intermediate 4a: 4-Ethynylbenzene-1,2-dicarboxylic Acid

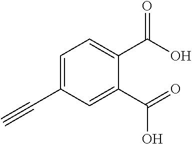

100. 4-Ethynylbenzene-1,2-dicarboxylic acid acid was prepared as described in synthetic procedure I from 4-bromophthalic anhydride and ethynyltrimethylsilane.

### Intermediate 5: 4-(1H-1,2,3-Triazol-4-yl)phthalic Acid

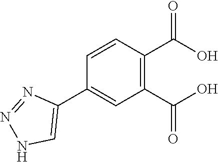 [SMILES: O=C(O)c1ccc(-c2c[nH]nn2)cc1C(=O)O]

101. This compound was prepared as described in synthetic procedure J from 4-ethynylbenzene-1,2-dicarboxylic acid (Intermediate 4a) and sodium azide.

### Intermediate 5a: 4-chloro-5-(1H-1,2,3-triazol-4-yl)phthalic Acid

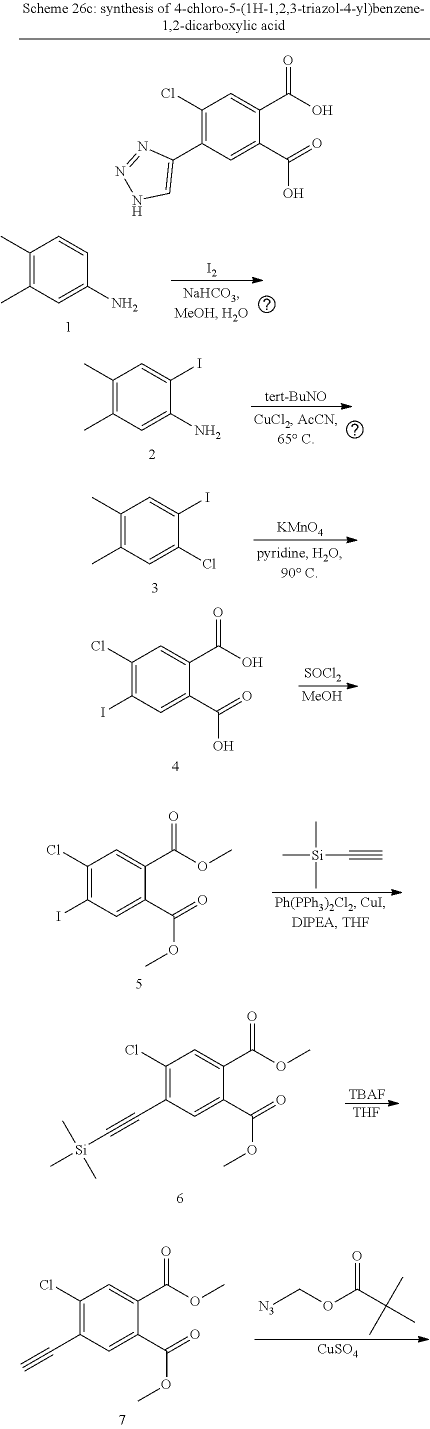

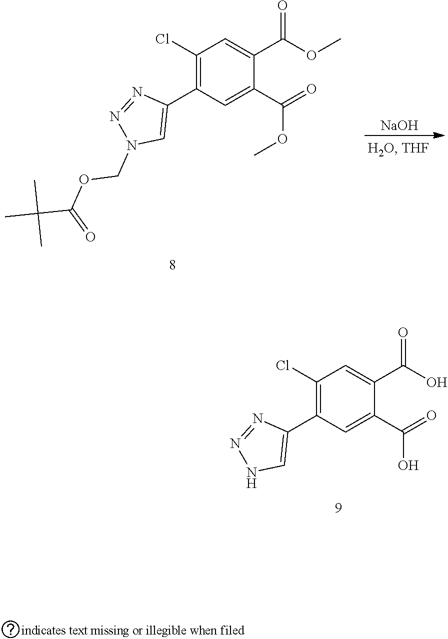 [SMILES: COC(=O)c1cc(Cl)c(-c2cn(COC(=O)C(C)(C)C)nn2)cc1C(=O)OC ; "NaOH" ; O=C(O)c1cc(Cl)c(-c2c[nH]nn2)cc1C(=O)O]

Step 1: synthesis of 2-iodo-4,5-dimethylaniline 102. To a stirred suspension of 3,4-dimethylaniline (Compound 1 in Scheme 26c, 12 g, 99 mmol), NaHCO₃ (16.6 g, 198 mmol) in methanol (100 mL) and water (50 mL) was added iodine (25.1 g, 99 mmol) in portions. After stirred at room temperature overnight, the reaction mixture was quenched with sat. Na₂SO₃, then was extracted with ethyl acetate (200 mL×2), the combined organic layers were washed with brine, dried over Na₂SO₄ and concentrated, the residue was purified by flash chromatography (silica gel, 20% ethyl acetate in petroleum ether) to provide 2-iodo-4,5-dimethylbenzenamine (Compound 2, 20 g, 82%) as a liquid. ESI-MS m/z calc. 247.07, found 248.34 (M+H)⁺.

Step 2: synthesis of 1-chloro-2-iodo-4,5-dimethylbenzene

103. At room temperature, to the suspension of 2-iodo-4,5-dimethylaniline (Compound 2 in scheme 26c, 16.5 g, 66.8 mmol), CuCl₂ (10.8 g, 80.2 mmol) in acetonitrile (200 mL) was added tert-butyl nitrite (10.3 g, 100.2 mmol) dropwise. The resulting mixture was heated to 65° C. for 30 minutes. After cooled down to room temperature, the reaction was poured into ice water and extracted with ethyl acetate (250 mL×3), the combined organic layers were washed with brine and dried over Na₂SO₄, filtered and concentrated, the residue was purified by flash chromatography (silica gel, 0-2% ethyl acetate in petroleum ether) to provide 1-chloro-2-iodo-4,5-dimethylbenzene (Compound 3 in scheme 26c, 11 g, 62%) as a liquid.

Step 3: synthesis of 4-chloro-5-iodophthalic acid

104. At room temperature, to a solution of 1-chloro-2-iodo-4,5-dimethylbenzene (Compound 3 in Scheme 26c, 11 g, 41.3 mmol) in pyridine (100 mL) and water (150 mL) was added KMnO₄ (98 g, 620 mmol). The resulting mixture was heated to 90° C. overnight. The hot mixture was filtered and the residue was washed with aqueous potassium hydroxide solution (1 M, 200 mL), the filtrate was acidified with conc. HCl to pH 1-2. The mixture was filtered then the desired solid was collected and dried in vacuum to provide 4-chloro-5-iodophthalic acid (Compound 4 in Scheme 26c) (10.8 g, 80%) as a white solid which was used directly in the next step. LC-MS ESI (m/z): calc. 326.47, found 327.18/329.20 M/(M+2).

Step 4: synthesis of dimethyl 4-chloro-5-iodophthalate

105. At 0° C., to a solution of 4-chloro-5-iodophthalic acid (Compound 4 in Scheme 26c, 10.8 g, 33.1 mmol) in methanol (150 mL) was added SOCl₂ (24 mL, 331 mmol) dropwise. The resulting mixture was heated to 60° C. overnight. Solvent was removed under vacuum, the residue was redissolved in ethyl acetate (100 mL), and then was washed with brine, dried over Na₂SO₄, filtered and concentrated, the residue was purified by flash chromotography (silica gel, 25% ethyl acetate in petroleum ether) to provide dimethyl 4-chloro-5-iodophthalate (Compound 5, 9.5 g, 81%) as a light yellow liquid. LC-MS ESI (m/z): calc. 354.53, found 355.36/357.37 M/(M+2).

Step 5: synthesis of dimethyl 4-chloro-5-[(trimethylsilyl)ethynyl]phthalate

106. At room temperature, to a mixture of dimethyl 4-chloro-5-iodophthalate (Compound 5 in Scheme 26c, 9.5 g, 26.8 mmol), Pd(PPh₃)₂Cl₂ (3.76 g, 5.36 mmol), CuI (510 mg, 2.68 mmol) and DIPEA (14 mL, 80.4 mmol) in THF (40 mL) was added ethynyltrimethylsilane (3.9 g, 40.2 mmol) dropwise, the resulting mixture was stirred for 30 minutes. After removed the solvent, the residue was purified by flash chromatography (silica gel, 10% ethyl acetate in petroleum ether) to provide dimethyl 4-chloro-5-[(trimethylsilyl)ethynyl]phthalate (Compound 6 in Scheme 26c, 4.55 g, 52%) as a light yellow solide. LC-MS ESI (m/z): calc. 324.83, found 325.39/327.40 M/(M+2).

Step 6: synthesis of dimethyl 4-chloro-5-ethynylphthalate

107. At room temperature, to a solution of 4-chloro-5-[(trimethylsilyl)ethynyl]phthalate (Compound 6 in Scheme 26c, 4.55 g, 14 mmol) in THF (10 mL) was added TBAF (28 mL, 1 M in THF), the resulting mixture was stirred for 20 minutes. After poured into ethyl acetate/water mixture (40 mL/40 mL), the organic layer was separated and washed with sat. NH₄Cl (30 mL) and brine, dried over Na₂SO₄, filtered and concentrated, the residue was purified by flash chromatography (silica gel, 25% ethyl acetate in petroleum ether) to provide dimethyl 4-chloro-5-ethynylphthalate (Compound 7 in Scheme 26c, 2.05 g, 58%) as solid. LC-MS ESI (m/z): calc. 252.65, found 253.27/255.22 M/(M+2).

Step 7: synthesis of dimethyl 4-chloro-5-(1-(pivaloyloxymethyl)-1H-1,2,3-triazol-4-yl)phthalate 108. To the mixture of dimethyl 4-chloro-5-ethynylphthalate (Compound 7 in Scheme 26c, 2.05 g, 8.1 mmol), CuSO₄ (259 mg, 1.62 mmol), sodium ascorbate (321 mg, 1.62 mmol) in tert-butanol (15 mL) and water (15 mL) was added azidomethyl pivalate (1.9 g, 12.15 mmol), the resulting mixture was stirred at room temperature overnight. After being poured into ethyl acetate/water mixture (25 mL/25 mL), the ethyl acetate layer was separated and the aqueous layer was extracted with ethyl acetate (20 mL×2), the combined organic phases were washed with brine, dried over Na₂SO₄, filtered and concentrated. The residue was purified by flash chromatography (silica gel, 25% ethyl acetate in petroleum ether) to provide dimethyl 4-chloro-5-(1-(pivaloyloxymethyl)-1H-1,2,3-triazol-4-yl)phthalate (Compound 8 in Scheme 26c, 1.6 g, 48%) as a white solid. LC-MS ESI (m/z): calc. 409.82, found 410.40/412.40 M/(M+2).

Step 8: synthesis of 4-chloro-5-(1H-1,2,3-triazol-4-yl)phthalic acid

109. At 0° C., to a stirred solution of dimethyl 4-chloro-5-(1-(pivaloyloxymethyl)-1H-1,2,3-triazol-4-yl)phthalate (Compound 8 in Scheme 26c, 1.6 g, 3.9 mmol) in THF (10 mL) was added LiOH (468 mg, 19.5 mmol) in water (10 mL). After stirred at RT for 1 h, the reaction mixture was acidified with 1 M HCl to pH 7, solvent was removed under vacuum, the residue was purified by reverse phase HPLC (C18, 5-40% acetonitrile in H₂O with 0.1% formic acid) to provide 4-chloro-5-(1H-1,2,3-triazol-4-yl)phthalic acid (Compound 9 in Scheme 26c, 506 mg, 48%) as a white solid. LC-MS ESI (m/z): calc. 267.62, found 268.32/270.29 M/(M+2). ¹H NMR (400 MHz, D20) δ 8.43 (s, 1H), 8.18 (s, 1H), 7.86 (s, 1H).

### Intermediate 5b: 5-hydroxybenzene-1,2,4-tricarboxylic Acid

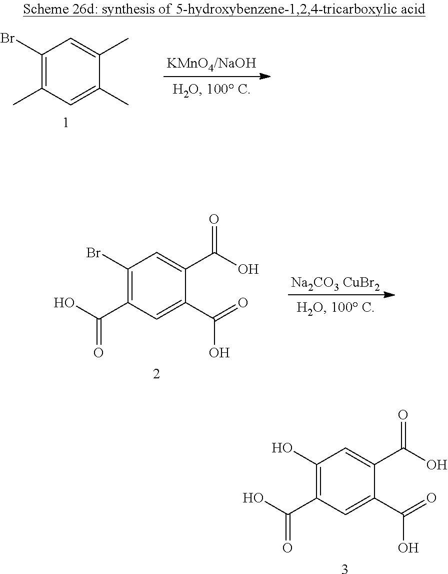

Step 1: synthesis of 5-bromobenzene-1,2,4-tricarboxylic Acid

An oven-dried 500 mL Schlenk flask equipped with a magnetic stir bar was charged with 1-bromo-2,4,5-trimethylbenzene (Compound 1 in Scheme 26d, 6.00 g, 30.1 mmol), sodium hydroxide (1.50 g, 37.5 mmol), potassium permanganate (31.5 g, 199 mmol, 6.6 equiv) and 150 mL of deionized water. The flask was fitted with a reflux condenser and then submerged in an oil bath and the reaction mixture was stirred at reflux overnight. 15 mL of methanol was added to reduce excess KMnO₄ and the hot solution was filtered through celite. The manganese dioxide was washed 3-4 times with 20 mL of boiling water and each wash was collected and combined. Concentrated hydrochloric acid was added to the aqueous solution until the pH was acidic. The solution was extracted with diethyl ether (5×100 mL). The organic extracts were combined, dried using Na₂SO₄ and filtered; the organic solution was concentrated by rotary evaporation to afford 5-bromobenzene-1,2,4-tricarboxylic acid as a white powder (Compound 2 in Scheme 26c, 4.8 g, 55%).

Step 2: synthesis of 5-hydroxybenzene-1,2,4-tricarboxylic Acid

Under nitrogen, 5-bromobenzene-1,2,4-tricarboxylic acid (Compound 2 in Scheme 26d, 80 mg, 0.277 mmol) was combined with 2.2 mL of H₂O, 265 mg (2.49 mmol) of Na₂CO₃, 2.2 mg of CuBr₂ and 2.8 mg of trans-N,N′-dimethylcyclohexane-1,2-diamine was then added. This reaction mixture was stirred at 80° C. under nitrogen and stirred for 2 h at 80° C. After cooling to 25° C., the reaction mixture was acidified with 15% HCl, producing a white precipitate. The white precipitate was filtered and washed with water. After drying, a total of 58.5 mg 5-hydroxybenzene-1,2,4-tricarboxylic acid was collected. (Compound 3 in Scheme 26d, 0.26 mmol, 84% yield)

### Intermediate 6: Methyl 2-amino-5-(1H-imidazol-4-yl)benzoate

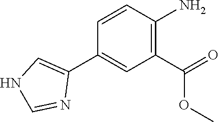 [SMILES: COC(=O)c1cc(-c2c[nH]cn2)ccc1N]

Step 1: Methyl 2-amino-5-(pinacolboranyl)benzoate

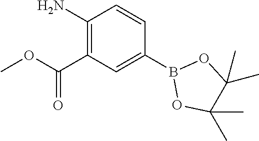

110. Methyl 2-amino-5-(pinacolboranyl)benzoate was prepared as described in synthetic procedure C from methyl 2-amino-5-bromobenzoate and bis(pinacolato)diboron.

Step 2: Methyl 2-amino-5-(1H-imidazol-4-yl)benzoate

111. This compound was prepared as described in synthetic procedure D followed by synthetic procedure F from methyl 2-amino-5-(pinacolboranyl)benzoate and 4-iodo-1-(triphenylmethyl)-1H-imidazole.

### Intermediate 6a: 2-{5-[(Benzyloxy)carbonyl]-1,3-dioxoisoindolin-2-yl}-5-(1H-imidazol-4-yl)benzoic acid

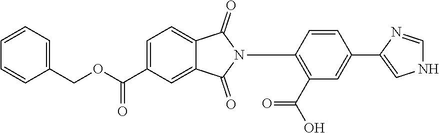 [SMILES: O=C(OCc1ccccc1)c1ccc2c(c1)C(=O)N(Cc1ccc(-c3c[nH]cn3)cc1C(=O)O)C2=O]

112. This compound was prepared as described in synthetic procedure K1 from

benzyl 1,3-dioxo-1,3-dihydro-2-benzofuran-5-carboxylate (chemical building block, was prepared from phenylmethanol and 1,3-dioxo-1,3-dihydro-2-benzofuran-5-carbonyl chloride by the method of the patent WO 2003074516) and methyl 2-amino-5-(1H-imidazol-4-yl)benzoate (Intermediate 6).

### Intermediate 7: 2-Amino-4-(4-methoxyphenyl)-5-methylthiophene-3-carbonitrile

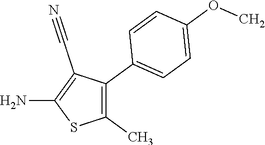 [SMILES: COc1ccc(-c2c(C)sc(N)c2C#N)cc1]

113. 2-Amino-4-(4-methoxyphenyl)-5-methylthiophene-3-carbonitrile was prepared as described in synthetic procedure G from sulfur, malononitrile and 1-(4-methoxyphenyl)propan-1-one.

### Intermediate 7a: 2-Amino-4,5-diphenylthiophene-3-carbonitrile

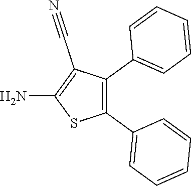 [SMILES: N#Cc1c(N)sc(-c2ccccc2)c1-c1ccccc1]

114. 2-Amino-4,5-diphenylthiophene-3-carbonitrile was prepared as described in synthetic procedure G from sulfur, malononitrile and 1,2-diphenylethan-1-one.

### Intermediate 8: 2-(6-Bromo-1-oxo-1,3-dihydroisoindol-2-yl)-4-(4-methoxyphenyl)-5-methylthiophene-3-carbonitrile

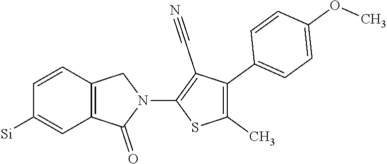

115. 2-(6-Bromo-1-oxo-1,3-dihydroisoindol-2-yl)-4-(4-methoxyphenyl)-5-methylthiophene-3-carbonitrile was prepared as described in the synthetic procedure O from 2-amino-4-(4-methoxyphenyl)-5-methylthiophene-3-carbonitrile (Intermediate 7) and methyl 5-bromo-2-(bromomethyl)benzoate (Intermediate 1a).

### Intermediate 9: 2-Amino-4-(2,4-difluorophenyl)-5-methylpyridine

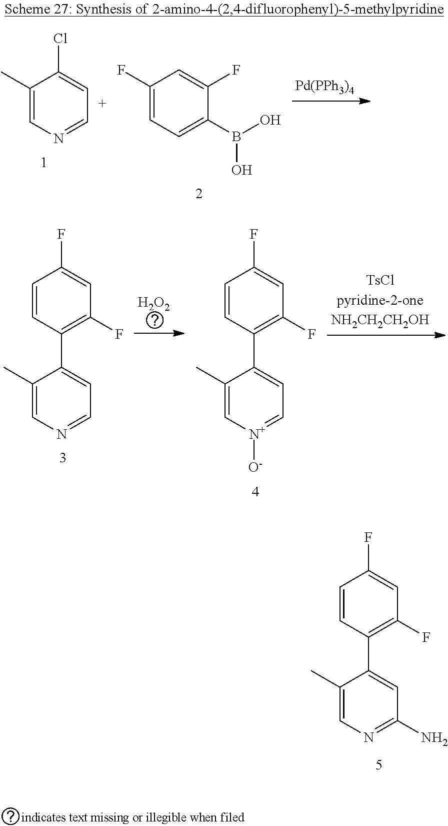

Step 1: synthesis of 3-methyl-4-(2,4-difluorophenyl)pyridine 116. 4-Chloro-3-methylpyridine (Compound 1 on FIG. 27, 2.00 g, 15.7 mmol), 2,4-difluorophenylboronic acid (Compound 2 in Scheme 27, 3.53 g, 24.4 mmol), a 2M aqueous solution of potassium carbonate (31.4 mL, 62.8 mmol) and Pd(PPh₃)₄ (0.906 g, 0.784 mmol) were suspended in 1,2-dimethoxyethane (DME, 150 mL). The resulting mixture was stirred and heated to 80° C. for 60 hours. The crude reaction mixture was cooled to room temperature and then the layers were separated. The organic layer was evaporated to dryness and then purified on 250 g of silica gel utilizing a gradient of 0-70% ethyl acetate in hexane to yield the pure product as a pale yellow oil (Compound 3 in Scheme 27, 2.29 g, 11.1 mmol, 71%).

Step 2: synthesis of 3-methyl-4-(2,4-difluorophenyl)pyridine 1-oxide

117. 3-Methyl-4-(2,4-difluorophenyl)pyridine (Compound 3 in Scheme 27, 2.29 g, 11.1 mmol) was dissolved in a mixture of dichloromethane (4.0 mL) and 30% hydrogen peroxide (1.95 mL). Methyltrioxorhenium (VII) (11.5 mg, 4.6 mmol) was added and the reaction mixture was stirred vigorously for 5 hours. The layers were then separated and the organic layer was treated with sodium sulfite, and then dried over sodium sulfate. The crude product filtered, evaporated to dryness, and used without further purification. ESI-MS m/z calc. 221.2, found 222.1 (M+H)⁺. Retention time: 1.22 minutes.

Step 3: synthesis of 2-amino-4-(2,4-difluorophenyl)-5-methylpyridine

118. 3-Methyl-4-(2,4-difluorophenyl)pyridine 1-oxide (Compound 4 in Scheme 27, 0.362 g, 1.64 mmol) was dissolved in a mixture of pyridine (0.5 mL) and acetonitrile (15 mL) under an atmosphere of argon. 4-Toluenesulfonyl chloride (TsCl, 0.406 g, 2.13 mmol) was added and the reaction mixture was stirred at 75° C. for 3 days. Ethanolamine (7 mL) was then added and the reaction mixture was allowed to stir for 5 minutes at room temperature. The crude product was partitioned between chloroform and a saturated aqueous solution of sodium bicarbonate. The layers were separated and the organic layer was washed with a brine. The organic layer was dried over sodium sulfate and then purified on 60 g of silica gel utilizing a gradient of 0-100% ethyl acetate in hexane to yield the pure title product (Compound 5 in Scheme 27, 0.13 g, 0.591 mmol, 36.1%). ESI-MS m/z calc. 220.2, found 221.1 (M+H)⁺. Retention time of 1.09 minutes.

### Intermediate 10: Methyl 4-amino-6-methoxy[1,1′-biphenyl]-3-carboxylate

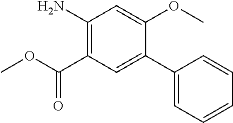 [SMILES: COC(=O)c1cc(-c2ccccc2)c(OC)cc1N]

119. Methyl 4-amino-6-methoxy[1,1′-biphenyl]-3-carboxylate was prepared using the process described in synthetic procedure A from phenylboronic acid and substituting methyl 2-amino-5-bromo-4-methoxybenzoate for methyl bromoanthranilate.

### Intermediate 11a: N-(Methanesulfonyl)-1,3-dioxo-1,3-dihydro-2-benzofuran-5-carboxamide

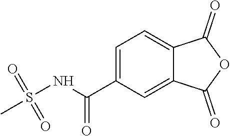 [SMILES: CS(=O)(=O)NC(=O)c1ccc2c(c1)C(=O)OC2=O]

120. N-(Methanesulfonyl)-1,3-dioxo-1,3-dihydro-2-benzofuran-5-carboxamide was obtained in crude form as described in synthetic procedure J3 from methanesulfonamide and 1,3-dioxo-1,3-dihydro-2-benzofuran-5-carbonyl chloride.

### Intermediate 11 b: N-(Butane-1-sulfonyl)-1,3-dioxo-1,3-dihydro-2-benzofuran-5-carboxamide

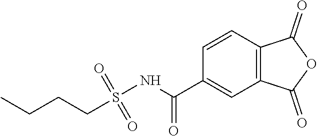 [SMILES: CCCCS(=O)(=O)NC(=O)c1ccc2c(c1)C(=O)OC2=O]

N-(Butane-1-sulfonyl)-1,3-dioxo-1,3-dihydro-2-benzofuran-5-carboxamide was obtained in crude form as described in synthetic procedure J3 from butane-1-sulfonamide and 1,3-dioxo-1,3-dihydro-2-benzofuran-5-carbonyl chloride.

### Intermediate 11c: N-(Benzenesulfonyl)-1,3-dioxo-1,3-dihydro-2-benzofuran-5-carboxamide

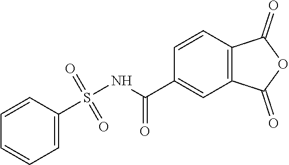 [SMILES: O=C(NS(=O)(=O)c1ccccc1)c1ccc2c(c1)C(=O)OC2=O]

121. N-(Benzenesulfonyl)-1,3-dioxo-1,3-dihydro-2-benzofuran-5-carboxamide was obtained in crude form as described in synthetic procedure J3 from benzenesulfonamide and 1,3-dioxo-1,3-dihydro-2-benzofuran-5-carbonyl chloride.

### Intermediate 12: Methyl 4-amino[1,1′-biphenyl]-3-carboxylate

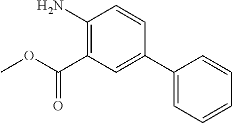 [SMILES: COC(=O)c1cc(-c2ccccc2)ccc1N]

122. Methyl 4-amino[1,1′-biphenyl]-3-carboxylate was prepared as described in synthetic procedure A from methyl 2-amino-5-bromobenzoate and phenylboronic acid.

### Intermediate 13: 4-Amino[1,1′-biphenyl]-3-carboxylic Acid

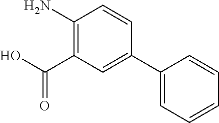 [SMILES: Nc1ccc(-c2ccccc2)cc1C(=O)O]

123. 4-Amino[1,1′-biphenyl]-3-carboxylic acid was prepared as described in synthetic procedure B from methyl 4-amino[1,1′-biphenyl]-3-carboxylate (Intermediate 12).

### Intermediate 14: Methyl 3-amino-4′-fluoro[1,1′-biphenyl]-4-carboxylate

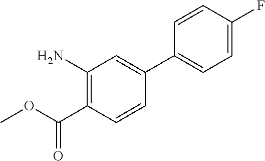 [SMILES: COC(=O)c1ccc(-c2ccc(F)cc2)cc1N]

124. Methyl 3-amino-4′-fluoro[1,1′-biphenyl]-4-carboxylate was prepared as described in synthetic procedure A from methyl 2-amino-4-bromobenzoate and 4-fluorophenylboronic acid.

### Intermediate 15: 3-Amino-3′-fluoro[1,1′-biphenyl]-4-carboxylic Acid

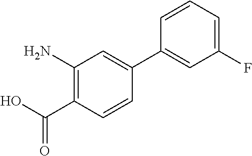 [SMILES: Nc1cc(-c2cccc(F)c2)ccc1C(=O)O]

125. 3-Amino-3′-fluoro[1,1′-biphenyl]-4-carboxylic acid was prepared as described in synthetic procedure A followed by synthetic procedure B from methyl 2-amino-4-bromobenzoate and 3-fluorophenylboronic acid.

### Intermediate 16: 3-Amino-2′,4′-difluoro[1,1′-biphenyl]-4-carboxylic Acid

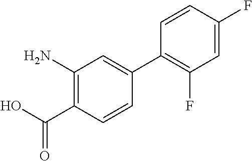 [SMILES: Nc1cc(-c2ccc(F)cc2F)ccc1C(=O)O]

126. 3-Amino-2′,4′-difluoro[1,1′-biphenyl]-4-carboxylic acid was prepared as described in synthetic procedure A followed by synthetic procedure B from methyl 2-amino-4-bromobenzoate and 2,4-difluorophenylboronic acid.

### Intermediate 17: 2-Amino-4-(pyridin-3-yl)benzoic acid

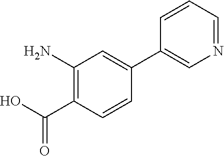 [SMILES: Nc1cc(-c2cccnc2)ccc1C(=O)O]

127. 2-Amino-4-(pyridin-3-yl)benzoic acid was prepared as described in synthetic procedure A followed by synthetic procedure E from methyl 2-amino-4-bromobenzoate and pyridine-3-boronic acid.

### Intermediate 18: 3-(1H-tetrazol-5-yl)[1,1′-biphenyl]-4-amine

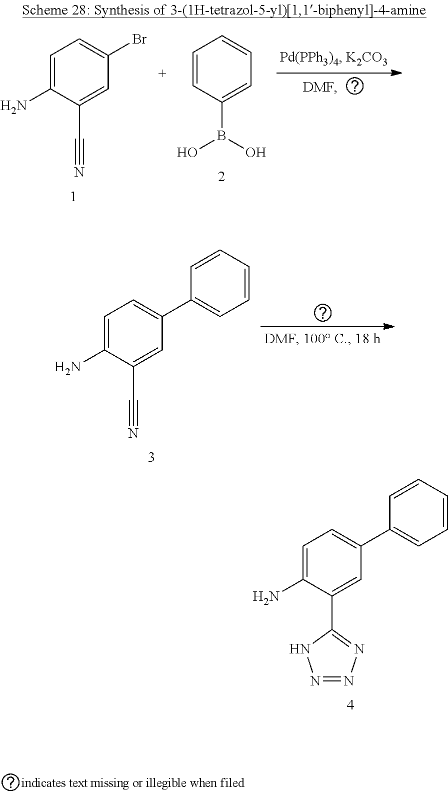

Step 1: synthesis of 4-amino[1,1′-biphenyl]-3-carbonitrile

To a mixed solution of 2-amino-5-bromobenzonitrile (Compound 1 on scheme 28, 0.05 g, 5.33 mmol) and phenylboronic acid (Compound 2 on scheme 28, 974 mg, 7.99 mmol) in DMF (24 ml) was added Pd(PPh₃)₄ (280 mg, 0.266 mmol) followed by aqueous K₂CO₃ (1 M, 8 ml). The reaction mixture was stirred 95° C. for 8 h. After reaction finished, the reaction mixture was poured into H₂O (250 ml), and extracted with ethyl acetate (100 ml×3). The combined organic layers were washed with H₂O (100 ml×2), dried over Na₂SO₄, filtered, concentrated, purified by chromatography (silica gel, hexane/ethyl acetate=100/0-70/30) to give 2-amino-5-phenylbenzonitrile. (Compound 3 on scheme 28, 858 mg, 83%) MS (m/z): 195.0 (M+H)⁺.

Step 2: synthesis of 3-(1H-tetrazol-5-yl)[1,1′-biphenyl]-4-amine

To a mixed solution of 2-amino-5-phenylbenzonitrile (Compound 3 on scheme 28, 117 mg, 0.6 mmol) and triethylamine hydrochloride (290 mg, 2.1 mmol) in DMF (4.5 mL) was added sodium azide (137 mg, 2.1 mmol). The reaction mixture was stirred at 100° C. for 18 h. After reaction finished, the reaction mixture was poured into H₂O (20 ml), and extracted with ethyl acetate (15 mL×3). The combined organic layers were washed with H₂O (20 ml), dried over Na₂SO₄, filtered, concentrated to give crude 3-(1H-tetrazol-5-yl)[1,1′-biphenyl]-4-amine (Compound 4 on scheme 28). MS (m/z): 238.0 (M+H)⁺.

### Intermediate 19: 4-(1H-tetrazol-5-yl)[1,1′-biphenyl]-3-amine

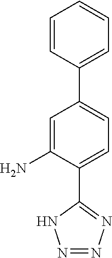 [SMILES: Nc1cc(-c2ccccc2)ccc1-c1nnn[nH]1]

128. Crude 4-(1H-tetrazol-5-yl)[1,1′-biphenyl]-3-amine was prepared as described in synthetic procedure for Intermediate 18 from 2-amino-4-bromobenzonitrile, phenylboronic acid and sodium azide.

### Example 1: 2-(2-Methoxybiphenyl-4-yl)-1,3-dioxo-2,3-dihydro-1H-isoindole-5-carboxylic Acid

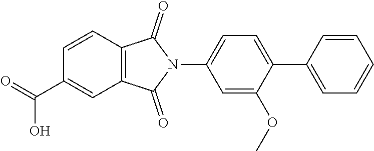 [SMILES: COc1cc(N2C(=O)c3ccc(C(=O)O)cc3C2=O)ccc1-c1ccccc1]

129. 2-(2-Methoxybiphenyl-4-yl)-1,3-dioxo-2,3-dihydro-1H-isoindole-5-carboxylic acid was prepared as described in synthetic procedure K starting from trimellitic anhydride and 2-methoxy-[1,1′-biphenyl]-4-amine; MS m/z: (M+H)⁺ calculated for C₂₂H₁₅NO₅: 374.36. found 374.14. LC/MS retention time: 2.53 minutes.

### Example 2: 1,3-Dioxo-2-[3-(1H-tetrazol-5-yl)-1,1′-biphenyl-4-yl]-2,3-dihydro-1H-isoindole-5-carboxylic Acid

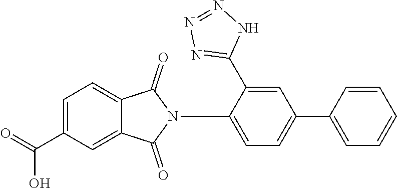 [SMILES: O=C(O)c1ccc2c(c1)C(=O)N(c1ccc(-c3ccccc3)cc1-c1nnn[nH]1)C2=O]

130. 1,3-Dioxo-2-[3-(1H-tetrazol-5-yl)-1,1′-biphenyl-4-yl]-2,3-dihydro-1H-isoindole-5-carboxylic acid was prepared as described in synthetic procedure K from trimellitic anhydride and crude 3-(1H-tetrazol-5-yl)[1,1′-biphenyl]-4-amine (Intermediate 18); MS m/z: (M+H)⁺ calculated for C₂₂H₁₃N₅O₄: 412.38. found 412.26. LC/MS retention time: 2.19 minutes.

### Example 3: 2-(4-Hydroxy[1,1′-biphenyl]-3-yl)-1,3-dioxo-2,3-dihydro-1H-isoindole-5-carboxylic Acid

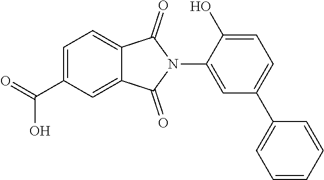 [SMILES: O=C(O)c1ccc2c(c1)C(=O)N(c1cc(-c3ccccc3)ccc1O)C2=O]

131. 2-(4-Hydroxy[1,1′-biphenyl]-3-yl)-1,3-dioxo-2,3-dihydro-1H-isoindole-5-carboxylic acid was prepared as described in synthetic procedure K from trimellitic anhydride and 3-amino-[1,1′-biphenyl]-4-ol; MS m/z: (M+H)⁺ calculated for C₂₁H₁₃NO₅: 360.34. found 360.1. LC/MS retention time: 2.01 minutes.

### Example 4: 2-(3-Hydroxymethylbiphenyl-4-yl)-1,3-dioxo-2,3-dihydro-1H-isoindole-5-carboxylic Acid

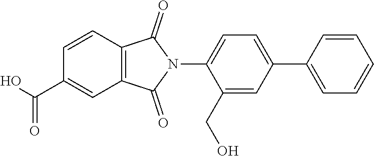 [SMILES: O=C(O)c1ccc2c(c1)C(=O)N(c1ccc(-c3ccccc3)cc1CO)C2=O]

132. 2-(3-Hydroxymethylbiphenyl-4-yl)-1,3-dioxo-2,3-dihydro-1H-isoindole-5-carboxylic acid was prepared as described in synthetic procedure K starting from trimellitic anhydride and (4-amino[1,1′-biphenyl]-3-yl)methanol (was prepared from 4-amino[1,1′-biphenyl]-3-carboxylic acid (Intermediate 13) as described in J. Org. Chem., 2008, 73(11), 4252-4255); MS m/z: (M+H)⁺ calculated for C₂₁H₁₃NO₅: 374.37. found 374.37. LC/MS retention time: 2.27 minutes.

### Example 5: 2-(3-Methoxycarbonylbiphenyl-4-yl)-1,3-dioxo-2,3-dihydro-1H-isoindole-5-carboxylic Acid methyl ester

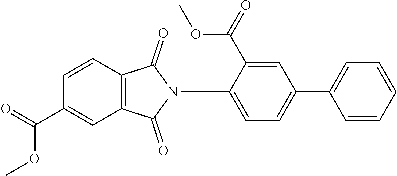 [SMILES: COC(=O)c1ccc2c(c1)C(=O)N(c1ccc(-c3ccccc3)cc1C(=O)OC)C2=O]

133. 2-(3-Methoxycarbonylbiphenyl-4-yl)-1,3-dioxo-2,3-dihydro-1H-isoindole-5-carboxylic acid methyl ester was prepared as described in synthetic procedure AD from 2-(3-methoxycarbonylbiphenyl-4-yl)-1,3-dioxo-2,3-dihydro-1H-isoindole-5-carboxylic acid (see Example 12) and methanol; MS m/z: (M+H)⁺ calculated for C₂₄H₁₇NO₆: 416.41. found 416.16. LC/MS retention time: 2.81 minutes.

### Example 6: 2-[3-Cyano-4-(4-methoxyphenyl)-5-methylthiophen-2-yl]-1,3-dioxo-2,3-dihydro-1H-isoindole-5-carboxylic Acid

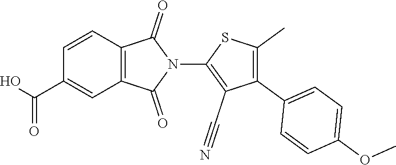 [SMILES: COc1ccc(-c2c(C)sc(N3C(=O)c4ccc(C(=O)O)cc4C3=O)c2C#N)cc1]

134. 2-[3-Cyano-4-(4-methoxyphenyl)-5-methylthiophen-2-yl]-1,3-dioxo-2,3-dihydro-1H-isoindole-5-carboxylic acid was prepared as described in synthetic procedure K starting from trimellitic anhydride and 2-amino-4-(4-methoxyphenyl)-5-methylthiophene-3-carbonitrile (Intermediate 7); MS m/z: (M+H)⁺ calculated for C₂₂H₁₄N₂O₅S: 419.43. found 419.13. LC/MS retention time: 2.87 minutes.

### Example 7: 2-[3-Cyano-4-(4-methoxyphenyl)-5-methylthiophen-2-yl]-1,3-dioxo-2,3-dihydro-1H-isoindole-5-carboxylic Acid ethyl ester

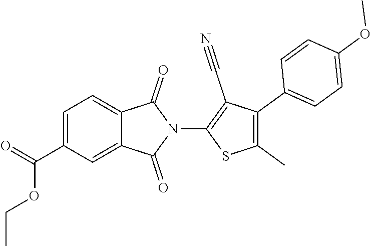 [SMILES: CCOC(=O)c1ccc2c(c1)C(=O)N(c1sc(C)c(-c3ccc(OC)cc3)c1C#N)C2=O]

135. 2-[3-Cyano-4-(4-methoxyphenyl)-5-methylthiophen-2-yl]-1,3-dioxo-2,3-dihydro-1H-isoindole-5-carboxylic acid ethyl ester was prepared as described in synthetic procedure AD from 2-[3-cyano-4-(4-methoxyphenyl)-5-methylthiophen-2-yl]-1,3-dioxo-2,3-dihydro-1H-isoindole-5-carboxylic acid (see Example 6) and ethanol; MS m/z: (M+H)⁺ calculated for C₂₄H₁₈N₂O₅S: 447.49. found 447.08. LC/MS retention time: 3.00 minutes.

### Example 8: 2-[3-Cyano-4-(4-methoxyphenyl)-5-methylthiophen-2-yl]-1,3-dioxo-2,3-dihydro-1H-P isoindole-5-carboxylic acid butyl ester

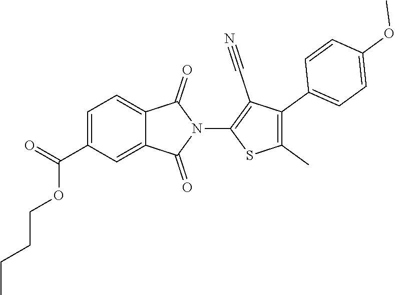 [SMILES: CCCCOC(=O)c1ccc2c(c1)C(=O)N(c1sc(C)c(-c3ccc(OC)cc3)c1C#N)C2=O]

136. 2-[3-Cyano-4-(4-methoxyphenyl)-5-methylthiophen-2-yl]-1,3-dioxo-2,3-dihydro-1H-isoindole-5-carboxylic acid butyl ester was prepared as described in synthetic procedure AD from 2-[3-cyano-4-(4-methoxyphenyl)-5-methylthiophen-2-yl]-1,3-dioxo-2,3-dihydro-1H-isoindole-5-carboxylic acid (see Example 6) and n-butanol; MS m/z: (M+H)⁺ calculated for C₂₆H₂₂N₂O₅S: 475.54. found 474.99. LC/MS retention time: 3.27 minutes.

### Example 9: 1,3-Dioxo-2-[3-(1H-tetrazol-5-yl)biphenyl-4-yl]-2,3-dihydro-1H-isoindole-5-carboxylic Acid ethyl ester

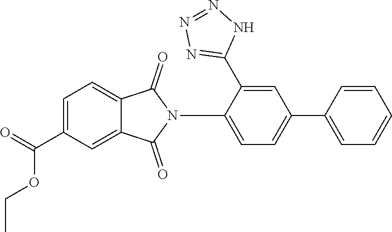 [SMILES: CCOC(=O)c1ccc2c(c1)C(=O)N(c1ccc(-c3ccccc3)cc1-c1nnn[nH]1)C2=O]

137. 1,3-Dioxo-2-[3-(1H-tetrazol-5-yl)biphenyl-4-yl]-2,3-dihydro-1H-isoindole-5-carboxylic acid ethyl ester was prepared as described in synthetic procedure AD from 1,3-dioxo-2-[3-(1H-tetrazol-5-yl)-1,1′-biphenyl-4-yl]-2,3-dihydro-1H-isoindole-5-carboxylic acid (see Example 2) and ethanol; MS m/z: (M+H)⁺ calculated for C₂₄H₁₇N₅O₄: 440.43. found 440.18. LC/MS retention time: 2.66 minutes.

### Example 10: 2-(4-Carboxybiphenyl-3-yl)-1,3-dioxo-2,3-dihydro-1H-isoindole-5-carboxylic Acid

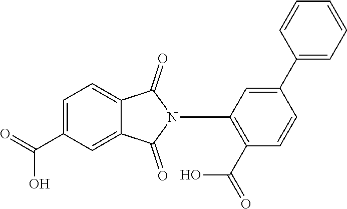 [SMILES: O=C(O)c1ccc2c(c1)C(=O)N(c1cc(-c3ccccc3)ccc1C(=O)O)C2=O]

138. 2-(4-Carboxybiphenyl-3-yl)-1,3-dioxo-2,3-dihydro-1H-isoindole-5-carboxylic acid was prepared as described in synthetic procedure K starting from trimellitic anhydride and 3-amino[1,1′-biphenyl]-4-carboxylic acid; MS m/z: (M+H)⁺ calculated for C₂₂H₁₃NO₆: 388.35. found 387.95; LC/MS retention time: 2.23 minutes.

### Example 11: 2-(4-Hydroxymethylbiphenyl-3-yl)-1,3-dioxo-2,3-dihydro-1H-isoindole-5-carboxylic Acid

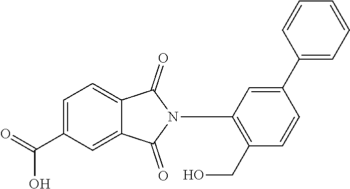 [SMILES: O=C(O)c1ccc2c(c1)C(=O)N(c1cc(-c3ccccc3)ccc1CO)C2=O]

139. 2-(4-Hydroxymethylbiphenyl-3-yl)-1,3-dioxo-2,3-dihydro-1H-isoindole-5-carboxylic acid was prepared as described in synthetic procedure K from trimellitic anhydride and (3-amino[1,1′-biphenyl]-4-yl)methanol (was prepared from 3-amino[1,1′-biphenyl]-4-carboxylic acid as described in J. Org. Chem., 2008, 73(11), 4252-4255); MS m/z: (M+H)⁺ calculated for C₂₂H₁₅NO₅: 374.37. found 374.44. LC/MS retention time: 2.38 minutes.

### Example 12: 2-(3-Methoxycarbonylbiphenyl-4-yl)-1,3-dioxo-2,3-dihydro-1H-isoindole-5-carboxylic Acid

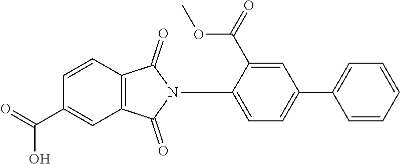 [SMILES: COC(=O)c1cc(-c2ccccc2)ccc1N1C(=O)c2ccc(C(=O)O)cc2C1=O]

140. 2-(3-Methoxycarbonylbiphenyl-4-yl)-1,3-dioxo-2,3-dihydro-1H-isoindole-5-carboxylic acid was prepared as described in synthetic procedure K starting from trimellitic anhydride and methyl 4-amino[1,1′-biphenyl]-3-carboxylate (Intermediate 12); MS m/z: (M+H)⁺ calculated for C₂₃H₁₅NO₆: 402.38. found 402.36. LC/MS retention time: 2.70 minutes.

### Example 13: N-{2-[3-Cyano-4-(4-methoxyphenyl)-5-methylthiophen-2-yl]-1,3-dioxo-2,3-dihydro-1H-isoindole-5-carbonyl}-benzenesulfonamide

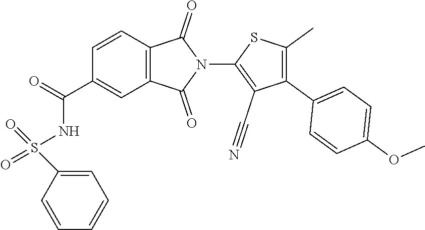 [SMILES: COc1ccc(-c2c(C)sc(N3C(=O)c4ccc(C(=O)NS(=O)(=O)c5ccccc5)cc4C3=O)c2C#N)cc1]

141. N-{2-[3-Cyano-4-(4-methoxyphenyl)-5-methylthiophen-2-yl]-1,3-dioxo-2,3-dihydro-1H-isoindole-5-carbonyl}-benzenesulfonamide was prepared as described in synthetic procedure K2 from crude N-(benzenesulfonyl)-1,3-dioxo-1,3-dihydro-2-benzofuran-5-carboxamide (Intermediate 11c) and 2-amino-4-(4-methoxyphenyl)-5-methylthiophene-3-carbonitrile (Intermediate 7). MS m/z: (M+H)⁺ calculated for C₂₈H₁₅N₃O₆S₂: 558.61. found 558.81. LC/MS retention time: 2.89 minutes.

### Example 14: N-{2-[3-Cyano-4-(4-methoxyphenyl)-5-methylthiophen-2-yl]-1,3-dioxo-2,3-dihydro-1H-isoindole-5-carbonyl}-methanesulfonamide

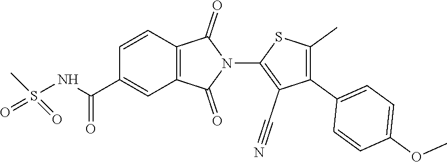 [SMILES: COc1ccc(-c2c(C)sc(N3C(=O)c4ccc(C(=O)NS(C)(=O)=O)cc4C3=O)c2C#N)cc1]

142. N-{2-[3-Cyano-4-(4-methoxyphenyl)-5-methylthiophen-2-yl]-1,3-dioxo-2,3-dihydro-1H-isoindole-5-carbonyl}-methanesulfonamide was prepared as described in synthetic procedure K2 from crude N-(methanesulfonyl)-1,3-dioxo-1,3-dihydro-2-benzofuran-5-carboxamide (Intermediate 11a) and 2-amino-4-(4-methoxyphenyl)-5-methylthiophene-3-carbonitrile (Intermediate 7); MS m/z: (M+H)⁺ calculated for C₂₃H₁₇N₃O₆S₂: 496.54. found 496.24. LC/MS retention time: 2.39 minutes.

### Example 15: Butane-1-sulfonic acid {2-[3-cyano-4-(4-methoxyphenyl)-5-methylthiophen-2-yl]-1,3-dioxo-2,3-dihydro-1H-isoindole-5-carbonyl}-amide

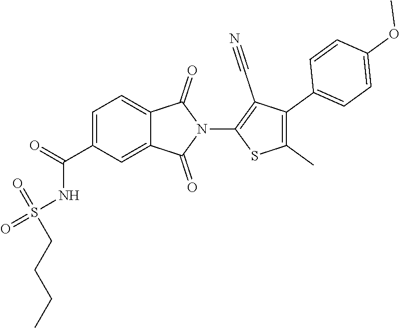 [SMILES: CCCCS(=O)(=O)NC(=O)c1ccc2c(c1)C(=O)N(c1sc(C)c(-c3ccc(OC)cc3)c1C#N)C2=O]

143. Butane-1-sulfonic acid {2-[3-cyano-4-(4-methoxyphenyl)-5-methylthiophen-2-yl]-1,3-dioxo-2,3-dihydro-1H-isoindole-5-carbonyl}-amide was prepared as described in synthetic procedure K2 from crude N-(butane-1-sulfonyl)-1,3-dioxo-1,3-dihydro-2-benzofuran-5-carboxamide (Intermediate 11b) and 2-amino-4-(4-methoxyphenyl)-5-methylthiophene-3-carbonitrile (Intermediate 7); MS m/z: (M+H)⁺ calculated for C₂₆H₂₃N₃O₆S₂: 538.62. found 538.97. LC/MS retention time: 2.28 minutes.

### Example 16: (2S)-2-{[2-(3-cyano-4-(4-methoxyphenyl)-5-methylthiophen-2-yl)-1,3-dioxo-2,3-dihydro-1H-isoindole-5-carbonyl]amino}-3-hydroxypropanoic acid

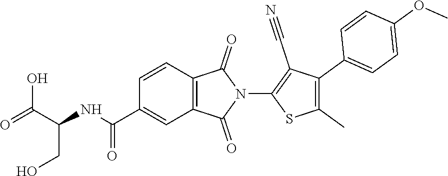 [SMILES: COc1ccc(-c2c(C)sc(N3C(=O)c4ccc(C(=O)N[C@@H](CO)C(=O)O)cc4C3=O)c2C#N)cc1]

144. (2S)-2-{[2-(3-cyano-4-(4-methoxyphenyl)-5-methylthiophen-2-yl)-1,3-dioxo-2,3-dihydro-1H-isoindole-5-carbonyl]amino}-3-hydroxypropanoic acid was prepared as described in synthetic procedure AA starting from 2-[3-cyano-4-(4-methoxyphenyl)-5-methylthiophen-2-yl]-1,3-dioxo-2,3-dihydro-1H-isoindole-5-carboxylic acid (see Example 6) and N-Fmoc-L-serine; MS m/z: (M+H)⁺ calculated for C₂₅H₁₉N₃O₇S: 506.51. found 505.91. LC/MS retention time: 2.51 minutes.

### Example 17: (2S)-2-({2-[3-Cyano-4-(4-methoxyphenyl)-5-methylthiophen-2-yl]-1,3-dioxo-2,3-dihydro-1H-isoindole-5-carbonyl}amino)-3-(1H-indol-3-yl)propionic acid

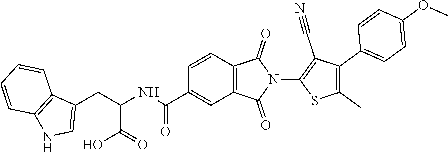 [SMILES: COc1ccc(-c2c(C)sc(N3C(=O)c4ccc(C(=O)NC(Cc5c[nH]c6ccccc56)C(=O)O)cc4C3=O)c2C#N)cc1]

145. (2S)-2-({2-[3-Cyano-4-(4-methoxyphenyl)-5-methylthiophen-2-yl]-1,3-dioxo-2,3-dihydro-1H-isoindole-5-carbonyl}amino)-3-(1H-indol-3-yl)propionic acid was prepared as described in synthetic procedure AA starting from 2-[3-cyano-4-(4-methoxyphenyl)-5-methylthiophen-2-yl]-1,3-dioxo-2,3-dihydro-1H-isoindole-5-carboxylic acid (see Example 6) and Fmoc-L-tryptophan. MS m/z: (M+H)⁺ calculated for C₃₃H₂₄N₄O₆S: 605.65. found 605.56. LC/MS retention time: 2.81 minutes.

### Example 18: (2S)-2-({2-[3-Cyano-4-(4-methoxyphenyl)-5-methylthiophen-2-yl]-1,3-dioxo-2,3-dihydro-1H-isoindole-5-carbonyl}-amino)-4-methyl-pentanoic acid

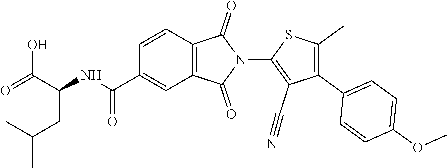 [SMILES: COc1ccc(-c2c(C)sc(N3C(=O)c4ccc(C(=O)N[C@@H](CC(C)C)C(=O)O)cc4C3=O)c2C#N)cc1]

146. (2S)-2-({2-[3-Cyano-4-(4-methoxyphenyl)-5-methylthiophen-2-yl]-1,3-dioxo-2,3-dihydro-1H-isoindole-5-carbonyl}-amino)-4-methyl-pentanoic acid was prepared as described in synthetic procedure AA starting from 2-[3-cyano-4-(4-methoxyphenyl)-5-methylthiophen-2-yl]-1,3-dioxo-2,3-dihydro-1H-isoindole-5-carboxylic acid (see Example 6) and Fmoc-L-leucine; MS m/z: (M+H)⁺ calculated for C₂₈H₂₅N₃O₆S: 532.59. found 532.02. LC/MS retention time: 2.85 minutes.

### Example 19: (2S)-2-({2-[3-Cyano-4-(4-methoxyphenyl)-5-methyl-thiophen-2-yl]-1,3-dioxo-2,3-dihydro-1H-isoindole-5-carbonyl}-amino)-3-methyl-butyric acid

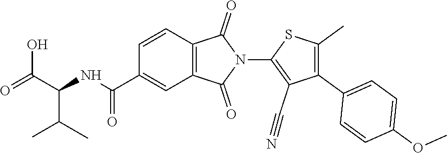 [SMILES: COc1ccc(-c2c(C)sc(N3C(=O)c4ccc(C(=O)N[C@H](C(=O)O)C(C)C)cc4C3=O)c2C#N)cc1]

(2S)-2-({2-[3-Cyano-4-(4-methoxyphenyl)-5-methyl-thiophen-2-yl]-1,3-dioxo-2,3-dihydro-1H-isoindole-5-carbonyl}-amino)-3-methyl-butyric acid was prepared as described in synthetic procedure AA starting from 2-[3-cyano-4-(4-methoxyphenyl)-5-methylthiophen-2-yl]-1,3-dioxo-2,3-dihydro-1H-isoindole-5-carboxylic acid (see Example 6) and Fmoc-L-valine; MS m/z: (M+H)⁺ calculated for C₂₇H₂₃N₃O₆S: 518.56. found 518.22. LC/MS retention time: 2.75 minutes.

### Example 20: (2S)-2-({2-[3-Cyano-4-(4-methoxyphenyl)-5-methyl-thiophen-2-yl]-1,3-dioxo-2,3-dihydro-1H-isoindole-5-carbonyl}-amino)-succinamic Acid

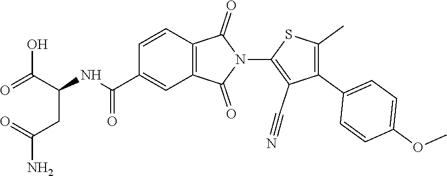 [SMILES: COc1ccc(-c2c(C)sc(N3C(=O)c4ccc(C(=O)N[C@@H](CC(N)=O)C(=O)O)cc4C3=O)c2C#N)cc1]

147. (2S)-2-({2-[3-Cyano-4-(4-methoxyphenyl)-5-methyl-thiophen-2-yl]-1,3-dioxo-2,3-dihydro-1H-isoindole-5-carbonyl}-amino)-succinamic acid was prepared as described in synthetic procedure AA starting from 2-[3-cyano-4-(4-methoxyphenyl)-5-methylthiophen-2-yl]-1,3-dioxo-2,3-dihydro-1H-isoindole-5-carboxylic acid (see Example 6) and N-alpha-Fmoc-L-asparagine; MS m/z: (M+H)⁺ calculated for C₂₆H₂₀N₄O₇S: 533.54. found 533.22. LC/MS retention time: 2.42 minutes.

### Example 21: (2S)-4-Carbamoyl-2-({2-[3-cyano-4-(4-methoxyphenyl)-5-methyl-thiophen-2-yl]-1,3-dioxo-2,3-dihydro-1H-isoindole-5-carbonyl}-amino)-butyric Acid

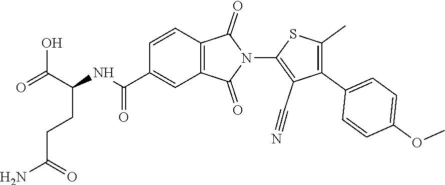 [SMILES: COc1ccc(-c2c(C)sc(N3C(=O)c4ccc(C(=O)N[C@@H](CCC(N)=O)C(=O)O)cc4C3=O)c2C#N)cc1]

148. (2S)-4-Carbamoyl-2-({2-[3-cyano-4-(4-methoxyphenyl)-5-methyl-thiophen-2-yl]-1,3-dioxo-2,3-dihydro-1H-isoindole-5-carbonyl}-amino)-butyric acid was prepared as described in synthetic procedure AA starting from 2-[3-cyano-4-(4-methoxyphenyl)-5-methylthiophen-2-yl]-1,3-dioxo-2,3-dihydro-1H-isoindole-5-carboxylic acid (see Example 6) and Fmoc-L-glutamine; MS m/z: (M+H)⁺ calculated for C₂₇H₂₂N₄O₇S: 547.56. found 547.03. LC/MS retention time: 2.43 minutes.

### Example 22: 4-(5-Benzenesulfonylaminocarbonyl-1,3-dioxo-1,3-dihydroisoindol-2-yl)biphenyl-3-carboxylic Acid

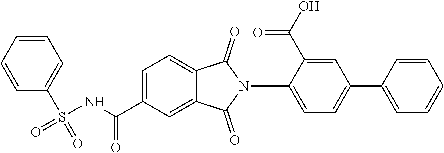 [SMILES: O=C(NS(=O)(=O)c1ccccc1)c1ccc2c(c1)C(=O)N(c1ccc(-c3ccccc3)cc1C(=O)O)C2=O]

149. 4-(5-Benzenesulfonylaminocarbonyl-1,3-dioxo-1,3-dihydroisoindol-2-yl)biphenyl-3-carboxylic acid was prepared as described in synthetic procedure K2 from crude N-(benzenesulfonyl)-1,3-dioxo-1,3-dihydro-2-benzofuran-5-carboxamide (Intermediate 11c) and 4-amino[1,1′-biphenyl]-3-carboxylic acid (Intermediate 13); MS m/z: (M+H)⁺ calculated for C₂₈H₁₈N₂O₇S: 527.53. found 527.82. LC/MS retention time: 2.29 minutes.

### Example 23: 4-(5-Methanesulfonylaminocarbonyl-1,3-dioxo-1,3-dihydroisoindol-2-yl)biphenyl-3-carboxylic Acid

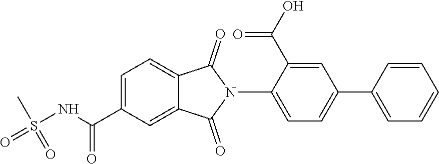 [SMILES: CS(=O)(=O)NC(=O)c1ccc2c(c1)C(=O)N(c1ccc(-c3ccccc3)cc1C(=O)O)C2=O]

150. 4-(5-Methanesulfonylaminocarbonyl-1,3-dioxo-1,3-dihydroisoindol-2-yl)biphenyl-3-carboxylic acid was prepared as described in synthetic procedure K2 from crude N-(methanesulfonyl)-1,3-dioxo-1,3-dihydro-2-benzofuran-5-carboxamide (Intermediate 11a) and 4-amino[1,1′-biphenyl]-3-carboxylic acid (Intermediate 13); MS m/z: (M+H)⁺ calculated for C₂₃H₁₆N₂O₇S: 465.46. found 465.82. LC/MS retention time: 2.19 minutes.

### Example 24: 4-[5-(Butane-1-sulfonylaminocarbonyl)-1,3-dioxo-1,3-dihydroisoindol-2-yl]biphenyl-3-carboxylic Acid

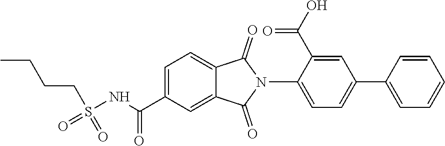 [SMILES: CCCCS(=O)(=O)NC(=O)c1ccc2c(c1)C(=O)N(c1ccc(-c3ccccc3)cc1C(=O)O)C2=O]

151. 44-[5-(Butane-1-sulfonylaminocarbonyl)-1,3-dioxo-1,3-dihydroisoindol-2-yl]biphenyl-3-carboxylic acid was prepared as described in synthetic procedure K2 from crude N-(butane-1-sulfonyl)-1,3-dioxo-1,3-dihydro-2-benzofuran-5-carboxamide (Intermediate 11b) and 4-amino[1,1′-biphenyl]-3-carboxylic acid (Intermediate 13); MS m/z: (M+H)⁺ calculated for C₂₆H₂₂N₂O₇S: 507.54. found 507.99. LC/MS retention time: 2.41 minutes.

### Example 25: 3-(5-Methanesulfonylaminocarbonyl-1,3-dioxo-1,3-dihydroisoindol-2-yl)biphenyl-4-carboxylic Acid

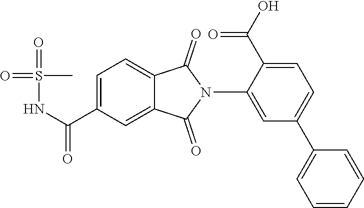 [SMILES: CS(=O)(=O)NC(=O)c1ccc2c(c1)C(=O)N(c1cc(-c3ccccc3)ccc1C(=O)O)C2=O]

152. 3-(5-Methanesulfonylaminocarbonyl-1,3-dioxo-1,3-dihydroisoindol-2-yl)biphenyl-4-carboxylic acid was prepared as described in synthetic procedure K2 from crude N-(methanesulfonyl)-1,3-dioxo-1,3-dihydro-2-benzofuran-5-carboxamide (Intermediate 11a) and 3-amino[1,1′-biphenyl]-4-carboxylic acid; MS m/z: (M+H)⁺ calculated for C₂₃H₁₆N₂O₇S: 465.46. found 465.78. LC/MS retention time: 2.27 minutes.

### Example 26: 3-[5-(Butane-1-sulfonylaminocarbonyl)-1,3-dioxo-1,3-dihydroisoindol-2-yl]biphenyl-4-carboxylic Acid

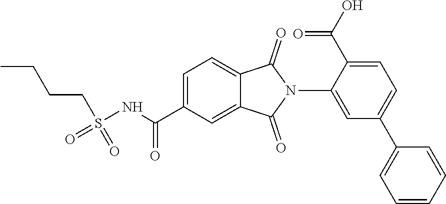 [SMILES: CCCCS(=O)(=O)NC(=O)c1ccc2c(c1)C(=O)N(c1cc(-c3ccccc3)ccc1C(=O)O)C2=O]

153. 3-[5-(Butane-1-sulfonylaminocarbonyl)-1,3-dioxo-1,3-dihydroisoindol-2-yl]biphenyl-4-carboxylic acid was prepared as described in synthetic procedure K2 from crude N-(butane-1-sulfonyl)-1,3-dioxo-1,3-dihydro-2-benzofuran-5-carboxamide (Intermediate 11b) and 3-amino[1,1′-biphenyl]-4-carboxylic acid; MS m/z: (M+H)⁺ calculated for C₂₆H₂₂N₂O₇S: 507.54. found 507.84. LC/MS retention time: 2.43 minutes.

### Example 27: 4-(5-Carbamoyl-1,3-dioxo-1,3-dihydroisoindol-2-yl)biphenyl-3-carboxylic Acid

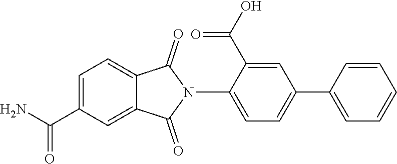 [SMILES: NC(=O)c1ccc2c(c1)C(=O)N(c1ccc(-c3ccccc3)cc1C(=O)O)C2=O]

154. 4-(5-Carbamoyl-1,3-dioxo-1,3-dihydroisoindol-2-yl)biphenyl-3-carboxylic acid was prepared as described in synthetic procedure K1 from 1,3-dioxo-1,3-dihydro-2-benzofuran-5-carboxamide and 4-amino[1,1′-biphenyl]-3-carboxylic acid (Intermediate 13); MS m/z: (M+H)⁺ calculated for C₂₂H₁₄N₂O₅: 387.37. found 387.56. LC/MS retention time: 2.21 minutes.

### Example 28: 3-(5-Carbamoyl-1,3-dioxo-1,3-dihydroisoindol-2-yl)biphenyl-4-carboxylic Acid

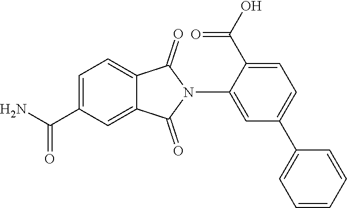 [SMILES: NC(=O)c1ccc2c(c1)C(=O)N(c1cc(-c3ccccc3)ccc1C(=O)O)C2=O]

155. 3-(5-Carbamoyl-1,3-dioxo-1,3-dihydroisoindol-2-yl)biphenyl-4-carboxylic acid was prepared as described in synthetic procedure K1 from 1,3-dioxo-1,3-dihydro-2-benzofuran-5-carboxamide and 3-amino[1,1′-biphenyl]-4-carboxylic acid; MS m/z: (M+H)⁺ calculated for C₂₂H₁₄N₂O₅: 387.37. found 387.67.

LC/MS retention time: 2.25 minutes.

### Example 29: 4-[5-(1-Benzyl-1H-[1,2,3]triazol-4-yl)-1,3-dioxo-1,3-dihydroisoindol-2-yl]biphenyl-3-carboxylic Acid

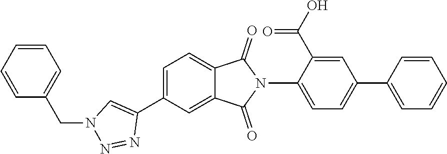 [SMILES: O=C(O)c1cc(-c2ccccc2)ccc1N1C(=O)c2ccc(-c3cn(Cc4ccccc4)nn3)cc2C1=O]

156. 4-[5-(1-Benzyl-1H-[1,2,3]triazol-4-yl)-1,3-dioxo-1,3-dihydroisoindol-2-yl]biphenyl-3-carboxylic acid was prepared as described in synthetic procedures J1 followed by synthetic procedure L from 4-ethynylbenzene-1,2-dicarboxylic acid (Intermediate 4a), benzyl azide solution and 4-amino[1,1′-biphenyl]-3-carboxylic acid (Intermediate 13); MS m/z: (M+H)⁺ calculated for C₃₀H₂₀N₄O₄: 501.52. found 501.41. LC/MS retention time: 2.62 minutes.

### Example 30: 4-{5-[1-(2,2-Dimethylpropionyloxymethyl)-1H-[1,2,3]triazol-4-yl]-1,3-dioxo-1,3-dihydroisoindol-2-yl}biphenyl-3-carboxylic Acid

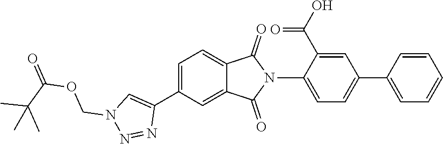 [SMILES: CC(C)(C)C(=O)OCn1cc(-c2ccc3c(c2)C(=O)N(c2ccc(-c4ccccc4)cc2C(=O)O)C3=O)nn1]

157. 4-{5-[1-(2,2-Dimethylpropionyloxymethyl)-1H-[1,2,3]triazol-4-yl]-1,3-dioxo-1,3-dihydroisoindol-2-yl}biphenyl-3-carboxylic acid was prepared as described in synthetic procedures J1 followed by synthetic procedure L from 4-ethynylbenzene-1,2-dicarboxylic acid (Intermediate 4a), azidomethyl pivalate and 4-amino[1,1′-biphenyl]-3-carboxylic acid (Intermediate 13); MS m/z: (M+H)⁺ calculated for C₂₉H₂₄N₄O₆: 525.54. found 525.42. LC/MS retention time: 2.71 minutes.

### Example 31: 4-[1,3-Dioxo-5-(1H-[1,2,3]triazol-4-yl)-1,3-dihydroisoindol-2-yl]biphenyl-3-carboxylic Acid

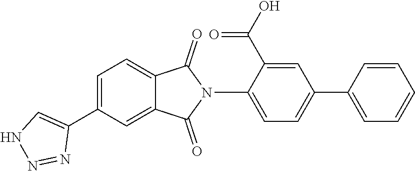 [SMILES: O=C(O)c1cc(-c2ccccc2)ccc1N1C(=O)c2ccc(-c3c[nH]nn3)cc2C1=O]

158. 4-[1,3-Dioxo-5-(1H-[1,2,3]triazol-4-yl)-1,3-dihydroisoindol-2-yl]biphenyl-3-carboxylic acid was prepared as described in synthetic Procedure L from 4-(1H-1,2,3-triazol-4-yl)phthalic acid (Intermediate 5) and 4-amino[1,1′-biphenyl]-3-carboxylic acid (Intermediate 13); MS m/z: (M+H)⁺ calculated for C₂₃H₁₄N₄O₄: 411.39. found 411.36. LC/MS retention time: 2.13 minutes.

### Example 32: 2-[1,3-Dioxo-5-(1H-[1,2,3]triazol-4-yl)-1,3-dihydroisoindol-2-yl]-4-(4-methoxyphenyl)-5-methylthiophene-3-carbonitrile

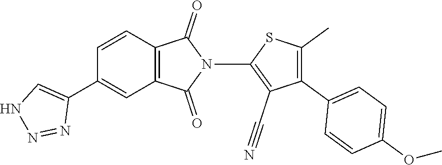 [SMILES: COc1ccc(-c2c(C)sc(N3C(=O)c4ccc(-c5c[nH]nn5)cc4C3=O)c2C#N)cc1]

159. 2-[1,3-Dioxo-5-(1H-[1,2,3]triazol-4-yl)-1,3-dihydroisoindol-2-yl]-4-(4-methoxyphenyl)-5-methylthiophene-3-carbonitrile was prepared as described in synthetic Procedure L from 4-(1H-1,2,3-triazol-4-yl)phthalic acid (Intermediate 5) and 2-amino-4-(4-methoxyphenyl)-5-methylthiophene-3-carbonitrile (Intermediate 7); MS m/z: (M+H)⁺ calculated for C₂₃H₁₄N₄O₄: 442.47. found 442.28. LC/MS retention time: 2.47 minutes.

### Example 33: 4-[1,3-Dioxo-5-(1H-[1,2,3]triazol-4-yl)-1,3-dihydroisoindol-2-yl]biphenyl-3-carboxylic acid methyl ester

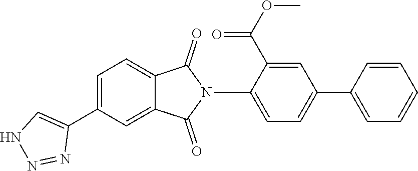 [SMILES: COC(=O)c1cc(-c2ccccc2)ccc1N1C(=O)c2ccc(-c3c[nH]nn3)cc2C1=O]

160. 4-[1,3-Dioxo-5-(1H-[1,2,3]triazol-4-yl)-1,3-dihydroisoindol-2-yl]biphenyl-3-carboxylic acid methyl ester was prepared as described in synthetic procedure L from 4-(1H-1,2,3-triazol-4-yl)phthalic acid (Intermediate 5) and methyl 4-amino[1,1′-biphenyl]-3-carboxylate (Intermediate 12); MS m/z: (M+H)⁺ calculated for C₂₄H₁₆N₄O₄: 425.42. found 425.17. LC/MS retention time: 2.49 minutes.

### Example 34: 3-[1,3-Dioxo-5-(1H-[1,2,3]triazol-4-yl)-1,3-dihydroisoindol-2-yl]biphenyl-4-carboxylic acid methyl ester

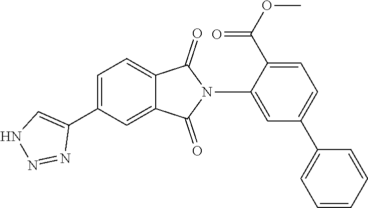 [SMILES: COC(=O)c1ccc(-c2ccccc2)cc1N1C(=O)c2ccc(-c3c[nH]nn3)cc2C1=O]

161. 3-[1,3-Dioxo-5-(1H-[1,2,3]triazol-4-yl)-1,3-dihydroisoindol-2-yl]biphenyl-4-carboxylic acid methyl ester was prepared as described in synthetic procedure L from 4-(1H-1,2,3-triazol-4-yl)phthalic acid (Intermediate 5) and methyl 3-amino[1,1′-biphenyl]-4-carboxylate; MS m/z: (M+H)⁺ calculated for C₂₄H₁₆N₄O₄: 425.42. found 425.18. LC/MS retention time: 2.42 minutes.

### Example 35: 2-(3-Methoxycarbonylbiphenyl-4-yl)-1,3-dioxo-2,3-dihydro-1H-isoindole-5-carboxylic acid butyl ester

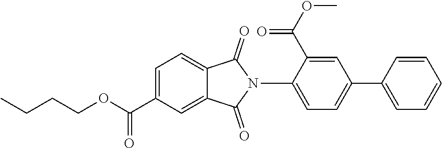 [SMILES: CCCCOC(=O)c1ccc2c(c1)C(=O)N(c1ccc(-c3ccccc3)cc1C(=O)OC)C2=O]

162. 2-(3-Methoxycarbonylbiphenyl-4-yl)-1,3-dioxo-2,3-dihydro-1H-isoindole-5-carboxylic acid butyl ester was prepared as described in synthetic procedure AD from 2-(3-methoxycarbonylbiphenyl-4-yl)-1,3-dioxo-2,3-dihydro-1H-isoindole-5-carboxylic acid (see Example 12) and n-butanol; MS m/z: (M+H)⁺ calculated for C₂₇H₂₃NO₆: 458.49. found 458.59. LC/MS retention time: 3.16 minutes.

### Example 36: 2-(4-Methoxycarbonylbiphenyl-3-yl)-1,3-dioxo-2,3-dihydro-1H-isoindole-5-carboxylic acid butyl ester

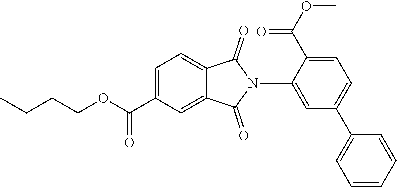 [SMILES: CCCCOC(=O)c1ccc2c(c1)C(=O)N(c1cc(-c3ccccc3)ccc1C(=O)OC)C2=O]

163. 2-(4-Methoxycarbonylbiphenyl-3-yl)-1,3-dioxo-2,3-dihydro-1H-isoindole-5-carboxylic acid butyl ester was prepared as described in synthetic procedure AD from 2-(4-methoxycarbonylbiphenyl-3-yl)-1,3-dioxo-2,3-dihydro-1H-isoindole-5-carboxylic acid (see Example 37) and n-butanol; MS m/z: (M+H)⁺ calculated for C₂₇H₂₃NO₆: 458.49. found 458.63. LC/MS retention time: 3.17 minutes.

### Example 37: 2-(4-Methoxycarbonylbiphenyl-3-yl)-1,3-dioxo-2,3-dihydro-1H-isoindole-5-carboxylic Acid

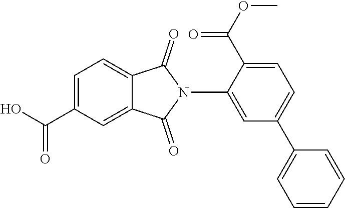 [SMILES: COC(=O)c1ccc(-c2ccccc2)cc1N1C(=O)c2ccc(C(=O)O)cc2C1=O]

164. 2-(4-Methoxycarbonylbiphenyl-3-yl)-1,3-dioxo-2,3-dihydro-1H-isoindole-5-carboxylic acid was prepared as described in synthetic procedure K from trimellitic anhydride and methyl 3-amino[1,1′-biphenyl]-4-carboxylate; MS m/z: (M+H)⁺ calculated for C₂₃H₁₅NO₆: 402.38. found 402.36. LC/MS retention time: 2.41 minutes.

### Example 38: 4-[1,3-Dioxo-5-(1H-[1,2,3]triazol-4-yl)-1,3-dihydroisoindol-2-yl]-6-methoxybiphenyl-3-carboxylic acid methyl ester

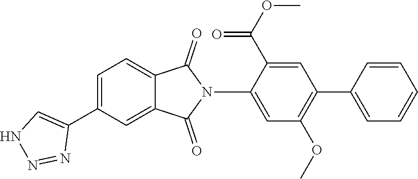 [SMILES: COC(=O)c1cc(-c2ccccc2)c(OC)cc1N1C(=O)c2ccc(-c3c[nH]nn3)cc2C1=O]

165. 4-[1,3-Dioxo-5-(1H-[1,2,3]triazol-4-yl)-1,3-dihydroisoindol-2-yl]-6-methoxybiphenyl-3-carboxylic acid methyl ester was prepared as described in synthetic procedure L starting from 4-(1H-1,2,3-triazol-4-yl)phthalic acid (Intermediate 5) and methyl 4-amino-6-methoxy[1,1′-biphenyl]-3-carboxylate (Intermediate 10); MS m/z: (M+H)⁺ calculated for C₂₅H₁N₄O₅: 455.45. found 455.48. LC/MS retention time: 2.43 minutes.

### Example 39: 2-(2-Methoxy-5-methoxycarbonylbiphenyl-4-yl)-1,3-dioxo-2,3-dihydro-1H-isoindole-5-carboxylic acid butyl ester

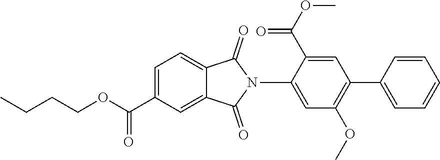 [SMILES: CCCCOC(=O)c1ccc2c(c1)C(=O)N(c1cc(OC)c(-c3ccccc3)cc1C(=O)OC)C2=O]

166. 2-(2-Methoxy-5-methoxycarbonylbiphenyl-4-yl)-1,3-dioxo-2,3-dihydro-1H-isoindole-5-carboxylic acid butyl ester was prepared as described in synthetic procedure AD from 2-(6-methoxy-3-methoxycarbonylbiphenyl-4-yl)-1,3-dioxo-2,3-dihydro-1H-isoindole-5-carboxylic acid (see Example 42) and n-butanol; MS m/z: (M+H)⁺ calculated for C₂₈H₂₅NO₇: 488.51. found 488.50. LC/MS retention time: 3.15 minutes.

### Example 40: 4-(5-Methanesulfonylaminocarbonyl-1,3-dioxo-1,3-dihydroisoindol-2-yl)biphenyl-3-carboxylic acid methyl ester

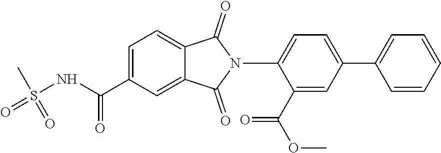 [SMILES: COC(=O)c1cc(-c2ccccc2)ccc1N1C(=O)c2ccc(C(=O)NS(C)(=O)=O)cc2C1=O]

167. 4-(5-Methanesulfonylaminocarbonyl-1,3-dioxo-1,3-dihydroisoindol-2-yl)biphenyl-3-carboxylic acid methyl ester was prepared as described in K2 from crude N-(methanesulfonyl)-1,3-dioxo-1,3-dihydro-2-benzofuran-5-carboxamide (Intermediate 11a) and methyl 4-amino[1,1′-biphenyl]-3-carboxylate (Intermediate 12); MS m/z: (M+H)⁺ calculated for C₂₄H₁₈N₂O₇S: 478.48. found 478.61. LC/MS retention time: 2.53 minutes.

### Example 41: 3-(5-Methanesulfonylaminocarbonyl-1,3-dioxo-1,3-dihydroisoindol-2-yl)biphenyl-4-carboxylic acid methyl ester

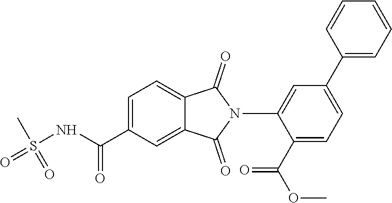 [SMILES: COC(=O)c1ccc(-c2ccccc2)cc1N1C(=O)c2ccc(C(=O)NS(C)(=O)=O)cc2C1=O]

168. 3-(5-Methanesulfonylaminocarbonyl-1,3-dioxo-1,3-dihydroisoindol-2-yl)biphenyl-4-carboxylic acid methyl ester was prepared as described in K2 from crude N-(methanesulfonyl)-1,3-dioxo-1,3-dihydro-2-benzofuran-5-carboxamide (Intermediate 11a) and methyl 3-amino[1,1′-biphenyl]-4-carboxylate; MS m/z: (M+H)⁺ calculated for C₂₄H₁₈N₂O₇S: 478.48. found 478.43. LC/MS retention time: 2.51 minutes.

### Example 42: 2-(6-Methoxy-3-methoxycarbonylbiphenyl-4-yl)-1,3-dioxo-2,3-dihydro-1H-isoindole-5-carboxylic Acid

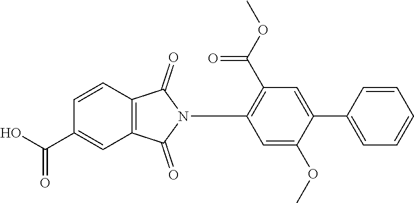 [SMILES: COC(=O)c1cc(-c2ccccc2)c(OC)cc1N1C(=O)c2ccc(C(=O)O)cc2C1=O]

169. 2-(6-Methoxy-3-methoxycarbonylbiphenyl-4-yl)-1,3-dioxo-2,3-dihydro-1H-isoindole-5-carboxylic acid was prepared as described in synthetic procedure K from trimellitic anhydride and methyl 4-amino-6-methoxy[1,1′-biphenyl]-3-carboxylate (Intermediate 10); MS m/z: (M+H)⁺ calculated for C₂₄H₁₇NO₇: 432.41. found 432.37. LC/MS retention time: 2.50 minutes.

### Example 43: 1,3-Dioxo-2-[4-(1H-tetrazol-5-yl)biphenyl-3-yl]-2,3-dihydro-1H-isoindole-5-carboxylic Acid

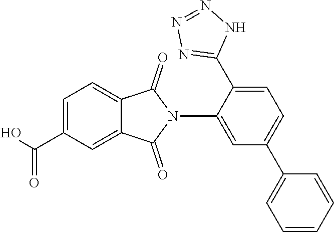 [SMILES: O=C(O)c1ccc2c(c1)C(=O)N(c1cc(-c3ccccc3)ccc1-c1nnn[nH]1)C2=O]

170. 1,3-Dioxo-2-[4-(1H-tetrazol-5-yl)biphenyl-3-yl]-2,3-dihydro-1H-isoindole-5-carboxylic acid was prepared as described in synthetic procedure K from trimellitic anhydride and crude 4-(1H-tetrazol-5-yl)[1,1′-biphenyl]-3-amine (Intermediate 19); MS m/z: (M+H)⁺ calculated for C₂₂H₁₃N₅O₄: 412.38. found 412.26. LC/MS retention time: 2.03 minutes.

### Example 44: 3-[1,3-Dioxo-5-(1H-tetrazol-5-yl)-1,3-dihydroisoindol-2-yl]biphenyl-4-carboxylic acid methyl ester

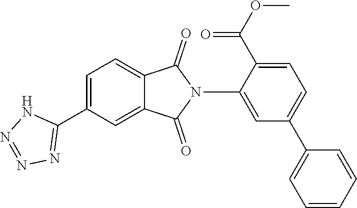 [SMILES: COC(=O)c1ccc(-c2ccccc2)cc1N1C(=O)c2ccc(-c3nnn[nH]3)cc2C1=O]

171. 3-[1,3-Dioxo-5-(1H-tetrazol-5-yl)-1,3-dihydroisoindol-2-yl]biphenyl-4-carboxylic acid methyl ester was prepared as described in synthetic procedure M from 4-cyano-1,2-benzenedicarboxylic acid, methyl 3-amino[1,1′-biphenyl]-4-carboxylate and sodium azide; MS m/z: (M+H)⁺ calculated for C₂₃H₁₅N₅O₄: 426.41. found 426.61. LC/MS retention time: 2.36 minutes.

### Example 45: 2-(4-Carboxy-4′-fluorobiphenyl-3-yl)-1,3-dioxo-2,3-dihydro-1H-isoindole-5-carboxylic Acid

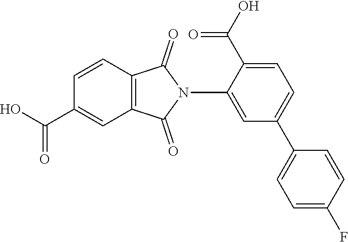 [SMILES: O=C(O)c1ccc2c(c1)C(=O)N(c1cc(-c3ccc(F)cc3)ccc1C(=O)O)C2=O]

172. 2-(4-Carboxy-4′-fluorobiphenyl-3-yl)-1,3-dioxo-2,3-dihydro-1H-isoindole-5-carboxylic acid was prepared as described in synthetic procedure K from trimellitic anhydride and 3-amino-4′-fluorobiphenyl-4-carboxylic acid (was prepared from methyl 3-amino-4′-fluoro[1,1′-biphenyl]-4-carboxylate (Intermediate 14) as described in synthetic procedure B); MS m/z: (M+H)⁺ calculated for C₂₂H₁₂FNO₆: 406.34. found 406.26. LC/MS retention time: 2.29 minutes.

### Example 46: 2-(4-Carboxy-3′-fluorobiphenyl-3-yl)-1,3-dioxo-2,3-dihydro-1H-isoindole-5-carboxylic Acid

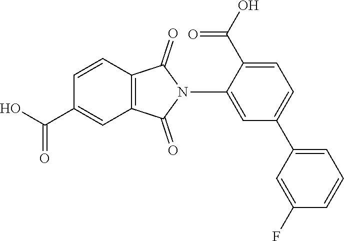 [SMILES: O=C(O)c1ccc2c(c1)C(=O)N(c1cc(-c3cccc(F)c3)ccc1C(=O)O)C2=O]

173. 2-(4-Carboxy-3′-fluorobiphenyl-3-yl)-1,3-dioxo-2,3-dihydro-1H-isoindole-5-carboxylic acid was prepared as described in synthetic procedure K from trimellitic anhydride and 3-amino-3′-fluoro[1,1′-biphenyl]-4-carboxylic acid (Intermediate 15); MS m/z: (M+H)⁺ calculated for C₂₂H₁₂FNO₆: 406.34. found 405.96. LC/MS retention time: 2.29 minutes.

### Example 47: 2-[5-Methoxy-2-(1H-tetrazol-5-yl)phenyl]-1,3-dioxo-2,3-dihydro-1H-isoindole-5-carboxylic acid

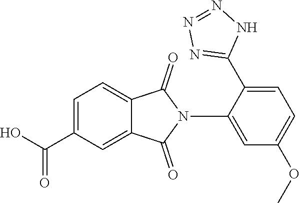 [SMILES: COc1ccc(-c2nnn[nH]2)c(N2C(=O)c3ccc(C(=O)O)cc3C2=O)c1]

174. 2-[5-Methoxy-2-(1H-tetrazol-5-yl)phenyl]-1,3-dioxo-2,3-dihydro-1H-isoindole-5-carboxylic acid was prepared as described in synthetic procedure K from trimellitic anhydride and 5-methoxy-2-(1H-tetrazol-5-yl)-benzenamine (was prepared from 2-amino-4-methoxybenzonitrile as described in J. Heterocycl. Chem., 1977(14), 561-564); MS m/z: (M+H)⁺ calculated for C₁₇H₁₁N₅O₅: 366.31. found 366.04. LC/MS retention time: 1.59 minutes.

### Example 48: 2-(2-Carboxy-5-thiophen-2-yl-phenyl)-1,3-dioxo-2,3-dihydro-1H-isoindole-5-carboxylic Acid

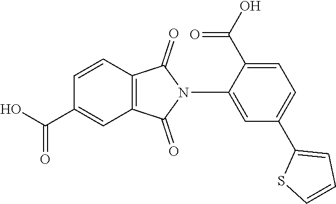 [SMILES: O=C(O)c1ccc2c(c1)C(=O)N(c1cc(-c3cccs3)ccc1C(=O)O)C2=O]

175. 2-(2-Carboxy-5-thiophen-2-yl-phenyl)-1,3-dioxo-2,3-dihydro-1H-isoindole-5-carboxylic acid was prepared as described in synthetic procedure K from trimellitic anhydride and 2-amino-4-(thiophen-2-yl)benzoic acid; MS m/z: (M+H)⁺ calculated for C₂₀H₁₁N₅O₆S: 394.38. found 393.95. LC/MS retention time: 2.11 minutes.

### Example 49: 2-[1,3-Dioxo-5-(1H-[1,2,3]triazol-4-yl)-1,3-dihydroisoindol-2-yl]-4-pyridin-3-yl-benzoic acid

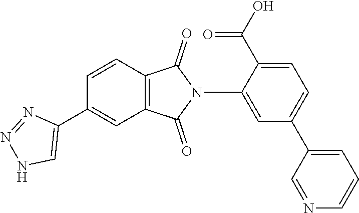 [SMILES: O=C(O)c1ccc(-c2cccnc2)cc1N1C(=O)c2ccc(-c3c[nH]nn3)cc2C1=O]

176. 2-[1,3-Dioxo-5-(1H-[1,2,3]triazol-4-yl)-1,3-dihydroisoindol-2-yl]-4-pyridin-3-yl-benzoic acid was prepared as described in synthetic procedure L starting from 4-(1H-1,2,3-triazol-4-yl)phthalic acid (Intermediate 5) and 2-amino-4-(pyridin-3-yl)benzoic acid (Intermediate 17); MS m/z: (M+H)⁺ calculated for C₂₂H₁₃N₅O₄: 412.38. found 412.26. LC/MS retention time: 1.19 minutes.

### Example 50: 3-[1,3-Dioxo-5-(1H-[1,2,3]triazol-4-yl)-1,3-dihydroisoindol-2-yl]-3′-fluorobiphenyl-4-carboxylic Acid

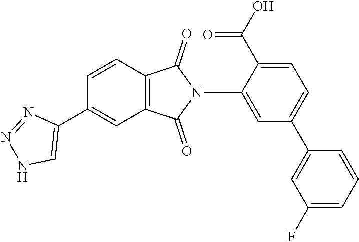 [SMILES: O=C(O)c1ccc(-c2cccc(F)c2)cc1N1C(=O)c2ccc(-c3c[nH]nn3)cc2C1=O]

177. 3-[1,3-Dioxo-5-(1H-[1,2,3]triazol-4-yl)-1,3-dihydroisoindol-2-yl]-3′-fluorobiphenyl-4-carboxylic acid was prepared as described in synthetic procedure L from 4-(1H-1,2,3-triazol-4-yl)phthalic acid (Intermediate 5) and 3-amino-3′-fluoro[1,1′-biphenyl]-4-carboxylic acid (Intermediate 15); MS m/z: (M+H)⁺ calculated for C₂₃H₁₃FN₄O₄: 429.38. found 429.37. LC/MS retention time: 2.35 minutes.

### Example 51: 3-[1,3-Dioxo-5-(1H-[1,2,3]triazol-4-yl)-1,3-dihydroisoindol-2-yl]-2′,4′-difluorobiphenyl-4-carboxylic Acid

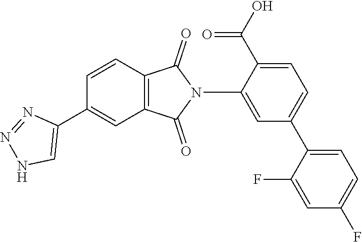 [SMILES: O=C(O)c1ccc(-c2ccc(F)cc2F)cc1N1C(=O)c2ccc(-c3c[nH]nn3)cc2C1=O]

178. 3-[1,3-Dioxo-5-(1H-[1,2,3]triazol-4-yl)-1,3-dihydroisoindol-2-yl]-2′,4′-difluorobiphenyl-4-carboxylic acid was prepared as described in synthetic procedure L from 4-(1H-1,2,3-triazol-4-yl)phthalic acid (Intermediate 5) and 3-amino-2′,4′-difluoro[1,1′-biphenyl]-4-carboxylic acid (Intermediate 16); MS m/z: (M+H)⁺ calculated for C₂₃H₁₂F₂N₄O₄: 447.37. found 447.38. LC/MS retention time: 2.29 minutes.

### Example 52: 2-(2-Carboxy-5-pyridin-3-yl-phenyl)-1,3-dioxo-2,3-dihydro-1H-isoindole-5-carboxylic Acid

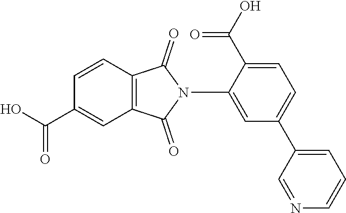 [SMILES: O=C(O)c1ccc2c(c1)C(=O)N(c1cc(-c3cccnc3)ccc1C(=O)O)C2=O]

179. 2-(2-Carboxy-5-pyridin-3-yl-phenyl)-1,3-dioxo-2,3-dihydro-1H-isoindole-5-carboxylic acid was prepared as described in synthetic procedure K from trimellitic anhydride and 2-amino-4-(pyridin-3-yl)benzoic acid (Intermediate 17); MS m/z: (M+H)⁺ calculated for C₂₁H₁₂N₂O₆: 389.34. found 389.54. LC/MS retention time: 2.09 minutes.

### Example 53: 2-(4-Carboxy-2′,4′-difluorobiphenyl-3-yl)-1,3-dioxo-2,3-dihydro-1H-isoindole-5-carboxylic acid

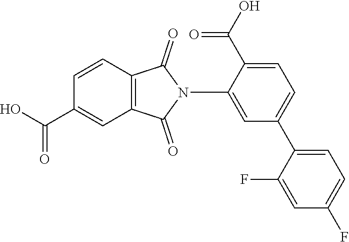 [SMILES: O=C(O)c1ccc2c(c1)C(=O)N(c1cc(-c3ccc(F)cc3F)ccc1C(=O)O)C2=O]

180. 2-(4-Carboxy-2′,4′-difluorobiphenyl-3-yl)-1,3-dioxo-2,3-dihydro-1H-isoindole-5-carboxylic acid was prepared as described in synthetic procedure K starting from trimellitic anhydride and 3-amino-2′,4′-difluoro[1,1′-biphenyl]-4-carboxylic acid (Intermediate 16); MS m/z: (M+H)⁺ calculated for C₂₂H₁₁F₂NO₆: 424.33. found 424.27. LC/MS retention time: 2.18 minutes.

### Example 54: 2-[4-(1H-Tetrazol-5-yl)biphenyl-3-yl]-5-(1H-[1,2,3]triazol-4-yl)-isoindole-1,3-dione

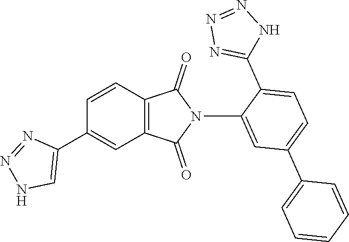 [SMILES: O=C1c2ccc(-c3c[nH]nn3)cc2C(=O)N1c1cc(-c2ccccc2)ccc1-c1nnn[nH]1]

181. 2-[4-(1H-Tetrazol-5-yl)biphenyl-3-yl]-5-(1H-[1,2,3]triazol-4-yl)-isoindole-1,3-dione was prepared as described in synthetic procedure L from 4-(1H-1,2,3-triazol-4-yl)phthalic acid (Intermediate 5) and crude 4-(1H-tetrazol-5-yl)[1,1′-biphenyl]-3-amine (Intermediate 19); MS m/z: (M+H)⁺ calculated for C₂₃H₁₄N₈O₂: 435.42. found 435.37. LC/MS retention time: 2.17 minutes.

### Example 55: 2-(4-Carboxy-4′-methoxybiphenyl-3-yl)-1,3-dioxo-2,3-dihydro-1H-isoindole-5-carboxylic Acid

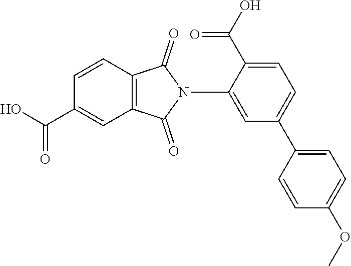 [SMILES: COc1ccc(-c2ccc(C(=O)O)c(N3C(=O)c4ccc(C(=O)O)cc4C3=O)c2)cc1]

182. 2-(4-Carboxy-4′-methoxybiphenyl-3-yl)-1,3-dioxo-2,3-dihydro-1H-isoindole-5-carboxylic acid was prepared as described in synthetic procedure K from trimellitic anhydride and 3-amino-4′-methoxy[1,1′-biphenyl]-4-carboxylic acid; MS m/z: (M+H)⁺ calculated for C₂₃H₁₅NO₇: 418.38. found 418.26. LC/MS retention time: 2.29 minutes.

### Example 56: 2-[1,3-Dioxo-5-(1H-[1,2,3]triazol-4-yl)-1,3-dihydroisoindol-2-yl]-4-thiophen-2-yl-benzoic Acid

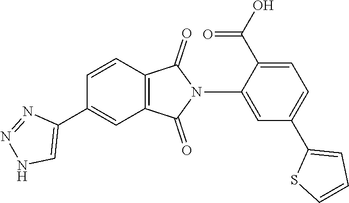 [SMILES: O=C(O)c1ccc(-c2cccs2)cc1N1C(=O)c2ccc(-c3c[nH]nn3)cc2C1=O]

183. 2-[1,3-Dioxo-5-(1H-[1,2,3]triazol-4-yl)-1,3-dihydroisoindol-2-yl]-4-thiophen-2-yl-benzoic acid was prepared as described in synthetic procedure L from 4-(1H-1,2,3-triazol-4-yl)phthalic acid (Intermediate 5) and 2-amino-4-(thiophen-2-yl)benzoic acid; MS m/z: (M+H)⁺ calculated for C₂₁H₁₂N₄O₄S: 417.42. found 417.29. LC/MS retention time: 2.21 minutes.

### Example 57: 3-[1,3-Dioxo-5-(1H-[1,2,3]triazol-4-yl)-1,3-dihydroisoindol-2-yl]-4′-fluorobiphenyl-4-carboxylic Acid

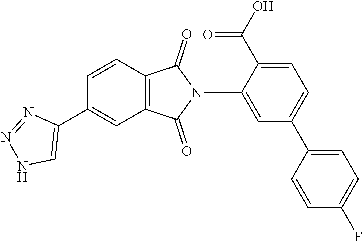 [SMILES: O=C(O)c1ccc(-c2ccc(F)cc2)cc1N1C(=O)c2ccc(-c3c[nH]nn3)cc2C1=O]

184. 3-[1,3-Dioxo-5-(1H-[1,2,3]triazol-4-yl)-1,3-dihydroisoindol-2-yl]-4′-fluorobiphenyl-4-carboxylic acid was prepared as described in synthetic procedure L from 4-(1H-1,2,3-triazol-4-yl)phthalic acid (Intermediate 5) and 3-amino-4′-fluorobiphenyl-4-carboxylic acid (was prepared from methyl 3-amino-4′-fluoro[1,1′-biphenyl]-4-carboxylate (Intermediate 14) as described in synthetic procedure B); MS m/z: (M+H)⁺ calculated for C₂₃H₁₃FN₄O₄: 429.38. found 429.19. LC/MS retention time: 2.26 minutes.

### Example 58: 3-[1,3-Dioxo-5-(1H-[1,2,3]triazol-4-yl)-1,3-dihydroisoindol-2-yl]-4′-methoxybiphenyl-4-carboxylic Acid

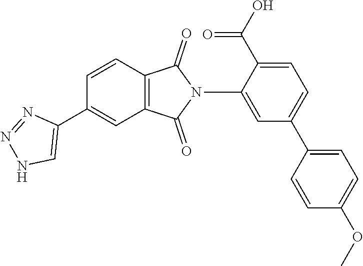 [SMILES: COc1ccc(-c2ccc(C(=O)O)c(N3C(=O)c4ccc(-c5c[nH]nn5)cc4C3=O)c2)cc1]

185. 3-[1,3-Dioxo-5-(1H-[1,2,3]triazol-4-yl)-1,3-dihydroisoindol-2-yl]-4′-methoxybiphenyl-4-carboxylic acid was prepared as described in synthetic procedure L from 4-(1H-1,2,3-triazol-4-yl)phthalic acid (Intermediate 5) and 3-amino-4′-methoxy[1,1′-biphenyl]-4-carboxylic acid; MS m/z: (M+H)⁺ calculated for C₂₄H₁₆N₄O₅: 441.42. found 441.20. LC/MS retention time: 2.21 minutes.

### Example 59: 3-[1,3-Dioxo-5-(1H-[1,2,3]triazol-4-yl)-1,3-dihydroisoindol-2-yl]biphenyl-4-carboxylic Acid

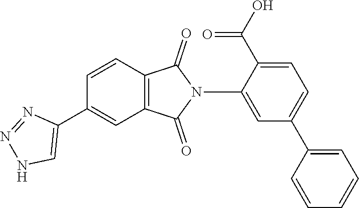 [SMILES: O=C(O)c1ccc(-c2ccccc2)cc1N1C(=O)c2ccc(-c3c[nH]nn3)cc2C1=O]

186. 3-[1,3-Dioxo-5-(1H-[1,2,3]triazol-4-yl)-1,3-dihydroisoindol-2-yl]biphenyl-4-carboxylic acid was prepared as described in synthetic procedure L from 4-(1H-1,2,3-triazol-4-yl)phthalic acid (Intermediate 5) and 3-amino[1,1′-biphenyl]-4-carboxylic acid; MS m/z: (M+H)⁺ calculated for C₂₃H₁₄N₄O₄: 411.39. found 411.36. LC/MS retention time: 2.29 minutes.

### Example 60: 2-[1,3-Dioxo-5-(1H-[1,2,3]triazol-4-yl)-1,3-dihydroisoindol-2-yl]-5-(3H-imidazol-4-yl)-benzoic acid

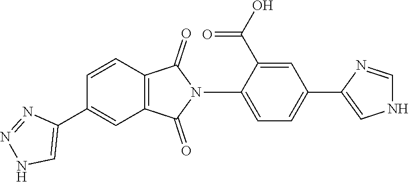 [SMILES: O=C(O)c1cc(-c2c[nH]cn2)ccc1N1C(=O)c2ccc(-c3c[nH]nn3)cc2C1=O]

187. 2-[1,3-Dioxo-5-(1H-[1,2,3]triazol-4-yl)-1,3-dihydroisoindol-2-yl]-5-(3H-imidazol-4-yl)-benzoic acid was prepared as described in synthetic procedure L from 4-(1H-1,2,3-triazol-4-yl)phthalic acid (Intermediate 5) and 2-amino-5-(1H-imidazol-4-yl)-benzoic acid (was prepared from methyl 2-amino-5-(1H-imidazol-4-yl)benzoate (Intermediate 6) as described in synthetic procedure E); MS m/z: (M+H)⁺ calculated for C₂₀H₁₂N₆O₄: 401.36. found 401.16. LC/MS retention time: 1.55 minutes.

### Example 61: 2-[1,3-Dioxo-5-(1H-[1,2,3]triazol-4-yl)-1,3-dihydroisoindol-2-yl]-5-(3-methyl-3H-imidazol-4-yl)-benzoic Acid

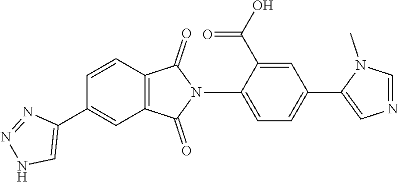 [SMILES: Cn1cncc1-c1ccc(N2C(=O)c3ccc(-c4c[nH]nn4)cc3C2=O)c(C(=O)O)c1]

188. 2-[1,3-Dioxo-5-(1H-[1,2,3]triazol-4-yl)-1,3-dihydroisoindol-2-yl]-5-(3-methyl-3H-imidazol-4-yl)-benzoic acid was prepared as described in synthetic procedure L starting from 4-(1H-1,2,3-triazol-4-yl)phthalic acid (Intermediate 5) and 2-amino-5-(1-methyl-1H-imidazol-5-yl)benzoic acid (was prepared as described in synthetic procedure C followed by synthetic procedure D followed by synthetic procedure E from methyl 2-amino-5-bromobenzoate and 5-iodo-1-methyl-1H-imidazole); MS m/z: (M+H)⁺ calculated for C₂₁H₁₄N₆O₄: 415.38. found 415.26. LC/MS retention time: 1.30 minutes.

### Example 62: 2-[1,3-Dioxo-5-(1H-[1,2,3]triazol-4-yl)-1,3-dihydroisoindol-2-yl]-5-pyridin-3-yl-benzoic acid

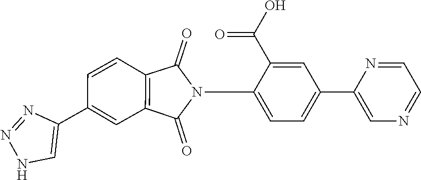 [SMILES: O=C(O)c1cc(-c2cnccn2)ccc1N1C(=O)c2ccc(-c3c[nH]nn3)cc2C1=O]

189. 2-[1,3-Dioxo-5-(1H-[1,2,3]triazol-4-yl)-1,3-dihydroisoindol-2-yl]-5-pyridin-3-yl-benzoic acid was prepared as described in synthetic procedure L from 4-(1H-1,2,3-triazol-4-yl)phthalic acid (Intermediate 5) and 2-amino-5-(pyridin-3-yl)benzoic acid (was prepared as described in synthetic procedure A followed by synthetic procedure E from methyl 2-amino-5-bromobenzoate and pyridine-3-boronic acid); MS m/z: (M+H)⁺ calculated for C₂₂H₁₃N₅O₄: 412.38. found 411.96. LC/MS retention time: 1.32 minutes.

### Example 63: 2-[1,3-Dioxo-5-(1H-[1,2,3]triazol-4-yl)-1,3-dihydroisoindol-2-yl]-5-pyrazin-2-yl-benzoic acid

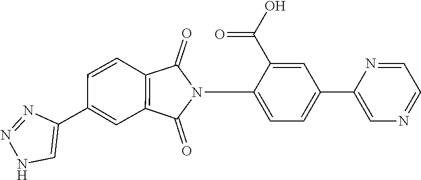 [SMILES: O=C(O)c1cc(-c2cnccn2)ccc1N1C(=O)c2ccc(-c3c[nH]nn3)cc2C1=O]

190. 2-[1,3-Dioxo-5-(1H-[1,2,3]triazol-4-yl)-1,3-dihydroisoindol-2-yl]-5-pyrazin-2-yl-benzoic acid was prepared as described in synthetic procedure L from 4-(1H-1,2,3-triazol-4-yl)phthalic acid (Intermediate 5) and 2-amino-5-(pyrazin-2-yl)benzoic acid (was prepared as described in synthetic procedure A followed by synthetic procedure E from methyl 2-amino-5-bromobenzoate and pyrazine-2-boronic acid); MS m/z: (M+H)⁺ calculated for C₂₁H₁₂N₆O₄: 413.37. found 413.16. LC/MS retention time: 1.71 minutes.

### Example 64: 2-[1,3-Dioxo-5-(1H-[1,2,3]triazol-4-yl)-1,3-dihydroisoindol-2-yl]-5-pyrimidin-5-yl-benzoic Acid

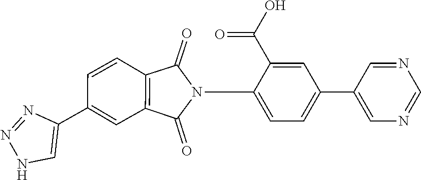 [SMILES: O=C(O)c1cc(-c2cncnc2)ccc1N1C(=O)c2ccc(-c3c[nH]nn3)cc2C1=O]

191. 2-[1,3-Dioxo-5-(1H-[1,2,3]triazol-4-yl)-1,3-dihydroisoindol-2-yl]-5-pyrimidin-5-yl-benzoic acid was prepared as described in synthetic procedure L from 4-(1H-1,2,3-triazol-4-yl)phthalic acid (Intermediate 5) and 2-amino-5-(pyrimidin-5-yl)benzoic acid (was prepared as described in synthetic procedure A followed by synthetic procedure E from methyl 2-amino-5-bromobenzoate and pyrimidine-5-boronic acid); MS m/z: (M+H)⁺ calculated for C₂₁H₁₂N₅O₄: 413.37. found 413.41. LC/MS retention time: 1.84 minutes.

### Example 65: 5-(6-Amino-pyridin-3-yl)-2-[1,3-dioxo-5-(1H-[1,2,3]triazol-4-yl)-1,3-dihydroisoindol-2-yl]-benzoic Acid

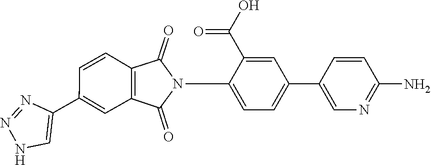 [SMILES: Nc1ccc(-c2ccc(N3C(=O)c4ccc(-c5c[nH]nn5)cc4C3=O)c(C(=O)O)c2)cn1]

192. 5-(6-Amino-pyridin-3-yl)-2-[1,3-dioxo-5-(1H-[1,2,3]triazol-4-yl)-1,3-dihydroisoindol-2-yl]-benzoic acid was prepared as described in synthetic procedure L from 4-(1H-1,2,3-triazol-4-yl)phthalic acid (Intermediate 5) and 2-amino-5-(6-aminopyridin-3-yl)benzoic acid (was prepared as described in synthetic procedure A followed by synthetic procedure E from methyl 2-amino-5-bromobenzoate and 6-aminopyridine-3-boronic acid pinacol ester); MS m/z: (M+H)⁺ calculated for C₂₂H₁₄N₆O₄: 427.39. found 427.27. LC/MS retention time: 1.38 minutes.

### Example 66: 3-[1,3-Dioxo-5-(1H-[1,2,3]triazol-4-yl)-1,3-dihydroisoindol-2-yl]biphenyl-4-carboxylic Acid 2-dimethylamino-ethyl ester

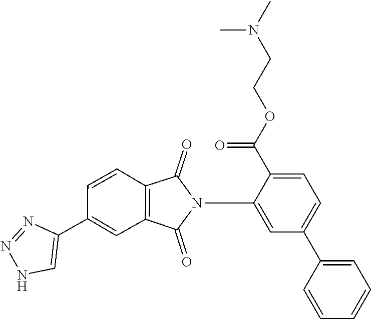 [SMILES: CN(C)CCOC(=O)c1ccc(-c2ccccc2)cc1N1C(=O)c2ccc(-c3c[nH]nn3)cc2C1=O]

193. 3-[1,3-Dioxo-5-(1H-[1,2,3]triazol-4-yl)-1,3-dihydroisoindol-2-yl]biphenyl-4-carboxylic acid 2-dimethylamino-ethyl ester was prepared as described in synthetic procedure P route A from 3-[1,3-dioxo-5-(1H-[1,2,3]triazol-4-yl)-1,3-dihydroisoindol-2-yl]biphenyl-4-carboxylic acid (see Example 59) and 2-dimethylaminoethanol; MS m/z: (M+H)⁺ calculated for C₂₂H₁₄N₆O₄: 482.52. found 482.20. LC/MS retention time: 1.96 minutes.

### Example 67: 3-[1,3-Dioxo-5-(1H-[1,2,3]triazol-4-yl)-1,3-dihydroisoindol-2-yl]biphenyl-4-carboxylic acid 2-morpholin-4-yl-ethyl ester

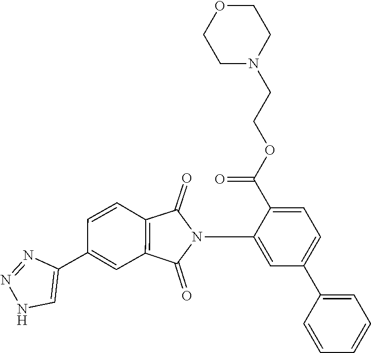 [SMILES: O=C(OCCN1CCOCC1)c1ccc(-c2ccccc2)cc1N1C(=O)c2ccc(-c3c[nH]nn3)cc2C1=O]

194. 3-[1,3-Dioxo-5-(1H-[1,2,3]triazol-4-yl)-1,3-dihydroisoindol-2-yl]biphenyl-4-carboxylic acid 2-morpholin-4-yl-ethyl ester was prepared as described in synthetic procedure P route A from 3-[1,3-dioxo-5-(1H-[1,2,3]triazol-4-yl)-1,3-dihydroisoindol-2-yl]biphenyl-4-carboxylic acid (see Example 59) and 4-(2-hydroxyethyl)morpholine; MS m/z: (M+H)⁺ calculated for C₂₉H₂₅N₅O₅: 524.55. found 524.52. LC/MS retention time: 1.98 minutes.

### Example 68: 3-[1,3-Dioxo-5-(1H-[1,2,3]triazol-4-yl)-1,3-dihydroisoindol-2-yl]biphenyl-4-carboxylic acid 2-pyrrolidin-1-yl-ethyl ester

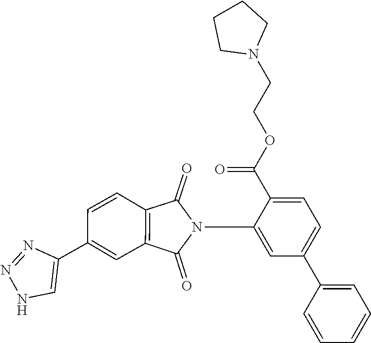 [SMILES: O=C(OCCN1CCCC1)c1ccc(-c2ccccc2)cc1N1C(=O)c2ccc(-c3c[nH]nn3)cc2C1=O]

195. 3-[1,3-Dioxo-5-(1H-[1,2,3]triazol-4-yl)-1,3-dihydroisoindol-2-yl]biphenyl-4-carboxylic acid 2-pyrrolidin-1-yl-ethyl ester was prepared as described in synthetic procedure P route A from 3-[1,3-dioxo-5-(1H-[1,2,3]triazol-4-yl)-1,3-dihydroisoindol-2-yl]biphenyl-4-carboxylic acid (see Example 59) and 1-(2-hydroxyethyl)pyrrolidine; MS m/z: (M+H)⁺ calculated for C₂₉H₂₅N₅O₄: 508.55. found 508.31. LC/MS retention time: 2.04 minutes.

### Example 69: 3-[1,3-Dioxo-5-(1H-[1,2,3]triazol-4-yl)-1,3-dihydroisoindol-2-yl]biphenyl-4-carboxylic acid 2-methoxy-ethyl ester

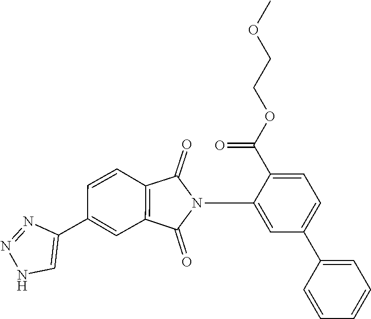 [SMILES: COCCOC(=O)c1ccc(-c2ccccc2)cc1N1C(=O)c2ccc(-c3c[nH]nn3)cc2C1=O]

196. 3-[1,3-Dioxo-5-(1H-[1,2,3]triazol-4-yl)-1,3-dihydroisoindol-2-yl]biphenyl-4-carboxylic acid 2-methoxy-ethyl ester was prepared as described in synthetic procedure P route A from 3-[1,3-dioxo-5-(1H-[1,2,3]triazol-4-yl)-1,3-dihydroisoindol-2-yl]biphenyl-4-carboxylic acid (see Example 59) and 2-methoxyethanol; MS m/z: (M+H)⁺ calculated for C₂₆H₂₀N₄O₅: 469.47. found 469.59. LC/MS retention time: 2.54 minutes.

### Example 70: 3-[1,3-Dioxo-5-(1H-[1,2,3]triazol-4-yl)-1,3-dihydroisoindol-2-yl]biphenyl-4-carboxylic acid 2,3-dihydroxy-propyl ester

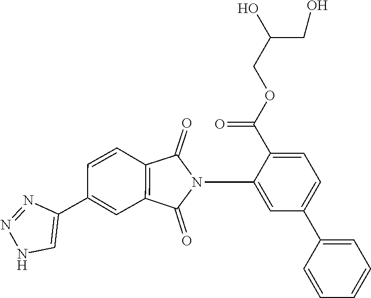 [SMILES: O=C(OCC(O)CO)c1ccc(-c2ccccc2)cc1N1C(=O)c2ccc(-c3c[nH]nn3)cc2C1=O]

197. 3-[1,3-Dioxo-5-(1H-[1,2,3]triazol-4-yl)-1,3-dihydroisoindol-2-yl]biphenyl-4-carboxylic acid 2,3-dihydroxy-propyl ester was prepared as described in synthetic procedure P route D from 3-[1,3-dioxo-5-(1H-[1,2,3]triazol-4-yl)-1,3-dihydroisoindol-2-yl]biphenyl-4-carboxylic acid (see Example 59) and glycerol; MS m/z: (M+H)⁺ calculated for C₂₆H₂₀N₄O₆: 485.47. found 485.50. LC/MS retention time: 1.91 minutes.

### Example 71: 2-[1,3-Dioxo-5-(1H-[1,2,3]triazol-4-yl)-1,3-dihydroisoindol-2-yl]-5-(1H-pyrazol-4-yl)-benzoic Acid

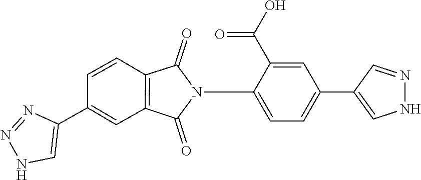 [SMILES: O=C(O)c1cc(-c2cn[nH]c2)ccc1N1C(=O)c2ccc(-c3c[nH]nn3)cc2C1=O]

198. 2-[1,3-Dioxo-5-(1H-[1,2,3]triazol-4-yl)-1,3-dihydroisoindol-2-yl]-5-(1H-pyrazol-4-yl)-benzoic acid was prepared as described in synthetic procedure L from 4-(1H-1,2,3-triazol-4-yl)phthalic acid (Intermediate 5) and 2-amino-5-(1H-pyrazol-4-yl)-benzoic acid (was prepared as described in synthetic procedure A followed by synthetic procedure E from methyl 2-amino-5-bromobenzoate and 1H-pyrazole-4-boronic acid); MS m/z: (M+H)⁺ calculated for C₂₀H₁₂N₆O₄: 401.36. found 401.16. LC/MS retention time: 1.58 minutes.

### Example 72: 3-[1,3-Dioxo-5-(1H-[1,2,3]triazol-4-yl)-1,3-dihydroisoindol-2-yl]biphenyl-4-carboxylic Acid 2-amino-ethyl ester

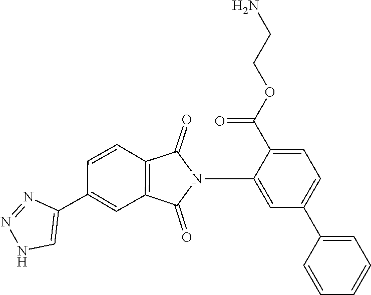 [SMILES: NCCOC(=O)c1ccc(-c2ccccc2)cc1N1C(=O)c2ccc(-c3c[nH]nn3)cc2C1=O]

199. 3-[1,3-Dioxo-5-(1H-[1,2,3]triazol-4-yl)-1,3-dihydroisoindol-2-yl]biphenyl-4-carboxylic acid 2-amino-ethyl ester was prepared as described in synthetic procedure P route E from 3-[1,3-dioxo-5-(1H-[1,2,3]triazol-4-yl)-1,3-dihydroisoindol-2-yl]biphenyl-4-carboxylic acid (see Example 59) and N-Boc-ethanolamine; MS m/z: (M+H)⁺ calculated for C₂₅H₁₉N₅O₄: 454.46. found 453.98. LC/MS retention time: 2.00 minutes.

### Example 73: 3-[1,3-Dioxo-5-(1H-[1,2,3]triazol-4-yl)-1,3-dihydroisoindol-2-yl]biphenyl-4-carboxylic acid (2S)-2-amino-2-carboxy-ethyl ester

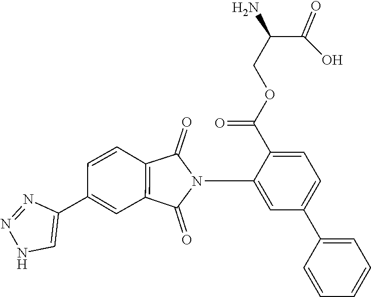 [SMILES: N[C@H](COC(=O)c1ccc(-c2ccccc2)cc1N1C(=O)c2ccc(-c3c[nH]nn3)cc2C1=O)C(=O)O]

200. 3-[1,3-Dioxo-5-(1H-[1,2,3]triazol-4-yl)-1,3-dihydroisoindol-2-yl]biphenyl-4-carboxylic acid (2S)-2-amino-2-carboxy-ethyl ester was prepared as described in synthetic procedure P route E from 3-[1,3-dioxo-5-(1H-[1,2,3]triazol-4-yl)-1,3-dihydroisoindol-2-yl]biphenyl-4-carboxylic acid (see Example 59) and N-Boc-L-serine; MS m/z: (M+H)⁺ calculated for C₂₆H₁₉N₅O₆: 498.47. found 498.40. LC/MS retention time: 2.05 minutes.

### Example 74: 3-[1,3-Dioxo-5-(1H-[1,2,3]triazol-4-yl)-1,3-dihydroisoindol-2-yl]biphenyl-4-carboxylic acid 2-{[(2S)-2-amino-3-methylbutanoyl]oxy}ethyl ester

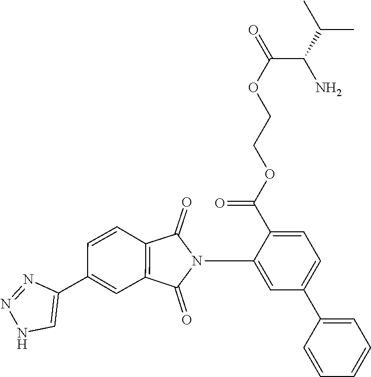 [SMILES: CC(C)[C@H](N)C(=O)OCCOC(=O)c1ccc(-c2ccccc2)cc1N1C(=O)c2ccc(-c3c[nH]nn3)cc2C1=O]

201. 3-[1,3-Dioxo-5-(1H-[1,2,3]triazol-4-yl)-1,3-dihydroisoindol-2-yl]biphenyl-4-carboxylic acid 2-{[(2S)-2-amino-3-methylbutanoyl]oxy}ethyl ester was prepared as described in synthetic procedure P route E from 3-[1,3-dioxo-5-(1H-[1,2,3]triazol-4-yl)-1,3-dihydroisoindol-2-yl]biphenyl-4-carboxylic acid (see Example 59) and Boc-L-valine 2-hydroxyethyl ester (was prepared from Boc-L-valine methyl ester as described in Curr. Protoc. Nucleic Acid Chem., Chapter: Unit 15.4); MS m/z: (M+H)⁺ calculated for C₂₉H₂₄N₄O₆: 554.58. found 554.20. LC/MS retention time: 2.27 minutes.

### Example 75: 2-[2-Carboxy-4-(1H-imidazol-4-yl)-phenyl]-1,3-dioxo-2,3-dihydro-1H-isoindole-5-carboxylic Acid

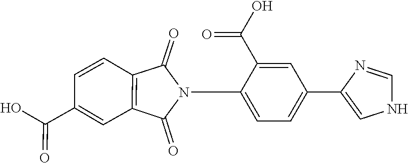 [SMILES: O=C(O)c1ccc2c(c1)C(=O)N(c1ccc(-c3c[nH]cn3)cc1C(=O)O)C2=O]

202. 2-[2-Carboxy-4-(1H-imidazol-4-yl)-phenyl]-1,3-dioxo-2,3-dihydro-1H-isoindole-5-carboxylic acid was prepared as described in synthetic procedure K from trimellitic anhydride and 2-amino-5-(1H-imidazol-4-yl)benzoic acid (was prepared from methyl 2-amino-5-(1H-imidazol-4-yl)benzoate (Intermediate 6) as described in synthetic procedure E); MS m/z: (M+H)⁺ calculated for C₁₉H₁₁N₃O₆: 378.32. found 378.30. LC/MS retention time: 1.10 minutes.

### Example 76: 2-[2-Carboxy-4-(3-methyl-3H-imidazol-4-yl)-phenyl]-1,3-dioxo-2,3-dihydro-1H-isoindole-5-carboxylic Acid

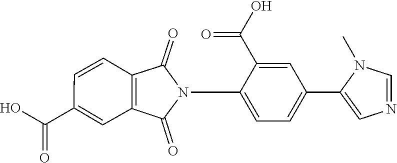 [SMILES: Cn1cncc1-c1ccc(N2C(=O)c3ccc(C(=O)O)cc3C2=O)c(C(=O)O)c1]

203. 2-[2-Carboxy-4-(3-methyl-3H-imidazol-4-yl)-phenyl]-1,3-dioxo-2,3-dihydro-1H-isoindole-5-carboxylic acid was prepared as described in synthetic procedure K from trimellitic anhydride and 2-amino-5-(1-methyl-1H-imidazol-5-yl)benzoic acid (was prepared as described in synthetic procedure C followed by synthetic procedure D followed by synthetic procedure E from methyl 2-amino-5-bromobenzoate and 5-iodo-1-methyl-1H-imidazole); MS m/z: (M+H)⁺ calculated for C₂₀H₁₃N₃O₆: 392.34. found 392.20. LC/MS retention time: 1.12 minutes.

### Example 77: 2-[4-(1H-Imidazol-4-yl)-2-(2-methylaminoethoxycarbonyl)-phenyl]-1,3-dioxo-2,3-dihydro-1H-isoindole-5-carboxylic Acid

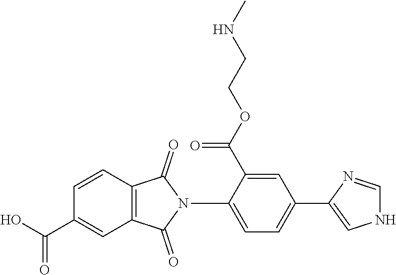 [SMILES: CNCCOC(=O)c1cc(-c2c[nH]cn2)ccc1N1C(=O)c2ccc(C(=O)O)cc2C1=O]

204. 2-[4-(1H-Imidazol-4-yl)-2-(2-methylaminoethoxycarbonyl)-phenyl]-1,3-dioxo-2,3-dihydro-1H-isoindole-5-carboxylic acid was prepared as described in synthetic procedure AB from 2-{5-[(benzyloxy)carbonyl]-1,3-dioxoisoindolin-2-yl}-5-(1H-imidazol-4-yl)benzoic acid (Intermediate 6a) and N-Boc-N-methyl-ethanolamine; MS m/z: (M+H)⁺ calculated for C₂₂H₁₈N₄O₆: 435.41. found 435.40. LC/MS retention time: 1.06 minutes.

### Example 78: 4-[1,3-Dioxo-5-(1H-[1,2,3]triazol-4-yl)-1,3-dihydroisoindol-2-yl]biphenyl-3-carboxylic acid 2-dimethylamino-ethyl ester

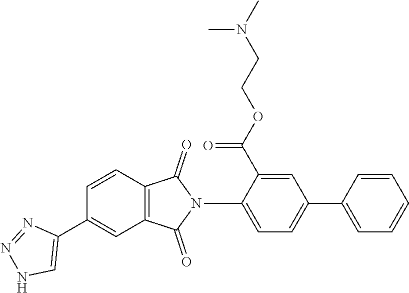 [SMILES: CN(C)CCOC(=O)c1cc(-c2ccccc2)ccc1N1C(=O)c2ccc(-c3c[nH]nn3)cc2C1=O]

205. 4-[1,3-Dioxo-5-(1H-[1,2,3]triazol-4-yl)-1,3-dihydroisoindol-2-yl]biphenyl-3-carboxylic acid 2-dimethylamino-ethyl ester was prepared as described in synthetic procedure P route A from 4-[1,3-dioxo-5-(1H-[1,2,3]triazol-4-yl)-1,3-dihydroisoindol-2-yl]biphenyl-3-carboxylic acid (see Example 31) and 2-dimethylaminoethanol; MS m/z: (M+H)⁺ calculated for C₂₇H₂₃N₅O₄: 482.52. found 482.20. LC/MS retention time: 1.87 minutes.

### Example 79: 4-[1,3-Dioxo-5-(1H-[1,2,3]triazol-4-yl)-1,3-dihydroisoindol-2-yl]biphenyl-3-carboxylic acid 2-pyrrolidin-1-yl-ethyl ester

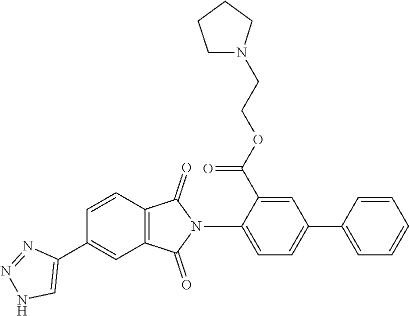 [SMILES: O=C(OCCN1CCCC1)c1cc(-c2ccccc2)ccc1N1C(=O)c2ccc(-c3c[nH]nn3)cc2C1=O]

206. 4-[1,3-Dioxo-5-(1H-[1,2,3]triazol-4-yl)-1,3-dihydroisoindol-2-yl]biphenyl-3-carboxylic acid 2-pyrrolidin-1-yl-ethyl ester was prepared as described in synthetic procedure P route A from 4-[1,3-dioxo-5-(1H-[1,2,3]triazol-4-yl)-1,3-dihydroisoindol-2-yl]biphenyl-3-carboxylic acid (see Example 31) and 1-(2-hydroxyethyl)pyrrolidine; MS m/z: (M+H)⁺ calculated for C₂₉H₂₅N₅O₄: 508.55. found 508.30. LC/MS retention time: 1.94 minutes.

### Example 80: 4-[1,3-Dioxo-5-(1H-[1,2,3]triazol-4-yl)-1,3-dihydroisoindol-2-yl]biphenyl-3-carboxylic acid 2-morpholin-4-yl-ethyl ester

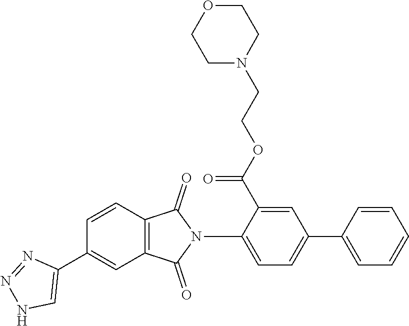 [SMILES: O=C(OCCN1CCOCC1)c1cc(-c2ccccc2)ccc1N1C(=O)c2ccc(-c3c[nH]nn3)cc2C1=O]

207. 4-[1,3-Dioxo-5-(1H-[1,2,3]triazol-4-yl)-1,3-dihydroisoindol-2-yl]biphenyl-3-carboxylic acid 2-morpholin-4-yl-ethyl ester was prepared as described in synthetic procedure P route A from 4-[1,3-dioxo-5-(1H-[1,2,3]triazol-4-yl)-1,3-dihydroisoindol-2-yl]biphenyl-3-carboxylic acid (see Example 31) and 4-(2-hydroxyethyl)morpholine; MS m/z: (M+H)⁺ calculated for C₂₉H₂₅N₅O₅: 524.55. found 524.50. LC/MS retention time: 1.91 minutes.

### Example 81: 4-[1,3-Dioxo-5-(1H-[1,2,3]triazol-4-yl)-1,3-dihydroisoindol-2-yl]biphenyl-3-carboxylic acid 2-methylamino-ethyl ester

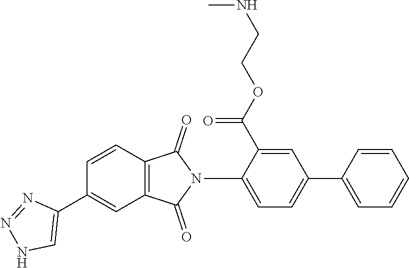 [SMILES: CNCCOC(=O)c1cc(-c2ccccc2)ccc1N1C(=O)c2ccc(-c3c[nH]nn3)cc2C1=O]

208. 4-[1,3-Dioxo-5-(1H-[1,2,3]triazol-4-yl)-1,3-dihydroisoindol-2-yl]biphenyl-3-carboxylic acid 2-methylamino-ethyl ester was prepared as described in synthetic procedure P route E from 4-[1,3-dioxo-5-(1H-[1,2,3]triazol-4-yl)-1,3-dihydroisoindol-2-yl]biphenyl-3-carboxylic acid (see Example 31) and N-Boc-N-methyl-ethanolamine; MS m/z: (M+H)⁺ calculated for C₂₆H₂₁N₅O₄: 468.49. found 468.10. LC/MS retention time: 1.90 minutes.

### Example 82: 3′,4′-Difluoro-3-[1,3-dioxo-5-(1H-[1,2,3]triazol-4-yl)-1,3-dihydroisoindol-2-yl]biphenyl-4-carboxylic Acid

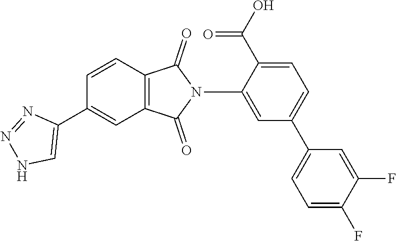 [SMILES: O=C(O)c1ccc(-c2ccc(F)c(F)c2)cc1N1C(=O)c2ccc(-c3c[nH]nn3)cc2C1=O]

209. 3′,4′-Difluoro-3-[1,3-dioxo-5-(1H-[1,2,3]triazol-4-yl)-1,3-dihydroisoindol-2-yl]biphenyl-4-carboxylic acid was prepared as described in synthetic procedure L from 4-(1H-1,2,3-triazol-4-yl)phthalic acid (Intermediate 5) and 3-amino-3′,4′-difluoro[1,1′-biphenyl]-4-carboxylic acid (Intermediate 1d) as described in synthetic procedure B); MS m/z: (M+H)⁺ calculated for C₂₃H₁₂F₂N₄O₄: 447.37. found 447.10. LC/MS retention time: 2.21 minutes.

### Example 83: 2-(2-Hydroxy-5-phenylpyridin-3-yl)-5-(1H-[1,2,3]triazol-4-yl)-isoindole-1,3-dion

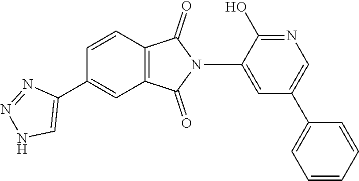 [SMILES: O=C1c2ccc(-c3c[nH]nn3)cc2C(=O)N1c1cc(-c2ccccc2)cnc1O]

210. 2-(2-Hydroxy-5-phenylpyridin-3-yl)-5-(1H-[1,2,3]triazol-4-yl)-isoindole-1,3-dione was prepared as described in synthetic procedure L from 4-(1H-1,2,3-triazol-4-yl)phthalic acid (Intermediate 5) and 3-amino-2-hydroxy-5-phenylpyridine; MS m/z: (M+H)⁺ calculated for C₂₁H₁₃N₅O₃: 384.37. found 384.30. LC/MS retention time: 1.86 minutes.

### Example 84: 2-(4-Phenylpyridin-2-yl)-5-(1H-[1,2,3]triazol-4-yl)-isoindole-1,3-dione

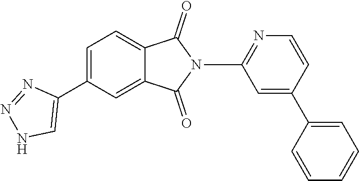 [SMILES: O=C1c2ccc(-c3c[nH]nn3)cc2C(=O)N1c1cc(-c2ccccc2)ccn1]

211. 2-(4-Phenylpyridin-2-yl)-5-(1H-[1,2,3]triazol-4-yl)-isoindole-1,3-dione was prepared as described in synthetic procedure L from 4-(1H-1,2,3-triazol-4-yl)phthalic acid (Intermediate 5) and 2-amino-4-phenylpyridine; MS m/z: (M+H)⁺ calculated for C₂₁H₁₃N₅O₂: 368.10. found 368.37. LC/MS retention time: 2.15 minutes.

### Example 85: 2-{1,3-Dioxo-5-(1H-[1,2,3]triazol-4-yl)-2,3-dihydro-1H-isoindol-2-yl}-4-phenylpyridine N-oxide

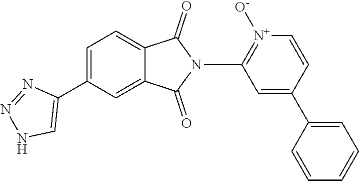 [SMILES: O=C1c2ccc(-c3c[nH]nn3)cc2C(=O)N1c1cc(-c2ccccc2)cc[n+]1[O-]]

212. 2-{1,3-Dioxo-5-(1H-[1,2,3]triazol-4-yl)-2,3-dihydro-1H-isoindol-2-yl}-4-phenylpyridine N-oxide was prepared as described in synthetic procedure AC from 2-(4-phenylpyridin-3-yl)-5-(1H-[1,2,3]triazol-4-yl)-isoindole-1,3-dione (see Example 84); MS m/z: (M+H)⁺ calculated for C₂₁H₁₃N₅O₃: 384.37. found 384.00. LC/MS retention time: 1.94 minutes.

### Example 86: 2-(5-Phenylpyridin-2-yl)-5-(1H-[1,2,3]triazol-4-yl)-isoindole-1,3-dione

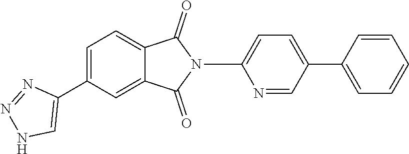 [SMILES: O=C1c2ccc(-c3c[nH]nn3)cc2C(=O)N1c1ccc(-c2ccccc2)cn1]

213. 2-(5-Phenylpyridin-2-yl)-5-(1H-[1,2,3]triazol-4-yl)-isoindole-1,3-dione was prepared as described in synthetic procedure L from 4-(1H-1,2,3-triazol-4-yl)phthalic acid (Intermediate 5) and 2-amino-5-phenylpyridine; MS m/z: (M+H)⁺ calculated for C₂₁H₁₃N₅O₂: 368.37. found 368.10. LC/MS retention time: 2.24 minutes.

### Example 87: 2-[4-(4-Fluorophenyl)-pyridin-2-yl]-5-(1H-[1,2,3]triazol-4-yl)-isoindole-1,3-dione

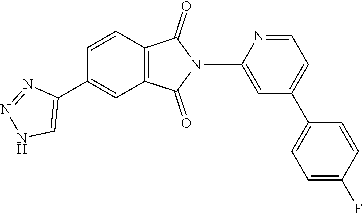 [SMILES: O=C1c2ccc(-c3c[nH]nn3)cc2C(=O)N1c1cc(-c2ccc(F)cc2)ccn1]

214. 2-[4-(4-Fluorophenyl)-pyridin-2-yl]-5-(1H-[1,2,3]triazol-4-yl)-isoindole-1,3-dione was prepared as described in synthetic procedure L from 4-(1H-1,2,3-triazol-4-yl)phthalic acid (Intermediate 5) and 4-(4-fluorophenyl)pyridin-2-amine; MS m/z: (M+H)⁺ calculated for C₂₁H₁₂FN₅O₂: 386.36. found 386.10. LC/MS retention time: 2.30 minutes.

### Example 88: 2-(6-Methyl-4-phenylpyridin-2-yl)-5-(1H-[1,2,3]triazol-4-yl)-isoindole-1,3-dione

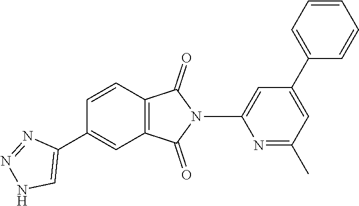 [SMILES: Cc1cc(-c2ccccc2)cc(N2C(=O)c3ccc(-c4c[nH]nn4)cc3C2=O)n1]

215. 2-(6-Methyl-4-phenylpyridin-2-yl)-5-(1H-[1,2,3]triazol-4-yl)-isoindole-1,3-dione was prepared as described in synthetic procedure L from 4-(1H-1,2,3-triazol-4-yl)phthalic acid (Intermediate 5) and 6-methyl-4-phenylpyridin-2-amine; MS m/z: (M+H)⁺ calculated for C₂₂H₁₅N₅O₂: 386.36. found 386.20. LC/MS retention time: 2.36 minutes.

### Example 89: 2-(2-Hydroxy-6-phenylpyridin-3-yl)-5-(1H-[1,2,3]triazol-4-yl)-isoindole-1,3-dione

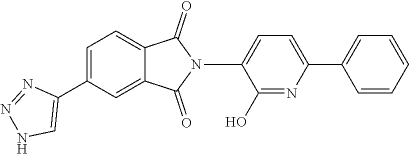 [SMILES: O=C1c2ccc(-c3c[nH]nn3)cc2C(=O)N1c1ccc(-c2ccccc2)nc1O]

216. 2-(2-Hydroxy-6-phenylpyridin-3-yl)-5-(1H-[1,2,3]triazol-4-yl)-isoindole-1,3-dione was prepared as described in synthetic procedure L from 4-(1H-1,2,3-triazol-4-yl)phthalic acid (Intermediate 5) and 3-amino-2-hydroxy-6-phenylpyridine; MS m/z: (M+H)⁺ calculated for C₂₁H₁₃N₅O₃: 384.37. found 384.00. LC/MS retention time: 1.99 minutes.

### Example 90: 2-[4-(2,4-Difluoro-phenyl)-5-methyl-pyridin-2-yl]-5-(1H-[1,2,3]triazol-4-yl)-isoindole-1,3-dione

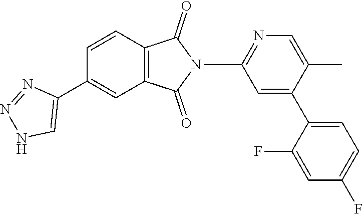 [SMILES: Cc1cnc(N2C(=O)c3ccc(-c4c[nH]nn4)cc3C2=O)cc1-c1ccc(F)cc1F]

217. 2-[4-(2,4-Difluoro-phenyl)-5-methyl-pyridin-2-yl]-5-(1H-[1,2,3]triazol-4-yl)-isoindole-1,3-dione was prepared as described in synthetic procedure L from 4-(1H-1,2,3-triazol-4-yl)phthalic acid (Intermediate 5) and 2-amino-4-(2,4-difluorophenyl)-5-methylpyridine (Intermediate 9); MS m/z: (M+H)⁺ calculated for C₂₂H₁₃N₅O₂: 418.38. found 418.30. LC/MS retention time: 2.15 minutes.

### Example 91: 3-[1-Oxo-6-(1H-[1,2,3]triazol-4-yl)-1,3-dihydroisoindol-2-yl]biphenyl-4-carboxylic acid methyl ester

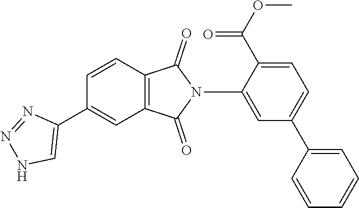 [SMILES: COC(=O)c1ccc(-c2ccccc2)cc1N1C(=O)c2ccc(-c3c[nH]nn3)cc2C1=O]

218. 3-[1-Oxo-6-(1H-[1,2,3]triazol-4-yl)-1,3-dihydroisoindol-2-yl]biphenyl-4-carboxylic acid methyl ester was prepared as described in synthetic procedure Y followed by synthetic procedure Z from 3-(6-bromo-1-oxo-1,3-dihydroisoindol-2-yl)biphenyl-4-carboxylic acid methyl ester (Intermediate 3a), ethynyltrimethylsilane and trimethylsilyl azide; MS m/z: (M+H)⁺ calculated for C₂₄H₁N₄O₃: 411.43. found 411.10. LC/MS retention time: 2.42 minutes.

### Example 92: 3-[1-Oxo-6-(1H-[1,2,3]triazol-4-yl)-1,3-dihydroisoindol-2-yl]biphenyl-4-carboxylic Acid

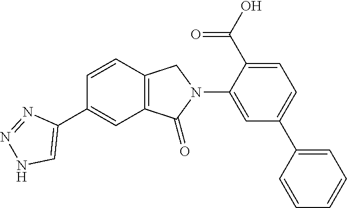 [SMILES: O=C(O)c1ccc(-c2ccccc2)cc1N1Cc2ccc(-c3c[nH]nn3)cc2C1=O]

219. 3-[1-Oxo-6-(1H-[1,2,3]triazol-4-yl)-1,3-dihydroisoindol-2-yl]biphenyl-4-carboxylic acid was prepared as described in synthetic procedure Z step 2 from 3-[1-oxo-6-(1H-[1,2,3]triazol-4-yl)-1,3-dihydroisoindol-2-yl]biphenyl-4-carboxylic acid methyl ester (see Example 91); MS m/z: (M+H)⁺ calculated for C₂₃H₁₆N₄O₃: 397.41. found 397.30. LC/MS retention time: 2.17 minutes.

### Example 93: 3-[1,3-Dioxo-5-(1H-[1,2,3]triazol-4-yl)-1,3-dihydroisoindol-2-yl]biphenyl-4-carboxylic acid amide

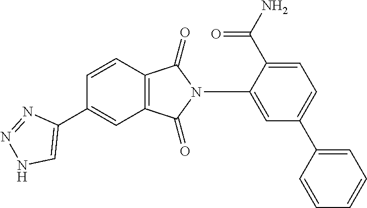 [SMILES: NC(=O)c1ccc(-c2ccccc2)cc1N1C(=O)c2ccc(-c3c[nH]nn3)cc2C1=O]

220. 3-[1,3-Dioxo-5-(1H-[1,2,3]triazol-4-yl)-1,3-dihydroisoindol-2-yl]biphenyl-4-carboxylic acid amide was prepared as described in synthetic procedure P from 3-[1,3-dioxo-5-(1H-[1,2,3]triazol-4-yl)-1,3-dihydroisoindol-2-yl]biphenyl-4-carboxylic acid (see Example 59) and NH₄Cl; MS m/z: (M+H)⁺ calculated for C₂₃H₁₅N₅O₃: 410.41. found 410.20. LC/MS retention time: 1.99 minutes.

### Example 94: 4-[1,3-Dioxo-5-(1H-tetrazol-5-yl)-1,3-dihydroisoindol-2-yl]biphenyl-3-carboxylic Acid

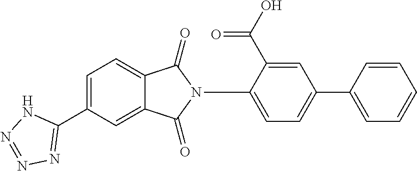 [SMILES: O=C(O)c1cc(-c2ccccc2)ccc1N1C(=O)c2ccc(-c3nnn[nH]3)cc2C1=O]

221. 4-[1,3-Dioxo-5-(1H-tetrazol-5-yl)-1,3-dihydroisoindol-2-yl]biphenyl-3-carboxylic acid was prepared as described in synthetic procedure M from 4-cyano-1,2-benzenedicarboxylic acid, 4-amino[1,1′-biphenyl]-3-carboxylic acid (Intermediate 13) and sodium azide; MS m/z: (M+H)⁺ calculated for C₂₂H₁₃N₅O₄: 412.38. found 412.26. LC/MS retention time: 2.12 minutes.

### Example 95: 3-[1,3-Dioxo-5-(1H-tetrazol-5-yl)-1,3-dihydroisoindol-2-yl]biphenyl-4-carboxylic Acid

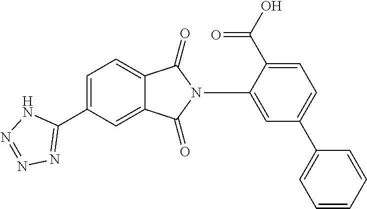 [SMILES: O=C(O)c1ccc(-c2ccccc2)cc1N1C(=O)c2ccc(-c3nnn[nH]3)cc2C1=O]

222. 3-[1,3-Dioxo-5-(1H-tetrazol-5-yl)-1,3-dihydroisoindol-2-yl]biphenyl-4-carboxylic acid was prepared as described in synthetic procedure M from 4-cyano-1,2-benzenedicarboxylic acid, sodium azide and 3-amino[1,1′-biphenyl]-4-carboxylic acid; (M+H)⁺ calculated for C₂₂H₁₃N₅O₄: 412.38. found 411.96. LC/MS retention time: 2.11 minutes.

### Example 96: 3-[1,3-Dioxo-5-(1H-tetrazol-5-yl)-1,3-dihydroisoindol-2-yl]biphenyl-4-carboxylic acid isopropyl ester

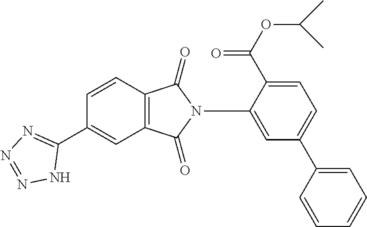 [SMILES: CC(C)OC(=O)c1ccc(-c2ccccc2)cc1N1C(=O)c2ccc(-c3nnn[nH]3)cc2C1=O]

223. 3-[1,3-Dioxo-5-(1H-tetrazol-5-yl)-1,3-dihydroisoindol-2-yl]biphenyl-4-carboxylic acid isopropyl ester was prepared as described in synthetic procedure P route A from 3-[1,3-dioxo-5-(1H-tetrazol-5-yl)-1,3-dihydroisoindol-2-yl]biphenyl-4-carboxylic acid (see Example 95) and isopropanol; MS m/z: (M+H)⁺ calculated for C₂₅H₁₅N₅O₄: 454.46. found 454.00. LC/MS retention time: 2.86 minutes.

### Example 97: 3-[1,3-Dioxo-5-(1H-tetrazol-5-yl)-1,3-dihydroisoindol-2-yl]biphenyl-4-carboxylic acid 2-dimethylamino-ethyl ester

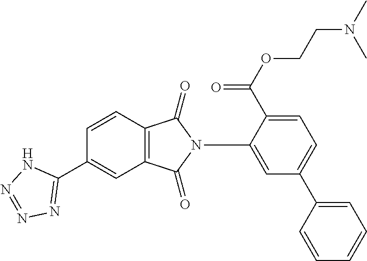 [SMILES: CN(C)CCOC(=O)c1ccc(-c2ccccc2)cc1N1C(=O)c2ccc(-c3nnn[nH]3)cc2C1=O]

224. 3-[1,3-Dioxo-5-(1H-tetrazol-5-yl)-1,3-dihydroisoindol-2-yl]biphenyl-4-carboxylic acid 2-dimethylamino-ethyl ester was prepared as described in synthetic procedure P route A from 3-[1,3-dioxo-5-(1H-tetrazol-5-yl)-1,3-dihydroisoindol-2-yl]biphenyl-4-carboxylic acid (see Example 95) and 2-dimethylaminoethanol; MS m/z: (M+H)⁺ calculated for C₂₆H₂₂N₆O₄: 483.50. found 483.40. LC/MS retention time: 2.27 minutes.

### Example 98: 3-[1,3-Dioxo-5-(1H-tetrazol-5-yl)-1,3-dihydroisoindol-2-yl]biphenyl-4-carboxylic acid amide

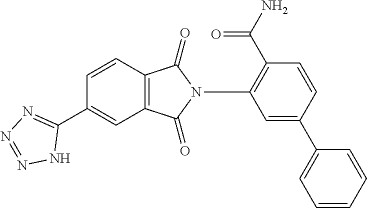 [SMILES: NC(=O)c1ccc(-c2ccccc2)cc1N1C(=O)c2ccc(-c3nnn[nH]3)cc2C1=O]

225. 3-[1,3-Dioxo-5-(1H-tetrazol-5-yl)-1,3-dihydroisoindol-2-yl]biphenyl-4-carboxylic acid amide was prepared as described in synthetic procedure P from 3-[1,3-dioxo-5-(1H-tetrazol-5-yl)-1,3-dihydroisoindol-2-yl]biphenyl-4-carboxylic acid (see Example 95) and NH₄Cl; MS m/z: (M+H)⁺ calculated for C₂₂H₁₄N₆O₃: 411.40. found 411.40. LC/MS retention time: 2.28 minutes.

### Example 99: 3′,4′-Difluoro-3-[1-oxo-6-(1H-tetrazol-5-yl)-1,3-dihydroisoindol-2-yl]biphenyl-4-carboxylic acid methyl ester

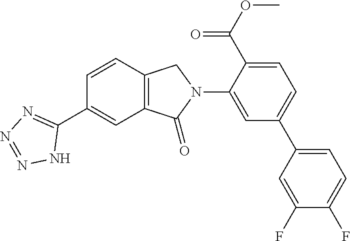 [SMILES: COC(=O)c1ccc(-c2ccc(F)c(F)c2)cc1N1Cc2ccc(-c3nnn[nH]3)cc2C1=O]

226. 3′,4′-Difluoro-3-[1-oxo-6-(1H-tetrazol-5-yl)-1,3-dihydroisoindol-2-yl]biphenyl-4-carboxylic acid methyl ester was prepared as described in synthetic procedure T from 3-(6-bromo-1-oxo-1,3-dihydroisoindol-2-yl)-3′,4′-difluorobiphenyl-4-carboxylic acid methyl ester (Intermediate 2) and sodium azide; MS m/z: (M+H)⁺ calculated for C₂₃H₁₅F₂N₅O₃: 448.40. found 448.00. LC/MS retention time: 2.59 minutes.

### Example 100: 3′,4′-Difluoro-3-[1-oxo-6-(1H-tetrazol-5-yl)-1,3-dihydroisoindol-2-yl]biphenyl-4-carboxylic Acid

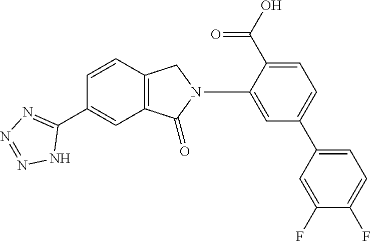 [SMILES: O=C(O)c1ccc(-c2ccc(F)c(F)c2)cc1N1Cc2ccc(-c3nnn[nH]3)cc2C1=O]

227. 3′,4′-Difluoro-3-[1-oxo-6-(1H-tetrazol-5-yl)-1,3-dihydroisoindol-2-yl]biphenyl-4-carboxylic acid was prepared as described in synthetic procedure T from 3′,4′-difluoro-3-[1-oxo-6-(1H-tetrazol-5-yl)-1,3-dihydroisoindol-2-yl]biphenyl-4-carboxylic acid methyl ester (see Example 99); MS m/z: (M+H)⁺ calculated for C₂₂H₁₃F₂N₅O₃: 434.38. found 434.20. LC/MS retention time: 2.40 minutes.

### Example 101: 3′,4′-Difluoro-3-[1-oxo-6-(1H-tetrazol-5-yl)-1,3-dihydroisoindol-2-yl]biphenyl-4-carboxylic acid amide

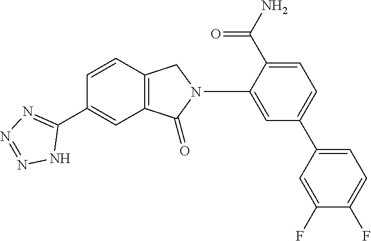 [SMILES: NC(=O)c1ccc(-c2ccc(F)c(F)c2)cc1N1Cc2ccc(-c3nnn[nH]3)cc2C1=O]

228. 3′,4′-Difluoro-3-[1-oxo-6-(1H-tetrazol-5-yl)-1,3-dihydroisoindol-2-yl]biphenyl-4-carboxylic acid amide was prepared as described in synthetic procedure P from 3′,4′-difluoro-3-[1-oxo-6-(1H-tetrazol-5-yl)-1,3-dihydroisoindol-2-yl]biphenyl-4-carboxylic acid (see Example 100) and NH₄Cl; MS m/z: (M+H)⁺ calculated for C₂₂H₁₄F₂N₆O₂: 433.39. found 433.30. LC/MS retention time: 2.44 minutes.

### Example 102: 2-(4-Methoxycarbonylbiphenyl-3-yl)-3-oxo-2,3-dihydro-1H-isoindole-5-carboxylic Acid

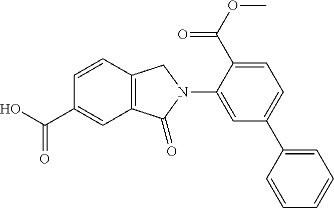 [SMILES: COC(=O)c1ccc(-c2ccccc2)cc1N1Cc2ccc(C(=O)O)cc2C1=O]

229. 2-(4-Methoxycarbonylbiphenyl-3-yl)-3-oxo-2,3-dihydro-1H-isoindole-5-carboxylic acid was prepared as described in synthetic procedure R from 3-(6-bromo-1-oxo-1,3-dihydroisoindol-2-yl)biphenyl-4-carboxylic acid methyl ester (Intermediate 3a); MS m/z: (M+H)⁺ calculated for C₂₃H₁₇NO₅: 388.40. found 388.20. LC/MS retention time: 2.56 minutes.

### Example 103: 3-(6-Methanesulfonylaminocarbonyl-1-oxo-1,3-dihydroisoindol-2-yl)biphenyl-4-carboxylic acid methyl ester

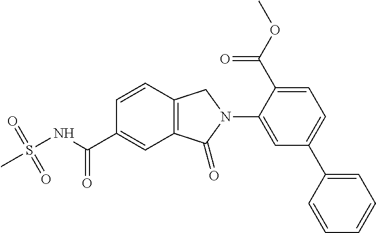 [SMILES: COC(=O)c1ccc(-c2ccccc2)cc1N1Cc2ccc(C(=O)NS(C)(=O)=O)cc2C1=O]

230. 3-(6-Methanesulfonylaminocarbonyl-1-oxo-1,3-dihydroisoindol-2-yl)biphenyl-4-carboxylic acid methyl ester was prepared as described in synthetic procedure S from 2-(4-methoxycarbonylbiphenyl-3-yl)-3-oxo-2,3-dihydro-1H-isoindole-5-carboxylic acid (see Example 102) and methanesulfonamide; MS m/z: (M+H)⁺ calculated for C₂₄H₂₀N₂O₆S: 464.50. found 465.10. LC/MS retention time: 2.59 minutes.

### Example 104: 3-(6-Methanesulfonylaminocarbonyl-1-oxo-1,3-dihydroisoindol-2-yl)biphenyl-4-carboxylic Acid

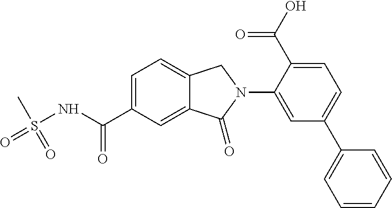 [SMILES: CS(=O)(=O)NC(=O)c1ccc2c(c1)C(=O)N(c1cc(-c3ccccc3)ccc1C(=O)O)C2]

231. 3-(6-Methanesulfonylaminocarbonyl-1-oxo-1,3-dihydroisoindol-2-yl)biphenyl-4-carboxylic acid was prepared as described in synthetic procedure S from 3-(6-methanesulfonylaminocarbonyl-1-oxo-1,3-dihydroisoindol-2-yl)biphenyl-4-carboxylic acid methyl ester (see Example 103); MS m/z: (M+H)⁺ calculated for C₂₃H₁₈N₂O₆S: 451.47. found 451.00. LC/MS retention time: 2.49 minutes.

### Example 105: 3′,4′-Difluoro-3-(6-methanesulfonylaminocarbonyl-1-oxo-1,3-dihydroisoindol-2-yl)biphenyl-4-carboxylic acid methyl ester

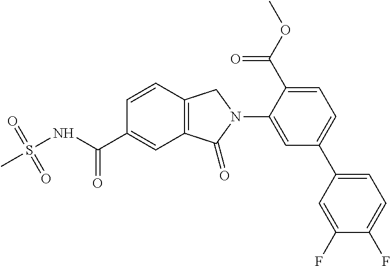 [SMILES: COC(=O)c1ccc(-c2ccc(F)c(F)c2)cc1N1Cc2ccc(C(=O)NS(C)(=O)=O)cc2C1=O]

232. 3′,4′-Difluoro-3-(6-methanesulfonylaminocarbonyl-1-oxo-1,3-dihydroisoindol-2-yl)biphenyl-4-carboxylic acid methyl ester was prepared as described in synthetic procedure R followed by synthetic procedure S from 3-(6-bromo-1-oxo-1,3-dihydroisoindol-2-yl)-3′,4′-difluorobiphenyl-4-carboxylic acid methyl ester (Intermediate 2) and methanesulfonamide; MS m/z: (M+H)⁺ calculated for C₂₄H₁₈F₂N₂O₆S: 501.48. found 501.10. LC/MS retention time: 2.66 minutes.

### Example 106: 3′,4′-Difluoro-3-(6-methanesulfonylaminocarbonyl-1-oxo-1,3-dihydroisoindol-2-yl)biphenyl-4-carboxylic Acid

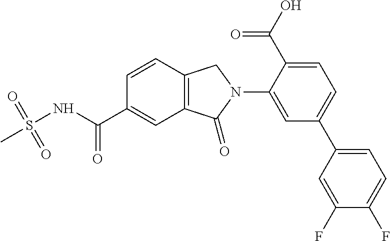 [SMILES: CS(=O)(=O)NC(=O)c1ccc2c(c1)C(=O)N(c1cc(-c3ccc(F)c(F)c3)ccc1C(=O)O)C2]

233. 3′,4′-Difluoro-3-(6-methanesulfonylaminocarbonyl-1-oxo-1,3-dihydroisoindol-2-yl)biphenyl-4-carboxylic acid was prepared as described in synthetic procedure S from 3′,4′-difluoro-3-(6-methanesulfonylaminocarbonyl-1-oxo-1,3-dihydroisoindol-2-yl)biphenyl-4-carboxylic acid methyl ester (see Example 105); MS m/z: (M+H)⁺ calculated for C₂₃H₁₆F₂N₂O₆S: 487.45. found 487.30. LC/MS retention time: 2.47 minutes.

### Example 107: 3′,4′-Difluoro-3-(6-methanesulfonylaminocarbonyl-1-oxo-1,3-dihydroisoindol-2-yl)biphenyl-4-carboxylic acid amide

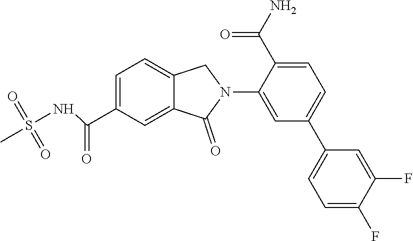 [SMILES: CS(=O)(=O)NC(=O)c1ccc2c(c1)C(=O)N(c1cc(-c3ccc(F)c(F)c3)ccc1C(N)=O)C2]

234. 3′,4′-Difluoro-3-(6-methanesulfonylaminocarbonyl-1-oxo-1,3-dihydroisoindol-2-yl)biphenyl-4-carboxylic acid amide was prepared as described in synthetic procedure P from 3′,4′-difluoro-3-(6-methanesulfonylaminocarbonyl-1-oxo-1,3-dihydroisoindol-2-yl)biphenyl-4-carboxylic acid (see Example 106) and NH₄Cl; MS m/z: (M+H)⁺ calculated for C₂₃H₁₇F₂N₃O₅S: 486.47. found 486.40. LC/MS retention time: 2.33 minutes.

### Example 108: 3′,4′-Difluoro-3-(6-methanesulfonylaminocarbonyl-1-oxo-1,3-dihydroisoindol-2-yl)biphenyl-4-carboxylic acid 2-dimethylamino-ethyl ester

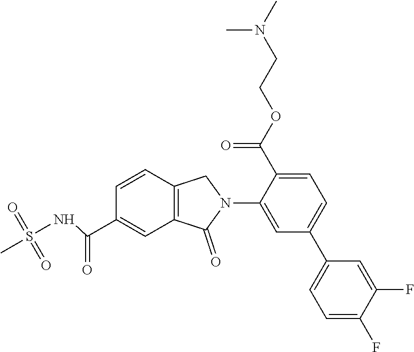 [SMILES: CN(C)CCOC(=O)c1ccc(-c2ccc(F)c(F)c2)cc1N1Cc2ccc(C(=O)NS(C)(=O)=O)cc2C1=O]

235. 3′,4′-Difluoro-3-(6-methanesulfonylaminocarbonyl-1-oxo-1,3-dihydroisoindol-2-yl)biphenyl-4-carboxylic acid 2-dimethylamino-ethyl ester was prepared as described in synthetic procedure P route A from 3′,4′-difluoro-3-(6-methanesulfonylaminocarbonyl-1-oxo-1,3-dihydroisoindol-2-yl)biphenyl-4-carboxylic acid (see Example 106) and 2-dimethylaminoethanol; MS m/z: (M+H)⁺ calculated for C₂₇H₂₅F₂N₃O₆S: 557.58. found 558.10. LC/MS retention time: 2.47 minutes.

### Example 109: 3′,4′-Difluoro-3-(6-methanesulfonylaminocarbonyl-1-oxo-1,3-dihydroisoindol-2-yl)biphenyl-4-carboxylic acid 2-pyrrolidin-1-yl-ethyl ester

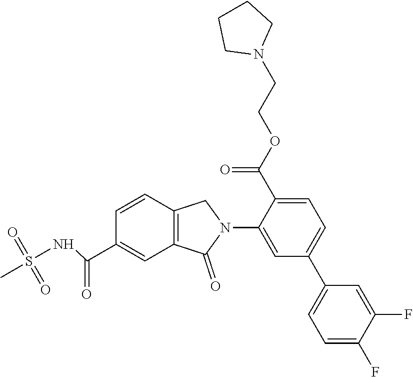 [SMILES: CS(=O)(=O)NC(=O)c1ccc2c(c1)C(=O)N(c1cc(-c3ccc(F)c(F)c3)ccc1C(=O)OCCN1CCCC1)C2]

236. 3′,4′-Difluoro-3-(6-methanesulfonylaminocarbonyl-1-oxo-1,3-dihydroisoindol-2-yl)biphenyl-4-carboxylic acid 2-pyrrolidin-1-yl-ethyl ester was prepared as described in synthetic procedure P route A from 3′,4′-difluoro-3-(6-methanesulfonylaminocarbonyl-1-oxo-1,3-dihydroisoindol-2-yl)biphenyl-4-carboxylic acid (see Example 106) and 1-(2-hydroxyethyl)pyrrolidine; MS m/z: (M+H)⁺ calculated for C₂₉H₂₇F₂N₃O₆S: 584.62. found 584.20. LC/MS retention time: 2.32 minutes.

### Example 110: 3′,4′-Difluoro-3-[1-oxo-6-(1H-[1,2,3]triazol-4-yl)-1,3-dihydroisoindol-2-yl]biphenyl-4-carboxylic acid methyl ester

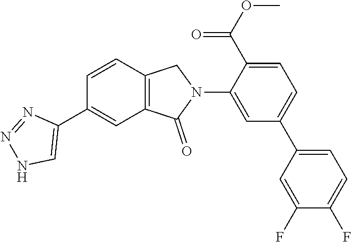 [SMILES: COC(=O)c1ccc(-c2ccc(F)c(F)c2)cc1N1Cc2ccc(-c3c[nH]nn3)cc2C1=O]

237. 3′,4′-Difluoro-3-[1-oxo-6-(1H-[1,2,3]triazol-4-yl)-1,3-dihydroisoindol-2-yl]biphenyl-4-carboxylic acid methyl ester was prepared as described in synthetic procedure Y followed by synthetic procedure Z from 3-(6-bromo-1-oxo-1,3-dihydroisoindol-2-yl)-3′,4′-difluorobiphenyl-4-carboxylic acid methyl ester (Intermediate 2), ethynyltrimethylsilane and sodium azide; MS m/z: (M+H)⁺ calculated for C₂₄H₁₆F₂N₄O₃: 447.42. found 447.10. LC/MS retention time: 2.70 minutes.

### Example 111: 3′,4′-Difluoro-3-[1-oxo-6-(1H-[1,2,3]triazol-4-yl)-1,3-dihydroisoindol-2-yl]biphenyl-4-carboxylic Acid

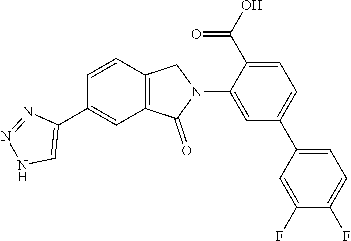 [SMILES: O=C(O)c1ccc(-c2ccc(F)c(F)c2)cc1N1Cc2ccc(-c3c[nH]nn3)cc2C1=O]

238. 3′,4′-Difluoro-3-[1-oxo-6-(1H-[1,2,3]triazol-4-yl)-1,3-dihydroisoindol-2-yl]biphenyl-4-carboxylic acid was prepared as described in in synthetic procedure Z from 3′,4′-difluoro-3-[1-oxo-6-(1H-[1,2,3]triazol-4-yl)-1,3-dihydroisoindol-2-yl]biphenyl-4-carboxylic acid methyl ester (see Example 110); MS m/z: (M+H)⁺ calculated for C₂₃H₁₄F₂N₄O₃: 433.39. found 433.30. LC/MS retention time: 2.44 minutes.

### Example 112: 3′,4′-Difluoro-3-[1-oxo-6-(1H-[1,2,3]triazol-4-yl)-1,3-dihydroisoindol-2-yl]biphenyl-4-carboxylic acid 2-dimethylamino-ethyl ester

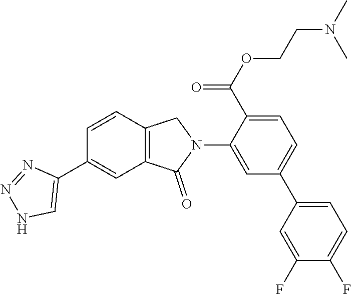 [SMILES: CN(C)CCOC(=O)c1ccc(-c2ccc(F)c(F)c2)cc1N1Cc2ccc(-c3c[nH]nn3)cc2C1=O]

239. 3′,4′-Difluoro-3-[1-oxo-6-(1H-[1,2,3]triazol-4-yl)-1,3-dihydroisoindol-2-yl]biphenyl-4-carboxylic acid 2-dimethylamino-ethyl ester was prepared as described in synthetic procedure P route A (reaction time 2.5 h, room temperature) from 3′,4′-difluoro-3-[1-oxo-6-(1H-[1,2,3]triazol-4-yl)-1,3-dihydroisoindol-2-yl]biphenyl-4-carboxylic acid (see Example 111) and 2-dimethylaminoethanol; MS m/z: (M+H)⁺ calculated for C₂₇H₂₃F₂N₅O₃: 504.51. found 504.10. LC/MS retention time: 2.31 minutes.

### Example 112a: 3′,4′-Difluoro-3-[1-oxo-6-(1H-[1,2,3]triazol-4-yl)-1,3-dihydroisoindol-2-yl]biphenyl-4-carboxylic acid (2-oxo-1,3-oxazolidin-5-yl)methyl ester

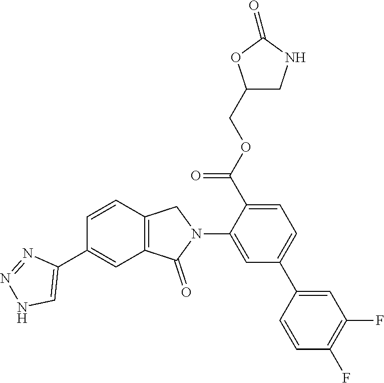 [SMILES: O=C1NCC(COC(=O)c2ccc(-c3ccc(F)c(F)c3)cc2N2Cc3ccc(-c4c[nH]nn4)cc3C2=O)O1]

240. 3′,4′-Difluoro-3-[1-oxo-6-(1H-[1,2,3]triazol-4-yl)-1,3-dihydroisoindol-2-yl]biphenyl-4-carboxylic acid (2-oxo-1,3-oxazolidin-5-yl)methyl ester ester was prepared as described in synthetic procedure P route F from 3′,4′-difluoro-3-[1-oxo-6-(1H-[1,2,3]triazol-4-yl)-1,3-dihydroisoindol-2-yl]biphenyl-4-carboxylic acid (see Example 111) and 5-(hydroxymethyl)-1,3-oxazolidin-2-one; MS m/z: (M+H)⁺ calculated for C₂₇H₁₉F₂N₅O₅: 530.14. found 530.31. LC/MS retention time: 1.38 minutes.

### Example 112b: 3′,4′-Difluoro-3-[1-oxo-6-(1H-[1,2,3]triazol-4-yl)-1,3-dihydroisoindol-2-yl]biphenyl-4-carboxylic acid 2,3-dihydroxypropyl ester

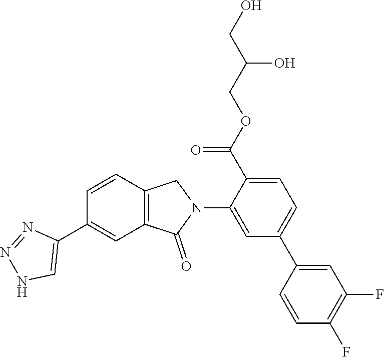 [SMILES: O=C(OCC(O)CO)c1ccc(-c2ccc(F)c(F)c2)cc1N1Cc2ccc(-c3c[nH]nn3)cc2C1=O]

241. 3′,4′-Difluoro-3-[1-oxo-6-(1H-[1,2,3]triazol-4-yl)-1,3-dihydroisoindol-2-yl]biphenyl-4-carboxylic acid 2,3-dihydroxypropyl ester was prepared as described in synthetic procedure P route B from 3′,4′-difluoro-3-[1-oxo-6-(1H-[1,2,3]triazol-4-yl)-1,3-dihydroisoindol-2-yl]biphenyl-4-carboxylic acid (see Example 111) and glycerol; MS m/z: (M+H)⁺ calculated for C₂₆H₂₀F₂N₄O₅: 507.15. found 507.31. LC/MS retention time: 1.62 minutes.

### Example 112c: 3′,4′-Difluoro-3-[1-oxo-6-(1H-[1,2,3]triazol-4-yl)-1,3-dihydroisoindol-2-yl]biphenyl-4-carboxylic acid butan-2-yl ester

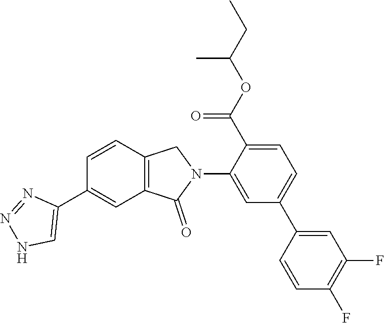 [SMILES: CCC(C)OC(=O)c1ccc(-c2ccc(F)c(F)c2)cc1N1Cc2ccc(-c3c[nH]nn3)cc2C1=O]

242. 3′,4′-Difluoro-3-[1-oxo-6-(1H-[1,2,3]triazol-4-yl)-1,3-dihydroisoindol-2-yl]biphenyl-4-carboxylic acid butan-2-yl ester was prepared as described in synthetic procedure P route B from 3′,4′-difluoro-3-[1-oxo-6-(1H-[1,2,3]triazol-4-yl)-1,3-dihydroisoindol-2-yl]biphenyl-4-carboxylic acid (see Example 111) and butan-2-ol; MS m/z: (M+H)⁺ calculated for C₂₇H₂₂F₂N₄O₃: 489.17. found 489.34. LC/MS retention time: 1.27 minutes.

### Example 112d: 3′,4′-Difluoro-3-[1-oxo-6-(1H-[1,2,3]triazol-4-yl)-1,3-dihydroisoindol-2-yl]biphenyl-4-carboxylic acid 1-(dimethylamino)propan-2-yl ester

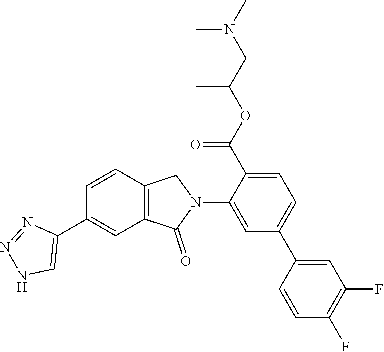 [SMILES: CC(CN(C)C)OC(=O)c1ccc(-c2ccc(F)c(F)c2)cc1N1Cc2ccc(-c3c[nH]nn3)cc2C1=O]

243. 3′,4′-Difluoro-3-[1-oxo-6-(1H-[1,2,3]triazol-4-yl)-1,3-dihydroisoindol-2-yl]biphenyl-4-carboxylic acid 1-(dimethylamino)propan-2-yl ester was prepared as described in synthetic procedure P route A (reaction time 7.5 h, room temperature) from 3′,4′-difluoro-3-[1-oxo-6-(1H-[1,2,3]triazol-4-yl)-1,3-dihydroisoindol-2-yl]biphenyl-4-carboxylic acid (see Example 111) and 1-(dimethylamino)propan-2-ol; MS m/z: (M+H)⁺ calculated for C₂₈H₂₅F₂N₅O₃: 518.21. found 518.37. LC/MS retention time: 1.52 minutes.

### Example 113: 3′,4′-Difluoro-3-[1-oxo-6-(1H-[1,2,3]triazol-4-yl)-1,3-dihydroisoindol-2-yl]biphenyl-4-carboxylic acid amide

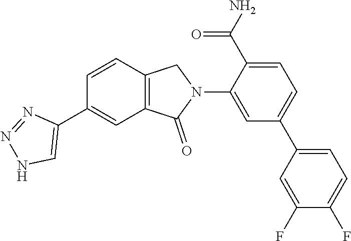 [SMILES: NC(=O)c1ccc(-c2ccc(F)c(F)c2)cc1N1Cc2ccc(-c3c[nH]nn3)cc2C1=O]

244. 3′,4′-Difluoro-3-[1-oxo-6-(1H-[1,2,3]triazol-4-yl)-1,3-dihydroisoindol-2-yl]biphenyl-4-carboxylic acid amide was prepared as described in synthetic procedure P from 3′,4′-difluoro-3-[1-oxo-6-(1H-[1,2,3]triazol-4-yl)-1,3-dihydroisoindol-2-yl]biphenyl-4-carboxylic acid (see Example 111) and NH₄Cl; MS m/z: (M+H)⁺ calculated for C₂₃H₁₅F₂N₅O₂: 431.41. found 432.40. LC/MS retention time: 2.33 minutes.

### Example 114: 3′,4′-Difluoro-3-[1-oxo-6-(1H-[1,2,3]triazol-4-yl)-1,3-dihydroisoindol-2-yl]biphenyl-4-carboxylic acid 2-methoxy-ethyl ester

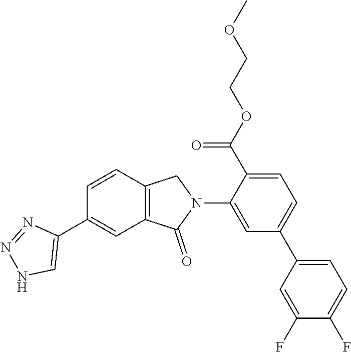 [SMILES: COCCOC(=O)c1ccc(-c2ccc(F)c(F)c2)cc1N1Cc2ccc(-c3c[nH]nn3)cc2C1=O]

245. 3′,4′-Difluoro-3-[1-oxo-6-(1H-[1,2,3]triazol-4-yl)-1,3-dihydroisoindol-2-yl]biphenyl-4-carboxylic acid 2-methoxy-ethyl ester was prepared as described in synthetic procedure P route A from 3′,4′-difluoro-3-[1-oxo-6-(1H-[1,2,3]triazol-4-yl)-1,3-dihydroisoindol-2-yl]biphenyl-4-carboxylic acid (see Example 111) and 2-methoxyethanol; MS m/z: (M+H)⁺ calculated for C₂₆H₂₀F₂N₄O₄: 491.47. found 491.20. LC/MS retention time: 2.69 minutes.

### Example 115: 3′,4′-Difluoro-3-[1-oxo-6-(1H-[1,2,3]triazol-4-yl)-1,3-dihydroisoindol-2-yl]biphenyl-4-carboxylic acid 2-pyrrolidin-1-yl-ethyl ester

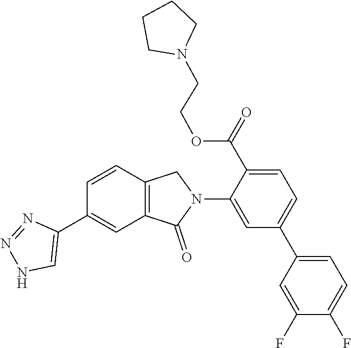 [SMILES: O=C(OCCN1CCCC1)c1ccc(-c2ccc(F)c(F)c2)cc1N1Cc2ccc(-c3c[nH]nn3)cc2C1=O]

246. 3′,4′-Difluoro-3-[1-oxo-6-(1H-[1,2,3]triazol-4-yl)-1,3-dihydroisoindol-2-yl]biphenyl-4-carboxylic acid 2-pyrrolidin-1-yl-ethyl ester was prepared as described in synthetic procedure P route A (reaction time 1.5 h, room temperature) from 3′,4′-difluoro-3-[1-oxo-6-(1H-[1,2,3]triazol-4-yl)-1,3-dihydroisoindol-2-yl]biphenyl-4-carboxylic acid (see Example 111) and 1-(2-hydroxyethyl)pyrrolidine; MS m/z: (M+H)⁺ calculated for C₂₉H₂₅F₂N₃O₃: 530.55. found 530.20. LC/MS retention time: 2.40 minutes.

### Example 116: 3′,4′-Difluoro-3-[1-oxo-6-(1H-[1,2,3]triazol-4-yl)-1,3-dihydro-isoindol-2-yl]-biphenyl-4-carboxylic acid (5-methyl-2-oxo-[1,3]dioxol-4-yl)methyl ester

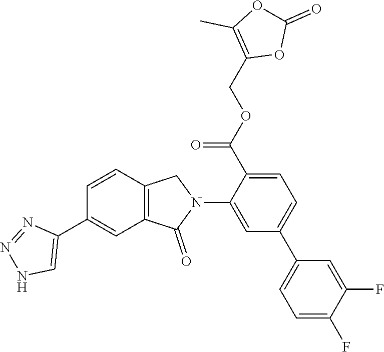 [SMILES: Cc1oc(=O)oc1COC(=O)c1ccc(-c2ccc(F)c(F)c2)cc1N1Cc2ccc(-c3c[nH]nn3)cc2C1=O]

247. 3′,4′-Difluoro-3-[1-oxo-6-(1H-[1,2,3]triazol-4-yl)-1,3-dihydro-isoindol-2-yl]-biphenyl-4-carboxylic acid (5-methyl-2-oxo-[1,3]dioxol-4-yl)methyl ester was prepared as described in synthetic procedure P route C from 3′,4′-difluoro-3-[1-oxo-6-(1H-[1,2,3]triazol-4-yl)-1,3-dihydroisoindol-2-yl]biphenyl-4-carboxylic acid (see Example 111) and 4-(hydroxymethyl)-5-methyl-1,3-dioxol-2-one; MS m/z: (M+H)⁺ calculated for C₂₈H₁₈F₂N₄O₆: 545.48. found 545.58. LC/MS retention time: 2.58 minutes.

### Example 117: 3′,4′-Difluoro-3-[1-oxo-6-(1H-[1,2,3]triazol-4-yl)-1,3-dihydro-isoindol-2-yl]-biphenyl-4-carboxylic acid tert-butyl ester

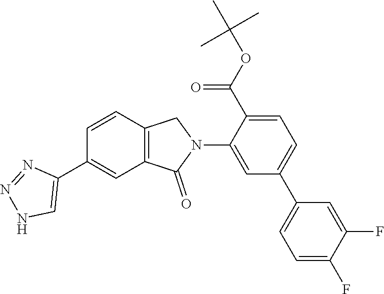 [SMILES: CC(C)(C)OC(=O)c1ccc(-c2ccc(F)c(F)c2)cc1N1Cc2ccc(-c3c[nH]nn3)cc2C1=O]

248. 3′,4′-Difluoro-3-[1-oxo-6-(1H-[1,2,3]triazol-4-yl)-1,3-dihydro-isoindol-2-yl]-biphenyl-4-carboxylic acid tert-butyl ester was prepared as described in synthetic procedure P route B from 3′,4′-difluoro-3-[1-oxo-6-(1H-[1,2,3]triazol-4-yl)-1,3-dihydroisoindol-2-yl]biphenyl-4-carboxylic acid (see Example 111) and tert-butanol; MS m/z: (M+H)⁺ calculated for C₂₇H₂₂F₂N₄O₃: 489.50. found 489.41. LC/MS retention time: 2.51 minutes.

### Example 118: 3′,4′-Difluoro-3-[1-oxo-6-(1H-[1,2,3]triazol-4-yl)-1,3-dihydro-isoindol-2-yl]-biphenyl-4-carboxylic acid isopropyl ester

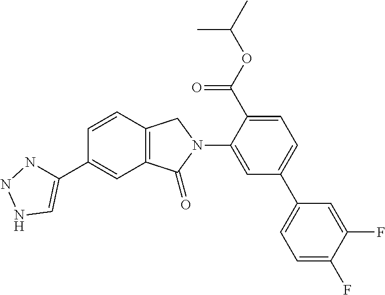 [SMILES: CC(C)OC(=O)c1ccc(-c2ccc(F)c(F)c2)cc1N1Cc2ccc(C3=CNNN3)cc2C1=O]

249. 3′,4′-Difluoro-3-[1-oxo-6-(1H-[1,2,3]triazol-4-yl)-1,3-dihydro-isoindol-2-yl]-biphenyl-4-carboxylic acid isopropyl ester was prepared as described in synthetic procedure P route B from 3′,4′-difluoro-3-[1-oxo-6-(1H-[1,2,3]triazol-4-yl)-1,3-dihydroisoindol-2-yl]biphenyl-4-carboxylic acid (see Example 111) and isopropanol; MS m/z: (M+H)⁺ calculated for C₂₆H₂₀F₂N₄O₃: 475.47. found 475.29. LC/MS retention time: 2.45 minutes.

### Example 119: 2-(4-Carbamoyl-3′,4′-difluorobiphenyl-3-yl)-3-oxo-2,3-dihydro-1H-isoindole-5-carboxylic acid

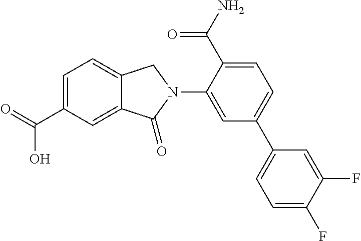 [SMILES: NC(=O)c1ccc(-c2ccc(F)c(F)c2)cc1N1Cc2ccc(C(=O)O)cc2C1=O]

250. 2-(4-Carbamoyl-3′,4′-difluorobiphenyl-3-yl)-3-oxo-2,3-dihydro-1H-isoindole-5-carboxylic acid was prepared as described in synthetic procedure P followed by synthetic procedure R from crude 3-(6-bromo-1-oxo-1,3-dihydroisoindol-2-yl)-3′,4′-difluorobiphenyl-4-carboxylic acid (Intermediate 2a) and NH₄Cl; MS m/z: (M+H)⁺ calculated for C₂₂H₁₄F₂N₂O₄: 409.36. found 409.30. LC/MS retention time: 2.33 minutes.

### Example 120: 2-[4-(2-Dimethylamino-ethoxycarbonyl)-3′,4′-difluorobiphenyl-3-yl]-3-oxo-2,3-dihydro-1H-isoindole-5-carboxylic Acid

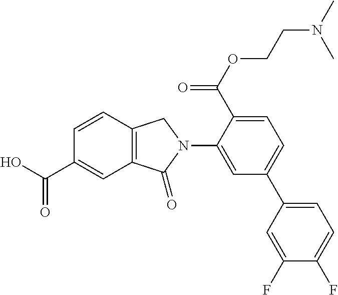 [SMILES: CN(C)CCOC(=O)c1ccc(-c2ccc(F)c(F)c2)cc1N1Cc2ccc(C(=O)O)cc2C1=O]

251. 2-[4-(2-Dimethylamino-ethoxycarbonyl)-3′,4′-difluorobiphenyl-3-yl]-3-oxo-2,3-dihydro-1H-isoindole-5-carboxylic acid was prepared as described in synthetic procedure P route A followed by synthetic procedure R from crude 3-(6-bromo-1-oxo-1,3-dihydroisoindol-2-yl)-3′,4′-difluorobiphenyl-4-carboxylic acid (Intermediate 2a) and 2-dimethylaminoethanol; MS m/z: (M+H)⁺ calculated for C₂₆H₂₂F₂N₂O₅: 481.47. found 481.30. LC/MS retention time: 2.29 minutes.

### Example 121: 3′,4′-Difluoro-3-(1-oxo-6-tetrazol-1-yl-1,3-dihydroisoindol-2-yl)biphenyl-4-carboxylic acid methyl ester

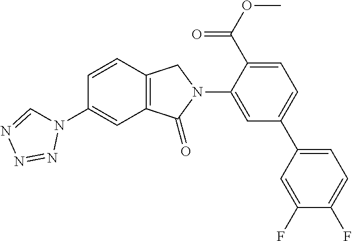 [SMILES: COC(=O)c1ccc(-c2ccc(F)c(F)c2)cc1N1Cc2ccc(-n3cnnn3)cc2C1=O]

252. 3′,4′-Difluoro-3-(1-oxo-6-tetrazol-1-yl-1,3-dihydroisoindol-2-yl)biphenyl-4-carboxylic acid methyl ester was prepared as described in synthetic procedure X from 3-(6-amino-1-oxo-1,3-dihydroisoindol-2-yl)-3′,4′-difluorobiphenyl-4-carboxylic acid methyl ester (Intermediate 4), sodium azide and trimethyl orthoformate; MS m/z: (M+H)⁺ calculated for C₂₃H₁₅F₂N₅O₃: 448.40. found 448.00. LC/MS retention time: 2.59 minutes.

### Example 122: 3′,4′-Difluoro-3-(1-oxo-6-tetrazol-1-yl-1,3-dihydroisoindol-2-yl)biphenyl-4-carboxylic Acid

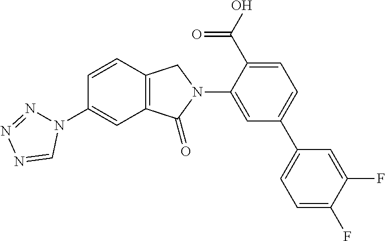 [SMILES: O=C(O)c1ccc(-c2ccc(F)c(F)c2)cc1N1Cc2ccc(-n3cnnn3)cc2C1=O]

253. 3′,4′-Difluoro-3-(1-oxo-6-tetrazol-1-yl-1,3-dihydroisoindol-2-yl)biphenyl-4-carboxylic acid was prepared as described in synthetic procedure X from 3′,4′-difluoro-3-(1-oxo-6-tetrazol-1-yl-1,3-dihydroisoindol-2-yl)biphenyl-4-carboxylic acid methyl ester (see Example 121); MS m/z: (M+H)⁺ calculated for C₂₂H₁₃F₂N₅O₃: 434.38. found 433.90. LC/MS retention time: 2.15 minutes.

### Example 123: 2-(4-Carbamoyl-3′,4′-difluorobiphenyl-3-yl)-3-oxo-2,3-dihydro-1H-isoindole-5-carboxylic acid butyl ester

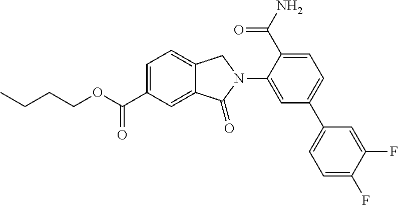 [SMILES: CCCCOC(=O)c1ccc2c(c1)C(=O)N(c1cc(-c3ccc(F)c(F)c3)ccc1C(N)=O)C2]

254. 2-(4-Carbamoyl-3′,4′-difluorobiphenyl-3-yl)-3-oxo-2,3-dihydro-1H-isoindole-5-carboxylic acid butyl ester was prepared as described in synthetic procedure AD from 2-(4-carbamoyl-3′,4′-difluorobiphenyl-3-yl)-3-oxo-2,3-dihydro-1H-isoindole-5-carboxylic acid (see Example 119) and n-butanol; MS m/z: (M+H)⁺ calculated for C₂₆H₂₂F₂N₂O₄: 465.47. found 465.40. LC/MS retention time: 2.90 minutes.

### Example 124: 3′,4′-Difluoro-3-(6-methanesulfonylaminocarbonyl-1-oxo-1,3-dihydro-isoindol-2-yl)-biphenyl-4-carboxylic acid 3-dimethylamino-propyl ester

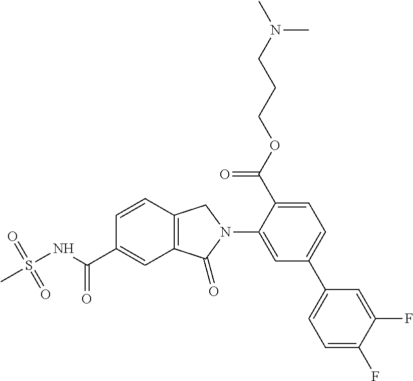 [SMILES: CN(C)CCCOC(=O)c1ccc(-c2ccc(F)c(F)c2)cc1N1Cc2ccc(C(=O)NS(C)(=O)=O)cc2C1=O]

255. 3′,4′-Difluoro-3-(6-methanesulfonylaminocarbonyl-1-oxo-1,3-dihydro-isoindol-2-yl)-biphenyl-4-carboxylic acid 3-dimethylamino-propyl ester was prepared as described in synthetic procedure P route A from 3′,4′-difluoro-3-(6-methanesulfonylaminocarbonyl-1-oxo-1,3-dihydroisoindol-2-yl)biphenyl-4-carboxylic acid (see Example 106) and 3-dimethylamino-1-propanol; MS m/z: (M+H)⁺ calculated for C₂₈H₂₇F₂N₂O₆S: 572.60. found 572.26. LC/MS retention time: 2.30 minutes.

### Example 125: 3′,4′-Difluoro-3-(6-methanesulfonylaminocarbonyl-1-oxo-1,3-dihydro-isoindol-2-yl)-biphenyl-4-carboxylic acid isopropyl ester

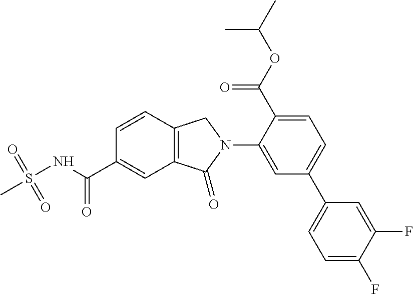 [SMILES: CC(C)OC(=O)c1ccc(-c2ccc(F)c(F)c2)cc1N1Cc2ccc(C(=O)NS(C)(=O)=O)cc2C1=O]

256. 3′,4′-Difluoro-3-(6-methanesulfonylaminocarbonyl-1-oxo-1,3-dihydro-isoindol-2-yl)-biphenyl-4-carboxylic acid isopropyl ester was prepared as described in synthetic procedure P route A from 3,4-difluoro-3-(6-methanesulfonylaminocarbonyl-1-oxo-1,3-dihydroisoindol-2-yl)biphenyl-4-carboxylic acid (see Example 106) and isopropanol; MS m/z: (M+H)⁺ calculated for C₂₆H₂₂F₂N₂O₆S: 529.54. found 528.71. LC/MS retention time: 2.85 minutes.

### Example 126: 2-(4-Carboxy-3′,4′-difluoro-biphenyl-3-yl)-3-oxo-2,3-dihydro-1H-isoindole-5-carboxylic acid

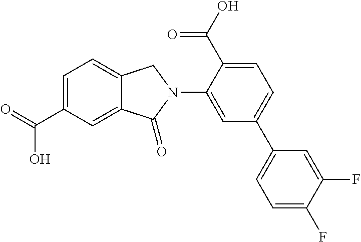 [SMILES: O=C(O)c1ccc2c(c1)C(=O)N(c1cc(-c3ccc(F)c(F)c3)ccc1C(=O)O)C2]

257. 2-(4-Carboxy-3′,4′-difluoro-biphenyl-3-yl)-3-oxo-2,3-dihydro-1H-isoindole-5-carboxylic acid was prepared as described in synthetic procedure R from crude 3-(6-bromo-1-oxo-1,3-dihydroisoindol-2-yl)-3′,4′-difluorobiphenyl-4-carboxylic acid (Intermediate 2a); MS m/z: (M+H)⁺ calculated for C₂₂H₁₃F₂NO₅: 410.35. found 410.51. LC/MS retention time: 2.10 minutes.

### Example 127: 2-(3′,4′-Difluoro-4-methoxycarbonylbiphenyl-3-yl)-3-oxo-2,3-dihydro-1H-isoindole-5-carboxylic acid methyl ester

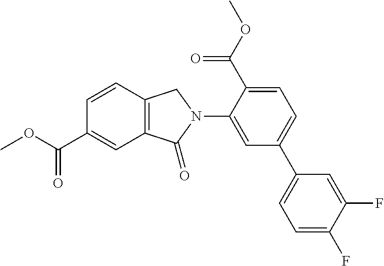 [SMILES: COC(=O)c1ccc2c(c1)C(=O)N(c1cc(-c3ccc(F)c(F)c3)ccc1C(=O)OC)C2]

258. 2-(3′,4′-Difluoro-4-methoxycarbonylbiphenyl-3-yl)-3-oxo-2,3-dihydro-1H-isoindole-5-carboxylic acid methyl ester was prepared as described in synthetic procedure R followed by synthetic procedure AD from 3-(6-bromo-1-oxo-1,3-dihydroisoindol-2-yl)-3′,4′-difluorobiphenyl-4-carboxylic acid methyl ester (Intermediate 2) and methanol; MS m/z: (M+H)⁺ calculated for C₂₄H₁₇F₂NOs: 410.35. found 410.51. LC/MS retention time: 2.52 minutes.

### Example 128: 2-(4-Carboxy-3′,4′-difluoro-biphenyl-3-yl)-3-oxo-2,3-dihydro-1H-isoindole-5-carboxylic acid butyl ester

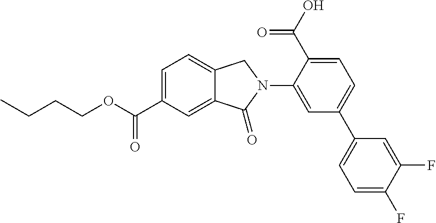 [SMILES: CCCCOC(=O)c1ccc2c(c1)C(=O)N(c1cc(-c3ccc(F)c(F)c3)ccc1C(=O)O)C2]

259. 2-(4-Carboxy-3′,4′-difluoro-biphenyl-3-yl)-3-oxo-2,3-dihydro-1H-isoindole-5-carboxylic acid butyl ester was prepared as described in synthetic procedure AE from 2-{4-[(benzyloxy)carbonyl]-3′,4′-difluoro[1,1′-biphenyl]-3-yl}-3-oxo-2,3-dihydro-1H-isoindole-5-carboxylic acid (Intermediate 3b) and n-butanol; MS m/z: (M+H)⁺ calculated for C₂₆H₂₁F₂NO₅: 466.46. found 466.29. LC/MS retention time: 2.34 minutes.

### Example 129: 2-(4-Carboxy-3′,4′-difluorobiphenyl-3-yl)-3-oxo-2,3-dihydro-1H-isoindole-5-carboxylic acid 2-dimethylamino-ethyl ester

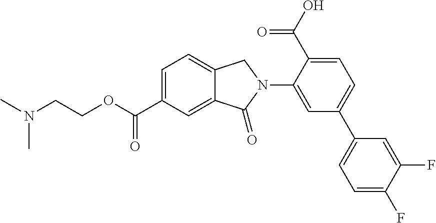 [SMILES: CN(C)CCOC(=O)c1ccc2c(c1)C(=O)N(c1cc(-c3ccc(F)c(F)c3)ccc1C(=O)O)C2]

260. 2-(4-Carboxy-3′,4′-difluorobiphenyl-3-yl)-3-oxo-2,3-dihydro-1H-isoindole-5-carboxylic acid 2-dimethylamino-ethyl ester was prepared as described in synthetic procedure AE from 2-{4-[(benzyloxy)carbonyl]-3′,4′-difluoro[1,1′-biphenyl]-3-yl}-3-oxo-2,3-dihydro-1H-isoindole-5-carboxylic acid (Intermediate 3b) and 2-dimethylaminoethanol; MS m/z: (M+H)⁺ calculated for C₂₆H₂₂F₂N₂O₅: 481.47. found 481.29. LC/MS retention time: 2.22 minutes.

### Example 130: 3-(6-Acetylamino-1-oxo-1,3-dihydroisoindol-2-yl)-3′,4′-difluoro-biphenyl-4-carboxylic Acid

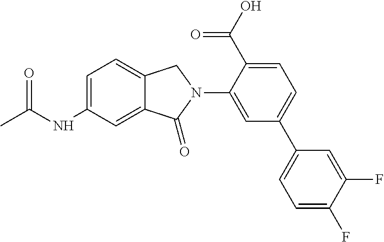 [SMILES: CC(=O)Nc1ccc2c(c1)C(=O)N(c1cc(-c3ccc(F)c(F)c3)ccc1C(=O)O)C2]

261. 3-(6-Acetylamino-1-oxo-1,3-dihydroisoindol-2-yl)-3′,4′-difluoro-biphenyl-4-carboxylic acid was prepared as described in synthetic procedure V from 3-(6-amino-1-oxo-1,3-dihydroisoindol-2-yl)-3′,4′-difluorobiphenyl-4-carboxylic acid methyl ester (Intermediate 4) and acetic anhydride; MS m/z: (M+H)⁺ calculated for C₂₃H₁₅F₂N₂O₄: 423.10. found 423.39. LC/MS retention time: 2.01 minutes.

### Example 131: 3′,4′-Difluoro-3-(6-methanesulfonylamino-1-oxo-1,3-dihydro-isoindol-2-yl)-biphenyl-4-carboxylic Acid

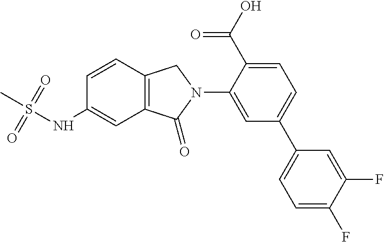 [SMILES: CS(=O)(=O)Nc1ccc2c(c1)C(=O)N(c1cc(-c3ccc(F)c(F)c3)ccc1C(=O)O)C2]

262. 3′,4′-Difluoro-3-(6-methanesulfonylamino-1-oxo-1,3-dihydro-isoindol-2-yl)-biphenyl-4-carboxylic acid was prepared as described in synthetic procedure W from 3-(6-amino-1-oxo-1,3-dihydroisoindol-2-yl)-3′,4′-difluorobiphenyl-4-carboxylic acid methyl ester (Intermediate 4) and methanesulfonyl chloride; MS m/z: (M+H)⁺ calculated for C₂₂H₁₆F₂N₂O₅S: 459.10. found 459.49. LC/MS retention time: 2.02 minutes.

### Example 132: 3′,4′-Difluoro-3-[1-oxo-6-(toluene-4-sulfonylamino)-1,3-dihydro-isoindol-2-yl]-biphenyl-4-carboxylic acid

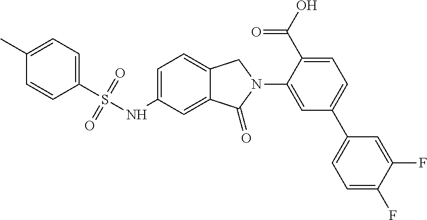 [SMILES: Cc1ccc(S(=O)(=O)Nc2ccc3c(c2)C(=O)N(c2cc(-c4ccc(F)c(F)c4)ccc2C(=O)O)C3)cc1]

263. 3′,4′-Difluoro-3-[1-oxo-6-(toluene-4-sulfonylamino)-1,3-dihydro-isoindol-2-yl]-biphenyl-4-carboxylic acid was prepared as described in synthetic procedure W from 3-(6-amino-1-oxo-1,3-dihydroisoindol-2-yl)-3′,4′-difluorobiphenyl-4-carboxylic acid methyl ester (Intermediate 4) and 4-toluenesulfonyl chloride; MS m/z: (M+H)⁺ calculated for C₂₈H₂₀F₂N₂O₅S: 535.54. found 534.54. LC/MS retention time: 2.44 minutes.

### Example 133: 2-(3-Cyano-4,5-diphenylthiophen-2-yl)-1,3-dioxo-2,3-dihydro-1H-isoindole-5-carboxylic acid

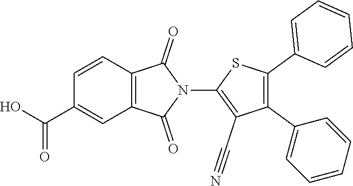 [SMILES: N#Cc1c(N2C(=O)c3ccc(C(=O)O)cc3C2=O)sc(-c2ccccc2)c1-c1ccccc1]

264. 2-(3-Cyano-4,5-diphenylthiophen-2-yl)-1,3-dioxo-2,3-dihydro-1H-isoindole-5-carboxylic acid was prepared as described in synthetic procedure K from trimellitic anhydride and 2-amino-4,5-diphenylthiophene-3-carbonitrile (Intermediate 7a); (M+H)⁺ calculated for C₂₆H₁₄N₂O₄S: 451.48. found 450.98. LC/MS retention time: 2.94 minutes.

### Example 134: 2-(3-Carboxybiphenyl-4-yl)-1,3-dioxo-2,3-dihydro-1H-isoindole-5-carboxylic acid

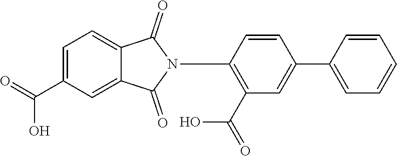 [SMILES: O=C(O)c1ccc2c(c1)C(=O)N(c1ccc(-c3ccccc3)cc1C(=O)O)C2=O]

265. 2-(3-Carboxybiphenyl-4-yl)-1,3-dioxo-2,3-dihydro-1H-isoindole-5-carboxylic acid was prepared as described in synthetic procedure K from trimellitic anhydride and 4-amino[1,1′-biphenyl]-3-carboxylic acid (Intermediate 13); MS m/z: (M+H)⁺ calculated for C₂₂H₁₃NO₆: 388.35. found 388.45; LC/MS retention time: 2.63 minutes.

### Example 135: N-(Benzenesulfonyl)-2-(3-(1H-tetrazol-5-yl)-[1,1′-biphenyl]-4-yl)-1,3-dioxo-2,3-dihydro-1H-isoindole-5-carboxamide

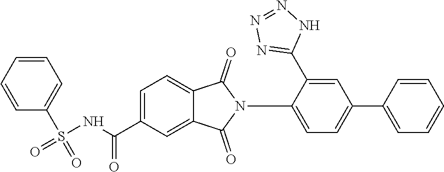 [SMILES: O=C(NS(=O)(=O)c1ccccc1)c1ccc2c(c1)C(=O)N(c1ccc(-c3ccccc3)cc1-c1nnn[nH]1)C2=O]

266. N-(Benzenesulfonyl)-2-(3-(1H-tetrazol-5-yl)-[1,1′-biphenyl]-4-yl)-1,3-dioxo-2,3-dihydro-1H-isoindole-5-carboxamide was prepared as described in synthetic procedure K2 from crude N-(benzenesulfonyl)-1,3-dioxo-1,3-dihydro-2-benzofuran-5-carboxamide (Intermediate 11c) and crude 3-(1H-tetrazol-5-yl)[1,1′-biphenyl]-4-amine (Intermediate 18); MS m/z: (M+H)⁺ calculated for C₂₈H₁₈N₆O₅S: 550.54. found 550.85. LC/MS retention time: 2.20 minutes.

### Example 136: (2S)-2-({2-[3-Cyano-4-(4-methoxyphenyl)-5-methyl-thiophen-2-yl]-1,3-dioxo-2,3-dihydro-1H-isoindole-5-carbonyl}-amino)-3-(4-hydroxyphenyl)propanoic acid

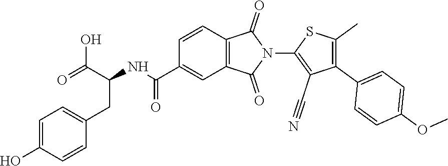 [SMILES: COc1ccc(-c2c(C)sc(N3C(=O)c4ccc(C(=O)N[C@@H](Cc5ccc(O)cc5)C(=O)O)cc4C3=O)c2C#N)cc1]

267. (2S)-2-({2-[3-Cyano-4-(4-methoxyphenyl)-5-methyl-thiophen-2-yl]-1,3-dioxo-2,3-dihydro-1H-isoindole-5-carbonyl}-amino)-3-(4-hydroxyphenyl)propanoic acid was prepared as described in synthetic procedure AA from 2-[3-cyano-4-(4-methoxyphenyl)-5-methylthiophen-2-yl]-1,3-dioxo-2,3-dihydro-1H-isoindole-5-carboxylic acid (see Example 6) and N-Fmoc-L-tyrosine; MS m/z: (M+H)⁺ calculated for C₃₂H₂₅N₃O₇S: 582.61. found 581.85. LC/MS retention time: 2.23 minutes.

### Example 137: (2S)-2-({2-[3-Cyano-4-(4-methoxyphenyl)-5-methyl-thiophen-2-yl]-1,3-dioxo-2,3-dihydro-1H-isoindole-5-carbonyl}-amino)-3-phenylpropanoic acid

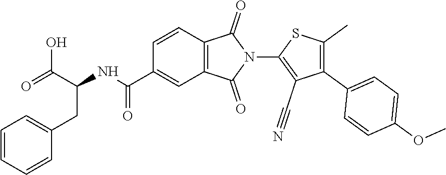 [SMILES: COc1ccc(-c2c(C)sc(N3C(=O)c4ccc(C(=O)N[C@@H](Cc5ccccc5)C(=O)O)cc4C3=O)c2C#N)cc1]

268. (2S)-2-({2-[3-Cyano-4-(4-methoxyphenyl)-5-methyl-thiophen-2-yl]-1,3-dioxo-2,3-dihydro-1H-isoindole-5-carbonyl}-amino)-3-phenylpropanoic acid was prepared as described in synthetic procedure AA from 2-[3-cyano-4-(4-methoxyphenyl)-5-methylthiophen-2-yl]-1,3-dioxo-2,3-dihydro-1H-isoindole-5-carboxylic acid (see Example 6) and Fmoc-L-phenylalanine; MS m/z: (M+H)⁺ calculated for C₃2H₂₅N₃O₆S: 566.61. found 566.24. LC/MS retention time: 2.54 minutes.

### Example 138: 2-(4-Carboxy-3′,4′-difluoro[1,1′-biphenyl]-3-yl)-1,3-dioxo-2,3-dihydro-1H-isoindole-5-carboxylic acid

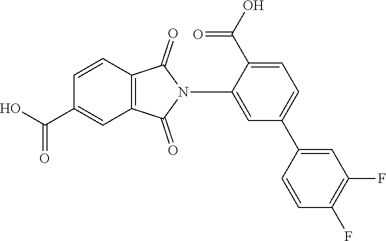 [SMILES: O=C(O)c1ccc2c(c1)C(=O)N(c1cc(-c3ccc(F)c(F)c3)ccc1C(=O)O)C2=O]

269. 2-(4-Carboxy-3′,4′-difluoro[1,1′-biphenyl]-3-yl)-1,3-dioxo-2,3-dihydro-1H-isoindole-5-carboxylic acid was prepared as described in synthetic procedure K from trimellitic anhydride and 3-amino-3′,4′-difluoro[1,1′-biphenyl]-4-carboxylic acid Intermediate 1d) as described in synthetic procedure B); MS m/z: (M+H)⁺ calculated for C₂₂H₁₁F₂NO₆: 424.32. found 423.99. LC/MS retention time: 2.19 minutes.

### Example 139: 2-(4-Carboxy-2′,3′,4′-trifluoro[1,1′-biphenyl]-3-yl)-1,3-dioxo-2,3-dihydro-1H-isoindole-5-carboxylic Acid

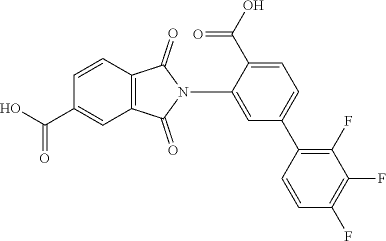 [SMILES: O=C(O)c1ccc2c(c1)C(=O)N(c1cc(-c3ccc(F)c(F)c3F)ccc1C(=O)O)C2=O]

270. 2-(4-Carboxy-2′,3′,4′-trifluoro[1,1′-biphenyl]-3-yl)-1,3-dioxo-2,3-dihydro-1H-isoindole-5-carboxylic acid was prepared as described in synthetic procedure K from trimellitic anhydride and 3-amino-2′,3′,4′-trifluoro[1,1′-biphenyl]-4-carboxylic acid (was obtained as described in synthetic procedure A followed by synthetic procedure B from methyl 2-amino-4-bromobenzoate and 2,3,4-trifluorophenylboronic acid); MS m/z: (M+H)⁺ calculated for C₂₂H₁₀F₃NO₆: 442.31. found 442.28. LC/MS retention time: 2.16 minutes.

### Example 140: 2-(4-Carboxy-2′,4′,5′-trifluoro[1,1′-biphenyl]-3-yl)-1,3-dioxo-2,3-dihydro-1H-isoindole-5-carboxylic Acid

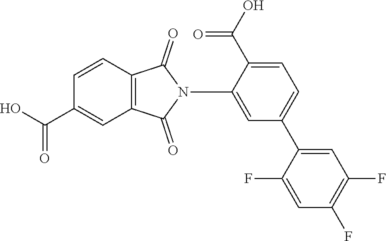 [SMILES: O=C(O)c1ccc2c(c1)C(=O)N(c1cc(-c3cc(F)c(F)cc3F)ccc1C(=O)O)C2=O]

271. 2-(4-Carboxy-2′,4′,5′-trifluoro[1,1′-biphenyl]-3-yl)-1,3-dioxo-2,3-dihydro-1H-isoindole-5-carboxylic acid was prepared as described in synthetic procedure K from trimellitic anhydride and 3-amino-2′,4′,5′-trifluoro[1,1′-biphenyl]-4-carboxylic acid (was obtained as described in synthetic procedure A followed by synthetic procedure B from methyl 2-amino-4-bromobenzoate and 2,4,5-trifluorophenylboronic acid); MS m/z: (M+H)⁺ calculated for C₂₂H₁₀F₃NO₆: 442.31. found 442.28. LC/MS retention time: 2.20 minutes.

### Example 141: 2-(4-Carboxy-4′-methyl[1,1′-biphenyl]-3-yl)-1,3-dioxo-2,3-dihydro-1H-isoindole-5-carboxylic acid

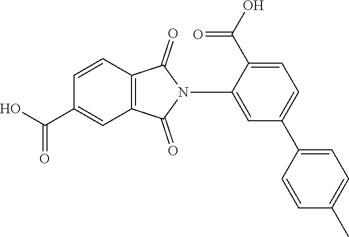 [SMILES: Cc1ccc(-c2ccc(C(=O)O)c(N3C(=O)c4ccc(C(=O)O)cc4C3=O)c2)cc1]

272. 2-(4-Carboxy-4′-methyl[1,1′-biphenyl]-3-yl)-1,3-dioxo-2,3-dihydro-1H-isoindole-5-carboxylic acid was prepared as described in synthetic procedure K from trimellitic anhydride and 3-amino-4′-methyl[1,1′-biphenyl]-4-carboxylic acid (was obtained as described in synthetic procedure A followed by synthetic procedure B from methyl 2-amino-4-bromobenzoate and 4-methylphenylboronic acid); MS m/z: (M+H)⁺ calculated for C₂₂H₁₅NO₆: 402.38. found 401.76. LC/MS retention time: 2.20 minutes.

### Example 142: 2-(4-Carboxy-2′,4′-dichloro[1,1′-biphenyl]-3-yl)-1,3-dioxo-2,3-dihydro-1H-isoindole-5-carboxylic acid

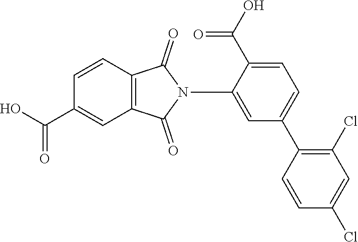 [SMILES: O=C(O)c1ccc2c(c1)C(=O)N(c1cc(-c3ccc(Cl)cc3Cl)ccc1C(=O)O)C2=O]

273. 2-(4-Carboxy-2′,4′-dichloro[1,1′-biphenyl]-3-yl)-1,3-dioxo-2,3-dihydro-1H-isoindole-5-carboxylic acid was prepared as described in synthetic procedure K from trimellitic anhydride and 3-amino-2′,4′-dichloro[1,1′-biphenyl]-4-carboxylic acid (was obtained as described in synthetic procedure A followed by synthetic procedure B from methyl 2-amino-4-bromobenzoate and 2,4-dichlorophenylboronic acid); MS m/z: (M+H)⁺ calculated for C₂₂H₁₁Cl₂NO₆: 457.24. found 456.08. LC/MS retention time: 2.39 minutes.

### Example 143: 2-(4-Carboxy-4′-chloro-3′-fluoro[1,1′-biphenyl]-3-yl)-1,3-dioxo-2,3-dihydro-1H-isoindole-5-carboxylic acid

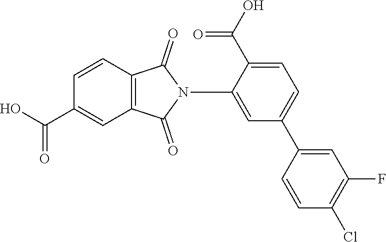 [SMILES: O=C(O)c1ccc2c(c1)C(=O)N(c1cc(-c3ccc(Cl)c(F)c3)ccc1C(=O)O)C2=O]

274. 2-(4-Carboxy-4′-chloro-3′-fluoro[1,1′-biphenyl]-3-yl)-1,3-dioxo-2,3-dihydro-1H-isoindole-5-carboxylic acid was prepared as described in synthetic procedure K from trimellitic anhydride and 3-amino-3′-fluoro-4′-chloro[1,1′-biphenyl]-4-carboxylic acid (was obtained as described in synthetic procedure A followed by synthetic procedure B from methyl 2-amino-4-bromobenzoate and 4-chloro-3-fluorophenylboronic acid); MS m/z: (M+H)⁺ calculated for C₂₂H₁₁FClNO₆: 440.79. found 439.88. LC/MS retention time: 2.28 minutes.

### Example 144: 2-(4-Carboxy-3′-fluoro-4′-methoxy[1,1′-biphenyl]-3-yl)-1,3-dioxo-2,3-dihydro-1H-isoindole-5-carboxylic acid

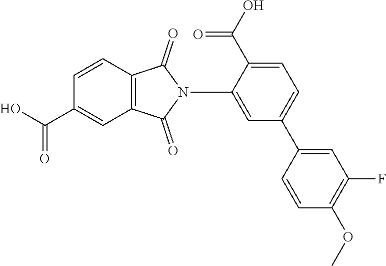 [SMILES: COc1ccc(-c2ccc(C(=O)O)c(N3C(=O)c4ccc(C(=O)O)cc4C3=O)c2)cc1F]

275. 2-(4-Carboxy-3′-fluoro-4′-methoxy[1,1′-biphenyl]-3-yl)-1,3-dioxo-2,3-dihydro-1H-isoindole-5-carboxylic acid was prepared as described in synthetic procedure K from trimellitic anhydride and 3-amino-3′-fluoro-4′-methoxy[1,1′-biphenyl]-4-carboxylic acid (was obtained as described in synthetic procedure A followed by synthetic procedure B from methyl 2-amino-4-bromobenzoate and 3-fluoro-4-methoxyphenylboronic acid); MS m/z: (M+H)⁺ calculated for C₂₃H₁₄FNO₇: 436.37. found 435.97. LC/MS retention time: 2.02 minutes.

### Example 144a: 2-(4-Carboxy-3′,4′-difluoro[1,1′-biphenyl]-3-yl)-6-hydroxy-1,3-dioxo-2,3-dihydro-1H-isoindole-5-carboxylic acid

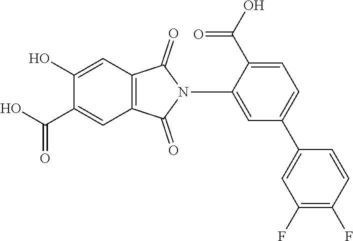 [SMILES: O=C(O)c1cc2c(cc1O)C(=O)N(c1cc(-c3ccc(F)c(F)c3)ccc1C(=O)O)C2=O]

276. 2-(4-Carboxy-3′,4′-difluoro[1,1′-biphenyl]-3-yl)-6-hydroxy-1,3-dioxo-2,3-dihydro-1H-isoindole-5-carboxylic acid was prepared by modified Synthetic Procedure K. The mixture of 5-hydroxybenzene-1,2,4-tricarboxylic acid (Intermediate 5b, 36 mg; 0.16 mmol) and 3-amino-3′,4′-difluoro[1,1′-biphenyl]-4-carboxylic acid (Intermediate 1d, 40 mg, 0.16 mmol) was stirred in 3 ml isobutyric acid at microwave irradiation at 175° C. for 3 h. After reaction is completed the mixture was poured into water (25-40 ml). The precipitate was collected by filtration, dried under high vacuum and sent for HPLC purification. MS m/z: (M+H)⁺ calculated for C₂₂H₁₁F₂NO₇: 440.05. found 440.77. LC/MS retention time: 2.09 minutes.

### Example 144b: 3-[5-Chloro-1,3-dioxo-6-(1H-1,2,3-triazol-5-yl)-2,3-dihydro-1H-isoindol-2-yl]-3′,4′-difluoro[1,1′-biphenyl]-4-carboxylic Acid

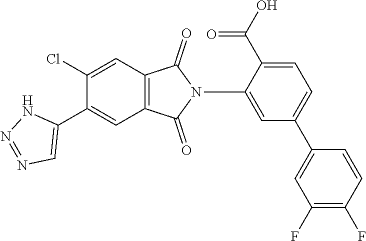 [SMILES: O=C(O)c1ccc(-c2ccc(F)c(F)c2)cc1N1C(=O)c2cc(Cl)c(-c3cnn[nH]3)cc2C1=O]

277. 3-[5-Chloro-1,3-dioxo-6-(1H-1,2,3-triazol-5-yl)-2,3-dihydro-1H-isoindol-2-yl]-3′,4′-difluoro[1,1′-biphenyl]-4-carboxylic acid was prepared by modified Synthetic Procedure L. The reaction mixture of 4-chloro-5-(1H-1,2,3-triazol-4-yl)phthalic acid (Intermediate 5a, 0.094 mmol) and 3-amino-3′,4′-difluoro[1,1′-biphenyl]-4-carboxylic acid (Intermediate 1d, 0.094 mmol) in acetic acid (2 ml) was stirred at microwave irradiation at 175° C. for 3.5 h. Acetic acid was evaporated and the residue was purified by prep-HPLC to give 3-[5-chloro-1,3-dioxo-6-(1H-1,2,3-triazol-4-yl)-2,3-dihydro-1H-isoindol-2-yl]-3′,4′-difluoro[1,1′-biphenyl]-4-carboxylic acid. MS (m/z): (M+H)⁺ calculated for C₂₃H₁₁ClF₂N₄O₄: 481.81. found 481.20. LC/MS retention time: 2.18 minutes.

### Example 144c: 3-[5-Chloro-1,3-dioxo-6-(1H-1,2,3-triazol-5-yl)-2,3-dihydro-1H-isoindol-2-yl][1,1′-biphenyl]-4-carboxylic Acid

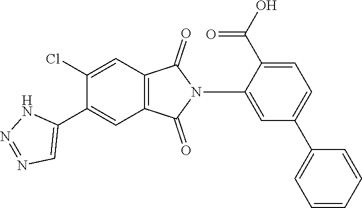 [SMILES: O=C(O)c1ccc(-c2ccccc2)cc1N1C(=O)c2cc(Cl)c(-c3cnn[nH]3)cc2C1=O]

278. 3-[5-Chloro-1,3-dioxo-6-(1H-1,2,3-triazol-5-yl)-2,3-dihydro-1H-isoindol-2-yl][1,1′-biphenyl]-4-carboxylic acid was prepared by modified Synthetic Procedure L, similarly to example 144b, from 4-chloro-5-(1H-1,2,3-triazol-4-yl)phthalic acid (Intermediate 5a) and 3-amino[1,1′-biphenyl]-4-carboxylic acid. MS (m/z): (M+H)⁺ calculated for C₂₃H₁₃ClN₄O₄: 445.61. found 445.99. LC/MS retention time: 2.23 minutes.

Examples of preparation of some compounds of Formula (VII)

For the purposes of examples of preparation of some compounds of Formula (VII) the numbering of general procedures and examples restarted from 1. The references in description of preparation of some compounds of Formula (VII) to numbered procedures and examples mean the procedures and examples numbered after the restart of numbering. For example, Step 18.3 Preparation of methyl 2-([1,1′-biphenyl]-3-carboxamido)-4-chloro-5-((trimethylsilyl)ethynyl)benzoate and methyl 2-([1,1′-biphenyl]-3-carboxamido)-4-chloro-5-ethynylbenzoate

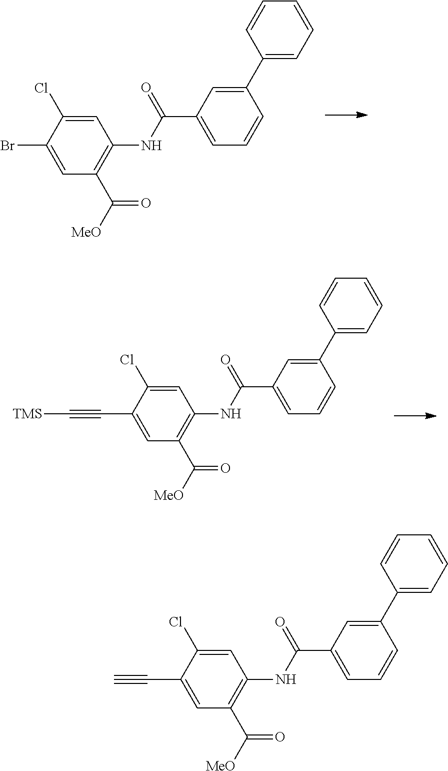 [SMILES: COC(=O)c1cc(Br)c(Cl)cc1NC(=O)c1cccc(-c2ccccc2)c1 ; COC(=O)c1cc(C#C[Si](C)(C)C)c(Cl)cc1NC(=O)c1cccc(-c2ccccc2)c1 ; C#Cc1cc(C(=O)OC)c(NC(=O)c2cccc(-c3ccccc3)c2)cc1Cl]

suggests “Using General Procedure 3, methyl 2-([1,1′-biphenyl]-3-carboxamido)-5-bromo-4-chlorobenzoate (0.265 g) . . . ”, the General Procedure 3 of this “Examples of preparation of some compounds of Formula (VII)” part is meant, which is described below: “General procedure #3: Ethynylation of Halo Benzoic Acids (Esters)

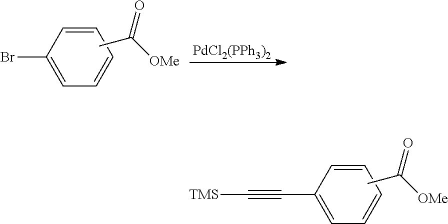

The bromobenzoic ester . . . ”

General Procedure #1: Halo Anthranilic Acid (Ester)—Aryl Boronic Acid (Ester) Coupling

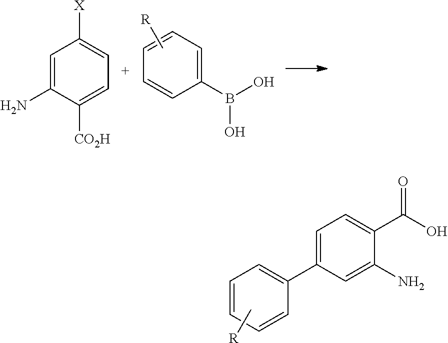

The haloanthranilic acid (ester) (1 equiv) is dissolved in DMF (50 mL/1 mmol halide). To this solution is added the aryl boronic acid (ester) (1.3 equiv), the palladium catalyst (Pd(Ph₃)₄ or PdCl₂(dppf), 0.1-0.35 equiv), and a carbonate base (Cs₂CO₃ or K₂CO₃, 2 equiv). The mixture is heated overnight at 100-110° C. and then cooled. The DFM is evaporated under reduced pressure, NaOH is added (4 M, 80 mL/mmol aryl halide) and the mixture is extracted with ethyl acetate. After separation of the layers, the ethyl acetate is further extracted with 4 M NaOH (2 x). The combined aqueous layers are acidified with HCl (conc.) to pH ˜2 and then filtered. The precipitated acid product is taken up in ethyl acetate, filtered from any solids, washed with water, dried over sodium sulfate, concentrated and then dried under vacuum to afford the pure acid.

General Procedure #2: Ring Opening of 2,4-Dioxo-1,4-Dihydro-2H-Benzo[d][1,3]Oxazine-7-Carboxylic Acid by Substituted Anthranilic Acids

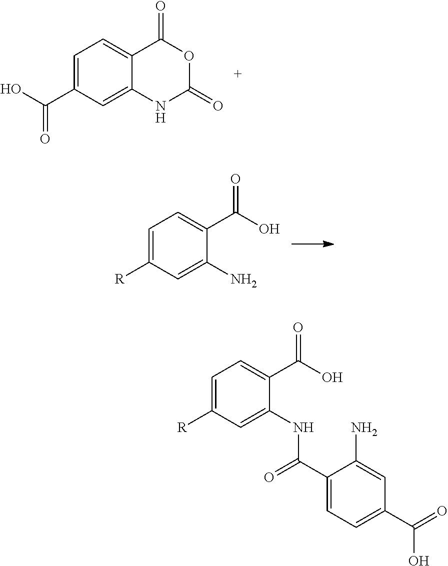

2,4-dioxo-1,4-dihydro-2H-benzo[d][1,3]oxazine-7-carboxylic acid (2 equiv) and the substituted anthranilic acid (1 equiv) are mixed in a mixture of water and dioxane (1:5) and heated overnight at 110° C. If the reaction is incomplete, more 2,4-dioxo-1,4-dihydro-2H-benzo[d][1,3]oxazine-7-carboxylic acid is added and the reaction is heated at 110° C. until it is judged complete. Upon cooling, the solution is poured into water and acidified with 0.2 N HCl to pH ˜2 and extracted with ethyl acetate (3×). The combined organic layers are washed with water (2×), dried over sodium sulfate, concentrated and sent for purification by preparative HPLC to afford the pure 3-amino-4-((2-carboxyphenyl)carbamoyl) substituted benzoic acid.

General Procedure #3: Ethynylation of Halo Benzoic Acids (Esters)

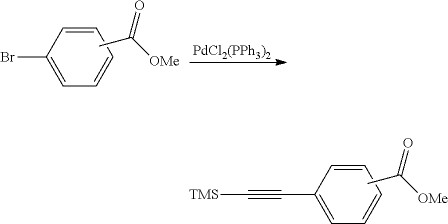

The bromobenzoic ester (1 equiv) is dissolved in DMF (10 mL/mmol) and then treated with PdCl₂(PPh₃)₂ (15 mole %), CuI (20 mol %), TMS acetylene (10 equiv) and triethylamine (10 equiv). The mixture is heated in a microwave reactor at 100° C. for 2.5 h and cooled. The mixture is poured into water and extracted with ethyl acetate (3×). The combined organic extracts are washed with water, dried over sodium sulfate, filtered and concentrated to dryness. The residue is purified by flash chromatography to afford the TMS-ethynyl benzoic ester.

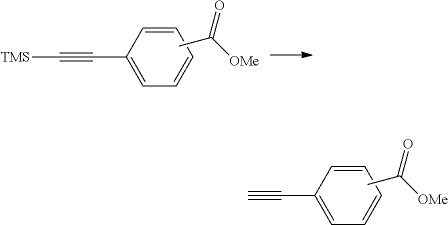

The purified TMS-ethynyl benzoic ester (1 equiv) is dissolved in a mixture of methanol-dichloromethane (1:1) and treated with potassium carbonate (2 equiv) at room temperature for 1 h. The reaction mixture is diluted with ethyl acetate and filtered through a pad of Celite. The solution is poured into saturated ammonium chloride solution and extracted 3× with ethyl acetate, dried over sodium sulfate, filtered and concentrated to dryness.

General Procedure #4: Preparation of Methyl 2-Substituted-5-(1H-1,2,3-triazol-5-yl)benzoates from Methyl 5-ethynyl-2-Substituted-benzoates

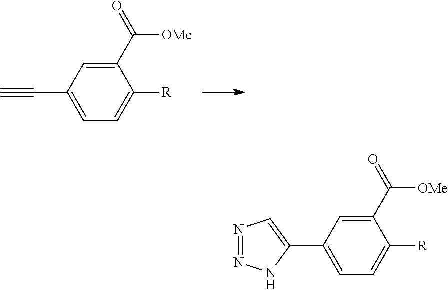

The ethynylbenzoate ester (1 equiv) is treated with TMS azide (3 equiv) and CuI (20 mole %) in a mixture of DMF and methanol (10:1) at 100° C. in a microwave reactor for 4 h. The cooled reaction is poured into water and the solid thus formed is collected by filtration.

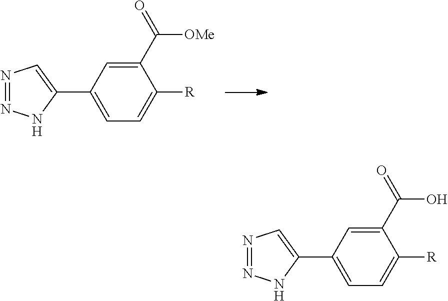

The ester thus formed (1 equiv) is dissolved in methanol-THF (1:2) and heated at 40° C. with sodium hydroxide (10 equiv, 2N) for 8 h. HCl is used to acidify the cooled reaction mixture to pH-2, water is added and the mixture is evaporated to dryness and sent for preparative HPLC purification to afford pure acid.

General Procedure #5: Reaction of Arylamines with Phthalic Acids to Form N-Aryl-Phthalimides

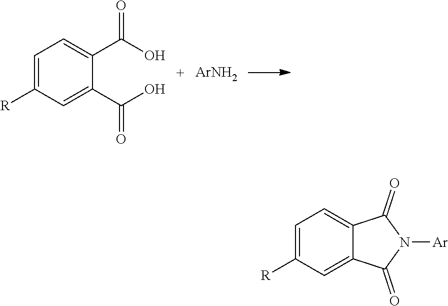

The substituted phthalic acid (1 equiv) and the arylamine (1 equiv) are dissolved in acetic acid (10-12 mL/mmole amine)and heated in a microwave reactor at 120° C. for 6-24 h. The solvent is evaporated to dryness and the phthalimide thus formed is purified by flash chromatography using dichloromethane-methanol mixtures to afford pure product.

General Procedure #6: Ring Opening of N-Aryl Phthalimides with Ammonia (Amines)

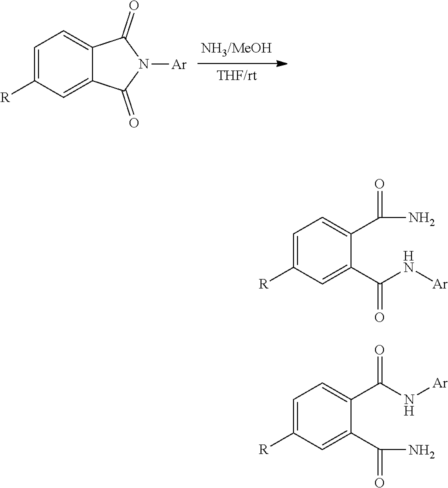

The N-aryphthalimide (1 equiv) is dissolved in THF (20-25 mL/mmole) and treated with 7M ammonia gas in methanol (10 equiv), stirring at room temperature for 15-30 min. The excess ammonia gas is largely eliminated by purging with nitrogen gas for a few minutes and the solution is poured into water and extracted 3× with ethyl acetate. The combined organic layers are washed with water (2×), dried over sodium sulfate, filtered, concentrated and then purified by preparative HPLC to separate the two resulting region isomers.

General Procedure #7: Ring Opening of N-Aryl Phthalimides with Sodium Methoxide

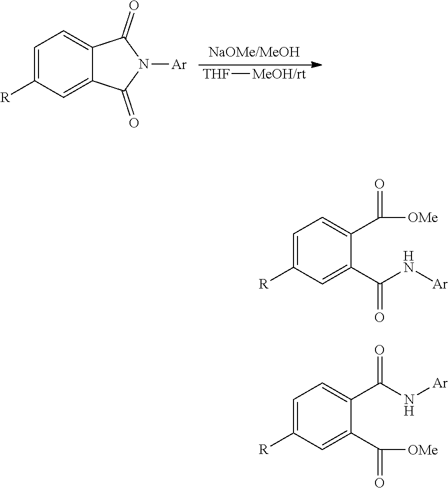

The N-aryphthalimide (1 equiv) is dissolved in THF-MeOH ((3:2), 20-25 mL/mmole) and treated with NaOMe in methanol (25% w/w, 3 equiv), stirring at room temperature for 15-30 min. The solution is poured into ethyl acetate-water and extracted 3× with ethyl acetate. The combined organic layers are washed with water (2×), dried over sodium sulfate, filtered, concentrated and then purified by preparative HPLC to separate the two resulting region isomers.

General Procedure #8: Amidation of N-Arylphthalimido-5-Carboxylic Acids

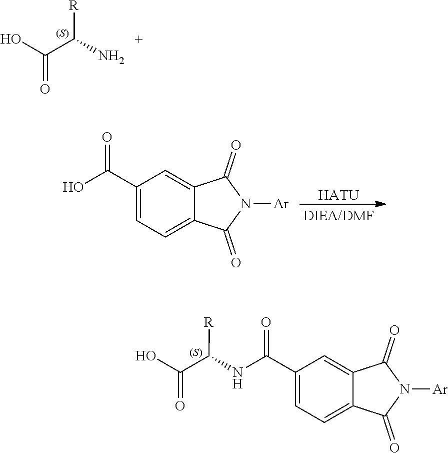

The N-arylphthalimido-5-carboxylic acid (1 equiv) is dissolved in anhydrous DMF (10-15 mL/mmol)and treated with HATU (1.3 equiv), the aminoester (1.05 equiv) and diisopropylethylamine(2.5 equiv) at room temperature for 2-6 h. When the reaction is complete, the mixture is poured into water and extracted (3×) with ethyl acetate-THF (1:1 mixture, 3×). The combined organic layers are washed with water followed by brine, dried over sodium sulfate, filtered, concentrated and purified by flash chromatography using hexane-ethyl acetate mixtures to afford the pure amide.

General Procedure #9: Ring Opening of Substituted Phthalic Anhydrides with Substituted Anthranilic Acids

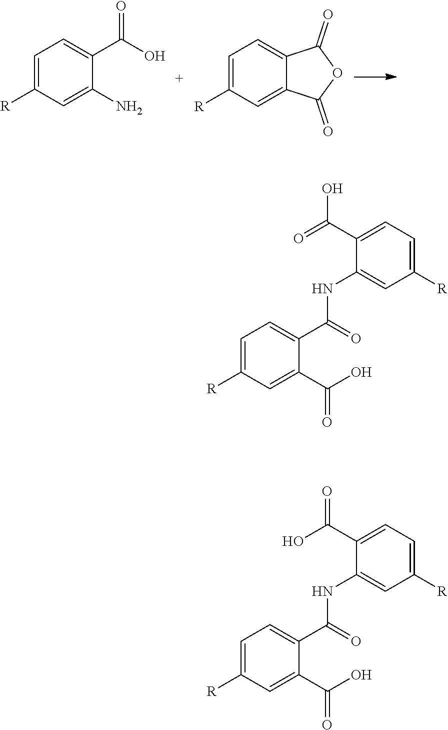

The Substituted phthalic anhydride (1.03 equiv) is dissolved in acetic acid (7 mL/mmol) and treated with the substituted anthranilic acid at 80° C., stirring for 1 h. The cooled solution is evaporated to dryness and the two isomers are separated and purified by preparative HPLC.

General Procedure 10: Benzoylation of Anthranilic Ester

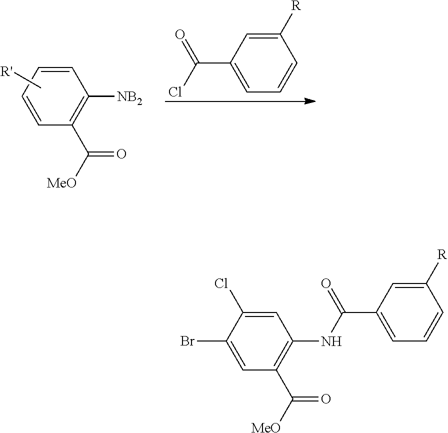

The acid chloride (1 equiv) is dissolved in THF (10 mL/g) and treated with the anthranilic ester (1 equiv) and triethylamine (1.5 equiv) at room temperature overnight. The solvent is removed by evaporation, the product is triturated with THF, washed with water and then dried under high vacuum to afford the crude benzamide which is used without further purification.

### Example 1: 2-({4-carboxy-4′-fluoro-[1,1′-biphenyl]-3-yl}carbamoyl)benzene-1,4-dicarboxylic acid (GO-0000218)

2-({4-carboxy-4′-fluoro-[1,1′-biphenyl]-3-yl}carbamoyl)benzene-1,4-dicarboxylic acid was prepared in several steps.

### Step 1.1 Preparation of methyl 3-amino-4′-fluoro-[1,1′-biphenyl]-4-carboxylate

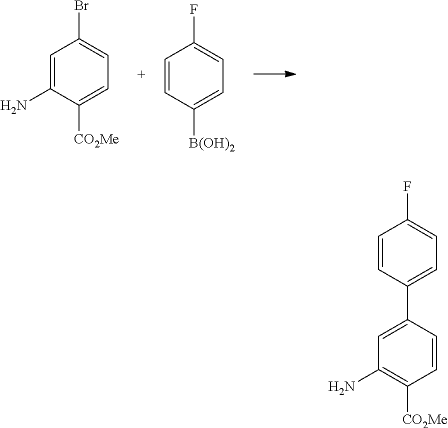 [SMILES: COC(=O)c1ccc(Br)cc1N ; OB(O)c1ccc(F)cc1 ; COC(=O)c1ccc(-c2ccc(F)cc2)cc1N]

A solution of methyl 2-amino-4-bromobenzoate (1.15 g, 5 mmol) and (4-fluorophenyl)boronic acid-(0.770 g, 5.5 mmol) in dioxane (17 mL) and water (4 mL) was treated with tetrakis(triphenylphosphine)palladium(0) (0.289 g, 0.25 mmol) and potassium carbonate (1.38 g, 10 mmol) and heated in a microwave reactor at 120 C for 3 h. The cooled reaction mixture was poured into water and extracted with ethyl acetate (3×). The combined organic solution was dried over sodium sulfate, filtered, and evaporated to dryness. The residue was purified by flash chromatography (silica gel, ethyl acetate 0-80% in hexane) to afford pure methyl 3-amino-4′-fluoro-[1,1′-biphenyl]-4-carboxylate (1.12 g, 92% yield).

### Step 1.2 Preparation of 4-((4′-fluoro-4-(methoxycarbonyl)-[1,1′-biphenyl]-3-yl)carbamoyl)isophthalic acid and 2-((4′-fluoro-4-(methoxycarbonyl)-[1,1′-biphenyl]-3-yl)carbamoyl)terephthalic acid

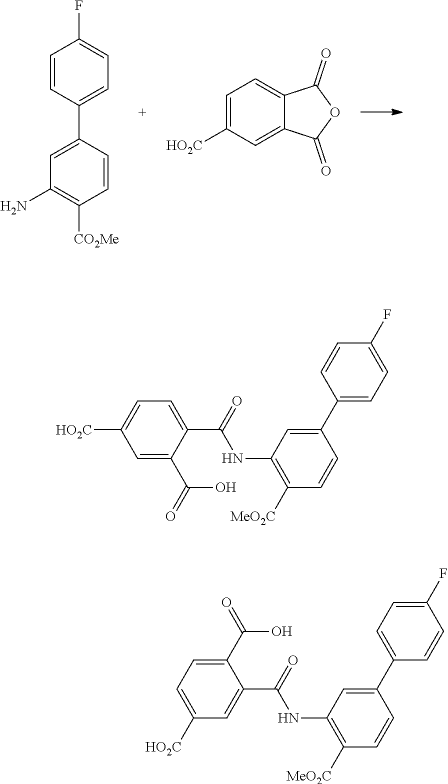 [SMILES: COC(=O)c1ccc(-c2ccc(F)cc2)cc1N ; O=C(O)c1ccc2c(c1)C(=O)OC2=O ; COC(=O)c1ccc(-c2ccc(F)cc2)cc1NC(=O)c1ccc(C(=O)O)cc1C(=O)O ; COC(=O)c1ccc(-c2ccc(F)cc2)cc1NC(=O)c1cc(C(=O)O)ccc1C(=O)O]

A mixture of methyl 3-amino-4′-fluoro-[1,1′-biphenyl]-4-carboxylate (0.250 g, 1.02 mmol) and 1,3-dioxo-1,3-dihydroisobenzofuran-5-carboxylic acid (0.235 g, 1.22 mmol) was dissolved in THF (16 mL) and treated with diisopropylethylamine (0.44 mL, 2.54 mmol) and heated in a sealed pressure vessel at 100° C. for 3.5 h. After removal of the solvent, the residue was redissolved in ethyl acetate, washed with HCl (0.2N), followed by water and brine, then dried over sodium sulfate, filtered and evaporated to dryness. The residue was purified by flash chromatography (silica gel, methanol 0-15% in dichloromethane) to afford a mixture of 4-((4′-fluoro-4-(methoxycarbonyl)-[1,1′-biphenyl]-3-yl)carbamoyl)isophthalic acid and its isomer, 2-((4′-fluoro-4-(methoxycarbonyl)-[1,1′-biphenyl]-3-yl)carbamoyl)terephthalic acid (0.401 g. 90% yield). This mixture was used in the following step without further purification.

### Step 1.3 Preparation of 4-((4-carboxy-4′-fluoro-[1,1′-biphenyl]-3-yl)carbamoyl)isophthalic acid and 2-((4-carboxy-4′-fluoro-[1,1′-biphenyl]-3-yl)carbamoyl)terephthalic acid

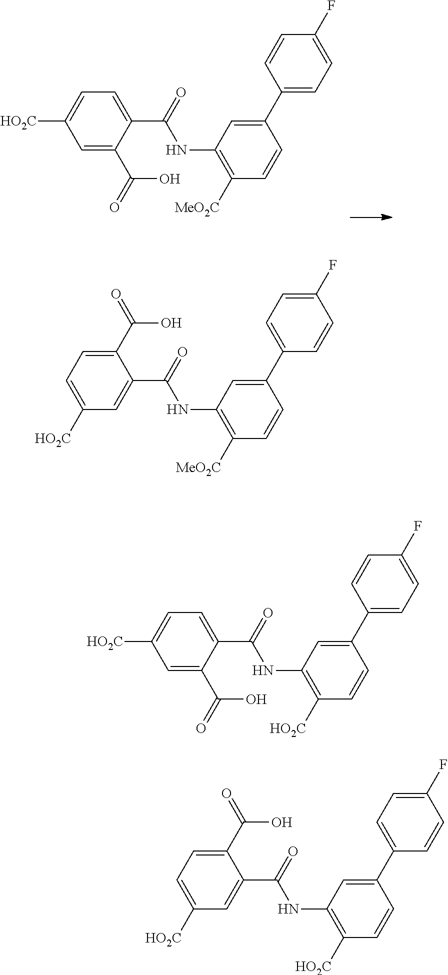 [SMILES: COC(=O)c1ccc(-c2ccc(F)cc2)cc1NC(=O)c1ccc(C(=O)O)cc1C(=O)O ; COC(=O)c1ccc(-c2ccc(F)cc2)cc1NC(=O)c1cc(C(=O)O)ccc1C(=O)O ; O=C(O)c1ccc(C(=O)Nc2cc(-c3ccc(F)cc3)ccc2C(=O)O)c(C(=O)O)c1 ; O=C(O)c1ccc(C(=O)O)c(C(=O)Nc2cc(-c3ccc(F)cc3)ccc2C(=O)O)c1]

The mixture of isomers obtained in the previous step (4-((4′-fluoro-4-(methoxycarbonyl)-[1,1′-biphenyl]-3-yl)carbamoyl)isophthalic acid 2-((4′-fluoro-4-(methoxycarbonyl)-[1,1′-biphenyl]-3-yl)carbamoyl)terephthalic acid was dissolved in a mixture of methanol (6 mL) and THF (6 mL) and treated with sodium hydroxide (1.83 mL, 2N, 3.67 mmol) at room temperature for 80 min. The pH of the solution was adjusted to ˜2 by the addition of HCl (0.2N) and then extracted with ethyl acetate (3×). The combined organic solution was washed with water and brine, concentrated and dried under vacuum. Preparative HPLC afforded pure 4-((4-carboxy-4′-fluoro-[1,1′-biphenyl]-3-yl)carbamoyl)isophthalic acid and 2-((4-carboxy-4′-fluoro-[1,1′-biphenyl]-3-yl)carbamoyl)terephthalic acid. The target compound was selected as the isomer, which matched the NMR spectra.

1H NMR (500 MHz, DMSO-d6) δ ppm 7.37 (t, J=8.79 Hz, 2H) 7.52 (d, J=8.24 Hz, 1H) 7.70-7.81 (m, 3H) 7.96 (d, J=8.24 Hz, 1H) 8.10 (d, J=8.24 Hz, 1H) 8.17 (d, J=8.24 Hz, 1H) 8.23 (s, 1H) 8.85 (br. s., 1H)

### Example 4: 3-(2-amino-4-carboxybenzamido)-[1,1′-biphenyl]-4-carboxylic acid (GO-0000228)

3-(2-amino-4-carboxybenzamido)-[1,1′-biphenyl]-4-carboxylic acid was prepared in several steps.

### Step 4.1 Preparation of 3-amino-[1,1′-biphenyl]-4-carboxylic Acid

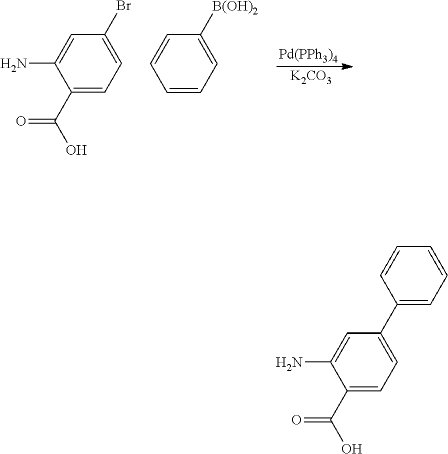

Using the general procedure General Procedure #1 for haloanthranilic acid (ester)-aryl boronic acid (ester) coupling, 2-amino-4-bromobenzoic acid (2 g) was coupled with phenylboronic acid (1.46 g) using Pd(PPh₃)₄ and potassium carbonate (18.6 mL, 1 M) to afford 3-amino-[1,1′-biphenyl]-4-carboxylic acid (1.1 g, 56% yield).

### Step 4.2 Preparation of 3-(2-amino-4-carboxybenzamido)-[1,1′-biphenyl]-4-carboxylic acid

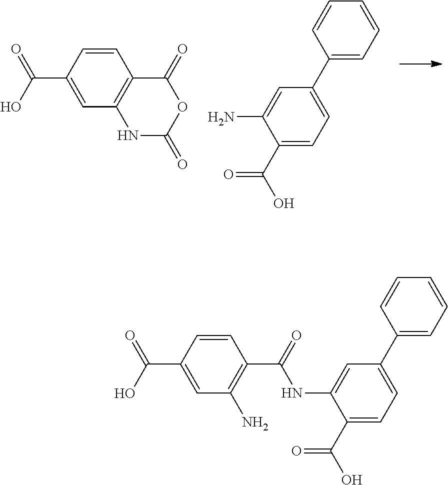 [SMILES: O=C(O)c1ccc2c(=O)oc(=O)[nH]c2c1 ; Nc1cc(-c2ccccc2)ccc1C(=O)O ; Nc1cc(C(=O)O)ccc1C(=O)Nc1cc(-c2ccccc2)ccc1C(=O)O]

Using this General Procedure #2 for ring opening of 2,4-dioxo-1,4-dihydro-2 h-benzo[d][1,3]oxazine-7-carboxylic acid by substituted anthranilic acids, 3-amino-[1,1′-biphenyl]-4-carboxylic acid (0.1 g) was reacted with 2,4-dioxo-1,4-dihydro-2H-benzo[d][1,3]oxazine-7-carboxylic acid (0.8 g) in water-dioxane (2.4 mL, 1:5) to afford 3-(2-amino-4-carboxybenzamido)-[1,1′-biphenyl]-4-carboxylic acid (0.062 g, 30%).

1H NMR (500 MHz, DMSO-d6) δ ppm 7.15 (d, J=8.24 Hz, 1H) 7.46 (t, J=7.69 Hz, 3H) 7.49 (br. s., 1H) 7.53 (t, J=7.41 Hz, 3H) 7.72 (d, J=8.24 Hz, 2H) 8.12 (d, J=8.24 Hz, 1H) 8.99 (s, 1H)

### Example 5: 4-({4-carboxy-[1,1′-biphenyl]-3-yl}carbamoyl)benzene-1,3-dicarboxylic acid (GO-0000229)

4-({4-carboxy-[1,1′-biphenyl]-3-yl}carbamoyl)benzene-1,3-dicarboxylic acid was prepared in one step.

### Step 5.1 Preparation of 4-({4-carboxy-[1,1′-biphenyl]-3-yl}carbamoyl)benzene-1,3-dicarboxylic Acid

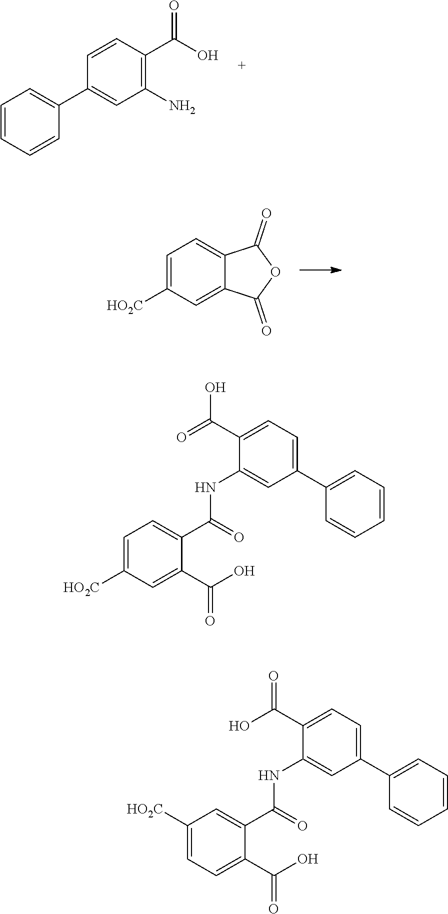 [SMILES: Nc1cc(-c2ccccc2)ccc1C(=O)O ; O=C(O)c1ccc2c(c1)C(=O)OC2=O ; O=C(O)c1ccc(C(=O)Nc2cc(-c3ccccc3)ccc2C(=O)O)c(C(=O)O)c1 ; O=C(O)c1ccc(C(=O)O)c(C(=O)Nc2cc(-c3ccccc3)ccc2C(=O)O)c1]

Using General Procedure #9, 3-amino-[1,1′-biphenyl]-4-carboxylic acid (0.300 g) was reacted with 1,3-dioxo-1,3-dihydroisobenzofuran-5-carboxylic acid (0.279 g) to afford 4-((4-carboxy-[1,1′-biphenyl]-3-yl)carbamoyl)isophthalic acid (GO-0000229) and 2-((4-carboxy-[1,1′-biphenyl]-3-yl)carbamoyl)terephthalic acid after separation and purification. The target compound was selected as the isomer, which matched the NMR spectra.

1H NMR (500 MHz, DMSO-d6) δ ppm 7.44-7.49 (m, 1H) 7.52-7.62 (m, 4H) 7.72 (d, J=7.69 Hz, 2H) 7.96 (d, J=8.24 Hz, 1H) 8.11 (d, J=8.24 Hz, 1H) 8.17 (d, J=7.69 Hz, 1H) 8.23 (s, 1H) 8.88 (br. s., 1H)

### Example 6: 3-((2-carboxy-4-(1H-1,2,3-triazol-4-yl)benzyl)amino)-3′,4′-difluoro-[1,1′-biphenyl]-4-carboxylic acid (GO-0000286)

3-((2-carboxy-5-(1H-1,2,3-triazol-5-yl)benzyl)amino)-3′,4′-difluoro-[1,1′-biphenyl]-4-carboxylic acid was prepared in several steps.

### Step 6.1 Preparation of methyl 5-bromo-2-(bromomethyl)benzoate

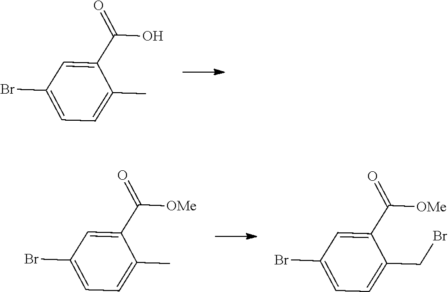

5-bromo-2-methylbenzoic acid (1.53 g) was esterified by dissolving in methanol (35 mL) catalyzed by the addition of HCl (30 drops) and heating at reflux for 7 h. Evaporation of the solvent and drying under vacuum afforded pure methyl 5-bromo-2-methylbenzoate. The crude material (1.5 g, 6.55 mmol) was dissolved in carbon tetrachloride (35 mL) and treated with NBS (1.4 g, 7.8 mmol) and AIBN (0.0065 g, 6% molar equivalent) at reflux for 5.5 h. The cooled mixture was poured into water and extracted with dichloromethane (3×), dried over sodium sulfate, concentrated and purified by flash chromatography (silica gel, hexane: dichloromethane (0 to 30%) to afford methyl 5-bromo-2-(bromomethyl)benzoate (1.51 g, 75% yield).

### Step 6.2 Preparation of methyl 3-amino-3′,4′-difluoro-[1,1′-biphenyl]-4-carboxylate

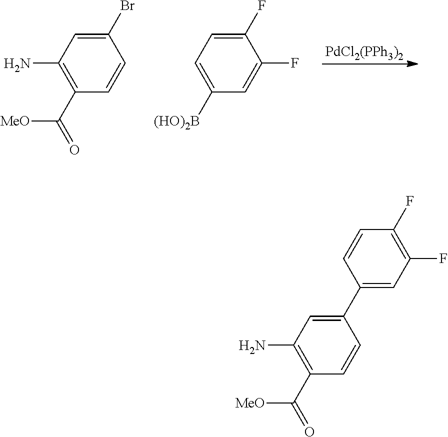 [SMILES: COC(=O)c1ccc(Br)cc1N ; OB(O)c1ccc(F)c(F)c1 ; COC(=O)c1ccc(-c2ccc(F)c(F)c2)cc1N ; "PdCl2(PPh3)2"]

Using the General Procedure #1 for haloanthranilic acid (ester)-aryl boronic acid (ester) coupling, methyl 2-amino-4-bromobenzoate (1.15 g) is reacted with (3,4-difluorophenyl)boronic acid (0.869 g), PdCl₂(PPh₃)₂ (0.289 g) and potassium carbonate (1.38 g) in water-dioxane (4 mL+12 mL) in a microwave reactor at 120° C. for 3 h. After purification by flash chromatography (silica gel, hexane-ethyl acetate (0 to 80%) to afford pure methyl 3-amino-3′,4′-difluoro-[1,1′-biphenyl]-4-carboxylate (1 g, 77% yield).

### Step 6.3 Preparation of methyl 3-((4-bromo-2-(methoxycarbonyl)benzyl)amino)-3′,4′-difluoro-[1,1′-biphenyl]-4-carboxylate

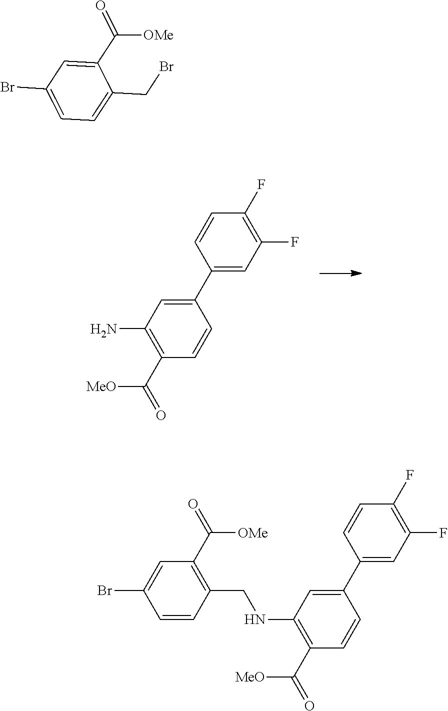 [SMILES: COC(=O)c1cc(Br)ccc1CBr ; COC(=O)c1ccc(-c2ccc(F)c(F)c2)cc1N ; COC(=O)c1cc(Br)ccc1CNc1cc(-c2ccc(F)c(F)c2)ccc1C(=O)OC]

Methyl 5-bromo-2-(bromomethyl)benzoate (0.865 g, 2.8 mmol) and methyl 3-amino-3′,4′-difluoro-[1,1′-biphenyl]-4-carboxylate (0.739 g, 2.8 mmol) are reacted in acetonitrile (30 mL) in the presence of potassium carbonate (0.776 g, 5.61 mmol). The mixture was heated at 80° C. for 16 h and cooled. Dilution with ethyl acetate produced a precipitate which was filtered off and washed with ethyl acetate. The organic solution was washed with water, dried over sodium sulfate, concentrated to dryness and pumped dry under high vacuum. The crude material was used directly in the next step.

### Step 6.4 Preparation of methyl 3-((4-ethynyl-2-(methoxycarbonyl)benzyl)amino)-3′,4′-difluoro-[1,1′-biphenyl]-4-carboxylate

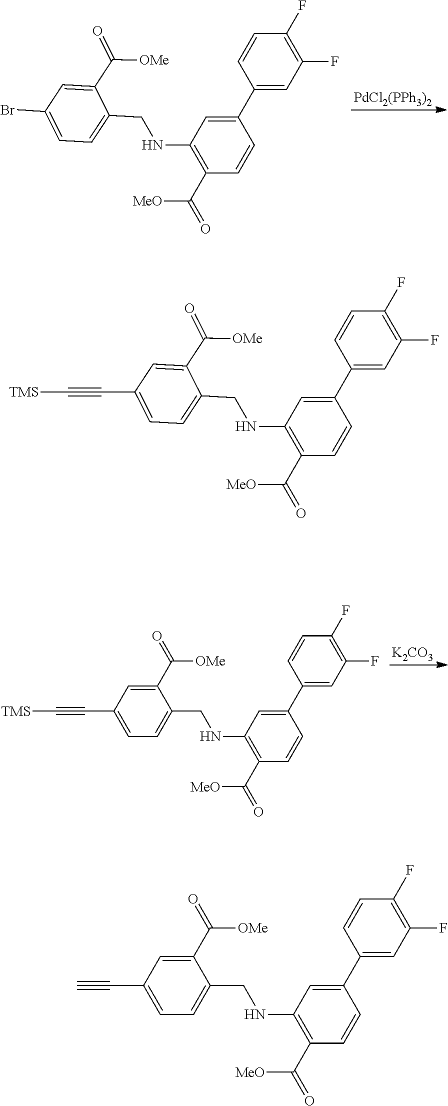

Using General Procedure #3 for the ethynylation of halo benzoic acids (esters),methyl 3-((4-bromo-2-(methoxycarbonyl)benzyl)amino)-3′,4′-difluoro-[1,1′-biphenyl]-4-carboxylate (0.700 g) was reacted with PdCl₂(PPh₃)₂ (0.150 g), Cui (0.056 G), TMS-acetylene (2 mL) and triethylamine (2 mL) to afford methyl 3′,4′-difluoro-3-((2-(methoxycarbonyl)-4-((trimethylsilyl)ethynyl)benzyl)amino)-[1,1′-biphenyl]-4-carboxylate (0.990 g) which in turn was hydrolyzed according to the procedure to afford methyl 3-((4-ethynyl-2-(methoxycarbonyl)benzyl)amino)-3′,4′-difluoro-[1,1′-biphenyl]-4-carboxylate.

### Step 6.5 Preparation of methyl 3′,4′-difluoro-3-((2-(methoxycarbonyl)-4-(1H-1,2,3-triazol-4-yl)benzyl)amino)-[1,1′-biphenyl]-4-carboxylate

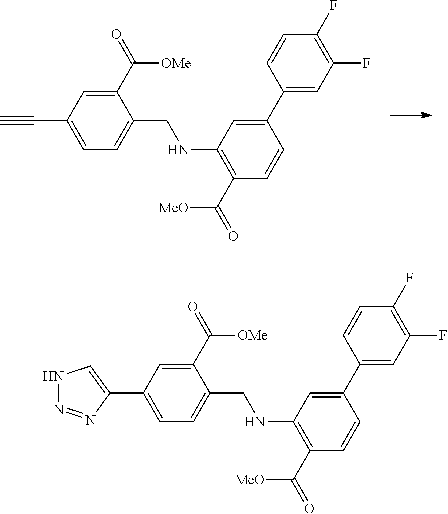 [SMILES: C#Cc1ccc(CNc2cc(-c3ccc(F)c(F)c3)ccc2C(=O)OC)c(C(=O)OC)c1 ; COC(=O)c1cc(-c2c[nH]nn2)ccc1CNc1cc(-c2ccc(F)c(F)c2)ccc1C(=O)OC]

Using General Procedure #4 for the preparation of methyl 2-substituted-5-(1 h-1,2,3-triazol-5-yl)benzoates from methyl 5-ethynyl-2-substituted-benzoates, methyl 3-((4-ethynyl-2-(methoxy-carbonyl)benzyl)amino)-3′,4′-difluoro-[1,1′-biphenyl]-4-carboxylate (0.375 g) was converted into methyl 3′,4′-difluoro-3-((2-(methoxycarbonyl)-4-(1H-1,2,3-triazol-4-yl)benzyl)amino)-[1,1′-biphenyl]-4-carboxylate (0.422 g).

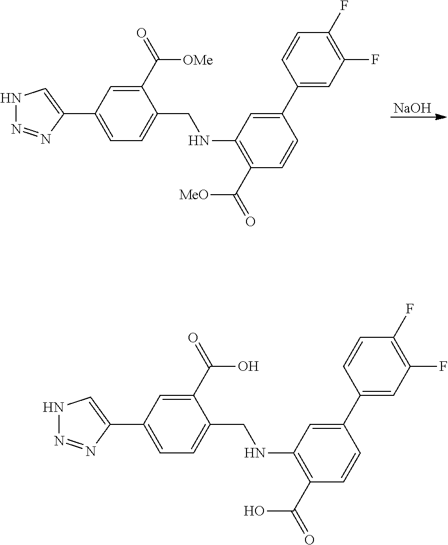 [SMILES: COC(=O)c1cc(-c2c[nH]nn2)ccc1CNc1cc(-c2ccc(F)c(F)c2)ccc1C(=O)OC ; "NaOH" ; O=C(O)c1cc(-c2c[nH]nn2)ccc1CNc1cc(-c2ccc(F)c(F)c2)ccc1C(=O)O]

The methyl 3′,4′-difluoro-3-((2-(methoxycarbonyl)-4-(1H-1,2,3-triazol-4-yl)benzyl)amino)-[1,1′-biphenyl]-4-carboxylate thus obtained (0.100 g) was hydrolyzed according to General Procedure #4 to afford pure 3-((2-carboxy-4-(1H-1,2,3-triazol-4-yl)benzyl)amino)-3′,4′-difluoro-[1,1′-biphenyl]-4-carboxylic acid. The target compound was selected as the isomer, which didn't match the NMR spectra of undesirable isomer.

### Example 7: 3-((2-carboxy-5-(1H-1,2,3-triazol-5-yl)benzyl)amino)-3′,4′-difluoro-[1,1′-biphenyl]-4-carboxylic acid (GO-0000287)

3-((2-carboxy-5-(1H-1,2,3-triazol-5-yl)benzyl)amino)-3′,4′-difluoro-[1,1′-biphenyl]-4-carboxylic acid was prepared in several steps.

### Step 7.1 Preparation of methyl 4-bromo-2-(bromomethyl)benzoate (and methyl 4-bromo-2-(dibromomethyl)benzoate)

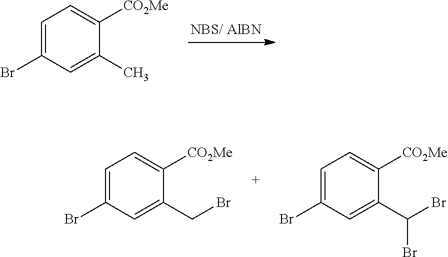

Methyl 4-bromo-2-methylbenzoate (2.17 g, 9.47 mmol) was dissolved in carbon tetrachloride (50 mL) and treated with NBS (1.68 g, 9.47 mmol) and AIBN (0.093 g, 0.568 mmol) at reflux overnight, after which time another 0.5 equiv of NBS and 0.1 g of AIBN were added and the mixture heated for an additional 5 h. The cooled reaction mixture was poured into water, and extracted with dichloromethane. The organic layer was dried, evaporated and purified by flash chromatography (silica gel, hexane-dichloromethane, 100:0 to 70:30) to afford two products. The desired methyl 4-bromo-2-(bromomethyl)benzoate (1.5 g, 53% yield) and the over brominated methyl 4-bromo-2-(dibromomethyl)-benzoate (1.3 g).

### Step 7.2 Preparation of methyl 3-((5-bromo-2-(methoxycarbonyl)benzyl)amino)-3′,4′-difluoro-[1,1′-biphenyl]-4-carboxylate

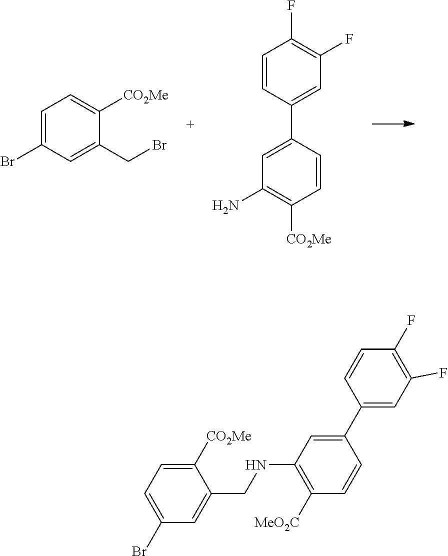 [SMILES: COC(=O)c1ccc(Br)cc1CBr ; COC(=O)c1ccc(-c2ccc(F)c(F)c2)cc1N ; COC(=O)c1ccc(Br)cc1CNc1cc(-c2ccc(F)c(F)c2)ccc1C(=O)OC]

Methyl 4-bromo-2-(bromomethyl)benzoate (0.56 g, 1.818 mmol) was dissolved in acetonitrile (20 mL) and treated with methyl 3-amino-3′,4′-difluoro-[1,1′-biphenyl]-4-carboxylate 0.479 g, 1.818 mmol) and potassium carbonate (0.503 g, 3.64 mmol). The mixture was heated at 80° C. for 23 h at which time the cooled reaction mixture was diluted with ethyl acetate and filtered to remove the solids. The filtrate was poured into water, extracted three times with ethyl acetate, dried over sodium sulfate, filtered and concentrated to afford crude methyl 3-((5-bromo-2-(methoxycarbonyl)benzyl)amino)-3′,4′-difluoro-[1,1′-biphenyl]-4-carboxylate (0.95 g) which was used in the next step without further purification.

### Step 7.3 Preparation of methyl 3′,4′-difluoro-3-((2-(methoxycarbonyl)-5-((trimethylsilyl)ethynyl)-benzyl)amino)-[1,1′-biphenyl]-4-carboxylate

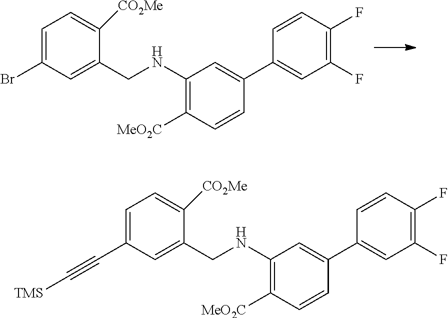 [SMILES: COC(=O)c1ccc(Br)cc1CNc1cc(-c2ccc(F)c(F)c2)ccc1C(=O)OC ; COC(=O)c1ccc(C#C[Si](C)(C)C)cc1CNc1cc(-c2ccc(F)c(F)c2)ccc1C(=O)OC]

The crude methyl 3-((5-bromo-2-(methoxycarbonyl)benzyl)amino)-3′,4′-difluoro-[1,1′-biphenyl]-4-carboxylate (0.95 g, 1.93 mmol) from the previous step was dissolved in anhydrous DMF (18 mL). To this solution was added PdCl₂(PPh₃)₂ (0.20 g, 0.28 mmol), CuI (0.073 g, 0.386 mmol, trimethylsilyl acetylene (0.95 g, 9.65 mmol) and triethylamine (1.35 mL, 9.65 mmol). The reaction mixture was heated at 100° C. in a microwave reactor for 2.5 h, at which time the cooled solution was poured into water and extracted three times with ethyl acetate. The combined organic layers were washed with water, dried over sodium sulfate, filtered, concentrated and purified by flash chromatography (silica gel, hexane:ethyl acetate, 100:0 to 80:20) to afford pure methyl 3-((5-bromo-2-(methoxycarbonyl)benzyl)amino)-3′,4′-difluoro-[1,1′-biphenyl]-4-carboxylate (0.548 g, 59% yield over 2 steps).

### Step 7.4 Preparation of methyl 3-((5-ethynyl-2-(methoxycarbonyl)benzyl)amino)-3′,4′-difluoro-[1,1′-biphenyl]-4-carboxylate

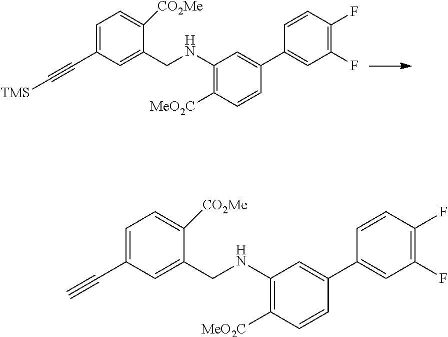 [SMILES: COC(=O)c1ccc(C#C[Si](C)(C)C)cc1CNc1cc(-c2ccc(F)c(F)c2)ccc1C(=O)OC ; C#Cc1ccc(C(=O)OC)c(CNc2cc(-c3ccc(F)c(F)c3)ccc2C(=O)OC)c1]

Methyl 3′,4′-difluoro-3-((2-(methoxycarbonyl)-5-((trimethylsilyl)ethynyl)benzyl)amino)-[1,1′-biphenyl]-4-carboxylate (0.548 g, 1.075 mmol) was dissolved in a 1:1 mixture of methanol and dichloromethane (40 mL) and treated at room temperature for 1.5 h with potassium carbonate ((0.297 g, 2.15 mmol). The reaction mixture was diluted with ethyl acetate and filtered through Celite. The resulting solution was washed with saturated ammonium chloride solution, then with water and dried over sodium sulfate. The mixture was filtered and evaporated to dryness and dried under vacuum to afford pure methyl 3-((5-ethynyl-2-(methoxycarbonyl)benzyl)amino)-3′,4′-difluoro-[1,1′-biphenyl]-4-carboxylate (0.470 g, quantitative yield)

### Step 7.5 Preparation of methyl 3′,4′-difluoro-3-((2-(methoxycarbonyl)-5-(1H-1,2,3-triazol-5-yl)benzyl)amino)-[1,1′-biphenyl]-4-carboxylate

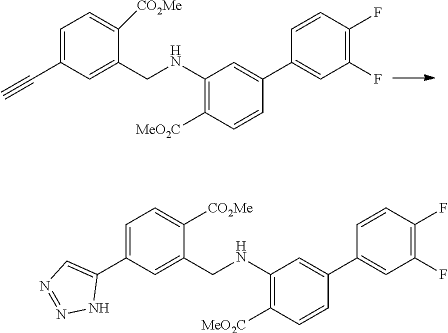 [SMILES: C#Cc1ccc(C(=O)OC)c(CNc2cc(-c3ccc(F)c(F)c3)ccc2C(=O)OC)c1 ; COC(=O)c1ccc(-c2cnn[nH]2)cc1CNc1cc(-c2ccc(F)c(F)c2)ccc1C(=O)OC]

Methyl 3-((5-ethynyl-2-(methoxycarbonyl)benzyl)amino)-3′,4′-difluoro-[1,1′-biphenyl]-4-carboxylate (0.470 g, 1.08 mmol) was dissolved in DMF (12.5 mL) and methanol (1.25 mL). To this solution was added TMS azide (0.426 μL, 3.23 mmol) and CuI (0.041 g, 0.22 mmol) and the mixture was heated at 100° C. in a microwave reactor for 3 h. The cooled reaction mixture was poured into water and the solid was collected by filtration. The crude methyl 3′,4′-difluoro-3-((2-(methoxycarbonyl)-5-(1H-1,2,3-triazol-5-yl)benzyl)amino)-[1,1′-biphenyl]-4-carboxylate (0.52 g) obtained after drying under vacuum was used directly in the next step.

### Step 7.6 Preparation of 3-((2-carboxy-5-(1H-1,2,3-triazol-5-yl)benzyl)amino)-3′,4′-difluoro-[1,1′-biphenyl]-4-carboxylic Acid

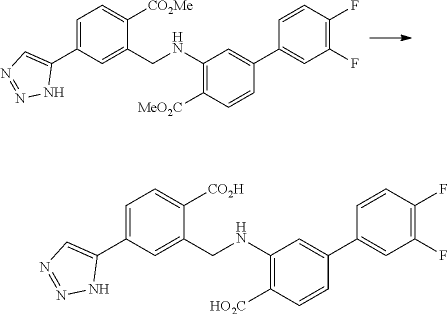 [SMILES: COC(=O)c1ccc(-c2cnn[nH]2)cc1CNc1cc(-c2ccc(F)c(F)c2)ccc1C(=O)OC ; O=C(O)c1ccc(-c2cnn[nH]2)cc1CNc1cc(-c2ccc(F)c(F)c2)ccc1C(=O)O]

Methyl 3′,4′-difluoro-3-((2-(methoxycarbonyl)-5-(1H-1,2,3-triazol-5-yl)benzyl)amino)-[1,1′-biphenyl]-4-carboxylate (0.100 g, crude material) was dissolved in a mixture of methanol (5 mL) and THF (9 mL). Sodium hydroxide (1.25 mL, 2N aqueous) was added and the mixture was heated at 40° C. for 10 h, at which time 2N HCl was added to the cooled solution to adjust the pH to ˜2. Water (30 mL) was added and the volatile organic solvent was eliminated by evaporation. The resulting suspension of solid was filtered and the solid dried under vacuum to afford 3-((2-carboxy-5-(1H-1,2,3-triazol-5-yl)benzyl)amino)-3′,4′-difluoro-[1,1′-biphenyl]-4-carboxylic acid. The solid was subjected to final purification by HPLC.

1H NMR (250 MHz, DEUTERIUM OXIDE) δ ppm 4.93 (br. s., 2H) 6.84 (d, J=8.13 Hz, 1H) 7.07 (s, 1H) 7.46 (t, 2H) 7.63-7.76 (m, 1H) 7.85 (d, J=8.35 Hz, 2H) 7.95-8.04 (m, 1H) 8.23 (s, 1H)

### Example 8: Preparation of N₁-(3-cyano-4-(4-methoxyphenyl)-5-methylthiophen-2-yl)-4-(1H-1,2,3-triazol-4-yl)phthalamide (GO-0000293)

N1-(3-cyano-4-(4-methoxyphenyl)-5-methylthiophen-2-yl)-4-(1H-1,2,3-triazol-4-yl)phthalamide was prepared in several steps.

### Step 8.1 Preparation of 2-(1,3-dioxo-5-(1H-1,2,3-triazol-4-yl)isoindolin-2-yl)-4-(4-methoxyphenyl)-5-methylthiophene-3-carbonitrile

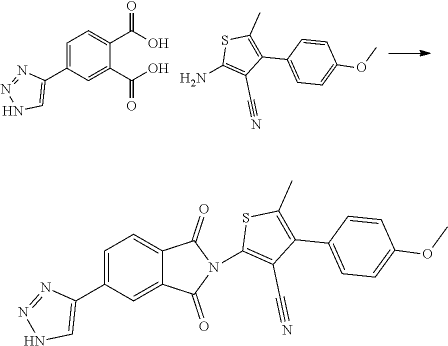 [SMILES: O=C(O)c1ccc(-c2c[nH]nn2)cc1C(=O)O ; COc1ccc(-c2c(C)sc(N)c2C#N)cc1 ; COc1ccc(-c2c(C)sc(N3C(=O)c4ccc(-c5c[nH]nn5)cc4C3=O)c2C#N)cc1]

Using General Procedure #5, 4-(1H-1,2,3-triazol-4-yl)phthalic acid (0.072 g) is was reacted with 2-amino-4-(4-methoxyphenyl)-5-methylthiophene-3-carbonitrile (0.075 g) to afford 2-(1,3-dioxo-5-(1H-1,2,3-triazol-4-yl)isoindolin-2-yl)-4-(4-methoxyphenyl)-5-methylthiophene-3-carbonitrile (0.100 g, 73.8% yield), after drying.

### Step 8.2 Preparation of N1-(3-cyano-4-(4-methoxyphenyl)-5-methylthiophen-2-yl)-4-(1H-1,2,3-triazol-4-yl)phthalamide and N1-(3-cyano-4-(4-methoxyphenyl)-5-methylthiophen-2-yl)-5-(1H-1,2,3-triazol-4-yl)phthalamide

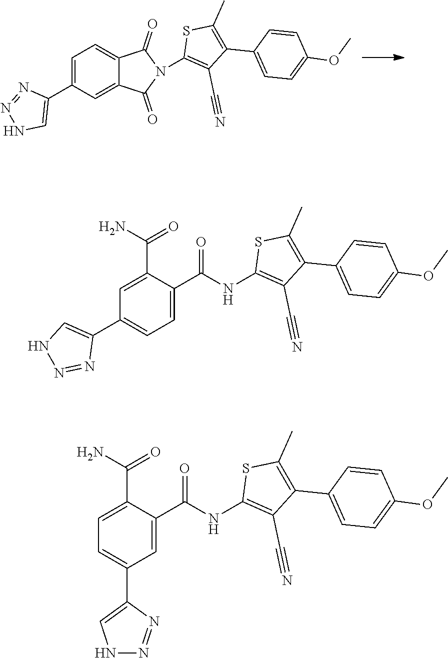 [SMILES: COc1ccc(-c2c(C)sc(N3C(=O)c4ccc(-c5c[nH]nn5)cc4C3=O)c2C#N)cc1 ; COc1ccc(-c2c(C)sc(NC(=O)c3ccc(-c4c[nH]nn4)cc3C(N)=O)c2C#N)cc1 ; COc1ccc(-c2c(C)sc(NC(=O)c3cc(-c4c[nH]nn4)ccc3C(N)=O)c2C#N)cc1]

Using General Procedure #6 for the ring opening of N-aryl phthalimides with ammonia, 2-(1,3-dioxo-5-(1H-1,2,3-triazol-4-yl)isoindolin-2-yl)-4-(4-methoxyphenyl)-5-methylthiophene-3-carbonitrile (0.100 g) was treated with 7M methanolic ammonia (0.33 mL) in THF (6 mL) for 20 min to afford a mixture of N1-(3-cyano-4-(4-methoxyphenyl)-5-methylthiophen-2-yl)-4-(1H-1,2,3-triazol-4-yl)phthalamide and N₁-(3-cyano-4-(4-methoxyphenyl)-5-methylthiophen-2-yl)-5-(1H-1,2,3-triazol-4-yl)phthalamide which were separated and purified by preparative HPLC. The target compound was selected as the isomer, which matched the NMR spectra.

1H NMR (250 MHz, DMSO-d6) δ 8.50 (d, J=13.0 Hz, 1H), 8.26-7.96 (m, 2H), 7.75-7.49 (m, 1H), 7.38 (dd, J=22.6, 8.7 Hz, 1H), 7.17-6.96 (m, 1H), 3.80 (d, J=3.7 Hz, 2H), 3.35 (s, 6H), 2.50 (p, J=1.9 Hz, 4H), 2.35-2.01 (m, 2H).

### Example 9: Preparation of methyl 2-((3-cyano-4-(4-methoxyphenyl)-5-methylthiophen-2-yl)carbamoyl)-5-(1H-1,2,3-triazol-5-yl)benzoate (GO-0000296)

Methyl 2-((3-cyano-4-(4-methoxyphenyl)-5-methylthiophen-2-yl)carbamoyl)-5-(1H-1,2,3-triazol-5-yl)benzoate was prepared in several steps.

### Step 9.1 Preparation of 2-[1,3-dioxo-5-(1H-1,2,3-triazol-5-yl)-2,3-dihydro-1H-isoindol-2-yl]-4-(4-methoxyphenyl)-5-methylthiophene-3-carbonitrile

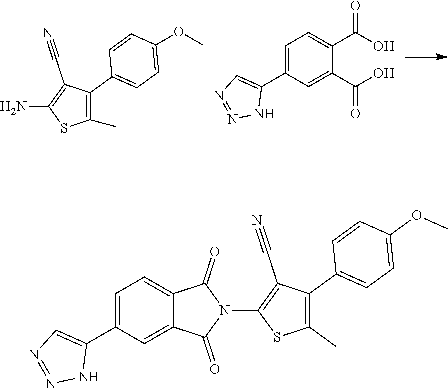 [SMILES: COc1ccc(-c2c(C)sc(N)c2C#N)cc1 ; O=C(O)c1ccc(-c2cnn[nH]2)cc1C(=O)O ; COc1ccc(-c2c(C)sc(N3C(=O)c4ccc(-c5cnn[nH]5)cc4C3=O)c2C#N)cc1]

Using the General Procedure #5 for the reaction of arylamines with phthalic acids to form n-aryl-phthalimides, 2-amino-4-(4-methoxyphenyl)-5-methylthiophene-3-carbonitrile (0.115 g) was reacted with 4-(1H-1,2,3-triazol-5-yl)phthalic acid (0.120 g) in acetic acid (8 mL) for 15 h to afford crude 2-(1,3-dioxo-5-(1H-1,2,3-triazol-5-yl)isoindolin-2-yl)-4-(4-methoxyphenyl)-5-methylthiophene-3-carbonitrile (0.220 g) which was used directly in the next reaction without further purification.

### Step 9.2 Preparation of Methyl 2-((3-cyano-4-(4-methoxyphenyl)-5-methylthiophen-2-yl)carbamoyl)-5-(1H-1,2,3-triazol-5-yl)benzoate (NSQP00529) and methyl 2-((3-cyano-4-(4-methoxyphenyl)-5-methylthiophen-2-yl)carbamoyl)-4-(1H-1,2,3-triazol-5-yl)benzoate

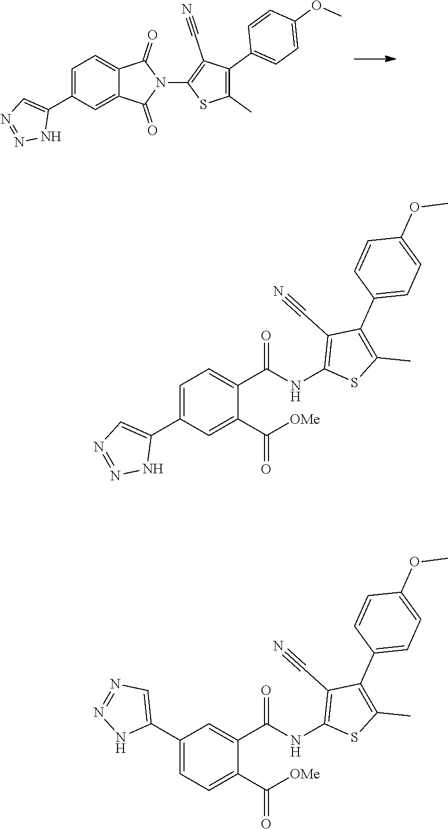 [SMILES: COc1ccc(-c2c(C)sc(N3C(=O)c4ccc(-c5cnn[nH]5)cc4C3=O)c2C#N)cc1 ; COC(=O)c1cc(-c2cnn[nH]2)ccc1C(=O)Nc1sc(C)c(-c2ccc(OC)cc2)c1C#N ; COC(=O)c1ccc(-c2cnn[nH]2)cc1C(=O)Nc1sc(C)c(-c2ccc(OC)cc2)c1C#N]

Using General Procedure 7 for the ring opening of N-aryl phthalimides with sodium methoxide, 2-(1,3-dioxo-5-(1H-1,2,3-triazol-5-yl)isoindolin-2-yl)-4-(4-methoxyphenyl)-5-methylthiophene-3-carbonitrile (0.080 g) was treated with sodium methoxide solution (125 μL) in THF-MeOH (3 mL: 2 mL) for 20 min to afford methyl 2-((3-cyano-4-(4-methoxyphenyl)-5-methylthiophen-2-yl)carbamoyl)-5-(1H-1,2,3-triazol-5-yl)benzoate and methyl 2-((3-cyano-4-(4-methoxyphenyl)-5-methylthiophen-2-yl)carbamoyl)-4-(1H-1,2,3-triazol-5-yl)benzoate after separation on preparative HPLC. The target compound was selected as the isomer, which matched the NMR spectra. 1H NMR (250 MHz, DMSO-d6) δ 12.25 (d, J=16.1 Hz, 1H), 8.45 (d, J=13.7 Hz, 1H), 8.31-7.97 (m, 2H), 7.72 (d, J=8.0 Hz, 1H), 7.34 (d, J=8.7 Hz, 2H), 7.06 (d, J=8.7 Hz, 2H), 3.82 (s, 6H), 2.37-2.12 (m, 3H).

### Example 10: Preparation of (3-carbamoyl-4-((3-cyano-4-(4-methoxyphenyl)-5-methylthiophen-2-yl)carbamoyl)benzoyl)-L-serine (GO-0000305)

(3-carbamoyl-4-((3-cyano-4-(4-methoxyphenyl)-5-methylthiophen-2-yl)carbamoyl)benzoyl)-L-serine was prepared in several steps.

### Step 10.1 Preparation of O-benzyl-N-(2-(3-cyano-4-(4-methoxyphenyl)-5-methylthiophen-2-yl)-1,3-dioxoisoindoline-5-carbonyl)-L-serine

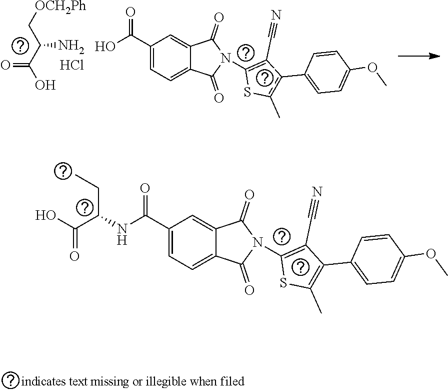

Using General Procedure #8 for the amidation of N-arylphthalimido-5-carboxylic acids, O-benzyl-L-serine hydrochloride (0.070 g) was reacted with 2-(3-cyano-4-(4-methoxyphenyl)-5-methylthiophen-2-yl)-1,3-dioxoisoindoline-5-carboxylic acid (0.120 g), HATU (0.115 g) and DIEA (125 μL) in DMF (4 mL) for 4.5 h to afford O-benzyl-N-(2-(3-cyano-4-(4-methoxyphenyl)-5-methylthiophen-2-yl)-1,3-dioxoisoindoline-5-carbonyl)-L-serine (0.163 g, 95% yield).

### Step 10.2 Preparation of (2-(3-cyano-4-(4-methoxyphenyl)-5-methylthiophen-2-yl)-1,3-dioxoisoindoline-5-carbonyl)-L-serine

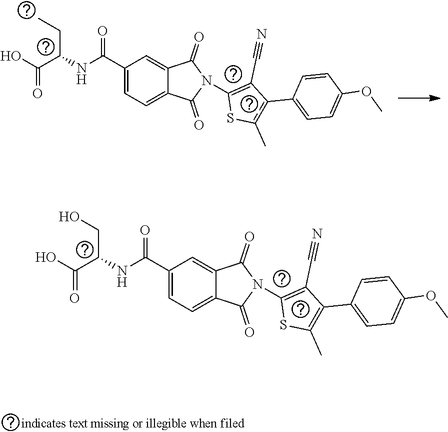 [SMILES: CC[C@H](NC(=O)c1ccc2c(c1)C(=O)N(c1sc(C)c(-c3ccc(OC)cc3)c1C#N)C2=O)C(=O)O ; COc1ccc(-c2c(C)sc(N3C(=O)c4ccc(C(=O)N[C@@H](CO)C(=O)O)cc4C3=O)c2C#N)cc1]

O-benzyl-N-(2-(3-cyano-4-(4-methoxyphenyl)-5-methylthiophen-2-yl)-1,3-dioxoisoindoline-5-carbonyl)-L-serine (0.163 g) was dissolved in a mixture of THF-MeOH (1:1, 15 mL) and treated at 1 atms with hydrogen gas and Pd/C (0.080 g, 10%) for 2.5 h. The reaction mixture was filtered through a pad of Celite to remove the catalyst, rinsing the filter cake with THF and the filtered solution was concentrated to dryness and dried in vacuo to afford crude (2-(3-cyano-4-(4-methoxyphenyl)-5-methylthiophen-2-yl)-1,3-dioxoisoindoline-5-carbonyl)-L-serine (0.160 g) which was used in the next step without further purification.

### Step 10.3 Preparation of (3-carbamoyl-4-((3-cyano-4-(4-methoxyphenyl)-5-methylthiophen-2-yl)carbamoyl)benzoyl)-L-serine (NSQP00539) and (4-carbamoyl-3-((3-cyano-4-(4-methoxyphenyl)-5-methylthiophen-2-yl)carbamoyl)benzoyl)-L-serine

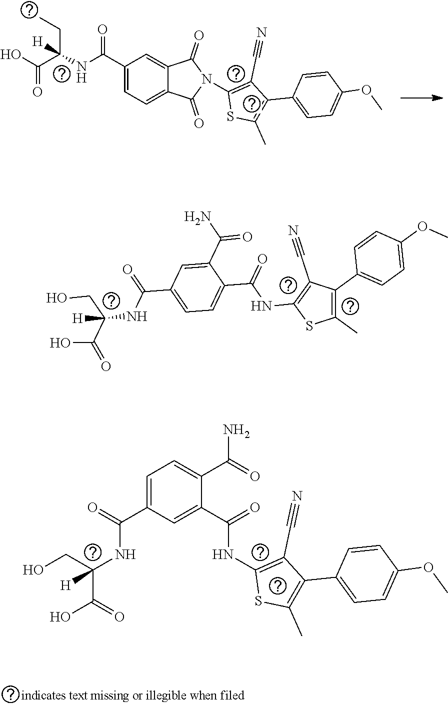

Using General Procedure #6 for the ring opening of N-aryl phthalimides with ammonia, (2-(3-cyano-4-(4-methoxyphenyl)-5-methylthiophen-2-yl)-1,3-dioxoisoindoline-5-carbonyl)-L-serine (0.080 g) was treated with methanolic ammonia (7M, 0.175 mL) for 40 min in THF (6 mL) to afford, after HPLC separation and purification, (3-carbamoyl-4-((3-cyano-4-(4-methoxyphenyl)-5-methylthiophen-2-yl)carbamoyl)benzoyl)-L-serine (NSQP00539) and (4-carbamoyl-3-((3-cyano-4-(4-methoxyphenyl)-5-methylthiophen-2-yl)carbamoyl)benzoyl)-L-serine. The target compound was selected as the isomer, which matched the NMR spectra.

### Example 11: Preparation of 3-carbamoyl-4-[(3-cyano-4,5-diphenylthiophen-2-yl)carbamoyl]benzoic acid (GO-0000311)

3-carbamoyl-4-[(3-cyano-4,5-diphenylthiophen-2-yl)carbamoyl]benzoic acid was prepared in several steps.

### Step 11.1 Preparation of 2-(3-cyano-4,5-diphenylthiophen-2-yl)-1,3-dioxoisoindoline-5-carboxylic acid

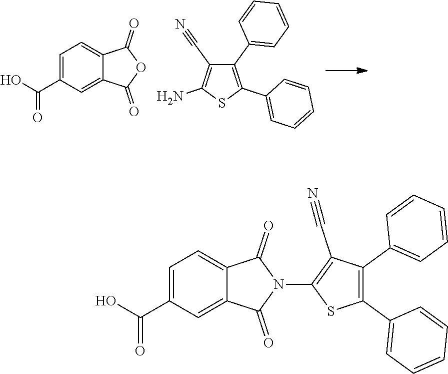 [SMILES: O=C(O)c1ccc2c(c1)C(=O)OC2=O ; N#Cc1c(N)sc(-c2ccccc2)c1-c1ccccc1 ; N#Cc1c(N2C(=O)c3ccc(C(=O)O)cc3C2=O)sc(-c2ccccc2)c1-c1ccccc1]

Using General Procedure #5 for the reaction of arylamines with phthalic acids to form N-aryl-phthalimides, 1,3-dioxo-1,3-dihydroisobenzofuran-5-carboxylic acid (0.300 g) and 2-amino-4,5-diphenylthiophene-3-carbonitrile (0.431 g) were reacted in acetic acid (18 mL) for 17 h to afford 2-(3-cyano-4,5-diphenylthiophen-2-yl)-1,3-dioxoisoindoline-5-carboxylic acid (0.698 g) which was used without purification in the next step.

### Step 11.2 Preparation of 4-carbamoyl-3-((3-cyano-4,5-diphenylthiophen-2-yl)carbamoyl)benzoic acid and 3-carbamoyl-4-((3-cyano-4,5-diphenylthiophen-2-yl)carbamoyl)benzoic acid

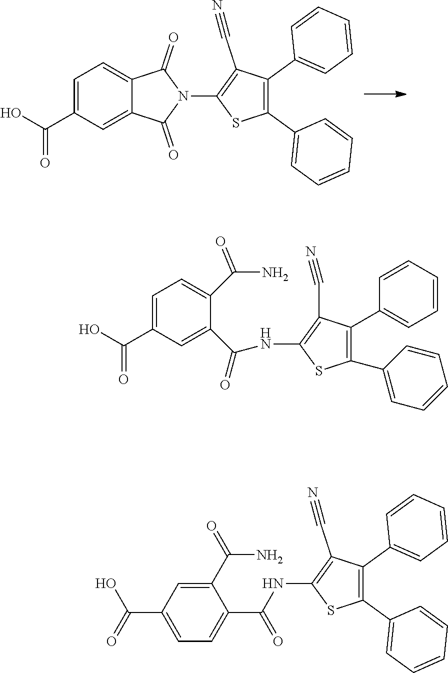 [SMILES: N#Cc1c(N2C(=O)c3ccc(C(=O)O)cc3C2=O)sc(-c2ccccc2)c1-c1ccccc1 ; N#Cc1c(NC(=O)c2cc(C(=O)O)ccc2C(N)=O)sc(-c2ccccc2)c1-c1ccccc1 ; N#Cc1c(NC(=O)c2ccc(C(=O)O)cc2C(N)=O)sc(-c2ccccc2)c1-c1ccccc1]

Using General Procedure #6 for the ring opening of N-aryl phthalimides with ammonia, 2-(3-cyano-4,5-diphenylthiophen-2-yl)-1,3-dioxoisoindoline-5-carboxylic acid (0.100 g) was reacted with methanolic ammonia (0.32 mL) in THF (6 mL) for 20 min to afford a mixture of 4-carbamoyl-3-((3-cyano-4,5-diphenylthiophen-2-yl)carbamoyl)benzoic acid (NSQP00545) and 3-carbamoyl-4-((3-cyano-4,5-diphenylthiophen-2-yl)carbamoyl)benzoic acid which was separated into its pure components by preparative HPLC. The target compound was selected as the isomer, which matched the NMR spectra. 1H NMR (250 MHz, DMSO-d6) δ 8.54-8.35 (m, 1H), 8.26 (s, 1H), 8.21-8.03 (m, 1H), 7.73 (d, J=8.0 Hz, 1H), 7.54 (s, 1H), 7.40 (dt, J=12.3, 3.7 Hz, 4H), 7.28 (dd, J=7.8, 2.8 Hz, 4H), 7.18 (dd, J=7.3, 2.6 Hz, 2H).

### Example 12: 4-((3-cyano-4-(4-methoxyphenyl)-5-methylthiophen-2-yl)carbamoyl)-3-(hydroxymethyl)benzoic acid (GO-0000312)

4-((3-cyano-4-(4-methoxyphenyl)-5-methylthiophen-2-yl)carbamoyl)-3-(hydroxymethyl)benzoic acid was prepared in several steps.

### Step 12.1 Preparation of 2-(3-cyano-4-(4-methoxyphenyl)-5-methylthiophen-2-yl)-1,3-dioxoisoindoline-5-carboxylic acid

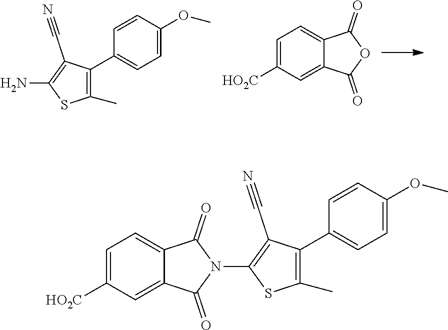 [SMILES: COc1ccc(-c2c(C)sc(N)c2C#N)cc1 ; O=C(O)c1ccc2c(c1)C(=O)OC2=O ; COc1ccc(-c2c(C)sc(N3C(=O)c4ccc(C(=O)O)cc4C3=O)c2C#N)cc1]

2-amino-4-(4-methoxyphenyl)-5-methylthiophene-3-carbonitrile (2 g, 8.18 mmol) and 1,3-dioxo-1,3-dihydroisobenzofuran-5-carboxylic acid (1.58 g, 8.18 mmol) were dissolved in acetic acid (80 mL) and heated for 23 h at 120° C. Upon cooling, the product precipitated from solution and was collected by filtration, rinsed with water, and dried under high vacuum to afford pure 2-(3-cyano-4-(4-methoxyphenyl)-5-methylthiophen-2-yl)-1,3-dioxoisoindoline-5-carboxylic acid (2.39 g, 70% yield).

### Step 12.2 Preparation of 4-((3-cyano-4-(4-methoxyphenyl)-5-methylthiophen-2-yl)carbamoyl)-3-(hydroxymethyl)benzoic acid and 3-((3-cyano-4-(4-methoxyphenyl)-5-methylthiophen-2-yl)carbamoyl)-4-(hydroxymethyl)benzoic acid 3-((3-cyano-4-(4-methoxyphenyl)-5-methylthiophen-2-yl)carbamoyl)-4-(hydroxymethyl)benzoic acid

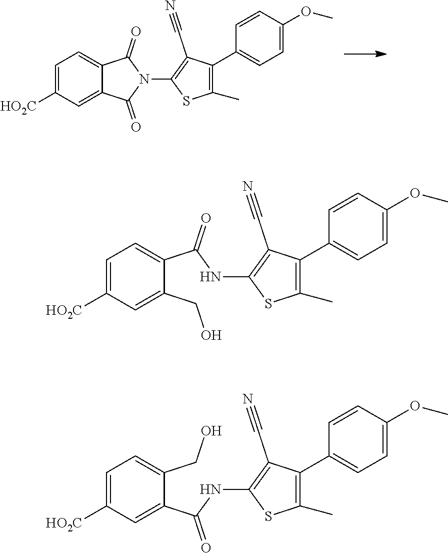 [SMILES: COc1ccc(-c2c(C)sc(N3C(=O)c4ccc(C(=O)O)cc4C3=O)c2C#N)cc1 ; COc1ccc(-c2c(C)sc(NC(=O)c3ccc(C(=O)O)cc3CO)c2C#N)cc1 ; COc1ccc(-c2c(C)sc(NC(=O)c3cc(C(=O)O)ccc3CO)c2C#N)cc1]

2-(3-cyano-4-(4-methoxyphenyl)-5-methylthiophen-2-yl)-1,3-dioxoisoindoline-5-carboxylic acid (0.075 g, 0.18 mmol) was dissolved in a mixture of THF (3 mL) and MeOH (1 mL) and treated with sodium borohydride (0.014 g, 0.36 mmol) at room temperature. After stirring for 10 min, the reaction was quenched by the addition of saturated NH₄Cl solution at room temperature, and then poured into water. The solution was acidified to pH-2 by the addition of HCl (1 N) and was extracted with ethyl acetate (3×). The combined organic layers were washed with water, brine, dried over sodium sulfate, filtered and concentrated and dried under high vacuum. Preparative HPLC afforded pure samples of 4-((3-cyano-4-(4-methoxyphenyl)-5-methylthiophen-2-yl)carbamoyl)-3-(hydroxymethyl)benzoic acid and 3-((3-cyano-4-(4-methoxyphenyl)-5-methylthiophen-2-yl)carbamoyl)-4-(hydroxymethyl)benzoic acid 3-((3-cyano-4-(4-methoxyphenyl)-5-methylthiophen-2-yl)carbamoyl)-4-(hydroxymethyl)benzoic acid. The target compound was selected as the isomer, which matched the NMR spectra.

### Example 14: Preparation of 4-{[3-cyano-4-(4-methoxyphenyl)-5-methylthiophen-2-yl]carbamoyl}-3-{[2-(dimethylamino)ethyl]carbamoyl}benzoic acid (GO-0000319)

4-{[3-cyano-4-(4-methoxyphenyl)-5-methylthiophen-2-yl]carbamoyl}-3-{[2-(dimethylamino)ethyl]carbamoyl}benzoic acid was prepared in several steps.

### Step 14.1 Preparation of 2-(3-cyano-4-(4-methoxyphenyl)-5-methylthiophen-2-yl)-1,3-dioxoisoindoline-5-carboxylic acid

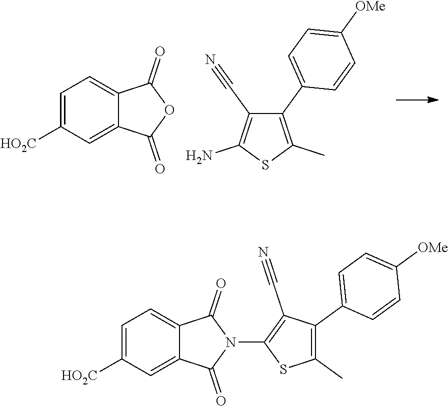 [SMILES: O=C(O)c1ccc2c(c1)C(=O)OC2=O ; COc1ccc(-c2c(C)sc(N)c2C#N)cc1 ; COc1ccc(-c2c(C)sc(N3C(=O)c4ccc(C(=O)O)cc4C3=O)c2C#N)cc1]

Using General Procedure #9, 1,3-dioxo-1,3-dihydroisobenzofuran-5-carboxylic acid (0.394 g) was reacted with 2-amino-4-(4-methoxyphenyl)-5-methylthiophene-3-carbonitrile (0.500 g) were reacted to afford 2-(3-cyano-4-(4-methoxyphenyl)-5-methylthiophen-2-yl)-1,3-dioxoisoindoline-5-carboxylic acid (0.600 g, 70% yield).

### Step 14.2 Preparation of 4-((3-cyano-4-(4-methoxyphenyl)-5-methylthiophen-2-yl)carbamoyl)-3-((2-(dimethylamino)ethyl)carbamoyl)benzoic acid and 3-((3-cyano-4-(4-methoxyphenyl)-5-methylthiophen-2-yl)carbamoyl)-4-((2-(dimethylamino)ethyl)carbamoyl)benzoic acid

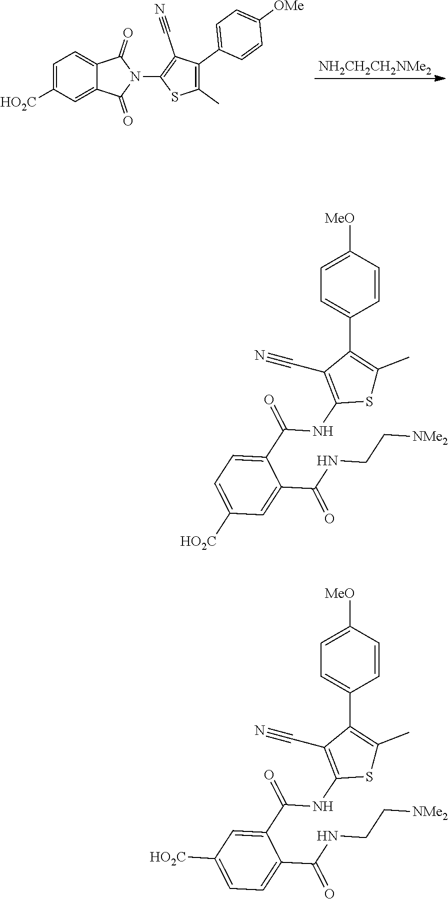

Using General Procedure #6, 2-(3-cyano-4-(4-methoxyphenyl)-5-methylthiophen-2-yl)-1,3-dioxoiso-indoline-5-carboxylic acid (0.100 g, 1 equiv) was reacted with N¹,N¹-dimethylethane-1,2-diamine (78.3 μL, 3 equiv) in THF (4 mL) to afford a mixture of 4-((3-cyano-4-(4-methoxyphenyl)-5-methylthiophen-2-yl)carbamoyl)-3-((2-(dimethylamino)ethyl)carbamoyl)benzoic acid and 3-((3-cyano-4-(4-methoxyphenyl)-5-methylthiophen-2-yl)carbamoyl)-4-((2-(dimethylamino)ethyl)carbamoyl)benzoic acid, which were separated and purified by HPLC. The target compound was selected as the isomer, which matched the NMR spectra. 1H NMR (250 MHz, DMSO-d6) δ 8.45 (s, 1H), 7.70 (d, J=19.8 Hz, 3H), 7.50 (dd, J=16.8, 7.8 Hz, 2H), 7.35 (d, J=8.3 Hz, 2H), 7.29-7.21 (m, 2H), 7.12-6.92 (m, 5H), 3.92-3.84 (m, 2H), 3.80 (d, J=6.6 Hz, 5H), 3.59 (s, 3H), 2.85 (s, 4H), 2.70 (s, 6H), 2.33-2.23 (m, 3H), 2.11 (d, J=1.2 Hz, 3H).

### Example 15: Preparation of N1-(3-cyano-4-(4-methoxyphenyl)-5-methylthiophen-2-yl)-4-(hydroxymethyl)-N2-methylphthalamide (GO-0000329)

N1-(3-cyano-4-(4-methoxyphenyl)-5-methylthiophen-2-yl)-4-(hydroxymethyl)-N2-methylphthalamide was prepared in several steps.

### Step 15.1 Preparation of 2-(5-(hydroxymethyl)-1,3-dioxoisoindolin-2-yl)-4-(4-methoxyphenyl)-5-methylthiophene-3-carbonitrile

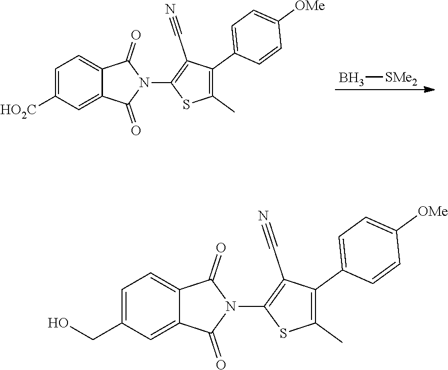

2-(3-cyano-4-(4-methoxyphenyl)-5-methylthiophen-2-yl)-1,3-dioxoisoindoline-5-carboxylic acid (0.400 g, 0.955 mmol) was dissolved in THF (9 mL) and treated with borane-methylsulfide complex ((0.96 mL, 2 M in THF, 2 equiv) at 0° C. and then allowed to rise to room temperature and stirred overnight. Saturated ammonium chloride (10 mL) was added to quench the reaction which was then poured into water and extracted with ethyl acetate (3×), dried over sodium sulfate, concentrated and dried under vacuum to afford a crude product (0.436 g) which was used without further purification in the following step.

### Step 15.2 Preparation of of N1-(3-cyano-4-(4-methoxyphenyl)-5-methylthiophen-2-yl)-4-(hydroxymethyl)-N2-methylphthalamide and 2-(3-cyano-4-(4-methoxyphenyl)-5-methylthiophen-2-yl)-1,3-dioxoisoindoline-5-carboxylic Acid

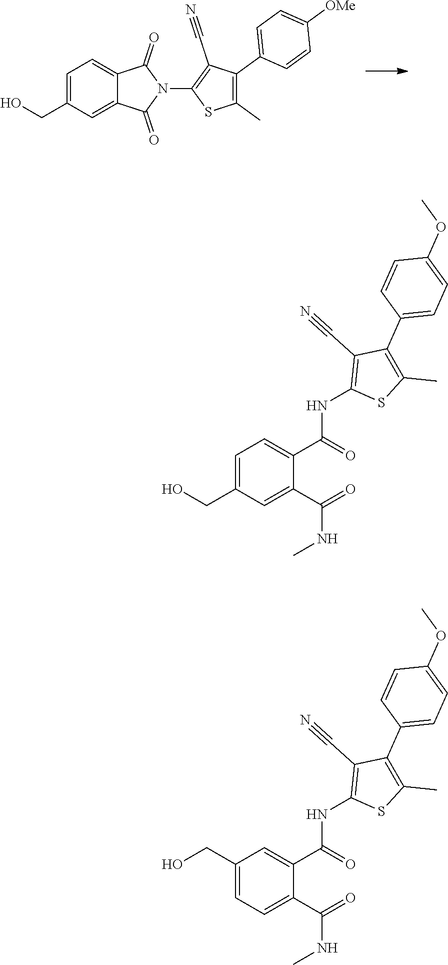 [SMILES: COc1ccc(-c2c(C)sc(N3C(=O)c4ccc(CO)cc4C3=O)c2C#N)cc1 ; CNC(=O)c1cc(CO)ccc1C(=O)Nc1sc(C)c(-c2ccc(OC)cc2)c1C#N ; CNC(=O)c1ccc(CO)cc1C(=O)Nc1sc(C)c(-c2ccc(OC)cc2)c1C#N]

Using General Procedure #6, 2-(3-cyano-4-(4-methoxyphenyl)-5-methylthiophen-2-yl)-1,3-dioxo-isoindoline-5-carboxylic acid (0.065 g) was reacted in THF (3 mL) with methylamine (0.4 mL, 2M in THF), to afford N1-(3-cyano-4-(4-methoxyphenyl)-5-methylthiophen-2-yl)-4-(hydroxymethyl)-N2-methylphthalamide (NSQP00564) and 2-(3-cyano-4-(4-methoxyphenyl)-5-methylthiophen-2-yl)-1,3-dioxoisoindoline-5-carboxylic acid after separation and purification by preparative HPLC. The target compound was selected as the isomer, which matched the NMR spectra. 1H NMR (250 MHz, DMSO-d6) δ 12.11 (d, J=8.0 Hz, 1H), 8.47 (d, J=5.3 Hz, 1H), 7.70-7.43 (m, 3H), 7.39-7.25 (m, 2H), 7.06 (d, J=8.7 Hz, 2H), 5.45 (t, J=5.5 Hz, 1H), 4.60 (d, J=5.7 Hz, 2H), 3.81 (d, J=0.6 Hz, 3H), 2.74 (d, J=4.5 Hz, 3H), 2.29 (s, 3H).

### Example 16: 4-({4-carboxy-3′,4′-difluoro-[1,1′-biphenyl]-3-yl}carbamoyl)-6-hydroxybenzene-1,3-dicarboxylic acid (GO-0001177)

4-({4-carboxy-3′,4′-difluoro-[1,1′-biphenyl]-3-yl}carbamoyl)-6-hydroxybenzene-1,3-dicarboxylic acid was prepared in one step.

### Step 16.1 Preparation of 4-({4-carboxy-3′,4′-difluoro-[1,1′-biphenyl]-3-yl}carbamoyl)-6-hydroxybenzene-1,3-dicarboxylic acid

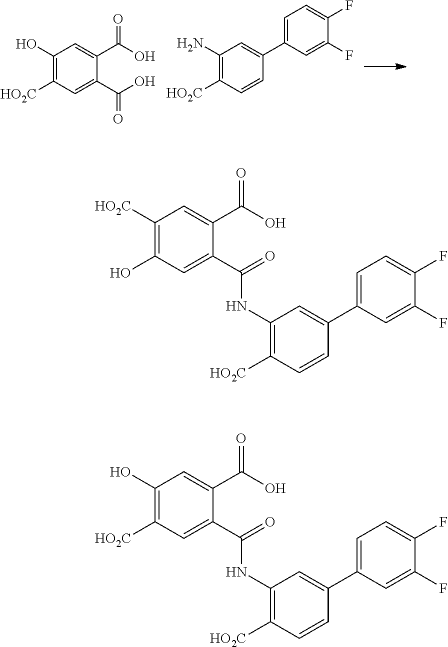 [SMILES: O=C(O)c1cc(C(=O)O)c(C(=O)O)cc1O ; Nc1cc(-c2ccc(F)c(F)c2)ccc1C(=O)O ; O=C(O)c1cc(C(=O)O)c(C(=O)Nc2cc(-c3ccc(F)c(F)c3)ccc2C(=O)O)cc1O ; O=C(O)c1cc(C(=O)Nc2cc(-c3ccc(F)c(F)c3)ccc2C(=O)O)c(C(=O)O)cc1O]

A mixture of 5-hydroxybenzene-1,2,4-tricarboxylic acid (0.036 g, 0.16 mmol) and 3-amino-3′,4′-difluoro-[1,1′-biphenyl]-4-carboxylic acid (0.040 g, 0.16 mmol) dissolved in isobutyric acid (3 mL) was heated in a microwave reactor at 140° C. for 20 min. The cooled reaction mixture was poured into water (40 mL) and the precipitate was filtered and dried under high vacuum. Separation by preparative HPLC afforded pure 4-((4-carboxy-3′,4′-difluoro-[1,1′-biphenyl]-3-yl)carbamoyl)-6-hydroxyisophthalic acid (major) and 2-((4-carboxy-3′,4′-difluoro-[1,1′-biphenyl]-3-yl)carbamoyl)-5-hydroxyterephthalic acid (minor). After preparative HPLC the target compound was selected as the isomer, which didn't match the NMR spectra of undesirable isomer.

### Example 17: Preparation of 4-([1,1′-biphenyl]-3-carboxamido)isophthalic acid (GO-0001181)

4-([1,1′-biphenyl]-3-carboxamido)isophthalic acid was prepared in several steps.

### Step 17.1 Preparation of [1,1′-biphenyl]-3-carbonyl chloride

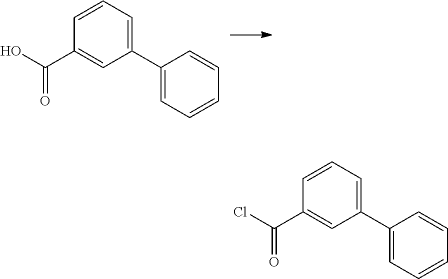 [SMILES: O=C(O)c1cccc(-c2ccccc2)c1 ; O=C(Cl)c1cccc(-c2ccccc2)c1]

[1,1′-biphenyl]-3-carboxylic acid (0.100 g, 0.504 mmol) was treated with thionyl chloride (3.5 mL) at 90° C. for 3.5 h with stirring. The cooled solution was then evaporated to dryness and thoroughly dried under high vacuum to afford the crude acid chloride, [1,1′-biphenyl]-3-carbonyl chloride Step 17.2 Preparation of diethyl 4-([1,1′-biphenyl]-3-carboxamido)isophthalate

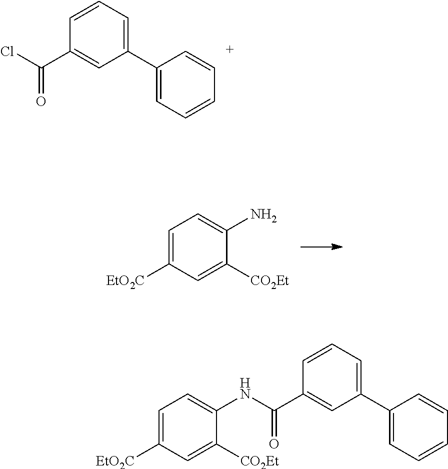 [SMILES: O=C(Cl)c1cccc(-c2ccccc2)c1 ; CCOC(=O)c1ccc(N)c(C(=O)OCC)c1 ; CCOC(=O)c1ccc(NC(=O)c2cccc(-c3ccccc3)c2)c(C(=O)OCC)c1]

The crude [1,1′-biphenyl]-3-carbonyl chloride from the previous step was dissolved in THF (5 mL) and treated with diethyl 4-aminoisophthalate (0.120 mL, 0.505 mmol) at room temperature for 2 days. The solvent was evaporated to dryness and the crude product dried thoroughly under vacuum.

### Step 17.3 Preparation of 4-([1,1′-biphenyl]-3-carboxamido)isophthalic acid

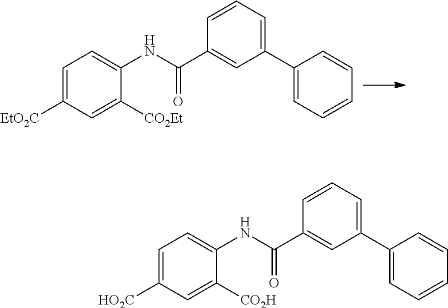 [SMILES: CCOC(=O)c1ccc(NC(=O)c2cccc(-c3ccccc3)c2)c(C(=O)OCC)c1 ; O=C(O)c1ccc(NC(=O)c2cccc(-c3ccccc3)c2)c(C(=O)O)c1]

The crude diester from the previous step by hydrolysis by sodium hydroxide (1.26 mL, 2M aqueous, 5 equiv)) at room temperature, stirring in a mixture of methanol (6 mL) and THF (3 mL) overnight. Acidification with HCl (0.2N) to pH-3 was followed by evaporation of the solution to dryness. After drying under vacuum, the product was purified by preparative HPLC to afford 4-([1,1′-biphenyl]-3-carboxamido)isophthalic acid. 1H NMR (250 MHz, DMSO-d6) δ 8.87 (d, J=8.8 Hz, 1H), 8.64 (d, J=2.2 Hz, 1H), 8.29-8.15 (m, 2H), 8.03-7.93 (m, 2H), 7.83-7.65 (m, 3H), 7.60-7.37 (m, 3H).

### Example 18: Preparation of 2-([1,1′-biphenyl]-3-carboxamido)-4-chloro-5-(1H-1,2,3-triazol-5-yl)benzoic acid (GO-0001321)

2-([1,1′-biphenyl]-3-carboxamido)-4-chloro-5-(1H-1,2,3-triazol-5-yl)benzoic acid was prepared in several steps.

### Step 18.1 Preparation of methyl 2-amino-5-bromo-4-chlorobenzoate

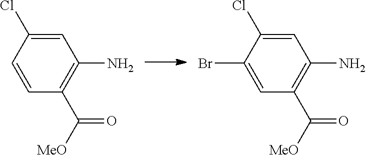 [SMILES: COC(=O)c1ccc(Cl)cc1N ; COC(=O)c1cc(Br)c(Cl)cc1N]

Methyl 2-amino-4-chlorobenzoate (1 g, 5.38 mmol) was dissolved in DMF (10 mL) and treated at room temperature with N-bromosuccinimide ((0.960 g, 5.38 mmol), stirring for 1.5 h. The reaction mixture was poured into ice water and extracted with ethyl acetate (3×). The combined organic layers were washed with water followed by brine, dried over sodium sulfate, filtered and concentrated. The resulting solid was washed with a mixture of ether-hexane (1 mL-5 mL) and dried under vacuum to afford pure methyl 2-amino-5-bromo-4-chlorobenzoate.

### Step 18.2 Preparation of methyl 2-([1,1′-biphenyl]-3-carboxamido)-5-bromo-4-chlorobenzoate

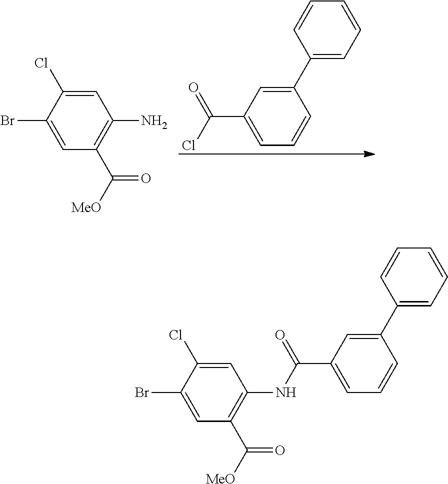 [SMILES: COC(=O)c1cc(Br)c(Cl)cc1N ; O=C(Cl)c1cccc(-c2ccccc2)c1 ; COC(=O)c1cc(Br)c(Cl)cc1NC(=O)c1cccc(-c2ccccc2)c1]

Using General Procedure 10, methyl 2-amino-5-bromo-4-chlorobenzoate (0.334 g, 1.26 mmol) was reacted with [1,1′-biphenyl]-3-carbonyl chloride to afford methyl 2-([1,1′-biphenyl]-3-carboxamido)-5-bromo-4-chlorobenzoate (0.398 g, 91%).

### Step 18.3 Preparation of methyl 2-([1,1′-biphenyl]-3-carboxamido)-4-chloro-5-((trimethylsilyl)ethynyl)benzoate and methyl 2-([1,1′-biphenyl]-3-carboxamido)-4-chloro-5-ethynylbenzoate

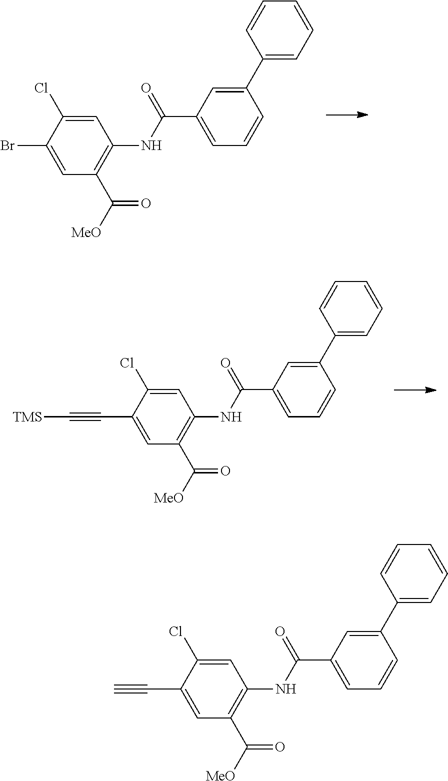 [SMILES: COC(=O)c1cc(Br)c(Cl)cc1NC(=O)c1cccc(-c2ccccc2)c1 ; COC(=O)c1cc(C#C[Si](C)(C)C)c(Cl)cc1NC(=O)c1cccc(-c2ccccc2)c1 ; C#Cc1cc(C(=O)OC)c(NC(=O)c2cccc(-c3ccccc3)c2)cc1Cl]

Using General Procedure 3, methyl 2-([1,1′-biphenyl]-3-carboxamido)-5-bromo-4-chlorobenzoate (0.265 g) was TMS-ethynylated to afford methyl 2-([1,1′-biphenyl]-3-carboxamido)-4-chloro-5-((trimethylsilyl)-ethynyl)benzoate (0.247 g). The TMS protecting was removed according to the General Procedure, on a 0.213 g scale, to afford pure methyl 2-([1,1′-biphenyl]-3-carboxamido)-4-chloro-5-ethynylbenzoate (0.146 g, 74% yield)

### Step 18.4 Preparation of 2-([1,1′-biphenyl]-3-carboxamido)-4-chloro-5-(1H-1,2,3-triazol-5-yl)benzoic acid

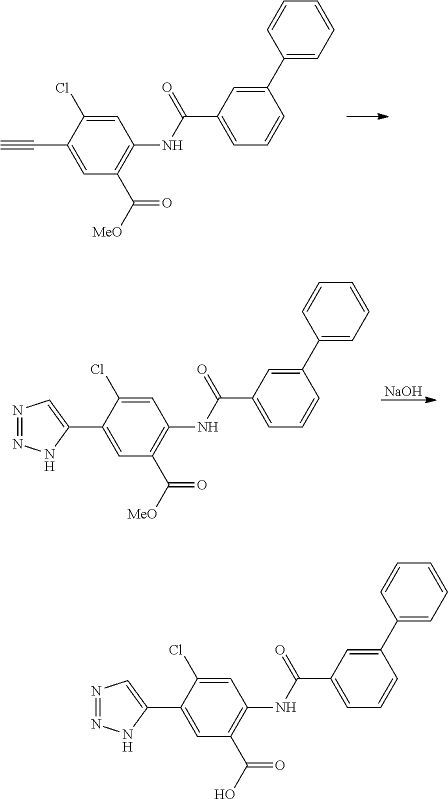 [SMILES: C#Cc1cc(C(=O)OC)c(NC(=O)c2cccc(-c3ccccc3)c2)cc1Cl ; COC(=O)c1cc(-c2cnn[nH]2)c(Cl)cc1NC(=O)c1cccc(-c2ccccc2)c1 ; "NaOH" ; O=C(Nc1cc(Cl)c(-c2cnn[nH]2)cc1C(=O)O)c1cccc(-c2ccccc2)c1]

Using General Procedure 4, methyl 2-([1,1′-biphenyl]-3-carboxamido)-4-chloro-5-ethynylbenzoate (0.104 g) was reacted with TMS-azide to afford methyl 2-([1,1′-biphenyl]-3-carboxamido)-4-chloro-5-(1H-1,2,3-triazol-5-yl)benzoate which was hydrolyzed to the corresponding carboxylic acid, 2-([1,1′-biphenyl]-3-carboxamido)-4-chloro-5-(1H-1,2,3-triazol-5-yl)benzoic acid, according to the General Procedure, and purified by preparative HPLC.

### Example 19: Preparation of 3-((2-carboxy-5-chloro-4-(1H-1,2,3-triazol-5-yl)phenyl)carbamoyl)-3′,4′-difluoro-[1,1′-biphenyl]-4-carboxylic acid (GO-0001330)

3-((2-carboxy-5-chloro-4-(1H-1,2,3-triazol-5-yl)phenyl)carbamoyl)-3′,4′-difluoro-[1,1′-biphenyl]-4-carboxylic acid was prepared in several steps.

### Step 19.1 Preparation of 5-bromo-2-(ethoxycarbonyl)benzoic acid and 4-bromo-2-(ethoxycarbonyl)benzoic acid

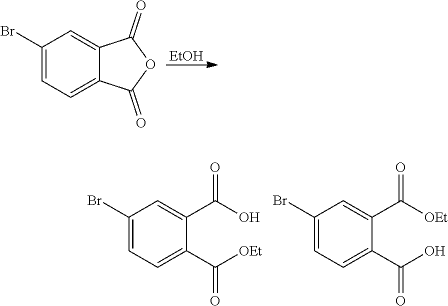

5-bromoisobenzofuran-1,3-dione (2 g) was dissolved in ethanol (17 mL) and heated in a microwave reactor at 90° C. for 1 h. The solvent was evaporated to dryness, the residue was dried under vacuum and separated and purified by preparative HPLC to afford 5-bromo-2-(ethoxycarbonyl)benzoic acid (1.427 g, 30%) as well as the isomer 4-bromo-2-(ethoxycarbonyl)benzoic acid which was not used.

### Step 19.2 Preparation of 4-(ethoxycarbonyl)-3′,4′-difluoro-[1,1′-biphenyl]-3-carboxylic Acid

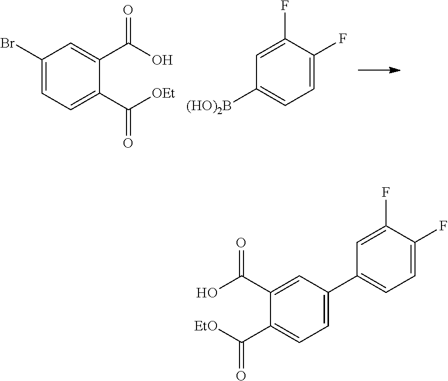 [SMILES: CCOC(=O)c1ccc(Br)cc1C(=O)O ; OB(O)c1ccc(F)c(F)c1 ; CCOC(=O)c1ccc(-c2ccc(F)c(F)c2)cc1C(=O)O]

Using General Procedure 1, 5-bromo-2-(ethoxycarbonyl)benzoic acid (0.310 g) was coupled with (3,4-difluorophenyl)boronic acid (0.180 g) to afford pure 4-(ethoxycarbonyl)-3′,4′-difluoro-[1,1′-biphenyl]-3-carboxylic acid (0.296 g, 85%).

### Step 19.3 Preparation of ethyl 3-((4-bromo-5-chloro-2-(methoxycarbonyl)phenyl)carbamoyl)-3′,4′-difluoro-[1,1′-biphenyl]-4-carboxylate

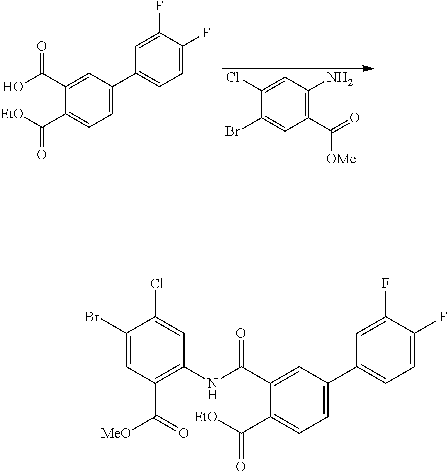 [SMILES: CCOC(=O)c1ccc(-c2ccc(F)c(F)c2)cc1C(=O)O ; COC(=O)c1cc(Br)c(Cl)cc1N ; CCOC(=O)c1ccc(-c2ccc(F)c(F)c2)cc1C(=O)Nc1cc(Cl)c(Br)cc1C(=O)OC]

4-(ethoxycarbonyl)-3′,4′-difluoro-[1,1′-biphenyl]-3-carboxylic acid (0.135 g, 0.44 mmol) and methyl 2-amino-5-bromo-4-chlorobenzoate (0.116 g, 0.44 mmol) were dissolved in pyridine (4.4 mL) and treated with POCl₃ (0.82 μL). at 0° C. for 0.5 h. The reaction mixture was quenched by the addition of saturated NaHCO₃ at 0° C. The mixture was poured into water, extracted with ethyl acetate (3×), the combined organic layers dried over sodium sulfate, filtered, concentrated and then purified by flash chromatography (SiO₂, hexane-ethyl acetate 0 to 40%) to afford pure ethyl 3-((4-bromo-5-chloro-2-(methoxy-carbonyl)phenyl)carbamoyl)-3′,4′-difluoro-[1,1′-biphenyl]-4-carboxylate (0.183 g, 78% yield). Hydrolysis of the esters according to the second half of General Procedure 4, afforded the diacid, 3-((2-carboxy-5-chloro-4-(1H-1,2,3-triazol-5-yl)phenyl)carbamoyl)-3′,4′-difluoro-[1,1′-biphenyl]-4-carboxylic acid after purification by preparative HPLC. The target compound was selected as the isomer, which didn't match the NMR spectra of undesirable isomer.

### Example 20: 3-((2-carboxy-4-chloro-5-(1H-1,2,3-triazol-5-yl)phenyl)carbamoyl)-3′,4′-difluoro-[1,1′-biphenyl]-4-carboxylic acid (GO-0001331)

3-((2-carboxy-4-chloro-5-(1H-1,2,3-triazol-5-yl)phenyl)carbamoyl)-3′,4′-difluoro-[1,1′-biphenyl]-4-carboxylic acid was prepared in several steps.

### Step 20.1 Preparation of methyl 2-amino-4-bromo-5-chlorobenzoate

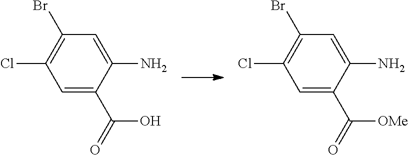 [SMILES: Nc1cc(Br)c(Cl)cc1C(=O)O ; COC(=O)c1cc(Cl)c(Br)cc1N]

2-amino-4-bromo-5-chlorobenzoic acid(0.500 g, 2 mmol) was treated with TMS-diazomethane (1.2 mL, 2M in ethyl ether) in a mixture of ethyl ether-methanol (20 mL-2 mL) at 0° C. for 0.5 h followed by room temperature for 1.5 h. The solvent was evaporated, the residue thoroughly dried to afford methyl 2-amino-4-bromo-5-chlorobenzoate(0.502 g) which was used in the following step.

### Step 20.2 Preparation of ethyl 3-((5-bromo-4-chloro-2-(methoxycarbonyl)phenyl)carbamoyl)-3′,4′-difluoro-[1,1′-biphenyl]-4-carboxylate

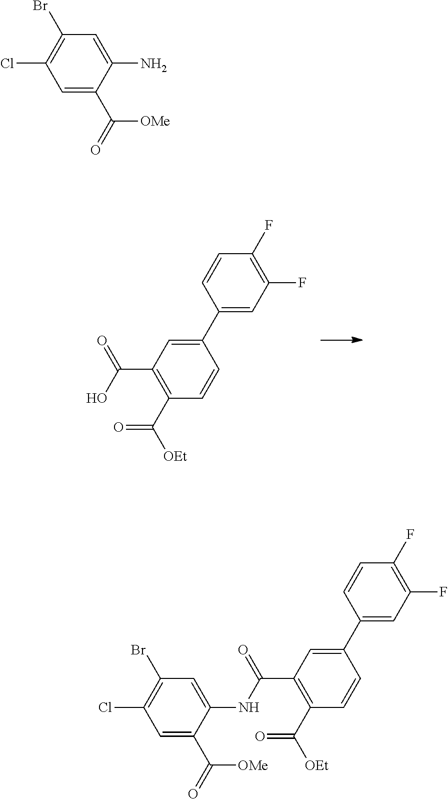 [SMILES: COC(=O)c1cc(Cl)c(Br)cc1N ; CCOC(=O)c1ccc(-c2ccc(F)c(F)c2)cc1C(=O)O ; CCOC(=O)c1ccc(-c2ccc(F)c(F)c2)cc1C(=O)Nc1cc(Br)c(Cl)cc1C(=O)OC]

Using the procedure described above for the synthesis of ethyl 3-((4-bromo-5-chloro-2-(methoxy-carbonyl)phenyl)carbamoyl)-3′,4′-difluoro-[1,1′-biphenyl]-4-carboxylate, methyl 2-amino-4-bromo-5-chlorobenzoate (0.154 g) was coupled with 4-(ethoxycarbonyl)-3′,4′-difluoro-[1,1′-biphenyl]-3-carboxylic acid (0.143 g) to afford ethyl 3-((5-bromo-4-chloro-2-(methoxycarbonyl)phenyl)carbamoyl)-3′,4′-difluoro-[1,1′-biphenyl]-4-carboxylate (0.183 g, 71% yield) after flash chromatography purification (SiO₂, hexane-ethyl acetate (0-80%).

### Step 20.2 Preparation of 3-((2-carboxy-4-chloro-5-(1H-1,2,3-triazol-5-yl)phenyl)carbamoyl)-3′,4′-difluoro-[1,1′-biphenyl]-4-carboxylic Acid

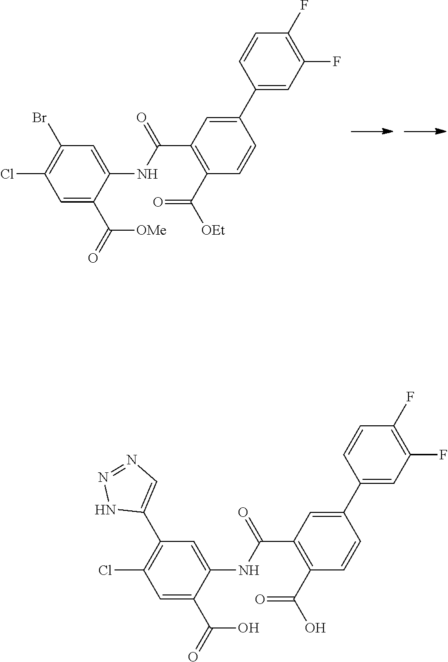 [SMILES: CCOC(=O)c1ccc(-c2ccc(F)c(F)c2)cc1C(=O)Nc1cc(Br)c(Cl)cc1C(=O)OC ; O=C(O)c1cc(Cl)c(-c2cnn[nH]2)cc1NC(=O)c1cc(-c2ccc(F)c(F)c2)ccc1C(=O)O]

Using General Procedures 3 and 4, ethyl 3-((5-bromo-4-chloro-2-(methoxycarbonyl)phenyl)carbamoyl)-3′,4′-difluoro-[1,1′-biphenyl]-4-carboxylate (0.183 g) was transformed into 3-((2-carboxy-4-chloro-5-(1H-1,2,3-triazol-5-yl)phenyl)carbamoyl)-3′,4′-difluoro-[1,1′-biphenyl]-4-carboxylic acid which was purified by preparative HPLC.

1H NMR (250 MHz, DMSO-d6) δ ppm 7.51-7.71 (m, 3H) 7.74-7.83 (m, 1H) 7.92-8.02 (m, 3H) 8.03-8.08 (m, 1H) 8.11 (s, 2H) 11.54 (d, J=13.40 Hz, 2H)

### Example 21: 3-(2-carboxy-5-(1H-1,2,3-triazol-5-yl)benzamido)-3′,4′-difluoro-[1,1′-biphenyl]-4-carboxylic acid (GO-0003580)

3-(2-carboxy-5-(1H-1,2,3-triazol-5-yl)benzamido)-3′,4′-difluoro-[1,1′-biphenyl]-4-carboxylic acid was prepared in several steps.

### Step 21.1 Preparation of dimethyl 4-bromophthalate

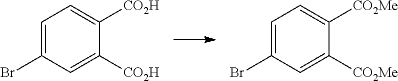 [SMILES: O=C(O)c1ccc(Br)cc1C(=O)O ; COC(=O)c1ccc(Br)cc1C(=O)OC]

4-bromophthalic acid (5 g, 20.4 mmol) was dissolved in methanol (100 mL) and treated with sulfuric acid (1 mL) and dimethyl sulfate (5 mL, 52.7 mmol). The mixture was heated overnight at 95° C. The methanol was removed and the residue was neutralized by the slow addition of sodium bicarbonate (50 mL, saturated aqueous). Sodium carbonate (4.5 g) was added and the mixture was extracted with ethyl acetate (3×), the combine solution was dried over sodium sulfate, filtered and concentrated. The residue was purified by flash chromatography (silica gel, ethyl acetate 0-50% in hexane) to afford pure dimethyl 4-bromophthalate (5.5 g, 99% yield).

### Step 21.2 Preparation of dimethyl 4-((trimethylsilyl)ethynyl)phthalate

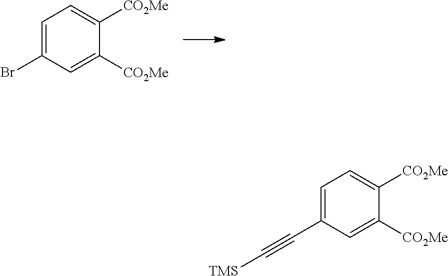 [SMILES: COC(=O)c1ccc(Br)cc1C(=O)OC ; COC(=O)c1ccc(C#C[Si](C)(C)C)cc1C(=O)OC]

Dimethyl 4-bromophthalate (5.56 g, 20.36 mmol) was dissolved in toluene (100 mL) and treated with TMS-acetylene (4.3 mL, 30.54 mmol), PdCl₂(PPh₃)₂ (0.715 g, 1.018 mmol), CuI (0.155 mg, 0.81 mmol), triethyl amine (9.4 mL, 67.2 mmol) and stirred at 85° C. for 2 h. The cooled reaction mixture was filtered through Celite, concentrated and the residue purified by flash chromatography (ethyl acetate 0-50% in hexane) to afford pure dimethyl 4-((trimethylsilyl)ethynyl)phthalate (5.85 g, 99% yield).

### Step 21.3 Preparation of dimethyl 4-ethynylphthalate

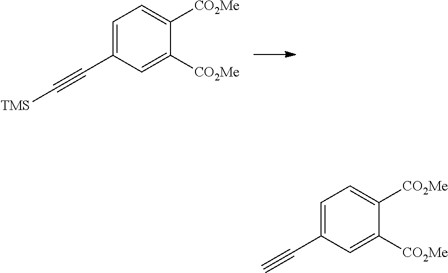 [SMILES: COC(=O)c1ccc(C#C[Si](C)(C)C)cc1C(=O)OC ; C#Cc1ccc(C(=O)OC)c(C(=O)OC)c1]

Dimethyl 4-((trimethylsilyl)ethynyl)phthalate (5.85 g, 19.97 mmol) was dissolved in a mixture of methanol (100 mL) and dichloromethane (10 mL) and treated with potassium carbonate (5.5 g, 39.8 mmol) at room temperature for 0.5 h. After dilution with dichloromethane and filtration, the solution is poured into water and extracted with dichloromethane (3×). The combined organic solution was washed with water and dried over sodium sulfate. The residue after evaporation was purified by flash chromatography (silica gel, ethyl acetate 0-30% in hexane) to afford pure dimethyl 4-ethynylphthalate (3.53 g, 80% yield).

### Step 21.4 Preparation of dimethyl 4-(1H-1,2,3-triazol-5-yl)phthalate

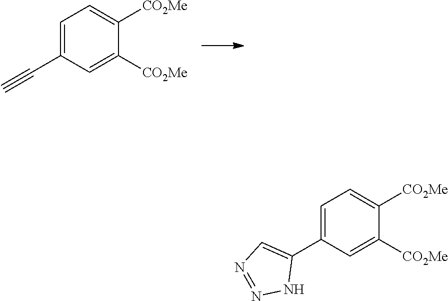 [SMILES: C#Cc1ccc(C(=O)OC)c(C(=O)OC)c1 ; COC(=O)c1ccc(-c2cnn[nH]2)cc1C(=O)OC]

Dimethyl 4-ethynylphthalate (0.595 g, 2.73 mmol) was dissolved in DMF (19 mL) and methanol (1.9 mL) and was treated with TMS-azide (1.07 mL, 8.18 mmol) and CuI (0.078 g, 0.41 mmol) in a microwave reactor at 100° C. for 6 h. The reaction was partitioned between water and ethyl acetate, and the aqueous layer was extracted with ethyl acetate (2×). The combined organic solution was washed with water and dried, filtered and evaporated to afford a residue which was purified by flash chromatography (silica gel, ethyl acetate 20-55% in hexane) to afford pure dimethyl 4-(1H-1,2,3-triazol-5-yl)phthalate (0.490 g, 69% yield).

### Step 21.5 Preparation of 3-(1,3-dioxo-5-(1H-1,2,3-triazol-5-yl)isoindolin-2-yl)-3′,4′-difluoro-[1,1′-biphenyl]-4-carboxylic acid

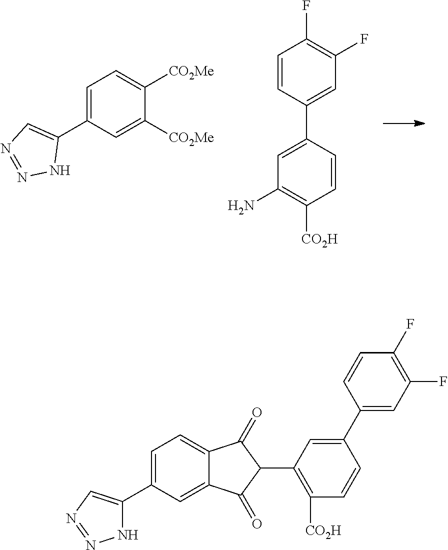 [SMILES: COC(=O)c1ccc(-c2cnn[nH]2)cc1C(=O)OC ; Nc1cc(-c2ccc(F)c(F)c2)ccc1C(=O)O ; O=C(O)c1ccc(-c2ccc(F)c(F)c2)cc1C1C(=O)c2ccc(-c3cnn[nH]3)cc2C1=O]

Dimethyl 4-(1H-1,2,3-triazol-5-yl)phthalate (0.300 g, 1.28 mmol) and 3-amino-3′,4′-difluoro-[1,1′-biphenyl]-4-carboxylic acid (0.321 g, 1.28 mmol) were dissolved in isobutyric acid (13 mL) and heated in a microwave reactor at 175° C. for 3 h. The cooled reaction mixture was evaporated to dryness. The residue was taken up in ethyl acetate and washed with water (2×) dried over sodium sulfate, filtered, concentrated and dried under high vacuum. To afford 3-(1,3-dioxo-5-(1H-1,2,3-triazol-5-yl)isoindolin-2-yl)-3′,4′-difluoro-[1,1′-biphenyl]-4-carboxylic acid (0.546 g, 95% yield).

### Step 21.6 Preparation of 3-(2-carboxy-5-(1H-1,2,3-triazol-5-yl)benzamido)-3′,4′-difluoro-[1,1′-biphenyl]-4-carboxylic acid and 3-[2-carboxy-4-(1H-1,2,3-triazol-5-yl)benzamido]-3′,4′-difluoro-[1,1′-biphenyl]-4-carboxylic Acid

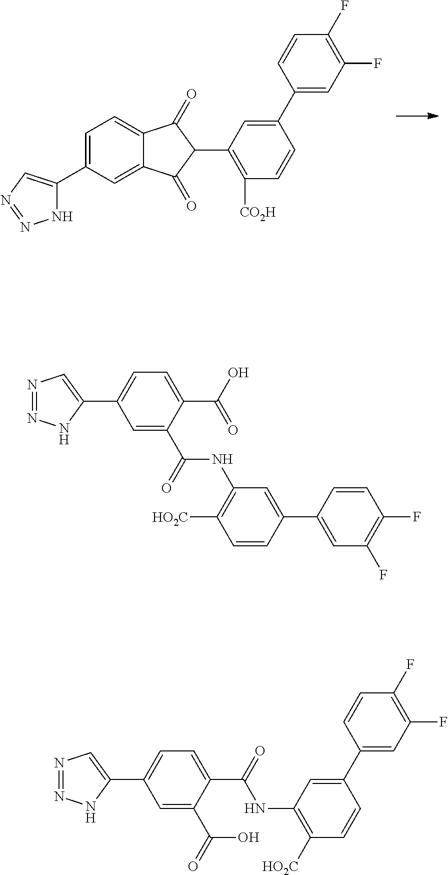 [SMILES: O=C(O)c1ccc(-c2ccc(F)c(F)c2)cc1C1C(=O)c2ccc(-c3cnn[nH]3)cc2C1=O ; O=C(O)c1ccc(-c2ccc(F)c(F)c2)cc1NC(=O)c1cc(-c2cnn[nH]2)ccc1C(=O)O ; O=C(O)c1ccc(-c2ccc(F)c(F)c2)cc1NC(=O)c1ccc(-c2cnn[nH]2)cc1C(=O)O]

3-(1,3-dioxo-5-(1H-1,2,3-triazol-5-yl)isoindolin-2-yl)-3′,4′-difluoro-[1,1′-biphenyl]-4-carboxylic acid (0.025 g, 0.056 mmol) was dissolved in methanol (0.6 mL) and treated with sodium hydroxide (0.14 mL, 2N, 0.28 mmol) and stirred for 40 min at room temperature. The mixture was acidified with HCl (0.2N) to pH-2 and then extracted with 2-methyltetrafuran (3×). The combined organic solution was washed with water and brine, dried over sodium sulfate, filtered and concentrated to dryness. The residue was separated by preparative HPLC to afford pure 3-(2-carboxy-5-(1H-1,2,3-triazol-5-yl)benzamido)-3′,4′-difluoro-[1,1′-biphenyl]-4-carboxylic acid and 3-(2-carboxy-4-(1H-1,2,3-triazol-5-yl)benzamido)-3′,4′-difluoro-[1,1′-biphenyl]-4-carboxylic acid. The target compound was selected as the isomer, which didn't match the NMR spectra of undesirable isomer.

### Example 22: 3-[2-carboxy-4-(1H-1,2,3-triazol-5-yl)benzamido]-3′,4′-difluoro-[1,1′-biphenyl]-4-carboxylic acid (GO-0003581)

3-[2-carboxy-4-(1H-1,2,3-triazol-5-yl)benzamido]-3′,4′-difluoro-[1,1′-biphenyl]-4-carboxylic acid was prepared at the Step 21.6 of Example 21. After preparative HPLC the target compound was selected as the isomer, which matched the NMR spectra.

1H NMR (500 MHz, DMSO-d6) δ ppm 7.52-7.63 (m, 3H) 7.74-7.84 (m, 2H) 8.00 (d, J=8.24 Hz, 1H) 8.08-8.15 (m, 2H) 8.18 (d, J=7.69 Hz, 1H) 8.34 (s, 1H) 8.92 (br. s., 1H) 11.64-11.72 (m, 1H)

### Example 23: 2-((4-carboxy-4′-fluoro-[1,1′-biphenyl]-3-yl)carbamoyl)terephthalic acid (GO-0003583)

2-((4-carboxy-4′-fluoro-[1,1′-biphenyl]-3-yl)carbamoyl)terephthalic acid was prepared at the Step 1.3 of Example 1. After preparative HPLC the target compound was selected as the isomer, which matched the NMR spectra.

1H NMR (250 MHz, DEUTERIUM OXIDE) δ ppm 7.39 (t, J=8.73 Hz, 2H) 7.53 (d, J=8.35 Hz, 1H) 7.71-7.86 (m, 3H) 8.10 (d, J=8.24 Hz, 1H) 8.23 (d, J=7.91 Hz, 1H) 8.40 (s, 1H) 8.87 (s, 1H)

### Example 26: 3-(5-chloro-2-(N,N-dimethylsulfamoyl)-4-(1H-1,2,3-triazol-5-yl)benzamido)-3′,4′-difluoro-[1,1′-biphenyl]-4-carboxylic acid (GO-0003605)

3-(5-chloro-2-(N,N-dimethylsulfamoyl)-4-(1H-1,2,3-triazol-5-yl)benzamido)-3′,4′-difluoro-[1,1′-biphenyl]-4-carboxylic acid was prepared in several steps.

### Step 26.1 Preparation of 5-bromo-4-chloro-2-methylbenzenesulfonyl chloride

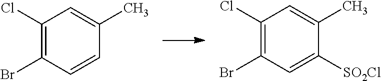

1-bromo-2-chloro-4-methylbenzene (1.0 g, 4.87 mmol) was added to chlorosulfonic acid (2 mL) with stirring at 0° C. The mixture was stirred for 30 min at this temperature then at room temperature for another 30 min. The reaction was then heated to 60° C. for 1 h and cooled and the mixture added dropwise into ice water. The precipitate thus formed was filtered and washed with water and dried under vacuum. The crude 5-bromo-4-chloro-2-methylbenzenesulfonyl chloride was used without further purification in the next step.

### Step 26.2 Preparation of 5-bromo-4-chloro-N,N,2-trimethylbenzenesulfonamide

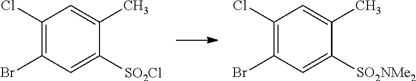

The crude 5-bromo-4-chloro-2-methylbenzenesulfonyl chloride (1.0 g, 3.29 mmol) was dissolved in THE (15 mL) and treated first with triethylamine (0.46 mL, 3.29 mmol) and then with dimethylamine (1.8 mL, 2M in THF, 3.6 mmol). The reaction mixture was stirred at room temperature for 3 h and then evaporated to dryness to afford crude 5-bromo-4-chloro-N,N,2-trimethylbenzenesulfonamide. No further purification was done on this material.

### Step 26.3 Preparation of 4-bromo-5-chloro-2-(N,N-dimethylsulfamoyl)benzoic acid

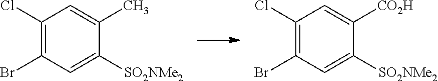

Crude 5-bromo-4-chloro-N,N,2-trimethylbenzenesulfonamide (0.85 g, 2.7 mmol) was dissolved in a mixture of water (10 mL) and t-BuOH (10 mL) and treated with potassium permanganate (2.14 g, 13.59 mmol) at 100° C. for 7 h. Most of the t-BuOH was removed from the cooled solution under reduced pressure and the remaining aqueous solution was filtered through Celite and the filter pad was rinsed with hot water. The filtered solution was acidified with 2N HCl to a pH of ˜2 and the mixture was extracted with ethyl acetate three times. The combined extracts were washed with water twice followed by brine, dried over sodium sulfate, filtered, concentrated and purified by flash chromatography (silica gel, dichloromethane-methanol (0 to 20%) to afford pure 4-bromo-5-chloro-2-(N,N-dimethylsulfamoyl)benzoic acid (0.375 g, 36% yield).

### Step 26.4 Preparation of methyl 3-(4-bromo-5-chloro-2-(N,N-dimethylsulfamoyl)benzamido)-3′,4′-difluoro-[1,1′-biphenyl]-4-carboxylate

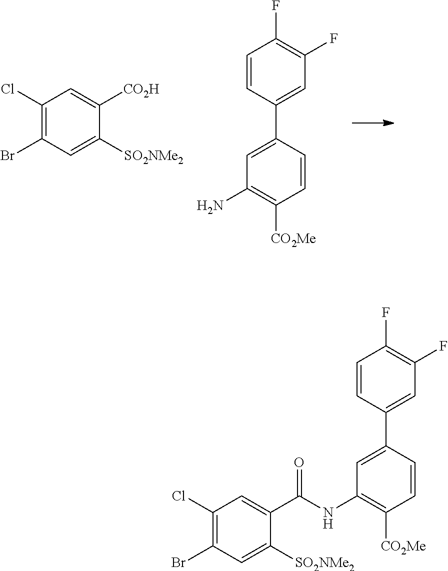

4-bromo-5-chloro-2-(N,N-dimethylsulfamoyl)benzoic acid(0.300 g, 0.88 mmol) and methyl 3-amino-3′,4′-difluoro-[1,1′-biphenyl]-4-carboxylate (0.431 g, were mixed in pyridine (26 mL) and cooled to 0° C. Phosphorus oxychloride (0.609 g, 3.9 mmol) was added dropwise and the ice bath was removed. The mixture was allowed to stir at room temperature for 0.5 h, after which time the reaction mixture was poured onto ice and extracted with ethyl acetate three times. The combined organic layers were washed several times with water, then brine to remove the pyridine and then dried and evaporated to dryness. The crude from this reaction was combined with that of two other test runs (total of 0.574 g of 4-bromo-5-chloro-2-(N,N-dimethylsulfamoyl)benzoic acid) and purified together by flash chromatography (silica gel, hexane-ethyl acetate (0 to 60%)) to afford pure methyl 3-(4-bromo-5-chloro-2-(N,N-dimethylsulfamoyl)benzamido)-3′,4′-difluoro-[1,1′-biphenyl]-4-carboxylate (0.236 g, 24% combined yield).

### Step 26.5 Preparation of methyl 3-(5-chloro-2-(N,N-dimethylsulfamoyl)-4-((trimethylsilyl)ethynyl)benzamido)-3′,4′-difluoro-[1,1′-biphenyl]-4-carboxylate

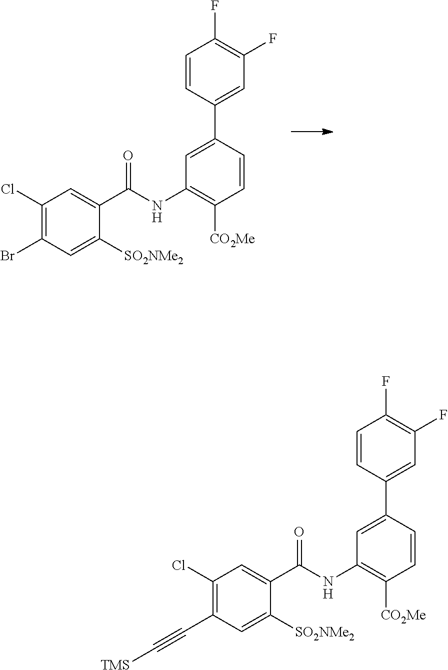

Methyl 3-(4-bromo-5-chloro-2-(N,N-dimethylsulfamoyl)benzamido)-3′,4′-difluoro-[1,1′-biphenyl]-4-carboxylate (0.215 g, 0.366 mmol) was dissolved in anhydrous DMF (6.5 mL). To this solution was added PdCl₂(PPh₃)₂ (0.051 g, 0.073 mmol), CuI (0.014 g, 0.073 mmol, trimethylsilyl acetylene (0.52 mL, 3.66 mmol) and triethylamine (0.51 mL, 3.66 mmol). The mixture was stirred at 50° C. for 1 h at which time the cooled solution was poured into water and extracted three times with ethyl acetate. The combined organic layers were washed with water, dried over sodium sulfate, filtered, concentrated and purified by flash chromatography (silica gel, hexane-ethyl acetate (0 to 70%)) to afford pure methyl 3-(5-chloro-2-(N,N-dimethylsulfamoyl)-4-((trimethylsilyl)ethynyl)benzamido)-3′,4′-difluoro-[1,1′-biphenyl]-4-carboxylate (0.146 g, 58% yield).

### Step 26.6 Preparation of methyl 3-(5-chloro-2-(N,N-dimethylsulfamoyl)-4-ethynylbenzamido)-3′,4′-difluoro-[1,1′-biphenyl]-4-carboxylate

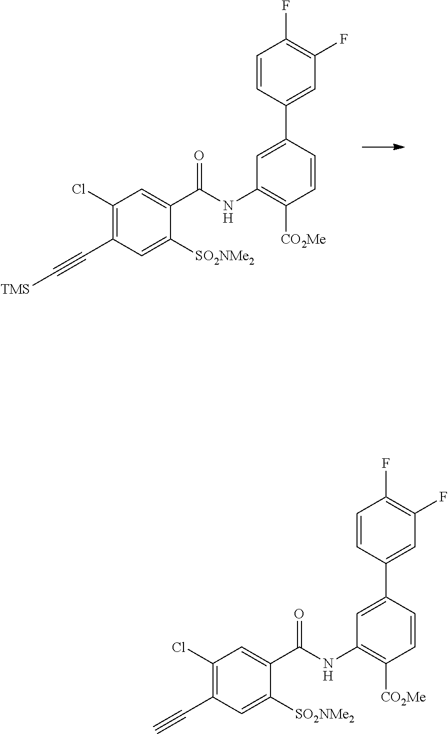

Methyl 3-(5-chloro-2-(N,N-dimethylsulfamoyl)-4-((trimethylsilyl)ethynyl)benzamido)-3′,4′-difluoro-[1,1′-biphenyl]-4-carboxylate (0.146 g, 0.24 mmol) was dissolved in a 1:1 mixture of methanol:dichloromethane (6 mL) and treated with potassium carbonate (0.069 g, 0.5 mmol) for 25 min at room temperature. The reaction was partitioned between ethyl acetate and 0.2N HCl. The aqueous layer was extracted with ethyl acetate (3×) and the combined organic layers were washed with water (2×) and brine, and then dried over sodium sulfate. Filtration, concentration to dryness and drying under vacuum afforded a crude product which was used in the next step

### Step 26.7 Preparation of methyl 3-(5-chloro-2-(N,N-dimethylsulfamoyl)-4-(1H-1,2,3-triazol-5-yl)benzamido)-3′,4′-difluoro-[1,1′-biphenyl]-4-carboxylate

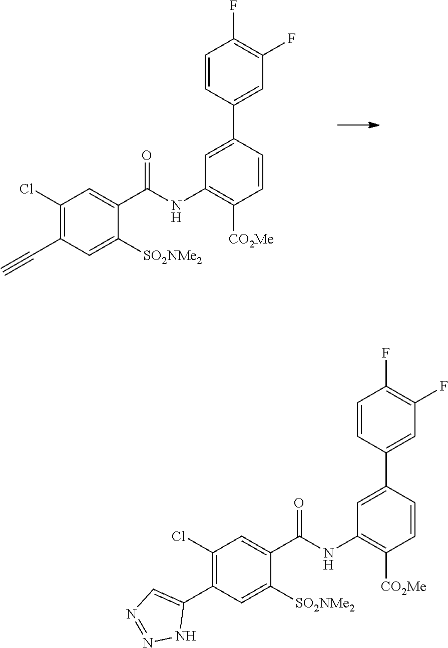

Methyl 3-(5-chloro-2-(N,N-dimethylsulfamoyl)-4-ethynylbenzamido)-3′,4′-difluoro-[1,1′-biphenyl]-4-carboxylate (0.123 g, 0.23 mmol) was dissolved in DMF (3.7 mL) and methanol (0.37 mL). To this solution was added TMS azide (303 μL, 2.3 mmol) and CuI (0.009 g, 0.046 mmol) and the mixture was heated at 100° C. in a microwave reactor for 10 min. The cooled reaction mixture was poured into water and extracted with 2-methyl THE (3×). The combined organic layers were dried over sodium sulfate, filtered, concentrated, dried under vacuum and used in the next step. Crude yield was 0.216 g.

### Step 26.7 Preparation of 3-(5-chloro-2-(N,N-dimethylsulfamoyl)-4-(1H-1,2,3-triazol-5-yl)benzamido)-3′,4′-difluoro-[1,1′-biphenyl]-4-carboxylic acid

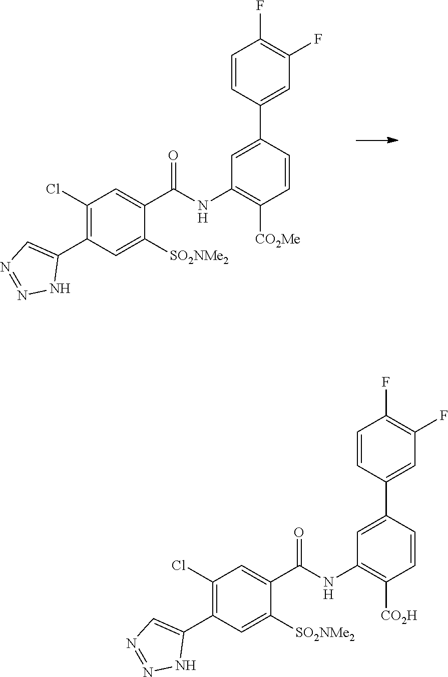

The crude methyl 3-(5-chloro-2-(N,N-dimethylsulfamoyl)-4-(1H-1,2,3-triazol-5-yl)benzamido)-3′,4′-difluoro-[1,1′-biphenyl]-4-carboxylate (0.216 g, 0.375 mmol) from the previous step was dissolved in a 1:1 mixture of methanol and THF (10 mL) and treated with 2N NaOH(0.94 mL, 5 equiv). After stirring at room temperature for 2 h, the solution was acidified with 0.2N HCl to pH-2. The organic solvent was evaporated and the mixture was extracted with ethyl acetate (3×). The combined organic layers were washed with water (2×), then brine, dried over sodium sulfate, filtered, concentrated and dried under vacuum to afford a solid which was subjected to final purification by HPLC.

1H NMR (250 MHz, DMSO-d6) δ ppm 2.77 (s, 6H) 7.51-7.68 (m, 3H) 7.74-7.87 (m, 1H) 8.09 (t, J=4.18 Hz, 2H) 8.36-8.55 (m, 1H) 8.72 (d, J=1.10 Hz, 1H) 11.44 (s, 1H)

### Example 27: 4-((3′,4′-difluoro-4-hydroxy-[1,1′-biphenyl]-3-yl)carbamoyl)-6-hydroxyisophthalic acid (GO-0003609)

4-((3′,4′-difluoro-4-hydroxy-[1,1′-biphenyl]-3-yl)carbamoyl)-6-hydroxyisophthalic acid was prepared in several steps.

### Step 27.1 Preparation of 3-amino-3′,4′-difluoro-[1,1′-biphenyl]-4-ol

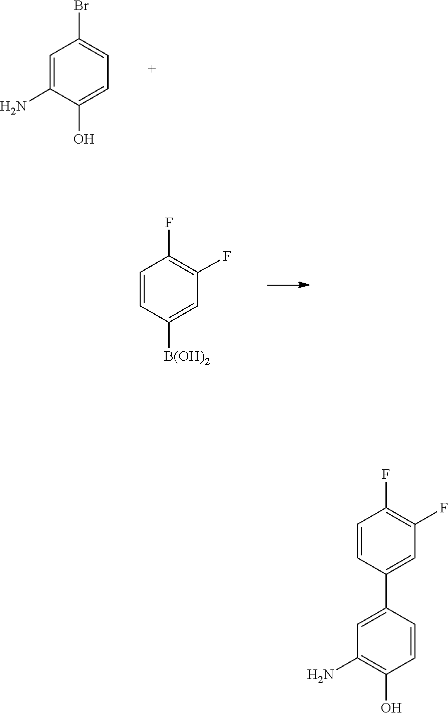 [SMILES: Nc1cc(Br)ccc1O ; OB(O)c1ccc(F)c(F)c1 ; Nc1cc(-c2ccc(F)c(F)c2)ccc1O]

A mixture of 2-amino-4-bromophenol (1 g, 5.318 mmol), (3,4-difluorophenyl)boronic acid (0.840 g, 5.318 mmol), tetrakis-triphenylphosphine palladium (0) (0.308 g, 0.266 mmol) and potassium carbonate (1.47 g, 10.6 mmol) was heated in dioxane (17 mL) and water (4.5 mL) at 120° C. in a microwave reactor for 3 h. The cooled mixture was filtered and the filtrate was extracted three times with a mixture of 2-methyltetrahydrofuran and ethyl acetate (1:1). The combined organic solution was dried over sodium sulfate, filtered, evaporated to dryness and the residue was purified by flash chromatography (silica gel, ethyl acetate 0-100% in hexane) to afford pure 3-amino-3′,4′-difluoro-[1,1′-biphenyl]-4-ol (0.318 g, 27% yield).

### Step 27.2 Preparation of 2-(3′,4′-difluoro-4-hydroxy-[1,1′-biphenyl]-3-yl)-6-hydroxy-1,3-dioxoisoindoline-5-carboxylic acid

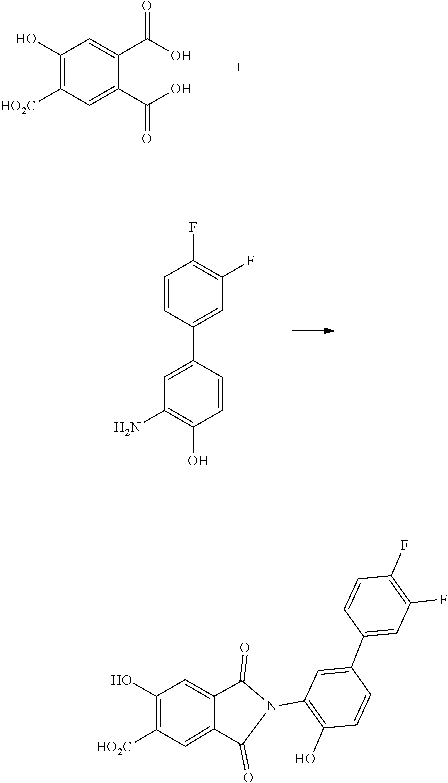 [SMILES: O=C(O)c1cc(C(=O)O)c(C(=O)O)cc1O ; Nc1cc(-c2ccc(F)c(F)c2)ccc1O ; O=C(O)c1cc2c(cc1O)C(=O)N(c1cc(-c3ccc(F)c(F)c3)ccc1O)C2=O]

A mixture of 5-hydroxybenzene-1,2,4-tricarboxylic acid (0.204 g, 0.904 mmol) and 3-amino-3′,4′-difluoro-[1,1′-biphenyl]-4-ol (0.200 g, 0.904 mmol) was dissolved in isobutyric acid (9 mL) and heated at 175° C. in a microwave reactor for 3 h. The solvent was removed from the cooled reaction mixture under reduced pressure and the residue was purified by flash chromatography (silica gel, methanol 0-20% in dichloromethane) to afford pure 2-(3′,4′-difluoro-4-hydroxy-[1,1′-biphenyl]-3-yl)-6-hydroxy-1,3-dioxoisoindoline-5-carboxylic acid (0.291 g, 79% yield).

### Step 27.3 Preparation of 6-acetoxy-2-(4-acetoxy-3′,4′-difluoro-[1,1′-biphenyl]-3-yl)-1,3-dioxoisoindoline-5-carboxylic Acid

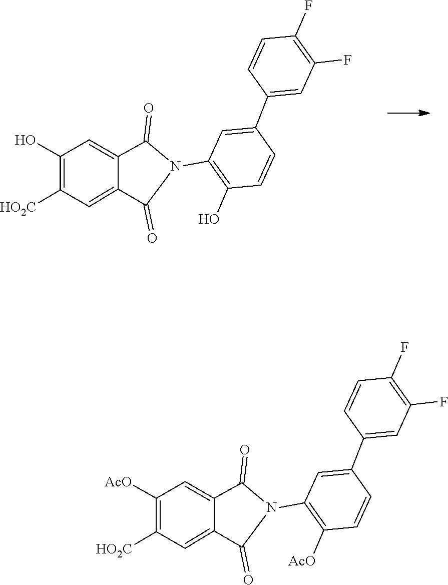 [SMILES: O=C(O)c1cc2c(cc1O)C(=O)N(c1cc(-c3ccc(F)c(F)c3)ccc1O)C2=O ; CC(=O)Oc1cc2c(cc1C(=O)O)C(=O)N(c1cc(-c3ccc(F)c(F)c3)ccc1OC(C)=O)C2=O]

2-(3′,4′-difluoro-4-hydroxy-[1,1′-biphenyl]-3-yl)-6-hydroxy-1,3-dioxoisoindoline-5-carboxylic acid (0.231 g, 0.562 mmol) was dissolved in acetic anhydride (10 mL) and then treated with sulfuric acid (5 drops). The mixture was stirred at room temperature overnight at which time it was poured into water and extracted with ethyl acetate (3×). The combined organic solution was washed with water (2×) and brine, then dried over sodium sulfate, filtered and evaporated to dryness and dried under high vacuum. The crude material, 6-acetoxy-2-(4-acetoxy-3′,4′-difluoro-[1,1′-biphenyl]-3-yl)-1,3-dioxoisoindoline-5-carboxylic acid, was used in the following step without further purification.

### Step 27.4 Preparation of 4-((3′,4′-difluoro-4-hydroxy-[1,1′-biphenyl]-3-yl)carbamoyl)-6-hydroxyisophthalic acid and 2-((3′,4′-difluoro-4-hydroxy-[1,1′-biphenyl]-3-yl)carbamoyl)-5-hydroxyterephthalic acid

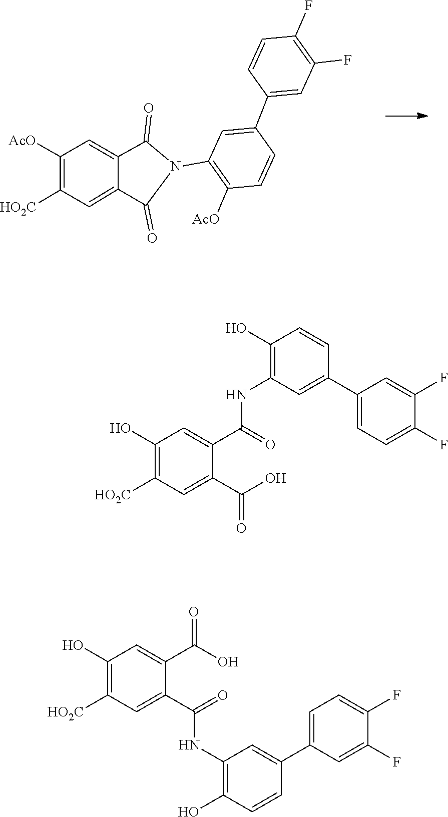 [SMILES: CC(=O)Oc1cc2c(cc1C(=O)O)C(=O)N(c1cc(-c3ccc(F)c(F)c3)ccc1OC(C)=O)C2=O ; O=C(O)c1cc(C(=O)O)c(C(=O)Nc2cc(-c3ccc(F)c(F)c3)ccc2O)cc1O ; O=C(O)c1cc(C(=O)Nc2cc(-c3ccc(F)c(F)c3)ccc2O)c(C(=O)O)cc1O]

The crude 6-acetoxy-2-(4-acetoxy-3′,4′-difluoro-[1,1′-biphenyl]-3-yl)-1,3-dioxoisoindoline-5-carboxylic acid from the previous step was dissolved in a mixture of THF and methanol (7 mL+7 mL) and treated with sodium hydroxide (2.8 mL, 2 N). The mixture was stirred at 55° C. for 0.5 h. The cooled solution was acidified with HCl (5.6 mL) and the solvent was evaporated. The residue was partitioned between HCl (0.2 N) and ethyl acetate. The aqueous layer was extracted twice more with ethyl acetate. The combined organic solution was washed with water (2×) and brine, then dried over sodium sulfate, filtered and evaporated to dryness and dried under high vacuum. Purification by preparative HPLC afforded 4-((3′,4′-difluoro-4-hydroxy-[1,1′-biphenyl]-3-yl)carbamoyl)-6-hydroxyisophthalic acid and 2-((3′,4′-difluoro-4-hydroxy-[1,1′-biphenyl]-3-yl)carbamoyl)-5-hydroxyterephthalic acid. The target compound was selected as the isomer, which matched the NMR spectra.

NMR H1:

### Example 28: 2-({4-carboxy-3′,4′-difluoro-[1,1′-biphenyl]-3-yl}carbamoyl)-5-hydroxybenzene-1,4-dicarboxylic acid (GO-0003613)

2-({4-carboxy-3′,4′-difluoro-[1,1′-biphenyl]-3-yl}carbamoyl)-5-hydroxybenzene-1,4-dicarboxylic acid was prepared at the Step 16.1 of Example 16. After preparative HPLC the target compound was selected as the isomer, which matched the NMR spectra.

1H NMR (250 MHz, DMSO-d6) δ ppm 7.26 (d, J=1.32 Hz, 1H) 7.48-7.69 (m, 3H) 7.74-7.86 (m, 1H) 8.06-8.14 (m, 1H) 8.20 (s, 1H) 8.86 (s, 1H)

### Example 29: 2-{[3-cyano-4-(4-methoxyphenyl)-5-methylthiophen-2-yl]carbamoyl}benzene-1,4-dicarboxylic acid (GO-0003614)

2-{[3-cyano-4-(4-methoxyphenyl)-5-methylthiophen-2-yl]carbamoyl}benzene-1,4-dicarboxylic acid was prepared in several steps.

### Step 29.1 Preparation of 2-(3-cyano-4-(4-methoxyphenyl)-5-methylthiophen-2-yl)-1,3-dioxoisoindoline-5-carboxylic Acid

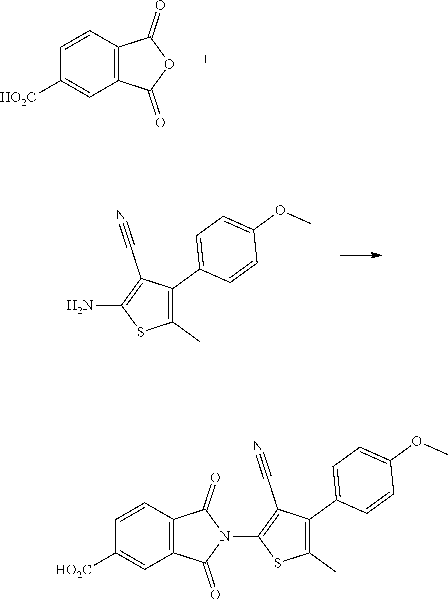 [SMILES: O=C(O)c1ccc2c(c1)C(=O)OC2=O ; COc1ccc(-c2c(C)sc(N)c2C#N)cc1 ; COc1ccc(-c2c(C)sc(N3C(=O)c4ccc(C(=O)O)cc4C3=O)c2C#N)cc1]

A solution of 1,3-dioxo-1,3-dihydroisobenzofuran-5-carboxylic acid (1.58 g, 8.18 mmol) and 2-amino-4-(4-methoxyphenyl)-5-methylthiophene-3-carbonitrile (2.0 g, 8.18 mmol) in acetic acid (80 mL) was heated at 120° C. for 23 h. Upon cooling, a precipitate formed which was filtered, rinsed with water and dried under high vacuum to afford pure 2-(3-cyano-4-(4-methoxyphenyl)-5-methylthiophen-2-yl)-1,3-dioxoisoindoline-5-carboxylic acid (2.38 g, 70% yield). The filtered solution was evaporated to dryness, dissolved in ethyl acetate, washed with water, dried over sodium sulfate, filtered and concentrated to afford a solid. The solid was purified by flash chromatography to afford an additional 0.185 g pure product.

### Step 29.2 Preparation of 2-((4-carboxy-3′,4′-difluoro-[1,1′-biphenyl]-3-yl)carbamoyl)-5-chloroterephthalic acid and 4-((4-carboxy-3′,4′-difluoro-[1,1′-biphenyl]-3-yl)carbamoyl)-6-chloroisophthalic acid

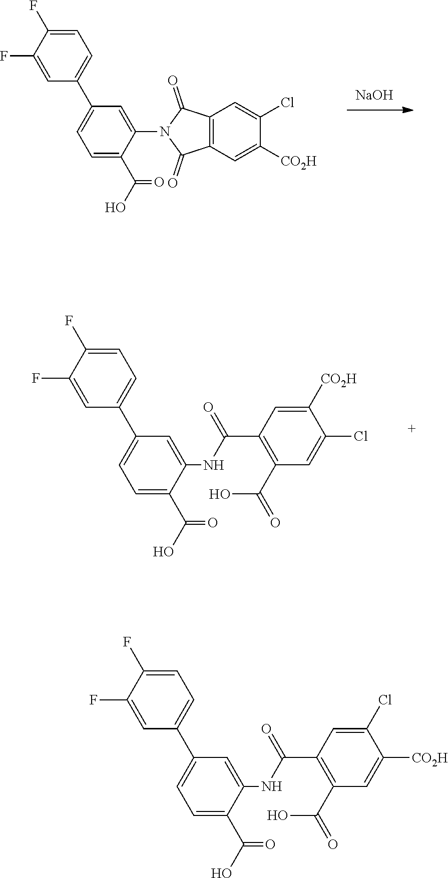 [SMILES: O=C(O)c1cc2c(cc1Cl)C(=O)N(c1cc(-c3ccc(F)c(F)c3)ccc1C(=O)O)C2=O ; "NaOH" ; O=C(O)c1cc(C(=O)Nc2cc(-c3ccc(F)c(F)c3)ccc2C(=O)O)c(C(=O)O)cc1Cl ; O=C(O)c1cc(C(=O)O)c(C(=O)Nc2cc(-c3ccc(F)c(F)c3)ccc2C(=O)O)cc1Cl]

2-(4-carboxy-3′,4′-difluoro-[1,1′-biphenyl]-3-yl)-6-chloro-1,3-dioxoisoindoline-5-carboxylic acid. (0.035 g, 0.0764 mmol) was dissolved in THF (1 mL) and methanol (1 mL) and treated with sodium hydroxide (0.38 mL, 2M aqueous, 10 equiv). After stirring at room temperature for 1 h the reaction mixture was poured into HCl (0.2 M) and extracted with ethyl acetate (4×). The combined organics were washed with water, brine, dried over sodium sulfate. Concentration to dryness afforded a residue which was purified by preparative HPLC to afford pure 2-((4-carboxy-3′,4′-difluoro-[1,1′-biphenyl]-3-yl)carbamoyl)-5-chloroterephthalic acid and 4-((4-carboxy-3′,4′-difluoro-[1,1′-biphenyl]-3-yl)carbamoyl)-6-chloroisophthalic acid. After preparative HPLC the target compound was selected as the isomer, which matched the NMR spectra.

1H NMR (250 MHz, DMSO-d6) δ ppm 2.30 (s, 3H) 3.81 (s, 3H) 7.06 (m, J=8.57 Hz, 2H) 7.33 (m, J=8.35 Hz, 2H) 7.99-8.19 (m, 3H)

### Example 30: 4-{[3-cyano-4-(4-methoxyphenyl)-5-methylthiophen-2-yl]carbamoyl}benzene-1,3-dicarboxylic acid (GO-0003615)

4-{[3-cyano-4-(4-methoxyphenyl)-5-methylthiophen-2-yl]carbamoyl}benzene-1,3-dicarboxylic acid was prepared at the Step 29.2 of Example 29. After preparative HPLC the target compound was selected as the isomer, which matched the NMR spectra.

1H NMR (250 MHz, DMSO-d6) δ ppm 2.30 (s, 3H) 3.82 (s, 3H) 7.06 (m, J=7.91 Hz, 2H) 7.33 (m, J=7.47 Hz, 2H) 7.70 (d, J=7.91 Hz, 1H) 8.20 (d, J=7.91 Hz, 1H) 8.48 (s, 1H)

### Example 32: 3-(4-carboxy-2-(dimethylcarbamoyl)-5-hydroxybenzamido)-2′,4′-dichloro-[1,1′-biphenyl]-4-carboxylic acid (GO-0003617)

3-(4-carboxy-2-(dimethylcarbamoyl)-5-hydroxybenzamido)-2′,4′-dichloro-[1,1′-biphenyl]-4-carboxylic acid was prepared in several steps.

### Step 32.1 Preparation of 2-(2′,4′-dichloro-4-(methoxycarbonyl)-[1,1′-biphenyl]-3-yl)-6-hydroxy-1,3-dioxoisoindoline-5-carboxylic Acid

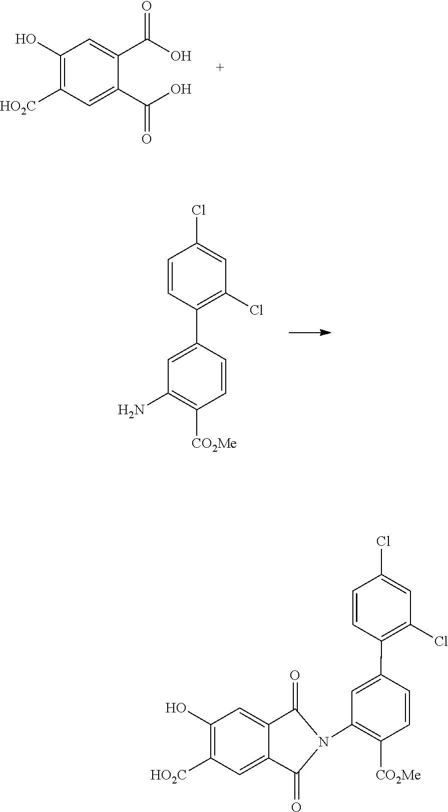 [SMILES: O=C(O)c1cc(C(=O)O)c(C(=O)O)cc1O ; COC(=O)c1ccc(-c2ccc(Cl)cc2Cl)cc1N ; COC(=O)c1ccc(-c2ccc(Cl)cc2Cl)cc1N1C(=O)c2cc(O)c(C(=O)O)cc2C1=O]

5-hydroxybenzene-1,2,4-tricarboxylic acid (0.34 g, 1.5 mmol) and methyl 3-amino-2′,4′-dichloro-[1,1′-biphenyl]-4-carboxylate (0.445 g, 1.5 mmol) were dissolved in isobutyric acid (15 mL) and heated in a microwave reactor, first at 140° C. for 1 h followed by 175° C. for 2 h. The cooled reaction mixture was evaporated to dryness and the residue purified by flash chromatography (silica gel, dichloromethane-methanol (0 to 20%)) to afford pure 2-(2′,4′-dichloro-4-(methoxycarbonyl)-[1,1′-biphenyl]-3-yl)-6-hydroxy-1,3-dioxoisoindoline-5-carboxylic acid (0.50 g, quantitative).

### Step 32.2 Preparation of 4-((2′,4′-dichloro-4-(methoxycarbonyl)-[1,1′-biphenyl]-3-yl)carbamoyl)-5-(dimethyl-carbamoyl)-2-hydroxybenzoic acid and 5-((2′,4′-dichloro-4-(methoxycarbonyl)-[1,1′-biphenyl]—3-yl)carbamoyl)-4-(dimethylcarbamoyl)-2-hydroxybenzoic acid

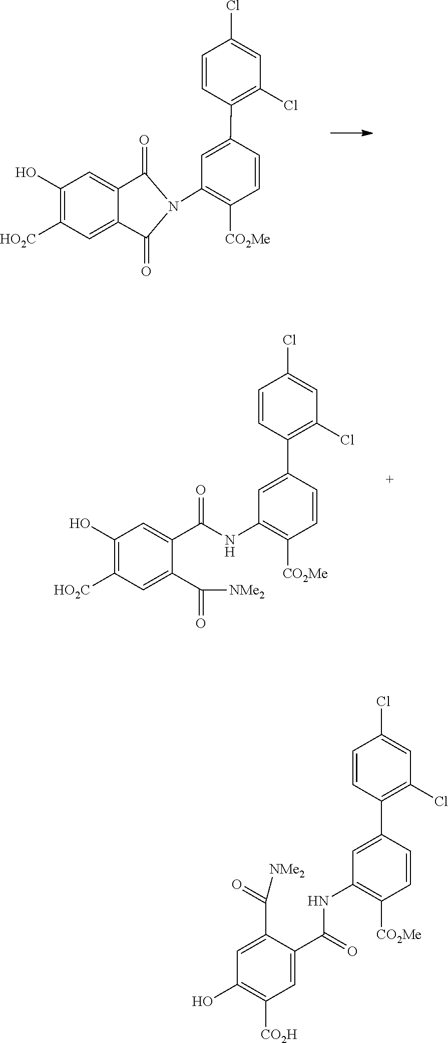 [SMILES: COC(=O)c1ccc(-c2ccc(Cl)cc2Cl)cc1N1C(=O)c2cc(O)c(C(=O)O)cc2C1=O ; COC(=O)c1ccc(-c2ccc(Cl)cc2Cl)cc1NC(=O)c1cc(O)c(C(=O)O)cc1C(=O)N(C)C ; COC(=O)c1ccc(-c2ccc(Cl)cc2Cl)cc1NC(=O)c1cc(C(=O)O)c(O)cc1C(=O)N(C)C]

2-(2′,4′-dichloro-4-(methoxycarbonyl)-[1,1′-biphenyl]-3-yl)-6-hydroxy-1,3-dioxoisoindoline-5-carboxylic acid (0.50 g, 1.0 mmol), was stirred in THF (4 mL) with dimethylamine (6 mL, 2M in THF) at room temperature for 1 h. The solvent was evaporated and the residue dried under high vacuum to afford a mixture of the two possible products of ring opening, 4-((2′,4′-dichloro-4-(methoxycarbonyl)-[1,1′-biphenyl]-3-yl)carbamoyl)-5-(dimethylcarbamoyl)-2-hydroxybenzoic acid and 5-((2′,4′-dichloro-4-(methoxycarbonyl)-[1,1′-biphenyl]-3-yl)carbamoyl)-4-(dimethylcarbamoyl)-2-hydroxybenzoic acid (0.54 g, 99% combined yield).

### Step 32.3 Preparation of 3-(4-carboxy-2-(dimethylcarbamoyl)-5-hydroxybenzamido)-2′,4′-dichloro-[1,1′-biphenyl]-4-carboxylic Acid

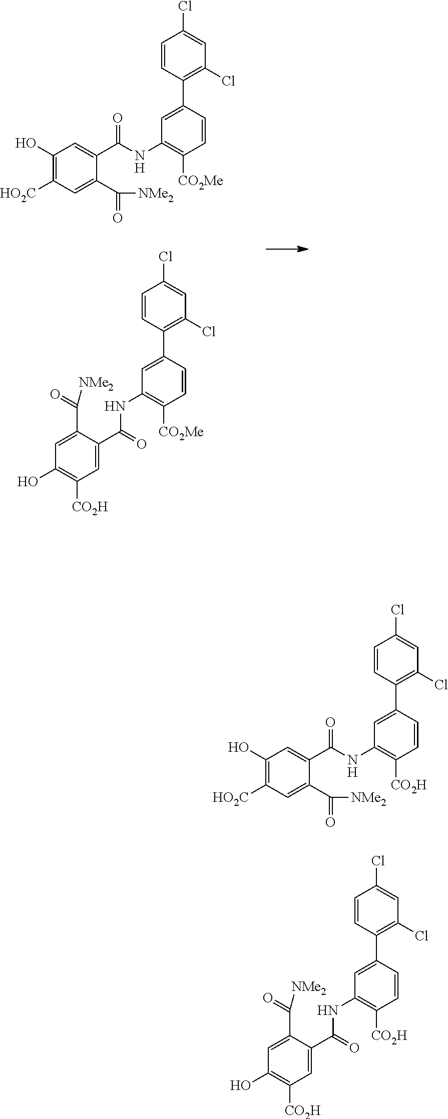 [SMILES: COC(=O)c1ccc(-c2ccc(Cl)cc2Cl)cc1NC(=O)c1cc(O)c(C(=O)O)cc1C(=O)N(C)C ; COC(=O)c1ccc(-c2ccc(Cl)cc2Cl)cc1NC(=O)c1cc(C(=O)O)c(O)cc1C(=O)N(C)C ; CN(C)C(=O)c1cc(C(=O)O)c(O)cc1C(=O)Nc1cc(-c2ccc(Cl)cc2Cl)ccc1C(=O)O ; CN(C)C(=O)c1cc(O)c(C(=O)O)cc1C(=O)Nc1cc(-c2ccc(Cl)cc2Cl)ccc1C(=O)O]

A portion of the product mixture from the previous step (0.20 g, 0.38 mmol) was dissolved in THF (6 mL) and methanol (6 mL) and treated with 2N NaOH (1 mL, 2 mmol) for 1.5 h at room temperature. The reaction mixture was partitioned between 0.2N HCl and ethyl acetate. The aqueous layer was further extracted with ethyl acetate (3×) and the combined organic layers were washed with water (2×), brine, dried over sodium sulfate, filtered and concentrated, and dried under vacuum. The resulting solid was submitted to HPLC purification to afford pure 3-(4-carboxy-2-(dimethylcarbamoyl)-5-hydroxybenzamido)-2′,4′-dichloro-[1,1′-biphenyl]-4-carboxylic acid (NSQP00676) and 3-(5-carboxy-2-(dimethylcarbamoyl)-4-hydroxybenzamido)-2′,4′-dichloro-[1,1′-biphenyl]-4-carboxylic acid. After preparative HPLC the target compound was selected as the isomer, which matched the NMR spectra.

1H NMR (250 MHz, DMSO-d6) δ ppm 2.74-2.81 (m, 3H) 2.93 (s, 3H) 6.84-6.96 (m, 1H) 7.26 (dd, J=8.24, 1.65 Hz, 1H) 7.47-7.63 (m, 2H) 7.81 (d, J=1.98 Hz, 1H) 8.13 (d, J=8.35 Hz, 1H) 8.37-8.45 (m, 1H) 8.61 (d, J=1.54 Hz, 1H) 12.11 (s, 1H)

### Example 33 4-((3-cyano-4-(4-methoxyphenyl)-5-methylthiophen-2-yl)carbamoyl)-6-hydroxyisophthalic acid (GO-0003620)

4-((3-cyano-4-(4-methoxyphenyl)-5-methylthiophen-2-yl)carbamoyl)-6-hydroxyisophthalic acid was prepared in several steps.

### Step 33.1 Preparation of 2-(3-cyano-4-(4-methoxyphenyl)-5-methylthiophen-2-yl)-6-hydroxy-1,3-dioxoisoindoline-5-carboxylic Acid

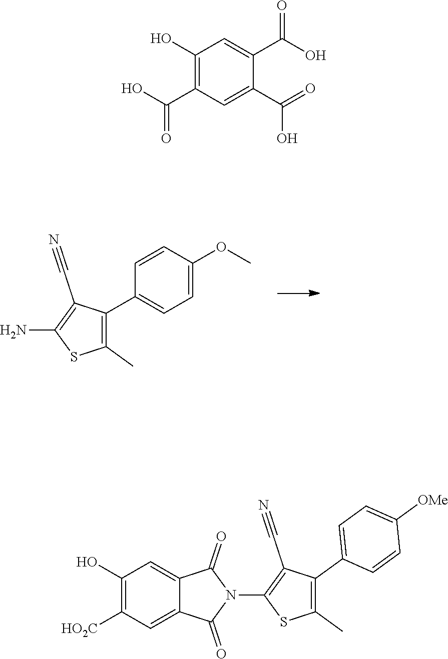 [SMILES: O=C(O)c1cc(C(=O)O)c(C(=O)O)cc1O ; COc1ccc(-c2c(C)sc(N)c2C#N)cc1 ; COc1ccc(-c2c(C)sc(N3C(=O)c4cc(O)c(C(=O)O)cc4C3=O)c2C#N)cc1]

Using General Procedure #5, the reaction of 5-hydroxybenzene-1,2,4-tricarboxylic acid (0.100 g) with 2-amino-4-(4-methoxyphenyl)-5-methylthiophene-3-carbonitrile (0.100 g) was carried out to afford 2-(3-cyano-4-(4-methoxyphenyl)-5-methylthiophen-2-yl)-6-hydroxy-1,3-dioxoisoindoline-5-carboxylic acid (0.180 g) after flash chromatography purification (SiO₂, dichloromethane-methanol, 0-15%).

### Step 33.2 Preparation of 4-((3-cyano-4-(4-methoxyphenyl)-5-methylthiophen-2-yl)carbamoyl)-6-hydroxyisophthalic acid and 2-((3-cyano-4-(4-methoxyphenyl)-5-methylthiophen-2-yl)carbamoyl)-5-hydroxyterephthalic acid

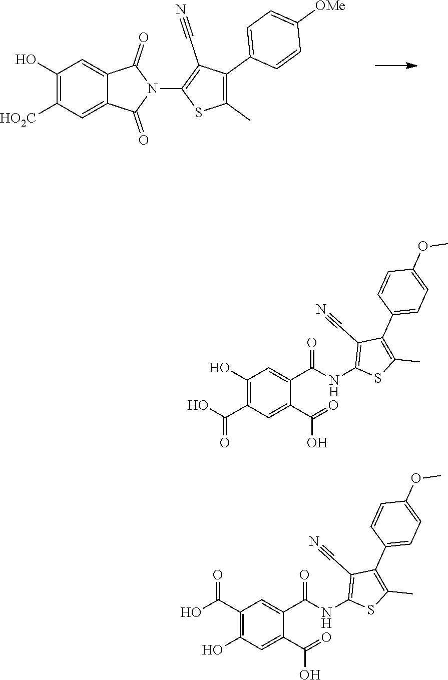 [SMILES: COc1ccc(-c2c(C)sc(N3C(=O)c4cc(O)c(C(=O)O)cc4C3=O)c2C#N)cc1 ; COc1ccc(-c2c(C)sc(NC(=O)c3cc(O)c(C(=O)O)cc3C(=O)O)c2C#N)cc1 ; COc1ccc(-c2c(C)sc(NC(=O)c3cc(C(=O)O)c(O)cc3C(=O)O)c2C#N)cc1]

The hydrolytic ring opening of 2-(3-cyano-4-(4-methoxyphenyl)-5-methylthiophen-2-yl)-6-hydroxy-1,3-dioxoisoindoline-5-carboxylic acid (0.140 g) with sodium hydroxide (1.6 mL, 2M aqueous) in methanol-THF (8 mL-8 mL) at 50° C. for 1.5 h afforded a mixture of 4-((3-cyano-4-(4-methoxyphenyl)-5-methylthiophen-2-yl)carbamoyl)-6-hydroxyisophthalic acid and 2-((3-cyano-4-(4-methoxyphenyl)-5-methylthiophen-2-yl)carbamoyl)-5-hydroxyterephthalic acid which were purified and separated by preparative HPLC. After preparative HPLC the target compound was selected as the isomer, which matched the NMR spectra.

1H NMR (250 MHz, DMSO-d6) δ ppm 2.29 (s, 3H) 3.82 (s, 3H) 7.06 (d, J=8.79 Hz, 2H) 7.21-7.40 (m, 3H) 7.88-8.00 (m, 1H) 12.07-12.19 (m, 1H)

### Example 34: 3-(2-carboxy-5-((dimethyl(oxo)-λ⁶-sulfanylidene)carbamoyl)benzamido)-2′,4′-dichloro-[1,1′-biphenyl]-4-carboxylic acid (GO-0003624)

3-(2-carboxy-5-((dimethyl(oxo)-λ⁶-sulfanylidene)carbamoyl)benzamido)-2′,4′-dichloro-[1,1′-biphenyl]-4-carboxylic acid was prepared in several steps.

### Step 34.1 Preparation of 2-(2′,4′-dichloro-4-(methoxycarbonyl)-[1,1′-biphenyl]-3-yl)-1,3-dioxoisoindoline-5-carboxylic Acid

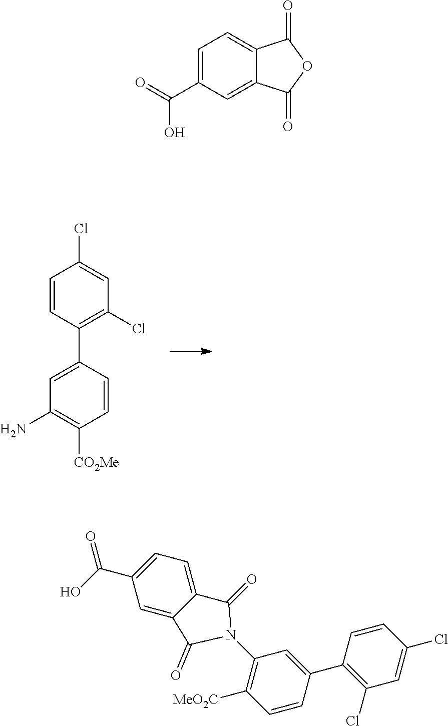 [SMILES: O=C(O)c1ccc2c(c1)C(=O)OC2=O ; COC(=O)c1ccc(-c2ccc(Cl)cc2Cl)cc1N ; COC(=O)c1ccc(-c2ccc(Cl)cc2Cl)cc1N1C(=O)c2ccc(C(=O)O)cc2C1=O]

1,3-dioxo-1,3-dihydroisobenzofuran-5-carboxylic acid (0.150 g, 0.78 mmol) and methyl 3-amino-2′,4′-dichloro-[1,1′-biphenyl]-4-carboxylate (0.220 g, 0.78 mmol) were dissolved in isobutyric acid (10 mL) and heated at 175° C. in a microwave reactor for 3 h. The cooled solution was evaporated to dryness and the product was purified by flash chromatography (silica gel, dichloromethane-methanol (0 to 20%)) to afford pure 2-(2′,4′-dichloro-4-(methoxycarbonyl)-[1,1′-biphenyl]-3-yl)-1,3-dioxoisoindoline-5-carboxylic acid (0.303 g, 82% yield).

### Step 34.2 Preparation of methyl 2′,4′-dichloro-3-(5-((dimethyl(oxo)-λ⁶-sulfanylidene)carbamoyl)-1,3-dioxoisoindolin-2-yl)-[1,1′-biphenyl]-4-carboxylate

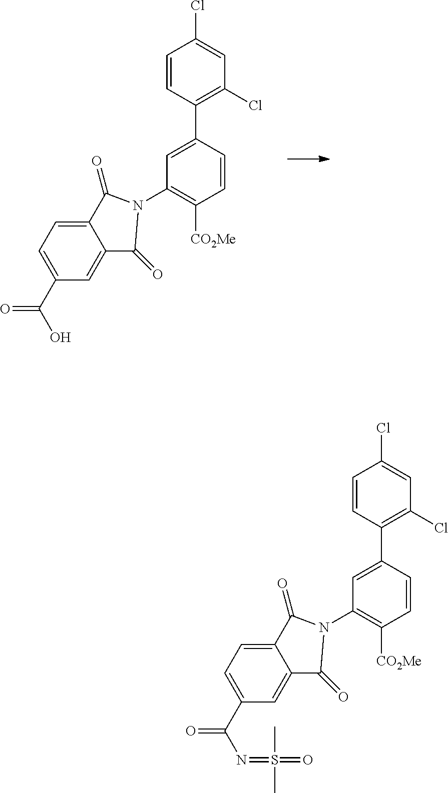 [SMILES: COC(=O)c1ccc(-c2ccc(Cl)cc2Cl)cc1N1C(=O)c2ccc(C(=O)O)cc2C1=O ; COC(=O)c1ccc(-c2ccc(Cl)cc2Cl)cc1N1C(=O)c2ccc(C(=O)N=S(C)(C)=O)cc2C1=O]

2-(2′,4′-dichloro-4-(methoxycarbonyl)-[1,1′-biphenyl]-3-yl)-1,3-dioxoisoindoline-5-carboxylic acid 0.150 g, 0.32 mmol) was dissolved in anhydrous DMF (4.5 mL) and treated with HATU (0.182 g, 0.48 mmol), iminodimethyl-λ⁶-sulfanone (0.045 g, 0.48 mmol) and diisopropylethylamine (167 μL, 0.96 mmol). The mixture was stirred at 35° C. for 3 h and then poured into water, extracted with ethyl acetate (3×). The combined organic layers were dried over sodium sulfate, filtered and concentrated to dryness. After drying under high vacuum, the crude product was used in the next step.

### Step 34.3 Preparation of 3-(2-carboxy-4-((dimethyl(oxo)-λ⁶-sulfanylidene)carbamoyl)benzamido)-2′,4′-dichloro-[1,1′-biphenyl]-4-carboxylic acid and 3-(2-carboxy-5-((dimethyl(oxo)-λ⁶-sulfanylidene)carbamoyl)benzamido)-2′,4′-dichloro-[1,1′-biphenyl]-4-carboxylic Acid

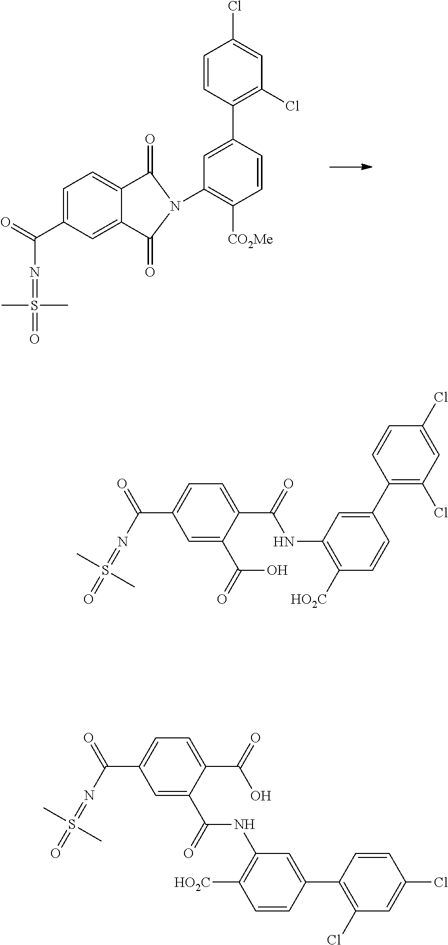 [SMILES: COC(=O)c1ccc(-c2ccc(Cl)cc2Cl)cc1N1C(=O)c2ccc(C(=O)N=S(C)(C)=O)cc2C1=O ; CS(C)(=O)=NC(=O)c1ccc(C(=O)Nc2cc(-c3ccc(Cl)cc3Cl)ccc2C(=O)O)c(C(=O)O)c1 ; CS(C)(=O)=NC(=O)c1ccc(C(=O)O)c(C(=O)Nc2cc(-c3ccc(Cl)cc3Cl)ccc2C(=O)O)c1]

The crude methyl 2′,4′-dichloro-3-(5-((dimethyl(oxo)-λ6-sulfanylidene)carbamoyl)-1,3-dioxoisoindolin-2-yl)-[1,1′-biphenyl]-4-carboxylate (0.175 g, 0.32 mmol) from the previous step was dissolved in a mixture of methanol (5 mL) and THF (2.5 mL) and treated with sodium hydroxide (1.3 mL, 2N, 2.6 mmol). The solution was stirred at room temperature for 1.5 h, then poured into 0.2N HCl and extracted with ethyl acetate (4×). The combined organic layers were washed with water, brine, dried over sodium sulfate, filtered and concentrated. The residue was purified on preparative HPLC to afford the two desired products, 3-(2-carboxy-4-((dimethyl(oxo)-λ6-sulfanylidene)carbamoyl)benzamido)-2′,4′-dichloro-[1,1′-biphenyl]-4-carboxylic acid and 3-(2-carboxy-5-((dimethyl(oxo)-λ6-sulfanylidene)carbamoyl)benzamido)-2′,4′-dichloro-[1,1′-biphenyl]-4-carboxylic acid. After preparative HPLC the target compound was selected as the isomer, which matched the NMR spectra.

1H NMR (250 MHz, DMSO-d6) δ ppm 3.51 (s, 6H) 7.30 (dt, J=8.19, 1.51 Hz, 1H) 7.49-7.54 (m, 1H) 7.56-7.63 (m, 1H) 7.82 (t, J=1.65 Hz, 1H) 7.95 (dd, J=7.91, 0.88 Hz, 1H) 8.11 (dd, J=8.13, 0.88 Hz, 1H) 8.16-8.24 (m, 2H) 8.63 (br. s., 1H) 11.62-11.71 (br. s., 1H)

### Example 35: 3-(2-carboxy-4-((dimethyl(oxo)-λ⁶-sulfanylidene)carbamoyl)benzamido)-2′,4′-dichloro-[1,1′-biphenyl]-4-carboxylic acid (GO-0003625)

3-(2-carboxy-4-((dimethyl(oxo)-λ⁶-sulfanylidene)carbamoyl)benzamido)-2′,4′-dichloro-[1,1′-biphenyl]-4-carboxylic acid was prepared at the Step 34.4 of Example 34. After preparative HPLC the target compound was selected as the isomer, which matched the NMR spectra.

1H NMR (250 MHz, DMSO-d6) δ ppm 3.52 (s, 6H) 7.30 (dt, J=8.30, 1.57 Hz, 1H) 7.48-7.54 (m, 1H) 7.56-7.63 (m, 1H) 7.77 (d, J=7.91 Hz, 1H) 7.82 (t, J=1.65 Hz, 1H) 8.11 (d, J=8.13 Hz, 1H) 8.22-8.28 (m, 1H) 8.38-8.48 (m, 1H) 8.59-8.68 (m, 1H) 11.63 (br. s., 1H)

### Example 36: 3-(2-carboxy-5-{[methyl(methylidene)oxo-λ⁶-sulfanyl]carbamoyl}benzamido)-3′,4′-difluoro-[1,1′-biphenyl]-4-carboxylic acid (GO-0003626)

3-(2-carboxy-5-{[methyl(methylidene)oxo-λ⁶-sulfanyl]carbamoyl}benzamido)-3′,4′-difluoro-[1,1′-biphenyl]-4-carboxylic acid was prepared in several steps.

### Step 36.1 Preparation of 2-(3′,4′-difluoro-4-(methoxycarbonyl)-[1,1′-biphenyl]-3-yl)-1,3-dioxoisoindoline-5-carboxylic acid

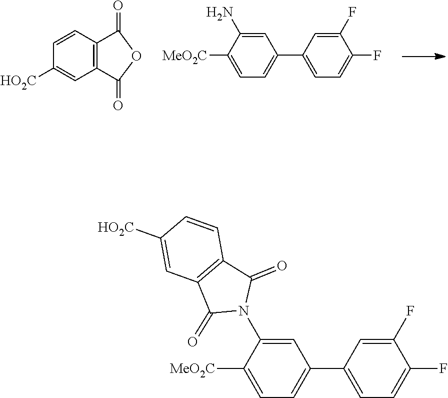 [SMILES: O=C(O)c1ccc2c(c1)C(=O)OC2=O ; COC(=O)c1ccc(-c2ccc(F)c(F)c2)cc1N ; COC(=O)c1ccc(-c2ccc(F)c(F)c2)cc1N1C(=O)c2ccc(C(=O)O)cc2C1=O]

A solution of 1,3-dioxo-1,3-dihydroisobenzofuran-5-carboxylic acid (2.25 g, 11.68 mmol) and methyl 3-amino-3′,4′-difluoro-[1,1′-biphenyl]-4-carboxylate (2.93 g, 11.1 mmol) in acetic acid (70 mL) was heated at 120° C. for 20 h. The cooled mixture was concentrated to ca. 30 mL at which point the product precipitated. The precipitate was filtered, rinsed with water followed by hexane, and then dried under high vacuum. The filtered solution was concentrated to dryness and the dried residue was purified by flash chromatography (silica gel, methanol (0-10%) in methylene chloride) to afford additional product which was combined with the precipitate to afford pure 2-(3′,4′-difluoro-4-(methoxycarbonyl)-[1,1′-biphenyl]-3-yl)-1,3-dioxoisoindoline-5-carboxylic acid (3.4 g, 71% yield).

### Step 36.2 Preparation of methyl 3-(5-((dimethyl(oxo)-λ⁶-sulfanylidene)carbamoyl)-1,3-dioxoisoindolin-2-yl)-3′,4′-difluoro-[1,1′-biphenyl]-4-carboxylate

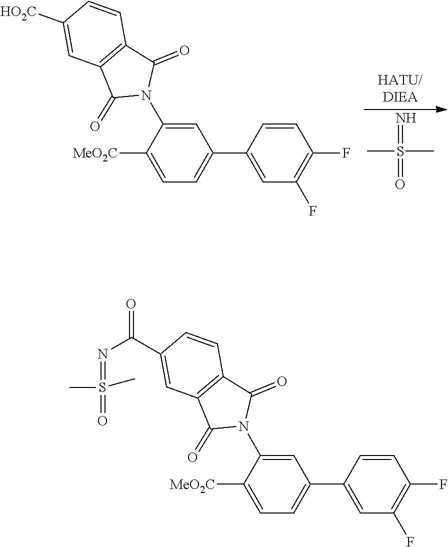

A solution of 2-(3′,4′-difluoro-4-(methoxycarbonyl)-[1,1′-biphenyl]-3-yl)-1,3-dioxoisoindoline-5-carboxylic acid (0.200 g, 457 mmol) in DMF (4.5 mL) was treated with HATU (0.182 g, 0.479 mmol), dimethyl sulfoximide (0.045 g, 0.479 mmol), and diisopropylethylamine (167 μL, 0.957 mmol) and stirred at 35° C. for 3 h. The reaction mixture was poured into water and extracted three times with ethyl acetate, the combined extracts were dried over sodium sulfate, filtered and evaporated to dryness to give a solid residue. The crude methyl 3-(5-((dimethyl(oxo)-λ6-sulfanylidene)carbamoyl)-1,3-dioxoisoindolin-2-yl)-3′,4′-difluoro-[1,1′-biphenyl]-4-carboxylate (0.240 g) was used without further purification in the next step.

### Step 36.3 Preparation of 3-(2-carboxy-5-((dimethyl(oxo)-λ⁶-sulfanylidene)carbamoyl)benzamido)-3′,4′-difluoro-[1,1′-biphenyl]-4-carboxylic acid and 3-(2-carboxy-4-((dimethyl(oxo)-λ⁶-sulfanylidene)carbamoyl)benzamido)-3′,4′-difluoro-[1,1′-biphenyl]-4-carboxylic Acid

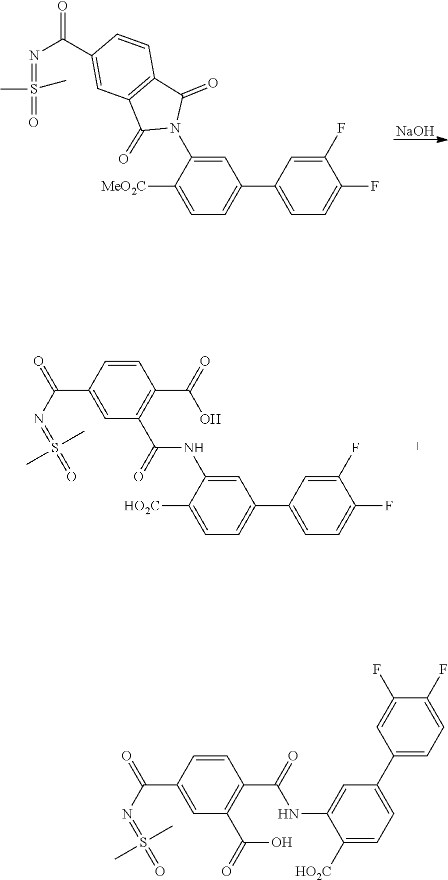

A solution of methyl 3-(5-((dimethyl(oxo)-λ6-sulfanylidene)carbamoyl)-1,3-dioxoisoindolin-2-yl)-3′,4′-difluoro-[1,1′-biphenyl]-4-carboxylate (0.230 g) in methanol (9 mL) and THF (18 mL) was treated with sodium hydroxide solution (1.5 mL, 2M aqueous) and the mixture was stirred at room temperature for 3.5 h. The reaction mixture was poured into HCl (0.2 M) and extracted with ethyl acetate (4×). The combined organics were washed with water, brine, dried over sodium sulfate. Concentration to dryness afforded a residue which was purified by preparative HPLC to afford pure 3-(2-carboxy-5-((dimethyl(oxo)-λ⁶-sulfanylidene)carbamoyl)benzamido)-3′,4′-difluoro-[1,1′-biphenyl]-4-carboxylic acid and 3-(2-carboxy-4-((dimethyl(oxo)-λ⁶-sulfanylidene)carbamoyl)-benzamido)-3′,4′-difluoro-[1,1′-biphenyl]-4-carboxylic acid. After preparative HPLC the target compound was selected as the isomer, which matched the NMR spectra.

1H NMR (250 MHz, DEUTERIUM OXIDE) δ ppm 3.51 (s, 6H) 7.52-7.67 (m, 3H) 7.76-7.88 (m, 1H) 7.96 (dd, J=8.13, 0.88 Hz, 1H) 8.10 (dd, J=8.24, 0.77 Hz, 1H) 8.20 (dd, J=8.13, 1.54 Hz, 1H) 8.24 (s, 1H) 8.83-8.88 (m, 1H)

### Example 37: 2-({3′,4′-difluoro-4-hydroxy-[1,1′-biphenyl]-3-yl}carbamoyl)-5-hydroxybenzene-1,4-dicarboxylic acid (GO-0003627)

2-({3′,4′-difluoro-4-hydroxy-[1,1′-biphenyl]-3-yl}carbamoyl)-5-hydroxybenzene-1,4-dicarboxylic acid was prepared at the Step 27.4 of Example 27. After preparative HPLC the target compound was selected as the isomer, which matched the NMR spectra.

NMR H1:

### Example 41: (GO-0003637) 3-(2-carboxy-4-{[methyl(methylidene)oxo-λ⁶-sulfanyl]carbamoyl}benzamido)-3′,4′-difluoro-[1,1′-biphenyl]-4-carboxylic acid

3-(2-carboxy-4-{[methyl(methylidene)oxo-λ⁶-sulfanyl]carbamoyl}benzamido)-3′,4′-difluoro-[1,1′-biphenyl]-4-carboxylic acid was prepared at the Step 36.3 of Example 36. After preparative HPLC the target compound was selected as the isomer, which matched the NMR spectra.

1H NMR (250 MHz, DEUTERIUM OXIDE) δ ppm 3.52 (s, 6H) 7.53-7.66 (m, 3H) 7.75-7.81 (m, 1H) 8.10 (dd, J=8.13, 1.32 Hz, 1H) 8.22-8.29 (m, 1H) 8.48 (s, 1H) 8.88 (br. s., 1H)

### Example 42: 2′,4′-dichloro-3-(5-((dimethyl(oxo)-λ⁶-sulfanylidene)carbamoyl)-2-(dimethylcarbamoyl)-4-hydroxybenzamido)-[1,1′-biphenyl]-4-carboxylic acid (GO-0003652) 2′,4′-dichloro-3-(5-((dimethyl(oxo)-λ6-sulfanylidene)carbamoyl)-2-(dimethylcarbamoyl)-4-hydroxybenzamido)-[1,1′-biphenyl]-4-carboxylic acid was prepared in several steps

### Step 42.1 Preparation of methyl 2′,4′-dichloro-3-(5-((dimethyl(oxo)-λ⁶-sulfanylidene)carbamoyl)-6-hydroxy-1,3-dioxoisoindolin-2-yl)-[1,1′-biphenyl]-4-carboxylate

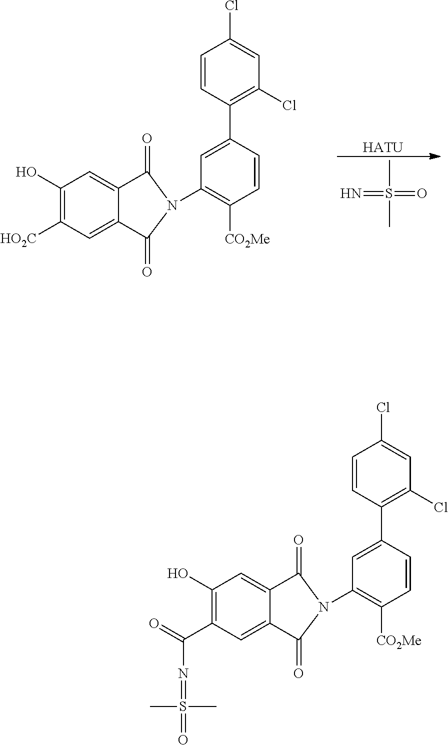

2-(2′,4′-dichloro-4-(methoxycarbonyl)-[1,1′-biphenyl]-3-yl)-6-hydroxy-1,3-dioxoisoindoline-5-carboxylic acid (0.20 g 0.41 mmol) was dissolved in DMF (5 mL) and treated with HATU (0.235 g, 0.617 mmol) and iminodimethyl-λ⁶-sulfanone (0.058 g, 0.617 mmol) and diisopropylethylamine (0.16 g, 1.23 mmol). The mixture was stirred at 35° C. overnight at which time it was poured into water and extracted three times with ethyl acetate. The combined organic layers were dried over sodium sulfate, filtered, evaporated to dryness. A second run of the same scale was carried out and the combined crude products were then purified by flash chromatography (silica gel, dichloromethane-methanol (0 to 20%)) to afford pure methyl 2′,4′-dichloro-3-(5-((dimethyl(oxo)-λ⁶-sulfanylidene)carbamoyl)-6-hydroxy-1,3-dioxoisoindolin-2-yl)-[1,1′-biphenyl]-4-carboxylate (0.217 g, 47% yield).

### Step 42.2 Preparation of methyl 2′,4′-dichloro-3-(4-((dimethyl(oxo)-λ⁶-sulfanylidene)carbamoyl)-2-(dimethylcarbamoyl)-5-hydroxybenzamido)-[1,1′-biphenyl]-4-carboxylate and methyl 2′,4′-dichloro-3-(5-((dimethyl(oxo)-λ⁶-sulfanylidene)carbamoyl)-2-(dimethylcarbamoyl)-4-hydroxybenzamido)-[1,1′-biphenyl]-4-carboxylate

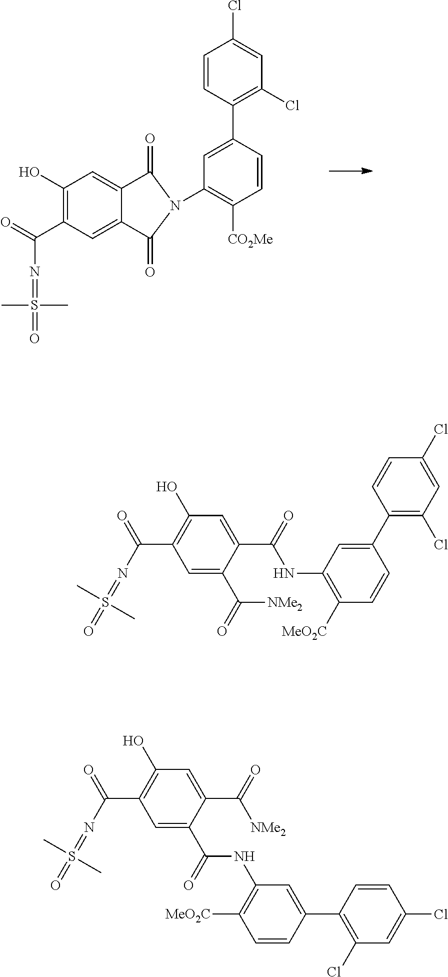 [SMILES: COC(=O)c1ccc(-c2ccc(Cl)cc2Cl)cc1N1C(=O)c2cc(O)c(C(=O)N=S(C)(C)=O)cc2C1=O ; COC(=O)c1ccc(-c2ccc(Cl)cc2Cl)cc1NC(=O)c1cc(O)c(C(=O)N=S(C)(C)=O)cc1C(=O)N(C)C ; COC(=O)c1ccc(-c2ccc(Cl)cc2Cl)cc1NC(=O)c1cc(C(=O)N=S(C)(C)=O)c(O)cc1C(=O)N(C)C]

Methyl 2′,4′-dichloro-3-(5-((dimethyl(oxo)-λ⁶-sulfanylidene)carbamoyl)-6-hydroxy-1,3-dioxoisoindolin-2-yl)-[1,1′-biphenyl]-4-carboxylate (0.21 g, 0.374 mmol) was dissolved in THF (1.2 mL) and treated at room temperature with dimethylamine (2.8 mL, 2M in THF, 5.6 mmol) for 70 min. The reaction mixture was diluted with THF and evaporated to dryness and the crude product mixture (0.236 g) was used in the next step.

### Step 42.3 Preparation of 2′,4′-dichloro-3-(4-((dimethyl(oxo)-λ⁶-sulfanylidene)carbamoyl)-2-(dimethyl-carbamoyl)-5-hydroxybenzamido)-[1,1′-biphenyl]-4-carboxylic acid and 2′,4′-dichloro-3-(5-((dimethyl(oxo)-λ6-sulfanylidene)carbamoyl)-2-(dimethylcarbamoyl)-4-hydroxybenzamido)-[1,1′-biphenyl]-4-carboxylic Acid

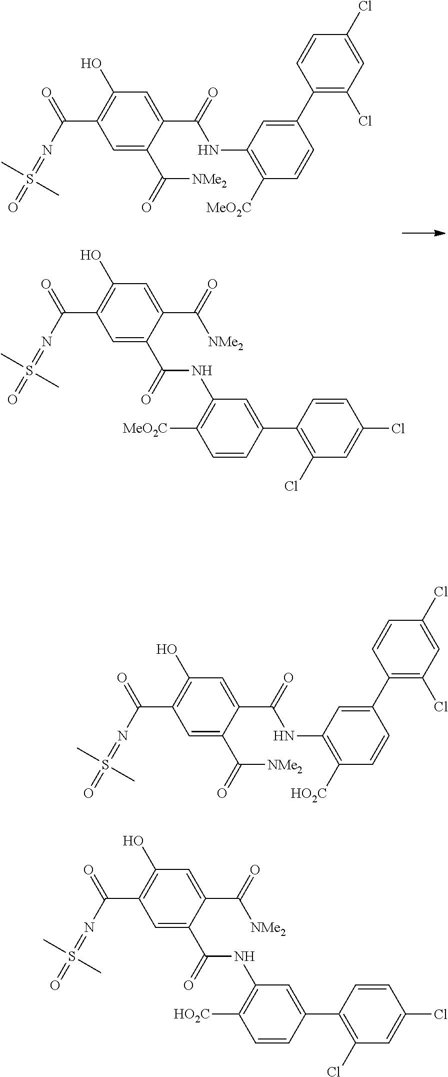 [SMILES: COC(=O)c1ccc(-c2ccc(Cl)cc2Cl)cc1NC(=O)c1cc(O)c(C(=O)N=S(C)(C)=O)cc1C(=O)N(C)C ; COC(=O)c1ccc(-c2ccc(Cl)cc2Cl)cc1NC(=O)c1cc(C(=O)N=S(C)(C)=O)c(O)cc1C(=O)N(C)C ; CN(C)C(=O)c1cc(C(=O)N=S(C)(C)=O)c(O)cc1C(=O)Nc1cc(-c2ccc(Cl)cc2Cl)ccc1C(=O)O ; CN(C)C(=O)c1cc(O)c(C(=O)N=S(C)(C)=O)cc1C(=O)Nc1cc(-c2ccc(Cl)cc2Cl)ccc1C(=O)O]

The crude product mixture from the previous step (0.236 g, 0.39 mmol) was dissolved in a mixture of THE (2 mL) and methanol (2 mL) and treated with sodium hydroxide (2M, 0.98 mL, 1.96 mmol) at room temperature for 30 min. The reaction mixture was poured into 0.2N HCl and extracted with diethyl ether (2×) followed by ethyl acetate (2×). The combined organic layers were washed with water, brine, then dried over sodium sulfate, filtered, concentrated. The solid was sent for final separation and purification of the two products by HPLC to afford pure and 2′,4′-dichloro-3-(5-((dimethyl(oxo)-λ6-sulfanylidene)carbamoyl)-2-(dimethylcarbamoyl)-4-hydroxybenzamido)-[1,1′-biphenyl]-4-carboxylic acid. After preparative HPLC the target compound was selected as the isomer, which matched the NMR spectra.

1H NMR (250 MHz, DMSO-d6) δ ppm 2.78 (s, 3H) 2.92 (s, 3H) 3.61-3.66 (m, 6H) 6.87 (s, 1H) 7.27 (dd, J=8.24, 1.87 Hz, 1H) 7.47-7.54 (m, 1H) 7.56 (d, J=1.98 Hz, 1H) 7.81 (d, J=1.98 Hz, 1H) 8.13 (d, J=8.13 Hz, 1H) 8.52 (s, 1H) 8.59-8.63 (m, 1H)

### Example 43: 4-({4-carboxy-2′,4′-dichloro-[1,1′-biphenyl]-3-yl}carbamoyl)-6-hydroxybenzene-1,3-dicarboxylic acid (GO-0003653)

4-({4-carboxy-2′,4′-dichloro-[1,1′-biphenyl]-3-yl}carbamoyl)-6-hydroxybenzene-1,3-dicarboxylic acid was prepared in one step.

### Step 43.1 Preparation of 2-((4-carboxy-2′,4′-dichloro-[1,1′-biphenyl]-3-yl)carbamoyl)-5-hydroxyterephthalic acid, 4-((4-carboxy-2′,4′-dichloro-[1,1′-biphenyl]-3-yl)carbamoyl)-6-hydroxyisophthalic acid and 2-(4-carboxy-2′,4′-dichloro-[1,1′-biphenyl]-3-yl)-6-hydroxy-1,3-dioxoisoindoline-5-carboxylic Acid

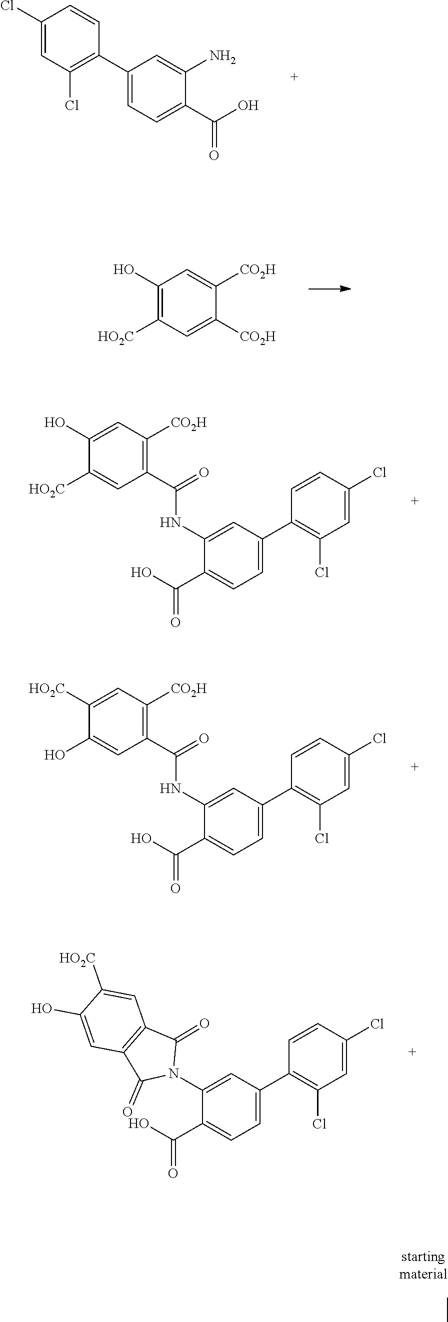 [SMILES: Nc1cc(-c2ccc(Cl)cc2Cl)ccc1C(=O)O ; O=C(O)c1cc(C(=O)O)c(C(=O)O)cc1O ; O=C(O)c1cc(C(=O)Nc2cc(-c3ccc(Cl)cc3Cl)ccc2C(=O)O)c(C(=O)O)cc1O ; O=C(O)c1cc(C(=O)O)c(C(=O)Nc2cc(-c3ccc(Cl)cc3Cl)ccc2C(=O)O)cc1O ; O=C(O)c1cc2c(cc1O)C(=O)N(c1cc(-c3ccc(Cl)cc3Cl)ccc1C(=O)O)C2=O]

3-amino-2′,4′-dichloro-[1,1′-biphenyl]-4-carboxylic acid (100 mg, 1 equiv) and 5-hydroxybenzene-1,2,4-tricarboxylic acid (340 mg, 4.3 equiv) were dissolved in isobutyric acid (12 mL) and heated in a microwave reactor at 140° C. for 10 min. The solvent was removed under reduced pressure and the residue was purified by preparative HPLC to afford pure 2-((4-carboxy-2′,4′-dichloro-[1,1′-biphenyl]-3-yl)carbamoyl)-5-hydroxyterephthalic acid, 4-((4-carboxy-2′,4′-dichloro-[1,1′-biphenyl]-3-yl)carbamoyl)-6-hydroxyisophthalic acid and 2-(4-carboxy-2′,4′-dichloro-[1,1′-biphenyl]-3-yl)-6-hydroxy-1,3-dioxoisoindoline-5-carboxylic acid as well as recovered starting material. After preparative HPLC the target compound was selected as the isomer, which matched the NMR spectra.

1H NMR (250 MHz, DMSO-d6) δ ppm 7.19 (s, 1H) 7.28 (dd, J=8.19, 1.26 Hz, 1H) 7.46-7.53 (m, 1H) 7.58 (dd, J=8.35, 1.87 Hz, 1H) 7.79 (d, J=1.76 Hz, 1H) 8.09 (d, J=8.13 Hz, 1H) 8.36 (s, 1H) 8.60 (s, 1H)

Additional Material
3-amino-2′,4′-dichloro-[1,1′-biphenyl]-4-carboxylic acid could be prepared according General procedure #1, using 3,5-Dichlorophenylboronic acid, 2-amino-4-bromobenzoic acid, Pd(PPh₃)₄ and potassium carbonate
Synthesis of 5-hydroxybenzene-1,2,4-tricarboxylic acid described as Intermediate 5b
Commercially available starting materials

| | CAS or MLD | Catalog number, |
|---|---|---|
| Compound | number | provider |
| 2-Amino-4-bromobenzoic acid | 20776-50-5 | 664863, Sigma Aldrich |
| 3,5-Dichlorophenylboronic acid | 67492-50-6 | 445207, Sigma-Aldrich |
| Pd(PPh3)4: | 14221-01-3 | 216666, Sigma-Aldrich |
| tetrakis(triphenylphosphine)paliadium(0) | | |
| potassium carbonate | 584-08-7 | 590681, Sigma-Aldrich |

### Example 44: (GO-0003654) 2-({4-carboxy-2′,4′-dichloro-[1,1′-biphenyl]-3-yl}carbamoyl)-5-hydroxybenzene-1,4-dicarboxylic acid

2-({4-carboxy-2′,4′-dichloro-[1,1′-biphenyl]-3-yl}carbamoyl)-5-hydroxybenzene-1,4-dicarboxylic acid was prepared at the Step 43.1 of Example 43. After preparative HPLC the target compound was selected as the isomer, which matched the NMR spectra.

1H NMR (250 MHz, DMSO-d6) δ ppm 7.23 (s, 1H) 7.28 (dd, J=8.19, 0.93 Hz, 1H) 7.46-7.52 (m, 1H) 7.58 (dd, J=8.35, 1.43 Hz, 1H) 7.80 (d, J=1.65 Hz, 1H) 8.11 (d, J=8.24 Hz, 1H) 8.18 (s, 1H) 8.63 (s, 1H)

### Example 45: 4-({4-carboxy-3′,4′-difluoro-[1,1′-biphenyl]-3-yl}carbamoyl)-6-chlorobenzene-1,3-dicarboxylic acid (GO-0003655)

4-({4-carboxy-3′,4′-difluoro-[1,1′-biphenyl]-3-yl}carbamoyl)-6-chlorobenzene-1,3-dicarboxylic acid was prepared in several steps.

### Step 45.1 Preparation of 2-(4-carboxy-3′,4′-difluoro-[1,1′-biphenyl]-3-yl)-6-chloro-1,3-dioxoisoindoline-5-carboxylic Acid

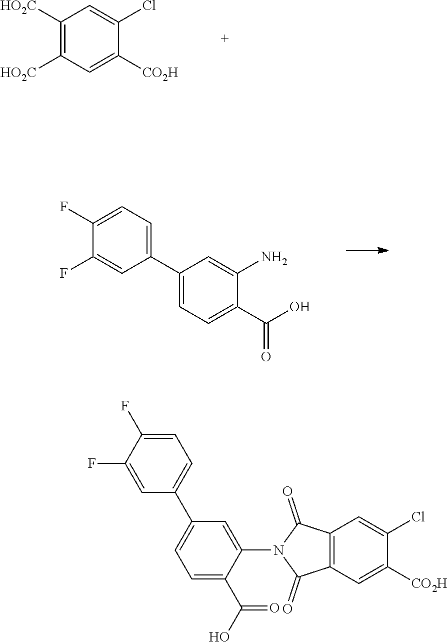 [SMILES: O=C(O)c1cc(C(=O)O)c(C(=O)O)cc1Cl ; Nc1cc(-c2ccc(F)c(F)c2)ccc1C(=O)O ; O=C(O)c1cc2c(cc1Cl)C(=O)N(c1cc(-c3ccc(F)c(F)c3)ccc1C(=O)O)C2=O]

A mixture of 5-chlorobenzene-1,2,4-tricarboxylic acid (0.0504 g, 0.2 mmol) and 3-amino-3′,4′-difluoro-[1,1′-biphenyl]-4-carboxylic acid (0.052 g, 0.2 mmol) in isobutyric acid (2.5 mL) was heated in a microwave reactor at 175° C. for 4 h. Removal of the solvent under reduced pressure followed by preparative HPLC chromatography afforded pure 2-(4-carboxy-3′,4′-difluoro-[1,1′-biphenyl]-3-yl)-6-chloro-1,3-dioxoisoindoline-5-carboxylic acid.

### Step 45.2 Preparation of 2-((4-carboxy-3′,4′-difluoro-[1,1′-biphenyl]-3-yl)carbamoyl)-5-chloroterephthalic acid and 4-((4-carboxy-3′,4′-difluoro-[1,1′-biphenyl]-3-yl)carbamoyl)-6-chloroisophthalic acid

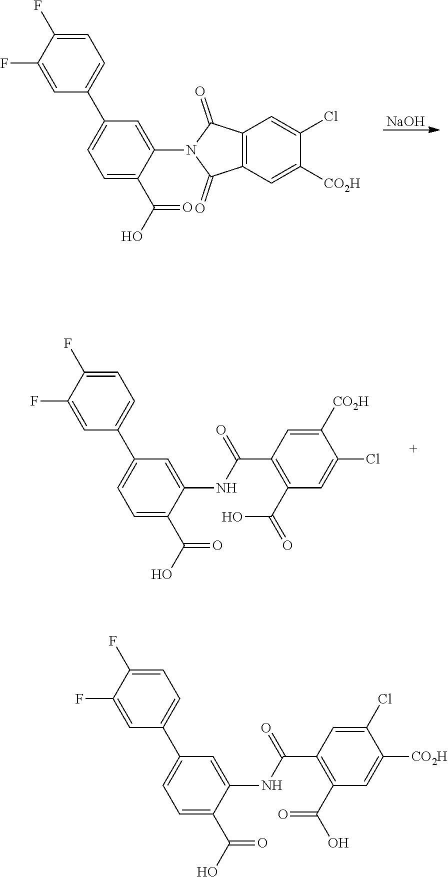

2-(4-carboxy-3′,4′-difluoro-[1,1′-biphenyl]-3-yl)-6-chloro-1,3-dioxoisoindoline-5-carboxylic acid. (0.035 g, 0.0764 mmol) was dissolved in THF (1 mL) and methanol (1 mL) and treated with sodium hydroxide (0.38 mL, 2M aqueous, 10 equiv). After stirring at room temperature for 1 h the reaction mixture was poured into HCl (0.2 M) and extracted with ethyl acetate (4×). The combined organics were washed with water, brine, dried over sodium sulfate. Concentration to dryness afforded a residue which was purified by preparative HPLC to afford pure 2-((4-carboxy-3′,4′-difluoro-[1,1′-biphenyl]-3-yl)carbamoyl)-5-chloroterephthalic acid and 4-((4-carboxy-3′,4′-difluoro-[1,1′-biphenyl]-3-yl)carbamoyl)-6-chloroisophthalic acid. After preparative HPLC the target compound was selected as the isomer, which matched the NMR spectra.

1H NMR (250 MHz, DEUTERIUM OXIDE) δ ppm 7.53-7.66 (m, 3H) 7.74-7.86 (m, 1H) 7.92 (s, 1H) 8.09 (dd, J=8.13, 1.10 Hz, 1H) 8.27 (s, 1H) 8.75 (s, 1H)

### Example 46: 2-({4-carboxy-3′,4′-difluoro-[1,1′-biphenyl]-3-yl}carbamoyl)-5-chlorobenzene-1,4-dicarboxylic acid (GO-0003656)

2-({4-carboxy-3′,4′-difluoro-[1,1′-biphenyl]-3-yl}carbamoyl)-5-chlorobenzene-1,4-dicarboxylic acid was prepared at the Step 45.2 of Example 45. After preparative HPLC the target compound was selected as the isomer, which matched the NMR spectra.

1H NMR (250 MHz, DMSO-d6) δ ppm 7.52-7.66 (m, 3H) 7.75-7.86 (m, 1H) 7.95 (d, J=1.32 Hz, 1H) 8.05-8.13 (m, 2H) 8.77 (s, 1H)

### Example 49: 2-[(2′,4′-dichloro-4-{[2-(dimethylamino)ethoxy]carbonyl}-[1,1′-biphenyl]-3-yl)carbamoyl]-5-{[dimethyl(oxo)-λ⁶-sulfanylidene]carbamoyl}benzoic acid (GO-0003714)

2-[(2′,4′-dichloro-4-{[2-(dimethylamino)ethoxy]carbonyl}-[1,1′-biphenyl]-3-yl)carbamoyl]-5-{[dimethyl(oxo)-λ⁶-sulfanylidene]carbamoyl}benzoic acid was prepared at the Step 50.3 of Example 50. After preparative HPLC the target compound was selected as the isomer, which didn't match the NMR spectra of undesirable isomer.

### Example 50: 2-[(2′,4′-dichloro-4-{[2-(dimethylamino)ethoxy]carbonyl}-[1,1′-biphenyl]-3-yl)carbamoyl]-4-{[methyl(methylidene)oxo-λ⁶-sulfanyl]carbamoyl}benzoic acid (GO-0003713)

2-[(2′,4′-dichloro-4-{[2-(dimethylamino)ethoxy]carbonyl}-[1,1′-biphenyl]-3-yl)carbamoyl]-4-{[methyl(methylidene)oxo-λ⁶-sulfanyl]carbamoyl}benzoic acid was prepared in several steps.

### Step 50.1 Preparation of N-(dimethyl(oxo)-λ⁶-sulfanylidene)-1,3-dioxo-1,3-dihydroisobenzofuran-5-carboxamide

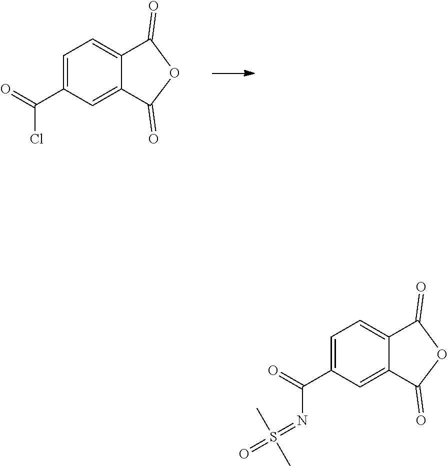 [SMILES: O=C(Cl)c1ccc2c(c1)C(=O)OC2=O ; CS(C)(=O)=NC(=O)c1ccc2c(c1)C(=O)OC2=O]

1,3-dioxo-1,3-dihydroisobenzofuran-5-carbonyl chloride (0.105 g, 0.5 mmol) was dissolved in dichloromethane (2 mL) and treated with iminodimethyl-λ⁶-sulfanone (0.049 g, 0.525 mmol) at 0° C. followed by triethylamine (84 μL, 0.6 mmol). The resulting reaction mixture was allowed to stir at room temperature for 2.5 h, after which time it was poured into water and extracted with 2-methyl tetrahydrofuran (3×), dried over sodium sulfate, filtered, concentrated and dried under high vacuum to afford crude N-(dimethyl(oxo)-λ6-sulfanylidene)-1,3-dioxo-1,3-dihydroisobenzofuran-5-carboxamide which was used with no further purification.

### Step 50.2 Preparation of 3-(dimethylamino)propyl 3-amino-2′,4′-dichloro-[1,1′-biphenyl]-4-carboxylate

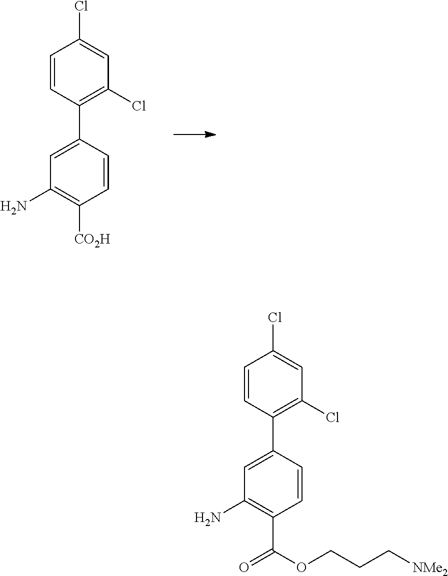 [SMILES: Nc1cc(-c2ccc(Cl)cc2Cl)ccc1C(=O)O ; CN(C)CCCOC(=O)c1ccc(-c2ccc(Cl)cc2Cl)cc1N]

3-amino-2′,4′-dichloro-[1,1′-biphenyl]-4-carboxylic acid (0.300 g, 1.2 mmol) was dissolved in anhydrous DMF (12 mL) and treated with HATU (0.687 g, 1.8 mmol), 3-(dimethylamino)propan-1-ol (1.21 mL, 12 mmol) and diisopropylethylamine (0.63 mL, 3 mmol). The mixture was stirred overnight at room temperature, and then poured into water, extracted with ethyl acetate (3×). The combined organic layers were dried over sodium sulfate, filtered and concentrated to dryness, and purified by flash chromatography (silica gel, dichloromethane-methanol (0 to 15%)) to afford 3-(dimethylamino)propyl 3-amino-2′,4′-dichloro-[1,1′-biphenyl]-4-carboxylate (0.275 g, 71% yield).

### Step 50.3 Preparation of 2-((2′,4′-dichloro-4-((3-(dimethylamino)propoxy)carbonyl)-[1,1′-biphenyl]-3-yl)carbamoyl)-5-((dimethyl(oxo)-λ⁶-sulfanylidene)carbamoyl)benzoic acid and 2-((2′,4′-dichloro-4-((3-(dimethylamino)propoxy)carbonyl)-[1,1′-biphenyl]-3-yl)carbamoyl)-4-((dimethyl(oxo)-λ⁶-sulfanylidene)carbamoyl)benzoic acid

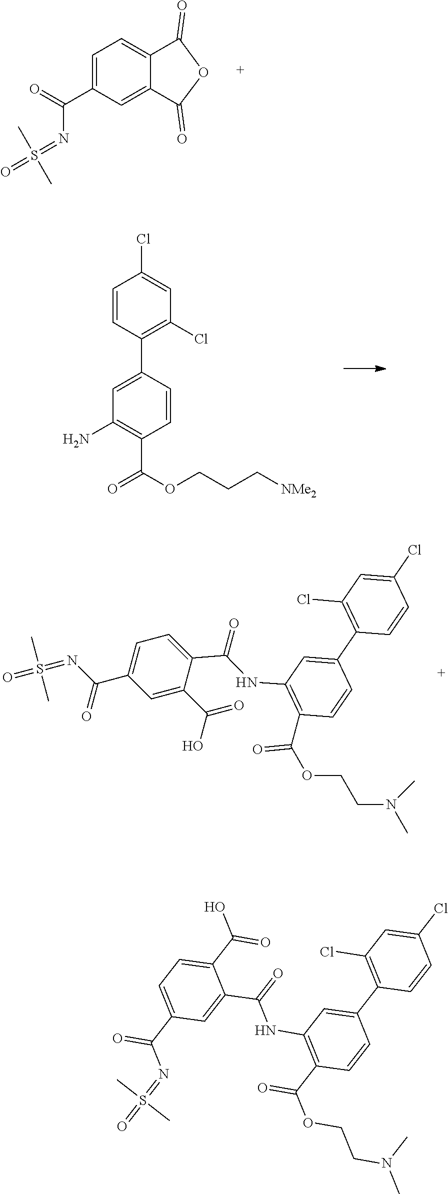

The crude N-(dimethyl (oxo)-λ6-sulfanylidene)-1,3-dioxo-1,3-dihydroisobenzofuran-5-carboxamide (0.134 g, 0.5 mmol) (from step 1 above) and 3-(dimethylamino)propyl 3-amino-2′,4′-dichloro-[1,1′-biphenyl]-4-carboxylate (0.050 g, 0.14 mmol) from step 2 above were dissolved in acetic acid (3 mL) and THF (4 mL) and heated to 45° C. for 1 h. The solvents were removed under high vacuum and the residue was purified by HPLC to afford the pure isomers, 2-((2′,4′-dichloro-4-((3-(dimethylamino)propoxy)carbonyl)-[1,1′-biphenyl]-3-yl)carbamoyl)-5-((dimethyl(oxo)-λ6-sulfanylidene)carbamoyl)benzoic acid and 2-((2′,4′-dichloro-4-((3-(dimethylamino)propoxy)carbonyl)-[1,1′-biphenyl]-3-yl)carbamoyl)-4-((dimethyl(oxo)-λ6-sulfanylidene)carbamoyl)benzoic acid. After preparative HPLC the target compound was selected as the isomer, which matched the NMR spectra.

### Example 51: 4-[(2′,4′-dichloro-4-{[3-(dimethylamino)propoxy]carbonyl}-[1,1′-biphenyl]-3-yl)carbamoyl]-6-hydroxybenzene-1,3-dicarboxylic acid (GO-0003722)

4-[(2′,4′-dichloro-4-{[3-(dimethylamino)propoxy]carbonyl}-[1,1′-biphenyl]-3-yl)carbamoyl]-6-hydroxybenzene-1,3-dicarboxylic acid was prepared in several steps.

### Step 51.1 Preparation of 3-(dimethylamino)propyl 3-amino-2′,4′-dichloro-[1,1′-biphenyl]-4-carboxylate

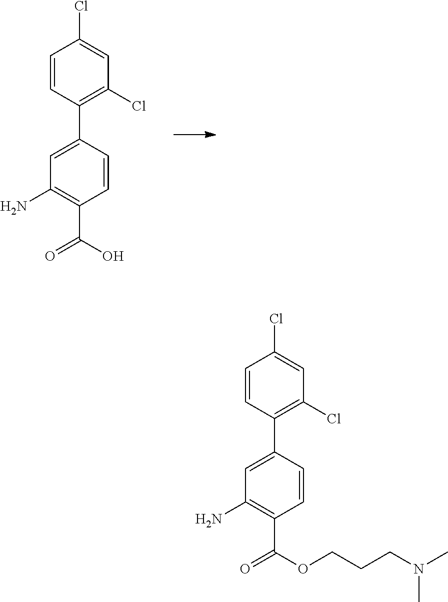 [SMILES: Nc1cc(-c2ccc(Cl)cc2Cl)ccc1C(=O)O ; CN(C)CCCOC(=O)c1ccc(-c2ccc(Cl)cc2Cl)cc1N]

3-amino-2′,4′-dichloro-[1,1′-biphenyl]-4-carboxylic acid (0.200 g) was dissolved in DMF (8 mL) and treated with HATU (0.404 g), 3-(dimethylamino)propan-1-ol (0.84 mL) and diisopropylethylamine (0.38 mL) at room temperature overnight. The reaction mixture was poured into water, extracted with ethyl acetate (3×), dried and concentrated to afford the desired ester, 3-(dimethylamino)propyl 3-amino-2′,4′-dichloro-[1,1′-biphenyl]-4-carboxylate (0.237 g, 91% yield) which was used without further purification. Step 51.2 Preparation of 4-((2′,4′-dichloro-4-((3-(dimethylamino)propoxy)carbonyl)-[1,1′-biphenyl]-3-yl)carbamoyl)-6-hydroxyisophthalic acid and 2-((2′,4′-dichloro-4-((3-(dimethylamino)propoxy)carbonyl)-[1,1′-biphenyl]-3-yl)carbamoyl)-5-hydroxyterephthalic acid

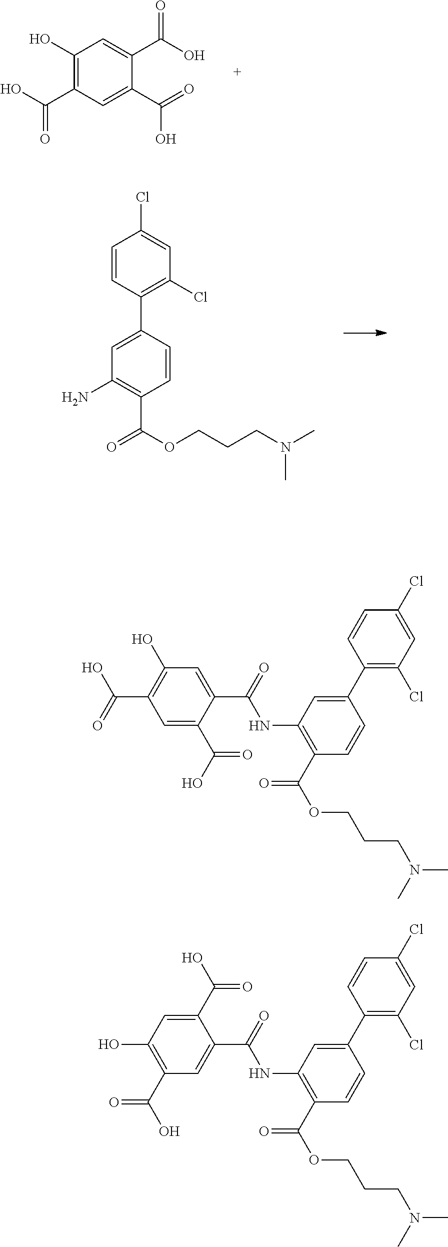

Using General Procedures 5 and 6, 5-hydroxybenzene-1,2,4-tricarboxylic acid (0.123 g) and 3-(dimethyl-amino)propyl 3-amino-2′,4′-dichloro-[1,1′-biphenyl]-4-carboxylate (0.100 g) afforded a mixture of 4-((2′,4′-dichloro-4-((3-(dimethylamino)propoxy)carbonyl)-[1,1′-biphenyl]-3-yl)carbamoyl)-6-hydroxyisophthalic acid and 2-((2′,4′-dichloro-4-((3-(dimethylamino)-propoxy)carbonyl)-[1,1′-biphenyl]-3-yl)carbamoyl)-5-hydroxyterephthalic acid which were separated by preparative HPLC. After preparative HPLC the target compound was selected as the isomer, which matched the NMR spectra.

1H NMR (250 MHz, DMSO-d6) δ 10.93 (d, J=4.6 Hz, 1H), 9.45 (s, 1H), 8.35 (d, J=3.0 Hz, 2H), 8.07 (dd, J=8.2, 3.2 Hz, 1H), 7.82 (d, J=2.1 Hz, 1H), 7.60 (dd, J=8.3, 2.2 Hz, 1H), 7.49 (d, J=8.3 Hz, 1H), 7.34 (dd, J=8.2, 1.8 Hz, 1H), 7.08 (d, J=12.7 Hz, 1H), 4.34 (t, J=6.0 Hz, 2H), 3.21 (s, 2H), 2.79 (d, J=4.6 Hz, 6H), 2.09 (s, 2H).

### Example 52: 4-{[2-carboxy-5-(4,4-dimethylcyclohexyl)phenyl]carbamoyl}-6-hydroxybenzene-1,3-dicarboxylic acid (GO-0003739)

4-{[2-carboxy-5-(4,4-dimethylcyclohexyl)phenyl]carbamoyl}-6-hydroxybenzene-1,3-dicarboxylic acid was prepared in one step. Step 52.1 Preparation of 4-((2-carboxy-5-(4,4-dimethylcyclohexyl)phenyl)carbamoyl)-6-hydroxyisophthalic acid and 2-((2-carboxy-5-(4,4-dimethylcyclohexyl)phenyl)carbamoyl)-5-hydroxyterephthalic acid

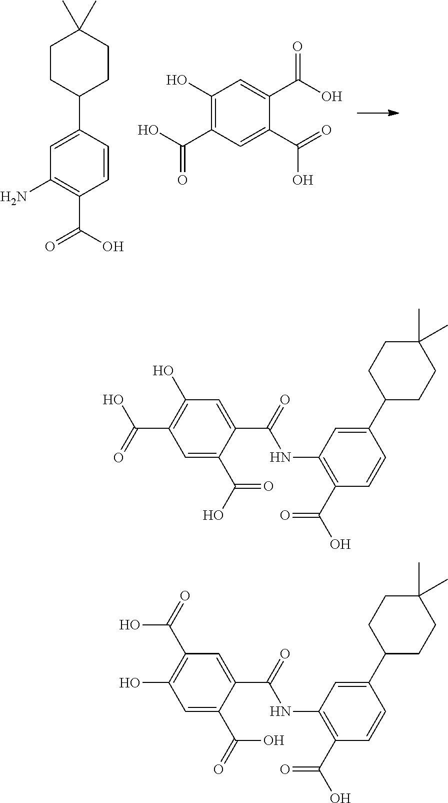 [SMILES: CC1(C)CCC(c2ccc(C(=O)O)c(N)c2)CC1 ; O=C(O)c1cc(C(=O)O)c(C(=O)O)cc1O ; CC1(C)CCC(c2ccc(C(=O)O)c(NC(=O)c3cc(O)c(C(=O)O)cc3C(=O)O)c2)CC1 ; CC1(C)CCC(c2ccc(C(=O)O)c(NC(=O)c3cc(C(=O)O)c(O)cc3C(=O)O)c2)CC1]

Using General Procedures 5 and 6, 2-amino-4-(4,4-dimethylcyclohexyl)benzoic acid (0.050 g) was reacted with 5-hydroxybenzene-1,2,4-tricarboxylic acid (0.090 g) to afford a mixture of 4-((2-carboxy-5-(4,4-dimethylpiperidin-1-yl)phenyl)carbamoyl)-6-hydroxyisophthalic acid 4-((2-carboxy-5-(4,4-dimethylcyclohexyl)-phenyl)carbamoyl)-6-hydroxyisophthalic acid and 2-((2-carboxy-5-(4,4-dimethylcyclohexyl)phenyl)carbamoyl)-5-hydroxyterephthalic acid which were separated and purified by preparative HPLC. After preparative HPLC the target compound was selected as the isomer, which didn't match the NMR spectra of undesirable isomer.

### Example 53 4-{[2-carboxy-5-(4,4-dimethylpiperidin-1-yl)phenyl]carbamoyl}-6-hydroxybenzene-1,3-dicarboxylic acid (GO-0003740)

4-{[2-carboxy-5-(4,4-dimethylpiperidin-1-yl)phenyl]carbamoyl}-6-hydroxybenzene-1,3-dicarboxylic acid was prepared in one step.

### Step 53.1 Preparation of 4-((2-carboxy-5-(4,4-dimethylpiperidin-1-yl)phenyl)carbamoyl)-6-hydroxyisophthalic acid and 2-((2-carboxy-5-(4,4-dimethylpiperidin-1-yl)phenyl)carbamoyl)-5-hydroxyterephthalic acid

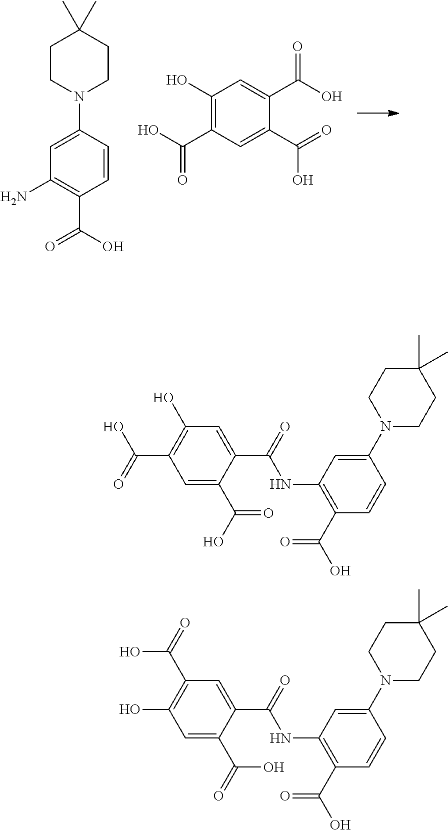 [SMILES: CC1(C)CCN(c2ccc(C(=O)O)c(N)c2)CC1 ; O=C(O)c1cc(C(=O)O)c(C(=O)O)cc1O ; CC1(C)CCN(c2ccc(C(=O)O)c(NC(=O)c3cc(O)c(C(=O)O)cc3C(=O)O)c2)CC1 ; CC1(C)CCN(c2ccc(C(=O)O)c(NC(=O)c3cc(C(=O)O)c(O)cc3C(=O)O)c2)CC1]

Using General Procedures 5 and 6, 2-amino-4-(4,4-dimethylpiperidin-1-yl)benzoic acid ((0.050 g) was reacted with 5-hydroxybenzene-1,2,4-tricarboxylic acid (0.090 g) to afford a mixture of 4-((2-carboxy-5-(4,4-dimethylpiperidin-1-yl)phenyl)carbamoyl)-6-hydroxyisophthalic acid and 2-((2-carboxy-5-(4,4-dimethylpiperidin-1-yl)phenyl)carbamoyl)-5-hydroxyterephthalic acid which were separated and purified by preparative HPLC. After preparative HPLC the target compound was selected as the isomer, which didn't match the NMR spectra of undesirable isomer.

### Example 54: 2-[(2′,4′-dichloro-4-{[3-(dimethylamino)propoxy]carbonyl}-[1,1′-biphenyl]-3-yl)carbamoyl]-5-hydroxybenzene-1,4-dicarboxylic acid (GO-0003744)

2-[(2′,4′-dichloro-4-{[3-(dimethylamino)propoxy]carbonyl}-[1,1′-biphenyl]-3-yl)carbamoyl]-5-hydroxybenzene-1,4-dicarboxylic acid was prepared at the Step 51.2 of Example 51. After preparative HPLC the target compound was selected as the isomer, which didn't match the NMR spectra of undesirable isomer.

### Example 55 4-{[2′,4′-dichloro-4-(ethoxycarbonyl)-[1,1′-biphenyl]-3-yl]carbamoyl}-6-hydroxybenzene-1,3-dicarboxylic acid (GO-0003745)

4-{[2′,4′-dichloro-4-(ethoxycarbonyl)-[1,1′-biphenyl]-3-yl]carbamoyl}-6-hydroxybenzene-1,3-dicarboxylic acid was prepared in one step.

### Step 55.1 Preparation of 4-((2′,4′-dichloro-4-(ethoxycarbonyl)-[1,1′-biphenyl]-3-yl)carbamoyl)-6-hydroxyisophthalic acid and 2-((2′,4′-dichloro-4-(ethoxycarbonyl)-[1,1′-biphenyl]-3-yl)carbamoyl)-5-hydroxyterephthalic acid

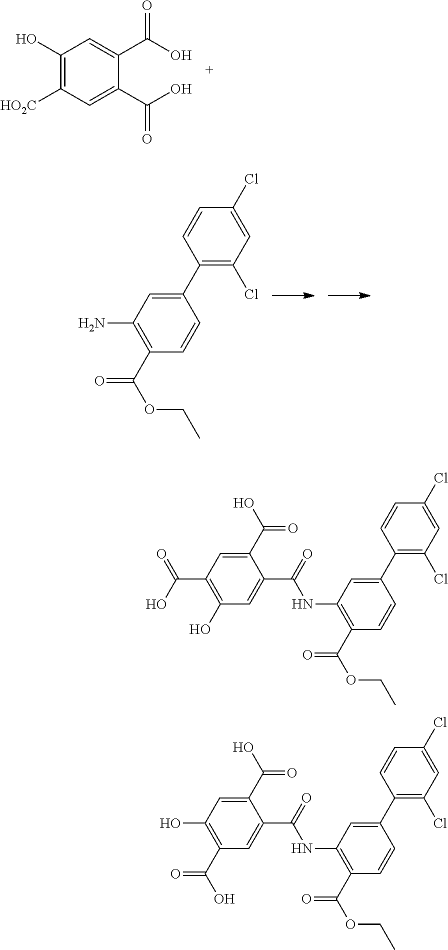 [SMILES: O=C(O)c1cc(C(=O)O)c(C(=O)O)cc1O ; CCOC(=O)c1ccc(-c2ccc(Cl)cc2Cl)cc1N ; CCOC(=O)c1ccc(-c2ccc(Cl)cc2Cl)cc1NC(=O)c1cc(O)c(C(=O)O)cc1C(=O)O ; CCOC(=O)c1ccc(-c2ccc(Cl)cc2Cl)cc1NC(=O)c1cc(C(=O)O)c(O)cc1C(=O)O]

5-hydroxybenzene-1,2,4-tricarboxylic acid (0.160 g, 2 equiv) was dissolved in isobutyric acid (6 mL) and heated at 150° C. for 3 h. The cooled reaction mixture was added to a solution of ethyl 3-amino-2′,4′-dichloro-[1,1′-biphenyl]-4-carboxylate (0.109 g, 1 equiv) in acetic acid (6 mL) and stirred at 45° C. for 40 min. The solvent was removed at <35° C. under reduced pressure, and the residue was separated by preparative HPLC to afford 4-((2′,4′-dichloro-4-(ethoxycarbonyl)-[1,1′-biphenyl]-3-yl)carbamoyl)-6-hydroxyisophthalic acid and 2-((2′,4′-dichloro-4-(ethoxycarbonyl)-[1,1′-biphenyl]-3-yl)carbamoyl)-5-hydroxyterephthalic acid. After preparative HPLC the target compound was selected as the isomer, which matched the NMR spectra.

1H NMR (250 MHz, DMSO-d6) δ 10.96 (s, 1H), 8.38 (d, J=5.5 Hz, 2H), 8.04 (d, J=7.9 Hz, 1H), 7.81 (d, J=2.0 Hz, 1H), 7.59 (dd, J=7.8, 2.4 Hz, 1H), 7.49 (d, J=8.4 Hz, 1H), 7.37-7.28 (m, 1H), 7.15 (s, 1H), 4.31 (q, J=7.4 Hz, 2H), 1.31 (t, J=7.1 Hz, 3H).

### Example 56: 2-{[2-carboxy-5-(4,4-dimethylpiperidin-1-yl)phenyl]carbamoyl}-5-hydroxybenzene-1,4-dicarboxylic acid (GO-0003746)

2-{[2-carboxy-5-(4,4-dimethylpiperidin-1-yl)phenyl]carbamoyl}-5-hydroxybenzene-1,4-dicarboxylic acid was prepared at the Step 53.1 of Example 53. After preparative HPLC the target compound was selected as the isomer, which matched the NMR spectra.

1H NMR (250 MHz, DMSO-d6) δ 12.15 (d, J=10.7 Hz, 1H), 8.21 (d, J=32.5 Hz, 2H), 7.82 (dd, J=9.1, 3.2 Hz, 1H), 7.18 (d, J=19.8 Hz, 1H), 6.72 (d, J=9.3 Hz, 1H), 3.37 (s, 5H), 1.43 (s, 4H), 0.99 (s, 6H).

### Example 57: 2-{[2-carboxy-5-(4,4-dimethylcyclohexyl)phenyl]carbamoyl}-5-hydroxybenzene-1,4-dicarboxylic acid (GO-0003747)

2-{[2-carboxy-5-(4,4-dimethylcyclohexyl)phenyl]carbamoyl}-5-hydroxybenzene-1,4-dicarboxylic acid was prepared at the Step 52.1 of Example 52. After preparative HPLC the target compound was selected as the isomer, which matched the NMR spectra.

### Example 58 4-[(5-{2-azabicyclo[2.2.1]heptan-2-yl}-2-carboxyphenyl)carbamoyl]-6-hydroxybenzene-1,3-dicarboxylic acid (GO-0003748)

4-[(5-{2-azabicyclo[2.2.1]heptan-2-yl}-2-carboxyphenyl)carbamoyl]-6-hydroxybenzene-1,3-dicarboxylic acid was prepared in one step.

### Step 58.1 Preparation of 4-((5-(2-azabicyclo[2.2.1]heptan-2-yl)-2-carboxyphenyl)carbamoyl)-6-hydroxyisophthalic acid and 2-((5-(2-azabicyclo[2.2.1]heptan-2-yl)-2-carboxyphenyl)carbamoyl)-5-hydroxyterephthalic acid

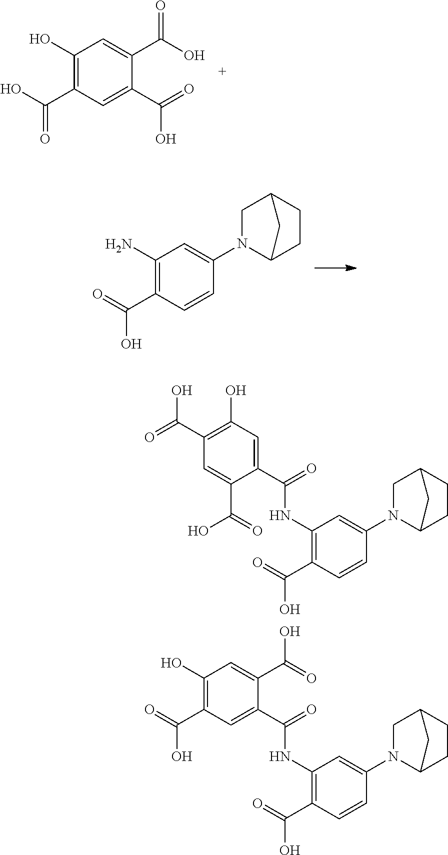

Using a procedure identical to that used above for Example 55, 5-hydroxybenzene-1,2,4-tricarboxylic acid (0.184 g) was reacted with 2-amino-4-(2-azabicyclo[2.2.1]heptan-2-yl)benzoic acid (0.094 g) to afford 4-((5-(2-azabicyclo[2.2.1]heptan-2-yl)-2-carboxyphenyl)carbamoyl)-6-hydroxyisophthalic acid and 2-((5-(2-azabicyclo[2.2.1]heptan-2-yl)-2-carboxyphenyl)carbamoyl)-5-hydroxyterephthalic acid after separation and purification by preparative HPLC. After preparative HPLC the target compound was selected as the isomer, which matched the NMR spectra.

1H NMR (250 MHz, DMSO-d6) δ 11.80 (s, 1H), 8.34 (s, 1H), 7.93-7.64 (m, 2H), 7.13 (s, 1H), 6.32 (d, J=9.3 Hz, 1H), 4.26 (s, 2H), 3.43 (d, J=7.3 Hz, 1H), 2.86 (d, J=8.9 Hz, 1H), 2.70-2.59 (m, 1H), 1.87-1.21 (m, 7H).

### Example 60: 2-{[2′,4′-dichloro-4-(ethoxycarbonyl)-[1,1′-biphenyl]-3-yl]carbamoyl}-5-hydroxybenzene-1,4-dicarboxylic acid (GO-0003751)

2-{[2′,4′-dichloro-4-(ethoxycarbonyl)-[1,1′-biphenyl]-3-yl]carbamoyl}-5-hydroxybenzene-1,4-dicarboxylic acid was prepared at the Step 55.1 of Example 55. After preparative HPLC the target compound was selected as the isomer, which didn't match the NMR spectra of undesirable isomer.

### Example 61: 2-[(5-{2-azabicyclo[2.2.1]heptan-2-yl}-2-carboxyphenyl)carbamoyl]-5-hydroxybenzene-1,4-dicarboxylic acid (GO-0003751)

2-[(5-{2-azabicyclo[2.2.1]heptan-2-yl}-2-carboxyphenyl)carbamoyl]-5-hydroxybenzene-1,4-dicarboxylic acid was prepared at the Step 58.1 of Example 58. After preparative HPLC the target compound was selected as the isomer, which matched the NMR spectra.

1H NMR (250 MHz, DMSO-d6) δ 12.25 (d, J=10.9 Hz, 1H), 8.15 (s, 1H), 7.95-7.70 (m, 2H), 7.17 (d, J=19.7 Hz, 1H), 6.32 (d, J=8.9 Hz, 1H), 4.25 (s, 1H), 3.43 (d, J=7.4 Hz, 1H), 2.86 (d, J=9.0 Hz, 1H), 2.64 (s, 1H), 1.84-1.28 (m, 7H).

### Example 62 4-[(5-{2-azabicyclo[2.2.1]heptan-2-yl}-2-carboxyphenyl)carbamoyl]-6-hydroxybenzene-1,3-dicarboxylic acid (GO-0003754)

2-((2-((2-(dimethylamino)ethoxy)carbonyl)-4-(1H-imidazol-5-yl)phenyl)carbamoyl)-5-hydroxyterephthalic acid was prepared in several steps.

### Step 62.1 Preparation of methyl 2-amino-5-(4,4,5,5-tetramethyl-1,3,2-dioxaborolan-2-yl)benzoate

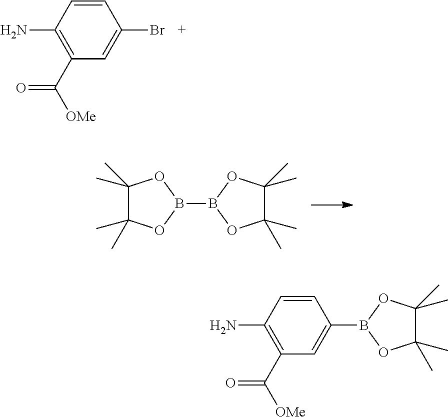

Methyl 2-amino-5-bromobenzoate (1.0 g, 4.34 mmol) and 4,4,4′,4′,5,5,5′,5′-octamethyl-2,2′-bi(1,3,2-dioxaborolane) (1.32 g, 5.20 mmol) were dissolved in dioxane (24 mL) and treated with PdCl₂dppf (0.176 g) and potassium acetate (1.28 g) at 85° C. for 15 h. The cooled reaction mixture was poured into brine and extracted (3×) with ethyl acetate. The combined organic layers were dried over sodium sulfate, filtered and concentrated to afford a residue which was purified by flash chromatography (hexane/ethyl acetate 0-25%). The purified product was identified as methyl 2-amino-5-(4,4,5,5-tetramethyl-1,3,2-dioxaborolan-2-yl)benzoate (1.15 g, 95%).

### Step 62.2 Preparation of methyl 2-amino-5-(1-trityl-1H-imidazol-5-yl)benzoate

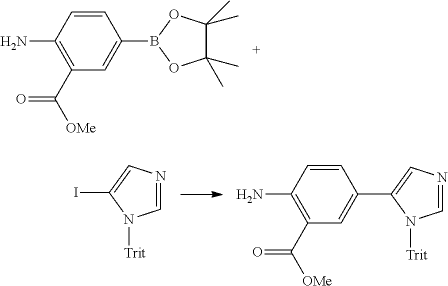

Methyl 2-amino-5-(4,4,5,5-tetramethyl-1,3,2-dioxaborolan-2-yl)benzoate (1.23 g, 4.438 mmol) and 5-iodo-1-trityl-1H-imidazole (1.29 g, 2.96 mmol) were dissolved in ethanol (5 mL) and toluene (10 mL) and treated with PdCl₂dppf (0.217 g, 0.296 mmol) and sodium carbonate (2N, 5.92 mL) for 120° C. for 100 min in a microwave reactor. The cooled reaction mixture was poured into brine and extracted (3×) with ethyl acetate. The combined organic layers were dried over sodium sulfate, filtered and concentrated to afford a residue which was purified by flash chromatography (hexane/ethyl acetate 0-25%) to afford pure methyl 2-amino-5-(1-trityl-1H-imidazol-5-yl)benzoate (0.935 g, 68.8% yield).

### Step 62.3 Preparation of methyl 2-amino-5-(1H-imidazol-5-yl)benzoate

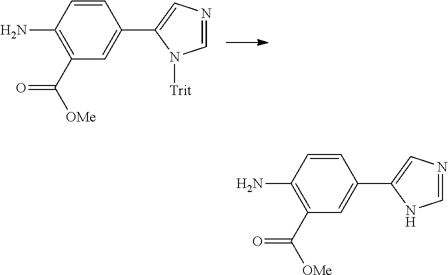

Methyl 2-amino-5-(1-trityl-1H-imidazol-5-yl)benzoate (1.34 g, 2.638 mmol) was dissolved in dichloromethane (40 mL) and treated with TFA (10 mL) at room temperature for 2 h. The mixture was diluted with dichloromethane (40 mL) and evaporated to dryness. Chromatography (dichloromethane/methanol (0-20%) afforded pure methyl 2-amino-5-(1H-imidazol-5-yl)benzoate (0.505 g, 86% yield).

### Step 62.4 Preparation of 2-amino-5-(1H-imidazol-5-yl)benzoic acid

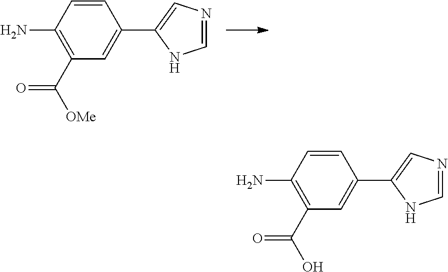 [SMILES: COC(=O)c1cc(-c2cnc[nH]2)ccc1N ; Nc1ccc(-c2cnc[nH]2)cc1C(=O)O]

Methyl 2-amino-5-(1H-imidazol-5-yl)benzoate was hydrolyzed by treatment with sodium hydroxide (4.64 mL, 2M) in methanol (20 mL) by stirring overnight at room temperature followed by heating at 50° C. for 2 h. Acidification (0.1M HCl) and evaporation by lyophilization gave a yellow powder which was washed with methanol, the filtrate collected, evaporated and dried to afford pure 2-amino-5-(1H-imidazol-5-yl)benzoic acid (0.406 g, 86% yield).

### Step 62.5 Preparation of 2-(dimethylamino)ethyl 2-amino-5-(1H-imidazol-5-yl)benzoate

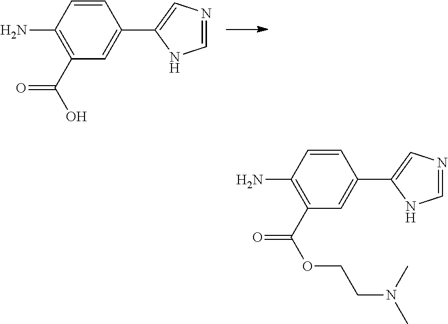 [SMILES: Nc1ccc(-c2cnc[nH]2)cc1C(=O)O ; CN(C)CCOC(=O)c1cc(-c2cnc[nH]2)ccc1N]

2-Amino-5-(1H-imidazol-5-yl)benzoic acid (0.203 g, 1 mmol) was esterified with 2-(dimethylamino)ethan-1-ol (1 mL) by treatment with HATU (0.570 g, 1.5 mmol), diisopropylethylamine (0.523 mL) in DMF (10 mL) at room temperature overnight. The reaction mixture was poured into water and extracted with 2-methyl tetrahydrofuran (3×). The combined organic layers were dried over sodium sulfate, filtered, concentrated and purified by flash chromatography (dichloromethane/methane 0-25%) to afford pure 2-(dimethylamino)ethyl 2-amino-5-(1H-imidazol-5-yl)benzoate (0.135 g, 49% yield).

### Step 62.5 Preparation of 2-((2-((2-(dimethylamino)ethoxy)carbonyl)-4-(1H-imidazol-5-yl)phenyl)carbamoyl)-5-hydroxyterephthalic acid and 4-((2-((2-(dimethylamino)ethoxy)carbonyl)-4-(1H-imidazol-5-yl)phenyl)carbamoyl)-6-hydroxyisophthalic acid

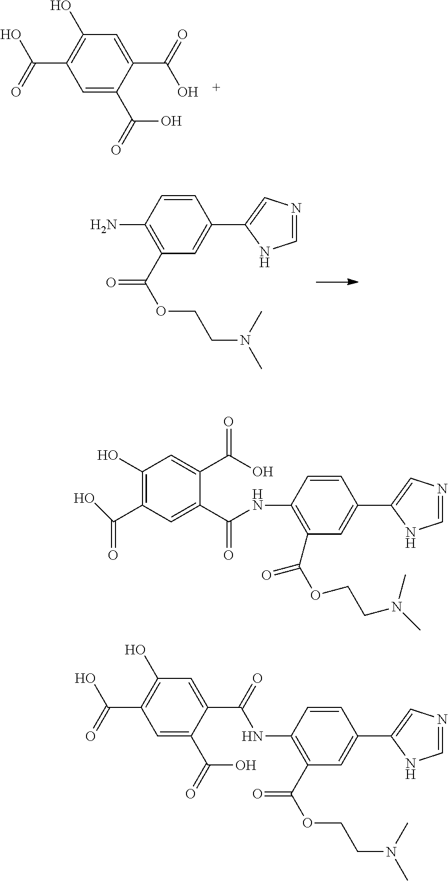 [SMILES: O=C(O)c1cc(C(=O)O)c(C(=O)O)cc1O ; CN(C)CCOC(=O)c1cc(-c2cnc[nH]2)ccc1N ; CN(C)CCOC(=O)c1cc(-c2cnc[nH]2)ccc1NC(=O)c1cc(C(=O)O)c(O)cc1C(=O)O ; CN(C)CCOC(=O)c1cc(-c2cnc[nH]2)ccc1NC(=O)c1cc(O)c(C(=O)O)cc1C(=O)O]

Using the same procedure as was used for Example 78, 5-hydroxybenzene-1,2,4-tricarboxylic acid (0.222 mg, 0.98 mmol) and 2-(dimethylamino)ethyl 2-amino-5-(1H-imidazol-5-yl)benzoate 0.135 g, 0.492 mmol) were reacted to afford 2-((2-((2-(dimethylamino)ethoxy)carbonyl)-4-(1H-imidazol-5-yl)phenyl)carbamoyl)-5-hydroxyterephthalic acid and 4-((2-((2-(dimethylamino)ethoxy)carbonyl)-4-(1H-imidazol-5-yl)phenyl)carbamoyl)-6-hydroxyisophthalic acid after purification by preparative HPLC. After preparative HPLC the target compound was selected as the isomer, which matched the NMR spectra.

1H NMR (250 MHz, DMSO-d6) δ 10.87 (s, 1H), 9.10 (s, 1H), 8.51-8.27 (m, 3H), 8.26-7.97 (m, 2H), 7.09 (d, J=9.0 Hz, 1H), 4.60 (s, 3H), 3.52 (s, 3H), 2.95-2.73 (m, 6H).

### Example 65 4-{[2′,4′-dichloro-4-({[1-(dimethylamino)propan-2-yl]oxy}carbonyl)-[1,1′-biphenyl]-3-yl]carbamoyl}-6-hydroxybenzene-1,3-dicarboxylic acid (GO-0003757)

4-{[2′,4′-dichloro-4-({[1-(dimethylamino)propan-2-yl]oxy}carbonyl)-[1,1′-biphenyl]-3-yl]carbamoyl}-6-hydroxybenzene-1,3-dicarboxylic acid was prepared in one step.

### Step 65.1 Preparation of 4-((2′,4′-dichloro-4-(((1-(dimethylamino)propan-2-yl)oxy)carbonyl)-[1,1′-biphenyl]-3-yl)carbamoyl)-6-hydroxyisophthalic acid and 2-((2′,4′-dichloro-4-(((1-(dimethylamino)propan-2-yl)oxy)carbonyl)-[1,1′-biphenyl]-3-yl)carbamoyl)-5-hydroxyterephthalic acid

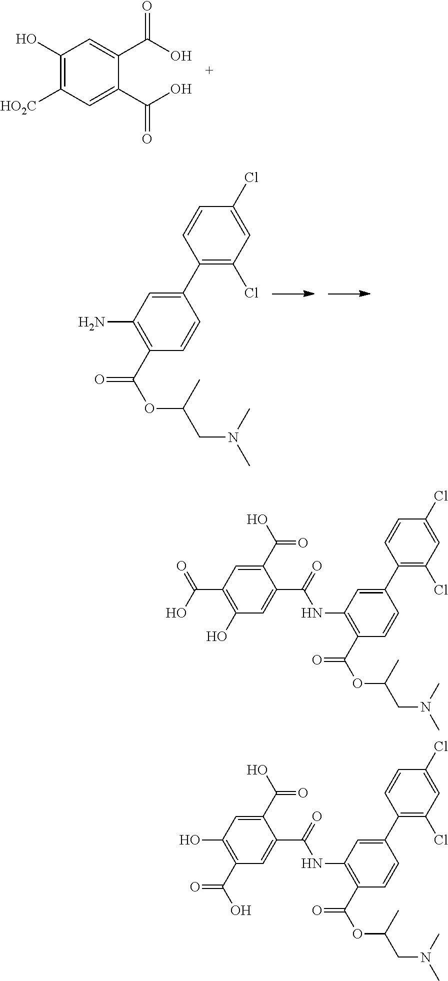

Using a procedure identical to that used for Example 55, 5-hydroxybenzene-1,2,4-tricarboxylic acid (0.080 g) was reacted with 1-(dimethylamino)propan-2-yl 3-amino-2′,4′-dichloro-[1,1′-biphenyl]-4-carboxylate (0.065 g) to afford 4-((2′,4′-dichloro-4-(((1-(dimethylamino)propan-2-yl)oxy)carbonyl)-[1,1′-biphenyl]-3-yl)carbamoyl)-6-hydroxyisophthalic acid and 2-((2′,4′-dichloro-4-(((1-(dimethylamino)propan-2-yl)oxy)carbonyl)-[1,1′-biphenyl]-3-yl)carbamoyl)-5-hydroxyterephthalic acid (NSQP00698) after separation and purification by preparative HPLC. After preparative HPLC the target compound was selected as the isomer, which matched the NMR spectra.

1H NMR (250 MHz, DMSO-d6) δ 10.92 (s, 1H), 9.52 (s, 1H), 8.35 (s, 2H), 8.14 (d, J=8.2 Hz, 1H), 7.83 (d, J=2.1 Hz, 1H), 7.61 (dd, J=8.3, 2.1 Hz, 1H), 7.48 (d, J=8.3 Hz, 1H), 7.35 (dd, J=8.2, 1.8 Hz, 1H), 7.07 (s, 1H), 5.44 (s, 1H), 3.63-3.28 (m, 4H), 2.85 (s, 6H), 1.34 (d, J=6.3 Hz, 3H).

### Example 66 4-[(2′,4′-dichloro-4-{[2-(dimethylamino)propoxy]carbonyl}-[1,1′-biphenyl]-3-yl)carbamoyl]-6-hydroxybenzene-1,3-dicarboxylic acid (GO-0003757)

4-[(2′,4′-dichloro-4-{[2-(dimethylamino)propoxy]carbonyl}-[1,1′-biphenyl]-3-yl)carbamoyl]-6-hydroxybenzene-1,3-dicarboxylic acid was prepared in one step. Step 66.1 Preparation of 4-((2′,4′-dichloro-4-((2-(dimethylamino)propoxy)carbonyl)-[1,1′-biphenyl]-3-yl)carbamoyl)-6-hydroxyisophthalic acid and 2-((2′,4′-dichloro-4-((2-(dimethylamino)propoxy)carbonyl)-[1,1′-biphenyl]-3-yl)carbamoyl)-5-hydroxyterephthalic acid

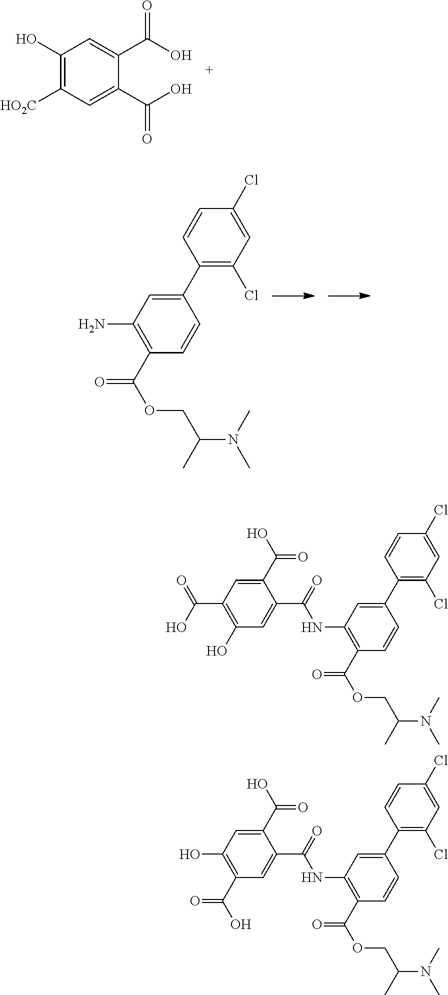

Using a procedure identical to that used for Example 55, 5-hydroxybenzene-1,2,4-tricarboxylic acid (0.080 g) was reacted with 2-(dimethylamino)propyl 3-amino-2′,4′-dichloro-[1,1′-biphenyl]-4-carboxylate (0.065 g) to afford 4-((2′,4′-dichloro-4-((2-(dimethylamino)propoxy)carbonyl)-[1,1′-biphenyl]-3-yl)carbamoyl)-6-hydroxyisophthalic acid and 2-((2′,4′-dichloro-4-((2-(dimethylamino)propoxy)carbonyl)-[1,1′-biphenyl]-3-yl)carbamoyl)-5-hydroxyterephthalic acid after separation and purification by preparative HPLC. After preparative HPLC the target compound was selected as the isomer, which didn't match the NMR spectra of undesirable isomer.

### Example 67 4-[(2′,4′-dichloro-4-{[(1-methylpyrrolidin-3-yl)oxy]carbonyl}-[1,1′-biphenyl]-3-yl)carbamoyl]-6-hydroxybenzene-1,3-dicarboxylic acid (GO-0003759)

4-[(2′,4′-dichloro-4-{[(1-methylpyrrolidin-3-yl)oxy]carbonyl}-[1,1′-biphenyl]-3-yl)carbamoyl]-6-hydroxybenzene-1,3-dicarboxylic acid was prepared in one step. Step 67.1 Preparation of 4-((2′,4′-dichloro-4-(((1-methylpyrrolidin-3-yl)oxy)carbonyl)-[1,1′-biphenyl]-3-yl)carbamoyl)-6-hydroxyisophthalic acid and 2-((2′,4′-dichloro-4-(((1-methylpyrrolidin-3-yl)oxy)carbonyl)-[1,1′-biphenyl]-3-yl)carbamoyl)-5-hydroxyterephthalic acid

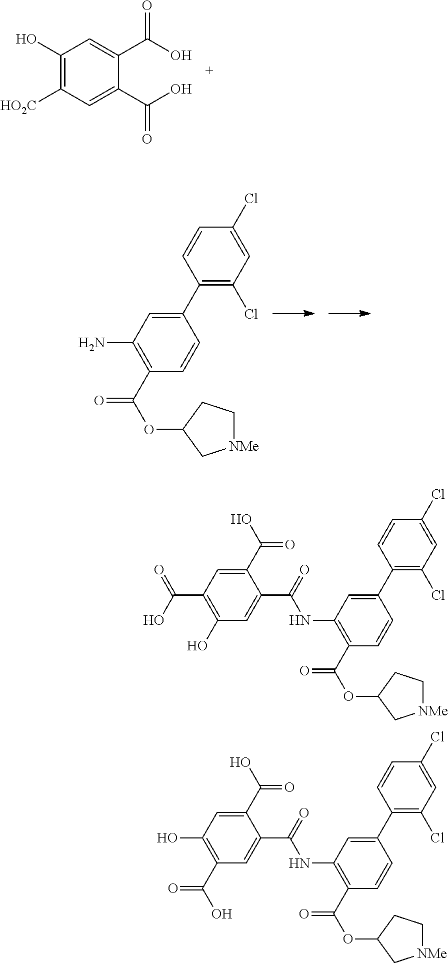

Using a procedure identical to that used for Example 55, 5-hydroxybenzene-1,2,4-tricarboxylic acid (0.075 g) was reacted with 1-methylpyrrolidin-3-yl 3-amino-2′,4′-dichloro-[1,1′-biphenyl]-4-carboxylate (0.060 g) to afford 4-((2′,4′-dichloro-4-(((1-methylpyrrolidin-3-yl)oxy)carbonyl)-[1,1′-biphenyl]-3-yl)carbamoyl)-6-hydroxyisophthalic acid and 2-((2′,4′-dichloro-4-(((1-methylpyrrolidin-3-yl)oxy)carbonyl)-[1,1′-biphenyl]-3-yl)carbamoyl)-5-hydroxyterephthalic acid after separation and purification by preparative HPLC. After preparative HPLC the target compound was selected as the isomer, which matched the NMR spectra. 1H NMR (250 MHz, DMSo-d6) δ 10.81 (s, 1H), 8.36 (d, J=3.3 Hz, 1H), 8.29 (s, 1H), 8.12 (d, J=7.6 Hz, 1H), 7.82 (d, J=2.1 Hz, 1H), 7.60 (dd, J=8.3, 2.1 Hz, 1H), 7.49 (d, J=8.4 Hz, 1H), 7.35 (dd, J=8.2, 1.8 Hz, 1H), 7.07 (d, J=11.2 Hz, 1H), 5.54 (s, 1H), 2.90 (s, 6H), 2.22 (s, 3H).

### Example 69 4-[(2′,4′-dichloro-4-{[(1-methylazetidin-3-yl)oxy]carbonyl}-[1,1′-biphenyl]-3-yl)carbamoyl]-6-hydroxybenzene-1,3-dicarboxylic acid (GO-0003762)

4-[(2′,4′-dichloro-4-{[(1-methylazetidin-3-yl)oxy]carbonyl}-[1,1′-biphenyl]-3-yl)carbamoyl]-6-hydroxybenzene-1,3-dicarboxylic acid was prepared in one step. Step 69.1 Preparation of 4-((2′,4′-dichloro-4-(((1-methylazetidin-3-yl)oxy)carbonyl)-[1,1′-biphenyl]-3-yl)carbamoyl)-6-hydroxyisophthalic acid and 2-((2′,4′-dichloro-4-(((1-methylazetidin-3-yl)oxy)carbonyl)-[1,1′-biphenyl]-3-yl)carbamoyl)-5-hydroxyterephthalic acid

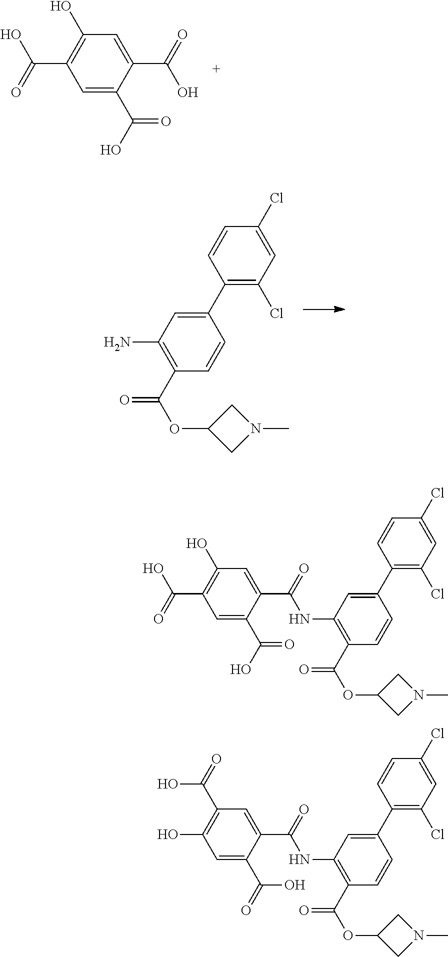

Using a procedure identical to that used for Example 55, 5-hydroxybenzene-1,2,4-tricarboxylic acid (0.084 g) was reacted with 1-methylazetidin-3-yl 3-amino-2′,4′-dichloro-[1,1′-biphenyl]-4-carboxylate (0.065 g) to afford 4-((2′,4′-dichloro-4-(((1-methylazetidin-3-yl)oxy)carbonyl)-[1,1′-biphenyl]-3-yl)carbamoyl)-6-hydroxyisophthalic acid (NSQP00706) and 2-((2′,4′-dichloro-4-(((1-methylazetidin-3-yl)oxy)carbonyl)-[1,1′-biphenyl]-3-yl)carbamoyl)-5-hydroxyterephthalic acid (NSQP00707) after separation and purification by preparative HPLC. After preparative HPLC the target compound was selected as the isomer, which matched the NMR spectra. 1H NMR (250 MHz, DMSO-d6) δ 10.74 (s, 1H), 8.36 (d, J=4.3 Hz, 1H), 8.19 (s, 1H), 8.10 (dd, J=8.2, 2.7 Hz, 1H), 7.83 (d, J=2.1 Hz, 1H), 7.66-7.57 (m, 1H), 7.50 (d, J=8.3 Hz, 1H), 7.39 (d, J=8.3 Hz, 1H), 7.03 (d, J=14.9 Hz, 1H), 5.39 (d, J=30.8 Hz, 1H), 4.74-4.14 (m, 5H), 2.90 (d, J=4.4 Hz, 3H).

### Example 70 4-[(2′,4′-dichloro-4-{[(dimethylcarbamoyl)methoxy]carbonyl}-[1,1′-biphenyl]-3-yl)carbamoyl]-6-hydroxybenzene-1,3-dicarboxylic acid (GO-0003763)

4-[(2′,4′-dichloro-4-{[(dimethylcarbamoyl)methoxy]carbonyl}-[1,1′-biphenyl]-3-yl)carbamoyl]-6-hydroxybenzene-1,3-dicarboxylic acid was prepared in one step. Step 70.1 Preparation of 4-((2′,4′-dichloro-4-((2-(dimethylamino)-2-oxoethoxy)carbonyl)-[1,1′-biphenyl]-3-yl)carbamoyl)-6-hydroxyisophthalic acid and 2-((2′,4′-dichloro-4-((2-(dimethylamino)-2-oxoethoxy)carbonyl)-[1,1′-biphenyl]-3-yl)carbamoyl)-5-hydroxy-terephthalic acid

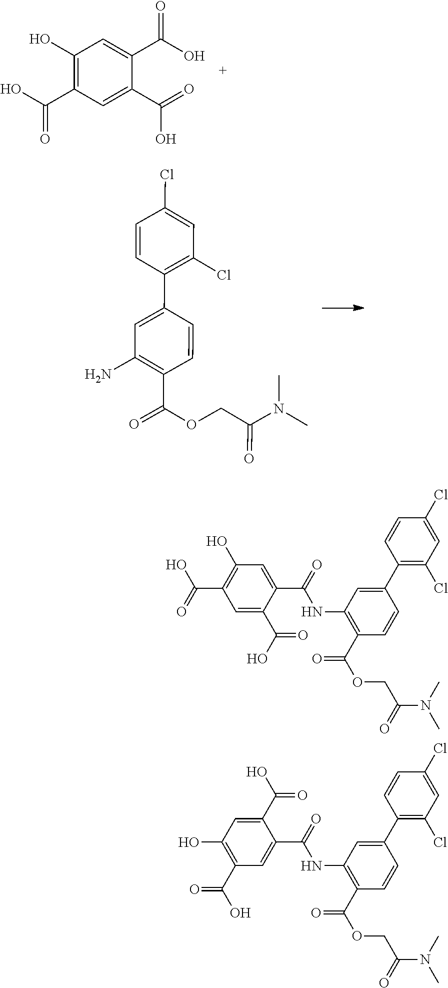

Using a procedure identical to that used for Example 55, 5-hydroxybenzene-1,2,4-tricarboxylic acid (0.074 g) was reacted with 2-(dimethylamino)-2-oxoethyl 3-amino-2′,4′-dichloro-[1,1′-biphenyl]-4-carboxylate (0.060 g) to afford 4-((2′,4′-dichloro-4-((2-(dimethylamino)-2-oxoethoxy)carbonyl)-[1,1′-biphenyl]-3-yl)carbamoyl)-6-hydroxyisophthalic acid and 2-((2′,4′-dichloro-4-((2-(dimethylamino)-2-oxoethoxy)carbonyl)-[1,1′-biphenyl]-3-yl)carbamoyl)-5-hydroxyterephthalic acid after separation and purification by preparative HPLC. After preparative HPLC the target compound was selected as the isomer, which matched the NMR spectra.

1H NMR (250 MHz, DMSO-d6) δ 10.94 (s, 1H), 8.51 (s, 1H), 8.35 (s, 1H), 8.05 (d, J=8.2 Hz, 1H), 7.83 (s, 1H), 7.67-7.47 (m, 2H), 7.36 (d, J=7.4 Hz, 1H), 7.13 (s, 1H), 5.09 (s, 2H), 2.95 (s, 3H), 2.77 (s, 3H).

### Example 71 4-({4-[(2-aminoethoxy)carbonyl]-2′,4′-dichloro-[1,1′-biphenyl]-3-yl}carbamoyl)-6-hydroxybenzene-1,3-dicarboxylic acid (GO-0003764)

4-({4-[(2-aminoethoxy)carbonyl]-2′,4′-dichloro-[1,1′-biphenyl]-3-yl}carbamoyl)-6-hydroxybenzene-1,3-dicarboxylic acid was prepared in one step.

### Step 71.1 Preparation of 2-((4-((2-aminoethoxy)carbonyl)-2′,4′-dichloro-[1,1′-biphenyl]-3-yl)carbamoyl)-5-hydroxyterephthalic acid and 4-((4-((2-aminoethoxy)carbonyl)-2′,4′-dichloro-[1,1′-biphenyl]-3-yl)carbamoyl)-6-hydroxyisophthalic acid

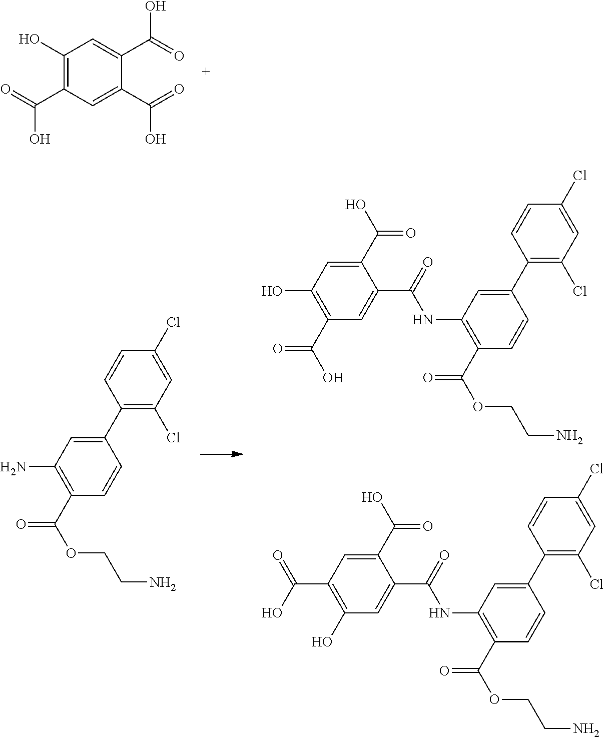 [SMILES: O=C(O)c1cc(C(=O)O)c(C(=O)O)cc1O ; NCCOC(=O)c1ccc(-c2ccc(Cl)cc2Cl)cc1N ; NCCOC(=O)c1ccc(-c2ccc(Cl)cc2Cl)cc1NC(=O)c1cc(C(=O)O)c(O)cc1C(=O)O ; NCCOC(=O)c1ccc(-c2ccc(Cl)cc2Cl)cc1NC(=O)c1cc(O)c(C(=O)O)cc1C(=O)O]

Using a procedure identical to that used for Example 55, 5-hydroxybenzene-1,2,4-tricarboxylic acid (0.070 g) was reacted with 2-((tert-butoxycarbonyl)amino)ethyl 3-amino-2′,4′-dichloro-[1,1′-biphenyl]-4-carboxylate (0.065 g) to afford a mixture of the Boc-protected analogs of the title compounds, which were deprotected by treatment with TFA (2 mL) to afford 2-((4-((2-aminoethoxy)carbonyl)-2′,4′-dichloro-[1,1′-biphenyl]-3-yl)carbamoyl)-5-hydroxyterephthalic acid and 4-((4-((2-aminoethoxy)carbonyl)-2′,4′-dichloro-[1,1′-biphenyl]-3-yl)carbamoyl)-6-hydroxyisophthalic acid after separation and purification by preparative HPLC. After preparative HPLC the target compound was selected as the isomer, which matched the NMR spectra.

1H NMR (250 MHz, DMSO-d6) δ 10.93 (d, J=9.1 Hz, 1H), 8.43-8.32 (m, 2H), 8.23 (dd, J=8.3, 4.8 Hz, 1H), 8.00 (s, 2H), 7.83 (d, J=1.7 Hz, 1H), 7.61 (dd, J=8.3, 2.1 Hz, 1H), 7.50 (d, J=8.3 Hz, 1H), 7.36 (d, J=8.7 Hz, 1H), 7.08 (d, J=14.2 Hz, 1H), 4.46 (s, 2H), 3.24 (s, 2H).

### Example 72 5-{[dimethyl(oxo)-λ⁶-sulfanylidene]carbamoyl}-2-{[4-(4,4-dimethylpiperidin-1-yl)pyridin-2-yl]carbamoyl}benzoic acid (GO-0003766)

5-{[dimethyl(oxo)-λ⁶-sulfanylidene]carbamoyl}-2-{[4-(4,4-dimethylpiperidin-1-yl)pyridin-2-yl]carbamoyl}benzoic acid was prepared in several steps.

### Step 72.1 Preparation of 4-(4,4-dimethylpiperidin-1-yl)pyridin-2-amine

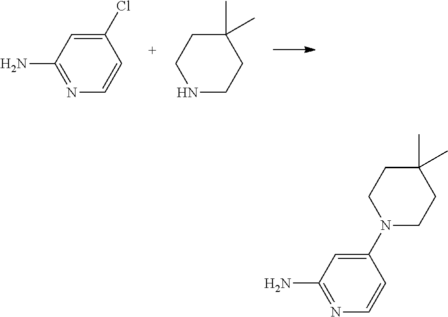

4-Chloropyridin-2-amine (0.200 g, 1.55 mmol) was dissolved in NMP (6 mL) and treated with 4,4-dimethylpiperidine (0.352 g, 3.11 mmol). The mixture was heated in a microwave reactor at 200° C. for 45 min. The cooled solution was poured into water, extracted (3×) with ethyl acetate, and the combined organic layers were washed with brine (2×), dried over sodium sulfate, filtered, concentrated and dried under vacuum to afford pure 4-(4,4-dimethylpiperidin-1-yl)pyridin-2-amine (0.295 g, 92% yield).

### Step 72.2 Preparation of 2-(4-(4,4-dimethylpiperidin-1-yl)pyridin-2-yl)-1,3-dioxoisoindoline-5-carboxylic acid

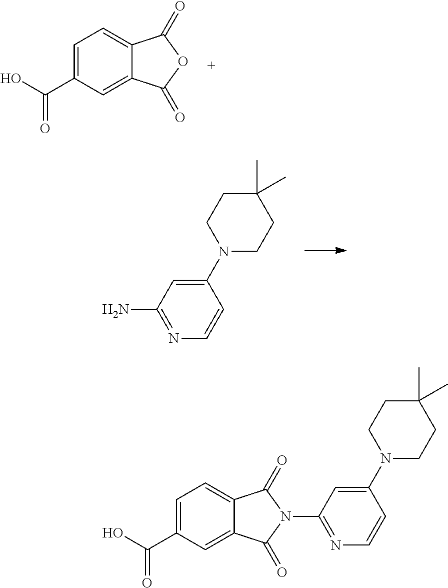 [SMILES: O=C(O)c1ccc2c(c1)C(=O)OC2=O ; CC1(C)CCN(c2ccnc(N)c2)CC1 ; CC1(C)CCN(c2ccnc(N3C(=O)c4ccc(C(=O)O)cc4C3=O)c2)CC1]

Using General Procedure #9, 4-(4,4-dimethylpiperidin-1-yl)pyridin-2-amine(0.103 g, 0.5 mmol) and 1,3-dioxo-1,3-dihydroisobenzofuran-5-carboxylic acid (0.096 g, 0.5 mmol) were reacted in isobutyric acid at 175° C. in a microwave reactor for 6 h to afford 2-(4-(4,4-dimethylpiperidin-1-yl)pyridin-2-yl)-1,3-dioxoisoindoline-5-carboxylic acid (0.121 g, 64%) after flash chromatography (DCM/MeOH 0-20%).

### Step 72.3 Preparation of N-(dimethyl(oxo)-λ6-sulfaneylidene)-2-(4-(4,4-dimethylpiperidin-1-yl)pyridin-2-yl)-1,3-dioxoisoindoline-5-carboxamide

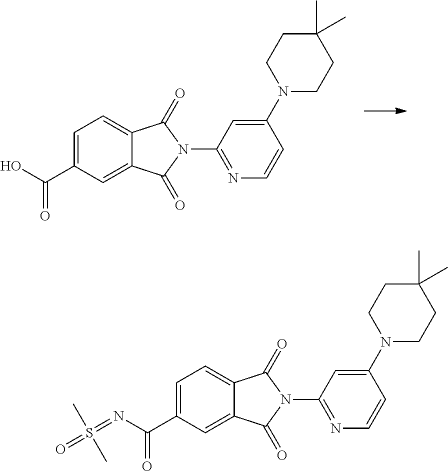 [SMILES: CC1(C)CCN(c2ccnc(N3C(=O)c4ccc(C(=O)O)cc4C3=O)c2)CC1 ; CC1(C)CCN(c2ccnc(N3C(=O)c4ccc(C(=O)N=S(C)(C)=O)cc4C3=O)c2)CC1]

Using General Procedure #11, 2-(4-(4,4-dimethylpiperidin-1-yl)pyridin-2-yl)-1,3-dioxoisoindoline-5-carboxylic acid (0.121 g, 0.32 mmol) was converted to N-(dimethyl(oxo)-λ6-sulfaneylidene)-2-(4-(4,4-dimethylpiperidin-1-yl)pyridin-2-yl)-1,3-dioxoisoindoline-5-carboxamide (0.1075 g, 74% yield) after flash chromatography (hexane/ethyl acetate 0-100%).

### Step 72.4 Preparation of 5-((dimethyl(oxo)-λ6-sulfaneylidene)carbamoyl)-2-((4-(4,4-dimethylpiperidin-1-yl)pyridin-2-yl)carbamoyl)benzoic acid and 4-((dimethyl(oxo)-λ6-sulfaneylidene)carbamoyl)-2-((4-(4,4-dimethylpiperidin-1-yl)pyridin-2-yl)carbamoyl)benzoic acid

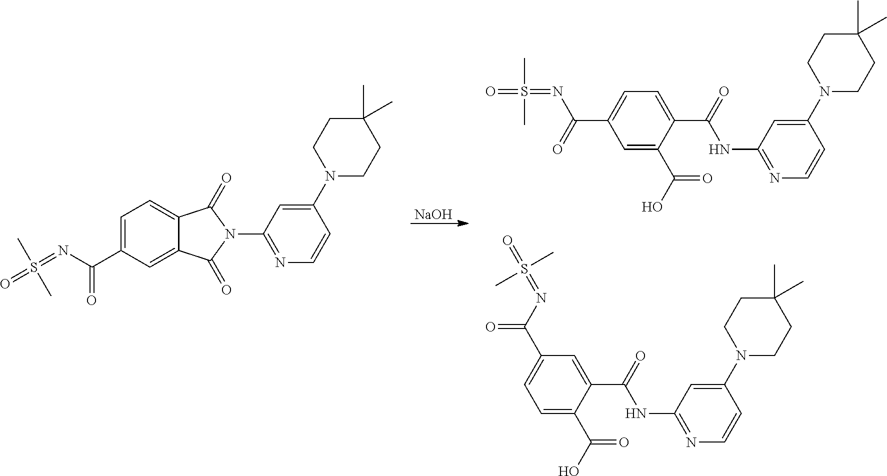 [SMILES: CC1(C)CCN(c2ccnc(N3C(=O)c4ccc(C(=O)N=S(C)(C)=O)cc4C3=O)c2)CC1 ; "NaOH" ; CC1(C)CCN(c2ccnc(NC(=O)c3cc(C(=O)N=S(C)(C)=O)ccc3C(=O)O)c2)CC1 ; CC1(C)CCN(c2ccnc(NC(=O)c3ccc(C(=O)N=S(C)(C)=O)cc3C(=O)O)c2)CC1]

Using General Procedure #7, N-(dimethyl(oxo)-λ6-sulfaneylidene)-2-(4-(4,4-dimethylpiperidin-1-yl)pyridin-2-yl)-1,3-dioxoisoindoline-5-carboxamide (0.1075 g, 0.24 mmol) was ring-opened with sodium hydroxide to afford 5-((dimethyl(oxo)-λ6-sulfaneylidene)carbamoyl)-2-((4-(4,4-dimethylpiperidin-1-yl)pyridin-2-yl)carbamoyl)benzoic acid and 4-((dimethyl(oxo)-λ6-sulfaneylidene)carbamoyl)-2-((4-(4,4-dimethylpiperidin-1-yl)pyridin-2-yl)carbamoyl)benzoic acid after HPLC purification. After preparative HPLC the target compound was selected as the isomer, which matched the NMR spectra.

1H NMR (250 MHz, DMSO-d6) δ 11.88 (s, 1H), 8.29-8.14 (m, 2H), 8.06 (d, J=8.0 Hz, 1H), 7.94 (d, J=7.5 Hz, 1H), 7.07 (d, J=7.3 Hz, 1H), 6.68 (s, 1H), 3.57 (s, 4H), 1.44 (s, 4H), 1.00 (s, 6H).

### Example 73: 2-{[2′,4′-dichloro-4-({[1-(dimethylamino)propan-2-yl]oxy}carbonyl)-[1,1′-biphenyl]-3-yl]carbamoyl}-5-hydroxybenzene-1,4-dicarboxylic acid (GO-0003768)

2-{[2′,4′-dichloro-4-({[1-(dimethylamino)propan-2-yl]oxy}carbonyl)-[1,1′-biphenyl]-3-yl]carbamoyl}-5-hydroxybenzene-1,4-dicarboxylic acid was prepared at the Step 65.1 of Example 65. After preparative HPLC the target compound was selected as the isomer, which matched the NMR spectra.

1H NMR (250 MHz, DMSO-d6) δ 11.22 (s, 1H), 9.53 (s, 1H), 8.37 (s, 1H), 8.22-8.09 (m, 2H), 7.83 (s, 1H), 7.62 (s, 1H), 7.51 (d, J=8.0 Hz, 1H), 7.36 (d, J=8.2 Hz, 1H), 7.17 (d, J=13.4 Hz, 1H), 5.52 (s, 1H), 3.69-3.31 (m, 3H), 2.89 (s, 6H), 1.37 (d, J=6.2 Hz, 3H).

### Example 74: 2-[(2′,4′-dichloro-4-{[2-(dimethylamino)propoxy]carbonyl}-[1,1′-biphenyl]-3-yl)carbamoyl]-5-hydroxybenzene-1,4-dicarboxylic acid (GO-0003769)

2-[(2′,4′-dichloro-4-{[2-(dimethylamino)propoxy]carbonyl}-[1,1′-biphenyl]-3-yl)carbamoyl]-5-hydroxybenzene-1,4-dicarboxylic acid was prepared at the Step 66.1 of Example 66. After preparative HPLC the target compound was selected as the isomer, which matched the NMR spectra.

### Example 75: 2-[(2′,4′-dichloro-4-{[(1-methylpyrrolidin-3-yl)oxy]carbonyl}-[1,1′-biphenyl]-3-yl)carbamoyl]-5-hydroxybenzene-1,4-dicarboxylic acid (GO-0003770)

2-[(2′,4′-dichloro-4-{[(1-methylpyrrolidin-3-yl)oxy]carbonyl}-[1,1′-biphenyl]-3-yl)carbamoyl]-5-hydroxybenzene-1,4-dicarboxylic acid was prepared at the Step 67.1 of Example 67. After preparative HPLC the target compound was selected as the isomer, which didn't match the NMR spectra of undesirable isomer.

### Example 77: 2-[(2′,4′-dichloro-4-{[(1-methylazetidin-3-yl)oxy]carbonyl}-[1,1′-biphenyl]-3-yl)carbamoyl]-5-hydroxybenzene-1,4-dicarboxylic acid (GO-0003773)

2-[(2′,4′-dichloro-4-{[(1-methylazetidin-3-yl)oxy]carbonyl}-[1,1′-biphenyl]-3-yl)carbamoyl]-5-hydroxybenzene-1,4-dicarboxylic acid was prepared at the Step 69.1 of Example 69. After preparative HPLC the target compound was selected as the isomer, which didn't match the NMR spectra of undesirable isomer.

### Example 78 2-({2′,4′-dichloro-4-[4-(dimethylamino)butanoyl]-[1,1′-biphenyl]-3-yl}carbamoyl)-5-hydroxybenzene-1,4-dicarboxylic acid (GO-0003774)

2-({2′,4′-dichloro-4-[4-(dimethylamino)butanoyl]-[1,1′-biphenyl]-3-yl}carbamoyl)-5-hydroxybenzene-1,4-dicarboxylic acid was prepared in one step.

### Step 78.1 Preparation of 4-((2′,4′-dichloro-4-(4-(dimethylamino)butanoyl)-[1,1′-biphenyl]-3-yl)-carbamoyl)-6-hydroxyisophthalic acid and 2-((2′,4′-dichloro-4-(4-(dimethylamino)butanoyl)-[1,1′-biphenyl]-3-yl)carbamoyl)-5-hydroxyterephthalic acid

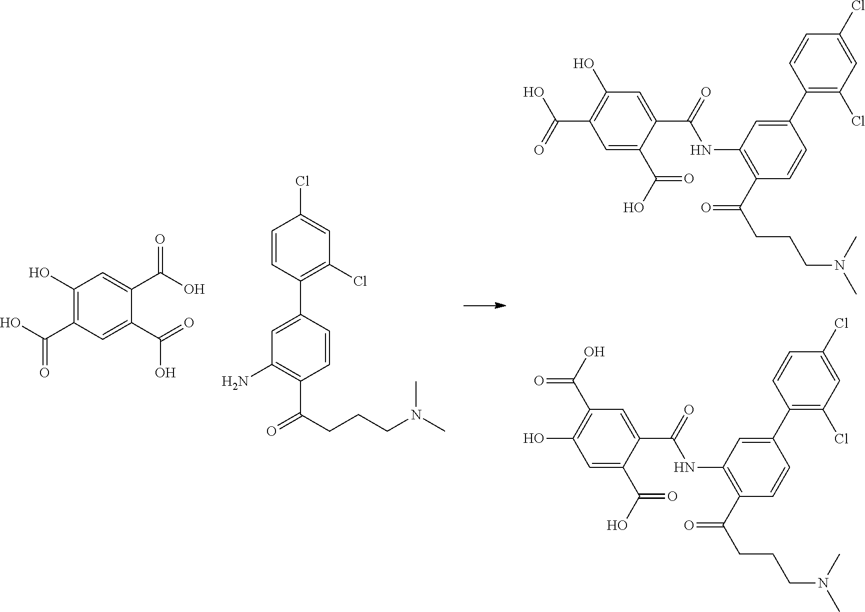

Using a procedure identical to that used for Example 55, 5-hydroxybenzene-1,2,4-tricarboxylic acid (0.084 g) was reacted with 1-(3-amino-2′,4′-dichloro-[1,1′-biphenyl]-4-yl)-4-(dimethylamino)butan-1-one (0.065 g) to afford 4-((2′,4′-dichloro-4-(4-(dimethylamino)butanoyl)-[1,1′-biphenyl]-3-yl)carbamoyl)-6-hydroxyisophthalic acid (NSQP00701) and 2-((2′,4′-dichloro-4-(4-(dimethylamino)butanoyl)-[1,1′-biphenyl]-3-yl)carbamoyl)-5-hydroxyterephthalic acid after separation and purification by preparative HPLC. After preparative HPLC the target compound was selected as the isomer, which matched the NMR spectra.

1H NMR (250 MHz, DMSO-d6) δ 11.68 (d, J=14.5 Hz, 1H), 9.37 (s, 1H), 8.31 (dd, J=4.9, 1.7 Hz, 1H), 8.17-8.02 (m, 2H), 7.82 (d, J=2.0 Hz, 1H), 7.66-7.44 (m, 2H), 7.43-7.29 (m, 1H), 7.16 (d, J=16.1 Hz, 1H), 3.26 (s, 2H), 3.12 (s, 2H), 2.81 (d, J=4.0 Hz, 6H), 1.99 (s, 2H).

### Example 79: 2-[(2′,4′-dichloro-4-{[(dimethylcarbamoyl)methoxy]carbonyl}-[1,1′-biphenyl]-3-yl)carbamoyl]-5-hydroxybenzene-1,4-dicarboxylic acid (GO-0003775)

2-[(2′,4′-dichloro-4-{[(dimethylcarbamoyl)methoxy]carbonyl}-[1,1′-biphenyl]-3-yl)carbamoyl]-5-hydroxybenzene-1,4-dicarboxylic acid was prepared at the Step 70.1 of Example 70. After preparative HPLC the target compound was selected as the isomer, which didn't match the NMR spectra of undesirable isomer.

1H NMR (250 MHz, DMSO-d6) δ 11.08 (d, J=15.8 Hz, 1H), 8.51 (s, 1H), 8.13-7.97 (m, 2H), 7.82 (s, 1H), 7.66-7.47 (m, 2H), 7.40-7.28 (m, 1H), 7.20 (d, J=17.4 Hz, 1H), 5.15 (d, J=13.7 Hz, 2H), 2.97 (dd, J=2.5, 1.1 Hz, 3H), 2.80 (d, J=4.1 Hz, 3H).

### Example 80: 4-({2′,4′-dichloro-4-[4-(dimethylamino)butanoyl]-[1,1′-biphenyl]-3-yl}carbamoyl)-6-hydroxybenzene-1,3-dicarboxylic acid (GO-0003778)

4-({2′,4′-dichloro-4-[4-(dimethylamino)butanoyl]-[1,1′-biphenyl]-3-yl}carbamoyl)-6-hydroxybenzene-1,3-dicarboxylic acid was prepared at the Step 78.1 of Example 78. After preparative HPLC the target compound was selected as the isomer, which matched the NMR spectra.

1H NMR (250 MHz, DMSO-d6) δ 11.42 (d, J=6.5 Hz, 1H), 9.37 (s, 1H), 8.41-8.24 (m, 2H), 8.07 (d, J=8.2 Hz, 1H), 7.83 (d, J=2.1 Hz, 1H), 7.66-7.56 (m, 1H), 7.49 (d, J=8.4 Hz, 1H), 7.37 (d, J=8.2 Hz, 1H), 7.03 (d, J=17.8 Hz, 1H), 3.29-3.04 (m, 4H), 2.79 (d, J=4.2 Hz, 6H), 2.04-1.87 (m, 2H).

### Example 82 3-[2-carboxy-4-(1,2-dihydroxyethyl)benzamido]-3′,4′-difluoro-[1,1′-biphenyl]-4-carboxylic acid (GO-0003780)

3-[2-carboxy-4-(1,2-dihydroxyethyl)benzamido]-3′,4′-difluoro-[1,1′-biphenyl]-4-carboxylic acid was prepared in several steps.

### Step 82.1 Preparation of diisopropyl 4-bromophthalate

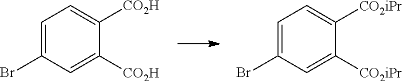

4-Bromophthalic acid (1 g, 4.1 mmol) was esterified with isopropyl alcohol by the standard Fisher esterification using sulfuric acid as a catalyst. Standard workup afforded the diester, diisopropyl 4-bromophthalate (0.974 g, 73% yield) Without further purification, the product was used in the following step.

### Step 82.2 Preparation of diisopropyl 4-vinylphthalate

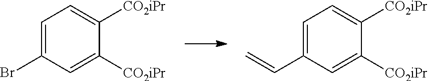

Diisopropyl 4-bromophthalate (0.500 g, 1.51 mmol) was dissolved in DMF (15 mL) and treated with tributyl(vinyl)stannane (0.69 mL, 2.35 mmol) and tetrakis(triphenylphosphine)palladium(0) (0.130 g, 0.11 mmol) in a microwave reactor at 100° C. for 30 min. The cooled reaction mixture was partitioned between water and ethyl acetate and the organic layer washed with water and brine, dried and evaporated to dryness. Flash chromatography (hexane/ethyl acetate 0-50%) afforded pure 4-vinylphthalate (0.432 g).

### Step 82.3 Preparation of diisopropyl 4-(1,2-dihydroxyethyl)phthalate

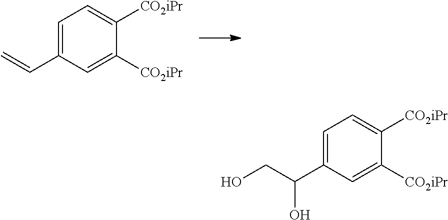

Diisopropyl 4-vinylphthalate (0.400 g, 1.448 mmol) was dissolved in THF/water (4.5 mL:0.45 mL) and treated with osmium tetroxide (460 μL, 4% in water) and morpholine N-oxide (0.204 g, 1.73 mmol) at room temperature for 7 h. The reaction was quenched with saturated Na₂SO₃ solution and then extracted with ethyl acetate (3×). The combined organic layers were washed with water, brine, dried, filtered and concentrated.

Purification by flash chromatography (hexane/ethyl acetate 30-100%) afforded pure diisopropyl 4-(1,2-dihydroxyethyl)phthalate (0.394 g, 83%).

### Step 82.4 Preparation of diisopropyl 4-(1,2-bis(benzyloxy)ethyl)phthalate

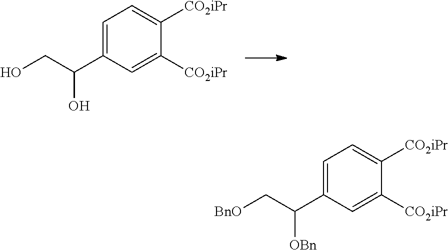

Diisopropyl 4-(1,2-dihydroxyethyl)phthalate (0.250, 0.8 mmol) was dissolved in DMF (6 mL) and treated with sodium hydride (60% in oil, 0.097 g, 2.4 mmol) at room temperature for 30 min. When the bubbling subsided, benzyl bromide (285 μL, 2.4 mmol) was added and the reaction mixture was stirred for an additional 3 h. The reaction was quenched by the addition of saturated ammonium chloride solution and then extracted with ethyl acetate (3×). The combined organic extracts were dried, filtered and concentrated to dryness. The reaction was repeated on a 0.160 g scale and the combined crude products were combined and purified by flash chromatography (hexane/ethyl acetate 0-60%) to afford pure diisopropyl 4-(1,2-bis(benzyloxy)ethyl)phthalate (0.647 g, 87%).

### Step 82.5 Preparation of 4-(1,2-bis(benzyloxy)ethyl)phthalic acid

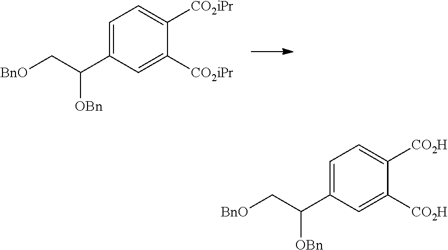

Diisopropyl 4-(1,2-bis(benzyloxy)ethyl)phthalate (0.647 g, 1.31 mmol) was hydrolyzed in the standard way with aqueous sodium hydroxide (3 mL, 2M) in 1:1 methanol/THE (14 mL) at 50° C. for 2.5 h to afford 4-(1,2-bis(benzyloxy)ethyl)phthalic acid (0.576 g) after flash chromatography.

### Step 82.6 Preparation of 3-(5-(1,2-bis(benzyloxy)ethyl)-1,3-dioxoisoindolin-2-yl)-3′,4′-difluoro-[1,1′-biphenyl]-4-carboxylic Acid

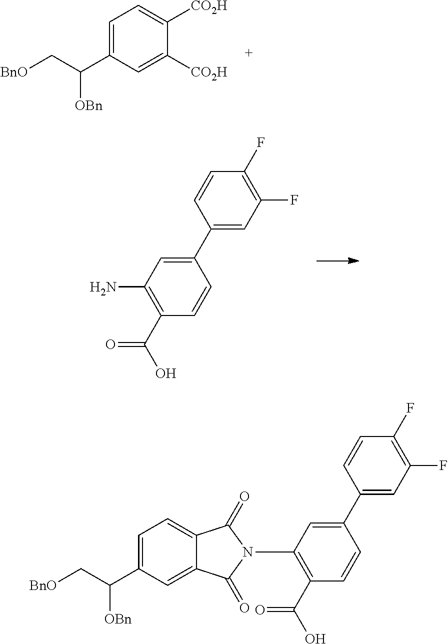 [SMILES: O=C(O)c1ccc(C(COCc2ccccc2)OCc2ccccc2)cc1C(=O)O ; Nc1cc(-c2ccc(F)c(F)c2)ccc1C(=O)O ; O=C(O)c1ccc(-c2ccc(F)c(F)c2)cc1N1C(=O)c2ccc(C(COCc3ccccc3)OCc3ccccc3)cc2C1=O]

4-(1,2-bis(benzyloxy)ethyl)phthalic acid (0.065 g+0.100 g) and 3-amino-3′,4′-difluoro-[1,1′-biphenyl]-4-carboxylic acid (0.040 g+0.62 g) in two separate reactions were reacted in isobutyric acid (2.5 mL+4 mL) in a microwave reactor at 175° C. for 5 h. Solvent was removed and the two reactions were purified together by flash chromatography (hexane/ethyl acetate 0-100%) to afford pure 3-(5-(1,2-bis(benzyloxy)ethyl)-1,3-dioxoisoindolin-2-yl)-3′,4′-difluoro-[1,1′-biphenyl]-4-carboxylic acid (0.164 g, 65% yield).

### Step 82.7 Preparation of 3-(5-(1,2-dihydroxyethyl)-1,3-dioxoisoindolin-2-yl)-3′,4′-difluoro-[1,1′-biphenyl]-4-carboxylic Acid

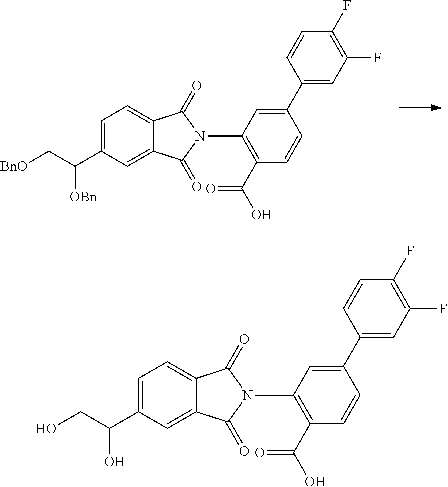 [SMILES: O=C(O)c1ccc(-c2ccc(F)c(F)c2)cc1N1C(=O)c2ccc(C(COCc3ccccc3)OCc3ccccc3)cc2C1=O ; O=C(O)c1ccc(-c2ccc(F)c(F)c2)cc1N1C(=O)c2ccc(C(O)CO)cc2C1=O]

3-(5-(1,2-bis(benzyloxy)ethyl)-1,3-dioxoisoindolin-2-yl)-3′,4′-difluoro-[1,1′-biphenyl]-4-carboxylic acid (0.140 g 0.226 mmol) was dissolved in dichloromethane (10.5 mL) and treated with BCl₃ (2.6 mL, 1 M in DCM) at 0° C. for 40 min. The mixture was diluted with DCM and evaporated to dryness. Preparative HPLC afforded pure 3-(5-(1,2-dihydroxyethyl)-1,3-dioxoisoindolin-2-yl)-3′,4′-difluoro-[1,1′-biphenyl]-4-carboxylic acid.

### Step 82.8 Preparation of 3-(2-carboxy-4-(1,2-dihydroxyethyl)benzamido)-3′,4′-difluoro-[1,1′-biphenyl]-4-carboxylic acid and 3-(2-carboxy-5-(1,2-dihydroxyethyl)benzamido)-3′,4′-difluoro-[1,1′-biphenyl]-4-carboxylic acid

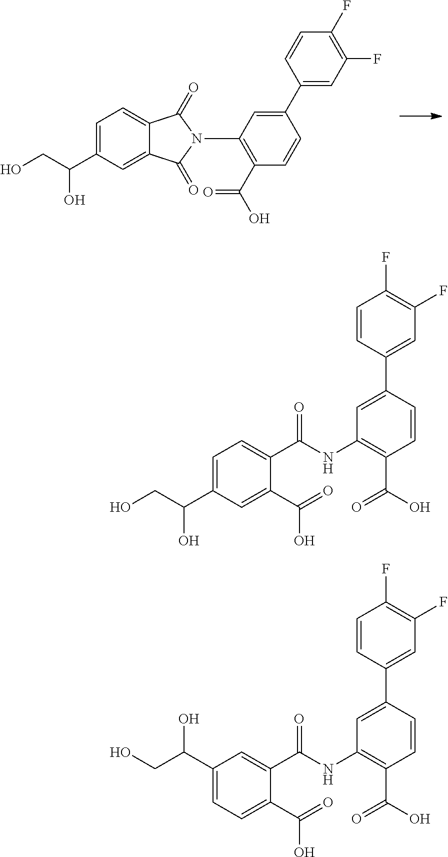 [SMILES: O=C(O)c1ccc(-c2ccc(F)c(F)c2)cc1N1C(=O)c2ccc(C(O)CO)cc2C1=O ; O=C(O)c1ccc(-c2ccc(F)c(F)c2)cc1NC(=O)c1ccc(C(O)CO)cc1C(=O)O ; O=C(O)c1ccc(-c2ccc(F)c(F)c2)cc1NC(=O)c1cc(C(O)CO)ccc1C(=O)O]

Using General Procedure #7, 3-(5-(1,2-dihydroxyethyl)-1,3-dioxoisoindolin-2-yl)-3′,4′-difluoro-[1,1′-biphenyl]-4-carboxylic acid (0.079 g, 0.1798 mmol) was ring-opened with sodium hydroxide solution (0.45 mL, 2M) for 70 min at room temperature, and purified by preparative HPLC to afford pure samples of 3-(2-carboxy-4-(1,2-dihydroxyethyl)benzamido)-3′,4′-difluoro-[1,1′-biphenyl]-4-carboxylic acid and 3-(2-carboxy-5-(1,2-dihydroxyethyl)benzamido)-3′,4′-difluoro-[1,1′-biphenyl]-4-carboxylic acid. After preparative HPLC the target compound was selected as the isomer, which matched the NMR spectra.

1H NMR (250 MHz, DMSO-d6) δ 3.42-3.59 (m, 2H) 4.65 (t, J=5.71 Hz, 1H) 7.48-7.70 (m, 6H) 7.74-7.88 (m, 2H) 8.11 (d, J=8.35 Hz, 1H) 8.95 (s, 1H) 11.61 (s, 1H)

### Example 83: 3-[2-carboxy-5-(1,2-dihydroxyethyl)benzamido]-3′,4′-difluoro-[1,1′-biphenyl]-4-carboxylic acid (GO-0003781)

3-[2-carboxy-5-(1,2-dihydroxyethyl)benzamido]-3′,4′-difluoro-[1,1′-biphenyl]-4-carboxylic acid was prepared at the Step 82.8 of Example 82. After preparative HPLC the target compound was selected as the isomer, which matched the NMR spectra.

1H NMR (250 MHz, DMSO-d6) δ 3.44-3.55 (m, 2H) 4.66 (t, J=6.04 Hz, 1H) 7.52-7.66 (m, 6H) 7.85 (d, J=7.91 Hz, 2H) 8.10 (d, J=8.13 Hz, 1H) 8.94 (s, 1H) 11.60 (s, 1H)

### Example 84 2-{[5-(2,4-dichlorophenyl)-2-oxo-1,2-dihydropyridin-3-yl]carbamoyl}-4-{[dimethyl(oxo)-λ⁶-sulfanylidene]carbamoyl}benzoic acid (GO-0003784)

2-{[5-(2,4-dichlorophenyl)-2-oxo-1,2-dihydropyridin-3-yl]carbamoyl}-4-{[dimethyl(oxo)-λ⁶-sulfanylidene]carbamoyl}benzoic acid was prepared in several steps.

### Step 84.1 Preparation of 2-(benzyloxy)-5-bromo-3-nitropyridine

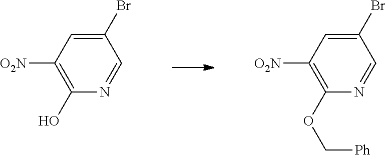 [SMILES: O=[N+]([O-])c1cc(Br)cnc1O ; O=[N+]([O-])c1cc(Br)cnc1OCc1ccccc1]

5-bromo-3-nitropyridin-2-ol (1 g, 4.56 mmol) was protected by reaction with benzyl bromide (0.94 g, 5.48 mmol) and silver carbonate (1.76 g, 6.39 mmol) in toluene at 130° C. for 30 min to afford 2-(benzyloxy)-5-bromo-3-nitropyridine (1.34 g, 95% yield) after flash chromatography (methylene chloride/ethyl acetate 0-50%).

### Step 84.2 Preparation of 2-(benzyloxy)-5-bromopyridin-3-amine

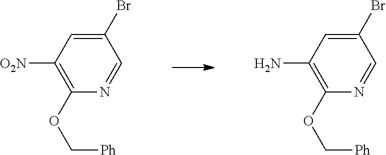 [SMILES: O=[N+]([O-])c1cc(Br)cnc1OCc1ccccc1 ; Nc1cc(Br)cnc1OCc1ccccc1]

2-(benzyloxy)-5-bromo-3-nitropyridine (0.50 g, 1.62 mmol) was reduced by treatment with iron powder (0.41 g, 7.28 mmol) in aqueous ethanol (50%, 3 mL) at 100° C. for 1 h to afford 2-(benzyloxy)-5-bromopyridin-3-amine (0.514 g, 95% yield).

### Step 84.3 Preparation of 2-(benzyloxy)-5-(2,4-dichlorophenyl)pyridin-3-amine

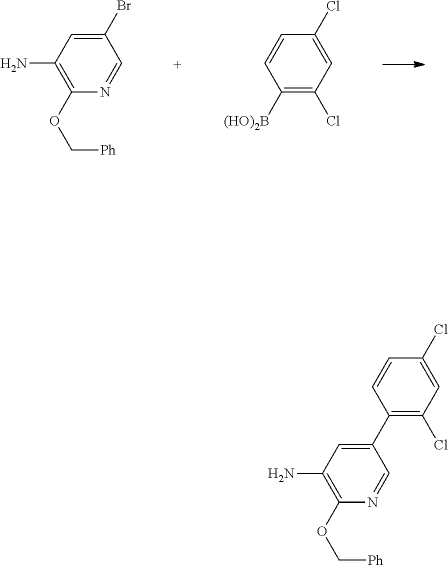 [SMILES: Nc1cc(Br)cnc1OCc1ccccc1 ; OB(O)c1ccc(Cl)cc1Cl ; Nc1cc(-c2ccc(Cl)cc2Cl)cnc1OCc1ccccc1]

The Suzuki coupling of (2,4-dichlorophenyl)boronic acid (0.352 g, 1.84 mmol) and 2-(benzyloxy)-5-bromopyridin-3-amine (0.514 g, 1.84 mmol) was carried out as described in General Procedure #1 in dioxane at 120° C. in a microwave reactor to afford 2-(benzyloxy)-5-(2,4-dichlorophenyl)pyridin-3-amine (0.410 g, 64.4% yield) after flash chromatography (hexane/ethyl acetate 0-70%).

### Step 84.4 Preparation of 2-(2-(benzyloxy)-5-(2,4-dichlorophenyl)pyridin-3-yl)-1,3-dioxoisoindoline-5-carboxylic acid

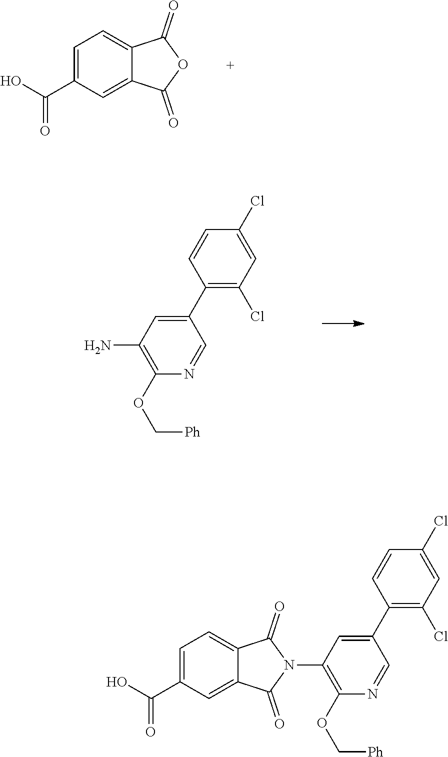 [SMILES: O=C(O)c1ccc2c(c1)C(=O)OC2=O ; Nc1cc(-c2ccc(Cl)cc2Cl)cnc1OCc1ccccc1 ; O=C(O)c1ccc2c(c1)C(=O)N(c1cc(-c3ccc(Cl)cc3Cl)cnc1OCc1ccccc1)C2=O]

1,3-dioxo-1,3-dihydroisobenzofuran-5-carboxylic acid (0.256 g, 1.33 mmol) and 2-(benzyloxy)-5-(2,4-dichlorophenyl)pyridin-3-amine (0.230 g, 0.667 mmol) were reacted in isobutyric acid as described in General procedure #9 to afford 2-(2-(benzyloxy)-5-(2,4-dichlorophenyl)pyridin-3-yl)-1,3-dioxoisoindoline-5-carboxylic acid (0.277 g, 86% yield) after flash chromatography (dichloromethane/methanol 0-20%).

### Step 84.5 Preparation of 2-(2-(benzyloxy)-5-(2,4-dichlorophenyl)pyridin-3-yl)-N-(dimethyl(oxo)-λ6-sulfaneylidene)-1,3-dioxoisoindoline-5-carboxamide

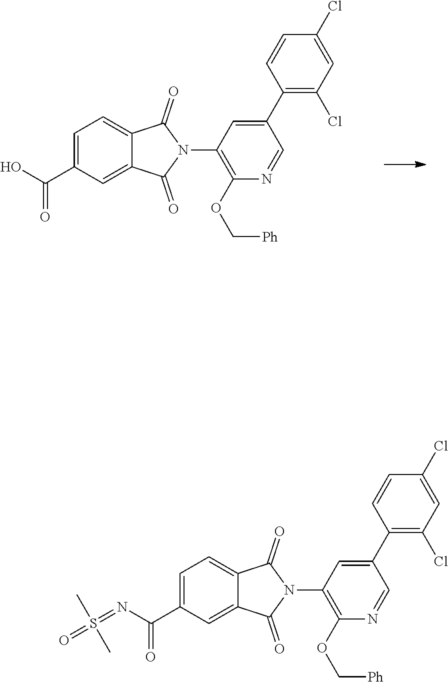 [SMILES: O=C(O)c1ccc2c(c1)C(=O)N(c1cc(-c3ccc(Cl)cc3Cl)cnc1OCc1ccccc1)C2=O ; CS(C)(=O)=NC(=O)c1ccc2c(c1)C(=O)N(c1cc(-c3ccc(Cl)cc3Cl)cnc1OCc1ccccc1)C2=O]

Using General Procedure #11, 2-(2-(benzyloxy)-5-(2,4-dichlorophenyl)pyridin-3-yl)-1,3-dioxoisoindoline-5-carboxylic acid (0.25 g, 0.48 mmol) was converted to 2-(2-(benzyloxy)-5-(2,4-dichlorophenyl)pyridin-3-yl)-N-(dimethyl(oxo)-λ6-sulfaneylidene)-1,3-dioxoisoindoline-5-carboxamide (0.258 g, 90% yield) after flash chromatography (hexane/ethyl acetate 0-100%).

### Step 84.6 Preparation of 2-(5-(2,4-dichlorophenyl)-2-hydroxypyridin-3-yl)-N-(dimethyl(oxo)-λ6-sulfaneylidene)-1,3-dioxoisoindoline-5-carboxamide (and ring-opened side products)

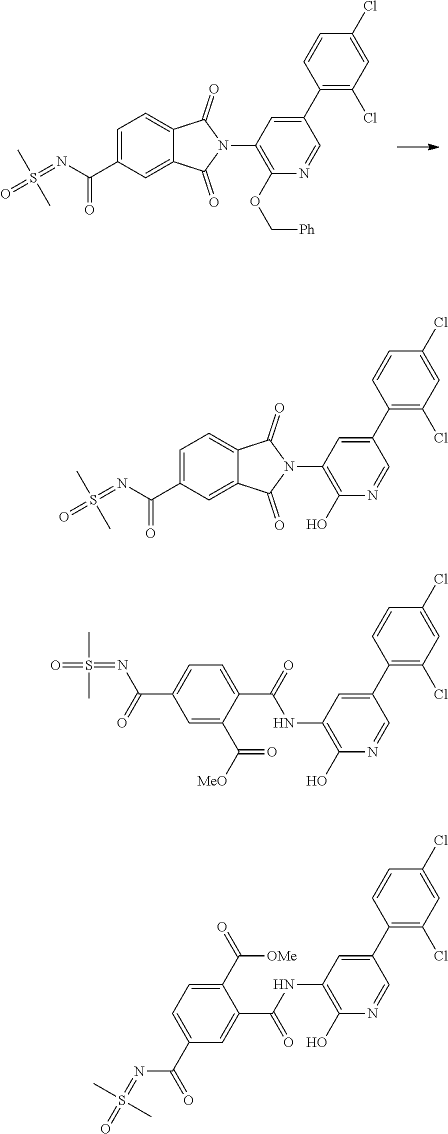 [SMILES: CS(C)(=O)=NC(=O)c1ccc2c(c1)C(=O)N(c1cc(-c3ccc(Cl)cc3Cl)cnc1OCc1ccccc1)C2=O ; CS(C)(=O)=NC(=O)c1ccc2c(c1)C(=O)N(c1cc(-c3ccc(Cl)cc3Cl)cnc1O)C2=O ; COC(=O)c1cc(C(=O)N=S(C)(C)=O)ccc1C(=O)Nc1cc(-c2ccc(Cl)cc2Cl)cnc1O ; COC(=O)c1ccc(C(=O)N=S(C)(C)=O)cc1C(=O)Nc1cc(-c2ccc(Cl)cc2Cl)cnc1O]

2-(2-(benzyloxy)-5-(2,4-dichlorophenyl)pyridin-3-yl)-N-(dimethyl(oxo)-λ⁶-sulfaneylidene)-1,3-dioxoisoindoline-5-carboxamide (0.258 g, 0.434) was hydrogenated at 1 atm hydrogen in 1:1 THE/MeOH (18 mL) using Pd/C (10%, 0.093 g), stirring overnight. This afforded a mixture of 2-(5-(2,4-dichlorophenyl)-2-hydroxypyridin-3-yl)-N-(dimethyl(oxo)-λ6-sulfaneylidene)-1,3-dioxoisoindoline-5-carboxamide as well as two debenzylated ring opened esters. LC/MS confirmed that the chlorines were retained in the reaction. The mixture was not separated and was carried on into the final step.

### Step 84.7 Preparation of 2-((5-(2,4-dichlorophenyl)-2-hydroxypyridin-3-yl)carbamoyl)-5-((dimethyl(oxo)-A6-sulfaneylidene)carbamoyl)benzoic acid and 2-((5-(2,4-dichlorophenyl)-2-hydroxypyridin-3-yl)carbamoyl)-4-((dimethyl(oxo)-λ6-sulfaneylidene)carbamoyl)benzoic acid

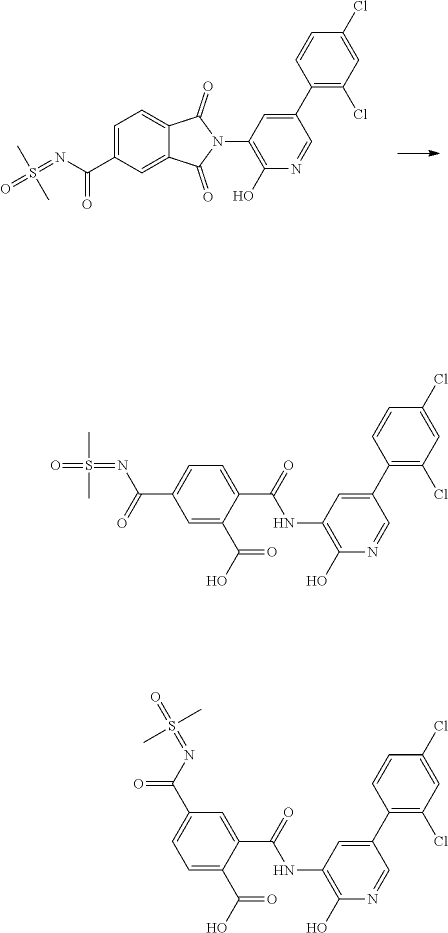 [SMILES: CS(C)(=O)=NC(=O)c1ccc2c(c1)C(=O)N(c1cc(-c3ccc(Cl)cc3Cl)cnc1O)C2=O ; CS(C)(=O)=NC(=O)c1ccc(C(=O)Nc2cc(-c3ccc(Cl)cc3Cl)cnc2O)c(C(=O)O)c1 ; CS(C)(=O)=NC(=O)c1ccc(C(=O)O)c(C(=O)Nc2cc(-c3ccc(Cl)cc3Cl)cnc2O)c1]

The mixture thus obtained in the previous step was hydrolyzed using sodium hydroxide (1.09 mL, 2N) in a 1:1 mixture of THE/methanol (16 mL) at room temperature for 0.5 h to afford 2-((5-(2,4-dichlorophenyl)-2-hydroxypyridin-3-yl)carbamoyl)-5-((dimethyl(oxo)-λ6-sulfaneylidene)-carbamoyl)benzoic acid and 2-((5-(2,4-dichlorophenyl)-2-hydroxypyridin-3-yl)carbamoyl)-4-((dimethyl(oxo)-λ6-sulfaneylidene)carbamoyl)benzoic acid after separation by preparative HPLC. After preparative HPLC the target compound was selected as the isomer, which matched the NMR spectra.

1H NMR (250 MHz, DMSO-d6) δ 3.50 (s, 6H) 7.32 (br. s., 1H) 7.46-7.56 (m, 2H) 7.76 (d, J=1.10 Hz, 1H) 7.91 (d, J=8.13 Hz, 1H) 8.04 (s, 1H) 8.07-8.14 (m, 1H) 8.44 (d, J=1.98 Hz, 1H) 9.63 (s, 1H)

### Example 85 4-{[4-carboxy-2′-(1H-imidazol-4-yl)-[1,1′-biphenyl]-3-yl]carbamoyl}-6-hydroxybenzene-1,3-dicarboxylic acid (GO-0003786)

4-{[4-carboxy-2′-(1H-imidazol-4-yl)-[1,1′-biphenyl]-3-yl]carbamoyl}-6-hydroxybenzene-1,3-dicarboxylic acid was prepared in one step.

### Step 85.1 Preparation of 4-((4-carboxy-2′-(1H-imidazol-5-yl)-[1,1′-biphenyl]-3-yl)carbamoyl)-6-hydroxyisophthalic acid (NSQP00724) and 2-((4-carboxy-2′-(1H-imidazol-5-yl)-[1,1′-biphenyl]-3-yl)carbamoyl)-5-hydroxyterephthalic acid

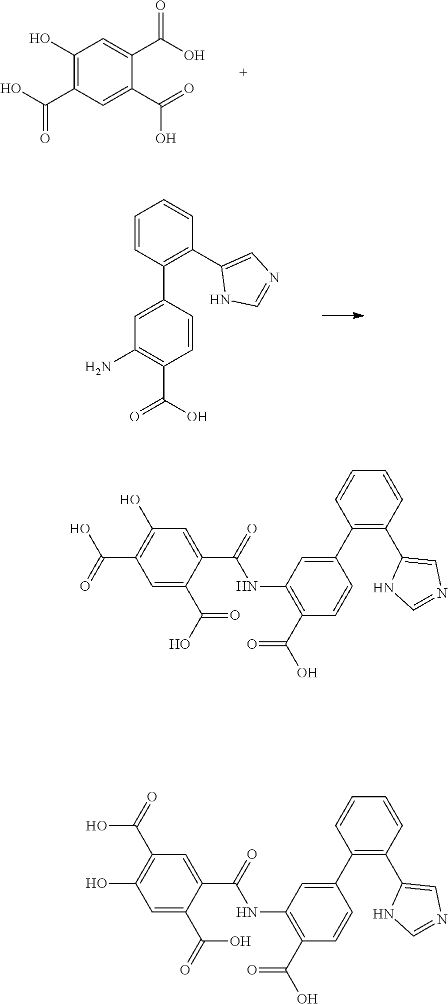 [SMILES: O=C(O)c1cc(C(=O)O)c(C(=O)O)cc1O ; Nc1cc(-c2ccccc2-c2cnc[nH]2)ccc1C(=O)O ; O=C(O)c1cc(C(=O)O)c(C(=O)Nc2cc(-c3ccccc3-c3cnc[nH]3)ccc2C(=O)O)cc1O ; O=C(O)c1cc(C(=O)Nc2cc(-c3ccccc3-c3cnc[nH]3)ccc2C(=O)O)c(C(=O)O)cc1O]

Using a procedure identical to that used for Example 55, 5-hydroxybenzene-1,2,4-tricarboxylic acid (0.160 g) was reacted with 3-amino-2′-(1H-imidazol-5-yl)-[1,1′-biphenyl]-4-carboxylic acid (0.095 g) to afford 4-((4-carboxy-2′-(1H-imidazol-5-yl)-[1,1′-biphenyl]-3-yl)carbamoyl)-6-hydroxyisophthalic acid and 2-((4-carboxy-2′-(1H-imidazol-5-yl)-[1,1′-biphenyl]-3-yl)carbamoyl)-5-hydroxyterephthalic acid after separation and purification by preparative HPLC. After preparative HPLC the target compound was selected as the isomer, which matched the NMR spectra.

1H NMR (250 MHz, DMSO-d6) δ 7.03 (d, J=12.53 Hz, 1H) 7.07-7.18 (m, 1H) 7.41 (s, 1H) 7.54-7.74 (m, 4H) 8.00 (d, J=7.91 Hz, 1H) 8.35 (d, J=3.52 Hz, 1H) 8.37-8.43 (m, 1H) 9.04 (s, 1H)

### Example 86 2-{[4-carboxy-2′-(1H-imidazol-4-yl)-[1,1′-biphenyl]-3-yl]carbamoyl}-5-hydroxybenzene-1,4-dicarboxylic acid (GO-0003787)

2-{[4-carboxy-2′-(1H-imidazol-4-yl)-[1,1′-biphenyl]-3-yl]carbamoyl}-5-hydroxybenzene-1,4-dicarboxylic acid was prepared at the Step 85.1 of Example 85. After preparative HPLC the target compound was selected as the isomer, which matched the NMR spectra.

1H NMR (250 MHz, DMSO-d6) δ 7.04-7.12 (m, 1H) 7.18 (s, 1H) 7.31-7.39 (m, 1H) 7.66 (br. s., 4H) 7.97-8.06 (m, 1H) 8.08-8.16 (m, 1H) 8.49 (br. s., 1H) 8.99 (s, 1H)

The technical problem underlying the present invention was to identify alternative and/or improved means and methods to treat and/or prevent cancer, neurodegeneration, aging and other diseases and conditions, wherein the modulation of PFKFB3/PFKFB4 may have beneficial effect, means and methods of neuroprotection and corresponding methods of manufacturing of medications, kits and other inventions. The solution to this technical problem is achieved by providing the embodiments characterized in this application, including but not limited to the following items.

Accordingly, the present invention also relates to the following items (0) to (1850) and items A-H below:
0. A compound of Formula (I):

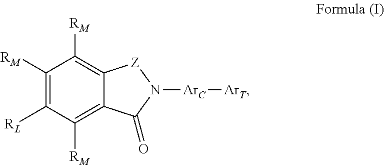

or a pharmaceutically acceptable salt thereof, wherein:
is selected from —C(═O)— and —C(R^{a})(R^{b})—;

R^{a} and R^{b} are independently selected from hydrogen, hydroxyl, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR¹R², —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

Ar_{c} is selected from C₃-C₈ cycloalkenylene, C₂-C₈ heterocycloalkenylene, arylene, and heteroarylene;

wherein Ar_{c} is substituted with one or more R_{c};

each R_{C} are independently selected from —CN, —OH, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, optionally substituted —O—C₂-C₈ heterocycloalkyl, optionally substituted heteroaryl, optionally substituted aryl, —C(═O)OH, —C(═O)OR³, —C(═O)NR⁴R⁵, —S(═O)₂NR⁴R⁵, —NHC(═O)H, —NHC(═O)R⁶, —NHS(═O)₂R⁶, and —C(═O)NHS(═O)₂R⁶;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, —O—C₂-C₈ heterocycloalkyl, aryl, and heteroaryl are optionally substituted with one or more substituents independently selected from —OH, halogen, —C(═O)OR⁷, —C(═O)R⁶, —C(═O)NR¹R², C₁-C₆alkyl, C₁-C₈ alkoxy, C₃-C₈ cycloalkyl, —O—C₃-C₈cycloalkyl, C₂-C₈ heterocycloalkyl, —O—C₂-C₈ heterocycloalkyl, optionally substituted aryl, optionally substituted heteroaryl, and —NR⁷R⁸;

each R¹ and R² is independently selected from hydrogen, optionally substituted C₁-C₆ alkyl, and optionally substituted C₃-C₈ cycloalkyl;

wherein the C₁-C₆ alkyl is optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl is optionally substituted with one or more substituents independently selected from halogen, —C(═O)OR⁷, —C(═O)NR⁷R⁸, —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R³ is independently C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or each R³ is independently C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═OO)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁴ and R⁵ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R⁶ are independently selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁷ and R⁸ is independently selected from hydrogen and C₁-C₆ alkyl;

or R⁷ and R⁸ are taken together with the N to which they are attached to form a C₂-C₈ heterocycloalkyl optionally substituted with one or more C₁-C₆alkyl substituents;

Ar_{T} is selected from pyridinyl, pyrimidinyl, pyridazinyl, pyrazinyl, phenyl, pyrrolyl, furanyl, thiophenyl, pyrazolyl, imidazolyl, triazolyl, and tetrazolyl, wherein Ar_{T} is optionally substituted by one or more substituents independently selected from halogen, —OH, —NR⁷R⁸, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;

each R_{M} is independently selected from hydrogen, halogen, —OH, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from —OH, —CN, optionally substituted C₁-C₆ hydroxyalkyl, optionally substituted C₁-C₆ alkoxy, optionally substituted heteroaryl, —C(═O)OH, —C(═O)OR⁹, —C(═O)NR¹⁰R¹¹, —S(═O)₂NR¹⁰R¹¹, —NHC(═O)H, —NHC(═O)R¹², —NHS(═O)₂R¹² and —C(═O)NHS(═O)₂R¹²;

wherein the C₁-C₆ hydroxyalkyl and C₁-C₆alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the heteroaryl is optionally substituted with one or more of —OH, —O—C(═O)C₁-C₆ alkyl, (C₁-C₄ alkylene)-O—C(═O)C₁-C₆ alkyl, C₁-C₆ alkyl-(aryl), C₁-C₆ alkyl-(heteroaryl), halogen, —C(═O)OR⁷, —C(═O)R¹², —C(═O)NR¹R², C₁-C₆ alkyl, C₁-C₆ hydroxyalkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR¹R²;

R⁹ is C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁹ is C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R¹⁰ and R¹¹ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl (optionally substituted with —OH, halogen, —C(═O)OR⁷, —C(═O)NR⁷R⁸, C₁-C₆ alkyl, C₁-C₆ alkoxy, —NR⁷R⁸), and heteroaryl (optionally substituted with —OH, halogen, —C(═O)OR⁷, —C(═O)NR⁷R⁸, C₁-C₆ alkyl, C₁-C₆ alkoxy, —NR⁷R⁸, C₃-C₈ cycloalkyl, —O—C₃-C₈ cycloalkyl, C₂-C₈ heterocycloalkyl, or —O—C₂-C₈ heterocycloalkyl); and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R¹⁰ and R¹¹ are taken together with the N to which they are attached to form a C₂-C₈ heterocycloalkyl optionally substituted with one or more substituents independently selected from halogen, —OH, C₁-C₆ alkyl, and C₁-C₆ alkoxy;

R¹² is selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

provided that:

(a) at least one of R_{C} is not —NHCOR⁶ when R_{L} is —NHCOR¹² and Ar_{C} is heterocycloalkenylene or heteroarylene; or

(b) at least one of R_{C} is not -Me when R_{L} is —OMe; or

(c) at least one of R_{C} is not —OEt when R_{L} is —C(═O)OH; or

(d) at least one of R_{C} is not —OH when R_{L} is —C(═O)OH; or

(e) at least one of R_{C} is not -Me when R_{L} is —C(═O)OH; or

(f) at least one of R_{C} is not -Et when R_{L} is —OMe; or

(g) at least one of R_{C} is not optionally substituted benzoxazolyl when R_{L} is —C(═O)OH; or

(h) at least one of R_{C} is not optionally substituted isoindoline-1,3-dione when R_{L} is —C(═O)OH.

1. A compound of Formula (0):

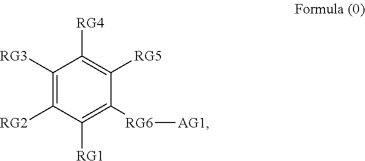

or a pharmaceutically acceptable salt thereof, wherein RG6 and RG5 is one of the following:

A) RG6 and RG5 are taken together with the N to which they are attached to form a C₂-C₈ heterocycloalkyl optionally substituted with one or more substituents;

RG1, RG3 and RG4 are independently selected from R_{M}; RG2 is R_{L}; RG5 is Z; RG6 is —C(═O)—; AG1 is —Ar_{C}—Ar_{T};

thus the Formula (0) can be represented as the Formula (I):

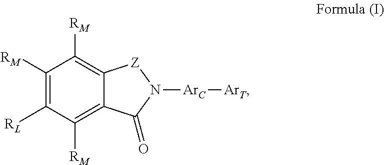

wherein: Z is selected from —C(═O)— and —C(R^{a})(R^{b})—;

R^{a} and R^{b} are independently selected from hydrogen, hydroxyl, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR¹R², —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

Ar_{c} is selected from C₃-C₈ cycloalkenylene, C₂-C₈ heterocycloalkenylene, arylene, and heteroarylene; wherein Ar_{c} is substituted with one or more R_{C};

each R_{C} are independently selected from —CN, —OH, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, optionally substituted —O—C₂-C₈ heterocycloalkyl, optionally substituted heteroaryl, optionally substituted aryl, —C(═O)OH, —C(═O)OR³, —C(═O)NR⁴R⁵, —S(═O)₂NR⁴R⁵, —NHC(═O)H, —NHC(═O)R⁶, —NHS(═O)₂R⁶, and —C(═O)NHS(═O)₂R⁶;

wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, —O—C₂-C₈ heterocycloalkyl, aryl, and heteroaryl are optionally substituted with one or more substituents independently selected from —OH, halogen, —C(═O)OR⁷, —C(═O)R⁶, —C(═O)NR¹R², C₁-C₆alkyl, C₁-C₆alkoxy, C₃-C₈ cycloalkyl, —O—C₃-C₈cycloalkyl, C₂-C₈ heterocycloalkyl, —O—C₂-C₈ heterocycloalkyl, optionally substituted aryl, optionally substituted heteroaryl, and —NR⁷R⁸;

each R¹ and R² is independently selected from hydrogen, optionally substituted C₁-C₆ alkyl, and optionally substituted C₃-C₈ cycloalkyl;

wherein the C₁-C₆ alkyl is optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl is optionally substituted with one or more substituents independently selected from halogen, —C(═O)OR⁷, —C(═O)NR⁷R⁸, —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl;

or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R³ is independently C₁-C₆ alkyl optionally substituted with one or more substituents independently selected from halogen, —OH, optionally substituted —OC(═O)C₁-C₆alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or each R³ is independently C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁴ and R⁵ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R⁶ are independently selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R⁷ and R⁸ is independently selected from hydrogen and C₁-C₆ alkyl;

or R⁷ and R⁸ are taken together with the N to which they are attached to form a C₂-C₈ heterocycloalkyl optionally substituted with one or more C₁-C₆alkyl substituents;

Ar_{T} is selected from pyridinyl, pyrimidinyl, pyridazinyl, pyrazinyl, phenyl, pyrrolyl, furanyl, thiophenyl, pyrazolyl, imidazolyl, triazolyl, and tetrazolyl, wherein Ar_{T} is optionally substituted by one or more substituents independently selected from halogen, —OH, —NR⁷R⁸, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;

each R_{M} is independently selected from hydrogen, halogen, —OH, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from —OH, —CN, optionally substituted C₁-C₆ hydroxyalkyl, optionally substituted C₁-C₆ alkoxy, optionally substituted heteroaryl, —C(═O)OH, —C(═O)OR⁹, —C(═O)NR¹⁰R¹¹, —S(═O)₂NR¹⁰R¹¹, —NHC(═O)H, —NHC(═O)R¹², —NHS(═O)₂R¹² and —C(═O)NHS(═O)₂R¹²;

wherein the C₁-C₆ hydroxyalkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the heteroaryl is optionally substituted with one or more of —OH, —O—C(═O)C₁-C₆ alkyl, (C₁-C₄ alkylene)-O—C(═O)C₁-C₆ alkyl, C₁-C₆ alkyl-(aryl), C₁-C₆ alkyl-(heteroaryl), halogen, —C(═O)OR⁷, —C(═O)R¹², —C(═O)NR¹R², C₁-C₆ alkyl, C₁-C₆ hydroxyalkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR¹R²;

R⁹ is C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;

wherein the —OC(═O)C₁-C₆ alkyl and —C(═O)O—C₁-C₆ alkyl are optionally substituted with one or more substituents independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R⁹ is C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;

wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and

wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

each R¹⁰ and R¹¹ is independently selected from hydrogen, optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl (optionally substituted with —OH, halogen, —C(═O)OR⁷, —C(═O)NR⁷R⁸, C₁-C₆ alkyl, C₁-C₆ alkoxy, —NR⁷R⁸), and heteroaryl (optionally substituted with —OH, halogen, —C(═O)OR⁷, —C(═O)NR⁷R⁸, C₁-C₆ alkyl, C₁-C₆ alkoxy, —NR⁷R⁸, C₃-C₈ cycloalkyl, —O—C₃-C₈ cycloalkyl, C₂-C₈ heterocycloalkyl, or —O—C₂-C₈ heterocycloalkyl); and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

or R¹⁰ and R¹¹ are taken together with the N to which they are attached to form a C₂-C₈ heterocycloalkyl optionally substituted with one or more substituents independently selected from halogen, —OH, C₁-C₆ alkyl, and C₁-C₆ alkoxy;

R¹² is selected from optionally substituted C₁-C₆ alkyl, optionally substituted C₃-C₈ cycloalkyl, optionally substituted aryl, and optionally substituted heteroaryl;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;

provided that:

(i) at least one of R_{C} is not —NHCOR⁶ when R_{L} is —NHCOR¹² and Ar_{C} is heterocycloalkenylene or heteroarylene; or

(j) at least one of R_{C} is not -Me when R_{L} is —OMe; or

(k) at least one of R_{C} is not —OEt when R_{L} is —C(═O)OH; or

(l) at least one of R_{C} is not —OH when R_{L} is —C(═O)OH; or

(m) at least one of R_{C} is not -Me when R_{L} is —C(═O)OH; or

(n) at least one of R_{C} is not -Et when R_{L} is —OMe; or

(o) at least one of R_{C} is not optionally substituted benzoxazolyl when R_{L} is —C(═O)OH; or

(p) at least one of R_{C} is not optionally substituted isoindoline-1,3-dione when R_{L} is —C(═O)OH.

B) RG6 and RG5 do not form a C₂-C₈ heterocycloalkyl; RG1 is R5; RG2 is R1; RG3 is R6; RG4 is R20; RG5 is R4; RG6 is R10; AG1 is A;

thus the Formula (0) can be represented as the Formula (VII):

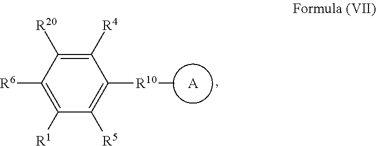

or a pharmaceutically acceptable salt thereof, wherein:

A is selected from:

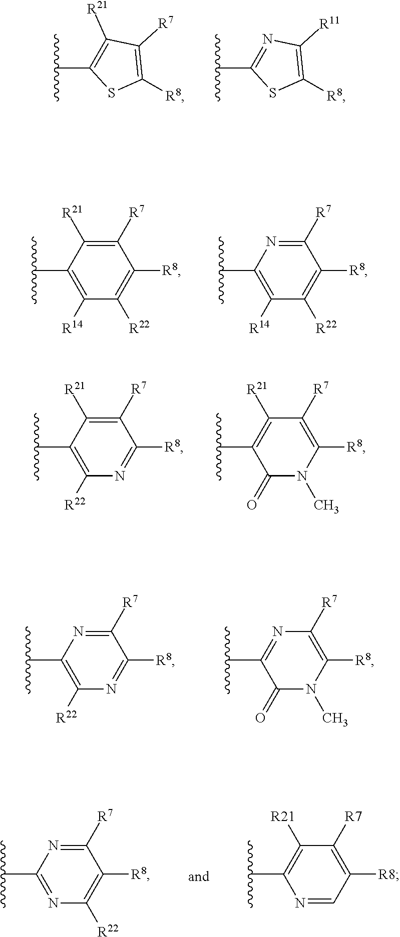

R¹ is selected from hydrogen, halogen, hydroxyl, C₁-C₆ alkyl, and C₁-C₆ alkoxy, wherein the C₁-C₆ alkyl and C₁-C₆ alkoxy are optionally substituted with one or more halogens; each R² and R³ is independently selected from hydrogen and C₁-C₆ alkyl, wherein the C₁-C₆ alkyl is optionally substituted with one or more halogens;

or R² and R³ are taken together with the N to which they are attached to form a 3- to 10-membered heterocycle optionally substituted with one or more substituents independently selected from C₁-C₆ alkyl;

R⁴ is selected from hydrogen, halogen, C₁-C₆ alkyl, and C₁-C₆alkoxy,

wherein the C₁-C₆ alkyl and C₁-C₆ alkoxy are optionally substituted with one or more substituents independently selected from halogens;

R⁵ is selected from —C(═O)OR¹⁵, —C(═O)NR²R³, —S(═O)₂NR²R³, —C(═O)NHR¹⁵, —CH₂OH, 3-hydroxyoxetan-3-yl, and —NH₂;

R⁶ is selected from hydrogen, halogen, hydroxyl, 5-membered heteroaryl, C₁-C₆ alkyl, —C(═O)OR¹⁵, —C(═O)R¹², —C(═O)NHR¹⁵, and —C(═O)N═S(═X³)(CH₃)₂,

wherein the C₁-C₆ alkyl are optionally substituted with one or more R⁹, and

wherein 5-membered heteroaryl contains at least two heteroatoms and is optionally substituted with one or more substituents independently selected from R¹⁷;

R⁷ is selected from hydrogen, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,

wherein the C₁-C₆ alkyl and C₁-C₆ alkoxy are optionally substituted with one or more halogens, and

wherein C₃-C₈ cycloalkyl, —O—C₃-C₈ cycloalkyl, 3- to 10-membered heterocycloalkyl, —O—(3- to 10-membered heterocycloalkyl), aryl and heteroaryl are optionally substituted with one or more R²⁴;

R⁸ is selected from hydrogen, —NO₂, C₁-C₆ alkyl, aryl, and heteroaryl,

wherein the C₁-C₆ alkyl and C₁-C₆ alkoxy are optionally substituted with one or more substituents independently selected at each occurrence from halogen; and

wherein aryl and heteroaryl are optionally substituted with one or more substituents independently selected at each occurrence from R²³;

or R⁷ and R⁸ are taken together to form a C₅-C₁₀ carbocycle or 5- to 10-membered heterocycle,

wherein C₅-C₁₀ carbocycle and 5- to 10-membered heterocycle are optionally substituted with one or more substituents independently selected from halogen, hydroxyl, —NO₂, C₁-C₆ alkyl, C₁-C₆ alkoxy, C₃-C₈ cycloalkyl, —O—C₃-C₈cycloalkyl, 3- to 10-membered heterocycloalkyl, —O—(3- to 10-membered heterocycloalkyl), aryl, and heteroaryl,

wherein the C₁-C₆ alkyl and C₁-C₆ alkoxy are optionally substituted with one or more halogens, and

wherein aryl, heteroaryl, C₃-C₈ cycloalkyl, —O—C₃-C₈ cycloalkyl, 3- to 10-membered heterocycloalkyl, and —O-(3- to 10-membered heterocycloalkyl) are optionally substituted with one or more R²³;

each R⁹ is independently selected from hydroxy and —COOH;

R¹⁰ is selected from —C(═O)—X¹—, —CH₂—X¹—, —X¹—C(═O)—, and —X¹—CH₂—;

R¹¹ is selected from hydrogen, —NO₂, C₁-C₆ alkyl, C₁-C₆alkoxy, C₃-C₈ cycloalkyl, —O—C₃-C₈ cycloalkyl, 3- to 10-membered heterocycloalkyl, and —O—(3- to 10-membered heterocycloalkyl),

wherein the C₁-C₆ alkyl and C₁-C₆ alkoxy are optionally substituted with one or more halogens, and

wherein C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;

R¹² is selected from alanine, arginine, asparagine, aspartic acid, asparagine, aspartic acid, cysteine, glutamic acid, glutamine, glutamine, glutamic acid, glycine, histidine, isoleucine, leucine, lysine, methionine, phenylalanine, proline, serine, threonine, tryptophan, tyrosine, and valine, wherein the point of attachment of R¹² is a nitrogen atom;

R¹⁴ is selected from hydrogen, halogen, hydroxyl, nitrile, —C(═O)CR¹⁵ and —C(═O)OR¹⁵;

each R¹⁵ is independently selected from hydrogen and C₁-C₆ alkyl, -heterocyclyl,

wherein the C₁-C₆ alkyl is optionally substituted with one or more substituents independently selected at each occurrence from —C(═O)NR²R³, -heterocyclyl, —NR²R³;

wherein the heterocyclyl is optionally substituted with one or more substituents independently selected at each occurrence from R² and R³.

R¹⁷ is selected from C₁-C₆ alkyl, aryl, and 6-membered heteroaryl,

wherein the C₁-C₆ alkyl is optionally substituted with one or more hydroxy, and

wherein aryl and 6-membered heteroaryl are optionally substituted with one or more substituents independently selected from halogen, —R², and —OR²;

R²⁰ is selected from hydrogen, halogen, hydroxyl, —COOH, —NC(═O)R², —OR², 5-membered heteroaryl, C₁-C₆ alkyl, —C(═O)N═S(═X³)(CH₃)₂, —CH₂(OH)CH₂OH and —NH—SO₂—R²,

wherein the 5-membered heteroaryl contains at least two heteroatoms, and

wherein the C₁-C₆ alkyl is optionally substituted with one or more substituents independently selected from 5-membered heteroaryl, wherein the 5-membered heteroaryl contains at least two heteroatoms;

R²¹ is selected from hydrogen and nitrile;

R²² is selected from hydrogen and hydroxy;

each R²³ is independently selected from halogen, C₁-C₆ alkyl, C₁-C₆ alkoxy,

wherein the C₁-C₆ alkyl and C₁-C₆ alkoxy are optionally substituted with one or more substituents independently selected from halogens;

each R²⁴ is independently selected from halogen, C₁-C₆ alkyl, C₁-C₆ alkoxy, 5-membered heteroaryl wherein the C₁-C₆ alkyl and C₁-C₆ alkoxy are optionally substituted with one or more substituents independently selected from halogens;

each X¹ is independently selected from —NR²— and —CR²R³—; and

each X³ is independently selected from NH and O. re8

2. The compound of item 1, wherein Z is —C(═O)—.

3. The compound of item 1, wherein Z is —C(R^{a})(R^{b})—, and R^{a} and R^{b} are each independently selected from hydrogen, halogen, —OH, C₁₋₆ alkyl, and C₁₋₆ alkoxy.

4. The compound of item 1 or 3, wherein Z is —C(R^{a})(R^{b})—, and R^{a} and R^{b} are each independently selected from hydrogen, fluorine, and methyl.

5. The compound of any one of items 1, 3, or 4, wherein Z is —CH₂—.

6. The compound of any one of items 1-5, wherein Ar_{C} is arylene or heteroarylene; each substituted with one or more R_{C}.

7. The compound of any one of items 1-6, wherein Ar_{C} is arylene substituted with one or two R_{C}.

8. The compound of any one of items 1-6, wherein Ar_{C} is a phenylene substituted with one or two R_{C}.

9. The compound of any one of items 1-6, wherein Ar_{C} is arylene substituted with one R_{C}.

10. The compound of item 9, wherein Ar_{C} is phenylene substituted with one R_{C}.

11. The compound of any one of items 1-6, wherein Ar_{C} is heteroarylene substituted with one or two R_{C}.

12. The compound of any one of items 1-6, wherein Ar_{C} is a monocyclic heteroarylene substituted with one or two R_{C}.

13. The compound of any one of items 1-6, wherein Ar_{C} is heteroarylene substituted with one R_{C}.

14. The compound of item 11, wherein Ar_{C} is thiophenylene substituted with one R_{C}.

15. The compound of item 11, wherein Ar_{C} is thiophenylene substituted with two R_{C}.

16. The compound of any one of items 1-15, wherein each R_{C} are independently selected from —CN, —OH, halogen, optionally substituted C₁-C₆ alkyl, optionally substituted C₃-C₈ cycloalkyl, optionally substituted C₁-C₆ alkoxy, optionally substituted heteroaryl, optionally substituted aryl, —C(═O)OH, —C(═O)OR³, and —C(═O)NR⁴R⁵;

wherein the C₁-C₆ alkyl and C₁-C₆ alkoxy are optionally substituted with one or more substituent independently selected from halogen, —C(═O)OR⁷, —C(═O)NR¹R², —OH, aryl, heteroaryl, C₁-C₆ alkoxy, C₃-C₈ cycloalkyl, and C₂-C₈ heterocycloalkyl; and

wherein the C₃-C₈ cycloalkyl, aryl and heteroaryl are optionally substituted with one or more substituents independently selected from —OH, halogen, —C(═O)OR⁷, —C(═O)NR¹R², C₁-C₆alkyl, C₁-C₈ alkoxy, and —NR⁷R⁸.

17. The compound of any one of items 1-16, wherein each R_{C} are independently selected from —CN, —OH, halogen, C₁-C₆ alkyl, C₁-C₆ hydroxyalkyl, C₁-C₆ hydroxycycloalkyl, C₁-C₆ alkoxy, heteroaryl, aryl, —C(═O)OH, —C(═O)OR³, and —C(═O)NR⁴R⁵.

18. The compound of any one of items 1-8, 11, 12, 15, wherein one R_{C} is selected from —CN, —OH, halogen, C₁-C₆ alkyl, C₁-C₆ hydroxyalkyl, C₁-C₆ hydroxycycloalkyl, C₁-C₆ alkoxy, heteroaryl, aryl, —C(═O)OH, —C(═O)OR³, and —C(═O)NR⁴R⁵; and a second R_{C} is selected from —OH, halogen, C₁-C₆ alkyl, C₁-C₆ alkoxy, or aryl.

19. The compound of any one of items 1-15, wherein each R_{C} are independently selected from —CN, —C(═O)OH, —C(═O)OR³, and tetrazolyl.

20. The compound of any one of items 1-8, 11, 12, 15, wherein one R_{C} is selected from —CN, —C(═O)OH, —C(═O)OR³, and tetrazolyl; and a second R_{C} is selected from —OH, halogen, C₁-C₆ alkyl, C₁-C₆ alkoxy, or aryl.

21. The compound of any one of items 1-20, wherein each R³ is independently selected from C₁-C₆ alkyl optionally substituted with one or more of —OH, optionally substituted —OC(═O)C₁-C₆ alkyl, C₁-C₆ alkoxy, —C(═O)OH, and —NR¹R²; wherein the —OC(═O)C₁-C₈ alkyl is optionally substituted with one or more of —OH and —NR⁷R⁸.

22. The compound of any one of items 1-21, wherein each R³ is independently selected from C₁-C₆ alkyl (optionally substituted with one or more of —OH, C₁-C₈ alkoxy, and —NR¹R²) or —C₁-C₆alkylene-OC(═O)C₁-C₆ alkyl (wherein C₁-C₆ alkyl is optionally substituted with one or more of —OH and —NR⁷R⁸).

23. The compound of any one of items 1-22, wherein each R³ is independently C₁-C₆ alkyl optionally substituted with one or more of —OH, C₁-C₆ alkoxy, and —NR¹R².

24. The compound of any one of items 1-21, wherein each R³ is independently selected from

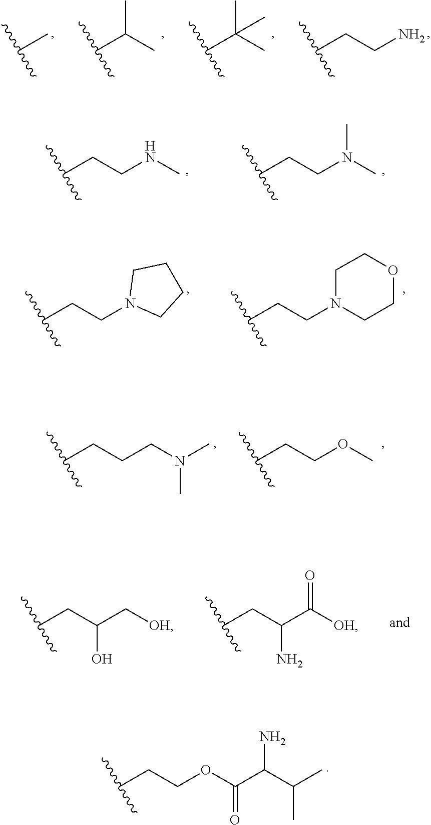

25. The compound of any one of items 16-18, wherein each R⁴ and R⁵ is independently selected from hydrogen and C₁-C₆ alkyl; or R⁴ and R⁵ are taken together with the N to which they are attached to form a C₂-C₈ heterocycloalkyl, optionally substituted with one or more C₁-C₆ alkyl substituents.

26. The compound of any one of items 16-18 and 25, wherein each R⁴ and R⁵ are hydrogen.

27. The compound of any one of items 1-20, wherein at least one of R_{C} is —CN.

28. The compound of any one of items 1-20, wherein at least one of R_{C} is —C(═O)OH.

29. The compound of any one of items 1-20, wherein at least one of R_{C} is tetrazolyl.

30. The compounds of any one of items 1-29, wherein Ar_{T} is selected from pyridinyl, pyrimidinyl, pyrazinyl, phenyl, thiophenyl, pyrazolyl, and imidazolyl, wherein Ar_{T} is optionally substituted by one or more substituents independently selected from halogen, —OH, —NR⁷R⁸, —CN, C₁-C₆ alkyl, and C₁-C₆ alkoxy.

31. The compounds of any one of items 1-29, wherein Ar_{T} is phenyl optionally substituted by one or more substituents selected from halogen, —OH, —NR⁷R⁸, —CN, C₁-C₆alkyl, and C₁-C₆alkoxy.

32. The compound of any one of items 1-31, wherein each R_{M} is independently selected from hydrogen, halogen, —OH, —CN, C₁-C₆alkyl, and C₁-C₆alkoxy.

33. The compound of any one of items 1-32, wherein one R_{M} is selected from hydrogen, halogen, —OH, —CN, C₁-C₆ alkyl, and C₁-C₆ alkoxy; and each other R_{M} is independently selected from hydrogen and halogen.

34. The compound of any one of items 1-33, wherein each R_{M} is hydrogen.

35. The compound of any one of items 1-34, wherein R_{L} is selected from optionally substituted heteroaryl, —C(═O)OH, —C(═O)OR⁹, —C(═O)NR¹⁰R¹¹, —NHC(═O)R¹², —NHS(═O)₂R¹², and —C(═O)NHS(═O)₂R¹²;

wherein the heteroaryl is optionally substituted with one or more substituents independently selected from —OH, C₁-C₆ alkyl, C₁-C₆ hydroxyalkyl, C₁-C₆ alkoxy, —OC(═O)C₁-C₆ alkyl, (C₁-C₄ alkylene)-O—C(═O)C₁-C₆ alkyl, —C(═O)R¹², aryl, heteroaryl, C₁-C₆ alkyl-(aryl), and C₁-C₆ alkyl-(heteroaryl).

36. The compound of item 35, wherein R_{L} is —C(═O)OR⁹.

37. The compound of item 36, wherein R⁹ is C₁-C₆alkylene-OC(═O)C₁-C₆alkyl, wherein C₁-C₆alkyl is optionally substituted with one or more of —OH and —NR⁷R⁸.

38. The compound of item 36, wherein R⁹ is C₁-C₆ alkyl optionally substituted with —NR¹R².

39. The compound of item 38, wherein each R¹ and R² is independently selected from hydrogen or C₁-C₆ alkyl.

40. The compound of items 38 or 39, wherein R⁹ is selected from

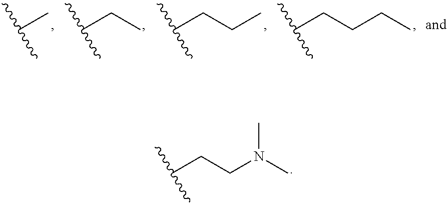

41. The compound of item 35, wherein R_{L} is —C(═O)NR¹⁰R¹¹, and each R¹⁰ and R¹¹ is independently selected from hydrogen and C₁-C₆ alkyl optionally substituted with one or more substituents independently selected from —C(═O)OH, —C(═O)NR¹R², —OH, aryl, and heteroaryl; or R¹⁰ and R¹¹ are taken together with the N to which they are attached to form a C₂-C₈ heterocycloalkyl, optionally substituted with one or more C₁-C₆ alkyl substituents.

42. The compound of item 35 or 41, wherein R_{L} is —C(═O)NR¹⁰R¹¹; R¹⁰ is hydrogen; and R¹¹ is selected from hydrogen

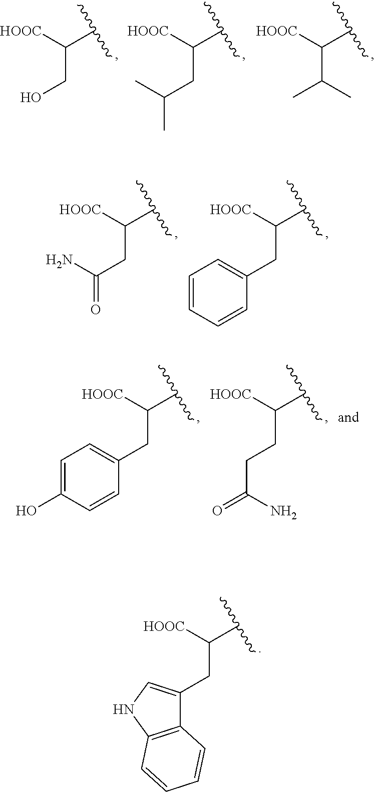

43. The compound of item 35, wherein R_{L} is selected from —NHC(═O)R¹², —NHS(═O)₂R¹², and —C(═O)NHS(═O)₂R¹², and R¹² is selected from C₁-C₆ alkyl and aryl optionally substituted with one or more C₁-C₆ alkyl substituents.

44. The compound of item 43, wherein R_{L} is —NHC(═O)R¹²; and R¹² is methyl.

45. The compound of item 43, wherein R_{L} is —NHS(═O)₂R¹²; and R¹² is selected from phenyl, tolyl, and methyl.

46. The compound of item 43, wherein R_{L} is —C(═O)NHS(═O)₂R¹²; and R¹² is selected from methyl, butyl, and phenyl.

47. The compound of item 35, wherein R_{L} is —C(═O)OH.

48. The compound of item 35, wherein R^{L} is monocyclic heteroaryl, optionally substituted with one or more substituents independently selected from —OH, C₁-C₆ alkyl, C₁-C₆ hydroxyalkyl, C₁-C₆ alkoxy, —OC(═O)C₁-C₆ alkyl, (C₁-C₄ alkylene)-O—C(═O)C₁-C₆ alkyl, —C(═O)R¹², aryl, heteroaryl, C₁-C₆ alkyl-(aryl), and C₁-C₆ alkyl-(heteroaryl).

49. The compound of item 35 or 48, wherein R_{L} is tetrazolyl.

50. The compound of item 35 or 48, wherein R_{L} is triazolyl, optionally substituted with one or more substituents independently selected from —OH, C₁-C₆ alkyl, C₁-C₆ hydroxyalkyl, C₁-C₆ alkoxy, —OC(═O)C₁-C₆ alkyl, (C₁-C₄ alkylene)-O—C(═O)C₁-C₆ alkyl, —C(═O)R¹², aryl, heteroaryl, C₁-C₆ alkyl-(aryl), and C₁-C₆ alkyl-(heteroaryl).

51. The compound of item 35, 48 or 50, wherein R_{L} is triazolyl.

52. The compound of any one of items 1-51, wherein each R¹ and R² is independently selected from hydrogen and C₁-C₆ alkyl; or R¹ and R² are taken together with the N to which they are attached to form a C₂-C₈ heterocycloalkyl, optionally substituted with one or more C₁-C₆ alkyl substituents.

53. The compound of any one of items 1-52, wherein each R¹, R², R⁷ and R⁸ is independently selected from hydrogen and C₁-C₆ alkyl.

54. The compound of item 53, wherein each R¹ and R⁸ is hydrogen and each R² and R⁷ is independently selected from hydrogen and C₁-C₆ alkyl.

55. The compound of item 53 or 54, wherein R¹, R², R⁷ and R⁸ are each hydrogen.

56. The compound of any one of items 1-55, wherein the compound or a pharmaceutically acceptable salt thereof is in the form of a prodrug.

57. The compound of item 56, wherein the prodrug comprises an ester moiety.

58. The compound of item 56, wherein the prodrug comprises an amide moiety.

59. The compound of item 1, wherein the compound of Formula (I) is represented by Formula (Ia) or Formula (Ib):

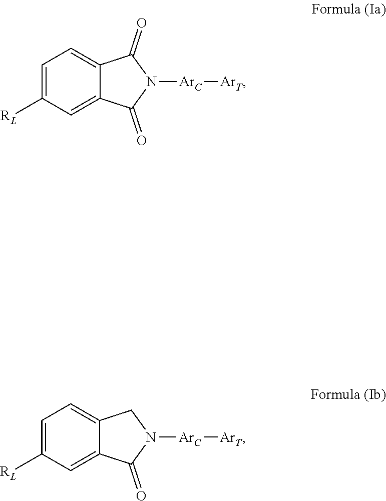

or a pharmaceutically acceptable salt thereof wherein:

Ar_{C} is selected from arylene and heteroarylene; wherein Ar_{C} is substituted with one or more R_{C};

each R_{C} are independently selected from halogen, —CN, optionally substituted C₁-C₆ alkyl, optionally substituted C₁-C₆ alkoxy, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

wherein the C₁-C₆ alkyl and C₁-C₆ alkoxy are optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl; and

wherein the aryl and heteroaryl are optionally substituted with one or more substituents independently selected from —OH, halogen, —C(═O)OH, —C(═O)NR¹R², C₁-C₆ alkyl, C₁-C₆ alkoxy, and —NR⁷R⁸;

each R¹ and R² is independently selected from hydrogen and C₁-C₆ alkyl optionally substituted with one or more substituents independently selected from halogen, —C(═O)OH, —C(═O)NR⁷R⁸, —OH, aryl, heteroaryl, C₃-C₈ cycloalkyl, and C₂-C₈ heterocycloalkyl;

or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R³ is independently C₁-C₆ alkyl optionally substituted with one or more substituents independently selected from —OH, optionally substituted —OC(═O)C₁-C₆ alkyl, optionally substituted —C(═O)OC₁-C₆ alkyl, C₁-C₆ alkoxy, —C(═O)OH, and —NR¹R²; wherein the —OC(═O)C₁-C₆ alkyl and —C(═O)OC₁-C₆ alkyl are optionally substituted with one or more substituents independently selected from —OH and —NR⁷R⁸;

each R⁴ and R⁵ is independently selected from hydrogen and C₁-C₆ alkyl optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl;

or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;

each R⁶ are independently selected from optionally substituted C₁-C₆ alkyl and optionally substituted aryl;

wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and

wherein the aryl is optionally substituted with one or more substituents independently selected from —OH, halogen, —C(═O)OR⁷, —C(═O)NR⁷R⁸, C₁-C₆ alkyl, C₁-C₆ alkoxy, and —NR⁷R⁸;

each R⁷ and R⁸ is independently selected from hydrogen and C₁-C₆ alkyl;

or R⁷ and R⁸ are taken together with the N to which they are attached to form an optionally substituted C₂-C₈ heterocycloalkyl optionally substituted with one or more C₁-C₆ alkyl substituents;

Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²; wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹R², —C(═O)R¹², aryl, and C₁-C₆ alkyl-(aryl);

R⁹ is C₁-C₆ alkyl optionally substituted with one or more substituent independently selected from —OH and —NR¹R²;

each R¹⁰ and R¹¹ is independently selected from hydrogen and C₁-C₆ alkyl optionally substituted with one or more substituents independently selected from —C(═O)OH, —C(═O)NR¹R², —OH, aryl, hydroxyaryl and heteroaryl;

or R¹⁰ and R¹¹ are taken together with the N to which they are attached to form a C₂-C₈ heterocycloalkyl optionally substituted with one or more C₁-C₆ alkyl substituents;

R¹² is selected from C₁-C₆ alkyl and aryl optionally substituted with one or more C₁-C₆ alkyl substituents;

provided that at least one of R_{C} is not —OH when R_{L} is —C(═O)OH in Formula (Ia) or at least one of R_{C} is not —OEt when R_{L} is —C(═O)OH in Formula (Ia).

60. The compound of item 59, wherein Ar_{C} is arylene substituted with one or two R_{C}.

61. The compound of items 59 or 60, wherein Ar_{C} is a monocyclic arylene substituted with one or two R_{C}.

62. The compound of items 59 or 60, wherein Ar_{C} is arylene substituted with one R_{C}.

63. The compound of item 62, wherein Ar_{C} is phenylene substituted with one R_{C}.

64. The compound of items 59, wherein Ar_{C} is heteroarylene substituted with one or two R_{C}.

65. The compound of items 59 or 64, wherein Ar_{C} is a monocyclic heteroarylene substituted with one or two R_{C}.

66. The compound of items 59 or 64, wherein Ar_{C} is heteroarylene substituted with one R_{C}.

67. The compound of item 64, wherein Ar_{C} is thiophenylene substituted with one R_{C}.

68. The compound of item 64, wherein Ar_{C} is thiophenylene substituted with two R_{C}.

69. The compound of any one of items 59-68, wherein each R_{C} are independently selected from —OH, —CN, halogen, C₁-C₆ alkyl, C₁-C₆ alkoxy, C₁-C₆ hydroxyalkyl, heteroaryl, aryl, —C(═O)OH, —C(═O)OR³, and —C(═O)NR⁴R⁵.

70. The compound of any one of items 59-68, wherein each R_{C} are independently selected from —CN, halogen, C₁-C₆ alkyl, C₁-C₆ hydroxyalkyl, heteroaryl, aryl, —C(═O)OH, —C(═O)OR³, and —C(═O)NR⁴R⁵.

71. The compound of any one of items 59, 60, 61, 64, 65 or 68, wherein one R_{C} is selected from —OH, —CN, C₁-C₆ hydroxyalkyl, heteroaryl, aryl, —C(═O)OH, —C(═O)OR³, and —C(═O)NR⁴R⁵; and a second R_{C} is selected from —OH, halogen, C₁-C₆ alkyl, C₁-C₆ alkoxy, and aryl.

72. The compound of any one of items 59-68, wherein each R_{C} are independently selected from —CN, —C(═O)OH, —C(═O)OR³, and tetrazolyl.

73. The compound of any one of items 59, 60, 61, 64, 65 or 68, wherein one R_{C} is selected from —CN, —C(═O)OH, —C(═O)OR³, and tetrazolyl; and a second R_{C} is selected from —OH, halogen, C₁-C₆ alkyl, C₁-C₆ alkoxy, and aryl.

74. The compound of any one of items 59-73, wherein each R³ is independently C₁-C₆ alkyl optionally substituted with one or more substituent selected from —OH, optionally substituted —OC(═O)C₁-C₆ alkyl, optionally substituted —C(═O)OC₁-C₆ alkyl, C₁-C₆ alkoxy, —C(═O)OH, —NR¹R²; wherein the —OC(═O)C₁-C₆ alkyl and —C(═O)OC₁-C₆ alkyl are optionally substituted with one or more substituent independently selected from —OH and —NR⁷R⁸.

75. The compound of any one of items 59-74, wherein each R³ is independently C₁-C₆ alkyl optionally substituted with one or more substituents independently selected from C₁-C₆ alkoxy and —NR¹R².

76. The compound of any one of items 59-74, wherein each R³ is independently selected from

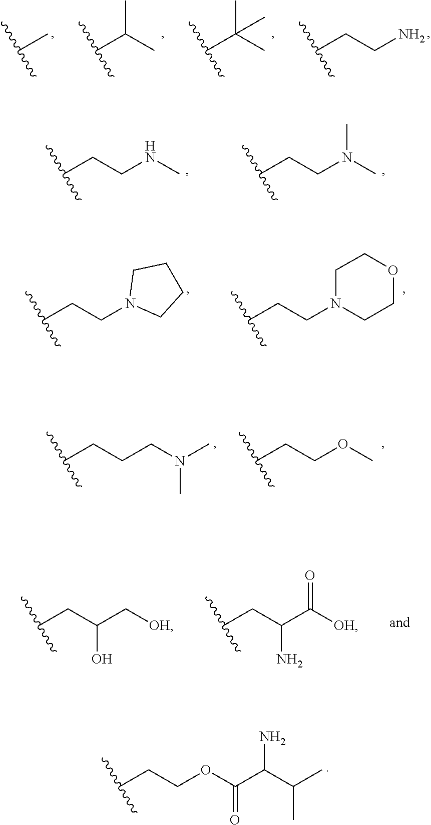

77. The compound of any one of items 69-71, wherein each R⁴ and R⁵ is independently selected from hydrogen and C₁-C₆ alkyl; or R⁴ and R⁵ are taken together with the N to which they are attached to form a C₂-C₈ heterocycloalkyl, optionally substituted with one or more C₁-C₆ alkyl substituents.

78. The compound of any one of items 69-71 or 77, wherein each R⁴ and R⁵ is hydrogen.

79. The compound of any one of items 59-73, wherein at least one of R_{C} is —CN.

80. The compound of any one of items 59-73, wherein at least one of R_{C} is —C(═O)OH.

81. The compound of any one of items 59-73, wherein at least one of R_{C} is tetrazolyl.

82. The compounds of any one of items 59-81, wherein Ar_{T} is phenyl optionally substituted by one or more substituents independently selected from halogen, —OH, —NR⁷R⁸, —CN, C₁-C₆ alkyl, and C₁-C₆ alkoxy.

83. The compounds of any one of items 59-81, wherein Ar_{T} is selected from pyridinyl, pyrimidinyl, pyrazinyl, thiophenyl, pyrazolyl and imidazolyl, wherein Ar_{T} is optionally substituted by one or more substituents independently selected from halogen, —OH, —NR⁷R⁸, —CN, C₁-C₆ alkyl, and C₁-C₆ alkoxy.

84. The compounds of any one of items 59-81 or 83, wherein Ar_{T} is selected from thiophenyl, pyrazolyl, and imidazolyl, wherein Ar_{T} is optionally substituted by one or more substituents independently selected from halogen, —OH, —NR⁷R⁸, —CN, C₁-C₆alkyl, and C₁-C₆alkoxy.

85. The compounds of any one of items 59-81 or 83, wherein Ar_{T} is imidazolyl optionally substituted by methyl.

86. The compounds of any one of items 59-85, wherein R_{L} is —C(═O)OR⁹.

87. The compound of item 86, wherein R⁹ is selected from

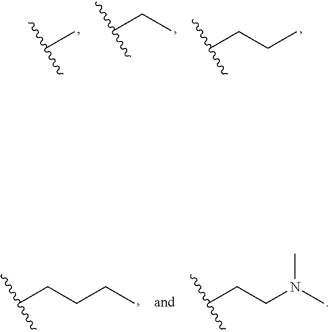

88. The compound of any one of items 59-85, wherein R_{L} is —C(═O)NR¹⁰R¹¹; R¹⁰ is hydrogen; and R¹¹ is selected from hydrogen,

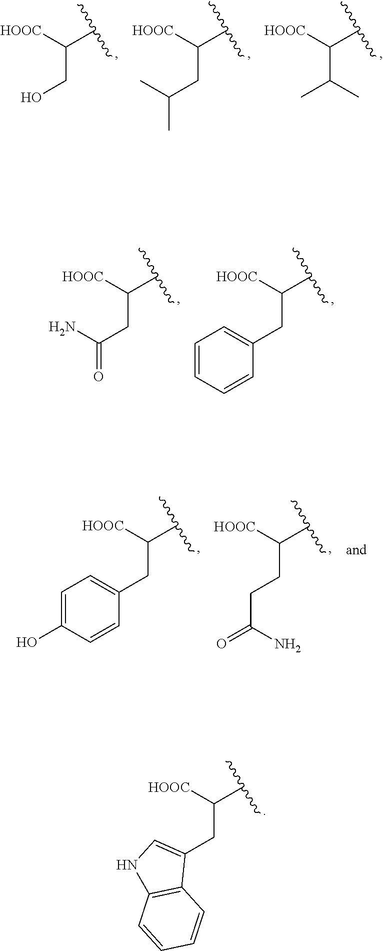

89. The compound of any one of items 59-85, wherein R_{L} is —NHC(═O)R¹² and R¹² is methyl.

90. The compound of any one of items 59-85, wherein R_{L} is —NHS(═O)₂R¹² and R¹² is selected from phenyl, toluyl, and methyl.

91. The compound of any one of items 59-85, wherein R_{L} is —C(═O)NHS(═O)R¹².

92. The compound of item 91, wherein R¹² is selected from methyl, butyl, and phenyl.

93. The compound of any one of items 59-85, wherein R_{L} is —C(═O)OH.

94. The compound of any one of items 59-85, wherein R_{L} is tetrazolyl.

95. The compound of item 59-85, wherein R_{L} is triazolyl, optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹R², —C(═O)R¹², aryl, and C₁-C₆ alkyl-(aryl).

96. The compound of any one of items 59-85, wherein R_{L} is triazolyl.

97. The compound of any one of items 59-96, wherein each R¹ and R² is independently selected from hydrogen and C₁-C₆ alkyl, or R¹ and R² are taken together with the N to which they are attached to form an optionally substituted C₂-C₈ heterocycloalkyl optionally substituted with one or more C₁-C₆ alkyl substituents.

98. The compound of any one of items 59-97, wherein each R¹, R², R⁷ and R⁸ is hydrogen.

99. The compound of any one of items 59-98, wherein the compound or a pharmaceutically acceptable salt thereof is in the form of a prodrug.

100. The compound of item 99, wherein the prodrug comprises an ester moiety.

101. The compound of item 99, wherein the prodrug comprises an amide moiety.

102. The compound of item 1, wherein the compound of Formula (I) is selected from:

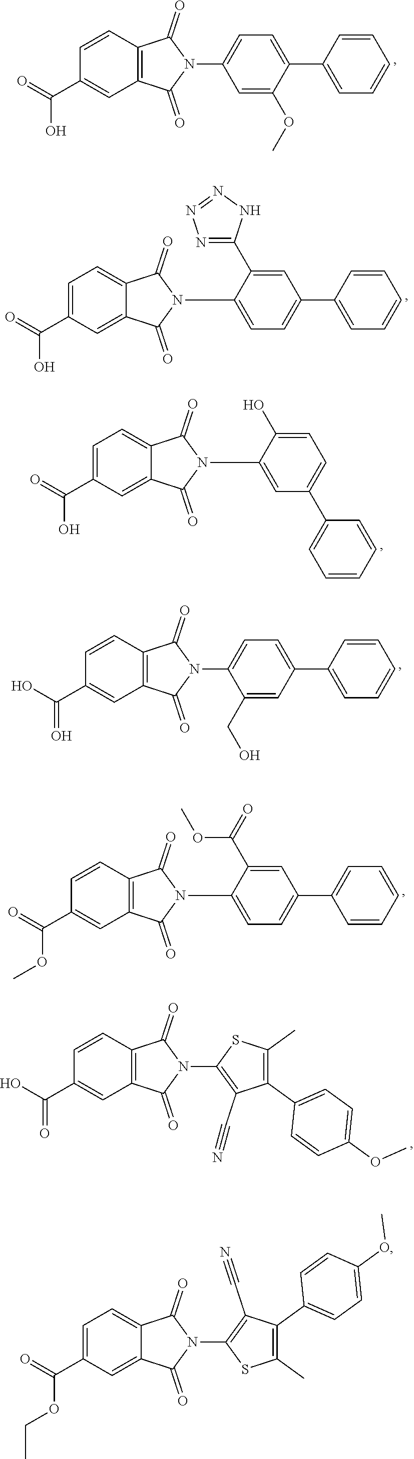

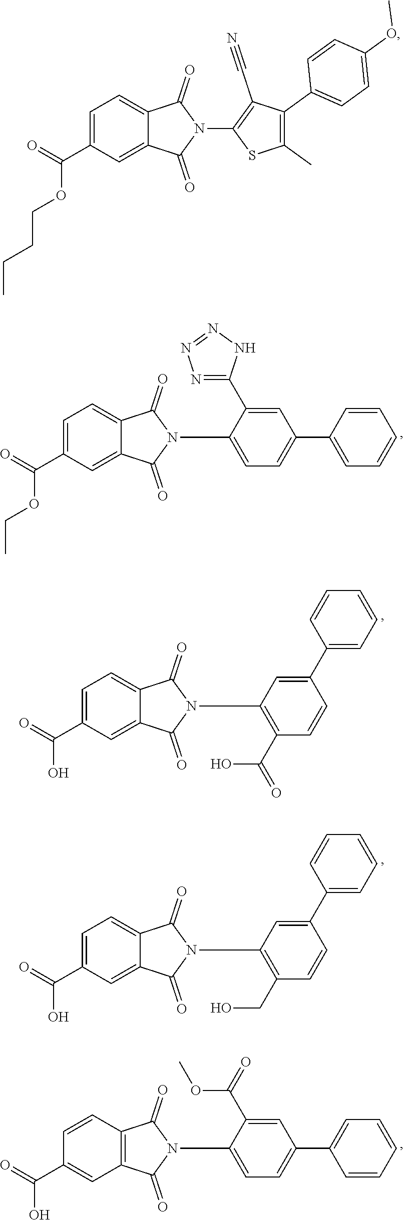 [SMILES: CCCCOC(=O)c1ccc2c(c1)C(=O)N(c1sc(C)c(-c3ccc(OC)cc3)c1C#N)C2=O ; CCOC(=O)c1ccc2c(c1)C(=O)N(c1ccc(-c3ccccc3)cc1-c1nnn[nH]1)C2=O ; O=C(O)c1ccc2c(c1)C(=O)N(c1cc(-c3ccccc3)ccc1C(=O)O)C2=O ; O=C(O)c1ccc2c(c1)C(=O)N(c1cc(-c3ccccc3)ccc1CO)C2=O ; COC(=O)c1cc(-c2ccccc2)ccc1N1C(=O)c2ccc(C(=O)O)cc2C1=O]

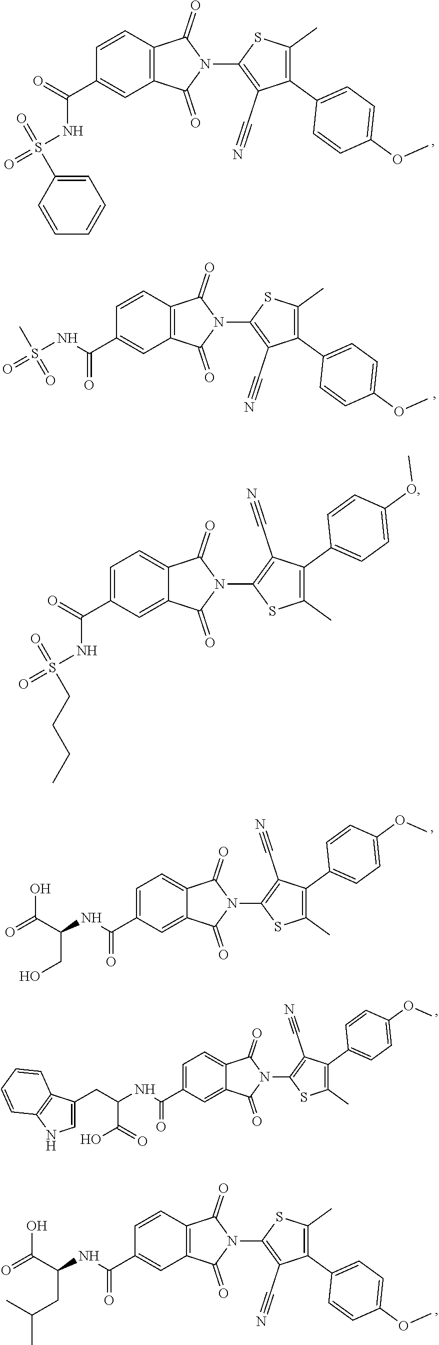 [SMILES: COc1ccc(-c2c(C)sc(N3C(=O)c4ccc(C(=O)NS(=O)(=O)c5ccccc5)cc4C3=O)c2C#N)cc1 ; COc1ccc(-c2c(C)sc(N3C(=O)c4ccc(C(=O)NS(C)(=O)=O)cc4C3=O)c2C#N)cc1 ; CCCCS(=O)(=O)NC(=O)c1ccc2c(c1)C(=O)N(c1sc(C)c(-c3ccc(OC)cc3)c1C#N)C2=O ; COc1ccc(-c2c(C)sc(N3C(=O)c4ccc(C(=O)N[C@@H](CO)C(=O)O)cc4C3=O)c2C#N)cc1 ; COc1ccc(-c2c(C)sc(N3C(=O)c4ccc(C(=O)NC(Cc5c[nH]c6ccccc56)C(=O)O)cc4C3=O)c2C#N)cc1 ; COc1ccc(-c2c(C)sc(N3C(=O)c4ccc(C(=O)N[C@@H](CC(C)C)C(=O)O)cc4C3=O)c2C#N)cc1]

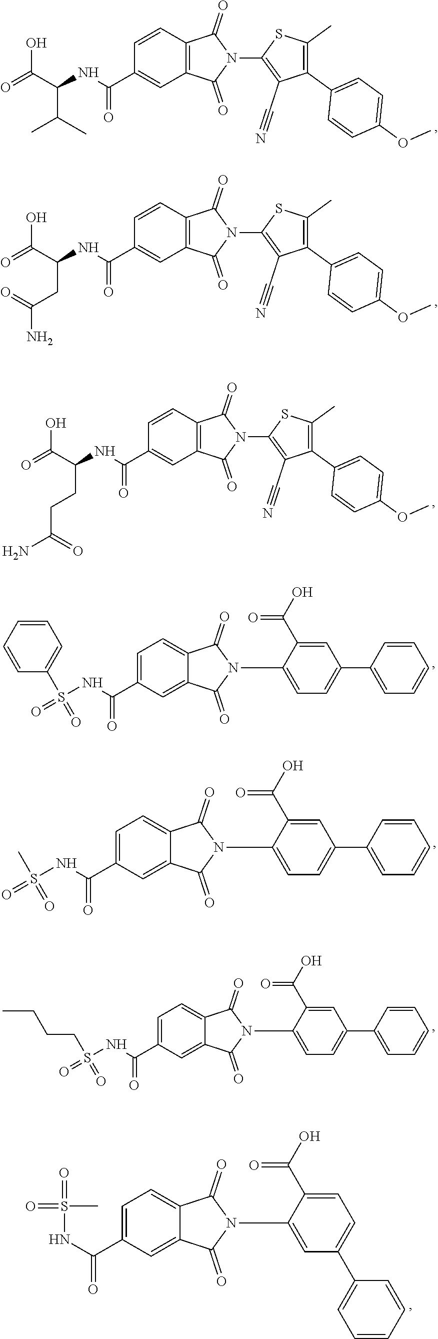

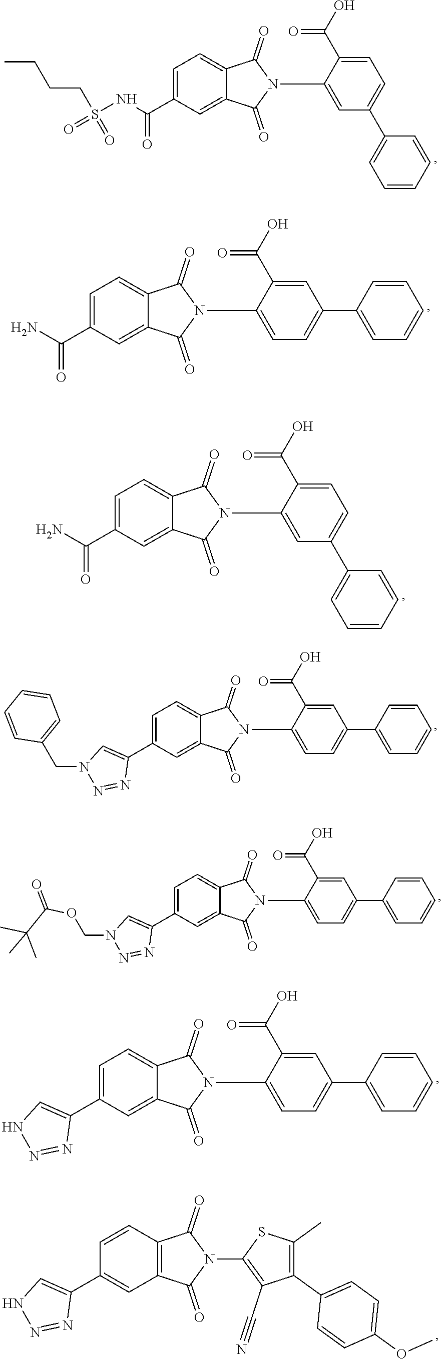

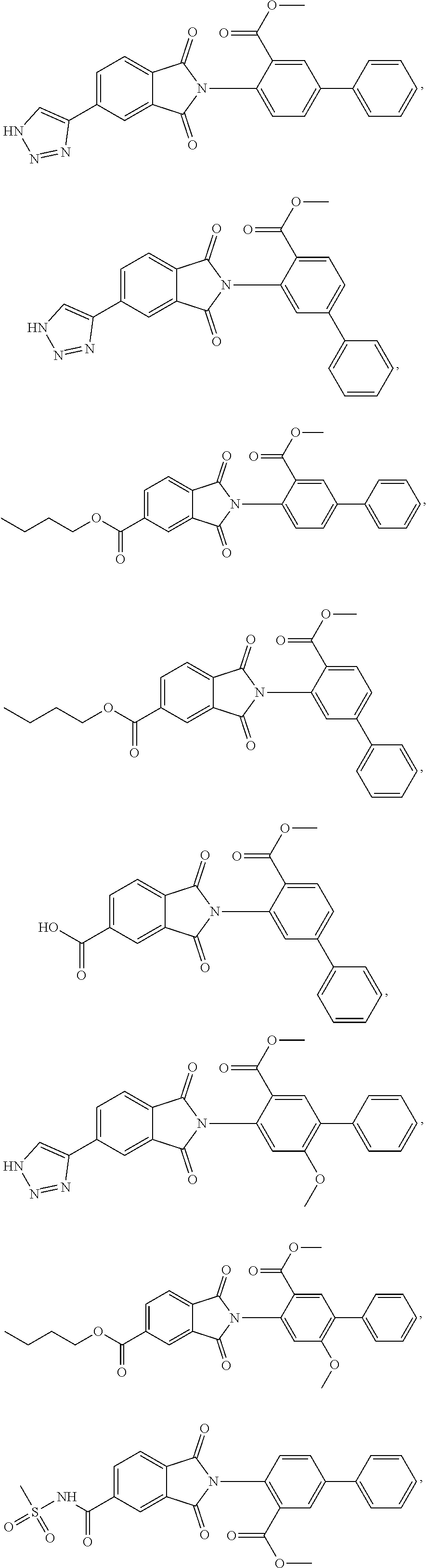

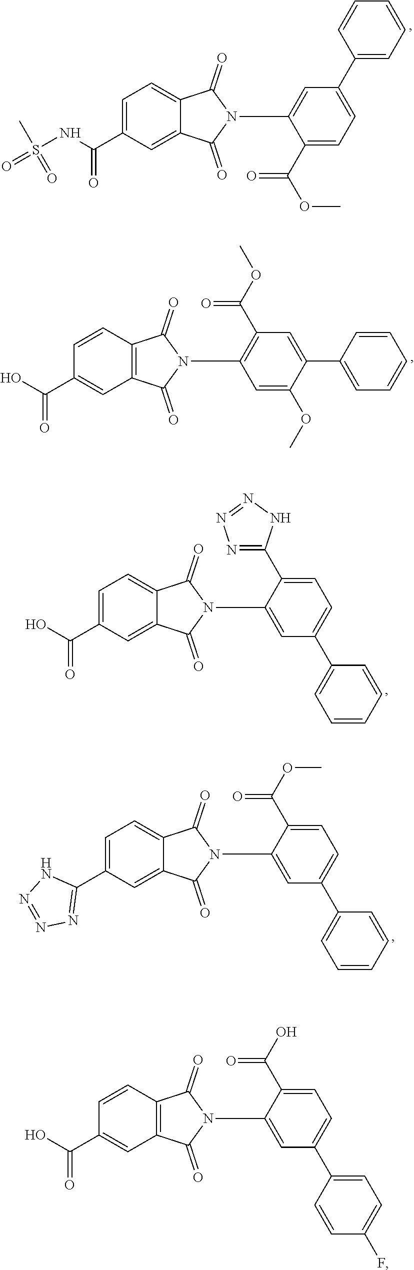

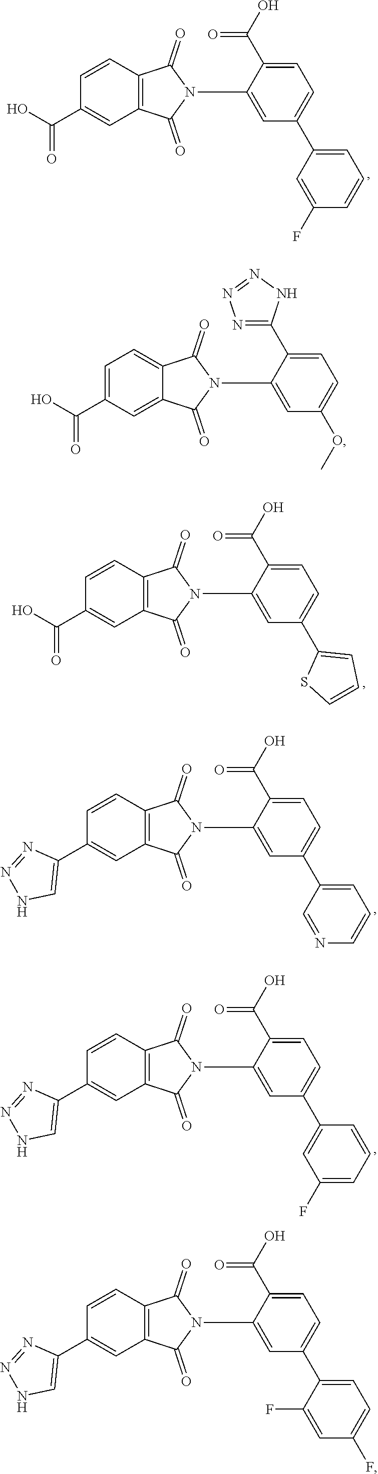

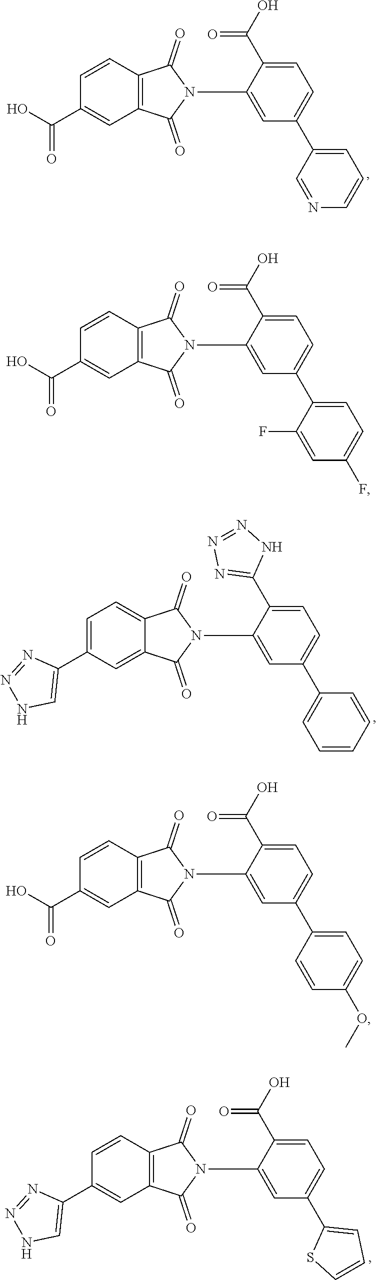 [SMILES: O=C(O)c1ccc2c(c1)C(=O)N(c1cc(-c3cccnc3)ccc1C(=O)O)C2=O ; O=C(O)c1ccc2c(c1)C(=O)N(c1cc(-c3ccc(F)cc3F)ccc1C(=O)O)C2=O ; O=C1c2ccc(-c3c[nH]nn3)cc2C(=O)N1c1cc(-c2ccccc2)ccc1-c1nnn[nH]1 ; COc1ccc(-c2ccc(C(=O)O)c(N3C(=O)c4ccc(C(=O)O)cc4C3=O)c2)cc1 ; O=C(O)c1ccc(-c2cccs2)cc1N1C(=O)c2ccc(-c3c[nH]nn3)cc2C1=O]

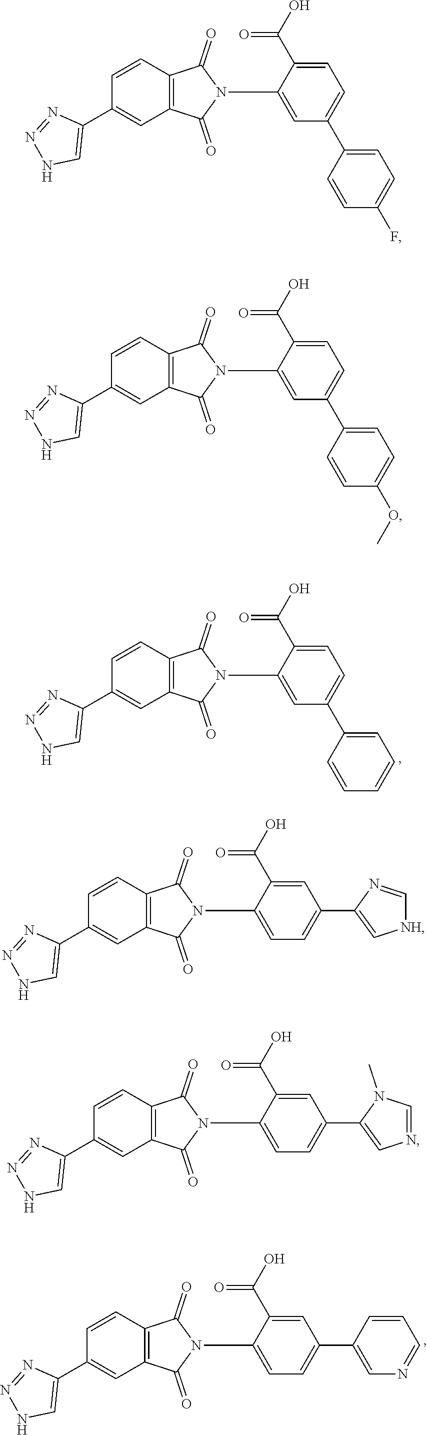 [SMILES: O=C(O)c1ccc(-c2ccc(F)cc2)cc1N1C(=O)c2ccc(-c3c[nH]nn3)cc2C1=O ; COc1ccc(-c2ccc(C(=O)O)c(N3C(=O)c4ccc(-c5c[nH]nn5)cc4C3=O)c2)cc1 ; O=C(O)c1ccc(-c2ccccc2)cc1N1C(=O)c2ccc(-c3c[nH]nn3)cc2C1=O ; O=C(O)c1cc(-c2c[nH]cn2)ccc1N1C(=O)c2ccc(-c3c[nH]nn3)cc2C1=O ; Cn1cncc1-c1ccc(N2C(=O)c3ccc(-c4c[nH]nn4)cc3C2=O)c(C(=O)O)c1 ; O=C(O)c1cc(-c2cccnc2)ccc1N1C(=O)c2ccc(-c3c[nH]nn3)cc2C1=O]

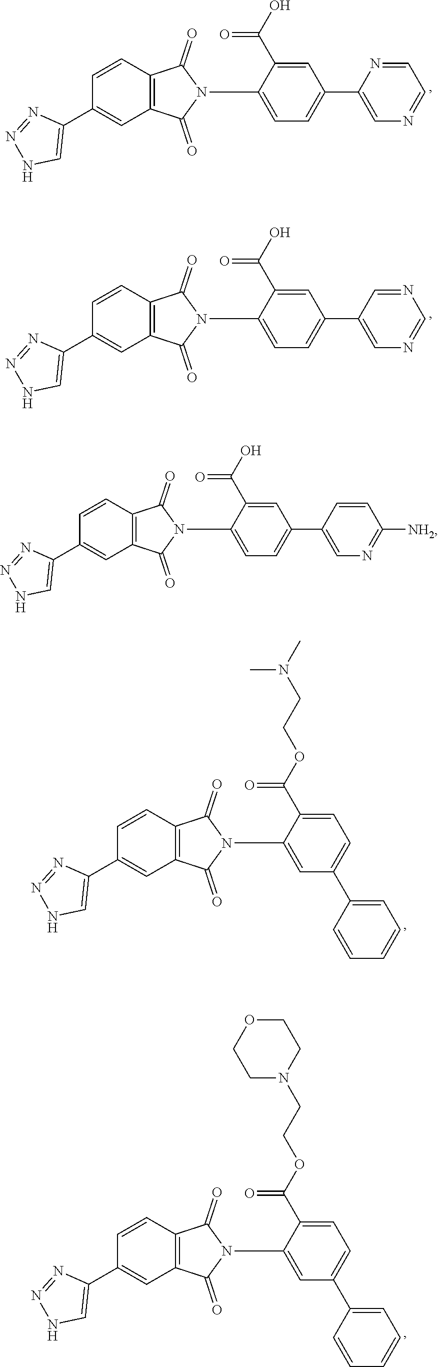 [SMILES: O=C(O)c1cc(-c2cnccn2)ccc1N1C(=O)c2ccc(-c3c[nH]nn3)cc2C1=O ; O=C(O)c1cc(-c2cncnc2)ccc1N1C(=O)c2ccc(-c3c[nH]nn3)cc2C1=O ; Nc1ccc(-c2ccc(N3C(=O)c4ccc(-c5c[nH]nn5)cc4C3=O)c(C(=O)O)c2)cn1 ; CN(C)CCOC(=O)c1ccc(-c2ccccc2)cc1N1C(=O)c2ccc(-c3c[nH]nn3)cc2C1=O ; O=C(OCCN1CCOCC1)c1ccc(-c2ccccc2)cc1N1C(=O)c2ccc(-c3c[nH]nn3)cc2C1=O]

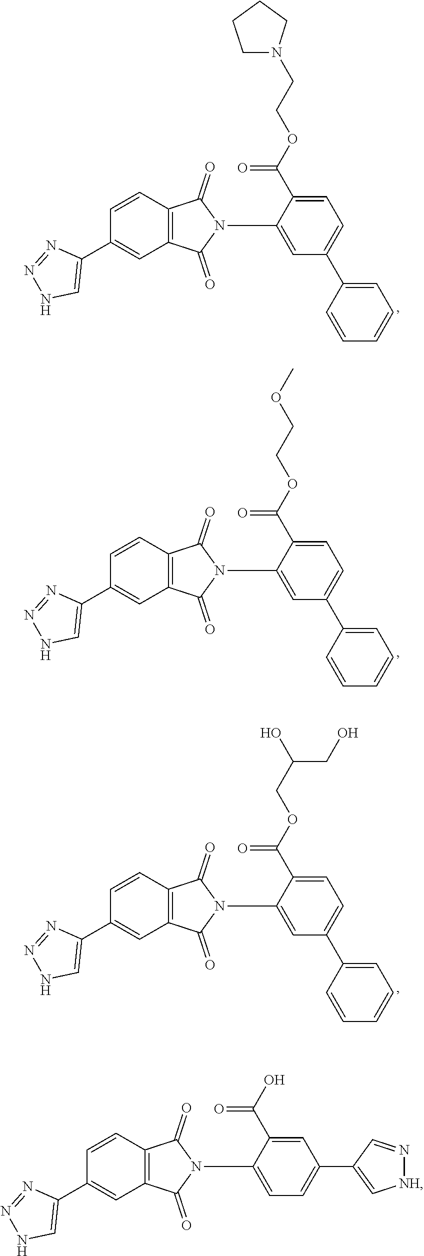

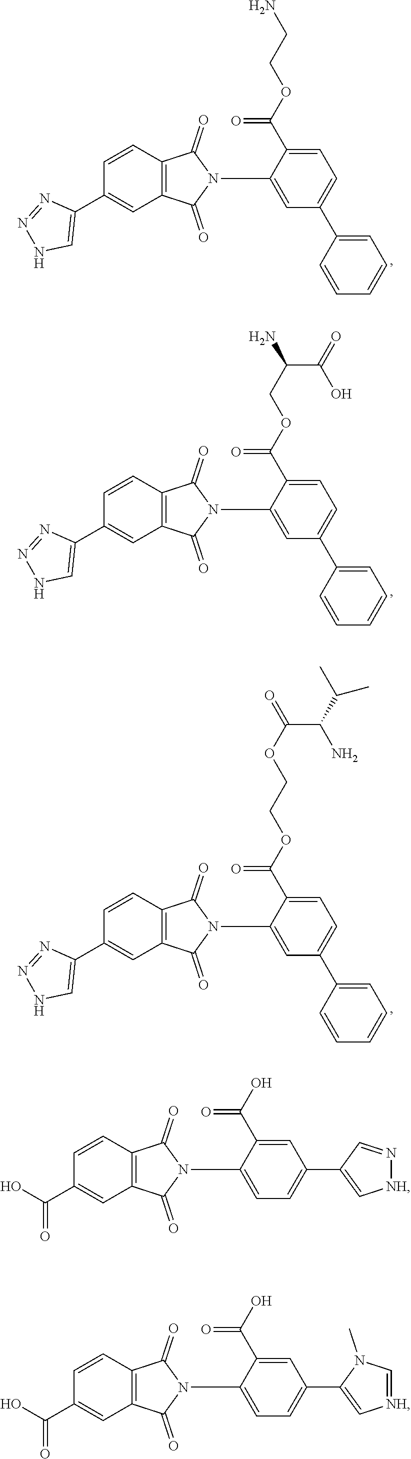

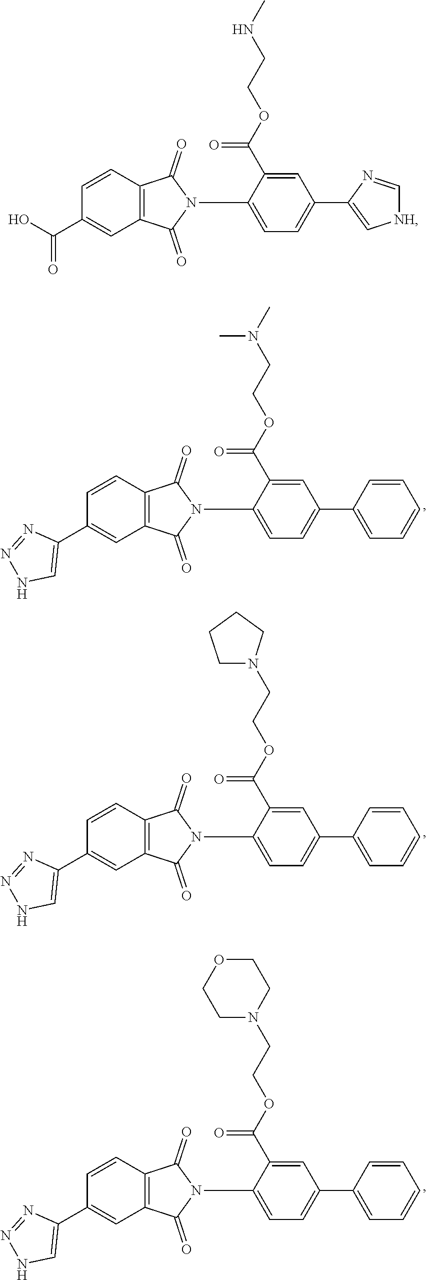 [SMILES: CNCCOC(=O)c1cc(-c2c[nH]cn2)ccc1N1C(=O)c2ccc(C(=O)O)cc2C1=O ; CN(C)CCOC(=O)c1cc(-c2ccccc2)ccc1N1C(=O)c2ccc(-c3c[nH]nn3)cc2C1=O ; O=C(OCCN1CCCC1)c1cc(-c2ccccc2)ccc1N1C(=O)c2ccc(-c3c[nH]nn3)cc2C1=O ; O=C(OCCN1CCOCC1)c1cc(-c2ccccc2)ccc1N1C(=O)c2ccc(-c3c[nH]nn3)cc2C1=O]

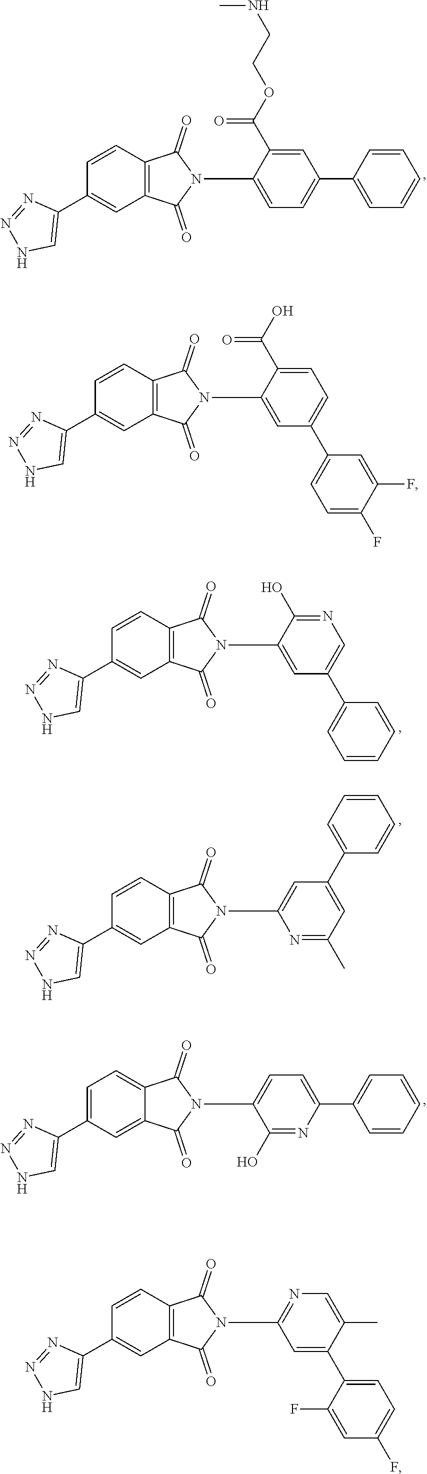

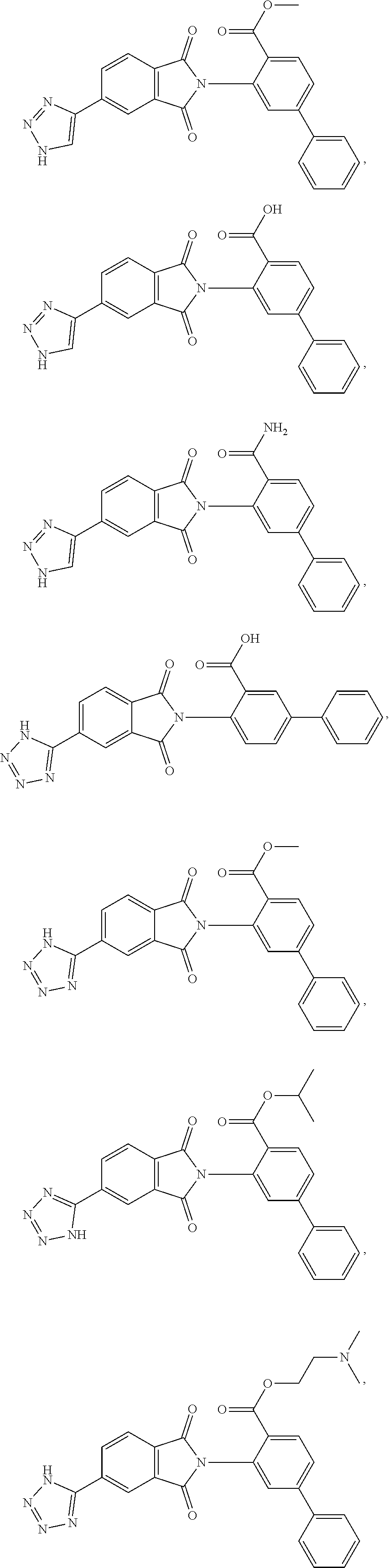

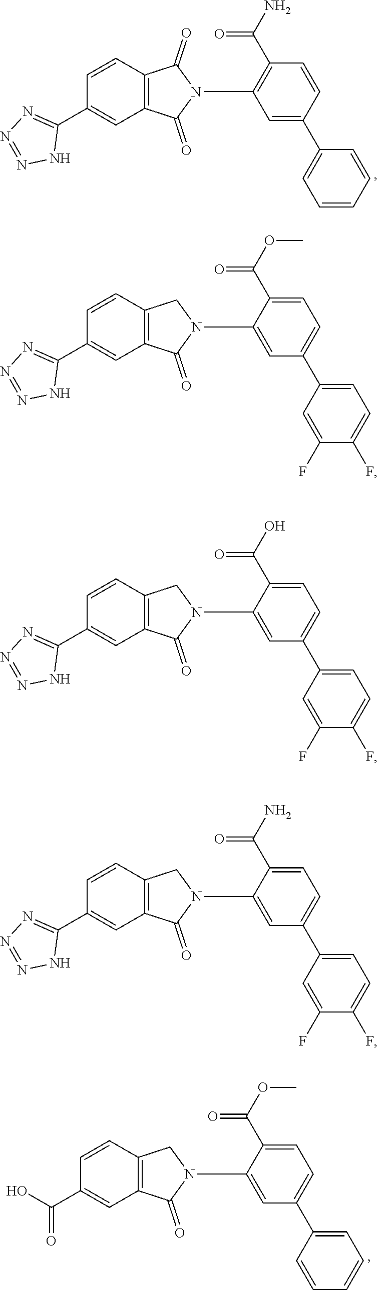

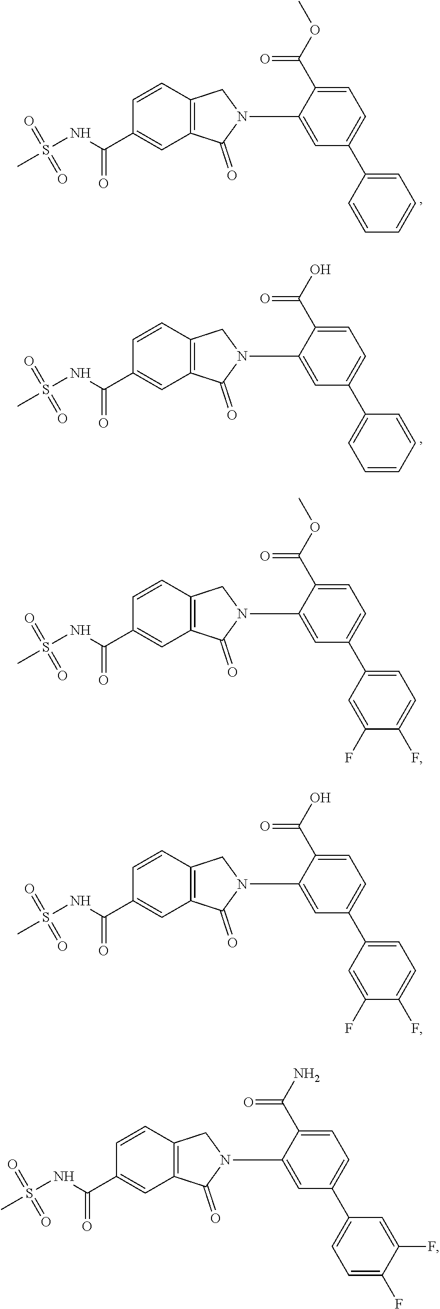 [SMILES: COC(=O)c1ccc(-c2ccccc2)cc1N1Cc2ccc(C(=O)NS(C)(=O)=O)cc2C1=O ; CS(=O)(=O)NC(=O)c1ccc2c(c1)C(=O)N(c1cc(-c3ccccc3)ccc1C(=O)O)C2 ; COC(=O)c1ccc(-c2ccc(F)c(F)c2)cc1N1Cc2ccc(C(=O)NS(C)(=O)=O)cc2C1=O ; CS(=O)(=O)NC(=O)c1ccc2c(c1)C(=O)N(c1cc(-c3ccc(F)c(F)c3)ccc1C(=O)O)C2 ; CS(=O)(=O)NC(=O)c1ccc2c(c1)C(=O)N(c1cc(-c3ccc(F)c(F)c3)ccc1C(N)=O)C2]

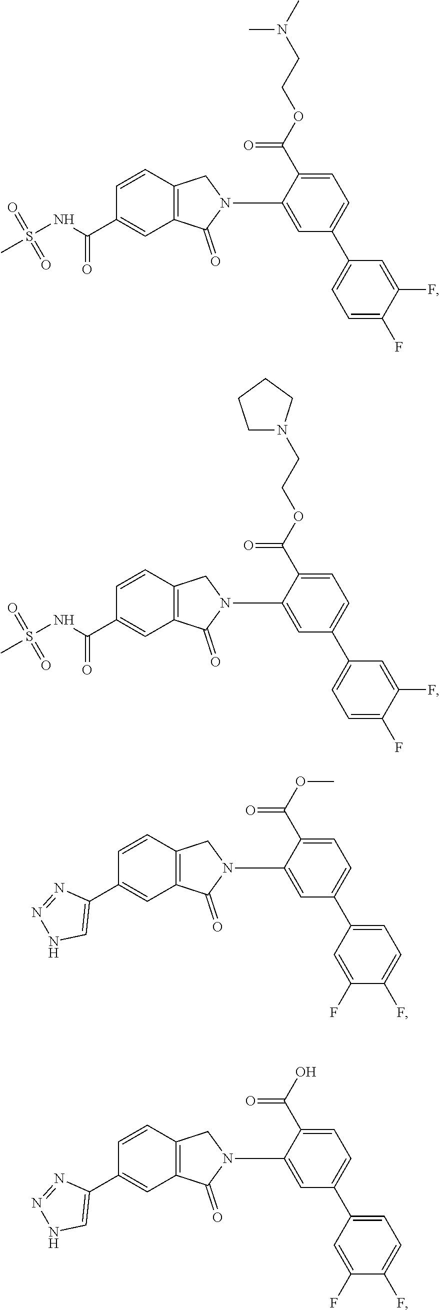 [SMILES: CN(C)CCOC(=O)c1ccc(-c2ccc(F)c(F)c2)cc1N1Cc2ccc(C(=O)NS(C)(=O)=O)cc2C1=O ; CS(=O)(=O)NC(=O)c1ccc2c(c1)C(=O)N(c1cc(-c3ccc(F)c(F)c3)ccc1C(=O)OCCN1CCCC1)C2 ; COC(=O)c1ccc(-c2ccc(F)c(F)c2)cc1N1Cc2ccc(-c3c[nH]nn3)cc2C1=O ; O=C(O)c1ccc(-c2ccc(F)c(F)c2)cc1N1Cc2ccc(-c3c[nH]nn3)cc2C1=O]

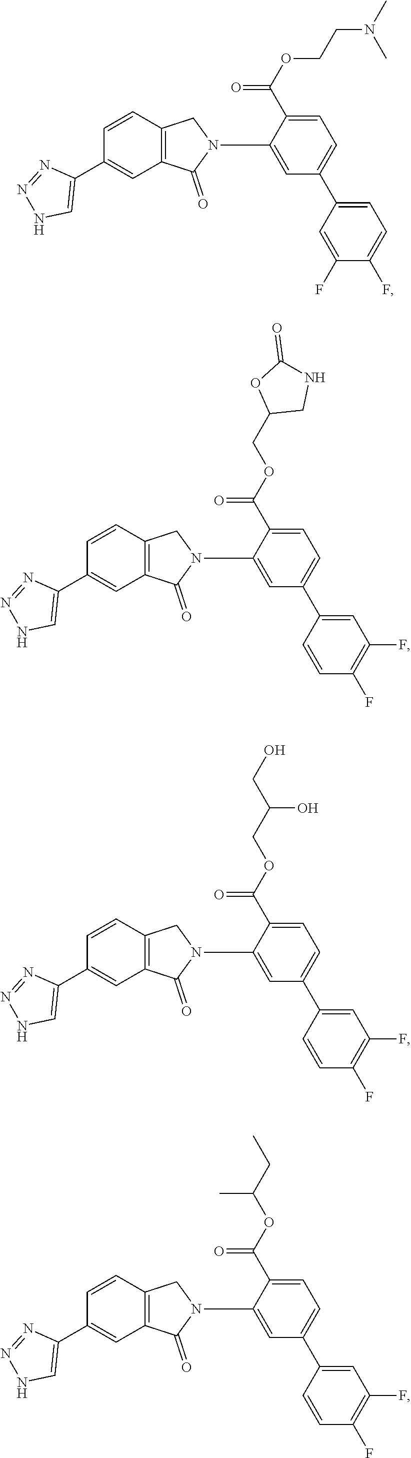 [SMILES: CN(C)CCOC(=O)c1ccc(-c2ccc(F)c(F)c2)cc1N1Cc2ccc(-c3c[nH]nn3)cc2C1=O ; O=C1NCC(COC(=O)c2ccc(-c3ccc(F)c(F)c3)cc2N2Cc3ccc(-c4c[nH]nn4)cc3C2=O)O1 ; O=C(OCC(O)CO)c1ccc(-c2ccc(F)c(F)c2)cc1N1Cc2ccc(-c3c[nH]nn3)cc2C1=O ; CCC(C)OC(=O)c1ccc(-c2ccc(F)c(F)c2)cc1N1Cc2ccc(-c3c[nH]nn3)cc2C1=O]

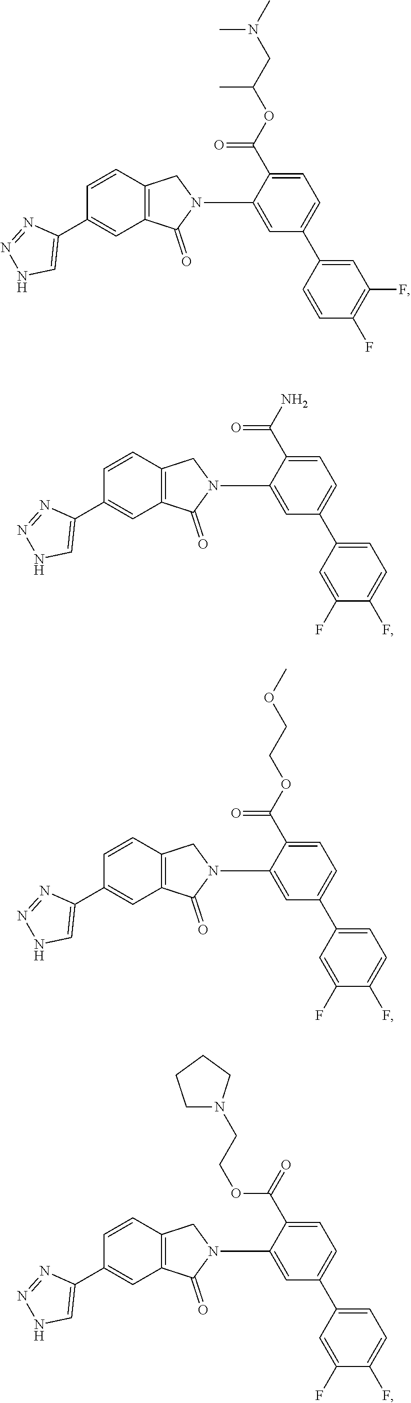 [SMILES: CC(CN(C)C)OC(=O)c1ccc(-c2ccc(F)c(F)c2)cc1N1Cc2ccc(-c3c[nH]nn3)cc2C1=O ; NC(=O)c1ccc(-c2ccc(F)c(F)c2)cc1N1Cc2ccc(-c3c[nH]nn3)cc2C1=O ; COCCOC(=O)c1ccc(-c2ccc(F)c(F)c2)cc1N1Cc2ccc(-c3c[nH]nn3)cc2C1=O ; O=C(OCCN1CCCC1)c1ccc(-c2ccc(F)c(F)c2)cc1N1Cc2ccc(-c3c[nH]nn3)cc2C1=O]

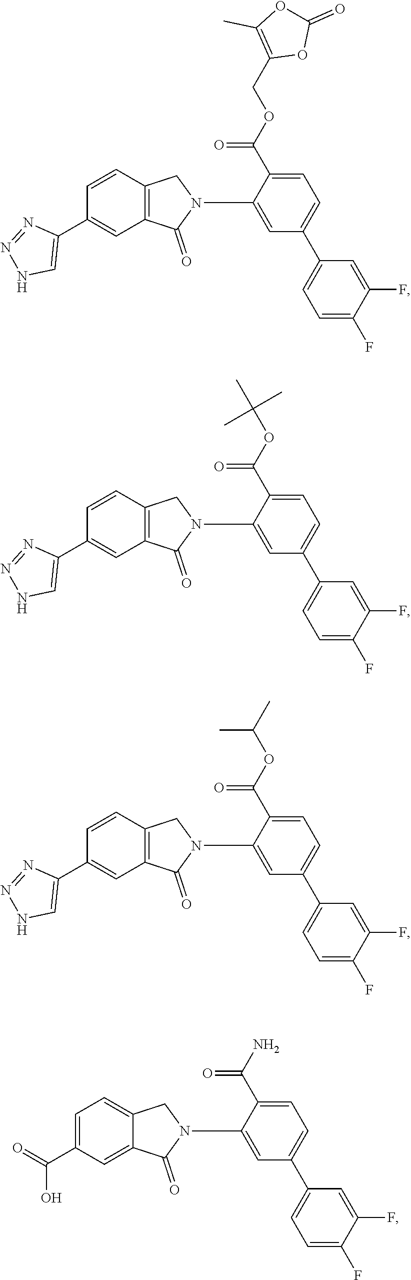 [SMILES: Cc1oc(=O)oc1COC(=O)c1ccc(-c2ccc(F)c(F)c2)cc1N1Cc2ccc(-c3c[nH]nn3)cc2C1=O ; CC(C)(C)OC(=O)c1ccc(-c2ccc(F)c(F)c2)cc1N1Cc2ccc(-c3c[nH]nn3)cc2C1=O ; CC(C)OC(=O)c1ccc(-c2ccc(F)c(F)c2)cc1N1Cc2ccc(-c3c[nH]nn3)cc2C1=O ; NC(=O)c1ccc(-c2ccc(F)c(F)c2)cc1N1Cc2ccc(C(=O)O)cc2C1=O]

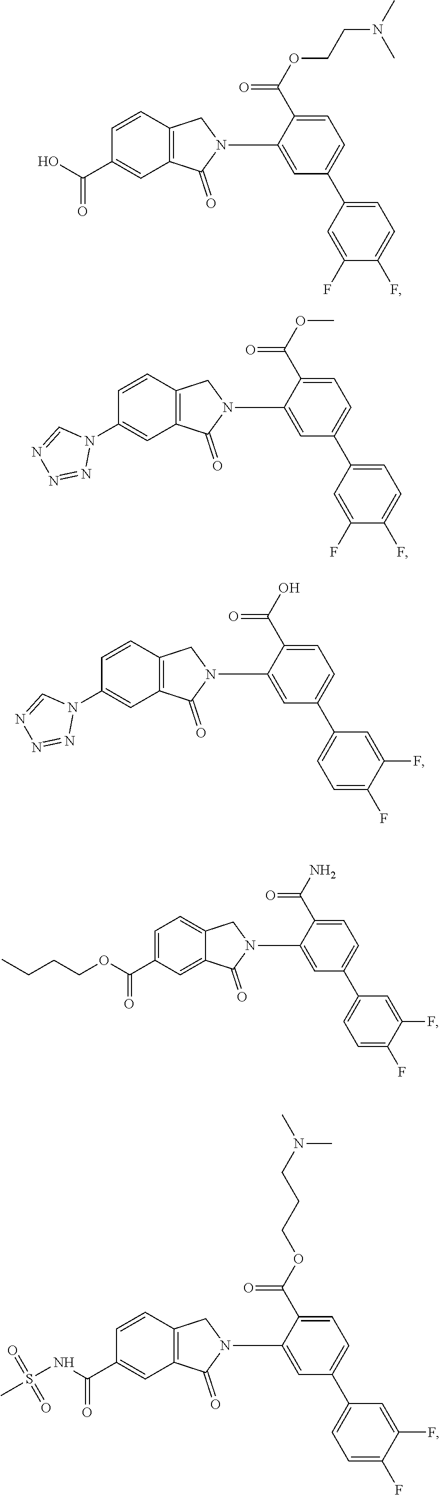

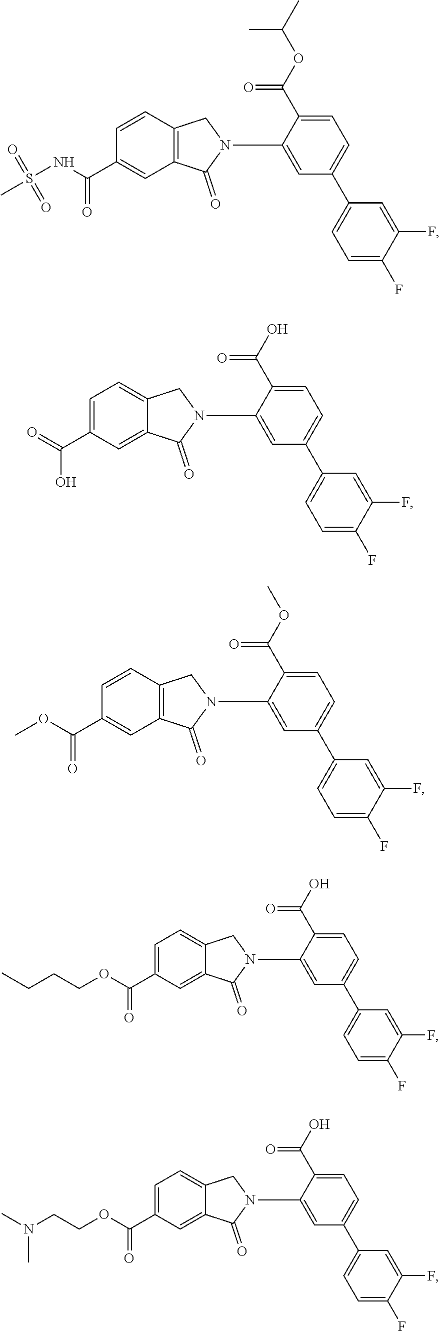 [SMILES: CC(C)OC(=O)c1ccc(-c2ccc(F)c(F)c2)cc1N1Cc2ccc(C(=O)NS(C)(=O)=O)cc2C1=O ; O=C(O)c1ccc2c(c1)C(=O)N(c1cc(-c3ccc(F)c(F)c3)ccc1C(=O)O)C2 ; COC(=O)c1ccc2c(c1)C(=O)N(c1cc(-c3ccc(F)c(F)c3)ccc1C(=O)OC)C2 ; CCCCOC(=O)c1ccc2c(c1)C(=O)N(c1cc(-c3ccc(F)c(F)c3)ccc1C(=O)O)C2 ; CN(C)CCOC(=O)c1ccc2c(c1)C(=O)N(c1cc(-c3ccc(F)c(F)c3)ccc1C(=O)O)C2]

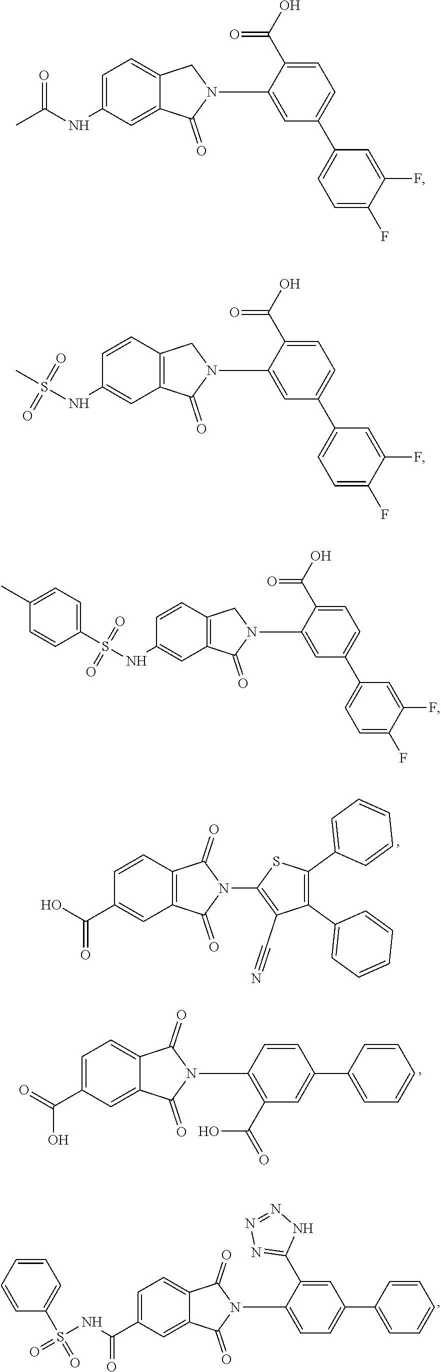 [SMILES: CC(=O)Nc1ccc2c(c1)C(=O)N(c1cc(-c3ccc(F)c(F)c3)ccc1C(=O)O)C2 ; CS(=O)(=O)Nc1ccc2c(c1)C(=O)N(c1cc(-c3ccc(F)c(F)c3)ccc1C(=O)O)C2 ; Cc1ccc(S(=O)(=O)Nc2ccc3c(c2)C(=O)N(c2cc(-c4ccc(F)c(F)c4)ccc2C(=O)O)C3)cc1 ; N#Cc1c(N2C(=O)c3ccc(C(=O)O)cc3C2=O)sc(-c2ccccc2)c1-c1ccccc1 ; O=C(O)c1ccc2c(c1)C(=O)N(c1ccc(-c3ccccc3)cc1C(=O)O)C2=O ; O=C(NS(=O)(=O)c1ccccc1)c1ccc2c(c1)C(=O)N(c1ccc(-c3ccccc3)cc1-c1nnn[nH]1)C2=O]

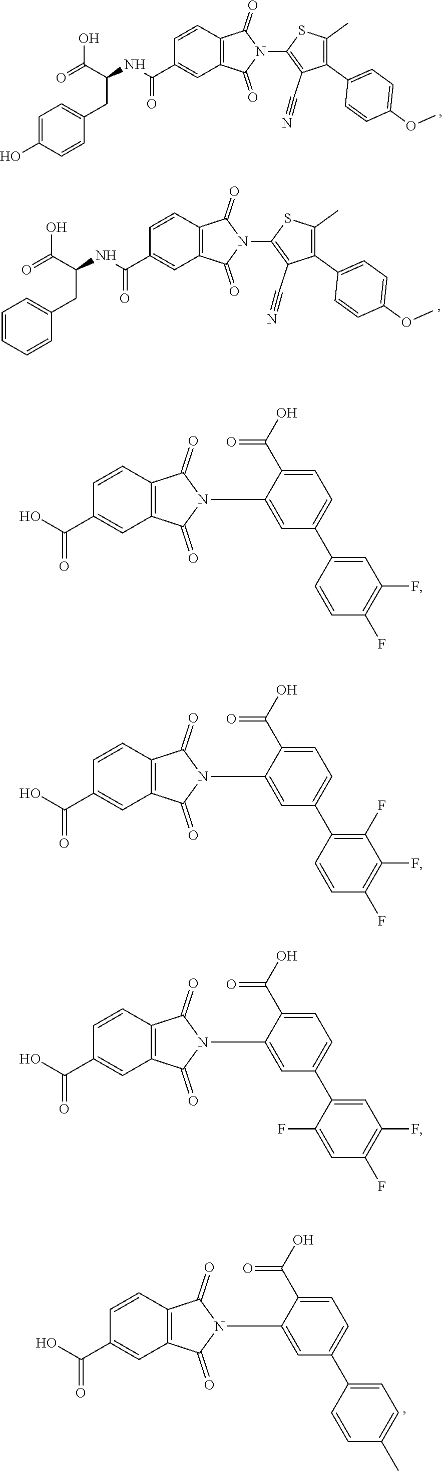 [SMILES: COc1ccc(-c2c(C)sc(N3C(=O)c4ccc(C(=O)N[C@@H](Cc5ccc(O)cc5)C(=O)O)cc4C3=O)c2C#N)cc1 ; COc1ccc(-c2c(C)sc(N3C(=O)c4ccc(C(=O)N[C@@H](Cc5ccccc5)C(=O)O)cc4C3=O)c2C#N)cc1 ; O=C(O)c1ccc2c(c1)C(=O)N(c1cc(-c3ccc(F)c(F)c3)ccc1C(=O)O)C2=O ; O=C(O)c1ccc2c(c1)C(=O)N(c1cc(-c3ccc(F)c(F)c3F)ccc1C(=O)O)C2=O ; O=C(O)c1ccc2c(c1)C(=O)N(c1cc(-c3cc(F)c(F)cc3F)ccc1C(=O)O)C2=O ; Cc1ccc(-c2ccc(C(=O)O)c(N3C(=O)c4ccc(C(=O)O)cc4C3=O)c2)cc1]

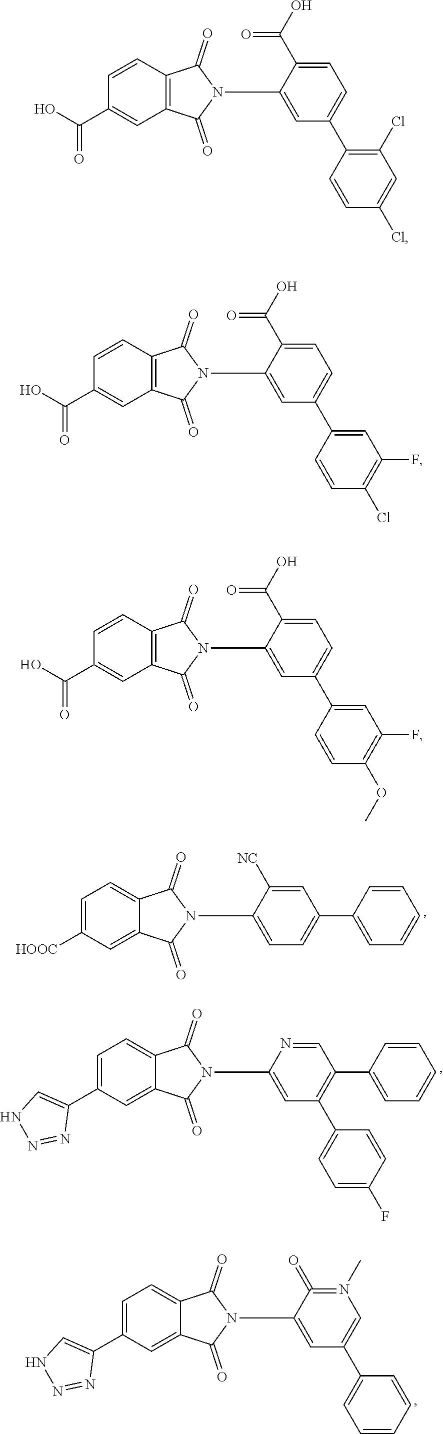

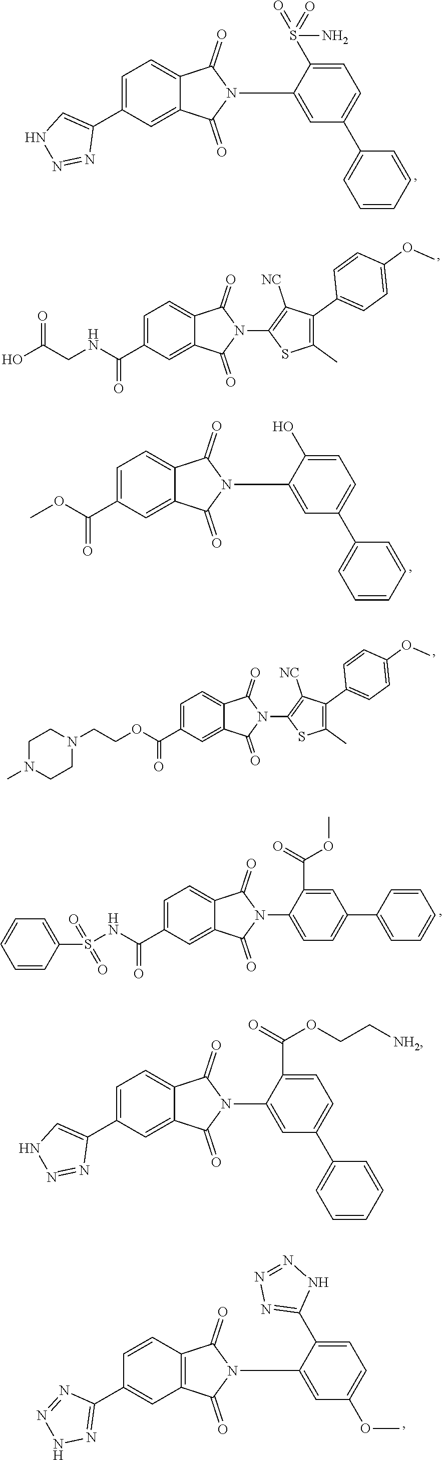

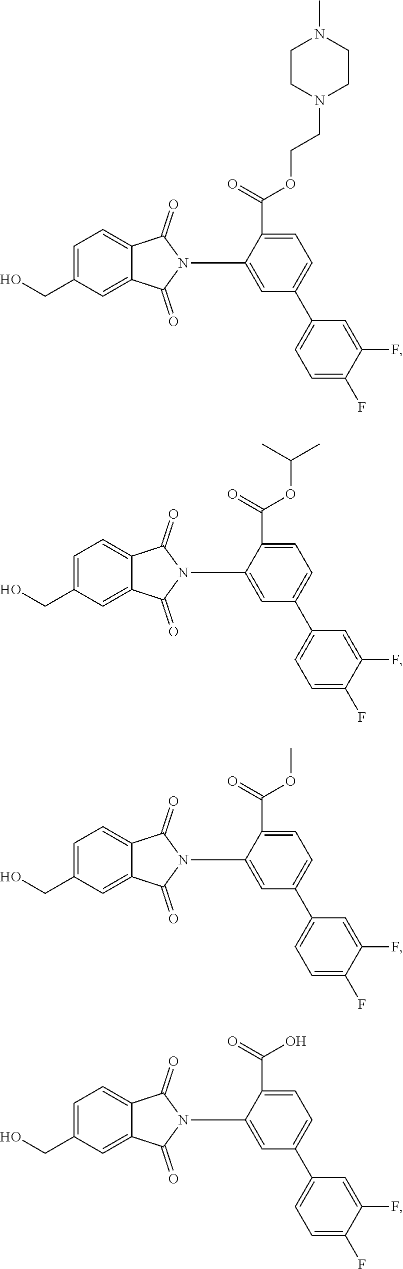 [SMILES: CN1CCN(CCOC(=O)c2ccc(-c3ccc(F)c(F)c3)cc2N2C(=O)c3ccc(CO)cc3C2=O)CC1 ; CC(C)OC(=O)c1ccc(-c2ccc(F)c(F)c2)cc1N1C(=O)c2ccc(CO)cc2C1=O ; COC(=O)c1ccc(-c2ccc(F)c(F)c2)cc1N1C(=O)c2ccc(CO)cc2C1=O ; O=C(O)c1ccc(-c2ccc(F)c(F)c2)cc1N1C(=O)c2ccc(CO)cc2C1=O]

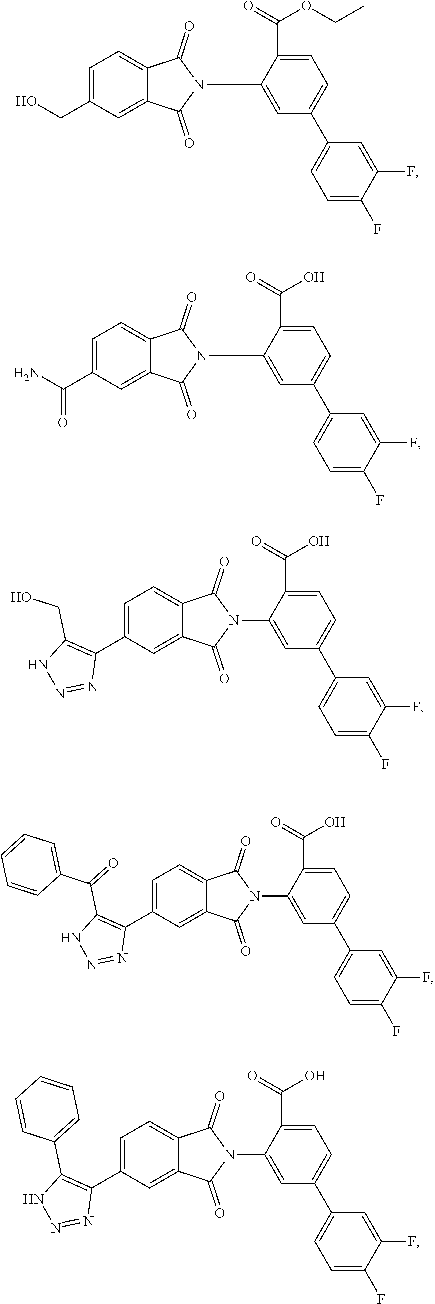 [SMILES: CCOC(=O)c1ccc(-c2ccc(F)c(F)c2)cc1N1C(=O)c2ccc(CO)cc2C1=O ; NC(=O)c1ccc2c(c1)C(=O)N(c1cc(-c3ccc(F)c(F)c3)ccc1C(=O)O)C2=O ; O=C(O)c1ccc(-c2ccc(F)c(F)c2)cc1N1C(=O)c2ccc(-c3nn[nH]c3CO)cc2C1=O ; O=C(O)c1ccc(-c2ccc(F)c(F)c2)cc1N1C(=O)c2ccc(-c3nn[nH]c3C(=O)c3ccccc3)cc2C1=O ; O=C(O)c1ccc(-c2ccc(F)c(F)c2)cc1N1C(=O)c2ccc(-c3nn[nH]c3-c3ccccc3)cc2C1=O]

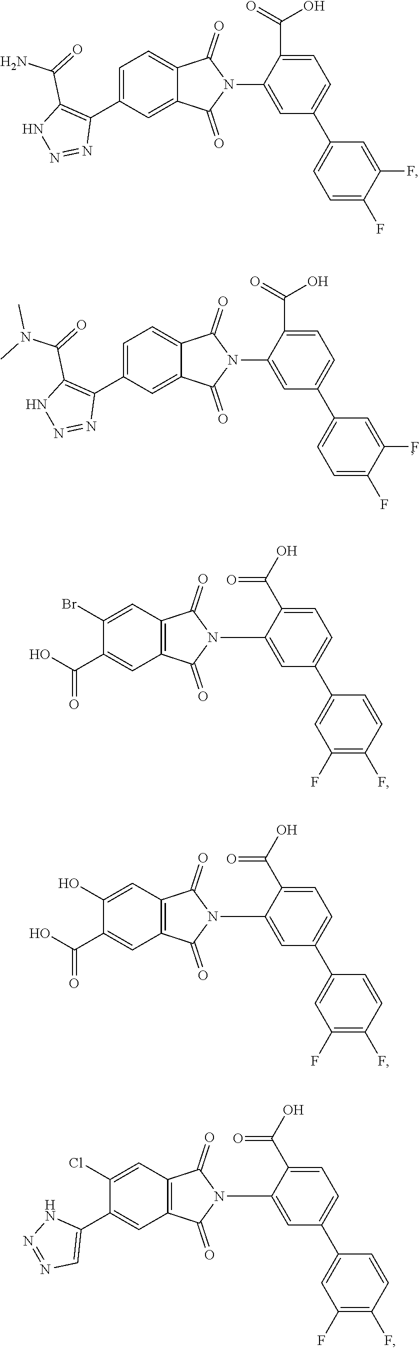 [SMILES: NC(=O)c1[nH]nnc1-c1ccc2c(c1)C(=O)N(c1cc(-c3ccc(F)c(F)c3)ccc1C(=O)O)C2=O ; CN(C)C(=O)c1[nH]nnc1-c1ccc2c(c1)C(=O)N(c1cc(-c3ccc(F)c(F)c3)ccc1C(=O)O)C2=O ; O=C(O)c1cc2c(cc1Br)C(=O)N(c1cc(-c3ccc(F)c(F)c3)ccc1C(=O)O)C2=O ; O=C(O)c1cc2c(cc1O)C(=O)N(c1cc(-c3ccc(F)c(F)c3)ccc1C(=O)O)C2=O ; O=C(O)c1ccc(-c2ccc(F)c(F)c2)cc1N1C(=O)c2cc(Cl)c(-c3cnn[nH]3)cc2C1=O]

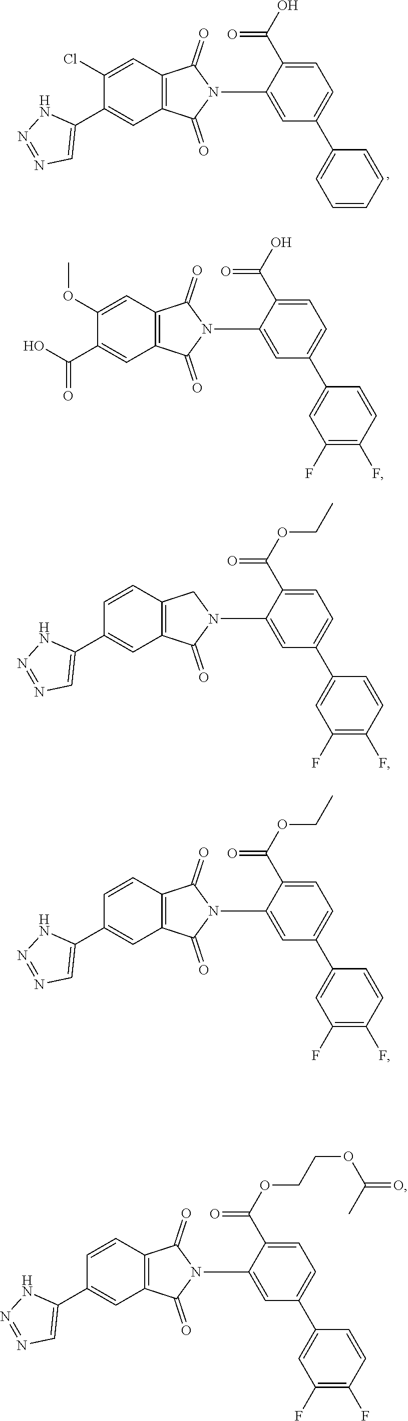

or a pharmaceutically acceptable salt thereof.

103. A compound selected from:

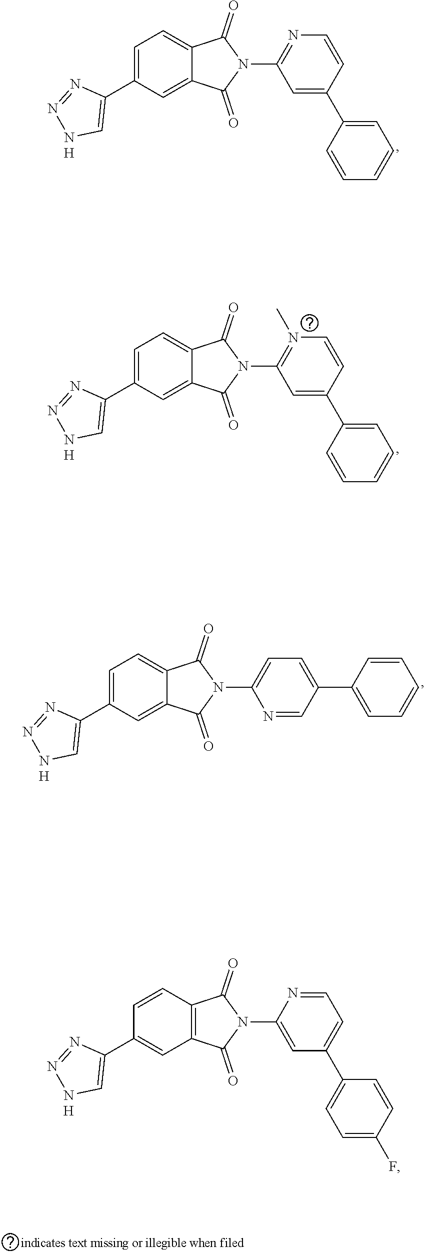

or a pharmaceutically acceptable salt thereof.

104. A compound of Formula (II):

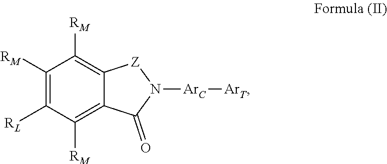

a prodrug thereof, a pharmaceutically acceptable salt thereof, or combination thereof, wherein:

Z is —C(═O)— or —C(R^{a})(R^{b})—;

R^{a} and R^{b} are each independently selected from hydrogen, halogen, —OH, C₁₁ alkyl, C₁₋₆ alkoxy;

Ar_{C} is selected from arylene and heteroarylene; wherein Ar_{C} is substituted with one or more R_{C};

each R_{C} is independently selected from halogen, —ON, optionally substituted C₁-C₆ alkyl, optionally substituted heteroaryl, optionally substituted aryl, —C(═O)OH, —C(═OR³, —C(═O)NR⁴R⁵, —S(═O)₂NR⁴R⁵, —NHS(═O)₂R⁶, and —C(═O)NHS(═O)₂R⁶;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, and C₂-C₈ heterocycloalkyl; and

wherein the aryl and heteroaryl are optionally substituted with one or more substituents independently selected from —OH, halogen, —C(═O)OR⁷, —C(═O)NR¹R², C₁-C₆ alkyl, C₁-C₆ alkoxy, and —NR⁷R⁸;

each R¹ and R² is independently selected from hydrogen and C₁-C₆ alkyl optionally substituted with one or more substituents independently selected from halogen, —C(═O)OR⁷, —C(═O)NR⁷R⁸, —OH, aryl, heteroaryl, C₃-C₈ cycloalkyl, and C₂-C₈ heterocycloalkyl;

or R¹ and R² are taken together with the N to which they are attached to form a C₂-C₈ heterocycloalkyl optionally substituted with one or more C₁-C₆ alkyl substituents;

each R³ is independently C₁-C₆ alkyl optionally substituted with one or more substituents independently selected from —OH, optionally substituted —OC(═O)C₁-C₆alkyl, optionally substituted —C(═O)OC₁-C₆ alkyl, C₁-C₆ alkoxy, —C(═O)OH, —NR¹R²;

wherein the —OC(═O)C₁-C₆ alkyl and —C(═O)OC₁-C₆ alkyl are optionally substituted with one or more substituents independently selected from with —OH or —NR⁷R⁸;

each R⁴ and R⁵ is independently selected from hydrogen and C₁-C₆ alkyl optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl;

or R⁴ and R⁵ are taken together with the N to which they are attached to form a C₂-C₈ heterocycloalkyl optionally substituted with one or more C₁-C₆ alkyl substituents;

each R⁶ are independently selected from optionally substituted C₁-C₆ alkyl and optionally substituted aryl;

wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and

wherein the aryl is optionally substituted with one or more substituents independently selected from —OH, halogen, —C(═O)OR⁷, —C(═O)NR⁷R⁸, C₁-C₆ alkyl, C₁-C₆alkoxy, and —NR⁷R⁸;

each R⁷ and R⁸ is independently selected from hydrogen and C₁-C₆ alkyl;

or R⁷ and R⁸ are taken together with the N to which they are attached to form an optionally substituted C₂-C₈ heterocycloalkyl optionally substituted with one or more C₁-C₆ alkyl substituents;

Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;

each R_{M} is independently selected from hydrogen, halogen, —OH, —CN, optionally substituted C₁-C₆alkyl, and optionally substituted C₁-C₆ alkoxy;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;

wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR⁷R⁸, —C(═O)R¹², aryl, or C₁-C₆ alkyl-(aryl);

R⁹ is C₁-C₆ alkyl optionally substituted with one or more substituents independently selected from —OH and —NR¹R²;

each R¹⁰ and R¹¹ is independently selected from hydrogen and C₁-C₆ alkyl optionally substituted with one or more substituents independently selected from —C(═O)OR⁷, —C(═O)NR¹R², —OH, aryl, hydroxyaryl or heteroaryl;

or R¹⁰ and R¹¹ are taken together with the N to which they are attached to form a C₂-C₈ heterocycloalkyl optionally substituted with one or more C₁-C₆ alkyl substituents;

R¹² is selected from C₁-C₆alkyl and aryl optionally substituted with one or more C₁-C₆alkyl substituents; and

wherein at least one R_{C} is —C(═O)OH; or R_{L} is —C(═O)OH.

105. The compound of item 104, wherein Z is —C(═O)—.

106. The compound of item 104, wherein Z is —C(R^{a})(R^{b})—, and R^{a} and R^{b} are each independently selected from hydrogen, fluorine and methyl.

107. The compound of item 104 or 106, wherein Z is —CH₂—.

108. The compound of any one of items 104-107, wherein each R_{M} is independently selected from hydrogen, halogen, —OH, —CN, C₁-C₆alkyl, and C₁-C₆ alkoxy.

109. The compound of any one of items 104-108, wherein one R_{M} is selected from hydrogen, halogen, —OH, —CN, C₁-C₆ alkyl, and C₁-C₆ alkoxy; and each other R_{M} is independently selected from hydrogen and halogen.

110. The compound of any one of items 104-109, wherein each R_{M} is hydrogen.

111. The compound of any one of items 104-110, wherein R_{L} is —C(═O)OH.

112. The compound of item 1, wherein the compound of Formula (I) is represented by Formula (III):

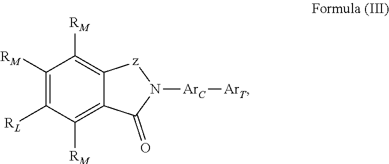

or a pharmaceutically acceptable salt thereof, wherein: Z is —C(═O)— or —C(R^{a})(R^{b})—;

R^{a} and R^{b} are each independently selected from hydrogen, halogen, —OH, C₁₋₆ alkyl, C₁₋₆ alkoxy;

Ar_{C} is selected from arylene and heteroarylene; wherein Ar_{C} is substituted with one or more R_{C};

each R_{C} is independently selected from halogen, —CN, optionally substituted C₁-C₆ alkyl, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;

wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, and C₂-C₈ heterocycloalkyl; and

wherein the aryl and heteroaryl are optionally substituted with one or more substituents independently selected from —OH, halogen, —C(═O)OR⁷, —C(═O)NR¹R², C₁-C₆ alkyl, C₁-C₆ alkoxy, and —NR⁷R⁸;

each R¹ and R² is independently selected from hydrogen and C₁-C₆ alkyl optionally substituted with one or more substituents independently selected from halogen, —C(═O)OR⁷, —C(═O)NR⁷R⁸, —OH, aryl, heteroaryl, C₃-C₈ cycloalkyl, and C₂-C₈ heterocycloalkyl;

or R¹ and R² are taken together with the N to which they are attached to form a C₂-C₈ heterocycloalkyl optionally substituted with one or more C₁-C₆ alkyl substituents;

each R³ is independently C₁-C₆ alkyl optionally substituted with one or more substituents independently selected from —OH, optionally substituted —OC(═O)C₁-C₆alkyl, optionally substituted —C(═O)OC₁-C₆ alkyl, C₁-C₆ alkoxy, —C(═O)OH, —NR¹R²;

wherein the —OC(═O)C₁-C₆ alkyl and —C(═O)OC₁-C₆ alkyl are optionally substituted with one or more substituents independently selected from with —OH or —NR⁷R⁸;

each R⁴ and R⁵ is independently selected from hydrogen and C₁-C₆ alkyl optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl;

or R⁴ and R⁵ are taken together with the N to which they are attached to form a C₂-C₈ heterocycloalkyl optionally substituted with one or more C₁-C₆ alkyl substituents;

R⁶ is selected from optionally substituted C₁-C₆ alkyl and optionally substituted aryl;

wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and

wherein the aryl is optionally substituted with one or more substituents independently selected from —OH, halogen, —C(═O)OR⁷, —C(═O)NR⁷R⁸, C₁-C₆ alkyl, C₁-C₆ alkoxy, and —NR⁷R⁸;

each R⁷ and R⁸ is independently selected from hydrogen and C₁-C₆ alkyl;

or R⁷ and R⁸ are taken together with the N to which they are attached to form an optionally substituted C₂-C₈ heterocycloalkyl optionally substituted with one or more C₁-C₆ alkyl substituents;

Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;

each R_{M} is independently selected from hydrogen, halogen, —OH, —CN, optionally substituted C₁-C₆alkyl, and optionally substituted C₁-C₆ alkoxy;

wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₃-C₈ cycloalkyl, —O—C₃-C₈ cycloalkyl, C₂-C₈ heterocycloalkyl, and —O—C₂-C₈ heterocycloalkyl;

R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;

wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR⁷R⁸, —C(═O)R¹², aryl, or C₁-C₆ alkyl-(aryl);

R⁹ is C₁-C₆ alkyl optionally substituted with one or more substituents independently selected from —OH and —NR¹R²;

each R¹⁰ and R¹¹ is independently selected from hydrogen and C₁-C₆ alkyl optionally substituted with one or more substituents independently selected from —C(═O)OH, —C(═O)NR¹R², —OH, aryl, hydroxyaryl or heteroaryl;

or R¹⁰ and R¹¹ are taken together with the N to which they are attached to form a C₂-C₈ heterocycloalkyl optionally substituted with one or more C₁-C₆ alkyl substituents;

R¹² is selected from C₁-C₆alkyl and aryl optionally substituted with one or more C₁-C₆alkyl substituents; and

wherein at least one R_{C} is —C(═O)OR³ or R_{L} is —C(═O)OR⁹.

113. The compound of item 112, wherein Z is —C(═O)—.

114. The compound of item 112, wherein Z is —C(R^{a})(R^{b})—, wherein R^{a} and R^{b} are each independently selected from hydrogen, fluorine and methyl.

115. The compound of item 112 or 114, wherein Z is —CH₂—.

116. The compound of any one of items 112-115, wherein each R_{M} is independently selected from hydrogen, halogen, —OH, —CN, C₁-C₆alkyl, and C₁-C₆ alkoxy.

117. The compound of any one of items 112-116, wherein one R_{M} is selected from hydrogen, halogen, —OH, —CN, C₁-C₆ alkyl, and C₁-C₆ alkoxy; and each other R_{M} is independently selected from hydrogen and halogen.

118. The compound of any one of items 112-117, wherein each R_{M} is hydrogen.

119. The compound of item 1, wherein the compound of Formula (I) is represented by Formula (IV):

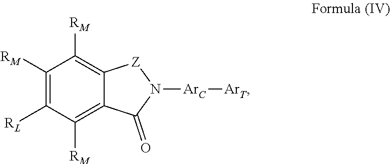

or a pharmaceutically acceptable salt thereof, wherein:

Z is —C(═O)— or —C(R^{a})(R^{b})—;

R^{a} and R^{b} are each independently selected from hydrogen, fluorine and methyl;

Ar_{C} is selected from arylene and heteroarylene; each substituted with one or more R_{C};

R_{C} is selected from —CN, —OH, C₁-C₆ alkoxy, C₁-C₆ alkyl, C₁-C₆ hydroxyalkyl, heteroaryl, aryl, —C(═O)OH, and —C(═O)OR³;

each R¹ and R² is independently selected from hydrogen and C₁-C₆ alkyl;

or R¹ and R² are taken together with the N to which they are attached to form a C₂-C₈ heterocycloalkyl;

each R³ is independently C₁-C₆ alkyl optionally substituted with one or more —NR¹R² or C₁-C₆ alkoxy;

Ar_{T} is selected from pyridinyl, phenyl, thiophenyl, pyrazolyl, imidazolyl, and tetrazolyl, wherein Ar_{T} is optionally substituted by one or more substituents independently selected from halogen, —NR⁷R⁸, C₁-C₆ alkyl, and C₁-C₆ alkoxy;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one or more substituents independently selected from (C₁-C₄ alkylene)-O—C(═O)C₁-C₆ alkyl, —C(═O)NR¹R², —C(═O)R¹², aryl, and C₁-C₆ alkyl-(aryl).

each R¹⁰ and R¹¹ is independently selected from hydrogen and C₁-C₆ alkyl optionally substituted with one or more of —C(═O)OH, —OH, aryl, hydroxyaryl, or heteroaryl; and

R¹² is selected from C₁-C₆ alkyl and aryl.

120. The compound of item 119, wherein:

Z is —C(═O)— or —CH₂—;

Ar_{C} is selected from phenylene and monocyclic heteroarylene; each substituted with one or more R_{C};

R_{C} is selected from —CN, —OH, C₁-C₆ alkoxy, C₁-C₆ alkyl, heteroaryl, aryl, —C(═O)OH, —C(═O)OR³;

each R¹ and R² is independently selected from hydrogen and C₁-C₆ alkyl;

each R³ is independently C₁-C₆alkyl optionally substituted with one or more —NR¹R²;

Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;

each R_{M} is hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one or more substituents independently selected from —C(═O)R¹² and aryl.

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, aryl, hydroxyaryl, and heteroaryl; and

R¹² is C₁-C₆ alkyl or aryl.

121. The compound of item 119 or 120, wherein:

Z is selected from —C(═O)— and —CH₂—;

Ar_{C} is arylene substituted with one R_{C};

R_{C} is selected from —C(═O)OH and tetrazolyl;

Ar_{T} is selected from pyridinyl, phenyl, thiophenyl, pyrazolyl, imidazolyl, and tetrazolyl, wherein Ar_{T} is optionally substituted by one or more of halogen, C₁-C₆ alkyl, and C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein heteroaryl is optionally substituted by one of —C(═O)R¹² or aryl;

each R¹⁰ and R¹¹ is independently selected from hydrogen and optionally substituted C₁-C₆alkyl; wherein the C₁-C₆ alkyl is optionally substituted by one or more substituents independently selected from —C(═O)OH, —OH, phenyl, hydroxyphenyl, and indolyl; and

R¹² is C₁-C₆ alkyl.

122. The compound of item 121, wherein Ar_{C} is phenylene.

123. The compound of item 121 or 122, wherein R_{L} is triazolyl optionally substituted by one of —C(═O)R¹² or aryl.

124. The compound of item 119 or 120, wherein:

Z is selected from —C(═O)— and —CH₂—;

Ar_{C} is heteroarylene substituted with one or two R_{C};

each R_{C} is independently selected from —CN, C₁-C₆ alkyl, and aryl;

Ar_{T} is phenyl optionally substituted by one or more substituents independently selected from halogen, C₁-C₆alkyl, or C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and C₁-C₆ alkyl optionally substituted by one or more substituents independently selected from —C(═O)OH, —OH, phenyl, hydroxyphenyl, and indolyl; and

R¹² is C₁-C₆ alkyl.

125. The compound of item 124, wherein Ar_{C} is thiophenylene.

126. The compound of item 124 or 125, wherein R_{L} is triazolyl optionally substituted by one of —C(═O)R¹² or aryl.

127. The compound of any one of items 124-126, wherein one of R_{C} is —CN.

128. The compound of item 119 or 120, wherein:

Z is selected from —C(═O)— and —CH₂—;

Ar_{C} is arylene substituted with one R_{C};

R_{C} is —C(═O)OR³;

R¹ and R² is independently selected from hydrogen and C₁-C₆ alkyl;

R³ is C₁-C₆ alkyl optionally substituted with one NR¹R²;

Ar_{T} is phenyl optionally substituted by one or more substituents independently selected from halogen, C₁-C₆ alkyl, and C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and C₁-C₆ alkyl optionally substituted by one or more substituents independently selected from —C(═O)OH, —OH, phenyl, hydroxyphenyl, and indolyl; and

R¹² is C₁-C₆ alkyl.

129. The compound of item 128, wherein Ar_{C} is phenylene.

130. The compound of item 128 or 129, wherein R_{L} is triazolyl optionally substituted by one of —C(═O)R¹² or aryl.

131. The compound of item 119 or 120, wherein:

Z is —C(═O)—;

Ar_{C} is arylene substituted with one R_{C};

R_{C} is selected from —C(═O)OH and tetrazolyl;

Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and C₁-C₆ alkyl optionally substituted by one or more substituents independently selected from —C(═O)OH, —OH, aryl, hydroxyaryl and heteroaryl; and

R¹² is C₁-C₆ alkyl.

132. The compound of item 131, wherein Ar_{C} is phenylene.

133. The compound of item 131 or 132, wherein R_{L} is triazolyl optionally substituted by one of —C(═O)R¹² or aryl.

134. The compound of item 119 or 120, wherein:

Z is —C(═O)—;

Ar_{C} is heteroarylene substituted with one or two R_{C};

each R_{C} is independently selected from —CN, C₁-C₆ alkyl, and aryl;

Ar_{T} is phenyl optionally substituted by one or more of halogen, C₁-C₆ alkyl, or C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, aryl, hydroxyaryl or heteroaryl; and

R¹² is C₁-C₆ alkyl.

135. The compound of item 134, wherein Ar_{C} is thiophenylene.

136. The compound of item 134 or 135, wherein R_{L} is triazolyl optionally substituted by one of —C(═O)R¹² or aryl.

137. The compound of any one of items 134-136, wherein one of R_{C} is —CN.

138. The compound of item 119 or 120, wherein:

Z is —C(═O)—;

Ar_{C} is arylene substituted with one R_{C};

R_{C} is —C(═O)OR³;

R¹ and R² is independently selected from hydrogen and C₁-C₆ alkyl;

R³ is C₁-C₆ alkyl optionally substituted with one —NR¹R²;

Ar_{T} is phenyl optionally substituted by one or more of halogen, C₁-C₆ alkyl, or C₁-C₆ alkoxy;

each R_{M} are hydrogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

R¹⁰ is selected from hydrogen and C₁-C₆ alkyl;

each R¹⁰ and R¹¹ is independently selected from hydrogen and optionally substituted C₁-C₆ alkyl; wherein the C₁-C₆ alkyl is optionally substituted by one or more substituents independently selected from —C(═O)OH, —OH, aryl, hydroxyaryl, and heteroaryl; and

R¹² is C₁-C₆ alkyl.

139. The compound of item 138, wherein Ar_{C} is phenylene.

140. The compound of item 138 or 139, wherein R_{L} is triazolyl optionally substituted by one of —C(═O)R¹² or aryl.

141. The compound of item 1, wherein the compound of Formula (I) is represented by Formula (V):

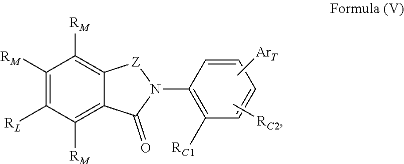

or a pharmaceutically acceptable salt thereof, wherein:

Z is —C(═O)— or —C(R^{a})(R^{b})—;

R^{a} and R^{b} are each independently selected from hydrogen, fluorine and methyl;

R_{C1} is selected from —OH, tetrazolyl, —C(═O)OH, and —C(═O)OR³;

R_{C2} is selected from hydrogen, halogen, —OH, C₁-C₆ alkyl, C₁-C₆ hydroxyalkyl and C₁-C₆ alkoxy;

R³ is C₁-C₆ alkyl optionally substituted with one or more substituent selected from —NR¹R² or C₁-C₆ alkoxy;

each R¹ and R² is independently selected from hydrogen and C₁-C₆ alkyl;

or R¹ and R² are taken together with the N to which they are attached to form a C₂-C₈ heterocycloalkyl;

Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

R¹⁰ is selected from hydrogen and C₁-C₆ alkyl;

R¹¹ is selected from hydrogen and optionally substituted C₁-C₆ alkyl; wherein the C₁-C₆ alkyl is optionally substituted with one or more of —C(═O)OH, —OH, aryl, hydroxyaryl, and heteroaryl; and

R¹² is selected from C₁-C₆ alkyl and aryl.

142. The compound of item 141, wherein R_{C1} is tetrazolyl or —C(═O)OH.

143. The compound of item 141, wherein R_{C1} is —C(═O)OR³.

144. The compound of any one of items 141-143, wherein R_{C2} is hydrogen.

145. The compound of any one of items 141-144, wherein Ar_{T} is selected from pyridinyl, phenyl and thiophenyl, each optionally substituted by one or more substituents independently selected from halogen, C₁-C₆ alkyl, and C₁-C₆ alkoxy.

146. The compound of any one of items 141-144, wherein Ar_{T} is pyrazolyl or imidazolyl each optionally substituted by methyl.

147. The compound of any one of items 141-146, wherein R_{L} is triazolyl optionally substituted by one of —C(═O)R¹² or aryl.

148. The compound of item 1, wherein the compound of Formula (I) is represented by Formula (VI):

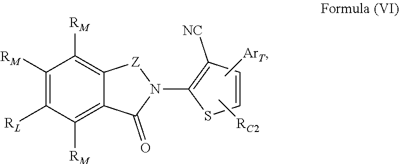

or a pharmaceutically acceptable salt thereof, wherein:

Z is —C(═O)— or —C(R^{a})(R^{b})—;

R^{a} and R^{b} are each independently selected from hydrogen, fluorine and methyl;

R_{C2} is selected from hydrogen, halogen, —OH, C₁-C₆ alkyl, C₁-C₆ hydroxyalkyl, C₁-C₆ alkoxy and aryl;

Ar_{T} is phenyl optionally substituted by one or more substituents independently selected from halogen, C₁-C₆ alkyl, and C₁-C₆ alkoxy;

each R_{M} is independently selected from hydrogen and halogen;

R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;

R¹⁰ is selected from hydrogen and C₁-C₆ alkyl;

R¹¹ is selected from hydrogen and optionally substituted C₁-C₆ alkyl; wherein the C₁-C₆ alkyl is optionally substituted with one or more of —C(═O)OH, —OH, aryl, hydroxyaryl, and heteroaryl; and

R¹² is selected from C₁-C₆ alkyl and aryl.

149. The compound of item 148, wherein R_{C2} is selected from C₁-C₆ alkyl and phenyl.

150. The compound of item 148 or 149, wherein Z is —C(═O)—.

151. The compound of item 148 or 149, wherein Z is —CH₂—.

152. The compound of any one of items 148-151, wherein each R_{M} is hydrogen.

153. The compound of any one of items 148-152, wherein R_{L} is monocyclic heteroaryl optionally substituted by one of —C(═O)R¹² or aryl.

154. The compound of any one of items 148-153, wherein R_{L} is tetrazolyl.

155. The compound of any one of items 148-153, wherein R_{L} is triazolyl optionally substituted by one of —C(═O)R¹² or aryl.

156. The compound of any one of items 148-152, wherein R_{L} is —C(═O)OH.

157. The compound of any one of items 148-152, wherein R_{L} is —C(═O)NR¹⁰R¹¹, wherein R¹⁰ is selected from hydrogen and C₁-C₆ alkyl; and R¹¹ is selected from hydrogen and C₁-C₆ alkyl (optionally substituted with one or more of —C(═O)OH, —OH, phenyl, hydroxyphenyl, or indolyl).

158. The compound of any one of items 148-152, wherein R_{L} is —C(═O)NHS(═O)₂R¹².

159. The compound of any one of items 141-158, wherein the compound or a pharmaceutically acceptable salt thereof is in the form of a prodrug.

160. The compound of item 159, wherein the prodrug comprises an ester moiety.

161. The compound of item 159, wherein the prodrug comprises an amide moiety.

162. A compound of Formula (VII):

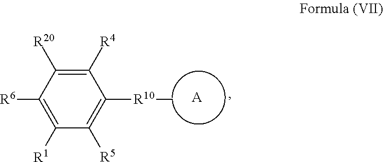

or a pharmaceutically acceptable salt thereof, wherein:

A is selected from:

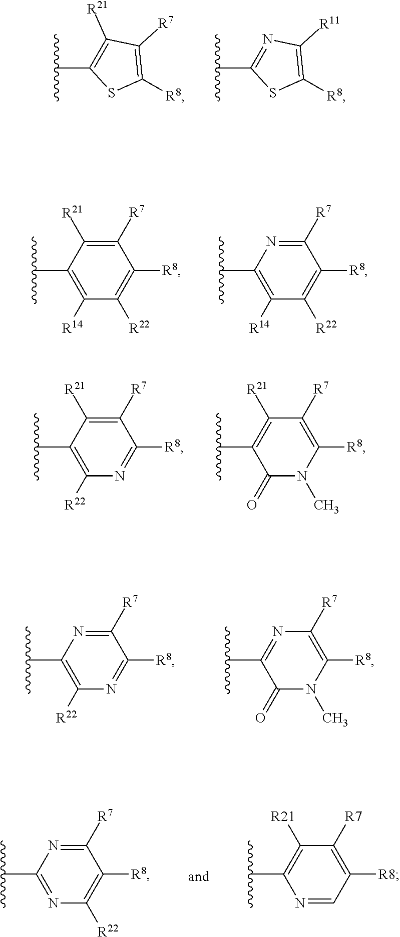

R¹ is selected from hydrogen, halogen, hydroxyl, C₁-C₆ alkyl, and C₁-C₆ alkoxy,

wherein the C₁-C₆ alkyl and C₁-C₆ alkoxy are optionally substituted with one or more halogens;

each R² and R³ is independently selected from hydrogen and C₁-C₆ alkyl,

wherein the C₁-C₆ alkyl is optionally substituted with one or more halogens;

or R² and R³ are taken together with the N to which they are attached to form a 3- to 10-membered heterocycle optionally substituted with one or more substituents independently selected from C₁-C₆ alkyl;

R⁴ is selected from hydrogen, halogen, C₁-C₆ alkyl, and C₁-C₆alkoxy,

wherein the C₁-C₆ alkyl and C₁-C₆ alkoxy are optionally substituted with one or more substituents independently selected from halogens;

R⁵ is selected from —C(═O)OR¹⁵, —C(═O)NR²R³, —S(═O)₂NR²R³, —C(═O)NHR¹⁵, —CH₂OH, 3-hydroxyoxetan-3-yl, and —NH₂;

R⁶ is selected from hydrogen, halogen, hydroxyl, 5-membered heteroaryl, C₁-C₆ alkyl, —C(═O)OR¹⁵, —C(═O)R¹², —C(═O)NHR¹⁵, and —C(═O)N═S(═X³)(CH₃)₂,

wherein the C₁-C₆ alkyl are optionally substituted with one or more R⁹, and

wherein 5-membered heteroaryl contains at least two heteroatoms and is optionally substituted with one or more substituents independently selected from R¹⁷;

R⁷ is selected from hydrogen, —NO₂, C₁-C₆ alkyl, C₁-C₆alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,

wherein the C₁-C₆ alkyl and C₁-C₆ alkoxy are optionally substituted with one or more halogens, and

wherein C₃-C₈ cycloalkyl, —O—C₃-C₈ cycloalkyl, 3- to 10-membered heterocycloalkyl, —O—(3- to 10-membered heterocycloalkyl), aryl and heteroaryl are optionally substituted with one or more R²⁴;

R⁸ is selected from hydrogen, —NO₂, C₁-C₆ alkyl, aryl, and heteroaryl,

wherein the C₁-C₆ alkyl and C₁-C₆ alkoxy are optionally substituted with one or more substituents independently selected at each occurrence from halogen; and

wherein aryl and heteroaryl are optionally substituted with one or more substituents independently selected at each occurrence from R²³;

or R⁷ and R⁸ are taken together to form a C₅-C₁₀ carbocycle or 5- to 10-membered heterocycle,

wherein C₅-C₁₀ carbocycle and 5- to 10-membered heterocycle are optionally substituted with one or more substituents independently selected from halogen, hydroxyl, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,

wherein the C₁-C₆ alkyl and C₁-C₆ alkoxy are optionally substituted with one or more halogens, and

wherein aryl, heteroaryl, C₃-C₈ cycloalkyl, —O—C₃-C₈ cycloalkyl, 3- to 10-membered heterocycloalkyl, and —O-(3- to 10-membered heterocycloalkyl) are optionally substituted with one or more R²³;

each R⁹ is independently selected from hydroxy and —COOH;

R¹⁰ is selected from —C(═O)—X¹—, —CH₂—X¹—, —X¹—C(═O)—, and —X¹—CH₂—;

R¹¹ is selected from hydrogen, —NO₂, C₁-C₆ alkyl, C₁-C₆alkoxy, C₃-C₈ cycloalkyl, —O—C₃-C₈ cycloalkyl, 3- to 10-membered heterocycloalkyl, and —O—(3- to 10-membered heterocycloalkyl),

wherein the C₁-C₆ alkyl and C₁-C₆ alkoxy are optionally substituted with one or more halogens, and

wherein C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;

R¹² is selected from alanine, arginine, asparagine, aspartic acid, asparagine, aspartic acid, cysteine, glutamic acid, glutamine, glutamine, glutamic acid, glycine, histidine, isoleucine, leucine, lysine, methionine, phenylalanine, proline, serine, threonine, tryptophan, tyrosine, and valine, wherein the point of attachment of R¹² is a nitrogen atom;

R¹⁴ is selected from hydrogen, halogen, hydroxyl, nitrile, —C(═O)CR¹⁵ and —C(═O)OR¹⁵;

each R¹⁵ is independently selected from hydrogen and C₁-C₆ alkyl, -heterocyclyl,

wherein the C₁-C₆ alkyl is optionally substituted with one or more substituents independently selected at each occurrence from —C(═O)NR²R³, -heterocyclyl, —NR²R³;

wherein the heterocyclyl is optionally substituted with one or more substituents independently selected at each occurrence from R² and R³.

R¹⁷ is selected from C₁-C₆ alkyl, aryl, and 6-membered heteroaryl,

wherein the C₁-C₆ alkyl is optionally substituted with one or more hydroxy, and

wherein aryl and 6-membered heteroaryl are optionally substituted with one or more substituents independently selected from halogen, —R², and —OR²;

R²⁰ is selected from hydrogen, halogen, hydroxyl, —COOH, —NC(═O)R², —OR², 5-membered heteroaryl, C₁-C₆ alkyl, —C(═O)N═S(═X³)(CH₃)₂, —CH₂(OH)CH₂OH and —NH—SO₂—R², wherein the 5-membered heteroaryl contains at least two heteroatoms, and

wherein the C₁-C₆ alkyl is optionally substituted with one or more substituents independently selected from 5-membered heteroaryl, wherein the 5-membered heteroaryl contains at least two heteroatoms;

R²¹ is selected from hydrogen and nitrile;

R²² is selected from hydrogen and hydroxy;

each R²³ is independently selected from halogen, C₁-C₆ alkyl, C₁-C₆ alkoxy,

wherein the C₁-C₆ alkyl and C₁-C₆ alkoxy are optionally substituted with one or more substituents independently selected from halogens;

each R²⁴ is independently selected from halogen, C₁-C₆ alkyl, C₁-C₆ alkoxy, 5-membered heteroaryl wherein the C₁-C₆ alkyl and C₁-C₆ alkoxy are optionally substituted with one or more substituents independently selected from halogens;

each X¹ is independently selected from —NR²— and —CR²R³—; and

each X³ is independently selected from NH and O.

163. The compound or salt of item 162, wherein R¹⁰ is selected from —C(═O)—X¹— or —X¹—C(═O)—.

164. The compound or salt of item 163, wherein the compound of Formula (VII) is represented by Formula (VIIA):

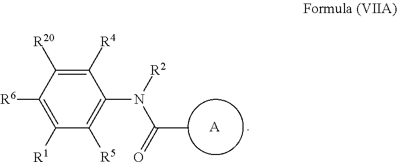

165. The compound or salt of any one of items 162-164, wherein A is selected from:

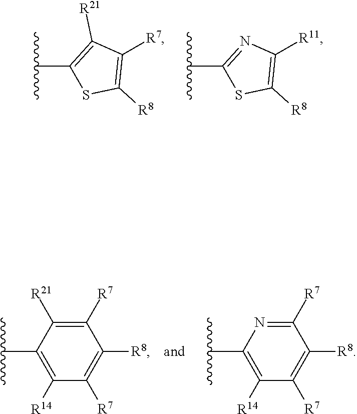

166. The compound or salt of any one of items 162-165, wherein A is selected from:

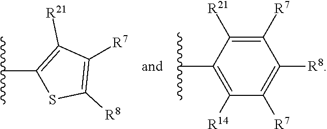

167. The compound or salt of any one of items 162-166, wherein A is

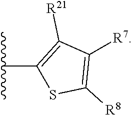

168. The compound or salt of any one of items 162-166, wherein A is

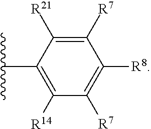

169. The compound or salt of any one of items 162-168, wherein R¹ is selected from hydrogen, halogen, and hydroxyl.

170. The compound or salt of item 169, wherein R¹ is hydrogen.

171. The compound or salt of item 169, wherein R¹ is halogen, wherein halogen is selected from F, Cl, and Br.

172. The compound or salt of any of items 162-171, wherein R⁸ is selected from —C(═O)OR¹⁵, —C(═O)NR²R³, and —C(═O)NHR¹⁵.

173. The compound or salt of item 172, wherein R⁸ is —C(═O)OR¹⁵.

174. The compound or salt of item 172, wherein R⁸ is —C(═O)NR²R³.

175. The compound or salt of any of items 162-174, wherein R⁶ is selected from hydrogen, halogen, hydroxyl, —C(═O)OR¹⁵, —C(═O)R¹², and —C(═O)NHR¹⁵.

176. The compound or salt of item 175, wherein R⁶ is —C(═O)OR¹⁵.

177. The compound or salt of item 175, wherein R⁶ is —C(═O)NHR¹⁵.

178. The compound or salt of any of items 162-177, wherein R⁷ is selected from hydrogen, and C₁-C₆ alkyl, wherein the C₁-C₆ alkyl and C₁-C₆ alkoxy are optionally substituted with one or more halogens.

179. The compound or salt of any of items 162-177, wherein R⁷ is selected from 3- to 10-membered heterocycloalkyl, —O—(3- to 10-membered heterocycloalkyl), aryl, and heteroaryl, wherein C₃-C₈ cycloalkyl, —O—C₃-C₈ cycloalkyl, 3- to 10-membered heterocycloalkyl, —O—(3- to 10-membered heterocycloalkyl), aryl and heteroaryl are optionally substituted with one or more R²³.

180. The compound or salt of item 179, wherein R⁷ is heteroaryl,

wherein the heteroaryl is optionally substituted with one or more R²³.

181. The compound or salt of item 179, wherein R⁷ is aryl,

wherein the aryl is optionally substituted with one or more R²³.

182. The compound or salt of item 181, wherein R⁷ is phenyl,

wherein the phenyl is optionally substituted with one or more R²³.

183. The compound or salt of item 182, wherein R⁷ is phenyl, wherein the phenyl is substituted by one or more halogens.

184. The compound or salt of any of items 162-183, wherein R⁸ is selected from hydrogen, C₁-C₃ alkyl, and heteroaryl,

wherein the C₁-C₃ alkyl is optionally substituted with one or more substituents independently selected at each occurrence from halogen; and

wherein the heteroaryl is optionally substituted with one or more substituents independently selected at each occurrence from R²³.

185. The compound or salt of item 164, wherein R⁸ is selected from hydrogen and C₁-C₃ alkyl.

186. The compound or salt of any one of items 162-185, wherein R¹¹ is selected from hydrogen, C₁-C₆ alkyl, and 3- to 10-membered heterocycloalkyl, wherein the C₁-C₆ alkyl and C₁-C₆ alkoxy are optionally substituted with one or more halogens, and wherein the 3- to 10-membered heterocycloalkyl is optionally substituted with one or more R²³.

187. The compound or salt of any one of items 162-186, wherein R¹² is selected from alanine, arginine, asparagine, aspartic acid, asparagine, aspartic acid, cysteine, glutamic acid, glutamine, glutamine, glutamic acid, and glycine, wherein the point of attachment of R¹² is a nitrogen atom.

188. The compound or salt of any one of items 162-186, wherein R¹² is selected from histidine, isoleucine, leucine, lysine, methionine, phenylalanine, proline, serine, threonine, tryptophan, tyrosine, and valine, wherein the point of attachment of R¹² is a nitrogen atom.

189. The compound or salt of any one of items 162-188, wherein R¹⁴ is selected from hydrogen and —C(═O)OR¹⁵, wherein R¹⁵ is selected from hydrogen and C₁-C₃ alkyl.

190. The compound of item 189, wherein R¹⁴ is —COOH.

191. The compound of any of items 162-190, wherein R²⁰ is selected from hydrogen, hydroxyl, and —COOH.

192. The compound of any of items 162-190, wherein R²⁰ is selected from 5-membered heteroaryl and C₁-C₆ alkyl,

wherein the 5-membered heteroaryl contains at least two heteroatoms, and

wherein the C₁-C₆ alkyl is optionally substituted with one or more substituents independently selected from 5-membered heteroaryl, wherein the 5-membered heteroaryl contains at least two heteroatoms.

193. The compound or salt of item 164, wherein the compound of Formula (VIIA) is represented by Formula (VIIB):

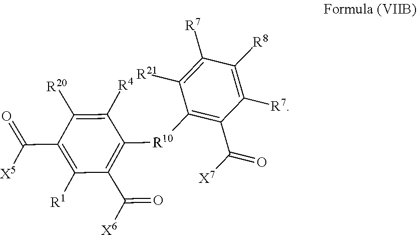

194. The compound of item 162, wherein the compound of Formula (VII) is selected from:

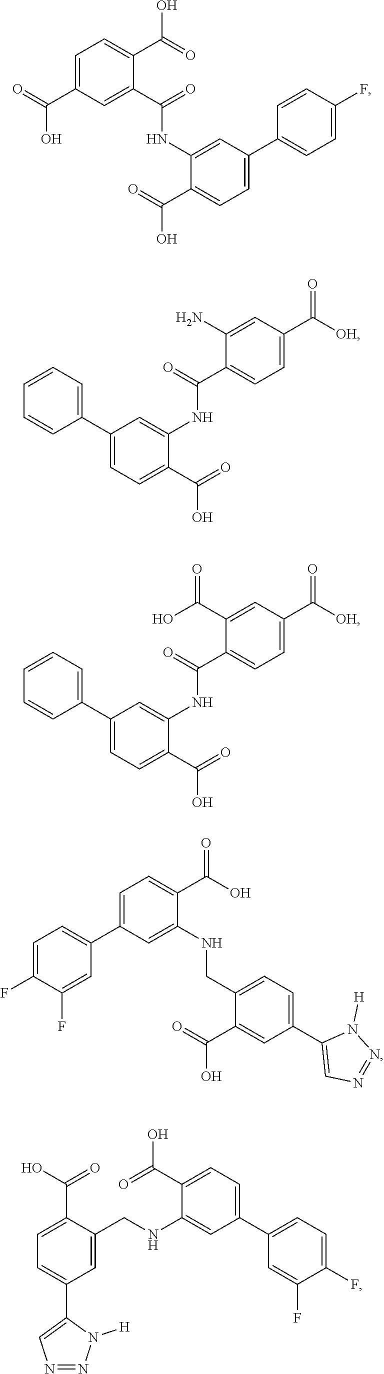

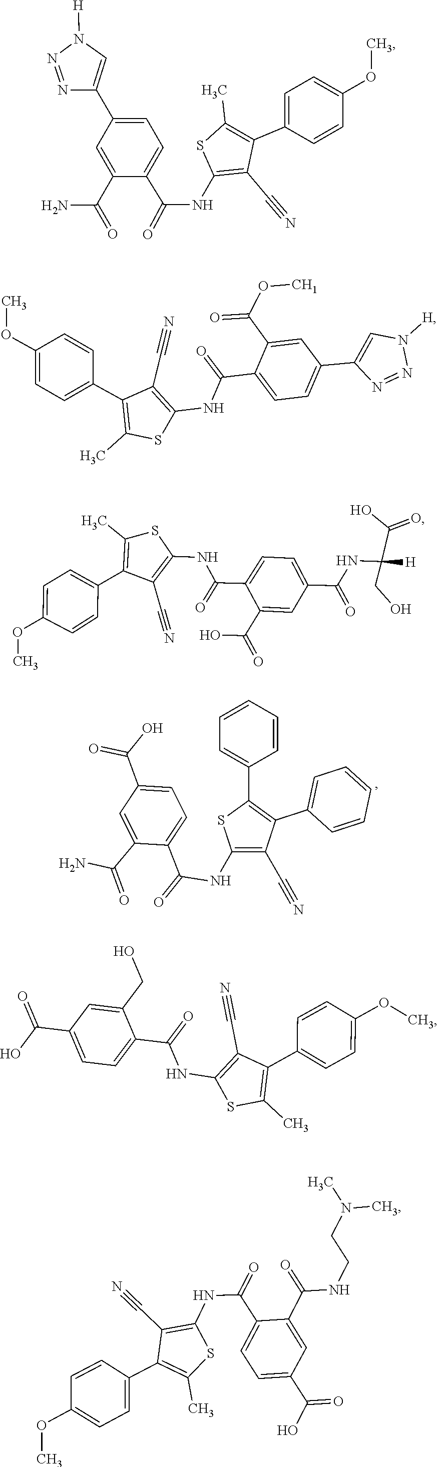

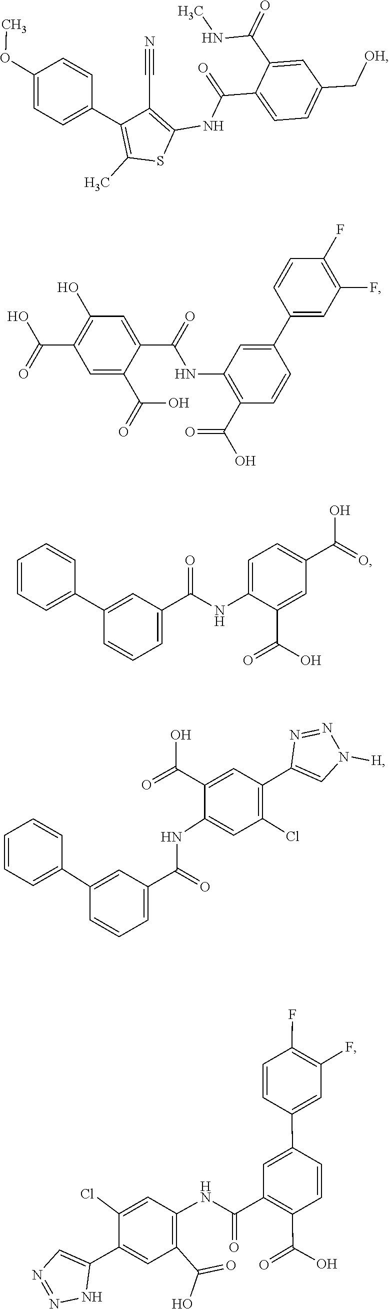

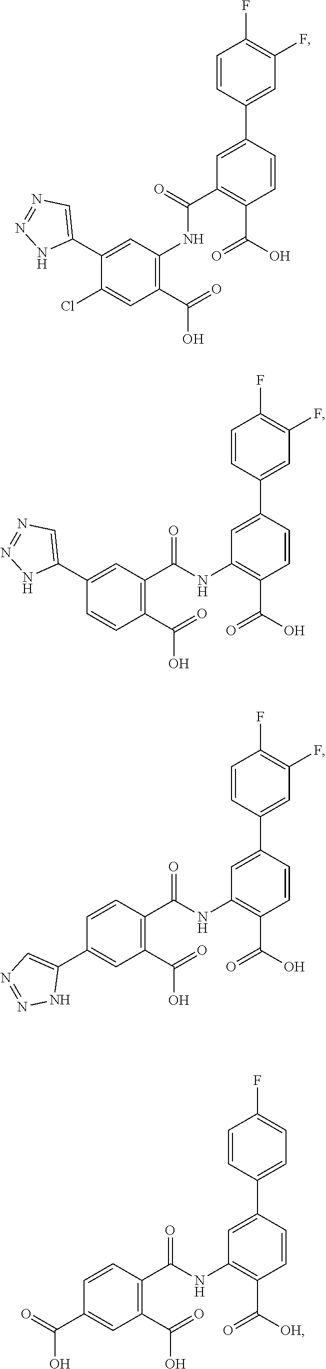 [SMILES: O=C(O)c1cc(Cl)c(-c2cnn[nH]2)cc1NC(=O)c1cc(-c2ccc(F)c(F)c2)ccc1C(=O)O ; O=C(O)c1ccc(-c2ccc(F)c(F)c2)cc1NC(=O)c1cc(-c2cnn[nH]2)ccc1C(=O)O ; O=C(O)c1ccc(-c2ccc(F)c(F)c2)cc1NC(=O)c1ccc(-c2cnn[nH]2)cc1C(=O)O ; O=C(O)c1ccc(C(=O)Nc2cc(-c3ccc(F)cc3)ccc2C(=O)O)c(C(=O)O)c1]

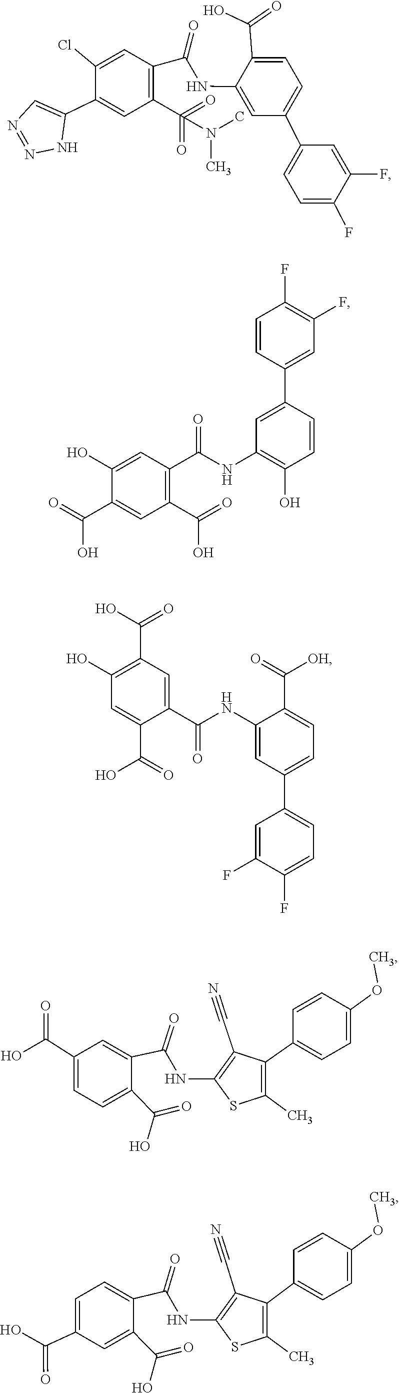

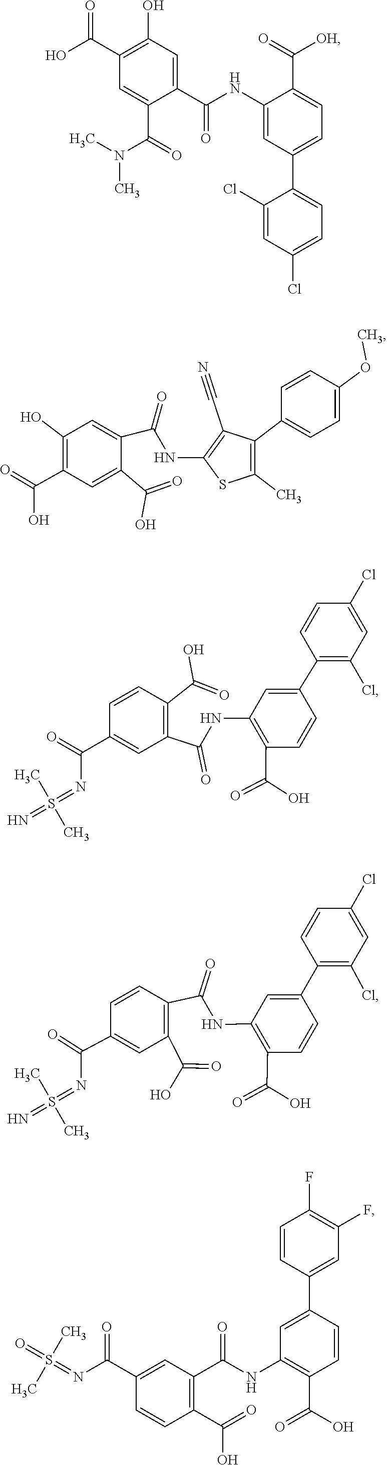 [SMILES: CN(C)C(=O)c1cc(C(=O)O)c(O)cc1C(=O)Nc1cc(-c2ccc(Cl)cc2Cl)ccc1C(=O)O ; COc1ccc(-c2c(C)sc(NC(=O)c3cc(O)c(C(=O)O)cc3C(=O)O)c2C#N)cc1 ; CS(C)(=N)=NC(=O)c1ccc(C(=O)O)c(C(=O)Nc2cc(-c3ccc(Cl)cc3Cl)ccc2C(=O)O)c1 ; CS(C)(=N)=NC(=O)c1ccc(C(=O)Nc2cc(-c3ccc(Cl)cc3Cl)ccc2C(=O)O)c(C(=O)O)c1 ; CS(C)(=O)=NC(=O)c1ccc(C(=O)O)c(C(=O)Nc2cc(-c3ccc(F)c(F)c3)ccc2C(=O)O)c1]

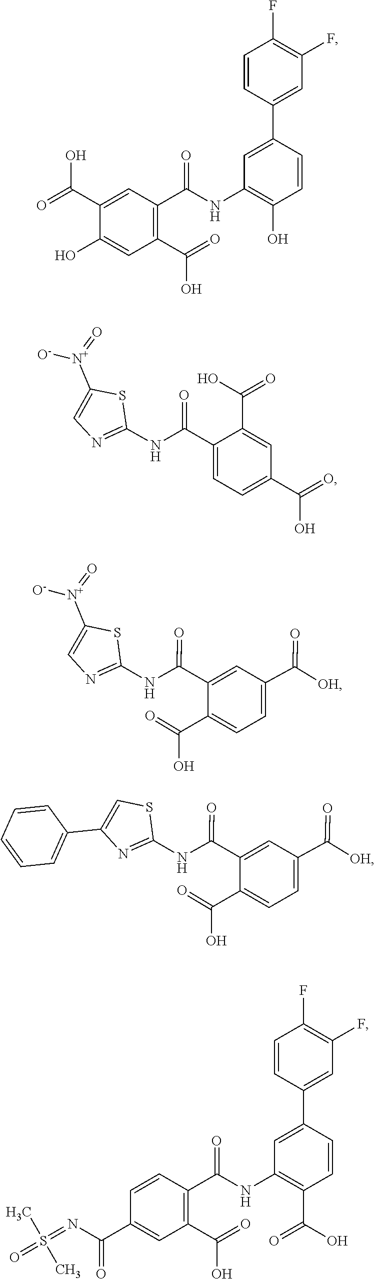 [SMILES: O=C(O)c1cc(C(=O)Nc2cc(-c3ccc(F)c(F)c3)ccc2O)c(C(=O)O)cc1O ; O=C(O)c1ccc(C(=O)Nc2ncc([N+](=O)[O-])s2)c(C(=O)O)c1 ; O=C(O)c1ccc(C(=O)O)c(C(=O)Nc2ncc([N+](=O)[O-])s2)c1 ; O=C(O)c1ccc(C(=O)O)c(C(=O)Nc2nc(-c3ccccc3)cs2)c1 ; CS(C)(=O)=NC(=O)c1ccc(C(=O)Nc2cc(-c3ccc(F)c(F)c3)ccc2C(=O)O)c(C(=O)O)c1]

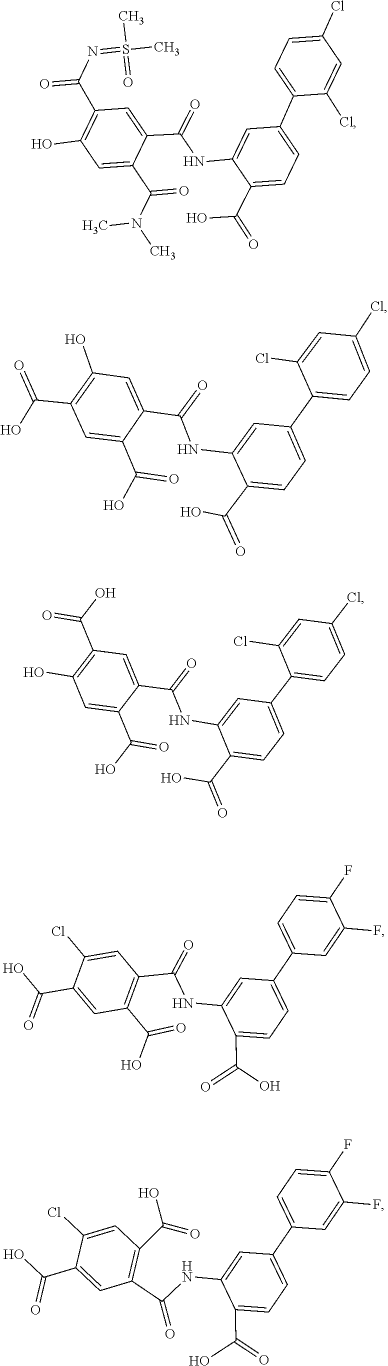 [SMILES: CN(C)C(=O)c1cc(O)c(C(=O)N=S(C)(C)=O)cc1C(=O)Nc1cc(-c2ccc(Cl)cc2Cl)ccc1C(=O)O ; O=C(O)c1cc(C(=O)O)c(C(=O)Nc2cc(-c3ccc(Cl)cc3Cl)ccc2C(=O)O)cc1O ; O=C(O)c1cc(C(=O)Nc2cc(-c3ccc(Cl)cc3Cl)ccc2C(=O)O)c(C(=O)O)cc1O ; O=C(O)c1cc(C(=O)O)c(C(=O)Nc2cc(-c3ccc(F)c(F)c3)ccc2C(=O)O)cc1Cl ; O=C(O)c1cc(C(=O)Nc2cc(-c3ccc(F)c(F)c3)ccc2C(=O)O)c(C(=O)O)cc1Cl]

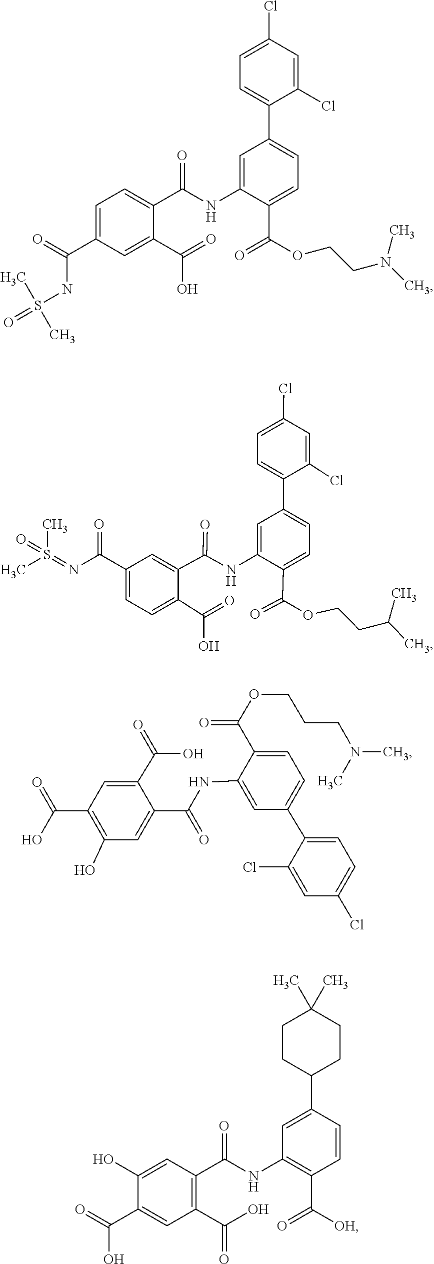

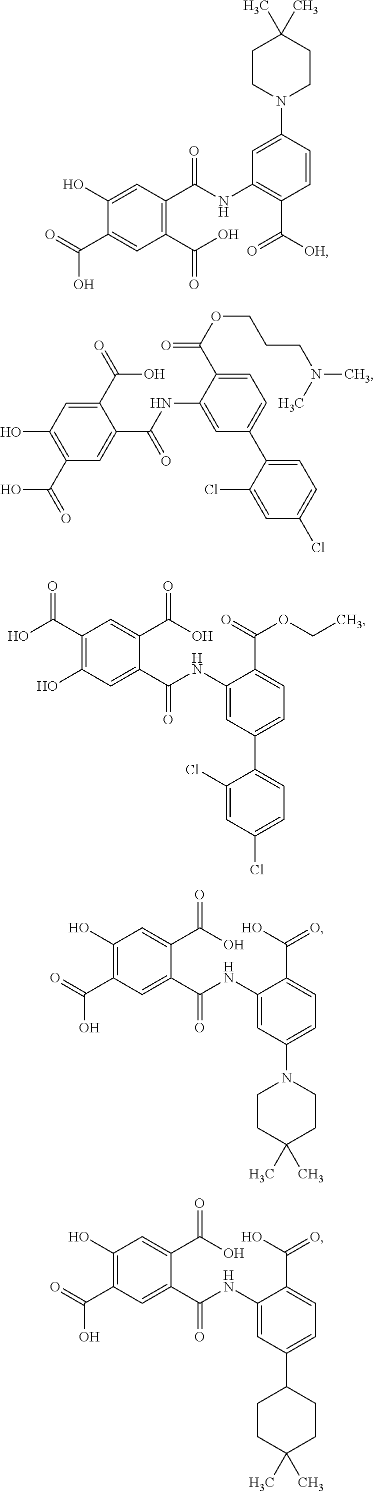

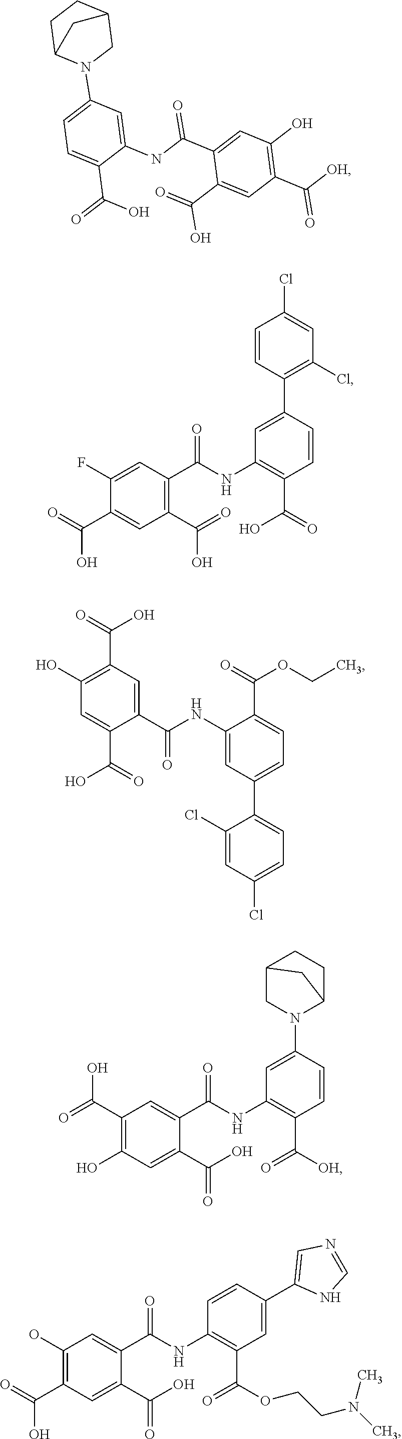

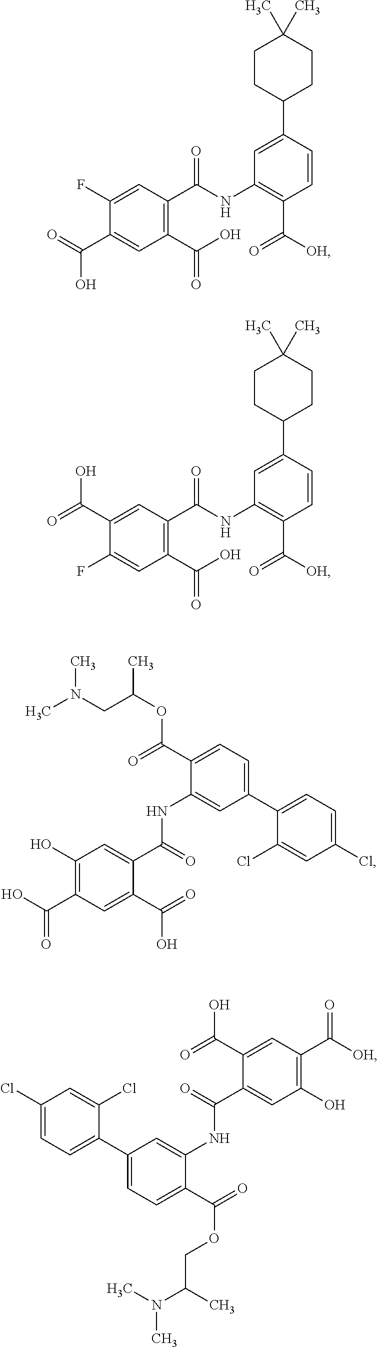 [SMILES: CC1(C)CCC(c2ccc(C(=O)O)c(NC(=O)c3cc(F)c(C(=O)O)cc3C(=O)O)c2)CC1 ; CC1(C)CCC(c2ccc(C(=O)O)c(NC(=O)c3cc(C(=O)O)c(F)cc3C(=O)O)c2)CC1 ; CC(CN(C)C)OC(=O)c1ccc(-c2ccc(Cl)cc2Cl)cc1NC(=O)c1cc(O)c(C(=O)O)cc1C(=O)O ; CC(COC(=O)c1ccc(-c2ccc(Cl)cc2Cl)cc1NC(=O)c1cc(O)c(C(=O)O)cc1C(=O)O)N(C)C]

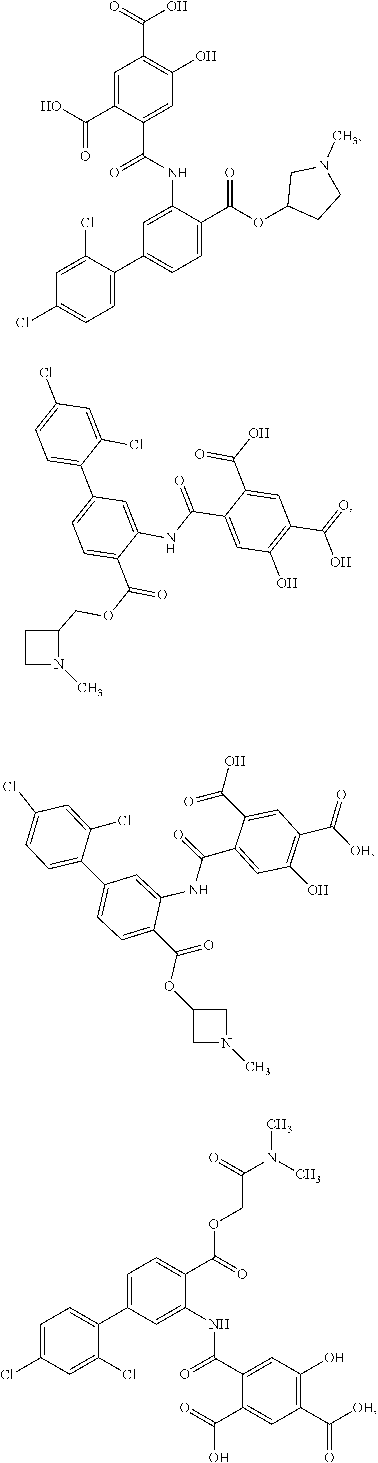 [SMILES: CN1CCC(OC(=O)c2ccc(-c3ccc(Cl)cc3Cl)cc2NC(=O)c2cc(O)c(C(=O)O)cc2C(=O)O)C1 ; CN1CCC1COC(=O)c1ccc(-c2ccc(Cl)cc2Cl)cc1NC(=O)c1cc(O)c(C(=O)O)cc1C(=O)O ; CN1CC(OC(=O)c2ccc(-c3ccc(Cl)cc3Cl)cc2NC(=O)c2cc(O)c(C(=O)O)cc2C(=O)O)C1 ; CN(C)C(=O)COC(=O)c1ccc(-c2ccc(Cl)cc2Cl)cc1NC(=O)c1cc(O)c(C(=O)O)cc1C(=O)O]

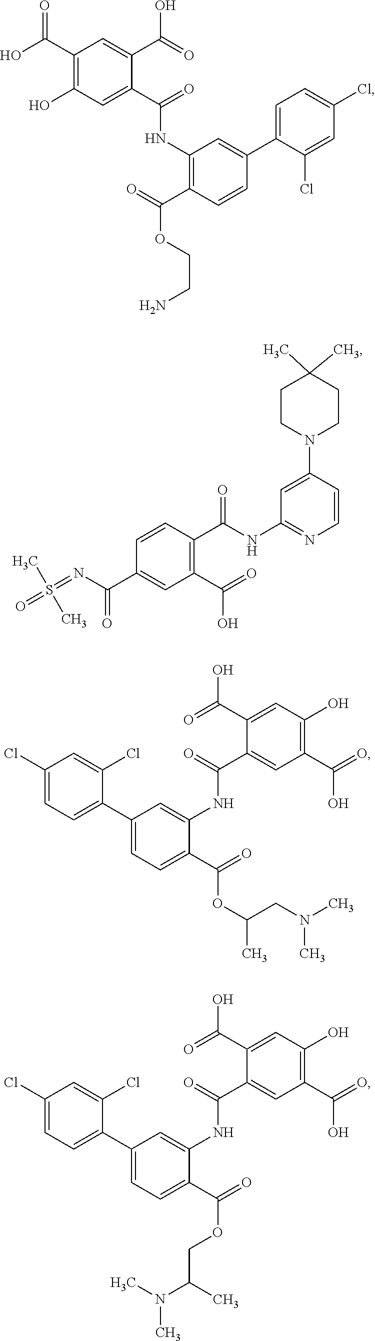 [SMILES: NCCOC(=O)c1ccc(-c2ccc(Cl)cc2Cl)cc1NC(=O)c1cc(O)c(C(=O)O)cc1C(=O)O ; CC1(C)CCN(c2ccnc(NC(=O)c3ccc(C(=O)N=S(C)(C)=O)cc3C(=O)O)c2)CC1 ; CC(CN(C)C)OC(=O)c1ccc(-c2ccc(Cl)cc2Cl)cc1NC(=O)c1cc(C(=O)O)c(O)cc1C(=O)O ; CC(COC(=O)c1ccc(-c2ccc(Cl)cc2Cl)cc1NC(=O)c1cc(C(=O)O)c(O)cc1C(=O)O)N(C)C]

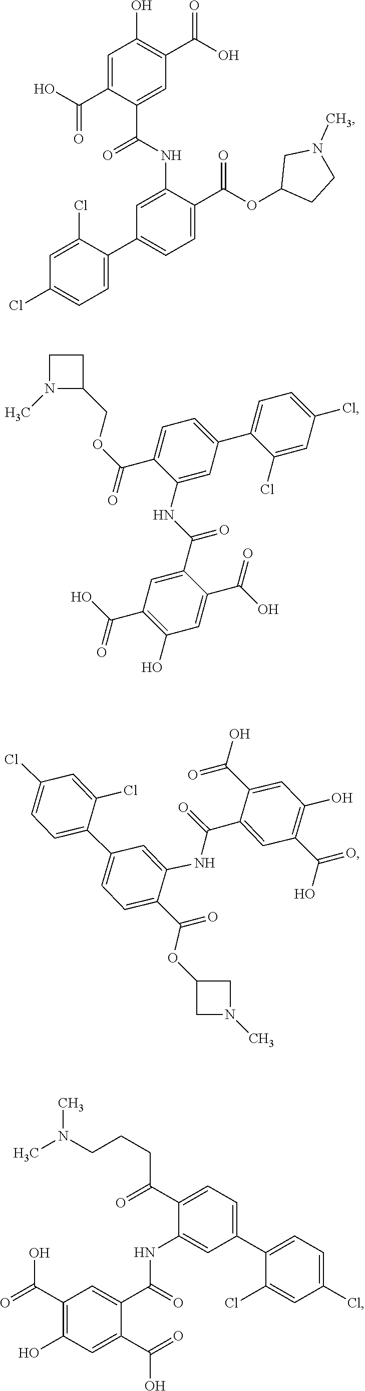 [SMILES: CN1CCC(OC(=O)c2ccc(-c3ccc(Cl)cc3Cl)cc2NC(=O)c2cc(C(=O)O)c(O)cc2C(=O)O)C1 ; CN1CCC1COC(=O)c1ccc(-c2ccc(Cl)cc2Cl)cc1NC(=O)c1cc(C(=O)O)c(O)cc1C(=O)O ; CN1CC(OC(=O)c2ccc(-c3ccc(Cl)cc3Cl)cc2NC(=O)c2cc(C(=O)O)c(O)cc2C(=O)O)C1 ; CN(C)CCCC(=O)c1ccc(-c2ccc(Cl)cc2Cl)cc1NC(=O)c1cc(C(=O)O)c(O)cc1C(=O)O]

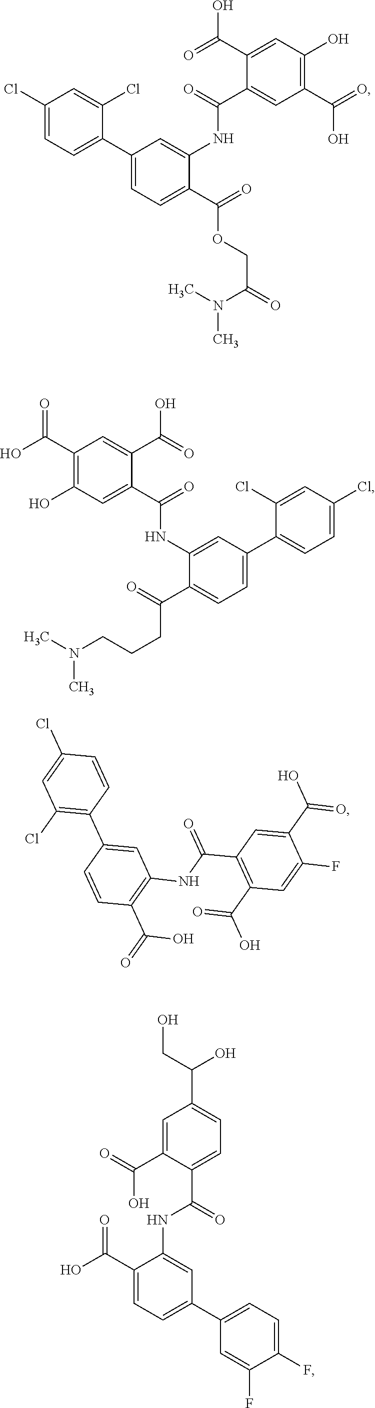

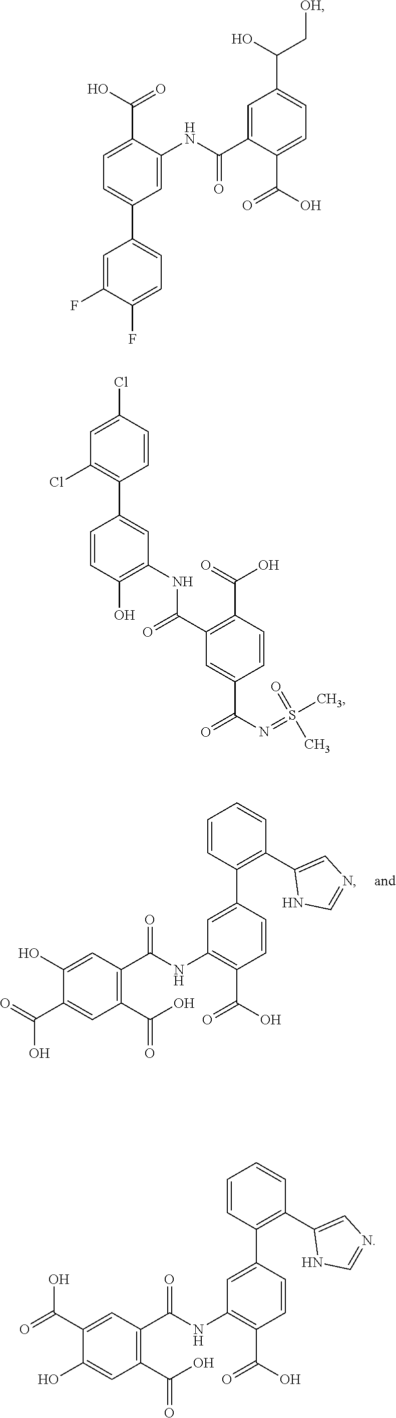

195. The compound of any one of items 162-194 or its pharmaceutically acceptable salt, wherein the compound or a pharmaceutically acceptable salt thereof is in the form of a prodrug.

196. The compound of item 195, wherein the prodrug comprises an ester moiety.

197. The compound of item 195, wherein the prodrug comprises an amide moiety.

198. A pharmaceutically acceptable acid addition salt of

compound of any one of the items 1 to 197, or a hydrate or solvate thereof, wherein the pharmaceutically acceptable acid addition salts thereof selected from the salts obtained with hydrochloric acid, hydrobromic acid, sulphuric acid, phosphoric acid, methanesulphonic acid, acetic acid, fumaric acid, succinic acid, lactic acid, citric acid, tartaric acid and maleic acid.

199. A derivative, N-oxide, solvate, tautomer, stereoisomer, racemate,

physiologically acceptable salt, including mixtures thereof in all ratios of compound of any one of the items 1 to 198.

200. A pharmaceutical composition comprising a compound of any one of items 1-199 and one or more pharmaceutically acceptable carrier.

To avoid any doubts, wordings “1-199” and alike and “1 to 199” and alike have an equal meaning, meaning any one of the items 1 to 199, such as 1, 2, 3 . . . 199 etc.

201. A pharmaceutical composition comprising a compound of any one of items 1-199 in combination with another therapeutic agent, and optionally, one or more pharmaceutically acceptable carriers.

202. The pharmaceutical composition of any one of preceding items, further comprising a second therapeutic agent.

203. The pharmaceutical composition of any one of preceding items, wherein the second therapeutic agent is an anti-cancer agent.

204. The compound of any one of items 1-199 for use for manufacturing a medicament for treating a disease or condition for which glycolysis inhibition has beneficial effect.

205. The compound of any one of items 1-199 for use for manufacturing a medicament for treating a cancer.

206. The compound of any one of items 1-199 for use for manufacturing a medicament for the treatment or prophylaxis of a disease or condition for which inhibition of PFKFB3 and/or PFKFB4 has beneficial effect.

207. The compound of any one of items 1-199 for use for manufacturing an inhibitor of glycolysis.

208. The compound of any one of items 1-199 for use for manufacturing an inhibitor of angiogenesis.

209. The compound of any one of items 1-199 for use for manufacturing an inhibitor of PFKFB3 and/or PFKFB4.

210. The compound of any one of items 1-199 for use for manufacturing an inhibitor of PFKFB3 and/or PFKFB4 kinase activity.

211. Compound of any one of items 1-199 for use as a modulator of PFKFB3 and/or PFKFB4.

212. Compound of any one of items 1-199 for use as an inhibitor of PFKFB3 and/or PFKFB4.

213. Compound of any one of items 1-199 for use as a modulator of PFKFB3 and/or PFKFB4 kinase activity.

214. Compound of any one of items 1-199 for use as an inhibitor of PFKFB3 and/or PFKFB4 kinase activity.

215. The compound of any one of items 1-199 or a pharmaceutical composition of any one of items 200 to 203 for use for the treatment or prophylaxis of diseases or conditions where the modulation of PFKFB3 and/or PFKFB4 has beneficial effect.

216. The compound of any one of items 1-199 or a pharmaceutical composition of any one of items 200 to 203 for use for the treatment or prophylaxis of disease or condition for which glycolysis inhibition has beneficial effect.

217. The compound of any one of items 1-199 or a pharmaceutical composition of any one of items 200 to 203 for use for the treatment or prophylaxis of disease or condition for which angiogenesis inhibition has beneficial effect.

218. The compound of any one of items 1-199 for use as an inhibitor of glycolysis.

219. The compound of any one of items 1-199 for use for the treatment of cancer.

220. The compound of any one of items 1-199 for use for the treatment of solid tumor.

221. The compound of any one of items 1-199 for use in treatment of a hematological cancer.

222. Use of a pharmaceutical composition of item 162 or 163 for the treatment of cancer.

223. The compound of any one of items 1-199 or a pharmaceutical composition of any one of items 200 to 203 for use for the treatment of bone cancer.

224. The compound of any one of items 1-199 or a pharmaceutical composition of any one of items 200 to 203 for use for the treatment of osteosarcoma.

225. The compound of any one of items 1-199 or a pharmaceutical composition of any one of items 200 to 203 for use for the treatment of bone cancer by administering such compound or composition.

226. The compound of any one of items 1-199 or a pharmaceutical composition of any one of items 200 to 203 for use for the treatment of at least one of the following: kidney cancer, colon cancer, pancreatic cancer, lung cancer, breast cancer, liver cancer, lymphoma, leukemia and myeloma

227. The compound of any one of items 1-199 or a pharmaceutical composition of any one of items 200 to 203 for use for the treatment of triple negative breast cancer.

228. The compound of any one of items 1-199 or a pharmaceutical composition of any one of items 200 to 203 for use for the treatment of least one of the following: atypical teratoid rhabdoid tumor, anal cancer, astrocytoma, vaginal cancer, extrahepatic bile duct cancer, intraocular melanoma, hairy cell leukemia, hepatocellular liver cancer, gestational trophoblastic disease, germ cell tumor, hypopharyngeal cancer, histiocytosis, histiocytosis Langerhans, high-grade astrocytoma, astrocytoma, glioma, brain stem glioma, invasive lobular carcinoma, gastrointestinal carcinoid tumor, gastrointestinal stromal tumor, cutaneous T-cell lymphoma, peripheral T-cell lymphoma, non-specific lymphoma mantle cell, lymphogranulomatosis, colorectal cancer, craniopharyngioma, leukemia, mast cell leukemia, Burkitt's lymphoma, Hodgkin's lymphoma, Waldenström's macroglobulinemia (lymphoplasmacytic lymphoma), small bowel cancer, mastocytosis, malignant mesothelioma, melanoma, small-cell carcinoma (small-cell lung cancer), metastatic squamous neck cancer, myelodysplastic/myeloproliferative neoplasms, myelodysplastic syndrome, acute myeloid leukemia, chronic myelogenous leukemia, chronic myeloproliferative disease, multiple myeloma (plasma cell myeloma or Kahler's disease), male breast cancer, nasal cell carcinoma, neuroblastoma, non-small cell lung cancer, non-Hodgkin's lymphoma, Wilms tumor, osteosarcoma, malignant fibrous histiocytoma of bone, acute lymphoblastic leukemia, acute myeloid leukemia, papillomatosis, paraganglioma, parathyroid carcinoma, transitional cell cancer of the renal pelvis, transitional cell cancer of the ureter, pleuropulmonary blastoma, squamous cell carcinoma, renal cell carcinoma, ductal carcinoma in situ, rhabdomyosarcoma, vulvar cancer, eye cancer, head and neck cancer, throat cancer, laryngeal cancer, lip and oral cancer, stomach cancer, gall bladder cancer, bile duct cancer, skin cancer, cancer of the adrenal cortex, bone cancer, uterine cancer, Merkel carcinoma, bladder cancer, nasopharyngeal cancer, esophageal cancer, penile cancer, nasal cavity cancer, paranasal sinus cancer, renal pelvis cancer, ureter cancer, renal cancer, Papillary renal cell carcinoma, prostate cancer, rectal cancer, oral cancer, salivary gland cancer, cancer of the urethra, cancer of the cervix, thyroid cancer, endometrial cancer, cancer of the central nervous system, testis cancer, ovarian cancer, retinoblastoma, sarcoma, Kaposi's sarcoma, uterine sarcoma, soft tissue sarcoma, Ewing's sarcoma, high-grade astrocytoma, astrocytoma, cardiac tumor, Sezary syndrome, pharyngeal cancer, pheochromocytoma, fibrous histiocytoma of bone, chordoma, chronic myeloproliferative disorder, chronic lymphocytic leukemia, ependymoma, erythroleukemia, and esthesioneuroblastoma.

229. The compound of any one of items 1-199 or a pharmaceutical composition of any one of items 200 to 203 for use to enhance the effect of radiation treatment of cancer.

230. The compound of any one of items 1-199 or a pharmaceutical composition of any one of items 200 to 203 for use to enhance the effect of radiation treatment of bone cancer.

231. The compound of any one of items 1-199 or a pharmaceutical composition of any one of items 200 to 203 for use to enhance the effect of radiation treatment of osteosarcoma.

232. The compound of any one of items 1-199 or a pharmaceutical composition of any one of items 200 to 203 for use to enhance the effect of radiation treatment of cancer, wherein such compound is administered prior to radiation treatment.

233. The compound of any one of items 1-199 or a pharmaceutical composition of any one of items 200 to 203 for use to enhance the effect of radiation treatment of cancer, wherein cancer type is selected from any one of preceding items.

234. The compound of any one of items 1-199 or a pharmaceutical composition of any one of items 200 to 203 for use to decrease the ability of the cancer cells to repair their DNA.

235. The compound of any one of items 1-199 or a pharmaceutical composition of any one of items 200 to 203 for use to sensitize cancer cell towards cytostatic and/or radiation therapy.

236. The compound of any one of items 1-199 or a pharmaceutical composition of any one of items 200 to 203 for use to sensitize cancer cell towards cytostatic and/or radiation therapy.

237. The compound of any one of items 1-199 or a pharmaceutical composition of any one of items 200 to 203 for use in treatment of neoplasm sensitive to inhibition of PFKFB3 or/and PFKFB4.

238. The compound of any one of items 1-199 or a pharmaceutical composition of any one of items 200 to 203 for use for the treatment of neoplasm sensitive to inhibition of glycolysis.

239. The compound of any one of items 1-199 or a pharmaceutical composition of any one of items 200 to 203 for use for the inhibition of angiogenesis.

240. The compound of any one of items 1-199 or a pharmaceutical composition of any one of items 200 to 203 for use for the treatment of an autoimmune disease.

241. The compound of any one of items 1-199 or a pharmaceutical composition of any one of items 200 to 203 for use for the treatment of an autoimmune disease selected from systemic lupus erythematosus, scleroderma, graft-versus-host disease, or transplanted organ rejection.

242. The compound of any one of items 1-199 or a pharmaceutical composition of any one of items 200 to 203 for use for the treatment of psoriasis.

243. The compound of any one of items 1-199 or a pharmaceutical composition of any one of items 200 to 203 for use for the treatment of rheumatoid arthritis.

244. The compound of any one of items 1-199 or a pharmaceutical composition of any one of items 200 to 203 for use for the treatment of an inflammatory disorder.

245. The compound of any one of items 1-199 or a pharmaceutical composition of any one of items 200 to 203 for use for the treatment of arthritis.

246. The compound of any one of items 1-199 or a pharmaceutical composition of any one of items 200 to 203 for use in the treatment of inflammatory bowel disease.

247. The compound of any one of items 1-199 or a pharmaceutical composition of any one of items 200 to 203 for use in the treatment of atherosclerosis.

248. The compound of any one of items 1-199 or a pharmaceutical composition of any one of items 200 to 203 for use to decrease atherosclerotic inflammation and/or its clinical consequences.

249. The compound of any one of items 1-199 or a pharmaceutical composition of any one of items 200 to 203 for use for the treatment of cystic fibrosis.

250. The compound of any one of items 1-199 or a pharmaceutical composition of any one of items 200 to 203 for use for the treatment of a metabolic disease.

251. The compound of any one of items 1-199 or a pharmaceutical composition of any one of items 200 to 203 for use for the treatment of glucose metabolism disorder.

252. The compound of any one of items 1-199 or a pharmaceutical composition of any one of items 200 to 203 for use for the treatment of hyperlactatemia

253. The compound of any one of items 1-199 or a pharmaceutical composition of any one of items 200 to 203 for use for the prophylaxis of cancer.

254. The compound of any one of the items 1-199 for use as an immunosuppressive agent.

255. The compound of any one of the items 1-199 for use as a T cell immunosuppressive agent.

256. The compound of any one of the items 1-199 for use or a pharmaceutical composition of any one of items 200 to 203 for treating a viral disease.

257. The compound of any one of the items 1-199 for use or a pharmaceutical composition of any one of items 200 to 203 for prophylaxis of a viral disease.

258. The compound of any one of the items 1-199 for use or a pharmaceutical composition of any one of items 200 to 203 for treating influenza.

259. The compound of any one of the items 1-199 for use or a pharmaceutical composition of any one of items 200 to 203 for treating influenza A.

260. The compound of any one of the items 1-199 for use or a pharmaceutical composition of any one of items 200 to 203 for prophylaxis of influenza.

261. The compound of any one of the items 1-199 for use or a pharmaceutical composition of any one of items 200 to 203 for prophylaxis of influenza A.

262. The compound of any one of the items 1-199 for use or a pharmaceutical composition of any one of items 200 to 203 as an anti-inflammatory agent.

263. The compound of any one of the items 1-199 for use for inhibition the glycolysis in a cell.

264. Use of a compound according to any one of the items 1-199 or a pharmaceutical composition of any one of items 200 to 203 as an anti-cancer agent.

265. The compound of any one of items 1-199 for use as modulator of a process modulated by PFKFB3 and/or by PFKFB4. 266. The compound of any one of items 1-199 for use as an inhibitor of PFK2 kinase activity.

267. The compound of any one of items 1-199 for use for reducing glycolytic flux in a cell.

268. The compound of any one of items 1-199 or a pharmaceutical composition of any one of items 200 to 203 for use for treating of a proliferative disease.

269. The compound of any one of items 1-199 or a pharmaceutical composition of any one of items 200 to 203 for use for the prophylaxis of at least one of the following: an autoimmune disease, an inflammatory disorder, a metabolic disease, and a proliferative disease.

270. The compound of any one of items 1-199 or a pharmaceutical composition of any one of items 200 to 203 for use for the prophylaxis of at least one of the following diseases: kidney cancer, colon cancer, pancreatic cancer, lung cancer, breast cancer, liver cancer, lymphoma, leukemia, myeloma, a hematological cancer, breast cancer, triple negative breast cancer, atypical teratoid rhabdoid tumor, anal cancer, astrocytoma, vaginal cancer, extrahepatic bile duct cancer, intraocular melanoma, hairy cell leukemia, hepatocellular liver cancer, gestational trophoblastic disease, germ cell tumor, hypopharyngeal cancer, histiocytosis, histiocytosis Langerhans, glioma, brain stem glioma, invasive lobular carcinoma, gastrointestinal carcinoid tumor, gastrointestinal stromal tumor, cutaneous T-cell lymphoma, peripheral T-cell lymphoma, non-specific lymphoma mantle cell, lymphogranulomatosis, colorectal cancer, craniopharyngioma, leukemia, mast cell leukemia, Burkitt's lymphoma, Hodgkin's lymphoma, Waldenström's macroglobulinemia (lymphoplasmacytic lymphoma), small bowel cancer, mastocytosis, malignant mesothelioma, melanoma, small-cell carcinoma (small-cell lung cancer), metastatic squamous neck cancer, myelodysplastic/myeloproliferative neoplasms, myelodysplastic syndrome, acute myeloid leukemia, chronic myelogenous leukemia, chronic myeloproliferative disease, multiple myeloma (plasma cell myeloma or Kahler's disease), male breast cancer, nasal cell carcinoma, neuroblastoma, non-small cell lung cancer, non-Hodgkin's lymphoma, Wilms tumor, osteosarcoma, malignant fibrous histiocytoma of bone, acute lymphoblastic leukemia, acute myeloid leukemia, papillomatosis, paraganglioma, parathyroid carcinoma, transitional cell cancer of the renal pelvis, transitional cell cancer of the ureter, pleuropulmonary blastoma, squamous cell carcinoma, renal cell carcinoma, ductal carcinoma in situ, rhabdomyosarcoma, vulvar cancer, eye cancer, head and neck cancer, throat cancer, laryngeal cancer, lip and oral cancer, stomach cancer, gall bladder cancer, bile duct cancer, skin cancer, cancer of the adrenal cortex, bone cancer, uterine cancer, Merkel carcinoma, bladder cancer, nasopharyngeal cancer, esophageal cancer, penile cancer, nasal cavity cancer, paranasal sinus cancer, renal pelvis cancer, ureter cancer, renal cancer, Papillary renal cell carcinoma, prostate cancer, rectal cancer, oral cancer, salivary gland cancer, cancer of the urethra, cancer of the cervix, thyroid cancer, endometrial cancer, cancer of the central nervous system, testis cancer, ovarian cancer, retinoblastoma, sarcoma, Kaposi's sarcoma, uterine sarcoma, soft tissue sarcoma, Ewing's sarcoma, cardiac tumor, Sezary syndrome, pharyngeal cancer, pheochromocytoma, fibrous histiocytoma of bone, chordoma, chronic myeloproliferative disorder, chronic lymphocytic leukemia, ependymoma, erythroleukemia, and esthesioneuroblastoma.

271. The compound of any one of items 1-199 or a pharmaceutical composition of any one of items 200 to 203 for use for the prophylaxis of disease selected from systemic lupus erythematosus, scleroderma, graft-versus-host disease, transplanted organ rejection, psoriasis, rheumatoid arthritis, arthritis, inflammatory bowel disease, atherosclerosis, atherosclerotic inflammation, at least one of the clinical consequences of atherosclerotic inflammation, cystic fibrosis, hyperlactatemia, cerebral ischemia, and neurological insult.

272. A method of inhibition of the glycolysis in a cell, comprising contacting the cell with an effective amount of a compound of any one of items 1-199.

273. A method of modulating the activity of PFKFB3 and/or PFKFB4 in a cell, comprising contacting the cell with an effective amount of a compound of any one of items 1-199 or a pharmaceutical composition of any one of items 200 to 203.

274. A method of inhibition of PFKFB3 and/or PFKFB4 in a cell, comprising contacting the cell with an effective amount of a compound of any one of items 1-199.

275. A method of inhibiting 6-phosphofructo-2-kinase/fructose-2,6-bisphosphatase 3 (PFKFB3), the method comprising contacting PFKFB3 with an effective amount of a compound of any one of items 1-199.

276. A method of inhibiting 6-phosphofructo-2-kinase/fructose-2,6-bisphosphatase 4 (PFKFB4), the method comprising contacting PFKFB4 with an effective amount of a compound of any one of items 1-199.

277. A method of inhibiting of PFKFB3 and/or PFKFB4 in a cell, the method comprising contacting a cell with an effective amount of a compound of any one of items 1-199.

278. A method of treatment or prophylaxis of disease or condition for which glycolysis inhibition has beneficial effect, comprising administering to a subject in need thereof an effective amount of a compound of any one of items 1-199 or a pharmaceutical composition of any one of items 200 to 203.

279. A method of treatment or prophylaxis of disease or condition for which PFKFB3 and/or PFKFB4 inhibition has beneficial effect, comprising administering to a subject in need thereof an effective amount of a compound of any one of items 1-199 or a pharmaceutical composition of any one of items 200 to 203.

280. A method of reducing glycolytic flux in a cell, the method comprising contacting the cell with an effective amount of a compound of any one of items 1-199.

281. A method of treating an autoimmune disease, an inflammatory disorder, a metabolic disease, a viral disease, a proliferative disease comprising administering to a subject in need thereof an effective amount of a compound of any one of items 1-199 or a pharmaceutical composition of any one of items 200 to 203.

282. A method of reducing proliferative capacity in a cell, the method comprising contacting the cell with an effective amount of a compound of any one of items 1-199.

283. A method of increasing of cell antioxidant capacity, the method comprising contacting the cell with an effective amount of a compound of any one of items 1-199.

284. A method of enhancing the effect of radiation treatment of cancer, the method comprising administering to a subject in need thereof an effective amount of a compound of any one of items 1-199 or a pharmaceutical composition of any one of items 200 to 203.

285. A method of enhancing the effect of radiation treatment of bone cancer, the method comprising administering to a subject in need thereof an effective amount of a compound of any one of items 1-199 or a pharmaceutical composition of any one of items 200 to 203.

286. A method of enhancing the effect of radiation treatment of osteosarcoma, the method comprising administering to a subject in need thereof an effective amount of a compound of any one of items 1-199 or a pharmaceutical composition of any one of items 200 to 203.

287. A method of enhancing the effect of radiation treatment of cancer, the method comprising administering to a subject in need thereof an effective amount of a compound of any one of items 1-199 or a pharmaceutical composition of any one of items 200 to 203, wherein such compound is administered prior to radiation treatment.

288. A method of enhancing the effect of radiation treatment of cancer, the method comprising administering to a subject in need thereof an effective amount of a compound of any one of items 1-199 or a pharmaceutical composition of any one of items 200 to 203, wherein cancer type is selected from any one of preceding items.

289. A method of decreasing the ability of the cancer cells to repair their DNA, the method comprising contacting the cell with an effective amount of a compound of any one of items 1-199.

290. A method of sensitizing cancer cell towards cytostatic and/or radiation therapy, the method comprising contacting the cell with an effective amount of a compound of any one of items 1-199 or a pharmaceutical composition of any one of items 200 to 203.

291. A method of treatment of neoplasm sensitive to inhibition of PFKFB3 or/and PFKFB4, the method comprising administering to a subject in need thereof an effective amount of a compound of any one of items 1-199 or a pharmaceutical composition of any one of items 200 to 203.

292. A method of treatment of neoplasm sensitive to inhibition of glycolysis, the method comprising administering to a subject in need thereof an effective amount of a compound of any one of items 1-199 or a pharmaceutical composition of any one of items 200 to 203.

293. A method of reducing proliferative capacity in a cancer cell, the method comprising contacting the cancer cell with an effective amount of a compound of any one of items 1-199.

294. A method of treatment of a cancer, the method comprising administering to the subject an effective amount of a compound of any one of items 1-199 or a pharmaceutical composition of any one of items 200 to 203.

295. A method of treatment of cancer comprising administering to a subject in need thereof an effective amount of a compound of any one of items 1-199 or a pharmaceutical composition of any one of items 200 to 203.

296. A method of treatment of solid tumor comprising administering to a subject in need thereof an effective amount of a compound of any one of items 1-199 or a pharmaceutical composition of any one of items 200 to 203.

297. A method of treatment of a hematological cancer, comprising administering to a subject in need thereof an effective amount of a compound of any one of items 1-199 or a pharmaceutical composition of any one of items 200 to 203.

298. A method of treatment of cancer selected from kidney cancer, colon cancer, pancreatic cancer, lung cancer, breast cancer, triple negative breast cancer, liver cancer, lymphoma, leukemia, myeloma, comprising administering to a subject in need thereof an effective amount of a compound of any one of items 1-199 or a pharmaceutical composition of any one of items 200 to 203.

299. A method of treatment of cancer selected from: atypical teratoid rhabdoid tumor, anal cancer, astrocytoma, vaginal cancer, extrahepatic bile duct cancer, intraocular melanoma, hairy cell leukemia, hepatocellular liver cancer, gestational trophoblastic disease, germ cell tumor, hypopharyngeal cancer, histiocytosis, histiocytosis Langerhans, high-grade astrocytoma, astrocytoma, glioma, brain stem glioma, invasive lobular carcinoma, gastrointestinal carcinoid tumor, gastrointestinal stromal tumor, cutaneous T-cell lymphoma, peripheral T-cell lymphoma, non-specific lymphoma mantle cell, lymphogranulomatosis, colorectal cancer, craniopharyngioma, leukemia, mast cell leukemia, Burkitt's lymphoma, Hodgkin's lymphoma, Waldenström's macroglobulinemia (lymphoplasmacytic lymphoma), small bowel cancer, mastocytosis, malignant mesothelioma, melanoma, small-cell carcinoma (small-cell lung cancer), metastatic squamous neck cancer, myelodysplastic/myeloproliferative neoplasms, myelodysplastic syndrome, acute myeloid leukemia, chronic myelogenous leukemia, chronic myeloproliferative disease, multiple myeloma (plasma cell myeloma or Kahler's disease), male breast cancer, nasal cell carcinoma, neuroblastoma, non-small cell lung cancer, non-Hodgkin's lymphoma, Wilms tumor, osteosarcoma, malignant fibrous histiocytoma of bone, acute lymphoblastic leukemia, acute myeloid leukemia, papillomatosis, paraganglioma, parathyroid carcinoma, transitional cell cancer of the renal pelvis, transitional cell cancer of the ureter, pleuropulmonary blastoma, squamous cell carcinoma, renal cell carcinoma, ductal carcinoma in situ, rhabdomyosarcoma, vulvar cancer, eye cancer, head and neck cancer, throat cancer, laryngeal cancer, lip and oral cancer, stomach cancer, gall bladder cancer, bile duct cancer, skin cancer, cancer of the adrenal cortex, bone cancer, uterine cancer, Merkel carcinoma, bladder cancer, nasopharyngeal cancer, esophageal cancer, penile cancer, nasal cavity cancer, paranasal sinus cancer, renal pelvis cancer, ureter cancer, renal cancer, Papillary renal cell carcinoma, prostate cancer, rectal cancer, oral cancer, salivary gland cancer, cancer of the urethra, cancer of the cervix, thyroid cancer, endometrial cancer, cancer of the central nervous system, testis cancer, ovarian cancer, retinoblastoma, sarcoma, Kaposi's sarcoma, uterine sarcoma, soft tissue sarcoma, Ewing's sarcoma, cardiac tumor, Sezary syndrome, pharyngeal cancer, pheochromocytoma, fibrous histiocytoma of bone, chordoma, chronic myeloproliferative disorder, chronic lymphocytic leukemia, ependymoma, erythroleukemia, esthesioneuroblastoma, comprising administering to a subject in need thereof an effective amount of a compound of any one of items 1-199 or a pharmaceutical composition of any one of items 200 to 203.

300. A method of treatment of bone cancer comprising administering to a subject in need thereof an effective amount of a compound of any one of items 1-199 or a pharmaceutical composition of any one of items 200 to 203.

301. A method of treatment of osteosarcoma comprising administering to a subject in need thereof an effective amount of a compound of any one of items 1-199 or a pharmaceutical composition of any one of items 200 to 203.

302. A method for treating of a cancer, which comprises administering an effective amount of a compound of any one of items 1-199 or a pharmaceutical composition of any one of items 200 to 203 and at least one other anti-cancer medication.

303. A method for treating of a cancer, which comprises administering an effective amount of a compound of any one of items 1-199 or a pharmaceutical composition of any one of items 200 to 203 and at least one other anti-cancer medication selected from Irinotecan and Sunitinib.

304. A method for treating of a cancer, which comprises administering an effective amount of a compound of any one of items 1-199 or a pharmaceutical composition of any one of items 200 to 203 and at least one other anti-cancer medication, wherein anti-cancer medication is targeted therapy.

305. A method for treating of a cancer, which comprises administering an effective amount of a compound of any one of items 1-199 or a pharmaceutical composition of any one of items 200 to 203 and at least one other anti-cancer medication, wherein anti-cancer medication is immunotherapy.

306. A method of treating a cancer cell, comprising contacting the cancer cell with an effective amount of a compound of any one of items 1-199.

307. A method of inducing an apoptosis of cancer cell, comprising contacting the cancer cell with an effective amount of a compound of any one of items 1-199.

308. A method of inhibition of angiogenesis comprising administering to a subject in need thereof an effective amount of a compound of any one of items 1-199 or a pharmaceutical composition of any one of items 200 to 203.

309. A method of treatment of an autoimmune disease comprising administering to a subject in need thereof an effective amount of a compound of any one of items 1-199 or a pharmaceutical composition of any one of items 200 to 203.

310. A method of treatment of an autoimmune disease selected from psoriasis, systemic lupus erythematosus, scleroderma, graft-versus-host disease, or transplanted organ rejection comprising administering to a subject in need thereof an effective amount of a compound of any one of items 1-199 or a pharmaceutical composition of any one of items 200 to 203.

311. A method of treatment inflammation, comprising administering to a subject in need thereof an effective amount of a compound of any one of items 1-199 or a pharmaceutical composition of any one of items 200 to 203.

312. A method of treatment of disorder selected from atherosclerosis, arthritis, rheumatoid arthritis, cystic fibrosis, inflammatory bowel disease, cerebral ischemia, neurological insult, influenza, inflammation, comprising administering to a subject in need thereof an effective amount of a compound of any one of items 1-199 or a pharmaceutical composition of any one of items 200 to 203.

313. A method of decreasing atherosclerotic inflammation and/or at least one of its clinical consequences comprising administering to a subject in need thereof an effective amount of a compound of any one of items 1-199 or a pharmaceutical composition of any one of items 200 to 203.

314. A method of treatment of metabolic disease comprising administering to a subject in need thereof an effective amount of a compound of any one of items 1-199 or a pharmaceutical composition of any one of items 200 to 203.

315. A method of treatment of glucose metabolism disorder comprising administering to a subject in need thereof an effective amount of a compound of any one of items 1-199 or a pharmaceutical composition of any one of items 200 to 203.

316. A method of treatment of hyperlactatemia comprising administering to a subject in need thereof an effective amount of a compound of any one of items 1-199 or a pharmaceutical composition of any one of items 200 to 203.

317. A method of immunosuppression, comprising the step of administering to a patient in need thereof a compound of any one of items 1-199 or a pharmaceutical composition of any one of items 200 to 203.

318. A method of prophylaxis of cancer, an autoimmune disease, an inflammatory disorder, a metabolic disease, a viral disease, a proliferative disease comprising administering to a subject in need thereof an effective amount of a compound of any one of items 1-199 or a pharmaceutical composition of any one of items 200 to 203.

319. A method of prophylaxis of neoplasm sensitive to inhibition of PFKFB3 or/and PFKFB4, the method comprising administering to a subject in need thereof an effective amount of a compound of any one of items 1-199 or a pharmaceutical composition of any one of items 200 to 203.

320. A method of prophylaxis of neoplasm sensitive to inhibition of glycolysis, the method comprising administering to a subject in need thereof an effective amount of a compound of any one of items 1-199 or a pharmaceutical composition of any one of items 200 to 203.

321. A method of prophylaxis of a cancer, the method comprising administering to the subject an effective amount of a compound of any one of items 1-199 or a pharmaceutical composition of any one of items 200 to 203

322. A method of prophylaxis of cancer selected from solid tumors, namely kidney, colon, pancreas, lung, breast and liver cancers, and hematologic neoplasms, namely lymphoma, leukemia and myeloma, a hematological cancer, breast cancer, comprising administering to a subject in need thereof an effective amount of a compound of any one of items 1-199 or a pharmaceutical composition of any one of items 200 to 203.

323. A method of prophylaxis of a hematological cancer, comprising administering to a subject in need thereof an effective amount of a compound of any one of items 1-199 or a pharmaceutical composition of any one of items 200 to 203.

324. A method of prophylaxis of cancer selected from kidney cancer, colon cancer, pancreatic cancer, lung cancer, breast cancer, triple negative breast cancer, liver cancer, lymphoma, leukemia, myeloma, comprising administering to a subject in need thereof an effective amount of a compound of any one of items 1-199 or a pharmaceutical composition of any one of items 200 to 203.

325. A method of prophylaxis of cancer selected from: atypical teratoid rhabdoid tumor, anal cancer, astrocytoma, vaginal cancer, extrahepatic bile duct cancer, intraocular melanoma, hairy cell leukemia, hepatocellular liver cancer, gestational trophoblastic disease, germ cell tumor, hypopharyngeal cancer, histiocytosis, histiocytosis Langerhans, glioma, high-grade astrocytoma, astrocytoma, brain stem glioma, invasive lobular carcinoma, gastrointestinal carcinoid tumor, gastrointestinal stromal tumor, cutaneous T-cell lymphoma, peripheral T-cell lymphoma, non-specific lymphoma mantle cell, lymphogranulomatosis, colorectal cancer, craniopharyngioma, leukemia, mast cell leukemia, Burkitt's lymphoma, Hodgkin's lymphoma, Waldenström's macroglobulinemia (lymphoplasmacytic lymphoma), small bowel cancer, mastocytosis, malignant mesothelioma, melanoma, small-cell carcinoma (small-cell lung cancer), metastatic squamous neck cancer, myelodysplastic/myeloproliferative neoplasms, myelodysplastic syndrome, acute myeloid leukemia, chronic myelogenous leukemia, chronic myeloproliferative disease, multiple myeloma (plasma cell myeloma or Kahler's disease), male breast cancer, nasal cell carcinoma, neuroblastoma, non-small cell lung cancer, non-Hodgkin's lymphoma, Wilms tumor, osteosarcoma, malignant fibrous histiocytoma of bone, acute lymphoblastic leukemia, acute myeloid leukemia, papillomatosis, paraganglioma, parathyroid carcinoma, transitional cell cancer of the renal pelvis, transitional cell cancer of the ureter, pleuropulmonary blastoma, squamous cell carcinoma, renal cell carcinoma, ductal carcinoma in situ, rhabdomyosarcoma, vulvar cancer, eye cancer, head and neck cancer, throat cancer, laryngeal cancer, lip and oral cancer, stomach cancer, gall bladder cancer, bile duct cancer, skin cancer, cancer of the adrenal cortex, bone cancer, uterine cancer, Merkel carcinoma, bladder, nasopharyngeal cancer, esophageal cancer, penile cancer, nasal cavity cancer, paranasal sinus cancer, renal pelvis cancer, ureter cancer, renal cancer, Papillary renal cell carcinoma, prostate cancer, rectal cancer, oral cancer, salivary gland cancer, cancer of the urethra, cancer of the cervix, thyroid cancer, endometrial cancer, cancer of the central nervous system, testis cancer, ovarian cancer, retinoblastoma, sarcoma, Kaposi's sarcoma, uterine sarcoma, soft tissue sarcoma, Ewing's sarcoma, cardiac tumor, Sezary syndrome, pharyngeal cancer, pheochromocytoma, fibrous histiocytoma of bone, chordoma, chronic myeloproliferative disorder, chronic lymphocytic leukemia, ependymoma, erythroleukemia, and esthesioneuroblastoma, comprising administering to a subject in need thereof an effective amount of a compound of any one of items 1-199 or a pharmaceutical composition of any one of items 200 to 203.

326. A method for prophylaxis of a cancer, which comprises administering an effective amount of a compound of any one of items 1-199 or a pharmaceutical composition of any one of items 200 to 203 and at least one other cancer prophylactic medication.

327. A method of prophylaxis of an autoimmune disease comprising administering to a subject in need thereof an effective amount of a compound of any one of items 1-199 or a pharmaceutical composition of any one of items 200 to 203.

328. A method of prophylaxis of a disease selected from psoriasis, systemic lupus erythematosus, scleroderma, graft-versus-host disease, and transplanted organ rejection comprising administering to a subject in need thereof an effective amount of a compound of any one of items 1-199 or a pharmaceutical composition of any one of items 200 to 203.

329. A method of prophylaxis of an inflammatory disorder comprising administering to a subject in need thereof an effective amount of a compound of any one of items 1-199 or a pharmaceutical composition of any one of items 200 to 203.

330. A method of prophylaxis of disorder selected from atherosclerosis, arthritis, rheumatoid arthritis, cystic fibrosis, inflammatory bowel disease, cerebral ischemia, neurological insult, influenza, inflammation, comprising administering to a subject in need thereof an effective amount of a compound of any one of items 1-199 or a pharmaceutical composition of any one of items 200 to 203.

331. A method of prophylaxis of metabolic disease comprising administering to a subject in need thereof an effective amount of a compound of any one of items 1-199 or a pharmaceutical composition of any one of items 200 to 203.

332. A method of prophylaxis of glucose metabolism disorder comprising administering to a subject in need thereof an effective amount of a compound of any one of items 1-199 or a pharmaceutical composition of any one of items 200 to 203.

333. A method of prophylaxis of hyperlactatemia comprising administering to a subject in need thereof an effective amount of a compound of any one of items 1-199 or a pharmaceutical composition of any one of items 200 to 203.

334. A method of manufacturing a medication, comprising the compound of any one of items 1-199 for use as an active ingredient.

335. A method of manufacturing a medication, comprising the compound of any one of items 1-199 for use as an active ingredient, wherein the medicament is at least one of the following: medicament for treating a disease or condition for which glycolysis inhibition has beneficial effect, medicament for treating a cancer, a medicament for the treatment or prophylaxis of a disease or condition for which inhibition of kinase activity of PFKFB3 and/or PFKFB4 has beneficial effect, an inhibitor of glycolysis, an inhibitor of angiogenesis.

336. A kit for treating a PFKFB3 and/or PFKFB4-mediated condition, comprising (a) a pharmaceutical composition comprising a compound of any one of items 1 to 199; and (b) instructions for use.

337. A kit for treating a cancer, comprising (a) a pharmaceutical composition comprising a compound of any one of items 1 to 199; and (b) instructions for use.

338. Compound of formula (VIII):

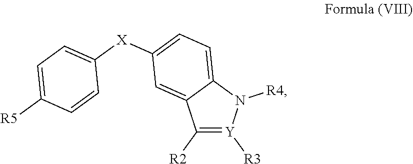

or a pharmaceutically acceptable salt thereof, wherein: each X is independently selected from —O—, —S—, —NR⁷— or —CR⁷R⁸—;

each Y is independently selected from C or N;

each R⁷ and R⁸ is independently selected from hydrogen and C₁-C₆ alkyl, C₁-C₆ alkoxy, wherein said alkyl is optionally substituted with one or more halogens;

R² is selected from hydrogen, halogens, nitrile and

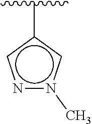

R³ is selected from hydrogen and —NR⁷R⁸

R⁴ is selected from hydrogen, C₁-C₆ alkyl, C₁-C₆ alkoxy, aryl, heteroaryl, C₃-C₈ cycloalkyl, 10-membered heterocycloalkyl,

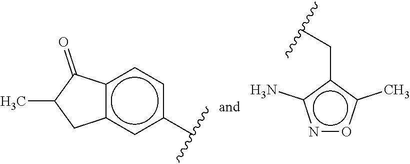

wherein the C₁-C₆alkyl and C₁-C₆alkoxy are optionally substituted with one or more substituents independently selected at each occurrence from halogens, —C(═O)NR⁷R⁸ and R²; and

wherein heteroaryl, C₃-C₈ cycloalkyl, —O—C₃-C₈ cycloalkyl, 3- to 10-memberedheterocycloalkyl and —O—(3- to 10-memberedheterocycloalkyl) are optionally substituted with one or more R²;

R⁵ is selected from

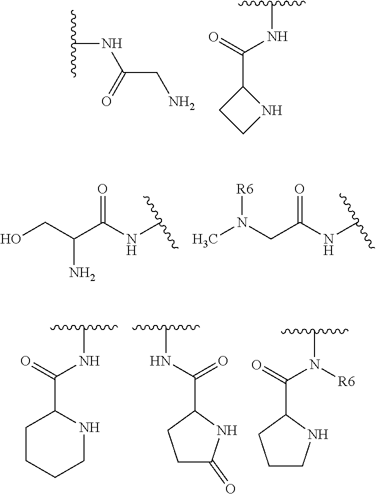

R⁶ is selected from hydrogen and C₁-C₆ alkyl for use as neuroprotector.

339. Compound for use as neuroprotector of item 338, wherein compound is (2RS)—N-[4-({3-cyano-1-[(3,5-dimethyl-1,2-oxazol-4-yl)methyl]-1H-indol-5-yl}oxy)phenyl]pyrrolidine-2-carboxamide.

340. Compound for use as neuroprotector of item 338, wherein compound is N-[4-({3-cyano-1-[(3,5-dimethyl-1,2-oxazol-4-yl)methyl]-1H-indol-5-yl}oxy)phenyl]pyrrolidine-2-carboxamide.

341. Compound for use as neuroprotector of item 338, wherein compound is (2S)—N-[4-({3-cyano-1-[(3,5-dimethyl-1,2-oxazol-4-yl)methyl]-1H-indol-5-yl}oxy)phenyl]pyrrolidine-2-carboxamide.

342. Compound for use as neuroprotector of item 338, wherein compound is selected from (2RS)—N-[4-({3-cyano-1-[(3,5dimethyl-1,2-oxazol-4-yl)methyl]-1H-indol-5-yl}oxy)phenyl]pyrrolidine-2-carboxamide, N-[4-({3-cyano-1-[(3,5-dimethyl-1,2-oxazol-4-yl)methyl]-1H-indol-5-yl}oxy)phenyl]pyrrolidine-2-carboxamide, (2S)—N-[4-({3-cyano-1-[(3,5-dimethyl-1,2-oxazol-4-yl)methyl]-1H-indol-5-yl}oxy)phenyl]pyrrolidine-2-carboxamide, 2S)—N-{4-[(3-cyano-1-ethyl-1H-indol-5-yl)oxy]phenyl}-5-oxopyrrolidine-2-carboxamide, 2-amino-N-(4-{[3-(1-methyl-1H-pyrazol-4-yl)-1H-indol-5-yl]oxy}phenyl)acetamide, (2S)—N-(4-{[1-methyl-3-(1-methyl-1H-pyrazol-4-yl)-1H-indol-5-yl]oxy}phenyl)pyrrolidine-2-carboxamide, (2S)—N-(4-{[3-cyano-1-(2-methylpropyl)-1H-indol-5-yl]amino}phenyl)pyrrolidine-2-carboxamide, (2S)—N-(4-{[3-cyano-1-(2-methylpropyl)-1H-indol-5-yl](methyl)amino}phenyl)pyrrolidine-2-carboxamide, (2S)—N-(4-{[3-cyano-1-(2-methylpropyl)-1H-indol-5-yl]sulfanyl}phenyl)pyrrolidine-2-carboxamide, (2S)—N-(4-{[3-cyano-1-(2-methylpropyl)-1H-indol-5-yl]sulfonyl}phenyl)pyrrolidine-2-carboxamide, (2S)—N-(4-{[3-cyano-1-(2-methylpropyl)-1H-indol-5-yl]methyl}phenyl)pyrrolidine-2-carboxamide, (2S)—N-(4-{[3-cyano-1-(2-methylpropyl)-1H-indol-5-yl]oxy}phenyl)pyrrolidine-2-carboxamide, 2-amino-N-{4-[(2-amino-3-cyano-1-ethyl-1H-indol-5-yl)oxy]phenyl}acetamide, 2-amino-N-{4-[(2-amino-3-cyano-1-methyl-1H-indol-5-yl)oxy]phenyl}acetamide, (2S)-2-amino-N-{4-[(2-amino-3-cyano-1H-indol-5-yl)oxy]phenyl}-3-hydroxypropanamide, 2-amino-N-{4-[(2-amino-3-cyano-1H-indol-5-yl)oxy]phenyl}acetamide, 2-amino-N-(4-{[2-amino-3-cyano-1-(2-methylpropyl)-1H-indol-5-yl]oxy}phenyl)acetamide, 2-amino-N-{4-[(2-amino-1-benzyl-3-cyano-1H-indol-5-yl)oxy]phenyl}acetamide, 2-{2-amino-5-[4-(2-aminoacetamido)phenoxy]-3-cyano-1H-indol-1-yl}-N,N-dimethylacetamide, 2-amino-N-{4-[(3-cyano-1H-indol-5-yl)oxy]phenyl}acetamide, 2-amino-N-{4-[(3-cyano-1-ethyl-1H-indol-5-yl)oxy]phenyl}acetamide, N-{4-[(3-cyano-1-ethyl-1H-indol-5-yl)oxy]phenyl}-2-(methylamino)acetamide, N-{4-[(3-cyano-1-ethyl-1H-indol-5-yl)oxy]phenyl}-2-(dimethylamino)acetamide, (2S)—N-{4-[(3-cyano-1-ethyl-1H-indol-5-yl)oxy]phenyl}pyrrolidine-2-carboxamide, (2R)—N-{4-[(3-cyano-1-ethyl-1H-indol-5-yl)oxy]phenyl}pyrrolidine-2-carboxamide, (2S)—N-{4-[(3-cyano-1-ethyl-1H-indol-5-yl)oxy]phenyl}-N-methylpyrrolidine-2-carboxamide, (2S)—N-{4-[(3-cyano-1-ethyl-1H-indol-5-yl)oxy]phenyl}azetidine-2-carboxamide, (2S)—N-{4-[(3-cyano-1-ethyl-1H-indol-5-yl)oxy]phenyl}pyrrolidine-2-carboxamide, (2R)—N-{4-[(3-cyano-1-ethyl-1H-indol-5-yl)oxy]phenyl}pyrrolidine-2-carboxamide, (2S)—N-{4-[(3-cyano-1-ethyl-1H-indol-5-yl)oxy]phenyl}-N-methylpyrrolidine-2-carboxamide, (2S)—N-{4-[(3-cyano-1-ethyl-1H-indol-5-yl)oxy]phenyl}azetidine-2-carboxamide, (2S)—N-{4-[(3-cyano-1-ethyl-1H-indol-5-yl)oxy]phenyl}piperidine-2-carboxamide, (2S)—N-{4-[(3-cyano-1-ethyl-1H-indol-5-yl)oxy]phenyl}-N-methyl-5-oxopyrrolidine-2-carboxamide, (2S)—N-[4-({3-cyano-1-[(methylcarbamoyl)methyl]-1H-indol-5-yl}oxy)phenyl]pyrrolidine-2-carboxamide, (2S)—N-[4-({3-cyano-1-[2-(dimethylamino)ethyl]-1H-indol-5-yl}oxy)phenyl]pyrrolidine-2-carboxamide, (2S)—N-[4-({3-cyano-1-[(oxan-4-yl)methyl]-1H-indol-5-yl}oxy)phenyl]pyrrolidine-2-carboxamide, (2S)—N-{4-[(3-cyano-1-phenyl-1H-indol-5-yl)oxy]phenyl}pyrrolidine-2-carboxamide, (2S)—N-(4-{[3-cyano-1-(2-methyl-1-oxo-2,3-dihydro-1H-isoindol-5-yl)-1H-indol-5-yl]oxy}phenyl)pyrrolidine-2-carboxamide, (2S)—N-{4-[(1-benzyl-3-cyano-1H-indol-5-yl)oxy]phenyl}pyrrolidine-2-carboxamide, (2S)—N-[4-({3-cyano-1-[(3,5-dimethyl-1,2-oxazol-4-yl)methyl]-1H-indol-5-yl}oxy)phenyl]pyrrolidine-2-carboxamide, (2S)—N-(4-{[3-cyano-1-(2-methylpropyl)-1H-indazol-5-yl]oxy}phenyl)pyrrolidine-2-carboxamide, (2S)—N-[4-({3-cyano-1-[(3,5-dimethyl-1,2-oxazol-4-yl)methyl]-1H-indazol-5-yl}oxy)phenyl]pyrrolidine-2-carboxamide, 2-amino-N-(4-{[3-(1-methyl-1H-pyrazol-4-yl)-1H-indazol-5-yl]oxy}phenyl)acetamide, (2S)—N-(4-{[3-(1-methyl-1H-pyrazol-4-yl)-1H-indol-5-yl]oxy}phenyl)pyrrolidine-2-carboxamide, N-(4-{[3-cyano-1-(2-methylpropyl)-1H-indol-5-yl]oxy}phenyl)pyrrolidine-2-carboxamide.

343. PFKFB3 inhibitor for use in neuroprotection, wherein a PFKFB3 inhibitor is of formula (IX)

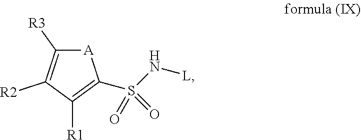

wherein: (i) A is O or S; and

R¹ is selected from H; halogen; and C1-C6 alkyl optionally substituted with at least one halogen; or

R¹ and R² form, together with the carbon atoms to which they are attached, a benzene ring optionally substituted with at least one R⁶; or a 5- or 6-membered heteroaromatic or heterocyclic ring, optionally substituted with at least one R⁶;

R² and R³ are independently selected from H; halogen; C1-C6 alkyl optionally substituted with at least one halogen; phenyl optionally substituted with at least one R⁶; 5- or 6-membered heteroaryl optionally substituted with at least one R⁶; and 5- or 6-membered arylsulfonyl or heteroaryl sulfonyl, optionally substituted with at least one R⁶; provided that
at least one of R² and R³ is selected from said phenyl, heteroaryl, arylsulfonyl and heteroarylsulfonyl, and
when L is (a), neither R² nor R³ is unsubstituted phenyl; or

R² and R³ form, together with the carbon atoms to which they are attached, a benzene ring optionally substituted with at least one R⁶; or a 5- or 6-membered heteroaromatic or hetero-cyclic ring, optionally substituted with at least one R⁶; or

(ii) A is CR′═CR′;

each R′ is independently selected from H; halogen; and C1-C6 alkyl optionally substituted with at least one halogen;

R¹ is selected from H; halogen; and C1-C6 alkyl optionally substituted with at least one halogen; or

R¹ and R² form, together with the carbon atoms to which they are attached, a benzene ring optionally substituted with at least one R⁶; or a 5- or 6-membered heteroaromatic or heterocyclic ring, optionally substituted with at least one R⁶;

R² and R³ are independently selected from H, halogen, C1-C6 alkyl optionally substituted with at least one halogen; phenyl optionally substituted with at least one R⁶; 5- or 6-membered heteroaryl optionally substituted with at least one R⁶; and 5- or 6-membered aryl-sulfonyl or heteroaryl sulfonyl, optionally substituted with at least one R⁶; provided that:
when both R² and R³ are selected from H, halogen and C1-C6 alkyl optionally substituted with at least one halogen, the ring containing A is substituted in ortho position relative to the sulphonamide bond with at least one substituent selected from halogen and C1-C6 alkyl optionally substituted with at least one halogen;
when L is (a), neither R² nor R³ is unsubstituted phenyl; and
when L is (c), R³ is optionally substituted phenyl only when R⁵ is tetrazol-5-yl, and R² is unsubstituted phenyl only when R⁴ is not hydroxy; or

R² and R³ form, together with the carbon atoms to which they are attached, a benzene ring optionally substituted with at least one R⁶, provided that said benzene ring is unsubstituted only when R⁵ is tetrazolyl or oxazolyl; or a 5- or 6-membered heteroaromatic or heterocyclic ring, optionally substituted with at least one R⁶; L is

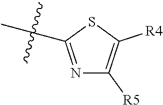

wherein R⁴ is COOR¹²; and R⁵ is selected from H and C1-C6 alkyl; or

R⁴ is selected from H and C1-C6 alkyl; and R⁵ is COOR¹²; or

(b)

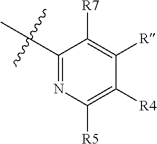

wherein R⁴ is selected from H and C1-C6 alkyl; R⁵ is selected from H and C1-C6 alkyl; and

R″ is selected from C0-C1 alkyl-COOR¹²; or

R⁵ is selected from COOR¹²; and R″ is selected from H and C1-C6 alkyl; and

R is selected from H, C1-C6 alkyl, and nitro;

(c)

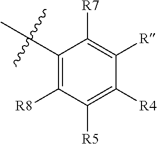

wherein R⁴ is selected from H, hydroxy and C1-C6 alkyl; R⁵ is selected from H, C1-C6 alkyl; and R″ is selected from C0-C1 alkyl-COOR¹²; or

R⁵ is selected from COOR¹², oxazol-5-yl and tetrazol-5-yl, said oxazol-5-yl and tetrazol-5-yl optionally being substituted by R⁹; and R″ is selected from H, C1-C6 alkyl, and nitro;

R⁷ is selected from H, C1-C6 alkyl, and nitro; and

R is selected from H, hydroxy, and C1-C6 alkyl; or

(d)

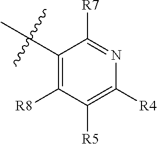

wherein R⁴ is selected from H and C1-C6 alkyl; and R⁵ is COOR¹²; R⁷ is selected from H, C1-C6 alkyl, and nitro; and R⁸ is selected from H, hydroxy, and C1-C6 alkyl;

provided that in any of (a), (b), (c) and (d), R⁴, R⁵ and R″ are selected from C0-C1 alkyl-COOR¹² only when at least one of R² or R³ is optionally substituted phenyl or optionally substituted heteroaryl; or when R² and R³ together with the carbon atoms to which they are attached form a benzene ring optionally substituted by at least one R⁶;

R⁶ is selected from C1-C6 alkyl, cyano, halogen, hydroxy, C1-C6 alkoxy, C1-C6 alkylthio, tetrahydropyrrolyl, R¹⁰R¹¹N, carbamoyl, and C1-C6 alkylcarbonylamino, or is an ethyleneoxy biradical forming, together with the atoms to which it is attached, a five-membered oxygen containing cycle; wherein any alkyl is optionally substituted with at least one halogen;

R⁹ is selected from C0-C1 alkyl-COOR¹²;

R¹⁰ and R¹¹ are independently selected from H and C1-C6 alkyl or form, together with the nitrogen to which they are attached, a 5- or 6-membered cyclic amino optionally containing one other cyclic heteroatom;

R¹² is selected from H, C1-C6 alkyl; heteroaryl-C0-C2 alkyl; (C₁-C₃ alkoxy)ₚC₁-C₃ alkyl; aryl-C0-C2 alkyl; heterocyclyl-C0-C2 alkyl; and C1-C6 dialkylamino-C1-C6 alkyl, wherein any cyclic moiety is optionally substituted with C1-C6 alkyl;

p is 1 or 2;

or a pharmaceutically acceptable salt thereof;

provided that the compound is not:

ethyl 2-(benzofuran-2-sulfonamido)thiazole-4-carboxylate;

ethyl 2-(5-methylbenzo[b]thiophene-2-sulfonamido)thiazole-4-carboxylate;

ethyl 2-(benzo[b]thiophene-2-sulfonamido)thiazole-4-carboxylate;

ethyl 2-(6-acetamidonaphthalene-2-sulfonamido)-4-methylthiazole-5-carboxylate;

ethyl 2-(6-aminonaphthalene-2-sulfonamido)-4-methylthiazole-5-carboxylate;

methyl 6-(4′-cyano-[1,1′-biphenyl]-4-ylsulfonamido)picolinate;

2-(3-(benzo[b]thiophene-2-sulfonamido)phenyl)acetic acid;

methyl 2-(3-(benzo[b]thiophene-2-sulfonamido)phenyl)acetate;

ethyl 3-(5-(6-oxo-1,6-dihydropyridazin-3-yl)furan-2-sulfonamido)benzoate;

ethyl 3-(5-(5-(trifluoromethyl)isoxazol-3-yl)furan-2-sulfonamido)benzoate;

ethyl 3-(5-(4,5-dimethyl-1H-pyrazol-3-yl)thiophene-2-sulfonamido)benzoate;

ethyl 3-(5-(5-methyl-1H-pyrazol-3-yl)thiophene-2-sulfonamido)benzoate;

ethyl 3-(5-(5-(trifluoromethyl)isoxazol-3-yl)thiophene-2-sulfonamido)benzoate;

ethyl 3-(5-(3-(trifluoromethyl)isoxazol-5-yl)thiophene-2-sulfonamido)benzoate;

ethyl 3-(5-(3-methylisoxazol-5-yl)thiophene-2-sulfonamido)benzoate;

ethyl 3-(4-(4-(tert-butyl)thiazol-2-yl)thiophene-2-sulfonamido)benzoate;

methyl 3-(4-(4-(tert-butyl)thiazol-2-yl)thiophene-2-sulfonamido)benzoate;

methyl 3-(3-(1H-tetrazol-1-yl)phenylsulfonamido)benzoate;

ethyl 3-(2-ethyl-5-(5-(trifluoromethyl)isoxazol-3-yl)phenylsulfonamido)benzoate;

ethyl 3-(2-methyl-5-(5-methyl-1,2,4-oxadiazol-3-yl)phenylsulfonamido)benzoate;

ethyl 3-(3-(5-methyl-1,2,4-oxadiazol-3-yl)phenylsulfonamido)benzoate;

ethyl 3-(2-methyl-5-(5-methyl-1H-pyrazol-3-yl)phenylsulfonamido)benzoate;

ethyl 3-(2-methyl-5-(2-methylthiazol-4-yl)phenylsulfonamido)benzoate;

ethyl 3-(2-isopropyl-5-(3-methylisoxazol-5-yl)phenylsulfonamido)benzoate;

ethyl 3-(2-methyl-5-(2-methyloxazol-5-yl)phenylsulfonamido)benzoate;

ethyl 3-(2-ethyl-5-(3-methylisoxazol-5-yl)phenylsulfonamido)benzoate;

ethyl 3-(4-(2-methyloxazol-4-yl)phenylsulfonamido)benzoate;

ethyl 3-(4-(2-methyloxazol-5-yl)phenylsulfonamido)benzoate;

methyl 3-(4-(2,5-dimethyloxazol-4-yl)phenylsulfonamido)benzoate;

ethyl 3-(2-methyl-5-(6-oxo-1,6-dihydropyridazin-3-yl)phenylsulfonamido)benzoate;

3-(6-butoxynaphthalene-2-sulfonamido)benzoic acid;

3-(6-methoxynaphthalene-2-sulfonamido)benzoic acid;

3-(6-propoxynaphthalene-2-sulfonamido)benzoic acid;

3-(6-methylnaphthalene-2-sulfonamido)benzoic acid;

3-(4-(3,5-dimethyl-1H-pyrazol-1-yl)phenylsulfonamido)benzoic acid;

N-(3-(2H-tetrazol-5-yl)phenyl)benzo[c][1,2,5]thiadiazole-4-sulfonamide;

N-(3-(2H-tetrazol-5-yl)phenyl)-2,3,5,6-tetramethylbenzenesulfonamide;

N-(3-(2H-tetrazol-5-yl)phenyl)-2,4,5-trichlorobenzenesulfonamide;

N-(3-(2H-tetrazol-5-yl)phenyl)-5-(tert-butyl)-2-methylbenzenesulfonamide;

3-methyl-N-(3-(oxazol-5-yl)phenyl)quinoline-8-sulfonamide;

5-bromo-2-methyl-N-(3-(oxazol-5-yl)phenyl)benzenesulfonamide;

2,5-dichloro-3,6-dimethyl-N-(3-(oxazol-5-yl)phenyl)benzenesulfonamide;

N-(3-(oxazol-5-yl)phenyl)-2,3-dihydrobenzofuran-5-sulfonamide;

2-chloro-4-methyl-N-(3-(oxazol-5-yl)phenyl)benzenesulfonamide;

2-chloro-4-fluoro-N-(3-(oxazol-5-yl)phenyl)benzenesulfonamide;

2-fluoro-N-(3-(oxazol-5-yl)phenyl)benzenesulfonamide;

N-(3-(oxazol-5-yl)phenyl)quinoline-8-sulfonamide;

N-(3-(oxazol-5-yl)phenyl)naphthalene-2-sulfonamide;

2-bromo-N-(3-(oxazol-5-yl)phenyl)benzenesulfonamide;

5-(dimethylamino)-N-(3-(oxazol-5-yl)phenyl)naphthalene-1-sulfonamide;

2,3,5, 6-tetramethyl-N-(3-(oxazol-5-yl)phenyl)benzenesulfonamide;

2,5-dichloro-N-(3-(oxazol-5-yl)phenyl)benzenesulfonamide; or

2,3,4-trifluoro-N-(3-(oxazol-5-yl)phenyl)benzenesulfonamide.

344. PFKFB3 inhibitor for use in neuroprotection, wherein a PFKFB3 inhibitor is of formula (IX) wherein:

(i) A is O or S; and

R¹ is selected from H; halogen; and C1-C6 alkyl optionally substituted with at least one halogen; or

R¹ and R² form, together with the carbon atoms to which they are attached, a benzene ring optionally substituted with at least one R⁶; or a 5- or 6-membered heteroaromatic or heterocyclic ring, optionally substituted with at least one R⁶;

R² and R³ are independently selected from H; halogen; C1-C6 alkyl optionally substituted with at least one halogen; phenyl optionally substituted with at least one R⁶; 5- or 6-membered heteroaryl optionally substituted with at least one R⁶; and 5- or 6-membered arylsulfonyl or heteroaryl sulfonyl, optionally substituted with at least one R⁶; provided that
at least one of R² and R³ is selected from said phenyl, heteroaryl, arylsulfonyl and heteroarylsulfonyl, and
when L is (a), neither R² nor R³ is unsubstituted phenyl; or

R² and R³ form, together with the carbon atoms to which they are attached, a benzene ring optionally substituted with at least one R⁶; or a 5- or 6-membered heteroaromatic or hetero-cyclic ring, optionally substituted with at least one R⁶; or

(ii) A is CR′═CR′;

each R′ is independently selected from H; halogen; and C1-C6 alkyl optionally substituted with at least one halogen;

R¹ is selected from H; halogen; and C1-C6 alkyl optionally substituted with at least one halogen; or

R¹ and R² form, together with the carbon atoms to which they are attached, a benzene ring optionally substituted with at least one R⁶; or a 5- or 6-membered heteroaromatic or heterocyclic ring, optionally substituted with at least one R⁶;

R² and R³ are independently selected from H, halogen, C1-C6 alkyl optionally substituted with at least one halogen; phenyl optionally substituted with at least one R⁶; 5- or 6-membered heteroaryl optionally substituted with at least one R⁶; and 5- or 6-membered aryl-sulfonyl or heteroaryl sulfonyl, optionally substituted with at least one R⁶; provided that:
when both R² and R³ are selected from H, halogen and C1-C6 alkyl optionally substituted with at least one halogen, the ring containing A is substituted in ortho position relative to the sulphonamide bond with at least one substituent selected from halogen and C1-C6 alkyl optionally substituted with at least one halogen;
when L is (a), neither R² nor R³ is unsubstituted phenyl; and
when L is (c), R³ is optionally substituted phenyl only when R⁵ is tetrazol-5-yl, and R² is unsubstituted phenyl only when R⁴ is not hydroxy; or

R² and R³ form, together with the carbon atoms to which they are attached, a benzene ring optionally substituted with at least one R⁶, provided that said benzene ring is unsubstituted only when R⁵ is tetrazolyl or oxazolyl; or a 5- or 6-membered heteroaromatic or heterocyclic ring, optionally substituted with at least one R⁶;

L is

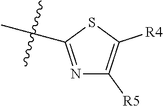

wherein R⁴ is COOR¹²; and R⁵ is selected from H and C1-C6 alkyl; or

R⁴ is selected from H and C1-C6 alkyl; and R⁵ is COOR¹²; or

(b)

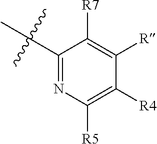

wherein R⁴ is selected from H and C1-C6 alkyl; R⁵ is selected from H and C1-C6 alkyl; and R″ is selected from C0-C1 alkyl-COOR¹²; or

R⁵ is selected from COOR¹²; and R″ is selected from H and C1-C6 alkyl; and

R is selected from H, C1-C6 alkyl, and nitro;

(c)

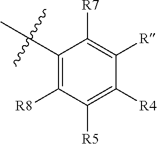

wherein R⁴ is selected from H, hydroxy and C1-C6 alkyl; R⁵ is selected from H, C1-C6 alkyl; and R″ is selected from C0-C1 alkyl-COOR¹²; or

R⁵ is selected from COOR¹², oxazol-5-yl and tetrazol-5-yl, said oxazol-5-yl and tetrazol-5-yl optionally being substituted by R⁹; and R″ is selected from H, C1-C6 alkyl, and nitro;

R⁷ is selected from H, C1-C6 alkyl, and nitro; and

R is selected from H, hydroxy, and C1-C6 alkyl; or

(d)

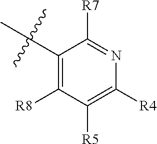

wherein R⁴ is selected from H and C1-C6 alkyl; and R⁵ is COOR¹²; R⁷ is selected from H, C1-C6 alkyl, and nitro; and R⁸ is selected from H, hydroxy, and C1-C6 alkyl;

provided that in any of (a), (b), (c) and (d), R⁴, R⁵ and R″ are selected from C0-C1 alkyl-COOR¹² only when at least one of R² or R³ is optionally substituted phenyl or optionally substituted heteroaryl; or when R² and R³ together with the carbon atoms to which they are attached form a benzene ring optionally substituted by at least one R⁶;

R⁶ is selected from C1-C6 alkyl, cyano, halogen, hydroxy, C1-C6 alkoxy, C1-C6 alkylthio, tetrahydropyrrolyl, R¹⁰R¹¹N, carbamoyl, and C1-C6 alkylcarbonylamino, or is an ethyleneoxy biradical forming, together with the atoms to which it is attached, a five-membered oxygen containing cycle; wherein any alkyl is optionally substituted with at least one halogen;

R⁹ is selected from C0-C1 alkyl-COOR¹²;

R¹⁰ and R¹¹ are independently selected from H and C1-C6 alkyl or form, together with the nitrogen to which they are attached, a 5- or 6-membered cyclic amino optionally containing one other cyclic heteroatom;

R¹² is selected from H, C1-C6 alkyl; heteroaryl-C0-C2 alkyl; (C1-C3 alkoxy)ₚC1-C3 alkyl; aryl-C0-C2 alkyl; heterocyclyl-C0-C2 alkyl; and C1-C6 dialkylamino-C1-C6 alkyl, wherein any cyclic moiety is optionally substituted with C1-C6 alkyl;

p is 1 or 2; or a pharmaceutically acceptable salt thereof;

345. PFKFB3 inhibitor for use in neuroprotection, wherein a PFKFB3 inhibitor is of formula (X)

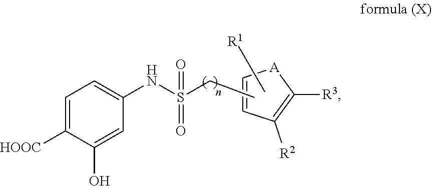

wherein: n is 0 or 1;

A is O, S, —CR⁴═CR⁴— or —CR⁴═N—;

R¹ is selected from H; halogen; C₁-C₆ alkyl, optionally substituted with at least one halogen; and C₁-C₆ alkoxy, substituted with at least one halogen;

R² and R³ are each independently selected from H; halogen; C₁-C₆ alkyl; C₁-C₆ alkoxy; secondary or tertiary C₁-C₆ alkylamido; carbocyclylcarbonylamino-C₁-C₂ alkyl; 5- or 6-membered cyclic aminocarbonyl; C₁-C₆ alkylcarbonylamino; C₁-C₆ alkylsulfonyl; hydroxy-C₀-C₆ alkyl, C₁-C₆ alkylcarbonyl; carboxy; C₁-C₆ alkoxycarbonyl; cyano; nitro; carbocyclyloxy; heterocyclyloxy; carbocyclyl-C₀-C₃ alkyl; carbocyclyl-C₂-C₃ alkenyl; heterocyclyl-C₀-C₃ alkyl; and heterocyclyl-C₂-C₃ alkenyl;

wherein any alkyl is optionally substituted with at least one halogen; any carbocyclyl or heterocyclyl is 5- or 6-membered monocyclyl or 9- or 10-membered bicyclyl; and any carbocyclyl or heterocyclyl is optionally substituted with at least one R⁵; or R² and R³ form, together with the carbon atoms to which they are attached, a 5- or 6-membered carbocyclic or heterocyclic ring, which ring is optionally substituted with at least one R⁵;

each R⁴ is independently selected from H, halogen, monocyclic C₃-C₆ carbocyclyl and C₁-C₆ alkyl, wherein any alkyl is optionally substituted with at least one halogen;

each R⁵ is independently selected from halogen; C₁-C₆ alkyl; C₁-C₆ alkoxy; phenoxy; amino; cyano; nitro; secondary or tertiary C₁-C₆ alkylamino; 5- or 6-membered cyclic amino; C₁-C₆ alkylcarbonylamino; carbamoyl; secondary or tertiary C₁-C₆ alkylamido; 5- or 6-membered cyclic aminocarbonyl; C₁-C₆ alkoxycarbonylamino; hydroxy-C₀-C₆ alkyl; C₁-C₆-alkylthio; carboxy-C₀-C₆-alkyl; C₁-C₆ alkoxycarbonyl; C₁-C₆ alkylcarbonyl; C₁-C₆-alkylsulfonyl; and C₁-C₆ alkylsulfonylamino; wherein any alkyl is optionally substituted with at least one halogen; or a pharmaceutically acceptable salt thereof, for use as a medicament, with the proviso that when A is CR⁴—CR⁴ and n is 0, then neither R² nor R³ is selected from 4-hydroxypyrazolo[1,5-a]-1,3,5-triazin-8-yl and 2,4-dihydroxypyrazolo[1,5-a]-1,3,5-triazin-8-yl; the compound is not selected from

4-(3,4-dichlorophenylsulfonamido)-2-hydroxybenzoic acid,

4-(2,5-dichlorophenylsulfonamido)-2-hydroxybenzoic acid,

4-(2,5-diethylphenylsulfonamido)-2-hydroxybenzoic acid,

4-(4-bromophenylsulfonamido)-2-hydroxybenzoic acid,

4-(3-carboxy-4-hydroxyphenylsulfonamido)-2-hydroxybenzoic acid,

2-hydroxy-4-(4-methylphenylsulfonamido)benzoic acid,

2-hydroxy-4-(phenylsulfonamido)benzoic acid, and

4-(4-ethylphenylsulfonamido)-2-hydroxybenzoic acid.

346. PFKFB3 inhibitor for use in neuroprotection, wherein a PFKFB3 inhibitor is of formula (X) wherein:

n is 0 or 1;

A is O, S, —CR⁴═CR⁴— or —CR⁴═N—;

R¹ is selected from H; halogen; C₁-C₆ alkyl, optionally substituted with at least one halogen; and C₁-C₆ alkoxy, substituted with at least one halogen;

R² and R³ are each independently selected from H; halogen; C₁-C₆ alkyl; C₁-C₆ alkoxy; secondary or tertiary C₁-C₆ alkylamido; carbocyclylcarbonylamino-C₁-C₂ alkyl; 5- or 6-membered cyclic aminocarbonyl; C₁-C₆ alkylcarbonylamino; C₁-C₆ alkylsulfonyl; hydroxy-C₀-C₆ alkyl, C₁-C₆ alkylcarbonyl; carboxy; C₁-C₆ alkoxycarbonyl; cyano; nitro; carbocyclyloxy; heterocyclyloxy; carbocyclyl-C₀-C₃ alkyl; carbocyclyl-C₂-C₃ alkenyl; heterocyclyl-C₀-C₃ alkyl; and heterocyclyl-C₂-C₃ alkenyl;

wherein any alkyl is optionally substituted with at least one halogen; any carbocyclyl or heterocyclyl is 5- or 6-membered monocyclyl or 9- or 10-membered bicyclyl; and any carbocyclyl or heterocyclyl is optionally substituted with at least one R⁵; or R² and R³ form, together with the carbon atoms to which they are attached, a 5- or 6-membered carbocyclic or heterocyclic ring, which ring is optionally substituted with at least one R⁵;

each R⁴ is independently selected from H, halogen, monocyclic C₃-C₆ carbocyclyl and C₁-C₆ alkyl, wherein any alkyl is optionally substituted with at least one halogen;

each R⁵ is independently selected from halogen; C₁-C₆ alkyl; C₁-C₆ alkoxy; phenoxy; amino; cyano; nitro; secondary or tertiary C₁-C₆ alkylamino; 5- or 6-membered cyclic amino; C₁-C₆ alkylcarbonylamino; carbamoyl; secondary or tertiary C₁-C₆ alkylamido; 5- or 6-membered cyclic aminocarbonyl; C₁-C₆ alkoxycarbonylamino; hydroxy-C₀-C₆ alkyl; C₁-C₆-alkylthio; carboxy-C₀-C₆-alkyl; C₁-C₆ alkoxycarbonyl; C₁-C₆ alkylcarbonyl; C₁-C₆-alkylsulfonyl; and C₁-C₆ alkylsulfonylamino; wherein any alkyl is optionally substituted with at least one halogen; or a pharmaceutically acceptable salt thereof, for use as a medicament, with the proviso that when A is CR⁴═CR⁴ and n is 0, then neither R² nor R³ is selected from 4-hydroxypyrazolo[1,5-a]-1,3,5-triazin-8-yl and 2,4-dihydroxypyrazolo[1,5-a]-1,3,5-triazin-8-yl;

347. PFKFB3 inhibitor for use in neuroprotection, wherein a PFKFB3 inhibitor is of formula of formula (XI)

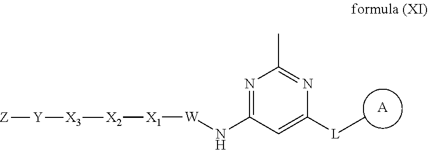

or a pharmaceutically acceptable salt thereof, wherein: W is a branched or straight C₁₋₁₂ aliphatic chain wherein up to two carbon units are optionally and independently replaced by —C(Q₁)₂-, —C(Q₂)₂-, -CHQ₁-, -CHQ₂-, —CO—, —CS—, —CONR^{A}—, —CONR^{A}NR^{A}—, —CO₂—, —OCO—, —NR^{A}—, —NR^{A}CO₂—, —O—, —NR^{A}CONR^{A}—, —OCONR^{A}—, —NR^{A}NR^{A}—, —NR^{A}CO—, —S—, —SO—, —SO₂—, —SO₂NR^{A}., —NR^{A}SO₂—, or —NR^{A}SO₂NR^{A};

each R^{A} is independently hydrogen C₁₋₈ aliphatic; cycloaliphatic, heterocycloaliphatic, aryl, or heteroaryl, optionally substituted with 1-3 of Q₁ or Q₂;

X₁, X², and X₃ are each independently absent or are a cycloaliphatic, heterocycloaliphatic, aryl, or heteroaryl, each or which are optionally and independently substituted with 1-3 of Q₁, or Q₂, and wherein at least one of X₁, X² and X₃ is present;

Y is absent or is a branched or straight C₁₋₁₂ aliphatic chain wherein up to two carbon units are optionally and independently replaced by —C(Q₁)₂-, —C(Q₂)₂-, -CHQ₁-, -CHQ₂-, —CO—, —CS—, —CONR^{B}—, —C(═NR^{B})NR^{B}—, —C(═NOR^{B})NR^{B}—, —NR^{B}C(═NR^{B})NR^{B}—, —CONR^{B}NR^{B}—,—CO₂—, —OCO—, —NR—, —NR^{B}CO₂—, —O—, —NR^{B}CONR^{B}—, —OCONR^{B}—, —NR^{B}NR^{B}—, —NR^{B}CO—, —S—, —SO—, —SO₂—, —SO₂NR^{B}—, —NRSO₂—, or —NR^{B}SO₂NR;

each R^{B} is independently hydrogen, C₁₋₈ aliphatic, cycloaliphatic, heterocycloaliphatic, aryl, or heteroaryl, optionally substituted with 1-3 of Q₁ or Q₂;

Z is independently hydrogen, C₁₋₈ aliphatic, cycloaliphatic, heterocycloaliphatic, aryl, or heteroaryl, optionally substituted with 1-3 of Q₁ or Q₂; or

L is absent or is NH, N(Cl₋ₑ aliphatic), or is a branched or straight C aliphatic chain wherein up to two carbon units of L are optionally and independently replaced by —C(Q₁)₂-, —C(Q₂)₂-, —CO—, —CS—, —CONR_{C}—, —CONR^{C}NR^{C}—, —CO₂—, —OCO—, —NR^{C}—, —NR^{C}CO₂—, —O—, —NR^{C}CONR^{C}—, —OCONR^{C}—, —NR^{C}NR^{C}—, —NR^{C}CO—, —S—, —SO—, —SO₂—, —SO₂NR^{C}—, —NR^{C}SO₂—, or —NR^{C}SO₂NR^{C};

each R_{C} is independently hydrogen, C₁₋₈ aliphatic, cycloaliphatic, heterocycloaliphatic, aryl, or heteroaryl, optionally substituted with 1-3 of Q₁ or Q₂;

Ring A is a monocyclic, bicyclic, or tricyclic cycloaliphatic, heterocycloaliphatic, aryl, or heteroaryl, any of which may be optionally substituted with 1-3 of halo, —OH, oxo, —CF₃, —OCF₃, cyano, or a C₁₋₈ branched or straight aliphatic, wherein 1-3 methylene groups of the aliphatic are optionally and independently replaced with —C(O)—, —O—, —NH—, —C(O)NH—, or —C(O)O—, and wherein the aliphatic is optionally further substituted with 1-3 of halo, cyano, OH or C₁₋₃ aliphatic;

each Q, is independently halo, oxo, —CN, —NO₂, —N═O, —NHOQ₂, =NQ₂, =NOQ₂, —OQ₂, —SOQ₂, —SO₂Q₂, —SON(Q₂)₂, —SO₂(Q₂)₂, —N(Q₂)₂, —C(O)OQ₂, —C(O)-Q₂, —C(O)N(Q₂)₂, —C(═NQ₂)NQ₂-, —NQ₂C(═NQ₂)NQ₂-, —C(O)N(Q₂)(OQ₂), —N(Q₂)C(O)-Q₂, —N(Q₂)C(O)N(Q₂)₂, —N(Q₂)C(O)O-Q₂, —N(Q₂)SO₂-Q₂, —N(Q₂)SO-Q₂ or aliphatic optionally including 1-3 substituents independently selected from Q₂ or Q₃,

each Q₂ is independently hydrogen, aliphatic, alkoxy, cycloaliphatic, aryl, arylalkyl, heterocyclic, or heteroaryl ring, each optionally including 1-3 substituents independently selected from Q₃;

each Q₃ is halo, oxo, CN, NO₂, NH₂, CF₃OCF₃, OH, —COOH, or C₁-C₄ alkyl optionally substituted with 1-3 of halo, oxo, —CN, —NO₂, —CF₃, —OCF₃, —OH, —SH, —S(O)₃H,—NH₂, or —COOH;

provided that the compound of formula XI is not

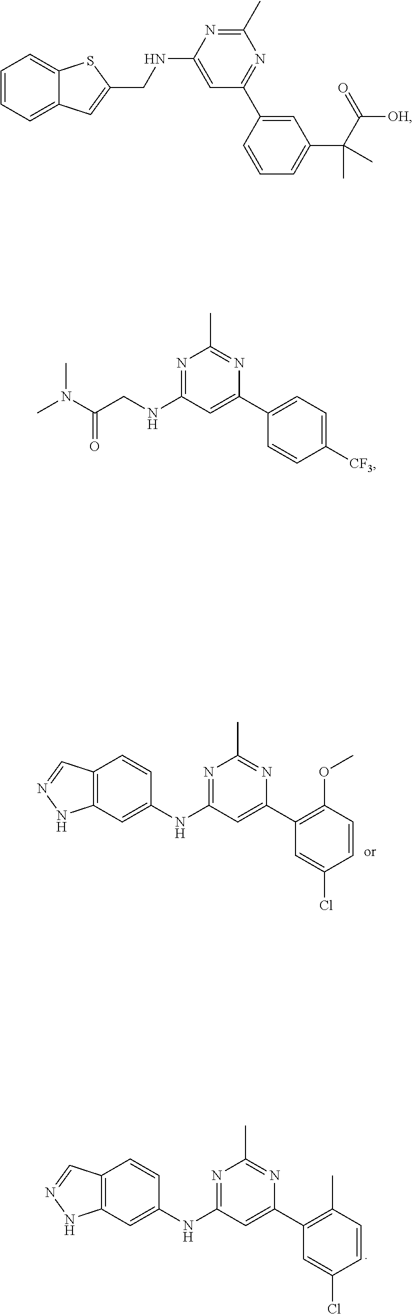 [SMILES: Cc1nc(NCc2cc3ccccc3s2)cc(-c2cccc(C(C)(C)C(=O)O)c2)n1 ; Cc1nc(NCC(=O)N(C)C)cc(-c2ccc(C(F)(F)F)cc2)n1 ; COc1ccc(Cl)cc1-c1cc(Nc2ccc3cn[nH]c3c2)nc(C)n1 ; Cc1nc(Nc2ccc3cn[nH]c3c2)cc(-c2cc(Cl)ccc2C)n1]

348. PFKFB3 inhibitor for use in neuroprotection, wherein a PFKFB3 inhibitor is of formula (XI) or a pharmaceutically acceptable salt thereof, wherein:

W is a branched or straight C₁₋₁₂ aliphatic chain wherein up to two carbon units are optionally and independently replaced by —C(Q₁)₂-, —C(Q₂)₂-, -CHQ₁-, -CHQ₂-, —CO—, —CS—, —CONR^{A}—, —CONR^{A}NR^{A}—, —CO₂—, —OCO—, —NR^{A}., —NR^{A}CO₂—, —O—, —NR^{A}CONR^{A}—, —OCONR^{A}—, —NR^{A}NR^{A}—, —NR^{A}CO—, —S—, —SO—, —SO₂—, —SO₂NR^{A}—, —NR^{A}SO₂—, or —NR^{A}SO₂NR^{A};
each R^{A} is independently hydrogen, C₁₋₈ aliphatic, cycloaliphatic, heterocycloaliphatic, aryl, or heteroaryl, optionally substituted with 1-3 of Q₁ or Q₂;
X₁, X², and X₃ are each independently absent or are a cycloaliphatic, heterocycloaliphatic, aryl, or heteroaryl, each or which are optionally and independently substituted with 1-3 of Q₁, or Q₂, and wherein at least one of X₁, X² and X₃ is present;
Y is absent or is a branched or straight C₁₋₁₂ aliphatic chain wherein up to two carbon units are optionally and independently replaced by —C(Q₁)₂-, —C(Q₂)₂-, -CHQ₁-, -CHQ₂-, —CO—, —CS—, —CONR^{B}—, —C(═NR^{B})NR^{B}—, —C(═NOR^{B})NR^{B}—, —NR^{B}C(═NR^{B})NR^{B}—, —CONR^{B}NR^{B}—, —CO₂—, —OCO—, —NR—, —NR^{B}CO₂—, —O—, —NR^{B}CONR^{B}—, —OCONR^{B}—, —NR^{B}NR^{B}—, —NR^{B}CO—, —S—, —SO—, —SO₂—, —SO₂NR^{B}—, —NRSO₂—, or —NR^{B}SO₂NR;
each R^{B} is independently hydrogen, C₁₋₈ aliphatic, cycloaliphatic, heterocycloaliphatic, aryl, or heteroaryl, optionally substituted with 1-3 of Q₁ or Q₂;
Z is independently hydrogen, C₁₋₈ aliphatic, cycloaliphatic, heterocycloaliphatic, aryl, or heteroaryl, optionally substituted with 1-3 of Q₁ or Q₂; or
L is absent or is NH, N(Cl₋ₑ aliphatic), or is a branched or straight C aliphatic chain wherein up to two carbon units of L are optionally and independently replaced by —C(Q₁)₂-, —C(Q₂)₂-, —CO—, —CS—, —CONR_{C}—, —CONR^{C}NR^{C}—, —CO₂—, —OCO—, —NR^{C}—, —NR^{C}CO₂—, —O—, —NR^{C}CONR^{C}—, —OCONR^{C}—, —NR^{C}NR^{C}—, —NR^{C}CO—, —S—, —SO—, —SO₂—, —SO₂NR^{C}—, —NR^{C}SO₂—, or —NR^{C}SO₂NR^{C};
each R_{C} is independently hydrogen, C₁₋₈ aliphatic, cycloaliphatic, heterocycloaliphatic, aryl, or heteroaryl, optionally substituted with 1-3 of Q₁ or Q₂;
Ring A is a monocyclic, bicyclic, or tricyclic cycloaliphatic, heterocycloaliphatic, aryl, or heteroaryl, any of which may be optionally substituted with 1-3 of halo, —OH, oxo, —CF₃, —OCF₃, cyano, or a C₁₋₈ branched or straight aliphatic, wherein 1-3 methylene groups of the aliphatic are optionally and independently replaced with —C(O)—, —O—, —NH—, —C(O)NH—, or —C(O)O—, and wherein the aliphatic is optionally further substituted with 1-3 of halo, cyano, OH or C₁₋₃ aliphatic;
each Q, is independently halo, oxo, —CN, —NO₂, —N═O, —NHOQ₂, =NQ₂, =NOQ₂, —OQ₂, —SOQ₂, —SO₂Q₂, —SON(Q₂)₂, —SO₂ (Q₂)₂, —N(Q₂)₂, —C(O)OQ₂, —C(O)-Q₂, —C(O)N(Q₂)₂, —C(═NQ₂)NQ₂-, —NQ₂C(═NQ₂)NQ₂-, —C(O)N(Q₂)(OQ₂), —N(Q₂)C(O)-Q₂, —N(Q₂)C(O)N(Q₂)₂, —N(Q₂)C(O)O-Q₂, —N(Q₂)SO₂-Q₂-N(Q₂)SO-Q₂ or aliphatic optionally including 1-3 substituents independently selected from Q₂ or Q₃;
each Q₂ is independently hydrogen, aliphatic, alkoxy, cycloaliphatic, aryl, arylalkyl, heterocyclic, or heteroaryl ring, each optionally including 1-3 substituents independently selected from Q₃;
each Q₃ is halo, oxo, CN, NO₂, NH₂, CF₃OCF₃, OH, —COOH, or C₁-C₄ alkyl optionally substituted with 1-3 of halo, oxo, —CN, —NO₂, —CF₃, —OCF₃, —OH, —SH, —S(O)₃H, —NH₂, or —COOH;

349. PFKFB3 inhibitor for use in neuroprotection, wherein a PFKFB3 inhibitor is of formula (XII)

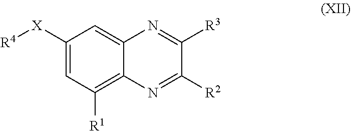

wherein
X denotes N—R⁵ or O;
R¹ denotes ArX, ArX—Ar^{Y}, Ar^{X}-Hetar^{Y}, Ar-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, CA^{X};
R² and R¹ denote independently from each other H, —OH, —SH, straight-chain or branched —C₁₋₆-alkyl, straight-chain or branched —C₂₋₆-alkenyl, straight-chain or branched —O—C₁₋₆-alkyl, straight-chain or branched —S—C₁₋₆-alkyl, Hal, —CN, —NH₂, —NH(C₁₋₄-alkyl), N(C₁₋₄-alkyl)₂,
wherein the C₁₋₄-alkyl substituents may be the same or different and may be straight-chain or branched;
R⁴ denotes Ar^{W} or Hetar^{w}; Ar^{W} or Hetar^{w} bears in its ortho-position (relative to the attachment of R⁴ to X) one (1) substituent R^{W1} and may or may not bear further substituents;
R⁵ denotes H, Ar^{X}, Hetar^{X}, Hetcyc^{X}, LA^{X}, CA^{X};
Ar^{W} denotes a mono-, bi- or tricyclic aromatic ring system with 5, 6, 7, 8, 9, 10, 11, 12, 13, 14 ring carbon atoms; the ring system may bear—besides the ortho-substituent R^{W1}—no further substituent or one (1) further substituent R^{W2} or two (2) further substituents R^{W2}, R^{W3}, that may be the same or different;
   Ar^{X} denotes a mono-, bi- or tricyclic aromatic ring system with 5, 6, 7, 8, 9, 10, 11, 12, 13, 14 ring carbon atoms, wherein the ring system may be unsubstituted or mono-, di- or trisubstituted with independently from each other R^{X1}, R^{X2}, R^{X3};
   Ar^{Y} denotes a mono-, bi- or tricyclic aromatic ring system with 5, 6, 7, 8, 9, 10, 11, 12, 13, 14 ring carbon atoms, wherein the ring system may be unsubstituted or mono-, di- or trisubstituted with independently from each other R^{Y1}, R^{Y2}, R^{Y3};
   Hetar^{W} denotes a mono-, bi- or tricyclic aromatic ring system with 5, 6, 7, 8, 9, 10, 11, 12, 13, 14 ring atoms, wherein 1, 2, 3, 4, 5 of said ring atoms is/are a hetero atom(s) selected from N, O and/or S, and the remaining are carbon atoms, wherein that ring system may bear—besides the ortho-substituent R^{W1}—no further substituent or one (1) further substituent R^{W2} or two (2) further substituents R^{W2}, R^{W3} that may be the same or different;
   Hetar^{X} denotes a mono-, bi- or tricyclic aromatic ring system with 5, 6, 7, 8, 9, 10, 11, 12, 13, 14 ring atoms, wherein 1, 2, 3, 4, 5 of said ring atoms is/are a hetero atom(s) selected from N, O and/or S, and the remaining are carbon atoms, wherein that aromatic ring system may be unsubstituted or mono-, di- or tri-substituted with independently from each other R^{X1}, R^{X2}, R^{X3};
   Hetar^{Y} denotes a mono-, bi- or tricyclic aromatic ring system with 5, 6, 7, 8, 9, 10, 11, 12, 13, 14 ring atoms, wherein 1, 2, 3, 4, 5 of said ring atoms is/are a hetero atom(s) selected from N, O and/or S, and the remaining are carbon atoms, wherein that aromatic ring system may be unsubstituted or mono-, di- or tri-substituted with independently from each other R^{Y1}, R^{Y2}, R^{Y3};
   Hetcyc^{X} denotes a saturated or partially unsaturated mono-, bi- or tricyclic heterocycle with 3, 4, 5, 6, 7, 8, 9, 10, 11, 12, 13, 14 ring atoms, wherein 1, 2, 3, 4, 5 ring atom(s) is/are heteroatom(s) selected from N, O and/or S, and the remaining ring atoms are carbon atoms, wherein that heterocycle may be unsubstituted or mono-, di- or trisubstituted with R^{X4}, R^{X5}, R^{X6};
   Hetcyc^{Y} denotes a saturated or partially unsaturated mono-, bi- or tricyclic heterocycle with 3, 4, 5, 6, 7, 8, 9, 10, 11, 12, 13, 14 ring atoms, wherein 1, 2, 3, 4, 5 ring atom(s) is/are heteroatom(s) selected from N, O and/or S, and the remaining ring atoms are carbon atoms, wherein that heterocycle may be unsubstituted or mono-, di- or trisubstituted with R^{Y4}, R^{Y5}, R^{Y6};
   R^{W1} denotes Hal, LA^{X}, CA^{X}, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{X}, Hetar^{X}-LA^{Z}-Ar^{Y}, Heta^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, —CN, —NO₂, —SO₂NH₂, —SO₂NHR^{W4}, —SO₂NR^{W4}R^{W5}, —NH—SO₂—R^{W6}, —NR^{W4}—SO₂—R^{W6}, —S—R^{W6}, —S(═O)—R^{W6}, —SO₂—R^{W6}, —NH₂, —NHR^{W4}, —NR^{W4}R^{W5}, —OH, —O—R^{W6}, —CHO, —C(═O)—R^{W6}, —COOH, —C(═O)—O—R^{W6}, —C(═O)—NH₂, —C(═O)—NHR^{W4}, —C(═O)—NR^{W4}R^{W5}, —NH—C(═O)—R^{W6}, —NR^{W4}—C(═O)—R^{W6}, —NH—(C₁₋₃-alkylene)-C(═O)—NH₂, —NH—(C₁₋₃-alkylene)-C(═O)—NHR^{W4}, —NH—(C₁₋₃-alkylene)-C(═O)—NR^{W4}R^{W5}, or
   R^{W1} and R⁵ form together a divalent alkylene chain with 1, 2, 3, 4, 5 chain carbon atoms, wherein 2 adjacent CH₂ groups may together be replaced by a —CH═CH— moiety; the divalent alkylene chain may be straight-chain or branched and may be unsubstituted or mono- or di-substituted with independently from each other straight-chain or branched —C₁₋₆-alkyl or ═O (oxo);
   R^{W2}, R^{W3} denote independently from each other H, Hal, LA^{X}, CA^{X}, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, —CN, —NO₂, —SO₂NH₂, —SO₂NHR^{W4}, —SO₂NR^{W4}R^{W5}, —NH—SO₂—R^{W6}, —NR^{W4}—SO₂—R^{W6}, S—R^{W6}, —S(═O)—R^{W6}, —SO₂—R^{W6}, —NH₂, —NHR^{W4}, —NR^{W4}R^{W5}, —NH—C(═O)—R^{W6}, —NR^{W4}—C(═O)—R^{W6}, —OH, —O—R^{W6}, —CHO, —C(═O)—R^{W6}, —COOH, —C(═O)—O—R^{W6}, —C(═O)—NH₂, —C(═O)—NHR^{W4}, —C(═O)—NR^{W4}R^{W5}, —C(═O)—NH—NH₂, —C(═O)—NH—NHR^{W4}, —NH—(C₁₋₃-alkylene)-C(═O)—NH₂, —NH—(C₁₋₃-alkylene)-C(═O)—NHR^{W4}, —NH—(C₁₋₃-alkylene)-C(═O)—NR^{W4}R^{W5},
   or
   two of R^{W1}, R^{W2} and R^{W3} form a divalent alkylene chain with 3, 4, 5 chain carbon atoms, wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, wherein the divalent alkylene chain may be unsubstituted or mono- or di-substituted with independently from each other straight-chain or branched —C₁₋₆-alkyl or ═O (oxo);
   R^{X1}, R^{X2}, R^{X3} denote independently from each other H, Hal, LA^{X}, CA^{X}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9},—NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}
   or
   two of R^{X1}, R^{X2}, R^{X3} form a divalent alkylene chain with 3, 4, 5 chain carbon atoms, wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, wherein the divalent alkylene chain may be unsubstituted or mono- or di-substituted with independently from each other straight-chain or branched —C₁₋₆-alkyl or ═O (oxo);
R^{X4}, R^{X5}, R^{X6} denote independently from each other H, Hal, LA^{X}, CA^{X}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{XB}, oxo (═O);
R^{Y1}, R^{Y2}, R^{Y3} denote independently from each other H, Hal, LAY, CAY, —CN, —NO₂, —SF₅, —SO₂NH₂, —SO₂NHR^{Y7}, —SO₂NR^{Y7}R^{Y8}, —NH—SO₂—R^{Y9}, —NR^{Y}—SO₂—R^{Y9}, —S—R^{Y9}, —S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y}—C(═O)—R^{Y9}, —NH—(C₁₋₃-alkylene)-C(═O)—NH₂, —NH—(C₁-3-alkylene)-C(═O)—NHR^{Y7}, —NH—(C₁₋₃-alkylene)-C(═O)—NR^{Y7}R^{Y8}
   or
two of R^{Y1}, R^{Y2}, R^{Y3} form a divalent alkylene chain with 3, 4, 5 chain carbon atoms, wherein 1 or 2 non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, wherein the divalent alkylene chain may be unsubstituted or mono- or di-substituted with independently from each other straight-chain or branched —C₁₋₆-alkyl or ═O (oxo);
R^{Y4}, R^{Y5}, R^{YS} denote independently from each other H, Hal, LAY, CAY, —CN, —NO₂, —SF₅, —SO₂NH₂, —SO₂NHR^{Y}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y}, —C(═O)—NR^{Y}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, or ═O (oxo);
LA^{X} denotes straight-chain or branched C₁₆-alkyl which may be unsubstituted or mono-, di- or trisubstituted with independently from each other Hal, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O), wherein 1 or 2 non-adjacent CH₂ groups of the C₁₋₆-alkyl radical may independently from each other be replaced by O, S, N(H) or N—R^{X7}, and/or 1 or 2 non-adjacent CH groups of the C₁₋₆-alkyl radical may independently from each other be replaced by N;
LA^{Y} denotes straight-chain or branched C₁₋₆-alkyl which may be unsubstituted or mono-, di- or trisubstituted with independently from each other Hal, —CN, —NO₂, —SF₅, —SO₂NH₂, —SO₂NHR^{Y7}, —SO₂NR^{Y}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y}R⁸, —OH, —O—R^{Y9}, —CHO, —C(═O)—R^{Y9}, —COOH, —C(═O)—O—R^{Y9}, —C(═O)—NH₂, —C(═O)—NHR^{Y}, —C(═O)—NR^{Y}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y}, —NH—(C₁₋₃-alkylene)-C(═O)—NR^{Y7}R^{Y8}, oxo (═O), wherein 1 or 2 non-adjacent CH₂ groups of the C₁₋₆-alkyl radical may independently from each other be replaced by O, S, N(H) or N—R^{Y7} and/or 1 or 2 non-adjacent CH groups of the C₁₋₆-alkyl radical may independently from each other be replaced by N;
LA^{Z} denotes a divalent straight-chain or branched C₁₋₆-alkylene radical, wherein the alkylene radical may be unsubstituted or mono-, di- or trisubstituted with independently from each other Hal, —CN, —NO₂, —SF₅, —SO₂NH₂, —SO₂NHR^{Z7}, —SO₂NR^{Z7}R^{Z8}, —NH—SO₂—R^{Z9}, —NR^{Z7}—SO₂—R^{Z9}, —S—R^{Z9}, —S(═O)—R^{Z9}, —SO₂—R^{Z9}, —NH₂, —NHR^{Z7}, —NR^{Z7}R^{Z8}, —OH, —O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—C(═O)—R^{Z9}, —NR^{Z7}—C(═O)—R^{Z9}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, oxo (═O), wherein 1 or 2 non-adjacent CH₂ groups of that divalent alkylene radical may be replaced independently from each other by O, S, —N(H) or N—R^{Z7}, and/or 1 or 2 non-adjacent CH groups of that divalent alkylene radical may be replaced by N;
R^{W4}, R^{W6}, R^{W6} denote Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}—Ar^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{Z}-LA^{Z}-Hetcyc^{Y}, LA^{X}, LA^{Z}-Ar^{Y}, LA^{Z}-Hetar^{Y}, LA^{Z}-Hetcyc^{Y}, CA^{X}
   or
R^{W4} and R^{W5} form together with the nitrogen atom, which they are attached to, a 3-, 4-, 5-, 6- or 7-membered heterocycle, wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7}, R^{X8}, R^{X9}, R^{Y7}, R^{Y8}, R^{Y9}, R^{Z7}, R^{Z8}, R^{Z9} denote independently from each other straight-chain or branched C₁₋₆-alkyl, which may be unsubstituted or mono-, di- or trisubstituted with independently from each other Hal, —CN, —NO₂, —SF₅, —SO₂NH₂, —SO₂NHR^{X7v}, —SO₂NR^{X7v}R^{X8v}, —NH—SO₂—R^{X9v}, —NR^{X7v}—SO₂—R^{X9v}, —S—R^{X9v}, —S(═O)—R^{X9v}, —SO₂—R^{X9v}, —NH₂, —NHR^{X7v}, —NR^{X7v}R^{X8v}, —OH, —O—R^{X9v}, —CHO, —C(═O)—R^{X9v}, —COOH, —C(═O)—O—R^{X9v}, —C(═O)—NH₂, —C(═O)—NHR^{X7v}, —C(═O)—NR^{X7v}R^{X8v}, —NH—C(═O)—R^{X9v}, —NR^{X7v}—C(═O)—R^{X9v}, —NH—(C₁₋₃-alkylene)-C(═O)—NH₂, —NH—(C₁₋₃-alkylene)-C(═O)—NHR^{X7v}, —NH—(C₁₋₃-alkylene)-C(═O)—NR^{X7v}R^{X8v}, oxo (═O), wherein 1 or 2 non-adjacent CH₂ groups of the C₁₋₆-alkyl radical may independently from each other be replaced by O, S, N(H) or N—R^{X7v}, and/or 1 or 2 non-adjacent CH groups of the C₁₋₆-alkyl radical may independently from each other be replaced by N, or a saturated monocyclic carbocycle with 3, 4, 5, 6, 7 carbon atoms, which may be unsubstituted or mono- or disubstituted with independently from each other Hal, Ar^{X}, AX-A^{Y}, Ar^{X}-Hetar^{Y}, Ar^{X}-Hetcyc^{Y}, Ar^{X}-LA^{X}-Ar^{Y}, Ar^{X}-LA^{Z}-Hetar^{Y}, Ar^{X}-LA^{Z}-Hetcyc^{Y}, Hetar^{X}, Hetar^{X}-A^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SF₅, —SO₂NH₂, —SO₂NHR^{X7v}, —SO₂NR^{X7v}R^{X8v}, —NH—SO₂—R^{X9v}, —NR^{X7v}—SO₂—R^{X9v}, —S—R^{X9v}, —S(═O)—R^{X9v}, —SO₂—R^{X9v}, —NH₂, —NHR^{X7v}, —NR^{X7v}R^{X8v}, —OH, —O—R^{X9v}, —CHO, —C(═O)—R^{X9v}, —COOH, —C(═O)—O—R^{X9v}, —C(═O)—NH₂, —C(═O)—NHR^{X7v}, —C(═O)—NR^{X7v}R^{X8v}, —NH—C(═O)—R^{X9v}, —NR^{X7v}—C(═O)—R^{X9}V, —NH—(C₁₋₃-alkylene)-C(═O)—NH₂, —NH—(C₁₋₃-alkylene)-C(═O)—NHR^{X7v}, —NH—(C₁₋₃-alkylene)-C(═O)—NR^{X7v}R^{X9v}, oxo (═O), with the proviso that if any of the substituents of that monocyclic carbocycle is Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{Z}-LA^{Z}-Hetcyc^{Y}, LA^{X}, LA^{Z}-Ar^{Y}, LA^{Z}-Hetar^{Y}, LA^{Z}-Hetcyc^{Y}, then any radical R^{X7}, R^{X8}, R^{X9}, R^{Z7}, R^{Y8}, R^{Y9}, R^{Z7}, R^{Z8}, R^{Z9} of any substituent of Ar^{X}, Ar^{Y}, Hetar^{X}, Hetar^{Y}, Hetcyc^{X}, Hetcyc^{Y}, LA^{X} and LA^{Z} may not denote a mono- or disubstituted monocyclic carbocycle, or a saturated monocyclic heterocycle with 3, 4, 5, 6, 7 ring atoms, wherein 1 or 2 ring atom(s) is/are heteroatom(s) selected from N, O and/or S, and the remaining ring atoms are carbon atoms, wherein that heterocycle may be unsubstituted or substituted with straight-chain or branched C₁₋₆-alkyl, —C(═O)—C₁₋₆-alkyl (straight-chain or branched) and/or oxo (═O), or a phenyl, —CH₂-phenyl, -naphthyl, —CH₂-naphthyl, heteroaromatic ring system or —CH₂-heteroaromatic ring system with 5, 6, 7, 8, 9, 10, 11 ring atoms, wherein 1, 2, 3, 4, 5 of said ring atoms of said heteroaromatic ring system is/are hetero atom(s) selected from N, O and/or S, and the remaining are carbon atoms, wherein said phenyl, naphthyl or heteroaromatic ring system may be unsubstituted or mono-, di- or trisubstituted with independently from each other straight-chain or branched C₁₋₆-alkyl or —O—C₁₋₆-alkyl, Hal or —C(═O)—C₁₋₆-alkyl (straight-chain or branched);
   or
each pair R^{X7} and R^{X8}; R^{Y7} and R^{Y8}; R^{Z7} and R^{Z8} form together with the nitrogen atom, which they are attached to, a 3-, 4-, 5-, 6- or 7-membered heterocycle, wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7v}, R^{X8v}, R^{X9v} denotes independently from each other straight-chain or branched C₁₋₆-alkyl, which may be unsubstituted or mono-, di- or trisubstituted with Hal, or a unsubstituted saturated monocyclic carbocycle with 3, 4, 5, 6, 7 carbon atoms;
or
R^{X7v} and R^{X8v} form together with the nitrogen atom, which they are attached to, a 3-, 4-, 5-, 6- or 7-membered heterocycle, wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
CA^{X}, CA^{Y} denote independently from each other a saturated monocyclic carbocycle with 3, 4, 5, 6, 7 carbon atoms, wherein the carbocycle may be unsubstituted or mono- or disubstituted with independently from each other R^{CA1}, R^{CA2};
R^{CA1}, R^{CA2} denote independently from each other H, Hal, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{XB}, oxo (═O), with the proviso that if R^{CA1} or R^{CA2} denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X} Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{Z}-Ar^{Y}, LA^{Z}-Hetar^{Y}, LA^{Z}-Hetcyc^{Y}, then Ar^{X}, Ar^{Y}, Hetar^{X}, Hetar^{Y}, Hetcyc^{X}, Hetcyc^{Y} may not be substituted with CA^{X} or CA^{Y};
   Hal denotes F, Cl, Br, I;

or derivatives, N-oxides, prodrugs, solvates, tautomers or stereoisomers thereof as well as the physiologically acceptable salts of each of the foregoing, including mixtures thereof in all ratios.

350. PFKFB3 inhibitor for use in neuroprotection, wherein a PFKFB3 inhibitor is of formula (XIII)

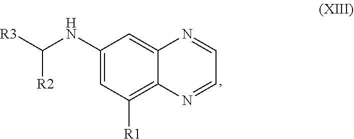

wherein R¹ denotes N-methyl-indol-6-yl (1-methyl-1H-indol-6-yl), 3-methyl-1-benzofuran-5-yl, 1-methyl-1H-pyrrolo[3,2-b]pyridin-6-yl;

R2 denotes 1H-pyrazol-4-yl or 1-methyl-1H-pyrazol-4-yl and

R3 denotes 1H-imidazol-2-yl, 1-methyl-1H-imidazol-2-yl, 1H-imidazol-5-yl, 1-methyl-1H-imidazol-5-yl, 1H-1,2,3-triazol-5-yl, 1-methyl-1H-1,2,3-triazol-5-yl, morpholin-2-yl, morpholin-3-yl, pyridin-3-yl, pyridin-4-yl, 4H-1,2,4-triazol-3-yl, 4-methyl-4H-1,2,4-triazol-3-yl; or

R2 denotes 1H-pyrazol-3-yl or 1-methyl-1H-pyrazol-3-yl and

R3 denotes 1H-1,2,3-triazol-5-yl, 1-methyl-1H-1,2,3-triazol-5-yl, 4H-1,2,4-triazol-3-yl, 4-methyl-4H-1,2,4-triazol-3-yl; or

R2 denotes 1H-pyridazin-6-on-3-yl, 6-methoxypyridazin-3-yl and

R3 denotes pyridin-3-yl, pyridin-4-yl;

or derivatives, N-oxides, prodrugs, solvates, tautomers or stereoisomers thereof as well as the physiologically acceptable salts of each of the foregoing, including mixtures thereof in all ratios.

351. PFKFB3 inhibitor for use in neuroprotection, wherein a PFKFB3 inhibitor is (2S)—N-{4-[(3-cyano-1-ethyl-1H-indol-5-yl)oxy]phenyl}-5-oxopyrrolidine-2-carboxamide

352. PFKFB3 inhibitor for use in neuroprotection, wherein a PFKFB3 inhibitor is (2RS)—N-[4-({3-cyano-1-[(3,5-dimethyl-1,2-oxazol-4-yl)methyl]-1H-indol-5-yl}oxy)phenyl]pyrrolidine-2-carboxamide.

353. PFKFB3 inhibitor for use in neuroprotection, wherein a PFKFB3 inhibitor is N-[4-({3-cyano-1-[(3,5-dimethyl-1,2-oxazol-4-yl)methyl]-1H-indol-5-yl}oxy)phenyl]pyrrolidine-2-carboxamide.

354. PFKFB3 inhibitor for use in neuroprotection, wherein a PFKFB3 inhibitor is 2-amino-N-(4-{[3-(1-methyl-1H-pyrazol-4-yl)-1H-indol-5-yl]oxy}phenyl)acetamide

355. PFKFB3 inhibitor for use in neuroprotection, wherein a PFKFB3 inhibitor is 4,6,7,8-tetrahydroxy-2-(4-hydroxyphenyl)-5H-chromen-5-one

356. PFKFB3 inhibitor for use in neuroprotection wherein a PFKFB3 inhibitor is 7,8-dihydroxy-3-(4-hydroxyphenyl)-4H-chromen-4-one

357. PFKFB3 inhibitor for use in neuroprotection wherein a PFKFB3 inhibitor is ethyl 1-(5-{2-oxa-6-azaspiro[3.3]heptan-6-yl}-6-oxo-1-phenyl-1,6-dihydropyridazin-4-yl)-1H-1,2,3-triazole-4-carboxylate

358. PFKFB3 inhibitor for use in neuroprotection, wherein a PFKFB3 inhibitor is 4-[4-(4-acetyl-5-methyl-1H-1,2,3-triazol-1-yl)-5-bromo-6-oxo-1,6-dihydropyridazin-1-yl]benzonitrile

359. PFKFB3 inhibitor for use in neuroprotection, wherein a PFKFB3 inhibitor is 5-(4-acetyl-5-methyl-1H-1,2,3-triazol-1-yl)-4-bromo-2-[4-(trifluoromethoxy)phenyl]-2,3-dihydropyridazin-3-one

360. PFKFB3 inhibitor for use in neuroprotection, wherein a PFKFB3 inhibitor is 5-(4-acetyl-5-methyl-1H-1,2,3-triazol-1-yl)-4-bromo-2-(4-chlorophenyl)-2,3-dihydropyridazin-3-one

361. PFKFB3 inhibitor for use in neuroprotection, wherein a PFKFB3 inhibitor is 5-(4-acetyl-5-methyl-1H-1,2,3-triazol-1-yl)-4-bromo-2-(pyrimidin-5-yl)-2,3-dihydropyridazin-3-one

362. PFKFB3 inhibitor for use in neuroprotection, wherein a PFKFB3 inhibitor is 5-(4-acetyl-5-methyl-1H-1,2,3-triazol-1-yl)-2-benzyl-4-bromo-2,3-dihydropyridazin-3-one

363. PFKFB3 inhibitor for use in neuroprotection, wherein a PFKFB3 inhibitor is 5-(4-acetyl-5-methyl-1H-1,2,3-triazol-1-yl)-4-bromo-2-[(4-iodophenyl)methyl]-2,3-dihydropyridazin-3-one

364. PFKFB3 inhibitor for use in neuroprotection, wherein a PFKFB3 inhibitor is 5-(4-acetyl-5-methyl-1H-1,2,3-triazol-1-yl)-4-bromo-2-(2-phenylethyl)-2,3-dihydropyridazin-3-one

365. PFKFB3 inhibitor for use in neuroprotection, wherein a PFKFB3 inhibitor is 5-(4-acetyl-5-methyl-1H-1,2,3-triazol-1-yl)-4-bromo-2-(3-phenylpropyl)-2,3-dihydropyridazin-3-one

366. PFKFB3 inhibitor for use in neuroprotection, wherein a PFKFB3 inhibitor is 2-hydroxy-4-(naphthalene-1-sulfonamido)benzoic acid 367. PFKFB3 inhibitor for use in neuroprotection, wherein a PFKFB3 inhibitor is 5-(4-acetyl-5-methyl-1H-1,2,3-triazol-1-yl)-4-bromo-2-phenyl-2,3-dihydropyridazin-3-one

368. PFKFB3 inhibitor for use in neuroprotection, wherein a PFKFB3 inhibitor is 5-(4-acetyl-5-methyl-1H-1,2,3-triazol-1-yl)-4-chloro-2-phenyl-2,3-dihydropyridazin-3-one

369. PFKFB3 inhibitor for use in neuroprotection, wherein a PFKFB3 inhibitor is 5-(4-acetyl-5-methyl-1H-1,2,3-triazol-1-yl)-4-iodo-2-phenyl-2,3-dihydropyridazin-3-one

370. PFKFB3 inhibitor for use in neuroprotection, wherein a PFKFB3 inhibitor is (2S)—N-(4-{[1-methyl-3-(1-methyl-1H-pyrazol-4-yl)-1H-indol-5-yl]oxy}phenyl)pyrrolidine-2-carboxamide

371. PFKFB3 inhibitor for use in neuroprotection, wherein a PFKFB3 inhibitor is (2S)—N-(4-{[3-cyano-1-(2-methylpropyl)-1H-indol-5-yl]amino}phenyl)pyrrolidine-2-carboxamide

372. PFKFB3 inhibitor for use in neuroprotection, wherein a PFKFB3 inhibitor is (2S)—N-(4-{[3-cyano-1-(2-methylpropyl)-1H-indol-5-yl](methyl)amino}phenyl)pyrrolidine-2-carboxamide

373. PFKFB3 inhibitor for use in neuroprotection, wherein a PFKFB3 inhibitor is (2S)—N-(4-{[3-cyano-1-(2-methylpropyl)-1H-indol-5-yl]sulfanyl}phenyl)pyrrolidine-2-carboxamide

374. PFKFB3 inhibitor for use in neuroprotection, wherein a PFKFB3 inhibitor is (2S)—N-(4-{[3-cyano-1-(2-methylpropyl)-1H-indol-5-yl]sulfonyl}phenyl)pyrrolidine-2-carboxamide

375. PFKFB3 inhibitor for use in neuroprotection, wherein a PFKFB3 inhibitor is (2S)—N-(4-{[3-cyano-1-(2-methylpropyl)-1H-indol-5-yl]methyl}phenyl)pyrrolidine-2-carboxamide

376. PFKFB3 inhibitor for use in neuroprotection, wherein a PFKFB3 inhibitor is (2S)—N-(4-{[3-cyano-1-(2-methylpropyl)-1H-indol-5-yl]oxy}phenyl)pyrrolidine-2-carboxamide

377. PFKFB3 inhibitor for use in neuroprotection, wherein a PFKFB3 inhibitor is 2-amino-N-{4-[(2-amino-3-cyano-1-ethyl-1H-indol-5-yl)oxy]phenyl}acetamide

378. PFKFB3 inhibitor for use in neuroprotection, wherein a PFKFB3 inhibitor is 2-amino-N-{4-[(2-amino-3-cyano-1-methyl-1H-indol-5-yl)oxy]phenyl}acetamide

379. PFKFB3 inhibitor for use in neuroprotection, wherein a PFKFB3 inhibitor is (2S)-2-amino-N-{4-[(2-amino-3-cyano-1H-indol-5-yl)oxy]phenyl}-3-hydroxypropanamide

380. PFKFB3 inhibitor for use in neuroprotection, wherein a PFKFB3 inhibitor is 2-amino-N-{4-[(2-amino-3-cyano-1H-indol-5-yl)oxy]phenyl}acetamide

381. PFKFB3 inhibitor for use in neuroprotection, wherein a PFKFB3 inhibitor is 2-amino-N-(4-{[2-amino-3-cyano-1-(2-methylpropyl)-1H-indol-5-yl]oxy}phenyl)acetamide

382. PFKFB3 inhibitor for use in neuroprotection, wherein a PFKFB3 inhibitor is 2-amino-N-{4-[(2-amino-1-benzyl-3-cyano-1H-indol-5-yl)oxy]phenyl}acetamide

383. PFKFB3 inhibitor for use in neuroprotection, wherein a PFKFB3 inhibitor is 2-{2-amino-5-[4-(2-aminoacetamido)phenoxy]-3-cyano-1H-indol-1-yl}-N,N-dimethylacetamide

384. PFKFB3 inhibitor for use in neuroprotection, wherein a PFKFB3 inhibitor is 2-amino-N-{4-[(3-cyano-1H-indol-5-yl)oxy]phenyl}acetamide

385. PFKFB3 inhibitor for use in neuroprotection, wherein a PFKFB3 inhibitor is 2-amino-N-{4-[(3-cyano-1-ethyl-1H-indol-5-yl)oxy]phenyl}acetamide

386. PFKFB3 inhibitor for use in neuroprotection, wherein a PFKFB3 inhibitor is N-{4-[(3-cyano-1-ethyl-1H-indol-5-yl)oxy]phenyl}-2-(methylamino)acetamide

387. PFKFB3 inhibitor for use in neuroprotection, wherein a PFKFB3 inhibitor is N-{4-[(3-cyano-1-ethyl-1H-indol-5-yl)oxy]phenyl}-2-(dimethylamino)acetamide

388. PFKFB3 inhibitor for use in neuroprotection, wherein a PFKFB3 inhibitor is (2S)—N-{4-[(3-cyano-1-ethyl-1H-indol-5-yl)oxy]phenyl}pyrrolidine-2-carboxamide

389. PFKFB3 inhibitor for use in neuroprotection, wherein a PFKFB3 inhibitor is (2R)—N-{4-[(3-cyano-1-ethyl-1H-indol-5-yl)oxy]phenyl}pyrrolidine-2-carboxamide

390. PFKFB3 inhibitor for use in neuroprotection, wherein a PFKFB3 inhibitor is (2S)—N-{4-[(3-cyano-1-ethyl-1H-indol-5-yl)oxy]phenyl}-N-methylpyrrolidine-2-carboxamide

391. PFKFB3 inhibitor for use in neuroprotection, wherein a PFKFB3 inhibitor is (2S)—N-{4-[(3-cyano-1-ethyl-1H-indol-5-yl)oxy]phenyl}azetidine-2-carboxamide

392. PFKFB3 inhibitor for use in neuroprotection, wherein a PFKFB3 inhibitor is (2S)—N-{4-[(3-cyano-1-ethyl-1H-indol-5-yl)oxy]phenyl}piperidine-2-carboxamide

393. PFKFB3 inhibitor for use in neuroprotection, wherein a PFKFB3 inhibitor is (2S)—N-{4-[(3-cyano-1-ethyl-1H-indol-5-yl)oxy]phenyl}-N-methyl-5-oxopyrrolidine-2-carboxamide

394. PFKFB3 inhibitor for use in neuroprotection, wherein a PFKFB3 inhibitor is (2S)—N-[4-({3-cyano-1-[(methylcarbamoyl)methyl]-1H-indol-5-yl}oxy)phenyl]pyrrolidine-2-carboxamide

395. PFKFB3 inhibitor for use in neuroprotection, wherein a PFKFB3 inhibitor is (2S)—N-[4-({3-cyano-1-[2-(dimethylamino)ethyl]-1H-indol-5-yl}oxy)phenyl]pyrrolidine-2-carboxamide

396. PFKFB3 inhibitor for use in neuroprotection, wherein a PFKFB3 inhibitor is (2S)—N-[4-({3-cyano-1-[(oxan-4-yl)methyl]-1H-indol-5-yl}oxy)phenyl]pyrrolidine-2-carboxamide

397. PFKFB3 inhibitor for use in neuroprotection, wherein a PFKFB3 inhibitor is (2S)—N-{4-[(3-cyano-1-phenyl-1H-indol-5-yl)oxy]phenyl}pyrrolidine-2-carboxamide

398. PFKFB3 inhibitor for use in neuroprotection, wherein a PFKFB3 inhibitor is (2S)—N-(4-{[3-cyano-1-(2-methyl-1-oxo-2,3-dihydro-1H-isoindol-5-yl)-1H-indol-5-yl]oxy}phenyl)pyrrolidine-2-carboxamide

399. PFKFB3 inhibitor for use in neuroprotection, wherein a PFKFB3 inhibitor is (2S)—N-{4-[(1-benzyl-3-cyano-1H-indol-5-yl)oxy]phenyl}pyrrolidine-2-carboxamide

400. PFKFB3 inhibitor for use in neuroprotection, wherein a PFKFB3 inhibitor is (2S)—N-[4-({3-cyano-1-[(3,5-dimethyl-1,2-oxazol-4-yl)methyl]-1H-indol-5-yl}oxy)phenyl]pyrrolidine-2-carboxamide

401. PFKFB3 inhibitor for use in neuroprotection, wherein a PFKFB3 inhibitor is (2S)—N-(4-{[3-cyano-1-(2-methylpropyl)-1H-indazol-5-yl]oxy}phenyl)pyrrolidine-2-carboxamide

402. PFKFB3 inhibitor for use in neuroprotection, wherein a PFKFB3 inhibitor is (2S)—N-[4-({3-cyano-1-[(3,5-dimethyl-1,2-oxazol-4-yl)methyl]-1H-indazol-5-yl}oxy)phenyl]pyrrolidine-2-carboxamide

403. PFKFB3 inhibitor for use in neuroprotection, wherein a PFKFB3 inhibitor is 2-amino-N-(4-{[3-(1-methyl-1H-pyrazol-4-yl)-1H-indazol-5-yl]oxy}phenyl)acetamide

404. PFKFB3 inhibitor for use in neuroprotection, wherein a PFKFB3 inhibitor is (2S)—N-(4-{[3-(1-methyl-1H-pyrazol-4-yl)-1H-indol-5-yl]oxy}phenyl)pyrrolidine-2-carboxamide

405. PFKFB3 inhibitor for use in neuroprotection, wherein a PFKFB3 inhibitor is N-{1-[3-(2-aminopyrimidin-5-yl)-4-methylphenyl]ethyl}-2-methyl-6-(3-methyl-1-benzofuran-5-yl)pyrimidin-4-amine

406. PFKFB3 inhibitor for use in neuroprotection, wherein a PFKFB3 inhibitor is 6-(1,3-benzothiazol-5-yl)-2-methyl-N-{1-[3-(1-methyl-1H-pyrazol-4-yl)phenyl]ethyl}pyrimidin-4-amine

407. PFKFB3 inhibitor for use in neuroprotection, wherein a PFKFB3 inhibitor is [1-(5-{3-[(1S)-1-{[2-methyl-6-(3-methyl-1-benzofuran-5-yl)pyrimidin-4-yl]amino}ethyl]phenyl}pyrimidin-2-yl)piperidin-4-yl]methanol

408. PFKFB3 inhibitor for use in neuroprotection, wherein a PFKFB3 inhibitor is 2-methyl-N-{1-[3-(1-methyl-1H-pyrazol-4-yl)phenyl]ethyl}-6-phenylpyrimidin-4-amine

409. PFKFB3 inhibitor for use in neuroprotection, wherein a PFKFB3 inhibitor is 6-(1H-indol-6-yl)-2-methyl-N-[(1R)-1,2,3,4-tetrahydronaphthalen-1-yl]pyrimidin-4-amine

410. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]acetonitrile

411. PFKFB3 inhibitor for use in neuroprotection, wherein a PFKFB3 inhibitor is 6-(1,3-benzothiazol-6-yl)-2-methyl-N-(1-{3-[(1,2-oxazol-3-yl)methoxy]phenyl}ethyl)pyrimidin-4-amine

412. PFKFB3 inhibitor for use in neuroprotection, wherein a PFKFB3 inhibitor is 6-(2,3-dihydro-1-benzofuran-5-yl)-N-[(1S)-1-(3-methoxyphenyl)ethyl]-2-methylpyrimidin-4-amine

413. PFKFB3 inhibitor for use in neuroprotection, wherein a PFKFB3 inhibitor is 2-methyl-6-(3-methyl-1-benzofuran-5-yl)-N-[(1S)-1-[5-(1-methyl-1H-pyrazol-4-yl)pyridin-3-yl]ethyl]pyrimidin-4-amine

414. PFKFB3 inhibitor for use in neuroprotection, wherein a PFKFB3 inhibitor is 2-methyl-N-(1-{3-[(5-methyl-1,2,4-oxadiazol-3-yl)methoxy]phenyl}ethyl)-6-(3-methyl-1-benzofuran-5-yl)pyrimidin-4-amine

415. PFKFB3 inhibitor for use in neuroprotection, wherein a PFKFB3 inhibitor is 6-(2-fluoro-3-methoxyphenyl)-2-methyl-N-{1-[3-(1-methyl-1H-pyrazol-4-yl)phenyl]ethyl}pyrimidin-4-amine

416. PFKFB3 inhibitor for use in neuroprotection, wherein a PFKFB3 inhibitor is N-{1-[5-(2-aminopyrimidin-5-yl)pyridin-3-yl]ethyl}-2-methyl-6-(3-methyl-1-benzofuran-5-yl)pyrimidin-4-amine

417. PFKFB3 inhibitor for use in neuroprotection, wherein a PFKFB3 inhibitor is N-[(1S)-1-[3-(5-chloropyridin-3-yl)phenyl]ethyl]-2-methyl-6-(3-methyl-1-benzofuran-5-yl)pyrimidin-4-amine

418. PFKFB3 inhibitor for use in neuroprotection, wherein a PFKFB3 inhibitor is N-[(1S)-1-[3-(4-chloropyridin-3-yl)phenyl]ethyl]-2-methyl-6-(3-methyl-1-benzofuran-5-yl)pyrimidin-4-amine

419. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-ethyl-N-methylacetamide

420. PFKFB3 inhibitor for use in neuroprotection, wherein a PFKFB3 inhibitor is 6-{4-chloro-3-[(prop-2-yn-1-yl)amino]phenyl}-2-methyl-N-[(1R)-1,2,3,4-tetrahydronaphthalen-1-yl]pyrimidin-4-amine

421. PFKFB3 inhibitor for use in neuroprotection, wherein a PFKFB3 inhibitor is N-[(3-methoxyphenyl)methyl]-2-methyl-6-(3-methyl-1-benzofuran-5-yl)pyrimidin-4-amine

422. PFKFB3 inhibitor for use in neuroprotection, wherein a PFKFB3 inhibitor is 2-methyl-6-(2-phenylethenyl)-N-[(1R)-1,2,3,4-tetrahydronaphthalen-1-yl]pyrimidin-4-amine

423. PFKFB3 inhibitor for use in neuroprotection, wherein a PFKFB3 inhibitor is 4-(2-methyl-6-{[(1R)-1,2,3,4-tetrahydronaphthalen-1-yl]amino}pyrimidin-4-yl)benzamide

424. PFKFB3 inhibitor for use in neuroprotection, wherein a PFKFB3 inhibitor is 3-({[6-(1,3-benzothiazol-6-yl)-2-methylpyrimidin-4-yl]amino}methyl)-4-fluorophenol

425. PFKFB3 inhibitor for use in neuroprotection, wherein a PFKFB3 inhibitor is 2-(5-{3-[(1S)-1-{[2-methyl-6-(3-methyl-1-benzofuran-5-yl)pyrimidin-4-yl]amino}ethyl]phenyl}pyridin-3-yl)propan-2-ol

426. PFKFB3 inhibitor for use in neuroprotection, wherein a PFKFB3 inhibitor is 6-(3-ethyl-1-benzofuran-5-yl)-N-[(1S)-1-(3-methoxyphenyl)ethyl]-2-methylpyrimidin-4-amine

427. PFKFB3 inhibitor for use in neuroprotection, wherein a PFKFB3 inhibitor is 6-(1,3-benzothiazol-6-yl)-N-[(2-chloro-6-fluorophenyl)methyl]-2-methylpyrimidin-4-amine

428. PFKFB3 inhibitor for use in neuroprotection, wherein a PFKFB3 inhibitor is 2-methyl-6-(3-methyl-1-benzofuran-5-yl)-N-[(1S)-1-{3-[2-(morpholin-4-yl)pyrimidin-5-yl]phenyl}ethyl]pyrimidin-4-amine

429. PFKFB3 inhibitor for use in neuroprotection, wherein a PFKFB3 inhibitor is N1-[6-(1,3-benzothiazol-6-yl)-2-methylpyrimidin-4-yl]-1,2,3,4-tetrahydronaphthalene-1,6-diamine

430. PFKFB3 inhibitor for use in neuroprotection, wherein a PFKFB3 inhibitor is 2-methyl-6-(3-methyl-1-benzofuran-5-yl)-N-[(1S)-1-[3-(1H-1,2,3,4-tetrazol-1-yl)phenyl]ethyl]pyrimidin-4-amine

431. PFKFB3 inhibitor for use in neuroprotection, wherein a PFKFB3 inhibitor is 6-(1,3-benzothiazol-6-yl)-N-[(1S)-1-[3-(5-chloropyridin-3-yl)phenyl]ethyl]-2-methylpyrimidin-4-amine

432. PFKFB3 inhibitor for use in neuroprotection, wherein a PFKFB3 inhibitor is 2-methyl-6-(3-methyl-1-benzofuran-5-yl)-N-[(1S)-1-[3-(1H-pyrazol-3-yl)phenyl]ethyl]pyrimidin-4-amine

433. PFKFB3 inhibitor for use in neuroprotection, wherein a PFKFB3 inhibitor is N-[(1S)-1-{3-[6-(ethylamino)pyridin-3-yl]phenyl}ethyl]-2-methyl-6-(3-methyl-1-benzofuran-5-yl)pyrimidin-4-amine

434. PFKFB3 inhibitor for use in neuroprotection, wherein a PFKFB3 inhibitor is N-(cyanomethyl)-3-(1-{[2-methyl-6-(1-methyl-1H-indol-6-yl)pyrimidin-4-yl]amino}ethyl)benzene-1-sulfonamide

435. PFKFB3 inhibitor for use in neuroprotection, wherein a PFKFB3 inhibitor is N-[3-(1-{[6-(1,3-benzothiazol-6-yl)-2-methylpyrimidin-4-yl]amino}ethyl)phenyl]-1H-pyrazole-5-carboxamide

436. PFKFB3 inhibitor for use in neuroprotection, wherein a PFKFB3 inhibitor is 6-(1,3-benzothiazol-6-yl)-N-[(2-bromo-5-methoxyphenyl)methyl]-2-methylpyrimidin-4-amine

437. PFKFB3 inhibitor for use in neuroprotection, wherein a PFKFB3 inhibitor is 3-[(5-{3-[(1S)-1-{[6-(7-fluoro-3-methyl-1-benzofuran-5-yl)-2-methylpyrimidin-4-yl]amino}ethyl]phenyl}pyrimidin-2-yl)amino]propane-1,2-diol

438. PFKFB3 inhibitor for use in neuroprotection, wherein a PFKFB3 inhibitor is 6-(1,3-benzothiazol-6-yl)-2-methyl-N-(1-{3-[(piperidin-3-yl)methoxy]phenyl}ethyl)pyrimidin-4-amine

439. PFKFB3 inhibitor for use in neuroprotection, wherein a PFKFB3 inhibitor is methyl 2-({5-[3-(1-{[2-methyl-6-(3-methyl-1-benzofuran-5-yl)pyrimidin-4-yl]amino}ethyl)phenyl]pyrimidin-2-yl}amino)acetate

440. PFKFB3 inhibitor for use in neuroprotection, wherein a PFKFB3 inhibitor is 2-methyl-6-(3-methyl-1-benzofuran-5-yl)-N-[(1S)-1-(3-{2-[(propan-2-yl)amino]pyrimidin-5-yl}phenyl)ethyl]pyrimidin-4-amine

441. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-ethylacetamide

442. PFKFB3 inhibitor for use in neuroprotection, wherein a PFKFB3 inhibitor is 6-(3-amino-4-chlorophenyl)-2-methyl-N-{1-[3-(1-methyl-1H-pyrazol-4-yl)phenyl]ethyl}pyrimidin-4-amine

443. PFKFB3 inhibitor for use in neuroprotection, wherein a PFKFB3 inhibitor is N-[(1S)-1-[3-(5-aminopyridin-3-yl)phenyl]ethyl]-2-methyl-6-(3-methyl-1-benzothiophen-5-yl)pyrimidin-4-amine

444. PFKFB3 inhibitor for use in neuroprotection, wherein a PFKFB3 inhibitor is 2-methyl-6-{1-methyl-1H-pyrrolo[3,2-b]pyridin-6-yl}-N-[(1R)-1,2,3,4-tetrahydronaphthalen-1-yl]pyrimidin-4-amine

445. PFKFB3 inhibitor for use in neuroprotection, wherein a PFKFB3 inhibitor is 6-(3,4-dichlorophenyl)-2-methyl-N-{1-[3-(1-methyl-1H-pyrazol-4-yl)phenyl]ethyl}pyrimidin-4-amine

446. PFKFB3 inhibitor for use in neuroprotection, wherein a PFKFB3 inhibitor is N-[(1S)-1-(3-bromophenyl)ethyl]-6-(3-ethyl-1-benzofuran-5-yl)-2-methylpyrimidin-4-amine

447. PFKFB3 inhibitor for use in neuroprotection, wherein a PFKFB3 inhibitor is 6-(1,3-benzothiazol-6-yl)-N-[(1S)-1-{3-[2-(dimethylamino)pyrimidin-5-yl]phenyl}ethyl]-2-methylpyrimidin-4-amine

448. PFKFB3 inhibitor for use in neuroprotection, wherein a PFKFB3 inhibitor is 6-(1,3-benzothiazol-6-yl)-2-methyl-N-[(1S)-1-[3-(1H-pyrazol-4-yl)phenyl]ethyl]pyrimidin-4-amine

449. PFKFB3 inhibitor for use in neuroprotection, wherein a PFKFB3 inhibitor is 2-methyl-6-[4-methyl-3-(methylamino)phenyl]-N-[(1R)-1,2,3,4-tetrahydronaphthalen-1-yl]pyrimidin-4-amine

450. PFKFB3 inhibitor for use in neuroprotection, wherein a PFKFB3 inhibitor is N-(1-{3-[(1,3-dimethyl-1H-pyrazol-5-yl)methoxy]phenyl}ethyl)-2-methyl-6-(3-methyl-1-benzofuran-5-yl)pyrimidin-4-amine

451. PFKFB3 inhibitor for use in neuroprotection, wherein a PFKFB3 inhibitor is 6-(1,3-benzothiazol-6-yl)-2-methyl-N-{1-[3-(1H-pyrrol-2-yl)phenyl]ethyl}pyrimidin-4-amine

452. PFKFB3 inhibitor for use in neuroprotection, wherein a PFKFB3 inhibitor is 3-(1-{[6-(2-chloro-3-methoxyphenyl)-2-methylpyrimidin-4-yl]amino}ethyl)benzene-1-sulfonamide

453. PFKFB3 inhibitor for use in neuroprotection, wherein a PFKFB3 inhibitor is 2-methyl-6-(3-methyl-1-benzofuran-5-yl)-N-[1-(3-nitrophenyl)ethyl]pyrimidin-4-amine

454. PFKFB3 inhibitor for use in neuroprotection, wherein a PFKFB3 inhibitor is 6-(1,3-benzothiazol-6-yl)-2-methyl-N-{1-[3-(prop-2-yn-1-yloxy)phenyl]ethyl}pyrimidin-4-amine

455. PFKFB3 inhibitor for use in neuroprotection, wherein a PFKFB3 inhibitor is 6-(1,3-benzothiazol-6-yl)-N-[1-(2-chloropyridin-4-yl)ethyl]-2-methylpyrimidin-4-amine

456. PFKFB3 inhibitor for use in neuroprotection, wherein a PFKFB3 inhibitor is 3-(1-{[6-(1,3-benzothiazol-6-yl)-2-methylpyrimidin-4-yl]amino}ethyl)-N-(cyanomethyl)benzene-1-sulfonamide

457. PFKFB3 inhibitor for use in neuroprotection, wherein a PFKFB3 inhibitor is 2-methyl-6-(3-methyl-1-benzofuran-5-yl)-N-[(1S)-1-[2-(1-methyl-1H-pyrazol-4-yl)pyridin-4-yl]ethyl]pyrimidin-4-amine

458. PFKFB3 inhibitor for use in neuroprotection, wherein a PFKFB3 inhibitor is 6-(1-benzofuran-5-yl)-2-methyl-N-{1-[3-(1-methyl-1H-pyrazol-4-yl)phenyl]ethyl}pyrimidin-4-amine

459. PFKFB3 inhibitor for use in neuroprotection, wherein a PFKFB3 inhibitor is 6-(4-fluorophenyl)-2-methyl-N-[(1R)-1,2,3,4-tetrahydronaphthalen-1-yl]pyrimidin-4-amine

460. PFKFB3 inhibitor for use in neuroprotection, wherein a PFKFB3 inhibitor is 3-{5-[2-(methylsulfanyl)pyrimidin-5-yl]thiophene-2-sulfonamido}benzoic acid

461. PFKFB3 inhibitor for use in neuroprotection, wherein a PFKFB3 inhibitor is 2-{[3-(1-{[6-(1,3-benzothiazol-6-yl)-2-methylpyrimidin-4-yl]amino}ethyl)phenoxy]methyl}-1,3-oxazole-4-carboxylic acid

462. PFKFB3 inhibitor for use in neuroprotection, wherein a PFKFB3 inhibitor is 6-(7-fluoro-3-methyl-1-benzofuran-5-yl)-N-[(1S)-1-[3-(5-fluoropyridin-3-yl)phenyl]ethyl]-2-methylpyrimidin-4-amine

463. PFKFB3 inhibitor for use in neuroprotection, wherein a PFKFB3 inhibitor is 2-[3-(1-{[6-(3-methoxyphenyl)-2-methylpyrimidin-4-yl]amino}ethyl)phenoxy]-N-methylacetamide

464. PFKFB3 inhibitor for use in neuroprotection, wherein a PFKFB3 inhibitor is 6-(1,3-benzothiazol-6-yl)-2-methyl-N-(1-{3-[(5-methyl-1,2,4-oxadiazol-3-yl)methoxy]phenyl}ethyl)pyrimidin-4-amine

465. PFKFB3 inhibitor for use in neuroprotection, wherein a PFKFB3 inhibitor is 6-(1,3-benzothiazol-6-yl)-2-methyl-N-{1-[3-(2,2,2-trifluoroethoxy)phenyl]ethyl}pyrimidin-4-amine

466. PFKFB3 inhibitor for use in neuroprotection, wherein a PFKFB3 inhibitor is N-(cyanomethyl)-3-(1-{[6-(3-methoxyphenyl)-2-methylpyrimidin-4-yl]amino}ethyl)benzene-1-sulfonamide

467. PFKFB3 inhibitor for use in neuroprotection, wherein a PFKFB3 inhibitor is 6-(3-chlorophenyl)-2-methyl-N-[(1R)-1,2,3,4-tetrahydronaphthalen-1-yl]pyrimidin-4-amine

468. PFKFB3 inhibitor for use in neuroprotection, wherein a PFKFB3 inhibitor is 2-methyl-6-(3-methyl-1-benzofuran-5-yl)-N-[(1R)-1-[5-(1-methyl-1H-pyrazol-4-yl)pyridin-3-yl]ethyl]pyrimidin-4-amine

469. PFKFB3 inhibitor for use in neuroprotection, wherein a PFKFB3 inhibitor is 1-(5-{3-[(1S)-1-{[2-methyl-6-(3-methyl-1-benzofuran-5-yl)pyrimidin-4-yl]amino}ethyl]phenyl}pyrimidin-2-yl)piperidin-4-ol

470. PFKFB3 inhibitor for use in neuroprotection, wherein a PFKFB3 inhibitor is 2-[2-methyl-5-(2-methyl-6-{[(1R)-1,2,3,4-tetrahydronaphthalen-1-yl]amino}pyrimidin-4-yl)phenoxy]acetonitrile

471. PFKFB3 inhibitor for use in neuroprotection, wherein a PFKFB3 inhibitor is 2-{[2-chloro-5-(2-methyl-6-{[(1R)-1,2,3,4-tetrahydronaphthalen-1-yl]amino}pyrimidin-4-yl)phenyl]methoxy}propanoic acid

472. PFKFB3 inhibitor for use in neuroprotection, wherein a PFKFB3 inhibitor is N-[(1S)-1-[3-(2-aminopyrimidin-5-yl)phenyl]ethyl]-6-(3,4-difluorophenyl)-2-methylpyrimidin-4-amine

473. PFKFB3 inhibitor for use in neuroprotection, wherein a PFKFB3 inhibitor is 6-(1,3-benzothiazol-6-yl)-N-(1-{3-[2-(dimethylamino)ethoxy]phenyl}ethyl)-2-methylpyrimidin-4-amine

474. PFKFB3 inhibitor for use in neuroprotection wherein a PFKFB3 inhibitor is 3-(1-{[2-methyl-6-(1-methyl-1H-indol-6-yl)pyrimidin-4-yl]amino}ethyl)phenol

475. PFKFB3 inhibitor for use in neuroprotection wherein a PFKFB3 inhibitor is 1-(3-{[3-(1-{[6-(1,3-benzothiazol-6-yl)-2-methylpyrimidin-4-yl]amino}ethyl)phenoxy]methyl}piperidin-1-yl)ethan-1-one

476. PFKFB3 inhibitor for use in neuroprotection, wherein a PFKFB3 inhibitor is 2-fluoro-5-(1-{[2-methyl-6-(3-methyl-1-benzofuran-5-yl)pyrimidin-4-yl]amino}ethyl)phenol

477. PFKFB3 inhibitor for use in neuroprotection, wherein a PFKFB3 inhibitor is ethyl 4-[3-(1-{[2-methyl-6-(1-methyl-1H-indol-6-yl)pyrimidin-4-yl]amino}ethyl)phenoxy]butanoate

478. PFKFB3 inhibitor for use in neuroprotection, wherein a PFKFB3 inhibitor is [4-(2-methyl-6-{[(1R)-1,2,3,4-tetrahydronaphthalen-1-yl]amino}pyrimidin-4-yl)phenyl]methanol

479. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-cyclohexylacetamide

480. PFKFB3 inhibitor for use in neuroprotection, wherein a PFKFB3 inhibitor is 6-(1,3-benzothiazol-6-yl)-2-methyl-N-[(1S)-1-[3-(5-methylpyridin-3-yl)phenyl]ethyl]pyrimidin-4-amine

481. PFKFB3 inhibitor for use in neuroprotection, wherein a PFKFB3 inhibitor is 2-methyl-6-(1-methyl-1H-indol-6-yl)-N-[(1S)-1-(4-methylphenyl)ethyl]pyrimidin-4-amine

482. PFKFB3 inhibitor for use in neuroprotection, wherein a PFKFB3 inhibitor is N-[(1S)-1-[3-(1-ethyl-1H-pyrazol-4-yl)phenyl]ethyl]-2-methyl-6-(3-methyl-1-benzofuran-5-yl)pyrimidin-4-amine

483. PFKFB3 inhibitor for use in neuroprotection, wherein a PFKFB3 inhibitor is N-[(1S)-1-[3-(6-fluoropyridin-3-yl)phenyl]ethyl]-2-methyl-6-(3-methyl-1-benzofuran-5-yl)pyrimidin-4-amine

484. PFKFB3 inhibitor for use in neuroprotection, wherein a PFKFB3 inhibitor is 3-[(5-{3-[(1S)-1-{[6-(4-chloro-3-fluorophenyl)-2-methylpyrimidin-4-yl]amino}ethyl]phenyl}pyrimidin-2-yl)amino]propane-1,2-diol

485. PFKFB3 inhibitor for use in neuroprotection, wherein a PFKFB3 inhibitor is 6-(1H-indol-5-yl)-N-[(1S)-1-(3-methoxyphenyl)ethyl]-2-methylpyrimidin-4-amine

486. PFKFB3 inhibitor for use in neuroprotection, wherein a PFKFB3 inhibitor is 2-[3-(1-{[6-(2H-1,3-benzodioxol-5-yl)-2-methylpyrimidin-4-yl]amino}ethyl)phenoxy]-N-methylacetamide

487. PFKFB3 inhibitor for use in neuroprotection, wherein a PFKFB3 inhibitor is 2-methyl-6-(1-methyl-1H-1,3-benzodiazol-6-yl)-N-[(1R)-1,2,3,4-tetrahydronaphthalen-1-yl]pyrimidin-4-amine

488. PFKFB3 inhibitor for use in neuroprotection, wherein a PFKFB3 inhibitor is 6-(3-amino-4-methylphenyl)-2-methyl-N-[(1R)-1,2,3,4-tetrahydronaphthalen-1-yl]pyrimidin-4-amine

489. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-propylacetamide

490. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-methylacetamide

491. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]acetamide

492. PFKFB3 inhibitor for use in neuroprotection, wherein a PFKFB3 inhibitor is 2-methyl-6-(quinolin-6-yl)-N-[(1R)-1,2,3,4-tetrahydronaphthalen-1-yl]pyrimidin-4-amine

493. PFKFB3 inhibitor for use in neuroprotection, wherein a PFKFB3 inhibitor is 5-{3-[(1S)-1-{[2-methyl-6-(3-methyl-1-benzofuran-5-yl)pyrimidin-4-yl]amino}ethyl]phenyl}pyridine-3-carbonitrile

494. PFKFB3 inhibitor for use in neuroprotection, wherein a PFKFB3 inhibitor is N,N-diethyl-2-[3-(1-{[2-methyl-6-(1-methyl-1H-indol-6-yl)pyrimidin-4-yl]amino}ethyl)phenoxy]acetamide

495. PFKFB3 inhibitor for use in neuroprotection, wherein a PFKFB3 inhibitor is 2-methoxy-4-(2-methyl-6-{[(1R)-1,2,3,4-tetrahydronaphthalen-1-yl]amino}pyrimidin-4-yl)benzamide

496. PFKFB3 inhibitor for use in neuroprotection, wherein a PFKFB3 inhibitor is 2-methyl-6-(2-methylphenyl)-N-[(1R)-1,2,3,4-tetrahydronaphthalen-1-yl]pyrimidin-4-amine

497. PFKFB3 inhibitor for use in neuroprotection, wherein a PFKFB3 inhibitor is N-[(1S)-1-[3-(2-aminopyrimidin-5-yl)phenyl]ethyl]-6-(7-fluoro-3-methyl-1-benzofuran-5-yl)-2-methylpyrimidin-4-amine

498. PFKFB3 inhibitor for use in neuroprotection, wherein a PFKFB3 inhibitor is 2-methyl-6-(1-methyl-1H-indol-2-yl)-N-[(1R)-1,2,3,4-tetrahydronaphthalen-1-yl]pyrimidin-4-amine

499. PFKFB3 inhibitor for use in neuroprotection, wherein a PFKFB3 inhibitor is 6-(7-fluoro-3-methyl-1-benzofuran-5-yl)-2-methyl-N-{1-[3-(1-methyl-1H-pyrazol-4-yl)phenyl]ethyl}pyrimidin-4-amine

500. PFKFB3 inhibitor for use in neuroprotection, wherein a PFKFB3 inhibitor is N-[(1S)-1-(3-bromophenyl)ethyl]-2-methyl-6-(3-methyl-1-benzothiophen-5-yl)pyrimidin-4-amine

501. PFKFB3 inhibitor for use in neuroprotection, wherein a PFKFB3 inhibitor is N-[(1S)-1-(3-methoxyphenyl)ethyl]-2-methyl-6-(1-methyl-1H-indol-6-yl)pyrimidin-4-amine

502. PFKFB3 inhibitor for use in neuroprotection, wherein a PFKFB3 inhibitor is 6-(1,3-benzothiazol-6-yl)-2-methyl-N-[(1S)-1-[3-(1-methyl-1H-pyrazol-4-yl)phenyl]ethyl]pyrimidin-4-amine

503. PFKFB3 inhibitor for use in neuroprotection, wherein a PFKFB3 inhibitor is 6-(4-chloro-3-fluorophenyl)-N-{1-[5-(1-ethyl-1H-pyrazol-4-yl)pyridin-3-yl]ethyl}-2-methylpyrimidin-4-amine

504. PFKFB3 inhibitor for use in neuroprotection, wherein a PFKFB3 inhibitor is 2-methyl-6-(1-methyl-1H-indol-6-yl)-N-[(1S)-1-phenylethyl]pyrimidin-4-amine

505. PFKFB3 inhibitor for use in neuroprotection, wherein a PFKFB3 inhibitor is 6-(1,3-benzothiazol-6-yl)-N-[(2-fluorophenyl)methyl]-2-methylpyrimidin-4-amine

506. PFKFB3 inhibitor for use in neuroprotection, wherein a PFKFB3 inhibitor is 6-(1,3-benzothiazol-6-yl)-N-(2,3-dihydro-1H-inden-2-yl)-2-methylpyrimidin-4-amine

507. PFKFB3 inhibitor for use in neuroprotection, wherein a PFKFB3 inhibitor is 6-(2,3-dihydro-1-benzofuran-5-yl)-2-methyl-N-[(1R)-1,2,3,4-tetrahydronaphthalen-1-yl]pyrimidin-4-amine

508. PFKFB3 inhibitor for use in neuroprotection, wherein a PFKFB3 inhibitor is 6-(1,3-benzothiazol-6-yl)-N-[(2-chlorophenyl)methyl]-2-methylpyrimidin-4-amine

509. PFKFB3 inhibitor for use in neuroprotection, wherein a PFKFB3 inhibitor is 2-methyl-6-(3-methyl-1-benzofuran-5-yl)-N-[(1S)-1-{3-[2-(methylamino)pyrimidin-5-yl]phenyl}ethyl]pyrimidin-4-amine

510. PFKFB3 inhibitor for use in neuroprotection, wherein a PFKFB3 inhibitor is 2-methyl-6-(3-methyl-1-benzothiophen-5-yl)-N-[(1S)-1-[3-(1-methyl-1H-pyrazol-4-yl)phenyl]ethyl]pyrimidin-4-amine

511. PFKFB3 inhibitor for use in neuroprotection, wherein a PFKFB3 inhibitor is 6-(4-chloro-3-methylphenyl)-N-[(4R)-3,4-dihydro-2H-1-benzopyran-4-yl]-2-methylpyrimidin-4-amine

512. PFKFB3 inhibitor for use in neuroprotection, wherein a PFKFB3 inhibitor is 6-(1,3-benzothiazol-6-yl)-N-{1-[3-(benzyloxy)phenyl]ethyl}-2-methylpyrimidin-4-amine

513. PFKFB3 inhibitor for use in neuroprotection, wherein a PFKFB3 inhibitor is 6-(2-chloro-3-methoxyphenyl)-N-(1-{3-[(1,3-dimethyl-1H-pyrazol-5-yl)methoxy]phenyl}ethyl)-2-methylpyrimidin-4-amine

514. PFKFB3 inhibitor for use in neuroprotection, wherein a PFKFB3 inhibitor is 2-methyl-6-(1-methyl-1H-indol-6-yl)-N-[(1R)-1,2,3,4-tetrahydronaphthalen-1-yl]pyrimidin-4-amine

515. PFKFB3 inhibitor for use in neuroprotection, wherein a PFKFB3 inhibitor is 2-methyl-6-(3-methyl-1-benzofuran-5-yl)-N-(1-{3-[(1-methyl-1H-pyrazol-3-yl)methoxy]phenyl}ethyl)pyrimidin-4-amine

516. PFKFB3 inhibitor for use in neuroprotection, wherein a PFKFB3 inhibitor is N-[(1S)-1-[5-(1-ethyl-1H-pyrazol-4-yl)pyridin-3-yl]ethyl]-2-methyl-6-(3-methyl-1-benzofuran-5-yl)pyrimidin-4-amine

517. PFKFB3 inhibitor for use in neuroprotection, wherein a PFKFB3 inhibitor is N-methyl-2-[3-(1-{[2-methyl-6-(1-methyl-1H-indol-6-yl)pyrimidin-4-yl]amino}ethyl)phenoxy]acetamide

518. PFKFB3 inhibitor for use in neuroprotection, wherein a PFKFB3 inhibitor is N-[1-(3-bromo-4-fluorophenyl)ethyl]-2-methyl-6-(3-methyl-1-benzofuran-5-yl)pyrimidin-4-amine

519. PFKFB3 inhibitor for use in neuroprotection, wherein a PFKFB3 inhibitor is N-[(1S)-1-[3-(5-aminopyridin-3-yl)phenyl]ethyl]-2-methyl-6-(3-methyl-1-benzofuran-5-yl)pyrimidin-4-amine

520. PFKFB3 inhibitor for use in neuroprotection, wherein a PFKFB3 inhibitor is N-[(1S)-1-[3-(5-aminopyridin-3-yl)phenyl]ethyl]-6-(7-fluoro-3-methyl-1-benzofuran-5-yl)-2-methylpyrimidin-4-amine

521. PFKFB3 inhibitor for use in neuroprotection, wherein a PFKFB3 inhibitor is 5-[3-(1-{[6-(1,3-benzothiazol-6-yl)-2-methylpyrimidin-4-yl]amino}ethyl)phenyl]pyrimidine-2-carbonitrile

522. PFKFB3 inhibitor for use in neuroprotection, wherein a PFKFB3 inhibitor is 6-(1,3-benzothiazol-6-yl)-2-methyl-N-(1-{3-[(pyridin-3-yl)methoxy]phenyl}ethyl)pyrimidin-4-amine

523. PFKFB3 inhibitor for use in neuroprotection, wherein a PFKFB3 inhibitor is 6-(1,3-benzothiazol-6-yl)-N-(2-ethylhexyl)-2-methylpyrimidin-4-amine

524. PFKFB3 inhibitor for use in neuroprotection, wherein a PFKFB3 inhibitor is N-[(1S)-1-[3-(2-aminopyrimidin-5-yl)phenyl]ethyl]-6-(4-chlorophenyl)-2-methylpyrimidin-4-amine

525. PFKFB3 inhibitor for use in neuroprotection, wherein a PFKFB3 inhibitor is N-(1-{3-[(1,3-dimethyl-1H-pyrazol-5-yl)methoxy]phenyl}ethyl)-6-(3-methoxy-4-methylphenyl)-2-methylpyrimidin-4-amine

526. PFKFB3 inhibitor for use in neuroprotection, wherein a PFKFB3 inhibitor is 6-(4-chlorophenyl)-2-methyl-N-{1-[3-(1-methyl-1H-pyrazol-4-yl)phenyl]ethyl}pyrimidin-4-amine

527. PFKFB3 inhibitor for use in neuroprotection, wherein a PFKFB3 inhibitor is 1-({[3-(1-{[6-(1,3-benzothiazol-6-yl)-2-methylpyrimidin-4-yl]amino}ethyl)phenyl]carbamoyl}amino)formamide

528. PFKFB3 inhibitor for use in neuroprotection, wherein a PFKFB3 inhibitor is 6-(1,3-benzothiazol-6-yl)-2-methyl-N-{1-[3-(2-methyl-1,3-thiazol-4-yl)phenyl]ethyl}pyrimidin-4-amine

529. PFKFB3 inhibitor for use in neuroprotection, wherein a PFKFB3 inhibitor is N-[(1S)-1-(3-{2-[4-(2-methoxyethyl)piperazin-1-yl]pyrimidin-5-yl}phenyl)ethyl]-2-methyl-6-(3-methyl-1-benzofuran-5-yl)pyrimidin-4-amine

530. PFKFB3 inhibitor for use in neuroprotection, wherein a PFKFB3 inhibitor is 2-[3-(1-{[2-methyl-6-(1-methyl-1H-indol-6-yl)pyrimidin-4-yl]amino}ethyl)phenoxy]-1-[4-(pyridin-2-yl)piperazin-1-yl]ethan-1-one

531. PFKFB3 inhibitor for use in neuroprotection, wherein a PFKFB3 inhibitor is 3-(1-{[6-(1,3-benzothiazol-6-yl)-2-methylpyrimidin-4-yl]amino}ethyl)-N-(prop-2-yn-1-yl)benzene-1-sulfonamide

532. PFKFB3 inhibitor for use in neuroprotection, wherein a PFKFB3 inhibitor is 6-(1,3-benzothiazol-6-yl)-2-methyl-N-[2-(pyridin-3-yl)ethyl]pyrimidin-4-amine

533. PFKFB3 inhibitor for use in neuroprotection, wherein a PFKFB3 inhibitor is N-{1-[5-(1-ethyl-1H-pyrazol-4-yl)pyridin-3-yl]ethyl}-6-(7-fluoro-3-methyl-1-benzofuran-5-yl)-2-methylpyrimidin-4-amine

534. PFKFB3 inhibitor for use in neuroprotection, wherein a PFKFB3 inhibitor is 3-[(1S)-1-{[2-methyl-6-(3-methyl-1-benzofuran-5-yl)pyrimidin-4-yl]amino}ethyl]phenol

535. PFKFB3 inhibitor for use in neuroprotection, wherein a PFKFB3 inhibitor is N-[(1S)-1-(3-methoxyphenyl)ethyl]-2-methyl-6-(1-methyl-1H-indol-2-yl)pyrimidin-4-amine

536. PFKFB3 inhibitor for use in neuroprotection, wherein a PFKFB3 inhibitor is 5-[3-(1-{[6-(1,3-benzothiazol-6-yl)-2-methylpyrimidin-4-yl]amino}ethyl)phenyl]pyridine-3-carbonitrile

537. PFKFB3 inhibitor for use in neuroprotection, wherein a PFKFB3 inhibitor is 2-methyl-6-(3-methyl-1-benzofuran-5-yl)-N-[(1S)-1-{3-[2-(piperazin-1-yl)pyrimidin-5-yl]phenyl}ethyl]pyrimidin-4-amine

538. PFKFB3 inhibitor for use in neuroprotection, wherein a PFKFB3 inhibitor is (5-{3-[(1S)-1-{[6-(1,3-benzothiazol-6-yl)-2-methylpyrimidin-4-yl]amino}ethyl]phenyl}pyridin-3-yl)methanol

539. PFKFB3 inhibitor for use in neuroprotection, wherein a PFKFB3 inhibitor is 5-[3-(1-{[6-(1,3-benzothiazol-6-yl)-2-methylpyrimidin-4-yl]amino}ethyl)phenyl]pyridine-3-carboxamide

540. PFKFB3 inhibitor for use in neuroprotection, wherein a PFKFB3 inhibitor is N-(1-{3-[2-(dimethylamino)ethoxy]phenyl}ethyl)-2-methyl-6-(3-methyl-1-benzofuran-5-yl)pyrimidin-4-amine

541. PFKFB3 inhibitor for use in neuroprotection, wherein a PFKFB3 inhibitor is 6-(1,3-benzothiazol-6-yl)-2-methyl-N-(pentan-2-yl)pyrimidin-4-amine

542. PFKFB3 inhibitor for use in neuroprotection, wherein a PFKFB3 inhibitor is 6-(3,4-dimethoxyphenyl)-2-methyl-N-[(1R)-1,2,3,4-tetrahydronaphthalen-1-yl]pyrimidin-4-amine

543. PFKFB3 inhibitor for use in neuroprotection, wherein a PFKFB3 inhibitor is 6-(7-fluoro-3-methyl-1-benzofuran-5-yl)-2-methyl-N-[(1S)-1-[3-(1-methyl-1H-pyrazol-4-yl)phenyl]ethyl]pyrimidin-4-amine

544. PFKFB3 inhibitor for use in neuroprotection, wherein a PFKFB3 inhibitor is N-[(1S)-1-[3-(5-amino-6-chloropyridin-3-yl)phenyl]ethyl]-2-methyl-6-(3-methyl-1-benzofuran-5-yl)pyrimidin-4-amine

545. PFKFB3 inhibitor for use in neuroprotection, wherein a PFKFB3 inhibitor is 2-fluoro-4-(2-methyl-6-{[(1R)-1,2,3,4-tetrahydronaphthalen-1-yl]amino}pyrimidin-4-yl)benzamide

546. PFKFB3 inhibitor for use in neuroprotection, wherein a PFKFB3 inhibitor is 2-methyl-6-(3-methyl-1-benzofuran-5-yl)-N-[(1S)-1-{3-[6-(piperazin-1-yl)pyridin-3-yl]phenyl}ethyl]pyrimidin-4-amine

547. PFKFB3 inhibitor for use in neuroprotection, wherein a PFKFB3 inhibitor is 2-methyl-N-{1-[3-(1-methyl-1H-pyrazol-4-yl)phenyl]ethyl}-6-(4-methyl-3,4-dihydro-2H-1,4-benzoxazin-6-yl)pyrimidin-4-amine

548. PFKFB3 inhibitor for use in neuroprotection, wherein a PFKFB3 inhibitor is 6-(1,3-benzothiazol-6-yl)-2-methyl-N-(1-{3-[(1-methyl-1H-pyrazol-3-yl)methoxy]phenyl}ethyl)pyrimidin-4-amine

549. PFKFB3 inhibitor for use in neuroprotection, wherein a PFKFB3 inhibitor is N-{1-[4-fluoro-3-(1-methyl-1H-pyrazol-4-yl)phenyl]ethyl}-2-methyl-6-(3-methyl-1-benzofuran-5-yl)pyrimidin-4-amine

550. PFKFB3 inhibitor for use in neuroprotection, wherein a PFKFB3 inhibitor is 6-(1,3-benzothiazol-6-yl)-N-[(1S)-1-[3-(1-ethyl-1H-pyrazol-4-yl)phenyl]ethyl]-2-methylpyrimidin-4-amine

551. PFKFB3 inhibitor for use in neuroprotection, wherein a PFKFB3 inhibitor is 6-chloro-5′-[(1S)-1-{[2-methyl-6-(3-methyl-1-benzofuran-5-yl)pyrimidin-4-yl]amino}ethyl]-[3,3′-bipyridin]-5-amine

552. PFKFB3 inhibitor for use in neuroprotection, wherein a PFKFB3 inhibitor is 6-(1,3-benzothiazol-6-yl)-N-(1-{3-[(1,3-dimethyl-1H-pyrazol-5-yl)methoxy]phenyl}ethyl)-2-methylpyrimidin-4-amine

553. PFKFB3 inhibitor for use in neuroprotection, wherein a PFKFB3 inhibitor is 6-(1,3-benzothiazol-6-yl)-2-methyl-N-(2-methylbutyl)pyrimidin-4-amine

554. PFKFB3 inhibitor for use in neuroprotection, wherein a PFKFB3 inhibitor is 1-hydroxy-3-[(5-{3-[(1S)-1-{[2-methyl-6-(3-methyl-1-benzofuran-5-yl)pyrimidin-4-yl]amino}ethyl]phenyl}pyrimidin-2-yl)amino]propan-2-one

555. PFKFB3 inhibitor for use in neuroprotection, wherein a PFKFB3 inhibitor is 2-methyl-6-(3-methyl-1-benzofuran-5-yl)-N-[(1S)-1-{3-[6-(morpholin-4-yl)pyridin-3-yl]phenyl}ethyl]pyrimidin-4-amine

556. PFKFB3 inhibitor for use in neuroprotection, wherein a PFKFB3 inhibitor is 6-(1,3-benzothiazol-6-yl)-2-methyl-N-{1-[3-(1H-1,2,3-triazol-1-yl)phenyl]ethyl}pyrimidin-4-amine

557. PFKFB3 inhibitor for use in neuroprotection, wherein a PFKFB3 inhibitor is 1-(azetidin-1-yl)-2-[3-(1-{[6-(1,3-benzothiazol-6-yl)-2-methylpyrimidin-4-yl]amino}ethyl)phenoxy]ethan- -one

558. PFKFB3 inhibitor for use in neuroprotection, wherein a PFKFB3 inhibitor is N-(5-{[6-(1,3-benzothiazol-6-yl)-2-methylpyrimidin-4-yl]amino}-5,6,7,8-tetrahydronaphthalen-2-yl)acetamide

559. PFKFB3 inhibitor for use in neuroprotection, wherein a PFKFB3 inhibitor is N-{1-[3-(2-aminopyrimidin-5-yl)-5-fluorophenyl]ethyl}-2-methyl-6-(3-methyl-1-benzofuran-5-yl)pyrimidin-4-amine

560. PFKFB3 inhibitor for use in neuroprotection, wherein a PFKFB3 inhibitor is 2-[3-(1-{[6-(2-chloro-3-methoxyphenyl)-2-methylpyrimidin-4-yl]amino}ethyl)phenoxy]-N-methylacetamide

561. PFKFB3 inhibitor for use in neuroprotection, wherein a PFKFB3 inhibitor is 2-methyl-6-(3-methyl-1-benzofuran-5-yl)-N-[(1S)-1-[3-(6-methylpyridin-3-yl)phenyl]ethyl]pyrimidin-4-amine

562. PFKFB3 inhibitor for use in neuroprotection, wherein a PFKFB3 inhibitor is 6-(4-chloro-3,5-difluorophenyl)-2-methyl-N-{1-[3-(1-methyl-1H-pyrazol-4-yl)phenyl]ethyl}pyrimidin-4-amine

563. PFKFB3 inhibitor for use in neuroprotection, wherein a PFKFB3 inhibitor is 6-(3-ethyl-1-benzofuran-5-yl)-2-methyl-N-[(1R)-1,2,3,4-tetrahydronaphthalen-1-yl]pyrimidin-4-amine

564. PFKFB3 inhibitor for use in neuroprotection, wherein a PFKFB3 inhibitor is 6-(1-benzothiophen-2-yl)-2-methyl-N-[(1R)-1,2,3,4-tetrahydronaphthalen-1-yl]pyrimidin-4-amine

565. PFKFB3 inhibitor for use in neuroprotection, wherein a PFKFB3 inhibitor is 6-(1,3-benzothiazol-6-yl)-N-{1-[3-(3,5-dimethyl-1,2-oxazol-4-yl)phenyl]ethyl}-2-methylpyrimidin-4-amine

566. PFKFB3 inhibitor for use in neuroprotection, wherein a PFKFB3 inhibitor is N-{1-[3-(2-aminopyrimidin-5-yl)-4-fluorophenyl]ethyl}-2-methyl-6-(3-methyl-1-benzofuran-5-yl)pyrimidin-4-amine

567. PFKFB3 inhibitor for use in neuroprotection, wherein a PFKFB3 inhibitor is 2-chloro-5-(2-methyl-6-{[(1R)-1,2,3,4-tetrahydronaphthalen-1-yl]amino}pyrimidin-4-yl)benzonitrile

568. PFKFB3 inhibitor for use in neuroprotection, wherein a PFKFB3 inhibitor is 2-[3-(1-{[2-methyl-6-(3-methyl-1-benzofuran-5-yl)pyrimidin-4-yl]amino}ethyl)phenoxy]ethan-1-ol

569. PFKFB3 inhibitor for use in neuroprotection, wherein a PFKFB3 inhibitor is methyl 1-{2-[3-(1-{[2-methyl-6-(1-methyl-1H-indol-6-yl)pyrimidin-4-yl]amino}ethyl)phenoxy]acetyl}piperidine-4-carboxylate

570. PFKFB3 inhibitor for use in neuroprotection, wherein a PFKFB3 inhibitor is 2-methyl-6-(2-phenylethyl)-N-[(1R)-1,2,3,4-tetrahydronaphthalen-1-yl]pyrimidin-4-amine

571. PFKFB3 inhibitor for use in neuroprotection, wherein a PFKFB3 inhibitor is 6-(3-chloro-4-methylphenyl)-2-methyl-N-[(1R)-1,2,3,4-tetrahydronaphthalen-1-yl]pyrimidin-4-amine

572. PFKFB3 inhibitor for use in neuroprotection, wherein a PFKFB3 inhibitor is 6-(2,3-dihydro-1,4-benzodioxin-6-yl)-2-methyl-N-{1-[3-(1-methyl-1H-pyrazol-4-yl)phenyl]ethyl}pyrimidin-4-amine

573. PFKFB3 inhibitor for use in neuroprotection, wherein a PFKFB3 inhibitor is 6-(1-benzofuran-5-yl)-N-[(1S)-1-(3-methoxyphenyl)ethyl]-2-methylpyrimidin-4-amine

574. PFKFB3 inhibitor for use in neuroprotection, wherein a PFKFB3 inhibitor is N-{1-[3-(5-amino-6-chloropyridin-3-yl)-5-fluorophenyl]ethyl}-2-methyl-6-(3-methyl-1-benzofuran-5-yl)pyrimidin-4-amine

575. PFKFB3 inhibitor for use in neuroprotection, wherein a PFKFB3 inhibitor is 6-(1,3-benzothiazol-6-yl)-N-{1-[3-(4-chloropyridin-3-yl)phenyl]ethyl}-2-methylpyrimidin-4-amine

576. PFKFB3 inhibitor for use in neuroprotection, wherein a PFKFB3 inhibitor is N-[1-(3-aminophenyl)ethyl]-6-(1,3-benzothiazol-6-yl)-2-methylpyrimidin-4-amine

577. PFKFB3 inhibitor for use in neuroprotection, wherein a PFKFB3 inhibitor is N-[(1S)-1-{3-[2-(dimethylamino)pyrimidin-5-yl]phenyl}ethyl]-2-methyl-6-(3-methyl-1-benzofuran-5-yl)pyrimidin-4-amine

578. PFKFB3 inhibitor for use in neuroprotection, wherein a PFKFB3 inhibitor is 6-(1,3-benzothiazol-6-yl)-2-methyl-N-[(1S)-1-[3-(1-methyl-1H-pyrrol-2-yl)phenyl]ethyl]pyrimidin-4-amine

579. PFKFB3 inhibitor for use in neuroprotection, wherein a PFKFB3 inhibitor is 6-(2H-1,3-benzodioxol-5-yl)-2-methyl-N-[(1R)-1,2,3,4-tetrahydronaphthalen-1-yl]pyrimidin-4-amine

580. PFKFB3 inhibitor for use in neuroprotection, wherein a PFKFB3 inhibitor is 6-(1,3-benzothiazol-6-yl)-N-(1-{3-[(1-ethylpiperidin-3-yl)methoxy]phenyl}ethyl)-2-methylpyrimidin-4-amine

581. PFKFB3 inhibitor for use in neuroprotection, wherein a PFKFB3 inhibitor is 2-[(5-{3-[(1S)-1-{[2-methyl-6-(3-methyl-1-benzofuran-5-yl)pyrimidin-4-yl]amino}ethyl]phenyl}pyridin-2-yl)amino]ethan-1-ol

582. PFKFB3 inhibitor for use in neuroprotection, wherein a PFKFB3 inhibitor is 6-(1,3-benzothiazol-6-yl)-N-{1-[3-(2-fluoropyrimidin-5-yl)phenyl]ethyl}-2-methylpyrimidin-4-amine

583. PFKFB3 inhibitor for use in neuroprotection, wherein a PFKFB3 inhibitor is 6-(1,3-benzothiazol-6-yl)-N-(6-fluoro-3,4-dihydro-2H-1-benzopyran-4-yl)-2-methylpyrimidin-4-amine

584. PFKFB3 inhibitor for use in neuroprotection, wherein a PFKFB3 inhibitor is N-[(1S)-1-[3-(2-aminopyrimidin-5-yl)phenyl]ethyl]-6-(1,3-benzothiazol-6-yl)-2-methylpyrimidin-4-amine

585. PFKFB3 inhibitor for use in neuroprotection, wherein a PFKFB3 inhibitor is 2-[(5-{3-[(1S)-1-{[2-methyl-6-(3-methyl-1-benzofuran-5-yl)pyrimidin-4-yl]amino}ethyl]phenyl}pyrimidin-2-yl)amino]ethan-1-ol

586. PFKFB3 inhibitor for use in neuroprotection, wherein a PFKFB3 inhibitor is N,N-diethyl-2-[3-(1-{[2-methyl-6-(4-methyl-3,4-dihydro-2H-1,4-benzoxazin-6-yl)pyrimidin-4-yl]amino}ethyl)phenoxy]acetamide

587. PFKFB3 inhibitor for use in neuroprotection, wherein a PFKFB3 inhibitor is 3-(1-{[6-(1,3-benzothiazol-6-yl)-2-methylpyrimidin-4-yl]amino}ethyl)benzene-1-sulfonamide

588. PFKFB3 inhibitor for use in neuroprotection, wherein a PFKFB3 inhibitor is 2-methyl-6-(3-methyl-1-benzofuran-5-yl)-N-[(1S)-1-(4-methylphenyl)ethyl]pyrimidin-4-amine

589. PFKFB3 inhibitor for use in neuroprotection, wherein a PFKFB3 inhibitor is N-[(1S)-1-[3-(5-amino-6-chloropyridin-3-yl)phenyl]ethyl]-6-(7-fluoro-3-methyl-1-benzofuran-5-yl)-2-methylpyrimidin-4-amine

590. PFKFB3 inhibitor for use in neuroprotection, wherein a PFKFB3 inhibitor is 6-(1,3-benzothiazol-6-yl)-2-methyl-N-[1-(3-nitrophenyl)ethyl]pyrimidin-4-amine

591. PFKFB3 inhibitor for use in neuroprotection, wherein a PFKFB3 inhibitor is 3-{3-[(1S)-1-{[6-(1,3-benzothiazol-6-yl)-2-methylpyrimidin-4-yl]amino}ethyl]phenyl}-N-tert-butylprop-2-enamide

592. PFKFB3 inhibitor for use in neuroprotection, wherein a PFKFB3 inhibitor is N-[(1S)-1-[3-(2-aminopyrimidin-5-yl)phenyl]ethyl]-2-methyl-6-(1-methyl-1H-indol-6-yl)pyrimidin-4-amine

593. PFKFB3 inhibitor for use in neuroprotection, wherein a PFKFB3 inhibitor is 2-methyl-6-(1-methyl-2,3-dihydro-1H-indol-6-yl)-N-[(1R)-1,2,3,4-tetrahydronaphthalen-1-yl]pyrimidin-4-amine

594. PFKFB3 inhibitor for use in neuroprotection, wherein a PFKFB3 inhibitor is 3-(1-{[6-(1,3-benzothiazol-6-yl)-2-methylpyrimidin-4-yl]amino}ethyl)benzonitrile

595. PFKFB3 inhibitor for use in neuroprotection, wherein a PFKFB3 inhibitor is N-{1-[5-(2-aminopyrimidin-5-yl)pyridin-3-yl]ethyl}-6-(7-fluoro-3-methyl-1-benzofuran-5-yl)-2-methylpyrimidin-4-amine

596. PFKFB3 inhibitor for use in neuroprotection, wherein a PFKFB3 inhibitor is 2-methyl-6-(3-methyl-1-benzofuran-5-yl)-N-[(1R)-1,2,3,4-tetrahydronaphthalen-1-yl]pyrimidin-4-amine

597. PFKFB3 inhibitor for use in neuroprotection, wherein a PFKFB3 inhibitor is 6-(4-chloro-3-fluorophenyl)-2-methyl-N-[(1S)-1-[3-(1-methyl-1H-pyrazol-4-yl)phenyl]ethyl]pyrimidin-4-amine

598. PFKFB3 inhibitor for use in neuroprotection, wherein a PFKFB3 inhibitor is 6-(1,3-benzothiazol-6-yl)-N-{1-[4-fluoro-3-(1-methyl-1H-pyrazol-4-yl)phenyl]ethyl}-2-methylpyrimidin-4-amine

599. PFKFB3 inhibitor for use in neuroprotection, wherein a PFKFB3 inhibitor is 6-(2H-1,3-benzodioxol-5-yl)-N-[(1S)-1-(3-methoxyphenyl)ethyl]-2-methylpyrimidin-4-amine

600. PFKFB3 inhibitor for use in neuroprotection, wherein a PFKFB3 inhibitor is 6-(7-fluoro-1-benzofuran-5-yl)-N-[(1S)-1-(3-methoxyphenyl)ethyl]-2-methylpyrimidin-4-amine

601. PFKFB3 inhibitor for use in neuroprotection, wherein a PFKFB3 inhibitor is 6-(1,3-benzothiazol-6-yl)-2-methyl-N-{1-[3-(pyridin-3-yl)phenyl]ethyl}pyrimidin-4-amine

602. PFKFB3 inhibitor for use in neuroprotection, wherein a PFKFB3 inhibitor is 6-(1,3-benzothiazol-6-yl)-2-methyl-N-(1-{3-[(2-methyl-1,3-thiazol-4-yl)methoxy]phenyl}ethyl)pyrimidin-4-amine

603. PFKFB3 inhibitor for use in neuroprotection, wherein a PFKFB3 inhibitor is N-{1-[3-(6-aminopyridin-3-yl)phenyl]ethyl}-6-(1,3-benzothiazol-6-yl)-2-methylpyrimidin-4-amine

604. PFKFB3 inhibitor for use in neuroprotection, wherein a PFKFB3 inhibitor is 1-[4-(5-{3-[(1S)-1-{[2-methyl-6-(3-methyl-1-benzofuran-5-yl)pyrimidin-4-yl]amino}ethyl]phenyl}pyrimidin-2-yl)piperazin-1-yl]ethan-1-one

605. PFKFB3 inhibitor for use in neuroprotection, wherein a PFKFB3 inhibitor is 2-[(5-{3-[(1S)-1-{[2-methyl-6-(3-methyl-1-benzofuran-5-yl)pyrimidin-4-yl]amino}ethyl]phenyl}pyrimidin-2-yl)amino]acetic acid

606. PFKFB3 inhibitor for use in neuroprotection, wherein a PFKFB3 inhibitor is 6-(1,3-benzothiazol-6-yl)-2-methyl-N-(1-{3-[(1-methylpiperidin-3-yl)methoxy]phenyl}ethyl)pyrimidin-4-amine

607. PFKFB3 inhibitor for use in neuroprotection, wherein a PFKFB3 inhibitor is 6-(1-benzofuran-2-yl)-2-methyl-N-[(1R)-1,2,3,4-tetrahydronaphthalen-1-yl]pyrimidin-4-amine

608. PFKFB3 inhibitor for use in neuroprotection, wherein a PFKFB3 inhibitor is 2-[3-(1-{[2-methyl-6-(1-methyl-1H-indol-6-yl)pyrimidin-4-yl]amino}ethyl)phenoxy]acetonitrile

609. PFKFB3 inhibitor for use in neuroprotection, wherein a PFKFB3 inhibitor is 6-(1,3-benzothiazol-6-yl)-N-{1-[3-(6-methoxypyridin-3-yl)phenyl]ethyl}-2-methylpyrimidin-4-amine

610. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-1-(pyrrolidin-1-yl)ethan-1-one

611. PFKFB3 inhibitor for use in neuroprotection, wherein a PFKFB3 inhibitor is 2-methyl-6-(3-methyl-1-benzofuran-5-yl)-N-[(1R)-1-[3-(1-methyl-1H-pyrazol-4-yl)phenyl]ethyl]pyrimidin-4-amine

612. PFKFB3 inhibitor for use in neuroprotection, wherein a PFKFB3 inhibitor is 3-(1-{[6-(3-ethyl-1-benzofuran-5-yl)-2-methylpyrimidin-4-yl]amino}ethyl)phenol

613. PFKFB3 inhibitor for use in neuroprotection, wherein a PFKFB3 inhibitor is N-{1-[3-(furan-3-yl)phenyl]ethyl}-2-methyl-6-(3-methyl-1-benzofuran-5-yl)pyrimidin-4-amine

614. PFKFB3 inhibitor for use in neuroprotection, wherein a PFKFB3 inhibitor is 2-methyl-6-(quinolin-3-yl)-N-[(1R)-1,2,3,4-tetrahydronaphthalen-1-yl]pyrimidin-4-amine

615. PFKFB3 inhibitor for use in neuroprotection, wherein a PFKFB3 inhibitor is 6-(1,3-benzothiazol-6-yl)-2-methyl-N-(1-{3-[(5-methyl-1,2-oxazol-3-yl)methoxy]phenyl}ethyl)pyrimidin-4-amine

616. PFKFB3 inhibitor for use in neuroprotection, wherein a PFKFB3 inhibitor is 6-(1,3-benzothiazol-6-yl)-N-[(1S)-1-(3-methoxyphenyl)ethyl]-2-methylpyrimidin-4-amine

617. PFKFB3 inhibitor for use in neuroprotection, wherein a PFKFB3 inhibitor is N-[(1S)-1-(3-methoxyphenyl)ethyl]-2-methyl-6-(3-methyl-1-benzofuran-5-yl)pyrimidin-4-amine

618. PFKFB3 inhibitor for use in neuroprotection, wherein a PFKFB3 inhibitor is 2-methyl-6-(quinoxalin-6-yl)-N-[(1R)-1,2,3,4-tetrahydronaphthalen-1-yl]pyrimidin-4-amine

619. PFKFB3 inhibitor for use in neuroprotection, wherein a PFKFB3 inhibitor is 6-(1-ethyl-1H-indol-6-yl)-2-methyl-N-[(1R)-1,2,3,4-tetrahydronaphthalen-1-yl]pyrimidin-4-amine

620. PFKFB3 inhibitor for use in neuroprotection, wherein a PFKFB3 inhibitor is 6-(1,3-benzothiazol-6-yl)-2-methyl-N-[(1S)-1-[3-(1H-pyrazol-3-yl)phenyl]ethyl]pyrimidin-4-amine

621. PFKFB3 inhibitor for use in neuroprotection, wherein a PFKFB3 inhibitor is 2-methyl-6-{4-methyl-3-[(prop-2-yn-1-yl)amino]phenyl}-N-[(1R)-1,2,3,4-tetrahydronaphthalen-1-yl]pyrimidin-4-amine

622. PFKFB3 inhibitor for use in neuroprotection, wherein a PFKFB3 inhibitor is 6-(2-fluoro-3-methoxyphenyl)-2-methyl-N-[(1R)-1,2,3,4-tetrahydronaphthalen-1-yl]pyrimidin-4-amine

623. PFKFB3 inhibitor for use in neuroprotection, wherein a PFKFB3 inhibitor is 6-(1,3-benzothiazol-6-yl)-2-methyl-N-[(1S)-1-{3-[2-(methylamino)pyrimidin-5-yl]phenyl}ethyl]pyrimidin-4-amine

624. PFKFB3 inhibitor for use in neuroprotection, wherein a PFKFB3 inhibitor is tert-butyl 3-{[3-(1-{[2-methyl-6-(3-methyl-1-benzofuran-5-yl)pyrimidin-4-yl]amino}ethyl)phenoxy]methyl}piperidine-1-carboxylate

625. PFKFB3 inhibitor for use in neuroprotection, wherein a PFKFB3 inhibitor is N-[6-(1,3-benzothiazol-6-yl)-2-methylpyrimidin-4-yl]-1-methyl-1,2,3,4-tetrahydroquinolin-4-amine

626. PFKFB3 inhibitor for use in neuroprotection, wherein a PFKFB3 inhibitor is 2-methyl-6-(3-methyl-1-benzofuran-5-yl)-N-[(1S)-1-[5-(1-methyl-1H-pyrazol-5-yl)pyridin-3-yl]ethyl]pyrimidin-4-amine

627. PFKFB3 inhibitor for use in neuroprotection, wherein a PFKFB3 inhibitor is 6-(7-fluoro-1-benzofuran-5-yl)-2-methyl-N-{1-[3-(1-methyl-1H-pyrazol-4-yl)phenyl]ethyl}pyrimidin-4-amine

628. PFKFB3 inhibitor for use in neuroprotection, wherein a PFKFB3 inhibitor is 6-(4-ethyl-3,4-dihydro-2H-1,4-benzoxazin-6-yl)-2-methyl-N-[(1R)-1,2,3,4-tetrahydronaphthalen-1-yl]pyrimidin-4-amine

629. PFKFB3 inhibitor for use in neuroprotection, wherein a PFKFB3 inhibitor is 6-(1,3-benzothiazol-6-yl)-2-methyl-N-{1-[3-(5-methyl-1,2,4-oxadiazol-3-yl)phenyl]ethyl}pyrimidin-4-amine

630. PFKFB3 inhibitor for use in neuroprotection, wherein a PFKFB3 inhibitor is 6-(3-amino-4-chlorophenyl)-N-[(4R)-3,4-dihydro-2H-1-benzopyran-4-yl]-2-methylpyrimidin-4-amine

631. PFKFB3 inhibitor for use in neuroprotection, wherein a PFKFB3 inhibitor is ethyl 2-(4-{3-[(1S)-1-{[2-methyl-6-(3-methyl-1-benzofuran-5-yl)pyrimidin-4-yl]amino}ethyl]phenyl}-1H-pyrazol-1-yl)acetate

632. PFKFB3 inhibitor for use in neuroprotection, wherein a PFKFB3 inhibitor is 6-[3-(dimethylamino)phenyl]-2-methyl-N-[(1R)-1,2,3,4-tetrahydronaphthalen-1-yl]pyrimidin-4-amine

633. PFKFB3 inhibitor for use in neuroprotection, wherein a PFKFB3 inhibitor is 3-[(1R)-1-{[2-methyl-6-(3-methyl-1-benzofuran-5-yl)pyrimidin-4-yl]amino}ethyl]phenol

634. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-(2-methoxyethyl)acetamide

635. PFKFB3 inhibitor for use in neuroprotection, wherein a PFKFB3 inhibitor is 2-methyl-6-phenyl-N-[(1R)-1,2,3,4-tetrahydronaphthalen-1-yl]pyrimidin-4-amine

636. PFKFB3 inhibitor for use in neuroprotection, wherein a PFKFB3 inhibitor is 6-(1,3-benzothiazol-6-yl)-N-[(2-fluoro-5-methoxyphenyl)methyl]-2-methylpyrimidin-4-amine

637. PFKFB3 inhibitor for use in neuroprotection, wherein a PFKFB3 inhibitor is 2-methyl-6-(3-methyl-1-benzofuran-5-yl)-N-[(1S)-1-[3-(5-methylpyridin-3-yl)phenyl]ethyl]pyrimidin-4-amine

638. PFKFB3 inhibitor for use in neuroprotection, wherein a PFKFB3 inhibitor is 2-methyl-6-(1-methyl-1H-indazol-6-yl)-N-[(1R)-1,2,3,4-tetrahydronaphthalen-1-yl]pyrimidin-4-amine

639. PFKFB3 inhibitor for use in neuroprotection, wherein a PFKFB3 inhibitor is 3-{3-[(1S)-1-{[6-(1,3-benzothiazol-6-yl)-2-methylpyrimidin-4-yl]amino}ethyl]phenyl}-N-ethylprop-2-enamide

640. PFKFB3 inhibitor for use in neuroprotection, wherein a PFKFB3 inhibitor is N-[(4R)-3,4-dihydro-2H-1-benzopyran-4-yl]-2-methyl-6-(4-methyl-3,4-dihydro-2H-1,4-benzoxazin-6-yl)pyrimidin-4-amine

641. PFKFB3 inhibitor for use in neuroprotection, wherein a PFKFB3 inhibitor is 1-[3-(1-{[6-(1,3-benzothiazol-6-yl)-2-methylpyrimidin-4-yl]amino}ethyl)phenoxy]-2-methylpropan-2-ol

642. PFKFB3 inhibitor for use in neuroprotection, wherein a PFKFB3 inhibitor is 6-(1,3-benzothiazol-6-yl)-N-[(1S)-1-{3-[2-(4-ethylpiperazin-1-yl)pyrimidin-5-yl]phenyl}ethyl]-2-methylpyrimidin-4-amine

643. PFKFB3 inhibitor for use in neuroprotection, wherein a PFKFB3 inhibitor is 6-(1,3-benzothiazol-6-yl)-2-methyl-N-[(1S)-1-{3-[2-(piperidin-1-yl)pyrimidin-5-yl]phenyl}ethyl]pyrimidin-4-amine

644. PFKFB3 inhibitor for use in neuroprotection, wherein a PFKFB3 inhibitor is 6-(1,3-benzothiazol-6-yl)-N-(1-{3-[2-(1H-imidazol-1-yl)ethoxy]phenyl}ethyl)-2-methylpyrimidin-4-amine

645. PFKFB3 inhibitor for use in neuroprotection, wherein a PFKFB3 inhibitor is 6-(1,3-benzothiazol-6-yl)-N-[(2-chloro-6-fluoro-3-methylphenyl)methyl]-2-methylpyrimidin-4-amine

646. PFKFB3 inhibitor for use in neuroprotection, wherein a PFKFB3 inhibitor is 6-(3-amino-4-chlorophenyl)-2-methyl-N-[(1R)-1,2,3,4-tetrahydronaphthalen-1-yl]pyrimidin-4-amine

647. PFKFB3 inhibitor for use in neuroprotection, wherein a PFKFB3 inhibitor is 2-{3-[(1R)-1-{[2-methyl-6-(3-methyl-1-benzofuran-5-yl)pyrimidin-4-yl]amino}ethyl]phenoxy}-1-(morpholin-4-yl)ethan-1-one

648. PFKFB3 inhibitor for use in neuroprotection, wherein a PFKFB3 inhibitor is N-{1-[3-fluoro-5-(1-methyl-1H-pyrazol-4-yl)phenyl]ethyl}-2-methyl-6-(3-methyl-1-benzofuran-5-yl)pyrimidin-4-amine

649. PFKFB3 inhibitor for use in neuroprotection, wherein a PFKFB3 inhibitor is 6-(1,3-benzothiazol-6-yl)-N-{1-[3-(6-fluoro-2-methylpyridin-3-yl)phenyl]ethyl}-2-methylpyrimidin-4-amine

650. PFKFB3 inhibitor for use in neuroprotection, wherein a PFKFB3 inhibitor is 6-(1,3-benzothiazol-6-yl)-2-methyl-N-(1-{3-[5-(trifluoromethyl)pyridin-3-yl]phenyl}ethyl)pyrimidin-4-amine

651. PFKFB3 inhibitor for use in neuroprotection, wherein a PFKFB3 inhibitor is N-[(1S)-1-{3-[2-(4-fluoropiperidin-1-yl)pyrimidin-5-yl]phenyl}ethyl]-2-methyl-6-(3-methyl-1-benzofuran-5-yl)pyrimidin-4-amine

652. PFKFB3 inhibitor for use in neuroprotection, wherein a PFKFB3 inhibitor is 2-[4-(5-{3-[(1S)-1-{[2-methyl-6-(3-methyl-1-benzofuran-5-yl)pyrimidin-4-yl]amino}ethyl]phenyl}pyrimidin-2-yl)piperazin-1-yl]ethan-1-ol

653. PFKFB3 inhibitor for use in neuroprotection, wherein a PFKFB3 inhibitor is 4-(2-methyl-6-{[(1R)-1,2,3,4-tetrahydronaphthalen-1-yl]amino}pyrimidin-4-yl)phenol

654. PFKFB3 inhibitor for use in neuroprotection, wherein a PFKFB3 inhibitor is 2-methyl-6-(3-methyl-1-benzofuran-5-yl)-N-[(1S)-1-[3-(1H-pyrazol-4-yl)phenyl]ethyl]pyrimidin-4-amine

655. PFKFB3 inhibitor for use in neuroprotection, wherein a PFKFB3 inhibitor is 6-(7-fluoro-3-methyl-1-benzofuran-5-yl)-2-methyl-N-{1-[5-(1H-pyrazol-4-yl)pyridin-3-yl]ethyl}pyrimidin-4-amine

656. PFKFB3 inhibitor for use in neuroprotection, wherein a PFKFB3 inhibitor is N-[1-(3-fluorophenyl)ethyl]-2-methyl-6-(3-methyl-1-benzofuran-5-yl)pyrimidin-4-amine

657. PFKFB3 inhibitor for use in neuroprotection, wherein a PFKFB3 inhibitor is 2-{3-[(1S)-1-{[2-methyl-6-(3-methyl-1-benzofuran-5-yl)pyrimidin-4-yl]amino}ethyl]phenoxy}-1-(morpholin-4-yl)ethan-1-one

658. PFKFB3 inhibitor for use in neuroprotection, wherein a PFKFB3 inhibitor is 3-(1-{[6-(1,3-benzothiazol-6-yl)-2-methylpyrimidin-4-yl]amino}ethyl)phenol

659. PFKFB3 inhibitor for use in neuroprotection, wherein a PFKFB3 inhibitor is 6-(1,3-benzothiazol-6-yl)-N-[(1S)-1-{3-[6-(4-ethylpiperazin-1-yl)pyridin-3-yl]phenyl}ethyl]-2-methylpyrimidin-4-amine

660. PFKFB3 inhibitor for use in neuroprotection, wherein a PFKFB3 inhibitor is 6-(4-ethylphenyl)-2-methyl-N-{1-[3-(1-methyl-1H-pyrazol-4-yl)phenyl]ethyl}pyrimidin-4-amine

661. PFKFB3 inhibitor for use in neuroprotection, wherein a PFKFB3 inhibitor is N-[6-(1,3-benzothiazol-6-yl)-2-methylpyrimidin-4-yl]-1,2,3,4-tetrahydroquinolin-4-amine

662. PFKFB3 inhibitor for use in neuroprotection, wherein a PFKFB3 inhibitor is 6-(1,3-benzothiazol-6-yl)-N-(3,4-dihydro-2H-1-benzopyran-4-yl)-2-methylpyrimidin-4-amine

663. PFKFB3 inhibitor for use in neuroprotection, wherein a PFKFB3 inhibitor is N-[(1S)-1-(4-methoxyphenyl)ethyl]-2-methyl-6-(3-methyl-1-benzofuran-5-yl)pyrimidin-4-amine

664. PFKFB3 inhibitor for use in neuroprotection, wherein a PFKFB3 inhibitor is 2-methyl-6-(3-methyl-1-benzofuran-5-yl)-N-[(1S)-1-[3-(1-methyl-1H-pyrazol-4-yl)phenyl]ethyl]pyrimidin-4-amine

665. PFKFB3 inhibitor for use in neuroprotection, wherein a PFKFB3 inhibitor is 2-methyl-6-(3-methyl-1-benzofuran-5-yl)-N-[(1S)-1-{3-[2-(4-methylpiperazin-1-yl)pyrimidin-5-yl]phenyl}ethyl]pyrimidin-4-amine

666. PFKFB3 inhibitor for use in neuroprotection, wherein a PFKFB3 inhibitor is 6-(4-methoxyphenyl)-2-methyl-N-{1-[3-(1-methyl-1H-pyrazol-4-yl)phenyl]ethyl}pyrimidin-4-amine

667. PFKFB3 inhibitor for use in neuroprotection, wherein a PFKFB3 inhibitor is 6-(3-fluorophenyl)-2-methyl-N-{1-[3-(1-methyl-1H-pyrazol-4-yl)phenyl]ethyl}pyrimidin-4-amine

668. PFKFB3 inhibitor for use in neuroprotection, wherein a PFKFB3 inhibitor is 6-(1,3-benzothiazol-6-yl)-N-{1-[3-(methanesulfonylmethoxy)phenyl]ethyl}-2-methylpyrimidin-4-amine

669. PFKFB3 inhibitor for use in neuroprotection, wherein a PFKFB3 inhibitor is 3-[2-methyl-6-({1-[3-(1-methyl-1H-pyrazol-4-yl)phenyl]ethyl}amino)pyrimidin-4-yl]phenol

670. PFKFB3 inhibitor for use in neuroprotection, wherein a PFKFB3 inhibitor is 6-(1H-indol-5-yl)-2-methyl-N-{1-[3-(1-methyl-1H-pyrazol-4-yl)phenyl]ethyl}pyrimidin-4-amine

671. PFKFB3 inhibitor for use in neuroprotection, wherein a PFKFB3 inhibitor is 7-(2-methyl-6-{[(1R)-1,2,3,4-tetrahydronaphthalen-1-yl]amino}pyrimidin-4-yl)naphthalen-1-ol

672. PFKFB3 inhibitor for use in neuroprotection, wherein a PFKFB3 inhibitor is 2-{[5-(6-{[(4R)-3,4-dihydro-2H-1-benzopyran-4-yl]amino}-2-methylpyrimidin-4-yl)-2-methylphenyl]amino}acetonitrile

673. PFKFB3 inhibitor for use in neuroprotection, wherein a PFKFB3 inhibitor is 2-methyl-6-(3-methyl-1-benzofuran-5-yl)-N-[(1S)-1-{6′-methyl-[3,3′-bipyridin]-5-yl}ethyl]pyrimidin-4-amine

674. PFKFB3 inhibitor for use in neuroprotection, wherein a PFKFB3 inhibitor is 6-(1,3-benzoxazol-6-yl)-2-methyl-N-[(1R)-1,2,3,4-tetrahydronaphthalen-1-yl]pyrimidin-4-amine

675. PFKFB3 inhibitor for use in neuroprotection, wherein a PFKFB3 inhibitor is 6-(1,3-benzothiazol-6-yl)-N-[(1S)-1-[3-(6-fluoropyridin-3-yl)phenyl]ethyl]-2-methylpyrimidin-4-amine

676. PFKFB3 inhibitor for use in neuroprotection, wherein a PFKFB3 inhibitor is (2S)-3-[(5-{3-[(1S)-1-{[2-methyl-6-(3-methyl-1-benzofuran-5-yl)pyrimidin-4-yl]amino}ethyl]phenyl}pyrimidin-2-yl)amino]propane-1,2-diol

677. PFKFB3 inhibitor for use in neuroprotection, wherein a PFKFB3 inhibitor is 6-[4-chloro-3-(dimethylamino)phenyl]-2-methyl-N-[(1R)-1,2,3,4-tetrahydronaphthalen-1-yl]pyrimidin-4-amine

678. PFKFB3 inhibitor for use in neuroprotection, wherein a PFKFB3 inhibitor is 2-methyl-6-(3-methyl-1-benzofuran-5-yl)-N-{1-[3-(thiophen-3-yl)phenyl]ethyl}pyrimidin-4-amine

679. PFKFB3 inhibitor for use in neuroprotection, wherein a PFKFB3 inhibitor is N-[(1S)-1-[3-(5-fluoropyridin-3-yl)phenyl]ethyl]-2-methyl-6-(3-methyl-1-benzothiophen-5-yl)pyrimidin-4-amine

680. PFKFB3 inhibitor for use in neuroprotection, wherein a PFKFB3 inhibitor is 6-(1-benzofuran-5-yl)-N-(1-{3-[(1,3-dimethyl-1H-pyrazol-5-yl)methoxy]phenyl}ethyl)-2-methylpyrimidin-4-amine

681. PFKFB3 inhibitor for use in neuroprotection, wherein a PFKFB3 inhibitor is 2-methyl-6-(3-methyl-1-benzofuran-5-yl)-N-(1-{3-[(piperidin-3-yl)methoxy]phenyl}ethyl)pyrimidin-4-amine

682. PFKFB3 inhibitor for use in neuroprotection, wherein a PFKFB3 inhibitor is 6-(4-chloro-3-fluorophenyl)-2-methyl-N-[(1R)-1,2,3,4-tetrahydronaphthalen-1-yl]pyrimidin-4-amine

683. PFKFB3 inhibitor for use in neuroprotection, wherein a PFKFB3 inhibitor is 1-(5-{3-[(1S)-1-{[2-methyl-6-(3-methyl-1-benzofuran-5-yl)pyrimidin-4-yl]amino}ethyl]phenyl}pyridin-3-yl)ethan-1-ol

684. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-[2-(dimethylamino)ethyl]acetamide

685. PFKFB3 inhibitor for use in neuroprotection, wherein a PFKFB3 inhibitor is N-{1-[3-(2-aminopyrimidin-5-yl)phenyl]ethyl}-6-(1,3-benzothiazol-6-yl)-2-methylpyrimidin-4-amine

686. PFKFB3 inhibitor for use in neuroprotection, wherein a PFKFB3 inhibitor is 2-{2-[4-(5-{3-[(1S)-1-{[2-methyl-6-(3-methyl-1-benzofuran-5-yl)pyrimidin-4-yl]amino}ethyl]phenyl}pyrimidin-2-yl)piperazin-1-yl]ethoxy}ethan-1-ol

687. PFKFB3 inhibitor for use in neuroprotection, wherein a PFKFB3 inhibitor is ethyl 2-(4-{3-[(1S)-1-{[6-(1,3-benzothiazol-6-yl)-2-methylpyrimidin-4-yl]amino}ethyl]phenyl}-1H-pyrazol-1-yl)acetate

688. PFKFB3 inhibitor for use in neuroprotection, wherein a PFKFB3 inhibitor is N-[(1S)-1-{3-[2-(diethylamino)pyrimidin-5-yl]phenyl}ethyl]-2-methyl-6-(3-methyl-1-benzofuran-5-yl)pyrimidin-4-amine

689. PFKFB3 inhibitor for use in neuroprotection, wherein a PFKFB3 inhibitor is N-[(1S)-1-[3-(6-chloro-5-methylpyridin-3-yl)phenyl]ethyl]-2-methyl-6-(3-methyl-1-benzofuran-5-yl)pyrimidin-4-amine

690. PFKFB3 inhibitor for use in neuroprotection, wherein a PFKFB3 inhibitor is (5-{3-[(1S)-1-{[2-methyl-6-(3-methyl-1-benzofuran-5-yl)pyrimidin-4-yl]amino}ethyl]phenyl}pyridin-3-yl)methanol

691. PFKFB3 inhibitor for use in neuroprotection, wherein a PFKFB3 inhibitor is 6-(3-ethyl-1-benzofuran-5-yl)-2-methyl-N-{1-[3-(1-methyl-1H-pyrazol-4-yl)phenyl]ethyl}pyrimidin-4-amine

692. PFKFB3 inhibitor for use in neuroprotection, wherein a PFKFB3 inhibitor is 3-(2-methyl-6-{[(1R)-1,2,3,4-tetrahydronaphthalen-1-yl]amino}pyrimidin-4-yl)benzonitrile

693. PFKFB3 inhibitor for use in neuroprotection, wherein a PFKFB3 inhibitor is 6-(1,3-benzothiazol-6-yl)-2-methyl-N-[(1S)-1-{3-[6-(morpholin-4-yl)pyridin-3-yl]phenyl}ethyl]pyrimidin-4-amine

694. PFKFB3 inhibitor for use in neuroprotection, wherein a PFKFB3 inhibitor is 6-(1,3-benzothiazol-6-yl)-N-{1-[3-(2-{[2-(dimethylamino)ethyl]amino}pyrimidin-5-yl)phenyl]ethyl}-2-methylpyrimidin-4-amine

695. PFKFB3 inhibitor for use in neuroprotection, wherein a PFKFB3 inhibitor is N-[(1S)-1-[3-(2-chloropyrimidin-5-yl)phenyl]ethyl]-2-methyl-6-(3-methyl-1-benzofuran-5-yl)pyrimidin-4-amine

696. PFKFB3 inhibitor for use in neuroprotection, wherein a PFKFB3 inhibitor is 6-(4-chloro-3-fluorophenyl)-2-methyl-N-{1-[5-(1-methyl-1H-pyrazol-4-yl)pyridin-3-yl]ethyl}pyrimidin-4-amine

697. PFKFB3 inhibitor for use in neuroprotection, wherein a PFKFB3 inhibitor is ethyl 5-[3-(1-{[6-(1,3-benzothiazol-6-yl)-2-methylpyrimidin-4-yl]amino}ethyl)phenyl]pyridine-3-carboxylate

698. PFKFB3 inhibitor for use in neuroprotection, wherein a PFKFB3 inhibitor is 6-(3-methoxy-4-methylphenyl)-2-methyl-N-[(1R)-1,2,3,4-tetrahydronaphthalen-1-yl]pyrimidin-4-amine

699. PFKFB3 inhibitor for use in neuroprotection, wherein a PFKFB3 inhibitor is (5-{3-[(1S)-1-{[6-(1,3-benzothiazol-6-yl)-2-methylpyrimidin-4-yl]amino}ethyl]phenyl}pyridin-2-yl)methanol

700. PFKFB3 inhibitor for use in neuroprotection, wherein a PFKFB3 inhibitor is 2-{4-[3-(1-{[6-(1,3-benzothiazol-6-yl)-2-methylpyrimidin-4-yl]amino}ethyl)phenyl]-1H-pyrazol-1-yl}acetic acid

701. PFKFB3 inhibitor for use in neuroprotection, wherein a PFKFB3 inhibitor is N-[(1S)-1-(3-bromophenyl)ethyl]-2-methyl-6-(3-methyl-1-benzofuran-5-yl)pyrimidin-4-amine

702. PFKFB3 inhibitor for use in neuroprotection, wherein a PFKFB3 inhibitor is 6-(1,3-benzothiazol-6-yl)-N-[(1S)-1-{3-[2-(ethylamino)pyrimidin-5-yl]phenyl}ethyl]-2-methylpyrimidin-4-amine

703. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]propanamide

704. PFKFB3 inhibitor for use in neuroprotection, wherein a PFKFB3 inhibitor is 2-{[2-chloro-5-(2-methyl-6-{[(1R)-1,2,3,4-tetrahydronaphthalen-1-yl]amino}pyrimidin-4-yl)phenyl]amino}acetonitrile

705. PFKFB3 inhibitor for use in neuroprotection, wherein a PFKFB3 inhibitor is 2-methyl-6-(4-methyl-3,4-dihydro-2H-1,4-benzoxazin-6-yl)-N-[(1R)-1,2,3,4-tetrahydronaphthalen-1-yl]pyrimidin-4-amine

706. PFKFB3 inhibitor for use in neuroprotection, wherein a PFKFB3 inhibitor is 1-[3-(1-{[6-(1,3-benzothiazol-6-yl)-2-methylpyrimidin-4-yl]amino}ethyl)phenoxy]propan-2-ol

707. PFKFB3 inhibitor for use in neuroprotection, wherein a PFKFB3 inhibitor is 6-(1-benzothiophen-5-yl)-N-[(4R)-3,4-dihydro-2H-1-benzopyran-4-yl]-2-methylpyrimidin-4-amine

708. PFKFB3 inhibitor for use in neuroprotection, wherein a PFKFB3 inhibitor is 2-methyl-6-[4-(methylamino)phenyl]-N-[(1R)-1,2,3,4-tetrahydronaphthalen-1-yl]pyrimidin-4-amine

709. PFKFB3 inhibitor for use in neuroprotection, wherein a PFKFB3 inhibitor is 6-(1,3-benzothiazol-6-yl)-N-[(1S)-1-[3-(6-chloro-5-methylpyridin-3-yl)phenyl]ethyl]-2-methylpyrimidin-4-amine

710. PFKFB3 inhibitor for use in neuroprotection, wherein a PFKFB3 inhibitor is N-[(1S)-1-[3-(5-aminopyridin-3-yl)phenyl]ethyl]-6-(4-chloro-3-fluorophenyl)-2-methylpyrimidin-4-amine

711. PFKFB3 inhibitor for use in neuroprotection, wherein a PFKFB3 inhibitor is ethyl 5-{3-[(1S)-1-{[2-methyl-6-(3-methyl-1-benzofuran-5-yl)pyrimidin-4-yl]amino}ethyl]phenyl}pyridine-3-carboxylate

712. PFKFB3 inhibitor for use in neuroprotection, wherein a PFKFB3 inhibitor is 6-(1-benzothiophen-5-yl)-2-methyl-N-{1-[3-(1-methyl-1H-pyrazol-4-yl)phenyl]ethyl}pyrimidin-4-amine

713. PFKFB3 inhibitor for use in neuroprotection, wherein a PFKFB3 inhibitor is N-ethyl-2-[3-(1-{[2-methyl-6-(1-methyl-1H-indol-6-yl)pyrimidin-4-yl]amino}ethyl)phenoxy]acetamide

714. PFKFB3 inhibitor for use in neuroprotection, wherein a PFKFB3 inhibitor is 6-(1-benzofuran-5-yl)-2-methyl-N-[(1R)-1,2,3,4-tetrahydronaphthalen-1-yl]pyrimidin-4-amine

715. PFKFB3 inhibitor for use in neuroprotection, wherein a PFKFB3 inhibitor is 6-(1H-indol-5-yl)-2-methyl-N-[(1R)-1,2,3,4-tetrahydronaphthalen-1-yl]pyrimidin-4-amine

716. PFKFB3 inhibitor for use in neuroprotection, wherein a PFKFB3 inhibitor is 5-{3-[(1S)-1-{[2-methyl-6-(3-methyl-1-benzofuran-5-yl)pyrimidin-4-yl]amino}ethyl]phenyl}pyridine-3-carboxamide

717. PFKFB3 inhibitor for use in neuroprotection, wherein a PFKFB3 inhibitor is 3-[3-(1-{[6-(1,3-benzothiazol-6-yl)-2-methylpyrimidin-4-yl]amino}ethyl)phenoxy]propan-1-ol

718. PFKFB3 inhibitor for use in neuroprotection, wherein a PFKFB3 inhibitor is 2-methyl-6-(3-methyl-1-benzofuran-5-yl)-N-[(1S)-1-[3-(2-{[(oxolan-2-yl)methyl]amino}pyrimidin-5-yl)phenyl]ethyl]pyrimidin-4-amine

719. PFKFB3 inhibitor for use in neuroprotection, wherein a PFKFB3 inhibitor is N-(1-{3-[(1,3-dimethyl-1H-pyrazol-5-yl)methoxy]phenyl}ethyl)-2-methyl-6-(1-methyl-1H-indol-6-yl)pyrimidin-4-amine

720. PFKFB3 inhibitor for use in neuroprotection, wherein a PFKFB3 inhibitor is 6-(1,3-benzothiazol-6-yl)-2-methyl-N-{1-[3-(1H-1,2,3,4-tetrazol-1-yl)phenyl]ethyl}pyrimidin-4-amine

721. PFKFB3 inhibitor for use in neuroprotection, wherein a PFKFB3 inhibitor is 6-(1,3-benzothiazol-6-yl)-N-(3,4-dihydro-1H-2-benzothiopyran-4-yl)-2-methylpyrimidin-4-amine

722. PFKFB3 inhibitor for use in neuroprotection, wherein a PFKFB3 inhibitor is N-(1-{3-[2-(1H-imidazol-1-yl)ethoxy]phenyl}ethyl)-2-methyl-6-(3-methyl-1-benzofuran-5-yl)pyrimidin-4-amine

723. PFKFB3 inhibitor for use in neuroprotection, wherein a PFKFB3 inhibitor is 2-methyl-N-{1-[3-(1-methyl-1H-pyrazol-4-yl)phenyl]ethyl}-6-(3,4,5-trifluorophenyl)pyrimidin-4-amine

724. PFKFB3 inhibitor for use in neuroprotection, wherein a PFKFB3 inhibitor is 1-(5-{3-[(1S)-1-{[6-(1,3-benzothiazol-6-yl)-2-methylpyrimidin-4-yl]amino}ethyl]phenyl}pyrimidin-2-yl)piperidin-4-ol

725. PFKFB3 inhibitor for use in neuroprotection, wherein a PFKFB3 inhibitor is N-[(1S)-1-[3-(5-aminopyridin-3-yl)phenyl]ethyl]-6-(1,3-benzothiazol-6-yl)-2-methylpyrimidin-4-amine

726. PFKFB3 inhibitor for use in neuroprotection, wherein a PFKFB3 inhibitor is N-[1-(2-fluoro-3-methoxyphenyl)ethyl]-2-methyl-6-(3-methyl-1-benzofuran-5-yl)pyrimidin-4-amine

727. PFKFB3 inhibitor for use in neuroprotection, wherein a PFKFB3 inhibitor is 6-(1,3-benzothiazol-6-yl)-2-methyl-N-[(1R)-1,2,3,4-tetrahydronaphthalen-1-yl]pyrimidin-4-amine

728. PFKFB3 inhibitor for use in neuroprotection, wherein a PFKFB3 inhibitor is 6-(1,3-benzothiazol-6-yl)-N-[(1-cyclopentanecarbonylpiperidin-3-yl)methyl]-2-methylpyrimidin-4-amine

729. PFKFB3 inhibitor for use in neuroprotection, wherein a PFKFB3 inhibitor is 1-[4-(5-{3-[(1S)-1-{[2-methyl-6-(3-methyl-1-benzothiophen-5-yl)pyrimidin-4-yl]amino}ethyl]phenyl}pyrimidin-2-yl)piperazin-1-yl]ethan-1-one

730. PFKFB3 inhibitor for use in neuroprotection, wherein a PFKFB3 inhibitor is N-[1-(2-fluorophenyl)ethyl]-2-methyl-6-(3-methyl-1-benzofuran-5-yl)pyrimidin-4-amine

731. PFKFB3 inhibitor for use in neuroprotection, wherein a PFKFB3 inhibitor is 6-(1,3-benzothiazol-6-yl)-2-methyl-N-(1-{3-[(3-methyl-1,2-oxazol-5-yl)methoxy]phenyl}ethyl)pyrimidin-4-amine

732. PFKFB3 inhibitor for use in neuroprotection, wherein a PFKFB3 inhibitor is 2-chloro-5-(1-{[2-methyl-6-(3-methyl-1-benzofuran-5-yl)pyrimidin-4-yl]amino}ethyl)phenol

733. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N,N-diethylpropanamide

734. PFKFB3 inhibitor for use in neuroprotection, wherein a PFKFB3 inhibitor is N-[(1S)-1-[3-(2-{[(furan-2-yl)methyl]amino}pyrimidin-5-yl)phenyl]ethyl]-2-methyl-6-(3-methyl-1-benzofuran-5-yl)pyrimidin-4-amine

735. PFKFB3 inhibitor for use in neuroprotection, wherein a PFKFB3 inhibitor is 2-[3-(1-{[2-methyl-6-(1-methyl-1H-indol-2-yl)pyrimidin-4-yl]amino}ethyl)phenoxy]acetamide

736. PFKFB3 inhibitor for use in neuroprotection, wherein a PFKFB3 inhibitor is 2-[3-({[6-(1,3-benzothiazol-6-yl)-2-methylpyrimidin-4-yl]amino}methyl)-4-fluorophenoxy]-1-(morpholin-4-yl)ethan-1-one

737. PFKFB3 inhibitor for use in neuroprotection, wherein a PFKFB3 inhibitor is 6-(2-fluoro-3-methoxyphenyl)-N-[(1S)-1-(3-methoxyphenyl)ethyl]-2-methylpyrimidin-4-amine

738. PFKFB3 inhibitor for use in neuroprotection, wherein a PFKFB3 inhibitor is 6-(1,3-benzothiazol-6-yl)-N-{1-[3-(2-fluoroethoxy)phenyl]ethyl}-2-methylpyrimidin-4-amine

739. PFKFB3 inhibitor for use in neuroprotection, wherein a PFKFB3 inhibitor is 6-(1,3-benzothiazol-6-yl)-2-methyl-N-[(1S)-1-{3-[2-(4-methylpiperazin-1-yl)pyrimidin-5-yl]phenyl}ethyl]pyrimidin-4-amine

740. PFKFB3 inhibitor for use in neuroprotection, wherein a PFKFB3 inhibitor is 6-(1,3-benzothiazol-6-yl)-2-methyl-N-{1-[3-(2-methylpyridin-4-yl)phenyl]ethyl}pyrimidin-4-amine

741. PFKFB3 inhibitor for use in neuroprotection, wherein a PFKFB3 inhibitor is 3-[3-(1-{[6-(1,3-benzothiazol-6-yl)-2-methylpyrimidin-4-yl]amino}ethyl)phenoxy]propane-1,2-diol

742. PFKFB3 inhibitor for use in neuroprotection, wherein a PFKFB3 inhibitor is 6-(4-fluoro-3-methylphenyl)-2-methyl-N-{1-[3-(1-methyl-1H-pyrazol-4-yl)phenyl]ethyl}pyrimidin-4-amine

743. PFKFB3 inhibitor for use in neuroprotection, wherein a PFKFB3 inhibitor is N-[(1S)-1-(3-methoxyphenyl)ethyl]-2-methyl-6-(3-methylphenyl)pyrimidin-4-amine

744. PFKFB3 inhibitor for use in neuroprotection, wherein a PFKFB3 inhibitor is N-[(1S)-1-[3-(6-fluoro-5-methylpyridin-3-yl)phenyl]ethyl]-2-methyl-6-(3-methyl-1-benzofuran-5-yl)pyrimidin-4-amine

745. PFKFB3 inhibitor for use in neuroprotection, wherein a PFKFB3 inhibitor is 2-[3-(1-{[2-methyl-6-(3-methyl-1-benzofuran-5-yl)pyrimidin-4-yl]amino}ethyl)phenoxy]-1-(morpholin-4-yl)ethan-1-one

746. PFKFB3 inhibitor for use in neuroprotection, wherein a PFKFB3 inhibitor is 2-methyl-6-(naphthalen-2-yl)-N-[(1R)-1,2,3,4-tetrahydronaphthalen-1-yl]pyrimidin-4-amine

747. PFKFB3 inhibitor for use in neuroprotection, wherein a PFKFB3 inhibitor is 6-(3-chlorophenyl)-N-[(1S)-1-(3-methoxyphenyl)ethyl]-2-methylpyrimidin-4-amine

748. PFKFB3 inhibitor for use in neuroprotection, wherein a PFKFB3 inhibitor is N-[(1S)-1-{6′-fluoro-[3,3′-bipyridin]-5-yl}ethyl]-2-methyl-6-(3-methyl-1-benzofuran-5-yl)pyrimidin-4-amine

749. PFKFB3 inhibitor for use in neuroprotection, wherein a PFKFB3 inhibitor is N-[1-(5-bromopyridin-3-yl)ethyl]-2-methyl-6-(3-methyl-1-benzofuran-5-yl)pyrimidin-4-amine

750. PFKFB3 inhibitor for use in neuroprotection, wherein a PFKFB3 inhibitor is 4-[3-(1-{[6-(1,3-benzothiazol-6-yl)-2-methylpyrimidin-4-yl]amino}ethyl)phenoxy]-N,N-dimethylbutanamide

751. PFKFB3 inhibitor for use in neuroprotection, wherein a PFKFB3 inhibitor is N-[(1R)-1-(3-methoxyphenyl)ethyl]-2-methyl-6-(3-methyl-1-benzofuran-5-yl)pyrimidin-4-amine

752. PFKFB3 inhibitor for use in neuroprotection, wherein a PFKFB3 inhibitor is 1-(5-{3-[(1S)-1-{[2-methyl-6-(3-methyl-1-benzofuran-5-yl)pyrimidin-4-yl]amino}ethyl]phenyl}pyrimidin-2-yl)pyrrolidin-3-ol

753. PFKFB3 inhibitor for use in neuroprotection, wherein a PFKFB3 inhibitor is 3-(1-{[6-(1,3-benzothiazol-6-yl)-2-methylpyrimidin-4-yl]amino}ethyl)-N-(but-2-yn-1-yl)benzene-1-sulfonamide

754. PFKFB3 inhibitor for use in neuroprotection, wherein a PFKFB3 inhibitor is 3-(1-{[2-methyl-6-(3-methyl-1-benzofuran-5-yl)pyrimidin-4-yl]amino}ethyl)phenol

755. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]ethan-1-ol

756. PFKFB3 inhibitor for use in neuroprotection, wherein a PFKFB3 inhibitor is 2-[3-(1-{[6-(3-methoxyphenyl)-2-methylpyrimidin-4-yl]amino}ethyl)phenoxy]acetonitrile

757. PFKFB3 inhibitor for use in neuroprotection, wherein a PFKFB3 inhibitor is 6-(2,4-dichlorophenyl)-2-methyl-N-{1-[3-(1-methyl-1H-pyrazol-4-yl)phenyl]ethyl}pyrimidin-4-amine

758. PFKFB3 inhibitor for use in neuroprotection, wherein a PFKFB3 inhibitor is 6-(3-methoxyphenyl)-2-methyl-N-{1-[3-(1-methyl-1H-pyrazol-4-yl)phenyl]ethyl}pyrimidin-4-amine

759. PFKFB3 inhibitor for use in neuroprotection, wherein a PFKFB3 inhibitor is N-[1-(3-aminophenyl)ethyl]-2-methyl-6-(3-methyl-1-benzofuran-5-yl)pyrimidin-4-amine

760. PFKFB3 inhibitor for use in neuroprotection, wherein a PFKFB3 inhibitor is 2-methyl-6-(3-methyl-1-benzofuran-5-yl)-N-[1-(3-methylphenyl)ethyl]pyrimidin-4-amine

761. PFKFB3 inhibitor for use in neuroprotection, wherein a PFKFB3 inhibitor is N-[(1S)-1-[3-(6-fluoropyridin-3-yl)phenyl]ethyl]-2-methyl-6-(3-methyl-1-benzothiophen-5-yl)pyrimidin-4-amine

762. PFKFB3 inhibitor for use in neuroprotection, wherein a PFKFB3 inhibitor is 6-(1,3-benzothiazol-6-yl)-2-methyl-N-(3-methylbutyl)pyrimidin-4-amine

763. PFKFB3 inhibitor for use in neuroprotection, wherein a PFKFB3 inhibitor is 2-methyl-N-[(1R)-1,2,3,4-tetrahydronaphthalen-1-yl]-6-[4-(trifluoromethyl)phenyl]pyrimidin-4-amine

764. PFKFB3 inhibitor for use in neuroprotection, wherein a PFKFB3 inhibitor is 6-(3,4-dichlorophenyl)-2-methyl-N-[(1R)-1,2,3,4-tetrahydronaphthalen-1-yl]pyrimidin-4-amine

765. PFKFB3 inhibitor for use in neuroprotection, wherein a PFKFB3 inhibitor is 1-(5-{3-[(1S)-1-{[2-methyl-6-(3-methyl-1-benzofuran-5-yl)pyrimidin-4-yl]amino}ethyl]phenyl}pyridin-2-yl)piperidin-4-ol

766. PFKFB3 inhibitor for use in neuroprotection, wherein a PFKFB3 inhibitor is 6-(1,3-benzothiazol-6-yl)-N-[(1S)-1-[3-(2-{[3-(dimethylamino)propyl]amino}pyrimidin-5-yl)phenyl]ethyl]-2-methylpyrimidin-4-amine

767. PFKFB3 inhibitor for use in neuroprotection, wherein a PFKFB3 inhibitor is 2-methyl-6-(3-methyl-1-benzofuran-5-yl)-N-{1-[3-(4H-1,2,4-triazol-4-yl)phenyl]ethyl}pyrimidin-4-amine

768. PFKFB3 inhibitor for use in neuroprotection, wherein a PFKFB3 inhibitor is 2-methyl-6-(3-methyl-1-benzofuran-5-yl)-N-[(1S)-1-[3-(1-methyl-1H-pyrrol-2-yl)phenyl]ethyl]pyrimidin-4-amine

769. PFKFB3 inhibitor for use in neuroprotection, wherein a PFKFB3 inhibitor is 2-methyl-6-(3-methyl-1-benzofuran-5-yl)-N-[(1S)-1-phenylethyl]pyrimidin-4-amine

770. PFKFB3 inhibitor for use in neuroprotection, wherein a PFKFB3 inhibitor is N-[(1S)-1-{5′-fluoro-[3,3′-bipyridin]-5-yl}ethyl]-2-methyl-6-(3-methyl-1-benzofuran-5-yl)pyrimidin-4-amine

771. PFKFB3 inhibitor for use in neuroprotection, wherein a PFKFB3 inhibitor is 6-[4-chloro-3-(methylamino)phenyl]-2-methyl-N-[(1R)-1,2,3,4-tetrahydronaphthalen-1-yl]pyrimidin-4-amine

772. PFKFB3 inhibitor for use in neuroprotection, wherein a PFKFB3 inhibitor is 2-methyl-6-(3-methyl-1-benzofuran-5-yl)-N-{1-[4-methyl-3-(1-methyl-1H-pyrazol-4-yl)phenyl]ethyl}pyrimidin-4-amine

773. PFKFB3 inhibitor for use in neuroprotection, wherein a PFKFB3 inhibitor is 3-(1-{[6-(1,3-benzothiazol-6-yl)-2-methylpyrimidin-4-yl]amino}ethyl)-N-(2-hydroxyethyl)benzene-1-sulfonamide

774. PFKFB3 inhibitor for use in neuroprotection, wherein a PFKFB3 inhibitor is N-(1-{3-[3-(dimethylamino)propoxy]phenyl}ethyl)-2-methyl-6-(3-methyl-1-benzofuran-5-yl)pyrimidin-4-amine

775. PFKFB3 inhibitor for use in neuroprotection, wherein a PFKFB3 inhibitor is 6-(1,3-benzothiazol-6-yl)-2-methyl-N-{1-[3-(morpholin-4-yl)phenyl]ethyl}pyrimidin-4-amine

776. PFKFB3 inhibitor for use in neuroprotection, wherein a PFKFB3 inhibitor is 6-(1,3-benzothiazol-6-yl)-N-(3,4-dihydro-2H-1-benzothiopyran-4-yl)-2-methylpyrimidin-4-amine

777. PFKFB3 inhibitor for use in neuroprotection, wherein a PFKFB3 inhibitor is N-(1-{3-[(1,3-dimethyl-1H-pyrazol-5-yl)methoxy]phenyl}ethyl)-2-methyl-6-(1-methyl-1H-indol-2-yl)pyrimidin-4-amine

778. PFKFB3 inhibitor for use in neuroprotection, wherein a PFKFB3 inhibitor is 1-(4-{2-[3-(1-{[6-(1,3-benzothiazol-6-yl)-2-methylpyrimidin-4-yl]amino}ethyl)phenoxy]ethyl}piperazin-1-yl)ethan-1-one

779. PFKFB3 inhibitor for use in neuroprotection, wherein a PFKFB3 inhibitor is 3-[(5-{3-[(1S)-1-{[2-methyl-6-(3-methyl-1-benzofuran-5-yl)pyrimidin-4-yl]amino}ethyl]phenyl}pyrimidin-2-yl)amino]propan-1-ol

780. PFKFB3 inhibitor for use in neuroprotection, wherein a PFKFB3 inhibitor is 6-(4-chloro-3-fluorophenyl)-2-methyl-N-[(1S)-1-{3-[6-(piperazin-1-yl)pyridin-3-yl]phenyl}ethyl]pyrimidin-4-amine

781. PFKFB3 inhibitor for use in neuroprotection, wherein a PFKFB3 inhibitor is 2-methyl-6-(3-methyl-1-benzofuran-5-yl)-N-[(1S)-1-{3-[6-(methylamino)pyridin-3-yl]phenyl}ethyl]pyrimidin-4-amine

782. PFKFB3 inhibitor for use in neuroprotection, wherein a PFKFB3 inhibitor is 6-(1,3-benzothiazol-6-yl)-2-methyl-N-(1-{3-[2-(1H-pyrrol-1-yl)ethoxy]phenyl}ethyl)pyrimidin-4-amine

783. PFKFB3 inhibitor for use in neuroprotection, wherein a PFKFB3 inhibitor is 2-methyl-6-(3-methylphenyl)-N-[(1R)-1,2,3,4-tetrahydronaphthalen-1-yl]pyrimidin-4-amine

784. PFKFB3 inhibitor for use in neuroprotection, wherein a PFKFB3 inhibitor is 1-(5-{5-[(1S)-1-{[2-methyl-6-(3-methyl-1-benzofuran-5-yl)pyrimidin-4-yl]amino}ethyl]pyridin-3-yl}pyrimidin-2-yl)piperidin-4-ol

785. PFKFB3 inhibitor for use in neuroprotection, wherein a PFKFB3 inhibitor is 2-[3-({[6-(1,3-benzothiazol-6-yl)-2-methylpyrimidin-4-yl]amino}methyl)-4-fluorophenoxy]-N,N-diethylacetamide

786. PFKFB3 inhibitor for use in neuroprotection, wherein a PFKFB3 inhibitor is N,N-dimethyl-2-{3-[(1S)-1-{[2-methyl-6-(3-methyl-1-benzofuran-5-yl)pyrimidin-4-yl]amino}ethyl]phenoxy}acetamide

787. PFKFB3 inhibitor for use in neuroprotection, wherein a PFKFB3 inhibitor is 2-methyl-6-(3-methyl-1-benzofuran-5-yl)-N-{1-[3-(1H-1,2,3,4-tetrazol-1-yl)phenyl]ethyl}pyrimidin-4-amine

788. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-1-(2-methylaziridin-1-yl)ethan-1-one

789. PFKFB3 inhibitor for use in neuroprotection, wherein a PFKFB3 inhibitor is [2-chloro-5-(2-methyl-6-{[(1R)-1,2,3,4-tetrahydronaphthalen-1-yl]amino}pyrimidin-4-yl)phenyl]methanol

790. PFKFB3 inhibitor for use in neuroprotection, wherein a PFKFB3 inhibitor is 6-(1,3-benzothiazol-6-yl)-N-(6-methoxy-1,2,3,4-tetrahydronaphthalen-1-yl)-2-methylpyrimidin-4-amine

791. PFKFB3 inhibitor for use in neuroprotection, wherein a PFKFB3 inhibitor is 2-methyl-6-(4-methylphenyl)-N-[(1R)-1,2,3,4-tetrahydronaphthalen-1-yl]pyrimidin-4-amine

792. PFKFB3 inhibitor for use in neuroprotection, wherein a PFKFB3 inhibitor is 6-(3-fluorophenyl)-2-methyl-N-[(1R)-1,2,3,4-tetrahydronaphthalen-1-yl]pyrimidin-4-amine

793. PFKFB3 inhibitor for use in neuroprotection, wherein a PFKFB3 inhibitor is 6-(1,3-benzothiazol-6-yl)-N-[(1S)-1-[3-(2-fluoropyridin-4-yl)phenyl]ethyl]-2-methylpyrimidin-4-amine

794. PFKFB3 inhibitor for use in neuroprotection, wherein a PFKFB3 inhibitor is 2-methyl-5-(2-methyl-6-{[(1R)-1,2,3,4-tetrahydronaphthalen-1-yl]amino}pyrimidin-4-yl)phenol

795. PFKFB3 inhibitor for use in neuroprotection, wherein a PFKFB3 inhibitor is 6-(1-benzothiophen-5-yl)-2-methyl-N-[(1R)-1,2,3,4-tetrahydronaphthalen-1-yl]pyrimidin-4-amine

796. PFKFB3 inhibitor for use in neuroprotection, wherein a PFKFB3 inhibitor is 6-(2,3-dihydro-1-benzofuran-5-yl)-2-methyl-N-{1-[3-(1-methyl-1H-pyrazol-4-yl)phenyl]ethyl}pyrimidin-4-amine

797. PFKFB3 inhibitor for use in neuroprotection, wherein a PFKFB3 inhibitor is 3-(1-{[2-methyl-6-(1-methyl-1H-indol-6-yl)pyrimidin-4-yl]amino}ethyl)benzene-1-sulfonamide

798. PFKFB3 inhibitor for use in neuroprotection, wherein a PFKFB3 inhibitor is N-[(1S)-1-{3-[6-(4-ethylpiperazin-1-yl)pyridin-3-yl]phenyl}ethyl]-2-methyl-6-(3-methyl-1-benzofuran-5-yl)pyrimidin-4-amine

799. PFKFB3 inhibitor for use in neuroprotection, wherein a PFKFB3 inhibitor is N,N-dimethyl-2-[3-(1-{[2-methyl-6-(3-methyl-1-benzofuran-5-yl)pyrimidin-4-yl]amino}ethyl)phenoxy]acetamide

800. PFKFB3 inhibitor for use in neuroprotection, wherein a PFKFB3 inhibitor is 2-methyl-6-(3-methyl-1-benzofuran-5-yl)-N-[(1S)-1-[3-(2H-1,2,3-triazol-2-yl)phenyl]ethyl]pyrimidin-4-amine

801. PFKFB3 inhibitor for use in neuroprotection, wherein a PFKFB3 inhibitor is 6-(1,3-benzothiazol-6-yl)-2-methyl-N-[1-(pyridin-3-yl)ethyl]pyrimidin-4-amine

802. PFKFB3 inhibitor for use in neuroprotection, wherein a PFKFB3 inhibitor is N-[(1S)-1-{3-[2-(ethylamino)pyrimidin-5-yl]phenyl}ethyl]-2-methyl-6-(3-methyl-1-benzofuran-5-yl)pyrimidin-4-amine

803. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N,N-dimethylacetamide

804. PFKFB3 inhibitor for use in neuroprotection, wherein a PFKFB3 inhibitor is methyl 2-chloro-5-(2-methyl-6-{[(1R)-1,2,3,4-tetrahydronaphthalen-1-yl]amino}pyrimidin-4-yl)benzoate

805. PFKFB3 inhibitor for use in neuroprotection, wherein a PFKFB3 inhibitor is N-(5-{[6-(1,3-benzothiazol-6-yl)-2-methylpyrimidin-4-yl]amino}-5,6,7,8-tetrahydronaphthalen-2-yl)propanamide

806. PFKFB3 inhibitor for use in neuroprotection, wherein a PFKFB3 inhibitor is 6-(4-chloro-3-fluorophenyl)-2-methyl-N-{1-[3-(1-methyl-1H-pyrazol-4-yl)phenyl]ethyl}pyrimidin-4-amine

807. PFKFB3 inhibitor for use in neuroprotection, wherein a PFKFB3 inhibitor is 2-[3-(1-{[6-(3-methoxy-4-methylphenyl)-2-methylpyrimidin-4-yl]amino}ethyl)phenoxy]acetamide

808. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-(2-hydroxyethyl)acetamide

809. PFKFB3 inhibitor for use in neuroprotection, wherein a PFKFB3 inhibitor is 2-{[2-methyl-5-(2-methyl-6-{[(1R)-1,2,3,4-tetrahydronaphthalen-1-yl]amino}pyrimidin-4-yl)phenyl]amino}acetonitrile

810. PFKFB3 inhibitor for use in neuroprotection, wherein a PFKFB3 inhibitor is N-[(1S)-1-[3-(6-chloropyridin-3-yl)phenyl]ethyl]-2-methyl-6-(3-methyl-1-benzofuran-5-yl)pyrimidin-4-amine

811. PFKFB3 inhibitor for use in neuroprotection, wherein a PFKFB3 inhibitor is N-[3-(1-{[2-methyl-6-(3-methyl-1-benzofuran-5-yl)pyrimidin-4-yl]amino}ethyl)phenyl]prop-2-enamide

812. PFKFB3 inhibitor for use in neuroprotection, wherein a PFKFB3 inhibitor is N,N-dimethyl-2-[3-(1-{[2-methyl-6-(1-methyl-1H-indol-6-yl)pyrimidin-4-yl]amino}ethyl)phenoxy]acetamide

813. PFKFB3 inhibitor for use in neuroprotection, wherein a PFKFB3 inhibitor is N-[(1S)-1-[3-(2-{4-[2-(dimethylamino)ethyl]piperazin-1-yl}pyrimidin-5-yl)phenyl]ethyl]-2-methyl-6-(3-methyl-1-benzothiophen-5-yl)pyrimidin-4-amine

814. PFKFB3 inhibitor for use in neuroprotection, wherein a PFKFB3 inhibitor is 4-[3-(1-{[6-(1,3-benzothiazol-6-yl)-2-methylpyrimidin-4-yl]amino}ethyl)phenoxy]butanoic acid

815. PFKFB3 inhibitor for use in neuroprotection, wherein a PFKFB3 inhibitor is 2-[3-(1-{[2-methyl-6-(1-methyl-1H-indol-6-yl)pyrimidin-4-yl]amino}ethyl)phenoxy]acetic acid

816. PFKFB3 inhibitor for use in neuroprotection, wherein a PFKFB3 inhibitor is 6-(1,3-benzothiazol-6-yl)-N-(1-{3-[6-(dimethylamino)pyridin-3-yl]phenyl}ethyl)-2-methylpyrimidin-4-amine

817. PFKFB3 inhibitor for use in neuroprotection, wherein a PFKFB3 inhibitor is 2-methyl-6-(3-methyl-1-benzofuran-5-yl)-N-[(1S)-1-{3-[6-(4-methylpiperazin-1-yl)pyridin-3-yl]phenyl}ethyl]pyrimidin-4-amine

818. PFKFB3 inhibitor for use in neuroprotection, wherein a PFKFB3 inhibitor is ethyl 2-{2-[3-(1-{[2-methyl-6-(1-methyl-1H-indol-6-yl)pyrimidin-4-yl]amino}ethyl)phenoxy]acetamido}acetate

819. PFKFB3 inhibitor for use in neuroprotection, wherein a PFKFB3 inhibitor is 6-(1,3-benzothiazol-6-yl)-2-methyl-N-[(1S)-1-[3-(1-methyl-1H-pyrazol-5-yl)phenyl]ethyl]pyrimidin-4-amine

820. PFKFB3 inhibitor for use in neuroprotection, wherein a PFKFB3 inhibitor is 6-(3-fluorophenyl)-2-methyl-N-[(1S)-1-[3-(1-methyl-1H-pyrazol-4-yl)phenyl]ethyl]pyrimidin-4-amine

821. PFKFB3 inhibitor for use in neuroprotection, wherein a PFKFB3 inhibitor is 2-methyl-N-{1-[3-(1-methyl-1H-pyrazol-4-yl)phenyl]ethyl}-6-(3-methylphenyl)pyrimidin-4-amine

822. PFKFB3 inhibitor for use in neuroprotection, wherein a PFKFB3 inhibitor is N-[(1S)-1-(3-methoxyphenyl)ethyl]-2-methyl-6-phenylpyrimidin-4-amine

823. PFKFB3 inhibitor for use in neuroprotection, wherein a PFKFB3 inhibitor is 2-methyl-6-(3-methyl-1-benzofuran-5-yl)-N-[(1S)-1-[3-(2-{4-[(1-methyl-1H-imidazol-2-yl)methyl]piperazin-1-yl}pyrimidin-5-yl)phenyl]ethyl]pyrimidin-4-amine

824. PFKFB3 inhibitor for use in neuroprotection, wherein a PFKFB3 inhibitor is N-{1-[5-(2-aminopyrimidin-5-yl)pyridin-3-yl]ethyl}-6-(4-chloro-3-fluorophenyl)-2-methylpyrimidin-4-amine

825. PFKFB3 inhibitor for use in neuroprotection, wherein a PFKFB3 inhibitor is 2-methyl-6-(3-methyl-1-benzofuran-5-yl)-N-[(1S)-1-[3-(1-methyl-1H-pyrazol-5-yl)phenyl]ethyl]pyrimidin-4-amine

826. PFKFB3 inhibitor for use in neuroprotection, wherein a PFKFB3 inhibitor is 6-(1,3-benzothiazol-6-yl)-2-methyl-N-{1-[3-(5-methyl-1H-1,2,3,4-tetrazol-1-yl)phenyl]ethyl}pyrimidin-4-amine

827. PFKFB3 inhibitor for use in neuroprotection, wherein a PFKFB3 inhibitor is 6-(1,3-benzothiazol-6-yl)-N-[(1S)-1-[3-(2-chloropyrimidin-5-yl)phenyl]ethyl]-2-methylpyrimidin-4-amine

828. PFKFB3 inhibitor for use in neuroprotection, wherein a PFKFB3 inhibitor is 2-methyl-1-[3-(1-{[2-methyl-6-(3-methyl-1-benzofuran-5-yl)pyrimidin-4-yl]amino}ethyl)phenoxy]propan-2-ol

829. PFKFB3 inhibitor for use in neuroprotection, wherein a PFKFB3 inhibitor is 2-methyl-6-(3-methyl-1-benzofuran-5-yl)-N-[(1R)-1-[3-(1H-1,2,3,4-tetrazol-1-yl)phenyl]ethyl]pyrimidin-4-amine

830. PFKFB3 inhibitor for use in neuroprotection, wherein a PFKFB3 inhibitor is 1-(5-{3-[(1S)-1-{[2-methyl-6-(3-methyl-1-benzofuran-5-yl)pyrimidin-4-yl]amino}ethyl]phenyl}pyridin-3-yl)ethan-1-one

831. PFKFB3 inhibitor for use in neuroprotection, wherein a PFKFB3 inhibitor is 6-(1,3-benzothiazol-6-yl)-2-methyl-N-(1-{3-[(piperidin-4-yl)methoxy]phenyl}ethyl)pyrimidin-4-amine

832. PFKFB3 inhibitor for use in neuroprotection, wherein a PFKFB3 inhibitor is 6-(7-fluoro-3-methyl-1-benzofuran-5-yl)-2-methyl-N-{1-[5-(1-methyl-1H-pyrazol-4-yl)pyridin-3-yl]ethyl}pyrimidin-4-amine

833. PFKFB3 inhibitor for use in neuroprotection, wherein a PFKFB3 inhibitor is N-[(1S)-1-[3-(6-aminopyridin-3-yl)phenyl]ethyl]-2-methyl-6-(3-methyl-1-benzofuran-5-yl)pyrimidin-4-amine

834. PFKFB3 inhibitor for use in neuroprotection, wherein a PFKFB3 inhibitor is 6-(3-methoxy-4-methylphenyl)-N-[(1S)-1-(3-methoxyphenyl)ethyl]-2-methylpyrimidin-4-amine

835. PFKFB3 inhibitor for use in neuroprotection, wherein a PFKFB3 inhibitor is 2-[3-({[6-(1,3-benzothiazol-6-yl)-2-methylpyrimidin-4-yl]amino}methyl)-4-fluorophenoxy]-N,N-dimethylacetamide

836. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-1-(morpholin-4-yl)ethan-1-one

837. PFKFB3 inhibitor for use in neuroprotection, wherein a PFKFB3 inhibitor is 6-(1,3-benzothiazol-6-yl)-2-methyl-N-[(2R)-2-phenylpropyl]pyrimidin-4-amine

838. PFKFB3 inhibitor for use in neuroprotection, wherein a PFKFB3 inhibitor is 3-(2-methyl-6-{[(1R)-1,2,3,4-tetrahydronaphthalen-1-yl]amino}pyrimidin-4-yl)phenol

839. PFKFB3 inhibitor for use in neuroprotection, wherein a PFKFB3 inhibitor is 2-(4-{3-[(1S)-1-{[2-methyl-6-(3-methyl-1-benzofuran-5-yl)pyrimidin-4-yl]amino}ethyl]phenyl}-1H-pyrazol-1-yl)acetic acid

840. PFKFB3 inhibitor for use in neuroprotection, wherein a PFKFB3 inhibitor is 6-(1,3-benzothiazol-6-yl)-N-[(1S)-1-{[1,1′-biphenyl]-3-yl}ethyl]-2-methylpyrimidin-4-amine

841. PFKFB3 inhibitor for use in neuroprotection, wherein a PFKFB3 inhibitor is 6-(3-methoxy-4-methylphenyl)-2-methyl-N-(1-{3-[(1-methyl-1H-pyrazol-3-yl)methoxy]phenyl}ethyl)pyrimidin-4-amine

842. PFKFB3 inhibitor for use in neuroprotection, wherein a PFKFB3 inhibitor is 6-(1,3-benzothiazol-6-yl)-N-(2,2-dimethyl-3,4-dihydro-2H-1-benzopyran-4-yl)-2-methylpyrimidin-4-amine

843. PFKFB3 inhibitor for use in neuroprotection, wherein a PFKFB3 inhibitor is 6-(1,3-benzothiazol-6-yl)-N-(1-{5-[(1,3-dimethyl-1H-pyrazol-5-yl)methoxy]pyridin-3-yl}ethyl)-2-methylpyrimidin-4-amine

844. PFKFB3 inhibitor for use in neuroprotection, wherein a PFKFB3 inhibitor is 6-(1,3-benzothiazol-6-yl)-N-[(1S)-1-(3-bromophenyl)ethyl]-2-methylpyrimidin-4-amine

845. PFKFB3 inhibitor for use in neuroprotection, wherein a PFKFB3 inhibitor is 2-methyl-6-(3-methyl-1-benzofuran-5-yl)-N-[(1S)-1-(3-{4-methyl-2H,3H,4H-pyrido[3,2-b][1,4]oxazin-7-yl}phenyl)ethyl]pyrimidin-4-amine

846. PFKFB3 inhibitor for use in neuroprotection, wherein a PFKFB3 inhibitor is 6-(3,4-difluorophenyl)-2-methyl-N-{1-[3-(1-methyl-1H-pyrazol-4-yl)phenyl]ethyl}pyrimidin-4-amine

847. PFKFB3 inhibitor for use in neuroprotection, wherein a PFKFB3 inhibitor is 4-[3-(1-{[6-(1,3-benzothiazol-6-yl)-2-methylpyrimidin-4-yl]amino}ethyl)phenoxy]butan-1-ol

848. PFKFB3 inhibitor for use in neuroprotection, wherein a PFKFB3 inhibitor is 6-(1,3-benzothiazol-6-yl)-N-[(1S)-1-{3-[2-(diethylamino)pyrimidin-5-yl]phenyl}ethyl]-2-methylpyrimidin-4-amine

849. PFKFB3 inhibitor for use in neuroprotection, wherein a PFKFB3 inhibitor is 6-(1,3-benzothiazol-6-yl)-2-methyl-N-[(1S)-1-[3-(6-methylpyridin-3-yl)phenyl]ethyl]pyrimidin-4-amine

850. PFKFB3 inhibitor for use in neuroprotection, wherein a PFKFB3 inhibitor is 6-(1,3-benzothiazol-6-yl)-N-[1-(3-bromophenyl)ethyl]-2-methylpyrimidin-4-amine

851. PFKFB3 inhibitor for use in neuroprotection, wherein a PFKFB3 inhibitor is 6-(1,3-benzothiazol-6-yl)-N-[(1S)-1-[3-(6-fluoro-5-methylpyridin-3-yl)phenyl]ethyl]-2-methylpyrimidin-4-amine

852. PFKFB3 inhibitor for use in neuroprotection, wherein a PFKFB3 inhibitor is 2-[3-(1-{[2-methyl-6-(3-methyl-1-benzofuran-5-yl)pyrimidin-4-yl]amino}ethyl)phenoxy]acetamide

853. PFKFB3 inhibitor for use in neuroprotection, wherein a PFKFB3 inhibitor is 6-(2-chloro-3-methoxyphenyl)-2-methyl-N-[(1R)-1,2,3,4-tetrahydronaphthalen-1-yl]pyrimidin-4-amine

854. PFKFB3 inhibitor for use in neuroprotection, wherein a PFKFB3 inhibitor is N,N-dimethyl-2-{3-[(1R)-1-{[2-methyl-6-(3-methyl-1-benzofuran-5-yl)pyrimidin-4-yl]amino}ethyl]phenoxy}acetamide

855. PFKFB3 inhibitor for use in neuroprotection, wherein a PFKFB3 inhibitor is N-[3-(1-{[6-(1,3-benzothiazol-6-yl)-2-methylpyrimidin-4-yl]amino}ethyl)phenyl]-1-methyl-1H-pyrazole-3-carboxamide

856. PFKFB3 inhibitor for use in neuroprotection, wherein a PFKFB3 inhibitor is 6-(1,3-benzothiazol-6-yl)-2-methyl-N-(1-{3-[(1H-pyrazol-5-yl)methoxy]phenyl}ethyl)pyrimidin-4-amine

857. PFKFB3 inhibitor for use in neuroprotection, wherein a PFKFB3 inhibitor is N-[(1S)-1-[3-(2-aminopyrimidin-5-yl)phenyl]ethyl]-6-(3-ethyl-1-benzofuran-5-yl)-2-methylpyrimidin-4-amine

858. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-cyclopropylacetamide

859. PFKFB3 inhibitor for use in neuroprotection, wherein a PFKFB3 inhibitor is 2-methyl-6-(1-methyl-1H-indol-5-yl)-N-[(1R)-1,2,3,4-tetrahydronaphthalen-1-yl]pyrimidin-4-amine

860. PFKFB3 inhibitor for use in neuroprotection, wherein a PFKFB3 inhibitor is 1-(5-{3-[(1S)-1-{[6-(4-chloro-3-fluorophenyl)-2-methylpyrimidin-4-yl]amino}ethyl]phenyl}pyrimidin-2-yl)piperidin-4-ol

861. PFKFB3 inhibitor for use in neuroprotection, wherein a PFKFB3 inhibitor is 6-(1,3-benzothiazol-6-yl)-2-methyl-N-(1-{3-[6-(piperazin-1-yl)pyridin-3-yl]phenyl}ethyl)pyrimidin-4-amine

862. PFKFB3 inhibitor for use in neuroprotection, wherein a PFKFB3 inhibitor is 6-(1,3-benzothiazol-6-yl)-2-methyl-N-(1-{3-[3-(pyrrolidin-1-yl)propoxy]phenyl}ethyl)pyrimidin-4-amine

863. PFKFB3 inhibitor for use in neuroprotection, wherein a PFKFB3 inhibitor is 6-(1-benzothiophen-5-yl)-N-[(1S)-1-(3-methoxyphenyl)ethyl]-2-methylpyrimidin-4-amine

864. PFKFB3 inhibitor for use in neuroprotection, wherein a PFKFB3 inhibitor is 6-(4-chloro-3-methylphenyl)-2-methyl-N-{1-[3-(1-methyl-1H-pyrazol-4-yl)phenyl]ethyl}pyrimidin-4-amine

865. PFKFB3 inhibitor for use in neuroprotection, wherein a PFKFB3 inhibitor is N-[(1S)-1-[3-(2-aminopyrimidin-5-yl)phenyl]ethyl]-2-methyl-6-(3-methyl-1-benzothiophen-5-yl)pyrimidin-4-amine

866. PFKFB3 inhibitor for use in neuroprotection, wherein a PFKFB3 inhibitor is 2-methyl-6-(3-methyl-1-benzofuran-5-yl)-N-[(1S)-1-[3-(pyridin-3-yl)phenyl]ethyl]pyrimidin-4-amine

867. PFKFB3 inhibitor for use in neuroprotection, wherein a PFKFB3 inhibitor is 6-(1,3-benzothiazol-6-yl)-2-methyl-N-{[1-(1-methylcyclopropanecarbonyl)piperidin-3-yl]methyl}pyrimidin-4-amine

868. PFKFB3 inhibitor for use in neuroprotection, wherein a PFKFB3 inhibitor is 6-(1,3-benzothiazol-6-yl)-2-methyl-N-(2-methyl-1,2,3,4-tetrahydronaphthalen-1-yl)pyrimidin-4-amine

869. PFKFB3 inhibitor for use in neuroprotection, wherein a PFKFB3 inhibitor is N-[(1S)-1-(3-methoxyphenyl)ethyl]-2-methyl-6-(4-methylphenyl)pyrimidin-4-amine

870. PFKFB3 inhibitor for use in neuroprotection, wherein a PFKFB3 inhibitor is 5′-(1-{[2-methyl-6-(3-methyl-1-benzofuran-5-yl)pyrimidin-4-yl]amino}ethyl)-[3,3′-bipyridin]-5-amine

871. PFKFB3 inhibitor for use in neuroprotection, wherein a PFKFB3 inhibitor is 6-(1,3-benzothiazol-6-yl)-N-[(1S)-1-[3-(6-chloropyridin-3-yl)phenyl]ethyl]-2-methylpyrimidin-4-amine

872. PFKFB3 inhibitor for use in neuroprotection, wherein a PFKFB3 inhibitor is 2-[3-(1-{[2-methyl-6-(1-methyl-1H-indol-6-yl)pyrimidin-4-yl]amino}ethyl)phenoxy]-1-(morpholin-4-yl)ethan-1-one

873. PFKFB3 inhibitor for use in neuroprotection, wherein a PFKFB3 inhibitor is 2-methyl-N-{1-[3-(1-methyl-1H-pyrazol-4-yl)phenyl]ethyl}-6-[4-(propan-2-yl)phenyl]pyrimidin-4-amine

874. PFKFB3 inhibitor for use in neuroprotection, wherein a PFKFB3 inhibitor is 1-[3-(1-{[2-methyl-6-(3-methyl-1-benzofuran-5-yl)pyrimidin-4-yl]amino}ethyl)phenoxy]propan-2-one

875. PFKFB3 inhibitor for use in neuroprotection, wherein a PFKFB3 inhibitor is 6-(3,4-dihydro-2H-1,4-benzoxazin-6-yl)-2-methyl-N-[(1R)-1,2,3,4-tetrahydronaphthalen-1-yl]pyrimidin-4-amine

876. PFKFB3 inhibitor for use in neuroprotection, wherein a PFKFB3 inhibitor is N-{1-[2-(2-aminopyrimidin-5-yl)pyridin-4-yl]ethyl}-2-methyl-6-(3-methyl-1-benzofuran-5-yl)pyrimidin-4-amine

877. PFKFB3 inhibitor for use in neuroprotection, wherein a PFKFB3 inhibitor is N-[(1S)-1-[3-(2-aminopyrimidin-5-yl)phenyl]ethyl]-6-(7-fluoro-1-benzofuran-5-yl)-2-methylpyrimidin-4-amine

878. PFKFB3 inhibitor for use in neuroprotection, wherein a PFKFB3 inhibitor is N-[(1S)-1-[3-(2-{[(furan-3-yl)methyl]amino}pyrimidin-5-yl)phenyl]ethyl]-2-methyl-6-(3-methyl-1-benzofuran-5-yl)pyrimidin-4-amine

879. PFKFB3 inhibitor for use in neuroprotection, wherein a PFKFB3 inhibitor is N-[1-(3-bromo-5-fluorophenyl)ethyl]-2-methyl-6-(3-methyl-1-benzofuran-5-yl)pyrimidin-4-amine

880. PFKFB3 inhibitor for use in neuroprotection, wherein a PFKFB3 inhibitor is 1-(5-{3-[(1S)-1-{[6-(7-fluoro-3-methyl-1-benzofuran-5-yl)-2-methylpyrimidin-4-yl]amino}ethyl]phenyl}pyrimidin-2-yl)piperidin-4-ol

881. PFKFB3 inhibitor for use in neuroprotection, wherein a PFKFB3 inhibitor is 5-{3-[(1S)-1-{[2-methyl-6-(3-methyl-1-benzofuran-5-yl)pyrimidin-4-yl]amino}ethyl]phenyl}-1,2-dihydropyrimidin-2-one

882. PFKFB3 inhibitor for use in neuroprotection, wherein a PFKFB3 inhibitor is 4-[3-(1-{[2-methyl-6-(1-methyl-1H-indol-6-yl)pyrimidin-4-yl]amino}ethyl)phenoxy]butanoic acid

883. PFKFB3 inhibitor for use in neuroprotection, wherein a PFKFB3 inhibitor is 6-(1,3-benzothiazol-6-yl)-2-methyl-N-(1-{3-[3-(morpholin-4-yl)propoxy]phenyl}ethyl)pyrimidin-4-amine

884. PFKFB3 inhibitor for use in neuroprotection, wherein a PFKFB3 inhibitor is N,N-diethyl-2-[3-(1-{[2-methyl-6-(naphthalen-2-yl)pyrimidin-4-yl]amino}ethyl)phenoxy]acetamide

885. PFKFB3 inhibitor for use in neuroprotection, wherein a PFKFB3 inhibitor is N-[1-(3-bromo-4-methylphenyl)ethyl]-2-methyl-6-(3-methyl-1-benzofuran-5-yl)pyrimidin-4-amine

886. PFKFB3 inhibitor for use in neuroprotection, wherein a PFKFB3 inhibitor is 6-(1,3-benzothiazol-6-yl)-N-(1-{3-[3-(dimethylamino)propoxy]phenyl}ethyl)-2-methylpyrimidin-4-amine

887. PFKFB3 inhibitor for use in neuroprotection, wherein a PFKFB3 inhibitor is 6-(4-chlorophenyl)-2-methyl-N-[(1R)-1,2,3,4-tetrahydronaphthalen-1-yl]pyrimidin-4-amine

888. PFKFB3 inhibitor for use in neuroprotection, wherein a PFKFB3 inhibitor is 6-(1,3-benzothiazol-6-yl)-N-{1-[3-(2,6-dimethylpyridin-3-yl)phenyl]ethyl}-2-methylpyrimidin-4-amine

889. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-2-methylpropanamide

890. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-(propan-2-yl)acetamide

891. PFKFB3 inhibitor for use in neuroprotection, wherein a PFKFB3 inhibitor is 2-methyl-6-(3-methyl-1-benzofuran-5-yl)-N-[(1S)-1-[3-(1H-pyrazol-1-yl)phenyl]ethyl]pyrimidin-4-amine

892. PFKFB3 inhibitor for use in neuroprotection, wherein a PFKFB3 inhibitor is 6-(1,3-benzothiazol-6-yl)-2-methyl-N-[(1S)-1-[3-(pyrimidin-5-yl)phenyl]ethyl]pyrimidin-4-amine

893. PFKFB3 inhibitor for use in neuroprotection, wherein a PFKFB3 inhibitor is 2-methyl-6-(3-methyl-1-benzofuran-5-yl)-N-{1-[5-(1-methyl-1H-pyrazol-4-yl)pyridin-3-yl]ethyl}pyrimidin-4-amine

894. PFKFB3 inhibitor for use in neuroprotection, wherein a PFKFB3 inhibitor is N-[(1S)-1-[4-fluoro-3-(1-methyl-1H-pyrazol-4-yl)phenyl]ethyl]-2-methyl-6-(3-methyl-1-benzofuran-5-yl)pyrimidin-4-amine

895. PFKFB3 inhibitor for use in neuroprotection, wherein a PFKFB3 inhibitor is 1-[3-(1-{[6-(1,3-benzothiazol-6-yl)-2-methylpyrimidin-4-yl]amino}ethyl)phenoxy]propan-2-one

896. PFKFB3 inhibitor for use in neuroprotection, wherein a PFKFB3 inhibitor is 6-(1,3-benzothiazol-6-yl)-2-methyl-N-(1-{3-[2-(2-methyl-1H-imidazol-1-yl)ethoxy]phenyl}ethyl)pyrimidin-4-amine

897. PFKFB3 inhibitor for use in neuroprotection, wherein a PFKFB3 inhibitor is N-[(1S)-1-[3-(2-aminopyrimidin-5-yl)phenyl]ethyl]-2-methyl-6-(3-methyl-1-benzofuran-5-yl)pyrimidin-4-amine

898. PFKFB3 inhibitor for use in neuroprotection, wherein a PFKFB3 inhibitor is N-[(1S)-1-[3-(2-aminopyrimidin-5-yl)phenyl]ethyl]-6-(4-chloro-3-fluorophenyl)-2-methylpyrimidin-4-amine

899. PFKFB3 inhibitor for use in neuroprotection, wherein a PFKFB3 inhibitor is 2,2-dimethyl-3-[(5-{3-[(1S)-1-{[2-methyl-6-(3-methyl-1-benzofuran-5-yl)pyrimidin-4-yl]amino}ethyl]phenyl}pyrimidin-2-yl)amino]propan-1-ol

900. PFKFB3 inhibitor for use in neuroprotection, wherein a PFKFB3 inhibitor is 6-(1,3-benzothiazol-6-yl)-2-methyl-N-(1-{3-[2-(1H-pyrazol-1-yl)ethoxy]phenyl}ethyl)pyrimidin-4-amine

901. PFKFB3 inhibitor for use in neuroprotection, wherein a PFKFB3 inhibitor is N-(1-{3-[(1,3-dimethyl-1H-pyrazol-5-yl)methoxy]phenyl}ethyl)-6-(3-ethyl-1-benzofuran-5-yl)-2-methylpyrimidin-4-amine

902. PFKFB3 inhibitor for use in neuroprotection, wherein a PFKFB3 inhibitor is N-[(1S)-1-[3-(2-aminopyrimidin-5-yl)phenyl]ethyl]-6-(4-chloro-3,5-difluorophenyl)-2-methylpyrimidin-4-amine

903. PFKFB3 inhibitor for use in neuroprotection, wherein a PFKFB3 inhibitor is N-[(1S)-1-{3-[2-(4-ethylpiperazin-1-yl)pyrimidin-5-yl]phenyl}ethyl]-2-methyl-6-(3-methyl-1-benzofuran-5-yl)pyrimidin-4-amine

904. PFKFB3 inhibitor for use in neuroprotection, wherein a PFKFB3 inhibitor is N,N-diethyl-2-[3-(1-{[2-methyl-6-(3-methyl-1-benzofuran-5-yl)pyrimidin-4-yl]amino}ethyl)phenoxy]acetamide

905. PFKFB3 inhibitor for use in neuroprotection, wherein a PFKFB3 inhibitor is N-[(1R)-1-[4-fluoro-3-(1-methyl-1H-pyrazol-4-yl)phenyl]ethyl]-2-methyl-6-(3-methyl-1-benzofuran-5-yl)pyrimidin-4-amine

906. PFKFB3 inhibitor for use in neuroprotection, wherein a PFKFB3 inhibitor is 6-(7-fluoro-3-methyl-1-benzofuran-5-yl)-N-[(1S)-1-[3-(6-fluoropyridin-3-yl)phenyl]ethyl]-2-methylpyrimidin-4-amine

907. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-1-(piperidin-1-yl)ethan-1-one

908. PFKFB3 inhibitor for use in neuroprotection, wherein a PFKFB3 inhibitor is 6-(1-benzofuran-5-yl)-2-methyl-N-(1-{3-[(1-methyl-1H-pyrazol-3-yl)methoxy]phenyl}ethyl)pyrimidin-4-amine

909. PFKFB3 inhibitor for use in neuroprotection, wherein a PFKFB3 inhibitor is 6-(2-chloro-3-methoxyphenyl)-N-(2,3-dihydro-1H-inden-1-yl)-2-methylpyrimidin-4-amine

910. PFKFB3 inhibitor for use in neuroprotection, wherein a PFKFB3 inhibitor is 3-(1-{[6-(3-methoxy-4-methylphenyl)-2-methylpyrimidin-4-yl]amino}ethyl)phenol

911. PFKFB3 inhibitor for use in neuroprotection, wherein a PFKFB3 inhibitor is N-[(1S)-1-{[1,1′-biphenyl]-3-yl}ethyl]-2-methyl-6-(3-methyl-1-benzofuran-5-yl)pyrimidin-4-amine

912. PFKFB3 inhibitor for use in neuroprotection, wherein a PFKFB3 inhibitor is 6-(2-chloro-3-methoxyphenyl)-N-[(1S)-1-(3-methoxyphenyl)ethyl]-2-methylpyrimidin-4-amine

913. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N-(2-hydroxypropyl)acetamide

914. PFKFB3 inhibitor for use in neuroprotection, wherein a PFKFB3 inhibitor is 6-(1,3-benzothiazol-6-yl)-N-[(1S)-1-[3-(5-fluoropyridin-3-yl)phenyl]ethyl]-2-methylpyrimidin-4-amine

915. PFKFB3 inhibitor for use in neuroprotection, wherein a PFKFB3 inhibitor is 3-[(1S)-1-{[6-(1,3-benzothiazol-6-yl)-2-methylpyrimidin-4-yl]amino}ethyl]phenol

916. PFKFB3 inhibitor for use in neuroprotection, wherein a PFKFB3 inhibitor is 3-(1-{[6-(1,3-benzothiazol-6-yl)-2-methylpyrimidin-4-yl]amino}ethyl)benzene-1-carboximidamide

917. PFKFB3 inhibitor for use in neuroprotection, wherein a PFKFB3 inhibitor is N-[(1S)-1-[3-(2-{4-[2-(dimethylamino)ethyl]piperazin-1-yl}pyrimidin-5-yl)phenyl]ethyl]-2-methyl-6-(3-methyl-1-benzofuran-5-yl)pyrimidin-4-amine

918. PFKFB3 inhibitor for use in neuroprotection, wherein a PFKFB3 inhibitor is 2-methyl-6-(3-methyl-1-benzofuran-5-yl)-N-[(1S)-1-[3-(pyrimidin-5-yl)phenyl]ethyl]pyrimidin-4-amine

919. PFKFB3 inhibitor for use in neuroprotection, wherein a PFKFB3 inhibitor is N-[(1S)-1-[5-(2-aminopyrimidin-5-yl)pyridin-3-yl]ethyl]-2-methyl-6-(3-methyl-1-benzofuran-5-yl)pyrimidin-4-amine

920. PFKFB3 inhibitor for use in neuroprotection, wherein a PFKFB3 inhibitor is 6-(4-chloro-3-methylphenyl)-2-methyl-N-[(1R)-1,2,3,4-tetrahydronaphthalen-1-yl]pyrimidin-4-amine

921. PFKFB3 inhibitor for use in neuroprotection, wherein a PFKFB3 inhibitor is 2-methyl-6-[4-methyl-3-(prop-2-yn-1-yloxy)phenyl]-N-[(1R)-1,2,3,4-tetrahydronaphthalen-1-yl]pyrimidin-4-amine

922. PFKFB3 inhibitor for use in neuroprotection, wherein a PFKFB3 inhibitor is 6-(1,3-benzothiazol-6-yl)-2-methyl-N-{1-[3-(4H-1,2,4-triazol-4-yl)phenyl]ethyl}pyrimidin-4-amine

923. PFKFB3 inhibitor for use in neuroprotection, wherein a PFKFB3 inhibitor is 6-(1,3-benzothiazol-6-yl)-N-[1-(2-methoxypyridin-4-yl)ethyl]-2-methylpyrimidin-4-amine

924. PFKFB3 inhibitor for use in neuroprotection, wherein a PFKFB3 inhibitor is N-[(1S)-1-(4-fluorophenyl)ethyl]-2-methyl-6-(1-methyl-1H-indol-6-yl)pyrimidin-4-amine

925. PFKFB3 inhibitor for use in neuroprotection, wherein a PFKFB3 inhibitor is N-[(1S)-1-[3-(5-fluoropyridin-3-yl)phenyl]ethyl]-2-methyl-6-(3-methyl-1-benzofuran-5-yl)pyrimidin-4-amine

926. PFKFB3 inhibitor for use in neuroprotection, wherein a PFKFB3 inhibitor is N-[(1S)-1-{3-[6-(dimethylamino)pyridin-3-yl]phenyl}ethyl]-2-methyl-6-(3-methyl-1-benzofuran-5-yl)pyrimidin-4-amine

927. PFKFB3 inhibitor for use in neuroprotection, wherein a PFKFB3 inhibitor is 6-(1,3-benzothiazol-6-yl)-N-[2-(cyclohex-1-en-1-yl)ethyl]-2-methylpyrimidin-4-amine

928. PFKFB3 inhibitor for use in neuroprotection, wherein a PFKFB3 inhibitor is 1-[3-(1-{[6-(1,3-benzothiazol-6-yl)-2-methylpyrimidin-4-yl]amino}ethyl)phenoxy]-3-fluoropropan-2-ol

929. PFKFB3 inhibitor for use in neuroprotection, wherein a PFKFB3 inhibitor is N-[(1S)-1-[3-(2-aminopyrimidin-5-yl)phenyl]ethyl]-6-(3-chloro-1-benzofuran-5-yl)-2-methylpyrimidin-4-amine

930. PFKFB3 inhibitor for use in neuroprotection, wherein a PFKFB3 inhibitor is N-[(4R)-3,4-dihydro-2H-1-benzopyran-4-yl]-2-methyl-6-(3-methyl-1-benzofuran-5-yl)pyrimidin-4-amine

931. PFKFB3 inhibitor for use in neuroprotection, wherein a PFKFB3 inhibitor is 6-(3-fluorophenyl)-N-[(1S)-1-(3-methoxyphenyl)ethyl]-2-methylpyrimidin-4-amine

932. PFKFB3 inhibitor for use in neuroprotection, wherein a PFKFB3 inhibitor is 6-(1,3-benzothiazol-6-yl)-2-methyl-N-{1-[3-(1-methyl-1H-pyrazol-4-yl)phenyl]ethyl}pyrimidin-4-amine

933. PFKFB3 inhibitor for use in neuroprotection, wherein a PFKFB3 inhibitor is 2-{[2-fluoro-5-(2-methyl-6-{[(1R)-1,2,3,4-tetrahydronaphthalen-1-yl]amino}pyrimidin-4-yl)phenyl]amino}acetonitrile

934. PFKFB3 inhibitor for use in neuroprotection, wherein a PFKFB3 inhibitor is 6-(2,5-dimethylphenyl)-2-methyl-N-{1-[3-(1-methyl-1H-pyrazol-4-yl)phenyl]ethyl}pyrimidin-4-amine

935. PFKFB3 inhibitor for use in neuroprotection, wherein a PFKFB3 inhibitor is 6-(1,3-benzothiazol-6-yl)-N-(1-{4-fluoro-3-[(1-methyl-1H-pyrazol-5-yl)methoxy]phenyl}ethyl)-2-methylpyrimidin-4-amine

936. PFKFB3 inhibitor for use in neuroprotection, wherein a PFKFB3 inhibitor is 6-(1,3-benzothiazol-6-yl)-N-(1-{3-[3-(diethylamino)propoxy]phenyl}ethyl)-2-methylpyrimidin-4-amine

937. PFKFB3 inhibitor for use in neuroprotection, wherein a PFKFB3 inhibitor is 6-(1,3-benzothiazol-6-yl)-2-methyl-N-[(1S)-1,2,3,4-tetrahydronaphthalen-1-yl]pyrimidin-4-amine

938. PFKFB3 inhibitor for use in neuroprotection, wherein a PFKFB3 inhibitor is 6-(1,3-benzothiazol-6-yl)-2-methyl-N-[1-(3-propoxyphenyl)ethyl]pyrimidin-4-amine

939. PFKFB3 inhibitor for use in neuroprotection, wherein a PFKFB3 inhibitor is 6-(4-chloro-3-fluorophenyl)-N-[(1S)-1-[3-(1-ethyl-1H-pyrazol-4-yl)phenyl]ethyl]-2-methylpyrimidin-4-amine

940. PFKFB3 inhibitor for use in neuroprotection, wherein a PFKFB3 inhibitor is 2-fluoro-4-[2-methyl-6-({1-[3-(1-methyl-1H-pyrazol-4-yl)phenyl]ethyl}amino)pyrimidin-4-yl]benzamide

941. PFKFB3 inhibitor for use in neuroprotection, wherein a PFKFB3 inhibitor is 2-[3-(1-{[6-(1,3-benzothiazol-6-yl)-2-methylpyrimidin-4-yl]amino}ethyl)phenoxy]-N,N-diethylacetamide

942. PFKFB3 inhibitor for use in neuroprotection, wherein a PFKFB3 inhibitor is 2-methyl-6-(3-methyl-1-benzofuran-5-yl)-N-[(1S)-1-[5-(1H-pyrazol-4-yl)pyridin-3-yl]ethyl]pyrimidin-4-amine

943. PFKFB3 inhibitor for use in neuroprotection, wherein a PFKFB3 inhibitor is N-[(1S)-1-(4-fluorophenyl)ethyl]-2-methyl-6-(3-methyl-1-benzofuran-5-yl)pyrimidin-4-amine

944. PFKFB3 inhibitor for use in neuroprotection, wherein a PFKFB3 inhibitor is 6-(furan-3-yl)-2-methyl-N-[(1R)-1,2,3,4-tetrahydronaphthalen-1-yl]pyrimidin-4-amine

945. PFKFB3 inhibitor for use in neuroprotection, wherein a PFKFB3 inhibitor is 1-(azepan-1-yl)-2-[3-(1-{[6-(1,3-benzothiazol-6-yl)-2-methylpyrimidin-4-yl]amino}ethyl)phenoxy]ethan-1-one

946. PFKFB3 inhibitor for use in neuroprotection, wherein a PFKFB3 inhibitor is 6-(1,3-benzothiazol-6-yl)-N-{1-[3-(2-fluoropyridin-3-yl)phenyl]ethyl}-2-methylpyrimidin-4-amine

947. PFKFB3 inhibitor for use in neuroprotection, wherein a PFKFB3 inhibitor is N-[(4R)-3,4-dihydro-2H-1-benzopyran-4-yl]-2-methyl-6-(1-methyl-1H-indol-6-yl)pyrimidin-4-amine

948. PFKFB3 inhibitor for use in neuroprotection, wherein a PFKFB3 inhibitor is N-[1-(3-bromophenyl)ethyl]-6-(2-chloro-3-methoxyphenyl)-2-methylpyrimidin-4-amine

949. PFKFB3 inhibitor for use in neuroprotection, wherein a PFKFB3 inhibitor is 6-(1,3-benzothiazol-6-yl)-N-{1-[3-(5-methoxypyridin-3-yl)phenyl]ethyl}-2-methylpyrimidin-4-amine

950. PFKFB3 inhibitor for use in neuroprotection, wherein a PFKFB3 inhibitor is 2-methyl-6-(3-methyl-1-benzofuran-5-yl)-N-[(1S)-1-[3-(2-{4-[2-(morpholin-4-yl)ethyl]piperazin-1-yl}pyrimidin-5-yl)phenyl]ethyl]pyrimidin-4-amine

951. PFKFB3 inhibitor for use in neuroprotection, wherein a PFKFB3 inhibitor is 6-(2,3-dihydro-1,4-benzodioxin-6-yl)-2-methyl-N-[(1R)-1,2,3,4-tetrahydronaphthalen-1-yl]pyrimidin-4-amine

952. PFKFB3 inhibitor for use in neuroprotection, wherein a PFKFB3 inhibitor is 6-(3-ethylphenyl)-N-[(1S)-1-(3-methoxyphenyl)ethyl]-2-methylpyrimidin-4-amine

953. PFKFB3 inhibitor for use in neuroprotection, wherein a PFKFB3 inhibitor is 6-(1,3-benzothiazol-6-yl)-N-[(1S)-1-{3-[(1,3-dimethyl-1H-pyrazol-5-yl)methoxy]phenyl}ethyl]-2-methylpyrimidin-4-amine

954. PFKFB3 inhibitor for use in neuroprotection, wherein a PFKFB3 inhibitor is 6-(4-chlorophenyl)-N-[(1S)-1-(3-methoxyphenyl)ethyl]-2-methylpyrimidin-4-amine

955. PFKFB3 inhibitor for use in neuroprotection, wherein a PFKFB3 inhibitor is 2-methyl-6-(1-methyl-1H-indol-6-yl)-N-{1-[3-(1-methyl-1H-pyrazol-4-yl)phenyl]ethyl}pyrimidin-4-amine

956. PFKFB3 inhibitor for use in neuroprotection, wherein a PFKFB3 inhibitor is 2-iodoacetic acid

957. PFKFB3 inhibitor for use in neuroprotection, wherein a PFKFB3 inhibitor is 2,3,4-trichloro-N-[3-(2H-1,2,3,4-tetrazol-5-yl)phenyl]benzene-1-sulfonamide

958. PFKFB3 inhibitor for use in neuroprotection, wherein a PFKFB3 inhibitor is 5-(1,3-oxazol-5-yl)-N-[3-(2H-1,2,3,4-tetrazol-5-yl)phenyl]thiophene-2-sulfonamide

959. PFKFB3 inhibitor for use in neuroprotection, wherein a PFKFB3 inhibitor is 5-chloro-3-methyl-N-[3-(1,3-oxazol-5-yl)phenyl]-1-benzothiophene-2-sulfonamide

960. PFKFB3 inhibitor for use in neuroprotection, wherein a PFKFB3 inhibitor is 3-methyl-5-(propan-2-yl)-N-[3-(2H-1,2,3,4-tetrazol-5-yl)phenyl]-1-benzothiophene-2-sulfonamide

961. PFKFB3 inhibitor for use in neuroprotection, wherein a PFKFB3 inhibitor is 3-[3-methyl-5-(propan-2-yl)-1-benzothiophene-2-sulfonamido]benzoic acid

962. PFKFB3 inhibitor for use in neuroprotection, wherein a PFKFB3 inhibitor is 2-[3-methyl-5-(propan-2-yl)-1-benzothiophene-2-sulfonamido]pyridine-4-carboxylic acid

963. PFKFB3 inhibitor for use in neuroprotection, wherein a PFKFB3 inhibitor is 2-[3-methyl-5-(propan-2-yl)-1-benzothiophene-2-sulfonamido]-1,3-thiazole-5-carboxylic acid

964. PFKFB3 inhibitor for use in neuroprotection, wherein a PFKFB3 inhibitor is 4-methyl-2-[3-methyl-5-(propan-2-yl)-1-benzothiophene-2-sulfonamido]-1,3-thiazole-5-carboxylic acid

965. PFKFB3 inhibitor for use in neuroprotection, wherein a PFKFB3 inhibitor is 5-[3-methyl-5-(propan-2-yl)-1-benzothiophene-2-sulfonamido]pyridine-3-carboxylic acid

966. PFKFB3 inhibitor for use in neuroprotection, wherein a PFKFB3 inhibitor is 6-[3-methyl-5-(propan-2-yl)-1-benzothiophene-2-sulfonamido]pyridine-2-carboxylic acid

967. PFKFB3 inhibitor for use in neuroprotection, wherein a PFKFB3 inhibitor is 3-[3-methyl-5-(propan-2-yl)-1-benzothiophene-2-sulfonamido]-5-nitrobenzoic acid

968. PFKFB3 inhibitor for use in neuroprotection, wherein a PFKFB3 inhibitor is 2-(5-{3-[3-methyl-5-(propan-2-yl)-1-benzothiophene-2-sulfonamido]phenyl}-2H-1,2,3,4-tetrazol-2-yl)acetic acid

969. PFKFB3 inhibitor for use in neuroprotection, wherein a PFKFB3 inhibitor is 3-methyl-5-(propan-2-yl)-N-[3-(2H-1,2,3,4-tetrazol-5-yl)phenyl]-1-benzofuran-2-sulfonamide 970. PFKFB3 inhibitor for use in neuroprotection, wherein a PFKFB3 inhibitor is 3-[3-methyl-5-(propan-2-yl)-1-benzofuran-2-sulfonamido]benzoic acid

971. PFKFB3 inhibitor for use in neuroprotection, wherein a PFKFB3 inhibitor is 2-{3-[3-methyl-5-(propan-2-yl)-1-benzothiophene-2-sulfonamido]phenyl}acetic acid

972. PFKFB3 inhibitor for use in neuroprotection, wherein a PFKFB3 inhibitor is N-[3-(2H-1,2,3,4-tetrazol-5-yl)phenyl]-[1,1′-biphenyl]-3-sulfonamide

973. PFKFB3 inhibitor for use in neuroprotection, wherein a PFKFB3 inhibitor is 4′-fluoro-N-[3-(2H-1,2,3,4-tetrazol-5-yl)phenyl]-[1,1′-biphenyl]-3-sulfonamide

974. PFKFB3 inhibitor for use in neuroprotection, wherein a PFKFB3 inhibitor is 2,6-dichloro-N-[3-(2H-1,2,3,4-tetrazol-5-yl)phenyl]-4-(trifluoromethyl)benzene-1-sulfonamide

975. PFKFB3 inhibitor for use in neuroprotection, wherein a PFKFB3 inhibitor is 4′-methoxy-N-[3-(2H-1,2,3,4-tetrazol-5-yl)phenyl]-[1,1′-biphenyl]-3-sulfonamide

976. PFKFB3 inhibitor for use in neuroprotection, wherein a PFKFB3 inhibitor is N-[3-(2H-1,2,3,4-tetrazol-5-yl)phenyl]naphthalene-2-sulfonamide

977. PFKFB3 inhibitor for use in neuroprotection, wherein a PFKFB3 inhibitor is 5-[2-(methylsulfanyl)pyrimidin-4-yl]-N-[3-(2H-1,2,3,4-tetrazol-5-yl)phenyl]thiophene-2-sulfonamide

978. PFKFB3 inhibitor for use in neuroprotection, wherein a PFKFB3 inhibitor is 5-(5-fluoro-2-methoxyphenyl)-N-[3-(2H-1,2,3,4-tetrazol-5-yl)phenyl]thiophene-2-sulfonamide

979. PFKFB3 inhibitor for use in neuroprotection, wherein a PFKFB3 inhibitor is 5-(3,5-difluorophenyl)-N-[3-(2H-1,2,3,4-tetrazol-5-yl)phenyl]thiophene-2-sulfonamide

980. PFKFB3 inhibitor for use in neuroprotection, wherein a PFKFB3 inhibitor is 5-(2-methoxyphenyl)-N-[3-(2H-1,2,3,4-tetrazol-5-yl)phenyl]thiophene-2-sulfonamide

981. PFKFB3 inhibitor for use in neuroprotection, wherein a PFKFB3 inhibitor is 5-(2-methyl-1,3-thiazol-4-yl)-N-[3-(2H-1,2,3,4-tetrazol-5-yl)phenyl]thiophene-2-sulfonamide

982. PFKFB3 inhibitor for use in neuroprotection, wherein a PFKFB3 inhibitor is 5-(2-chlorophenyl)-N-[3-(2H-1,2,3,4-tetrazol-5-yl)phenyl]thiophene-2-sulfonamide

983. PFKFB3 inhibitor for use in neuroprotection, wherein a PFKFB3 inhibitor is 5-(4-methylphenyl)-N-[3-(2H-1,2,3,4-tetrazol-5-yl)phenyl]thiophene-2-sulfonamide

984. PFKFB3 inhibitor for use in neuroprotection, wherein a PFKFB3 inhibitor is 5-(2,4-dimethoxyphenyl)-N-[3-(2H-1,2,3,4-tetrazol-5-yl)phenyl]thiophene-2-sulfonamide

985. PFKFB3 inhibitor for use in neuroprotection, wherein a PFKFB3 inhibitor is 5-(4-chlorophenyl)-N-[3-(2H-1,2,3,4-tetrazol-5-yl)phenyl]thiophene-2-sulfonamide

986. PFKFB3 inhibitor for use in neuroprotection, wherein a PFKFB3 inhibitor is 5-(4-fluoro-2-methoxyphenyl)-N-[3-(2H-1,2,3,4-tetrazol-5-yl)phenyl]thiophene-2-sulfonamide

987. PFKFB3 inhibitor for use in neuroprotection, wherein a PFKFB3 inhibitor is N-[3-(2H-1,2,3,4-tetrazol-5-yl)phenyl]-[1,1′-biphenyl]-4-sulfonamide

988. PFKFB3 inhibitor for use in neuroprotection, wherein a PFKFB3 inhibitor is 5-chloro-3-methyl-N-[3-(2H-1,2,3,4-tetrazol-5-yl)phenyl]-1-benzothiophene-2-sulfonamide

989. PFKFB3 inhibitor for use in neuroprotection, wherein a PFKFB3 inhibitor is 3,5-dimethyl-N-[3-(2H-1,2,3,4-tetrazol-5-yl)phenyl]-1-benzothiophene-2-sulfonamide

990. PFKFB3 inhibitor for use in neuroprotection, wherein a PFKFB3 inhibitor is 5-bromo-3-methyl-N-[3-(2H-1,2,3,4-tetrazol-5-yl)phenyl]-1-benzothiophene-2-sulfonamide

991. PFKFB3 inhibitor for use in neuroprotection, wherein a PFKFB3 inhibitor is 7-methoxy-3-methyl-N-[3-(2H-1,2,3,4-tetrazol-5-yl)phenyl]-1-benzothiophene-2-sulfonamide

992. PFKFB3 inhibitor for use in neuroprotection, wherein a PFKFB3 inhibitor is 7-chloro-3-methyl-N-[3-(2H-1,2,3,4-tetrazol-5-yl)phenyl]-1-benzothiophene-2-sulfonamide

993. PFKFB3 inhibitor for use in neuroprotection, wherein a PFKFB3 inhibitor is 5-methoxy-3-methyl-N-[3-(2H-1,2,3,4-tetrazol-5-yl)phenyl]-1-benzothiophene-2-sulfonamide

994. PFKFB3 inhibitor for use in neuroprotection, wherein a PFKFB3 inhibitor is 3-(5-chloro-3-methyl-1-benzothiophene-2-sulfonamido)benzoic acid

995. PFKFB3 inhibitor for use in neuroprotection, wherein a PFKFB3 inhibitor is 3-methyl-N-[3-(2H-1,2,3,4-tetrazol-5-yl)phenyl]-1-benzothiophene-2-sulfonamide

996. PFKFB3 inhibitor for use in neuroprotection, wherein a PFKFB3 inhibitor is 5-methyl-N-[3-(2H-1,2,3,4-tetrazol-5-yl)phenyl]-1-benzothiophene-2-sulfonamide

997. PFKFB3 inhibitor for use in neuroprotection, wherein a PFKFB3 inhibitor is 3-(5-bromo-3-methyl-1-benzothiophene-2-sulfonamido)benzoic acid

998. PFKFB3 inhibitor for use in neuroprotection, wherein a PFKFB3 inhibitor is 3-(7-methoxy-3-methyl-1l-benzothiophene-2-sulfonamido)benzoic acid

999. PFKFB3 inhibitor for use in neuroprotection, wherein a PFKFB3 inhibitor is 5-fluoro-3-methyl-N-[3-(2H-1,2,3,4-tetrazol-5-yl)phenyl]-1-benzothiophene-2-sulfonamide

1000. PFKFB3 inhibitor for use in neuroprotection, wherein a PFKFB3 inhibitor is 3-(7-chloro-3-methyl-1-benzothiophene-2-sulfonamido)benzoic acid

1001. PFKFB3 inhibitor for use in neuroprotection, wherein a PFKFB3 inhibitor is 3-methyl-5-(pyrrolidin-1-yl)-N-[3-(2H-1,2,3,4-tetrazol-5-yl)phenyl]-1-benzothiophene-2-sulfonamide

1002. PFKFB3 inhibitor for use in neuroprotection, wherein a PFKFB3 inhibitor is 3-[3-methyl-5-(pyrrolidin-1-yl)-1-benzothiophene-2-sulfonamido]benzoic acid

1003. PFKFB3 inhibitor for use in neuroprotection, wherein a PFKFB3 inhibitor is 3-(5-amino-3-methyl-1-benzothiophene-2-sulfonamido)benzoic acid

1004. PFKFB3 inhibitor for use in neuroprotection, wherein a PFKFB3 inhibitor is 5-amino-3-methyl-N-[3-(2H-1,2,3,4-tetrazol-5-yl)phenyl]-1-benzothiophene-2-sulfonamide

1005. PFKFB3 inhibitor for use in neuroprotection, wherein a PFKFB3 inhibitor is 3-(5-acetamido-3-methyl-1-benzothiophene-2-sulfonamido)benzoic acid

1006. PFKFB3 inhibitor for use in neuroprotection, wherein a PFKFB3 inhibitor is 4′-methyl-N-[3-(2H-1,2,3,4-tetrazol-5-yl)phenyl]-[1,1′-biphenyl]-3-sulfonamide

1007. PFKFB3 inhibitor for use in neuroprotection, wherein a PFKFB3 inhibitor is 2,4-dichloro-5-methyl-N-[3-(2H-1,2,3,4-tetrazol-5-yl)phenyl]benzene-1-sulfonamide

1008. PFKFB3 inhibitor for use in neuroprotection, wherein a PFKFB3 inhibitor is 3′,5′-dichloro-N-[3-(2H-1,2,3,4-tetrazol-5-yl)phenyl]-[1,1′-biphenyl]-3-sulfonamide

1009. PFKFB3 inhibitor for use in neuroprotection, wherein a PFKFB3 inhibitor is 2,4-dichloro-6-methyl-N-[3-(2H-1,2,3,4-tetrazol-5-yl)phenyl]benzene-1-sulfonamide

1010. PFKFB3 inhibitor for use in neuroprotection, wherein a PFKFB3 inhibitor is 3-{3′,5′-dichloro-[1,1′-biphenyl]-3-sulfonamido}benzoic acid

1011. PFKFB3 inhibitor for use in neuroprotection, wherein a PFKFB3 inhibitor is N-[3-(2H-1,2,3,4-tetrazol-5-yl)phenyl]-4′-(trifluoromethyl)-[1,1′-biphenyl]-3-sulfonamide

1012. PFKFB3 inhibitor for use in neuroprotection, wherein a PFKFB3 inhibitor is 4-bromo-N-[3-(2H-1,2,3,4-tetrazol-5-yl)phenyl]-2-(trifluoromethyl)benzene-1-sulfonamide

1013. PFKFB3 inhibitor for use in neuroprotection, wherein a PFKFB3 inhibitor is 4-bromo-2-fluoro-N-[3-(2H-1,2,3,4-tetrazol-5-yl)phenyl]benzene-1-sulfonamide

1014. PFKFB3 inhibitor for use in neuroprotection, wherein a PFKFB3 inhibitor is 3-(5-{[3-(2H-1,2,3,4-tetrazol-5-yl)phenyl]sulfamoyl}thiophen-2-yl)benzamide

1015. PFKFB3 inhibitor for use in neuroprotection, wherein a PFKFB3 inhibitor is 5-(5-chloro-1,2,4-thiadiazol-3-yl)-N-[3-(2H-1,2,3,4-tetrazol-5-yl)phenyl]thiophene-2-sulfonamide

1016. PFKFB3 inhibitor for use in neuroprotection, wherein a PFKFB3 inhibitor is 5-phenyl-N-[3-(2H-1,2,3,4-tetrazol-5-yl)phenyl]thiophene-2-sulfonamide

1017. PFKFB3 inhibitor for use in neuroprotection, wherein a PFKFB3 inhibitor is 3-{5-[2-(methylsulfanyl)pyrimidin-4-yl]thiophene-2-sulfonamido}benzoic acid

1018. PFKFB3 inhibitor for use in neuroprotection, wherein a PFKFB3 inhibitor is 3-[5-(2-methyl-1,3-thiazol-4-yl)thiophene-2-sulfonamido]benzoic acid

1019. PFKFB3 inhibitor for use in neuroprotection, wherein a PFKFB3 inhibitor is N-[3-(2H-1,2,3,4-tetrazol-5-yl)phenyl]-5-[5-(trifluoromethyl)-1,2-oxazol-3-yl]thiophene-2-sulfonamide

1020. PFKFB3 inhibitor for use in neuroprotection, wherein a PFKFB3 inhibitor is N-[3-(2H-1,2,3,4-tetrazol-5-yl)phenyl]-1-benzofuran-2-sulfonamide

1021. PFKFB3 inhibitor for use in neuroprotection, wherein a PFKFB3 inhibitor is 5-(1,2-oxazol-3-yl)-N-[3-(2H-1,2,3,4-tetrazol-5-yl)phenyl]thiophene-2-sulfonamide

1022. PFKFB3 inhibitor for use in neuroprotection, wherein a PFKFB3 inhibitor is 2,4,6-trichloro-N-[3-(2H-1,2,3,4-tetrazol-5-yl)phenyl]benzene-1-sulfonamide

1023. PFKFB3 inhibitor for use in neuroprotection, wherein a PFKFB3 inhibitor is 2,3-dichloro-N-[3-(2H-1,2,3,4-tetrazol-5-yl)phenyl]benzene-1-sulfonamide

1024. PFKFB3 inhibitor for use in neuroprotection, wherein a PFKFB3 inhibitor is 2,5-dichloro-N-[3-(2H-1,2,3,4-tetrazol-5-yl)phenyl]benzene-1-sulfonamide

1025. PFKFB3 inhibitor for use in neuroprotection, wherein a PFKFB3 inhibitor is 3-chloro-2-methyl-N-[3-(2H-1,2,3,4-tetrazol-5-yl)phenyl]benzene-1-sulfonamide

1026. PFKFB3 inhibitor for use in neuroprotection, wherein a PFKFB3 inhibitor is 2,4-dichloro-N-[3-(2H-1,2,3,4-tetrazol-5-yl)phenyl]benzene-1-sulfonamide

1027. PFKFB3 inhibitor for use in neuroprotection, wherein a PFKFB3 inhibitor is 2,4,5-trichloro-N-[3-(2H-1,2,3,4-tetrazol-5-yl)phenyl]benzene-1-sulfonamide

1028. PFKFB3 inhibitor for use in neuroprotection, wherein a PFKFB3 inhibitor is 2,4-difluoro-N-[3-(2H-1,2,3,4-tetrazol-5-yl)phenyl]benzene-1-sulfonamide

1029. PFKFB3 inhibitor for use in neuroprotection, wherein a PFKFB3 inhibitor is 7-chloro-N-[3-(2H-1,2,3,4-tetrazol-5-yl)phenyl]-2,1,3-benzoxadiazole-4-sulfonamide

1030. PFKFB3 inhibitor for use in neuroprotection, wherein a PFKFB3 inhibitor is methyl 3-(5-chloro-3-methyl-1-benzothiophene-2-sulfonamido)benzoate

1031. PFKFB3 inhibitor for use in neuroprotection, wherein a PFKFB3 inhibitor is 3-(3-methyl-1-benzothiophene-2-sulfonamido)benzoic acid

1032. PFKFB3 inhibitor for use in neuroprotection, wherein a PFKFB3 inhibitor is 3-(5-fluoro-3-methyl-1-benzothiophene-2-sulfonamido)benzoic acid

1033. PFKFB3 inhibitor for use in neuroprotection, wherein a PFKFB3 inhibitor is 3-(5-methoxy-3-methyl-1-benzothiophene-2-sulfonamido)benzoic acid

1034. PFKFB3 inhibitor for use in neuroprotection, wherein a PFKFB3 inhibitor is 3-[5-(pyridin-2-yl)thiophene-2-sulfonamido]benzoic acid

1035. PFKFB3 inhibitor for use in neuroprotection, wherein a PFKFB3 inhibitor is 3-[5-(1,2-oxazol-3-yl)thiophene-2-sulfonamido]benzoic acid

1036. PFKFB3 inhibitor for use in neuroprotection, wherein a PFKFB3 inhibitor is 3-{[1,1′-biphenyl]-3-sulfonamido}benzoic acid

1037. PFKFB3 inhibitor for use in neuroprotection, wherein a PFKFB3 inhibitor is 2-chloro-4-fluoro-N-[3-(2H-1,2,3,4-tetrazol-5-yl)phenyl]benzene-1-sulfonamide

1038. PFKFB3 inhibitor for use in neuroprotection, wherein a PFKFB3 inhibitor is N-[3-(2H-1,2,3,4-tetrazol-5-yl)phenyl]-1-benzothiophene-2-sulfonamide

1039. PFKFB3 inhibitor for use in neuroprotection, wherein a PFKFB3 inhibitor is 4-(1,3-oxazol-5-yl)-N-[3-(2H-1,2,3,4-tetrazol-5-yl)phenyl]benzene-1-sulfonamide

1040. PFKFB3 inhibitor for use in neuroprotection, wherein a PFKFB3 inhibitor is 3-(1-benzothiophene-2-sulfonamido)benzoic acid

1041. PFKFB3 inhibitor for use in neuroprotection, wherein a PFKFB3 inhibitor is 4′-methoxy-N-[3-(2H-1,2,3,4-tetrazol-5-yl)phenyl]-[1,1′-biphenyl]-4-sulfonamide

1042. PFKFB3 inhibitor for use in neuroprotection, wherein a PFKFB3 inhibitor is 3′,4′-dichloro-N-[3-(2H-1,2,3,4-tetrazol-5-yl)phenyl]-[1,1′-biphenyl]-4-sulfonamide

1043. PFKFB3 inhibitor for use in neuroprotection, wherein a PFKFB3 inhibitor is 5-(1,2-oxazol-5-yl)-N-[3-(2H-1,2,3,4-tetrazol-5-yl)phenyl]thiophene-2-sulfonamide

1044. PFKFB3 inhibitor for use in neuroprotection, wherein a PFKFB3 inhibitor is methyl 3-[5-(2-methyl-1,3-thiazol-4-yl)thiophene-2-sulfonamido]benzoate

1045. PFKFB3 inhibitor for use in neuroprotection, wherein a PFKFB3 inhibitor is 3-(5-methyl-1-benzothiophene-2-sulfonamido)benzoic acid

1046. PFKFB3 inhibitor for use in neuroprotection, wherein a PFKFB3 inhibitor is 4′-chloro-N-[3-(2H-1,2,3,4-tetrazol-5-yl)phenyl]-[1,1′-biphenyl]-3-sulfonamide

1047. PFKFB3 inhibitor for use in neuroprotection, wherein a PFKFB3 inhibitor is 3′,4′-dichloro-N-[3-(2H-1,2,3,4-tetrazol-5-yl)phenyl]-[1,1′-biphenyl]-3-sulfonamide

1048. PFKFB3 inhibitor for use in neuroprotection, wherein a PFKFB3 inhibitor is methyl 3-[3-methyl-5-(propan-2-yl)-1-benzothiophene-2-sulfonamido]benzoate

1049. PFKFB3 inhibitor for use in neuroprotection, wherein a PFKFB3 inhibitor is 3-{4′-chloro-[1,1′-biphenyl]-3-sulfonamido}benzoic acid

1050. PFKFB3 inhibitor for use in neuroprotection, wherein a PFKFB3 inhibitor is 3-{4′-fluoro-[1,1′-biphenyl]-3-sulfonamido}benzoic acid

1051. PFKFB3 inhibitor for use in neuroprotection, wherein a PFKFB3 inhibitor is 3-{4′-methoxy-[1,1′-biphenyl]-3-sulfonamido}benzoic acid

1052. PFKFB3 inhibitor for use in neuroprotection, wherein a PFKFB3 inhibitor is 3-{3′,4′-dichloro-[1,1′-biphenyl]-3-sulfonamido}benzoic acid

1053. PFKFB3 inhibitor for use in neuroprotection, wherein a PFKFB3 inhibitor is 5-(3-methoxyphenyl)-N-[3-(2H-1,2,3,4-tetrazol-5-yl)phenyl]thiophene-2-sulfonamide

1054. PFKFB3 inhibitor for use in neuroprotection, wherein a PFKFB3 inhibitor is 5-(3,4-dichlorophenyl)-N-[3-(2H-1,2,3,4-tetrazol-5-yl)phenyl]thiophene-2-sulfonamide

1055. PFKFB3 inhibitor for use in neuroprotection, wherein a PFKFB3 inhibitor is N-[3-(2H-1,2,3,4-tetrazol-5-yl)phenyl]-5-[3-(trifluoromethyl)phenyl]thiophene-2-sulfonamide

1056. PFKFB3 inhibitor for use in neuroprotection, wherein a PFKFB3 inhibitor is 5-(2-methylphenyl)-N-[3-(2H-1,2,3,4-tetrazol-5-yl)phenyl]thiophene-2-sulfonamide

1057. PFKFB3 inhibitor for use in neuroprotection, wherein a PFKFB3 inhibitor is 5-(2,4-difluorophenyl)-N-[3-(2H-1,2,3,4-tetrazol-5-yl)phenyl]thiophene-2-sulfonamide

1058. PFKFB3 inhibitor for use in neuroprotection, wherein a PFKFB3 inhibitor is 5-(3-chloro-4-fluorophenyl)-N-[3-(2H-1,2,3,4-tetrazol-5-yl)phenyl]thiophene-2-sulfonamide

1059. PFKFB3 inhibitor for use in neuroprotection, wherein a PFKFB3 inhibitor is 5-(3-chlorophenyl)-N-[3-(2H-1,2,3,4-tetrazol-5-yl)phenyl]thiophene-2-sulfonamide

1060. PFKFB3 inhibitor for use in neuroprotection, wherein a PFKFB3 inhibitor is 5-(pyridin-4-yl)-N-[3-(2H-1,2,3,4-tetrazol-5-yl)phenyl]thiophene-2-sulfonamide

1061. PFKFB3 inhibitor for use in neuroprotection, wherein a PFKFB3 inhibitor is 3-[3-methyl-5-(morpholin-4-yl)-1-benzothiophene-2-sulfonamido]benzoic acid

1062. PFKFB3 inhibitor for use in neuroprotection, wherein a PFKFB3 inhibitor is 2-(1H-pyrrol-1-yl)ethyl 3-(5-chloro-3-methyl-1-benzothiophene-2-sulfonamido)benzoate

1063. PFKFB3 inhibitor for use in neuroprotection, wherein a PFKFB3 inhibitor is ethyl 3-(5-chloro-3-methyl-1-benzothiophene-2-sulfonamido)benzoate

1064. PFKFB3 inhibitor for use in neuroprotection, wherein a PFKFB3 inhibitor is propan-2-yl 3-(5-chloro-3-methyl-1-benzothiophene-2-sulfonamido)benzoate

1065. PFKFB3 inhibitor for use in neuroprotection, wherein a PFKFB3 inhibitor is 2-methoxyethyl 3-(5-chloro-3-methyl-1-benzothiophene-2-sulfonamido)benzoate

1066. PFKFB3 inhibitor for use in neuroprotection, wherein a PFKFB3 inhibitor is butyl 3-(5-chloro-3-methyl-1-benzothiophene-2-sulfonamido)benzoate

1067. PFKFB3 inhibitor for use in neuroprotection, wherein a PFKFB3 inhibitor is benzyl 3-(5-chloro-3-methyl-1-benzothiophene-2-sulfonamido)benzoate

1068. PFKFB3 inhibitor for use in neuroprotection wherein a PFKFB3 inhibitor is propyl 3-(5-chloro-3-methyl-1-benzothiophene-2-sulfonamido)benzoate

1069. PFKFB3 inhibitor for use in neuroprotection wherein a PFKFB3 inhibitor is pentyl 3-(5-chloro-3-methyl-1-benzothiophene-2-sulfonamido)benzoate

1070. PFKFB3 inhibitor for use in neuroprotection, wherein a PFKFB3 inhibitor is hexyl 3-(5-chloro-3-methyl-1-benzothiophene-2-sulfonamido)benzoate

1071. PFKFB3 inhibitor for use in neuroprotection, wherein a PFKFB3 inhibitor is phenyl 3-(5-chloro-3-methyl-1-benzothiophene-2-sulfonamido)benzoate

1072. PFKFB3 inhibitor for use in neuroprotection, wherein a PFKFB3 inhibitor is oxolan-3-yl 3-(5-chloro-3-methyl-1-benzothiophene-2-sulfonamido)benzoate

1073. PFKFB3 inhibitor for use in neuroprotection, wherein a PFKFB3 inhibitor is (oxolan-3-yl)methyl 3-(5-chloro-3-methyl-1-benzothiophene-2-sulfonamido)benzoate

1074. PFKFB3 inhibitor for use in neuroprotection, wherein a PFKFB3 inhibitor is 3-(dimethylamino)propyl 3-(5-chloro-3-methyl-1-benzothiophene-2-sulfonamido)benzoate

1075. PFKFB3 inhibitor for use in neuroprotection, wherein a PFKFB3 inhibitor is methyl 2-(5-{3-[3-methyl-5-(propan-2-yl)-1-benzothiophene-2-sulfonamido]phenyl}-2H-1,2,3,4-tetrazol-2-yl)acetate

1076. PFKFB3 inhibitor for use in neuroprotection, wherein a PFKFB3 inhibitor is methyl 3-(5-bromo-3-methyl-1-benzothiophene-2-sulfonamido)benzoate

1077. PFKFB3 inhibitor for use in neuroprotection, wherein a PFKFB3 inhibitor is methyl 3-(7-methoxy-3-methyl-1-benzothiophene-2-sulfonamido)benzoate

1078. PFKFB3 inhibitor for use in neuroprotection, wherein a PFKFB3 inhibitor is methyl 3-(7-chloro-3-methyl-1-benzothiophene-2-sulfonamido)benzoate

1079. PFKFB3 inhibitor for use in neuroprotection, wherein a PFKFB3 inhibitor is methyl 3-[3-methyl-5-(propan-2-yl)-1-benzofuran-2-sulfonamido]benzoate

1080. PFKFB3 inhibitor for use in neuroprotection, wherein a PFKFB3 inhibitor is methyl 2-[3-methyl-5-(propan-2-yl)-1-benzothiophene-2-sulfonamido]-1,3-thiazole-5-carboxylate

1081. PFKFB3 inhibitor for use in neuroprotection, wherein a PFKFB3 inhibitor is ethyl 4-methyl-2-[3-methyl-5-(propan-2-yl)-1-benzothiophene-2-sulfonamido]-1,3-thiazole-5-carboxylate

1082. PFKFB3 inhibitor for use in neuroprotection, wherein a PFKFB3 inhibitor is ethyl 2-(5-chloro-3-methyl-1-benzothiophene-2-sulfonamido)-4-methyl-1,3-thiazole-5-carboxylate

1083. PFKFB3 inhibitor for use in neuroprotection, wherein a PFKFB3 inhibitor is ethyl 2-[5-chloro-4-(2,5-difluorophenyl)thiophene-2-sulfonamido]-4-methyl-1,3-thiazole-5-carboxylate

1084. PFKFB3 inhibitor for use in neuroprotection, wherein a PFKFB3 inhibitor is 2-(5-chloro-3-methyl-1-benzothiophene-2-sulfonamido)-4-methyl-1,3-thiazole-5-carboxylic acid

1085. PFKFB3 inhibitor for use in neuroprotection, wherein a PFKFB3 inhibitor is 2-[5-chloro-4-(2,5-difluorophenyl)thiophene-2-sulfonamido]-4-methyl-1,3-thiazole-5-carboxylic acid

1086. PFKFB3 inhibitor for use in neuroprotection, wherein a PFKFB3 inhibitor is 2-(5-chloro-3-methyl-1-benzothiophene-2-sulfonamido)-1,3-thiazole-5-carboxylic acid

1087. PFKFB3 inhibitor for use in neuroprotection, wherein a PFKFB3 inhibitor is 2-[5-(3,5-difluorophenyl)thiophene-2-sulfonamido]-5-methyl-1,3-thiazole-4-carboxylic acid

1088. PFKFB3 inhibitor for use in neuroprotection, wherein a PFKFB3 inhibitor is 2-[5-chloro-4-(2,5-difluorophenyl)thiophene-2-sulfonamido]-5-methyl-1,3-thiazole-4-carboxylic acid

1089. PFKFB3 inhibitor for use in neuroprotection, wherein a PFKFB3 inhibitor is 5-methyl-2-[3-methyl-5-(propan-2-yl)-1-benzothiophene-2-sulfonamido]-1,3-thiazole-4-carboxylic acid

1090. PFKFB3 inhibitor for use in neuroprotection, wherein a PFKFB3 inhibitor is 3-(5-chloro-3-methyl-1-benzothiophene-2-sulfonamido)-2-hydroxybenzoic acid

1091. PFKFB3 inhibitor for use in neuroprotection, wherein a PFKFB3 inhibitor is 3-[5-chloro-4-(2,5-difluorophenyl)thiophene-2-sulfonamido]-2-hydroxybenzoic acid

1092. PFKFB3 inhibitor for use in neuroprotection, wherein a PFKFB3 inhibitor is 3-[5-chloro-4-(2,3-dihydro-1-benzofuran-5-yl)thiophene-2-sulfonamido]-2-hydroxybenzoic acid

1093. PFKFB3 inhibitor for use in neuroprotection, wherein a PFKFB3 inhibitor is 3-[5-chloro-4-(2-hydroxyphenyl)thiophene-2-sulfonamido]-2-hydroxybenzoic acid

1094. PFKFB3 inhibitor for use in neuroprotection, wherein a PFKFB3 inhibitor is 5-(5-chloro-3-methyl-1-benzothiophene-2-sulfonamido)-2-hydroxybenzoic acid

1095. PFKFB3 inhibitor for use in neuroprotection, wherein a PFKFB3 inhibitor is 5-{4′-chloro-[1,1′-biphenyl]-3-sulfonamido}-2-hydroxybenzoic acid

1096. PFKFB3 inhibitor for use in neuroprotection, wherein a PFKFB3 inhibitor is methyl 5-(5-chloro-3-methyl-1-benzothiophene-2-sulfonamido)-2-hydroxybenzoate

1097. PFKFB3 inhibitor for use in neuroprotection, wherein a PFKFB3 inhibitor is 5-(benzenesulfonyl)-N-[3-(2H-1,2,3,4-tetrazol-5-yl)phenyl]thiophene-2-sulfonamide

1098. PFKFB3 inhibitor for use in neuroprotection, wherein a PFKFB3 inhibitor is 2,2-dimethyl-N-[3-(2H-1,2,3,4-tetrazol-5-yl)phenyl]-3,4-dihydro-2H-1-benzopyran-6-sulfonamide

1099. PFKFB3 inhibitor for use in neuroprotection, wherein a PFKFB3 inhibitor is 4-{[1,1′-biphenyl]-3-sulfonamido}-2-hydroxybenzoic acid

1100. PFKFB3 inhibitor for use in neuroprotection, wherein a PFKFB3 inhibitor is 4-(3-bromobenzenesulfonamido)-2-hydroxybenzoic acid

1101. PFKFB3 inhibitor for use in neuroprotection, wherein a PFKFB3 inhibitor is 4-[3-(5-acetylthiophen-2-yl)benzenesulfonamido]-2-hydroxybenzoic acid

1102. PFKFB3 inhibitor for use in neuroprotection, wherein a PFKFB3 inhibitor is 2-hydroxy-4-{4′-hydroxy-[1,1′-biphenyl]-3-sulfonamido}benzoic acid

1103. PFKFB3 inhibitor for use in neuroprotection, wherein a PFKFB3 inhibitor is 4-{3-[(E)-2-(4-fluorophenyl)ethenyl]benzenesulfonamido}-2-hydroxybenzoic acid

1104. PFKFB3 inhibitor for use in neuroprotection, wherein a PFKFB3 inhibitor is 4-{3′-amino-4′-methoxy-[1,1′-biphenyl]-3-sulfonamido}-2-hydroxybenzoic acid

1105. PFKFB3 inhibitor for use in neuroprotection, wherein a PFKFB3 inhibitor is 2-hydroxy-4-[3-(pyridin-3-yl)benzenesulfonamido]benzoic acid

1106. PFKFB3 inhibitor for use in neuroprotection, wherein a PFKFB3 inhibitor is 4-[4′-(dimethylamino)-[1,1′-biphenyl]-3-sulfonamido]-2-hydroxybenzoic acid

1107. PFKFB3 inhibitor for use in neuroprotection, wherein a PFKFB3 inhibitor is 2-hydroxy-4-[5-(trifluoromethyl)-[1,1′-biphenyl]-3-sulfonamido]benzoic acid

1108. PFKFB3 inhibitor for use in neuroprotection, wherein a PFKFB3 inhibitor is 4-{4,6-difluoro-[1,1′-biphenyl]-3-sulfonamido}-2-hydroxybenzoic acid

1109. PFKFB3 inhibitor for use in neuroprotection, wherein a PFKFB3 inhibitor is 2-hydroxy-4-{6-methoxy-[1,1′-biphenyl]-3-sulfonamido}benzoic acid

1110. PFKFB3 inhibitor for use in neuroprotection, wherein a PFKFB3 inhibitor is 4-(5-chloro-4-phenylthiophene-2-sulfonamido)-2-hydroxybenzoic acid

1111. PFKFB3 inhibitor for use in neuroprotection, wherein a PFKFB3 inhibitor is 2-hydroxy-4-[2′-(hydroxymethyl)-[1,1′-biphenyl]-3-sulfonamido]benzoic acid

1112. PFKFB3 inhibitor for use in neuroprotection, wherein a PFKFB3 inhibitor is 4-{3′-fluoro-[1,1′-biphenyl]-3-sulfonamido}-2-hydroxybenzoic acid

1113. PFKFB3 inhibitor for use in neuroprotection, wherein a PFKFB3 inhibitor is 4-{2′,6′-difluoro-[1,1′-biphenyl]-3-sulfonamido}-2-hydroxybenzoic acid

1114. PFKFB3 inhibitor for use in neuroprotection, wherein a PFKFB3 inhibitor is 2-hydroxy-4-{3′-[(propan-2-yloxy)carbonyl]-[1,1′-biphenyl]-3-sulfonamido}benzoic acid

1115. PFKFB3 inhibitor for use in neuroprotection, wherein a PFKFB3 inhibitor is 4-[3-(2,3-dihydro-1-benzofuran-5-yl)benzenesulfonamido]-2-hydroxybenzoic acid

1116. PFKFB3 inhibitor for use in neuroprotection, wherein a PFKFB3 inhibitor is 4-{3′-fluoro-4′-hydroxy-[1,1′-biphenyl]-3-sulfonamido}-2-hydroxybenzoic acid

1117. PFKFB3 inhibitor for use in neuroprotection, wherein a PFKFB3 inhibitor is 2-hydroxy-4-[3-(quinolin-6-yl)benzenesulfonamido]benzoic acid

1118. PFKFB3 inhibitor for use in neuroprotection, wherein a PFKFB3 inhibitor is 4-{3′-amino-[1,1′-biphenyl]-3-sulfonamido}-2-hydroxybenzoic acid

1119. PFKFB3 inhibitor for use in neuroprotection, wherein a PFKFB3 inhibitor is 2-hydroxy-4-[3-(2-methyl-1,3-thiazol-4-yl)benzenesulfonamido]benzoic acid

1120. PFKFB3 inhibitor for use in neuroprotection, wherein a PFKFB3 inhibitor is 4-(5-chlorothiophene-2-sulfonamido)-2-hydroxybenzoic acid

1121. PFKFB3 inhibitor for use in neuroprotection, wherein a PFKFB3 inhibitor is 4-[5-chloro-4-(2,3-dihydro-1-benzofuran-5-yl)thiophene-2-sulfonamido]-2-hydroxybenzoic acid

1122. PFKFB3 inhibitor for use in neuroprotection, wherein a PFKFB3 inhibitor is 4-[5-chloro-4-(3-fluoro-4-hydroxyphenyl)thiophene-2-sulfonamido]-2-hydroxybenzoic acid

1123. PFKFB3 inhibitor for use in neuroprotection, wherein a PFKFB3 inhibitor is 4-[5-chloro-4-(quinolin-6-yl)thiophene-2-sulfonamido]-2-hydroxybenzoic acid

1124. PFKFB3 inhibitor for use in neuroprotection, wherein a PFKFB3 inhibitor is 4-[4-(2H-1,3-benzodioxol-5-yl)-5-chlorothiophene-2-sulfonamido]-2-hydroxybenzoic acid

1125. PFKFB3 inhibitor for use in neuroprotection, wherein a PFKFB3 inhibitor is 4-[5-chloro-4-(4-hydroxy-3,5-dimethylphenyl)thiophene-2-sulfonamido]-2-hydroxybenzoic acid

1126. PFKFB3 inhibitor for use in neuroprotection, wherein a PFKFB3 inhibitor is 4-[5-chloro-4-(2,4-difluorophenyl)thiophene-2-sulfonamido]-2-hydroxybenzoic acid

1127. PFKFB3 inhibitor for use in neuroprotection, wherein a PFKFB3 inhibitor is 4-[4-(3-acetylphenyl)-5-chlorothiophene-2-sulfonamido]-2-hydroxybenzoic acid

1128. PFKFB3 inhibitor for use in neuroprotection, wherein a PFKFB3 inhibitor is 4-{5-chloro-4-[2-(hydroxymethyl)phenyl]thiophene-2-sulfonamido}-2-hydroxybenzoic acid

1129. PFKFB3 inhibitor for use in neuroprotection, wherein a PFKFB3 inhibitor is 4-[5-chloro-4-(3-fluorophenyl)thiophene-2-sulfonamido]-2-hydroxybenzoic acid

1130. PFKFB3 inhibitor for use in neuroprotection, wherein a PFKFB3 inhibitor is 4-(5-chloro-4-{3-[(propan-2-yloxy)carbonyl]phenyl}thiophene-2-sulfonamido)-2-hydroxybenzoic acid

1131. PFKFB3 inhibitor for use in neuroprotection, wherein a PFKFB3 inhibitor is 4-[5-chloro-4-(3,5-difluorophenyl)thiophene-2-sulfonamido]-2-hydroxybenzoic acid

1132. PFKFB3 inhibitor for use in neuroprotection, wherein a PFKFB3 inhibitor is 4-[5-chloro-4-(6-ethoxypyridin-3-yl)thiophene-2-sulfonamido]-2-hydroxybenzoic acid

1133. PFKFB3 inhibitor for use in neuroprotection, wherein a PFKFB3 inhibitor is 4-[4-(3-aminophenyl)-5-chlorothiophene-2-sulfonamido]-2-hydroxybenzoic acid

1134. PFKFB3 inhibitor for use in neuroprotection, wherein a PFKFB3 inhibitor is 4-[5-chloro-4-(4-methoxyphenyl)thiophene-2-sulfonamido]-2-hydroxybenzoic acid

1135. PFKFB3 inhibitor for use in neuroprotection, wherein a PFKFB3 inhibitor is 4-[4-(4-aminophenyl)-5-chlorothiophene-2-sulfonamido]-2-hydroxybenzoic acid

1136. PFKFB3 inhibitor for use in neuroprotection, wherein a PFKFB3 inhibitor is 4-[5-chloro-4-(4-hydroxyphenyl)thiophene-2-sulfonamido]-2-hydroxybenzoic acid

1137. PFKFB3 inhibitor for use in neuroprotection, wherein a PFKFB3 inhibitor is 4-{5-chloro-4-[3-(hydroxymethyl)phenyl]thiophene-2-sulfonamido}-2-hydroxybenzoic acid

1138. PFKFB3 inhibitor for use in neuroprotection, wherein a PFKFB3 inhibitor is 4-{5-chloro-4-[4-(hydroxymethyl)phenyl]thiophene-2-sulfonamido}-2-hydroxybenzoic acid

1139. PFKFB3 inhibitor for use in neuroprotection, wherein a PFKFB3 inhibitor is 4-[4-(3-amino-4-methoxyphenyl)-5-chlorothiophene-2-sulfonamido]-2-hydroxybenzoic acid

1140. PFKFB3 inhibitor for use in neuroprotection, wherein a PFKFB3 inhibitor is 4-[5-chloro-4-(4-methanesulfonamidophenyl)thiophene-2-sulfonamido]-2-hydroxybenzoic acid

1141. PFKFB3 inhibitor for use in neuroprotection, wherein a PFKFB3 inhibitor is 4-(7-chloro-3-methyl-1-benzothiophene-2-sulfonamido)-2-hydroxybenzoic acid

1142. PFKFB3 inhibitor for use in neuroprotection, wherein a PFKFB3 inhibitor is 4-(5-chloro-3-methyl-1-benzothiophene-2-sulfonamido)-2-hydroxybenzoic acid

1143. PFKFB3 inhibitor for use in neuroprotection, wherein a PFKFB3 inhibitor is 2-hydroxy-4-[3-(piperidin-1-yl)benzenesulfonamido]benzoic acid

1144. PFKFB3 inhibitor for use in neuroprotection wherein a PFKFB3 inhibitor is 4-(3-acetylbenzenesulfonamido)-2-hydroxybenzoic acid

1145. PFKFB3 inhibitor for use in neuroprotection wherein a PFKFB3 inhibitor is 4-(3-tert-butylbenzenesulfonamido)-2-hydroxybenzoic acid

1146. PFKFB3 inhibitor for use in neuroprotection, wherein a PFKFB3 inhibitor is 2-hydroxy-4-(4-phenylthiophene-2-sulfonamido)benzoic acid

1147. PFKFB3 inhibitor for use in neuroprotection, wherein a PFKFB3 inhibitor is 2-hydroxy-4-[3-(piperidine-1-carbonyl)benzenesulfonamido]benzoic acid

1148. PFKFB3 inhibitor for use in neuroprotection, wherein a PFKFB3 inhibitor is 2-hydroxy-4-[3-(methylcarbamoyl)benzenesulfonamido]benzoic acid

1149. PFKFB3 inhibitor for use in neuroprotection, wherein a PFKFB3 inhibitor is 2-hydroxy-4-{4′-methanesulfonyl-[1,1′-biphenyl]-3-sulfonamido}benzoic acid

1150. PFKFB3 inhibitor for use in neuroprotection, wherein a PFKFB3 inhibitor is 4-{3′-ethoxy-[1,1′-biphenyl]-3-sulfonamido}-2-hydroxybenzoic acid

1151. PFKFB3 inhibitor for use in neuroprotection, wherein a PFKFB3 inhibitor is 4-{3′-acetamido-[1,1′-biphenyl]-3-sulfonamido}-2-hydroxybenzoic acid

1152. PFKFB3 inhibitor for use in neuroprotection wherein a PFKFB3 inhibitor is 4-{3′,4′-dichloro-[1,1′-biphenyl]-3-sulfonamido}-2-hydroxybenzoic acid

1153. PFKFB3 inhibitor for use in neuroprotection, wherein a PFKFB3 inhibitor is 4-{3′-carbamoyl-[1,1′-biphenyl]-3-sulfonamido}-2-hydroxybenzoic acid

1154. PFKFB3 inhibitor for use in neuroprotection, wherein a PFKFB3 inhibitor is 4-{3′-cyano-[1,1′-biphenyl]-3-sulfonamido}-2-hydroxybenzoic acid

1155. PFKFB3 inhibitor for use in neuroprotection, wherein a PFKFB3 inhibitor is 2-hydroxy-4-{4′-nitro-[1,1′-biphenyl]-3-sulfonamido}benzoic acid

1156. PFKFB3 inhibitor for use in neuroprotection, wherein a PFKFB3 inhibitor is 2-hydroxy-4-[3′-(trifluoromethyl)-[1,1′-biphenyl]-3-sulfonamido]benzoic acid

1157. PFKFB3 inhibitor for use in neuroprotection, wherein a PFKFB3 inhibitor is 2-hydroxy-4-[4′-(methylsulfanyl)-[1,1′-biphenyl]-3-sulfonamido]benzoic acid

1158. PFKFB3 inhibitor for use in neuroprotection, wherein a PFKFB3 inhibitor is 2-hydroxy-4-[4′-(trifluoromethoxy)-[1,1′-biphenyl]-3-sulfonamido]benzoic acid

1159. PFKFB3 inhibitor for use in neuroprotection, wherein a PFKFB3 inhibitor is 4-{2′-acetyl-[1,1′-biphenyl]-3-sulfonamido}-2-hydroxybenzoic acid

1160. PFKFB3 inhibitor for use in neuroprotection, wherein a PFKFB3 inhibitor is 2-hydroxy-4-{4′-phenoxy-[1,1′-biphenyl]-3-sulfonamido}benzoic acid

1161. PFKFB3 inhibitor for use in neuroprotection, wherein a PFKFB3 inhibitor is 2-hydroxy-4-{4′-hydroxy-3′-methoxy-[1,1′-biphenyl]-3-sulfonamido}benzoic acid

1162. PFKFB3 inhibitor for use in neuroprotection, wherein a PFKFB3 inhibitor is 2-hydroxy-4-(3-methanesulfonylbenzenesulfonamido)benzoic acid

1163. PFKFB3 inhibitor for use in neuroprotection, wherein a PFKFB3 inhibitor is 4-[3-(1-benzofuran-2-yl)benzenesulfonamido]-2-hydroxybenzoic acid

1164. PFKFB3 inhibitor for use in neuroprotection, wherein a PFKFB3 inhibitor is 2-hydroxy-4-{4′-[(methoxycarbonyl)amino]-[1,1′-biphenyl]-3-sulfonamido}benzoic acid

1165. PFKFB3 inhibitor for use in neuroprotection wherein a PFKFB3 inhibitor is 4-(5-fluoro-2-methylbenzenesulfonamido)-2-hydroxybenzoic acid

1166. PFKFB3 inhibitor for use in neuroprotection wherein a PFKFB3 inhibitor is 4-(2-bromo-4-iodobenzenesulfonamido)-2-hydroxybenzoic acid

1167. PFKFB3 inhibitor for use in neuroprotection, wherein a PFKFB3 inhibitor is 2-hydroxy-4-(2,4,5-trichlorobenzenesulfonamido)benzoic acid

1168. PFKFB3 inhibitor for use in neuroprotection, wherein a PFKFB3 inhibitor is 2-hydroxy-4-[4-(1,3-oxazol-5-yl)benzenesulfonamido]benzoic acid

1169. PFKFB3 inhibitor for use in neuroprotection, wherein a PFKFB3 inhibitor is 4-(2,1,3-benzothiadiazole-4-sulfonamido)-2-hydroxybenzoic acid

1170. PFKFB3 inhibitor for use in neuroprotection, wherein a PFKFB3 inhibitor is 4-(2,1,3-benzoxadiazole-4-sulfonamido)-2-hydroxybenzoic acid

1171. PFKFB3 inhibitor for use in neuroprotection, wherein a PFKFB3 inhibitor is 4-{4′-chloro-[1,1′-biphenyl]-3-sulfonamido}-2-hydroxybenzoic acid

1172. PFKFB3 inhibitor for use in neuroprotection, wherein a PFKFB3 inhibitor is 2-hydroxy-4-[4′-(trifluoromethyl)-[1,1′-biphenyl]-3-sulfonamido]benzoic acid

1173. PFKFB3 inhibitor for use in neuroprotection, wherein a PFKFB3 inhibitor is 4-{4′-fluoro-[1,1′-biphenyl]-3-sulfonamido}-2-hydroxybenzoic acid

1174. PFKFB3 inhibitor for use in neuroprotection, wherein a PFKFB3 inhibitor is 4-{3′,5′-dichloro-[1,1′-biphenyl]-3-sulfonamido}-2-hydroxybenzoic acid

1175. PFKFB3 inhibitor for use in neuroprotection, wherein a PFKFB3 inhibitor is 2-hydroxy-4-{4′-methoxy-[1,1′-biphenyl]-3-sulfonamido}benzoic acid

1176. PFKFB3 inhibitor for use in neuroprotection, wherein a PFKFB3 inhibitor is 2-hydroxy-4-{4′-methyl-[1,1′-biphenyl]-3-sulfonamido}benzoic acid

1177. PFKFB3 inhibitor for use in neuroprotection, wherein a PFKFB3 inhibitor is 2-hydroxy-4-[3-(trifluoromethyl)benzenesulfonamido]benzoic acid

1178. PFKFB3 inhibitor for use in neuroprotection, wherein a PFKFB3 inhibitor is 4-(1-benzothiophene-2-sulfonamido)-2-hydroxybenzoic acid

1179. PFKFB3 inhibitor for use in neuroprotection, wherein a PFKFB3 inhibitor is 2-hydroxy-4-(5-methyl-1-benzothiophene-2-sulfonamido)benzoic acid

1180. PFKFB3 inhibitor for use in neuroprotection, wherein a PFKFB3 inhibitor is 2-hydroxy-4-(7-methoxy-3-methyl-1-benzothiophene-2-sulfonamido)benzoic acid

1181. PFKFB3 inhibitor for use in neuroprotection, wherein a PFKFB3 inhibitor is 2-hydroxy-4-(5-methoxy-3-methyl-1-benzothiophene-2-sulfonamido)benzoic acid

1182. PFKFB3 inhibitor for use in neuroprotection, wherein a PFKFB3 inhibitor is 2-hydroxy-4-[3-methyl-5-(propan-2-yl)-1-benzofuran-2-sulfonamido]benzoic acid

1183. PFKFB3 inhibitor for use in neuroprotection, wherein a PFKFB3 inhibitor is 4-(5-fluoro-3-methyl-1-benzothiophene-2-sulfonamido)-2-hydroxybenzoic acid

1184. PFKFB3 inhibitor for use in neuroprotection, wherein a PFKFB3 inhibitor is 4-[3-(2H-1,3-benzodioxol-5-yl)benzenesulfonamido]-2-hydroxybenzoic acid

1185. PFKFB3 inhibitor for use in neuroprotection, wherein a PFKFB3 inhibitor is 4-{2′,4′-difluoro-[1,1′-biphenyl]-3-sulfonamido}-2-hydroxybenzoic acid

1186. PFKFB3 inhibitor for use in neuroprotection, wherein a PFKFB3 inhibitor is 2-hydroxy-4-{2′-nitro-[1,1′-biphenyl]-3-sulfonamido}benzoic acid

1187. PFKFB3 inhibitor for use in neuroprotection, wherein a PFKFB3 inhibitor is 2-hydroxy-4-{4′-hydroxy-3′,5′-dimethyl-[1,1′-biphenyl]-3-sulfonamido}benzoic acid

1188. PFKFB3 inhibitor for use in neuroprotection, wherein a PFKFB3 inhibitor is 4-{4′-butyl-[1,1′-biphenyl]-3-sulfonamido}-2-hydroxybenzoic acid

1189. PFKFB3 inhibitor for use in neuroprotection, wherein a PFKFB3 inhibitor is 4-[4′-(ethanesulfonyl)-[1,1′-biphenyl]-3-sulfonamido]-2-hydroxybenzoic acid

1190. PFKFB3 inhibitor for use in neuroprotection, wherein a PFKFB3 inhibitor is 2-hydroxy-4-{4′-methoxy-3′-methyl-[1,1′-biphenyl]-3-sulfonamido}benzoic acid

1191. PFKFB3 inhibitor for use in neuroprotection, wherein a PFKFB3 inhibitor is 2-hydroxy-4-{3′-hydroxy-[1,1′-biphenyl]-3-sulfonamido}benzoic acid

1192. PFKFB3 inhibitor for use in neuroprotection, wherein a PFKFB3 inhibitor is 2-hydroxy-4-{3′-methanesulfonyl-[1,1′-biphenyl]-3-sulfonamido}benzoic acid

1193. PFKFB3 inhibitor for use in neuroprotection, wherein a PFKFB3 inhibitor is 4-[4′-(dimethylcarbamoyl)-[1,1′-biphenyl]-3-sulfonamido]-2-hydroxybenzoic acid

1194. PFKFB3 inhibitor for use in neuroprotection, wherein a PFKFB3 inhibitor is 4-{4′-ethyl-[1,1′-biphenyl]-3-sulfonamido}-2-hydroxybenzoic acid

1195. PFKFB3 inhibitor for use in neuroprotection, wherein a PFKFB3 inhibitor is 4-{4′-[bis(propan-2-yl)carbamoyl]-[1,1′-biphenyl]-3-sulfonamido}-2-hydroxybenzoic acid

1196. PFKFB3 inhibitor for use in neuroprotection, wherein a PFKFB3 inhibitor is 4-{4′-acetyl-[1,1′-biphenyl]-3-sulfonamido}-2-hydroxybenzoic acid

1197. PFKFB3 inhibitor for use in neuroprotection, wherein a PFKFB3 inhibitor is 4-{2′,3′-dimethoxy-[1,1′-biphenyl]-3-sulfonamido}-2-hydroxybenzoic acid

1198. PFKFB3 inhibitor for use in neuroprotection, wherein a PFKFB3 inhibitor is 4-{4′-fluoro-2′-methoxy-[1,1′-biphenyl]-3-sulfonamido}-2-hydroxybenzoic acid

1199. PFKFB3 inhibitor for use in neuroprotection, wherein a PFKFB3 inhibitor is 2-hydroxy-4-{2′,3′,6′-trifluoro-[1,1′-biphenyl]-3-sulfonamido}benzoic acid

1200. PFKFB3 inhibitor for use in neuroprotection, wherein a PFKFB3 inhibitor is 4-[4′-(2-carboxyethyl)-[1,1′-biphenyl]-3-sulfonamido]-2-hydroxybenzoic acid

1201. PFKFB3 inhibitor for use in neuroprotection, wherein a PFKFB3 inhibitor is 2-hydroxy-4-{3′-methyl-[1,1′-biphenyl]-3-sulfonamido}benzoic acid

1202. PFKFB3 inhibitor for use in neuroprotection, wherein a PFKFB3 inhibitor is 4-{3′,5′-difluoro-[1,1′-biphenyl]-3-sulfonamido}-2-hydroxybenzoic acid

1203. PFKFB3 inhibitor for use in neuroprotection, wherein a PFKFB3 inhibitor is 2-hydroxy-4-{4′-methoxy-3′,5′-dimethyl-[1,1′-biphenyl]-3-sulfonamido}benzoic acid

1204. PFKFB3 inhibitor for use in neuroprotection, wherein a PFKFB3 inhibitor is 2-hydroxy-4-{2′-methyl-[1,1′-biphenyl]-3-sulfonamido}benzoic acid

1205. PFKFB3 inhibitor for use in neuroprotection, wherein a PFKFB3 inhibitor is 2-hydroxy-4-[3-(2-propoxypyridin-3-yl)benzenesulfonamido]benzoic acid

1206. PFKFB3 inhibitor for use in neuroprotection, wherein a PFKFB3 inhibitor is 4-[3-(6-ethoxypyridin-3-yl)benzenesulfonamido]-2-hydroxybenzoic acid

1207. PFKFB3 inhibitor for use in neuroprotection, wherein a PFKFB3 inhibitor is 2-hydroxy-4-[4′-(propan-2-yloxy)-[1,1′-biphenyl]-3-sulfonamido]benzoic acid

1208. PFKFB3 inhibitor for use in neuroprotection, wherein a PFKFB3 inhibitor is 4-{4′-butoxy-[1,1′-biphenyl]-3-sulfonamido}-2-hydroxybenzoic acid

1209. PFKFB3 inhibitor for use in neuroprotection, wherein a PFKFB3 inhibitor is 4-{3′,4′-dimethoxy-[1,1′-biphenyl]-3-sulfonamido}-2-hydroxybenzoic acid

1210. PFKFB3 inhibitor for use in neuroprotection, wherein a PFKFB3 inhibitor is 2-hydroxy-4-[3-(6-methoxypyridin-3-yl)benzenesulfonamido]benzoic acid

1211. PFKFB3 inhibitor for use in neuroprotection, wherein a PFKFB3 inhibitor is 2-hydroxy-4-[3-(morpholin-4-yl)benzenesulfonamido]benzoic acid

1212. PFKFB3 inhibitor for use in neuroprotection, wherein a PFKFB3 inhibitor is 2-hydroxy-4-(5-phenyl-2,3-dihydro-1-benzofuran-7-sulfonamido)benzoic acid

1213. PFKFB3 inhibitor for use in neuroprotection wherein a PFKFB3 inhibitor is 4-(4-bromo-5-chlorothiophene-2-sulfonamido)-2-hydroxybenzoic acid

1214. PFKFB3 inhibitor for use in neuroprotection wherein a PFKFB3 inhibitor is 4-(5-bromo-6-chloropyridine-3-sulfonamido)-2-hydroxybenzoic acid

1215. PFKFB3 inhibitor for use in neuroprotection, wherein a PFKFB3 inhibitor is 4-(4,5-dichlorothiophene-2-sulfonamido)-2-hydroxybenzoic acid

1216. PFKFB3 inhibitor for use in neuroprotection, wherein a PFKFB3 inhibitor is 4-(3-bromothiophene-2-sulfonamido)-2-hydroxybenzoic acid

1217. PFKFB3 inhibitor for use in neuroprotection, wherein a PFKFB3 inhibitor is 4-(5-bromothiophene-2-sulfonamido)-2-hydroxybenzoic acid

1218. PFKFB3 inhibitor for use in neuroprotection wherein a PFKFB3 inhibitor is 4-(4-chloro-3-nitrobenzenesulfonamido)-2-hydroxybenzoic acid

1219. PFKFB3 inhibitor for use in neuroprotection wherein a PFKFB3 inhibitor is 4-(4-bromo-2,5-dichlorothiophene-3-sulfonamido)-2-hydroxybenzoic acid

1220. PFKFB3 inhibitor for use in neuroprotection, wherein a PFKFB3 inhibitor is 4-[3-(difluoromethoxy)benzenesulfonamido]-2-hydroxybenzoic acid

1221. PFKFB3 inhibitor for use in neuroprotection wherein a PFKFB3 inhibitor is 2-hydroxy-4-(3-methoxybenzenesulfonamido)benzoic acid

1222. PFKFB3 inhibitor for use in neuroprotection wherein a PFKFB3 inhibitor is 2-hydroxy-4-{5-[(phenylformamido)methyl]thiophene-2-sulfonamido}benzoic acid

1223. PFKFB3 inhibitor for use in neuroprotection, wherein a PFKFB3 inhibitor is 4-(3-chloro-4-methylbenzenesulfonamido)-2-hydroxybenzoic acid

1224. PFKFB3 inhibitor for use in neuroprotection, wherein a PFKFB3 inhibitor is 2-hydroxy-4-(4-methyl-3-nitrobenzenesulfonamido)benzoic acid

1225. PFKFB3 inhibitor for use in neuroprotection wherein a PFKFB3 inhibitor is 4-(4-bromobenzenesulfonamido)-2-hydroxybenzoic acid

1226. PFKFB3 inhibitor for use in neuroprotection wherein a PFKFB3 inhibitor is 4-(3-fluorobenzenesulfonamido)-2-hydroxybenzoic acid

1227. PFKFB3 inhibitor for use in neuroprotection, wherein a PFKFB3 inhibitor is 4-(2,5-dichlorothiophene-3-sulfonamido)-2-hydroxybenzoic acid

1228. PFKFB3 inhibitor for use in neuroprotection, wherein a PFKFB3 inhibitor is 2-hydroxy-4-(2,3,4-trichlorobenzenesulfonamido)benzoic acid

1229. PFKFB3 inhibitor for use in neuroprotection, wherein a PFKFB3 inhibitor is 2-hydroxy-4-(4-methylnaphthalene-1-sulfonamido)benzoic acid

1230. PFKFB3 inhibitor for use in neuroprotection, wherein a PFKFB3 inhibitor is 4-(4-fluoronaphthalene-1-sulfonamido)-2-hydroxybenzoic acid

1231. PFKFB3 inhibitor for use in neuroprotection, wherein a PFKFB3 inhibitor is 4-[5-(dimethylamino)naphthalene-1-sulfonamido]-2-hydroxybenzoic acid

1232. PFKFB3 inhibitor for use in neuroprotection, wherein a PFKFB3 inhibitor is 2-hydroxy-4-[3-(pyridin-4-yl)benzenesulfonamido]benzoic acid

1233. PFKFB3 inhibitor for use in neuroprotection, wherein a PFKFB3 inhibitor is 4-{4′-fluoro-3′-methyl-[1,1′-biphenyl]-3-sulfonamido}-2-hydroxybenzoic acid

1234. PFKFB3 inhibitor for use in neuroprotection, wherein a PFKFB3 inhibitor is 4-{3′-chloro-[1,1′-biphenyl]-3-sulfonamido}-2-hydroxybenzoic acid

1235. PFKFB3 inhibitor for use in neuroprotection, wherein a PFKFB3 inhibitor is 4-{4′-carbamoyl-[1,1′-biphenyl]-3-sulfonamido}-2-hydroxybenzoic acid

1236. PFKFB3 inhibitor for use in neuroprotection, wherein a PFKFB3 inhibitor is 4-{3′-fluoro-4′-methoxy-[1,1′-biphenyl]-3-sulfonamido}-2-hydroxybenzoic acid

1237. PFKFB3 inhibitor for use in neuroprotection, wherein a PFKFB3 inhibitor is 4-[6-chloro-5-(4-hydroxyphenyl)pyridine-3-sulfonamido]-2-hydroxybenzoic acid

1238. PFKFB3 inhibitor for use in neuroprotection, wherein a PFKFB3 inhibitor is 4-[6-chloro-5-(3-hydroxyphenyl)pyridine-3-sulfonamido]-2-hydroxybenzoic acid

1239. PFKFB3 inhibitor for use in neuroprotection, wherein a PFKFB3 inhibitor is 4-[5-(3-aminophenyl)-6-chloropyridine-3-sulfonamido]-2-hydroxybenzoic acid

1240. PFKFB3 inhibitor for use in neuroprotection, wherein a PFKFB3 inhibitor is 4-[6-chloro-5-(1H-pyrazol-4-yl)pyridine-3-sulfonamido]-2-hydroxybenzoic acid

1241. PFKFB3 inhibitor for use in neuroprotection, wherein a PFKFB3 inhibitor is 4-[6-chloro-5-(4-fluoro-3-methylphenyl)pyridine-3-sulfonamido]-2-hydroxybenzoic acid

1242. PFKFB3 inhibitor for use in neuroprotection, wherein a PFKFB3 inhibitor is 4-[6-chloro-5-(3-chlorophenyl)pyridine-3-sulfonamido]-2-hydroxybenzoic acid

1243. PFKFB3 inhibitor for use in neuroprotection, wherein a PFKFB3 inhibitor is 4-[6-chloro-5-(2-fluoro-3-methoxyphenyl)pyridine-3-sulfonamido]-2-hydroxybenzoic acid

1244. PFKFB3 inhibitor for use in neuroprotection, wherein a PFKFB3 inhibitor is 4-[5-(4-carbamoylphenyl)-6-chloropyridine-3-sulfonamido]-2-hydroxybenzoic acid

1245. PFKFB3 inhibitor for use in neuroprotection, wherein a PFKFB3 inhibitor is 4-[6-chloro-5-(3-fluorophenyl)pyridine-3-sulfonamido]-2-hydroxybenzoic acid

1246. PFKFB3 inhibitor for use in neuroprotection, wherein a PFKFB3 inhibitor is 4-[6-chloro-5-(3-fluoro-4-methoxyphenyl)pyridine-3-sulfonamido]-2-hydroxybenzoic acid

1247. PFKFB3 inhibitor for use in neuroprotection, wherein a PFKFB3 inhibitor is 4-[6-chloro-5-(quinolin-6-yl)pyridine-3-sulfonamido]-2-hydroxybenzoic acid

1248. PFKFB3 inhibitor for use in neuroprotection, wherein a PFKFB3 inhibitor is 4-[5-chloro-4-(pyridin-3-yl)thiophene-2-sulfonamido]-2-hydroxybenzoic acid

1249. PFKFB3 inhibitor for use in neuroprotection, wherein a PFKFB3 inhibitor is 4-[5-chloro-4-(3-hydroxyphenyl)thiophene-2-sulfonamido]-2-hydroxybenzoic acid

1250. PFKFB3 inhibitor for use in neuroprotection, wherein a PFKFB3 inhibitor is 4-[5-chloro-4-(4-hydroxy-3-methoxyphenyl)thiophene-2-sulfonamido]-2-hydroxybenzoic acid

1251. PFKFB3 inhibitor for use in neuroprotection, wherein a PFKFB3 inhibitor is 4-[5-chloro-4-(3-chlorophenyl)thiophene-2-sulfonamido]-2-hydroxybenzoic acid

1252. PFKFB3 inhibitor for use in neuroprotection, wherein a PFKFB3 inhibitor is 4-[4-(4-carbamoylphenyl)-5-chlorothiophene-2-sulfonamido]-2-hydroxybenzoic acid

1253. PFKFB3 inhibitor for use in neuroprotection, wherein a PFKFB3 inhibitor is 4-[5-chloro-4-(3-fluoro-4-methoxyphenyl)thiophene-2-sulfonamido]-2-hydroxybenzoic acid

1254. PFKFB3 inhibitor for use in neuroprotection, wherein a PFKFB3 inhibitor is 4-[4-(4-amino-3-methoxyphenyl)-5-chlorothiophene-2-sulfonamido]-2-hydroxybenzoic acid

1255. PFKFB3 inhibitor for use in neuroprotection, wherein a PFKFB3 inhibitor is 2-hydroxy-4-[2′-(methoxycarbonyl)-[1,1′-biphenyl]-3-sulfonamido]benzoic acid

1256. PFKFB3 inhibitor for use in neuroprotection, wherein a PFKFB3 inhibitor is 4-{5′-chloro-2′-methoxy-[1,1′-biphenyl]-3-sulfonamido}-2-hydroxybenzoic acid

1257. PFKFB3 inhibitor for use in neuroprotection, wherein a PFKFB3 inhibitor is 4-{2′,5′-difluoro-[1,1′-biphenyl]-3-sulfonamido}-2-hydroxybenzoic acid

1258. PFKFB3 inhibitor for use in neuroprotection, wherein a PFKFB3 inhibitor is 2-hydroxy-4-{2′-methoxy-[1,1′-biphenyl]-3-sulfonamido}benzoic acid

1259. PFKFB3 inhibitor for use in neuroprotection, wherein a PFKFB3 inhibitor is 4-{2′-fluoro-3′-methoxy-[1,1′-biphenyl]-3-sulfonamido}-2-hydroxybenzoic acid

1260. PFKFB3 inhibitor for use in neuroprotection, wherein a PFKFB3 inhibitor is 2-hydroxy-4-{2′-hydroxy-[1,1′-biphenyl]-3-sulfonamido}benzoic acid

1261. PFKFB3 inhibitor for use in neuroprotection, wherein a PFKFB3 inhibitor is 4-{2′-amino-[1,1′-biphenyl]-3-sulfonamido}-2-hydroxybenzoic acid

1262. PFKFB3 inhibitor for use in neuroprotection, wherein a PFKFB3 inhibitor is 4-{5′-fluoro-2′-methoxy-[1,1′-biphenyl]-3-sulfonamido}-2-hydroxybenzoic acid

1263. PFKFB3 inhibitor for use in neuroprotection, wherein a PFKFB3 inhibitor is 4-[5-chloro-4-(5-chloro-2-methoxyphenyl)thiophene-2-sulfonamido]-2-hydroxybenzoic acid

1264. PFKFB3 inhibitor for use in neuroprotection, wherein a PFKFB3 inhibitor is 4-[5-chloro-4-(2,5-difluorophenyl)thiophene-2-sulfonamido]-2-hydroxybenzoic acid

1265. PFKFB3 inhibitor for use in neuroprotection, wherein a PFKFB3 inhibitor is 4-[5-chloro-4-(2-methoxyphenyl)thiophene-2-sulfonamido]-2-hydroxybenzoic acid

1266. PFKFB3 inhibitor for use in neuroprotection, wherein a PFKFB3 inhibitor is 4-[5-chloro-4-(2-fluoro-3-methoxyphenyl)thiophene-2-sulfonamido]-2-hydroxybenzoic acid

1267. PFKFB3 inhibitor for use in neuroprotection, wherein a PFKFB3 inhibitor is 4-[4-(2-aminophenyl)-5-chlorothiophene-2-sulfonamido]-2-hydroxybenzoic acid

1268. PFKFB3 inhibitor for use in neuroprotection, wherein a PFKFB3 inhibitor is 4-[5-chloro-4-(5-fluoro-2-methoxyphenyl)thiophene-2-sulfonamido]-2-hydroxybenzoic acid

1269. PFKFB3 inhibitor for use in neuroprotection, wherein a PFKFB3 inhibitor is 4-[5-chloro-4-(2-hydroxyphenyl)thiophene-2-sulfonamido]-2-hydroxybenzoic acid

1270. PFKFB3 inhibitor for use in neuroprotection, wherein a PFKFB3 inhibitor is 4-(2,3-dichlorobenzenesulfonamido)-2-hydroxybenzoic acid

1271. PFKFB3 inhibitor for use in neuroprotection wherein a PFKFB3 inhibitor is 4-(3-chloro-4-fluorobenzenesulfonamido)-2-hydroxybenzoic acid

1272. PFKFB3 inhibitor for use in neuroprotection wherein a PFKFB3 inhibitor is 4-(4-bromo-2,5-difluorobenzenesulfonamido)-2-hydroxybenzoic acid

1273. PFKFB3 inhibitor for use in neuroprotection wherein a PFKFB3 inhibitor is 2-hydroxy-4-(3-methylbenzenesulfonamido)benzoic acid

1274. PFKFB3 inhibitor for use in neuroprotection, wherein a PFKFB3 inhibitor is 4-{[1,1′-biphenyl]-4-sulfonamido}-2-hydroxybenzoic acid

1275. PFKFB3 inhibitor for use in neuroprotection, wherein a PFKFB3 inhibitor is 4-(1-benzothiophene-3-sulfonamido)-2-hydroxybenzoic acid

1276. PFKFB3 inhibitor for use in neuroprotection wherein a PFKFB3 inhibitor is 4-(2,5-dichloro-4-methylthiophene-3-sulfonamido)-2-hydroxybenzoic acid

1277. PFKFB3 inhibitor for use in neuroprotection wherein a PFKFB3 inhibitor is 2-hydroxy-4-(2,4,5-trichlorothiophene-3-sulfonamido)benzoic acid

1278. PFKFB3 inhibitor for use in neuroprotection wherein a PFKFB3 inhibitor is 4-(2-chloro-6-methylbenzenesulfonamido)-2-hydroxybenzoic acid

1279. PFKFB3 inhibitor for use in neuroprotection wherein a PFKFB3 inhibitor is 2-hydroxy-4-[3-(trifluoromethoxy)benzenesulfonamido]benzoic acid

1280. PFKFB3 inhibitor for use in neuroprotection, wherein a PFKFB3 inhibitor is 4-(1-benzofuran-2-sulfonamido)-2-hydroxybenzoic acid

1281. PFKFB3 inhibitor for use in neuroprotection, wherein a PFKFB3 inhibitor is 2-hydroxy-4-[5-methyl-2-(trifluoromethyl)furan-3-sulfonamido]benzoic acid

1282. PFKFB3 inhibitor for use in neuroprotection, wherein a PFKFB3 inhibitor is 4-(3-chloro-2-methylbenzenesulfonamido)-2-hydroxybenzoic acid

1283. PFKFB3 inhibitor for use in neuroprotection, wherein a PFKFB3 inhibitor is 2-hydroxy-4-[3-methyl-5-(propan-2-yl)-1-benzothiophene-2-sulfonamido]benzoic acid

1284. PFKFB3 inhibitor for use in neuroprotection, wherein a PFKFB3 inhibitor is 4-[4-(2,3-dihydro-1-benzofuran-5-yl)thiophene-2-sulfonamido]-2-hydroxybenzoic acid

1285. PFKFB3 inhibitor for use in neuroprotection wherein a PFKFB3 inhibitor is 2-hydroxy-4-[3-(1-hydroxyethyl)benzenesulfonamido]benzoic acid

1286. PFKFB3 inhibitor for use in neuroprotection wherein a PFKFB3 inhibitor is 2-hydroxy-4-(3-hydroxybenzenesulfonamido)benzoic acid

1287. PFKFB3 inhibitor for use in neuroprotection wherein a PFKFB3 inhibitor is 2-hydroxy-4-(2-hydroxybenzenesulfonamido)benzoic acid

1288. PFKFB3 inhibitor for use in neuroprotection wherein a PFKFB3 inhibitor is 4-[(4-chlorophenyl)methanesulfonamido]-2-hydroxybenzoic acid

1289. PFKFB3 inhibitor for use in neuroprotection wherein a PFKFB3 inhibitor is 4-[(3-bromophenyl)methanesulfonamido]-2-hydroxybenzoic acid

1290. PFKFB3 inhibitor for use in neuroprotection wherein a PFKFB3 inhibitor is 4-[(4-bromophenyl)methanesulfonamido]-2-hydroxybenzoic acid

1291. PFKFB3 inhibitor for use in neuroprotection, wherein a PFKFB3 inhibitor is 2-hydroxy-4-({2′-hydroxy-[1,1′-biphenyl]-4-yl}methanesulfonamido)benzoic acid

1292. PFKFB3 inhibitor for use in neuroprotection, wherein a PFKFB3 inhibitor is 4-{[4-(2,3-dihydro-1-benzofuran-5-yl)phenyl]methanesulfonamido}-2-hydroxybenzoic acid

1293. PFKFB3 inhibitor for use in neuroprotection, wherein a PFKFB3 inhibitor is 4-({2′,5′-difluoro-[1,1′-biphenyl]-4-yl}methanesulfonamido)-2-hydroxybenzoic acid

1294. PFKFB3 inhibitor for use in neuroprotection wherein a PFKFB3 inhibitor is 4-({[1,1′-biphenyl]-4-yl}methanesulfonamido)-2-hydroxybenzoic acid

1295. PFKFB3 inhibitor for use in neuroprotection wherein a PFKFB3 inhibitor is 4-{[3-(2,3-dihydro-1-benzofuran-5-yl)phenyl]methanesulfonamido}-2-hydroxybenzoic acid

1296. PFKFB3 inhibitor for use in neuroprotection, wherein a PFKFB3 inhibitor is 4-({2′,5′-difluoro-[1,1′-biphenyl]-3-yl}methanesulfonamido)-2-hydroxybenzoic acid

1297. PFKFB3 inhibitor for use in neuroprotection, wherein a PFKFB3 inhibitor is 4-(3,5-dibromothiophene-2-sulfonamido)-2-hydroxybenzoic acid

1298. PFKFB3 inhibitor for use in neuroprotection, wherein a PFKFB3 inhibitor is 4-(3,4-dibromothiophene-2-sulfonamido)-2-hydroxybenzoic acid

1299. PFKFB3 inhibitor for use in neuroprotection, wherein a PFKFB3 inhibitor is 4-(4,5-dibromothiophene-2-sulfonamido)-2-hydroxybenzoic acid

1300. PFKFB3 inhibitor for use in neuroprotection wherein a PFKFB3 inhibitor is 4-(5-bromo-4-methylthiophene-2-sulfonamido)-2-hydroxybenzoic acid

1301. PFKFB3 inhibitor for use in neuroprotection wherein a PFKFB3 inhibitor is 4-(5-chloro-4-methylthiophene-2-sulfonamido)-2-hydroxybenzoic acid

1302. PFKFB3 inhibitor for use in neuroprotection, wherein a PFKFB3 inhibitor is 4-(3,5-dichlorobenzenesulfonamido)-2-hydroxybenzoic acid

1303. PFKFB3 inhibitor for use in neuroprotection, wherein a PFKFB3 inhibitor is 4-[3-bromo-5-(trifluoromethyl)benzenesulfonamido]-2-hydroxybenzoic acid

1304. PFKFB3 inhibitor for use in neuroprotection, wherein a PFKFB3 inhibitor is 4-[2′,5′-difluoro-5-(trifluoromethyl)-[1,1′-biphenyl]-3-sulfonamido]-2-hydroxybenzoic acid

1305. PFKFB3 inhibitor for use in neuroprotection, wherein a PFKFB3 inhibitor is 4-[3-(2,3-dihydro-1-benzofuran-5-yl)-5-(trifluoromethyl)benzenesulfonamido]-2-hydroxybenzoic acid

1306. PFKFB3 inhibitor for use in neuroprotection, wherein a PFKFB3 inhibitor is 2-hydroxy-4-[2′-hydroxy-5-(trifluoromethyl)-[1,1′-biphenyl]-3-sulfonamido]benzoic acid

1307. PFKFB3 inhibitor for use in neuroprotection wherein a PFKFB3 inhibitor is 4-(3-chloro-2-fluorobenzenesulfonamido)-2-hydroxybenzoic acid

1308. PFKFB3 inhibitor for use in neuroprotection wherein a PFKFB3 inhibitor is 4-(5-chloro-2-fluorobenzenesulfonamido)-2-hydroxybenzoic acid

1309. PFKFB3 inhibitor for use in neuroprotection, wherein a PFKFB3 inhibitor is 4-(2,5-dimethylfuran-3-sulfonamido)-2-hydroxybenzoic acid

1310. PFKFB3 inhibitor for use in neuroprotection, wherein a PFKFB3 inhibitor is 4-[5-(2,5-difluorophenyl)thiophene-2-sulfonamido]-2-hydroxybenzoic acid

1311. PFKFB3 inhibitor for use in neuroprotection, wherein a PFKFB3 inhibitor is 4-[5-(2,3-dihydro-1-benzofuran-5-yl)thiophene-2-sulfonamido]-2-hydroxybenzoic acid

1312. PFKFB3 inhibitor for use in neuroprotection wherein a PFKFB3 inhibitor is 2-hydroxy-4-(5-phenylthiophene-2-sulfonamido)benzoic acid

1313. PFKFB3 inhibitor for use in neuroprotection wherein a PFKFB3 inhibitor is 2-hydroxy-4-[5-(2-hydroxyphenyl)thiophene-2-sulfonamido]benzoic acid

1314. PFKFB3 inhibitor for use in neuroprotection wherein a PFKFB3 inhibitor is 2-hydroxy-4-(4-phenoxybenzenesulfonamido)benzoic acid

1315. PFKFB3 inhibitor for use in neuroprotection, wherein a PFKFB3 inhibitor is 4-(2,5-dimethylthiophene-3-sulfonamido)-2-hydroxybenzoic acid

1316. PFKFB3 inhibitor for use in neuroprotection, wherein a PFKFB3 inhibitor is 4-(4-chlorobenzenesulfonamido)-2-hydroxybenzoic acid

1317. PFKFB3 inhibitor for use in neuroprotection, wherein a PFKFB3 inhibitor is 4-benzenesulfonamido-2-hydroxybenzoic acid

1318. PFKFB3 inhibitor for use in neuroprotection, wherein a PFKFB3 inhibitor is 2-hydroxy-4-(3-nitrobenzenesulfonamido)benzoic acid

1319. PFKFB3 inhibitor for use in neuroprotection, wherein a PFKFB3 inhibitor is 2-hydroxy-4-(naphthalene-2-sulfonamido)benzoic acid

1320. PFKFB3 inhibitor for use in neuroprotection wherein a PFKFB3 inhibitor is 4-(3-carboxybenzenesulfonamido)-2-hydroxybenzoic acid

1321. PFKFB3 inhibitor for use in neuroprotection wherein a PFKFB3 inhibitor is 4-(4-carboxybenzenesulfonamido)-2-hydroxybenzoic acid

1322. PFKFB3 inhibitor for use in neuroprotection wherein a PFKFB3 inhibitor is 4-(2,5-dichlorobenzenesulfonamido)-2-hydroxybenzoic acid

1323. PFKFB3 inhibitor for use in neuroprotection wherein a PFKFB3 inhibitor is 4-(3-chlorobenzenesulfonamido)-2-hydroxybenzoic acid

1324. PFKFB3 inhibitor for use in neuroprotection, wherein a PFKFB3 inhibitor is 4-(3-bromo-5-chlorothiophene-2-sulfonamido)-2-hydroxybenzoic acid

1325. PFKFB3 inhibitor for use in neuroprotection, wherein a PFKFB3 inhibitor is 4-(4-bromothiophene-3-sulfonamido)-2-hydroxybenzoic acid

1326. PFKFB3 inhibitor for use in neuroprotection, wherein a PFKFB3 inhibitor is N-(4-{[3-cyano-1-(2-methylpropyl)-1H-indol-5-yl]oxy}phenyl)pyrrolidine-2-carboxamide

1327. PFKFB3 inhibitor for use in neuroprotection, wherein a PFKFB3 inhibitor is N-[(S)-(1-methyl-1H-1,2,3-triazol-5-yl)(1-methyl-1H-pyrazol-3-yl)methyl]-8-(1-methyl-1H-indol-6-yl)quinoxalin-6-amine

1328. PFKFB3 inhibitor for use in neuroprotection, wherein a PFKFB3 inhibitor is N-[(R)-(1-methyl-1H-1,2,3-triazol-5-yl)(1-methyl-1H-pyrazol-3-yl)methyl]-8-(1-methyl-1H-indol-6-yl)quinoxalin-6-amine

1329. PFKFB3 inhibitor for use in neuroprotection, wherein a PFKFB3 inhibitor is N-[(R)-(1-methyl-1H-pyrazol-4-yl)(pyridin-3-yl)methyl]-8-{1-methyl-1H-pyrrolo[3,2-b]pyridin-6-yl}quinoxalin-6-amine

1330. PFKFB3 inhibitor for use in neuroprotection, wherein a PFKFB3 inhibitor is N-[(S)-(1-methyl-1H-pyrazol-4-yl)(pyridin-3-yl)methyl]-8-{1-methyl-1H-pyrrolo[3,2-b]pyridin-6-yl}quinoxalin-6-amine

1331. PFKFB3 inhibitor for use in neuroprotection, wherein a PFKFB3 inhibitor is 8-(3-methyl-1-benzofuran-5-yl)-N-[(R)-(1-methyl-1H-1,2,3-triazol-5-yl)(1-methyl-1H-pyrazol-4-yl)methyl]quinoxalin-6-amine

1332. PFKFB3 inhibitor for use in neuroprotection, wherein a PFKFB3 inhibitor is 8-(3-methyl-1-benzofuran-5-yl)-N-[(S)-(1-methyl-1H-1,2,3-triazol-5-yl)(1-methyl-1H-pyrazol-4-yl)methyl]quinoxalin-6-amine

1333. PFKFB3 inhibitor for use in neuroprotection, wherein a PFKFB3 inhibitor is N-[(1S)-2-methyl-1-(pyridin-3-yl)propyl]-8-(1-methyl-1H-indol-6-yl)quinoxalin-6-amine

1334. PFKFB3 inhibitor for use in neuroprotection, wherein a PFKFB3 inhibitor is N-[(1R)-2-methyl-1-(pyridin-3-yl)propyl]-8-(1-methyl-1H-indol-6-yl)quinoxalin-6-amine

1335. PFKFB3 inhibitor for use in neuroprotection, wherein a PFKFB3 inhibitor is 8-(1-methyl-1H-indol-6-yl)-N-[(R)-(1-methyl-1H-pyrazol-4-yl)(pyridin-3-yl)methyl]quinoxalin-6-amine

1336. PFKFB3 inhibitor for use in neuroprotection, wherein a PFKFB3 inhibitor is 8-(1-methyl-1H-indol-6-yl)-N-[(S)-(1-methyl-1H-pyrazol-4-yl)(pyridin-3-yl)methyl]quinoxalin-6-amine

1337. PFKFB3 inhibitor for use in neuroprotection, wherein a PFKFB3 inhibitor is N-[(R)-(1-methyl-1H-imidazol-2-yl)(1-methyl-1H-pyrazol-4-yl)methyl]-8-(1-methyl-1H-indol-6-yl)quinoxalin-6-amine

1338. PFKFB3 inhibitor for use in neuroprotection, wherein a PFKFB3 inhibitor is N-[(S)-(1-methyl-1H-imidazol-2-yl)(1-methyl-1H-pyrazol-4-yl)methyl]-8-(1-methyl-1H-indol-6-yl)quinoxalin-6-amine

1339. PFKFB3 inhibitor for use in neuroprotection, wherein a PFKFB3 inhibitor is N-[(R)-(1-methyl-1H-imidazol-5-yl)(1-methyl-1H-pyrazol-4-yl)methyl]-8-(1-methyl-1H-indol-6-yl)quinoxalin-6-amine

1340. PFKFB3 inhibitor for use in neuroprotection, wherein a PFKFB3 inhibitor is N-[(S)-(1-methyl-1H-imidazol-5-yl)(1-methyl-1H-pyrazol-4-yl)methyl]-8-(1-methyl-1H-indol-6-yl)quinoxalin-6-amine

1341. PFKFB3 inhibitor for use in neuroprotection, wherein a PFKFB3 inhibitor is N-[(R)-(1-methyl-1H-1,2,3-triazol-5-yl)(1-methyl-1H-pyrazol-4-yl)methyl]-8-(1-methyl-1H-indol-6-yl)quinoxalin-6-amine

1342. PFKFB3 inhibitor for use in neuroprotection, wherein a PFKFB3 inhibitor is N-[(S)-(1-methyl-1H-1,2,3-triazol-5-yl)(1-methyl-1H-pyrazol-4-yl)methyl]-8-(1-methyl-1H-indol-6-yl)quinoxalin-6-amine

1343. PFKFB3 inhibitor for use in neuroprotection, wherein a PFKFB3 inhibitor is N-[(1-methyl-1H-1,2,3-triazol-5-yl)(1-methyl-1H-pyrazol-3-yl)methyl]-8-(1-methyl-1H-indol-6-yl)quinoxalin-6-amine

1344. PFKFB3 inhibitor for use in neuroprotection, wherein a PFKFB3 inhibitor is N-[(1-methyl-1H-pyrazol-4-yl)(pyridin-3-yl)methyl]-8-{1-methyl-1H-pyrrolo[3,2-b]pyridin-6-yl}quinoxalin-6-amine

1345. PFKFB3 inhibitor for use in neuroprotection, wherein a PFKFB3 inhibitor is 8-(3-methyl-1-benzofuran-5-yl)-N-[(1-methyl-1H-1,2,3-triazol-5-yl)(1-methyl-1H-pyrazol-4-yl)methyl]quinoxalin-6-amine

1346. PFKFB3 inhibitor for use in neuroprotection, wherein a PFKFB3 inhibitor is N-[2-methyl-1-(pyridin-3-yl)propyl]-8-(1-methyl-1H-indol-6-yl)quinoxalin-6-amine

1347. PFKFB3 inhibitor for use in neuroprotection, wherein a PFKFB3 inhibitor is 8-(1-methyl-1H-indol-6-yl)-N-[(1-methyl-1H-pyrazol-4-yl)(pyridin-3-yl)methyl]quinoxalin-6-amine

1348. PFKFB3 inhibitor for use in neuroprotection, wherein a PFKFB3 inhibitor is N-[(1-methyl-1H-imidazol-2-yl)(1-methyl-1H-pyrazol-4-yl)methyl]-8-(1-methyl-1H-indol-6-yl)quinoxalin-6-amine

1349. PFKFB3 inhibitor for use in neuroprotection, wherein a PFKFB3 inhibitor is N-[(1-methyl-1H-imidazol-5-yl)(1-methyl-1H-pyrazol-4-yl)methyl]-8-(1-methyl-1H-indol-6-yl)quinoxalin-6-amine

1350. PFKFB3 inhibitor for use in neuroprotection, wherein a PFKFB3 inhibitor is N-[(1-methyl-1H-1,2,3-triazol-5-yl)(1-methyl-1H-pyrazol-4-yl)methyl]-8-(1-methyl-1H-indol-6-yl)quinoxalin-6-amine

1351. PFKFB3 inhibitor for use in neuroprotection, wherein a PFKFB3 inhibitor is N-[(R)-(6-methoxypyridazin-3-yl)(pyridin-3-yl)methyl]-8-(1-methyl-1H-indol-6-yl)quinoxalin-6-amine

1352. PFKFB3 inhibitor for use in neuroprotection, wherein a PFKFB3 inhibitor is N-[(S)-(6-methoxypyridazin-3-yl)(pyridin-3-yl)methyl]-8-(1-methyl-1H-indol-6-yl)quinoxalin-6-amine

1353. PFKFB3 inhibitor for use in neuroprotection, wherein a PFKFB3 inhibitor is N-[(6-methoxypyridazin-3-yl)(pyridin-3-yl)methyl]-8-(1-methyl-1H-indol-6-yl)quinoxalin-6-amine

1354. PFKFB3 inhibitor for use in neuroprotection, wherein a PFKFB3 inhibitor is 6-[(R)-{[8-(1-methyl-1H-indol-6-yl)quinoxalin-6-yl]amino}(pyridin-3-yl)methyl]-2,3-dihydropyridazin-3-one

1355. PFKFB3 inhibitor for use in neuroprotection, wherein a PFKFB3 inhibitor is 6-[(S)-{[8-(1-methyl-1H-indol-6-yl)quinoxalin-6-yl]amino}(pyridin-3-yl)methyl]-2,3-dihydropyridazin-3-one

1356. PFKFB3 inhibitor for use in neuroprotection, wherein a PFKFB3 inhibitor is 6-({[8-(1-methyl-1H-indol-6-yl)quinoxalin-6-yl]amino}(pyridin-3-yl)methyl)-2,3-dihydropyridazin-3-one

1357. PFKFB3 inhibitor for use in neuroprotection, wherein a PFKFB3 inhibitor is 3-{[8-(4-bromophenyl)quinoxalin-6-yl]amino}-N-(1-methylpyrrolidin-3-yl)pyridine-4-carboxamide

1358. PFKFB3 inhibitor for use in neuroprotection, wherein a PFKFB3 inhibitor is 3-{[8-(2-amino-1,3-benzothiazol-5-yl)quinoxalin-6-yl]amino}-N-(1-methylpyrrolidin-3-yl)pyridine-4-carboxamide

1359. PFKFB3 inhibitor for use in neuroprotection, wherein a PFKFB3 inhibitor is N-(4-methanesulfonylpyridin-3-yl)-8-[4-(pentafluoro- [symbol] -sulfanyl)phenyl]quinoxalin-6-amine

1360. PFKFB3 inhibitor for use in neuroprotection, wherein a PFKFB3 inhibitor is 8-(4-bromo-2-methylphenyl)-N-(4-methanesulfonylpyridin-3-yl)quinoxalin-6-amine

1361. PFKFB3 inhibitor for use in neuroprotection, wherein a PFKFB3 inhibitor is 8-(4-bromo-3-fluorophenyl)-N-(4-methanesulfonylpyridin-3-yl)quinoxalin-6-amine

1362. PFKFB3 inhibitor for use in neuroprotection, wherein a PFKFB3 inhibitor is N-(4-methanesulfonylpyridin-3-yl)-8-[3-(methylsulfanyl)phenyl]quinoxalin-6-amine

1363. PFKFB3 inhibitor for use in neuroprotection, wherein a PFKFB3 inhibitor is 2-{[8-(1-methyl-1H-indol-6-yl)quinoxalin-6-yl]amino}-N-(oxan-3-yl)benzene-1-sulfonamide

1364. PFKFB3 inhibitor for use in neuroprotection, wherein a PFKFB3 inhibitor is 2-{[8-(1-methyl-1H-indol-6-yl)quinoxalin-6-yl]amino}-N-(1-methylpiperidin-3-yl)benzene-1-sulfonamide

1365. PFKFB3 inhibitor for use in neuroprotection, wherein a PFKFB3 inhibitor is 3-{[8-(1-methyl-1H-indol-6-yl)quinoxalin-6-yl]amino}-N-(pyridin-3-yl)pyridine-4-carboxamide

1366. PFKFB3 inhibitor for use in neuroprotection, wherein a PFKFB3 inhibitor is N-(1-acetylazetidin-3-yl)-3-{[8-(3-methyl-1-benzothiophen-5-yl)quinoxalin-6-yl]amino}pyridine-4-carboxamide

1367. PFKFB3 inhibitor for use in neuroprotection, wherein a PFKFB3 inhibitor is 3-{[8-(3-methyl-1-benzothiophen-5-yl)quinoxalin-6-yl]amino}pyridine-4-carboxamide

1368. PFKFB3 inhibitor for use in neuroprotection, wherein a PFKFB3 inhibitor is N-(5-bromopyrimidin-4-yl)-8-(1-methyl-1H-indol-6-yl)quinoxalin-6-amine

1369. PFKFB3 inhibitor for use in neuroprotection, wherein a PFKFB3 inhibitor is 8-(2-amino-1-benzothiophen-5-yl)-N-(4-methanesulfonylpyridin-3-yl)quinoxalin-6-amine

1370. PFKFB3 inhibitor for use in neuroprotection, wherein a PFKFB3 inhibitor is N-(4-methanesulfonylpyridin-3-yl)-8-[2-(methylamino)-1,3-benzothiazol-5-yl]quinoxalin-6-amine

1371. PFKFB3 inhibitor for use in neuroprotection, wherein a PFKFB3 inhibitor is 8-[2-(dimethylamino)-1,3-benzothiazol-5-yl]-N-(4-methanesulfonylpyridin-3-yl)quinoxalin-6-amine

1372. PFKFB3 inhibitor for use in neuroprotection, wherein a PFKFB3 inhibitor is N-(5-{7-[(4-methanesulfonylpyridin-3-yl)amino]quinoxalin-5-yl}-1-benzothiophen-2-yl)acetamide

1373. PFKFB3 inhibitor for use in neuroprotection, wherein a PFKFB3 inhibitor is 2-{[8-(3-methyl-1-benzothiophen-5-yl)quinoxalin-6-yl]amino}-N-(1-methylpyrrolidin-3-yl)benzene-1-sulfonamide

1374. PFKFB3 inhibitor for use in neuroprotection, wherein a PFKFB3 inhibitor is 3-{[8-(1-methyl-1H-indol-6-yl)quinoxalin-6-yl]oxy}pyridine-4-carboxylic acid

1375. PFKFB3 inhibitor for use in neuroprotection, wherein a PFKFB3 inhibitor is 3-{[8-(4-fluoro-1-methyl-1H-indol-6-yl)quinoxalin-6-yl]amino}-N-(1-methylpyrrolidin-3-yl)pyridine-4-carboxamide

1376. PFKFB3 inhibitor for use in neuroprotection, wherein a PFKFB3 inhibitor is 8-(1,5-dimethyl-1H-indol-6-yl)-N-(4-methanesulfonylpyridin-3-yl)quinoxalin-6-amine

1377. PFKFB3 inhibitor for use in neuroprotection, wherein a PFKFB3 inhibitor is 3-{[8-(3-methyl-1-benzothiophen-5-yl)quinoxalin-6-yl]amino}-N-[(3R)-1-methylpyrrolidin-3-yl]pyridine-4-carboxamide

1378. PFKFB3 inhibitor for use in neuroprotection, wherein a PFKFB3 inhibitor is 3-{[8-(3-methyl-1-benzothiophen-5-yl)quinoxalin-6-yl]amino}-N-[(3S)-1-methylpyrrolidin-3-yl]pyridine-4-carboxamide

1379. PFKFB3 inhibitor for use in neuroprotection, wherein a PFKFB3 inhibitor is 8-(3,5-diethylphenyl)-N-(4-methanesulfonylpyridin-3-yl)quinoxalin-6-amine

1380. PFKFB3 inhibitor for use in neuroprotection, wherein a PFKFB3 inhibitor is N-(2-methanesulfonylphenyl)-8-(3-methyl-1-benzothiophen-5-yl)quinoxalin-6-amine

1381. PFKFB3 inhibitor for use in neuroprotection, wherein a PFKFB3 inhibitor is 8-(2-amino-1,3-benzothiazol-5-yl)-N-(2-methanesulfonylphenyl)quinoxalin-6-amine

1382. PFKFB3 inhibitor for use in neuroprotection, wherein a PFKFB3 inhibitor is 8-(1,4-dimethyl-1H-indol-6-yl)-N-(4-methanesulfonylpyridin-3-yl)quinoxalin-6-amine

1383. PFKFB3 inhibitor for use in neuroprotection, wherein a PFKFB3 inhibitor is 8-(4-fluorophenyl)-N-(4-methanesulfonylpyridin-3-yl)quinoxalin-6-amine

1384. PFKFB3 inhibitor for use in neuroprotection, wherein a PFKFB3 inhibitor is N-methyl-2-{[8-(1-methyl-1H-indol-6-yl)quinoxalin-6-yl]amino}-N-[(pyrimidin-5-yl)methyl]benzene-1-sulfonamide

1385. PFKFB3 inhibitor for use in neuroprotection, wherein a PFKFB3 inhibitor is 8-(4-amino-3-fluorophenyl)-N-(4-methanesulfonylpyridin-3-yl)quinoxalin-6-amine

1386. PFKFB3 inhibitor for use in neuroprotection, wherein a PFKFB3 inhibitor is N-(4-methanesulfonylpyridin-3-yl)-8-[4-(trifluoromethyl)phenyl]quinoxalin-6-amine

1387. PFKFB3 inhibitor for use in neuroprotection, wherein a PFKFB3 inhibitor is N-(4-methanesulfonylpyridin-3-yl)-8-(1-propyl-1H-indol-6-yl)quinoxalin-6-amine

1388. PFKFB3 inhibitor for use in neuroprotection, wherein a PFKFB3 inhibitor is 2-{[8-(1-methyl-1H-indol-6-yl)quinoxalin-6-yl]amino}-N-(1-methylpyrrolidin-3-yl)benzamide

1389. PFKFB3 inhibitor for use in neuroprotection, wherein a PFKFB3 inhibitor is N-methyl-3-{[8-(1-methyl-1H-indol-6-yl)quinoxalin-6-yl]amino}-N-(1-methylpyrrolidin-3-yl)pyridine-4-carboxamide

1390. PFKFB3 inhibitor for use in neuroprotection, wherein a PFKFB3 inhibitor is 3-{[8-(1-methyl-1H-indol-6-yl)quinoxalin-6-yl]amino}-N-(1-methylazetidin-3-yl)pyridine-4-carboxamide

1391. PFKFB3 inhibitor for use in neuroprotection, wherein a PFKFB3 inhibitor is 3-{[8-(1-methyl-1H-indol-6-yl)quinoxalin-6-yl]amino}pyridine-4-carboxylic acid

1392. PFKFB3 inhibitor for use in neuroprotection, wherein a PFKFB3 inhibitor is 2-{[8-(1-methyl-1H-indol-6-yl)quinoxalin-6-yl]amino}benzoic acid

1393. PFKFB3 inhibitor for use in neuroprotection, wherein a PFKFB3 inhibitor is N-{2-[(dimethylamino)methyl]phenyl}-8-(1-methyl-1H-indol-6-yl)quinoxalin-6-amine

1394. PFKFB3 inhibitor for use in neuroprotection, wherein a PFKFB3 inhibitor is N-{4-[(dimethylamino)methyl]pyridin-3-yl}-8-(1-methyl-1H-indol-6-yl)quinoxalin-6-amine

1395. PFKFB3 inhibitor for use in neuroprotection, wherein a PFKFB3 inhibitor is 8-(2-amino-1,3-benzothiazol-6-yl)-N-(4-methanesulfonylpyridin-3-yl)quinoxalin-6-amine

1396. PFKFB3 inhibitor for use in neuroprotection, wherein a PFKFB3 inhibitor is 2-{[8-(1-methyl-1H-indol-6-yl)quinoxalin-6-yl]amino}-N-[(1-methyl-1H-pyrazol-4-yl)methyl]benzene-1-sulfonamide

1397. PFKFB3 inhibitor for use in neuroprotection, wherein a PFKFB3 inhibitor is 2-{[8-(1-methyl-1H-indol-6-yl)quinoxalin-6-yl]amino}-N-[(oxan-4-yl)methyl]benzene-1-sulfonamide

1398. PFKFB3 inhibitor for use in neuroprotection, wherein a PFKFB3 inhibitor is 2-{[8-(1-methyl-1H-indol-6-yl)quinoxalin-6-yl]amino}-N-(1-methylpyrrolidin-3-yl)benzene-11-sulfonamide

1399. PFKFB3 inhibitor for use in neuroprotection, wherein a PFKFB3 inhibitor is N-(4-methanesulfonylpyridin-3-yl)-8-(1-methyl-1H-1,2,3-benzotriazol-6-yl)quinoxalin-6-amine

1400. PFKFB3 inhibitor for use in neuroprotection, wherein a PFKFB3 inhibitor is N-(4-methanesulfonylpyridin-3-yl)-8-(2-methyl-1,3-benzothiazol-5-yl)quinoxalin-6-amine

1401. PFKFB3 inhibitor for use in neuroprotection, wherein a PFKFB3 inhibitor is 2-{[8-(1-methyl-1H-indol-6-yl)quinoxalin-6-yl]amino}-N-[(1-methylpyrrolidin-3-yl)methyl]benzene-1-sulfonamide

1402. PFKFB3 inhibitor for use in neuroprotection, wherein a PFKFB3 inhibitor is N-(4-methanesulfonylpyridin-3-yl)-8-(1-methyl-1H-1,2,3-benzotriazol-5-yl)quinoxalin-6-amine

1403. PFKFB3 inhibitor for use in neuroprotection, wherein a PFKFB3 inhibitor is 8-(1-ethyl-1H-indol-6-yl)-N-(4-methanesulfonylpyridin-3-yl)quinoxalin-6-amine

1404. PFKFB3 inhibitor for use in neuroprotection, wherein a PFKFB3 inhibitor is N-(3-{7-[(4-methanesulfonylpyridin-3-yl)amino]quinoxalin-5-yl}phenyl)pyrrolidine-2-carboxamide

1405. PFKFB3 inhibitor for use in neuroprotection, wherein a PFKFB3 inhibitor is N-(4-{7-[(4-methanesulfonylpyridin-3-yl)amino]quinoxalin-5-yl}phenyl)pyrrolidine-2-carboxamide

1406. PFKFB3 inhibitor for use in neuroprotection, wherein a PFKFB3 inhibitor is N-(4-methanesulfonylpyridin-3-yl)-8-[3-(trifluoromethoxy)phenyl]quinoxalin-6-amine

1407. PFKFB3 inhibitor for use in neuroprotection, wherein a PFKFB3 inhibitor is 8-(2-amino-1,3-benzothiazol-5-yl)-N-(4-methanesulfonylpyridin-3-yl)quinoxalin-6-amine

1408. PFKFB3 inhibitor for use in neuroprotection, wherein a PFKFB3 inhibitor is 8-(1,2-benzothiazol-5-yl)-N-(4-methanesulfonylpyridin-3-yl)quinoxalin-6-amine

1409. PFKFB3 inhibitor for use in neuroprotection, wherein a PFKFB3 inhibitor is 2-(2-aminopyrimidin-4-yl)-3-{[8-(1-methyl-1H-indol-6-yl)quinoxalin-6-yl]amino}pyridine-4-carboxylic acid

1410. PFKFB3 inhibitor for use in neuroprotection, wherein a PFKFB3 inhibitor is 8-(3-bromophenyl)-N-(4-methanesulfonylpyridin-3-yl)quinoxalin-6-amine

1411. PFKFB3 inhibitor for use in neuroprotection, wherein a PFKFB3 inhibitor is 8-(4-bromophenyl)-N-(4-methanesulfonylpyridin-3-yl)quinoxalin-6-amine

1412. PFKFB3 inhibitor for use in neuroprotection, wherein a PFKFB3 inhibitor is 8-[1-(difluoromethyl)-1H-indol-6-yl]-N-(4-methanesulfonylpyridin-3-yl)quinoxalin-6-amine

1413. PFKFB3 inhibitor for use in neuroprotection, wherein a PFKFB3 inhibitor is N-(4-methanesulfonylpyridin-3-yl)-8-(5-methoxy-2-methylphenyl)quinoxalin-6-amine

1414. PFKFB3 inhibitor for use in neuroprotection, wherein a PFKFB3 inhibitor is N-(4-methanesulfonylpyridin-3-yl)-8-(4-methoxyphenyl)quinoxalin-6-amine

1415. PFKFB3 inhibitor for use in neuroprotection, wherein a PFKFB3 inhibitor is 3-{[8-(3-methyl-1-benzothiophen-5-yl)quinoxalin-6-yl]amino}-N-(1-methylpyrrolidin-3-yl)pyridine-4-carboxamide

1416. PFKFB3 inhibitor for use in neuroprotection, wherein a PFKFB3 inhibitor is 3-{[8-(3-methyl-1-benzothiophen-5-yl)quinoxalin-6-yl]amino}pyridine-4-carbonitrile

1417. PFKFB3 inhibitor for use in neuroprotection, wherein a PFKFB3 inhibitor is 5-{[8-(1-methyl-1H-indol-6-yl)quinoxalin-6-yl]amino}pyrimidine-4-carboxamide

1418. PFKFB3 inhibitor for use in neuroprotection, wherein a PFKFB3 inhibitor is N-(1-acetylpiperidin-3-yl)-3-{[8-(1-methyl-1H-indol-6-yl)quinoxalin-6-yl]amino}pyridine-4-carboxamide

1419. PFKFB3 inhibitor for use in neuroprotection, wherein a PFKFB3 inhibitor is N-(1-acetylpiperidin-4-yl)-3-{[8-(1-methyl-1H-indol-6-yl)quinoxalin-6-yl]amino}pyridine-4-carboxamide

1420. PFKFB3 inhibitor for use in neuroprotection, wherein a PFKFB3 inhibitor is 4-[(8-chloroquinoxalin-6-yl)amino]pyridine-3-carbonitrile

1421. PFKFB3 inhibitor for use in neuroprotection, wherein a PFKFB3 inhibitor is 4-{[8-(1-methyl-1H-indol-6-yl)quinoxalin-6-yl]amino}pyridine-3-carbonitrile

1422. PFKFB3 inhibitor for use in neuroprotection, wherein a PFKFB3 inhibitor is 3-{[8-(1-methyl-1H-indol-6-yl)quinoxalin-6-yl]amino}-N-[(pyridazin-3-yl)methyl]pyridine-4-carboxamide

1423. PFKFB3 inhibitor for use in neuroprotection, wherein a PFKFB3 inhibitor is N-[(1-methyl-1H-imidazol-5-yl)methyl]-3-{[8-(1-methyl-1H-indol-6-yl)quinoxalin-6-yl]amino}pyridine-4-carboxamide

1424. PFKFB3 inhibitor for use in neuroprotection, wherein a PFKFB3 inhibitor is 3-{[8-(1-methyl-1H-indol-6-yl)quinoxalin-6-yl]amino}-N-[(1-methylpyrrolidin-3-yl)methyl]pyridine-4-carboxamide

1425. PFKFB3 inhibitor for use in neuroprotection, wherein a PFKFB3 inhibitor is N-[(4-acetylmorpholin-2-yl)methyl]-3-{[8-(1-methyl-1H-indol-6-yl)quinoxalin-6-yl]amino}pyridine-4-carboxamide

1426. PFKFB3 inhibitor for use in neuroprotection, wherein a PFKFB3 inhibitor is N-[(1-acetylazetidin-3-yl)methyl]-3-{[8-(1-methyl-1H-indol-6-yl)quinoxalin-6-yl]amino}pyridine-4-carboxamide

1427. PFKFB3 inhibitor for use in neuroprotection, wherein a PFKFB3 inhibitor is 3-{[8-(1-methyl-1H-indol-6-yl)quinoxalin-6-yl]amino}-N-[(4-methylmorpholin-2-yl)methyl]pyridine-4-carboxamide

1428. PFKFB3 inhibitor for use in neuroprotection, wherein a PFKFB3 inhibitor is N-[(4-acetylmorpholin-3-yl)methyl]-3-{[8-(1-methyl-1H-indol-6-yl)quinoxalin-6-yl]amino}pyridine-4-carboxamide

1429. PFKFB3 inhibitor for use in neuroprotection, wherein a PFKFB3 inhibitor is 3-{[8-(1-methyl-1H-indol-6-yl)quinoxalin-6-yl]amino}-N-[(morpholin-3-yl)methyl]pyridine-4-carboxamide

1430. PFKFB3 inhibitor for use in neuroprotection, wherein a PFKFB3 inhibitor is 8-(3-aminophenyl)-N-(4-methanesulfonylpyridin-3-yl)quinoxalin-6-amine

1431. PFKFB3 inhibitor for use in neuroprotection, wherein a PFKFB3 inhibitor is 8-(4-aminophenyl)-N-(4-methanesulfonylpyridin-3-yl)quinoxalin-6-amine

1432. PFKFB3 inhibitor for use in neuroprotection, wherein a PFKFB3 inhibitor is N-(4-methanesulfonylpyridin-3-yl)-8-[3-(propan-2-yloxy)phenyl]quinoxalin-6-amine

1433. PFKFB3 inhibitor for use in neuroprotection, wherein a PFKFB3 inhibitor is 8-(3-ethoxyphenyl)-N-(4-methanesulfonylpyridin-3-yl)quinoxalin-6-amine

1434. PFKFB3 inhibitor for use in neuroprotection, wherein a PFKFB3 inhibitor is 2-{7-[(4-methanesulfonylpyridin-3-yl)amino]quinoxalin-5-yl}-4-methylbenzamide

1435. PFKFB3 inhibitor for use in neuroprotection, wherein a PFKFB3 inhibitor is 3-{[8-(1-methyl-1H-indol-6-yl)quinoxalin-6-yl]amino}-N-(2-oxopiperidin-4-yl)pyridine-4-carboxamide

1436. PFKFB3 inhibitor for use in neuroprotection, wherein a PFKFB3 inhibitor is N-cyclohexyl-3-{[8-(1-methyl-1H-indol-6-yl)quinoxalin-6-yl]amino}pyridine-4-carboxamide

1437. PFKFB3 inhibitor for use in neuroprotection, wherein a PFKFB3 inhibitor is 3-{methyl[8-(1-methyl-1H-indol-6-yl)quinoxalin-6-yl]amino}-N-(pyrimidin-5-yl)pyridine-4-carboxamide

1438. PFKFB3 inhibitor for use in neuroprotection, wherein a PFKFB3 inhibitor is 3-{[8-(1-methyl-1H-indol-6-yl)quinoxalin-6-yl]amino}-N-(1-methylpiperidin-3-yl)pyridine-4-carboxamide

1439. PFKFB3 inhibitor for use in neuroprotection, wherein a PFKFB3 inhibitor is 3-{[8-(1-methyl-1H-indol-6-yl)quinoxalin-6-yl]amino}-N-(1-methylpiperidin-4-yl)pyridine-4-carboxamide

1440. PFKFB3 inhibitor for use in neuroprotection, wherein a PFKFB3 inhibitor is 3-{[8-(1-methyl-1H-indol-6-yl)quinoxalin-6-yl]amino}-N-(1-methyl-6-oxopiperidin-3-yl)pyridine-4-carboxamide

1441. PFKFB3 inhibitor for use in neuroprotection, wherein a PFKFB3 inhibitor is N-(1-acetylpyrrolidin-3-yl)-3-{[8-(1-methyl-1H-indol-6-yl)quinoxalin-6-yl]amino}pyridine-4-carboxamide

1442. PFKFB3 inhibitor for use in neuroprotection, wherein a PFKFB3 inhibitor is 8-(2,1,3-benzoxadiazol-5-yl)-N-(4-methanesulfonylpyridin-3-yl)quinoxalin-6-amine

1443. PFKFB3 inhibitor for use in neuroprotection, wherein a PFKFB3 inhibitor is 4-methanesulfonyl-3-{[8-(1-methyl-1H-indol-6-yl)quinoxalin-6-yl]amino}benzohydrazide

1444. PFKFB3 inhibitor for use in neuroprotection, wherein a PFKFB3 inhibitor is 8-(2H-1,2,3-benzotriazol-5-yl)-N-(4-methanesulfonylpyridin-3-yl)quinoxalin-6-amine

1445. PFKFB3 inhibitor for use in neuroprotection, wherein a PFKFB3 inhibitor is 8-(2,1,3-benzothiadiazol-5-yl)-N-(4-methanesulfonylpyridin-3-yl)quinoxalin-6-amine

1446. PFKFB3 inhibitor for use in neuroprotection, wherein a PFKFB3 inhibitor is 4-methanesulfonyl-3-{[8-(1-methyl-1H-indol-6-yl)quinoxalin-6-yl]amino}benzamide

1447. PFKFB3 inhibitor for use in neuroprotection, wherein a PFKFB3 inhibitor is methyl 4-methanesulfonyl-3-{[8-(1-methyl-1H-indol-6-yl)quinoxalin-6-yl]amino}benzoate

1448. PFKFB3 inhibitor for use in neuroprotection, wherein a PFKFB3 inhibitor is N-(1-methyl-1H-1,2,3-triazol-5-yl)-8-(1-methyl-1H-indol-6-yl)quinoxalin-6-amine

1449. PFKFB3 inhibitor for use in neuroprotection, wherein a PFKFB3 inhibitor is N-(4-methanesulfonylpyridin-3-yl)-8-(3-methyl-1-benzothiophen-5-yl)quinoxalin-6-amine

1450. PFKFB3 inhibitor for use in neuroprotection, wherein a PFKFB3 inhibitor is N-(4-methanesulfonylpyridin-3-yl)-N-methyl-8-(1-methyl-1H-indol-6-yl)quinoxalin-6-amine

1451. PFKFB3 inhibitor for use in neuroprotection, wherein a PFKFB3 inhibitor is N-(2-methanesulfonyl-5-nitrophenyl)-8-(3-methyl-1-benzofuran-5-yl)quinoxalin-6-amine

1452. PFKFB3 inhibitor for use in neuroprotection, wherein a PFKFB3 inhibitor is 6-methanesulfonyl-N1-[8-(3-methyl-1-benzofuran-5-yl)quinoxalin-6-yl]benzene-1,3-diamine

1453. PFKFB3 inhibitor for use in neuroprotection, wherein a PFKFB3 inhibitor is 3-{[8-(1-methyl-1H-indol-6-yl)quinoxalin-6-yl]amino}-N-(oxan-4-yl)pyridine-4-carboxamide

1454. PFKFB3 inhibitor for use in neuroprotection, wherein a PFKFB3 inhibitor is 2-{[8-(1-methyl-1H-indol-6-yl)quinoxalin-6-yl]amino}-N-(pyrimidin-5-yl)benzene-1-sulfonamide

1455. PFKFB3 inhibitor for use in neuroprotection, wherein a PFKFB3 inhibitor is 3-{[8-(1-methyl-1H-indol-6-yl)quinoxalin-6-yl]amino}-N-(1-methylpyrrolidin-3-yl)pyridine-4-carboxamide

1456. PFKFB3 inhibitor for use in neuroprotection, wherein a PFKFB3 inhibitor is N-(1-acetylazetidin-3-yl)-3-{[8-(1-methyl-1H-indol-6-yl)quinoxalin-6-yl]amino}pyridine-4-carboxamide

1457. PFKFB3 inhibitor for use in neuroprotection, wherein a PFKFB3 inhibitor is 3-{[8-(1-methyl-1H-indol-6-yl)quinoxalin-6-yl]amino}-N-(1-methyl-2-oxopiperidin-4-yl)pyridine-4-carboxamide

1458. PFKFB3 inhibitor for use in neuroprotection, wherein a PFKFB3 inhibitor is 3-{[8-(1-methyl-1H-indol-6-yl)quinoxalin-6-yl]amino}-N-phenylpyridine-4-carboxamide

1459. PFKFB3 inhibitor for use in neuroprotection, wherein a PFKFB3 inhibitor is 3-{methyl[8-(1-methyl-1H-indol-6-yl)quinoxalin-6-yl]amino}pyridine-4-carboxamide

1460. PFKFB3 inhibitor for use in neuroprotection, wherein a PFKFB3 inhibitor is N-[2-methanesulfonyl-5-(1,3-oxazol-2-yl)phenyl]-8-(1-methyl-1H-indol-6-yl)quinoxalin-6-amine

1461. PFKFB3 inhibitor for use in neuroprotection, wherein a PFKFB3 inhibitor is N-[2-methanesulfonyl-5-(1-methyl-1H-pyrazol-5-yl)phenyl]-8-(1-methyl-1H-indol-6-yl)quinoxalin-6-amine

1462. PFKFB3 inhibitor for use in neuroprotection, wherein a PFKFB3 inhibitor is 3-{[8-(1-methyl-1H-indol-6-yl)quinoxalin-6-yl]amino}-N-(1-methyl-1H-pyrazol-4-yl)pyridine-4-carboxamide

1463. PFKFB3 inhibitor for use in neuroprotection, wherein a PFKFB3 inhibitor is 3-{methyl[8-(1-methyl-1H-indol-6-yl)quinoxalin-6-yl]amino}pyridine-4-carbonitrile

1464. PFKFB3 inhibitor for use in neuroprotection, wherein a PFKFB3 inhibitor is 4-cyano-3-{[8-(1-methyl-1H-indol-6-yl)quinoxalin-6-yl]amino}pyridin-1-ium-1-olate

1465. PFKFB3 inhibitor for use in neuroprotection, wherein a PFKFB3 inhibitor is 2-{[8-(1-methyl-1H-indol-6-yl)quinoxalin-6-yl]amino}benzamide

1466. PFKFB3 inhibitor for use in neuroprotection, wherein a PFKFB3 inhibitor is 2-{[8-(1-methyl-1H-indol-6-yl)quinoxalin-6-yl]amino}benzonitrile

1467. PFKFB3 inhibitor for use in neuroprotection, wherein a PFKFB3 inhibitor is 2-{[8-(1-methyl-1H-indol-6-yl)quinoxalin-6-yl]amino}-N-[(pyrimidin-5-yl)methyl]benzene-1-sulfonamide

1468. PFKFB3 inhibitor for use in neuroprotection, wherein a PFKFB3 inhibitor is 8-(1-methyl-1H-indol-6-yl)-N-{2H,3H,4H-pyrido[4,3-b][1,4]oxazin-8-yl}quinoxalin-6-amine

1469. PFKFB3 inhibitor for use in neuroprotection, wherein a PFKFB3 inhibitor is N-[4-(1-methyl-1H-imidazol-4-yl)pyridin-3-yl]-8-(1-methyl-1H-indol-6-yl)quinoxalin-6-amine

1470. PFKFB3 inhibitor for use in neuroprotection, wherein a PFKFB3 inhibitor is 1-[4-(3-{[8-(1-methyl-1H-indol-6-yl)quinoxalin-6-yl]amino}pyridin-4-yl)piperazin-1-yl]ethan-1-one

1471. PFKFB3 inhibitor for use in neuroprotection, wherein a PFKFB3 inhibitor is N-(3-methanesulfonylpyridin-2-yl)-8-(1-methyl-1H-indol-6-yl)quinoxalin-6-amine

1472. PFKFB3 inhibitor for use in neuroprotection, wherein a PFKFB3 inhibitor is 8-[2-(dimethylamino)-5-methylphenyl]-N-(4-methanesulfonylpyridin-3-yl)quinoxalin-6-amine

1473. PFKFB3 inhibitor for use in neuroprotection, wherein a PFKFB3 inhibitor is 8-(3-amino-4-methylphenyl)-N-(4-methanesulfonylpyridin-3-yl)quinoxalin-6-amine

1474. PFKFB3 inhibitor for use in neuroprotection, wherein a PFKFB3 inhibitor is N-(4-methanesulfonylpyridin-3-yl)-8-(2-methoxy-5-methylphenyl)quinoxalin-6-amine

1475. PFKFB3 inhibitor for use in neuroprotection, wherein a PFKFB3 inhibitor is 8-(5-fluoro-1-methyl-1H-indol-6-yl)-N-(4-methanesulfonylpyridin-3-yl)quinoxalin-6-amine

1476. PFKFB3 inhibitor for use in neuroprotection, wherein a PFKFB3 inhibitor is 4-methanesulfonyl-3-{[8-(3-methyl-1-benzofuran-5-yl)quinoxalin-6-yl]amino}pyridin-1-ium-1-olate

1477. PFKFB3 inhibitor for use in neuroprotection, wherein a PFKFB3 inhibitor is 8-(4-fluoro-1-methyl-1H-indol-6-yl)-N-(4-methanesulfonylpyridin-3-yl)quinoxalin-6-amine

1478. PFKFB3 inhibitor for use in neuroprotection, wherein a PFKFB3 inhibitor is N-(4-chloropyridin-3-yl)-8-(1-methyl-1H-indol-6-yl)quinoxalin-6-amine

1479. PFKFB3 inhibitor for use in neuroprotection, wherein a PFKFB3 inhibitor is 8-(1-methyl-1H-indol-6-yl)-N-[4-(2H-1,2,3,4-tetrazol-5-yl)pyridin-3-yl]quinoxalin-6-amine

1480. PFKFB3 inhibitor for use in neuroprotection, wherein a PFKFB3 inhibitor is 2-{7-[(4-methanesulfonylpyridin-3-yl)amino]quinoxalin-5-yl}-4-methylphenol

1481. PFKFB3 inhibitor for use in neuroprotection, wherein a PFKFB3 inhibitor is 8-(2-amino-5-methylphenyl)-N-(4-methanesulfonylpyridin-3-yl)quinoxalin-6-amine

1482. PFKFB3 inhibitor for use in neuroprotection, wherein a PFKFB3 inhibitor is 8-(3-ethylphenyl)-N-(4-methanesulfonylpyridin-3-yl)quinoxalin-6-amine

1483. PFKFB3 inhibitor for use in neuroprotection, wherein a PFKFB3 inhibitor is 8-(3,3-dimethyl-2,3-dihydro-1-benzofuran-5-yl)-N-(4-methanesulfonylpyridin-3-yl)quinoxalin-6-amine

1484. PFKFB3 inhibitor for use in neuroprotection, wherein a PFKFB3 inhibitor is N-(4-methanesulfonylpyridin-3-yl)-8-(3-methoxyphenyl)quinoxalin-6-amine

1485. PFKFB3 inhibitor for use in neuroprotection, wherein a PFKFB3 inhibitor is 8-(1H-1,3-benzodiazol-6-yl)-N-(4-methanesulfonylpyridin-3-yl)quinoxalin-6-amine

1486. PFKFB3 inhibitor for use in neuroprotection, wherein a PFKFB3 inhibitor is 8-(4-ethylphenyl)-N-(4-methanesulfonylpyridin-3-yl)quinoxalin-6-amine

1487. PFKFB3 inhibitor for use in neuroprotection, wherein a PFKFB3 inhibitor is 8-(7-fluoro-1-methyl-1H-indol-6-yl)-N-(4-methanesulfonylpyridin-3-yl)quinoxalin-6-amine

1488. PFKFB3 inhibitor for use in neuroprotection, wherein a PFKFB3 inhibitor is 8-[3-(chloromethyl)-1-benzofuran-5-yl]-N-(4-methanesulfonylpyridin-3-yl)quinoxalin-6-amine

1489. PFKFB3 inhibitor for use in neuroprotection, wherein a PFKFB3 inhibitor is 4-methanesulfonyl-N1-methyl-N3-[8-(1-methyl-1H-indol-6-yl)quinoxalin-6-yl]benzene-1,3-diamine

1490. PFKFB3 inhibitor for use in neuroprotection, wherein a PFKFB3 inhibitor is 3-{[8-(1-methyl-1H-indol-6-yl)quinoxalin-6-yl]amino}-N-[(1-methyl-1H-pyrazol-4-yl)methyl]pyridine-4-carboxamide

1491. PFKFB3 inhibitor for use in neuroprotection, wherein a PFKFB3 inhibitor is 3-{[8-(1-methyl-1H-indol-6-yl)quinoxalin-6-yl]amino}-N-[(pyrimidin-5-yl)methyl]pyridine-4-carboxamide

1492. PFKFB3 inhibitor for use in neuroprotection, wherein a PFKFB3 inhibitor is 3-{[8-(1-methyl-1H-indol-6-yl)quinoxalin-6-yl]amino}-N-(pyrimidin-5-yl)pyridine-4-carboxamide

1493. PFKFB3 inhibitor for use in neuroprotection, wherein a PFKFB3 inhibitor is N,N-dimethyl-3-{[8-(1-methyl-1H-indol-6-yl)quinoxalin-6-yl]amino}pyridine-4-carboxamide

1494. PFKFB3 inhibitor for use in neuroprotection, wherein a PFKFB3 inhibitor is N-methyl-3-{[8-(1-methyl-1H-indol-6-yl)quinoxalin-6-yl]amino}pyridine-4-carboxamide

1495. PFKFB3 inhibitor for use in neuroprotection, wherein a PFKFB3 inhibitor is N-(4-methanesulfonylpyridin-3-yl)-8-(3-methylphenyl)quinoxalin-6-amine

1496. PFKFB3 inhibitor for use in neuroprotection, wherein a PFKFB3 inhibitor is 8-[3-(dimethylamino)phenyl]-N-(4-methanesulfonylpyridin-3-yl)quinoxalin-6-amine

1497. PFKFB3 inhibitor for use in neuroprotection, wherein a PFKFB3 inhibitor is N-(4-methanesulfonylpyridin-3-yl)-8-[1-(propan-2-yl)-1H-indol-6-yl]quinoxalin-6-amine

1498. PFKFB3 inhibitor for use in neuroprotection, wherein a PFKFB3 inhibitor is N-(4-methanesulfonylpyridin-3-yl)-8-[3-(2H-1,2,3-triazol-4-yl)phenyl]quinoxalin-6-amine

1499. PFKFB3 inhibitor for use in neuroprotection, wherein a PFKFB3 inhibitor is 3-{[8-(1-methyl-1H-indol-6-yl)quinoxalin-6-yl]oxy}pyridine-4-carbonitrile

1500. PFKFB3 inhibitor for use in neuroprotection, wherein a PFKFB3 inhibitor is 1-[4-(4-methanesulfonyl-3-{[8-(1-methyl-1H-indol-6-yl)quinoxalin-6-yl]amino}phenyl)piperazin-1-yl]ethan-1-one

1501. PFKFB3 inhibitor for use in neuroprotection, wherein a PFKFB3 inhibitor is N-[2-methanesulfonyl-5-(4-methylpiperazin-1-yl)phenyl]-8-(1-methyl-1H-indol-6-yl)quinoxalin-6-amine

1502. PFKFB3 inhibitor for use in neuroprotection, wherein a PFKFB3 inhibitor is 4-methanesulfonyl-3-{[8-(1-methyl-1H-indol-6-yl)quinoxalin-6-yl]amino}pyridin-1-ium-1-olate

1503. PFKFB3 inhibitor for use in neuroprotection, wherein a PFKFB3 inhibitor is N-[2-methanesulfonyl-5-(2H-1,2,3,4-tetrazol-5-yl)phenyl]-8-(1-methyl-1H-indol-6-yl)quinoxalin-6-amine

1504. PFKFB3 inhibitor for use in neuroprotection, wherein a PFKFB3 inhibitor is N-[5-(1H-imidazol-1-yl)-2-methanesulfonylphenyl]-8-(1-methyl-1H-indol-6-yl)quinoxalin-6-amine

1505. PFKFB3 inhibitor for use in neuroprotection, wherein a PFKFB3 inhibitor is N-(4-methanesulfonyl-3-{[8-(1-methyl-1H-indol-6-yl)quinoxalin-6-yl]amino}phenyl)acetamide

1506. PFKFB3 inhibitor for use in neuroprotection, wherein a PFKFB3 inhibitor is 8-(1-methyl-1H-indol-6-yl)-N-[4-(4-methylpiperazin-1-yl)pyridin-3-yl]quinoxalin-6-amine

1507. PFKFB3 inhibitor for use in neuroprotection, wherein a PFKFB3 inhibitor is 8-(2,5-dimethylphenyl)-N-(4-methanesulfonylpyridin-3-yl)quinoxalin-6-amine

1508. PFKFB3 inhibitor for use in neuroprotection, wherein a PFKFB3 inhibitor is N-[5-(aminomethyl)-2-methanesulfonylphenyl]-8-(1-methyl-1H-indol-5-yl)quinoxalin-6-amine

1509. PFKFB3 inhibitor for use in neuroprotection, wherein a PFKFB3 inhibitor is 8-(2,3-dihydro-1-benzofuran-5-yl)-N-(4-methanesulfonylpyridin-3-yl)quinoxalin-6-amine

1510. PFKFB3 inhibitor for use in neuroprotection, wherein a PFKFB3 inhibitor is 6-methanesulfonyl-N1-[8-(1-methyl-1H-indol-6-yl)quinoxalin-6-yl]benzene-1,3-diamine

1511. PFKFB3 inhibitor for use in neuroprotection, wherein a PFKFB3 inhibitor is N-(2-methanesulfonyl-5-nitrophenyl)-8-(1-methyl-1H-indol-6-yl)quinoxalin-6-amine

1512. PFKFB3 inhibitor for use in neuroprotection, wherein a PFKFB3 inhibitor is 5-(1-methyl-1H-indol-5-yl)-7-{1H,2H,3H-pyrrolo[2,3-c]pyridin-1-yl}quinoxaline

1513. PFKFB3 inhibitor for use in neuroprotection, wherein a PFKFB3 inhibitor is 8-(1-methyl-1H-indol-5-yl)-N-[4-(pyrimidin-5-yl)pyridin-3-yl]quinoxalin-6-amine

1514. PFKFB3 inhibitor for use in neuroprotection, wherein a PFKFB3 inhibitor is 8-(1-methyl-1H-indol-5-yl)-N-[4-(4-methylpiperazin-1-yl)pyridin-3-yl]quinoxalin-6-amine

1515. PFKFB3 inhibitor for use in neuroprotection, wherein a PFKFB3 inhibitor is 8-(1-methyl-1H-indol-5-yl)-N-[4-(1-methyl-1H-pyrazol-4-yl)pyridin-3-yl]quinoxalin-6-amine

1516. PFKFB3 inhibitor for use in neuroprotection, wherein a PFKFB3 inhibitor is N-(4-chloropyridin-3-yl)-8-(1-methyl-1H-indol-5-yl)quinoxalin-6-amine

1517. PFKFB3 inhibitor for use in neuroprotection, wherein a PFKFB3 inhibitor is 3-{[8-(1-methyl-1H-indol-5-yl)quinoxalin-6-yl]amino}pyridine-4-carboxamide

1518. PFKFB3 inhibitor for use in neuroprotection, wherein a PFKFB3 inhibitor is 3-{[8-(1-methyl-1H-indol-5-yl)quinoxalin-6-yl]amino}pyridine-4-carbonitrile

1519. PFKFB3 inhibitor for use in neuroprotection, wherein a PFKFB3 inhibitor is N-(5-methanesulfonylpyrimidin-4-yl)-8-(1-methyl-1H-indol-6-yl)quinoxalin-6-amine

1520. PFKFB3 inhibitor for use in neuroprotection, wherein a PFKFB3 inhibitor is 3-{[8-(3-methyl-1-benzofuran-5-yl)quinoxalin-6-yl]amino}pyridine-4-carboxamide

1521. PFKFB3 inhibitor for use in neuroprotection, wherein a PFKFB3 inhibitor is 4-methanesulfonyl-3-{[8-(1-methyl-1H-indol-6-yl)quinoxalin-6-yl]amino}benzonitrile

1522. PFKFB3 inhibitor for use in neuroprotection, wherein a PFKFB3 inhibitor is 3-{[8-(3-methyl-1-benzofuran-5-yl)quinoxalin-6-yl]amino}pyridine-4-carbonitrile

1523. PFKFB3 inhibitor for use in neuroprotection, wherein a PFKFB3 inhibitor is N-(4-methoxypyridin-3-yl)-8-(1-methyl-1H-indol-6-yl)quinoxalin-6-amine

1524. PFKFB3 inhibitor for use in neuroprotection, wherein a PFKFB3 inhibitor is N-(4-methanesulfonylpyridin-3-yl)-8-(3-methyl-1-benzofuran-5-yl)quinoxalin-6-amine

1525. PFKFB3 inhibitor for use in neuroprotection, wherein a PFKFB3 inhibitor is N-(2-methanesulfonylphenyl)-8-{1-methyl-1H-pyrrolo[2,3-b]pyridin-6-yl}quinoxalin-6-amine

1526. PFKFB3 inhibitor for use in neuroprotection, wherein a PFKFB3 inhibitor is N,N-dimethyl-2-{[8-(1-methyl-1H-indol-6-yl)quinoxalin-6-yl]amino}benzene-1-sulfonamide

1527. PFKFB3 inhibitor for use in neuroprotection, wherein a PFKFB3 inhibitor is 3-{[8-(1-methyl-1H-indol-6-yl)quinoxalin-6-yl]amino}pyridine-4-carboxamide

1528. PFKFB3 inhibitor for use in neuroprotection, wherein a PFKFB3 inhibitor is 3-{[8-(1-methyl-1H-indol-6-yl)quinoxalin-6-yl]amino}pyridine-4-carbonitrile

1529. PFKFB3 inhibitor for use in neuroprotection, wherein a PFKFB3 inhibitor is N3-[8-(1-methyl-1H-indol-6-yl)quinoxalin-6-yl]pyridine-2,3-diamine

1530. PFKFB3 inhibitor for use in neuroprotection, wherein a PFKFB3 inhibitor is N-methyl-2-{[8-(1-methyl-1H-indol-6-yl)quinoxalin-6-yl]amino}benzene-1-sulfonamide

1531. PFKFB3 inhibitor for use in neuroprotection, wherein a PFKFB3 inhibitor is 8-(1-methyl-1H-indol-6-yl)-N-[2-(piperazine-1-sulfonyl)phenyl]quinoxalin-6-amine

1532. PFKFB3 inhibitor for use in neuroprotection, wherein a PFKFB3 inhibitor is 3-{[8-(1-methyl-1H-indol-6-yl)quinoxalin-6-yl]amino}-1,2-dihydropyridin-2-one

1533. PFKFB3 inhibitor for use in neuroprotection, wherein a PFKFB3 inhibitor is N-(2-methoxypyridin-3-yl)-8-(1-methyl-1H-indol-6-yl)quinoxalin-6-amine

1534. PFKFB3 inhibitor for use in neuroprotection, wherein a PFKFB3 inhibitor is N-(4-methanesulfonylpyridin-3-yl)-8-(1-methyl-1H-indol-6-yl)quinoxalin-6-amine

1535. PFKFB3 inhibitor for use in neuroprotection, wherein a PFKFB3 inhibitor is N-(5-bromo-2-methanesulfonylphenyl)-8-(1-methyl-1H-indol-6-yl)quinoxalin-6-amine

1536. PFKFB3 inhibitor for use in neuroprotection, wherein a PFKFB3 inhibitor is 8-(1,3-benzothiazol-5-yl)-N-(4-methanesulfonylpyridin-3-yl)quinoxalin-6-amine

1537. PFKFB3 inhibitor for use in neuroprotection, wherein a PFKFB3 inhibitor is 2-{[8-(1-methyl-1H-indol-6-yl)quinoxalin-6-yl]amino}benzene-1-sulfonamide

1538. PFKFB3 inhibitor for use in neuroprotection, wherein a PFKFB3 inhibitor is 8-(1-methyl-1H-indol-6-yl)-N-[2-(morpholine-4-sulfonyl)phenyl]quinoxalin-6-amine

1539. PFKFB3 inhibitor for use in neuroprotection, wherein a PFKFB3 inhibitor is N-(2-methanesulfonylpyridin-3-yl)-8-(1-methyl-1H-indol-6-yl)quinoxalin-6-amine

1540. PFKFB3 inhibitor for use in neuroprotection, wherein a PFKFB3 inhibitor is 8-(2-chloro-5-methoxyphenyl)-N-(4-methanesulfonylpyridin-3-yl)quinoxalin-6-amine

1541. PFKFB3 inhibitor for use in neuroprotection, wherein a PFKFB3 inhibitor is 8-(1,3-benzothiazol-6-yl)-N-(4-methanesulfonylpyridin-3-yl)quinoxalin-6-amine

1542. PFKFB3 inhibitor for use in neuroprotection, wherein a PFKFB3 inhibitor is N-(2-methanesulfonylphenyl)-8-(1-methyl-1H-indol-6-yl)quinoxalin-6-amine

1543. PFKFB3 inhibitor for use in neuroprotection, wherein a PFKFB3 inhibitor is 5-(1-methyl-1H-indol-6-yl)-7-{1H,2H,3H-pyrrolo[2,3-c]pyridin-1-yl}quinoxaline

1544. PFKFB3 inhibitor for use in neuroprotection, wherein a PFKFB3 inhibitor is 8-(2,3-dihydro-1,4-benzodioxin-6-yl)-N-(4-methanesulfonylpyridin-3-yl)quinoxalin-6-amine

Accordingly, the present invention also relates to the following A-H items:
since the compounds are known in the art every Item A-G contain the reference to the informational source (patent application or publication incorporated here by reference), in case of any errors or ambiguity in chemical names or structures etc. in present application in the said A-G items the information from referenced relative patent application (such as WO2012035171A3, WO2011161201A1, WO2012149528A1, WO2016180537A1, WO2018087021A1 etc) or publication should be used as an original source of correct information on chemical structures of PFKFB3 inhibitors.
Every of the following “letter” items A-H, can comprise several further “numbered” items, which are crossferenced inside same “letter” item. It is also references to all items of this application when referencing to it as “in any one of preceding items”.

The reference to numbered formulas in items inside items A-H relates to the formula having a number in corresponding item A-H, e.g. item 8 of item C comprises wording “A compound of formula (I) wherein: n is 0 or 1 . . . ”, the formula (I) defined in this item C is meant.

Item A

(AstraZeneca https://dx.doi.org/10.1021/acs.jmedchem.5b00352)

PFKFB3 inhibitor for use in neuroprotection, wherein PFKFB3 inhibitor is selected from those described in https://dx.doi.org/10.1021/acs.imedchem.5b00352 incorporated here by reference.

PFKFB3 inhibitor for use in neuroprotection, wherein PFKFB3 inhibitor is a compound of Formula (I):

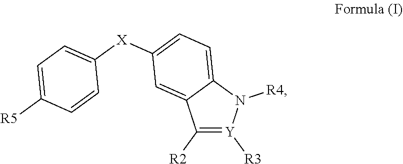

or a pharmaceutically acceptable salt thereof, wherein:

each X is independently selected from —O—, —S—, —NR⁷— or —CR⁷R⁸—;

each Y is independently selected from C or N;

each R⁷ and R⁸ is independently selected from hydrogen and C₁-C₆ alkyl, C₁-C₆alkoxy, wherein said alkyl is optionally substituted with one or more halogens;

R² is selected from hydrogen, halogens, nitrile and

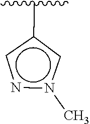

R³ is selected from hydrogen and —NR⁷R⁸

R⁴ is selected from hydrogen, ₁-C₆ alkyl, C₁-C₆alkoxy, aryl, heteroaryl, C₃-C₈ cycloalkyl, 10-membered heterocycloalkyl,

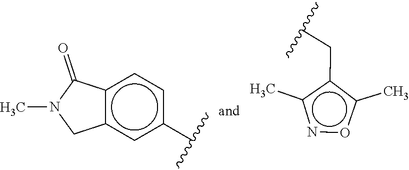

and wherein the C₁-C₆alkyl and C₁-C₆alkoxy are optionally substituted with one or more substituents independently selected at each occurrence from halogens, —C(═O)NR⁷R⁸ and R²; and

wherein heteroaryl, C₃-C₈ cycloalkyl, —O—C₃-C₈ cycloalkyl, 3- to 10-memberedheterocycloalkyl and —O—(3- to 10-memberedheterocycloalkyl) are optionally substituted with one or more R²;

R⁵ is selected from

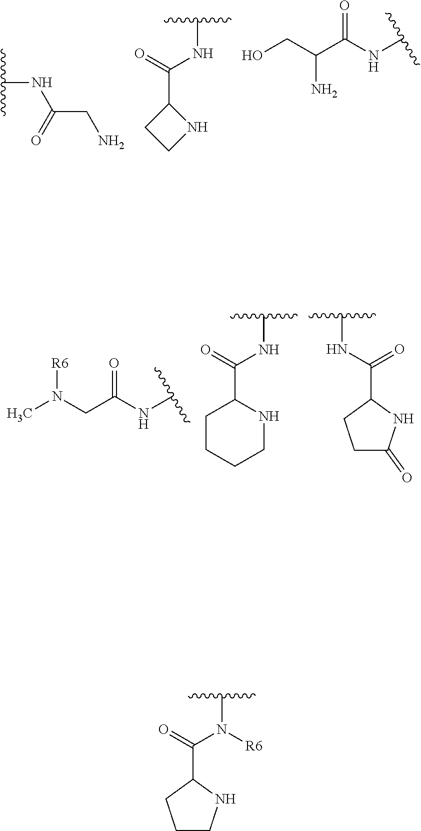

R⁶ is selected from hydrogen and ₁-C₆ alkyl.

Item B

PFKFB3 inhibitor for use in neuroprotection, wherein PFKFB3 is selected from those described in WO2012035171A3 incorporated here by reference.

PFKFB3 inhibitor for use in neuroprotection, wherein PFKFB3 inhibitor is selected from the following

1. A compound of formula (I)

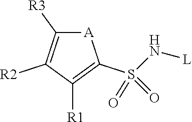

wherein: (i) A is O or S; and R¹ is selected from H; halogen; and C₁-C₆ alkyl optionally substituted with at least one halogen; or

R¹ and R² form, together with the carbon atoms to which they are attached, a benzene ring optionally substituted with at least one R⁶; or a 5- or 6-membered heteroaromatic or heterocyclic ring, optionally substituted with at least one R⁶;

R² and R³ are independently selected from H; halogen; C₁-C₆ alkyl optionally substituted with at least one halogen; phenyl optionally substituted with at least one R⁶; 5- or 6-membered heteroaryl optionally substituted with at least one R⁶; and 5- or 6-membered arylsulfonyl or heteroaryl sulfonyl, optionally substituted with at least one R⁶; provided that
at least one of R² and R³ is selected from said phenyl, heteroaryl, arylsulfonyl and heteroarylsulfonyl, and
when L is (a), neither R² nor R³ is unsubstituted phenyl; or

R² and R³ form, together with the carbon atoms to which they are attached, a benzene ring optionally substituted with at least one R⁶; or a 5- or 6-membered heteroaromatic or hetero-cyclic ring, optionally substituted with at least one R⁶; or

(ii) A is CR″ ═CR′;

each R′ is independently selected from H; halogen; and C1-C6 alkyl optionally substituted with at least one halogen;

R¹ is selected from H; halogen; and C1-C6 alkyl optionally substituted with at least one halogen; or

R¹ and R² form, together with the carbon atoms to which they are attached, a benzene ring optionally substituted with at least one R⁶; or a 5- or 6-membered heteroaromatic or heterocyclic ring, optionally substituted with at least one R⁶;

R² and R³ are independently selected from H, halogen, C1-C6 alkyl optionally substituted with at least one halogen; phenyl optionally substituted with at least one R⁶; 5- or 6-membered heteroaryl optionally substituted with at least one R⁶; and 5- or 6-membered aryl-sulfonyl or heteroaryl sulfonyl, optionally substituted with at least one R⁶; provided that:
when both R² and R³ are selected from H, halogen and C1-C6 alkyl optionally substituted with at least one halogen, the ring containing A is substituted in ortho position relative to the sulphonamide bond with at least one substituent selected from halogen and C1-C6 alkyl optionally substituted with at least one halogen;
when L is (a), neither R² nor R³ is unsubstituted phenyl; and
when L is (c), R³ is optionally substituted phenyl only when R⁵ is tetrazol-5-yl, and R² is unsubstituted phenyl only when R⁴ is not hydroxy; or

R² and R³ form, together with the carbon atoms to which they are attached, a benzene ring optionally substituted with at least one R⁶, provided that said benzene ring is unsubstituted only when R⁵ is tetrazolyl or oxazolyl; or a 5- or 6-membered heteroaromatic or heterocyclic ring, optionally substituted with at least one R⁶;

L is

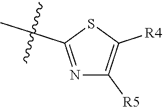

wherein R⁴ is COOR¹²; and R⁵ is selected from H and C1-C6 alkyl; or

R⁴ is selected from H and C1-C6 alkyl; and R⁵ is COOR¹²; or

(b)

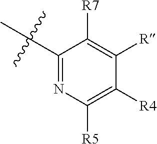

wherein R⁴ is selected from H and C1-C6 alkyl; R⁵ is selected from H and C1-C6 alkyl; and R″ is selected from C0-C1 alkyl-COOR¹²; or

R⁵ is selected from COOR¹²; and R″ is selected from H and C1-C6 alkyl; and

R is selected from H, C1-C6 alkyl, and nitro;

(c)

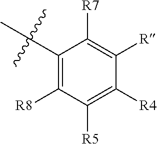

wherein R⁴ is selected from H, hydroxy and C1-C6 alkyl; R⁵ is selected from H, C1-C6 alkyl; and R″ is selected from C0-C1 alkyl-COOR¹²; or

R⁵ is selected from COOR¹², oxazol-5-yl and tetrazol-5-yl, said oxazol-5-yl and tetrazol-5-yl optionally being substituted by R⁹; and R″ is selected from H, C1-C6 alkyl, and nitro;

R⁷ is selected from H, C1-C6 alkyl, and nitro; and

R is selected from H, hydroxy, and C1-C6 alkyl; or

(d)

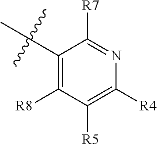

wherein R⁴ is selected from H and C1-C6 alkyl; and R⁵ is COOR¹²;

R⁷ is selected from H, C1-C6 alkyl, and nitro; and R⁸ is selected from H, hydroxy, and C1-C6 alkyl;

provided that in any of (a), (b), (c) and (d), R⁴, R⁵ and R″ are selected from C0-C1 alkyl-COOR¹² only when at least one of R² or R³ is optionally substituted phenyl or optionally substituted heteroaryl; or when R² and R³ together with the carbon atoms to which they are attached form a benzene ring optionally substituted by at least one R⁶;

R⁶ is selected from C1-C6 alkyl, cyano, halogen, hydroxy, C1-C6 alkoxy, C1-C6 alkylthio, tetrahydropyrrolyl, R¹⁰R¹¹N, carbamoyl, and C1-C6 alkylcarbonylamino, or is an ethyleneoxy biradical forming, together with the atoms to which it is attached, a five-membered oxygen containing cycle; wherein any alkyl is optionally substituted with at least one halogen;

R⁹ is selected from C0-C1 alkyl-COOR¹²;

R¹⁰ and R¹¹ are independently selected from H and C1-C6 alkyl or form, together with the nitrogen to which they are attached, a 5- or 6-membered cyclic amino optionally containing one other cyclic heteroatom;

R¹² is selected from H, C1-C6 alkyl; heteroaryl-C0-C2 alkyl; (C1-C3 alkoxy)ₚC1-C3 alkyl; aryl-C0-C2 alkyl; heterocyclyl-C0-C2 alkyl; and C1-C6 dialkylamino-C1-C6 alkyl, wherein any cyclic moiety is optionally substituted with C1-C6 alkyl; p is 1 or 2; or a pharmaceutically acceptable salt thereof;

provided that the compound is not:
ethyl 2-(benzofuran-2-sulfonamido)thiazole-4-carboxylate;
ethyl 2-(5-methylbenzo[b]thiophene-2-sulfonamido)thiazole-4-carboxylate;
ethyl 2-(benzo[b]thiophene-2-sulfonamido)thiazole-4-carboxylate;
ethyl 2-(6-acetamidonaphthalene-2-sulfonamido)-4-methylthiazole-5-carboxylate;
ethyl 2-(6-aminonaphthalene-2-sulfonamido)-4-methylthiazole-5-carboxylate;
methyl 6-(4′-cyano-[1,1′-biphenyl]-4-ylsulfonamido)picolinate;
2-(3-(benzo[b]thiophene-2-sulfonamido)phenyl)acetic acid;
methyl 2-(3-(benzo[b]thiophene-2-sulfonamido)phenyl)acetate;
ethyl 3-(5-(6-oxo-1,6-dihydropyridazin-3-yl)furan-2-sulfonamido)benzoate;
ethyl 3-(5-(5-(trifluoromethyl)isoxazol-3-yl)furan-2-sulfonamido)benzoate;
ethyl 3-(5-(4,5-dimethyl-1H-pyrazol-3-yl)thiophene-2-sulfonamido)benzoate;
ethyl 3-(5-(5-methyl-1H-pyrazol-3-yl)thiophene-2-sulfonamido)benzoate;
ethyl 3-(5-(5-(trifluoromethyl)isoxazol-3-yl)thiophene-2-sulfonamido)benzoate;
ethyl 3-(5-(3-(trifluoromethyl)isoxazol-5-yl)thiophene-2-sulfonamido)benzoate;
ethyl 3-(5-(3-methylisoxazol-5-yl)thiophene-2-sulfonamido)benzoate;
ethyl 3-(4-(4-(tert-butyl)thiazol-2-yl)thiophene-2-sulfonamido)benzoate;
methyl 3-(4-(4-(tert-butyl)thiazol-2-yl)thiophene-2-sulfonamido)benzoate;
methyl 3-(3-(1H-tetrazol-1-yl)phenylsulfonamido)benzoate;
ethyl 3-(2-ethyl-5-(5-(trifluoromethyl)isoxazol-3-yl)phenylsulfonamido)benzoate;
ethyl 3-(2-methyl-5-(5-methyl-1,2,4-oxadiazol-3-yl)phenylsulfonamido)benzoate;
ethyl 3-(3-(5-methyl-1,2,4-oxadiazol-3-yl)phenylsulfonamido)benzoate;
ethyl 3-(2-methyl-5-(5-methyl-1H-pyrazol-3-yl)phenylsulfonamido)benzoate;
ethyl 3-(2-methyl-5-(2-methylthiazol-4-yl)phenylsulfonamido)benzoate;
ethyl 3-(2-isopropyl-5-(3-methylisoxazol-5-yl)phenylsulfonamido)benzoate;
ethyl 3-(2-methyl-5-(2-methyloxazol-5-yl)phenylsulfonamido)benzoate;
ethyl 3-(2-ethyl-5-(3-methylisoxazol-5-yl)phenylsulfonamido)benzoate;
ethyl 3-(4-(2-methyloxazol-4-yl)phenylsulfonamido)benzoate;
ethyl 3-(4-(2-methyloxazol-5-yl)phenylsulfonamido)benzoate;
methyl 3-(4-(2,5-dimethyloxazol-4-yl)phenylsulfonamido)benzoate;
ethyl 3-(2-methyl-5-(6-oxo-1,6-dihydropyridazin-3-yl)phenylsulfonamido)benzoate;
3-(6-butoxynaphthalene-2-sulfonamido)benzoic acid;
3-(6-methoxynaphthalene-2-sulfonamido)benzoic acid;
3-(6-propoxynaphthalene-2-sulfonamido)benzoic acid;
3-(6-methylnaphthalene-2-sulfonamido)benzoic acid;
3-(4-(3,5-dimethyl-1H-pyrazol-1-yl)phenylsulfonamido)benzoic acid;
N-(3-(2H-tetrazol-5-yl)phenyl)benzo[c][1,2,5]thiadiazole-4-sulfonamide;
N-(3-(2H-tetrazol-5-yl)phenyl)-2,3,5,6-tetramethylbenzenesulfonamide;
N-(3-(2H-tetrazol-5-yl)phenyl)-2,4,5-trichlorobenzenesulfonamide;
N-(3-(2H-tetrazol-5-yl)phenyl)-5-(tert-butyl)-2-methylbenzenesulfonamide;
3-methyl-N-(3-(oxazol-5-yl)phenyl)quinoline-8-sulfonamide;
5-bromo-2-methyl-N-(3-(oxazol-5-yl)phenyl)benzenesulfonamide;
2,5-dichloro-3,6-dimethyl-N-(3-(oxazol-5-yl)phenyl)benzenesulfonamide;
N-(3-(oxazol-5-yl)phenyl)-2,3-dihydrobenzofuran-5-sulfonamide;
2-chloro-4-methyl-N-(3-(oxazol-5-yl)phenyl)benzenesulfonamide;
2-chloro-4-fluoro-N-(3-(oxazol-5-yl)phenyl)benzenesulfonamide;
2-fluoro-N-(3-(oxazol-5-yl)phenyl)benzenesulfonamide;
N-(3-(oxazol-5-yl)phenyl)quinoline-8-sulfonamide;
N-(3-(oxazol-5-yl)phenyl)naphthalene-2-sulfonamide;
2-bromo-N-(3-(oxazol-5-yl)phenyl)benzenesulfonamide;
5-(dimethylamino)-N-(3-(oxazol-5-yl)phenyl)naphthalene-I-sulfonamide;
2,3,5,6-tetramethyl-N-(3-(oxazol-5-yl)phenyl)benzenesulfonamide;
2,5-dichloro-N-(3-(oxazol-5-yl)phenyl)benzenesulfonamide; or
2,3,4-trifluoro-N-(3-(oxazol-5-yl)phenyl)benzenesulfonamide.

2. The compound of item 1, wherein A is O or S; or a pharmaceutically acceptable salt thereof.

3. The compound of item 2, wherein

R¹ is selected from H; halogen; and C1-C6 alkyl optionally substituted with at least one halogen; and

R² and R³ form, together with the carbon atoms to which they are attached, a benzene ring, optionally substituted with at least one R⁶; or a 5- or 6-membered heteroaromatic or heterocyclic ring, optionally substituted with at least one R⁶; or

R¹ and R² form, together with the carbon atoms to which they are attached, a benzene ring optionally substituted with at least one R⁶; or a 5- or 6-membered heteroaromatic or heterocyclic ring, optionally substituted with at least one R⁶; and

R³ is selected from H; halogen; C1-C6 alkyl optionally substituted with at least one halogen; or a pharmaceutically acceptable salt thereof.

4. The compound of item 2, wherein

R¹ is selected from H; halogen; and C1-C6 alkyl optionally substituted with at least one halogen;

R² and R³ are independently selected from H; halogen; C1-C6 alkyl optionally substituted with at least one halogen; phenyl optionally substituted with at least one R⁶; 5- or 6-membered heteroaryl optionally substituted with at least one R⁶; and 5- or 6-membered aryl-sulfonyl or heteroaryl sulfonyl, optionally substituted with at least one R⁶; provided that—at least one of R² and R³ is selected from said phenyl, heteroaryl, arylsulfonyl and heteroarylsulfonyl, and
when L is (a), neither R² nor R³ is unsubstituted phenyl;

or a pharmaceutically acceptable salt thereof.

5. The compound of item 1, wherein A is a double bond; or a pharmaceutically acceptable salt thereof.

6. The compound of item 5, wherein

R¹ is selected from H; halogen; and C1-C6 alkyl optionally substituted with at least one halogen; and

R² and R³ are independently selected from H, halogen, C1-C6 alkyl optionally substituted with at least one halogen; phenyl optionally substituted with at least one R⁶; 5- or 6-membered heteroaryl optionally substituted with at least one R⁶; and 5- or 6-membered arylsulfonyl or heteroaryl sulfonyl optionally substituted with at least one R⁶;

or a pharmaceutically acceptable salt thereof.

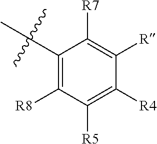

wherein R⁴, R⁵, R⁷, R⁸ and R″ are as defined in item 1; or a pharmaceutically acceptable salt thereof.

8. The compound of item 7, wherein

R⁴ is selected from H and C1-C6 alkyl;

R⁵ is selected from oxazol-5-yl and tetrazol-5-yl, said oxazol-5-yl and tetrazol-5-yl optionally being substituted by R⁹;

R″ is selected from H and C1-C6 alkyl;

R⁷ is selected from H and C1-C6 alkyl; and

R⁸ is selected from H and C1-C6 alkyl;

or a pharmaceutically acceptable salt thereof.

9. The compound of item 7, wherein

R⁴ is selected from H, hydroxy and C1-C6 alkyl;

R⁵ is selected from H and C1-C6 alkyl; and R″ is selected from C0-C1 alkyl-COOR¹²; or R⁵ is COOR¹²; and R″ is selected from H, C1-C6 alkyl, and nitro;

R⁷ is selected from H, C1-C6 alkyl, and nitro; and

R⁸ is selected from H, hydroxy, and C1-C6 alkyl;

or a pharmaceutically acceptable salt thereof.

10. The compound of any one of the items 1 to 6, wherein L is

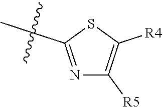

wherein R⁴ and R⁵ are as defined in item 1; or a pharmaceutically acceptable salt thereof.

11. A compound according to item 1, selected from:
2,3,4-trichloro-N-[3-(IH-tetrazol-5-yl)phenyl]benzenesulfonamide;
5-(1,3-oxazol-5-yl)-N-[3-(IH-tetrazol-5-yl)phenyl]thiophene-2-sulfonamide;
5-chloro-3-methyl-N-[3-(1,3-oxazol-5-yl)phenyl]-1-benzothiophene-2-sulfonamide;
5-isopropyl-3-methyl-N-[3-(IH-tetrazol-5-yl)phenyl]benzothiophene-2-sulfonamide;
3-{[(5-isopropyl-3-methyl-I-benzothien-2-yl)sulfonyl]amino}benzoic acid;
2-{[(5-isopropyl-3-methyl-I-benzothien-2-yl)sulfonyl]amino}isonicotinic acid;
2-{[(5-isopropyl-3-methyl-I-benzothien-2-yl)sulfonyl]amino}-1,3-thiazole-5-carboxylic acid;
2-{[(5-isopropyl-3-methyl-I-benzothien-2-yl)sulfonyl]amino}-4-methyl-1,3-thiazole-5-carboxylic acid;
5-{[(5-isopropyl-3-methyl-I-benzothien-2-yl)sulfonyl]amino}nicotinic acid;
6-{[(5-isopropyl-3-methyl-I-benzothien-2-yl)sulfonyl]amino}pyridine-2-carboxylic acid;
3-{[(5-isopropyl-3-methyl-I-benzothien-2-yl)sulfonyl]amino}-5-nitrobenzoic acid;
[5-(3-{[(5-isopropyl-3-methyl-I-benzothien-2-yl)sulfonyl]amino}phenyl)-2H-tetrazol-2-yl]acetic acid;
5-isopropyl-3-methyl-N-[3-(IH-tetrazol-5-yl)phenyl]-1-benzofuran-2-sulfonamide;
3-{[(5-isopropyl-3-methyl-I-benzofuran-2-yl)sulfonyl]amino}benzoic acid;
(3-{[(5-isopropyl-3-methyl-I-benzothien-2-yl)sulfonyl]amino}phenyl)acetic acid;
N-[3-(IH-tetrazol-5-yl)phenyl]biphenyl-3-sulfonamide;
4′-fluoro-N-[3-(IH-tetrazol-5-yl)phenyl]biphenyl-3-sulfonamide;
2,6-dichloro-N-[3-(IH-tetrazol-5-yl)phenyl]-4-(trifluoromethyl)benzenesulfonamide;
4′-methoxy-N-[3-(IH-tetrazol-5-yl)phenyl]biphenyl-3-sulfonamide;
N-[3-(IH-tetrazol-5-yl)phenyl]naphthalene-2-sulfonamide;
5-[2-(methylthio)pyrimidin-4-yl]-N-[3-(IH-tetrazol-5-yl)phenyl]thiophene-2-sulfonamid
5-(5-fluoro-2-methoxyphenyl)-N-[3-(IH-tetrazol-5-yl)phenyl]thiophene-2-sulfonamide; 5-(3,5-difluorophenyl)-N-[3-(IH-tetrazol-5-yl)phenyl]thiophene-2-sulfonamide;
5-(2-methoxyphenyl)-N-[3-(IH-tetrazol-5-yl)phenyl]thiophene-2-sulfonamide;
5-(2-methyl-1,3 hiazol-4-yl)-N-[3-(IH-tetrazol-5-yl)phenyl]thiophene-2-sulfonamide;
5-(2-chlorophenyl)-N-[3-(IH-tetrazol-5-yl)phenyl]thiophene-2-sulfonamide;
5-(4-methylphenyl)-N-[3-(IH-tetrazol-5-yl)phenyl]thiophene-2-sulfonamide;
5-(2,4-dimethoxyphenyl)-N-[3-(IH-tetrazol-5-yl)phenyl]thiophene-2-sulfonamide;
5-(4-chlorophenyl)-N-[3-(IH-tetrazol-5-yl)phenyl]thiophene-2-sulfonamide;
5-(4-fluoro-2-methoxyphenyl)-N-[3-(IH-tetrazol-5-yl)phenyl]thiophene-2-sulfonamide;
N-[3-(IH-tetrazol-5-yl)phenyl]biphenyl-4-sulfonamide;
5-chloro-3-methyl-N-[3-(IH-tetrazol-5-yl)phenyl]-I-benzothiophene-2-sulfonamide;
3,5-dimethyl-N-[3-(IH-tetrazol-5-yl)phenyl]-I-benzothiophene-2-sulfonamide;
5-bromo-3-methyl-N-[3-(IH-tetrazol-5-yl)phenyl]-I-benzothiophene-2-sulfonamide;
7-methoxy-3-methyl-N-[3-(IH-tetrazol-5-yl)phenyl]-I-benzothiophene-2-sulfonamide;
7-chloro-3-methyl-N-[3-(IH-tetrazol-5-yl)phenyl]-I-benzothiophene-2-sulfonamide;
5-methoxy-3-methyl-N-[3-(IH-tetrazol-5-yl)phenyl]-I-benzothiophene-2-sulfonamide;
3-{[(5-chloro-3-methyl-I-benzothien-2-yl)sulfonyl]amino}benzoic acid;
3-methyl-N-[3-(IH-tetrazol-5-yl)phenyl]-I-benzothiophene-2-sulfonamide;
5-methyl-N-[3-(IH-tetrazol-5-yl)phenyl]-I-benzothiophene-2-sulfonamide;
3-{[(5-bromo-3-methyl-I-benzothien-2-yl)sulfonyl]amino}benzoic acid;
3-{[(7-methoxy-3-methyl-I-benzothien-2-yl)sulfonyl]amino}benzoic acid;
5-fluoro-3-methyl-N-[3-(IH-tetrazol-5-yl)phenyl]-I-benzothiophene-2-sulfonamide;
3-{[(7-chloro-3-methyl-I-benzothien-2-yl)sulfonyl]amino}benzoic acid;
3-methyl-5-pyrrolidin-I-yl-N-[3-(IH-tetrazol-5-yl)phenyl]-I-benzothiophene-2-sulfonamide;
3-{[(3-methyl-5-pyrrolidin-I-yl-I-benzothien-2-yl)sulfonyl]amino}benzoic acid;
3-{[(5-amino-3-methyl-I-benzothien-2-yl)sulfonyl]amino}benzoic acid;
5-amino-3-methyl-N-[3-(IH-tetrazol-5-yl)phenyl]-I-benzothiophene-2-sulfonamide;
3-({[5-(acetylamino)-3-methyl-I-benzothien-2-yl]sulfonyl}amino)benzoic acid;
3-(p-tolyl)-N-[3-(IH-tetrazol-5-yl)phenyl]benzenesulfonamide;
2,4-dichloro-5-methyl-N-[3-(IH-tetrazol-5-yl)phenyl]benzenesulfonamide;
3-(3,5-Dichlorophenyl)-N-[3-(I-H-tetrazol-5-yl)phenyl]benzenesulfonamide;
2,4-dichloro-6-methyl-N-[3-(IH-tetrazol-5-yl)phenyl]benzenesulfonamide;
3-[[3-(3, 5-dichlorophenyl)phenyl] sulfonylamino]benzoic acid;
N-[3-(IH-tetrazol-5-yl)phenyl]-3-[4-(trifluoromethyl)phenyl]benzenesulfonamide;
4-bromo-N-[3-(IH-tetrazol-5-yl)phenyl]-2-(trifluoromethyl)benzenesulfonamide;
4-bromo-2-fluoro-N-[3-(IH-tetrazol-5-yl)phenyl]benzenesulfonamide;
3-[5-[[3-(IH-tetrazol-5-yl)phenyl]sulfamoyl]-2-thienyl]benzamide;
5-(5-chloro-I,2,44hiadiazol-3-yl)-N-[3-(IH-tetrazol-5-yl)phenyl]thiophene-2-sulfonamide; 5-phenyl-N-[3-(IH-tetrazol-5-yl)phenyl]thiophene-2-sulfonamide;
3-[[5-(2-methylsulfanylpyrimidin-4-yl)-2-thienyl]sulfonylamino]benzoic acid;
3-[[5-(2-methyl-I,3-thiazol-4-yl)-2-thienyl]sulfonylamino]benzoic acid;
N-[3-(IH4etrazol-5-yl)phenyl]-5-[5-(trifluoromethyl)isoxazol-3-yl]thiophene-2-sulfonamide; N-[3-(IH-tetrazol-5-yl)phenyl]benzofuran-2-sulfonamide;
5-isoxazol-3-yl-N-[3-(IH-tetrazol-5-yl)phenyl]thiophene-2-sulfonamide;
2,4,6-trichloro-N-[3-(IH-tetrazol-5-yl)phenyl]benzenesulfonamide;
2.3-dichloro-N-[3-(IH-tetrazol-5-yl)phenyl]benzenesulfonamide;
2,5-dichloro-N-[3-(IH-tetrazol-5-yl)phenyl]benzenesulfonamide;
3-chloro-2-methyl-N-[3-(IH-tetrazol-5-yl)phenyl]benzenesulfonamide;
2.4-dichloro-N-[3-(IH-tetrazol-5-yl)phenyl]benzenesulfonamide;
2,4,5-trichloro-N-[3-(IH-tetrazol-5-yl)phenyl]benzenesulfonamide;
2,4-difluoro-N-[3-(IH-tetrazol-5-yl)phenyl]benzenesulfonamide;
7-chloro-N-[3-(IH-tetrazol-5-yl)phenyl]-2, I,3-benzoxadiazole-4-sulfonamide;
methyl 3-{[(5-chloro-3-methyl-I-benzothien-2-yl)sulfonyl]amino}benzoate
3-{[(3-methyl-I-benzothiophen-2-yl)sulfonyl]amino}benzoic acid;
3-{[(5-fluoro-3-methyl-I-benzothiophen-2-yl)sulfonyl]amino}benzoic acid;
3-{[(5-methoxy-3-methyl-I-benzothiophen-2-yl)sulfonyl]amino}benzoic acid;
3-{[(5-pyridin-2-yl-2-thienyl)sulfonyl]amino}benzoic acid;
3-{[(5-isoxazol-3-yl-2-thienyl)sulfonyl]amino}benzoic acid;
3-[(biphenyl-3-ylsulfonyl)amino]benzoic acid;
2-chloro-4-fluoro-N-[3-(IH-tetrazol-5-yl)phenyl]benzenesulfonamide;
N-[3-(IH-tetrazol-5-yl)phenyl]-I-benzothiophene-2-sulfonamide;
4-(I,3-oxazol-5-yl)-N-[3-(IH-tetrazol-5-yl)phenyl]benzenesulfonamide;
3-[(I-benzothien-2-ylsulfonyl)amino]benzoic acid;
4′-methoxy-N-[3-(IH-tetrazol-5-yl)phenyl]biphenyl-4-sulfonamide;
3′,4′-dichloro-N-[3-(IH-tetrazol-5-yl)phenyl]biphenyl-4-sulfonamide;
5-isoxazol-5-yl-N-[3-(IH-tetrazol-5-yl)phenyl]thiophene-2-sulfonamide;
methyl 3-({[5-(2-methyl-I,3-thiazol-4-yl)-2-thienyl]sulfonyl}amino)benzoate
3-{[(5-methyl-I-benzothiophen-2-yl)sulfonyl]amino}benzoic acid;
4′-chloro-N-[3-(IH-tetrazol-5-yl)phenyl]biphenyl-3-sulfonamide;
3′,4′-dichloro-N-[3-(IH-tetrazol-5-yl)phenyl]biphenyl-3-sulfonamide;
methyl 3-({[3-methyl-5-(I-methylethyl)-benzothiophen-2-yl]sulfonyl}amino)benzoate
3-{[(4′-chlorobiphenyl-3-yl)sulfonyl]amino}benzoic acid;
3-{[(4′-fluorobiphenyl-3-yl)sulfonyl]amino}benzoic acid;
3-{[(4′-methoxybiphenyl-3-yl)sulfonyl]amino}benzoic acid;
3-{[(3′,4′-dichlorobiphenyl-3-yl)sulfonyl]amino}benzoic acid;
5-(3-methoxyphenyl)-N-[3-(IH-tetrazol-5-yl)phenyl]thiophene-2-sulfonamide;
5-(3,4-dichlorophenyl)-N-[3-(IH-tetrazol-5-yl)phenyl]thiophene-2-sulfonamide;
N-[3-(IH-tetrazol-5-yl)phenyl]-5-[3-(trifluoromethyl)phenyl]thiophene-2-sulfonamide;
5-(2-methylphenyl)-N-[3-(IH-tetrazol-5-yl)phenyl]thiophene-2-sulfonamide
5-(2,4-difluorophenyl)-N-[3-(IH-tetrazol-5-yl)phenyl]thiophene-2-sulfonamide;
5-(3-chloro-4-fluorophenyl)-N-[3-(IH-tetrazol-5-yl)phenyl]thiophene-2-sulfonamide;
5-(3-chlorophenyl)-N-[3-(IH-tetrazol-5-yl)phenyl]thiophene-2-sulfonamide;
5-pyridin-4-yl-N-[3-(IH-tetrazol-5-yl)phenyl]thiophene-2-sulfonamide;
3-{[(3-methyl-5-morpholin-4-yl-I-benzothiophen-2-yl)sulfonyl]amino}benzoic acid;
2-(IH-pyrrol-I-yl)ethyl 3-{[(5-chloro-3-methyl-I-benzothien-2-yl)sulfonyl]amino}benzoate ethyl 3-{[(5-chloro-3-methyl-I-benzothien-2-yl)sulfonyl]amino}benzoate
isopropyl 3-{[(5-chloro-3-methyl-1-benzothien-2-yl)sulfonyl]amino}benzoate
2-methoxy ethyl 3-{[(5-chloro-3-methyl-I-benzothiophen-2-yl)sulfonyl]amino}benzoate butyl 3-{[(5-chloro-3-methyl-I-benzothiophen-2-yl)sulfonyl]amino}benzoate
benzyl 3-{[(5-chloro-3-methyl-I-benzothiophen-2-yl)sulfonyl]amino}benzoate
propyl 3-{[(5-chloro-3-methyl-1-benzothien-2-yl)sulfonyl]amino}benzoate
pentyl 3-{[(5-chloro-3-methyl-1-benzothiophen-2-yl)sulfonyl]amino}benzoate
hexyl 3-{[(5-chloro-3-methyl-1-benzothiophen-2-yl)sulfonyl]amino}benzoate
phenyl 3-{[(5-chloro-3-methyl-1-benzothiophen-2-yl)sulfonyl]amino}benzoate
tetrahydrofuran-3-yl 3-{[(5-chloro-3-methyl-1-benzothiophen-2-yl)sulfonyl]amino}benzoate; tetrahydrofuran-3-ylmethyl 3-{[(5-chloro-3-methyl-1-benzothiophen-2-yl)sulfonyl]amino}benzoate;
3-(dimethylamino)propyl 3-{[(5-chloro-3-methyl-1-benzothiophen-2-yl)sulfonyl]amino}benzoate;
methyl {5-[3-({[3-methyl-5-(I-methylethyl)-I-benzothiophen-2-yl]sulfonyl}amino)phenyl]-2H-tetrazol-2-yl}acetate;
methyl 3-{[(5-bromo-3-methyl-1-benzothiophen-2-yl)sulfonyl]amino}benzoate;
methyl 3-{[(7-methoxy-3-methyl-1-benzothiophen-2-yl)sulfonyl]amino}benzoate;
methyl 3-{[(7-chloro-3-methyl-1-benzothiophen-2-yl)sulfonyl]amino}benzoate;
methyl 3-({[3-methyl-5-(I-methylethyl)-I-benzofuran-2-yl]sulfonyl}amino)benzoate;
methyl 2-({[3-methyl-5-(1-methylethyl)-1-benzothiophen-2-yl]sulfonyl} amino)-1,3-thiazole-5-carboxylate;
ethyl 4-methyl-2-({[3-methyl-5-(1-methylethyl)-1-benzothiophen-2-yl] sulfonyl} amino)-1,3-thiazole-5-carboxylate;
ethyl 2-{[(5-chloro-3-methyl-1-benzothiophen-2-yl)sulfonyl]amino}-4-methyl-1,3-thiazole-5-carboxylate;
ethyl 2-({[5-chloro-4-(2,5-difluorophenyl)thiophen-2-yl]sulfonyl}amino)-4-methyl-1,3-thiazole-5-carboxylate;
2-{[(5-chloro-3-methyl-1-benzothiophen-2-yl)sulfonyl]amino}-4-methyl-1,3-thiazole-5-carboxylic acid;
2-({[5-chloro-4-(2,5-difluorophenyl)thiophen-2-yl]sulfonyl}amino)-4-methyl-1,3-thiazole-5-carboxylic acid;
2-{[(5-chloro-3-methyl-1-benzothiophen-2-yl)sulfonyl]amino}-1,3-thiazole-5-carboxylic ac-id;
2-({[5-(3,5-difluorophenyl)thiophen-2-yl]sulfonyl}amino)-5-methyl-1,3-thiazole-4-carboxylic acid;
2-({[5-chloro-4-(2,5-difluorophenyl)thiophen-2-yl]sulfonyl}amino)-5-methyl-1,3-thiazole-4-carboxylic acid;
5-methyl-2-({[3-methyl-5-(1-methylethyl)-I-benzothiophen-2-yl]sulfonyl}amino)-1,3-thiazole-4-carboxylic acid;
3-{[(5-chloro-3-methyl-1-benzothiophen-2-yl)sulfonyl]amino}-2-hydroxybenzoic acid;
3-({[5-chloro-4-(2,5-difluorophenyl)thiophen-2-yl]sulfonyl}amino)-2-hydroxybenzoic acid; 3-({[5-chloro-4-(2,3-dihydro-1-benzofuran-5-yl)thiophen-2-yl]sulfonyl}amino)-2-hydroxybenzoic acid;
3-({[5-chloro-4-(2-hydroxyphenyl)thiophen-2-yl]sulfonyl}amino)-2-hydroxybenzoic acid;
5-{[(5-chloro-3-methyl-1-benzothiophen-2-yl)sulfonyl]amino}-2-hydroxybenzoic acid;
5-{[(4′-chlorobiphenyl-3-yl)sulfonyl]amino}-2-hydroxybenzoic acid;
methyl 5-{[(5-chloro-3-methyl-1-benzothiophen-2-yl)sulfonyl]amino}-2-hydroxybenzoate;
5-chloro-3-methyl-N-[3-(1,3-oxazol-5-yl)phenyl]-1-benzothiophene-2-sulfonamide;
5-(phenylsulfonyl)-N-[3-(IH-tetrazol-5-yl)phenyl]thiophene-2-sulfonamide;
2,2-dimethyl-N-[3-(IH-tetrazol-5-yl)phenyl]chromane-6-sulfonamide;
or a pharmaceutically acceptable salt thereof.

13. A pharmaceutical composition comprising a compound according to any one of the items 1-11 or a pharmaceutically acceptable salt thereof and at least on pharmaceutically acceptable excipient.

15. A compound of formula (I)

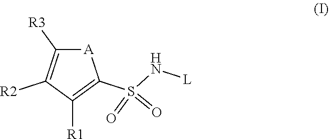

wherein: (i) A is O or S; and R¹ is selected from H; halogen; and C1-C6 alkyl optionally substituted with at least one halogen; or

R¹ and R² form, together with the carbon atoms to which they are attached, a benzene ring optionally substituted with at least one R⁶; or a 5- or 6-membered heteroaromatic or heterocyclic ring, optionally substituted with at least one R⁶;

R² and R³ are independently selected from H; halogen; C1-C6 alkyl optionally substituted with at least one halogen; phenyl optionally substituted with at least one R⁶; 5- or o-membered heteroaryl optionally substituted with at least one R⁶; and 5- or 6-membered aryl-sulfonyl or heteroarylsulfonyl, optionally substituted with at least one R⁶; provided that—at least one of R² and R³ is selected from said phenyl, heteroaryl, aryl sulfonyl and heteroarylsulfonyl, and
when L is (a), neither R nor R is unsubstituted phenyl; or

R and R form, together with the carbon atoms to which they are attached, a benzene ring optionally substituted with at least one R⁶; or a 5- or 6-membered heteroaromatic or heterocyclic ring, optionally substituted with at least one R⁶; or

(ii) A is CR′═CR′;

each R′ is independently selected from H; halogen; and C1-C6 alkyl optionally substituted with at least one halogen;

R¹ is selected from H; halogen; and C1-C6 alkyl optionally substituted with at least one halogen; or

R¹ and R² form, together with the carbon atoms to which they are attached, a benzene ring optionally substituted with at least one R⁶; or a 5- or 6-membered heteroaromatic or heterocyclic ring, optionally substituted with at least one R⁶;

R² and R³ are independently selected from H, halogen, C1-C6 alkyl optionally substituted with at least one halogen; phenyl optionally substituted with at least one R⁶; 5- or 6-membered heteroaryl optionally substituted with at least one R⁶; and 5- or 6-membered aryl-sulfonyl or heteroarylsulfonyl, optionally substituted with at least one R⁶; provided that:
when both R and R are selected from H, halogen and C1-C6 alkyl optionally substituted with at least one halogen, the ring containing A is substituted in ortho position relative to the sulphonamide bond with at least one substituent selected from halogen and C1-C6 alkyl optionally substituted with at least one halogen;
when L is (a), neither R² nor R³ is unsubstituted phenyl; and
when L is (c), R is optionally substituted phenyl only when R is tetrazol-5-yl, and R is unsubstituted phenyl only when R⁴ is not hydroxy; or

R and R form, together with the carbon atoms to which they are attached, a benzene ring optionally substituted with at least one R⁶, provided that said benzene ring is unsubstituted only when R⁵ is tetrazolyl or oxazolyl; or a 5- or 6-membered heteroaromatic or heterocyclic ring, optionally substituted with at least one R⁶;

L is

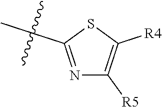

wherein R⁴ is COOR¹²; and R⁵ is selected from H and C1-C6 alkyl; or

R⁴ is selected from H and C1-C6 alkyl; and R⁵ is COOR¹²; or

(b)

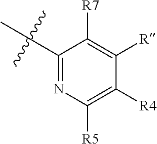

wherein R⁴ is selected from H and C1-C6 alkyl; R⁵ is selected from H and C1-C6 alkyl; and R″ is selected from C0-C1 alkyl-COOR¹²; or

R⁵ is selected from COOR¹²; and R″ is selected from H and C1-C6 alkyl; and

selected from H, C1-C6 alkyl, and nitro;

(c)

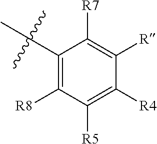

wherein R⁴ is selected from H, hydroxy and C1-C6 alkyl; R⁵ is selected from H, C1-C6 alkyl; and R″ is selected from C0-C1 alkyl-COOR¹²; or

512

R is selected from COOR, oxazol-5-yl and tetrazol-5-yl, said oxazol-5-yl and tetrazol-5-yl optionally being substituted by R⁹; and

R″ is selected from H, C1-C6 alkyl, and nitro;

selected from H, C1-C6 alkyl, and nitro; and

R is selected from H, hydroxy, and C1-C6 alkyl; or

(d)

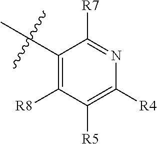

wherein selected from H and C1-C6 alkyl; and R⁵ is COOR¹²

R is selected from H, C1-C6 alkyl, and nitro; and

R is selected from H, hydroxy, and CI-C6 alkyl;

provided that in any of (a), (b), (c) and (d), R⁴, R⁵ and R″ are selected from C0-C1 alkyl —COOR¹² only when at least one of R² or R³ is optionally substituted phenyl or optionally substituted heteroaryl; or when R and R together with the carbon atoms to which they are attached form a benzene ring optionally substituted by at least one R⁶;

R⁶ is selected from C1-C6 alkyl, cyano, halogen, hydroxy, C1-C6 alkoxy, C1-C6 alkylthio, tetrahydropyrrolyl, R¹⁰RⁿN, carbamoyl, and C1-C6 alkylcarbonylamino, or is an ethyleneoxy biradical forming, together with the atoms to which it is attached, a five-membered oxygen containing cycle; wherein any alkyl is optionally substituted with at least one halogen;

R⁹ is selected from C0-C1 alkyl-COOR¹²;

R¹⁰ and R¹¹ are independently selected from H and C1-C6 alkyl or form, together with the nitrogen to which they are attached, a 5- or 6-membered cyclic amino optionally containing one other cyclic heteroatom;

R¹² is selected from H, C1-C6 alkyl; heteroaryl-C0-C2 alkyl; (C1-C3 alkoxy)ₚC1-C3 alkyl; aryl-C0-C2 alkyl; heterocyclyl-C0-C2 alkyl; and C1-C6 dialkylamino-C1-C6 alkyl, wherein any cyclic moiety is optionally substituted with C1-C6 alkyl;

p is 1 or 2;

or a pharmaceutically acceptable salt thereof;

for use in therapy;

provided that the compound is not:

methyl 6-(4′-cyano-[1,-biphenyl]-4-ylsulfonamido)picolinate;

ethyl 3-(5-(4,5-dimethyl-1H-pyrazol-3-yl)thiophene-2-sulfonamido)benzoate; or

ethyl 3-(5-(5-methyl-1H-pyrazol-3-yl)thiophene-2-sulfonamido)benzoate.

16. A pharmaceutical composition comprising the compound according to item 15 or a pharmaceutically acceptable salt thereof and at least on pharmaceutically acceptable excipient.

Item C

PFKFB3 inhibitor for use in neuroprotection, wherein PFKFB3 is selected from those described in WO2011161201 A1 incorporated here by reference.

PFKFB3 inhibitor for use in neuroprotection, wherein PFKFB3 inhibitor is selected from the following

1. A compound of formula (I)

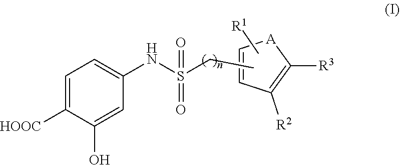

wherein: n is 0 or 1; A is O, S, —CR⁴═CR⁴— or —CR⁴═N—;

R¹ is selected from H; halogen; C₁-C₆ alkyl, optionally substituted with at least one halogen; and C₁-C₆ alkoxy, substituted with at least one halogen;

R² and R³ are each independently selected from H; halogen; C₁-C₆ alkyl; C₁-C₆ alkoxy; secondary or tertiary C₁-C₆ alkylamido; carbocyclylcarbonylamino-C₁-C₂ alkyl; 5- or 6-membered cyclic aminocarbonyl; C₁-C₆ alkylcarbonylamino; C₁-C₆ alkylsulfonyl; hydroxy-C₀-C₆ alkyl, C₁-C₆ alkylcarbonyl; carboxy; C₁-C₆ alkoxycarbonyl; cyano; nitro; carbocyclyloxy; heterocyclyloxy; carbocyclyl-C₀-C₃ alkyl; carbocyclyl-C₂-C₃ alkenyl; heterocyclyl-C₀-C₃ alkyl; and heterocyclyl-C₂-C₃ alkenyl;

wherein any alkyl is optionally substituted with at least one halogen; any carbocyclyl or heterocyclyl is 5- or 6-membered monocyclyl or 9- or 10-membered bicyclyl; and any carbocyclyl or heterocyclyl is optionally substituted with at least one R⁵; or R² and R³ form, together with the carbon atoms to which they are attached, a 5- or 6-membered carbocyclic or heterocyclic ring, which ring is optionally substituted with at least one R⁵;

each R⁴ is independently selected from H, halogen, monocyclic C₃-C₆ carbocyclyl and C₁-C₆ alkyl, wherein any alkyl is optionally substituted with at least one halogen;

each R⁵ is independently selected from halogen; C₁-C₆ alkyl; C₁-C₆ alkoxy; phenoxy; amino; cyano; nitro; secondary or tertiary C₁-C₆ alkylamino; 5- or 6-membered cyclic amino; C₁-C₆ alkylcarbonylamino; carbamoyl; secondary or tertiary C₁-C₆ alkylamido; 5- or 6-membered cyclic aminocarbonyl; C₁-C₆ alkoxycarbonylamino; hydroxy-C₀-C₆ alkyl; C₁-C₆-alkylthio; carboxy-C₀-C₆-alkyl; C₁-C₆ alkoxycarbonyl; C₁-C₆ alkylcarbonyl; C₁-C₆-alkylsulfonyl; and C₁-C₆ alkylsulfonylamino; wherein any alkyl is optionally substituted with at least one halogen; or a pharmaceutically acceptable salt thereof, for use as a medicament, with the proviso that when A is CR⁴═CR⁴ and n is 0, then neither R² nor R³ is selected from 4-hydroxypyrazolo[1,5-a]-1,3,5-triazin-8-yl and 2,4-dihydroxypyrazolo[1,5-a]-1,3,5-triazin-8-yl; the compound is not selected from
4-(3,4-dichlorophenylsulfonamido)-2-hydroxybenzoic acid,
4-(2,5-dichlorophenylsulfonamido)-2-hydroxybenzoic acid,
4-(2,5-diethylphenylsulfonamido)-2-hydroxybenzoic acid,
4-(4-bromophenylsulfonamido)-2-hydroxybenzoic acid,
4-(3-carboxy-4-hydroxyphenylsulfonamido)-2-hydroxybenzoic acid,
2-hydroxy-4-(4-methylphenylsulfonamido)benzoic acid,
2-hydroxy-4-(phenylsulfonamido)benzoic acid, and
4-(4-ethylphenylsulfonamido)-2-hydroxybenzoic acid.

2. The compound according to item 1, wherein one of R² and R³ is selected from carbocyclyl-C0-C3 alkyl; carbocyclyl-C2-C3 alkenyl; heterocyclyl-C0-C3 alkyl; and heterocyclyl-

C2-C3 alkenyl; wherein any carbocyclyl or heterocyclyl is 5- or 6-membered monocyclyl or 9- or 10-membered bicyclyl; and any carbocyclyl or heterocyclyl is optionally substituted with at least one R⁵; or R² and R³ form, together with the carbon atoms to which they are attached, a 5- or 6-membered carbocyclic or heterocyclic ring, which ring is optionally substituted with at least one R⁵; or a pharmaceutically acceptable salt thereof.

3. The compound according to item 2, wherein R² is phenyl, optionally substituted with at least one R⁵, or R² and R³ form, together with the carbon atoms to which they are attached, a benzene ring, optionally substituted with at least one R⁵; or a pharmaceutically acceptable salt thereof.

4. The compound according to any item 1, wherein A is O or S; or a pharmaceutically acceptable salt thereof.

5. The compound according to item 4, wherein

R² is selected from H, C1-C6 alkyl, halogen, and carbocyclyl-C0-C3 alkyl, carbocyclyl-C2-C3 alkenyl, heterocyclyl-C0-C3 alkyl, heterocyclyl-C2-C3 alkenyl; wherein any carbocyclyl or heterocyclyl is optionally substituted with at least one R⁵; and

R³ is selected from H, C1-C6 alkyl, and halogen, wherein any alkyl is optionally substituted with at least one halogen; or a pharmaceutically acceptable salt thereof.

6. The compound according to any one of the items 1-3, wherein A is CR⁴═CR⁴; or a pharmaceutically acceptable salt thereof.

7. A compound according to item 1, selected from
4-[(biphenyl-3-ylsulfonyl)amino]-2-hydroxybenzoic acid,
4-{[(3-bromophenyl)sulfonyl] amino}-2-hydroxybenzoic acid,
4-({[3-(5-acetyl-2-thienyl)phenyl]sulfonyl}amino)-2-hydroxybenzoic acid,
2-hydroxy-4-{[(4′-hydroxybiphenyl-3-yl)sulfonyl]amino}benzoic acid,
4-[({3-[(E)-2-(4-fluorophenyl)vinyl]phenyl} sulfonyl)amino]-2-hydroxybenzoic acid, 4-{[(3′-amino-4′-methoxybiphenyl-3-yl)sulfonyl] amino}-2-hydroxybenzoic acid,
2-hydroxy-4-{[(3-pyridin-3-ylphenyl)sulfonyl] amino} benzoic acid,
4-({[4′-(dimethylamino)biphenyl-3-yl]sulfonyl}amino)-2-hydroxybenzoic acid,
2-hydroxy-4-({[5-(trifluoromethyl)biphenyl-3-yl]sulfonyl}amino)benzoic acid,
4-{[(4,6-difluorobiphenyl-3-yl)sulfonyl]amino}-2-hydroxybenzoic acid,
2-hydroxy-4-{[(6-methoxybiphenyl-3-yl)sulfonyl]amino}benzoic acid,
4-{[(5-chloro-4-phenyl-2-thienyl)sulfonyl]amino}-2-hydroxybenzoic acid,
2-hydroxy-4-({[2′-(hydroxymethyl)biphenyl-3-yl]sulfonyl}amino)benzoic acid,
4-{[(3′-fluorobiphenyl-3-yl)sulfonyl]amino}-2-hydroxybenzoic acid,
4-{[(2′,6′-difluorobiphenyl-3-yl)sulfonyl]amino}-2-hydroxybenzoic acid,
2-hydroxy-4-({[3′-(isopropoxycarbonyl)biphenyl-3-yl]sulfonyl}amino)benzoic acid, 4-({[3-(2,3-dihydro-1-benzofuran-5-yl)phenyl]sulfonyl} amino)-2-hydroxybenzoic acid, 4-{[(3′-fluoro-4′-hydroxybiphenyl-3-yl)sulfonyl]amino}-2-hydroxybenzoic acid,
2-hydroxy-4-{[(3-quinolin-6-ylphenyl)sulfonyl]amino}benzoic acid, 4-{[(3′-aminobiphenyl-3-yl)sulfonyl]amino}-2-hydroxybenzoic acid,
2-hydroxy-4-({[3-(2-methyl-1,3-thiazol-4-yl)phenyl]sulfonyl}amino)benzoic acid, 4-{[(5-chloro-2-thienyl)sulfonyl]amino}-2-hydroxybenzoic acid,
4-({[5-chloro-4-(2,3-dihydro-1-benzofuran-5-yl)-2-thienyl]sulfonyl} amino)-2−hydroxybenzoic acid,
4-({[5-chloro-4-(3-fluoro-4-hydroxyphenyl)-2-thienyl]sulfonyl}amino)-2-hydroxybenzoic acid,
4-{[(5-chloro-4-quinolin-6-yl-2-thienyl)sulfonyl]amino}-2-hydroxybenzoic acid,
4-({[4-(1,3-benzodioxol-5-yl)-5-chloro-2-thienyl]sulfonyl}amino)-2-hydroxybenzoic acid, 4-({[5-chloro-4-(4-hydroxy-3,5-dimethylphenyl)-2-thienyl]sulfonyl}amino)-2-hydroxybenzoic acid,
4-({[5-chloro-4-(2,4-difluorophenyl)-2-thienyl]sulfonyl}amino)-2-hydroxybenzoic acid, 4-({[4-(3-acetylphenyl)-5-chloro-2-thienyl]sulfonyl}amino)-2-hydroxybenzoic acid, 4-[({5-chloro-4-[2-(hydroxymethyl)phenyl]-2-thienyl}sulfonyl)amino]-2-hydroxybenzoic acid,
4-({[5-chloro-4-(3-fluorophenyl)-2-thienyl]sulfonyl}amino)-2-hydroxybenzoic acid, 4-[({5-chloro-4-[3-(isopropoxycarbonyl)phenyl]-2-thienyl}sulfonyl)amino]-2-hydroxybenzoic acid,
4-({[5-chloro-4-(3,5-difluorophenyl)-2-thienyl]sulfonyl}amino)-2-hydroxybenzoic acid, 4-({[5-chloro-4-(6-ethoxypyridin-3-yl)-2-thienyl]sulfonyl}amino)-2-hydroxybenzoic acid, 4-({[4-(3-aminophenyl)-5-chloro-2-thienyl]sulfonyl}amino)-2-hydroxybenzoic acid, 4-({[5-chloro-4-(4-methoxyphenyl)-2-thienyl]sulfonyl}amino)-2-hydroxybenzoic acid, 4-({[4-(4-aminophenyl)-5-chloro-2-thienyl]sulfonyl}amino)-2-hydroxybenzoic acid, tri-fluoroacetate (salt)
4-({[5-chloro-4-(4-hydroxyphenyl)-2-thienyl]sulfonyl}amino)-2-hydroxybenzoic acid,
4-[({5-chloro-4-[3-(hydroxymethyl)phenyl]-2-thienyl}sulfonyl)amino]-2-hydroxybenzoic acid,
4-[({5-chloro-4-[4-(hydroxymethyl)phenyl]-2-thienyl}sulfonyl)amino]-2-hydroxybenzoic acid,
4-({[4-(3-amino-4-methoxyphenyl)-5-chloro-2-thienyl]sulfonyl}amino)-2-hydroxybenzo acid,
trifluoroacetate (salt)
4-{[(5-chloro-4-{4-[(methylsulfonyl)amino]phenyl}-2-thienyl)sulfonyl] amino}-2-hydroxybenzoic acid,
4-{[(7-chloro-3-methyl-1-benzothien-2-yl)sulfonyl]amino}-2-hydroxybenzoic acid, 4-{[(5-chloro-3-methyl-1-benzothien-2-yl)sulfonyl]amino}-2-hydroxybenzoic acid, 2-hydroxy-4-{[(3-piperidin-1-ylphenyl)sulfonyl]amino}benzoic acid,
4-{[(3-acetylphenyl)sulfonyl] amino}-2-hydroxybenzoic acid,
4-{[(3-tert-butylphenyl)sulfonyl]amino}-2-hydroxybenzoic acid,
2-hydroxy-4-{[(4-phenyl-2-thienyl)sulfonyl]amino}benzoic acid,
2-hydroxy-4-[[3-(piperidine-1-carbonyl)phenyl]sulfonylamino]benzoic acid,
2-hydroxy-4-[[3-(methylcarbamoyl)phenyl] sulfonylamino]benzoic acid,
2-hydroxy-4-[[3-(4-methylsulfonylphenyl)phenyl] sulfonylamino]benzoic acid,
4-[[3-(3-ethoxyphenyl)phenyl] sulfonylamino]-2-hydroxy-benzoic acid,
4-[[3-(3-acetamidophenyl)phenyl]sulfonylamino]-2-hydroxy-benzoic acid,
4-[[3-(3,4-dichlorophenyl)phenyl]sulfonylamino]-2-hydroxy-benzoic acid,
4-[[3-(3-carbamoylphenyl)phenyl] sulfonylamino]-2-hydroxy-benzoic acid,
4-[[3-(3-cyanophenyl)phenyl] sulfonylamino]-2-hydroxy-benzoic acid,
2-hydroxy-4-[[3-(4-nitrophenyl)phenyl] sulfonylamino]benzoic acid,
2-hydroxy-4-[[3-[3-(trifluoromethyl)phenyl]phenyl]sulfonylamino]benzoic acid, 2-hydroxy-4-[[3-(4-methylsulfanylphenyl)phenyl]sulfonylamino]benzoic acid,
2-hydroxy-4-[[3-[4-(trifluoromethoxy)phenyl]phenyl]sulfonylamino]benzoic acid, 4-[[3-(2-acetylphenyl)phenyl] sulfonylamino]-2-hydroxy-benzoic acid,
2-hydroxy-4-[[3-(4-phenoxyphenyl)phenyl] sulfonylamino]benzoic acid,
2-hydroxy-4-[[3-(4-hydroxy-3-methoxy-phenyl)phenyl]sulfonylamino]benzoic acid, 2-hydroxy-4-[(3-methylsulfonylphenyl)sulfonylamino]benzoic acid,
4-[[3-(benzofuran-2-yl)phenyl] sulfonylamino]-2-hydroxy-benzoic acid,
2-hydroxy-4-[[3-[4-(methoxycarbonylamino)phenyl]phenyl] sulfony Iamino]benzoic acid, 4-[(5-fluoro-2-methylbenzene)sulfonamido]-2-hydroxybenzoic acid,
4-[(2-bromo-4-iodobenzene)sulfonamido]-2-hydroxybenzoic acid,
2-hydroxy-4-[(2,4,5-trichlorobenzene)sulfonamido]benzoic acid,
2-hydroxy-4-{[4-(I,3-oxazol-5-yl)benzene]sulfonamido}benzoic acid,
4-(2, 1,3-benzothiadiazole-4-sulfonamido)-2-hydroxybenzoic acid,
4-(2, 1,3-benzoxadiazole-4-sulfonamido)-2-hydroxybenzoic acid,
4-{[3-(4-chlorophenyl)benzene]sulfonamido}-2-hydroxybenzoic acid,
2-hydroxy-4-({3-[4-(trifluoromethyl)phenyl]benzene} sulfonamido)benzoic acid, 4-{[3-(4-fluorophenyl)benzene] sulfonamido}-2-hydroxybenzoic acid,
4-{[3-(3,5-dichlorophenyl)benzene]sulfonamido}-2-hydroxybenzoic acid,
2-hydroxy-4-{[3-(4-methoxyphenyl)benzene]sulfonamido}benzoic acid, 2-hydroxy-4-{[3-(4-methylphenyl)benzene]sulfonamido}benzoic acid,
2-hydroxy-4-{[3-(trifluoromethyl)benzene]sulfonamido}benzoic acid,
4-(1-benzothiophene-2-sulfonamido)-2-hydroxybenzoic acid,
2-hydroxy-4-(5-methyl-1-benzothiophene-2-sulfonamido)benzoic acid,
2-hydroxy-4-(7-methoxy-3-methyl-1-benzothiophene-2-sulfonamido)benzoic acid,
2-hydroxy-4-(5-methoxy-3-methyl-1-benzothiophene-2-sulfonamido)benzoic acid,
2-hydroxy-4-[3-methyl-5-(propan-2-yl)-1-benzofuran-2-sulfonamido]benzoic acid,
4-(5-fluoro-3-methyl-1-benzothiophene-2-sulfonamido)-2-hydroxybenzoic acid,
4-{[3-(2H-1,3-benzodioxol-5-yl)benzene]sulfonamido}-2-hydroxybenzoic acid,
4-{[3-(2,4-difluorophenyl)benzene]sulfonamido}-2-hydroxybenzoic acid,
2-hydroxy-4-{[3-(2-nitrophenyl)benzene]sulfonamido}benzoic acid,
2-hydroxy-4-{[3-(4-hydroxy-3,5-dimethylphenyl)benzene]sulfonamido}benzoic acid, 4-{[3-(4-butylphenyl)benzene] sulfonamido}-2-hydroxybenzoic acid,
4-({3-[4-(ethanesulfonyl)phenyl]benzene}sulfonamido)-2-hydroxybenzoic acid,
2-hydroxy-4-{[3-(4-methoxy-3-methylphenyl)benzene] sulfonamido} benzoic acid,
2-hydroxy-4-{[3-(3-hydroxyphenyl)benzene] sulfonamido} benzoic acid,
2-hydroxy-4-{[3-(3-methanesulfonylphenyl)benzene]sulfonamido} benzoic acid,
4-({3-[4-(dimethylcarbamoyl)phenyl]benzene} sulfonamido)-2-hydroxybenzoic acid, 4-{[3-(4-ethylphenyl)benzene] sulfonamido}-2-hydroxybenzoic acid,
4-[(3-{4-[bis(propan-2-yl)carbamoyl]phenyl}benzene)sulfonamido]-2-hydroxybenzoic acid, 4-{[3-(4-acetylphenyl)benzene] sulfonamido}-2-hydroxybenzoic acid,
4-{[3-(2,3-dimethoxyphenyl)benzene] sulfonamido}-2-hydroxybenzoic acid,
4-{[3-(4-fluoro-2-methoxyphenyl)benzene]sulfonamido}-2-hydroxybenzoic acid,
2-hydroxy-4-{[3-(2,3,6-trifluorophenyl)benzene]sulfonamido}benzoic acid,
4-({3-[4-(2-carboxyethyl)phenyl]benzene} sulfonamido)-2-hydroxybenzoic acid,
2-hydroxy-4-{[3-(3-methylphenyl)benzene]sulfonamido} benzoic acid,
4-{[3-(3,5-difluorophenyl)benzene] sulfonamido}-2-hydroxybenzoic acid,
2-hydroxy-4-{[3-(4-methoxy-3,5-dimethylphenyl)benzene] sulfonamido} benzoic acid, 2-hydroxy-4-{[3-(2-methylphenyl)benzene] sulfonamido} benzoic acid,
2-hydroxy-4-{[3-(2-propoxypyridin-3-yl)benzene]sulfonamido}benzoic acid,
4-{[3-(6-ethoxypyridin-3-yl)benzene]sulfonamido}-2-hydroxybenzoic acid,
2-hydroxy-4-({3-[4-(propan-2-yloxy)phenyl]benzene} sulfonamido)benzoic acid,
4-{[3-(4-butoxyphenyl)benzene]sulfonamido}-2-hydroxybenzoic acid,
4-{[3-(3,4-dimethoxyphenyl)benzene] sulfonamido}-2-hydroxybenzoic acid, 2-hydroxy-4-{[3-(6-methoxypyridin-3-yl)benzene] sulfonamido} benzoic acid, 2-hydroxy-4-{[3-(morpholin-4-yl)benzene]sulfonamido}benzoic acid,
2-hydroxy-4-(5-phenyl-2,3-dihydro-1-benzofuran-7-sulfonamido)benzoic acid,
4-{[(4-bromo-5-chloro-2-thienyl)sulfonyl]amino}-2-hydroxybenzoic acid,
4-(5-bromo-6-chloro-pyridine-3-sulfonylamino)-2-hydroxy-benzoic acid,
4-(4,5-dichloro-thiophene-2-sulfonylamino)-2-hydroxy-benzoic acid,
4-(3-bromo-thiophene-2-sulfonylamino)-2-hydroxy-benzoic acid,
4-(5-bromo-thiophene-2-sulfonylamino)-2-hydroxy-benzoic acid,
4-(4-chloro-3-nitro-benzenesulfonylamino)-2-hydroxy-benzoic acid,
4-(4-bromo-2,5-dichloro-thiophene-3-sulfonylamino)-2-hydroxy-benzoic acid,
4-(3-difluoromethoxy-benzenesulfonylamino)-2-hydroxy-benzoic acid,
2-hydroxy-4-(3-methoxy-benzenesulfonylamino)-benzoic acid,
4-[5-(benzoylamino-methyl)-thiophene-2-sulfonylamino]-2-hydroxy-benzoic acid,
4-(3-chloro-4-methyl-benzenesulfonylamino)-2-hydroxy-benzoic acid,
2-hydroxy-4-(4-methyl-3-nitro-benzenesulfonylamino)-benzoic acid,
4-(3-fluoro-benzenesulfonylamino)-2-hydroxy-benzoic acid,
4-(2,5-dichloro-thiophene-3-sulfonylamino)-2-hydroxy-benzoic acid,
2-hydroxy-4-(2,3,4-trichloro-benzenesulfonylamino)-benzoic acid,
2-hydroxy-4-(4-methyl-naphthalene-1-sulfonylamino)-benzoic acid,
4-(4-fluoro-naphthalene-1-sulfonylamino)-2-hydroxy-benzoic acid,
4-(5-dimethylamino-naphthalene-1-sulfonylamino)-2-hydroxy-benzoic acid,
4-(5-chloro-3-methyl-benzo[b]thiophene-2-sulfonylamino)-2-hydroxy-benzoic acid,
2-hydroxy-4-(3-pyridin-4-yl-benzenesulfonylamino)-benzoic acid,
4-(4′-fluoro-3′-methyl-biphenyl-3-sulfonylamino)-2-hydroxy-benzoic acid,
4-(3′-chloro-biphenyl-3-sulfonylamino)-2-hydroxy-benzoic acid,
4-(4′-carbamoyl-biphenyl-3-sulfonylamino)-2-hydroxy-benzoic acid,
4-(3′-fluoro-4′-methoxy-biphenyl-3-sulfonylamino)-2-hydroxy-benzoic acid,
4-[6-chloro-5-(4-hydroxy-phenyl)-pyridine-3-sulfonylamino]-2-hydroxy-benzoic acid, 4-[6-chloro-5-(3-hydroxy-phenyl)-pyridine-3-sulfonylamino]-2-hydroxy-benzoic acid, 4-[5-(3-amino-phenyl)-6-chloro-pyridine-3-sulfonylamino]-2-hydroxy-benzoic acid,
4-[6-chloro-5-(IH-pyrazol-4-yl)-pyridine-3-sulfonylamino]-2-hydroxy-benzoic acid,
4-[6-chloro-5-(4-fluoro-3-methyl-phenyl)-pyridine-3-sulfonylamino]-2-hydroxy-benzoic acid, 4-[6-chloro-5-(3-chloro-phenyl)-pyridine-3-sulfonylamino]-2-hydroxy-benzoic acid, 4-[6-chloro-5-(2-fluoro-3-methoxy-phenyl)-pyridine-3-sulfonylamino]-2-hydroxy benzoic acid,
4-[5-(4-carbamoyl-phenyl)-6-chloro-pyridine-3-sulfonylamino]-2-hydroxy-benzoic acid, 4-[6-chloro-5-(3-fluoro-phenyl)-pyridine-3-sulfonylamino]-2-hydroxy-benzoic acid,
4-[6-chloro-5-(3-fluoro-4-methoxy-phenyl)-pyridine-3-sulfonylamino]-2-hydroxy benzoic acid,
4-(6-chloro-5-quinolin-6-yl-pyridine-3-sulfonylamino)-2-hydroxy-benzoic acid,
4-(5-chloro-4-pyridin-3-yl-thiophene-2-sulfonylamino)-2-hydroxy-benzoic acid,
4-[5-chloro-4-(3-hydroxy-phenyl)-thiophene-2-sulfonylamino]-2-hydroxy-benzoic acid, 4-[5-chloro-4-(4-hydroxy-3-methoxy-phenyl)-thiophene-2-sulfonylamino]-2-hydroxy benzoic acid,
4-[5-chloro-4-(3-chloro-phenyl)-thiophene-2-sulfonylamino]-2-hydroxy-benzoic acid, 4-[4-(4-carbamoyl-phenyl)-5-chloro-thiophene-2-sulfonylamino]-2-hydroxy-benzoic acid, 4-[5-chloro-4-(3-fluoro-4-methoxy-phenyl)-thiophene-2-sulfonylamino]-2-hydroxy-benzoic acid,
4-[4-(4-amino-3-methoxy-phenyl)-5-chloro-thiophene-2-sulfonylamino]-2-hydroxy benzoic acid,
3′-(4-carboxy-3-hydroxy-phenylsulfamoyl)-biphenyl-2-carboxylic acid methyl ester
4-(5′-chloro-2′-methoxy-biphenyl-3-sulfonylamino)-2-hydroxy-benzoic acid,
4-(2′,5′-difluoro-biphenyl-3-sulfonylamino)-2-hydroxy-benzoic acid,
2-hydroxy-4-(2′-methoxy-biphenyl-3-sulfonylamino)-benzoic acid,
4-(2′-fluoro-3′-methoxy-biphenyl-3-sulfonylamino)-2-hydroxy-benzoic acid,
2-hydroxy-4-(2′-hydroxy-biphenyl-3-sulfonylamino)-benzoic acid,
4-(2′-amino-biphenyl-3-sulfonylamino)-2-hydroxy-benzoic acid,
4-(5′-fluoro-2′-methoxy-biphenyl-3-sulfonylamino)-2-hydroxy-benzoic acid,
4-[5-chloro-4-(5-chloro-2-methoxy-phenyl)-thiophene-2-sulfonylamino]-2-hydroxy-benzoic acid,
4-[5-chloro-4-(2,5-difluoro-phenyl)-thiophene-2-sulfonylamino]-2-hydroxy-benzoic acid, 4-[5-chloro-4-(2-methoxy-phenyl)-thiophene-2-sulfonylamino]-2-hydroxy-benzoic acid, 4-[5-chloro-4-(2-fluoro-3-methoxy-phenyl)-thiophene-2-sulfonylamino]-2-hydroxy-benzoic acid,
4-[4-(2-amino-phenyl)-5-chloro-thiophene-2-sulfonylamino]-2-hydroxy-benzoic acid,
4-[5-chloro-4-(5-fluoro-2-methoxy-phenyl)-thiophene-2-sulfonylamino]-2-hydroxy-benzoic acid, 4-[5-chloro-4-(2-hydroxy-phenyl)-thiophene-2-sulfonylamino]-2-hydroxy-benzoic acid, 4-(2,3-dichloro-benzenesulfonylamino)-2-hydroxy-benzoic acid,
4-[(3-chloro-4-fluorobenzene)sulfonamido]-2-hydroxybenzoic acid,
4-(4-bromo-2,5-difluoro-benzenesulfonylamino)-2-hydroxy-benzoic acid,
2-hydroxy-4-(toluene-3-sulfonylamino)-benzoic acid,
4-(biphenyl-4-sulfonylamino)-2-hydroxy-benzoic acid,
4-(benzo[b]thiophene-3-sulfonylamino)-2-hydroxy-benzoic acid,
4-(2,5-dichloro-4-methyl-thiophene-3-sulfonylamino)-2-hydroxy-benzoic acid,
2-hydroxy-4-(2,4,5-trichloro-thiophene-3-sulfonylamino)-benzoic acid,
4-(2-chloro-6-methyl-benzenesulfonylamino)-2-hydroxy-benzoic acid,
2-hydroxy-4-(3-trifluoromethoxy-benzenesulfonylamino)-benzoic acid,
4-(benzofuran-2-sulfonylamino)-2-hydroxy-benzoic acid,
2-hydroxy-4-(5-methyl-2-trifluoromethyl-furan-3-sulfonylamino)-benzoic acid,
4-(3-chloro-2-methyl-benzenesulfonylamino)-2-hydroxy-benzoic acid,
2-hydroxy-4-(5-isopropyl-3-methyl-benzo[b]thiophene-2-sulfonylamino)-benzoic acid, 4-[4-(2,3-dihydro-benzo furan-5-yl)-thiophene-2-sulfonylamino]-2-hydroxy-benzoic acid, 2-hydroxy-4-[3-(1-hydroxy-ethyl)-benzenesulfonylamino]-benzoic acid,
2-hydroxy-4-(3-hydroxy-benzenesulfonylamino)-benzoic acid,
2-hydroxy-4-(2-hydroxy-benzenesulfonylamino)-benzoic acid,
4-(4-chloro-phenylmethanesulfonylamino)-2-hydroxy-benzoic acid,
4-(3-bromo-phenylmethanesulfonylamino)-2-hydroxy-benzoic acid,
4-(4-bromo-phenylmethanesulfonylamino)-2-hydroxy-benzoic acid,
2-hydroxy-4-(2′-hydroxy-biphenyl-4-ylmethanesulfonylamino)-benzoic acid,
4-[4-(2,3-dihydro-benzo furan-5-yl)-phenylmethanesulfonylamino]-2-hydroxy-benzoic acid, 4-(2′,5′-difluoro-biphenyl-4-ylmethanesulfonylamino)-2-hydroxy-benzoic acid,
4-(biphenyl-4-ylmethanesulfonylamino)-2-hydroxy-benzoic acid,
4-[3-(2,3-dihydro-benzo furan-5-yl)-phenylmethanesulfonylamino]-2-hydroxy-benzoic acid,
4-(2′,5′-difluoro-biphenyl-3-ylmethanesulfonylamino)-2-hydroxy-benzoic acid,
4-(3,5-dibromo-thiophene-2-sulfonylamino)-2-hydroxy-benzoic acid,
4-(3,4-dibromo-thiophene-2-sulfonylamino)-2-hydroxy-benzoic acid,
4-(4,5-dibromo-thiophene-2-sulfonylamino)-2-hydroxy-benzoic acid,
4-(5-bromo-4-methyl-thiophene-2-sulfonylamino)-2-hydroxy-benzoic acid, 4-(5-chloro-4-methyl-thiophene-2-sulfonylamino)-2-hydroxy-benzoic acid, 4-(3,5-dichloro-benzenesulfonylamino)-2-hydroxy-benzoic acid,
4-(3-bromo-5-trifluoromethyl-benzenesulfonylamino)-2-hydroxy-benzoic acid,
4-(2′,5′-difluoro-5-trifluoromethyl-biphenyl-3-sulfonylamino)-2-hydroxy-benzoic acid, 4-[3-(2,3-dihydro-benzo furan-5-yl)-5-trifluoromethyl-benzenesulfonylamino]-2-hydroxy-benzoic acid,
2-hydroxy-4-(2′-hydroxy-5-trifluoromethyl-biphenyl-3-sulfonylamino)-benzoic acid, 4-(3-chloro-2-fluoro-benzenesulfonylamino)-2-hydroxy-benzoic acid,
4-(5-chloro-2-fluoro-benzenesulfonylamino)-2-hydroxy-benzoic acid,
4-(2,5-dimethyl-furan-3-sulfonylamino)-2-hydroxy-benzoic acid,
4-[5-(2,5-difluoro-phenyl)-thiophene-2-sulfonylamino]-2-hydroxy-benzoic acid,
4-[5-(2,3-dihydro-benzo furan-5-yl)-thiophene-2-sulfonylamino]-2-hydroxy-benzoic acid, 2-hydroxy-4-(5-phenyl-thiophene-2-sulfonylamino)-benzoic acid,
2-hydroxy-4-[5-(2-hydroxy-phenyl)-thiophene-2-sulfonylamino]-benzoic acid,
2-hydroxy-4-[(4-phenoxybenzene)sulfonamido]benzoic acid,
4-(2,5-dimethyl-thiophene-3-sulfonylamino)-2-hydroxy-benzoic acid,
4-(4-chloro-benzenesulfonylamino)-2-hydroxy-benzoic acid,
2-hydroxy-4-(3-nitro-benzenesulfonylamino)-benzoic acid,
2-hydroxy-4-(naphthalene-1-sulfonylamino)-benzoic acid,
2-hydroxy-4-(naphthalene-2-sulfonylamino)-benzoic acid,
4-(3-carboxy-benzenesulfonylamino)-2-hydroxy-benzoic acid,
4-(4-carboxy-benzenesulfonylamino)-2-hydroxy-benzoic acid,
4-(3-chloro-benzenesulfonylamino)-2-hydroxy-benzoic acid,
4-(3-bromo-5-chloro-thiophene-2-sulfonylamino)-2-hydroxy-benzoic acid, and
4-(4-bromo-thiophene-3-sulfonylamino)-2-hydroxy-benzoic acid,
or a pharmaceutically acceptable salt thereof.

8. A compound of formula (I)

wherein: n is 0 or 1;

A is 0, S, —CR⁴═CR⁴— or —CR⁴═N—;

R¹ is selected from H; halogen; C1-C6 alkyl, optionally substituted with at least one halogen; and C1-C6 alkoxy, substituted with at least one halogen; R² and R³ are each independently selected from H; halogen; C1-C6 alkyl; C1-C6 alkoxy; secondary or tertiary CI-C6 alkylamido; carbocyclylcarbonylamino-C0-C2 alkyl; 5- or 6-membered cyclic aminocarbonyl; C1-C6 alkylcarbonylamino; C1-C6 alkylsulfonyl; hydro xy-C0-C6 alkyl, C1-C6 alkylcarbonyl; carboxy; C1-C6 alkoxycarbonyl; cyano; carbocyclyloxy; heterocyclyloxy; carbocyclyl-C0-C3 alkyl; carbocyclyl-C2-C3 alkenyl; heterocyclyl-C0-C3 alkyl; and heterocyclyl-C2-C3 alkenyl; wherein any alkyl is optionally substituted with at least one halogen; any carbocyclyl or heterocyclyl is 5- or 6-membered monocyclyl or 9- or 10-membered bicyclyl; and any carbocyclyl or heterocyclyl is optionally substituted with at least one R⁵; or R² and R³ form, together with the carbon atoms to which they are attached, a 5- or 6-membered carbocyclic or heterocyclic ring, which ring is optionally substituted with at least one R⁵; each R⁴ is independently selected from H, halogen, monocyclic C3-C6 carbocyclyl and C1-C6 alkyl, wherein any alkyl is optionally substituted with at least one halogen; each R⁵ is independently selected from halogen; C1-C6 alkyl; C1-C6 alkoxy; phenoxy; amino; cyano; nitro; secondary or tertiary C1-C6 alkylamino; 5- or 6-membered cyclic amino; C1-C6 alkylcarbonylamino; carbamoyl; secondary or tertiary C1-C6 alkylamido; 5- or 6-membered cyclic aminocarbonyl; C1-C6 alkoxycarbonylamino; hydroxy-C0-C6 alkyl; C1-C6-alkylthio; carboxy-C0-C6-alkyl; C1-C6 alkoxycarbonyl; C1-C6 alkylcarbonyl; C1-C6-alkylsulfonyl; and C1-C6 alkylsulfonylamino; wherein any alkyl is optionally substituted with at least one halogen; and pharmaceutically acceptable salts thereof, with the proviso that when A is CR⁴═CR⁴ and n is 0, then neither R² nor R³ is selected from 4-hydroxypyrazolo[1,5-a]-1,3,5-triazin-8-yl and 2,4-dihydroxypyrazolo[1,5-a]-1,3,5-triazin-8-yl; and when A is —CR⁴═CR⁴—, n is 0, and R² and R³, together with the carbon atoms to which they are attached, do not form a 5- or 6-membered carbocyclic or heterocyclic ring, then:

(i) R² is in meta position relative to the sulfonamide bond;

(ii) when R¹, R² and R⁴ are all H, R³ is not selected from H; halogen; C1-C6 alkyl; C1-C6 alkoxy; C1-C6 alkylcarbonylamino; C1-C6 alkylcarbonyl; carboxy; C1-C6 alkoxycarbonyl; cyano; cyclohexyl; trifluoromethyl and trifluoromethoxy; and

(iii) when R³ is selected from H, F, Cl, Br, methyl and tert-butyl, R² is not H; and with the further proviso that the compound is not selected from
4-(5-ethylthiophene-2-sulfonamido)-2-hydroxybenzoic acid,
4-(5-bromothiophene-2-sulfonamido)-2-hydroxybenzoic acid,
4-(5-chlorothiophene-2-sulfonamido)-2-hydroxybenzoic acid,
4-(5-methylthiophene-2-sulfonamido)-2-hydroxybenzoic acid,
2-hydroxy-4-(thiophene-2-sulfonamido)benzoic acid,
4-(3-bromothiophene-2-sulfonamido)-2-hydroxybenzoic acid,
4-(2,5-dibromothiophene-3-sulfonamido)-2-hydroxybenzoic acid,
4-(2,5-dichlorothiophene-3-sulfonamido)-2-hydroxybenzoic acid,
4-(2,5-methylthiophene-3-sulfonamido)-2-hydroxybenzoic acid,
4-(4-bromo-3-carboxyphenylsulfonamido)-2-hydroxybenzoic acid,
4-(4-fluoro-3-methylphenylsulfonamido)-2-hydroxybenzoic acid,
4-(3,4-diethoxyphenylsulfonamido)-2-hydroxybenzoic acid,
4-(2,3-dihydro-1H-indene-5-sulfonamido)-2-hydroxybenzoic acid,
2-hydroxy-4-(2, 3,4,5, 6-pentamethylphenylsulfonamido)benzoic acid,
4-(3,5-dichlorophenylsulfonamido)-2-hydroxybenzoic acid,
4-(2,3-dichlorophenylsulfonamido)-2-hydroxybenzoic acid,
4-(3-chloro-4-fluorophenylsulfonamido)-2-hydroxybenzoic acid,
4-(2-chloro-5-(trifluoromethyl)phenylsulfonamido)-2-hydroxybenzoic acid,
2-hydroxy-4-(3-(trifluoromethyl)phenylsulfonamido)benzoic acid,
4-(2-bromo-4,5-dimethoxyphenylsulfonamido)-2-hydroxybenzoic acid,
4-(2,5-difluorophenylsulfonamido)-2-hydroxybenzoic acid,
4-(3,4-dimethoxyphenylsulfonamido)-2-hydroxybenzoic acid,
4-(3,4-dimethylphenylsulfonamido)-2-hydroxybenzoic acid, 4-(4-chloro-3-(trifluoromethyl)phenylsulfonamido)-2-hydroxybenzoic acid, 4-(3,4-difluorophenylsulfonamido)-2-hydroxybenzoic acid,
4-(2,3-dihydrobenzo[b][1,4]dioxine-6-sulfonamido)-2-hydroxybenzoic acid, 4-(5-bromo-2-methylphenylsulfonamido)-2-hydroxybenzoic acid,
4-(3-cyanophenylsulfonamido)-2-hydroxybenzoic acid,
4-(3-acetamido-4-methoxyphenylsulfonamido)-2-hydroxybenzoic acid, 4-(2,5-dichloro-3,6-dimethylphenylsulfonamido)-2-hydroxybenzoic acid, 2-hydroxy-4-(5,6,7,8-tetrahydronaphthalene-2-sulfonamido)benzoic acid, 4-(4-bromo-3-methylphenylsulfonamido)-2-hydroxybenzoic acid,
4-(3-acetylphenylsulfonamido)-2-hydroxybenzoic acid,
4-(3-bromophenylsulfonamido)-2-hydroxybenzoic acid,
4-(3-chlorophenylsulfonamido)-2-hydroxybenzoic acid,
2-hydroxy-4-(4-methoxy-3-(IH-tetrazol-I-yl)phenylsulfonamido)benzoic acid,
4-(3-fluorophenylsulfonamido)-2-hydroxybenzoic acid,
2-hydroxy-4-(3-methylphenylsulfonamido)benzoic acid,
4-(2,5-dibromophenylsulfonamido)-2-hydroxybenzoic acid,
4-(5-(tert-butyl)-2,3-dimethylphenylsulfonamido)-2-hydroxybenzoic acid,
2-hydroxy-4-(2,3,5,6-tetramethylphenylsulfonamido)benzoic acid,
4-(3,4-dichlorophenylsulfonamido)-2-hydroxybenzoic acid,
4-(2,5-dimethylphenylsulfonamido)-2-hydroxybenzoic acid,
4-(3-carboxyphenylsulfonamido)-2-hydroxybenzoic acid,
2-hydroxy-4-(naphthalene-2-sulfonamido)benzoic acid,
4-(2,5-dichlorophenylsulfonamido)-2-hydroxybenzoic acid,
4-(2,5-diethylphenylsulfonamido)-2-hydroxybenzoic acid,
2-hydroxy-4-(2,4,5-trimethylphenylsulfonamido)benzoic acid,
4-(3-carboxy-4-hydroxyphenylsulfonamido)-2-hydroxybenzoic acid,
2-hydroxy-4-(benzylsulfonamido)benzoic acid,
4-(8-chloronaphthalene-1-sulfonamido)-2-hydroxybenzoic acid,
2-hydroxy-4-(4-methoxynaphthalene-1-sulfonamido)benzoic acid,
2-hydroxy-4-(4-methylnaphthalene-I-sulfonamido)benzoic acid,
4-(4-bromonaphthalene-1-sulfonamido)-2-hydroxybenzoic acid,
2-hydroxy-4-(naphthalene-1-sulfonamido)benzoic acid,
2-hydroxy-4-(quinoline-8-sulfonamido)benzoic acid,
4-(2-cyanobenzylsulfonamido)-2-hydroxybenzoic acid, 4-(benzylsulfonamido)-2-hydroxybenzoic acid,
4-(5-fluoropyridine-3-sulfonamido)-2-hydroxybenzoic acid,
4-(5-bromopyridine-3-sulfonamido)-2-hydroxybenzoic acid,
4-(5-chloropyridine-3-sulfonamido)-2-hydroxybenzoic acid, and
2-hydroxy-4-(pyridine-3-sulfonamido)benzoic acid.

9. The compound according to item 8, wherein one of R² and R³ is selected from carbocyclyl-C0-C3 alkyl; carbocyclyl-C2-C3 alkenyl; heterocyclyl-C0-C3 alkyl; and heterocyclyl-C2-C3 alkenyl; wherein any carbocyclyl or heterocyclyl is 5- or 6-membered monocyclyl or 9- or 10-membered bicyclyl; and any carbocyclyl or heterocyclyl is optionally substituted with at least one R⁵; or R² and R³ form, together with the carbon atoms to which they are attached, a 5- or 6-membered carbocyclic or heterocyclic ring, which ring is optionally substituted with at least one R⁵; or a pharmaceutically acceptable salt thereof.

10. The compound according to item 9, wherein R² is phenyl, optionally substituted with at least one R⁵, or R² and R³ form, together with the carbon atoms to which they are attached, a benzene ring, optionally substituted with at least one R⁵; or a pharmaceutically acceptable salt thereof.

11. The compound according item 8, wherein A is O or S; or a pharmaceutically acceptable salt thereof.

12. The compound according item 11, wherein R² is selected from H, C1-C6 alkyl, halogen, and carbocyclyl-C0-C3 alkyl, carbocyclyl-C2-C3 alkenyl, heterocyclyl-C0-C3 alkyl, heterocyclyl-C2-C3 alkenyl; wherein any carbocyclyl or heterocyclyl is optionally substituted with at least one R⁵; and

R³ is selected from H, C1-C6 alkyl, and halogen, wherein any alkyl is optionally substituted with at least one halogen; or a pharmaceutically acceptable salt thereof.

13. The compound according to any one of the items 8-10, wherein A is CR⁴—CR⁴; or a pharmaceutically acceptable salt thereof.

14. A compound selected from
4-[(biphenyl-3-ylsulfonyl)amino]-2-hydroxybenzoic acid, 4-({[3-(5-acetyl-2-thienyl)phenyl]sulfonyl}amino)-2-hydroxybenzoic acid, 2-hydroxy-4-{[(4′-hydroxybiphenyl-3-yl)sulfonyl]amino}benzoic acid,
4-[({3-[(E)-2-(4-fluorophenyl)vinyl]phenyl} sulfonyl)amino]-2-hydroxybenzoic acid, 4-{[(3′-amino-4′-methoxybiphenyl-3-yl)sulfonyl] amino}-2-hydroxybenzoic acid,
2-hydroxy-4-{[(3-pyridin-3-ylphenyl)sulfonyl]amino}benzoic acid,
4-({[4′-(dimethylamino)biphenyl-3-yl]sulfonyl}amino)-2-hydroxybenzoic acid,
2-hydroxy-4-({[5-(trifluoromethyl)biphenyl-3-yl]sulfonyl}amino)benzoic acid,
4-{[(4,6-difluorobiphenyl-3-yl)sulfonyl]amino}-2-hydroxybenzoic acid,
2-hydroxy-4-{[(6-methoxybiphenyl-3-yl)sulfonyl]amino}benzoic acid,
4-{[(5-chloro-4-phenyl-2-thienyl)sulfonyl]amino}-2-hydroxybenzoic acid,
2-hydroxy-4-({[2′-(hydroxymethyl)biphenyl-3-yl]sulfonyl}amino)benzoic acid,
4-{[(3′-fluorobiphenyl-3-yl)sulfonyl] amino}-2-hydroxybenzoic acid,
4-{[(2′,6′-difluorobiphenyl-3-yl)sulfonyl]amino}-2-hydroxybenzoic acid,
2-hydroxy-4-({[3′-(isopropoxycarbonyl)biphenyl-3-yl]sulfonyl}amino)benzoic acid, 4-({[3-(2,3-dihydro-1-benzofuran-5-yl)phenyl]sulfonyl} amino)-2-hydroxybenzoic acid, 4-{[(3′-fluoro-4′-hydroxybiphenyl-3-yl)sulfonyl]amino}-2-hydroxybenzoic acid,
2-hydroxy-4-{[(3-quinolin-6-ylphenyl)sulfonyl]amino} benzoic acid,
4-{[(3′-aminobiphenyl-3-yl)sulfonyl]amino}-2-hydroxybenzoic acid,
2-hydroxy-4-({[3-(2-methyl-1,3-thiazol-4-yl)phenyl]sulfonyl}amino)benzoic acid, 4-({[5-chloro-4-(2,3-dihydro-1-benzofuran-5-yl)-2-thienyl]sulfonyl} amino)-2-hydroxybenzoic acid,
4-({[5-chloro-4-(3-fluoro-4-hydroxyphenyl)-2-thienyl]sulfonyl}amino)-2-hydroxybenzoic acid,
4-{[(5-chloro-4-quinolin-6-yl-2-thienyl)sulfonyl]amino}-2-hydroxybenzoic acid,
4-({[4-(1,3-benzodioxol-5-yl)-5-chloro-2-thienyl]sulfonyl}amino)-2-hydroxybenzoic acid, 4-({[5-chloro-4-(4-hydroxy-3,5-dimethylphenyl)-2-thienyl]sulfonyl}amino)-2-hydroxybenzoic acid,
4-({[5-chloro-4-(2,4-difluorophenyl)-2-thienyl]sulfonyl}amino)-2-hydroxybenzoic acid, 4-({[4-(3-acetylphenyl)-5-chloro-2-thienyl]sulfonyl}amino)-2-hydroxybenzoic acid, 4-[({5-chloro-4-[2-(hydroxymethyl)phenyl]-2-thienyl} sulfonyl)amino]-2-hydroxybenzoic acid,
4-({[5-chloro-4-(3-fluorophenyl)-2-thienyl]sulfonyl}amino)-2-hydroxybenzoic acid, 4-[({5-chloro-4-[3-(isopropoxycarbonyl)phenyl]-2-thienyl}sulfonyl)amino]-2-hydroxybenzoic acid, 4-({[5-chloro-4-(3,5-difluorophenyl)-2-thienyl]sulfonyl}amino)-2-hydroxybenzoic acid, 4-({[5-chloro-4-(6-ethoxypyridin-3-yl)-2-thienyl]sulfonyl}amino)-2-hydroxybenzoic acid, 4-({[4-(3-aminophenyl)-5-chloro-2-thienyl]sulfonyl}amino)-2-hydroxybenzoic acid, 4-({[5-chloro-4-(4-methoxyphenyl)-2-thienyl]sulfonyl}amino)-2-hydroxybenzoic acid, 4-({[4-(4-aminophenyl)-5-chloro-2-thienyl]sulfonyl}amino)-2-hydroxybenzoic acid, tri-fluoroacetate (salt)
4-({[5-chloro-4-(4-hydroxyphenyl)-2-thienyl]sulfonyl}amino)-2-hydroxybenzoic acid,
4-[({5-chloro-4-[3-(hydroxymethyl)phenyl]-2-thienyl}sulfonyl)amino]-2-hydroxybenzoic acid,
4-[({5-chloro-4-[4-(hydroxymethyl)phenyl]-2-thienyl} sulfonyl)amino]-2-hydroxybenzoic acid,
4-({[4-(3-amino-4-methoxyphenyl)-5-chloro-2-thienyl]sulfonyl}amino)-2-hydroxybenzo acid,
trifluoroacetate (salt)
4-{[(5-chloro-4-{4-[(methylsulfonyl)amino]phenyl}-2-thienyl)sulfonyl] amino}-2-hydroxybenzoic acid,
4-{[(7-chloro-3-methyl-1-benzothien-2-yl)sulfonyl]amino}-2-hydroxybenzoic acid, 4-{[(5-chloro-3-methyl-1-benzothien-2-yl)sulfonyl] amino}-2-hydroxybenzoic acid, 2-hydroxy-4-{[(3-piperidin-I-ylphenyl)sulfonyl]amino}benzoic acid,
4-{[(3-tert-butylphenyl)sulfonyl]amino}-2-hydroxybenzoic acid,
2-hydroxy-4-{[(4-phenyl-2-thienyl)sulfonyl]amino}benzoic acid,
2-hydroxy-4-[[3-(piperidine-1-carbonyl)phenyl]sulfonylamino]benzoic acid,
2-hydroxy-4-[[3-(methylcarbamoyl)phenyl] sulfonylamino]benzoic acid,
2-hydroxy-4-[[3-(4-methylsulfonylphenyl)phenyl] sulfonylamino]benzoic acid,
4-[[3-(3-ethoxyphenyl)phenyl] sulfonylamino]-2-hydroxy-benzoic acid,
4-[[3-(3-acetamidophenyl)phenyl] sulfonylamino]-2-hydroxy-benzoic acid,
4-[[3-(3,4-dichlorophenyl)phenyl]sulfonylamino]-2-hydroxy-benzoic acid,
4-[[3-(3-carbamoylphenyl)phenyl] sulfonylamino]-2-hydroxy-benzoic acid,
4-[[3-(3-cyanophenyl)phenyl] sulfonylamino]-2-hydroxy-benzoic acid,
2-hydroxy-4-[[3-(4-nitrophenyl)phenyl] sulfonylamino]benzoic acid,
2-hydroxy-4-[[3-[3-(trifluoromethyl)phenyl]phenyl] sulfonylamino]benzoic acid,
2-hydroxy-4-[[3-(4-methylsulfanylphenyl)phenyl]sulfonylamino]benzoic acid,
2-hydroxy-4-[[3-[4-(trifluoromethoxy)phenyl]phenyl]sulfonylamino]benzoic acid, 4-[[3-(2-acetylphenyl)phenyl] sulfonylamino]-2-hydroxy-benzoic acid, 2-hydroxy-4-[[3-(4-phenoxyphenyl)phenyl]sulfonylamino]benzoic acid, 2-hydroxy-4-[[3-(4-hydroxy-3-methoxy-phenyl)phenyl]sulfonylamino]benzoic acid, 2-hydroxy-4-[(3-methylsulfonylphenyl)sulfonylamino]benzoic acid,
4-[[3-(benzofuran-2-yl)phenyl] sulfonylamino]-2-hydroxy-benzoic acid,
2-hydroxy-4-[[3-[4-(methoxycarbonylamino)phenyl]phenyl] sulfonylamino]benzoic acid, 4-[(5-fluoro-2-methylbenzene)sulfonamido]-2-hydroxybenzoic acid,
4-[(2-bromo-4-iodobenzene)sulfonamido]-2-hydroxybenzoic acid,
2-hydroxy-4-[(2,4,5-trichlorobenzene)sulfonamido]benzoic acid,
2-hydroxy-4-{[4-(I 3-oxazol-5-yl)benzene]sulfonamido}benzoic acid,
4-(2, 1,3-benzothiadiazole-4-sulfonamido)-2-hydroxybenzoic acid,
4-(2, 1,3-benzoxadiazole-4-sulfonamido)-2-hydroxybenzoic acid,
4-{[3-(4-chlorophenyl)benzene]sulfonamido}-2-hydroxybenzoic acid,
2-hydroxy-4-({3-[4-(trifluoromethyl)phenyl]benzene} sulfonamido)benzoic acid, 4-{[3-(4-fluorophenyl)benzene] sulfonamido}-2-hydroxybenzoic acid,
4-{[3-(3,5-dichlorophenyl)benzene]sulfonamido}-2-hydroxybenzoic acid,
2-hydroxy-4-{[3-(4-methoxyphenyl)benzene]sulfonamido}benzoic acid,
2-hydroxy-4-{[3-(4-methylphenyl)benzene]sulfonamido}benzoic acid,
4-(1-benzothiophene-2-sulfonamido)-2-hydroxybenzoic acid,
2-hydroxy-4-(5-methyl-1-benzothiophene-2-sulfonamido)benzoic acid,
2-hydroxy-4-(7-methoxy-3-methyl-1-benzothiophene-2-sulfonamido)benzoic acid, 2-hydroxy-4-(5-methoxy-3-methyl-1-benzothiophene-2-sulfonamido)benzoic acid, 2-hydroxy-4-[3-methyl-5-(propan-2-yl)-1-benzofuran-2-sulfonamido]benzoic acid, 4-(5-fluoro-3-methyl-1-benzothiophene-2-sulfonamido)-2-hydroxybenzoic acid, 4-{[3-(2H-1,3-benzodioxol-5-yl)benzene]sulfonamido}-2-hydroxybenzoic acid,
4-{[3-(2,4-difluorophenyl)benzene]sulfonamido}-2-hydroxybenzoic acid,
2-hydroxy-4-{[3-(2-nitrophenyl)benzene]sulfonamido}benzoic acid,
2-hydroxy-4-{[3-(4-hydroxy-3,5-dimethylphenyl)benzene]sulfonamido}benzoic acid, 4-{[3-(4-butylphenyl)benzene] sulfonamido}-2-hydroxybenzoic acid,
4-({3-[4-(ethanesulfonyl)phenyl]benzene}sulfonamido)-2-hydroxybenzoic acid, 2-hydroxy-4-{[3-(4-methoxy-3-methylphenyl)benzene]sulfonamido}benzoic acid, 2-hydroxy-4-{[3-(3-hydroxyphenyl)benzene]sulfonamido} benzoic acid,
2-hydroxy-4-{[3-(3-methanesulfonylphenyl)benzene]sulfonamido} benzoic acid, 4-({3-[4-(dimethylcarbamoyl)phenyl]benzene} sulfonamido)-2-hydroxybenzoic acid, 4-{[3-(4-ethylphenyl)benzene]sulfonamido}-2-hydroxybenzoic acid, 4-[(3-{4-[bis(propan-2-yl)carbamoyl]phenyl}benzene)sulfonamido]-2-hydroxybenzoic acid, 4-{[3-(4-acetylphenyl)benzene]sulfonamido}-2-hydroxybenzoic acid,
4-{[3-(2,3-dimethoxyphenyl)benzene] sulfonamido}-2-hydroxybenzoic acid,
4-{[3-(4-fluoro-2-methoxyphenyl)benzene]sulfonamido}-2-hydroxybenzoic acid,
2-hydroxy-4-{[3-(2,3,6-trifluorophenyl)benzene]sulfonamido}benzoic acid,
4-({3-[4-(2-carboxyethyl)phenyl]benzene} sulfonamido)-2-hydroxybenzoic acid,
2-hydroxy-4-{[3-(3-methylphenyl)benzene]sulfonamido} benzoic acid,
4-{[3-(3,5-difluorophenyl)benzene] sulfonamido}-2-hydroxybenzoic acid,
2-hydroxy-4-{[3-(4-methoxy-3,5-dimethylphenyl)benzene] sulfonamido} benzoic acid, 2-hydroxy-4-{[3-(2-methylphenyl)benzene]sulfonamido}benzoic acid,
2-hydroxy-4-{[3-(2-propoxypyridin-3-yl)benzene]sulfonamido}benzoic acid,
4-{[3-(6-ethoxypyridin-3-yl)benzene]sulfonamido}-2-hydroxybenzoic acid,
2-hydroxy-4-({3-[4-(propan-2-yloxy)phenyl]benzene} sulfonamido)benzoic acid,
4-{[3-(4-butoxyphenyl)benzene]sulfonamido}-2-hydroxybenzoic acid,
4-{[3-(3,4-dimethoxyphenyl)benzene] sulfonamido}-2-hydroxybenzoic acid,
2-hydroxy-4-{[3-(6-methoxypyridin-3-yl)benzene] sulfonamido} benzoic acid,
2-hydroxy-4-{[3-(morpholin-4-yl)benzene]sulfonamido}benzoic acid,
2-hydroxy-4-(5-phenyl-2,3-dihydro-1-benzofuran-7-sulfonamido)benzoic acid,
4-{[(4-bromo-5-chloro-2-thienyl)sulfonyl]amino}-2-hydroxybenzoic acid,
4-(5-bromo-6-chloro-pyridine-3-sulfonylamino)-2-hydroxy-benzoic acid,
4-(4,5-dichloro-thiophene-2-sulfonylamino)-2-hydroxy-benzoic acid,
4-(4-bromo-2,5-dichloro-thiophene-3-sulfonylamino)-2-hydroxy-benzoic acid,
4-(3-difluoromethoxy-benzenesulfonylamino)-2-hydroxy-benzoic acid,
2-hydroxy-4-(3-methoxy-benzenesulfonylamino)-benzoic acid,
4-[5-(benzoylamino-methyl)-thiophene-2-sulfonylamino]-2-hydroxy-benzoic acid,
4-(3-chloro-4-methyl-benzenesulfonylamino)-2-hydroxy-benzoic acid,
2-hydroxy-4-(4-methyl-3-nitro-benzenesulfonylamino)-benzoic acid,
2-hydroxy-4-(2,3,4-trichloro-benzenesulfonylamino)-benzoic acid,
4-(4-fluoro-naphthalene-1-sulfonylamino)-2-hydroxy-benzoic acid,
4-(5-dimethylamino-naphthalene-1-sulfonylamino)-2-hydroxy-benzoic acid,
4-(5-chloro-3-methyl-benzo[b]thiophene-2-sulfonylamino)-2-hydroxy-benzoic acid, 2-hydroxy-4-(3-pyridin-4-yl-benzenesulfonylamino)-benzoic acid,
4-(4′-fluoro-3′-methyl-biphenyl-3-sulfonylamino)-2-hydroxy-benzoic acid,
4-(3′-chloro-biphenyl-3-sulfonylamino)-2-hydroxy-benzoic acid, 4-(4′-carbamoyl-biphenyl-3-sulfonylamino)-2-hydroxy-benzoic acid,
4-(3′-fluoro-4′-methoxy-biphenyl-3-sulfonylamino)-2-hydroxy-benzoic acid,
4-[6-chloro-5-(4-hydroxy-phenyl)-pyridine-3-sulfonylamino]-2-hydroxy-benzoic acid, 4-[6-chloro-5-(3-hydroxy-phenyl)-pyridine-3-sulfonylamino]-2-hydroxy-benzoic acid, 4-[5-(3-amino-phenyl)-6-chloro-pyridine-3-sulfonylamino]-2-hydroxy-benzoic acid,
4-[6-chloro-5-(IH-pyrazol-4-yl)-pyridine-3-sulfonylamino]-2-hydroxy-benzoic acid,
4-[6-chloro-5-(4-fluoro-3-methyl-phenyl)-pyridine-3-sulfonylamino]-2-hydroxy-benzoic acid, 4-[6-chloro-5-(3-chloro-phenyl)-pyridine-3-sulfonylamino]-2-hydroxy-benzoic acid,
4-[6-chloro-5-(2-fluoro-3-methoxy-phenyl)-pyridine-3-sulfonylamino]-2-hydroxy benzoic acid,
4-[5-(4-carbamoyl-phenyl)-6-chloro-pyridine-3-sulfonylamino]-2-hydroxy-benzoic acid, 4-[6-chloro-5-(3-fluoro-phenyl)-pyridine-3-sulfonylamino]-2-hydroxy-benzoic acid,
4-[6-chloro-5-(3-fluoro-4-methoxy-phenyl)-pyridine-3-sulfonylamino]-2-hydroxy benzoic acid,
4-(6-chloro-5-quinolin-6-yl-pyridine-3-sulfonylamino)-2-hydroxy-benzoic acid,
4-(5-chloro-4-pyridin-3-yl-thiophene-2-sulfonylamino)-2-hydroxy-benzoic acid,
4-[5-chloro-4-(3-hydroxy-phenyl)-thiophene-2-sulfonylamino]-2-hydroxy-benzoic acid, 4-[5-chloro-4-(4-hydroxy-3-methoxy-phenyl)-thiophene-2-sulfonylamino]-2-hydroxy benzoic acid,
4-[5-chloro-4-(3-chloro-phenyl)-thiophene-2-sulfonylamino]-2-hydroxy-benzoic acid, 4-[4-(4-carbamoyl-phenyl)-5-chloro-thiophene-2-sulfonylamino]-2-hydroxy-benzoic acid, 4-[5-chloro-4-(3-fluoro-4-methoxy-phenyl)-thiophene-2-sulfonylamino]-2-hydroxy-benzoic acid,
4-[4-(4-amino-3-methoxy-phenyl)-5-chloro-thiophene-2-sulfonylamino]-2-hydroxy benzoic acid,
3′-(4-carboxy-3-hydroxy-phenylsulfamoyl)-biphenyl-2-carboxylic acid methyl ester
4-(5′-chloro-2′-methoxy-biphenyl-3-sulfonylamino)-2-hydroxy-benzoic acid,
4-(2′,5′-difluoro-biphenyl-3-sulfonylamino)-2-hydroxy-benzoic acid,
2-hydroxy-4-(2′-methoxy-biphenyl-3-sulfonylamino)-benzoic acid,
4-(2′-fluoro-3′-methoxy-biphenyl-3-sulfonylamino)-2-hydroxy-benzoic acid,
2-hydroxy-4-(2′-hydroxy-biphenyl-3-sulfonylamino)-benzoic acid,
4-(2′-amino-biphenyl-3-sulfonylamino)-2-hydroxy-benzoic acid,
4-(5′-fluoro-2′-methoxy-biphenyl-3-sulfonylamino)-2-hydroxy-benzoic acid, 4-[5-chloro-4-(5-chloro-2-methoxy-phenyl)-thiophene-2-sulfonylamino]-2-hydroxy-benzoic acid,
4-[5-chloro-4-(2,5-difluoro-phenyl)-thiophene-2-sulfonylamino]-2-hydroxy-benzoic acid, 4-[5-chloro-4-(2-methoxy-phenyl)-thiophene-2-sulfonylamino]-2-hydroxy-benzoic acid, 4-[5-chloro-4-(2-fluoro-3-methoxy-phenyl)-thiophene-2-sulfonylamino]-2-hydroxy-benzoic acid,
4-[4-(2-amino-phenyl)-5-chloro-thiophene-2-sulfonylamino]-2-hydroxy-benzoic acid,
4-[5-chloro-4-(5-fluoro-2-methoxy-phenyl)-thiophene-2-sulfonylamino]-2-hydroxy-benzoic acid,
4-[5-chloro-4-(2-hydroxy-phenyl)-thiophene-2-sulfonylamino]-2-hydroxy-benzoic acid, 4-(4-bromo-2,5-difluoro-benzenesulfonylamino)-2-hydroxy-benzoic acid,
4-(biphenyl-4-sulfonylamino)-2-hydroxy-benzoic acid,
4-(benzo[b]thiophene-3-sulfonylamino)-2-hydroxy-benzoic acid,
4-(2,5-dichloro-4-methyl-thiophene-3-sulfonylamino)-2-hydroxy-benzoic acid,
2-hydroxy-4-(2,4,5-trichloro-thiophene-3-sulfonylamino)-benzoic acid,
4-(2-chloro-6-methyl-benzenesulfonylamino)-2-hydroxy-benzoic acid,
2-hydroxy-4-(3-trifiuoromethoxy-benzenesulfonylamino)-benzoic acid,
4-(benzofuran-2-sulfonylamino)-2-hydroxy-benzoic acid,
2-hydroxy-4-(5-methyl-2-trifluoromethyl-furan-3-sulfonylamino)-benzoic acid,
4-(3-chloro-2-methyl-benzenesulfonylamino)-2-hydroxy-benzoic acid,
2-hydroxy-4-(5-isopropyl-3-methyl-benzo[b]thiophene-2-sulfonylamino)-benzoic acid, 4-[4-(2,3-dihydro-benzo furan-5-yl)-thiophene-2-sulfonylamino]-2-hydroxy-benzoic acid, 2-hydroxy-4-[3-(1-hydroxy-ethyl)-benzenesulfonylamino]-benzoic acid,
2-hydroxy-4-(3-hydroxy-benzenesulfonylamino)-benzoic acid,
2-hydroxy-4-(2-hydroxy-benzenesulfonylamino)-benzoic acid,
4-(4-chloro-phenylmethanesulfonylamino)-2-hydroxy-benzoic acid,
4-(3-bromo-phenylmethanesulfonylamino)-2-hydroxy-benzoic acid,
4-(4-bromo-phenylmethanesulfonylamino)-2-hydroxy-benzoic acid,
2-hydroxy-4-(2′-hydroxy-biphenyl-4-ylmethanesulfonylamino)-benzoic acid,
4-[4-(2,3-dihydro-benzo furan-5-yl)-phenylmethanesulfonylamino]-2-hydroxy-benzoic acid, 4-(2′,5′-difluoro-biphenyl-4-ylmethanesulfonylamino)-2-hydroxy-benzoic acid,
4-(biphenyl-4-ylmethanesulfonylamino)-2-hydroxy-benzoic acid,
4-[3-(2,3-dihydro-benzo furan-5-yl)-phenylmethanesulfonylamino]-2-hydroxy-benzoic acid, 4-(2′,5′-difluoro-biphenyl-3-ylmethanesulfonylamino)-2-hydroxy-benzoic acid, 4-(3,5-dibromo-thiophene-2-sulfonylamino)-2-hydroxy-benzoic acid,
4-(3,4-dibromo-thiophene-2-sulfonylamino)-2-hydroxy-benzoic acid,
4-(4,5-dibromo-thiophene-2-sulfonylamino)-2-hydroxy-benzoic acid,
4-(5-bromo-4-methyl-thiophene-2-sulfonylamino)-2-hydroxy-benzoic acid,
4-(5-chloro-4-methyl-thiophene-2-sulfonylamino)-2-hydroxy-benzoic acid,
4-(3-bromo-5-trifluoromethyl-benzenesulfonylamino)-2-hydroxy-benzoic acid,
4-(2′,5′-difluoro-5-trifluoromethyl-biphenyl-3-sulfonylamino)-2-hydroxy-benzoic acid, 4-[3-(2,3-dihydro-benzo furan-5-yl)-5-trifluoromethyl-benzenesulfonylamino]-2-hydroxy-benzoic acid,
2-hydroxy-4-(2′-hydroxy-5-trifluoromethyl-biphenyl-3-sulfonylamino)-benzoic acid, 4-(3-chloro-2-fluoro-benzenesulfonylamino)-2-hydroxy-benzoic acid,
4-(5-chloro-2-fluoro-benzenesulfonylamino)-2-hydroxy-benzoic acid,
4-(2,5-dimethyl-furan-3-sulfonylamino)-2-hydroxy-benzoic acid,
4-[5-(2,5-difluoro-phenyl)-thiophene-2-sulfonylamino]-2-hydroxy-benzoic acid,
4-[5-(2,3-dihydro-benzofuran-5-yl)-thiophene-2-sulfonylamino]-2-hydroxy-benzoic acid, 2-hydroxy-4-(5-phenyl-thiophene-2-sulfonylamino)-benzoic acid,
2-hydroxy-4-[5-(2-hydroxy-phenyl)-thiophene-2-sulfonylamino]-benzoic acid,
2-hydroxy-4-[(4-phenoxybenzene)sulfonamido]benzoic acid,
2-hydroxy-4-(3-nitro-benzenesulfonylamino)-benzoic acid,
4-(4-carboxy-benzenesulfonylamino)-2-hydroxy-benzoic acid,
4-(3-bromo-5-chloro-thiophene-2-sulfonylamino)-2-hydroxy-benzoic acid,
4-(4-bromo-thiophene-3-sulfonylamino)-2-hydroxy-benzoic acid, or a pharmaceutically acceptable salt thereof.

15. A pharmaceutical composition comprising a compound according to any one of the items 1-14 and optionally at least one pharmaceutically acceptable excipient.

Item D

PFKFB3 inhibitor for use in neuroprotection, wherein PFKFB3 inhibitor, wherein PFKFB3 is selected from those described in WO2012149528A1 incorporated here by reference.

PFKFB3 inhibitor for use in neuroprotection, wherein PFKFB3 inhibitor is selected from the following

1. A compound of formula (1)

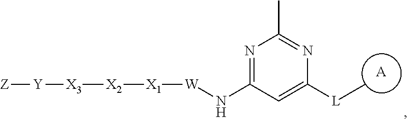

or a pharmaceutically acceptable salt thereof, wherein: W is a branched or straight C₁₋₁₂ aliphatic chain wherein up to two carbon units are optionally and independently replaced by —C(Q₁)₂-, —C(Q₂)₂-, -CHQ₁-, -CHQ₂-, —CO—, —CS—, —CONR^{A}—, —CONR^{A}NR^{A}—, —CO₂—, —OCO—, —NR^{A}—, —NR^{A}CO₂—, —O—, —NR^{A}CONR^{A}—, —OCONR^{A}—, —NR^{A}NR^{A}—, —NR^{A}CO—, —S—, —SO—, —SO₂—, —SO₂NR^{A}—, —NR^{A}SO₂—, or —NR^{A}SO₂NR^{A};

each R^{A} is independently hydrogen C₁₋₈ aliphatic; cycloaliphatic, heterocycloaliphatic, aryl, or heteroaryl, optionally substituted with 1-3 of Q₁ or Q₂;

X₁, X², and X₃ are each independently absent or are a cycloaliphatic, heterocycloaliphatic, aryl, or heteroaryl, each or which are optionally and independently substituted with 1-3 of Q₁, or Q₂, and wherein at least one of X₁, X² and X₃ is present;

Y is absent or is a branched or straight C₁₋₁₂ aliphatic chain wherein up to two carbon units are optionally and independently replaced by —C(Q₁)₂-, —C(Q₂)₂-, -CHQ₁-, -CHQ₂-, —CO—, —CS—, —CONR^{B}—, —C(═NR^{B})NR^{B}., —C(═NOR^{B})NR^{B}—, —NR^{B}C(═NR^{B})NR^{B}—, —CONR^{B}NR^{B}—, —CO₂—, —OCO—, —NR—, —NR^{B}C₀₂—, —O—, —NR^{B}CONR^{B}., —OCONR^{B}—, —NR^{B}NR^{B}—, —NR^{B}CO—, —S—, —SO—, —SO₂—, —SO₂NR^{B}—, —NRSO₂—, or —NR^{B}SO₂NR;

each R^{B} is independently hydrogen, C₁₋₈ aliphatic, cycloaliphatic, heterocycloaliphatic, aryl, or heteroaryl, optionally substituted with 1-3 of Q₁ or Q₂;

Z is independently hydrogen, C₁₋₈ aliphatic, cycloaliphatic, heterocycloaliphatic, aryl, or heteroaryl, optionally substituted with 1-3 of Q₁ or Q₂; or

L is absent or is NH, N(C₁₋₈ aliphatic), or is a branched or straight C aliphatic chain wherein up to two carbon units of L are optionally and independently replaced by —C(Q₁)₂-, —C(Q₂)₂-, —CO—, —CS—, —CONR_{C}—, —CONR^{C}NR^{C}—, —CO₂—, —OCO—, —NR^{C}—, —NR^{C}CO₂—, —O—, —NR^{C}CONR^{C}—, —OCONR^{C}—, —NR^{C}NR^{C}—, —NR^{C}CO—, —S—, —SO—, —SO₂—, —SO₂NR^{C}—, —NR^{C}SO₂—, or —NR^{C}SO₂NR^{C};

each R_{C} is independently hydrogen, C₁₋₈ aliphatic, cycloaliphatic, heterocycloaliphatic, aryl, or heteroaryl, optionally substituted with 1-3 of Q₁ or Q₂;

Ring A is a monocyclic, bicyclic, or tricyclic cycloaliphatic, heterocycloaliphatic, aryl, or heteroaryl, any of which may be optionally substituted with 1-3 of halo, —OH, oxo, —CF₃, —OCF₃, cyano, or a C₁₋₈ branched or straight aliphatic, wherein 1-3 methylene groups of the aliphatic are optionally and independently replaced with —C(O)—, —O—, —NH—, —C(O)NH—, or —C(O)O—, and wherein the aliphatic is optionally further substituted with 1-3 of halo, cyano, OH or C₁₋₃ aliphatic;

each Q, is independently halo, oxo, —CN, —NO₂, —N═O, —NHOQ₂, =NQ₂, =NOQ₂, —OQ₂, —SOQ₂, —SO₂Q₂, —SON(Q₂)₂, —SO₂(Q₂)₂, —N(Q₂)₂, —C(O)OQ₂, —C(O)-Q₂, —C(O)N(Q₂)₂, —C(═NQ₂)NQ₂-, —NQ₂C(═NQ₂)NQ₂-, —C(O)N(Q₂)(OQ₂), —N(Q₂)C(O)-Q₂, —N(Q₂)C(O)N(Q₂)₂, —N(Q₂)C(O)O-Q₂, —N(Q₂)SO₂-Q₂, —N(Q₂)SO-Q₂ or aliphatic optionally including 1-3 substituents independently selected from Q₂ or Q₃,

each Q₂ is independently hydrogen, aliphatic, alkoxy, cycloaliphatic, aryl, arylalkyl, heterocyclic, or heteroaryl ring, each optionally including 1-3 substituents independently selected from Q₃;

each Q₃ is halo, oxo, CN, NO₂, NH₂, CF₃OCF₃, OH, —COOH, or C₁-C₄ alkyl optionally substituted with 1-3 of halo, oxo, —CN, —NO₂, —CF₃, —OCF₃, —OH, —SH, —S(O)₃H, —NH₂, or —COOH;

provided that the compound of formula I is not

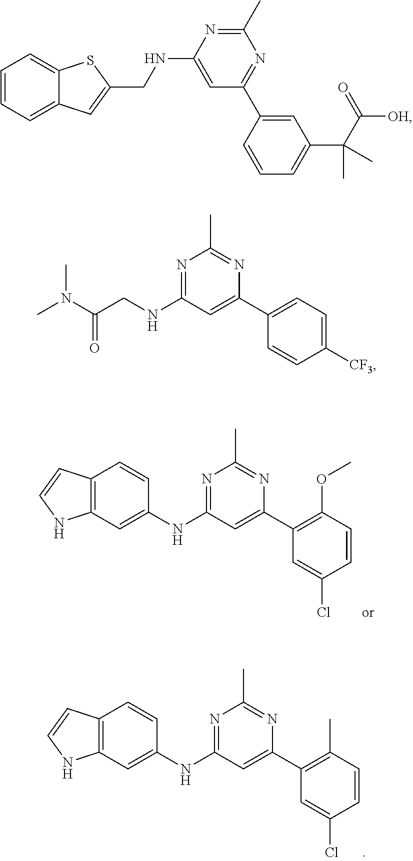 [SMILES: Cc1nc(NCc2cc3ccccc3s2)cc(-c2cccc(C(C)(C)C(=O)O)c2)n1 ; Cc1nc(NCC(=O)N(C)C)cc(-c2ccc(C(F)(F)F)cc2)n1 ; COc1ccc(Cl)cc1-c1cc(Nc2ccc3cc[nH]c3c2)nc(C)n1 ; Cc1nc(Nc2ccc3cc[nH]c3c2)cc(-c2cc(Cl)ccc2C)n1]

(i) The compound of item 1, wherein L is absent or is NH.

3. The compound of items 1-2, wherein W is absent.

4. The compound of items 1-2, wherein W is a branched or straight CM₂aliphatic chain.

5. The compound of items 4, wherein W is —CH(CH₃)—.

6. The compound of items 1-5. wherein X| is a fused bicyclic cycloaliphatic, heterocycloaliphatic, aryl, or heteroaryl and X² and X₃ are absent.

7. The compound of item 6, wherein X| is naphthalenyl, chromanyl, isochromanyl, thiocromanyl, isothiocromanyl, tetrahydroquinolinyl, tetrahydroisoquinolinyl,

tetrahydronaphthyl, indanyl, or indenyl.

8. The compound of items 1-5, wherein X | is an optionally substituted monocyclic cycloaliphatic, heterocycloaliphatic, aryl or heteroaryl.

9. The compound of item 8. wherein | is an optionally substituted cyclohexyl, oxazolyl, phenyl, pyridyl, pyrimidinyl, piperidinyl, or pyrrolidinyl.

10. The compound of item 8, wherein X| is optionally substituted phenyl, pyridyl, or pyrimidinyl.

11. The compound of items 8-10, wherein X² and X₃ are absent.

12. The compound of items 8-10, wherein X₃ is absent.

13. The compound of item 12, wherein X² is heterocycloaliphatic, aryl, or heteroaryl.

14. The compound of item 12, wherein X² is pyridyl, pyrimidinyl, tetrazolyl, triazolyl, imidazolyl, or pyrazolyl.

15. The compound of item 12. wherein X² is heterocycloaliphatic, aryl or heteroaryl and X is heterocycloaliphatic or heteroaryl.

16. The compound of items 8-10, wherein X² is pyridyl, pyrimidinyl, tetrazolyl, triazolyl, imidazolyl, or pyrazolyl and X₃ is piperizinyl, piperidinyl, or morpholinyl.

17. The compound of items 1-16 wherein;

Y is absent or is a branched or straight d.i₂ aliphatic chain wherein up to two carbon units are optionally and independently replaced by —C(Q |)₂-, —C(Q₂)₂-, -CHQ, —CHQ₂-, —CO—, —CS—, —CONR¹⁵—, —CON∥′W—, —CO₂—, —OCO—. —NR^{B}—, —NR^{B}C0₂—, -0-, —NR¹⁵C0NR^{B}—, -0C0NR^{B}—, —NR^{B}CO—: —S—, —SO—, —S0₂—, —S0₂NR—, or —NR^{B}S0₂NR^{B}; and

Z is hydrogen, C|_s aliphatic, cycloaliphatic, heterocycloaliphatic, aryl, or heteroaryl, optionally substituted with 1-3 of Qj or Q₂.

18. The compound of item 17, wherein Y is present and is a branched or straight C|.|₂ aliphatic chain wherein up to two carbon units are optionally and independently replaced by —C(Qi)₂-, —C(Q₂)₂-, —CI-IQ 1-, -CHQ₂-, —CO—, —CS—, —CONR—, —CONRNR^{B}—, —C₀₂—, —OCO—, —NR″—, —NR^{B}CO₂—, —O—, —NR^{B}CONR^{B}—, —OCONR^{B}—, —NR^{B}CO—, —S—, —SO—, —S0₂—, or —SO₂NR^{B}—.

19. The compound of items 1-18, which is a compound of formula IA, IB, or IC.

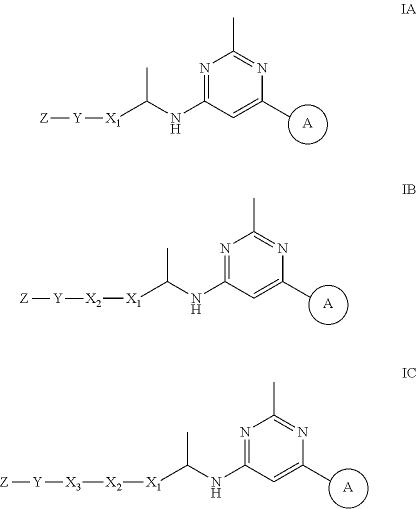

20. The compound of items 1-19, which is a compound of formula IA-1, IB-1, or IC-1.

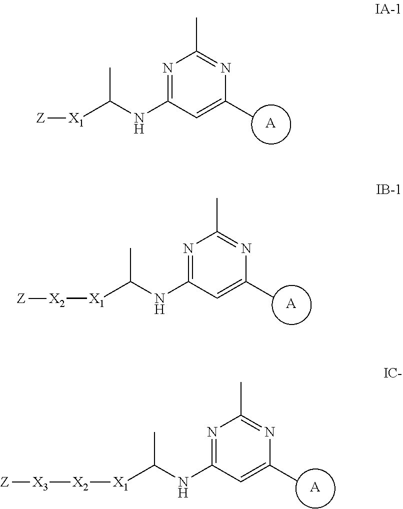

21. The compound of items 19-20, wherein i in any formula 1A or IA-1 is chosen from chromanyl, isochromanyl. thiocromanyl, isothiocromanyl, tetrahydroquinolinyl,

tetrahydroisoquinolinyl, tetrahydronaphthyl, indanyl, indenyl, each of which is optionally and independently substituted with 1-3 of halo, nitro. cyano, hydroxy, amino, Ci.₆ alkyl, C|.6 alkoxy, C|.f, alkyl, C\.(, alkylcarbonyl, Cj.6 alkoxycarbonyl, Cj-e alkylaminocarbonyl, C|.6 alkylcarbonylamino. or C i-6 alkylcarbonyloxy.

22. The compound of items 19-20. wherein i in formula IA or IA-1 is optionally substituted phenyl or pyridyl.

23. The compound of item 22. which is a compound of formula IA-2 or IB-2

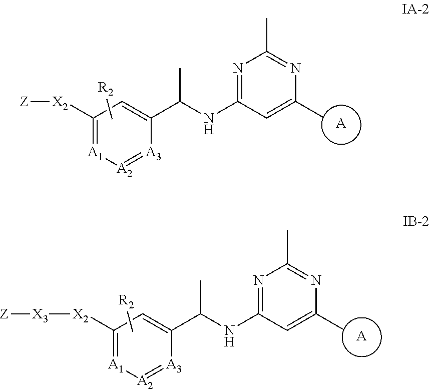

wherein:

A], and A3 are N, and A2, is CH or C-halo; or A2 is N and A | and A₃ are CH or C— halo; and

R² is H, alkyl, alkoxy, haloalkoxy, or halo.

24. The compound of items 1-23, wherein R² is H or halo.

25. The compound of item 1 which is a compound of formula IE-1

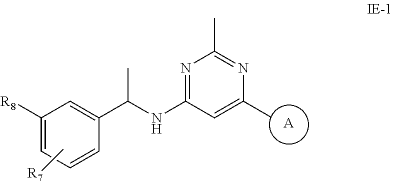

wherein R₇ is H, alkyl, haloalkyl, alkoxy, haloalkoxy, halo, OH, CN, NO₂, and R₈ is H,

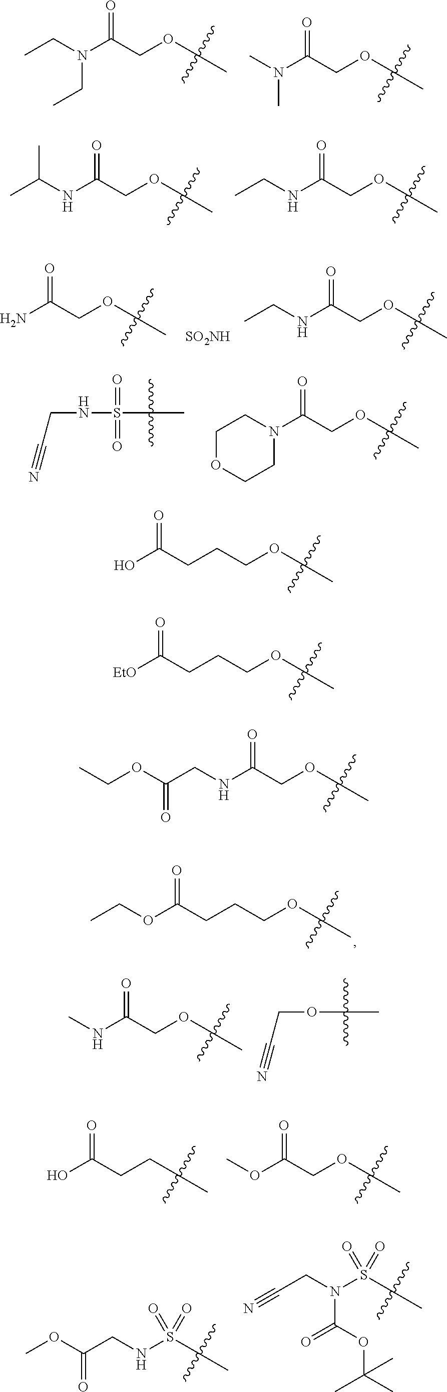

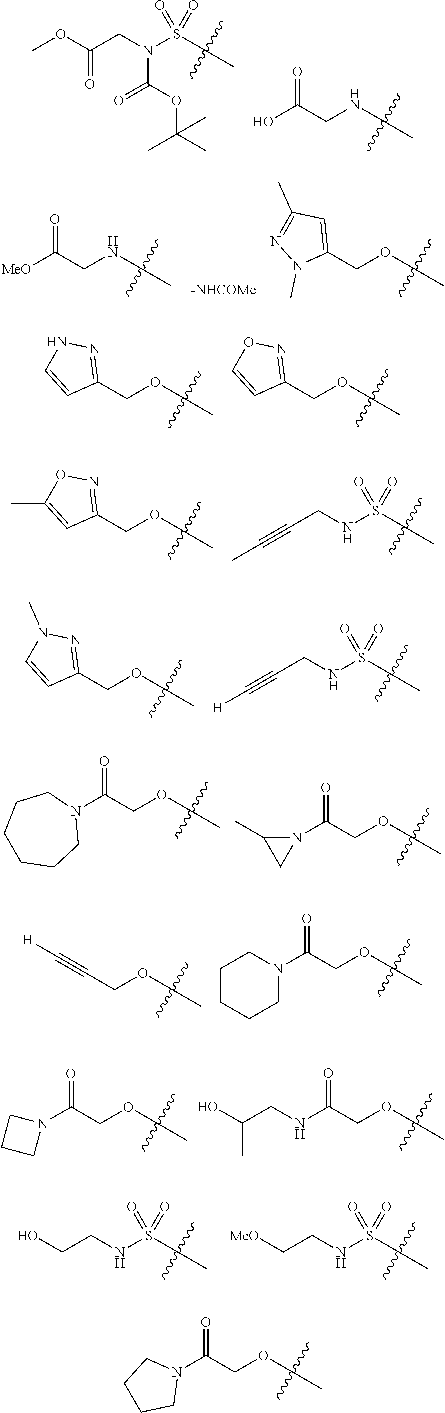

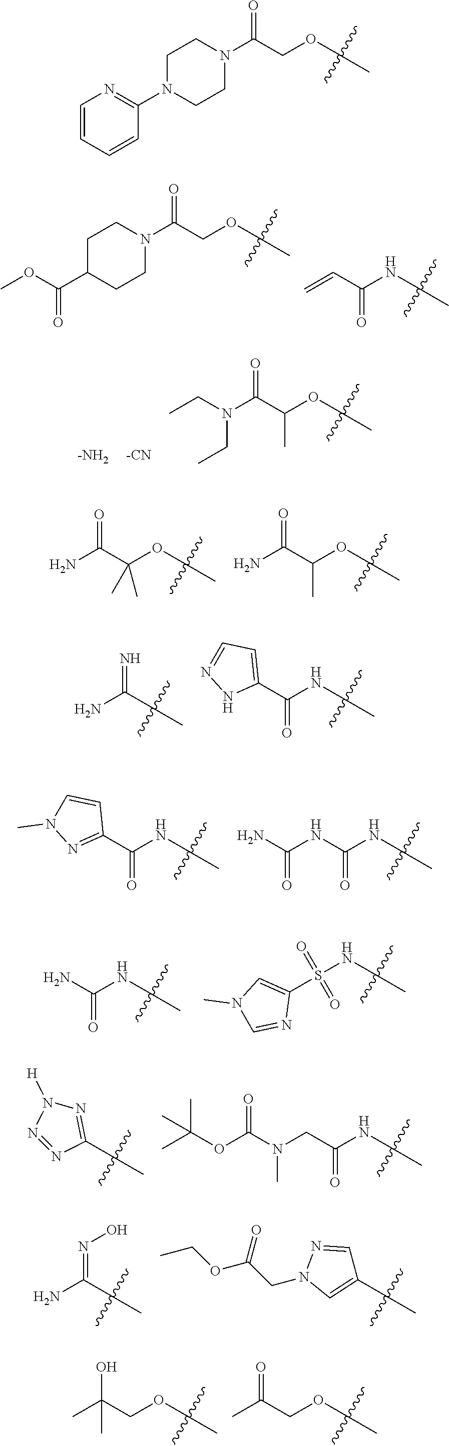

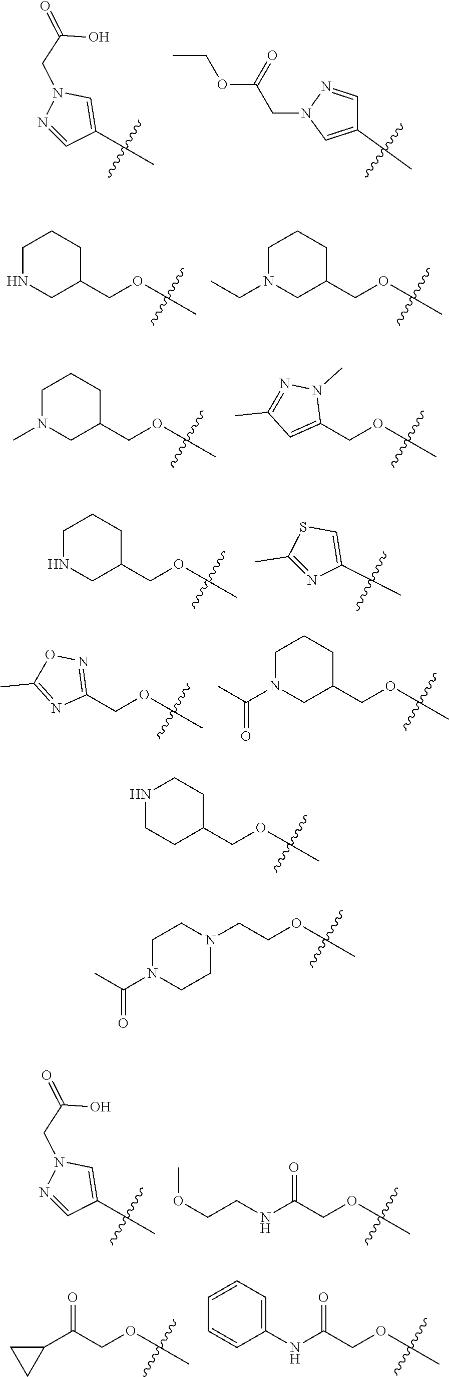

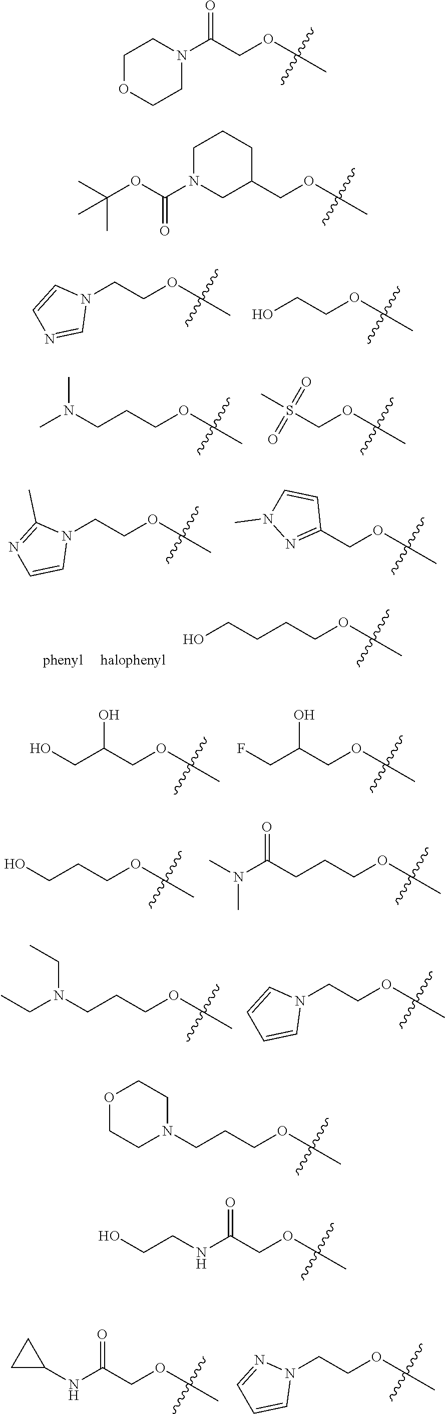

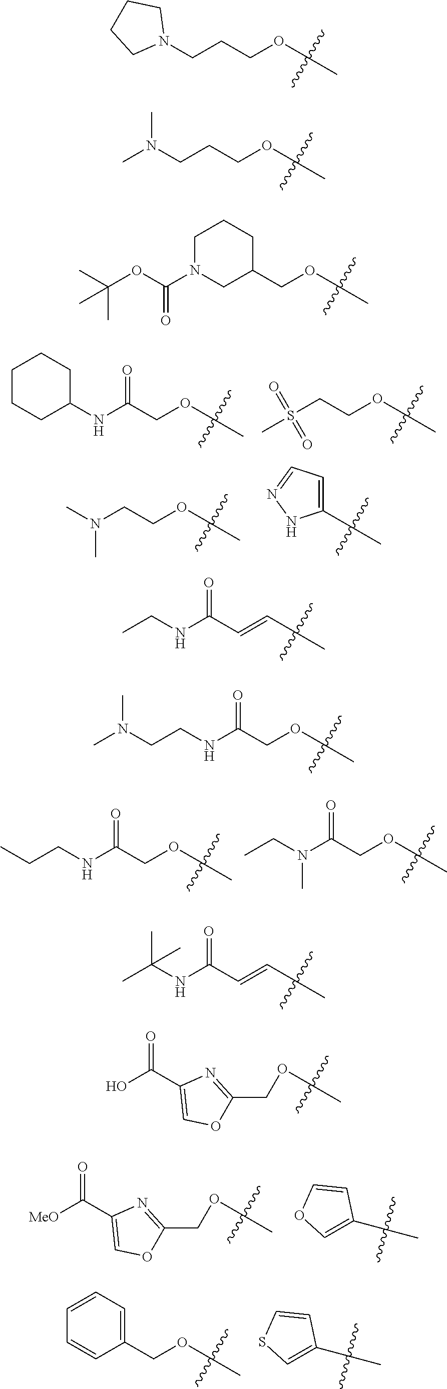

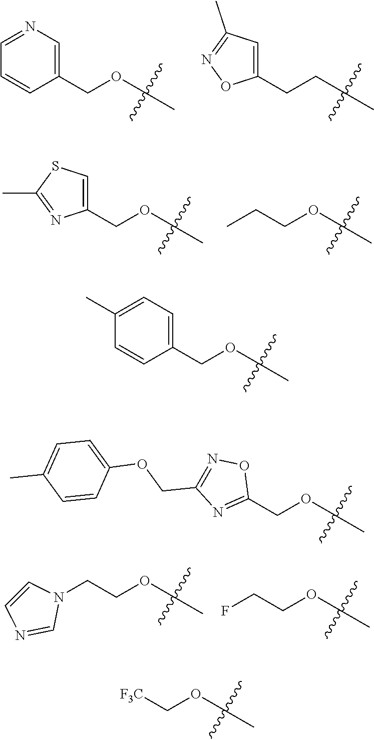

26. The compound of claim 1 which is a compound of formula IE-2

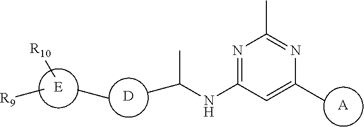

IE-2, wherein ring D is phenyl or pyridyl optionally substituted with R₇ which is H, alkyl, haloalkyl, alkoxy, haloalkoxy, halo, —OH, —CN, —NO₂; and Ring E is an aryl or heteroaryl selected from the group consisting of phenyl, pyridyl, pyrimidinyl,

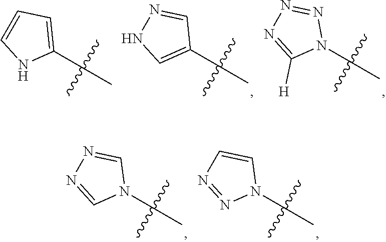 [SMILES: *c1ccc[nH]1 ; *c1cn[nH]c1 ; *n1cnnn1 ; *n1cnnc1 ; *n1ccnn1]

halo, —OH, CN, NO₂, and NH; and R₁₀ is selected from H, —CHOHCH₃, —CHOH(CH₃)₂, —COMe, —CO₂Et, —NHMe, NHEt, NMe₂, NEt₂, NHCHMe₂CH₂OH, —CH₂CO₂Et, CH₂CO₂H, CONH₂, —NHCH₂CH₂CH₂OH, —NHCH₂CO₂H, —NHCH₂CO₂Me, —NHCH₂CH₂OH, —NHCH₂CO₂H, —NHCH₂CHOHCH₂OH, —NHCH₂CH₂NH₂, —NHCH₂CH₂NMe₂, —NHCH₂CMe₂CH₂OH,

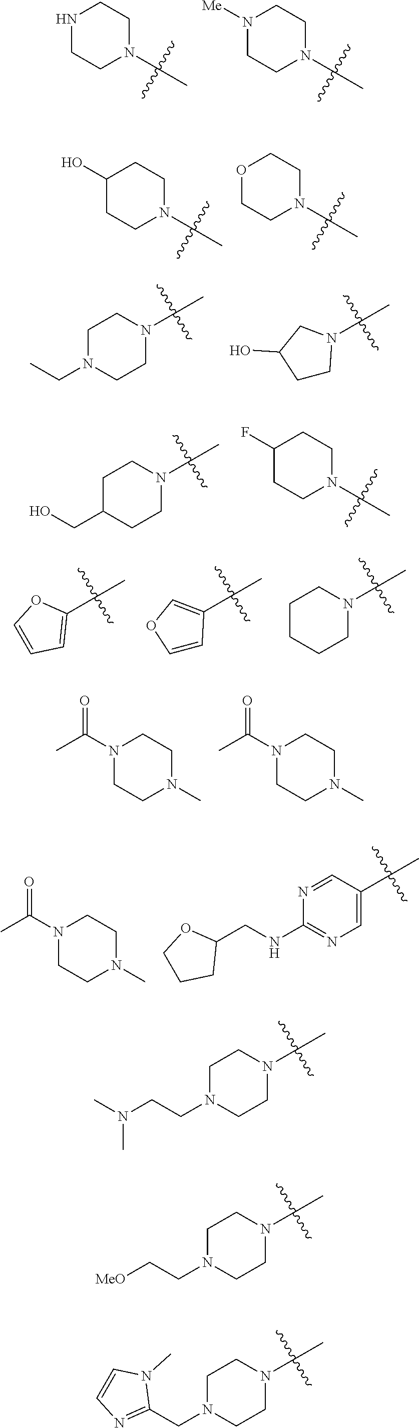

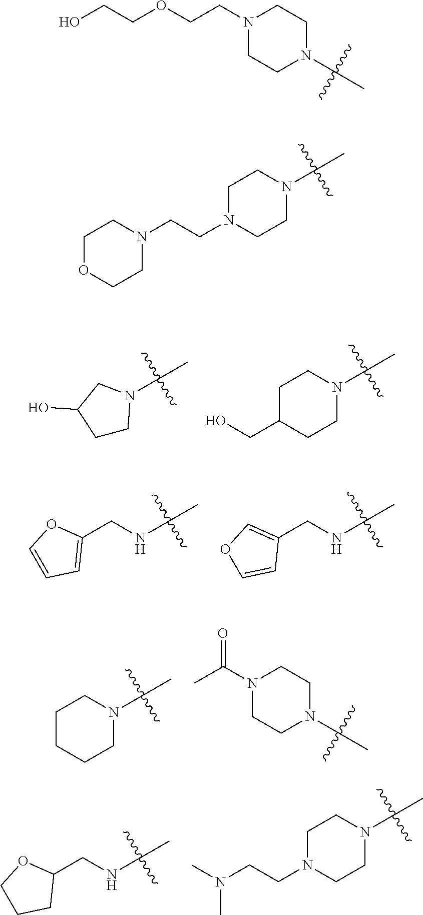

27. The compound of items 1-26 wherein Ring A is an optionally substituted monocyclic or bicyclic aryl or heteroaryl.

28. The compound of items 1-26 wherein Ring A is an optionally substituted phenyl.

29. The compound of items 1-26, wherein Ring A is

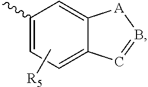

wherein at least one of A, B, and C are N, NH, N(alkyl), S, SO, SO₂, or O and the others of A, B, and C are CH, CH or C(alkyl); and R₅ is H or alkyl. In a further embodiment, one of A and C is N, S, or O. In a further embodiment, one of A and C is N, and the other of A and C is S or O.

30. The compound of claims 1-26 wherein Ring A is selected from the group consisting of phenyl substituted one, two, or three groups selected from halo, alkyl, haloalkyl. alkoxy, haloalkoxy, hydroxyalkyl. alkoxyalkyl, amino, alkylamino. dialkylamino, —CONH₂, —CONMe, —NH—CHz-CN, —CN, —CO₂alkyl, NH—CH₂—CONHMe, CH₂CONH—CH₂CH₂OH, 1,3-benzodioxol-5-yl, 2,2-difluoro-1,3-benzodioxol-5-yl, benzo[c/Jthiazol-5-yl, 1,3-benzothiazol-6-yl, 1-alkyl-1H-indol-6-yl. 1-|2-(methyloxy)ethyl]-1H-indol-6-yl, 1H-indol-4-yl, 1-methyl-H-indol-4-yl, 1H-indol-5-yl. 1H-indol-6-yl, 1-methyl-2,3-dihydro-H-indol-6-yl, 1H-indol-7-yl. 1,3-benzodioxol-5-yl. 1-benzofuran-5-yl, 3-methyl-I-benzofuran-5-yl, 7-fluoro-3-methyl-1-benzofuran-5-yl, 1-benzothien-5-yl, 1,3-benzoxazol-6-yl, 2,3-dihydro-1,4-benzodioxin-6-yl, 1H-benzimidazol-6-yl. 1-methyl-H-benzimidazol-6-yl, 1H-indazol-5-yl, 1-alkyl-1H-indazol-6-yl, 4-acetyl-3,4-dihydro-2H-I,4-benzoxazin-6-yl, 1,2,3,4-tetialiydronaphthalen-1-yl, 6-naphthalen-2-yl, and 1-methyl-H-pyrrolo[3,2-b]pyridin-6-yl.

31. The compound of claims 1-26, wherein Ring A is

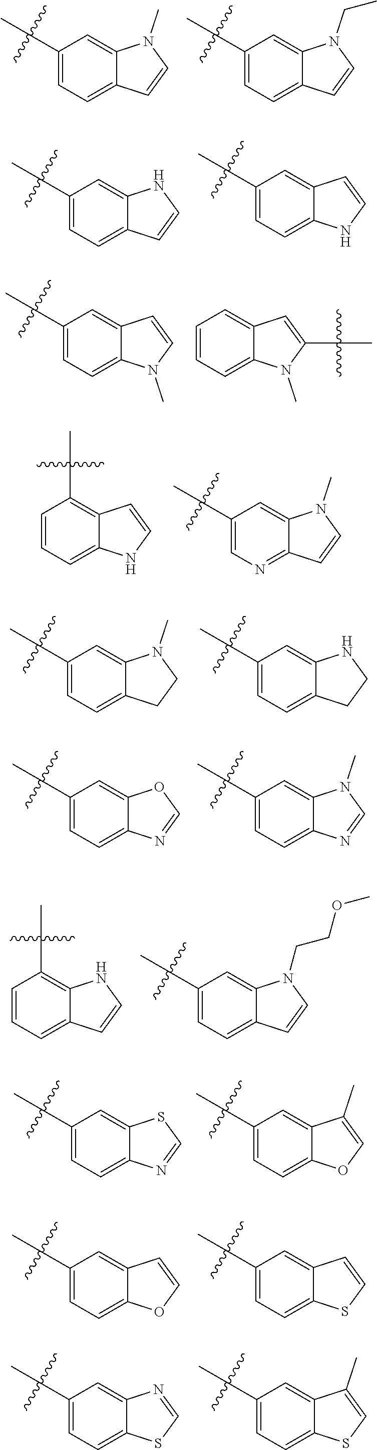

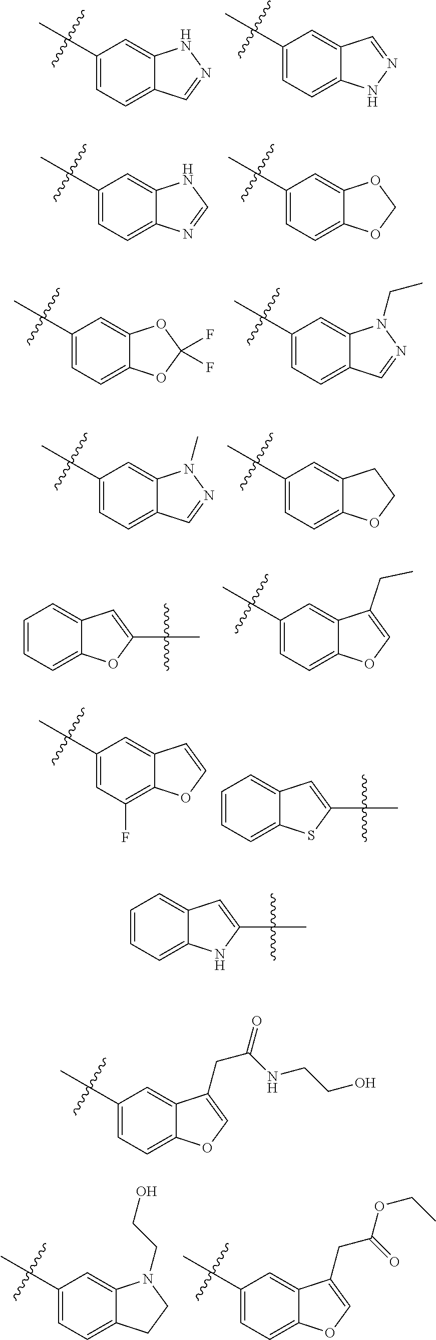

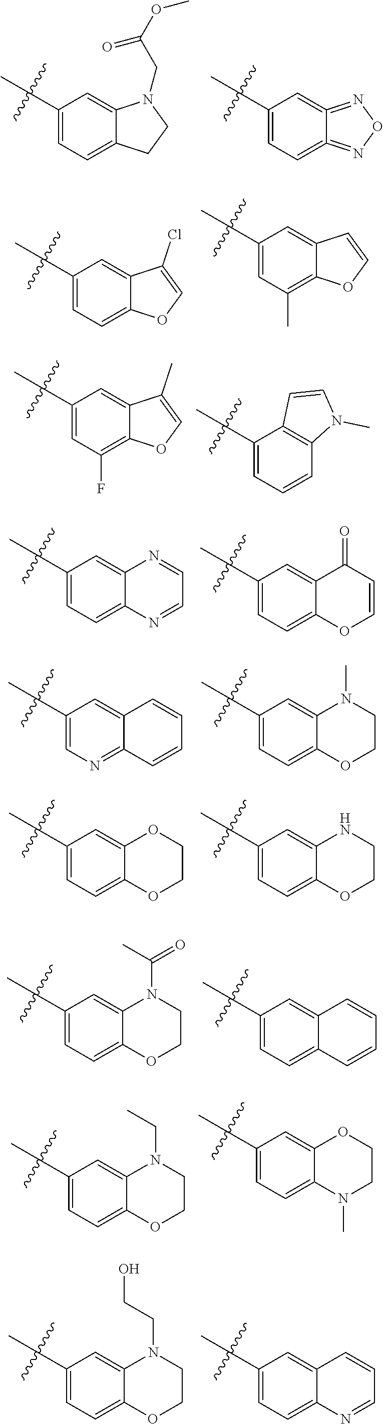

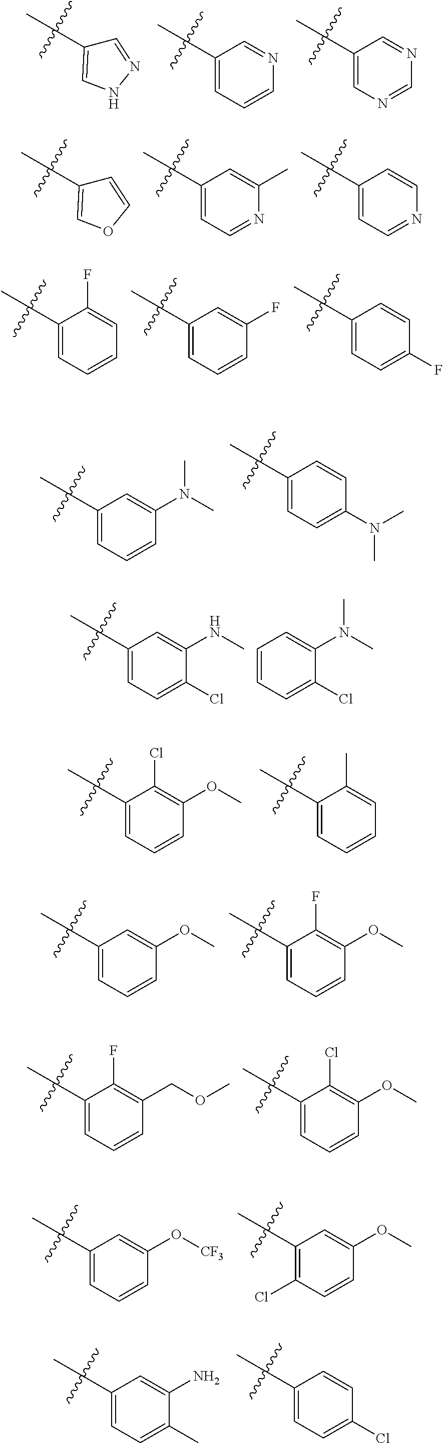

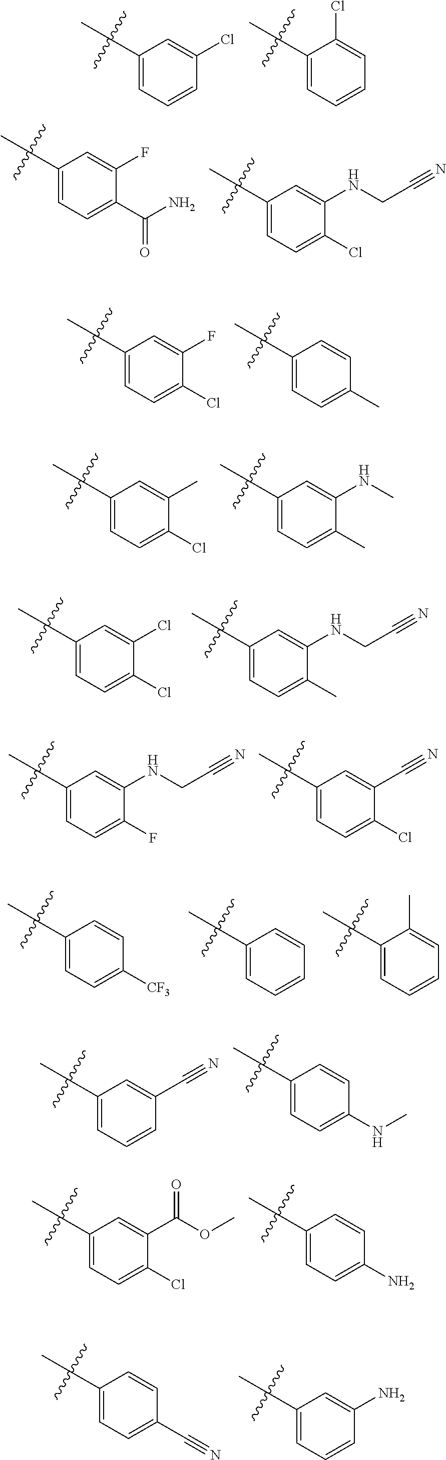

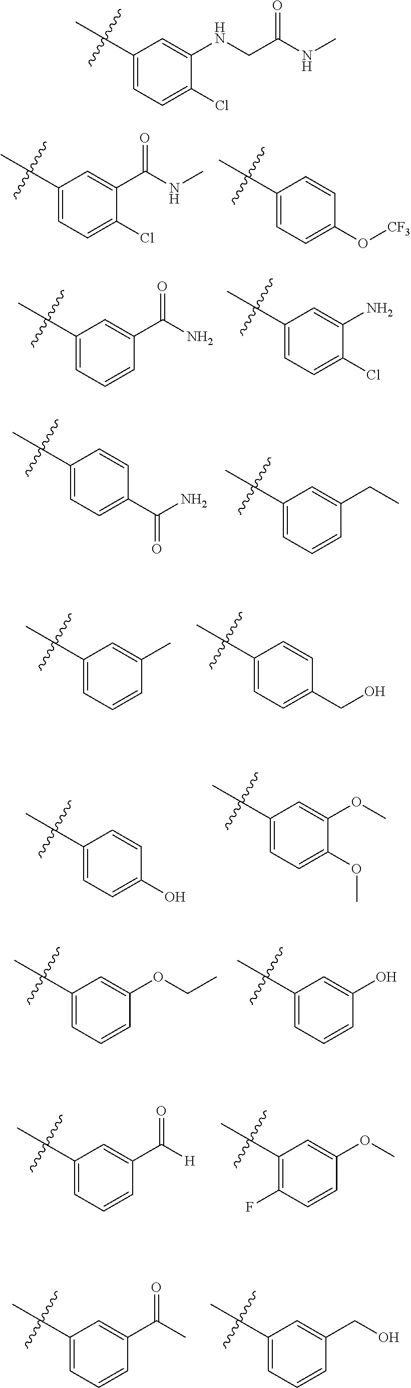

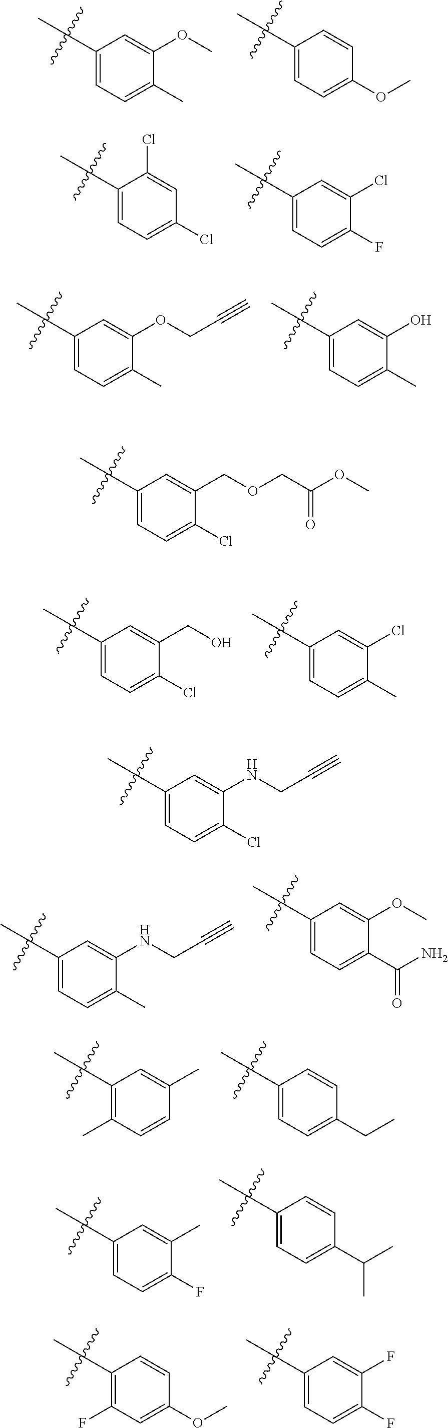

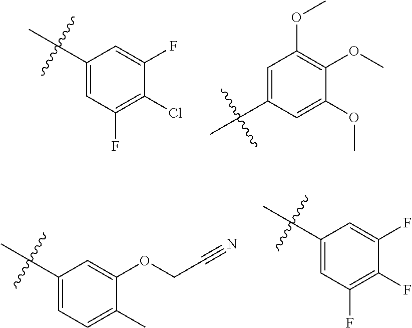 [SMILES: *c1cc(F)c(Cl)c(F)c1 ; *c1cc(OC)c(OC)c(OC)c1 ; *c1ccc(C)c(OCC#N)c1 ; *c1cc(F)c(F)c(F)c1]

32. The compound of item 1, which is a compound of formula II

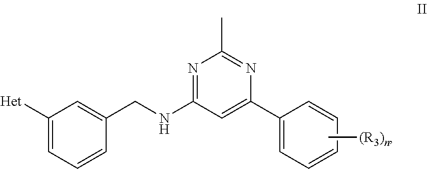

wherein: Het is a heteroaryl ring which is optionally substituted with 1-3 of Q₃; and two instances of R₃ on adjacent carbon atoms are taken together with the carbon atoms to which they are attached to form a 5-6 membered cycloaliphatic or heteroaliphatic, saturated or unsaturated ring, which is optionally substituted with 1-3 o Q₂.

33. The compound of item 31. wherein two instances of R₃ on adjacent carbon atoms are taken together with the carbon atoms to which they are attached to form a benzo-fused furanyl or thiophenyl ring, which is optionally substituted with 1-3 of Q₂.

34. The compound of item 1 which is a compound of formula HI

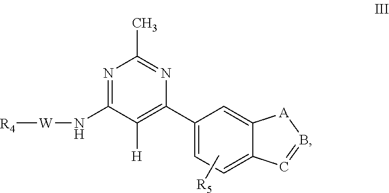

wherein: W is absent or is a branched or straight C 1.12 aliphatic chain wherein up to two carbon units are optionally and independently replaced by —C(Qi)₂-; —C(Q₂)₂-: —CHQi-, —CHQ₂-, —CO—, —CS—, —CONR^{A}—, —COR^{A}NR^{A}—, —C0₂—, —OCO—, —NR^{A}—, —NR^{A}CO₂—, —O—, —NR^{A}CONR^{A}—, —OCONR^{A}—, —NR^{A}NR′ —NR^{A}CO—, —S—, —SO—, —S0₂—, —S0₂NR^{A}—, —NR^{A}S0₂—, or

—NR^{A}S0₂NR^{A};

R is X1-X2-X3-Y—Z. wherein X|. X2. X3, Y, and Z are as previously defined;

at least one of A, B, and C are N, NH, N(alkyl), S, SO, S0₂, or 0 and the others of ABC are CH, CH, C(alkyl); and

R5 is 1-1, halo, hydroxy, alkoxy, haloalkoxy, hydroxy-alkylene, or alkyl.

35. The compound of item 34, wherein one of A and C is N, S, or O.

36. The compound of item 35. wherein one of A and C is N and the other of A and C is S.

37. The compound of item 36, which is a compound of formula IIIA, III-B, or IU-C.

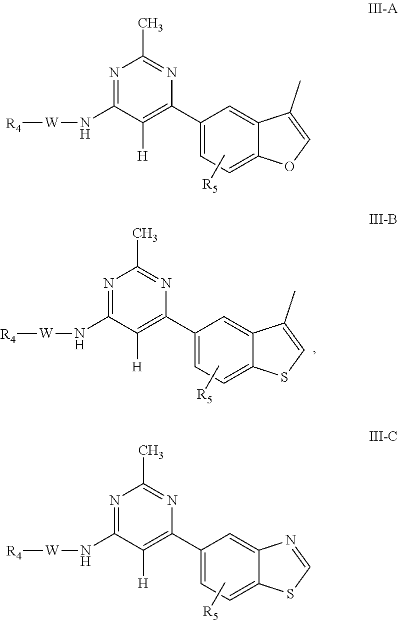

38. The compound of item 1 which is a compound of formula V

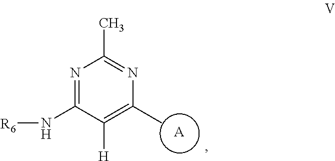

wherein Ring A is as previously defined in item 31 for a compound of formula 1 and R is cycloaliphatic, heterocycloaliphatic. aryl, or heteroaryl, each or which are optionally and independently substituted with 1-3 of Q i, Q₂, or Q₂.

39. The compound of item 38, wherein R(, is selected from the group consisting of 2,3-dihydro-1H-inden-1-yl, 1,2,3,4-tetrahydronaphthalen-1-yl. naphthalen-1-y 1, 6-nitro-3.4-dihydro-2H-chiOmen-4-yl), 3,4-dihydro-2H-chromen-6-yl, 3,4-dihydro-2H-cl romen-4-yl, 3,4-dihydro-2H-I-benzothiopyran-4-yl, furanyl, wherein each of the groups may be optionally substituted with one, two, or three groups selected from halo, nitro, —NH—CO-Me, alkyl. and alkoxy.

40. In another embodiment, the compound of formula V is a compound of formula VA.

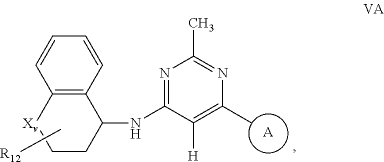

wherein Ring A is as previously defined in item 31, Xᵥ is CH₂, NH, or 0, and R|₂ is one or two groups independently selected from alkyl. —NHCOMe, —NHCOEt.

41. A compound of item which is:
(S)-5-(3-(I-(6-(7-fluoro-3-methylbenzofuran-5-yl)-2-methylpyrimidin-4-ylamino)ethyl)phenyl)pyrimidin-2-amine; (S)-2-methyl-N-(1-(5-(1-methyl-1H-pyrazol-4-yl)pyridin-3-yl)ethyl)-6-(3−inethylbenzofuran-5-yl)pyrimidin-4-amine;
(S)—N—(I-(3-(5-aminopyndin-3-yl)phenyl)ethyl)-6-(4-chloro-3-fluoi phenyl)-2-methy]pyrimidin-4-amine;
(S)-5-(3-(I-(2-methyl-6-(3-methylbenzofuran-5-yl)pyrimidin-4-ylamino)ethyl)phenyl)pyrimidin-2-amine;
N—(I-(5-(I-ethyl-IH-pyrazol-4-yl)pyridin-3-yl)ethyl)-6-(7-fluoro-3-methylbenzofuraii-5-yl)-2-methylpynmidin-4-amine;
5-(3-nuoiO-5-(1-(2-methyl-6-(3-methylbenzofuran-5-yl)pyrimidin-4-ylamino)ethyl)phenyl)pyrimidin-2-amine;
(S)-5-(3-(I-(2-methyl-6-(3-methylbenzo[b]thiophen-5-yl)pynmidin-4-ylamino)ethyl)phenyl)pyrimidin-2-amine;
(S)—N—(I-(3-(5-aminopyridin-3-yl)phenyl)ethyl)-2-metliyl-6-(3-methylbenzo[b]lhiophen-5-yl)pyrimidin-4-amine;
N-(2,3-dihydro-1H-inden-1-yl)-6-(3-II orophenyl)-2-methylpyrimidin-4-amine;
6-(Benzo[d]lhiazol-5-yl)-2-methyl-N-(2-methyibutyl)pyrimidin-4-amine;
(S)-6-(Benzo[d]thiazol-5-yl)-N—(I-cyclohexylethyl)-2-methylpyrimidin-4-amine; 2-methyl-6-[3-(methyloxy)phenyl]-N-[(1)-1,2,3,4-tetrahydronaphthalen-I-yl]pyrimidin-4-amine;
2-methyl-6-[3-(methyloxy)phenyl]-N—{(IR—)—I-[3-(methyloxy)phenyl]ethyl}pyrimidin-4-amine;
2-metlwl-6-[3-(methyloxy)phenyl]-N—{(IS)—I-[3-(melhyloxy)phenyl]ethyl}pyrimid 4-amine;
2-methyl-6-[3-(methyloxy)phenyl]-{(IS)—I-[4-(methyloxy)phenyl]ethyl}pyrimid 4-amine;
(R)-6-(Benzo[d]thiazol-5-yl)-2-methyl-N-(1,2,3,4-telrahydronaphthalen-I-yl)pyrimidin-4-amine;
,N-diethyl-2-{[3-(1-{[2-methy-6-(1-methyl-1H-indol-6-yl)pyrimidin-4-yl]amino}ethyl)phenyl]oxy}a etamide;
2-methyl-6-(I-methyl-1H-indol-6-yl)-N-[(IF)-L2₃3,4-tetrahydiOnaphlhalen-I-yrjpyrimidin-4-amine;
N-[(4R)-34-dihydiO-2H-chromen-4-yl]-2-methyl-6-(I-methyl-1H-indol-6-yl)pyrimidin-4-amine; KN-dimethyl-2-{[3-(1-{[2-methyl-6-(I-methyl-1H-indol-6-yl)pyrimidin-4-yl]amino}ethyl)phenyl]oxy}acetamide;
6-(I-ethyl-1H-indol-6-yl)-2-methyl-N-[(1R)-1,2,3,4-tetrahydronaphthalen-1-yl]pyrimidin-4-amine;
3-(I-{[2-methyl-6-(1-methyl-1H-indol-6-yl)pyrimidm-4-yl]amino}elhyl)benzenesulfonamide;
N-ethyl-2-{[3-(1-{[2-methy!-6-(1-methyl-1H-indol-6-yl)pyrimidin-4-yl]amino}ethyl)phenyl]oxy}acetamide;
N-(cyanomethyl)-3-(1-{[2-methyl-6-(1-methyl-1H-indol-6-yl)pyrimidin-4-yl]amino}ethyl)benzenesulfonamide;
2-methyl-6-(1-methyl-1H-indol-6-yl)-N-(1-{3-[(2-morpholin-4-yl-2-oxoethyl)oxy]phenyl}ethyl)pyrimidin-4-amine;
4-{[3-(1-{[2-methyl-6-(1-methyl-1H-indol-6-yl)pyrimidin-4-yl]amino}ethyl)phenyl]oxy}butanoic acid;
6-(1,3-benzodioxol-5-yl)-2-methyl-N-[(1R)-1,2,3,4-tetrahydronaphthalen-1-yl]pyrimidin-4-amine;
2-methyl-6-(1-methyl-1H-indol-6-yl)-N—{(1S)-1-[3-(methyloxy)phenyl]ethyl}pyrimidin-4-amine;
2-methyl-6-(I-methyl-IH-indol-6-yl)-N-[(IS)-I-phenylethyl]pyrimidin-4-amine; 6-(IH-indol-6-yl)-2-methyl-N-[(I I\hat{ }-I\hat{ }J\hat{ }-tclrahydronaphthalen-1-yllpyrimidin\hat{ }-amine;
6-[2-chloro-3-(methyloxy)phenyl]-2-mehyl-N—{(IS)—I-[3-(methyloxy)phenyl]ethyl}pyrimidin-4-amine;
6-(1,3-benzodioxol-5-yl)-2-methyl-N—{(IS)—I-[3-(methyloxy)phenyl]ethyl}pyrimidin-4-amine;
3-(1-{[2-methyl-6-(I-methyl-IH-indol-6-yl)pyvimidin-4-yl]amino}ethyl)phenol;
6-(1H-indol-5-yl)-2-methyl-N-[(1R)-1,2,3,4-tetrahydronaphthalen-1-yl]pyrimidin-4-amine;
6-(IH-indol-5-yl)-2-methyl-N—{(IS)—I-[3-(methyloxy)phenyl]ethyl}pyrimidin-4-amine;
ethyl N-({[3-(1-{[2-methy]-6-(I-methyl-1H-indol-6-yl)pyrimidin-4-yl]amino}ethyl)phenyl]oxy}acetyl)glycinate;
N-[(IS)—I-(4-tluorophenyl)ethyl]-2-methyl-6-(I-methyl-n-J-indol-6-yl)pyrimidin-4-amine; 2-methyl-6-(I-methyl-1H-indol-6-yl)-N-[(IS)-1-(4-methylphenyl)ethyl]pyrimidin-4-amine;
ethyl 4-{[3-(1-{[2-methy I-6-(1-methyl-1H-indol-6-yl)pyrimidin-4-yl]amino}ethyl)phenyl]oxy}butanoate;
6-(3-fluorophenyl)-2-methyl-N—{(IS)—I-[3-(methyloxy)phenyl]ethyl}pyrimidin-4-amine;
6-[2-chloro-3-(methyloxy)phenyl]-2-methyl-N-[(IR)-1,2,3,4-tetrahydronaphthaleri-yl]pyrimidin-4-amine;
2-({3-[I-({6-[2-chloro-3-(methyloxy)phenyl]-2-melhylpyriniidin-4-yl}amino)ethyl]phenyl}oxy)-N-methylacetamide;
2-{13-(I-{[6-(1,3-benzodioxol-5-yl)-2-methylpyrimidin-4-yl]amino}ethyl)phenyl]oxy}-methylacetamide;
N-(cyanomethyl)-3-[I-({2-methyl-6-[3-(methyloxy)phenyl]pyrimidin-4-yl}amino)ethyl]benzenesulfonamide;
({3-[I-({2-methyl-6-[3-(methyloxy)phenyl]pynmidin-4-yl}amino)ethyl]phenyl}oxy)acetOnitrile;
3-[1-({6-[2-chloiO-3-(melhyloxy)phenyl]-2-methylpynmidin-4-yl}amino)ethyl]benzenesulfonamide;
6-[2-n oro-3-(methyloxy)phenyl]-2-melliyl-N—{(IS)—I-[3-(mclhyloxy)phenyl]ethyl}pyrimidin-4-amine;
6-[2-chloro-3-(methyloxy)phenyl]-N-(2,3-dihydro-1H-inden-1-yl)-2-methylpyrimidin-4-amine;
N-methyl-2-({3-[I-({2-methyl-6-[3-(methyloxy)phenyl]pyrimidin-4-yl}amino)ethyl]phenyl}oxy)acetamide;
N-[I-(3-bromophenyl)ethyl]-6-[2-chloro-3-(methyloxy)phenyl]-2-methylpyrimidin-4-amine;
2-methyl-6-{I-[2-(methyloxy)ethyl]-1H-indol-6-yl}-[(IR)-1,2,3,4-telrahydiOnaphthalen-yl]pyrimidin-4-amine;
6-[2-nuoro-3-(methyloxy)phenyl]-2-methyl-N-[(IR)-1,2,3,4-tetrahydronaphthalen-I-yl]pyrimidin-4-amine;
methyl ({3-[I-({2-methyl-6-[3-(methyloxy)phenyl]pyrimidin-4-yl}amino)ethyl]phenyl}oxy)acetate;
methyl N-{3-[I-({2-methyl-6-[3-(methyloxy)phenyl]pyrimidin-4-yl}amino)ethyl]pheny 1} glycinate; -[(]S)-1-(4-chlorophenyl)ethyl]-2-methyl-6-(1-methyl-1H-indol-6-yl)pyrimidin-4-amine;
3-[I-({2-methyl-6-[13-(methyloxy)phenyl]pyrimiclin-4-yl}amino)ethyl]benzenesulfbnamide;
6-(I-ethyl-1H-indol-6-yl)-2-melhyl-N—{(IS)—I-[3-(methyloxy)phenyl]ethyl}pyriniidin-4-amine;
6-[2-chloi-5-(methyloxy)phenyl]-2-methyl-N-[(IR)-1:2,3,4-tetrahydronaphthalen-I-yl]pyrimidin-4-amine;
methyl N-({3-[1-({2-methyl-6-[3-(methyloxy)phenyl]pyrimidin-4-yl}amino)ethyrjphenyl}sulfonyl)glycinate;
2-methyl-6-(I-melhyl-IH-indol-5-yl)-N—{(IS)—I-[3-(methyloxy)phenyl]ethyl}pyrimidin-4-amine;
3-[1-({2-methyl-6-[3-(methyloxy)phenyl]pyriniidin-4-yl}amino)ethyl]phenol;
1,1-dimethylethyl (cyanomethyl)({3-[I-({2-methyl-6-[3-(methyloxy)phenyl]pyrimiclin-4-yl}amino)ethyl]phenyl}sulfonyl)carbamate;
2-methyl-6-[3-(methyloxy)phenyl]-N-{(1S)-1-[3-(trifluoromethyl)phenyl]ethyl}pyrimidin-4-ainine;
2-methyl-N—{(IS)—I-[3-(methyloxy)phenyl]ethyl}-6-{3-[(InfluoiOmelhyl)oxy]phenyl}pyrimidin-4-amine;
2-methyl-6-{3-[(methyloxy)methyl]phenyl}-N-[(IR)-1;2,34-tetrahydronaphthaleri-I-yl]pyrimidin-4-amine;
2-methyl-N—{(IS)—I-[3-(methyloxy)phenyl]ethyr}-6-[3,4,5-tris(methyloxy)phenyl]pyiimidin-4-amine;
methyl N-{[(I,I-dimethylethyl)oxy]carbonyl}-N-({3-[I-({2-methyl-6-[3-(methyloxy)phenyl]pyrimidin-4-yl}amino)ethyl]phenyl}sulfonyl)glycinate;
6-[2-chloro-5-(methyloxy)phenyl]-2-methyl-N-{(1S)—I-[3-(methyloxy)phenyl]ethyl}pyrimidin-4-amine;
2-methyl-6-[3-(methyloxy)phenyl]-N-(5,6J,8-tetrahydi naphthalen-1-yl)pyrimidin-4-amine;
6-(IH-indol-6-yl)-2-methyl-N—{(IS)—I-[3-(methyloxy)phenyl]ethyl}pyrimidin-4-amine;
2-niethyl-6-[3-(methy oxy)phenyl]-N-naphthalen-1-ylpyrimidin-4-amine;
2-methyl-6-[3-(methyloxy)phenyl]-N-[(IS)-1,2,3,4-letrahydi naphthalen-I-yl]pyrimidin-4-amine; 2-methyl-N-[(l)-I,2₁3,4-letra ydronaphthalen-1-yl]-6-{3-[(trifluoromethyl)oxy]phenyl}pyrimidin-4-amine;
N-{3-[I-({2-methyl-6-[3-(methyloxy)phenyl]pyrimidin-4-y1}amino)ethyl]phenyl} glycine;
N-({3-[I-({2-met yl-6-[3-(metliyloxy)phenyl]pyrimidin-4-yl}amino)ethyl]phenyl}sulfonyl)glycine;
methyl-{3-[({2-melhyl-6-[3-(metliyloxy)phenyl]pyrimidin-4-yl}amino)methyl]phenyl}glycinate;
N methyl-2-{[3-(I-{[2-melhy-6-(I-metliyl-1H-indol-6-yl)pyrimidin-4-yl]amino}ethyl)phenyl]oxy}acetamide;
2-methyl-6-[13-(methyloxy)phenyl]-N-{1-[3-(me iyloxy)phenyl]propyl}pyrimidin-4-amine;
6-(1,3-benzothiazol-6-yl)-2-methyl-N-(6-nitro-3,4-dihydro-2H-chromen-4-yl)pyrimidin-4-amine;
N-(4-{[6-(I,3-benzothiazol-6-yl)-2-methylpyrimidin-4-yl]amino}-3,4-dihydiO-2H-chramen-6-yl)acetamide;
2-methyl-6-(3-methyl-1-benzoi uran-5-yl)-N-[(1R)-1,2,3,4-teirahydronaphthalen-1-yljpynmidin-4-amiue;
6-(I-benzoriiran-5-yl)-2-methyl-N-[(IR)-1,2,3,4-tetrahydi naphthalen-I-yl]pyriinidin-4-amine:
6-(I-benzothien-5-yl)-2-melhyl-N-[(IR)-1,2,3,4-tetrahydiOnaphlhalen-I-yl]pyrimidin-4-amine;
6-(1,3-beirzothiazol-6-yl)-2-mcthyl-N-[(IR)-1,2,3,4-telrahydronaphthalen-I-yl]pyrimidin-4-amine;
2-methyl-6-(4-methyl-3J4-dihydro-2H-14-benzoxazin-6-yl)-N-[(IR)-1,2,3,4-tetrahydronaphthalen-1-yl]pyrimidin-4-amine;
2-methy-6-(3-methyl-I-benzoiuran-5-yl)-N—{(IS)—I-[3-(melhyloxy)phenyl]ethyl}pyrimidin-4-amine;
2-methyl-6-(I-methyl-2,3-dihydi-1H-indol-6-yl)-N-[(Ill)-1,2,3,4-tetrahydronaphthalen-1-y1]pyrimidin-4-amine;
6-(1,3-benzoxazol-6-yl)-2-methyl-N-[(I R)-I0.2,3,4-tetrahydronaphthalen-I-yjpyrimidin-4-amine;
6-(I-benzoiuran-5-yl)-2-methyl-N—{(IS)—I-[3-(methyloxy)phenyl]ethyl}pyrimidin-4-amine; 6-(2,3-diliydro-1,4-benzodio.in-6-yl)-2-methyl-N-[(I)-1,2,3,4-tetrahydronaphthalen-1-yl]pyrimidin-4-amine;
2-methyl-6-(1-methyl-1H-benzimidazol-6-yl)-N-[(IR)-1,2,3,4-tetrahydronaphthalen-1-yl]pyrimidin-4-amine;
6-(I-beirzothien-5-yl)-2-methyl-N—{(IS)—I-[3-(methyloxy)phenyljethyl}pyrimidin-4-amine:
6-(3-amino-4-methylphenyl)-2-methyl-N-[(IR)-1,2,3,4-tetrahydronaphthalen-I-yl]pyrimidin-4-amine;
2-methyl-N—{(IS)—I-[3-(methyloxy)phenyl]ethyl}-6-(4-methylphenyl)pyrimidin-4-amine:
6-(4-chloi phenyl)-2-methyl-N-{(1S)-1-[3-(melhy loxy)phenyl]ethyl} pyrimidin-4-amine;
6-(1,3-benzothiazol-6-yr)-2-metliyl-N-(2-methylbutyl)pyriiTiidin-4-amine;
2-methyl-6-(1-methyl-1H-indazol-6-yl)-N-[(I R)-1,2,3,4-tetrahydronaphthalen-1-yl]pyrimidin-4-amine;
6-(3-chloi phenyl)-2-methyl-N—{(IS)—I-[3-(melhyloxy)phenyl]ethyl}pyrimidin-4-amine;
6-(1,3-benzothiazol-6-yl)-2-methyl-N-[(IS)-1,2,3,4-tetrahydronaphthalen-I-yl]pyrimidin-4-amine;
6-(3,4-dihydro-2H-1,4-benzoxazin-6-yl)-2-methy I-N-[(1R)-1,2,3,4-tetrahydiOnaphthalen-I-yl]pynmidin-4-amine;
2-methyl-6-(I-methyl-1H-indol-4-yl)-N-[(rR)-1,2,3,4-tetrahydronaphthalen-I-yl]pyrimidin-4-amine;
6-(2-nuorophenyl)-2-n thyl-N—{(IS)-1-[13-(methyloxy)phenyl]ethyl}pyrimidin-4-amine;
6-(IH-indol-4-yl)-2-methyl-N-[(] R)-1,2,3,4-tetrahydronaphthalen-yjpyrimidin-4-amine;
6-(2,3-dihydro-I H-indol-6-yl)-2-methy!-N-[(I R)-1,2,3,4-tetrahydronaphthalen-I-yl]pyrimidin-4-amine:
6-(2,2-difluoro-I:3-benzodioxol-5-yl)-2-methyl-N-[(I R)-I,2,3,4-tetrahydronaphthalen-1-yl]pyrimiciin-4-amine;
6-(4-fliiorophenyl)-2-methyl-N—{(IS)—I-[3-(methyloxy)phenyl]ethyl}pyrimidin-4-amine; 2Miiethyl-N—{(IS)-1-[3-(ii thyloxy)phenyljethyl}-6-(2-methylphenyl)pyrimidin-4-amine;
6-(IH-indazol-5-yl)-2-methyl-N-[(I R)-1,2,3,4-tetrahydronaphthalen-1-yljpyrimidin-4-amine:
6-(1-ethyl-1H-indazol-6-y I)-2-methyl-N-[(1R)-1,2,3,4-tetrahydronaphthalen-1-yl]pyrimidin-4-amine;
6-(13-benzoxazol-6-yl)-2 ethyl-N—{(IS)—I-[3-(methyloxy)phenyl]ethyl}pyrimidin-4-amine;
4-{[6-(L3-benzothiazol-6-yl)-2-methylpyi″imidin-4-yl]amino}-4-[3-(methyloxy)phenyl]butanenitrile;
6-(I H-indol-7-yl)-2-methyl-N-[(1R)-I\hat{ }amine;
6-(I H-indazol-6-yl)-2-methyl-N-[(I R)-I,2,4-tetrahydronaphthalen-1-yl]pyrimidin-4-amine;
6-(IH-benzimidazol-6-yl)-2-methyl-N-[(IR)-1,2,3,4-tetrahydronaphthalen-I-yl]pyrimidin-4-amine;
6-(3-benzolhiazol-6-yl)-2-methyl-N—{(IR)-I-[3-(methyloxy)phenyl]ethyl}pynmidin-4-amine;
6-(I H-indol-4-yl)-2-methyl-N-{(I S)-1-[3-(methyloxy)phenyl]ethyl} pyrimidin-4-amine;
6-(IH-indol-7-yl)-2-methyl-N—{(IS)—I-[3-(methyloxy)phenyl]ethyl}pyrimidin-4-amine;
6-(2-chloropheiiyl)-2-methyl-{(IS)—I-[3-(methyloxy)phenyl]ethyl}pyrirnidin-4-amine;
2-melhyl-6-(I-methyl-1H-benzimidazo!-6-yl)-N—{(I S)-1-[3-(methyloxy)phenyljethyl}pyrimidin-4-amine;
N-[3-(dimethylamino)propyl]-3-({2-methyl-6-[3-(methyloxy)pheny]pynmidin-4-yl}amino)-3-[3-(methyloxy)phenyl]propanamide;
3-({2-methyl-6-[3-(methyloxy)phenyl]pyrimidin-4-yl}amino)-3-[3-(methy loxy)pheny 1] propan-1-o I;
6-(4-acetyl-34-dihydro-2H-1I4-benzoxazin-6-y!)-2-methyl-N-[(IR)-I, 2,3,4-tetrahydronaphlhalen-1-y I]pyrimidin-4-amine;
ethyl 3-({2-methyl-6-[3-(methyloxy)phenyl]pyrimidin-4-yl}amino)-3-[3-(methyloxy)phenyl]propanoate; 6-[3-(dimethylamino)phenyl]-2-methyl-N—{(IS)—I-[3-(methyloxy)phenyl]ethyl}pyiimidin-4-amine;
6-(1 I-1-indazol-5-yl)-2-methyl-N-{(1S)-1-[3-(melhyloxy)phenyl]ethyl}pyrimidin-4-amine;
ethyl N-{3-({2-methyl-6-[3-(methyloxy)pheny pyrimidin-4-yl}arnino)-3-[3-(methyloxy)phenyl]piOpanoyl}-beta-alaninate;
6-[4-(dimethylamino)phcnyl]-2-methyl-N-{(1S)-1-[3-(metliyloxy)phenyl]ethyl}pyrimidin-4-amine;
6-(1,3-benzothiazol-6-yl)-2-methyl-N—{(IS)—I-[3-(methyloxy)phenyl]ethyl}pyrimidin-4-amine;
6-[4-chloro-3-(methylainino)phenyl]-2-methyl-N-[(]R)-I,2,3,4-tetrahydronaphthalen-1-yl]pyrimidin-4-amine;
6-(4-chloi-3-methylphenyl)-2-mthyl-N-[(I)-I,2,3,4-tetrahydronaphthalen-I-yl lpyrimidin-4-amine;
2-fluoro-4-{2-rnet″ yl-6-[(IR)-I,2,3,4-tetrahydronaphthalen-1-ylamino]pyrimidin-4-yl}benzamide;
[(2-chloro-5-{2-methyl-6-[(IR)-I,2,3,4-tetrahydronaphthalen-1-ylamino]pyrimidin-4-yl}phenyl)amino]acetonitrile;
2-methyl-6-(4-methylpheny])-N(IR)-I,2a3,4-ietrahydronaphthalen-1-yl]pyrimidin-4-amine;
6-[4-chloi-3-(dimethylamino)plienyl]-2-metliyl-N-[(IR)-I, 2,3,4-tetrahydronaphthalen-1-yl]pyrimidin-4-amine;
6-(4-chlorophenyl)-2-methyl-N-[(I R)-I.2,3,4-tetrahydronaphthalen-I-ylJpyrimidin-4-amine;
2-methyl-6-[4-methyl-3-(methylamino)phenyl]-N-[(IR)-I, 2,3,4-tetrahydronaplithalen-1-yl]pyrimidin-4-amine;
6-(3,4-dichlorophenyl)-2-mefhyl-N-[(IR)-1,2,3,4-tetrahydronaphthalen-I-yl]pyrimidin-4-amine;
[(2-methyl-5-{2-methyl-6-[(I)-1,2,3,4-tetrahyclronaphthalen-1-ylamino]pyrimidin-4-yl}phenyl)amino]acetonitrile;
6-(4-chloro-3-fluoi plienyl)-2-methyl-N-[(I R)-1.2.3, 4-tetrahydronaphthalen-1-yljpyrimidin-4-amine;
2-chloro-5-{2-methyl-6-[(IR)-1,2,3,4-tetrahydronaphthalen-1-ylamino]pyrimidin-4-yl}benzonitrile; 2-methyl-N-[(IR)-1,2,3,4-tetrahydronapluhalcn-1-yl]-6-[4-(trifluoromethyl)phenyl]pyrimidin-4-amine;
6-(3-fluorophenyl)-2-methyl-N-[(]R)-1,2,3,4-tetiahydronaphthalen-yl]pyrimidin-4-amine;
6-[3-(dimethylamino)plienyl]-2-methyl-N-I′(1R)-1,2,3,4-tetrahydronaphthalen-1-yl]pyrimidin-4-amine;
2-methyl-6-(2-nielhylphenyl)-N-[(IR)-1,2,3,4-tetrahydronaphthalen-1-yl]pyriniidin-4-amine;
3-{2-methyl-6-[(1R)-1,2,3,4-tetrahydronaphthalen-1-ylamino]pyrimidin-4-yl}benzonitrile;
6-(3-chlorophenyl)-2-methyl-N-[(IR)-1,2J.4-tetrahydronaphthalen-yllpyrimidin-4-amine;
2-methyl-6-[4-(methylamino)phenyl]-N-[(] R)-1,2,3,4-tetrahydronaphthalen-I-yl]pyrimidin-4-amine;
6-(4-nuoi phenyl)-2-methyl-N-[(IR)-1,2,3,4-tetrahydronaphthalen-1-yl]pyrimidin-4-amine;
methyl 2-chloro-5-{2-methyl-6-[(IR)-K2,3,4-IetrahydiOnaphthalen-I-ylamino]pyrimidin-4-yl}benzoate:
6-(4-aminophenyl)-2-methyl-N-[(IR)-1,2,3,4-telrahydronaphthalen-yl]pyrimidin-4-amine;
6-(2-fluorophenyl)-2-methyl-N-[(I R)-1,2,3,4-tetrahydronaphthalen-1-yl]pyrimidin-4-amine;
6-(2-chlorophenyl)-2-methyl-N-[(IR)-I,2,3,4-tetrahydronaphthalen-1-yl]pyrimidin-4-amine;
4-{2-methyl-6-[(IR)-1,213,4-tetrahydronaphthalen-1-ylamino]pyrimidin-4-yl}benzonitrile;
6-(3-aminophenyl)-2-methyl-N-[(IR)-1.2,3,4-tetrahydronaphthalen-1-yl]pyrimidin-4-amine;
N-2-(2-chloro-5-{2-methyl-6-[(IR)-1,2,3,4-tetrahydronaphthalen-I-ylamino]pyrimidin-4-yl}phenyl)-N-methylglycinamide;
6-[4-(dimethylamino)phenyl]-2-methyl-N-[(IR)-1,2,3,4-tetrahydronaphthalen-I-yl]pyrimidin-4-amine:
2-chloro-N-methyl-5-{2-methyl-6-[(I R)-1.2,3,4-tetrahydronaphthalen-1-ylamino]pyrimidin-4-yl}benzamide; 2-melhyl-N-[(I R)-I,2,3,4-ielrahydronaphlhalen-I-yl]-6-{4-[(trifluoromethyl)oxy]plienyl}pyrimidin-4-amine;
3-{2-methyl-6-[(I R)-I,2,3,4-letrahydronaphthalen-I-ylamino]pyrimidin-4-yl}benzamide;
6-(3-amino-4-chlorophenyl)-2-methyl-[(1R)-1,2,3,4-tetrahydronaphthalen-1-yl]pyrimidin-4-amine;
4-{2-methyl-6-[(IR)-1,2,3,4-tetrahydronaphthalen-1-ylamino]pyrimidin-4-yl}benzamide;
2-methyl-N—{(IS)—I-[3-(metliyloxy)phenyl]ethyl}-N′-[(IR)-1,2;34-tetrahydronap t alen-1-yl]pyrimidine-4,6-diamine;
6-(1,3-benzothiazol-6-yl)-N-[(4R)-3,4-dihydro-2H-chromen-4-yl]-2-methylpyrimidin-4-amine;
6-(1,3-bcnzothiazol-6-yl)- -[(4S)-3,4-dihydro-2H
4-amine;
6-(1,3-benzothiazol-6-yl)-N-(3,4-dihydro-2H-chiOmen-4-yl)-2-methylpyrimidin-4-amine;
6-(I,3-benzothiazol-6-yl)-N-(3,4-dihydro-2H-1-bcnzothiopyran-4-yl)-2-melhy I pyrimidin-4-amine:
6-(1,3-benzothiazol-6-yl)-N-(3,4-dihydro-I H-2-benzothiopyran-4-yl)-2-methylpyrimidin-4-amine;
N-(5-{[6-(1,3-benzothiazol-6-yl)-2-methy]pynmidin-4-yl]amino}-5,6,7,8-tetrahydronaphthalen-2-yl)acetamide;
6-furan-3-yl-2-methyl-N-[(IR)-1,2,3,4-tetraliydronaphthalen-yl]pyrimidin-4-amine;
2-methyl-N—{(IS)—I-[3-(methyloxy)p enyl]ethyl}-6-phenylpyrimidin-4-amine;
6-(3-ethylphenyl)-2-methyl-N—{(IS)—I-[3-(niethyloxy)phenyl]et′hyl}pyrimidin-4-amine;
N˜I˜-[6-(],3-benzothiazol-6-yl)-2-methylpynmidin-4-yl]-1,2,3,4-tetrahydronaphthalene-1,6-diamine;
2-methyl-6-(3-methylphenyl)-N-[(I R)-1,2,3,4-tetrahydronaphlhalen-1-y]]pyrimidin-4-amine;
2-methyl-N—{(I S)—I-[3-(methyloxy)phenyl]ethyl}-6-(3-methylphenyl)pyrimidin-4-amine;
6-(1,3-benzothiazol-6-yl)-2-methyl-N-[6-Cmet′hyloxy)-I.2ₛ3,4-tetrahydronaphthalen-I-yl Jpyrimidin-4-amine; ′6-(1,3-benzothiazol-6-yl)-2-methyl-N-[5-(methylo.\y)-I₅2,3,4-tetrahydronaphthalen-1-yl]pyrimidin-4-amine;
6-(1,3-benzothiazol-6-yl)-2-methyl-N-(6-methyl-3ₛ4-dihydro-2H-chromen-4-yl)pyrimidin-4-amine;
6-(3-elhylphenyl)-2-methyl-N-[(1R)-I_{J}2,3,4\hat{ }etrahydronaplnhalen-1-yl]pyrimidin-4-amine;
2-metliyl-6-(2-methylpyridin-4-yl)-N-[(I R)-1,2,3,4-tetrahydronaphthalen-1-yl]pyrimidin-4-amine;
6-(1,3-benzotliiazol-6-yl)-2-methyl-N-[7-(nielhyloxy)-1,2,3,4-tetrahydronaphthalen-I-yl]pyrimidin-4-amine;
2-methyl-6-pyridin-4-yl-N-[(IR)-1,2:3,4 etrahydiOnaphthalen-1-yl]pyrimidin-4-amine;
2-methyl-N-{(]S)—I-[3-(methyloxy)phenyl]ethyl}-6-(2-methylpyridin-4-yl)pyrinn 4-amine;
2-methyl-6-(I H-pyrazol-4-yl)-N-[(I R)-1,2,3,4-tetrahydronaphthalen-1-yl]pynmidin-4-amine;
2-methyl-6-pyridin-yl-N-[(IR)-1,2,3,4 \hat{ }amine;
2-methyl-N-[(] R)-1,2,3,4-tetrahydronaphthalen-1-yl]-4,5′-bipyrimidin-6-amine;
2 iielhyl-N—{(IS)—I-[3-(methy!oxy)p enyl]ethyl}-6-pyridin-3-ylpyrimidin-4-aiTii
2-methy I-N-{(I S)-1-[3-(methyloxy)phenyl]ethyl}-6-pyridin-4-ylpyrimidin-4-amine:
6-(2,3\hat{ }ihydro-I-benzoi iran-5-yl)-2Mnethyl-N-[(IR)-1,2,3,4-tetraliydronaphthalen-I-yl]pyrimidin-4-amine;
2-methyl-6-[(E)-2-phenylethenyl]-N-[(IR)-1,2,3:4-tetrahydiOnaphthalen-I-yl]pyrimidin-4-amine;
6-(1,3-benzothiazol-6-yl)-N-(6-iluoiO-3,4-dihydro-2H-chroinen-4-yl)-2-methylpyi′imidin-4-amine;
(4-{2-methyl-6-[(I R)-1.2,3,4-tetrahydiOiiaphlhalen-I-ylamino]pyrimidin-4-yl}phenyl)methanol;
2-methyl-6-phenyl-N-[(IR)-1,2,34-tetrahydiOnaphthalen-yl]pynmidin-4-amine; 4-{2-methyl-6-[(IR)-I;2,3,4 etrahydiOnaplnlialen-1-ylamino]pyrimidin-4-yl}phen 6-(1,3-benzothiazol-6-yl)-2-melhyl-N-(2-mel yl-1,2,3,4-telrahydronaphlhalen-I-yl)pyrimidin-4-amine; 6-(1-benzofuran-2-yl)-2-meihyl-N-[(1R)-1,2,3,4-Ietrahydronaphthalen-1-yl]pyrimidin-4-amine;
6-[3:4-bis(methyloxy)phenyl]-2-niethyl-N-[(IR)-1;2,3,4-tetrahydronaphthalen-I-yl]pyrimidin-4-amine;
2-methyl-6-naphthalen-2-yl-N-[(IR)-I.2,3,4-tetrahydronaphthalen-1-yl]pyrimidin-4-amine;
3-{2-methyl-6-[(I R)-1.2,3,4-tetrahydiOnaplithalen-ylamino]pyrimidin-4-yl}phenol; N-[I-(3-bromophenyl)elhyl]-2-met yl-6-[3-(metliyloxy)phenyl]pynmidin-4-arnine; 3-{2-methyl-6-[(I R)-1.2,3,4-tetrahydronaphthalen-1-ylamino]pyrimidin-4-yl}benzaldehyde;
6-[2-nuoro-5-(metliyloxy)plienyl]-2\hat{ }ethyl-N(I)-1:2,3,4-tetraliydronaphthalen-I-yl]pyrimidin-4-amine;
6-(1,3-benzothiazol-6-yl)-N-(5,7-dimethyl-1,2,3,4-tetrahydronaphthalen-1-yl)-2-methylpyrimidin-4-amine;
1-(3-{2-methyl-6-[(I R)-1.2,3,4-tetrahydronap Ihalen-1-ylaminojpyrimidin-4-yl}phenyl)ethanone;
(3-{2-methyl-6-[(I R)-1,23,4 etraliydronaphthalei 1-ylamino]pyrimidin-4-yl}phenyl)methanol;
6-[3-(ethyloxy)phenyl]-2-methyl-N-[(IR)-1,2,3,4-tetrahydronaphthalen-I-yl]pyrimidin-4-amine;
2-methy]-6-(4-methyl-3,4-dihydro-2H-1,4-benzoxazi n-7-yl)-N-[(1R)-1,2,3,4-tetrahydronaphthalen-1-yl]pyrimidin-4-amine;
N-[1-(3-{[(1,3-dimethyl-I H-pyrazol-5-yl)methyl]oxy}phenyl)ethyl]-2-methyl-6-(I-methyl-1H-indol-6-yl)pyrimidin-4-amine;
6-(1,3-benzothiazol-6-yl)-N-[(1S)-1-(3-{[(1,3-dimethyl-1H-pyrazol-5-yl)methyl]oxy}phenyl)ethyl]-2-methylpyiimidin-4-amine;
2-nuoro-5-(I-{[2-methyl-6-(3-methyl-I-benzoi ira]i-5-yl)pyrimidin-4-yl]amino}ethyl)phenol;
N-{-[4-nuoro-3-(I-methyl-1H-pyrazol-4-yl)phenyl]ethyl}-2-methyl-6-(3-methyl-I-benzofuran-5-yl)pyrimidin-4-amine;
6-[2-chloiO-3-(methyloxy)phenyl]-N-[-(3-{[(1,3-dimelhyl-1H-pyrazol-5-yl)methyl]oxy}phenyl)ethyl]-2-melhylpyrimidin-4-amine;
2-methyl-6-[4-methyl-3-(methyloxy)phenyl]-N-{(1S)-1-|3-(methyloxy)phenyl]ethyl}pyrimidin-4-amine;
3-[(]S)-1-{[6-(1,3-benzothiazol-6-yl)-2-methylpyrimidii 4-yl]amino}ethyl]phenol;
N-[I-(3-{[(1,3-dimet1 iyl-1H-pyrazol-5-yl)methyl]oxy}phenyl)ethyl]-2-methyl-6-[4-metliyl-3-(methyloxy)phenyljpyrimidin-4-amine;
6-(1,3-benzothiazol-6-yl)-2-methyl-N-(!-{3-[(IH-pyrazol-3-ylmelhyl)oxy] phenyl} ethyl)pyrimidin-4-amine;
3-(I-{[6-(1,3-benzothiazol-6-yl)-2-methylpyiimidin-4-yl]amino}ediyl)-N-(cyanomethyl)benzenesulfonairiide;
6-(1,3-benzothiazol-6-yl)-N-(I-{3-[(isoxazol-3-ylmethyl)oxy]phenyl}ethyl)-2-methylpyrimidin-4-amine;
3-(I-{[6-(1,3-benzothiazol-6-yr)-2-methylpyrimidin-4-yl]amino}ethyl)-N-but-2-yn ylbenzenesulfonamide;
2-methyl-6-[4-methyl-3-(methyloxy)phenyl]-N-[!-(3-{[(I-methyl-IH-pyrazol-3-yl)methyl]oxy}phenyl)ethyl]pyrimidin-4-amine;
6-(13-benzothiazol-6-yl)-N-[I-(3-{[(1,3-dimethyl-1H-pyrazol-5-yl)methyl]oxy}-4-fluoi phenyl)ethyl]-2-methylpyrimidin-4-amine;
2-({3-[I-({2-methyl-6-[4-methyl-3-(methyloxy)phenyrjpynmidin-4-yl}amino)ethyl]phenyl}oxy)acetamide;
3-(I-{[6-(1,3-benzodiiazol-6-yl)-2-methylpyrimidin-4-yl]amino}ethyl)-N-prop-2-yn-1-ylbenzenesulfonamide;
6-(1,3-benzothiazol-6-yr)-N-{I-[4-fluoro-3-(I-methyl-1H-pyrazol-4-yl)phenyl]ethyl}-2-methylpyrimidin-4-amine;
{[3-(I-{[6-(1,3-benzolhiazol-6-yl)-2-methylpyrimidin-4-yl]amino}ethyl)phenyl]oxy}acetonitrile;
N-(1-{-[(2-azepan-1-yl-2-oxoetliyl)oxy]phenyl}elhyl)-6-(1,3-benzothiazol-6-yl)-2-methylpyrimidin-4-amme;
2-{[3-(1-{[6-(1,3-benzothiazol-6-yl)-2-methylpyrimidin-4-vl]amino}elhyl)phenyl]oxy}-N-(I-methylethyl)acetamide;
6-(2,3-dihydro-1-benzo(uran-5-yl)-2-methyl-N-{(1S)-1-[3-(methyloxy)phenyl]ethyl}pyrimidin-4-amine;
6-(1,3-benzothiazol-6-y I)-2-methyl-N-[1-(3-{[2-(2-methy laziridin-1-y 0-2-oxoethyl]oxy}phenyl)ethyl]pyrimidin-4-amine;
2-{[3-(1-{[6-(1,3-benzothiazol-6-yl)-2-methylpyrimidin-4-yl]amino}ethyl)phenyl]oxy}-N,N-dimethylacetamide; 3-(1-{[6-(1,3-benzothiazol-6-yl)-2-methylpyrimidin-4-yl]amino}ethyl)benzenesulfonamide;
6-(1,3-benzothiazol-6-yl)-2-methyl-N-[I-(3-{[(5-methylisoxazol-3-yl)methyl]oxy}phenyl)etliyl]pyrimidin-4-amine;
{[3-(I-{[2-methyl-6-(I-methyl-1H-indol-6-yl)pyrimidin-4-yl]amino}ethyl)phenyl]oxy}acelonitrile;
6-(I.3-benzothiazol-6-yl)-2-methyl-N-{1-[13-(prop-2-yn-1-ylpxy)phenyl]etliyl}pynmidin-4-amine;
N-{1-[2-fluo!O-3-(methyloxy)phenyl]ethyl}-2-methyl-6-(3-methyl-1-benzo uran-5-yl)pyrimidin-4-amine;
2-chloro-5-(I-{[2-methyl-6-(3-methyl-1-benzofuran-5-yl)pyrimidin-4-yljamino}ethyl)plienol;
3-[1-({2-methyl-6-[4-methyl-3-(methyloxy)phenyl]pyrimidin-4-y1}amino)ethyl]phenol;
6-(1,3-benzothiazol-6-yl)-2-methyl-N-(I-{3-[(2-oxo-2-piperidin-ylethyl)oxy]phenyl}ethyl)pyrimidin-4-amine;
N-(1-{3-[(2-azetidin-1-yl-2-oxoethyl)oxy]phenyl} ethyl)-6-(I,3-benzothiazol-6-yl)-2-methylpyrimidin-4-amine:
2-{[3-(1-{16-(1,3-benzotliiazol-6-yi)-2-met!iylpyriiTiidin-4-yl]amino}ethyl)phenyl]oxy}-N-(2-hydroxypropyl)acetamide;
3-(I-{[6-(1,3-benzothiazol-6-yl)-2-melhylpyrimidin-4-yl]amino}ediyl)-N-(2-hydi xyethyl)benzenesul fonamide;
6-(1,3-benzothiazol-6-yl)-N-[I-(5-{[(1,3-dimethyl-II-1-pyrazol-5-yl)methyl]oxy}-2-nuorophenyl)ethyl]-2-methylpyiimidin-4-ainine;
3-(I-{[6-(1,3-benzothiazol-6-yl)-2-methylpyrimidin-4-yl]amino}ethyl)-N-[2-(methyloxy)ethyl]benzenesulfonamide;
3-[(I R)-1-{[6-(1,3-benzoUiiazol-6-yl)-2-me iylpyrimidin-4-yl]amino}ethyl]phenol;
6-(1,3-benzolliiazol-6-yl)-2-methy]-N-(I-{3-[(2-morpholin-4-yl-2-oxoethyl)oxy]phenyl}ethyl)pyrimidin-4-amine;
3-(I-{[6-(1,3-benzolhiazol-6-yl)-2-methylpyrimidin-4-yl]amino}ethyl)-4-fluorophenol;
3-[I-({6-[2-fluoiO-3-(methyloxy)plienyl]-2-methylpyrimidin-4-y1}amino)ethyl]phenol; N,N-diethyl-2-({3-[1-({6-[2-iliioro-3-(methyloxy)phenyl]-2-methylpyrimidin yl}amino)ethyl]phenyl}oxy)acetamide;
6-(1,3-benzothiazol-6-yl)- -[(1R).1-(3-{[(1,3-dimethyl-1H-pyrazol-5-yl)methyl]oxy}phenyl)ethyl]-2-methylpyrimidin-4-amine;
6-(1,3-benzothiazol-6-yl)-2-methyl-N-(I-{3-[(2-oxo-2-pyrrolidin-ylethyl)oxy]phenyl}ethy])pyriniidin-4-amine;
2-methyl-6-(I-inetliyl-1H-indol-6-yl)-N-[I-(3-{[2-oxo-2-(4-pyridin-2-ylpiper yl)ethyl]oxy}phenyl)etliyl]pyrimidin-4-amine;
methyl 1-({[3-(1-{[2-melhyl-6-(1-methyl-1H-indol-6-yl)pyrimidin-4-yl]amino}ethyl)phenyl]oxy}acetyl)piperidine-4-carboxylate;
2-{[3-(1-{[6-(la 3-benzolhiazol-6-yl)-2-methylpyrimidin-4-yrjamino}ethyl)phenyl]oxy}-N-methylacetamide;
2-{[3-(1-{[6-(1,3-benzothiazol-6-yl)-2-methylpyrimidin-4-yl]amino}ethyl)phenyl]oxy}-ethylacetamide;
3-(1-{[6-(1,3-benzothiazol-6-yl)-2-methylpyrimidin-4-yjamino}ethyl)phenol {[3-(I-{[2-methyl-6-(1-methyl-1H-indol-6-yl)pyrimidin-4-yl]amino}ethyl)phenyl]oxy}acetic acid;
4-{[3-(I-([6-(1,3-benzothiazol-6-yl)-2-methylpyi′imidin-4-yl]amino}ethyl)phenyl]oxy}butanoic acid;
ethyl 4-{[3-(1-{[6-(1,3-benzothiazol-6-yl)-2-methylpyrimidin-4-yl]amino}ethyl)phenyl]oxy}butanoate:
1,1-dimethylethyl (3S)-3-({[6-(I.3-benzothiazol-6-yl)-2-methylpyrimidin-4-y]amino}methyl)piperidine-I-carboxylate:
2-{[3-(1-{[6-(1,3-benzothiazol-6-yl)-2-methylpyrimidin-4-yl]amino}ethyl)phenyl]oxy}-N,N-diethylacetamide:
6-(4-chlo! phenyl)-2-melhyl-N-{I-[3-(I-methyl-1H-pyrazol-4-yl)phenyl]ethyl}pyrimidin-4-amine;
6-(3-fluorophenyl)-2-methyl-N-{1l-[3-(I-methyl-IH-pyrazol-4-yl)phenyl]ethyl}pyrimidin-4-amine;
2-methyl-6-[3-(methyloxy)phenyl]-N-{1-[3-(1-methyl-1H-pyrazol-4-yl)phenyl]ethyl}pyrimidin-4-amine;
6-(I H-indol-5-yl)-2-methyl-N-{I-[3-(I-methyl-111-pyrazol-4-yl)phenyl]ethyl}pyrimidin-4-amine; 2-methyl-6-(3-methylp enyl)-N-{1-[3-(1-methyl-1H-pyrazol-4-yl)phenyl]ethyl}pyrimidin-4-amine;
6 I,3-benzothiazol-6-yl)-N-[(2-chloro-6-nuorophenyl)methyl]-2-methylpyrimidin-4-amine;
2-methyl-N-{13-(I-methyl-1H-pyrazol-4-yl)phenyl]ethyl}-6-phenylpyrimidin-4-amine;
2-methyl-6-[4-(methyloxy)phenyl]-N-{I-[3-(I-methyl-1H-pyrazol-4-yl)phenyl]ethyl}pyrimidin-4-amine;
6-(2,4-dichlorophenyl)-2-methyl-N-{1-[3-(I-methyl-IH-pyrazol-4-yl)phenyl]ethyl}pyrimidin-4-amine;
2-methyl-6-(2-methylphenyl)-N-{1-[3-(1-methyl-1H-pyrazol-4-yl)phenyl]ethyl}pyrimidin-4-amine;
6-(3-chloiO-4-iluorophenyl)-2-methyl-N-{1-[3-(I-methyl-1H-pyrazol-4-yl)phenyl]ethyl} pyrimidin-4-amine;
N-{2-[3-({[6-(1,3-benzothiazol-6-yl)-2-methylpyrimidin-4-yl]amino}methyl)piperidin-1-yl]-2-oxoelhyl}-N-methylbenzamide;
2-methyl-N-{I-[3-(I-methyl-IH-pyrazol-4-yl)phenyl]ethyl}-6-naphthalen-2-ylpyrimidin-4-amine;
6-(1,3-benzothiazol-6-yl)-2-methyl-N-[(2R)-2-phenylpropyl]pyrimidin-4-amine;
6-(1,3-benzothiazol-6-yl)-2-melhyl-N-(2-pyridin-3-ylethyl)pyrimidin-4-amine;
6-(13-benzothiazol-6-yl)-N-(2-ethylhexyl)-2-methylpyrimidin-4-amine;
6-(1,3-benzothiazol-6-yl)-2-methyl-N-(I-pyridin-3-ylethyl)pyrimidin-4-amine;
6-(1,3-benzothiazol-6-yl)-N-(2,3-dihydi-IH-inden-2-yl)-2-methylpyrimidin-4-amine;
6-(1,3-benzothiazol-6-yl)-N-(1,4-dimethylpenty)-2-methylpyrimidin-4-amine;
6-(1,3-benzothiazol-6-yl)-N-[2-(I H-imidazol-4-yl)ethyl]-2-methylpyrimidin-4-amine; 6-(1,3-benzothiazol-6-yl)-2-methyl-[(2S)-2-phenylpropyl]pyrimidin-4-amine;
5-{f6-(1-benzothiazol-6-yl)-2-methylpyrimidiiv4-yl]amino}-2,2-dimethylpentan-I-ol;
6-(1,3-benzothiazol-6-yl)-2-melhyl-N-{[3-(IH-pyrrol-1-yl)phenyl]methyl}pyrimidin-4-amine;
1,1-dimethylethyl 3-({[6-(1,3-benzolhiazol-6-yl)-2-methylpyrimidin-4-yl]amino}methyl)piperidine-1-carboxylate;
6-(I J-benzothiazol-6-yl)-2-methyl-N-(2-pyridin-4-ylethyl)pyrimidin-4-amine; 6-(1,3-benzotlnazol-6-yl)-2-methyl-N-[(3-phenylisoxazol-5-yl)methyl]pynmidin-4-amine;
N′-[6-(1,3-benzotlnazol-6-yl)-2-methylpyrimidin-4-yl]-N-methyl-N-phenylpiOpane-1,3-diamine;
2-methyl-6-(3-methyl-I
I-benzofuran-5-yl)-N—{
I-[3-(I H-tetrazol-1-yl)phenyl]etliyl}pyrimidin-4-amine;
2-methyl-6-(3-methyi-1-benzofuran-5-yl)-N-{1-[3-(4H-1,2,4-triazol-4-yl)plienyl]elhyl}pyrimidin-4-amine;
2-melhyl-6-(3-methyl-1-benzofuran-5-yl)-N-[I-(3 iitrophenyl)ethyl]pyrimidin-4-amine;
2-methyl-6-[4-methyl-3-(met yloxy)phenyl]-N-[(IR)-I,2,3,4-tetrahydronaphthalen-I-yljpynmidin-4-amine;
6-(1,3-benzothiazol-6-yl)-2-methyl-N-{1-[3-(1H-1,2,3-triazol-1-yl)phenyl]ethyl}pyrimidin-4-amine;
N-[3-(1-{12-methyl-6-(3-methyl-1-benzofxiran-5-yl)pyrimidin-4-yl]amino}ethyl)phenyl]prop-2-enamide;
2-mcthyl-6-(1-methyl-1H-indol-6-yl)-N-{1-[3-(I-mclh 1-1H-pyrazol-4-y!)phenyl]ethyl}pyrimidin-4-amine;
N-[I-(3-aminophenyl)ethyl]-2-methyl-6-(3-methyl-1-benzofuran-5-yl)pyrimidin-4-amine;
6-(1,3-benzothiazol-6-yl)-2-methyl-{1-[3-(5-methyl-I H-tetrazol-1-yl)phenyl]ethyl}pyrimidin-4-amine;
2-methyl-6-[4-methyl-3-(prop-2-yn-1-yloxy)phenyl]-N-[(IR)-I 2,3,4-tetrahydronaphthalen-1-y I]pyrimidin-4-amine;
6-(1,3-benzothiazol-6-yl)-2-methyl-N-{1-[3-(1l-1-letrazol-I-yl)phenyl]ethyl}pyrimidin-4-amine;
6-(I,3-benzothiazol-6-yl)-2-methyl-N-{1-[3-(4H-1,2,4-triazol-4-yl)phenyl]ethyl}pyrimidin-4-amine;
3-(I-{[6-(1,3-benzolhiazol-6-yl)-2-methylpyrimidin-4-yl]amino}ethyl)benzonitrile; 2-{[3-(I-{[6-(1,3-benzothiazol-6-yl)-2-methylpyrimidin-4-yl]amino}ethyl)phenyl]oxy}-,N-diethylpropanamide:
2-{[3-(I-{[6-(1,3-benzothiazol-6-yl)-2-methylpyrimidin-4-yllamino}ethyl)phenyl]oxy}-2-methylpropanamide; 6-(1,3-benzothiazol-6-yl)-2-methyl-N-{]-[3-(5-methyl-L2,4-oxadiazol-3-yl)phenyl]ethyl}pyrimidin-4-amine;
2-{[3-(I-{[6-(1,3-benzothiazol-6-yl)-2-methylpyiimidin-4-yl]amino}ethyl)phenyl]oxy}propanamide;
3-(I-{[6-(1,3-benzothiazol-6-yl)-2-methylpyrimidin-4-yl]amino}ethyl)benzenecarboximidamide;
N-[3-(I-{[6-(] 3-benzot iazol-6-yl)-2-met ylpyiimidin-4-yl]amino}ethyl)phenyl]-1H-pyrazole-5-carboxamide;
N-[3-(I-{[6-(1,3-benzothiazol-6-yl)-2-methylpynmidin-4-yl]amino}ethyl)phenyl]-1-methyl-1H-pyrazole-3-carboxamide;
2-methyl-5-{2 i thyl-6-[(IR)-1,2J,4-teiraliydronaphthalen-ylamino]pyrinn*din-4-yl}phenol;
N-[3-(I-{[6-(1,3-benzot iazol-6-yl)-2-melhylpyrimidin-4-yl]amino}ethyl)phenyl]dicarbonimidic diamide;
N-[3-(I-{[6-(1,3-benzothiazol-6-yl)-2-methylpyrimidin-4-yl]amino}elhyl)phenyl]IOrmainide;
1-[3-(I-{[6-(1,3-benzolhiazol-6-y!)-2-niethylpyrimidin-4-yl]amino}ethyl)phenyl]urea;
N-[3-(I-{[6-(13-benzothiazol-6-yl)-2-methylpyrimidin-4-yl]amino}ethyl)pheny]]-1-methy-1H-imidazole-4-SLilfonamide:
6-(1,3-benzothiazol-6-yl)-2-methyl-N-{1-[3-(2H-tetrazol-5-yl)phenyl]ethyl}pyrimidin-4-amine;
1,1-dimethylethyl (2-{[3-(I-{[6-(13-benzothiazol-6-yl)-2-methylpyrimidin-4-yl]amino}elhyl)phenyl]amino}-2-oxoelhyl)methylcarbamate;
3-(I-{[6-(1,3-benzodiiazol-6-yl)-2-methylpyrimidin-4-yl]amino}ethyl)-N′-hydroxybenzenecarboximidamide;
N-[6-(1,3-benzothiazol-6-yl)-2-methylpy amine;
N-[6-(1,3-benzothiazol-6-yl)-2-met ylpynmidin-4-yl]-I-methyl-1,2,3,4-tetrahydroq inolin-4-amine;
2-methy]-6-(3-methyl-]-benzof ran-5-yl)-N-[(IS)—I-(3-pyrimidin-5-ylphenyl)ethyl]pyrimidin-4-amine;
5-{3-[(IS)-1-{[2-methyl-6-(3-methyl-1-benzofuran-5-yl)pyrimidin-4-yl]amino}ethyl]phenyl}pyrimidin-2-amine; N—{(IS)—I-[3-(6-aminopyriclin-3-yl)phenyl]ethyl}-2-methyl-6-(3-methyl-I-benzofuran-5-yl)pyrimidin-4-amine;
ethyl (4-{3-[(IS)-1-{[2-methyl-6-(3Hiiethyl-1-benzoluran-5-yl)pyrimidin-4-yl]amino}ethyl]phenyl}-I H-pyrazol-I-yl)acetate;
2-methyl-1-{[3-(I-{[2-methyl-6-(3-methyl-1-benzo uran-5-yl)pynmidin-4-yl]amino}ethyl)phenyl]oxy}propan-2-ol;
1-{[3-(I-{[2-methyl-6-(3-methyl-1-benzofuran-5-yl)pyrimidin-4-yl]amino}ethyl)phenyl]oxy}propan-2-one;
6-(3-ethyl-I-benzoturan-5-yl)-2-melhyl-N-[(IR)-1.2,3,4-tetrahydrohaphthalen-I-yl]pyrimidin-4-amine;
6-(I-benzothien-5-yl)-N-(4R)-3,4-dihydro-2H-chromen-4-yl]-2-methylpyrimidin-4-amine;
(4-{3-[(IS)-1-{[2-methyl-6-(3-methyl-1-benzofuran-5-yl)pynmidin-4-yl]amino}ethyl]phenyl}-1H-pyrazol-1-yl)acetic acid;
6-(1,3-benzothiazol-6-yl)-2-methyl-N-[(IS)—I-(3-pyrimidin-5-ylphenyl)ethyl]pyrimidin-4-amiiie:
2-methyl-6-(I-methyl-1H-indol-2-yl)- -[(IR)-1,2,3,4-telrahydronaphthalen-I-yl]pyrimidin-4-amine;
2-methyl-6-(3-methyl-1-benzofuran-5-yl)-N-[I-(3-{[(5-methyl-1,2,4-oxadiazol-3-yl)methyl]oxy}phenyl)ethyl]pyrimidin-4-amine;
5-[3-(1-{[6-(1,3-benzothiazol-6-yl)-2-methylpyrimidin-4-yl]amino}ethyl)phenyl]pyrimidin-2-amine;
2-methyl-6-(1-methyl-1H-pyrrolo[3,2-b]pyridin-6-yl)-N-[(I R)-1,2, 3,4-tetrahydronaphthalen-1-yl]pyrimidin-4-amine;
ethyl (4-{3-[(1S)-1-{[6-(1,3-benzothiazol-6-yl)-2-methylpyrimidin-4-yl]amino}ethyl]phenyl}-I H-pyrazol-I-yl)acetate;
6-(7-fluoro-1-benzofuran-5-yl)-2-methyl-{(IS)—I-[3-(methyloxy)phenyl]ethyl} pyrimidin-4-amine;
6-(4-ethyl-3,4-dihydiO-2H-1,4-benzoxazin-6-yl)-2-melhyl-N-[(IR)-I, 2,3,4-tetrahydroniiphthalen-1-yl]pyrimidin-4-amine;
2-methyl-6-(3-methyl-1-benzofuran-5-yl)-N-[l-(3-methylphenyl)elhyi]pyrimidin-4-amine;
6-(1,3-benzothiazol-6-yl)-N-(3-{[(I-ethylpiperidin-3-yl)methyjoxy}phenyl)ethyl]-2-methylpyrimidin-4-amine; N-{I-[3-(6-aminopyridin-3-yl)p enyl]ethyl}-6-(1,3-benzothiazol-6-yl)-2-methylpyrimidin-4-amine;
6-(13-benzothiazol-6-yl)-2-melhyl-N-[I-(3-{[(1-methylpiperidin-3-yl)metliyl]oxy}phenyl)ethyl]pyrimidin-4-aminc;
N-[1-(3-{[(1,3-dimethyi-1H-pyrazol-5-yl)methyl]oxy}phenyl)ethyl]-2-methyl-6-(I-methyl-1H-indol-2-yl)pyrimidin-4-amine;
2-methyl-6-(3-methyl-1-benzofuran-5-yl)-N-[(I S)-1-(4-methylphenyl)ethyl]pyrimidin-4-amine;
N-[1-(3-{[(1,3-dimethyl-1H-pyrazol-5-yl)methyl]oxy}phenyl)ethyl]-6-(3-ethyl-1-benzoluran-5-yl)-2-methylpyrimidin-4-amine;
6-(1,3-benzothiazol-6-yl)-2-methyl-N-(1-{3-[(piperidin-3-y methyl)oxy]phenyl}ethyl)pyrimidin-4-amiiie;
1-{[3-(1-{[6-(1,3-benzothiazol-6-yl)-2-methylpyrimidin-4-yl]amino}ethyl)phenyl]oxy}propan-2-one:
6-(1,3-benzothiazo]-6-yl)-2-methyl-N-{1-[3-(2-methyl-I,3-thiazol-4-yl)phcnyl]ethyl}pyrimidin-4-amine;
6-(1,3-benzothiazol-6-yl)-N-[I-(5-{[(K3-dimethyl-II-1-pyrazo]-5-yl)methyl]oxy}pyridtn-3-yl)ethyl]-2-methylpyrimidin-4-amine;
6-(3-ethyl-1-benzofuran-5-yl)-2-methyl-Ts-{(IS)—I-[3-(methyloxy)phenyl]ethyl}pyrimidin-4-amine;
2-methyl-6-(3-methyl-1-benzofuran-5-y I)-{(1R)-1-[3-(1-methyl-1H-pyrazol-4-yl)phenyl]ethyl}pyrimidin-4-amine;
2-{[3-({[6-(1,3-benzothiazol-6-yl)-2-melhylpyrimidin-4-yl]amino}methyl)-4-′fluorophenyl]oxy}-N,N-diethylacetatnide;
2-methyl-6-(3-methyl-I-benzoliiran-5-yl)-N-{[3-(methyloxy)phenyl]methyl}pyrimidin-4-amine;
1-{[3-(I-{[6-(13-benzothiazol-6-yl)-2-methylpyrimidin-4-yl]amino}ethyl)phenyl]oxy}propan-2-ol;
1-{[3-(1-{[6-(1,3-benzothiazol-6-yl)-2-methylpyrimidin-4-yl]amino}ethyl)phenyl]oxy}-2-methylpiOpan-2-ol;
N-[I-(2-nuorophenyl)ethyl]-2-methyl-6-(3-methyl-I-benzoixiran-5-yl)pyrimidin-4-amine;
3-(m-{[6-(3-ethyl-I-benzoturaiv5-yl)-2-methylpynmidin-4-yl]amino}ethyl)pheno\hat{ } 2-methyl-6-(I-methyl-1H-indol-2-yl)-N—{(IS)—I-[3-(methyloxy)phenyl]ethyl}pyiimidin-4-amine;
6-(1,3-benzolhiazol-6-yl)- -[I-(2-cliloropyndin-4-yl)ethyj-2-methylpyrimidin-4-amine;
e-ilJ-benzothiazol-e-y \hat{ }-metliyl-N-f1-S-ICiS-methyl-l\hat{ }\hat{ }-oxadiazol-S-yl)methyl]oxy}phenyl)ethyl]pyrimidin-4-amine;
N-[I-(3-{[(I-acetylpiperidin-3-yl)methyl]oxy}phenyl)ethyl]-6-(1,3-benzothiazol-6-yl)-2-methylpyrimidin-4-amine;
2-{[3-(I-{[2-methyl-6-(I-melhyl-1H-indol-2-yl)pynmidin-4-yl]amino}ethyl)phenyl]oxy}acetamide;
methyl {f(2-chloro-5-{2-methyl-6-[(IR)-L2,3,4-tetrahydronaphthalen-I-ylamino]pyrimidin-4-yl}phenyl)methyl]oxy} acetate;
6-(1,3-bcnzothiazol-6-yl)-2-metliyl-N-(I-{3-[(piperidin-4-ylmethyl)oxy]phenyl}ethyl)pyrimidin-4-amine;
2-methyl-6-(4-methyl-3,4-dihydi-2H-1,4-benzoxazin-6-yl)-N-{1-[3-(I-methyl-1H-pyrazol-4-yl)phenyl]ethyl}pyrimidin-4-amine;
N-[I-(3-{[2-(4-acetylpiperazin-I-yl)ethyl]oxy}phenyl)ethyl]-6-(1,3-benzothiazol-6-yl)-2-methylpyrimidin-4-amine;
6-(1,3-benzothiazol-6-yl)-N-{[2-bromo-5-(melhyloxy)phenyl]methyl}-2-methylpyrimidin-4-amine;
2-{[3-({[6-(1,3-benzothiazol-6-yl)-2-methylpyrimidin-4-yl]amino}methyl)-4-fluoiOphenyl]oxy}-N,N-dimethylacetamide;
6-(I-beiizothien-2-yl)-2-methyl-N-[(I)-1,23,4-tetrahydronaphthalen-I-yl]pyrimidin-4-amine;
{4-[3-(I-{[6-(1,3-benzothiazol-6-yl)-2-methylpyrimidin-4-ylJamino}ethyl)phenyl]-1H-pyrazol-1-yl}acetic acid;
6-(3-ethyl-1-benzofuran-5-yl)-2-methyl-N-{1-[3-(1-methyl-1H-pyrazol-4-yl)phenyl]ethyl}pyrimidin-4-amine;
2-{[3-(I-{6-(])3-benzothiazol-6-yl)-2-methylpyrimidin-4-yrjamiiio}ethyl)plienyl]oxy}-N-[2-(metliyloxy)ethyl]acetamide;
3-({[6-(1,3-benzothiazol-6-yl)-2-methylpyrimidin-4-yl]amino}methyl)-4-fluorophenol;
6-(IH-indol-2-yl)-2-methyl-N-[(I R)-1.2.3;4-tetrahydronaphthalen-1-y]pyrimidin-4-amine; 2-{[3-(1-{[6-(1,3-benzothiazol-6-yl)-2-methylpyrimidin-4-yl]amino}ethyl)phenyl]oxy}-1-cyclopropylethanone;
2-{[3-(I-{[6-(1,3-benzothiazol-6-yl)-2-methylpyrimidin-4-yl]amino}ethyl)phenyl]oxy}-N-phenylacetamide;
6-(I-benzofuran-2-yl)-2-nielliyl-N—{(IS)—I-[3-(mcthyloxy)phenyl]ethyl}pyrimidin-4-amine;
6-(I)₃-benzothiazol-6-yl)-N-({2-Iluoro-5-[(2-morpholin-4-yl-2-oxoethyl)oxy]phenyl}methyl)-2-methylpyrimidin-4-amine;
1,1-dimethylethyl 3-({[3-(1-{[6-(I,3-benzothiazol-6-yl)-2-niethylpyrimidin-4-yl]amino}ethyl)phenyl]oxy}melhyl)piperidine-1-carboxylate;
1,1-dimethylethyl 4-({[3-(I-{[6-(1,3-benzothiazol-6-yl)-2-methylpyrimidin-4-yl]amino}ethyl)phenyl]oxy}meVhyl)piperidinc-1-carboxylate;
6-(1,3-benzothiazol-6-yl)-2-methyl-N-(I-{3-[6-(methyloxy)pyridin-3-yl]phenyl}ethyl)pyrimidin-4-amine;
N N-diethyl-2-{[3-(I-{[2-methyl-6-(4-methyl-3:4-dihydro-2H-l14-benzoxazin-6-yl)pyrimidin-4-yl]amino}ethyl)phenyl]oxy}acetamide;
2-methyl-{(1S)-1-[3-(melhyloxy)phenyl]ethyl}-6-(1-methyl-1H-pyrrolo[3,2-b]pyridin-6-yl)pyrimidin-4-amine;
6-(1,3-benzothiazol-6-yl)-N-[(5-bromo-2-fluorophenyl)methyl]-2-methylpyrimidin-4-amine;
2-methyl-6-(3-methyl-1-benzofuran-5-yl)-N-[I-(2-melhylphenyl)ethyl]pyrimidin-4-amine;
6-(1,3-benzothiazol-6-yl)-N-{[2-nuoi-5-(methyloxy)phenyl]methyl}-2-metliylpyrimidin-4-amine:
6-(1,3-benzothiazol-6-yl)-N-[(5-{[(L3-dimethyl-1H-pyrazol-5-yl)methyl]oxy}-2-nuoiOphenyl)melhyl]-2-methylpyrimidin-4-amine:
methyl {[3-({[6-(1,3-benzolhiazol-6-yl)-2-methylpyrimidin-4-yl]amino}methyl)-4-nuorophenyl]oxy}acetate;
6-(1,3-benzothiazol-6-yl)-2-mehyl-N—{(IR)-I-[3-(1-methyl-1H-pyrazol-4-yl)phenyl]ethyl}pyrimidin-4-amine;
-(IS)-3-{[3-(1-{[6-(I;3-benzothiazol-6-yl)-2-methylpyrimidin-4-yl]amino} ethyl)pheny I]oxy}-1-phenylpropan-1-ol;
(2-chloro-5-{2-methyl-6′(IR)-L2:3;4-tetrahydronaphthalen-1-ylamino]pyrimidin-4-y 1} pheny I)methanol;
6-(3-chloro-4-methylphenyl)-2-methyl-N-[(I)-1,2,3,4-tetrahydronaphthalen-1-yl]pyrimidin-4-amine;
N-(2-hydroxyet yl)-2-(5-{2-methyl-6-[(IR)-l:2,3,4-tetrahydronaphthalen-I-ylamino]pyrimidin-4-yl}-1-benzofuran-3-yl)acetamide;
2-(6-{2-methyl-6-[(I R)-1,2,3;4-tetrahyd] naphtlialen-1-ylamino]pyrimidin-4-yl}-2.3-dihydro-4H-1,4-benzoxazin-4-yl)ethanol;
2-(6-{2-methyl-6-[(I R)-1,2,3,4-tetrahydronaphthalen-1-ylamino]pyrimidin-4-yl}-2,3-dihydro-1H-indol-1-yl)ethanol;
ethyl (5-{2-methyl-6-[(1R)-1,2,3,4-tetrahydronaphthalen-1-ylamino]pyrimidin-4-yl}-1-benzofuran-3-yl)acetate;
methyl (6-{2-methyl-6-[(IR)-1,2,3,4-tetrahydronapht′halen-1-ylamino]pyrimidin-4-y I}-2,3-dihydro-1H-indol-1-yl)acetate;
N-[(4R)-3,4-dihydro-2i-I-chromen-4-yl]-2-methyl-6-(3-methyl-1-benzofuran-5-yl)pyrimidin-4-amine;
NaN-diethyl-2-{[3-(]-{[2-methyl-6-(3-methyl-1-benzofuran-5-yl)pynmidin-4-yl]amino}ethyl)phenyl]oxy}acetamide;
6-[4-chloro-3-(prop-2-yn-1-ylamino)phenyl]-2-methyl-N-[(IR)-1,2,3,4-tctrahydronaphthalen-1-ylJpyrimidin-4-amiiie;
N-[1-(3-{[21H-imidazol-1-yl)ethyl]oxy}phenyl)ethyl]-2-methyl-6-(3-methyl-1-benzofuran-5-yl)pyrimidin-4-amine;
N—{(IS)-1-[3-(I-ethyl-IH-pyrazol-4-yl)phenyl]ethyl}-2-methyl-6-(3-methyl-1-benzofuran-5-yl)pyrimidin-4-amine;
6-(4-chloro-3-methylphenyl)-N-[(4R)-3,4-dihydro-2H-chromen-4-yl]-2-methylpyrimidin-4-amine;
2-methyl-6-(3-methyl-1-benzofuran-5-yl)-N-(1-{3-[(piperidin-3-ylmethyl)oxy]phenyl}ethyl)pyrimidin-4-amine;
2-methyl-6-(3-methyl-1-benzofuran-5-y I)-N-{(1S)-1-[3-(1-methyl-1H-pyrazol-5-yl)phenyl]ethyl}pyrimidin-4-amine;
2-{[3-(1-{[2-methyl-6-(3-methyl-1-benzofiiran-5-yl)pyrimidin-4-yl amino}ethyl)phenyl]oxy}ethanol;
2-methyl-6-(3-methyl-1-benzofuran-5-yl)-N-[(IS)-1-{3-[(2-morpholin-4-yl-2-oxoethyl)oxy]phenyl}ethylJpyrimidin-4-amine;
N-[I-(3-{[3-(dimethylamino)propyl]oxy}phenyl)ethyl]-2-methyl-6-(3-methyl-I-benzo uran-5-yl)pyrimidin-4-amine; 2-methyl-6-(3-methyl-I-benzof′iiran-5-yl′)-N-[I-(3-{[(1-meth 1-1H-pyrazol-3-yl)methyl]oxy}phenyl)ethyl]pyrimidin-4-amine;
3-(1-{[2-methyl-6-(3-methyl-I-benzoiu³% 5-yl)pyrimidin-4-yl]amino}ethyl)phenol; N-[(1S)-1-(3-bromophenyl)ethyl]-2-methyl-6-(3-methy 1-1-benzofuran-5-yl)pyrimidin-4-amine;
2-methyl-6-(3-methyl-1-benzofiiran-5-y 1)-N-{(1 S 1-[3-(1H-pyrazol-4-yl)phenyllethyl}pyrimidin-4-amine;
3-[(1S)-1-{[2-methyl-6-(3-methyl-I-benzofuran-5-yl)pyrimidin-4-yl]ammo}ethyl]phenol;
2-methyl-6-(3-methyl-1-benzofuran-5-yl)-N-{(1S)-1-[3-(1-methyl-IH-pyrrol-2-yl)phenyl]ethyl}pyrimidin-4-amine;
N-[(4R)-3:4-dihydro-2H hromen-4-yl]-2-methyl-6 4-methyl-3,4-dihydiO-2H-1,4-benzoxazin-6-yl)pyrimidin-4-amine:
2-metliyl-6-(3-methyl-1-benzol iran-5-yl)-N(IS)—I-phenylethyl]pyrimidin-4-amine;
6-(1,3-benzothiazol-6-yl)-2-methyl-N-[I-(3-pyridin-3-ylphenyl)ethyl]pyrimidin-4-amine:
6-(I,3-benzothiazol-6-yl)-2-methyl-N-{1-[3-(1-methyl-1H-pyrazo!-4-yl)phenyl]ethyl}pyrimidin-4-amine;
N-[(IS)—I-(4-fliioi phenyl)ethyl]-2-methyl-6-(3-methyl-I-benzofia-an-5-yl)pyrimidm\hat{ } 4-amine;
6-(1-benzofuran-5-yl)-N-[1-(3-{[(1,3-dimethyl-1H-pyrazol-5-yl)methyl]oxy}phenyl)elhyl]-2-methylpyrimidin-4-amine;
6-(I;3-benzothiazol-6-yl)-2-methyl-N—{(I S)-I-[3-(I-methyl-1H-pyrazol-5-yl)phenyl]ethyl}pyrimidin-4-amine;
N-[I-(3-fluorophenyl)ethyl]-2-methyl-6-(3-methyl-1-benzofuran-5-yl)pyrimidin-4-amine;
6-(1,3-benzothiazol-6-yl)-N—{(IS)—I-[3-(I-ethyl-1H-pyrazol-4-yl)phenyl]ethyl}-2-methylpyrimidin-4-amine;
2-methyl-6-[4-methyl-3-(pi p-2-yn-I-ylamino)phenyrj-N-[(I)-1,2,3,4-tetrahydronaphthalen-1-yl]pyrimidin-4-amine;
2-methyl-6-(3-methyl-1-benzol′uran-5-yl)-N-{(1S)-1-[3-(1H-pyrazol-5-yl)phenyl]ethyl}pyrimidin-4-amine;
2-methyl-6-(3-methyl-I-benzofiiran-5-yl)-N-[(IR)-1-{3-[(2-morpholin-4-yl-2-oxoethyl)oxy]phenyl}ethyl]pyrimidin-4-amine;[(5-{6-[(4R)-3,4-dihydro-2H-chromen-4-ylamino]-2-methylpyrimidin-4-yl}-2-melhylphenyl)amino]acetonitrile;
3-[(IR)-1-{[2-metliy]-6-(3-methyl-1-benzofuran-5-yl)pyri!′nidin-4-yl]amino}ethyl]phenol;
6-(1,3-benzothiazol-6-yl)-2-methyl-N—{(IS)—I-[3-(IH-pyrazol-4-yl)phenyl]ethyl}pyrimidin-4-amine;
6-(1,3-benzothiazol-6-yl)-2-methyl-N-f 1-(3-{[(rnelhylsulibnyl)methyl]oxy}phenyl)ethyl]pyrimidin-4-amine;
[(2 1 uoro-5-{2-methyl-6-[(rR)-1,2,4-Ietiahydronaphthalen-ylamino]pyrirnidin-4-yl}phenyl)aminojacetonilrile;
2-methyl-6-(3-methyl-I-benzofuian-5-yl)-N—{(IR)-1-[3-(methyloxy)phenyl]ethyl}pyrimidin-4-amine;
2-(methyloxy)-4-{2-methy I-6-[(1R)-1,2,3,4-tetrahydronaphthalen-1-ylamino]pyrimidin-4-yl}benzamide;
6-(1,3-benzothiazol-6-yl)-2-methyl-N-(I-{3-[(2-morpholin-4-ylethyl)oxy]phenyl}ethyl)pyrimidin-4-amine;
6-(1,3-benzothiazol-6-yl)-2-methyl-N-{(1S)-1-[3-(1-methyl-1H-pyrral-2-yl)phenyl]ethyl}pyrimidin-4-amine;
6-(1,3-benzothiazol-6-yl)-2-methyl-N-1-(3-{[2-(2-methyl-1H-imidazol-1-yl)ethyl]oxy}phenyl)ethyl pyrimidin-4-amine;
6-(I-benzoiiiran-5-yl)-2-methyl-N-[I-(3-{[(I-methyl-1H-pyrazol-3-yl)methylloxy}phenyl)elhyl]pyrimidin-4-amine;
N-[(]S)-1-biphenyl-3-ylethyl]-2-methyl-6-(3-methyl-1-benzo uran-5-yl)pyrimidin-4-amine;
4-{[3-(I-{[6-(1,3-beiizothiazol-6-yl)-2-methylpyrimidin-4-yl]amino}ethyl)phenyl]oxy} butan-1-ol:
6-(1,3-benzothiazol-6-yl)-2-methyl-N-1-(3-morpholin-4-ylphenyl)ethyl]pyrimidin-4-amine;
3-{[3-(I-{[6-(1,3-benzothiazol-6-yl)-2-methylpyrimidin-4-yl]amino}ethyl)phenyl]oxy} propane-1,2-diol;
6-(I J-benzothiazol-6-y -N—I(IS)-1-biphenyl-3-ylclhyl]-2-niethylpyrimidin-4-amine; 2-{[3-(1-{[6-(1,3-benzothiazol-6-yl)-2-methylpyrimidin-4-yl]amino}ethyl)phenyl]oxy}ethanol; 1-{[3-(I-{[6-(1,3-benzothiazol-6-yl)-2-methylpyrimidin-4-yl]amino}ethyl)phenyl]oxy}-3-fluoi propan-2-ol:
6-(1,3-Bεηζo11ïαζ(ol-6\hat{ }1)-N-[1-(3-ΓθπioρHoηγ1)ε\hat{ }1]-2-ηï6ïιγ1ρ>′ππï\acute{α}ïη-4-aηiíηε;
3-{[3-(I-{[6-(1,3-bcnzothiazol-6-yl)-2-metliylpyrimidin-4-yl]amino}ethyl)phenyl]oxy}piOpan-1-ol;
4-{[3-(I-{[6-(1,3-benzothiazol-6-yl)-2-methylpyrimidin-4-yl]amino}ethyl)phenyl]oxy}-,N-dimethylbutanamide;
6-(1,3-benzothiazol-6-yl)-N-1-(3-{[3-(diethylamino)propyl]oxy}phenyl)ethyl]-2-methylpyrimidin-4-amine;
6-(1,3-benzothiazo!-6-yl)-2-methyl-N-[1-(3-{[2-(1H-pyrrol-1-yl)ethyl]oxy}phenyl)ethyl]pyrimidin-4-amine;
6-(]:3-benzothiazol-6-yl)-2-metby!-N-(1-{3-[(3-morpholin-4-ylpropyl)oxy]phenyl}ethyl)pyrimidin-4-amine;
2-{[3-(1-{[6-(1,3-benzothiazol-6-y)-2-methylpyrimidin-4-yl]amino}ethyl)phenyl]oxy}-N-(2-hydroxyethyl)acetamide;
2-{[3-(1-{[6-(,3-benzothiazol-6-yl)-2-methylpyrimidin-4-y]amino}ethyl)phenyl]oxy}-cyclopropylacetamide;
6-(I,3-benzothiazol-6-yl)-2-methyl-N-{1-[3-(1H-pyrrol-2-yl)phenyl]ethyl} pyrimidin-4-amine;
6-(1 benzothiazol-6-yl)-2-methyl-N-[1-(3-{[2-(1H-pyrazol-1-yl)ethyl]oxy}pheny)ethyl]p)Timidin-4-amine;
N,N-diethyl-2-[(3-{1I-[(2-methyl-6-naphthalen-2-ylpyrimidin-4-yl)amino]ethyl}phenyl)oxy]acetamide;
6-(1,3-benzothiazol-6-yl)-2-melhyl-(I-{3-[(3-pyrrolidin-I-ylpropyl)oxy]phenyl}ethyl)pyrimidin-4-amine;
6-(1,3-benzothiazol-6-yl)-N-[1-(3-{[3-(dimethylamino)pipyl]oxy}phenyl)ethyl]-2-methylpyrimidin-4-amine;
6-(1a3-benzothiazol-6-yl)-2-methyl-N-{1I-[2-Cmethyloxy)pyridin-4-yl]ethyl}pyrimidin-4-amine;
1,1-dimethylethyl3-({[3-(I-{f2-methyl-6-(3-methyl-1-benzofuran-5-yl)pyrimidin-4-y]amino}ethyl)phenyjoxy}methyl)piperidine-1-carboxylate;
2-{[3-(I-{[6-(1,3-benzothiazol-6-yl)-2-methylpyrimidin-4-yl]amino}ethyl)phenyl]oxy}-N-cyclohexylacetamide; 6-(1,3-benzotlnzol-6-yl)-N-[I-(3′-nuoiObiphenyl-3-yl)ethyl]-2-met ylp)Timidin-4-amine;
2-methyl-6-quinolin-6-yl-N-[(IR)-1,2,3,4 etrahydronaphthalen-yl]pyrimidin-4-amine:
6-(1,3-benzothiazol-6-yl)-N-[(2-Iluoropheiiyl)methyl]-2-niethylpyrinn\hat{ }in-4-amine; 2-methyl-6-quinoxalin-6-yl-N-[(I)-1,2,3,4-tetrahydronaphthalen-1-yl]pyrimidin-4-amine;
methyl {[3-(I-{[6-(1,3-benzothiazol-6-yl)-2-methylpynmidin-4-yl]amino}ethyl)phenyl]oxy} acetate;
6-(1,3-benzothiazol-6-yl)-2-methyl-N-[I-(3-{[2-(methylsuHOnyl)ethyl]oxy}plienyl)ethyl]pyrimidin-4-amine;
6-(3-amino-4-methylphenyl)-2-methyl-N—{(IS)-]-[3-(methyloxy)phenyl]ethyl} pyrimidin-4-amine;
2-methyl-N—{(I S)—I-[3-(methyloxy)phenyl]ethyl}-6-quinolin-6-ylpyrimidin-4-arnine
{[3-(I-{[6-(1,3-benzolhiazol-6-yl)-2-methylpyrimidin-4-yl]amino}ethyl)pheny]]oxy}acetic acid;
6-(1,3-benzothiazol-6-y[)-2-metbyl-N-(piperidin-3-ylmethyl)pyrimidin-4-amine;
6-(3-amino-4-chlorophenyl)-N-(4R)-3,4-dihydro-2H-chromen-4-yl]-2-methylpyrimidiri-4-amine;
6-(1-benzothiazol-6-yl)-N-[(2-chlorophenyl)methyl]-2-methylpyrimidin-4-amine;
6-(1,3-benzotliiazol-6-yl)-N-[(2-chloro-6-fluoro-3-methylphenyl)methyl]-2-methylpyrimidin-4-amine;
6-(1,3-benzothiazol-6-yl)-N-{[2-chloro-6-niioro-3-(methyloxy)phenyl]methyl}-2-methylpyrimidin-4-amine;
2-mothyl-6-(3-methyl-1-benzofuran-5-yl)-N—{(IS)—I-[3-(I-methyl-1H-pyrazol-4-yl)plienyl]ethyl}pyrimidin-4-amine;
6-(1,3-benzothiazol-6-yl)-2-methyl-N-{(1S)-1-[3-(1-methyl-1H-pyrazol-4-yl)phenyl]ethyl}pyrimidin-4-amine;
N N-dimethyl-2-({3-[(IS)-1-{[2-methyl-6-(3-methyl-1-benzofuran-5-yl)pyrimidin-4-yl]amino}ethyl] phenyl}oxy)acelamide;
N-[I-(3-{[2-(dimethylamino)ethyl]oxy}phenyl)ethyl]-2-methyl-6-(3-methyl-I-benzofuran-5-yl)pyrimidin-4-amine;
N,N-dimethyl-2-({3-[(IR)-1-{[2Hiiethyl-6-(3-methyl-1-benzofuran-5-yl)pyrimidin-4-yl]amino}ethyl]phenyl}oxy)acetamide; 6-(1,3-benzotln′azol-6-yl)-N-[I-(3-{[2-(dimelhylamino)ethyl]oxy}phenyl)ethyl]-2-methylpyrimidin-4-amine;
6-(1,3-benzothiazol-6-yl)-2-methyl-N-{[1-(IH-pyrrol-2-ylcarbonyl)piperidin-3-yl]methyl}pyrimidin-4-amine;
6-(2,5-dimethylphenyl)-2-methyl-N-{1-|3-(1-methyl-1 I-I-pyrazol-4-yl)phenyl]ethy!}pyrimidin-4-amine;
6-(3,4-dichloiOpheny])-2-methyl-{I-[3-(I-methyl-1H-pyrazol-4-yl)phenyr]ethyl}pyrimidin-4-amine;
6-(4-ethylphenyl)-2-methyl-N-{I-[3-(I-methyl-1H-pyrazol-4-yl)phenyl]elhyl}pyrimidin-4-amine;
6-(4-iluoro-3-methylphenyl)-2-methyl-N-{1-[3-(I-methyl-IH-pyiazol-4-yl)phenyl]ethyl}pyrimidin-4-amine;
6-(1,3-benzothiazol-6-yl)-2-methyl-N-({1-[(1-methy Icyclopropyl)carbony I]piperidin-3-yl}methyl)pyrimidin-4-amine;
6-(13-benzothiazol-6-yl)-N-{[1-(cyclopentylcarbonyl)piperidin-3-yl]methyl}-2-methylpyrimidin-4-amine;
2-methyl-6-[4-(1-methylethyl)phenyl]-{1-[3-(I-methyl-1H-pyrazol-4-yl)phenyl]ethyl}pyrimidin-4-amine;
e-f1\hat{ }-benzothiazol-e-yrj-N-ffl-\hat{ }yclobutylcarbony piperidin-S-ylJmethyl}\hat{ }-riiethylpyrimidin-4-amine;
6-[2-fluoiO-4-(methyloxy)phenyl]-2-metliyl-N-{I-[3-(I-methyl-1H-pyrazol-4-yl)phenyl]ethyl}pyrimidin-4-amine;
6-(1,3-benzolhiazol-6-yl)-N-{[1-(cyclohexylcarbonyl)piperidin-3-yl]methyl}-2-m ethylpyri m i d i n-4-amine;
6-[3-(dimethylamino)phenyl]-2-methyl-N-{1-[3-(I-methyl-I H-pyrazol-4-yl)phenyl]ethyl}pyrimidin-4-amine;
6-(1,3-benzothiazol-6-yl)-2-methyl-N-(I-methylbutyl)pyrimidin-4-amine;
6-(1,3-benzothiazol-6-yl)-2-methyl-N-(3-methylbutyl)pyrimidin-4-amine;
6-(1,3-benzothiazol-6-yl)-N-(2-cyclohex-1-en-1-ylethyl)-2-methylpyrimidin-4-amine;
4-(2-{[6-(I;3-benzothiazol-6-yl)-2-melhylpyiimidin-4-yl]amino}ethyl)phenol;
6-(1,3-benzothiazol-6-yl)-2-methyl-N-pentylpyrimidin-4-amine;
6-(1,3-benzothiazol-6-yl)-2-methyl-N-(I-methylpiOpyl)pyrimidin-4-amine;
6-(I,3-benzothiazol-6-yl)-2-methyl-N-(I-methylhexyl)pyrimidin-4-amine; 6-(la3 ienzothiazol-6-yl)-2-met yl-N-[(IS)-1,2,2-trimethylpi pyl]pyrimidin-4-amine;
6-(13-benzothiazol-6-yl)-2 netliyl- -(2-pyndin-2-ylethyl)pyrirnidin-4-amine; 6-(I,3-benzothiazol-6-yl)-2-methyl-N-[l-methyl-2-(methyloxy)elhyl]pyrimidin-4-amine;
6-(L3-benzothiazol-6-yl)- -[(I S)—I-(3-biOmophenyl)ethyl]-2-melhylpyrimidin-4-amine;
6-(K₃-benzothiazol-6-yl)-2-methyl-N—{(IS)—I-[3-(IH-pyrazol-5-yl)phenyl]ethyl}pyrimidin-4-amine;
(2E)-3-{3-[(I S)—I-{[6-(I,3-benzotliiazol-6-y)-2-mediylpyrimidin-4-yl]amino}ethyl]phenyl}-N-ethylpiOp-2-enaniide;
2-{13-(1-{[6-(1,3-benzothiazol-6-yl)-2-methylpyrimidin-4-yl]amino}ethyl)phenyl]oxy}-N-[2-(dimethylamino)ethyl]acetamide;
2-{[3-(1-{[6-(1,3-benzothiazol-6-yl)-2-methylpyrimidin-4-yl]amino}ethyl)phenyl]oxy}-N-piOpylacetamide;
2-{[3-(I-{[6-(1,3-benzothiazol-6-yl)-2-methylpyrimidin-4-yl]amino}ethyl)phenyjoxy}-N-ethyl-N-methylacetamide:
6-(13-benzotliiazol-6-yl)-2-methyl-N-[I-(3-nitrophenyl)ethyl]pyrimidin-4-amine;
(2E)-3-{3-[(IS)-1-{[6-(L3-benzothiazol-6-yl)-2-methylpyrimidin-4-yl]amino}ethyl]phenyl}-N-(1, 1-dimethylethyl)prop-2-enamide;
N-[I-(3-aminophenyl)ethyl]-6-(1,3-benzothiazol-6-yl)-2-mediylpyrimidin-4-amine;
2-({[3-(I-{[6-(1,3-benzothiazol-6-yl)-2-methylpyrimidin-4-yl]amino}ethyl)phenyl]oxy} methyl)-1,3-oxazole-4-carboxylic acid;
methyl 2-({[3-(1-{[6-(1,3-benzothiazol-6-yl)-2-methylpyrimidin-4-yl]amino}ethyl)phenylloxy} methyl)-1,3-oxazole-4-carboxylate;
6-(1,3-benzothiazol-6-yl)-2-methyl-N-[(IS)-1-phenylethyl]pyrimidin-4-amine;
6-(I_{J}3-benzolhiazol-6-yl)-N-(2:2-dimethy-3,4-dihydiO-2H-chromen-4-yl)-2-methylpyrimidin-4-amine;
[(2-methyl-5-(2-methyl-6-1 (1R)-1,2,3,4-tetrahydronaphthalen-1-ylamino]pyrimidin-4-yl}phenyl)oxy]acetonitrile;
2-methyl-6-(2-phenylethyl)-N-[(I R)-1,2,3,4-tetrahydiOnaphthalen-1-yl]pyrimidin-4-amine;
2-melhyl-6-(3-methyl-1-benzofuran-5-yl)-N—{(IS)—I-[4-(methyloxy)phenyl]ethyl}pyrimidin-4-amine; 6-(]i3-benzothiazol-6-yl)-N-[I-(3-{[(1,3-dimethyl-1H-pyrazol-5-yl)methyl]oxy}phenyl)ethyl]-2-methylpyrimidin-4-amine;
N-[I-(3-fiiran-3-ylphenyl)ethy]]-2-methyl-6-(3-methyl-1-benzofuran-5-yl)pyrirnidin-4-amine;
6-(1,3-benzothiazol-6-yl)-2-methyl-N-(1-{3-[(phenylmetliyl)oxy]phenyl}ethyl)pyrimidin-4-amine:
2-methyl-6-(3-met yl-1-benzofuran-5-yl)-N—{I-[3-(3-diienyl)phenyl]ethyl}pyrimidin-4-amine;
6-(1,3-benzothiazol-6-yl)-2-metliyl-N-(1-{3-[(pyridin-3-ylmethyl)oxy]phenyl}ethyl)pyrimidin-4-amine;
6-(1,3-benzothiazol-6-yl)-2-metliyl-[I-(3-{[(3-methylisoxazol-5-yl)methyl]oxy}phenyl)ethyl]pyrimidin-4-aminc;
6-(1,3-benzothiazol-6-yl)-2-methyl-N-[I-(3-{[(2-methyl-1,3-thiazol-4-yl)met yl]oxy}phenyl)ethyl]pyrimidin-4-amine;
6-(I a3-benzothiazol-6-yl)-2-methyl-{]-[3-(propyloxy)phenyl]ethyl}pyrimidin-4-amine;
6-(1a3-benzothiazol-6-yl)-N-{1-f3-(3,5-dimethylisoxazol-4-yl)phenyl]ethyl}-2-methylpyrimidin-4-amine;
6-{2-methyi-6-[(I)-1,2,3,4-tetrahydronaphthalen-ylarnino]pyrirnidin-4-yl}-4H-chromen-4-one;
6-(13-benzothiazol-6-yl)-2-methyl-N-[I-(3-{[(4-methylp enyl)methyl]oxy}phenyl)ethyl]pynmidin-4-amine;
6-(13-benzothiazol-6-yl)-N-[I-(3-furan-3-ylphenyl)ethyl]-2-methylpyrimidin-4-amine;
6-(I:3-benzothiazol-6-yl)-2-methyl-N-f I-(3-{[(3-{[(4-methylphenyl)oxy]methyl}-1,2,4-oxadiazol-5-yl)methyl]oxy}phenyl)elhyrjpyrimidin-4-amine;
6-(13-benzothiazol-6-yl)-2-methyl-N-{1-[3-(3-thienyl)phenyl]ethyl}pyrimidiri-4-amine;
6-(2:1:3-benzoxadiazol-5-yl)-2-methyl-N-[(I R)-1,2,3,4-tetrahydronaphthalen-I-yl]pyrimidin-4-amine;
2-melhyl-6-{[3-(methyloxy)phenyl]oxy}-N-[(I RH\hat{ }\hat{ }\hat{ }-tetrahydronaphthalen-I-yl]pyrimidin-4-amine;
N-(5-{[6-(113-benzothiazo]-6-yl)-2-methylpyrimidin-4-yl]amino}-5,6,7,8-tetrahydronaphthalen-2-yl)propanamide; 2-methyl-6-(I-melhyl-1H-indol-5-yl)-N-[(IR)-1,213,4-tetrahydronaphthalen-I-yl]pyrimidin-4-amine;
6-( )-benzothiazol-6-yl)-N-f1-(3-{[2-(IH-imidazol-1-yl)ethyl]oxy}phenyl)ethyl]-2-niethylpyrimidin-4-amine;
2-{[3-(I-{[2-methyl-6-(3-methyl-1-benzofuran-5-yl)pyrimidin-4-yl]amino}ethyl)phenyljoxy}acetamide;
N-[I-(3-{[(1,3-dimethyl H-pyrazol-5-yl)rnethyl]oxy}phenyl)ethyl]-2-methyl-6-(3-methyl-1-benzofuran-5-yl)pynmidin-4-amine;
2-{[3-(I-{[6-(1,3-beiizolhiazol-6-yl)-2-methylpyrimidin-4-yl]amino}ethyl)phenyl]oxy}acetamide;
2-methyl-6-(3-mefhyl-I-benzofuran-5-yl)-N-(I-{3-[(2-morpholin-4-yl-2-oxoethyl)oxy]phenyl}ethyl)pyrimidin-4-aniine;
2-methyl-6-quinolin-3-yl-N-[(I R)-1,2,3,4-tetrahydronaphthalen-1-yl]pyrimidin-4-amine;
N,N-dimethyl-2-{[3-(I-{I\hat{ }-methyl-6-(3-methy]-1-benzofuran-5-yl)pyrimidin-4-yl]amino}ethyl)phenyl]oxy}acetamide;
6-(L3-benzothiazol-6-yl)-2-methyl-N-[I-(3-{[(I-methyl-1H-pyrazol-3-yl)methyl]oxy}phenyl)efhyl]pyrimidin-4-amine;
6-(1,3-benzothiazol-6-yl)-N-(I-{3-[(2-fluoroelhyl)oxy]phenyl}ethyl)-2-methylpyrimidin-4-amine;
6-(1,3-benzothiazol-6-yl)-2-methyl-N-(I-{3-[(2,2>2-trifluoroethyl)oxy]phenyl}ethyl)pyrimidin-4-amine;
N-[I-(3-bromo-4-floiOphenyl)elhyl]-2-methyl-6-(3-methyl-1-benzofuran-5-yl)pyrimidin-4-amine;
5-[2-fluoiO-5-(1-{[2-methyl-6-(3-methyl-1-benzofuran-5-yl)pyrimidin-4-yl]amino}ethyl)plienyl]pyrimidiii-2-amine;
N-{(1R)-I-[4-fluoro-3-(I-methyl-1H-pyrazol-4-yl)phenyl]ethyl}-2-methyl-6-(3-methyl-1-benzofuran-5-yl)pyrimidin-4-amine;
N—{(IS)—I-[4-iluoro-3-(]-methyl-1H-pyrazol-4-yl)phenyl]ethyl}-2-methyl-6-(3-methyl-1-benzofuran-5-yl)pyrimidin-4-amine;
5-[3-(1-{16-(1,3-benzolhiazol-6-yl)-2-methylpyrimidin-4-yl]amino}ethyl)phenyl]pyridine-3-carboxaniide;
6-(1,3-benzothiazol-6-yl)-2-metliyl-N-[(IS)-1-{3-[2-(4-methylpiperazin-I-yl)pyrimidin-5-yl]phenyl}ethyl]pyrimidin-4-amine; 6-(1,3-benzothiazol-6-yr)-2-methyl-N-(I-{3-[5-(trifluoiOmethyl)pyridin-3-yl]phenyl}ethyl)pynmidin-4-amine;
2-methy]-6-(3-methyl-1-benzofuran-5-yl)-N-[(IS)-1-{3-[2-(4-methylpiperazin-I-yl)pyrimidin-5-ylJphenyl}ethyllpyrimidin-4-amine;
3-[(5-{3-[(IS)-1-{[2-methyl-6-(3-melhyl-1-benzoiuran-5-yl)pyniTiidin-4-yl]amino}ethyl]phenyl} pyrimidin-2-yl)amino]propan-1-ol methyl N-(5-{3-[(I S)-1-{[2Hiielhyl-6-(3-methyl-1-benzo(uran-5-yl)pyrimidin-4-yl]amino}ethyl]phenyl}pyrimidin-2-yl)glycinate;
2-methyl-6-(3-methyl-1-benzofuran-5-yl)-N-[(IS)-1-{3-[6-(4-methylpiperazin-I-yl)pyndin-3-yl]phcnyl}etliyl]pyrimidin-4-amine
2,2-dimethyl-3-[(5-{3-[(1S)-1-{[2-melhyl-6-(3-methyl-1-benzol:′Liran-5-y])pyrimidin-4-yl]amino}ethyl]phenyl}pyriniidin-2-yl)aniino]piOpan-I-ol;
-(5-{3-[(IS)-1-{[2-methyl-6-(3-methyl-I-benzoiuran-5-yl)pyrimidin-4-yl]amino}ethyl]phenyl}pyrimidin-2-yl)glycine; ′
3-[(5-{3-[(IS)-1-{[2-melhyl-6-(3-met yl-1-benzofuran-5-yl)pyrimidin-4-yl]amino}ethyl]phenyl}pyrimidin-2-yl)amino]propane-1,2-diol;
N-[(1S)-1-(5′-fluoi-3,3′-bipyridin-5-yl)ethyl]-2-methyl-6-(3-methyl-1-benzofuran-5-yl)pyrimidin-4-amine;
N-[(l S)-1-(6,]uoro 3′-bipyndin-5-yl)ethyl]-2-methyl-6-(3-methyl-I-benzofuran-5-yl)pyrimidin-4-amine;
N-{(1S)-1-[5-(1-ethyl-1H-pyrazol-4-yl)pyridin-3-yl]ethyl}-2-methyl-6-(3-methyl-1-benzofuran-5-yl)pyrimidin-4-amine;
6-chloro-5′-[(IS)-1-{[2-methyl-6-(3-methyl-1-benzofuran-5-yl)pyrimidin-4-yl]amino}ethyl]-3,3′-bipyridin-5-amine;
5-{5-[(IS)-1-{[2-methyl-6-(3-methyl-1-benzofuian-5-yl)pyrimidin-4-yl]amino}ethyl]pyridin-3-yl}pyrimidin-2-amine;
N 1-(3-bromo-5-nuorophenyl)ethyl]-2-metliyl-6-(3-methyl-1-benzofuran-5-yl)pyrimidin-4-arnine;
5-[3-Iluoro-5-(I-{[2-melhyl-6-(3-methyl-1-benzofuran-5-yl)pyrimidin-4-yl]amino}ethyl)phenyl]pyrimidin-2-amine;
N-{I-[3-fluoro-5-(I-methyl-1H-pyrazol-4-yl)phenyl]ethyl}-2-niethyl-6-(3-methyl-I-benzofuran-5-yl)pyrimidin-4-amine;
I-(5-{5-[(IS)-1-{[2-methyl-6-(3-methyl-1-benzofuran-5-yl)pyrimidin-4->4]amino}ethyl]pyridin-yl}pyrimidin-2-yl)piperidin-4-ol; N-{I-[3-(5-amino-6-chloropyridin-3-yl)-5-nuorophenyl]ethyl}-2-methyl-6-(3-methyl-I-benzofuran-5-yl)pyrimidin-4-amine;
N-[I-(3-bromo-4-melliylphenyl)ethyl]-2-methyl-6-(3-methyl-I-benzoii.iran-5-yl)pyrimidin-4-amine;
5-[2-methyl-5-(I-{[2-methyl-6-(3-methyl-I-benzofuran-5-yl)pyiimidin-4-yl]amino}ethyl)phenyl]pyrimidin-2-amine;
2-methyl-6-(3-methyl-I-benzofuran-5-yl)-N-{1-[4-methyl-3-(I-methyl-1H-pyrazol-4-yr)phenyl]ethyl}pyrimidin-4-amine;
5-{3-[(1S)-1-{[2-methy I-6-(1-melhyl-1H-indol-6-yl)pyrimidin-4-yl]amino}ethyl]phenyl}pyrimidin-2-amine;
2-methyl-6-(3-methyl-1-benzofuian-5-yl)-N-{(1 S 1-[5-(I-methyl-1H-pyrazol-5-yl)pyridin-3-yl]ethyl}pyrimidin-4-amine;
2-methyl-6-(3-methyl-I-benzoturan-5-yl)-N-{(1S)-1-[5-(1H-pyrazol-4-yl)pyridin-3-yljethyl}pyrimidin-4-amine;
(2S)-3-[(5-{3-[(IS)—I-{[2-methyl-6-(3-methyl-I-benzofuran-5-yl)pyrimidin-4-yl]an no}elhyl]phenyl}pyrimidin-2-yl)amino]propane-I,2-diol;
2-methyl-N-{1-[3-(1-methyl-1H-pyrazol-4-yl)phenyl]ethyl}-6-[4-(trifliioromethyl)phenyl]pyrimidin-4-aniine;
6-(2,3-dihydro-1,4-benzodioxin-6-yl)-2-niethyl-N-{I-[3-(I-methyl-1H-pyrazol-4-yl)phenyl]ethyl}pyrimidin-4-amine;
2-methyl-6-(3-methyl-1-benzofuran-5-yl)-N-{(1S)-1-[3-(1H-pyrazol-1-yl)phenyl]elhyl}pyrimidin-4-amine;
2-methyl-6-(3-methyl-I-benzoliran-5-yl)-N—{(IS)—I-[3-(2H-la2,3-triazol-2-yl)phenyl]etliyl}pynmidin-4-amine;
6-(I-benzothien-5-yl)-2-methyl-N-{I-[3-(I-methyl-1H-pyrazol-4-yl)phenyl]ethyl}pyrimidin-4-amine;
N′-{5-[3-(I-{[6-(I,3-benzothiazol-6-yl)-2-methylpyrimidin-4-yl]amino}ethyl)phenyrlpyrimidin-2-yl}-NaN-dimethylethane-1,2-diamine;
2-methyl-6-(3-methyl-1-benzofuran-5-yl)-N-{(1S)-1-[3-(1H-tetrazol-1-yl)phenyl]ethyl}pyrimidin-4-amine;
2-methyl-6-(3-methyl-1-benzof′uran-5-yl)-N-{(1)-I-[3-(1H-tetrazol-1-yl)phenyl]ethyl}pyri m id i n-4-amine;
N—{(IS)—I-[3-(2-{4-[2-(dimethylamino)ethyl]piperazin-I-yl}pyrimidin-5-yl)phenyl]ethyl}-2-methyl-6-(3-methyl-I-benzofuran-5-yl)pyrimidin-4-amine; 2-[4 5-{3-[(IS)—I-{[2-methyl-6-(3-methyl-I-benzoftiran-5-yl)pyrimidin-4-yl]amino}ethyl]phenyl}pyrimidin-2-yl)piperazin-I-yl]ethanol;
2-methyl-6-(3-methy!-I-benzofuran-5-yl)-N—{(I S)-I-[3-(2-{4-[(I-methyl-1H-imidazol-2-yl)methyl]piperazin-1-yl}pyrimidm\hat{ }
2-methyl-6-(3-methyl-1-benzofuran-5-yl)-N—{(IS)—I-[3-(2-{4-[2-(methyloxy)ethyl]piperazin-yl}pyrimidin-5-yl)phenyl]ethyl}pyrimidin-4-amine;
2-({2-[4-(5-{3-[(IS)-I-{[2-methyl-6-(3-methyl-I-benzofuran-5-yl)pyrimidin-4-yl]amino}ethyl]phenyl}pyrimidin-2-yl)piperazin-1-yl]ethyl}oxy)et anoH
2-meth I-6-(3-meth]-1-benzof\hat{ }iran-5-I)- -[(IS)-1-(3-{2-[4-(2-Ino holin-4-ylethyl)piperazin-1-yl]pyrimidin-5-yl}phenyl)ethyl]pyrimidin-4-amine;
N-[(IS)—I-(3-bromophcnyl)ethyl]-2-methyl-6-(3-methyl-1-benzothien-5-yl)pyrimidin-4-amine;
2-methyl-6-(3-methyl-I-benzotliien-5-yl)-N—{(IS)—I-[3-(I-methyl-IH-pyrazol-4-yl)phenyl]ethyl}pyrimidin-4-amine;
N—{(IS)—I-[3-(5-aminopyridin-3-yl)phenyl]ethyl}-2-methyl-6-(3-methyl-I-benzothien-5-yl)pyrimidin-4-amine;
N—{(IS)—I-[3-(6-fluoropyridin-3-yl)phenyl]ethyl}-2-methyl-6-(3-methyl-1-benzolhien-5-yl)pyrimidin-4-amine;
N—{(IS)—I-[3-(5-fluoropyridin-3-yl)phenyl]ethy]}-2-methyl-6-(3-methyl-]-benzothien-5-yl)pyrimidin-4-amine;
5-{3-[(IS)-{[6-(3-chloiO-I-benzofuran-5-yl)-2-methylpyrimidin-4-yl]amino}ethyl]phenyl}pyrimidin-2-amine;
ethyl 5-[3-(1-{[6-(I,3-benzothiazol-6-yl)-2-methylpyrimidin-4-yl]amino}ethyl)phenyl]pyridinc-3-carboxylat′e;
6-(1,3-benzothiazol-6-yl)-N-(I-{3-[6-(dimethylamiiio)pyridin-3-yl]phenyl}ethyl)-2-methylpyrimidin-4-amine;
N-[(IS)—I-(3-bromophenyl)ethyl]-6-(3-ethyl-1-benzofuran-5-yl)-2-methylpyrimidin-4-amine;
5-{3-[(IS)-{[6-(,3-benzothiazol-6-yl)-2-methylpyrimidin-4-yl]amino}ethyl]phenyl}pyrimidin-2-amine;
6-(1,3-benzothiazol-6-yl)-N—{(IS)—I-13-(5-fluoropyridin-3-yl)phenyl]ethyl}-2-methylpyrimiclin-4-amine;
6-(1,3-benzothiazol-6-yl)-2-methyl-N—{(IS)—I-[3-(6-methy]pyridin-3-yl)phenyl]ethyl}pyrimidin-4-amine; 5-{3-[(IS)-1-{[6-(3-ethyl-I-benzoliran-5-yl)-2-melhylpyrimidin-4-yl]amino}ethyl]phenyl}pyrimidin-2-amine;
6-(1,3-benzothiazol-6-yl)-2-met yl-N—{(IS)—I-[3-(5-methylpyridin-3-yl)phenyl]ethyl}pyrimidin-4-amine;
6-(1,3-benzothiazol-6-yl)-N—{(IS)—I-[3-(6-fluoro-5-methylpyridin-3-yl)phenyl]ethyl}-2-methylpyrimidin-4-amine;
6-(1,3-benzothiazol-6-yl)-N—{(IS)—I-[3-(6-fluoropyridin-3-yl)phenyl]ethyl}-2-methylpyrimidin-4-amine;
-{(IS)—I-[3-(5-fluorapyridin-3-yl)phenyl]ethyl}-2-methyl-6-(3-methyl-I-benzofuran-5-yl)pyrimidin-4-amine;
6-(7-fluoro-I-benzoluran-5-yl)-2-methyl-N-{I-[3-(1-methyl-1H-pyrazol-4-yl)phenyl]ethyl}pyrimidin-4-amine;
N—{(IS)—I-[3-(2-chloropyrimidin-5-yl)pheny!]ethyl}-2-methyl-6-(3-methyl-1-benzofuran-5-yl)pyrimidin-4-amine;
6-(1,3-benzothiazol-6-yl)-N—{(IS)—I-[3-(2-chloropyrimidin-5-yl)phenyl]elhyl}-2-methylpyrimidin-4-amine;
N—{(IS)—I-[3-(6-fluoropyridin-3-yl)phenyl]elhyl}-2-methyl-6-(3-methyl-I-benzofuran-5-yl)pyrimidin-4-amine;
5-{3-[(IS)-1-{[6-(I,3-benzothiazol-6-yl)-2-methylpyrimidin-4-yl]amino}ethyl]phenyl}-N-methylpyrimidin-2-amine;
N-methyl-5-{3-[(IS)-1-{[2-methyl-6-(3-metliyl-1-benzoruran-5-yl)pyrimidin-4-yl]amino}ethyl]phenyl}pyrimidin-2-aniine;
5-{3-[(1S)-1-{[6-(7-fluoro-1-benzoiuran-5-yl)-2-methylpyrimidin-4-yl]amino}ethyl]phenyl}pyrimidin-2-aniine;
5-{3-[(IS)-1-{[6-(I,3-benzothiazol-6-yl)-2-methylpyrimidin-4-ylJamino}ethyl]phenyl}-N,N-dimethylpyninidin-2-amine;
N,N-dimethyl-5-{3-(IS)-1-{[2-methyl-6-(3-methyl-1-benzofuran-5-yl)pyrimidin-4-yl]amino}ethyl]phenyl}pyrimidin-2-amine;
N-ethyl-5-{3-[(IS)-1-{[2-melhyl-6-(3-methyl-1-benzofuran-5-yl)pyrimidin-4-yl]amino}ethyl]phenyl}pyrimidin-2-amine;
5-{3-[(1S)-1-{[6-(1,3-benzolhiazol-6-y I)-2-methy Ipyrimidin-4-yl]amino}ethyl]phenyl}-N-ethylpyrimidin-2-amine;
2-methyl-6-(3-methyl-I-benzofiiran-5-yl)-N—{(IS)—I-[3-(2-morpholin-4-ylpyrimidin-5-yl)phenyl]ethyl}pynmidin-4-amine: 2-methyl-6-(3-methyl-1-benzofuran-5-yl)-N—{(1S)-1-[3-(2-piperazin-1-ylpyrimidin-5-yl)phenyljethyl}pyrimidin-4-amine;
N—{(IS)—I-[3-(5-aminopyndiiv3-yl)phenyl]ethyl}-2-methyl-6-(3-methyl-I-benzouran-5-yl)pyrimidin-4-amine;
2-[(5-{3-[(IS)-]-{[2-melhyl-6-(3-niethyl-1-benzofiiran-5-yl)pyrimidin-4-yl]amino}ethyl]phenyl}pyrimidin-2-yl)amino]ethanol;
N,N-diethyl-5-{3-[(IS)-1-{[2-methyl-6-(3iiethyl-1-benzofuran-5-yl)pyrimiyl]amino}ethyl]phenyl}pynmidin-2-amine;
N—{(IS)—I-[3-(5-chloropyridin-3-yl)phenyl]ethyl}-2-methyl-6-(3-methyl-I-benzo(uran-5-yl)pyrimidin-4-amine;
6-(1,3-benzothiazol-6-yl)-N—{(IS)—I-[3-(5-chioropyridin-3-yl)phenyl]ethyl}-2-methylpyrimidin-4-amine:
5-{3-[(IS)-{[6-(13-benzothiazol-6->)-2-methylpyrimidin-4-yl]amino}ethyl]phenyl}-N,N-diethylpyrimidin-2-amine;
6-(1,3-benzolhiazol-6-yl)-N-[(IS)-]-{3 2-(4-ethylpiperazin-1-yl)pyrimidin-5-yl]phenyl}ethyl]-2-methylpyrimidin-4-amine;
I-(5-{3-[(IS)-1-{j6-(],3-benzot iazol-6-yl)-2-melhylpyrimidin-4-yl]amino}ethyl]phenyl}pyrirnidin-2-yl)piperidin-4-ol;
N-[(IS)-1-{3-[2-(4-ethylpiperazin-I-yl)pyrimidin-5-yl]phenyl}ethyl]-2-methyl-6-methyl-I-benzofiiran-5-yl)pyrimidin-4-amine;
1-(5-{3-[(IS)-1-{[2 methyl-6-(3-methyl-1-benzofuran-5-yl)pynmidin-4-yl]amino}ethyl]plienyl}pyrimidin-2-yl)pipei″idin-4-ol;
I-(5-{3-[(IS)-1-{2-methyl-6-(3-methyl-I-benzoiuran-5-yl)pynmidin-4-yl]amino}ethyl]phenyl}pyniTiidin-2-YI)pyrrolidin-3-ol;
N-(I-methylethyl)-5-{3-[(1S)-1-{[2-melhyl-6-(3-methyl-1-benzofiiran-5-yl)pyrimidin-4-yl]amino}ethyl]phenyl}pyriinidin-2-aniine:
[I-(5-{3-[(IS)-1-{[2-methyl-6-(3 iielhyl-I-benzofuran-5-yl)pyrimidin-4-yl]amino}ethyl]phenyl}pyrimidin-2-yl)piperidin-4-yl]methanol;
N-[(IS)-1-{3-[2-(4 1 uoropiperidin-1-yl)pyrimidin-5-yl]phenyl}ethyl]-2-methyl-6-(3-methyl-1-benzofuran-5-yl)pyrimidin-4-amine;
N furan-2-y!methyl)-5-{3-[(IS)-1-{[2 iiethyl-6-(3\hat{ }ethyl-1-benzofuran-5-yl)pyrirnidin-4-yl]amino}ethyl]phenyl}pynmidin-2-amine;
N-(furan-3-ylmethyl)-5-i3-[(IS)-1-{[2-methyl-6-(3\hat{ }Iiethyl-I-benzofuran-5-yl)pyrimidin-4-yl]amino}ethyl]phenyl}pyrimidin-2-ainine; 6-(7-fluoro-3-methyl-1-benzofuran-5-yl)-2-methyl-N-{1-[3-(I-methyl-IH-pyrazol-4-yl)phenyl]ethyl}pyrimidin-4-amine:
(5-{3-[(IS)-1-{[6-(13-benzothiazol-6-yr)-2-methylpyrimidin-4-yl]amino}ethyl]p enyl}pyridin-2-yl)met anol;
2-methyl-6-(3-metliyl-1-benzoinan-5-yl)-N—{(IS)—I-[3-(4-methyl-3,4-dihydro-2H-pyrido[3,2-b][1,4]oxazin-7-yl)phenyl]elliyl}pyrimidin-4-ainine;
(5-{3-[(1S)-1-{[6-(1,3-benzolhiazol-6-yl)-2-methy lpyrimidin-4-yl]amino}ethyl]phenyl}pyridin-3-yl)methanol;
N—{(IS)—I-[3-(5-amino-6-chloropyridin-3-yl)phenyl]ethyl}-2-methyl-6-(3-methyl-I-benzofuran-5-yl)pyrimidin-4-amine;
6-(7-fluoro-3-met yl-1-benzofuran-5-yl)-2-methyl-N-{(1S)-1-[3-(1-methyl-1H-pyrazol-4-yl)phenyl]ethyl}pyrimidin-4-amine;
6-(3 4-difluorophenyl)-2-methyl-{1-[3-(I-methyl-1H-pyrazol-4-yl)phenyl]ethyl}pyrimidin-4-amine;
(5-{3-[(IS)-1-{[2-methyl-6-(3-methyl-1-benzofuran-5-yl)pynmidin-4-yljamino}ethyl]phenyl}pyridin-3-yl)methanol;
N—{(IS)—I-[3-(5-aminopyndin-3-yl)phenyl]etliyl}-6-(7-fluoro-3-methyl-1-benzofuran-5-yl)-2-metliylpyrimidin-4-amine;
6-(7-IluoiO-3-methyl-1-benzofuran-5-yl)- -{(IS)-1-[3-(5-iluoiOpyridin-3-y)phenyl]ethyl}-2-methy]pyrimidin-4-amine;
6-(4-chloro-3,5-difluoiOphenyl)-2-methyl-N-{I-[3-(I-methyl-IH-pyrazol-4-yl)phenyl]ethyl}pyrimidin-4-amine;
2-methyl-N-{I-[3-(I-methyl-1H-pyrazol-4-yl)phenyl]ethyl}-6-(3,4,5-ti′iiluorophenyl)pyrimidin-4-amine;
N—{(IS)—I-[3-(5-amino-6-chloropyridin-3-yl)phenyl]ethyl}-6-(7-fluoiO-3-methyl-1-benzofuran-5-yl)-2-methylpyrimidin-4-amine;
5-{3-[(IS)-1-{[6-(3,4-dif]uorophenyl)-2-melhylpyrimidin-4-yl]amino}ethyl]phenyl}pyrimidin-2-amine;
5-{3-[(IS)-1-{[6-(4-chloro-3?5-ditluoiOphenyl)-2-methylpyrimidin-4-y 1] amino} ethy I]phenyl} py ri m idin-2-amine;
I-(5-{3-[(IS)-1-{[2-methyl-6-(3-methy]-1-bcnzofuran-5-yl)pyrimidin-4-ylJamino}ethyl]phenyl}pyridin-3-yl)ethanone;
6-(7-fluoro-3-methyl-1-benzofuran-5-yl)-2-methyl-N-{1-[5-(IH-pyrazol-4-yl)pyridin-3-yl]ethyl}pyiimidin-4-amine; N-{I-[5-(I-ethyl-1H-pyrazol-4-yl)pyridin-3-yl]ethyl}-6-(7-nuoro-3-methyl-1-benzofuran-5-yl)-2-met y]pyrimidin-4-amine:
6-(7-fluoro-3-melhyl-I-benzofuran-5-yl)-N—{(1S)-1-[3-(6-tluoropyridin-3-yl)phenyl]ethyi}-2-methylpyi-imidin-4-ainine;
ethyl 5-{3-[(IS)-1-{[2-methyl-6-(3-methyl-1-benzofuran-5-yl)pyrimidin-4-yl]amino}ethyl]phenyl}pyridine-3-carboxylate;
3-[(5-{3-[(IS)-1-{[6-(7-nuoro-3-methyl-1-benzofuran-5-yl)-2-methylpyrimidin-4-yl]amino}ethyl]phenyl}pyrimidin-2-yl)amino]propane-1,2-diol;
1-(5-{3-[(1S)-1-{[6-(7-fluoiO-3-methyl-1-benzofuran-5-yl)-2-methylpyrimidin-4-yl]amino}ethyl]phenyl}pyrimidin-2-yl)piperidin-4-ol;
1-(5-{3-[(IS)-1-{[2-methyl-6-(3-methyl-1-benzofuran-5-yl)pyrimidin-4-yl]amino}ethyl]phenyl}pyridiii-3-yl)elhanol;
2-(5-{3-[(IS)-1-{[2-methyl-6-(3-met yl-1-benzofuran-5-yl)pyrimidin-4-yl]amino}ethyl]phenyl}pyridin-3-yl)propan-2-ol;
6-(7-fluoro-3-methyl-1-benzofuran-5-yl)-2-methyl-N-{1-[5-(1-methyl-1H-pyrazol-4-yl)pyridin-3-yl]ethyl}pyrimidin-4-amine;
5-[5-(1-{[6-(7-nuoro-iiiethyl-I-benzofuran-5-yl)-2-methylpyrimidin-4-yl (amino}ethyl)pyridin-3-yl]pynmidin-2-amine;
5-{3 (IS)-1-{[6-(7-fluoiO-3-metliyl-1-benzofuran-5-yl)-2-methylpyrimidin-4-yl]amino}ethyl]phenyl}pyrimidin-2-amine;
2-methyl-6-(3-methyl-1-benzofuran-5-yl)-N-[(1S)-1-(3-pyridin-3-ylphenyl)ethyl]pyrimidin-4-amine;
6-(1,3-benzothiazol-6-yl)-N-{1-[3-(2-nuoropyridin-3-yl)phenyl]ethyl}-2-methylpyrimidin-4-amine;
6-(I.3-benzothiazol-6-yl)-2-methyl-N-{1-[3-(2-methylpyridin-4-yl)phenyl]ethyl} pyrimidin-4-amine;
N-[(1S)-1-{3-[6-(dimelhylamino)pyridin-3-y I]phenyl} elhyl]-2-methyl-6-(3-methyl-1-benzofiran-5-yl)pyrimidin-4-amine;
6-(1,3-benzothiazol-6-y)-2-methyl-N-{1-[3-(6-piperazin-1-ylpyridin-3-yl)phenyl]ethyl}pyrimidin-4-amine;
2-methyl-6-(3-niethyl-I-benzoluran-5-yl)-N-{I-[5-(I-methyl-1H-pyrazol-4-yl)pyridin-3-yl]ethyl}pyrimidin-4-amine;
2-methyl-6-(3-methyl-I-benzofiiran-5-yl)-N—{(IS)—I-[3-(6-methylpyridin-3-yl)phenyl]ethyl}pyrimidin-4-amine: 2-methyl-6-(3-niethyl-I-benzoiiiran-5-yl)-N—{(IS)—I-[3-(6-piperazin-ylpyridi yl)phenyl]ethyl}pyrimidin-4-amine;
2-methyl-6-(3-melhyl-I-benzofuran-5-yl)-N—{(IS)-1-[3-(5-methylpyridin-3-yl)phenyl]ethyl}pyrimidin-4-amine;
N—{(IS)—I-[3-(6-nuoro-5-methylpyridin-3-yl)phenyl]ethyl}-2-niethyl-6-(3-methyl-I-benzofuran-5-yl)pyrimidin-4-amine;
6-(1-benzothiazol-6-yl)-N—{(IS)—I-[3-(2-fluoropyridin-4-yl)phenyl]ethyl}-2-methylpyrimidin-4-amine;
5-[4-(I-{[2-methyl-6-(3-methyl-1-benzofuran-5-yl)pyrimidin-4-yljamino}ethyl)pyridin-2-yjpyrimidin-2-amine;
N—{(IS)—I-[3-(6-chloropyridin-3-yl)phenyl]ethy}-2-methyl-6-(3-methyl-I-benzofuian-5-yl)pyrimidin-4-amine;
2-methyl-N-[(I S)-1-{3-[6-(methylamino)pyridin-3-yl]phenyl}ethy]]-6-(3-methyl-I-benzofuran-5-yl)pyrimidin-4-amine;
2 methyl-6-(3\hat{ }Iietliyl-I-benzofuran-5-yl)-N—{(IS)—I-[3-(6-morpholin-4-ylpyndin-3-yl)phenyl]ethyl}pyrimidin-4-amine;
2-[(5-{3-[(]S)-1-{[2-methyl-6-(3-methy]-1-benzofuran-5-yl)pyrimidin-4-yl]amino}ethyl]phenyl}pyridin-2-yl)amino]ethanol;
6-(1:3-benzothiazol-6-yl)-N—{(IS)—I-[3-(6-chloi pyridin-3-yl)phenyl]ethyl}-2-methylpyrimidin-4-amine;
6-(I-benzofuran-5-yl)-2-methyl-N-{1-[3-(I-methyl-1H-pyrazol-4-yl)phenyl]ethyl}pyrimidin-4-amine;
2Mnethyl-6-(3-methyl-1-benzofuran-5-yl)-N—{(IR)-I-[2-(I-methyl-1H-pyrazol-4-yl)pyridiri-4-yl]ethyl}pyrimidin-4-amine;
2-methyl-6-(3-methyl-1-benzofuran-5-yl)-N-{(1S)-1-[2-(1-methyl-1H-pyrazol-4-yl)pyridin-4-yl]ethyl}pyrimidin-4-amine;
N-[(1S)-I-{3-[6-(ethylamino)pyridin-3-yl]phenyl}ethyl]-2-methyl-6-(3-methyl-I-benzofuran-5-yl)pynmidin-4-aminc;
N-[(IS)-1-{3-[6-(4-ethylpiperazin-I-yl)pyridin-3-yl]phenyl}ethyl]-2-methyl-6-(3-methyl-1-benzofuran-5-yl)pyrimidin-4-amine;
6-(1-benzothiazol-6-yl)-N-[(IS)-1-{3-[6-(4-ethylpiperazin-1-yl)pyridin-3-yl]phenyl}ethyl]-2-methylpyrimidin-4-amine;
5-[5-(1-{[2-methyl-6-(3-melhyl-1-benzo uran-5-yl)pyrimidin-4-yl]amino}ethyl)pyridin-3-yljpyriniidin-2-amine: 6-(I,3-benzothiazol-6-yl)-2-methyl-N-{(1S)-I-[3-(6-morpholin-4-ylpyridin-3-yl)phenyl]ethyl}pyrimidin-4-amine;
2-methyl-6-(3-methyl-1-benzofuran-5-yl)-N—{(IR)-I-[5-(I-methyl-1H-pyrazol-4-yl)pyridin-3-yl]ethyl}pyrimidin-4-amine;
I-(5-{3-[(IS)-1-{[2-methyl-6-(3-mctliyl-1-benzofuran-5-yl)pyriniidin-4-yl]amino}ethyl]phenyl}pyridin-2-yl)piperidin-4-ol;
6-(1,3-benzothiazol-6-yl)- -{(IS)-]-[3-(6-chloro-5-methylpyridin-3-yl)phenyl]ethyl}-2-methylpyrimidin-4-amine;
N—{(IS)—I-[3-(6-chloi-5-methylpyridin-3-yl)phenyl]ethyl}-2-methyl-6-(3-methyl-I-benzofuran-5-yl)pyrimidin-4-amine;
N-[1-(5-bromopyridin-3-yl)ethyl]-2-methyl-6-(3-metliyl-1-benzofuran-5-yl)pyrimidin-4-amine;
5,-(I-{[2-methyl-6-(3-metliyl-I-benzofuran-5-yl)pyrimidin-4-yl]ann′no}ethyl)-3 bipyridin-5-amine;
6-(4-chloro-3-fluorophenyl)-2-methyl-N-{1-[3-(I-methyl-1H-pyrazol-4-yl)phenyl]ethyl}pyrimidin-4-amine;
6-(3-t1 uorophenyl)-2-methyl-N-{(1S)-1-[3-(1-methyl-1H-pyrazol-4-yl)phenyl]ethyl}pyrimidin-4-amine;
5-{3-[(IS)—I-{[6-(4-chlorophenyl)-2-methylpyrimidin-4-yl]amino}ethyl]phenyl}pyrimidin-2-amine;
5-{3-[(IS)-{[6-(4-chloro-3-l:liiorophenyl)-2-methylpynmidin-4-yl]amino}ethyl]phenyl}pyrimidin-2-amine;
N—{(IS)—I-[3-(5-aminopyridin-3-yl)pl nyl]ethyl}-6-(4-chloiO-3-fluorophenyl)-2-methylpyrimidin-4-amine;
5-[5-(I-{[6-(4-chloiO-3-fluoroplienyl)-2-methylpyrimidin-4-yl]amino}ethyl)pyridin-3-yl]pyrimidin-2-amine;
6-(4-chloiO-3-fluorophenyl)-2-niethyl-N-{I-[5-(I-methyl-IH-pyrazol-4-yl)pyridin-3-yl]ethyl}pyrimidin-4-amine;
6-(4-chloro-3-fliiorophenyl)-N-{I-[5-(I-ethyl-IH-pyrazol-4-yl)pyridin-3-yl]ethyl}-2-methylpyrimidin-4-amine;
6-(4-chloro-3-nuoiOphenyl)-2-methyl-N-{(1S)-1-[3-(6-piperazin-I-ylpyridin-3-yl)phenyl]ethyl}pyrimidin-4-amine;
6-(4-chloro-3-fluorophenyl)-2-methyl-N—{(I S)-1-[3-(1-methyl-1H-pyrazol-4-yl)phenyl]ethyl}pyrimidin-4-amine; 6-(3-fluorophenyl)-2-methyl-N—{(I)-I-[3-(I-meihyl-1H-pyrazol-4-yl)phenyl]ethyl}pyrimidin-4-amine;
3-[(5-{3-[(IS)-{[6-(4-chloro-3-nuorophenyl)-2-methylpyrimidin-4-yl]amino}ethyl]phenyl}pyrimidin-2-yl)amino]pi pane-1,2-diol;
1-(5-{3-[(I S)-1-{[6-(4-chloro-3-fluorophenyl)-2-methylpyrimidin-4-yl]amino}ethyl]phenyl}pyrimidin-2-yl)piperidin-4-ol;
N—{(IS)—I-[3-(4-chloiOpyridin-3-yl)p enyl]etliyl}-2-methyl-6-(3-niethyl-1-benzofi.iran-5-yl)pyrimidin-4-amine;
6-(4-chloro-3-nuorophenyl)-N—{(IS)—I-[3-(I-ethyl-1H-pyrazol-4-y!)phenyl]ethy]}-2-met ylpyrimidin-4-amine;
5-{3-[(IS)-{[2-methyl-6-(3-methyl-1-benzofuran-5-yl)pyrimidin-4-yl]amino}ethyl]phenyl}pyridine-3-carboxaniide;
3-[2-methyl-6-({1-[3-(I-methyl-1H-pyrazol-4-yl)phenyl]ethyl}amino)pyrimidin-4-yl]phenol;
6-(2,3-dihydro-1-benzofuraii-5-yl)-2-methyl-N-{1-[3-(1-methyl-1H-pyrazol-4-yl)phenyl]ethyl}pyrimidin-4-amine;
6-[2-fliioiO-3-(methyloxy)phenyj-2-methyl-N-{Il-[3-(I-methyl-1H-pyrazol-4-yl)phenyl]ethyl}pyrimidin-4-amine;
6-(1,3-benzothiazol-6-yl)-2-methyl-N-(1-{3-[5-(methyloxy)pyridin-3-yl]phenyl}ethyl)pyrimidin-4-amine;
6-(1,3-benzothiazol-6-yl)-N—{I-[3-(2-(luoi pynmidin-5-yl)phenyl]ethyl}-2-melhylpyi′imidin-4-amine;
5-[3-(I-{[6-(I,3-benzothiazol-6-yl)-2-methylpyrimidin-4-yl]amino}ethyl)phenyl]pyrimidine-2-carbonitrile;
6-(1,3-benzothiazol-6-yl)-N-{1-[3-(6-IluoiO-2-methylpyridin-3-yl)phenyl]ethyl}-2-methylpyrimidin-4-amine;
5-[3-(I-{[6-(1,3-benzothiazol-6-yl)-2-methylpyrimidin-4-yl]amino}ethyl)phenyl]pyridine-3-carbonitrile;
6-(1,3-benzothiazol-6-yl)-N-{1-[3-(4-chlorapyridin-3-yl)phenyl]ethyl}-2-methylpyrimidin-4-amine;
6-(13-benzothiazol-6-yl)-N-{1-[3-(2)-dimethylpyridin-3-yl)phenyl]ethyl}-2-methylpyrimidin-4-amine;
4-[3-(I-{[6-(1,3-benzothiazol-6-yl)-2-methylpynmidin-4-yl]amino}ethyl)phenyl]pyrimidin-2-amine; 6-(1,3-benzothiazol-6-yl)-2-methyl-N-{(1S)-1-[3-(2-piperidin-1-ylpyrimidin-5-yl)phenyl]ethyl}pyrimidin-4-amine;
N′-(5-{3-[(IS)-1-{[6-(1,3-benzothiazol-6-yl)-2-methylpyrimidin-4-yl]amino}ethyl]phenyl}pyrimidin-2-yl)-N,N-diiTietliylpropane-1,3-diamine;
-[(IS)-1-{3-[2-(4-acetylpiperazin-1-yl)pynmidin-5-yl]phenyl}ethyl]-2-methyl-6-(3-methyl-1-benzofuraii-5-yl)pyrimidin-4-amine;
5-{3-[(IS)-1-{[2-melhyl-6-(3-melhyl-1-benzoluran-5-yl)pyrimidin-4-yl]amino}ethyl]pheny]}-N-(tetraliycli furan-2-ylmethyl)pyrimidin-2-ainine;
6-(3-amino-4-chlorophenyl)-2-methyl-N-{I-13-(1-methyl-1H-pyrazol-4-yl)phenyl]ethyl}pyrimidin-4-amine;
6-(4-chloro-3-methylphenyl)-2-methyl-N-{1-[3-(1-methyl-1H-pyrazol-4-yl)phenyl]ethyl}pyrimidin-4-amine;
2-fluoro-4-[2-methyl-6-({1-[3-(I-methyl-1H-pyrazol-4-yl)phenyl]ethyl}amino)pyrimidin-4-yl]benzamide;
6-(1,3-benzothiazol-5-yl)-2-methyl-N-{I-[3-(I-methyl-1H-pyrazol-4-yl)phenyl]ethyl}pyrimidin-4-amine;
N-[(IS)-1-{3-[2-(4-acetylpiperazin-1-yl)pyrimidin-5-yl]phenyl}ethyl]-2-methyl-6-(3-meth I-1-benzothien-5-yl)pyrimidin-4-amine;
N—{(IS)-[3-(2-{4-[2-(dimethylamino)ethyl]piperazin-1-yl}pyrimidin-5-yl)phenyl]ethyl}-2-methyl-6-(3-methyl-1-benzothien-5-yl)pyrimidin-4-amine;
5-{3-[(IS)-1-{[2-methyl-6-(3-methyl-1-benzofuran-5-yl)pyrimidin-4-yl]amino}ethyl]phenyl}pyridine-3-carbonitrile;
2-methyl-6-(3-methyl-1-benzofuran-5-yl)-N-[(1:
S)-1-(6′-methyl-3,3′-bipyridin-5-yl)ethyl]pyrimidin-4-amine;
2-methyl-6-(3-methyl-1-benzofuran-5-yl)-N-{(1S)—I-[5-(I-methyl-1H-pyrazol-4-yl)pyndin-3-yl]ethyl}pyrimidin-4-amiiie
5-{3-[(IS)-1-{[2-methyl-6-(3-methyl-1-benzothien-5-yl)pyrimidin-4-yl]amino}ethyl]phenyl}pyrimidin-2-amine;
N—{(IS)—I-[3-(5-aminopyridin-3-yl)phenyl]ethyl}-6-(I,3-benzothiazol-6-yl)-2-methylpyrimidin-4-amine; and
5-{3-[(1S)-1-{[2-methyl-6-(3-methyl-1-benzofuran-5-yl)pyrimidin-4-yl]amino}ethyl]phenyl}pyrimidin-2-ol;
(S)-5-(3-(I-(6-(7-fluoro-3-methylbenzof ran-5-yl)-2-methylpyrimidin-4-ylamino)ethyl)phenyl)pyrimidin-2-amine; (S)-2-methyl-N—(I-(5-(1-methyl-1H-pyrazol-4-y I)pyridin-3-yl)ethyl)-6-(3-methylbenzofuran-5-yl)pyrimidin-4-amine;
(S)—N-(]-(3-(5-aminopyriclin-3 yl)phenyl)ethyl)-6-(4-chloro-3-fluol phenyl)-2-methylpyrimidin-4-amine;
(S)-5-(3-(I-(2-methyl-6-(3-methylbenzot′uran-5-yl)pynmidin-4-ylamino)ethyl)phenyl)pyrimidin-2-amine;
N—(I-(5-(I-ethyl-1H-pyrazol-4-yl)pyridin-3-yl)ethyl)-6-(7-fluoro-3-melhylbenzol\iran-5-yl)-2-metliylpyrimidin-4-amine;
5-(3-fluoiO-5-(1-(2-metliyl-6-(3-melhylbenzofuran-5-yl)pyrimidin-4-ylamino)ethyl)phenyl)pyrimidin-2-amine;
(S)-5-(3-(I-(2-meth\-6-(3-methylbenzo[b]thiophen-5-yl)pyrimidin-4-ylamino)ethyl)phenyl)pyrimidin-2-amine; or
(S)—N—(I-(3-(5-aminopyridin-3-yl)phenyl)ethyl)-2-methyl-6-(3-methylbenzo[b]thiophen-5-yl)pyrimidin-4-amine.

42. A compound of items 1-35 which is:
(56-(Benzo[\hat{ }thiazol-5-yl)-N-(I-cyclohexylethyl)-2-mediylpyrimidin-4-aniine;
2-methyl-6-[3-(methylo>y)phenyl]-N—{(IR)-I-[3-(methyloxy)phenyl]elhyl}pyrimidin-4-amine;
2-methyl-6-[3-(methyloxy)phenyl]-N—{(IS)—I-[3-(methyloxy)phenyl]ethyl}pynmid 4-amine;
2-methyl-6-[3-(methyloxy)phenyl]-N—{(IS)—I-[4-(methyloxy)phenyl]ethyl}pyrimid 4-amine;
N,N-diethyl-2-{[3-(I-{2-methyl-6-(I-methyl-1H-indol-6-yl)pyrimidin-4-yl]amino}ethyl)phenyl]oxy}acetamide;
N,N-dimethyl-2-{[3-(I-{[2-methyl-6-(I-melhyl-1H-indol-6-yl)pyrimidin-4-yl]amino}ethyl)phenyl]oxy}acetamide:
3-(1-{[2-methyl-6-(I-methyl-l H-indol-6-yl)pyrimidin-4-yl]amino}ethyl)benzenesulfonamide;
N-ethyl-2-{[3-(1-{[2-methyl-6-(I-methyl-rH-indol-6-yl)pyrimidin-4-yl]amino}ethyl)phenyl]oxy}acctamide;
N-(cyanomethyl)-3-(]-{[2-methyl-6-(I-methyl-1H-indol-6-yl)pyrimidin-4-yl]amino}ethyl)benzenesulfonamide; 2-methyl-6-(I-methyl-IH-indol-6-yl)-N-(I-{3-[(2-morpholin-4-yl-2-oxoethyl)oxy]phenyl}ethyl)pyrimidin-4-amine;
4-{[3-(I-{[2-methyl-6-(1-methyl-1H-indol-6-yl)pyrimidin-4-yl]amino}ethyl)phenyl]oxy}butanoic acid;
2-methyl-6-(I-methyl-1H-indol-6-yl)- -{(IS)—I-[3-(methyloxy)phenyl]ethyl}pyi″imidin-4-amine;
2-methyl-6-(I-methyl-1H-indol-6-yl)-N-[(IS)-1-pheiiylethyl]pyriniidin-4-amine; 6-[2-chlo!O-3-(methyloxy)phenyl]-2-methyl-N—{(IS)-1-[3-(melhyloxy)phenyl]ethyl}pyrimidin-4-amine;
6-(1,3-benzodioxol-5-yl)-2-methyl-N—{(IS)—I-[3-(methyloxy)phenyl]ethyl}pyrimidin-4-amine;
3-(1-{[2-methyl-6-(I-methyl-1H-indol-6-yl)pyrimidiii-4-yl]amino}ethyl)phenol; 6-(IH-indol-5-yl)-2-melhyl-N-{(IS)—I-|3-(methyloxy)phenyl]ethyl}pyrimidin-4-amine;
ethyl N-({[3-(I-{[2-methyl-6-(I-methyl-1H-indol-6-yl)pyrimidin-4-yrjamino}ethyl)phenyrjoxy}acetyl)glycinate;
N-[(IS)—I-(4-nuorophenyl)ethyl]-2-methyl-6-(I-methyl-1H-indol-6-yl)pyrimidin-4-amine;
2-methyl-6-(1-methyl-1H-indol-6-yl)-N-[(1S)-1-(4-methylphenyl)ethyl]pyrimidin-4-amine;
ethyl 4-{[3-(1-{[2-methyl-6-(I-methyl-1H-indol-6-yl)pyrimidin-4-yl]amino}ethyl)phenyl]oxy}butanoate;
6-(3-fluorophenyl)-2-methyl-N—{(IS)—I-[3-(methyloxy)phenyl]ethyl}pyrimidin-4-amine;
2-({3-[I-({6-[2-chloi-3-(methyloxy)phenyl]-2-methylpynmidin-4-yl}amino)ethyl]phenyl}oxy)- -methylacelamide;
2-{[3-(1-{[6-(1,3-benzodioxol-5-yl)-2-methylpyrimidin-4-yl]amino}ethyl)phenyl]oxy}-N-methylacetamide;
N-(cyanomethyl)-3-1 1-({2-methyl-6-[3-(methyloxy)phenyl]pyrimidin-4-yl}amino)ethyl]benzenesulfonamide;
({3-[I-({2-methyl-6-[3-(methyloxy)phenyl]pyrimidin-4-yl}amino)ethyl]phenyl}oxy)acetonitrile;
3-[1-({6-[2-chloiO-3-(melhyloxy)phenyl]-2-methylpyrimidin-4-yl}amino)ethyl]benzenesulfOnamide;
6-[2-fluoro-3-(methyloxy)phenyl]-2-methyl-N—{(IS)—I-[3-(melhyloxy)phenyl]ethyl}pyrimidin-4-amine;
N-methyl-2-({3-[I-({2-methyl-6-[3-(methyloxy)phenyl]pyrimidin-4-yl} amino)ethy 1] phenyl}oxy)acetamide;
N-[I-(3-biOmophenyl)ethyl]-6-[2-chloro-3-(methyloxy)phenyl]-2-methylpyrimidin-4-amine;
methyl ({3-[I-({2-methyl-6-[3-(methyloxy)phenyl]pyrimidin-4-yl} amino)ethyl]pheny 1} oxy)acetate;
methyl N-{3-[I-({2-methyl-6-[3-(methyloxy)phenyl]pyrimidin-4-yl}amino)ethyjphenyl}glycinate;
N-[(1S)-1-(4-chlorophenyl)ethyl]-2-methyl-6-(1-methyl-1H-indol-6-yl)pyrimidin-4-amine;
3-[I-({2-methyl-6-[3-(methyloxy)phenyl]pyrimidin-4-yl}amino)ethyl]benzenesuliOnamide;
6-(I-elhyl-1H-inclol-6-yl)-2-methyl-N—{(I S)-1-[3-(methyloxy)phenyl]ethyl}pyrimidin-4-amine;
methyl N-({3-[1-({2-methyl-6-[3-(methyloxy)phenyl]pyrimidin-4-yl}amino)ethyl]phenyl}sulfonyl)glycinate;
2-methyl-6-(1-methyl-1H-indol-5-yl)-N—{(1S)-1-3-(melhyloxy)phenyl]ethyl}pyrimidin-4-amine;
3-[1-({2-methyl-6-[3-(metlvloxy)phenyl]pyrimidin-4-yl}amino)ethyl]phenol;
1,1-dimethylethyl (cyanomethyl)({3-[1-({2-methyl-6-[3-(methyloxy)phenyl]pyrimidin-4-yl}amino)ethyl]phenyl}sulfonyl)carbamate;
2-melhyl-6-[3-(methyloxy)phenyl]-N-{(1S)—I-[3-(trifluoromethyl)phenyl]ethyl}pyrimidin-4-amine;
2-melhyl-N—{(IS)—I-[3-(methyloxy)phenyl]elhyl}-6-{3-[(triiluoromethyl)oxy]phenyl}pyrimidin-4-amine;
2-methyl-N—{(IS)—I-[3-(methyloxy)phenyl]elhyl}-6-[3,4:5-tris(methyloxy)phenyl]pyrimidin-4-amine;
methyl N-{[(I,I-dimethylethyl)oxy]carbonyl}-N-({3-[I-({2-methyl-6-[3-(methyloxy)phenyl]pyrimidin-4-yl}amino)elhyl]phenyl}sulfonyl)glycinate;
6-[2-chloro-5-(methyloxy)phenyl]-2-mehyl-N—{(IS)—I-[3-(mclhyloxy)phenyl]elhyl}pynmidin-4-amine: 6-(IH-ind I-6-yl)-2-met yl-N—{(IS)—I-[3-(methyloxy)phenyl]ethyl}pyrimidin-4-amine;
N-{3-[I-({2-melhyl-6-[3-(methyloxy)phenyl]pyrimidin-4-yl}amino)ethyl]phenyl} glycine
N-({3-[I-({2-methyl-6-[3-(methyloxy)phenyl]pyrimidin-4-yl}amino)ethyl]phenyl}sulfonyl)glycine:
N-metliyl-2-{[3-(1-{[2-methyl-6-(1-methyl-1H-indol-6-yl)pyrimidin-4-yl]amino}ethyl)phenyl]oxy}acetamide;
2-methyl-6-(3-methyl-1-benzofuran-5-yl)-N—{(IS)—I-[3-(methyloxy)phenyl]ethyl}pyrimidin-4-amine;
6-(I-benzoiuran-5-yl)-2-metliyl- -{(IS)—I-[3-(methyloxy)phenyl]ethyl}pyrirnidin-4-amine;
6-(I-benzothien-5-yl)-2-methyl-N—{(IS)—I-[3-(methyloxy)phenyl]ethyl}pyrimidin-4-amine;
2-methyl-N—{(IS)—I-[3-(methyloxy)phenyl]ethyl}-6-(4-methylphenyl)pyrimi amine;
6-(4-chloi phenyl)-2-methyl-N-{(1S)-I-[3-(methyloxy)phenyl]ethyl} pyrimidin-4-amine;
6-(3-chlorophenyl)-2-methy-N-{(1S)-1-[3-(methyloxy)phenyl]ethyl} pyrimidin-4-amine;
6-(2-fluorophenyl)-2-methyl-N—{(IS)—I-[3-(metliyloxy)phenyl]ethyl}pyrimidin-4-amine;
6-(4-fluoiOphenyl)-2-methyl-N—{(IS)—I-[3-(melhyloxy)phenyl]ethyl}pyrimidin-4-amine;
2-methyl-N—{(IS)—I-[3-(methyloxy)phenyl]elhyl}-6-(2-methylphenyl)pyrimidin-4-amine;
6-(13-benzoxazol-6-yl)-2-methyl-N—{(IS)—I-[3-(methyloxy)phenyl]ethyl}pyrimidin-4-amine;
6-(I,3-benzothiazol-6-yl)-2-methyl-N—{(I R)-1-[3-(methyloxy)phenylJethyl}pyrimidin-4-amine;
6-(I H-indol-4-yl)-2-methyl-N-{(1S)-1-[3-(methyloxy)phenyl]ethyl}pyrimidin-4-amine;
6-(1H-indol-7-yl)-2-methyl-{(1S)-1-[3-(methyloxy)phenyl]ethy} pyrimidin-4-amine; 6-(2-c lorop enyl)-2-rnethyl-N—{(IS)—I-[3-(methyloxy)phenyl]ethyl}pyrimidin-4-amine;
2-methy I-6-(1-methyl-1H-benzimidazol-6-yl)-N-{(1S)-1-[3-(methyloxy)phenyl]etliyl}pyrimidin-4-amine;
6-[3-(dimethylamino)phenyl]-2-methyi-N—{(IS)—I-[3-(methy Ioxy)pheny I]ethyl} pyrimidin-4-amine;
6-(IH-indazol-5-yi)-2-methyl-N—{(IS)—I-[3-(methyloxy)phenyl]ethyl}pyrimidin-4-aminc;
6-[4-(dimethylamino)phenyl]-2-methyl-N—{(I S)-1-[3-(methyloxy)phenyl]ethyl}pyiimidin-4-amine;
6-(I3-benzothiazol-6-yl)-2-metliyl-N—{(IS)—I-[3-(methyloxy)phenyl]ethyl}pyrimidin-4-amine;
2-methyl-N—{(IS)—I-[3-(methy]oxy)phenyl]ethyl}-6-phenylpyrimidin-4-amine;
6-(3-elhylphenyl)-2-methyl-N—{(IS)—I-[3-(methy]oxy)phenyl]ethyl}pyrimidin-4-amine;
2-methy I-N-{(IS)-1-[3-(methyloxy)phenyl]ethyl}-6-(3-methylphenyl)pyrimidin-4-amine:
2-methyl-6-pyridin-4-yl-N-[(IR)-1,2,3,4-tetrahydronaphthalen-1-yl]pyrimidin-4-amine;
2-methyl-N—{(IS)—I-[3-(methyloxy)phenyl]ethyl}-6-pyridin-3-ylpyrimidin-4-amine;
2-methyl-N—{(IS)—I-[3-(methyloxy)phenyl]ethyl}-6-pyridin-4-ylpyrimidin-4-amine;
N-[I-(3-bromophenyl)ethyl]-2-methyl-6-[3-(methyloxy)phenyl]pyrimidin-4-amine;
N-[I-(3-{[(1,3-dimethyl-1H-pyrazol-5-yl)methyl]oxy}phenyl)ethyl]-2-methyl-6-(1-methyl-1H-indol-6-yl)pyrimidin-4-amine;
6-(L3-benzothiazol-6-yl)- -[(1S)-1-(3-{[(1,3-dimethyl-1H-pyrazol-5-yl)methyl]oxy}phenyl)elhyl]-2-methylpyrimidin-4-amine;
2-fluoro-5-(I-{[2-methyl-6-(3-methy-I-benzofuran-5-yl)pyrimidin-4-yl]amino}ethyl)phenol
N-{-[4-fluoro-3-(I-methyl-1H-pyrazol-4-yl)phenyjethyl}-2-methyl-6-(3-methyl-1-benzoiiran-5-yl)pyrimidin-4-amine;
6-[2-chloro-3-(methyloxy)phenyl]-N-[I-(3-{[(1,3-dimethyl-1H-pyrazol-5-yl)methyl]oxy}phenyl)ethyrj-2-methylpyrimidin-4-amine;
2-methyl-6-[4-methyl-3-(methyloxy)phenylJ-N—{(IS)—I-[3-(methyloxy)phenyl]ethy 1} pynmidin-4-amine;
3-[(IS)—I-{[6-(1,3-benzothiazol-6-yl)-2-niethylpynmidin-4-yl]amino}et yl]phenol
N-[I-(3-{[(I)3-dimethyl-II-1-pyiazol-5-yl)methyl]oxy}pheny])ethyl]-2-methyl-6-[4-methyl-3-(methyloxy)phenyl]pyrimidin-4-amine;
6-(1,3-benzothiazol-6-yl)-2-melhyl-N-(I-{3-[(IH-pyrazol-3-ylmetliyl)oxy]phenyl}ethyl)pyrimidiii-4-amine;
3-(I-{[6-(1,3-benzothiazol-6-yl)-2-methylpyrimidin-4-yl]amino}ethyl)-N-(cyanomethyl)benzeiiesulfonamide;
6-(I-benzothiazol-6-yl)-N-(I-{3-[(isoxazol-3-ylmethyl)oxy]phenyl}ethyl)-2-methylpyrimidin-4-amine;
3-(I-{[6-(1,3-benzotliiazol-6-yl)-2 nethylpynmidin-4-yl]amino}ethyl)-N-but-2-yn-1-ylbenzenesulfonamide;
2-methyl-6-[4-methyl-3-(methyloxy)phenyl]-N-[I-(3-{[(I-methyl-IH-pyrazol-3-yl)methyl]oxy}phenyl)ethyl]pyrimidin-4-amine;
6-(I 3-benzothiazol-6-yl)-N-[1-(3-{[(I,3-dimethyl-1H-pyrazol-5-yl)methyl]oxy}-4-ttuorophenyl)elhyl]-2-melhylpyrimidin-4-amine;
2-({3-[I-({2-methyl-6-[4-methyl-3-(methyloxy)phenyl]pyrimidin-4-yl}amino)ethyl]phenyl}oxy)acetamide;
3-(I-{[6-(1,3-benzolhiazol-6-yl)-2-methylpyrimidin-4-yl]amino}ethyl)-N-prop-2-yn-1-ylbenzenesulfonaniide;
6-(1,3-benzothiazol-6-yl)-N—{I-[4-nuoiO-3-(I-methyl-1H-pyrazol-4-yl)phenyl]ethyl}-2-methylpyrimidin-4-amine;
{[3-(I-{[6-(1,3-benzothiazol-6-yl)-2-methylpyrimidin-4-yl]amino}ethyl)phenyl]oxy}acetonitrile
N-(I-{3-[(2-azepan-I-yl-2-oxoethyl)oxy]plienyl}ethyl)-6-(1,3-benzothiazol-6-yl)-2-methylpyrimidin-4-amine;
2-{[3-(I-{[6-(1,3-benzothiazol-6-yl)-2-melhylpyrimidin-4-yl]amino}ethyl)phenyl]oxy}-N-(I-methylethyl)acetamide;
6-(2,3-dihydro-1-benzo<jran-5-yl)-2-melhyl-N—{(IS)—I-[3-(methyloxy)plienyl]ethyl}pyrimidin-4-amine;
6-(1,3-benzothiazol-6-yl)-2-methyl-N-[I-(3-{[2-(2-methylaziridin-1-yl)-2-oxoetliyl]oxy}phenyl)ethyl]pyrimidin-4-amine;
2-{[3-(]-{[6-(1,3-benzothiazol-6-yl)-2-methylpyrimidin-4-yl]amino}ethyl)phenyl]oxy}-N,N-dimethylacetamide; 3-(1-{[6-(1,3-benzothiazol-6-yl)-2-methylpyrimidin-4-yl]amino}ethyl)benzenesulfonamide;
6-(K3-benzothiazol-6-y])-2-methyl-N-[I-(3-{[(5-methylisoxazol-3-yl)methyl]oxy}phenyl)ethyl]pyrimidin-4-amine;
{[3-(1-{[2-methyl-6-(1-methyl-1H-indol-6-yl)pyrimidin-4-yl]amino}ethyl)phenyl]oxy}acelonitrile;
6-(1,3-benzothiazol-6-yl)-2-methyl-N-{I-3-(prop-2-yn-1-y loxy)phenyl]elhy 1} pyri m idi n-4-am i ne:
-{1-[2-f1 uoro-3-(methyloxy)phenyl]ethyl}-2-methyl-6-(3-methyl-1-benzofuran-5-yl)pyrimidin-4-amine;
2-cliloiO-5-(1-{[2-methyl-6-(3-methyl-1-benzofuran-5-yl)pyrimidin-4-yl]amino}ethyl)phenol;
3-[1-({2-rnethyl-6-[4-methyl-3-(methyloxy)phenyl]pyrimidin-4-yl}amino)ethyl]phenol
6-(1,3-benzothiazol-6-yl)-2-methy]-N-(I-{3-[(2-oxo-2-piperidin-1-ylethyl)oxy]phenyl}elhyl)pyrimidin-4-amine;
N-(1-{3-[(2-azetidin-1-yl-2-oxoelhyl)oxy]phenyl}ethyl)-6-(1,3-benzothiazol-6-yl)-2-methylpyrimidin-4-amine;
2-{[3-(1-{[6-(1,3-benzothiazol-6-yl)-2-methylpyrimidin-4-y]amino}ethyl)phenylloxy}-N-(2-IiydroxypiOpyl)acetamide;
3-(I-{[6-(1,3-benzothiazol-6-yl)-2-methylpynmidin-4-yl]amino}ethyl)-N-(2-hydroxyethyl)benzenesullOnamide;
6-(L3-benzothiazol-6-yl)-N-[I-(5-{[(I)₃-dimethyl-1H-pyrazol-5-yl)methyl]oxy}-2-fluorophenyl)ethyl]-2-methylpyrimidin-4-amine;
3-(I-{[6-(1,3-benzothiazol-6-yl)-2-methylpyiimidin-4-yl]amino}ethyl)-N-[2-(methyloxy)elhyl]benzenesulfonamide;
3-[(IR)-1-{[6-(1,3-benzothiazol-6-yl)-2-methylpyrimidin-4-yrjamino}ethyl]phenol
6-(1,3-benzothiazol-6-i)-2-methyl-N-(I-{3-[(2-morpholin-4-yl-2-oxoethyl)oxy]phenyl}ethyl)pyi′imidin-4-amine;
3-(]-{[6-(1,3-benzothiazol-6-yl)-2-methylpyrimidin-4-yl]amino}ethy)-4-fluoiophenol
3-[I-({6-[2-fluoro-3-(niethyloxy)phenyl]-2-methylpyriniidin-4-yl}amino)ethyl]phenol N,N-diethyi-2-({3-[I-({6-[2-fluoro-3-(methyloxy)phenyl]-2-methylpyrimidin-4-yl}amino)ethyl]phenyl}oxy)acetamide; 6-(1,3-benzothiazol-6-yl)-N-(IR)-I-(3-{[(1,3-Iimelliyl-1H-pyrazol-5-yl)inethyl]oxy}phenyl)ethyl]-2-methylpyrimidin-4-amine;
6-(13-benzothiazol-6-yl)-2-methyl-N-(I-{3-[(2-oxo-2-pyiTolidin-1-ylethyl)oxy]phenyl}ethyl)pyrimidin-4-amine;
2-methyl-6-(1-methyl-1H-indol-6-yl)-N-[1-(3-{[2-oxo-2-(4-pyridin-2-ylpiperazin-1-yl)elhyl]oxy}phenyl)ethyl]pyrimidin-4-amine;
methyl 1-({[3-(1-{[2-methy I-6-(1-methyl-1H-indol-6-yl)pyrimidin-4-yl]amino}ethyl)phcnyl]oxy}acelyl)pipei′idine-4-carboxylate;
2-{[3-(I-{[6-(1,3-benzothiazol-6-yl)-2-methylpyrimidin-4-yl]amino}ethyl)phenyljoxy}-N-methylacetamide;
2-{[3-(I-{[6-(1,3-benzothiazol-6-yl)-2-methylpyrimidiiv4-yl]amino}ethyl)phenyl]oxy}-N-ethylacetamide;
3-(I-{[6-(I 3-benzothiazol-6-yl)-2-methylpyrimidin-4-yl]amino}ethyl)phenol {[3-(1-{[2-methyl-6-(1-methyl-1H-indol-6-yl)pyrimidin-4-yl]amino}ethyl)phenyl]oxy}acetic acid;
4-{[3-(1-{[6-(1,3-benzolhiazol-6-yl)-2-methylpyrimidin-4-yl]amino}ethyl)phenyl]oxy}butanoic acid;
ethyl 4-{[3-(1-{[6-(1,3-be zothiazol-6-yl)-2-methy lpyrimidin-4-yl]amino}ethyl)phenyljoxy}butanoate;
1,1-dimethylethyl (3S)-3-({[6-(1,3-benzothiazol-6-yl)-2-methylpyrimidin-4-yl]amino}methyl)piperidine-1-carboxylate;
2-{[3-(1-{[6-(I;3-beiizolhiazol-6-yl)-2-methylpyriniidin-4-yl]amino}ethyl)phenyl]oxy}-N,N-diethylacelamide;
6-(4-chloropheny I)-2-methyl-N-{1-[3-(1-methyl-1I-I-pyrazol-4-yl)phenyl]ethyl}pyrimidin-4-amine;
6-(3-fluoiOphenyl)-2-methyl-N-{1-[3-(I-methyl-1H-pyrazol-4-yl)phenyl]ethyl}pyrimidin-4-amine;
2-methyl-6-[3-(methyloxy)phenyl]-N-{I-[3-(I-methyl-1H-pyrazol-4-yl)phenyl]ethyl}pyrimidin-4-aniine;
6-(1H-ind6I-5-yl)-2-melhyl-N-{1-[3-(1-methyl-1H-pyrazol-4-yl)plienyl]ethyl}pyi′imidin-4-amine;
2-methyl-6-(3-methylphenyl)-N-{1-[3-(I-methyl-1H-pyrazol-4-yl)phenyl]ethyl}pyrimidin-4-amine; 6-(1,3-benzothiazol-6-yl)-N-[(2-chloro-6-nuorophenyl)methyl]-2-melhylpyrimidin-4-amine;
2-met yl-N-{I-[3-(I-methyl-IH-pyrazol-4-yl)phenyl]ethyl}-6-phenylpyrimidin-4-amine;
2-methyl-6-[4-(methyloxy)phenyl]-N-{1-[3-(1-methyl-1H-pyrazol-4-yl)phenyl]ethyl}pyrimidin-4-amine;
6-(2,4-dichlorophenyl)-2-methyl-N-{I-[3-(I-methyl-1H-pyrazol-4-yl)phenyl]ethyl}pyrimidin-4-amine;
2-methyl-6-(2-methylphenyl)-N-{1-[3-(1-methyl-1H-pyrazol-4-yl)phenyl]ethyl}pyrimidin-4-amine;
6-(3-chloiO-4-fluorophenyl)-2-methyl-N-{I-[3-(I-melhyl-1H-pyrazol-4-yl)phenyl]ethyl}pyrimidin-4-amine;
N-i\hat{ }-CI\hat{ }-CI\hat{ }-benzothiazol-e-y \hat{ }-methylpyrimidin\hat{ }-yl]amino}methyl)piperidin-1-yl)-2-oxoethyl}-N-methylbenzamide;
2-methyl-N-{1-[3-(I-methyl-1H-pyrazol-4-yl)phenyl]ethyl}-6-naphthalen-2-ylpyrimidin-4-amine;
6-(1,3-benzothiazol-6-yl)-2-melhyl-N-(I-pyridin-3-ylelhyl)pyrimidin-4-amine;
2-methyl-6-(3-methyl-1-benzofuran-5-y I)-N-{1-[3-(1H-tetrazol-1-yl)phenyl]elhy 1} pyrimidin-4-amine;
2-methyl-6-(3-methyl-1-benzofuian-5-yl)-N-{1-3-(4H-1,2,4-triazol-4-yl)phenyl]ethyl}pyrimidin-4-amine;
2-methyl-6-(3-methyl-I-benzofiiran-5-yl)-N-[I-(3-niti phenyl)ethyl]pyriniidin-4-amine;
6-(I-benzothiazol-6-yl)-2-methyl-N-{I-[3-(IH-1,2;3-triazol-I-yl)plienyl]ethyl}pyrimidin-4-amine;
N-[3-(1-{12-methyl-6-(3-methyl-1-benzofuran-5-yl)pyrimidin-4-yl]amino}ethyl)phenyl]piOp-2-enamide;
2-methyl-6-(1-methyl-1H-indol-6-yl)-N-{1-[3-(I-methyl-1H-pyrazol-4-yl)phenyl]ethyl} pyrimidin-4-amine;
N-[I-(3-aminophenyl)ethyl]-2-methyl-6-(3-methyl-1-benzofuran-5-yl)pyrimidin-4-amine;
6-(I:3-benzothiazol-6-yl)-2-methyl-N-{1-[3-(5-methyl-1H-tetrazol-1-yl)phenyl]ethyl}pynmidin-4-amine; 6-(1,3-benzothiazol-6-yl)-2-melhyl-N-{1-[3-(I H-tetrazol-1-yl)phenyl]elhyl}pyrimidin-4-amine;
6-(1,3-benzothiazol-6-yl)-2-met yl-N-{1-[3-(4H-1,2,4-′triazol-4-yl)phenyljethyl}pyrimidin-4-amine;
3-(1-{[6-(1,3-benzothiazol-6-yl)-2 nethylpyrimidin-4-yl]amino}ethyl)benzonitrile
2-{[3-(1-{[6-(1,3-benzothiazol-6-yl)-2-methylpyrimidin-4-yl]amino}ethyl)phenyl]oxy}-N,N-diethylpropanamide;
2-{[3-(1-{[6-(I:3-benzothiazol-6-yl)-2-methylpyrimidin-4-yl]amiiio}ethyl)phenyl]oxy}-2-methylpropanamide;
e-CI\hat{ }-benzothiazol\hat{ }-y \hat{ }-melhyl-N-O- -CS-methyl-I\hat{ }\hat{ }-oxadiazol\hat{ }-yl)phenyl]ethyl}pyrimidin-4-amine;
2-{[3-(I-{[6-(I,3-benzothiazol-6-yl)-2-methylpyrimidin-4-yl]amino}ethyl)pheiiyl]oxy}piOpanamide;
3-(]-{[6-(1,3-bcnzothiazol-6-yl)-2-methylpyrimidin-4-yl]amino}ethyl)benzenecarboximidamidc;
N-[3-(I-{[6-(] 3-benzothiazol-6-yl)-2Hiiethylpyrimidin-4-yl]amino}ethyl)phenyl]-1H-pyrazole-5-carboxamide;
N-[3-(I-{[6-(1,3-benzothiazol-6-yl)-2-methylpy
methyl-1H-pyrazole-3-carboxamide;
N-[3-(I-{[6-(1,3-benzothiazol-6-yl)-2-methylpyrimidin-4-yl]amino}ethyl)phenyl]dicarbonimidic diamide;
N-[3-(I-{[6-(I.3-benzothiazol-6-yl)-2-methylpyrimidin-4-yl]amino}ethyl)phenyl]formamide;
I-[3-(I-{[6-(1,3-beiizothiazol-6-yl)-2′-methylpyrimidin-4-yl]amino}ethyl)phenyl]urea;
N-[3-(I-{[6-(1,3-benzothiazol-6-yl)-2-methylpyrimidin-4-yl]amino}ethyl)phe methyl-IH-imidazole-4-sulibnamide;
6-(1,3-benzothiazol-6-yl)-2-methyl-N-{1-[3-(2H-tetrazol-5-yl)phenyrlethyl}pyrimidin-4-amine;
1,1-dimethylethyl (2-{[3-(I-{[6-(K3-benzothiazol-6-yl)-2-methylpynmidin-4-yl]amino}ethyl)phenyl]amino}-2-oxoethyl)methylcarbamate;
3-(I-{[6-(1,3-benzot″hiazol-6-yl)-2-methylpyrimidin-4-yl]amino}ethyl)-N′-hydroxybenzenecarboximidamide; 2-methyl-6-(3-methyl-1-benzofuran-5-yl)-N-[(IS)-1-(3-pyrimidin-5-ylphenyl)ethyl]pyrimidin-4-amine;
5-{3-[(IS)-1-{[2-melhyl-6-(3-methyl-I-bcnzofuran-5-yl)pyrimidin-4-yl]amino}ethyl]phenyl}pyrimidin-2-amine;
N-{(1S)-1-[3-(6-aminopyiidin-3-yl)phenyl]ethyl}-2-inethyl-6-(3-methyl-1-benzofuran-5-yl)pyrimidin-4-amine:
ethyl (4-{3-[(IS)-1-{[2-methyl-6-(3-methyl-1-benzofuran-5-yl)pyrimidin-4-yl]amino}ethyjphenyl}-1H-pyrazol-1-yl)aceta(e;
2-methyl-1-{[3-(1-{[2-methyl-6-(3-methyl-1-benzof uran-5-yl)pyrimidin-4-yl]amino}ethyl)phenyl]oxy}propan-2-ol;
1-{[3-(1-{[2-methyl-6-(3-methyl-1-benzofuran-5-yl)pyrimidin-4-yl]amino}ethyl)pheny]oxy}pi″opaii-2-one:
6-(3-ethyl-1-benzol nan-5-yl)-2 iiethyl-N-[(IR)-1,2,3,4-tetrahydronaphthalen-I-yl]pyrimidin-4-amine;
(4-{3-[(1S)-1-{[2-methyl-6-(3-methyl-1-benzofuran-5-yl)pyrimidin-4-yl]amino}ethyl]phenyl}-1H-pyrazol-1-yl)acetic acid;
6-(I,3-benzothiazol-6-yl)-2-methyl-N-I′(1S)—I-(3-pyrimidin-5-ylphenyl)ethyl]pyrimidin-4-amine;
2-methyl-6-(3-methyl-I-benzofuiai>5-yl)-N-[I-(3-{[(5-methyl-1,2,4-oxadiazol-3-yl)methyl]oxy}phenyl)ethyl]pyrimidin-4-amine;
5-[3-(1-{[6-(1,3-benzothiazol-6-yl)-2-methylpyrimidin-4-y]amino}ethyl)phenyl]pyrimidin-2-amine;
ethyl (4-{3-[(IS)-1-{[6-(1,3-benzolhiazol-6-yl)-2-methylpyrimidin-4-yl]amino}ethyjphenyl}-1H-pyrazol-1-yl)acetate;
6-(7-Π-1-benzofuran-5-yl)-2-methyl-N—{(I S)—I-[3-(methyloxy)phenyl]ethyl}pyrimidin-4-amine;
2-methyl-6-(3-methyl-1-benzofuran-5-yl)-N-[I-(3-methylphenyl)ethyl]pyrimidin-4-amine;
6-(1,3-benzothiazol-6-yl)-N-[I-(3-{[(I-ethylpiperidin-3-yl)methyl]oxy}phenyl)ethyl]-2-methylpyrimidin-4-amine;
N-{I-[3-(6-aminopyridin-3-yl)phenyl]elhyl}-6-(1,3-benzothiazol-6-Y)-2-methylpyrimidin-4-amine;
6-(1,3-benzothiazol-6-yl)-2-methyl-[I-(3-{[(I-methylpiperidin-3-y)methyl]oxy}phenyl)ethyl]pyrimidin-4-amine; N-[I-(3-{[(1,3-dimethyl-H-I-pyrazol-5-yl)methyl]oxy}phenyl)etliyl]-2-methyl-6-(1-methyl-1H-indol-2-yl)pyrimidiii-4-amine;
2-methyl-6-(3-methyl-1-benzofuran-5-yl)-N-[(I S)-1-(4-methylplienyl)ethyl]pyrimidin-4-amine;
N-[I-(3-{[(1,3-dimethyl-1H-pyrazol-5-yl)methyl]oxy}phenyl)ethyl]-6-(3-ethyl-1-benzofuran-5-yl)-2-methylpynmidin-4-amine;
6-(1,3-benzothiazol-6-yl)-2-methyl-N-(1-{3-[(piperidin-3-y methyl)oxy]phenyl}ethyl)pyrimidin-4-amine;
1-{[3-(1-{[6-(1,3-benzotliiazol-6-yl)-2-methylpyrimidin-4-yl]amino}ethyl)phenyl]oxy}propan-2-oiie;
6-(1,3-benzothiazol-6-y])-2-methyl-N-{1-[3-(2-methyl-1,3-thiazol-4-yl)phenyl]ethyl}pyrimidin-4-amine;
6-(1,3-benzothiazol-6-yl)-N-[1-(5-{f(1,3-dimethyl-1H-pyrazol-5-yl)rnethyl]oxy}pyridin-3-yl)ethyl]-2-methylpynmidin-4-amine;
6-(3-ethyl-I-benzoiiiaii-5-yl)-2-methyl-N—{(IS)—I-[3-(methyloxy)phenyl]ethyl}pyrimidin-4-amine;
2-methyl-6-(3-methyl-I-benzotiran-5-yl)-N—{(IR)-I-[3-(I-methy-1H-pyrazol-4-yl)phenyl]ethyl}pyrimidin-4-amine;
1-{[3-(1-{[6-(1,3-benzothiazol-6-yl)-2-methylpyrimidin-4-ylJamino}ethyl)phenyl]oxy}propan-2-ol;
1-{[3-(1-{[6-(1,3-benzothiazol-6-yl)-2-methylpyrimidin-4-yl]amino}ethyl)phenyl]oxy}-2-methylpiOpan-2-ol;
N-[I-(2-fluorophenyl)ethyl]-2-nietliyl-6-(3-methyl-I-benzoi′uran-5-yl)pyrimidiiv4-amine;
3-(1-{[6-(3-ethyl-1-benzofuran-5-yl)-2-methylpyrimidin-4-yl]amino}ethyl)pte
2-methyl-6-(I-methyl-1H-indol-2-yl)-N—{(IS)—I-[3-(methyloxy)phenyl]ethyl}pyrimidin-4-amine;
6-(I-benzothiazol-6-yl)-N-[I-(2-chloropyridin-4-yl)ethyl]-2-methylpyriniidin-4-amine;
6-(1,3-benzothiazol-6-y I)-2-methyl-N-[1-(3-{[(5-methyl-1,2,4-oxadiazol-3-yl)metliyl]oxy}phenyl)ethyl]pyi′imidin-4-amine;
N-[I-(3-{[(I-acetylpiperidin-3-yl)methyl]oxy}phenyl)elhyl]-6-(1,3-benzothiazol-6-yl)-2-methylpyrimidin-4-amine; 2-{[3-(1-{[2-methyl-6-(I-methyl-1H-indol-2-yl)pyrimidin-4-yl]amino}ethyl)phenyl]oxy}acetamide;
6-(L3-benzothiazol-6-yl)-2-methyl-N-(1-{3-[(piperidin-4-ylniethyl)oxy]phenyl}ethyl)pyrimidin-4-amine:
2-methyl-6-(4-melhyl-3,4-dihydiO-2H-1,4-benzoxazin-6-yl)-N-{1-[3-(I-methyl-1H-pyrazol-4-yl)phenyl]ethyl} pyri midin-4-amine;
N-[1-(3-{[2-(4-acetylpiperazin-1-yl)ethyl]oxy} phenyl)ethyl]-6-(1,3-benzothiazol-6-yl)-2-methylpyrimidin-4-amine;
{4-[3-(I-{[6-(13-beiizothiazol-6-yl)-2-methylpynmidin-4-yl]amino}ethyl)phenyl]-IH-pyrazol-1-yl} acetic acid;
6-(3-elhyl-1-benzoruraii-5-yr)-2-methyl-N-{1-[3-(I-methyl-1H-pyrazol-4-yl)phenyl]ethyl}pyrimidin-4-amiiie;
2-{[3-(I-{[6-(1,3-benzolhiazol-6-yl)-2-methylpyrimidin-4-yl]amino}ethyl)phenyljoxy}-N-[2-(methyloxy)ethyl]acetamide;
2-{[3-(1-{[6-(1,3-benzothiazol-6-yl)-2-methylpyrimidin-4-yl]amino}ethyl)phenyl]oxy}-1-cyclopiOpylethanone;
2-{[3-(I-{[6-(1,3-benzothiazol-6-yl)-2-methylpyrimidin-4-yjamino}ethyl)phenyl]oxy}-N-phenylacetamide;
6-(I-benzofiiran-2-yl)-2-methyl-N—{(IS)—I-[3-(methyloxy)phenyl]ethyl}pyrimidin-4-amine;
1,1-dimethylethyl 3-({[3-(I-{[6-(1,3-benzothiazol-6-yl)-2-methylpyrimidin-4-yl]amino}ethyl)phenyl]oxy}melhyl)piperidine-1-carboxylate;
1,1-dimethylethyl 4-({[3-(I-{[6-(I,3-benzothiazol-6-yl)-2-methylpyrimidin-4-yl]amino}ethyl)phenyl]oxy}melhyl)piperidine-1-carboxylate;
6-(1,3-benzothiazol-6-yl)-2-methyl-N-(1-{3-[6-(methyloxy)pyridin-3-yl]phenyl}ethyl)pyrimidin-4-amine;
N;N-diethyl-2-{[3-(1-{[2-methyl-6-(4-methyl-3,4-dihydro-2H-1,4-benzoxazin-6-yl)pyrimidin-4-yl]amino}ethyl)phenyl]oxy}acetamide;
2-methyl-N—{(IS)—I-3-(melhyloxy)phenyl]ethyl}-6-(I-methy]-1H-pyn lo[3,2-b]pyridin-6-yl)pyrimidin-4-amine;
2-methyl-6-(3-methyl-I-benzorman-5-yl)-N-[I-(2-methylphenyl)ethyl]pyrimidin-4-amine;
6-(1,3-benzothiazol-6-yl)-2-methyl-N—{(I R)-I-[3-(1-methyl-1H-pyrazol-4-yl)phenyl]ethyl}pyrimidin-4-amine; (IS)-3-{[3-(I-{[6-(I,3-benzothiazol-6-yl)-2-melhylpynmidin-4-yl]amino}ethyl)phenyl]oxy}-1-phenylpropan-1-ol;
N,N liethyl-2-{[3-(1-{[2-methyl-6-(3 iiethyl-I-benzoiuran-5-yl)pyrirnidin-4-yl]amino}elhyl)phenyl]oxy}acetamide;
N-[I-(3-{[2-(IH-imidazol-1-yl)ethyl]oxy}pheny!)ethyl]-2-niethy]-6-(3-methyl-I-benzofuran-5-yl)pyrimidin-4-amine;
N—{(IS)—I-[3-(]-ethyl-IH\hat{ }yrazol-4-yl)phenyl]etliyl}-2-methyl-6-(3-methyl-I-benzofuran-5-yl)pyrimidin-4-amine;
2-methyl-6-(3-methyl-1-benzofuran-5-yl)-N-(1-{3-[(piperidin-3-ylmethyl)oxyjphenyl}ethyl)pyrimidin-4-amine;
2Miiethyl-6-(3nethyl-1-bciizotuOn-5-yl)-N-{(1S)-1-[3-(1-methyl-IH-pyrazol-5-yl)phenyl]ethyl}pyrimidin-4-amine;
2-{[3-(I-{[2-melhyl-6-(3-methyl-1-benzofiiran-5-yl)pyrimidin-4-yl]amino}ethyl)phenyl]oxy}ethanol;
2-methyl-6-(3 nelhyl-I-benzoiiran-5-yl)-N-[(IS)—I-{3-[(2-morpholin-4-yl-2-oxoethyl)oxy]phenyl}ethyl]pyrimidin-4-amine;
N-[I-(3-{[3-(dimethylamino)propyl]oxy}pheny])ethyl]-2-methyl-6-(3-metliyl-I-benzofuran-5-yl)pyrimidin-4-amine;
2-methyi-6-(3-methy I-1-benzofuran-5-yl)-N-[1-(3-{[(I-methyl-1H-pyrazol-3-yl)methyl]oxy}phenyl)ethyl]pyrimidin-4-amine;
3-(I-{[2-methyl-6-(3-methyl-]-benzofuran-5-yl)pyrimidin-4-yl]amino}ethyl)phenol-[(IS)-1-(3-biOmophenyl)ethyl]-2-methyl-6-(3-methyl-I-benzoluran-5-yl)pyrimidin-4-aniine;
2-methyl-6-(3-methyl-1-benzofuran-5-yl)-N—{(1S)-1-[3-(1H-pyrazol-4-yl)phenyl]etliyl}pyrimidin-4-amine;
3-[(IS)—I-{[2-methyl-6-(3-methyl-I-benzofuran-5-yl)pynmidin-4-yl]amino}ethyl]phenol
2-methyl-6-(3-methyl-1-benzofuran-5-yl)-N—{(1S)-I-[3-(I-methyl-1H-pyrrol-2-yl)phenyl]ethyl}pyrimidin-4-amine;
2-methyl-6-(3-methyl-I-benzofuran-5-yl)-N-[(IS)—I-phenylethyl]pyrimidin-4-aniine; 6-(I,3-benzothiazol-6-yl)-2-methyl-[I-(3-pyridin-3-ylphenyl)ethyl]pynmidin-4-amine;
6-(1,3-benzothiazol-6-yl)-2-methyl-N-{1l-[3-(I-methyl-1H-pyrazol-4-yl)phenyl]ethyl}pyrimidin-4-amine;
N-[(IS)—I-(4-fluoraphenyl)ethyl]-2-rnethyl-6-(3-melhyl-1-benzofuran-5-yl)pyrimidin-4-amine;
6-(I-benzofuran-5-yl)-N-[I-(3-{[(1,3-dimethyl-I H-pyrazol-5-yl)methyl]oxy}phenyl)el yl]-2-melhylpyrimidin-4-amine;
6-(1,3-benzothiazol-6-yl)-2-methyl-N-{(1S)-1-[3-(1-methyl-1H-pyrazol-5-yl)phenyl]ethyl}pyrimidin-4-amine;
-[ ]-(3-nuorophenyl)ethyl]-2-methyl-6-(3-methyl-1-benzofuran-5-yl)pyrimidin-4-amine;
6-(L3-benzothiazol-6-yl)-N—{(IS)—I-[3-(I-ethyl-1H-pyrazol-4-yl)phenyl]ethyl}-2-methylpyrimidin-4-amine;
2-methyl-6-(3-methyl-1-benzofuran-5-yl)-N—{(IS)—I-[3-(IH-pyrazol-5-yl)phenyl]ethyl}pyrimidin-4-amine;
2-methyl-6-(3-methyl-1-benzofuran-5-y -N-[(IR)-1-{3-[(2-morpholin-4-yl-2-oxoethyl)oxy]phenyl}ethyl]pyrimidin-4-amine; ′
3-[(IR)-{[2-methyl-6-(3-methyl-1-benzofuran-5-yl)pyrimidin-4-yl]amino}ethyl]phenol
6-(1,3-benzotliiazol-6-yl)-2-methyl-{(IS)—I-[3-(IH-pyrazol-4-yl)phenyl]ethyl}pyrimidin-4-amine;
6-(L3-benzothiazol-6-yl)-2-methyl-N-[I-(3-{[(methylsulfonyl)methyl]oxy}phenyl)ethyl]pyrimidin-4-amine;
[(2-iluoi-5-{2-methyl-6-[(IR)-1,2,3 etra\hat{ }\hat{ }yl}phenyl)amino]acetonitrile;
2-methyl-6-(3-methyl-1-benzofiiran-5-yl)-N—{(IR)-I-[3-(methyloxy)phenyl]etliyl}pyiimidin-4-amine;
2-(metliyloxy)-4-{2-methyl-6-[(IR)-I₁2,3,4-tetrahydronaphthalen-ylamino]pyrimidin-4-yl}benzamide;
6-(L3-benzothiazol-6-yl)-2-methyl-N-(I-{3-[(2-morpholin-4-ylethyl)oxy]plienyl}etliyl)pyrimidin-4-amine;
6-(1,3-benzothiazol-6-yl)-2-methyl-N—{(IS)—I-[3-(I-methyl-1H-pyrrol-2-yl)phenyl]ethyl}pyrimidin-4-amine;
6-(1,3-benzothiazol-6-yl)-2-methyl-N-[1-(3-{[2-(2-methyl-1H-imidazol-1-yl)ethyl]oxy}phenyl)ethyrjpyrimidin-4-amine;
6-(I-benzofiiran-5-yl)-2-methyl-N-|′I-(3-{[(I-methyl-1H-pyrazol-3-yl)methyl]oxy}phenyl)ethyl]pyiimidin-4-amine; N-[(IS)-1-biphenyl-3-ylethyl]-2-methyl-6-(3-methyl-1-benzofuran-5-yl)pyrimidin-4-amine;
4-{[3-(I-{[6-(1,3-benzothiazol-6-yl)-2-met ylpyrimidin-4-yl]amino}ethyl)phenyl]oxy}butan-i-ol;
6-(1,3-benzothia2ol-6-yl)-2-methyl-N-[I-(3-morpholin-4-ylphenyl)ethyl]pyrirnidin-4-amine;
3-{[3-(]-{[6-(1,3-benzolhiazol-6-yl)-2-methylpyrimidin-4-yllamino}ethyl)phenyl]oxy} propane-1,2-diol;
6-(I.3-benzolhiazol-6-yl)-N-[(IS)-1-biphenyl-3-yle iyl]-2-methylpyrimidin-4-amine;
2-{3-(I-{[6-(1,3-benzothiazol-6-yl)-2-methylpyrimidin-4-yl]amino}ethyl)phenyl]oxy}ethaiiol;
1-{[3-(I-{[6-(1,3-benzothiazol-6-yl)-2-methylpyrimidin-4-yl]amino}ethyl)phenyl]oxy}-3-Iluoropropan-2-ol;
6-(1,3-benzothiazol-6-yl)-N-[I-(3-bi niophenyl)ethyl]-2-methylpyrimidin-4-ami
3-{[3-(1-{[6-(1,3-benzothiazol-6-yl)-2-methylpyrimidin-4-yl]amino}elhyl)phenyl]oxy}propan-I-ok
4-{[3-(I-{[6-(1,3-benzothiazol-6-yl)-2-methylpyrimidin-4-yl]amino}elhyl)phenyl]oxy}-N,N-dimethylbutanainide:
6-(1,3-benzothiazol-6-yl)-N-[I-(3-{[3-(dielhylamino)propyl]oxy}phenyl)ethyl]-2-methylpyrimidin-4-amine;
6-(1,3-benzothiazol-6-yl)-2-methyl-N-[I-(3-{[2-(1H-pyrrol-I-yl)ethyl]oxy}phenyl)ethyl]pynmidin-4-amine;
6-(1,3-benzothiazol-6-yl)-2-methyl-N-(I-{3-[(3-morpholin-4-ylpropyl)oxy]phenyl}ethyl)pyrimidin-4-amine;
2-{[3-(I-{[6-(1,3-benzothiazol-6-yl)-2-methylpyrimidin-4-yl]amino}ethyl)phenyl]oxy}-N-(2-Iiydroxyethyl)acetamide;
2-{[3-(I-{[6-(1,3-benzothiazol-6-yl)-2-methylpyrimidin-4-yl]amino}ethyl)phenyl]oxy}-N-cyclopropylacetamide;
6-(1,3-benzothiazol-6-yl)-2-niethyl-N-{4-[3-(IH-pyrrol-2-yl)phenyl]ethyl}pyrimidin\hat{ }4-amine;
e-CI\hat{ }-benzothiazol\hat{ }-y \hat{ }-methyl-tl-iS-i\hat{ }-CIH-pyrazol-I-yl)ethyl]oxy}phenyl)ethyl]pyrimidin-4-amine;
N,N-diethyl-2-[(3-{1-[(2-metliyl-6-naphthalen-2-ylpyrimidin-4-yl)amino]ethyl}phenyr)oxy]acetamide; 6-(1,3-benzothiazol-6-yl)-2-met yl-N-(I-{3-[(3-pyrrolidin-I-ylpropyl)oxy]phenyl}elhyl)pyrimidin-4-amine;
6-(1,3-benzothiazol-6-yl)-N-[I-(3-{[3-(dimethylamino)propyl]oxy}phenyl)ethyl]-2-methylpyrimidin-4-amine;
6-(I,3-benzothiazol-6-yl)-2-methyl-N-{I-[2-(met yloxy)pyridin-4-yl]ethyl}pyrimidin-4-amine;
1,1-dimethylethyl 3-({[3-(I-{[2-methyl-6-(3-melhyl-1-benzoi′uran-5-yl)pyrimidin-4-yl]amino}ethyl)phenyl]oxy}methyl)piperidine-1-carboxylate;
2-{[3-(I-{[6-(1,3-benzothiazol-6-yl)-2-melhylpyrimidin-4-yl]amino}ethyl)phenyl]oxy}-N-cyclohexylacetamide;
6-(1,3-benzothiazol-6-yl)-N-[I-(3′-iliiorobiphenyl-3-yl)ethyl]-2-methylpyrimidin-4-amine;
methyl {[3-(I-{[6-(1,3-bcnzolhiazol-6-yl)-2-methylpyrimidin-4-yl]amino}ethyl)phenyl]oxy}acetate;
6-(L3-benzothiazol-6-yi)-2-methyl-N-[I-(3-{[2-(methylsullbnyl)cthyl]oxy}phenyl)ethyl]pyrimidin-4-amine;
6-(3-amino-4-methylphenyl)-2-methyl-N—{(I S)-1-[3-(methyloxy)phenyl jethyl} pyrimidin-4-amine;
2-methyl-N—{(IS)—I-[3-(methyloxy)phen>Jethyl}-6-quinolin-6-ylpyrimidin-4-amine:
{[3-(I-{[6-(1,3-benzothiazol-6-yl)-2-methylpyrimidin-4-yl]amino}ethyl)phenyl]oxy}acetic acid;
2-methyl-6-(3-methyl-1-benzofuran-5-yl)-N-{(1S)-1-[3-(1-methyl-1H-pyrazol-4-yl)phenyl]ethyl}pyrimidin-4-amine;
6-(1,3-benzothiazol-6-yl)-2-methyl-N—{(IS)-1-[3-(I-methyl-1H-pyrazol-4-yl)phenyl]ethyl}pyrimidin-4-amine;
N,N-dimethyl-2-({3-[(IS)-1-{[2-methyl-6-(3-methyl-1-benzofuran-5-yl)pyrimidin-4-yl]amino}ethyl]phenyl}oxy)acetamide;
N-[I-(3-{[2-(dimethylamino)elhyl]oxy}phenyl)ethyl]-2-methyl-6-(3-methyl-I-benzofuran-5-yl)pyrimidin-4-amine;
N,N-dimelhyl-2-({3-[(I R)-1-{[2-melhyl-6-(3-methyl-1-benzol iran-5-yl)pyrimidin-4-yl]amino}ethyl]phenyl}oxy)acelamide;
6-(1-benzothiazol-6-yl)-N-[I-(3-{[2-(dimelhylamino)elhyl]oxy}phenyl)ethyl]-2-methylpyrimidin-4-amine;
6-(2,5-dimethylphenyl)-2-methyl-N-{1-[3-(I-methyl-1H-pyrazol-4-yl)phenyl]ethyl}pyrimidin-4-amine;
6-(3:4-dichlorophenyl)-2-methyl-N-{I-[3-(I-melhyl-1H-pyrazol-4-yl)phenyl]eihyl}pyrtmidin-4-amine;
6-(4-ethylphenyl)-2-methyl-N-{I-13-(I-methyl-1H-pyrazol-4-yl)phenyl]ethyl}pyrimidin-4-amine;
6-(4-fluoiO-3-methylphenyl)-2-methyl-N-{1-[3-(1-methyl-1H-pyrazol-4-yl)phenyl]ethyl} pyrimidin-4-amine;
2-methyl-6-[4-(1-methylethyl)phenyl]-N-{I-[3-(I-methyl-IH-pyrazol-4-yl)phenyl]ethyl}pyrimidin-4-amine;
6-[2-fluoiO-4-(methyloxy)phen>iJ-2-melhyl-N-{I-[3-(I-methyl-1H-pyrazol-4-yl)phenyl]ethyl}pyrimidin-4-amine;
6-[3-(dimethylamino)phenyl]-2-methyl-N-{l-[3-(I-methyl-1H-pyrazol-4-yl)phenyl]ethyl} pyriniidin-4-amine;
6-(1,3-benzothiazol-6-yl)-N-[(IS)—I-(3-bromophenyl)ethyl]-2-methylpyrimidin-4-amine;
6-(1,3-benzothiazol-6-yl)-2-methyl-N—{(IS)—I-[3-(IH-pyrazol-5-yl)phenyl]elhyr}pyrimidin-4-amine:
(2E)-3-{3-[(IS)-1-{[6-(1:3-benzothiazol-6-yl)-2-methylpyrimidin-4-yl]amino}ethyl]phenyl}-N-elhylprop-2-enamide;
2-{[3-(1-{[6-(1,3-benzothiazol-6-y)-2-methylpyrimidin-4-yl]amino}ethyl)phenyl]oxy}-[2-(dimethylamino)ethyl]acetamide;
2-{[3-(1-{[6-(1,3-benzothiazol-6-y I)-2-methylpyrimidin-4-yl]amino}ethyl)phenyl]oxy}-N-propylaceiamide;
2-{[3-(1-{[6-(1,3-benzothiazol-6-yl)-2-methylpyrimidin-4-yl]amino}ethyl)phenyl]oxy}-N-ethyl-N-methylacetamide;
6-(1,3-beirzothiazol-6-yl)-2-methyl-N-[I-(3-nitiOphenyl)ethyl]pyrimidin-4-amine;
(2E)-3-{3-[(IS)-1-{[6-(1,3-benzothiazol-6-yl)-2-methy]pyrimidin-4-yl]amino}ethyl]pheny 1}-N-(1, 1-dimethylethyl)prop-2-enamide;
N-[I-(3-aminophenyl)ethyl]-6-(1,3-benzothiazol-6-yl)-2-methylpyrimidiiv4-amine;
2-({[3-(I-{[6-(1,3-benzothiazol-6-y)-2-methylpynmidin-4-yl]amino}ethyl)phenyl]oxy} methyl)-1,3-oxazole-4-carboxylic acid;
methyl 2-({[3-(I-{[6-(1,3-benzothiazol-6-yl)-2-methylpyrimidih-4-yl]amino}ethyl)phenyl]oxy} methyl)-1,3-oxazole-4-carboxylate; 6-(1,3-benzolhiazol-6-yl)-2-methyl-N-[(IS)-1-phenylethyl]pynmidin-4-amine;
2-methyl-6-(3-methyl-I-benzofiiran-5-yl)-N—{(IS)—I-[4-(methyloxy)phenyl]ethyl}pyrimidin-4-amine;
6-(1,3-benzothiazol-6-yl)-N-[1-(3-{[(1,3-dimethyl-1H-pyrazol-5-yl)methyl]oxy}phenyl)ethyl]-2-methylpyrimidin-4-amine;
N-[I-(3-iiran-3-ylphenyl)ethyl]-2-methyl-6-(3-niethyl-I-benzofuran-5-yl)pyrimidin-4-amine:
6-(1,3-benzothiazol-6-yl)-2-methyl-N-(1-{3-[(phenylmethyl)oxy]phenyl}ethyl)pyrimidin-4-amine;
2 nethyi-6-(3 methyl-I-benzofiiran-5-yl)-N—{]-[3-(3-thienyl)phenyl]ethyl}pyrimidin-4-amine;
6-(1,3-benzothiazol-6-yi)-2-methyl-N-(1-{3-[(pyndin-3-ylmethyl)oxy]phenyl}ethyl)pyrimidin-4-amine;
6-(1,3-benzothiazol-6-yl)-2-methyl-N-1-(3-{[(3-methylisoxazol-5-yl)methyl]oxy}plienyl)ethyl]pyrtmidin-4-amine;
6-(1,3-benzothiazol-6-yl)-2-methyl-N-[I-(3-{[(2-methyl-1,3-thiazol-4-yl)methyl]oxy}phenyl)ethyjpyrimidin-4-amine;
6-(1,3-benzothiazol-6-yl)-2-methyl-N-{1l-[3-(propyloxy)phenyl]ethy}pyrimidin-4-amine;
6-(1,3-benzothiazol-6-yl)- -{I-[3-(3,5-dimelhylisoxazol-4-yl)phenyl]ethyl}-2-methylpyrimidin-4-amine;
6-{2-melhyl-6-[(I \hat{ }-l\hat{ } \hat{ }-tetrahydi naphlhalen-1-ylarninoJpyrimidin\hat{ }-ylJ\hat{ }H-chromen-4-one;
6-(1,3-benzothiazol-6-yl)-2-methyl-N-[1-(3-{[(4-methylphenyl)melhyl]oxy}phenyl)ethyl]pyrimidin-4-amine;
6-(1,3-benzothiazol-6-yl)-N-[I-(3-furan-3-ylphenyl)ethyl]-2-methylpyrimidin-4-amine;
6-(1:3-benzothiazol-6-yi)-2-mehyl-N-[I-(3-{[(3-{[(4-methylphenyl)oxy]methyl}-1,2,4-oxadiazol-5-yl)methyl]oxy}phenyl)cthyl]pynmidin-4-amine;
6-(1,3-benzotlnazol-6-yl)-2-methyl-N-{I-[3-(3-thienyl)phenyl]ethyl}pyrimidin-4-amine;
6-(1,3-benzotlnazol-6-yl)-N-f1-(3-{[2-(II-1-iniidazol-I-yl)ethyrjoxy}phenyl)ethyl]-2-methylpyrimidin-4-amine;
2-{[3-(1-{[2-methyl-6-(3-methyl-I-benzoluran-5-yl)pyrimidin-4-yl]amino}ethyl)phenyl]o.\y}acetamide;
N-[I-(3-{[(1,3-dimelhyl-]l-I-pyrazol-5-yl)methyl]oxy}phenyl)ethyl]-2-methyl-6-(3-met!iyl-I-benzofuran-5-yl)pyrimidin-4-amine;
2-{[3-(1-{[6-(1,3-benzothiazol-6-yl)-2-methylpyrimidin-4-yl]amino}ethyl)phenylJoxy}acetamide;
2-melhyl-6-(3-melhyl-1-benzo(′ ran-5-yl)-N-(1-{3-[(2-moi holin-4-yl-2-oxoethyi)oxy]phenyl}ethyl)pyrimidin-4-amine;
N,N-dimethyl-2-{[3-(1-{[2-metliyl-6-(3 methyl-1-benzofuran-5-yl)pyrimidin-4-yl]amino}elhy])phenyl]oxy}acetamide;
6-(1,3-benzothiazol-6-yl)-2-methyl-N-[1-(3-{[(I-methyl-1H-pyrazol-3-yl)methyl]oxy}phenyl)ethyl]pyrimidin-4-amine;
6-(1>3-benzothiazol-6-yl)-N-(I-{3-[(2-fluoroethyl)oxy]phenyl}ethyl)-2-methylpyrimidin-4-amine;
6-(1,3-benzothiazol-6-yl)-2-methyl-(I-{3-[(2:2,2-tri{Iuoroethyl)oxy]phenyl}ethyl)pyrimidin-4-amine;
N-[I-(3-bromo-4-fluoiOphenyl)ethyl]-2-methyl-6-(3-methyl-1-benzofuran-5-yl)pyrimidin-4-amine;
5-[2-iluoro-5-(I-{[2-methyl-6-(3-methyl-1-benzofuran-5-yl)pyrimidin-4-yl]amino}ethyl)phenyl]pyrimidin-2-amine;
N—{(IR)-I-[4-iluoro-3-(I-methyl-1H-pyrazol-4-yl)phenyl]ethyl}-2-methyl-6-(3-methyl-1-benzofuran-5-yl)pyrimidin-4-amine;
N—{(IS)—I-[4-I:luoro-3-(I-melhyl-1H-pyrazol-4-yl)phenyl]ethyl}-2-methyl-6-(3-metliyl-I-benzoruran-5-yl)pyrimidin-4-ainine;
5-[3-(I-{[6-(1,3-benzothiazol-6-yl)-2-methylpyrimidin-4-yl]amino}ethyl)phenyl]pyridine-3-carboxamide;
6-(1,3-benzothiazol-6-yl)-2-methyl-N-[(IS)-1-{3-[2-(4-methylpiperazin-I-yl)pyrimidin-5-yl]phenyl}ethyl]pyrimidin-4-amine;
6-(1,3-benzothiazol-6-yl)-2-methyl-N-(I-{3-[5-(trifluoiOmediyl)pyridin-3-yl]phenyl}ethyl)pyriniidin-4-amine;
2-methyl-6-(3-methyl-1-benzoiuran-5-yl)-N-[(1S)-1-{3-[2-(4-methylpiperazin-1-yl)pyrimidin-5-yl]phenyl}ethyl]pyrimidin-4-amine;
3-[(5-{3-[(IS)-1-{[2-methyl-6-(3-methyl-1-benzo{′uran-5-yl)pyrimidin-4-yl]amino}ethyl]phenyl}pyrimidin-2-yl)amino]propan-1-ol; methyl N-(5-{3-[(IS)-1-{[2-methyl-6-(3Miiethyl-I-benzofuran-5-yl)pyrimidin-4-yl]amino}ethyl]pheny!}pyrimidin-2-yl)g]ycinate;
2-methyl-6-(3-methyl-I-benzoiuran-5-yl)-N-[(IS)-1-{3-[6-(4-methylpiperazin-I-yl)pyridin-3-yrlphenyl}ethyl]pyrimidin-4-amine;
2,2-dimethyl-3-[(5-{3-[(IS)-1-{[2-methyl-6-(3-methyl-I-benzoftiran-5-yl)pyrim 4-yl]amino}ethyl]phenyl}pyrimidin-2-yl)amino]pi pan-1-ol;
N-(5-{3-[(I S)—I-{[2-methyl-6-(3-methyl-I-benzofuran-5-yl)pyrimidin-4-yl]amino}ethy!]phenyl}pyrimiclin-2-yl)glycine;
3-[(5-{3-[(IS)-1-{[2-methyl-6-(3-methyl-I-benzoftiran-5-yi)pyrimidin-4-yl]annno}ethyl]phenyl}pyrimidin-2-yl)amino]piOpane-1,2-diol;
N-[(IS)—I-(5′-fluoro:3′-bipyndin-5-yl)ethyl]-2-methyl-6 3-methyl-1-benzofuran-5-yl)pyrimidin-4-amine;
N-tflS\hat{ }I-iG′-fluoro-S′-bipyridin-S-y ethyll\hat{ }-meUiyl-e-iS-methyl-I-benzoiuran-S-yl)pyrimidin-4-amine;
N—{(IS)—I-[5-(I-ethyl-IH-pyrazol-4-yl)pyridin-3-yl]ethyl}-2-methyl-6-(3-methyl-I-benzofuran-5-yl)pyrimidin-4-aniine;
6-chloro-5′-[(1S)-1-{[2-methy I-6-(3-methyl-1-benzo furan-5-yl)pyrimidin-4-yl]amino}ethylj-3,3′-bipyridin-5-amine;
5-{5-[(1S)-1-{[2-methy I-6-(3-methy 1-1-benzo furan-5-y I)pyrimidin-4-yl]amino}ethyl]pyridin-3-yl}pyrimidin-2-amine;
N-[ ]-(3-biOmo-5-fluoiOphenyl)ethyrj-2-methyl-6-(3-methyl-1-benzofuran-5-yl)pyrimidin-4-amine;
5-[3-fluoiO-5-(1-{[2-methyl-6-(3-methyl-1-benzo furan-5-yl)pyrimidin-4-yl]amino}ethyl)phenyl]pyrimidin-2-amine;
-{1-[3-iluoro-5-(I-methyl-IH-pyrazol-4-yl)phenyl]ethyl}-2-methyl-6-(3-methyl-I-benzofuran-5-yl)pyrimidin-4-amine;
1-(5-{5-[(1S)-1-{[2-methyl-6-(3-methyl-1-benzofuran-5-yl)pyrimidin-4-yl]amino}ethyl]pyHdin-3-yl}pyrimidin-2-yl)piperidin-4-ol
N-{1-[3-(5-amino-6-chloropyridin-3-yl)-5-nuorophenyl]etl′iyl}-2-methyl-6-(3-methyl-1-benzoiuran-5-yl)pyrimidin-4-amine;
N-[I-(3-bromo-4-methylphenyl)ethyl]-2-melhyl-6-(3-methyl-1-benzofuran-5-yl)pyrimidin-4-amine;
5-[2-methyl-5-(1-{[2-methyl-6-(3-methyl-1-benzofuran-5-yl)pyrimidin-4-yl]amino}ethyl)phenyl]pyrimidin-2-amine; 2-methyl-6-(3-methyl-1-benzofuran-5-yl)-N—{\hat{ }I-[4-methyl-3-(I-methyl-1H-pyrazol yl)phenyl]ethyl}pyrimidin-4-amine;
5-{3-[(IS)-1-{[2-methyl-6-(I-methyl-Il-I-indol-6-yl)pyrimidin-4-yl]amino}ethyl]phenyl}pyi′imidin-2-amine;
2-methyl-6-(3-methyl-1-benzofuran-5-yl)-N—{(I S)-1-[5-(I-methyl-IH-pyrazol-5-yl)pyridin-3-yl]ethyl}pyrimidin-4-amine;
2-methyl-6-(3-methyl-1-benzofuran-5-yl)-N—{(IS)-1-[5-(IH-pyrazol-4-yl)pyridin-yl]elhyl}pyrimidin-4-amine;
(2S)-3-[(5-{3-[(IS)-1-{[2-methyl-6-(3-methyl-1-benzofuran-5-yl)pyrimidin-4-yl]amino}ethyl]phenyl}pyrimidin-2-yl)amino]propane-1,2-diol
2-melhyl-N-{1-[3-(1-methyl-1H-pyrazol-4-yl)phenyl]ethyl}-6-[4-(trifluo!′ometliyl)phenyl]pyrimidin-4-amine;
6-(2,3-dihydiO-1,4-benzodioxin-6-yl)-2-methyl-N-{1-[3-(I-methyl-1H-pyrazol-4-yl)phenyl]ethyl}pyrimidin-4-amine;
2-methyl-6-(3-methyl-1-benzofuran-5-yl)-N—{(1S)-1-[3-(1H-pyrazol-1-yl)phenyl]ethyl}pyi-imidin-4-amine;
2-nu′thyl-6-(3-methyl-1-benzofuran-5-yl)-N—{(IS)—I-[3-(2H-1,2,3-triazol-2-yl)phenyl]ethyl}pyrimidin-4-amine;
6-(1-benzothien-5-yl)-2-methyl-N-{I-3-(1-methyl-1H-pyrazol-4-yl)phenyl]ethyl}pyrimidin-4-amine;
N′-{5-[3-(1-{[6-(13-benzolhiazol-6-yl)-2-methylpyrimidin-4-yl]amino}ethyl)phenyl]pyrimidin-2-yl}-IS,N-dimethylethane-I,2-diamine;
2-methyl-6-(3-methyl-1-benzofuran-5-yl)-N—{(IS)—I-[3-(IH-tetrazol-I-yl)phenyl]ethyl}pyrimidin-4-amine;
2-methyl-6-(3-methyl-1-benzofuran-5-yl)-N—{(IR)-I-[3-(IH-tetrazol-I-yl)phenyl]ethyl}pyrimidin-4-amine;
N—{(IS)-1-[3-(2-{4-[2-(dimethylamino)ethyl]piperazin-1-yl}pyrimidin-5-yl)phenyl]ethyl}-2-methyl-6-(3-methyl-1-benzofuran-5-yl)pyrimidin-4-amine;
2-[4-(5-{3-[(IS)—I-{[2-methyl-6-(3-methyl-I-benzofuran-5-yl)pyrimidin-4-yl]a:nino}ethyl]phenyl}pyrimidin-2-yr)piperazin-I-yl]ethanol
2-methyl-6-(3-methyl-1-benzof uran-5-yl)-N-{(1S)-1-[3-(2-{4-[(1-methyl-1H-imidazol-2-yl)methyl]piperazin-I-yl}pyrimidin-5-yl)phenyl]ethyl}pyiimidin-4-ami
2-melhyl-6-(3-methyl-I-benzofiiran-5-yl)-N—{CIS)—I-[3-(2-{4-[2-(niethyloxy)ethyl]piperazin-I-yl}pyrimidin-5-yl)phenyl]ethyl}pyrimidin-4-amine; 2-({2-[4-(5-{3-[(IS)—I-{[2-methyl-6-(3 methyl-1-benzoairan-5-yl)pyrimidin-4-yl]amino}ethyl]phenyl}pyrimidin-2-yl)piperazin-I-yl]ethyl}oxy)ethanol
2-niethyl-6-(3 methyl-I-benzoiuran-5-yl)-N-[(IS)—I-(3-{2-[4-(2-morpholin-4-ylethyl)piperazin-I-y!]pyrimidii>5-yl}phenyl)ethyl]pynmidin-4-amine;
N—I(IS)—I-(3-bromopheiiyl)ethyl]-2-methyl-6-(3-mediyl-I-benzothien-5-yl)pyrimidin-4-amine;
2-methy I-6-(3-methyl-1-benzothien-5-yl)-N—{(1S)-1-[3-(1-methyl-1H-pyrazol-4-yl)phenyl]ethyl}pyrimidin-4-amine;
N—{(IS)—I-[3-(5-aminopyridin-3-yl)phenyl]ethyl}-2-methyl-6-(3-methyl-I-benzolhien-5-yl)pyrimidin-4-amine;
N—{(IS)-[3-(6-fluoropyridin-3-y!)phenyrjetliyl}-2-methyl-6-(3-methyl-1-benzothien-5-yl)pyrimidin-4-amine;
N—{(IS)—I-[3-(5-fluoropyridin-3-yl)phenyl]ethyl}-2-methyl-6-(3-methyl-1-benzothien-5-yl)pyrimidin-4-amine;
5-{3-[(I S)—I-{[6-(3-chloro-1-benzofuran-5-yl)-2-methylpyrimidin-4-yl]amino}ethyl]phenyl}pyrimidin-2-amine;
ethyl 5-[3-(1-{[6-(1,3-benzothiazol-6-yl)-2-methy lpyrimidin-4-yl]amino}ethyl)phenyl]pyridme-3-carboxylate;
6-(1,3-benzothiazol-6-yl)-N-(I-{3-[6-(dimethylamino)pyridin-3-yl]phenyl}ethyl)-2-methylpyrimidin-4-amine;
N-[(1S)-1-(3-bromophenyl)ethyl]-6-(3-ethyl-1-benzofuran-5-yl)-2-methylpyrimidin-4-amine;
5-{3-[(IS)-{[6-(I,3-benzothiazol-6-yl)-2-methylpyrimidin-4-yl]amino}ethyl]phenyl}pyrimidin-2-amine;
6-(1,3-benzothiaz;ol-6-yr)-N—{(IS)—I-[3-(5-nuoropyridin-3-yl)phenyl]ethyl}-2-methylpyrimidin-4-aniine:
6-(I;3-benzothiazol-6-yl)-2-methyl-N—{(IS)-1-[3-(6-methylpyridin-3-yl)phenyl]ethyl}pyrimidin-4-amine:
5-{3-[(IS)-{[6-(3-ethyl-1-benzofuran-5-yl)-2-methylpyrimidin-4-y.l]amiiio}ethyl]phenyl}pyrimidin-2-amine;
6-(1,3-benzothiazol-6-yl)-2-methyl-N—{(IS)—I-13-(5-methylpyndin-3-yl)phenyl]ethyl}pyrimidin-4-amine;
6-(133-benzothiazol-6-yl)-N—{(IS)—I-[3-(6-nuoi-5-methylpyridin-3-yl)phenyl]ethyl}-2-methylpyrimidin-4-aminc; 6-(1,3-benzothiazol-6-yl)-N—{(IS)—I-[3-(6-fluoropyridin-3-yl)phenyl]ethyl}-2-methylpyrimidin-4-amiiie;
N—{(IS)—I-[3-(5-fluoiOpyridin-3-yl)phenyl]elhyl}-2-methyl-6-(3-melhyl-1-benzofuran-5-yl)pyrimidin-4-amine;
6-(7-fluoro-I-benzofuran-5-yl)-2-methyl-N-{1-[3-(I-methyl-I H-pyrazol-4-yl)phenyl]ethyl}pyrimidin-4-amine;
N—{(IS)—I-[3-(2-chloropyrimidin-5-yl)p enyl]etliyl}-2-methyl-6-(3-methyl-I-benzofuran-5-yl)pyrimidin-4-amine;
6-(1,3-benzothiazol-6-yl)-N—{(IS)—I-[3-(2-chloropyrimidin-5-yl)phenj′I]ethyl}-2-methylpyrimidin-4-amine;
N-{(.IS)—I-[3-(6-Iluoropyridin-3-yl)plienyl]ethyl}-2-methyl-6-(3-methyl-I-benzofuran-5-yl)pyrimidin-4-amine;
5-{3-[(IS)-1-{[6-(1:3-beiizothiazol-6-yl)-2-metlwlpyrimidin-4-yl]amino}ethyl]p enyl}-N-methylpyrimidin-2-amine;
N-methyl-5-{3-[(IS)-1-{[2-metliyl-6-(3-melhyl-1-benzofuran-5-yl)pyrimidin-4-yl]amino}ethyl]phenyl}pyrimidin-2-amine:
5-{3-[(1S)-1-{[6-(7-fliioro-1-benzofuran-5-yl)-2-methylpyrimidin-4-yl]amino}ethyl]phenyl}pyrimidin-2-amine;
5-{3-[(IS)-1-{[6-(I,3-benzothiazol-6-yl)-2-methylpyrimidin-4-y]amino}ethyl]phenyl}-N,N-dimethylpyrimidin-2-amine;
N,N-dimethyl-5-{3-[(IS)-1-{[2-melhyl-6-(3-melhyl-1-benzofuran-5-yl)pyrimidin-4-yl]amino}ethyl]phenyl}pyrimidin-2-amine;
N-ethyl-5-{3-(IS)-1-{12-meUiyl-6-(3-metliyl-1-benzotiiran-5-yl)pyrimidin-4-yl]amino}ethyl]phenyl}pynmidin-2-amine;
5-{3-[(1S)-1-{[6-(1,3-benzothiazol-6-yl)-2-methy Ipyrimidin-4-yl]amino}ethyl]phenyl}-N-ethylpyrimidin-2-amine;
2-methyl-6-(3-methyl-I-benzoruran-5-yr)-N-{(] S)—I-[3-(2-morpholin-4-ylpyrimidin-5-yl)phenyl]ethyl}pyrimidin-4-amine;
2-methyl-6-(3-methyl-I-benzoiiiran-5-yl)-N—{(IS)—I-[3-(2-piperazin-1-ylpyrimidin-5-yl)phenyl]ethyl}pyrimidin-4-amine;
N—{(IS)—I-[3-(5-aminopyridin-3-yl)phenyl]etliyl}-2-methyl-6-(3-methyl-I-benzofuran-5-yl)pyrimidin-4-amine;
2-[(5-{3-[(IS)-1-{[2-methyl-6-(3-metliyl-1-benzol iran-5-yl)pyrimidin-4-yl]amino}ethyl]phenyl}pyrimidin-2-yl)amino]eilianol N,N-diethyl-5-{3-[(IS)-1-{[2-metliyl-6-(3-met yl-1-benzo-furan-5-yl)pyrimidin-4-yl]amino}ethyl]phenyl}pyrimidin-2-amine;
N—{(IS)—I-[3-(5-chloropyridin-3-yl)phenyl]ethyl}-2-melhyl-6-(3-methyl-I-benzofuran-5-yl)pyrimidin-4-amine;
6-(1:3-benzothiazol-6-yl)-N—{(IS)—I-[3-(5-chloropyridin-3-yl)phenyl]ethyl}-2-methylpyrimidin-4-amine;
5-{3-[(IS)-{[6-(L3-benzolhiazol-6-yl)-2-methylpyrimidin-4-yl]amino}ethyl]phenyl}-N,N-diethylpyrimidin-2-amine;
6-(1,3-benzothiazol-6-yl)-N-[(IS)-1-{3-[2-(4-ethyipiperazin-1-yl)pynmidin-5-yl]phenyl}ethyl]-2-methylpyrimidin-4-amine;
I-(5-{3-[(IS)-1-{[6-(L3-beiizothiazol-6-yl)-2-methylpyrimidin-4-yl]amino}ethyl]phenyl}pyrimidin-2-yl)piperidin-4-ol
N-[(1S)-1-{3-[2-(4-ethylpiperazin-1-yl)pyrimidin-5-yl]phenyl}ethyl]-2-methyl-6-(3-methyl-I-benzofuran-5-yl)pyrimidin-4-amine;
I-(5-{3-[(IS)-1-{[2Hiunhyl-6-(3-methyl-1-benzofuran-5-yl)pyrimidin-4-yl]amino}ethyl]phenyl}pyrimidin-2-yl)piperidin-4-ol;
1-(5-{3-[(I S)-1-{[2-methyl-6-(3-niethyl-I-benzofuran-5-yl)pyiirnidin-4-yl]amino}ethyl]phenyl}pynmidin-2-yl)pyn′olidin-3-ol;
N-(I-methylethyl)-5-{3-[(IS)-1-{[2-mel yl-6-(3-methyl-1-benzofuran-5-yl)pyrimidin-4-yl]amino}ethyl]phenyl}pyrimidin-2-amine;
[I-(5-{3-[(IS)-1-{[2-methyl-6-(3-methyl-1-benzofuran-5-yl)pyrimidin-4-yl]amino}ethyl]phenyl}pyrimidin-2-yl)piperidin-4-yl]methanol;
N-[(IS)-1-{3-[2-(4-fluoropiperidin-1-yl)pynmidin-5-yl]phenyl}ethy]]-2-rnethyl-6-(3-methyl-1-benzofuran-5-yl)pyrimidin-4-amine;
N-(fiiran-2-ylmelhyl)-5-{3-(IS)-1-{2-methyl-6-(3-methyl-1-benzofuran-5-yl)pyrimidin-4-yl]amino}ethyl]phenyl}pyrimidin-2-amiiie;
N iuran-3-ylmetliyl)-5-{3-[(IS)-1-{2-melhyl-6-(3-methyl-1-benzofuran-5-yl)pynmidin-4-yl]amino}ethyjphenyl}pynmidin-2-amine;
6-(7-nuoro-3-methyl-1-benzofuran-5-y I)-2-methy I-N-{1-[3-(1-methyl-1H-pyrazol-4-yl)phenyl]ethyl}pyrimidin-4-amine;
(5-{3-[(1S)-1-{[6-(L3-benzothiazol-6-yl)-2-methylpyrimidin-4-yl]amino}ethyl]phenyl}pyridin-2-yl)melhanol
2-methyl-6-(3-methyl-I-beirofuran-5-yl)-N—{(IS)—I-[3-(4-methyl-3,4-dihydro-2H-pyrido[3,2-b] [1,4]oxazin-7-yl)phenyl]ethyl} pyrimidin-4-aniine; (5-{3-[(IS)-1-{[6-(L3-benzothiazol-6-yl)-2-methylpyrimidin-4-yl]amino}ethyl]phenyl}pyridin-3-yl)methanol
N—{(IS)—I-[3-(5-amino-6-chloropyridin-3-yl)plienyl]ethyl}-2-methy]-6-(3-methy]-1-benzofuran-5-yl)pyrimidin-4-amine:
6-(7-fluoro-3-methyl-1-benzofuran-5-yl)-2-methyl-N-{(1S)-1-[3-(1-methyl-1H-pyrazol-4-yl)phenyl]ethyl}pyrimidin-4-amine;
6-(3,4-difluorophenyl)-2-methyl-N-{1-[3-(1-methyl-1H-pyiazol-4-yl)phenyl]ethyl}pyrimidin-4-amine;
(5-{3-[(1S)-1-{[2-methyl-6-(3-methyl-1-benzofuran-5-yl)pyrimidin-4-yl]amino}ethyl]phenyl}pyridin-3-yl)melhanol;
N-{(1S)-1-[3-(5-aminopyridin-3-yl)phenyl]ethyl}-6-(7-i:liioiO-3-methyl-1-benzofuran-5-yl)-2-melhylpyrimidin-4-amine;
6-(7-fluoro-3-niethyl-1-benzofuran-5-yl)-N—{(IS)-[3-(5-fluoiOpyridin-3-yl)phenyl]elhyl}-2-methylpyrimidin-4-amine;
6-(4-chloro-3,5-difluorophenyl)-2-methyl-N-{1-f 3-(1-methyl-1H-pyrazol-4-yl)phenyl]ethyl}pyrimidin-4-amine:
2-melhyl-N-{1-[3-(I-methyl-1H-pyrazol-4-yl)phenyl]ethyl}-6-(3,415-trifluoropheiiyl)pyrimidin-4-amine;
N—{(IS)—I-[3-(5-amino-6-chloropyridin-3-yl)phenyl]ethyl}-6-(7-fluoro-3-methyl-1-benzofuran-5-yl)-2-methylpyrimidin-4-amine;
5-{3-[(IS)—I-{[6-(3,4-dinuorophenyl)-2-methylpyrimidin-4-yl]amino}ethyl]phenyl}pyrimidin-2-amine;
5-{3-[(IS)-{[6-(4-chloro-3,5-dinuorophenyl)-2-methylpyrimidin-4-yl]amino}ethyl]phenyl}pyrimidin-2-amine;
1-(5-{3-[(1S)-1-{[2-methyl-6.-(3-methyl-1-benzofuran-5-yl)pyrimidin-4-yl]amino}ethyl]phenyl}pyridin-3-yl)ethanone;
6-(7-nuoro-3-methyl-1-benzofuran-5-yl)-2-methyl-N-{I-[5-(IH-pyrazol-4-yl)pyridin-3-yl]ethyl}pyrimidin-4-amine;
N-{I-[5-(I-ethyl-IH-pyrazol-4-yl)pyridin-3-yl]ethyl}-6-(7-fluoro-3-methyl-1-benzofuran-5-yl)-2-methylpyrimidin-4-amine;
6-(7-fluoro-3-methyl-I-benzofuran-5-yl)-N—{(IS)—I-[3-(6-fluoropyridin-3-yl)phenyl]ethyl}-2-methylpyrimidin-4-amine;
ethyl 5-{3-[(IS)—I-{[2-methyl-6-(3-methyl-1-benzoi:′uran-5-yl)pyrimidin-4-yl]amino}ethyl]phenyl}pyridine-3-carboxylate; 3-[(5-{3-[(IS)-1-{[6-(7-fluoro-3-niel yl-benzofuran-5-yl)-2-methylpyrimidin-4-yl]amino}ethyl]phenyl}pyrimidin-2-yl)amino]propane-1,2-diol
1-(5-{3-[(IS)—I-{[6-(7-fluoro-3-methyl-1-benzo uran-5-yl)-2-methylpyrimidin-4-yl]amino}ethyl]phenyl}pyrimidin-2-yl)piperidin-4-ol;
1-(5-{3-[(IS)-{[2-methyl-6-(3-melhyl-1-benzofuran-5-yl)pyrimidin-4-yl]amino}ethyl]phenyl}pyridin-3-yl)ethanol;
2-(5-{3-[(IS)-{[2-melhyl-6-(3-methyl-1-benzofuran-5-yl)pyrimidin-4-yl]amino}ethyl]phenyl}pyridin-3-yl)propan-2-ol:
6-(7-fluoro-3-methyl-1-benzofuran-5-yl)-2-melhy 1-N-{1-[5-(1-melhyl-1H-pyrazol-4-yl)pyridin-3-yl]ethyl} pyrimidin-4-amine;
5-[5-CI-{[6-(7-fluoro-3-methyl-1-benzofuran-5-yl)-2-methylpyrimidin-4-yl]amino}ethyl)pyridin-3-y]]pyrimidin-2-amine;
5-{3-[(IS)-{[6-(7-fluoro-3-methyi-benzofuran-5-yr)-2-methylpyrimidin-4-yl]amino}ethyl]phenyl}pyrimidin-2-aminc;
2-methyl-6-(3-methyl-1-benzofuran-5-yl)-N-[(I S)—I-(3-pyridin-3-ylphenyl)ethyl]pyrimidin-4-amine;
6-(1-benzothiazol-6-yl)-N—{I-[3-(2-fluoi pyridin-3-y])phenyl]ethyl}-2-methylpyrimidin-4-amine:
6-(1,3-benzothiazol-6-yl)-2-methyl-N-{1-[3-(2-methylpyridin-4-yl)phenyl]ethyl}pyrimidin-4-amine;
-[(IS)—I-{3-[6-(dimethylamino)pyridin-3-yl]phenyl}eth>4]-2-methyl-6-(3-methyl-I-benzofuran-5-yl)pyrimidin-4-amine;
6-(I,3-benzothiazol-6-yl)-2-methyl-N-{1-[3-(6-piperazin-I-ylpyridin-3-yl)phenyl]ethyl}pyrimidin-4-amine;
2-methyl-6-(3-methyl-I-benzofuran-5-yl)-N—{I-[5-(I-methyl-IH-pyrazol-4-yl)pyridin-3-yl]ethyl}py midin-4-amine;
2-methyl-6-(3-methyl-1-benzofuran-5-yl)-N-{(1S)-1-[3-(6-methylpyridin-3-yl)phenyl]ethyl}pyrimidin-4-amine;
2-methyl-6-(3-melhyl-1-benzofuran-5-yl)-N-{(1S)-1-[3-(6-piperazin-I-ylpyridin-3-yl)phenyl]ethyl}pyrimidin-4-amine;
2-methyl-6-(3-melhyl-I-benzofuran-5-yl)-N—{(IS)—I-[3-(5-methylpyridin-3-yl)phenyl]ethyl}pyrimidin-4-amine;
N—{(IS)—I-[3-(6-nuoro-5-methylpyridin-3-yl)phenyl]ethyl}-2-methyl-6-(3-methyl-I-benzofuran-5-yl)pyrimidin-4-amine: 6-(I,3-benzothiazol-6-yi)-N—{(IS)-]-[3-(2-Iuoropyridin-4-yl)phenyl]ethyl}-2-methylpyrimidin-4-amine;
5-[4-(]-{[2-methyl-6-(3-melhyl-I-benzofuran-5-yl)pyriniidin-4-yl]amino}ethyl)pyridin-2-yl]pynmidin-2-amine;
N-{(1S)-1-[3-(6-chloropyridin-3-yl)phenyl]ethyl}-2-methyl-6-(3-methyl-1-benzofuran-5-yl)pyrimidin-4-amine;
2-methyl-N-[(IS)—I-{3-[6-(methylamino)pyridin-3-yl]phenyl}ethyl]-6-(3-methyl-I-benzofuran-5-yl)pyrimidin-4-amine;
2-methyl-6-(3-methyl-I-benzofiu-an-5-yl)-N—{(IS)—I-[3-(6-morpholin-4-ylpyridin-3-yl)phenyl]etliyl}pyrimidin-4-amine;
2-[(5-{3-[(IS)—I-{[2-methyl-6-(3-methyl-I-benzofuran-5-yl)pyrimidin-4-yl]amino}ethyl]phenyl}pyridin-2-yl)amino]ethanol
6-(I,3-benzothiazo]-6-yl)-N—{(IS)—I-[3-(6-chloropyridin-3-yl)phenyl]ethyl}-2-methylpyrimidin-4-amine;
6-(I-beizof iran-5-yl)-2-rnethyl-N-{I-[3-(I-methyl-1H-pyrazol-4-yl)phenyl]ethy 1} pyrimidin-4-amine;
2-methyl-6-(3-methyl-I-benzoluraiv5-yl)-N—{(IR)-I-[2-(1-methyl-1H-pyrazol-4-yl)pyridin-4-yl]ethy]}pyrimidin-4-amine;
2-methyl-6-(3-methyl-I-benzofuran-5-yl)-N—{(I S)—I-[2-(I-methyl-1H-pyrazol-4-yl)pyridin-4-yl]ethyl}pyrimidin-4-amine;
N-[(1S)-1-{3-[6-(ethy Iamino)pyridin-3-y I]phenyl} ethyl]-2-methyl-6-(3-methyl-1-benzofuran-5-yl)pyrimidin-4-amine;
-[(IS)—I-{3-[6-(4-ethylpiperazin-I-yi)pyridiiv3-yl]phenyl}ethyl]-2-methyl-6-(3-methyl-I-benzofuran-5-yl)pyrimidin-4-amine:
6-(1,3-benzothiazol-6-yl)-N-[(IS)-1-{3-[6-(4-ethylpiperazin-1-yl)pyridin-3-yl]phenyl}ethyl]-2-methylpyrimidin-4-amine;
5-[5-(I-{[2-methyl-6-(3-methyl-1-benzofuran-5-yl)pyrimidin-4-yrj mino}ethyl)pyndin-3-yjpyrimidin-2-amine;
6-(I:3-benzothiazol-6-yl)-2-methyl-N-{(1S)-1-[3-(6-morpholin-4-ylpyridin-3-yl)phenyl]ethyl}pyrimidin-4-amine;
2-methyl-6-(3-methyl-1-benzofuran-5-yl)-N—{(1R)-1-[5-(1-methyl-1H-pyrazol-4-yl)pyridin-3-yl]ethyl}pyrimidin-4-amine;
I-(5-{3-[(IS)-1-{[2-methyl-6-(3-methyl-I-beiizofuran-5-yl)pyvimidin-4-yl]amino}ethylJphenyl}pyridin-2-yl)piperidin-4-ol 6-(K3-benzothiazol-6-yl)-N—{(IS)—I-[3-(6-chloiO-5-methylpyridin-3-yl)phenyl]ethyl}-2-methylpyrimidin-4-amine;
N—{(IS)—I-[3-(6-chloro-5-n thylpyridin-3-yl)phenyl]ethyl}-2-methyl-6-(3-methyl-1-benzofiiran-5-yl)pynmidin-4-amtne;
N-[I-(5-bromopyridin-3-yl)ethyl]-2-melhyl-6-(3-methyl-1-benzofuran-5-yl)pyrimidin-4-amine;
5′-(I-{[2-melhyl-6-(3Miiethyl-1-benzoiurai 5-yl)pyrimidin-4-yrjamino}ethyl)-3,3′-bipyridin-5-amine;
6-(4-chloiO-3-nuorophenyl)-2-methyl-N-{-[3-(I-methyl-1H-pyrazol-4-yl)phenyl]ethyl}pyrimidin-4-amine;
6-(3-iluorophenyl)-2-methyl-N-{(1S)-1-[3-(I-methyl-1H-pyrazol-4-yl)phenyl]ethyl}pyrimidin-4-amine;
5-{3-[(IS)-1-{[6-(4-chlorophenyl)-2-methylpyrimidin-4-yl]amino}ethy!]phenyl}pyrimidin-2-amine;
5-{3-[(IS)-1-{[6-(4-chloro-3-fluoiOphenyl)-2-methylpyrimidin-4-yl]amino}ethyl]phenyl}pyrimidin-2-amine;
N—{(IS)—I-[3-(5-aminopyridin-3-yl)phenyl]ethyl}-6-(4-chloro-3-tluorophenyl)-2-methylpyrimidin-4-amine;
5-[5-(1-{[6-(4-chloro-3-fluorophenyl)-2-mclhylpyrimidin-4-yl]amino}ethyl)pyridin-3-yl]pyrimidin-2-amine;
6-(4-chloro-3-nuorophenyl)-2-methyl-N-{I-[5-(I-methyl-1H-pyrazol-4-yl)pyridin-3-yl]ethyl}pyrimidin-4-amine;
6-(4-chloro-3-fluorophenyl)-N-{1-[5-(1-ethyl-1H-pyrazol-4-yl)pyridin-3-yl]ethyl}-2-methylpyrimidin-4-amine;
6-(4-chloro-3-Iluorophenyl)-2-methyl-N-{(1S)-1-[3-(6-piperazin-1-ylpyridin-3-yl)phenyl]ethyl}pyrimidin-4-arnine;
6-(4-chloiO-3-duorophenyl)-2-methyl-N-{(1S)-1-[3-(1-methyl-1H-pyiazol-4-yl)phenyl]ethyl}pyrimidin-4-amine;
6-(3-nuorophenyl)-2-methyl-{(1R)-1-[3-(1-methyl-1H-pyrazol-4-yl)phenyl]ethyl}pyrimidin-4-amine;
3-f(5-{3-[(IS)—I-{[6-(4-chloi-3-lliiorophenyl)-2-methylpyrimidin-4-yl]amino}ethyl]phenyl}pyrimidin-2-yl)amino]propane-1,2-diol I-(5-{3-[(IS)-{[6-(4-chloro-3-lluorophenyl)-2-methylpyrimidin-4-yl]amino}ethyl]phenyl}pynmidin-2-yl)piperidin-4-ol-{(IS)-[3-(4-chlorojDyridin-3-yl)phenyl]ethyl}-2-niethyl-6-(3-methyl-I-benzofuran-5-yl)pyrimidin-4-amine;
6-(4-chloro-3-fIuorophenyl)-N—{(IS)—I-[3-(I-ethy!-1H-pyrazol-4-yl)phenyl]ethyl}-2-methylpyrimidin-4-amine;
5-{3-[(IS)—I-{[2-methyl-6-(3 nelIiyl-I-benzofiiran-5-yl)pynrnidin-4-yl]amino}ethyl]phenyl}pyridine-3-carboxamide;
3-[2-methyl-6-({I-[3-(I-methyl-1H-pyrazol-4-yl)pheiiyr]ethyl}amino)pyrimidin-4-yl]phenol
6-(2,3-dihydro-I-benzoiuran-5-yl)-2-methyl-{1-[3-(I-methyl-1H-pyrazol-4-yl)phenyl]ethyl}pyrimidin-4-amine;
6-[2-fluoro-3-(methyloxy)phenyl]-2-methyl-N-{1-[3-(1-methyl-1H-pyrazol-4-yl)phenyl]ethyl}pyrimidin-4-amine;
6-(I,3-benzothiazol-6-yl)-2-methyl-N-(I-{3-[5-(methyloxy)pyridin-3-yl]phenyl}ethyl)pyrimidin-4-amine;
6-(I,3-benzothiazol-6-yl)-N—{I-[3-(2-fluoropyi′imidin-5-yl)phenyl]ethyl}-2-methylpyrimidin-4-amine;
5-[3-(I-{[6-(1,3-benzothiazol-6-yl)-2-methylpyrimidin-4-yl]amino}ethyl)phenyl]pyrimidine-2-carbonitrile;
6-(I,3-benzothiazol-6-yl)-N—{I-[3-(6-nuoro-2-methylpyndin-3-yl)phenyl]ethyl}-2-methylpyrimidin-4-amine;
5-[3-(I-{[6-(1,3-benzothiazol-6-yl)-2-methylpynmidin-4-yl]amino}ethyl)phenyl]pyridine-3-carbonitiile;
6-(I,3-benzothiazol-6-yl)-N—{I-[3-(4-chloropyridin-3-yl)phenyl]ethyl}-2-methylpyrimidin-4-amine;
6-(I,3-benzothiazol-6-yl)-N—{I-[3-(2,6-dimethylpyridin-3-yl)phenyl]ethyl}-2-methylpyrimidin-4-amine;
4-[3-(I-{[6-(1,3-benzothiazol-6-yl)-2-methylpyrimidin-4-yl]amino}ethyl)phenyl]pyrimidin-2-amine:
6-(I,3-benzothiazol-6-yl)-2-methyl-{(I S)-1-[3-(2-piperidin-I-ylpyrimidin-5-yl)phenyl]ethyl}pyrimidin-4-amine;
N′-(5-{3-[(IS)—I-{[6-(I,3-benzothiazol-6-yl)-2-methylpyrimidin-4-yl]amino}ethyl]phenyl}pyrimidin-2-yl)-N,N-dimethylpropane-I,3-diamine;
N-[(IS)—I-{3-[2-(4-acetylpiperazin-I-yl)pynniidin-5-yl]phenyl}ethyl]-2-methyl-6-(3-methyl-I-benzofuran-5-yl)pyrimidin-4-amine; 5-{3-[(1S)-1-{[2-methyl-6-(3-metliyl-1-benzofuran-5-yl)pyrimidin-4-yl]amino}ethyl]phenyl}-N-(telrahydrofuran-2-ylmethyl)pyrimidin-2-aminc;
6-(3-amino-4-chlorophenyl)-2-methyl-N-{1-[3-(I-methyl-1H-pyrazol-4-yl)phenyl]ethyl}pynmidin-4-amine;
6-(4-chloio-3-methylphenyl)-2-methyl-N-{I-[3-(1-methyl-1H-pyrazol-4-yl)phenyl]ethyl}pyrimidin-4-amine;
2-i]uoro-4-[2-methyl-6-({I-[3-(I-methyl-IH-pyrazol-4-yl)phenyl]ethyl}amino)pyrimidin-4-yl]beiizamide;
6-(I,3-benzothiazol-5-yl)-2-methyl-N-{1-[3-(I-methyl-1H-pyrazol-4-yl)phenyl jethyl} pyrimidin-4-amine;
N-[(IS)—I-{3-[2-(4-acetylpiperazin-1-yl)pyrimidin-5-yl]phenyl}ethyl]-2-methyl-6-(3-methyl-1-benzothien-5-yl)pyrimidin-4-amine;
N—{(IS)—I-[3-(2-{4-[2-(dimethylamino)ethyl]piperazin-yl}pyrimidin-5-yl)phenyl jethyl}-2-methyl-6-(3-methyl-1-benzothien-5-yl)pyrimidin-4-amine;
5-{3-[(IS)—I-{[2-methyl-6-(3-methyl-1-benzofuran-5-yl)pyrimidin-4-yl]amino}ethyl]phenyl}pyridine-3-carbonitrile;
2-methyl-6-(3-methyl-1-benzofuran-5-yl)-N-[(IS)—I-(6′-methyl-3, 3′-bipyridin-5-yl)ethyl]pyrimidin-4-amine;
2-methyl-6-(3-methyl-I-benzoi′uran-5-yl)-N—{(]S)—I-[5-(I-melhyl-1H-pyrazol-4-yl)pyridin-3-yl]ethyl}pyrimidin-4-amine;
5-{3-[(IS)—I-{[2-methyl-6-(3-methyl-1-benzothien-5-yl)pyrimidin-4-yl]amino}ethyl]phenyl}pynmidin-2-amine;
N—{(IS)—I-[3-(5-aminopyridin-3-yl)phenyl]ethyl}-6-(1:3-benzothiazol-6-yl)-2-methylpyrimidin-4-amine;
5-{3-[(IS)-1-{[2-methyl-6-(3-methyl-I-benzoluran-5-yl)pyrimidin-4-yl]amino}ethyl]phenyl}pyrimidin-2-ol;
(S)-5-(3-(I-(6-(7-fliioiO-3-methylbeiizofuran-5-yl)-2-methylpyrimidin-4-amino)ethyl)phenyl)pyrimidin-2-amine;
(S)-2-methyl-N-(I-(5-(I-methyl-1H-pyrazol-4-yl)pyridin-3-yl)ethyl)-6-(3-methylbenzofuran-5-yl)pyrimidin-4-amine;
(S)—N—(I-(3-(5-aminopyridin-3-yl)phenyl)ethyl)-6-(4-chloiO-3-fluoiOphenyl)-2-methy I py ri m i d in-4-amine;
(S)-5-(3-(I-(2-methyl-6-(3-methylbenzofuran-5-yl)pyrimidin-4-ylamino)ethyl)phenyl)pynmidin-2-amine;
N-(I-(5-(1-ethyl-1 WO20161805379H-pyrazol-4-yl)pyridin-3-yl)elhyl)-6-(7-fluoiO-3-methylbenzofuran-5-yl)-2-methylpyrimidin-4-amine:
5-(3-fluoro-5-(I-(2-niethyl-6-(3-methylbenz iuran-5-yl)pyrimidin-4-ylamino)ethyl)phenyl)pyrimidin-2-amine;
(S)-5-(3-(I-(2-metliyl-6-(3-methylbenzo[b]thiophen-5-yl)pyrimidin-4-ylamino)ethyl)phenyl)pyrimidin-2-amine; or
(S)—N-(I-(3-(5-aminopyridin-3-yl)phenyl)ethyl)-2-methyl-6-(3-methylbenzo[b]thiophen-5-yl)pyrimidin-4-amine.

43. A pharmaceutical composition comprising a compound of items 1-42 and a pharmaceutically acceptable carrier, excipient. or diluent.

Item E

PFKFB3 inhibitor for use in neuroprotection, wherein PFKFB3 is selected from those described in WO2016180537A1 incorporated here by reference.

PFKFB3 inhibitor for use in neuroprotection, wherein PFKFB3 inhibitor is selected from the following

1. Compound of formula (I)

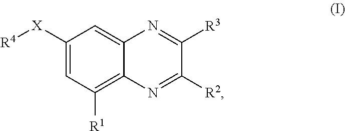

wherein X denotes N—R⁵ or O; R¹ denotes Ar^{X}, Ar^{X}—Ar^{Y}, Ar^{X}-Hetar^{Y} Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{A}-Hetar^{A} Hetar^{x}-Hetcyc^{Y}, Hetai\hat{ }-LA^{A}Ar^{A} Hetai\hat{ }-LA^{A}Heta^{A}, Hetai\hat{ }-LA^{Z}-Hetcyc^{Y}, Hetcyc^{x}, Hetcyc^{x}-Ar^{Y}, Hetcyc^{x}-Hetar^{Y}, Hetcyc^{x}- Hetcyc^{Y}, Hetcyc^{x}-LA^{Z}-Ar^{Y}, Hetcyc^{x}-LA^{z}-Hetar^{Y} Hetcyc^{x}-LA^{z}-Hetcyc^{Y}, CA^{X};

R² and R³ denote independently from each other H, —OH, —SH,

straight-chain or branched —C₁₋₆-alkyl, straight-chain or branched —C₂₋₆-alkenyl, straight-chain or branched —O—Ci-6-alkyl, straight-chain or branched —S-C-₆-alkyl, Hal, —CN, —NH₂, —NH(C-4-alkyl),—N(Ci-4-alkyl)₂ which d-4-alkyl substituents may be the same or different and may be straight-chain or branched;

R⁴ denotes Ar or Hetar, which Ar or Hetar bears in its ortho-position (relative to the attachment of R⁴ to X) one (1) substituent R^{W1} and may or may not bear further substituents;

denotes H, Ai Hetar, Hetcyc^{x}, LA^{X} CA^{X}; w

Ar denotes a mono-, bi- or tricyclic aromatic ring system with 5, 6, 7, 8, 9, 10, 11, 12, 13, 14 ring carbon atoms which ring system may bear—besides the ortho-substituent R^{W1}—no further substituent or one (1) further substituent R^{W2} or two (2) further substituents

R^{W2}, R^{W3}, that may be the same or different;

Ar* denotes a mono-, bi- or tricyclic aromatic ring system with 5, 6, 7,

8, 9, 10, 11, 12, 13, 14 ring carbon atoms which ring system may be unsubstituted or mono-, di- or trisubstituted with

independently from each other RX, R^{X2}, R^{X3};

Ar^{Y} denotes a mono-, bi- or tricyclic aromatic ring system with 5, 6, 7,

8, 9, 10, 11, 2, 13, 14 ring carbon atoms which ring system may be unsubstituted or mono-, di- or trisubstituted with

independently from each other R^{Y1}, R^{Y2}, R^{Y3};

Hetar denotes a mono-, bi- or tricyclic aromatic ring system with

5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that ring system may bear—besides the ortho-substituent R^{W1}-no further substituent or one (1) further substituent R^{W2} or two (2) further substituents R^{W2}, R^{W3}, that may be the same or different;

Hetar* denotes a mono-, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or tri-substituted with independently from each other R^{X1}, R^{X2}, R^{X3};

Hetar^{Y} denotes a mono-, bi- or tricyclic aromatic ring system with 5, 6, 7, 8, 9, 10, 1, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or tri-substituted with independently from each other R^{Y}, R^{Y2}, R^{Y3};

Hetcyc^{x} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{X4}, R^{x5}, R^{X6};

Hetcyc^{Y} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, R^{Y6};

R¹ denotes Hal, LA^{X}, CA^{X}, Ai\hat{ }, A\hat{ }-Ar. Ai\hat{ }-Hetar\hat{ }Ar^{x}-Hetcyc^{Y}, Ar^{x}-LA^{z}-Ar^{Y}, At\hat{ }-LA\hat{ }Hetar\hat{ }Ar^{x}-LA^{z}-Hetcyc^{Y}, Hetar\hat{ }, Hetai\hat{ }-Ar^{Y}, Hetai\hat{ }-Hetar\hat{ }Hetar^{x}-Hetcyc^{Y}, Hetai\hat{ }-LA\hat{ }Ar\hat{ }Hetai\hat{ }-LA²-Hetar^{Y}, Hetar-LA^{z}-Hetcyc^{Y}, Hetcyc^{x}, Hetcyc^{x}-Ar^{Y}, Hetcyc^{x}-Hetar^{Y}, Hetcyc^{x}-Hetcyc^{Y}, Hetcyc^{x}-LA^{z}-Ar^{Y}, Hetcyc^{x}-LA^{z}-Hetar^{Y}, Hetcyc^{x}-LA^{z}-Hetcyc^{Y}, —CN, —NO₂, —SO₂NH₂, —SO₂NHR^{W4},—SO₂NR^{W4}R^{W5}, —NH—SO₂—R^{W6}, —NR^{W4}—SO₂—R^{W6}, S-R^{W6}, —S(=0)-R^{W6}, —SO₂—R^{W6}, —NH₂, —NHR^{W4}, —NR^{W4}R^{W5}, —OH, —O—R^{W6}, —CHO, —C(═O)—R^{W6}, —COOH, —C(═O)—O—R^{W6}, —C(═O)—NH₂, —C(═O)— NHR^{W4}, —C(═O)—NR^{W}R^{W5}, —NH—C(═O)—R^{W6}, —NR^{W4}—C(═O)—R^{W6}, —NH—(C₁₋₃-alkylene)-C(═O)-NH₂, —NH-(Ci-₃-alkylene)-C(═O)—NHR^{W4}, —NH—(C₁₋₃-alkylene)-C(═O)—NR^{W4}R^{W5}, or

R^{W1} and R⁵ form together a divalent alkylene chain with 1, 2, 3,

4, 5 chain carbon atoms wherein 2 adjacent CH₂ groups may together be replaced by a —CH═CH— moiety, which divalent alkylene chain may be straight-chain or branched and may be unsubstituted or mono- or di-substituted with independently from each other straight-chain or branched —Ci-6-alkyl or ═O (oxo);

R^{W2}, R^{W3} denote independently from each other H, Hal, LA^{X}, CA^{X}, Ar*, Ar\hat{ }-A\hat{ }, Ai\hat{ }-Heta\hat{ }, Ar^{x}-Hetcyc^{Y}, Ai\hat{ }-LA\hat{ }Ar⁷, Ar\hat{ }-LA²-Hetar^{Y}, Ai\hat{ }-LA\hat{ }Hetcyc\hat{ } Hetai\hat{ }, Hetai\hat{ }-Ar\hat{ }Hetai\hat{ }-Hetar\hat{ }Hetar^{x}-Hetcyc^{Y}, Heta\hat{ }-LA\hat{ }A\hat{ }, Hetai\hat{ }-LA\hat{ }Hetar\hat{ }Hetar\hat{ }-LA²-

Hetcyc^{Y}, Hetcyc^{x}, Hetcyc^{x}-Ar^{Y}, Hetcyc^{x}-Hetar^{Y}, Hetcyc^{x}-Hetcyc^{Y}, Hetcyc^{x}-LA^{z}-Ar^{Y}, Hetcyc^{x}-LA^{z}-Hetar^{Y}, Hetcyc^{x}-LA^{z}-Hetcyc^{Y}, —CN, —NO₂, —SO₂NH₂, —SO₂NHR^{W4}, —SO₂NR^{W4}R^{W5}, —NH—SO₂—R^{W6}, —NR^{W4}—SO₂—R^{W6}, —S—R^{W6}, —S(═O)—R^{W6}, —SO₂—R^{W6}, —NH₂, —NHR^{W4}, —NR^{W4}R^{W5}, —NH—C(═O)—R^{W6}, —NR^{W4}—C(═O)—R^{W6}, —OH, —O—R^{W6}, —CHO, —C(═O)—R^{W6}, —COOH, —C(═O)—O—R^{W6}, —C(═O)—NH₂) —C(═O)— NHR^{W4}, —C(═O)—NR^{W4}R^{W5}, —C(═O)—NH—NH₂, —C(═O)—NH—NHR^{W4}, —NH—(C₁₋₃-alkylene)-C(═O)—NH₂, —NH-(Ci-₃-alkylene)-C(═O)—NHR^{W4}, —NH-(Ci-₃-alkylene)-C(═O)—NR^{W4}R^{W5}, or

two of R^{W1}, R^{W2} and R^{W3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(═C(═)-C₁₋₄-alkyl),

—O-wherein that C-i-6-alkyl and Ci\hat{ }-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched —C -₆-alkyl or =0 (oxo);

R^{X1}, R^{X2}, R^{X3} denote independently from each other other H, Hal, LA^{X}, CA^{X}, —CN, —N0₂, —SF₅, —S0₂NH₂, —S0₂NHR^{X7}, —S0₂NR^{X7}R^{X8}, —NH—S0₂—R^{X9}, —NR^{X7}—S0₂—R^{X9}, —S—R^{X9}, —S(═O)—R^{X9}, —SO₂—R^{X9},—NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH-(Ci-3-alkylene)-C(═O)— NH₂, —NH—(C₁₋₃-alkylene)-C(═O)—NHR^{x7}, —NH—(C₁₋₃-alkylene)-C(═O)—NR^{X7}R^{X8} or

two of R^{X1}, R², R^{X3} form a divalent alkylene chain with 3, 4, 5 chain carbon atoms wherein 1 or 2 of non-adjacent CH₂ groups of the divalent alkylene chain may be replaced independently from

each other by —N(H)—, —N(C₁₋₆-alkyl)-, —N(═C(═)-C₁₋₄-alkyl),

—O-wherein that Ci-₆-alkyl and C- -alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched -d-6-alkyl or =0 (oxo);

R^{X4}, R^{X5}, R^{X6} denote independently from each other H, Hal, LA^{X}, CA^{X}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH-(Ci-3-alkylene)-C(═O)— NH₂, —NH-(Ci-3-alkylene)-C(═O)—NHR^{X7}, —NH—(C₁₋₃-alkylene)-C(═O)—NR^{X7}R^{X8}, oxo (═O);

R^{Y1}, R^{Y2}, R^{Y3} denote independently from each other H, Hal, LA^{Y}, CA^{Y}, —CN, —NO₂, —SF₅, —SO₂NH₂, —SO₂NHR^{Y7}, —SO₂NR^{Y7}R^{Y8}, —NH—SO₂—R^{Y9}, —NR^{Y}—SO₂—R^{Y9}, —S—R^{Y9}, —S(═O)—R^{Y9}, —SO₂—R^{Y9}, —NH₂, —NHR\hat{ }⁷, —NR^{Y7}R^{Y8}, —OH, —O—R^{Y9}, —CHO, —C(═O)—R^{Y9}, —COOH, —C(═O)—O—R^{Y9}, —C(═O)—NH₂>-C\hat{ }OJ-NHR\hat{ }⁷, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)— NH₂, —NH-(Ci-3-alkylene)-C(═O)—NHR^{Y7}, —NH-(Ci-3-alkylene)-C(═O)—NR^{Y7}R^{Y8} or

two of R^{Y}, R^{Y2}, R^{Y3} form a divalent alkylene chain with 3, 4, 5 chain carbon atoms wherein 1 or 2 non-adjacent CH₂ groups of the divalent alkylene chain may be replaced independently from each other by —N(H)-, —N(Ci-₆-alkyl)-, —N(—C(═O)—C₁₋₄-alkyl), —O— wherein that Ci-₆-alkyl and Ci\hat{ }-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched —Ci-₆-alkyl or ═O (oxo);

R^{Y4}, R^{Y5}, R^{Y6} denote independently from each other H, Hal, LA^{Y}, CA^{Y}, —CN, —NO2, —SF₅, —SO2NH₂, —S0₂NHR^{Y7}, —SOsNR\hat{ }R, —NH—S0₂—R^{Y9}, —NR^{Y}—SO₂—R^{Y9}, —S—R^{Y9}, —S(=0)-R^{Y9}, —S0₂—R^{Y9}, —NH₂, —NHR\hat{ }, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═)R^{Y9}, —NH—(C₁₋₄-alkylene)-C(═O)—,

NH₂, —NH-(Ci-₃-alkylene)-C(═O)—NHR^{Y7}, —NH—(C₁₋₃-alkylene)-C(═O)—NR^{Y7}R^{Y8}, oxo (═O);

LA^{X} denotes straight-chain or branched Ci-₆-alkyl which may be

unsubstituted or mono-, di- or trisubstituted with independently from each other Hal, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)— R^{X9}, —COOH, —C(═O)—O—R^{X9}, —C(═O)—NH₂, —C(═O)—NHR^{X7}, —C(═O)— NR^{X7}R^{X8}, —NH—C(═O)—R^{X9}, —NR^{X7}—C(═O)—R^{X9}, —NH-(Ci-₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O), wherein 1 or 2 non-adjacent CH₂ groups of the Ci-₆-alkyl radical may independently from each other be replaced by O, S, N(H) or N—R^{X7} and/or 1 or 2 non-adjacent CH groups of the d-6-alkyl radical may independently from each other be replaced by N;

LA^{Y} denotes straight-chain or branched Ci-₆-alkyl which may be

unsubstituted or mono-, di- or trisubstituted with independently from each other Hal, —CN, —NO₂, —SF₅, —SO₂NH₂,

—SO₂NHR^{Y7}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR\hat{ }R, —OH, —O—R^{Y9}, —CHO, —C(═O)—R^{Y9}, —COOH, —C(═O)—O—R^{Y9}, —C(═O)—NH₂, —C(═O)— NHR^{Y}, —C(═O)—NR^{Y7}R^{Y8}, —NH—C(═O)—R^{Y9}, —NR^{Y7}—C(═O)—R^{Y9}, —NH—(C₁₋₃-alky!ene)-C(═O)—NH₂, —NH—(C₁₋₃-alkylene)-C(═O)—NHR^{Y7},—NH-(Ci-₃-alkylene)-C(═O)—NR^{Y7}R^{Y8}, oxo (═O), wherein 1 or 2 non-adjacent CH₂ groups of the Ci-₆-alkyl radical may

independently from each other be replaced by O, S, N(H) or N-R^{Y} and/or 1 or 2 non-adjacent CH groups of the Ci-₆-alkyl radical may independently from each other be replaced by N;

LA^{z} denotes a divalent straight-chain or branched C₁₋₆-alkylene

radical which alkylene radical may be unsubstituted or mono-, di- or trisubstituted with independently from each other Hal, —CN, —NO₂) —SF₅, —SO₂NH₂, —SO₂NHR^{Z7}, —SO₂NR^{Z7}R^{Z8}, —NH—SO₂—R^{Z9}, —NR^{Z7}—SO₂—R^{Z9}, —S—R^{Z9}, —S(═O)—R^{Z9}, —SO₂—R^{Z9}, —NH₂, —NHR^{Z7}, —NR^{Z7}R^{Z8}, —OH, —O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—C(═O)— R^{Z9}, —NR^{Z7}—C(═O)—R²⁹, —NH-(Ci-₃-alkylene)-C(═O)-NH₂, —NH-(Ci-₃-alkylene)-C(═O)—NHR^{Z7}, —NH-(Ci-₃-alkylene)-C(═O)—NR^{Z7}R^{Z8}, oxo (═O), wherein 1 or 2 non-adjacent CH₂ groups of that divalent alkylene radical may be replaced independently from each other by O, S, —N(H) or N—R^{Z7} and/or 1 or 2 non-adjacent CH groups of that divalent alkylene radical may be replaced by N;

R^{W4}, R^{W5}, R^{W6} denote Al\hat{ }, Ai\hat{ }-A\hat{ }, Ai\hat{ }-Hetar\hat{ }Ar^{x}-Hetcyc^{Y}, Ai\hat{ }-LA²-Ar^{Y}, Ai\hat{ }- LA\hat{ }Hetar\hat{ }Ar^{x}-LA^{z}-Hetcyc^{Y},

Hetai\hat{ }, Hetai\hat{ }-Ar\hat{ }Hetai\hat{ }-Hetar^{Y}, Hetar^{x}-Hetcyc^{Y}, Hetar\hat{ }-LA\hat{ }Ar\hat{ }Hetai\hat{ }-LA\hat{ }Hetar\hat{ }

Hetai\hat{ }-LA^{z}-Hetcyc^{Y}, Hetcyc^{x}, Hetcyc^{x}-Ar^{Y}, Hetcyc^{x}-Hetar^{Y}, Hetcyc^{x}-Hetcyc^{Y}, Hetcyc^{x}-LA^{z}-Ar^{Y}, Hetcyc^{x}-LA^{z}-Hetar^{Y}, Hetcyc^{x}-LA^{z}-Hetcyc^{Y}, LA^{X}, LA^{z}-Ar^{Y}, LA^{z}-Hetar^{Y}, LA^{z}-Hetcyc^{Y} CA^{X} or

R^{W4} and R^{W5} form together with the nitrogen atom to which they are

attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C\hat{ }-alky!;

_{RX8J RX9J R}Y_{71 R}Y_{8) R}Y_{9>R}Z7 _{RZ8J R}Z9 DENOTE INDEPENDENTLY from each other straight-chain or branched d-6-alkyl, which may be unsubstituted or mono-, di- or trisubstituted with

independently from each other Hal, —CN, —NO₂, —SF₅, —SO₂NH₂,—SO₂NHR^{X7v}, —SO₂NR^{X7v}R^{X8v}, —NH—SO₂—R^{X9v}, —NR^{X7v}—SO₂—R^{X9v}, —S—R^{X9v}—S(═O)—R^{X9v}, —SO₂—R^{X9v}, —NH₂, —NHR^{X7v}, —NR^{X7v}R^{X8v}, —OH,—O-R^{X9v}, —CHO, —C(═O)—R^{X9v}, —COOH, —C(═O)—O—R^{X9v}, —C(═O)—NH₂, —C(═O)—NHR^{X7v}, —C(═O)—NR^{X7v}R^{X8v}, —NH—C(═O)—R^{X9v}, —NR^{X7v}, —C(═O)—R^{X9v}, —NH—(C₁₋₃-alkylene)-C(═O)—NH₂, —NH—(C₁₋₃-alkylene)-C(═O)—NHR^{X7v}, —NH—(C₁₋₃-alkylene)-C(═O)—NR^{X7v}R^{X8v}, oxo (═O), wherein 1 or 2 non-adjacent CH₂ groups of the Ci-₆-alkyl radical may independently from each other be replaced by O, S, N(H) or N—R^{X7v} and/or 1 or 2 non-adjacent CH groups of the Ci-₆-alkyl radical may independently from each other be replaced by N, or a saturated monocyclic carbocycle with 3, 4, 5, 6, 7 carbon atoms, which may be unsubstituted or mono- or disubstituted with independently from each other Hal, Ar*, Ai\hat{ }-A\hat{ }, Ar\hat{ }-Heta\hat{ }, Ar^{x}-Hetcyc^{Y}, Ai\hat{ }-LA\hat{ }A\hat{ }, Ar^{x}-LA^{z}-Hetar^{Y}, Ar^{x}-LA^{z}-Hetcyc^{Y}, Hetai\hat{ }, Hetai\hat{ }-Ar″, Hetai\hat{ }-Hetar\hat{ }Hetar^{x}-Hetcyc^{Y}, Hetai\hat{ }-LA²-Ar^{Y}, Hetar^{x}-LA^{z}-Hetar^{Y}, Hetar-LA^{z}-Hetcyc^{Y}, Hetcyc^{x}, Hetcyc^{x}-Ar^{Y}, Hetcyc^{x}-Hetar^{Y}, Hetcyc^{x}-Hetcyc^{Y}, Hetcyc^{x}-LA^{z}-Ar^{Y}, Hetcyc^{x}-LA^{z}-Hetar^{Y}, Hetcyc^{x}-LA^{z}-Hetcyc^{Y}, LA^{X}, LA^{z}-Ar^{Y}, LA^{z}-Hetar^{Y}, LA^{Z}-Hetcyc^{Y}, —CN, —NO₂, —SF₅, —SO₂NH₂, —SO₂NHR^{X7v},—SO₂NR^{X7v}R^{X8v}, —NH—SO₂—R^{X9v}, —NR^{X7v}—SO₂—R^{X9v}, —S—R^{X9v}, —S(═O)— R^{X9v}, —SO₂—R^{X9v}, —NH₂, —NHR^{X7v}, —NR^{X7v}R^{X8v}, —OH, —O—R^{X9v}, —CHO, —C(═O)—R^{X9v}, —COOH, —C(═O)—O—R^{X9v}, —C(═O)—NH₂, —C(═O)— NHR^{X7v}, —C(═O)—NR^{X7v}R^{X8v}, —NH—C(═O)—R^{X9v}, —NR^{X7v}—C(═O)—R^{X9v}, —NH-(Ci-₃-alkylene)-C(═O)—NH₂>—NH—(C₁₋₃-alkylene)-C(═O)— NHR^{X7v}, —NH-(Ci-₃-alkylene)-C(═O)—NR^{X7v}R^{X8v}, oxo (═O), with the proviso that if any of the substituents of that monocyclic carbocycle is Ar*. Ai\hat{ }-A\hat{ }, A\hat{ }-Heta\hat{ }, Ai\hat{ }-Hetcyc⁷, Ai\hat{ }-LA\hat{ }Ar\hat{ }Ar\hat{ }-LA\hat{ }Hetar\hat{ }Ar^{x}-LA^{z}-Hetcyc^{Y}, Hetai, Hetai\hat{ }-Ar\hat{ }Hetai\hat{ }- Hetar^{Y}, Hetar\hat{ }-Hetcyc\hat{ }Hetai\hat{ }-LA\hat{ }A\hat{ }, Hetar\hat{ }-LA\hat{ }Hetar\hat{ }Hetai\hat{ }-LA^{z}-Hetcyc^{Y}, Hetcyc^{x}, Hetcyc^{x}-Ar^{Y}, Hetcyc^{x}-Hetar^{Y}, Hetcyc^{x}-Hetcyc^{Y}, Hetcyc^{x}-LA^{z}-Ar^{Y}, Hetcyc^{x}-LA^{z}-Hetar^{Y}, Hetcyc^{x}-LA^{z}-Hetcyc^{Y}, LA^{X}, LA^{z}-Ar^{Y}, LA^{Z}-Hetar^{Y}, LA^{Z}-Hetcyc^{Y}, then any radical R^{X7}, R^{X8}, R^{X9}, R⁷, R^{Y8}, R^{Y9}, R^{Z7}, R^{Z8}, R^{Z9} of any substituent of Ai\hat{ }, Ar^{Y} Hetai\hat{ }, Hetar^{Y}, Hetcyc^{X}, Hetcyc^{Y}, LA^{X} and LA^{Z} may not denote a mono- or disubstituted monocyclic carbocycle, or a saturated monocyclic heterocycle with 3, 4, 5, 6, 7 ring atoms wherein 1 or 2 ring atom(s) is/are heteroatom(s) selected from N, O and/or S and the remaining ring atoms are carbon atoms, wherein that heterocycle may be unsubstituted or substituted with straight-chain or branched Ci-₆-alkyl, —C(═O)—Ci. 6-alkyl (straight-chain or branched) and/or oxo (═O), or a phenyl, —CH₂-phenyl, -naphthyl, —CH₂-naphthyl, heteroaromatic ring system or -CH2-heteroaromatic ring system with 5, 6, 7, 8, 9, 10,

11 ring atoms, wherein 1, 2, 3, 4, 5 of said ring atoms of said heteroatomic ring system is/are hetero atom(s) selected from N, O and/or S and the remaining are carbon atoms, wherein said phenyl, naphthyl or heteroaromatic ring system may be unsubstituted or mono-, di- or trisubstituted with independently from each other straight-chain or branched C\hat{ }-alky! or —O—C₆-alkyl, Hal or —C(═O)C₁₋₆-alkyl

(straight-chain or branched); or

each pair R^{X7} and R^{X8}; R\hat{ } and R^{YB}; R^{zr} and R^{Z8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched Ci-₆-alkyl;

R^{X7v}, R^{X8v}, R^{X9v} denotes independently from each other straight-chain or branched Ci-₆-alkyl, which may be unsubstituted or

mono-, di- or trisubstituted with Hal, or a unsubstituted saturated monocyclic carbocycle with 3, 4, 5, 6, 7 carbon atoms; or

R^{x7v} and R^{X8v} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched Ci-₆-alkyl;

CA^{X}, CA^{Y} denote independently from each other a saturated

monocyclic carbocycle with 3, 4, 5, 6, 7 carbon atoms which carbocycle may be unsubstituted or mono- or disubstituted with independently from each other R^{CA1}, R^{CA2};

R^{CA1}, R^{CA2} denote independently from each other H, Hal, Ai\hat{ }, Ai\hat{ }-A\hat{ },

Ai\hat{ }-Hetar′″, Ar^{x}-Hetcyc^{Y}, Ai\hat{ }-LA^{A}Ar. Ai\hat{ }-LA^{A}Hetar^{A} Ai\hat{ }-LA²-Hetcyc^{Y}, Hetar, Hetai\hat{ }—Ar^{A} Hetar^{A}-Hetar^{A}

Hetar^{x}-Hetcyc^{Y}, Hetar^{x}-LA^{z}-Ar^{Y}, Hetai\hat{ }-LA^{A}Hetar^{A} Hetai\hat{ }-LA^{z}-Hetcyc^{Y}, Hetcyc^{x}, Hetcyc^{x}-Ar^{Y}, Hetcyc^{x}-Hetar^{Y}, Hetcyc^{x}-Hetcyc^{Y}, Hetcyc^{x}-LA^{z}-Ar^{Y}, Hetcyc^{x}-LA^{z}-Hetar^{Y}, Hetcyc^{x}-LA^{z}-Hetcyc^{Y}, LA^{X}, LA^{Z}-Ar^{Y}, LA^{z}-Hetar^{Y}, LA^{z}-Hetcyc^{Y}, —CN, —NO₂, —SF₅, —SO₂NH₂,—SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9},—CHO, —C(═O)—R^{X9}, —COOH, —C(═O)—O—R^{X9}, —C(═O)—NH₂, —C(═O)— NHR^{X7}, —C(═O)—NR^{X7}R^{X8}, —NH—C(═O)—R^{X9}, —NR^{X7}—C(═O)—R^{x9}, —NH—(C₁₋₃-alkylene)-C(═O)—NH₂, —NH—(C₁₋₃-alkylene)-C(═O)—NHR^{X7}, —

NH-(Ci-₃-alkylene)-C(═O)—NR^{X7}R^{X8}, oxo (═O), with the proviso that if R^{CA1} or R^{CA2} denotes Ai\hat{ }, Ai\hat{ }-Ar\hat{ }Ai\hat{ }-Hetar\hat{ }Ar^{x}-Hetcyc^{Y}, Ar^{x}-LA^{z}-Ar^{Y}, Ai\hat{ }-LA\hat{ }Hetar\hat{ }Ar^{x}-LA^{z}-Hetcyc^{Y}, Hetai\hat{ }, Hetar-Ar^{Y}, Hetai\hat{ }-Hetar⁷, Hetar^{x}-Hetcyc^{Y}, Hetar\hat{ }-LA\hat{ }Ar\hat{ }Hetar\hat{ }-LA²-Hetar^{Y}, Hetai\hat{ }-LA^{z}-Hetcyc^{Y}, Hetcyc^{x}, Hetcyc^{x}-Ar^{Y}, Hetcyc^{x}- Hetar^{Y}, Hetcyc^{x}-Hetcyc^{Y}, Hetcyc^{x}-LA^{z}-Ar^{Y}, Hetcyc^{x}-LA^{z}-Hetar^{Y}, Hetcyc^{x}-LA^{z}-Hetcyc^{Y}, LA^{z}-Ar^{Y}, LA^{z}-Hetar^{Y}, LA^{z}-Hetcyc^{Y}, then Ar, Ar^{Y}, Hetar\hat{ }, Hetar^{Y}, Hetcyc^{x}, Hetcyc^{Y} may not be substituted with CA^{X} or CA^{Y};

Hal denotes F, Cl, Br, I; or derivatives, N-oxides, prodrugs, solvates, tautomers or stereoisomers thereof as well as the physiologically acceptable salts of each of the foregoing, including mixtures thereof in all ratios.

2. Compound according to item 1, or derivatives, N-oxides, prodrugs, solvates, tautomers or stereoisomers thereof as well as the physiologically acceptable salts of each of the foregoing, including mixtures thereof in all ratios, wherein X denotes N—R⁵ or O;

R denotes Ai\hat{ }, Hetar*, Ar*-Ar^{Y}, Ai\hat{ }-Hetar\hat{ }

R² and R³ both denote H;

R⁴ denotes Ar^{w} or Hetar^{w}, which Ar^{w} or Hetar^{w} has in its ortho-position (relative to the attachment of R⁴ to X) one (1) substituent R^{W1} and may or may not bear further substituents;

R⁵ denotes H or LA^{X};

Ar^{w} denotes a monocyclic aromatic ring system with 6 ring carbon atoms which ring system may bear—besides the ortho-substituent R^{W1}-no further substituent or one (1) further substituent R^{W2}, wherein R^{W1} and R^{W2} may be the same or different;

Ar\hat{ } denotes a monocyclic aromatic ring system with 6 ring carbon atoms which ring system may be unsubstituted or mono- or di-substituted with independently from each other R^{X1}, R^{X2};

Ar^{Y} denotes a monocyclic aromatic ring system with 6 ring carbon atoms which ring system may be unsubstituted or mono- or di-substituted with independently from each other R^{Y}, R^{Y2};

Hetar^{w} denotes a monocyclic aromatic ring system with 5 or 6 ring atoms wherein 1, 2 or 3 of said ring atoms is/are nitrogen atom(s) and the remaining are carbon atoms, wherein that ring system may bear—besides the ortho-substituent R^{W1}—no further substituent or one (1) further substituent R^{W2} wherein R^{W1} and R^{W2} may be the same or different;

Hetar* denotes a mono- or bi-cyclic aromatic ring system with 5, 6, 9, 10 ring atoms wherein 1, 2, 3 or 4 of said ring atoms is/are a hetero atom(s) selected from N, O and/or S and the remaining are carbon atoms, wherein that aromatic ring system may be unsubstituted or mono- or di-substituted with independently from each other R^{X1}, R^{A};

Hetar^{Y} denotes a monocyclic aromatic ring system with 5 or 6 ring atoms wherein 1, 2 or 3 of said ring atoms is/are a nitrogen atom(s) and the remaining are carbon atoms, wherein that aromatic ring system may be unsubstituted or mono-substituted with R^{Y1};

Hetcyc^{x} denotes a saturated mono-cyclic heterocycle with 4, 5, 6, 7, ring atoms wherein 1 or 2 ring atom(s) is/are heteroatom(s) selected from N, O and/or S and the remaining ring atoms are carbon atoms, wherein that heterocycle may be unsubstituted or mono-, disubstituted or trisubstituted with R^{X4}, R^{X5}, R^{x6};

Hetcyc^{Y} denotes a saturated monocyclic heterocycle with 4, 5, 6, 7 ring atoms wherein 1 or 2 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, R^{Y6};

R^{W1} denotes LA^{X}, Hetai, Hetcyc^{x}, Hal, —CN, —OH, —O—R^{W6}, —SO₂NH₂, —SO₂NHR^{W4}, —SO₂NR^{W4}R^{W5}, —NH—SO₂—R^{W6}, —NR^{W4}—SO₂—R^{W6}, —SO₂—R^{W6}, —NH₂, —NHR^{W4}, —NR^{W4}R^{W5}, —C(═O)—OH, —C(═O)—O—R^{W6}, —C(═O)—NH₂, —C(═O)—NHR^{W4}, —C(═O)—NR^{W4}R^{W5}, —NH—C(═O)—R^{W6}, —NR^{W4}—C(═O)—R^{W6};

or R⁵ and R,′W1 form together a divalent alkylene chain with 1, 2, 3 chain carbon atoms;

R^{W2} denotes H, Hetar*. Hetcyc^{x}, Hal, LA^{X}, —CN, —OH, —O—R^{W6}, —NO₂, —NH₂, —NHR^{W4}, —NR^{W4}R^{W5}, —COOH, —C(═O)—O—R^{W6}, —C(═O)—NH₂, —C(═O)—NHR^{W4}, —C(═O)—NR^{W4}R^{W5}, —C(═O)—NH—NH₂, —NH—C(═O)—R^{W6}, —NR^{W4}—C(═)—R^{W6};

or R^{W1} and R,W2 form together a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), O-wherein that Ci_6-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched —Ci-₆-alkyl or ═O (oxo);

R^{X1}, R^{X2} denote independently from each other H, LA^{X}, —NH₂,

—NHR^{X7}, —NR^{X7}R^{X8}, Hal, —OH, —OR^{X9}, —SR^{X9}, —SF₅, —C(═O)—NH₂,—C(═O)—NHR^{X7}, —C(═O)—NR^{X7}R^{X8}, —NH-C(═O)—R^{X9},

or form a divalent alkylene chain with 3, 4, 5 chain carbon atoms wherein 1 or 2 non-adjacent CH₂ group(s) of the divalent alkylene chain may be replaced independently from each other by —O—, which divalent alkylene chain may be unsubstituted or mono- or di-substituted with independently from each other straight-chain or branched —Ci-₆-alkyl;

R^{Y}, R^{Y2} denote independently from each other LAY;

LA^{X} denotes straight-chain or branched Ci-₆-alkyl which may be unsubstituted or mono-, di- or trisubstituted with independently from each other Hal, —CN, —NH₂, —NHR^{X7}, —NR^{X7}R^{X8};

LA^{Y} denotes straight-chain or branched Ci_₆-alkyl;

LA^{Z} denotes an divalent straight-chain or branched

C-i-6-alkylene radical;

R^{X4}, R^{X5}, R^{X6} denote independently from each other H, Hal, LA^{X}, —C(═O)—R^{X9}, oxo (═O);

R^{Y4}, R^{Y5}, R^{Y6} denote independently from each other H, Hal, LA^{Y}, —C(═O)—R^{Y9}, oxo (═O);

R^{W4} denotes straight-chain or branched C₁₋₆-alkyl, saturated

monocyclic carbocycle with 3, 4, 5, 6, 7 carbon atoms, Ai\hat{ },

Hetai\hat{ }, Hetcyc^{x}, LA^{z}-Ar^{Y}, LA^{z}-Hetar^{Y} or LA^{z}-Hetcyc^{Y};

R^{W5}, R^{W6}, denote independently from each other straight-chain or branched Ci-6-alkyl, a saturated monocyclic carbocycle with 3, 4, 5, 6, 7 carbon atoms, Ar^{A}, Hetai\hat{ }, Hetcyc^{x}, LA^{z}-Ar^{Y}, -LA^{z}-Hetar^{Y} or LA^{z}-Hetcyc^{Y} or R^{W4} and R^{W5} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched Ci-6-alkyl;

R^{X7}, R^{x8}, R^{X9}, R^{Y9} denote independently from each other straight-chain or branched Ci-6-alkyl, which may be unsubstituted or mono-, di- or trisubstituted with Hal or monosubstituted with —NH₂, a saturated monocyclic carbocycle with 3, 4, 5, 6, 7 carbon atoms, or a saturated monocyclic heterocycle with 3, 4, 5, 6, 7 ring atoms wherein 1 or 2 ring atom(s) is/are heteroatom(s) selected from N, O and/or S and the remaining ring atoms are carbon atoms, wherein that heterocycle may be unsubstituted or substituted with straight-chain or branched Ci-s-alkyl, —C(═O)—C₁₋₆-alkyl (straight-chain or branched) and/or oxo (═O), or a phenyl, —CH₂-phenyl, -naphthyl, —CH₂-naphthyl, heteroaromatic ring system or —CH₂-heteroaromatic ring system with 5, 6, 7, 8, 9, 10, 11 ring atoms, wherein 1, 2, 3, 4, 5 of said ring atoms of said heteroatomic ring system is/are hetero atom(s) selected from N, O and/or S and the remaining are carbon atoms, wherein said phenyl, naphthyl or heteroaromatic ring system may be un substituted or mono-, di- or trisubstituted with independently from each other straight-chain or branched Ci-6-alkyl or —O—Ci-6-alkyl, Hal or —C(═O)—Ci-6-alkyl (straight-chain or branched)

and R^{X8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched Ci-6-alkyl;

denotes F, Cl, Br, I.

3. Compound according to any one of items 1 or 2, or derivatives, N-oxides, prodrugs, solvates, tautomers or stereoisomers thereof as well as the physiologically acceptable salts of each of the foregoing, including mixtures thereof in all ratios,

wherein X denotes N—R⁵ or O;

R¹ denotes Ar*¹ or Hetar*¹; R⁵ denotes H;

Ar*¹ denotes phenyl which may be unsubstituted or mono-substituted with R^{X1}a or di-substituted with independently from each other pX1a 2a.

Hetar*¹ denotes a bicyclic aromatic ring system with 9 ring atoms wherein (i) 1 of said ring atoms is a nitrogen atom or an oxygen atom or a sulfur atom and the remaining are carbon atoms; or (ii) 1 of said ring atoms is a nitrogen atom and 1 further of said ring atoms is an oxygen atom or a sulfur atom, wherein that further hetero atom may be adjacent or not adjacent to the nitrogen atom, and the remaining are carbon atoms; or (iii) 2 of said ring atoms are nitrogen atoms and the remaining are carbon atoms; or (iv) 2 of said ring atoms are nitrogen atoms and another of said ring atoms is an oxygen atom or a sulfur atom and the remaining are carbon atoms; or (v) 3 of said ring atoms are nitrogen atoms and the remaining are carbon atoms; wherein that aromatic ring system may be unsubstituted or mono-substituted with R^{x1b} or di-substituted with independently from each other R^{x1b}, R^{X2b};

^{a}, R\hat{ }³ denote independently from each other straight-chain or branched C-i-₆-alkyl, which Ci-₆-alkyl may be unsubstituted or mono-, di- or trisubstituted with F and/or Cl, straight-chain or branched —O—Ci-6-alkyl, which -0-Ci-₆-alkyl may be

unsubstituted or mono-, di- or trisubstituted with F and/or Cl, —OH, —SR^{X9}, —SF₅, F, Cl, Br, —NH₂, —NHR^{X7}, —NR^{X7}R^{X8}, —C(═O)— NH₂, —C(═O)—NHR^{X7}, —C(═O)—NR^{X7}R^{X8} or form together a —CH₂—CH₂—0-,a —0-CH₂—CH₂-0- or a —OCH₂—C(CH₃)₂— chain;

^{b}, R² denote independently from each other straight-chain or branched Ci-₆-alkyl, which C-₆-alkyl may be unsubstituted or mono-, di- or trisubstituted with F and/or Cl, Cl, Br, F, —OH, —NH₂) —NHR^{X7}, —NR^{X7}R^{X8}, —NH-C(═O)-methyl, —NH—C(═O)—CH₂—NH₂, —NH—C(═O)-pyrrolidin-2-yl;

R^{X8}_{J} R^{X9} denote independently from each other straight-chain or branched Ci-6-alkyl or a saturated monocyclic carbocycle with 3, 4, 5, 6, 7 carbon atoms

and R^{X8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched Ci-6-alkyl.

Compound according to any one of items 1 to 3, or derivatives, N-oxides, prodrugs, solvates, tautomers or stereoisomers thereof as well as the physiologically acceptable salts of each of the foregoing, including mixtures thereof in all ratios,

wherein R¹ denotes methylphenyl, 3-methylphenyl, ethylphenyl, 3-ethylphenyl, 4-ethylphenyl, trifluoromethylphenyl, 4-(trifluoromethyl)phenyl, dimethylphenyl, 2,5-dimethylphenyl, diethylphenyl, 3,5-diethylphenyl, methoxyphenyl, 3-methoxyphenyl, 4-methoxyphenyl, trifluoromethoxyphenyl, 3-trifluoromethoxyphenyl, methylsulfanylphenyl, 3-methylsulfanylphenyl, pentafluorosulfanylphenyl, 4-pentafluoro-A⁶-sulfanylphenyl, methoxy-methylphenyl (methoxy-tolyl), 2-methoxy-5-methyl phenyl, 5-methoxy-2-methylphenyl,

fluorophenyl, 4-fluorophenyl, bromophenyl, 3-bromophenyl, 4-bromophenyl, bromo-fluorophenyl, 4-bromo-3-fluorophenyl, bromo-methylphenyl, 4-bromo-2-methylphenyl, chloro-methoxyphenyl, 2-chloro-5-methoxy-phenyl, aminophenyl, 3-aminophenyl, 4-aminophenyl, amino-methylphenyl, 2-amino-5-methylphenyl, 3-amino-4-methylphenyl, amino-fluoro-phenyl, 4-amino-3-fluorophenyl, hydroxy-methylphenyl, 2-hydroxy-5-methylphenyl, dihydrobenzofuran-5-yl, indolyl, 1H-indol-6-yl, N-methyl-indol-6-yl, 1-ethyl-1H-indol-6-yl (A7-ethyl-indol-6-yl), 1-n-propyl-indol-6-yl, A/-isopropyl-indol-6-yl, difluoromethyl-indol-6-yl, 2-(difluoromethyl)-1H-indol-6-yl, dimethylindolyl, dimethylindol-6-yl, 1,4-dimethyl-1H-indol-6-yl, 1,5-dimethyl-1H-indol-6-yl, fluoro-methylindolyl, fluoro-1-methylindol-6-yl, 4-fluoro-1-methylindol-6-yl, 5-fluoro-1-methylindol-6-yl, 7-fluoro-1-methyl-indol-6-yl, dimethylaminophenyl, 3-A/,A/-dimethylaminophenyl,

dimethylamino-methylphenyl, 2-dimethylamino-5-methylphenyl, benzothiazolyl, benzothiazol-6-yl, benzothiazol-5-yl,

dimethyldihydrobenzofuranyl, 3,3-dimethyl-2,3-dihydro-1-benzofuran-5-yl, methylbenzofuranyl, methyl-benzofuran-5-yl, 3-methyl-benzofuran-5-yl, benzothiophenyl, benzothiophen-5-yl, methylbenzothiophenyl, 3-methyl-1-benzothiophen-5-yl, trifluoromethyl-benzothiophenyl, 3-(trifluoromethyl)-1-benzothiophen-5-yl, aminobenzothiophenyl, 2-amino-1-benzothiophen-5-yl, 2-amino-1-benzothiophen-6-yl, 2-(acetylamino)-1-benzothiophen-5-yl, 2-(NH₂—CH₂—C(═O)NH-)-1-benzothiophen-5-yl, 2,3-dihydrobenzo[1,4]dioxin-6-yl, 1-methyl-1H-pyrrolo[2,3-b]pyrdin-6-yl, 1,2-benzothiazol-5-yl, 1,3-benzothiazol-5-yl, 1,3-benzothiazol-6-yl, 2-amino-1,3-benzothiazol-5-yl, 2-amino-1,3-benzothiazol-6-yl, 2-methylamino-1,3-benzothiazol-5-yl, 2-dimethylamino-1,3-benzothiazol-5-yl, 2-(acetylamino)-1,3-benzothiazol-5-yl, 2-(pyrrolidin-2-yl-C(=0)-NH—)-1,3-benzothiazol-5-yl, 2-(pyrrolidin-2-yl-C(=0)-NH—)-1,3-benzothiazol-6-yl, benzothiazololyl (hydroxybenzothiazolyl, dihydro-benzothiazolonyl), 1,3-benzothiazol-2-ol-5-yl (2-hydroxy-1,3-benzothiazol-5-yl, 2,3-dihydro-1,3-benzothiazol-2-on-5-yl), benzoxadiazolyl, 2,1,3-benzoxadiazol-5-yl, benzothiadiazolyl, 2,1,3-benzothiadiazol-5-yl, benzotriazolyl, 1,2,3-benzotriazol-5-yl.

Compound according to any one of items 1 to 4, or derivatives, N-oxides, prodrugs, solvates, tautomers or stereoisomers thereof as well as the physiologically acceptable salts of each of the foregoing, including mixtures thereof in all ratios,

wherein R⁴ denotes Ar^{W4} or Hetar^{W4};

Ar^{W4} denotes phenyl which is substituted with R^{w1a} in the ortho-position (relative to the attachment of Ar^{W4} to X) and may bear no further substituent or one further substituent R^{W2a};

Hetar^{W4} denotes a monocyclic aromatic ring system with 5 or 6 ring atoms wherein 1, 2 or 3 of said ring atoms is/are nitrogen atom(s) and the remaining are carbon atoms, wherein that ring system is substituted with R^{w1b} in the ortho-position (relative to the attachment of Hetar^{W4} to X) and may bear no further substituent or one further substituent R^{W2b};

R^{w1a}, R^{wb} denote independently from each other LA^{Xa}, Hetar*⁴,

Hetcyc^{X4}, Hal, —CN, —OH, —O—R^{W6a}, —SO₂NH₂, —SO₂NHR^{W4a}, —SO₂NR^{W4a}R^{W5a}, —SO₂—R^{W6a}, —NH₂,—NHR^{W4a}, —NR^{Wa}R^{W5a}—C(═O)— OH, C(═O)—O—R^{W6a}, —C(═O)—NH₂, —C(═O)—NHR^{W4a}, —C(═O)— NR^{W4a}R^{W5a};

R^{W2a}, R^{W2b} denote independently from each other H, Hal, I_A^{x}a, —CN, —NO₂, —NH₂, —NHR^{W4b}, —NR^{W4b}R^{W5b}, —C(═O)—O—R^{W6b}, —C(═O)—NH₂, —C(═O)—NHR^{W4b}, —C(═O)—NR^{W4b}R^{W5}, —C(═O)—NH—NH₂, —NH—C(═O)—R^{W6b}, Hetai 4, Hetcyc^{X}₄;

or R^{W1a} a and R^{W2a} or R^{W1b} and R^{W2b} form together a divalent

alkylene chain with 3 or 4 chain carbon atoms wherein 1 or 2 of non-adjacent CH₂ groups of the divalent alkylene chain may be replaced independently from each other by —N(H)—,

—N(C₁₋₆-alkyl)-, —N(—C(═O)—C₁₋₄-alkyl), —O— wherein that Ci-6-alkyl and Ci-4-alkyl radicals may be straight-chain or branched—, which divalent alkylene chain may be unsubstituted or mono- or di-substituted with independently from each other straight-chain or branched —Ci-6-alkyl;

Ai\hat{ }⁴ denotes a monocyclic aromatic ring system with 6 ring carbon atoms which ring system may be unsubstituted or monosubstituted with LA^{X4};

Hetar*⁴ denotes monocyclic aromatic ring system with 5 or 6 ring atoms wherein 1, 2, 3 or 4 of said ring atoms is/are a nitrogen atom(s) and the remaining are carbon atoms, wherein that aromatic ring system may be unsubstituted or mono-substituted with LA^{X4}, —NH₂, —NHR^{X7a}, —NR^{X7a}R^{XBa};

Hetar^{Y4} denotes a monocyclic aromatic ring system with 5 or 6 ring atoms wherein 1, 2 or 3 of said ring atoms is/are a nitrogen atom(s) and the remaining are carbon atoms, wherein that aromatic ring system may be unsubstituted or mono-substituted with LA^{Y4};

Hetcyc^{X4} denotes a saturated mono-cyclic heterocycle with 4, 5 or 6 ring atoms wherein 1 or 2 ring atom(s) is/are heteroatom(s) selected from N, O and/or S and the remaining ring atoms are carbon atoms, wherein that heterocycle may be unsubstituted or monosubstituted with LA^{X4} or —C(═O)-LA^{X4} or oxo (═O) or disubstituted with oxo (═O) and LA^{X4} or Hal and LA^{X4} or trisubstituted with one ot two Hal and one or two LA^{X4};

Hetcyc^{Y4} denotes a saturated mono-cyclic heterocycle with 4, 5 or 6 ring atoms wherein 1 or 2 ring atom(s) is/are heteroatom(s) selected from N, O and/or S and the remaining ring atoms are carbon atoms, wherein that heterocycle may be unsubstituted or monosubstituted with LA^{Y4} or —C(═O)-LA^{Y4} or oxo (═O) or disubstituted with oxo (═O) and LA^{Y4};

LA^{Xa} denotes straight-chain or branched Ci-6-alkyl which may be unsubstituted or mono-, di- or trisubstituted with independently from each other Hal, —CN, —NH₂, —NHR^{X7a}, —NR^{X7a}R^{X8a};

LA^{X4} and LA^{Y4} denote independently from each other straight-chain or branched C\hat{ }-alkyl;

LA^{Z4} denotes a straight-chain or branched divalent d-6-alkylene

radical;

_{R}w_{4aj R}w¾_{R}w_{6ai R}w_{4bj R}w_{5b) R}w_{6bi denote} independently from each other straight-chain or branched Ci-6-alkyl, a saturated monocyclic carbocycle with 3, 4, 5, 6, 7 carbon atoms, Ar*⁴, Hetar⁴, Hetcyc^{X4}, LA^{Z4}-Hetar^{Y4} or LA^{Z4}-Hetcyc^{Y4};

R^{X7a}, R^{X8a} denote independently from each other straight-chain or branched Ci-6-alkyl or a saturated monocyclic carbocycle with 3, 4, 5, 6, 7 carbon atoms or a monocyclic aromatic ring system with 5 or 6 ring atoms wherein 1, 2, 3 or 4 of said ring atoms is/are a nitrogen atom(s) and the remaining are carbon atoms, wherein that aromatic ring system may be unsubstituted or mono-substituted with straight-chain or branched C\hat{ }-alky!; or each pair R^{W4a} and R^{W5a}; R^{W4b} and R^{W5b}; R^{X7a} and R^{X8a} form together with the nitrogen atom to which they are attached to a 3, 4, 5, 6 or 7 membered heterocycle wherein that heterocyc!e may not contain any further heteroatom or may contain besides said nitrogen atom one further hetero ring atom selected from N, O and S, wherein, if that further hetero atom is N, that further N may be substituted with H or straight-chain or branched Ci-6-alkyl;

Hal denotes F, Cl, Br, I.

Compound according to item 5, or derivatives, N-oxides, prodrugs, solvates, tautomers or stereoisomers thereof as well as the

physiologically acceptable salts of each of the foregoing, including mixtures thereof in all ratios,

wherein Ar^{W4} denotes phenyl which is substituted with R^{w1a} in the ortho-position (relative to the attachment of Ar^{W4} to X) and bears no further substituent;

Hetar^{W4} denotes a monocyclic aromatic ring system with 6 ring atoms wherein 1 or 2 of said ring atoms is/are nitrogen atom(s) and the remaining are carbon atoms, wherein that ring system is substituted with R^{W1b} in the ortho-position (relative to the attachment of Hetar^{w4} to X) and bears no further substituent.

Compound according to item 5, or derivatives, N-oxides, prodrugs, solvates, tautomers or stereoisomers thereof as well as the

physiologically acceptable salts of each of the foregoing, including mixtures thereof in all ratios,

wherein Ar^{W4} denotes phenyl which is substituted with R^{w1a} in the ortho-position (relative to the attachment of Ar^{W4} to X) and bears one further substituent R^{W2a} in para-position relative to R^{wa};

Hetar^{W4} denotes a monocyclic aromatic ring system with 6 ring atoms wherein 1 or 2 of said ring atoms is/are nitrogen atom(s) and the remaining are carbon atoms, wherein that ring system is substituted with R^{wb} in the ortho-position (relative to the attachment of Hetar^{W4} to X) and bears one further substituent R^{W2b} in para-position relative to R^{W1b}.

Compound according to any one of items 5 to 7, or derivatives, N-oxides, prodrugs, solvates, tautomers or stereoisomers thereof as well as the physiologically acceptable salts of each of the foregoing, including mixtures thereof in all ratios,

wherein R^{w1a}, R^{w1b} denote independently from each other methyl,

methylaminomethyl, (dimethylamino)methyl, pyrazolyl, methyl pyrazolyl, imidazolyl, methylimidazolyl, 1-methyl-1H-imidazol-4-yl, pyrimidinyl, tetrazolyl, 1H-1,2,3,4-tetrazol-5-yl, Cl, —CN, —S0₂NH₂, —S0₂NH(CH₃), —S0₂N(CH₃)₂, —S0₂-N-morpholinyl, —S0₂-N-piperazinyl, —S0₂-CH₃, —S0₂-NH-pyrrolidinyl, —S0₂-NH-pyrrolidin-3-yl, —S0₂-NH-methylpyrrolidinyl, —S0₂—NH-(1-methylpyrrolidin-3-yl), —S0₂-NH-(piperdinyl), —S0₂-NH-(piperdin-3-yl), —S0₂-NH-(methylpiperdinyl), —S0₂—NH-(1-methylpiperdin-3-yl), —S0₂-NH-oxanyl, —S0₂-NH-oxan-3-yl, —S0₂—NH—CH₂-(pyrrolidinyl), —S0₂-NH—CH₂-(pyrrolidin-3-yl), —S0₂—NH—CH₂-(methylpyrrolidinyl), —S0₂—NH—CH₂—(1-methylpyrrolidin-3-yl), —S0₂-NH—CH₂-oxanyl, —S0₂-NH—CH₂-oxan-4-yl, —S0₂—NH—CH₂-pyrazolyl, —S0₂—NH—CH₂-pyrazol-4-yl, —S0₂—NH—CH₂-

(methylpyrazolyl), —SO₂—NH—CH₂—(1-methyl-1H-pyrazol-4-yl), —SO₂—NH-(pyrimidin-5-yl), —SO₂—NH—CH₂-(pyrimidin-5-yl), —SO₂—N(CH₃)-CH₂-(pyrimidin-5-yl), —NH₂, —N-piperazinyl, —N-4-methylpiperazinyl, 4-N-acetylpiperazin-1-yl, —OH, —OCH₃, —C(═O)—OH, —C(═O)—O-(n-C₄H₉), —C(═O)—O-pyrimidinyl, —C(═O)—O-pyrimidin-4-yl, —C(═O)—O-(aminopyrimidinyl), —C(═O)—O—(2-aminopyrimidin-4-yl), —C(═O)—NH₂) —C(═O)—NHCH₃, —C(═O)-N(CH₃)₂) —C(═O)—NH-cyclohexyl, —C(═O)—NH-phenyl, —C(═O)—NH-(azetidinyl), —C(═O)—NH-(methylazetidinyl), —C(═O)—NH—(1-methylazetidin-3-yl), —C(═O)—NH—(1-acetylazetidin-3-yl), —C(═O)—NH—CH₂-(azetidinyl), —C(═O)-NH—CH₂—(1-acetylazetidin-3-yl), —C(═O)—NH-(methylpyrrolidinyl), —C(═O)—NH—(1-methy!-pyrrolidin-3-yl), —C(═O)—NH—((3S)-1-methyl-pyrrolidin-3-yl), —C(═O)—NH—((3yl)-1-methyl-pyrrolidin-3-yl), —C(═O)—N(CH₃)-(methylpyrrolidinyl), —C(═O)—N(CH₃)-(1-methyl-pyrrolidin-3-yl), —C(═O)—NH—CH₂-(methylpyrrolidinyl), —C(═O)-NH—CH₂—(1-methyl-pyrrolidin-3-yl), —C(═O)—NH—(1-acetylpyrrolidin-3-yl), —C(═O)—NH-(fluoro-methylpyrrolidinyl), —C(═O)—NH—(2-fluoro-1-methylpyrrolidin-3-yl), —C(═O)—NH—(5-fluoro-1-methylpyrrolidin-3-yl), —C(═O)—NH-(difluoro-methylpyrrolidinyl), —C(═O)—NH—(5,5-difluoro-1-methylpyrrolidin-3-yl), —C(═O)—NH—(3,3-difluoro-1-methylpyrrolidin-3-yl),

—C(═O)—NH-oxanyl, —C(═O)—NH-oxan-4-yl, —C(═O)—NH-piperidinyl, —C(═O)—NH-piperidin-4-yl, —C(═O)—NH-piperidin-3-yl, —C(═O)—NH-methylpiperidinyl, —C(═O)—NH—(1-methylpiperidin-4-yl), —C(═O)—NH—(1-methylpiperidin-3-yl), —C(═O)—NH-(acetylpiperdinyl), —C(═O)—NH—(1-acetylpiperidin-3-yl), —C(═O)—NH—(1-acetylpiperidin-4-yl), —C(═O)—NH-(oxopyrrolidinyl), —C(═O)—NH—(N-methyl-oxopyrrolidinyl), —C(═O)—NH—(5-oxopyrrolidin-3-yl), —C(═O)—NH—(2-oxopyrrolidin-3-yl), —C(═O)—NH—(1-methyl-5-oxopyrrolidin-3-yl), —C(═O)—NH—(1-methyl-2-oxopyrrolidin-3-yl), —C(═O)—NH-morpholinyl, —C(═O)—NH—CH₂-morpholinyl, —C(═O)—NH—CH₂—

morpholin-2-yl, —C(═O)—NH—CH₂-morpholin-3-yl, —C(═O)—NH—CH₂-(methylmorpholinyl), —C(═O)—NH—CH₂-(4-methylmorpholin-2-yl),—C(═O)—NH—CH₂-(acetylmorpholinyl). —C(═O)—NH—CH₂—(4-acetylmorpholin-2-yl), —

—C(=0)-NH—CH₂—(4-acetylmorpholin-3-yl),—C(=0)-NH-(oxopiperidinyl), —C(=0)-NH—(2-oxopiperidin-4-yl), —C(=0)-NH-(methyl-oxopiperidinyl), —C(=0)-NH—(1-methyl-2-oxopiperidin-4-yl), —C(=0)-NH—(1-methyl-6-oxopiperidin-3-yl), —C(=0)-NH(pyrimindin-4-yl), —C(=0)-NH(pyrimindin-5-yl), —C(=0)-NHCH2(pyrimindin-5-yl)″, —C(=0)-NH-imidazolyl, —C(=0)-NH-imidazo!-5-yl, —C(=0)-NH-methylimidazolyl, —C(=0)-NH—(1-methyl-imidazol-5-yl), —C(=0)-NH—CH₂-imidazolyl, —C(=0)-NH—CH₂-imidazol-5-yl, —C(=0)-NH—CH₂-(methylimidazolyl), —C(=0)-NH—CH₂—(1-methyl-1H-imidazol-5-yl), —C(=0)-NH(methylpyrazolyl), —C(=0)-NH(1-methyl-1H-pyrazol-4-yl), —C(=0)-NHCH2(1-methylpyrazol-4-yl), —C(=0)-NH₂-pyridinyl, —C(=0)-NH₂-pyridin-3-yl, —C(=0)-NH-pyridazinyl,—C(=0)-NH-pyridazin-3-yl, —C(=0)-NH—CH₂-pyridazinyl, —C(=0)-NH—CH₂-pyridazin-3-yl, —C(=0)-NH-pyrimidinyl, —C(=0)-NH-pyrimidin-4-yl, —C(=0)-NH-pyrimidin-5-yl, —CH₂-NH-(pyrimidin-5-yl);

R^{W2a}, R^{W2b} denote, if present, independently from each other H, Br, —CH₂NH₂, —CN, —NO₂, —NH₂, —NH-C(═O)—CH₃, —C(═O)—O-methyl, —C(═O)—NH₂, —C(═O)—NH—NH₂, 4-methylpiperazin-1-yl, 4-acetylpiperazin-1-yl, methylpyrazolyl, 1-methyl-1H-pyrazol-5-yl, 1H-imidazol-1-yl, oxazolyl, 1,3-oxazol-2-yl, 2H-1,2,3,4-tetrazol-5-yi;

or R^{wb} and R^{W2b} form together a divalent -0-CH₂—CH₂-NH-chain it being understood that the the oxygen atom of that chain is attached to the Hetar^{W4} substituent at the position of R^{w1b} while the —NH— part of that chain is attached to the Hetar^{W4} substituent at the position of R^{W2b} and next to R^{w1b}.

Compound according to any one of items 5 to 7, or derivatives, N-oxides, prodrugs, solvates, tautomers or stereoisomers thereof as well as the physiologically acceptable salts of each of the foregoing, including mixtures thereof in all ratios,

wherein Ar^{W4} denotes 2-((dimethylamino)methyl)phenyl, 2-(C(=0)OH)phenyl, 2-methylsulfonylphenyl (2-methanesulfonylphenyl), 2-(morpholine-4-sulfonyl)phenyl, 2-hydroxyphenyl, 2-methoxyphenyl, 2-cyanophenyl, 2-aminosulfonylphenyl, 2-(N-methylaminosulfonyl)phenyl, 2-((1-methylpyrrolidin-3-yl)-NH-S0₂-)phenyl, 2-((1-methylpiperidin-3-yl)-NH-S0₂-)phenyl, 2-((oxan-3-yl)-NH-S0₂-)phenyl, 2-((1-methylpyrrolidin-3-yl)-CH₂-NH-S0₂-)phenyl, 2-(oxan-4-yl-CH₂-NH-S0₂-)phenyl, 2-((1-methyl-1H-pyrazol-4-yl)-CH₂-NH-S0₂-)phenyl, 2-((pyrimidin-5-yl)-CH₂-NH-S0₂-)phenyl, 2-((pyrimidin-5-yl)-CH₂-N(CH₃)-S0₂-)phenyl, 2-(ty/v″-dimethylaminosulfonyl)phenyl, 2-(NH₂—C(=0)-)phenyl (2-carbamoylphenyl), 2-((1-methylpyrrolidin-3-yl)-NH-C(=0)-)phenyl, 5-bromo-2-methanesulfonylphenyl, 2-(piperazine-1-sulfonyl)phenyl, 5-cyano-2-methanesulfonylphenyl, 2-methanesulfonyl-5-amino-phenyl, 2-methanesulfonyl-5-nitro-phenyl, 2-methanesulfonyl-5-aminomethyl-phenyl, 2-methanesulfonyl-5-carbamoylphenyl (2-methanesulfonyl-5-(NH₂-C(=0)-)phenyl), (2-methanesulfonyl-5-(NH₂-NH—C(=0)-)phenyl), 2-methanesulfonyl-5-(CH₃C(=0)NH)-phenyl, 2-methanesulfonyl-5-(4-acetylpiperazin-1-yl)-phenyl, 2-methanesulfonyl-5-(4-methylpiperazin-1-yl)-phenyl, 2-methanesulfonyl-5-(1,3-oxazol-2-yl)phenyl, methanesulfonyl-5-(2H-1,2,3,4-tetrazol-5-yl)phenyl, 5-(1-/-imidazol-1-yl)-2-methanesulfonylphenyl;

Hetar^{W4} denotes 4-(methylamino)methylpyridin-3-yl, 4-((dimethylamino)methyl)pyridin-3-yl, 2-methylsulfonylpyrdin-3-yl, 4-methylsulfonylpyridin-3-yl, 2-aminopyridin-3-yl, 4-(NH₂—C(=0))-pyridin-3-yl, 4-chloropyridin-3-yl, 4-cyanopyridin-3-yl, 2-hydroxy-pyridin-3-yl, 2-methoxy-pyridin-3-yl, 3-methanesulfonyl-pyrazin-2 yl, 3-methanesulfonyl-pyridin-2-yl, 4-(C(=0)OH)pyridin-3-yl, 4-(1-methyl-1-pyrazol-4-yl)-pyridin-3-yl, 4-(4-methylpiperazin-1-yl)-pyridin-3-yl, 4-(4-N-acetylpiperazin-1-yl)pyridin-3-yl, 4-(1-methyl-1H-imidazol-4-yl)pyridin-3-yl, 4-(pyrimidin-5-yl)-pyridin-3-yl, 4-methoxypyridin-3-yl, 4-(1H-1,2,3,4-tetrazol-5-yl)pyridin-3-yl, 4-((2 aminopyrimidin-4-yl)-O—C(=0))-pyridin-3-yl, 4-(CH₃NH—C(=0))-pyridin-3-yl, 4-((CH₃)₂N—C(=0))-pyridin-3-yl, 4-((-(1-methyl-azetidin-3-yl)-NH—C(=0)-)pyridin-3-yl, 4-((1-acetylazetidin-3-yl)-NH—C(=0)-)pyridin-3-yl, 4-((1-methylpyrrolidin-3-yl)-NH—C(=0)-)pyridin-3-yl (4-(1-methylpyrrolidin-3-ylcarbamoyl)pyridin-3-yl), 4-((1-methylpyrrolidin-3-yl)-N(CH₃)-C(=0)-)pyridin-3-yl, 4-(1-methyl-pyrrolidin-3-yl)-CH₂-NH—C(=0)-pyridin-3-yl (4-(1-methyl-pyrrolidin-3-ylmethylcarbamoyl)pyridin-3-yl), 4-(1-acetyl pyrrol id in 3-yl)-NH—C(=0)-pyridin-3-yl, 4-(5-fluoro-1-methylpyrrolidin-3-yl)-NH—C(=0)-pyridin-3-yl, 4-(3-fluoro-1-methylpyrrolidin-3-yl)-NH—C(=0)-pyridin-3-yl, 4-(5,5-difluoro-1-methylpyrrolidin-3-yl)-NH—C(=0)-pyridin-3-yl, 4-(3,3-difluoro-1-methylpyrrolidin-3-yl)-NH-C(=0)-pyridin-3-yl, 4-(oxan-4-yl-NH—C(=0))pyridin-3-yl, 4-((1-methylpiperidin-4-yl)-NH—C(=0)-)pyridin-3-yl (4-(1-methyl-piperidin-4-ylcarbamoyl)pyridin-3-yl), 4-((1-methylpiperidin-3-yl)-NH—C(=0)-)pyridin-3-yl (4-(1-methylpiperidin-3-ylcarb-amoyl)pyridin-3-yl), 4-(((3S)-1-methyl-pyrrolidin-3-yl)-NH—C(=0)-)pyridin-3-yl, 4-(((3R)-1-methyl-pyrrolidin-3-yl)-NH—C(=0)-)pyridin 3-yl, 4-(1-acetylpiperidin-3-ylcarbamoyl)pyridin-3-yl, 4-(1-acetylpiperidin-4-ylcarbamoyl)pyridin-3-yl, 4-(1-acetylpiperidin-3-ylmethylcarbamoyl)pyridin-3-yl, 4-(1-acetylpiperidin-4-ylmethylcarbamoyl)pyridin-3-yl, 4-((1-acetylazetidin-3-yl)-CH₂-NH—C(=0)-)pyridin-3-yl (4-(1-acetylazetidin-3-ylmethylcarbamoyl)pyridin-3-yl), 4-(5-oxopyrrolidin-3-yl)-NH—C(=0)-pyridin-3-yl, 4-(2-oxopyrrolidin-3-yl)-NH—C(=0)-pyridin-3-yl, 4-(1-methyl-5-oxopyrrolidin-3-yl)-NH—C(=0)-pyridin-3-yl, 4-(1-methyl-2-oxopyrrolidin-3-yl)-NH—C(=0)-pyridin-3-yl, 4-(morpholin 3-yl)-CH₂-NH—C(=0)-pyridin-3-yl, 4-(4-methylmorpholin-2-yl)-CH; NH-CO-pyridin-3-yl, (4-acetylmorpholin-3-yl)-CH₂-NH—C(=0)-pyridin-3-yl, 4-acetylmorpholin-2-yl-CH₂-NH—C(=0)-pyridin-3-yl (4-acetylmorpholin-2-ylmethylcarbamoylpyridin-3-yl), 4-((2-oxopiperidin-4-yl)-NH—C(=0)-)pyridin-3-yl (4-(2-oxopiperidin-4-ylcarbamoyl)pyridin-3-yl), 4-((1-methyl-2-oxopiperidin-4-yl)-NH-C(=0)-)pyridin-3-yl (4-(1-methyl-2-oxopiperidin-4-ylcarbamoyl)pyridin-3-yl), 4-(1-methyl-6-oxopiperidin-3-yl)-NH—C(=0)-)pyridin-3-yl (4-(1-methyl-6-oxopiperidin-3-ylcarbamoyl)pyridin-3-yl, 4-(phenyl-NH—C(=0)-)pyridin-3-yl (4-(phenylcarbamoyl)pyridin-3-yl), 4-((1-methyl-1H-pyrazol-4-yl)NH-C(=0))pyridin-3-yl, 4-((1-methylpyrazol-4-yl)-CH₂NH—C(=0))-pyridin-3-yl, 4-(pyridin-3-yl)-NH—C(=0)-pyridin-4-yl, 4-((1-methyl-imidazol-5-yl)-CH₂-NH—C(=0)-)pyridin-3-yl) (4-(1-methyl-imidazol 5-ylmethyl)carbamoylpyridin-3-yl), 4-((pyrimidin-4-yl)-NH—C(=0))pyridin-3-yl, 4-((pyrimidinyl-5-yl)-NHC(=0))-pyridin-3-yl, 4-((pyrimidinyl-5-yl)-CH₂NHC(=0))-pyridin-3-yl, 4-(pyridazin-3-ylmethylcarbamoyl)pyridin-3-yl, 4-methanesulfonyl-pyridin-1-ium-1-olate-3-yl, 2Hi3H,4H-pyrido[4,3-b][1₁4]oxazin-8-yl, 4-carbamoylpyrimidin-5-yl, I-methyl-I H-I \hat{ }.S-triazol-S-yl, 4-[(pyrimidin-5-yl)amino]rnethylpyridin-3-yl.

10. Compound according to item 9, or derivatives, N-oxides, prodrugs, solvates, tautomers or stereoisomers thereof as well as the physiologically acceptable salts of each of the foregoing, including mixtures thereof in all ratios, wherein R denotes 4-ethylphenyl, 2,5-dimethylphenyl, 3-methoxyphenyl, 4-fluorophenyl, 3-bromophenyl, 4-bromophenyl, 2-chloro-5-methoxy-phenyl, 3-amino-4-methylphenyl, 4-amino-3-fluoro-phenyl, dihydrobenzofuran-5-yl, A-methyl-indol-6-yl, 1-ethyl-1H-indol-6-yl, 2-(difluoromethyl)-1H-indol-6-yl, 1,4-dimethyl-1H-indol-6-yl, 1,5-dimethyl-1H-indol-6-yl, 4-fluoro-1-methyl indol-6-yl, 5-fluoro-1-methylindol-6-yl, 7-fluoro-1-methyl-indol-6-yl, benzothiazol-6-yl, benzothiazol-5-yl, 3-methyl-1-benzofuran-5-yl, 3-methyl-1-benzothiophen-5-yl, 2,3-dihydrobenzo[1,4]dioxin-6-yl,

1-methyl-1/- -pyrrolo[2,3-b]pyrdin-6-yl, 2-amino-1,3-benzothiazol-5-yl, 2-amino-1,3-benzothiazol-6-yl, 2-(pyrrolidin-2-yl-C(═O)—NH—)-1,3-benzothiazol-6-yl, 2,1,3-benzothiadiazol-5-yl.

11. Compound according to any one of items 1 to 10, or derivatives, N-oxides and/or physiologically acceptable salts thereof, selected from the group consisting of:
8-(2,3-dihydro-1,4-benzodioxin-6-yl)-A/-(4-methanesulfonylpyridin-3-yl)quinoxalin-6-amine
5-(1-methyl-1H-indol-6-yl)-7-{1H,2H,3H-pyrrolo[2,3-c]pyridin-1-yl}quinoxaline A-(2-methanesulfonylphenyl)-8-(1-methyl-1H-indol-6-yl)quinoxalin-6-amine
8-(1,3-benzothiazol-6-yl)-A/-(4-methanesulfonylpyridin-3-yl)quinoxalin-6-amine
8-(2-chloro-5-methoxyphenyl)-A/-(4-methanesulfonylpyridin-3-yl)quinoxalin-6-amine
A/-(2-methanesulfonylpyridin-3-yl)-8-(1-methyl-1-indol-6-yl)quinoxalin-6-amine
8-(1-methyl-1H-indol-6-yl)-A/-[2-(morpholine-4-sulfonyl)phenyl]quinoxalin-6-amine
2-{[8-(1-methyl-1-indol-6-yl)quinoxalin-6-yl]amino}benzene-1-sulfonamide
8-(1,3-benzothiazol-5-yl)-A/-(4-methanesulfonylpyridin-3-yl)quinoxalin-6-amine trifluoroacetate
A/-(5-bromo-2-methanesulfonylphenyl)-8-(1-methyl-1H-indol-6-yl)quinoxalin-6-amine
A-(4-methanesulfonylpyridin-3-yl)-8-(1-methyl-1H-indol-6-yl)quinoxalin-6-amine
A/-(2-methoxypyridin-3-yl)-8-(1-methyl-1-indol-6-yl)quinoxalin-6-amine
3-{[8-(1-methyl-1-indol-6-yl)quinoxalin-6-yl]amino}pyridin-2-ol
8-(1-methyl-1/-/-indol-6-yl)-A-[2-(piperazine-1-sulfonyl)phenyl]quinoxalin 6-amine
A/-methyl-2-{[8-(1-methyl-1/-/-indol-6-yl)quinoxalin-6-yl]amino}benzene-1-sulfonamide
3-N-[8-(1-methyl-1-indol-6-yl)quinoxalin-6-yl]pyridine-2,3-diamine
3-{[8-(1-methyl-1-indol-6-yl)quinoxalin-6-yl]amino}pyridine-4-carbonitrile
3-{[8-(1-methyl-1-indol-6-yl)quinoxalin-6-yl]amino}pyridine-4-carboxamide
A/,A/-dimethyl-2-{[8-(1-methyl-1H-indol-6-yl)quinoxalin-6-yl]amino}benzene-1-sulfonamide
/S-(2-methanesulfonylphenyl)-8-{1-methyl-1-pyrrolo[2,3-b]pyridin-6-yl}quinoxalin-6-amine trifluoroacetate
A-(4-methanesulfonylpyridin-3-yl)-8-(3-methyl-1-benzofuran-5-yl)quinoxalin-6-amine
∧-(4-methoxypyridin-3-yl)-8-(1-methyl-1-indol-6-yl)quinoxalin-6-amine
3-{[8-(3-methyl-1-benzofuran-5-yl)quinoxalin-6-yl]amino}pyridine-4-carbonitrile
4-methanesulfonyl-3-{[8-(1-methyl-1H-indol-6-yl)quinoxalin-6-yl]amino}benzonitrile
3-{[8-(3-methyl-1-benzofuran-5-yl)quinoxalin-6-yl]amino}pyridine-4-carboxamide
A-(5-methanesulfonylpyrimidin-4-yl)-8-(1-methyl-1H-indol-6-yl)quinoxalin-6-amine
3-{[8-(1-methyl-1H-indol-5-yl)quinoxalin-6-yl]amino}pyridine-4-carbonitrile
3-{[8-(1-methyl-1/- -indol-5-yl)quinoxalin-6-yl]amino}pyridine-4-carboxamide
A/-(4-chloropyridin-3-yl)-8-(1-methyl-1H-indol-5-yl)quinoxalin-6-amine 8-(1-methyl-1H-indol-5-yl)-A/-[4-(1-methyl-1H-pyrazol-4-yl)pyridin-3-yl]quinoxalin-6-amine
8-(1-methyl-1-indol-5-yl)-A-[4-(4-methylpiperazin-1-yl)pyridin-3-yl]quinoxalin-6-amine
8-(1-methyl-1H-indol-5-yl)-A/-[4-(pyrimidin-5-yl)pyridin-3-yl]quinoxalin-6-amine
5-(1-methyl-1H-indol-5-yl)-7-{1H,2 V,3H-pyrrolo[2,3-c]pyridin-1-yl}quinoxaline A/-(2-methanesulfonyl-5-nitrophenyl)-8-(1-methylindol-6-yl)quinoxalin-6-amine
6-methanesulfonyl-/S/1-[8-(1-methyl-1-indol-6-yl)quinoxalin-6-yl]benzene-1,3-diamine
8-(2,3-dihydro-1-benzofuran-5-yl)-A-(4-methanesulfonylpyridin-3-yl)quinoxalin-6-amine
A/-[5-(aminomethyl)-2-methanesulfonylphenyl]-8-(1-methyl-1H-indol-5-yl)quinoxalin-6-amine
8-(2,5-dimethylphenyl)-N-(4-methanesulfonylpyridin-3-yl)quinoxalin-6-amine
8-(1-methyl-1H-indol-6-yl)-A-[4-(4-methylpiperazin-1-yl)pyridin-3-yl]quinoxalin-6-amine
A/-(4-methanesulfonyl-3-{[8-(1-methyl-1-indol-6-yl) quinoxalin-6-yl]amino}phenyl)acetamide
A-[5-(1-/-imidazol-1-yl)-2-methanesuifonylphenyl]-8-(1-methyl-1H-indol-6-yl)quinoxalin-6-amine
A-[2-methanesulfonyl-5-(2H-1,2,3,4-tetrazol-5-yl)phenyl]-8-(1-methyl-1-indol-6-yl)quinoxalin-6-amine
4-methanesulfonyl-3-{[8-(1-methyl-1/-/-indol-6-yl)quinoxalin-6-yl]amino}pyridin-1-ium-1-olate A/-[2-methanesulfonyl-5-(4-methylpiperazin-1-yl)phenyl]-8-(1-methyl-1H-indol-6-yl)quinoxalin-6-amine
1-[4-(4-methanesulfonyl-3-{[8-(1-methyl-1H-indol-6-yl)quinoxalin-6-yl]amino}phenyl)piperazin-1-yl]ethan-1-one
3-{[8-(1-methyl-1H-indol-6-yl)quinoxalin-6-yl]oxy}pyridine-4-carbonitrile A7-(4-methanesuifonylpyridin-3-yl)-8-[3-(1H-1,2,3-triazol-4-yl)phenyl]quinoxalin-6-amine
/S/-(4-methanesulfonylpyridin-3-yl)-8-[1-(propan-2-yl)-1-indol-6-yl]quinoxalin-6-amine
8-[3-(dimethylamino)phenyl]-A/-(4-methanesulfonylpyridin-3-yl)quinoxalin-6-amine
A-(4-methanesulfonylpyridin-3-yl)-8-(3-methylphenyl)quinoxalin-6-amin/-methyl-3-{[8-(1-methyl-1H-indol-6-yl)quinoxalin-6-yl]amino}pyridine-4-carboxamide
A,A7-dimethyl-3-{[8-(1-methyl-1H-indol-6-yl)quinoxalin-6-yl]amino}pyridine-4-carboxamide
3-{[8-(1-methyl-1-indol-6-yl)quinoxalin-6-yl]amino}-A/-(pyrimidin-5-y I)pyridine-4-carboxamide
3-{[8-(1-methyl-1H-indol-6-yl)quinoxalin-6-yl]amino}-N-(pyrimidin-5-ylmethyl)pyridine-4-carboxamide
3-{[8-(1-methyl-1H-indol-6-yl)quinoxalin-6-yl]amino}-∧-[(1-methyl-1H-pyrazol-4-yl)methyl]pyridine-4-carboxamide
4-methanesulfonyl-\1-methyl-/V3-[8-(1-methyl-1H-indol-6-yl)quinoxalin-6-yl]benzene-,3-diamine
8-[3-(chloromethyl)-1-benzofuran-5-yl]-N-(4-methanesulfonylpyridin-3-yl)quinoxalin-6-amine
8-(7-fluoro-1-methyl-1H-indol-6-yl)-N-(4-methanesulfonylpyridin-3-yl)quinoxalin-6-amine
8-(4-ethylphenyl)-N-(4-methanesulfonylpyridin-3-yl)quinoxalin-6-amine 8-(1H-1,3-benzodiazol-5-yl)-N-(4-methanesulfonylpyridin-3-yl)quinoxalin-6-amine
N-(4-methanesulfonylpyridin-3-yl)-8-(3-methoxyphenyl)quinoxalin-6-amine
8-(3,3-dimethyl-2,3-dihydro-1-benzofuran-5-yl)-N-(4-methanesulfonylpyridin-3-yl)quinoxalin-6-amine
8-(3-ethylphenyl)-N-(4-methanesulfonylpyridin-3-yl)quinoxalin-6-amine
8-(2-amino-5-methylphenyl)-N-(4-methanesulfonylpyridin-3-yl)quinoxalin-6-amine
2-{7-[(4-methanesulfonylpyridin-3-yl)amino]quinoxalin-5-yl}-4-methylphenol
8-(1-methyl-1H-indol-6-yl)-N-[4-(1H-1 \hat{ }.S\hat{ }-tetrazol-S-y pyridin-S-yl]quinoxalin-6-amine
N-(4-chloropyridin-3-yl)-8-(1-methyl-1H-indol-6-yl)quinoxalin-6-amine 8-(4-fluoro-1-methyl-1H-indol-6-yl)-N-(4-methanesulfonylpyridin-3-yl)quinoxalin-6-amine
4-methanesulfonyl-3-{[8-(3-methyl-1-benzofuran-5-yl)quinoxalin-6-yl]amino}pyridin-1-ium-1-olate
8-(5-fluoro-1-methyl-1H-indol-6-yl)-N-(4-methanesulfonylpyridin-3-yl)quinoxalin-6-amine
N-(4-methanesulfonylpyridin-3-yl)-8-(2-methoxy-5-methylphenyl)quinoxalin-6-amine
8-(3-amino-4-methylphenyl)-N-(4-methanesulfonylpyridin-3-yl)quinoxalin-6-amine
N-(3-methanesulfonylpyridin-2-yl)-8-(1-methyl-1H-indol-6-yl)quinoxalin-6-amine
1-[4-(3-{[8-(1-methyl-1H-indol-6-yl)quinoxalin-6-yl]amino}pyridin-4-yl)piperazin-1-yl]ethan-1-one
N-[4-(1-methyl-1H-imidazol-4-yl)pyridin-3-yl]-8-(1-methyl-1H-indol-6-yl)quinoxalin-6-amine
8-(1-methyl-1H-indol-e-y-N\hat{ }H.SH\hat{ }H-pyrido\hat{ }.S-\hat{ }tl.\hat{ }oxazin-S-yl}quinoxalin-6-amine
2-{[8-(1-methyl-1H-indol-6-yl)quinoxalin-6-yl]amino}-N-[(pyrimidin-5-yl)methyl]benzene-1-sulfonamide
2-{[8-(1-methyl-1H-indol-6-yl)quinoxalin-6-yl]amino}benzonitrile
2-{[8-(1-methyl-1H-indol-6-yl)quinoxalin-6-yl]amino}benzamide
4-cyano-3-{[8-(1-methyl-1H-indol-6-yl)quinoxalin-6-yl]amino}pyridin-1-ium-1-olate
3-{methyl[8-(1-methyl-1H-indol-6-yl)quinoxalin-6-yl]amino}pyridine-4-carbonitrile
3-{[8-(1-methyl-1H-indol-6-yl)quinoxalin-6-yl]amino}-N-(1-methyl-1H-pyrazol-4-yl)pyridine-4-carboxamide
N-[2-methanesulfonyl-5-(1-methyl-1H-pyrazol-5-yl)phenyl]-8-(1-methyl-1H-indol-6-yl)quinoxalin-6-amine
N-[2-methanesulfonyl-5-(1,3-oxazol-2-yl)phenyl]-8-(1-methyl-1H-indol-6-yl)quinoxalin-6-amine
3-{methyl[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-phenylpyridine-4-carboxamide
3-{[8-(1-methyl-1H-indol-6-yl)quinoxalin-6-yl]amino}-N-(1-methyl-2-oxopiperidin-4-yl)pyridine-4-carboxamide
N-(1-acetylazetidin-3-yl)-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-(1-methylpyrrolidin-3-yl)pyridine-4-carboxamide
2-{[8-(1-methyl-1H-indol-6-yl)quinoxalin-6-yl]amino}-N-(pyrimidin-5-yl)benzene-1-sulfonamide
3-{[8-(1-methyl-1H-indol-6-yl)quinoxalin-6-yl]amino}-N-(oxan-4-yl)pyridine-4-carboxamide
6-methanesulfonyl-N1-[8-(3-methyl-1-benzofuran-5-yl)quinoxalin-6-yl]benzene-1,3-diamine
N-(2-methanesulfonyl-5-nitrophenyl)-8-(3-methyl-1-benzofuran-5-yl)quinoxalin-6-amine
N-(4-methanesulfonylpyridin-3-yl)-N-methyl-8-(1-methyl-1H-indol-6-yl)quinoxalin-6-amine
N-(4-methanesulfonylpyridin-3-yl)-8-(3-methyl-1-benzothiophen-5-yl)quinoxalin-6-amine
Methyl 4-methanesulfonyl-3-{[8-(1-methyl-1H-indol-6-yl)quinoxalin-6-yl]amino}benzoate
4-Methanesulfonyl-3-{[8-(1-methyl-1H-indol-6-yl)quinoxalin-6-yljaminojbenzamide
8-(2, 1,3-benzothiadiazol-5-yl)-N-(4-methanesulfonylpyridin-3-yl)quinoxalin-6-amine
8-(1H-1,2,3-benzotriazol-5-yl)-N-(4-methanesulfonylpyridin-3-yl)quinoxalin-6-amine
4-methanesulfonyl-3-{[8-(1-methyl-1H-indol-6-yl)quinoxalin-6-yl]amino}benzohydrazlde
8-(2, 1,3-benzoxadiazol-5-yl)-N-(4-methanesulfonylpyridin-3-yl)quinoxalin-6-amine
N-(1-acetylpyrrolidin-3-yl)-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-(1-methyl-6-oxopiperidin-3-yl)pyridine-4-carboxamide
3-{[8-(1-methyl-1H-indol-6-yl)quinoxalin-6-yl]amino}-N-(1-methylpiperidin-4-yl)pyridine-4-carboxamide
3-{[8-(1-methyl-1H-indol-6-yl)quinoxalin-6-yl]amino}-N-(1-methylpiperidin-3-yl)pyridine-4-carboxamide
3-{methyl[8-(1-methyl-1H-indol-6-yl)quinoxalin-6-yl]amino}-N-(pyrimidin-5-yl)pyridine-4-carboxamide
N-cyclohexyl-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-(2-oxopiperidin-4-yl)pyridine-4-carboxamide
2-{7-[(4-methanesulfonylpyridin-3-yl)amino]quinoxalin-5-yl}-4-methylbenzamide
8-(3-ethoxyphenyl)-N-(4-methanesulfonylpyridin-3-yl)quinoxalin-6-amine
N-(4-methanesulfonylpyridin-3-yl)-8-[3-(propan-2-yloxy)phenyl]quinoxalin-6-amine
8-(4-aminophenyl)-N-(4-methanesulfonylpyridin-3-yl)quinoxalin-6-amine 8-(3-aminophenyl)-N-(4-methanesulfonylpyridin-3-yl)quinoxalin-6-amine butyl 3-{[8-(1-methyl-1H-indol-6-yl)quinoxalin-6-yl]amino}pyridine-4-carboxylate
3-{[8-(1-methyl-1H-indol-6-yl)quinoxalin-6-yl]amino}-N-[(morpholin-3-yl)methyl]pyridine-4-carboxamide
N-[(4-acetylmorpholin-3-yl)methyl]-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-[(4-methylmorpholin-2-yl)methyl]pyridine-4-carboxamide
N-[(-acetylazetidin-3-yl)methyl]-3-{[8-(1-methyl-1H-indol-6-yl)quinoxalin-6-yl]amino}pyridine-4-carboxamide
N-[(4-acetylmorpholin-2-yl)methyl]-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-[(1-methylpyrrolidin-3-yl)methyl]pyridine-4-carboxamide
N-[(1-methyl-1H-imidazol-5-yl)methyl]-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-[(pyridazin-3-yl)methyl] pyridine-4-carboxamide
4-{[8-(1-methyl-1H-indol-6-yl)quinoxalin-6-yl]amino}pyridine-3-carbonitrile
N-(1-acetylpiperidin-4-yl)-3-{[8-(1-methyl-1H-indol-6-yl)quinoxalin-6-yl]amino}pyridine-4-carboxamide
N-(1-acetylpiperidin-3-yl)-3-{[8-(1-methyl-1H-indol-6-yl)quinoxalin-6-yl]amino}pyridine-4-carboxamide
5-{[8-(1-methyl-1H-indol-6-yl)quinoxalin-6-yl]amino}pyrimidine-4-carboxamide
3-{[8-(3-methyl-1-benzothiophen-5-yl)quinoxalin-6-yl]amino}pyridine-4-carbonitrile
3-{[8-(3-methyl-1-benzothiophen-5-yl)quinoxalin-6-yl]amino}-N-(1-methylpyrrolidin-3-yl)pyridine-4-carboxamide
N-(4-methanesulfonylpyridin-3-yl)-8-(4-methoxyphenyl)quinoxalin-6-amine
N-(4-methanesulfonylpyridin-3-yl)-8-(5-methoxy-2-methylphenyl)quinoxalin-6-amine
8-[1-(difluoromethyl)-1H-indol-6-yl]-N-(4-methanesulfonylpyridin-3-yl)quinoxalin-6-amine
8-(4-bromophenyl)-N-(4-methanesulfonylpyridin-3-yl)quinoxalin-6-amine 8-(3-bromophenyl)-N-(4-methanesulfonylpyridin-3-yl)quinoxalin-6-amine 2-aminopyrimidin-4-yl 3-{[8-(1-methyl-1H-indol-6-yl)quinoxalin-6-yl]amino}pyridine-4-carboxylate
8-(1,2-benzothiazol-5-yl)-N-(4-methanesulfonylpyridin-3-yl)quinoxalin-6-amine
8-(2-amino-1,3-benzothiazol-5-yl)-N-(4-methanesulfonylpyridin-3-yl)quinoxalin-6-amine
N-(4-methanesulfonylpyridin-3-yl)-8-[3-(trifluoromethoxy)phenyl]quinoxalin-6-amine
N-(4-{7-[(4-methanesulfonylpyridin-3-yl)amino]quinoxalin-5-yl}phenyl)pyrrolidine-2-carboxamide
N-(3-{7-[(4-methanesulfonylpyridin-3-yl)amino]quinoxalin-5-yl}phenyl)pyrrolidine-2-carboxamide
8-(1-ethyl-1H-indol-6-yl)-N-(4-methanesulfonylpyridin-3-yl)quinoxalin-6-amine
N-(4-methanesulfonylpyridin-3-yl)-8-(1-methyl-1H-1,2,3-benzotriazol-5-yl)quinoxalin-6-amine
2-{[8-(1-methyl-1H-indol-6-yl)quinoxalin-6-yl]amino}-N-[(1-methylpyrrolidin-3-yl)methyl]benzene-1-sulfonamide
N-(4-methanesulfonylpyridin-3-yl)-8-(2-methyl-1,3-benzothiazol-5-yl)quinoxalin-6-amine
N-(4-methanesulfonylpyridin-3-yl)-8-(1-methyl-1H-1,2,3-benzotriazol-6-yl)quinoxalin-6-amine
2-{[8-(1-methyl-1H-indol-6-yl)quinoxalin-6-yl]amino}-N-(1-methylpyrrolidin-3-yl)benzene-1-sulfonamide
2-{[8-(1-methyl-1H-indol-6-yl)quinoxalin-6-yl]amino}-N-[(oxan-4-yl)methyl]benzene-1-sulfonamide
2-{[8-(1-methyl-1H-indol-6-yl)quinoxalin-6-yl]amino}-N-[(1-methyl-1H-pyrazol-4-yl)methyl]benzene-1-sulfonamide
8-(2-amino-1,3-benzothiazol-6-yl)-N-(4-methanesulfonylpyridin-3-yl)quinoxalin-6-amine
N-{4-[(dimethylamino)methyl]pyridin-3-yl}-8-(1-methyl-1H-indol-6-yl)quinoxalin-6-amine
N-{2-[(dimethylamino)methyl]phenyl}-8-(1-methyl-1H-indol-6-yl)quinoxalin-6-amine
2-{[8-(1-methyl-1H-indol-6-yl)quinoxalin-6-yl]amino}benzoic acid
3-{[8-(1-methyl-1H-indol-6-yl)quinoxalin-6-yl]amino}pyridine-4-carboxylic acid
3-{[8-(1-methyl-1H-indol-6-yl)quinoxalin-6-yl]amino}-N-(1-methylazetidin-3-yl)pyridine-4-carboxamide
N-methyl-3-{[8-(1-methyl-1H-indol-6-yl)quinoxalin-6-yl]amino}-N-(1-methylpyrrolidin-3-yl)pyridine-4-carboxamide
2-{[8-(-methyl-1H-indol-6-yl)quinoxalin-6-yl]amino}-N-(1-methylpyrrolidin-3-yl)benzamide
N-(5-{7-[(4-methanesulfonylpyridin-3-yl)amino]quinoxalin-5-yl}-1,3-benzothiazol-2-yl)pyrrolidine-2-carboxamide
N-(4-methanesulfonylpyridin-3-yl)-8-(1-propyl-1H-indol-6-yl)quinoxalin-6-amine
N-(6-{7-[(4-methanesulfonylpyridin-3-yl)amino]quinoxalin-5-yl}-1,3-benzothiazol-2-yl)pyrrolidine-2-carboxamide
N-(4-methanesulfonylpyridin-3-yl)-8-[4-(trifluoromethyl)phenyl]quinoxalin-6-amine
8-(4-amino-3-fluorophenyl)-N-(4-methanesulfonylpyridin-3-yl)quinoxalin-6-amine
N-methyl-2-{[8-(1-methyl-1H-indol-6-yl)quinoxalin-6-yl]amino}-N-[(pyrimidin-5-yl)methyl]benzene-1-sulfonamide
8-(4-fluorophenyl)-N-(4-methanesulfonylpyridin-3-yl)quinoxalin-6-amine 8-(1,4-dimethyl-1H-indol-6-yl)-N-(4-methanesulfonylpyridin-3-yl)quinoxalin-6-amine
8-(2-amino-1,3-benzothiazol-5-yl)-N-(2-methanesulfonylphenyl)quinoxalin-6-amine
N-(2-methanesulfonylphenyl)-8-(3-methyl-1-benzothiophen-5-yl)quinoxalin-6-amine
8-(3,5-diethylphenyl)-N-(4-methanesulfonylpyridin-3-yl)quinoxalin-6-amine
3-{[8-(3-methyl-1-benzothiophen-5-yl)quinoxalin-6-yl]amino}-N-[(3S)-1 methylpyrrolidin-3-yl]pyridine-4-carboxamide
3-{[8-(3-methyl-1-benzothiophen-5-yl)quinoxalin-6-yl]amino}-N-[(3R)-1 methylpyrrolidin-3-yl]pyridine-4-carboxamide
8-[2-(dimethylamino)-5-methylphenyl]-N-(4-methanesulfonylpyridin-3-yl)quinoxalin-6-amine
N-(1-methyl-1H-1,2,3-triazol-5-yl)-8-(1-methyl-1H-indol-6-yl)quinoxalin 6-amine
8-(1,5-dimethyl-1H-indol-6-yl)-N-(4-methanesulfonylpyridin-3-yl)quin-oxalin-6-amine
3-{[8-(4-fluoro-1-methyl-1H-indol-6-yl)quinoxalin-6-yl]amino}-N-(1-methylpyrrolidin-3-yl)pyridine-4-carboxamide
3-{[8-(1-methyl-1H-indol-6-yl)quinoxalin-6-yl]oxy}pyridine-4-carboxylic acid
2-{[8-(3-methyl-1-benzothiophen-5-yl)quinoxalin-6-yl]amino}-N-(1-methylpyrrolidin-3-yl)benzene-1-sulfonamide
N-(5-{7-[(4-methanesulfonylpyridin-3-yl)amino]quinoxalin-5-yl}-1-benzothiophen-2-yl)acetamide
8-[2-(dimethylamino)-1,3-benzothiazol-5-yl]-N-(4-methanesulfonylpyridin-3-yl)quinoxalin-6-amine
3-{[8-(1-methyl-1H-indol-6-yl)quinoxalin-6-yl]amino}-N-(pyrimidin-4-yl)pyridine-4-carboxamide
N-(1-acetylazetidin-3-yl)-3-{[8-(3-methyl-1-benzothiophen-5-yl)quin-oxalin-6-yl]amino}pyridine-4-carboxamide
8-(1-methyl-1H-indol-6-yl)-N-(4-{[(pyrimidin-5-yl)amino]methyl}pyridin-3-yl)quinoxalin-6-amine
2-{[8-(1-methyl-1H-indol-6-yl)quinoxalin-6-yl]amino}-N-(1-methylpiperi-din-3-yl)benzene-1-sulfonamide
2-{[8-(1-methyl-1H-indol-6-yl)quinoxalin-6-yl]amino}-N-(oxan-3-yl)-benzene-1-sulfonamide
N-(4-methanesulfonylpyridin-3-yl)-8-[3-(methylsulfanyl)phenyl]quin-oxalin-6-amine
N-(4-methanesulfonylpyridin-3-yl)-8-[3-(trifluoromethyl)-1-benzothio-phen-5-yl]quinoxalin-6-amine
8-(4-bromo-3-fluorophenyl)-N-(4-methanesulfonylpyridin-3-yl)quinoxalin-6-amine
8-(4-bromo-2-methylphenyl)-N-(4-methanesulfonylpyridin-3-yl)quin-oxalin-6-amine
N-(4-methanesulfonylpyridin-3-yl)-8-[4-(pentafluoro-λ⁶-sulfanyl)phenyl]-quinoxalin-6-amine
3-{[8-(2-amino-1,3-benzothiazol-5-yl)quinoxalin-6-yl]amino}-N-(1-methylpyrrolidin-3-yl)pyridine-4-carboxamide
3-{[8-(4-bromophenyl)quinoxalin-6-yl]amino}-N-(1-methylpyrrolidin-3-yl)-pyridine-4-carboxamide
N-(4-methanesulfonylpyridin-3-yl)-8-[2-(methylamino)-1,3-benzothiazol-5-yl]quinoxalin-6-amine
5-{7-[(4-methanesulfonylpyridin-3-yl)amino]quinoxalin-5-yl}-2,3-dihydro-1,3-benzothiazol-2-one (5-{7-[(4-methanesulfonylpyridin-3-yl)amino]quinoxalin-5-yl}-1,3-benzothiazol-2-ol)
8-(2-amino-1-benzothiophen-5-yl)-N-(4-methanesulfonylpyridin-3-yl)-quinoxaiin-6-amine
8-(1-methyl-1H-indol-6-yl)-N-{4-[(methylamino)methyl]pyridin-3-yl}-quinoxalin-6-amine
8-(3-methyl-1-benzothiophen-5-yl)-N-{4-[(methylamino)methyl]pyridin-3-yl}quinoxalin-6-amine
N-(5-bromopyrimidin-4-yl)-8-(1-methyl-1H-indol-6-yl)quinoxalin-6-amine 3-{[8-(3-methyl-1-benzothiophen-5-yl)quinoxalin-6-yl]amino}pyridine-4-carboxamide
3-{[8-(1-methyl-1H-indol-6-yl)quinoxalin-6-yl]amino}-N-(pyridin-3-yl)pyridine-4-carboxamide
8-(2-amino-1-benzothiophen-6-yl)-N-(4-methanesulfonylpyridin-3-yl)quinoxalin-6-amine
3-{[8-(1-methyl-1H-indol-6-yl)quinoxalin-6-yl]oxy}pyridine-4-carb-oxamide
3-{[8-(3-methyl-1-benzothiophen-5-yl)quinoxalin-6-yl]amino}-N-(5-oxo-pyrrolidin-3-yl)pyridine-4-carboxamide
3-{[8-(3-methyl-1-benzothiophen-5-yl)quinoxalin-6-yl]amino}-N-(2-oxopyrrolidin-3-yl)pyridine-4-carboxamide
3-{[8-(3-methyl-1-benzothiophen-5-yl)quinoxalin-6-yl]amino}-N-(1-methyl-5-oxopyrrolidin-3-yl)pyridine-4-carboxamide
3-{[8-(3-methyl-1-benzothiophen-5-yl)quinoxalin-6-yl]amino}-N-(1-methyl-2-oxopyrrolidin-3-yl)pyridine-4-carboxamide
8-(1-methyl-1H-indol-6-yl)-N-{4-[(methylamino)methyl]pyridin-3-yl}-quinoxalin-6-amine
N-methyl-3-{[8-(3-methyl-1-benzothiophen-5-yl)quinoxalin-6-yl]amino}-pyridine-4-carboxamide
3-{[8-(4-bromophenyl)quinoxalin-6-yl]amino}-N-(1-methylpyrrolidin-3-yl) pyridine-4-carboxamide
3-{[8-(2-amino-1,3-benzothiazol-5-yl)quinoxalin-6-yl]amino}-N-(1-methylpyrrolidin-3-yl)pyridine-4-carboxamide
N-(4-methanesulfonylpyridin-3-yl)-8-[4-(pentafluoro-λ⁶-sulfanyl)phenyl]-quinoxalin-6-amine
3-{[8-(1-methyl-1H-indol-6-yl)quinoxalin-6-yl]amino}-N-(pyrimidin-4-yl)pyridine-4-carboxamide
8-(1-methyl-1H-indol-6-yl)-N-(4-{[(pyrimidin-5-yl)amino]methyl}pyridin-3-yl)quinoxalin-6-amine
2-{[8-(1-methyl-1H-indol-6-yl)quinoxalin-6-yl]amino}-N-(1-methyl-piperidin-3-yl)benzene-1-sulfonamide
2-{[8-(1-methyl-1H-indol-6-yl)quinoxalin-6-yl]amino}-N-(oxan-3-yl)-benzene-1-sulfonamide
N-(4-methanesulfonylpyridin-3-yl)-8-[3-(methylsulfanyl)phenyl]quinoxalin-6-amine
8-(4-bromo-3-fluorophenyl)-N-(4-methanesulfonylpyridin-3-yl)quinoxalin-6-amine
8-(4-bromo-2-methylphenyl)-N-(4-methanesulfonylpyridin-3-yl)quinoxalin-6-amine
2-amino-N-(5-{7-[(4-methanesulfonylpyridin-3-yl)amino]quinoxalin-5-yl}-1-benzothiophen-2-yl)acetamide
N-(5-fluoro-1-methylpyrrolidin-3-yl)-3-{[8-(3-methyl-1-benzothiophen-5-yl)quinoxalin-6-yl]amino}pyridine-4-carboxamide
N-(3-fluoro-1-methylpyrrolidin-3-yl)-3-{[8-(3-methyl-1-benzothiophen-5-yl)quinoxalin-6-yl]amino}pyridine-4-carboxamide
N-(5,5-difluoro-1-methylpyrrolidin-3-yl)-3-{[8-(3-methyl-1-benzothiophen-5-yl)quinoxalin-6-yl]amino}pyridine-4-carboxamide
N-(3,3-difluoro-1-methylpyrrolidin-3-yl)-3-{[8-(3-methyl-1-benzothiophen-5-yl)quinoxalin-6-yl]amino}pyridine-4-carboxamide.

12. A pharmaceutical composition comprising at least one compound of formula (I) as defined in any one of items 1 to 11, or its derivatives, N-oxides, prodrugs, solvates, tautomers or stereoisomers thereof as well as the physiologically acceptable salts of each of the foregoing, including mixtures thereof in all ratios, as active ingredient, together with a pharmaceutically acceptable carrier.

The pharmaceutical composition according to item 12 that further comprises a second active ingredient or its derivatives, N-oxides, prodrugs, solvates, tautomers or stereoisomers thereof as well as the physiologically acceptable salts of each of the foregoing, including mixtures thereof in all ratios, wherein that second active ingredient is than a compound of formula (I) as defined in any one of items 1

Medicament comprising at least one compound of formula (I) as defined in any one of items 1 to 11, or its derivatives, N-oxides, prodrugs, solvates, tautomers or stereoisomers thereof as well as the

physiologically acceptable salts of each of the foregoing, including mixtures thereof in all ratios.

16. The compound of formula (I) as defined in any one of items 1 to 11, or its derivatives, N-oxides, prodrugs, solvates, tautomers or stereoisomers thereof as well as the physiologically acceptable salts of each of the foregoing, including mixtures thereof in all ratios.

17. Set (kit) comprising separate packs of

a) an effective amount of a compound of formula (I) as defined in any one of items 1 to 11, or its derivatives, N-oxides, prodrugs, solvates, tautomers or stereoisomers thereof as well as the physiologically acceptable salts of each of the foregoing, including mixtures thereof in all ratios; and

b) an effective amount of a further active ingredient that further active ingredient not being a compound of formula (I) as defined in any one of items 1 to 11.

Item F

PFKFB3 inhibitor for use in neuroprotection, wherein PFKFB3 is selected from those described in WO2018087021 A1 incorporated here by reference.

PFKFB3 inhibitor for use in neuroprotection, wherein PFKFB3 inhibitor is selected from the following 1. Compound of formula I

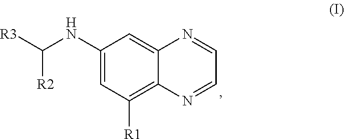

wherein R1 denotes N-methyl-indol-6-yl (1-methyl-1H-indol-6-yl), 3-methyl-1-benzofuran-5-yl, 1-methyl-1H-pyrrolo[3,2-b]pyridin-6-yl; R2 denotes 1H-pyrazol-4-yl or 1-methyl-1H-pyrazol-4-yl and R3 denotes 1H-imidazol-2-yl, 1-methyl-1H-imidazol-2-yl, 1H-imidazol-5-yl, 1-methyl-1H-imidazol-5-yl, 1H-1,2,3-triazol-5-yl, 1-methyl-1H-1,2,3-triazol-5-yl, morpholin-2-yl, morpholin-3-yl, pyridin-3-yl, pyridin-4-yl, 4H-1,2,4-triazol-3-yl, 4-methyl-4H-1,2,4-triazol-3-yl; or

R2 denotes 1H-pyrazol-3-yl or 1-methyl-1H-pyrazol-3-yl and

R3 denotes 1H-1,2,3-triazol-5-yl, 1-methyl-1H-1,2,3-triazol-5-yl, 4H-1,2,4-triazol-3-yl, 4-methyl-4H-1,2,4-triazol-3-yl; or

R2 denotes 1H-pyridazin-6-on-3-yl, 6-methoxypyridazin-3-yl and

R3 denotes pyridin-3-yl, pyridin-4-yl; or derivatives, N-oxides, prodrugs, solvates, tautomers or

stereoisomers thereof as well as the physiologically acceptable salts of each of the foregoing, including mixtures thereof in all ratios.

(j) Compound according to item 1, or derivatives, N-oxides, prodrugs, solvates, tautomers or stereoisomers thereof as well as the physiologically acceptable salts of each of the foregoing, including mixtures thereof in all ratios, wherein

R1 denotes N-methyl-indol-6-yl (1-methyl-1H-indol-6-yl), 3-methyl-1 benzofuran-5-yl;

R2 denotes 1-methyl-1H-pyrazol-4-yl and

R3 denotes 1-methyl-1H-imidazol-2-yl, 1-methyl-1H-imidazol-5-yl, 1 methyl-1H-1,2,3-triazol-5-yl, morpholin-2-yl, pyridine-3-yl, 4-methyl-4H-1,2,4-triazol-3-yl; or R2 denotes 1-methyl-1H-pyrazol-3-yl and

R3 denotes 1-methyl-1H-1,2,3-triazol-5-yl, 4-methyl-4H-1,2,4-triazol 3-yl;

or R2 denotes 1H-pyridazin-6-on-3-yl, 6-methoxypyridazin-3-yl and R3 denotes pyridine-3-yl.

3. Compound according to any of items 1 or 2, or derivatives, N-oxides, prodrugs, solvates, tautomers or stereoisomers thereof as well as the physiologically acceptable salts of each of the foregoing, including mixtures thereof in all ratios, wherein R1 denotes N-methyl-indol-6-yl (1-methyl-1H-indol-6-yl), 3-methyl-1-benzofuran-5-yl;

R2 denotes 1-methyl-1H-pyrazol-4-yl and

R3 denotes 1-methyl-1H-imidazol-2-yl, 1-methyl-1H-imidazol-5-yl, 1-methyl-1H-1,2,3-triazol-5-yl, morpholin-2-yl, pyridine-3-yl, 4-methyl-4H-1,2,4-triazol-3-yl.

4. Compound according to any of items 1 to 3, or derivatives, N-oxides, prodrugs, solvates, tautomers or stereoisomers thereof as well as the physiologically acceptable salts of each of the foregoing, including mixtures thereof in all ratios, wherein R1 denotes N-methyl-indol-6-yl (1-methyl-1H-indol-6-yl), 3-methyl-1 benzofuran-5-yl; R2 denotes 1-methyl-1H-pyrazol-4-yl and R3 denotes 1-methyl-1H-imidazol-2-yl, 1-methyl-1H-imidazol-5-yl, 1 methyl-1H-1,2,3-triazol-5-yl, morpholin-2-yl, pyridin-3-yl, 4-methyl-4H-1,2,4-triazol-3-yl.

5. Compound according to any of items 1 to 4, or derivatives, N-oxides, prodrugs, solvates, tautomers or stereoisomers thereof as well as the physiologically acceptable salts of each of the foregoing, including mixtures thereof in all ratios, wherein R1 denotes N-methyl-indol-6-yl (1-methyl-1H-indol-6-yl), 3-methyl-1 benzofuran-5-yl; R2 denotes 1-methyl-1H-pyrazol-4-yl and R3 denotes 1-methyl-1H-imidazol-2-yl, 1-methyl-1H-imidazol-5-yl, 1 methyl-1H-1,2,3-triazol-5-yl.

6. Compound according to any of items 1 or 2, or derivatives, N-oxides, prodrugs, solvates, tautomers or stereoisomers thereof as well as the physiologically acceptable salts of each of the foregoing, including mixtures thereof in all ratios, wherein R1 denotes N-methyl-indol-6-yl; R2 denotes 1-methyl-1H-pyrazol-3-yl and R3 denotes 1-methyl-1H-1,2,3-triazol-5-yl, 4-methyl-4H-1,2,4-triazol 3-yl.

7. Compound according to any of items 1 or 2, or derivatives, N-oxides, prodrugs, solvates, tautomers or stereoisomers thereof as well as the physiologically acceptable salts of each of the foregoing, including mixtures thereof in all ratios, wherein R1 denotes N-methyl-indol-6-yl; R2 denotes 1H-pyridazin-6-on-3-yl or 6-methoxypyridazin-3-yl and R3 denotes pyridin-3-yl.

8. Compound, or the N-oxides and/or physiologically acceptable salts thereof, selected from the group consisting of:
6-[{[8-(1-methyl-1H-indol-6-yl)quinoxalin-6-yl]amino}(pyridin-3-yl)methyl]-2,3-dihydropyridazin-3-one
(6-{[8-(1-Methyl-1H-indol-6-yl)-quinoxalin-6-ylamino]-pyridin-3-yl-methyl}-2H-pyridazin-3-one) 6-[(S)-{[8-(1-methyl-1H-indol-6-yl)quinoxalin-6-yl]amino}(pyridin-3-yl)methyl]-2,3-dihydropyridazin-3-one
6-[(R)-{[8-(1-methyl-1H-indol-6-yl)quinoxalin-6-yl]amino}(pyridin-3-yl)methyl]-2,3-dihydropyridazin-3-one
N-[(1-methyl-1H-imidazol-2-yl)(1-methyl-1H-pyrazol-4-yl)methyl]-8-(1-methyl-1H-indol-6-yl)quinoxalin-6-amine
N-[(S)-(1-methyl-1H-imidazol-2-yl)(1-methyl-1H-pyrazol-4-yl)methyl]-8 (1-methyl-1H-indol-6-yl)quinoxalin-6-amine
N-[(R)-(1-methyl-1H-imidazol-2-yl)(1-methyl-1H-pyrazol-4-yl)methyl]-8 (1-methyl-1H-indol-6-yl)quinoxalin-6-amine
N-[(6-methoxypyridazin-3-yl)(pyridin-3-yl)methyl]-8-(1-methyl-1H-indol 6-yl)quinoxalin-6-amine
N-[(S)-(6-methoxypyridazin-3-yl)(pyridin-3-yl)methyl]-8-(1-methyl-1H-indol-6-yl)quinoxalin-6-amine
N-[(R)-(6-methoxypyridazin-3-yl)(pyridin-3-yl)methyl]-8-(1-methyl-1H-indol-6-yl)quinoxalin-6-amine
N-[(1-methyl-1H-1,2,3-triazol-5-yl)(1-methyl-1H-pyrazol-4-yl)methyl]-8-(1-methyl-1H-indol-6-yl)quinoxalin-6-amine ([8-(1-Methyl-1H-indol-6-yl)-quinoxalin-6-yl]-[(1-methyl-1H-pyrazol-4-yl)-(3-methyl-3H-[1,2,3]triazol-4-yl)-methyl]-amine)
N-[(S)-(1-methyl-1H-1,2,3-triazol-5-yl)(1-methyl-1H-pyrazol-4-yl)methyl] 8-(1-methyl-1H-indol-6-yl)quinoxalin-6-amine
N-[(R)-(1-methyl-1H-1,2,3-triazol-5-yl)(1-methyl-1H-pyrazol-4-yl)methyl]8-(1-methyl-1H-indol-6-yl)quinoxalin-6-amine
N-(1-methyl-1H-imidazol-5-yl)(1-methyl-1H-pyrazol-4-yl)methyl]-8-(1-methyl-1H-indol-6-yl)quinoxalin-6-amine ([(3-Methyl-3H-imidazol-4-yl)-(1-methyl-1H-pyrazol-4-yl)-methyl]-[8-(1-methyl-1H-indol-6-yl)-quinoxalin-6-yl]-amine)
N-[(S)-(1-methyl-1H-imidazol-5-yl)(1-methyl-1H-pyrazol-4-yl)methyl]-8-(1-methyl-1H-indol-6-yl)quinoxalin-6-amine
N-[(R)-(1-methyl-1H-imidazol-5-yl)(1-methyl-1H-pyrazol-4-yl)methyl]-8-(1-methyl-1H-indol-6-yl)quinoxalin-6-amine
N-[(1-methyl-1H-1,2,3-triazol-5-yl)(1-methyl-1H-pyrazol-3-yl)methyl]-8-(1-methyl-1H-indol-6-yl)quinoxalin-6-amine ([8-(1-Methyl-1H-indol-6-yl)-quinoxalin-6-yl]-[(1-methyl-1H-pyrazol-3-yl)-(3-methyl-3H-[1,2,3]triazol-4-yl)-methyl]-amine)
N-[(S)-(1-methyl-1H-1,2,3-triazol-5-yl)(1-methyl-1H-pyrazol-3-yl)methyl] 8-(1-methyl-1H-indol-6-yl)quinoxalin-6-amine
N-[(R)-(1-methyl-1H-1,2,3-triazol-5-yl)(1-methyl-1H-pyrazol-3-yl)methyl]8-(1-methyl-1H-indol-6-yl)quinoxalin-6-amine
8-(1-methyl-1H-indol-6-yl)-N-[(1-methyl-1H-pyrazol-4-yl)(morpholin-2-yl)methyl]quinoxalin-6-amine
8-(1-methyl-1H-indol-6-yl)-N-[(S)-(1-methyl-1H-pyrazol-4-yl)(morpholin-2-yl)methyl]]quinoxalin-6-amine
8-(1-methyl-1H-indol-6-yl)-N-[(R)-(1-methyl-1H-pyrazol-4-yl)(morpholin-2-yl)methyl]]quinoxalin-6-amine
8-(1-methyl-1H-indol-6-yl)-N-[(1-methyl-1H-pyrazol-4-yl)(pyridin-3-yl)methyl]quinoxalin-6-amine
8-(1-methyl-1H-indol-6-yl)-N-[(R)-(1-methyl-1H-pyrazol-4-yl)(pyridin-3-yl)methyl]quinoxalin-6-amine
8-(1-methyl-1H-indol-6-yl)-N-[(S)-(1-methyl-1H-pyrazol-4-yl)(pyridin-3-yl)methyl]quinoxalin-6-amine
8-(1-methyl-1H-indol-6-yl)-N-[(1-methyl-1H-pyrazol-4-yl)(4-methyl-4H-1,2,4-triazol-3-yl)methyl]quinoxalin-6-amine
8-(1-methyl-1H-indol-6-yl)-N-[(S)-(1-methyl-1H-pyrazol-4-yl)(4-methyl-4H-1,2,4-triazol-3-yl)methyl]quinoxalin-6-amine
8-(1-methyl-1H-indol-6-yl)-N-[(S)-(1-methyl-1H-pyrazol-4-yl)(4-methyl-4H-1,2,4-triazol-3-yl)methyl]quinoxalin-6-amine
8-(1-methyl-1H-indol-6-yl)-N-[(1-methyl-1H-pyrazol-3-yl)(4-methyl-4H-1,2,4-triazol-3-yl)methyl]quinoxalin-6-amine
8-(1-methyl-1H-indol-6-yl)-N-[(S)-(1-methyl-1H-pyrazol-3-yl)(4-methyl-4H-1,2,4-triazol-3-yl)methyl]quinoxalin-6-amine
8-(1-methyl-1H-indol-6-yl)-N-[(S)-(1-methyl-1H-pyrazol-3-yl)(4-methyl-4H-1,2,4-triazol-3-yl)methyl]quinoxalin-6-amine
8-(3-methyl-1-benzofuran-5-yl)-N-[(1-methyl-1H-1,2,3-triazol-5-yl)(1-methyl-1H-pyrazol-4-yl)methyl]quinoxalin-6-amine ([8-(3-Methyl-benzofuran-5-yl)-quinoxalin-6-yl]-[(1-methyl-1H-pyrazol-4-yl)-(3-methyl-3H-[1,2,3]triazol-4-yl)-methyl]-amine)
8-(3-methyl-1-benzofuran-5-yl)-N-[(S)-(1-methyl-1H-1,2,3-triazol-5-yl)(1 methyl-1H-pyrazol-4-yl)methyl]quinoxalin-6-amine
8-(3-methyl-1-benzofuran-5-yl)-N-[(S)-(1-methyl-1H-1,2,3-triazol-5-yl)(1 methyl-1H-pyrazol-4-yl)methyl]quinoxalin-6-amine
N-[(6-methoxypyridin-3-yl)(morpholin-2-yl)methyl]-8-(1-methyl-1H-indol6-yl)quinoxalin-6-amine
N-[(S)-(6-methoxypyridin-3-yl)(morpholin-2-yl)methyl]-8-(1-methyl-1H-indol-6-yl)quinoxalin-6-amine
N-[(R)-(6-methoxypyridin-3-yl)(morpholin-2-yl)methyl]-8-(1-methyl-1H-indol-6-yl)quinoxalin-6-amine
[8-(1-Methyl-1H-indol-6-yl)-quinoxalin-6-yl]-(2-methyl-1-pyridin-3-yl-propyl)-amine
[8-(1-Methyl-1H-indol-6-yl)-quinoxalin-6-yl]-((R)-2-methyl-1-pyridin-3-yl-propyl)-amine
[8-(1-Methyl-1H-indol-6-yl)-quinoxalin-6-yl]-((S)-2-methyl-1-pyridin-3-yl-propyl)-amine
N-[2-(1-Methyl-1H-1,2,3-triazol-5-yl)propan-2-yl]-8-(1-methyl-1H-indol-6-yl)quinoxalin-6-amine
8-(1-Methyl-1H-indol-6-yl)-N-[2-(morpholin-2-yl)propan-2-yl]quinoxalin-6-amine
[(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine
[(S)-(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine
[(R)-(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine.

9. Compound according to item 8, or the N-oxides and/or physiologically acceptable salts thereof, selected from the group consisting of:
6-[{[8-(1-methyl-1H-indol-6-yl)quinoxalin-6-yl]amino}(pyridin-3-yl)methyl]-2,3-dihydropyridazin-3-one
6-[(S)-{[8-(1-methyl-1H-indol-6-yl)quinoxalin-6-yl]amino}(pyridin-3-yl)methyl]-2,3-dihydropyridazin-3-one
6-[(R)-{[8-(1-methyl-1H-indol-6-yl)quinoxalin-6-yl]amino}(pyridin-3-yl)methyl]-2,3-dihydropyridazin-3-one
N-[(S)-(1-methyl-1H-imidazol-2-yl)(1-methyl-1H-pyrazol-4-yl)methyl]-8-(1-methyl-1H-indol-6-yl)quinoxalin-6-amine
N-[(R)-(1-methyl-1H-imidazol-2-yl)(1-methyl-1H-pyrazol-4-yl)methyl]-8-(1-methyl-1H-indol-6-yl)quinoxalin-6-amine
N-[(S)-(6-methoxypyridazin-3-yl)(pyridin-3-yl)methyl]-8-(1-methyl-1H-indol-6-yl)quinoxalin-6-amine
N-[(R)-(6-methoxypyridazin-3-yl)(pyridin-3-yl)methyl]-8-(1-methyl-1H-indol-6-yl)quinoxalin-6-amine
N-[(S)-(1-methyl-1H-1,2,3-triazol-5-yl)(1-methyl-1H-pyrazol-4-yl)methyl] 8-(1-methyl-1H-indol-6-yl)quinoxalin-6-amine
N-[(R)-(1-methyl-1H-1,2,3-triazol-5-yl)(1-methyl-1H-pyrazol-4-yl)methyl] 8-(1-methyl-1H-indol-6-yl)quinoxalin-6-amine
N-[(S)-(1-methyl-1H-imidazol-5-yl)(1-methyl-1H-pyrazol-4-yl)methyl]-8-(1-methyl-1H-indol-6-yl)quinoxalin-6-amine
N-[(R)-(1-methyl-1H-imidazol-5-yl)(1-methyl-1H-pyrazol-4-yl)methyl]-8-(1-methyl-1H-indol-6-yl)quinoxalin-6-amine
8-(1-methyl-1H-indol-6-yl)-N-[(R)-(1-methyl-1H-pyrazol-4-yl)(pyridin-3-yl)methyl]quinoxalin-6-amine
8-(1-methyl-1H-indol-6-yl)-N-[(S)-(1-methyl-1H-pyrazol-4-yl)(pyridin-3-yl)methyl]quinoxalin-6-amine
8-(3-methyl-1-benzofuran-5-yl)-N-[(S)-(1-methyl-1H-1,2,3-triazol-5-yl)(1 methyl-1H-pyrazol-4-yl)methyl]quinoxalin-6-amine
8-(3-methyl-1-benzofuran-5-yl)-N-[(S)-(1-methyl-1H-1,2,3-triazol-5-yl)(1 methyl-1H-pyrazol-4-yl)methyl]quinoxalin-6-amine
N-[(S)-(1-methyl-1H-1,2,3-triazol-5-yl)(1-methyl-1H-pyrazol-3-yl)methyl]8-(1-methyl-1H-indol-6-yl)quinoxalin-6-amine
N-[(R)-(1-methyl-1H-1,2,3-triazol-5-yl)(1-methyl-1H-pyrazol-3-yl)methyl] 8-(1-methyl-1H-indol-6-yl)quinoxalin-6-amine.

10. A pharmaceutical composition comprising at least one compound according to any one of items 1 to 9, or its derivatives, N-oxides, prodrugs, solvates, tautomers or stereoisomers thereof as well as the physiologically acceptable salts of each of the foregoing, including mixtures thereof in all ratios, as active ingredient, together with a pharmaceutically acceptable carrier.

11. The pharmaceutical composition according to item 10 that further comprises a second active ingredient or its derivatives, N-oxides, prodrugs, solvates, tautomers or stereoisomers thereof as well as the physiologically acceptable salts of each of the foregoing, including mixtures thereof in all ratios, wherein that second active ingredient is other than a compound according to any one of items 1 to 9.

12. Medicament comprising at least one compound according to any one of items 1 to 9, or its derivatives, N-oxides, prodrugs, solvates, tautomers or stereoisomers thereof as well as the physiologically acceptable salts of each of the foregoing, including mixtures thereof in all ratios.

Item G

PFKFB3 inhibitor for use in neuroprotection, wherein PFKFB3 is selected from those described in applications WO2011161201 A1, EP2794009B1 and publication 0.1038/s41467-018-06287-x (https://www.nature.com/articles/s41467-018-06287-x 10), incorporated here by reference.

PFKFB3 inhibitor for use in neuroprotection, wherein PFKFB3 inhibitor is selected from the following

1. PFKFB3 inhibitor for use in neuroprotection, wherein a PFKFB3 inhibitor is of formula (I)

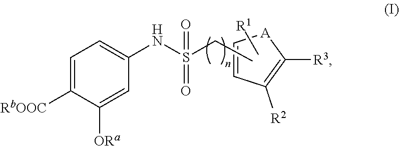

wherein n is 0 or 1; A is —CR⁴═CR⁴—;

R¹ is selected from H; halogen; C1-C6 alkyl, optionally substituted with at least one halogen; and C1-C6 alkoxy, optionally substituted with at least one halogen;

R² is selected from carbocyclyl-C0-C3 alkyl and heterocyclyl-C0-C3 alkyl; wherein any alkyl is optionally substituted with at least one halogen; any carbocyclyl or heterocyclyl is 5- or 6-membered monocyclyl or 9- or 10-membered bicyclyl; and any carbocyclyl or heterocyclyl is optionally substituted with at least one R⁵;

R³ is selected from H; halogen; C1-C6 alkyl; C1-C6 alkoxy; C1-C6 alkylcarbonylamino; hydroxy-C0-C6 alkyl, C1-C6 alkylcarbonyl; C1-C6 alkoxycarbonyl; and cyano; wherein any alkyl is optionally substituted with at least one halogen;

or R² and R³ form, together with the carbon atoms to which they are attached, a 5- or 6-membered carbocyclic or heterocyclic ring, which ring is optionally substituted with at least one R⁵;

each R⁴ is independently selected from H, halogen, monocyclic C3-C6 carbocyclyl and C1-C6 alkyl, wherein any alkyl is optionally substituted with at least one halogen;

each R⁵ is independently selected from halogen; C1-C6 alkyl; C1-C6 alkoxy; phenoxy; amino; cyano; nitro; secondary or tertiary C1-C6 alkylamino; 5- or 6-membered cyclic amino, optionally containing at least one further heteroatom in the ring; C1-C6 alkylcarbonylamino; carbamoyl; secondary or tertiary C1-C6 alkylamido; 5- or 6-membered cyclic aminocarbonyl; C1-C6 alkoxycarbonylamino; hydroxy-C0-C6 alkyl; C1-C6-alkylthio; carboxy-C0-C6-alkyl; C1-C6 alkoxycarbonyl; C1-C6 alkylcarbonyl; C1-C6-alkylsulfonyl; and C1-C6 alkylsulfonylamino; wherein any alkyl is optionally substituted with at least one halogen;

R^{a} is selected from H and C1-C6 alkylcarbonyl;

R^{b} is selected from H, C1-C6 alkyl, C1-C6 alkyl substituted with at least one R⁶; carbocyclyl-C0-C5 alkyl; and heterocyclyl-C0-C5 alkyl; wherein any carbocyclyl and heterocyclyl is 5- or 6-membered and is optionally substituted with at least one R⁷ and optionally comprises at least one oxo group in the ring;

provided that R^{a} and R^{b} are not both H;

each R⁶ is independently selected from hydroxy; C1-C6 alkoxy; hydroxy-C1-C6 alkoxy; C1-C6 alkylcarbonyloxy; C1-C6 alkoxycarbonyloxy; 5- or 6-membered carbocyclylcarbonyl or heterocyclylcarbonyl; amino; secondary or tertiary C1-C6 alkylamino; secondary or tertiary hydroxy-C1-C6 alkylamino; 5- or 6-membered cyclic amino optionally containing at least one further heteroatom in the ring and wherein the ring is optionally substituted with at least one C1-C6 alkyl; C1-C6 alkylcarbonylamino; C1-C6 alkoxycarbonylamino; (C1-C6 alkoxycarbonyl)(C1-C6 alkyl)amino; (C1-C6 alkoxycarbonyl)(5- or 6-membered carbocyclyl or heterocyclyl)amino; (C1-C6 alkylcarbonyl)(C1-C6 alkyl)amino; carbamoyl; secondary or tertiary C1-C6 alkylamido wherein any alkyl is optionally substituted by OH or CONH₂; 5- or 6-membered carbocyclyl- or heterocyclylcarbamoyl; 5- or 6-membered cyclic aminocarbonyl, optionally containing at least one further heteroatom in the ring, and wherein the ring is optionally substituted with at least one C1-C6 alkyl; 5-or-6-membered carbocyclylamino or heterocyclylamino; and 5-or-6-membered carbocyclyloxy or heterocyclyloxy; wherein any alkyl is optionally substituted with at least one halogen and any 5- or 6-membered carbocyclyl or heterocyclyl is optionally substituted with at least one R⁸;

each R⁷ and R⁸ is independently selected from C1-C6 alkyl; hydroxy-C0-C3 alkyl; C1-C6 alkoxy-C0-C3 alkyl; C1-C6 alkoxycarbonyl; carbocyclyl-C0-C4 alkyl; heterocyclyl-C0-C4 alkyl; C1-C6 alkylsulfinyl; amino; nitro; C1-C6 secondary or tertiary amino; halogen; carbamoyl; secondary or tertiary C1-C6 alkylamido-C0-C3 alkyl; C1-C6 alkylcarbonylamino; and 5- or 6-membered cyclic amino, optionally containing at least one further heteroatom in the ring and wherein the ring is optionally substituted with at least one C1-C6 alkyl; wherein any alkyl is optionally substituted with at least one halogen; wherein any carbocyclyl and heterocyclyl is 5- or 6-membered; or a pharmaceutically acceptable salt thereof.

11. The PFKFB3 inhibitor for use in neuroprotection of item 1, wherein R¹ is selected from H and C1-C3 alkyl.

12. The PFKFB3 inhibitor for use in neuroprotection of item 1, wherein each R⁴ is H.

13. The PFKFB3 inhibitor for use in neuroprotection of item 1, wherein R³ is selected from H; halogen; and C1-C6 alkyl, wherein any alkyl is optionally substituted with at least one halogen.

14. The PFKFB3 inhibitor for use in neuroprotection of item 1, wherein n is 0.

15. The PFKFB3 inhibitor for use in neuroprotection of item 1, wherein R^{a} is H.

16. The PFKFB3 inhibitor for use in neuroprotection of item 1, wherein the PFKFB3 inhibitor for use in neuroprotection is of formula (ICa)

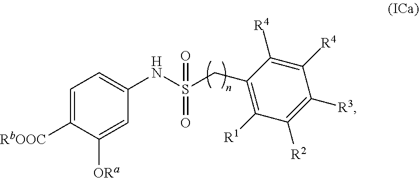

wherein R1, R4, Ra, Rb and n are as defined in item 1, R2 is phenyl substituted with at least one R5, and R3 is H.

17. The PFKFB3 inhibitor for use in neuroprotection of item 1, wherein R2 is phenyl substituted with 1 or 2 moieties R5; and each R5 is independently selected from hydroxy, C1-C3 alkoxy and halogen.

18. The PFKFB3 inhibitor for use in neuroprotection of item 1, wherein R2 is 5-fluoro-2-hydroxyphenyl.

19. The PFKFB3 inhibitor for use in neuroprotection of item 1, wherein Rb is C2-C4 alkyl, substituted by 1 or 2 moieties selected from methoxy and ethoxy.

20. The PFKFB3 inhibitor for use in neuroprotection of item 1, wherein Rb is hydroxy-C2-C4 alkyl.

21. The PFKFB3 inhibitor for use in neuroprotection of item 1, wherein Rb is tetrahydrofuryl-C1-C0 alkyl.

22. The PFKFB3 inhibitor for use in neuroprotection of item 1, wherein the PFKFB3 inhibitor for use in neuroprotection is selected from
methyl 2-hydroxy-4-{[(4-methyl-1-naphthyl)sulfonyl]amino}benzoate,
methyl 2-hydroxy-4-[(1-naphthylsulfonyl)amino]benzoate,
methyl 4-{[(4-fluoro-1-naphthyl)sulfonyl]amino}-2-hydroxybenzoate,
methyl 4-[(2,1,3-benzothiadiazol-4-ylsulfonyl)amino]-2-hydroxybenzoate,
methyl 2-hydroxy-4-[(naphthalen-2-ylsulfonyl)amino]benzoate,
methyl 4-({[5-(dimethylamino)naphthalen-1-yl]sulfonyl}amino)-2-hydroxybenzoate,
methyl 2-hydroxy-4-{[(2′-hydroxybiphenyl-3-yl)sulfonyl]amino}benzoate,
methyl 4-{[(3′-chlorobiphenyl-3-yl)sulfonyl]amino}-2-hydroxybenzoate,
methyl 4-[(biphenyl-3-ylsulfonyl)amino]-2-hydroxybenzoate,
methyl 2-hydroxy-4-{[(3-pyridin-3-ylphenyl)sulfonyl]amino}benzoate,
methyl 2-hydroxy-4-{[(3-pyridin-4-ylphenyl)sulfonyl]amino}benzoate,
methyl 4-({[3-(1-benzofuran-2-yl)phenyl]sulfonyl}amino)-2-hydroxybenzoate,
methyl 2-hydroxy-4-{[(3-quinolin-6-ylphenyl)sulfonyl]amino}benzoate,
methyl 4-{[(3′-aminobiphenyl-3-yl)sulfonyl]amino}-2-hydroxybenzoate,
methyl 4-{[(3′-acetamidobiphenyl-3-yl)sulfonyl]amino}-2-hydroxybenzoate,
methyl 2-hydroxy-4-{[(2′-nitrobiphenyl-3-yl)sulfonyl]amino}benzoate,
methyl 4-({[3-(5-acetyl-2-thienyl)phenyl]sulfonyl}amino)-2-hydroxybenzoate,
methyl 2-hydroxy-4-({[2′-(hydroxymethyl)biphenyl-3-yl]sulfonyl}amino)benzoate,
methyl 4-{[(3′-cyanobiphenyl-3-yl)sulfonyl]amino}-2-hydroxybenzoate,
methyl 2-hydroxy-4-({[4′-(methylsulfanyl)biphenyl-3-yl]sulfonyl}amino)benzoate,
methyl 2-hydroxy-4-({[4′-(trifluoromethoxy)biphenyl-3-yl]sulfonyl}amino)benzoate,
methyl 2-hydroxy-4-({[4′-(trifluoromethyl)biphenyl-3-yl]sulfonyl}amino)benzoate,
methyl 4-({[4′-(dimethylcarbamoyl)biphenyl-3-yl]sulfonyl}amino)-2-hydroxybenzoate,
methyl 4-{[(4′-carbamoylbiphenyl-3-yl)sulfonyl]amino}-2-hydroxybenzoate,
methyl 2-hydroxy-4-({[3′-(methylsulfonyl)biphenyl-3-yl]sulfonyl}amino)benzoate,
methyl 4-{[(3′-carbamoylbiphenyl-3-yl)sulfonyl]amino}-2-hydroxybenzoate,
methyl 2-hydroxy-4-({[5-(trifluoromethyl)biphenyl-3-yl]sulfonyl}amino)benzoate,
methyl 4-({[2′,5′-difluoro-5-(trifluoromethyl)biphenyl-3-yl]sulfonyl}amino)-2-hydroxybenzoate,
methyl 4-({[3-(2,3-dihydro-1-benzofuran-5-yl)-5-(trifluoromethyl)phenyl]sulfonyl}amino)-2-hydroxybenzoate,
methyl 2-hydroxy-4-({[2′-hydroxy-5-(trifluoromethyl)biphenyl-3-yl]sulfonyl}amino)benzoate,
methyl 4-({[3-(2,3-dihydro-1-benzofuran-5-yl)benzyl]sulfonyl}amino)-2-hydroxybenzoate,
methyl 4-{[(3′-ethoxybiphenyl-3-yl)sulfonyl]amino}-2-hydroxybenzoate,
1-methylethyl 3′-{[3-hydroxy-4-(methoxycarbonyl)phenyl]sulfamoyl}biphenyl-3-carboxylate,
methyl 4-{[(5′-fluoro-2′-hydroxybiphenyl-3-yl)sulfonyl]amino}-2-hydroxybenzoate,
benzyl 2-acetoxy-4-[(1-naphthylsulfonyl)amino]benzoate,
2-acetoxy-4-[(1-naphthylsulfonyl)amino]benzoic acid,
methyl 2-hydroxy-4-({[3-(piperidin-1-yl)phenyl]sulfonyl}amino)benzoate,
4-(dimethylamino)butyl 2-hydroxy-4-({[3-(2-methyl-1,3-thiazol-4-yl)phenyl]sulfonyl}amino)benzoate,
methyl 4-({[5′-fluoro-2′-hydroxy-5-(trifluoromethyl)biphenyl-3-yl]sulfonyl}amino)-2-hydroxybenzoate,
methyl 4-{[(2′,5′-difluorobiphenyl-3-yl)sulfonyl]amino}-2-hydroxybenzoate,
methyl 4-({[3-(2,3-dihydro-1-benzofuran-5-yl)phenyl]sulfonyl}amino)-2-hydroxybenzoate,
3-morpholin-4-ylpropyl 2-hydroxy-4-{[(2′-hydroxybiphenyl-3-yl)sulfonyl]amino}benzoate,
3-morpholin-4-ylpropyl 4-{[(5′-fluoro-2′-hydroxybiphenyl-3-yl)sulfonyl]amino}-2-hydroxybenzoate,
4-morpholin-4-ylbutyl 4-{[(5′-fluoro-2′-hydroxybiphenyl-3-yl)sulfonyl]amino}-2-hydroxybenzoate,
3-morpholin-4-ylpropyl 4-{[(2′,5′-difluorobiphenyl-3-yl)sulfonyl]amino}-2-hydroxybenzoate,
2-methoxyethyl 4-{[(2′,5′-difluorobiphenyl-3-yl)sulfonyl]amino}-2-hydroxybenzoate,
4-morpholin-4-ylbutyl 4-{[(2′,5′-difluorobiphenyl-3-yl)sulfonyl]amino}-2-hydroxybenzoate,
3-morpholin-4-ylpropyl 4-({[3-(2,3-dihydro-1-benzofuran-5-yl)phenyl]sulfonyl}amino)-2-hydroxybenzoate,
2-methoxyethyl 4-({[3-(2,3-dihydro-1-benzofuran-5-yl)phenyl]sulfonyl}amino)-2-hydroxybenzoate,
4-morpholin-4-ylbutyl 4-({[3-(2,3-dihydro-1-benzofuran-5-yl)phenyl]sulfonyl}amino)-2-hydroxybenzoate,
2-methoxy-1-methylethyl 4-{[(5′-fluoro-2′-hydroxybiphenyl-3-yl)sulfonyl]amino}-2-hydroxybenzoate,
tetrahydrofuran-3-yl 4-{[(5′-fluoro-2′-hydroxybiphenyl-3-yl)sulfonyl]amino}-2-hydroxybenzoate,
1-(methoxymethyl)propyl 4-{[(5′-fluoro-2′-hydroxybiphenyl-3-yl)sulfonyl]amino}-2-hydroxybenzoate,
2-ethoxy-1-(ethoxymethyl)ethyl 4-{[(5′-fluoro-2′-hydroxybiphenyl-3-yl)sulfonyl]amino}-2-hydroxybenzoate,
2-methoxybutyl 4-{[(5′-fluoro-2′-hydroxybiphenyl-3-yl)sulfonyl]amino}-2-hydroxybenzoate,
2-hydroxyethyl 4-{[(5′-fluoro-2′-hydroxybiphenyl-3-yl)sulfonyl]amino}-2-hydroxybenzoate,
3-hydroxypropyl 4-{[(5′-fluoro-2′-hydroxybiphenyl-3-yl)sulfonyl]amino}-2-hydroxybenzoate,
2-methoxyethyl 4-{[(5′-fluoro-2′-hydroxybiphenyl-3-yl)sulfonyl]amino}-2-hydroxybenzoate,
2-phenoxyethyl 4-{[(5′-fluoro-2′-hydroxybiphenyl-3-yl)sulfonyl]amino}-2-hydroxybenzoate,
3-(2,6-dimethylmorpholin-4-yl)propyl 4-{[(5′-fluoro-2′-hydroxybiphenyl-3-yl)sulfonyl]amino}-2-hydroxybenzoate,
3-(pyridin-3-ylamino)propyl 4-{[(5′-fluoro-2′-hydroxybiphenyl-3-yl)sulfonyl]amino}-2-hydroxybenzoate,
3-[(1-methyl-1H-pyrazol-5-yl)amino]propyl 4-{[(5′-fluoro-2′-hydroxybiphenyl-3-yl)sulfonyl]amino}-2-hydroxybenzoate,
3-[(5-methylisoxazol-3-yl)amino]propyl 4-{[(5′-fluoro-2′-hydroxybiphenyl-3-yl)sulfonyl]amino}-2-hydroxybenzoate,

or a pharmaceutically acceptable salt thereof.

23. 4-{[(5′-Fluoro-2′-hydroxybiphenyl-3-yl)sulfonyl] amino}-2-hydroxybenzoic acid or a pharmaceutically acceptable salt thereof for use in neuroprotection.

Item H

PFKFB3 inhibitor for use in neuroprotection, wherein PFKFB3 inhibitor is selected from of any of preceding items, wherein PFKFB3 inhibitor is selected from the compounds marked anywhere in this application as “not”. For example, if there was wording that “provided that the compound is not:

methyl 6-(4′-cyano-[1,-biphenyl]-4-ylsulfonamido)picolinate” for the purpose of this embodiment it means that PFKFB3 inhibitor is methyl 6-(4′-cyano-[1,-biphenyl]-4-ylsulfonamido)picolinate.

1545. PFKFB3 inhibitor for use in neuroprotection, wherein PFKFB3 inhibitor is selected from the Table 5.

1546. A method of inhibiting an isozyme of 6-phosphofructo-2-kinase/fructose-2,6-bisphosphatase (PFKFB) in a mammal; said method comprising administering to the mammal an effective amount of a compound selected from any one of the items 1 to 199.

1547. A method for the treatment of a disorder associated with modulation of F-2,6-P2 levels in a mammal, the method by administering, to a mammal having such disorder, a compound selected from any one of items 1 to 199 or a pharmaceutically acceptable salt thereof.

1548. A method for the treatment of a disorder associated with modulation of F-2,6-P2 levels in a mammal, the method by administering, to a mammal having such disorder, a compound selected from any one of items 1 to 199 or a pharmaceutically acceptable salt thereof, wherein disorder is selected from

cancer, inflammation or an inflammatory disorder.

1549. The method of cancer treatment, wherein the compound selected from any one of items 1 to 199 or a pharmaceutically acceptable salt thereof is administered in combination with a treatment modality inducing DNA damage in cancer cells of said mammal.

1550. The method of item 1549, wherein the treatment modality inducing DNA damage to cancer cells in said mammal comprises a radiotherapeutic treatment.

1551. The method of item 1549, wherein the treatment modality inducing DNA damage to cancer cells in said mammal comprises a chemotherapeutic treatment.

1552. The method of item 1549, wherein the treatment modality inducing DNA damage to cancer cells in said mammal comprises a radiotherapeutic and a chemotherapeutic treatment.

1553. The method of item 1549, wherein the cancer is selected from cancer of the brain, lung, liver, spleen, kidney, lymph node, small intestine, pancreas, blood cells, colon, stomach, breast, endometrium, prostate, testicle, ovary, skin, head and neck, esophagus, bone marrow, and blood.

1554. The method of item 1549, wherein the cancer is selected from breast cancer, lung cancer, prostate cancer, colorectal cancer, pancreatic cancer, haematological cancer and melanoma.

1555. The method of item 1549, wherein the cancer is selected from pancreas cancer, prostate cancer and breast cancer.

1556. The method of claim 1549, wherein the cancer is selected from cancer of the brain, lung, liver, spleen, kidney, lymph node, small intestine, pancreas, blood cells, colon, stomach, breast, endometrium, prostate, testicle, ovary, skin, head and neck, esophagus, bone marrow, and blood.

1557. A compound for use for the treatment of a disorder associated with modulation of F-2,6-P2 levels in a mammal, by administering, to a mammal having such disorder, a compound selected from any one of items 1 to 199 or a pharmaceutically acceptable salt thereof.

1558. The compound for use for cancer treatment, wherein the compound selected from any one of items 1 to 199 or a pharmaceutically acceptable salt thereof.

1559. The compound for use for cancer treatment, wherein the compound selected from any one of items 1 to 199 or a pharmaceutically acceptable salt thereof, wherein compound is administered in combination with a treatment modality inducing DNA damage in cancer cells of said mammal.

1560. The compound for use of item 1559, wherein the treatment modality inducing DNA damage to cancer cells in said mammal comprises a radiotherapeutic treatment.

1561. The compound for use of item 1559, wherein the treatment modality inducing DNA damage to cancer cells in said mammal comprises a chemotherapeutic treatment.

1562. The compound for use of item 1559, wherein the treatment modality inducing DNA damage to cancer cells in said mammal comprises a radiotherapeutic and a chemotherapeutic treatment.

1563. The compound for use of item 1559, wherein the cancer is selected from cancer of the brain, lung, liver, spleen, kidney, lymph node, small intestine, pancreas, blood cells, colon, stomach, breast, endometrium, prostate, testicle, ovary, skin, head and neck, esophagus, bone marrow, and blood.

1564. The compound for use of item 1559, wherein the cancer is selected from breast cancer, lung cancer, prostate cancer, colorectal cancer, pancreatic cancer, haematological cancer and melanoma.

1565. The compound for use of item 1559, wherein the cancer is selected from pancreas cancer, prostate cancer and breast cancer.

1566. The compound for use of claim 1559, wherein the cancer is selected from cancer of the brain, lung, liver, spleen, kidney, lymph node, small intestine, pancreas, blood cells, colon, stomach, breast, endometrium, prostate, testicle, ovary, skin, head and neck, esophagus, bone marrow, and blood.

To avoid any doubts the wording “PFKFB3 inhibitor of any one of preceding items” and alike relates to compounds described in items 1 to 199, items 338 to 1545 and items A to H, meaning that the compound can be selected from any one of such items.

1567. A method of neuroprotection comprising deleting, reducing, binding, inhibiting or degrading PFKFB3 in cell of subject.

1568. A method of neuroprotection, comprising the administration of inhibitor of PFKFB3 kinase activity.

1569. A method of neuroprotection, comprising the administration of small molecule PFKFB3 inhibitor.

1570. A method of neuroprotection, comprising the administration of small molecule inhibitor of PFKFB3 kinase activity

1571. A method of neuroprotection comprising inhibiting PFKFB3 in cell of subject.

1572. A method of treatment or prophylaxis of neurodegenerative disease or neurodegenerative condition, comprising administering to a subject in need thereof an effective amount of PFKFB3 inhibitor.

1573. A method of treatment or prophylaxis of neurodegenerative disease or condition for which glycolysis inhibition has beneficial effect, comprising administering to a subject in need thereof an effective amount of PFKFB3 inhibitor or a pharmaceutical composition, comprising such PFKFB3 inhibitor.

1574. A method of increasing of cell antioxidant capacity, the method comprising contacting the cell with an effective amount of PFKFB3 inhibitor.

1575. A method for neuroprotection comprising administering to a subject in need thereof an effective amount of PFKFB3 inhibitor or a pharmaceutical composition, comprising such PFKFB3 inhibitor.

1576. A method of treatment of a neurodegenerative disease selected from Alzheimer's disease, amyotrophic lateral sclerosis, Huntington's disease, and Parkinson's disease, Late-onset Alzheimer disease, stroke, ataxia telangiectasia (Louis-Bar syndrome), argyrophilic grain disease, autosomal dominant cerebellar ataxia, Batten disease (Spielmeyer-Vogt-Sjögren-Batten disease), corticobasal degeneration, corticobasal ganglionic degeneration, progressive supranuclear palsy (Steele-Richardson-Olszewski syndrome), Creutzfeldt-Jakob disease, fatal familial insomnia, frontotemporal dementia and parkinsonism linked to chromosome 17, neuronal intermediate filament inclusion disease, basophilic inclusion body disease, Pick disease, dementia with Lewy bodies, multiple-system atrophy, hereditary motor and sensory neuropathy with proximal dominance, infantile refsum disease, Machado-Joseph disease, mental retardation and microcephaly with pontine and cerebellar hypoplasia (mental retardation, X-linked, syndromic, Najm type), neuroacanthocytosis, pontocerebellar hypoplasia, pyruvate dehydrogenase deficiency (pyruvate dehydrogenase complex deficiency), refsum disease (heredopathia atactica polyneuritiformis), abetalipoproteinemia (Bassen-Kornzweig syndrome), frontotemporal lobar degeneration, spinal muscular atrophy, Friedreich's ataxia, spinocerebellar ataxia, dentatorubral-pallidoluysian atrophy, Gerstmann-Sträussler-Scheinker syndrome, motor neurone disease, Charcot disease or Lou Gehrig's disease, sclerosis, spinal muscular atrophy, depression, bipolar disorder comprising administering to a subject in need thereof an effective amount of PFKFB3 inhibitor or a pharmaceutical composition, comprising such PFKFB3 inhibitor.

1577. A method of treatment of a neurodegenerative disease selected from Amyotrophic lateral sclerosis, Friedreich's ataxia, Huntington's disease, Lewy body disease, Parkinson's disease, Spinal muscular atrophy, Motor Neuron Diseases, Alpers' Disease, Cerebro-Oculo-Facio-Skeletal Syndrome (COFS) Corticobasal Degeneration, Gerstmann-Straussler-Scheinker Disease, Kuru, Leigh's Disease Monomelic Amyotrophy, Multiple System Atrophy, Multiple System Atrophy with Orthostatic Hypotension (Shy-Drager Syndrome), Neurodegeneration with Brain Iron Accumulation, Opsoclonus Myoclonus, Prion Diseases, Progressive Multifocal Leukoencephalopathy, Striatonigral Degeneration, Transmissible Spongiform Encephalopathies (Prion Diseases), Batten Disease, Alexander disease, Alpers-Huttenlocher syndrome, alpha-methylacyl-CoA racemase deficiency, Andermann syndrome, Arts syndrome, ataxia neuropathy spectrum, ataxia with oculomotor apraxia, autosomal dominant cerebellar ataxia, deafness, and narcolepsy, autosomal recessive spastic ataxia of Charlevoix-Saguenay, beta-propeller protein-associated neurodegeneration, CLN1 disease, CLN10 disease, CLN2 disease, CLN3 disease, CLN4 disease, CLN6 disease, CLN7 disease, CLN8 disease, congenital insensitivity to pain with anhidrosis, familial encephalopathy with neuroserpin inclusion bodies, fatty acid hydroxylase-associated neurodegeneration, GM2-gangliosidosis, AB variant, hereditary sensory and autonomic neuropathy type IE, hereditary sensory and autonomic neuropathy type II, hereditary sensory and autonomic neuropathy type V, infantile neuroaxonal dystrophy, infantile-onset ascending hereditary spastic paralysis, infantile-onset spinocerebellar ataxia, juvenile primary lateral sclerosis, Marinesco-Sjögren syndrome, mitochondrial membrane protein-associated neurodegeneration, multiple system atrophy, neuromyelitis optica, pantothenate kinase-associated neurodegeneration, polycystic lipomembranous osteodysplasia with sclerosing leukoencephalopathy, prion disease, progressive external ophthalmoplegia, riboflavin transporter deficiency neuronopathy, Sandhoff disease, spastic paraplegia type 49, comprising administering to a subject in need thereof an effective amount of PFKFB3 inhibitor or a pharmaceutical composition, comprising such PFKFB3 inhibitor.

1578. A method of treatment of a traumatic brain injury, comprising administering to a subject in need thereof an effective amount of PFKFB3 inhibitor or a pharmaceutical composition, comprising such PFKFB3 inhibitor.

1579. A method of decreasing a glycolytic uptake in neuron, comprising contacting the neuron with an effective amount of PFKFB3 inhibitor.

1580. A method of prevention of apoptotic death of neuron, comprising contacting the neuron with an effective amount of PFKFB3 inhibitor.

1581. A method of prevention of apoptotic death of neuron triggered by glutamate receptor over-activation, comprising administering to a subject in need thereof an effective amount of PFKFB3 inhibitor or a pharmaceutical composition comprising such PFKFB3 inhibitor.

1582. A method of prevention of apoptotic death of neuron triggered by glutamate receptor over-activation, comprising contacting the neuron with an effective amount of PFKFB3 inhibitor.

1583. A method of decreasing a glycolytic uptake in astrocyte, comprising contacting the astrocyte with an effective amount of PFKFB3 inhibitor.

1584. A method of inhibition reactive astrocyte proliferation comprising administering to a subject in need thereof an effective amount of PFKFB3 inhibitor or a pharmaceutical composition comprising such PFKFB3 inhibitor.

1585. A method of protection of neuron against excitotoxicity comprising administering to a subject in need thereof an effective amount of PFKFB3 inhibitor or a pharmaceutical composition comprising such PFKFB3 inhibitor.

1586. A method of protection of enteric neuron against excitotoxicity comprising administering to a subject in need thereof an effective amount of PFKFB3 inhibitor or a pharmaceutical composition comprising such PFKFB3 inhibitor.

1587. A method of protection of neuron against excitotoxicity comprising, comprising contacting the neuron with an effective amount of PFKFB3 inhibitor.

1588. A method of prophylaxis of a neurodegenerative disease comprising administering to a subject in need thereof an effective amount of PFKFB3 inhibitor or a pharmaceutical composition comprising such PFKFB3 inhibitor.

1589. A method of prophylaxis of a neurodegenerative disease selected from Alzheimer's disease, amyotrophic lateral sclerosis, stroke, Huntington's disease, and Parkinson's disease, Late-onset Alzheimer disease, ataxia telangiectasia (Louis-Bar syndrome), argyrophilic grain disease, autosomal dominant cerebellar ataxia, Batten disease (Spielmeyer-Vogt-Sjögren-Batten disease), corticobasal degeneration, corticobasal ganglionic degeneration, progressive supranuclear palsy (Steele-Richardson-Olszewski syndrome), Creutzfeldt-Jakob disease, fatal familial insomnia, frontotemporal dementia and parkinsonism linked to chromosome 17, neuronal intermediate filament inclusion disease, basophilic inclusion body disease, Pick disease, dementia with Lewy bodies, multiple-system atrophy, hereditary motor and sensory neuropathy with proximal dominance, infantile refsum disease, Machado-Joseph disease, mental retardation and microcephaly with pontine and cerebellar hypoplasia (mental retardation, X-linked, syndromic, Najm type), neuroacanthocytosis, pontocerebellar hypoplasia, pyruvate dehydrogenase deficiency (pyruvate dehydrogenase complex deficiency), refsum disease (heredopathia atactica polyneuritiformis), abetalipoproteinemia (Bassen-Kornzweig syndrome), frontotemporal lobar degeneration, spinal muscular atrophy, Friedreich's ataxia, spinocerebellar ataxia, dentatorubral-pallidoluysian atrophy, Gerstmann-Sträussler-Scheinker syndrome, motor neurone disease, Charcot disease or Lou Gehrig's disease, sclerosis, spinal muscular atrophy, depression, and bipolar disorder comprising administering to a subject in need thereof an effective amount of PFKFB3 inhibitor or a pharmaceutical composition comprising such PFKFB3 inhibitor.

1590. A method for conferring neuroprotection on a population of cells in a subject, the method comprising: administering the PFKFB3 inhibitor in an effective dose to the population of cells so as to confer neuroprotection.

1591. A method of treating a human subject following acute central nervous system injury, the method comprising administering a pharmaceutical composition comprising at least one PFKFB3 inhibitor to said human within a predetermined time period following said acute central nervous system injury.

1592. The method of preceding item, wherein said chronic central nervous system injury is caused by a neurodegenerative disease.

1593. The method of preceding item, wherein a neurodegenerative disease is selected from any one of preceding items.

1594. A method of attenuating or preventing neuronal damage in a human subject at risk of chronic central nervous system injury, the method comprising administering a pharmaceutical composition comprising at least PFKFB3 inhibitor to said human subject prior to said chronic central nervous system injury.

1595. The method of preceding item, wherein said chronic central nervous system injury is caused by a neurodegenerative disease.

1596. The method of preceding item, wherein a neurodegenerative disease is selected from any one of preceding items.

1597. A method of any one of preceding items, wherein said method further comprises attenuating neuronal damage in the human subject.

1598. A method of manufacturing a neuroprotection medication, comprising the use of PFKFB3 inhibitor as an active ingredient.

1599. The compound of any one of items 1-199 or a pharmaceutical composition of any one of items 200 to 203 for use in treatment of cerebral ischemia.

1600. The compound of any one of items 1-199 or a pharmaceutical composition of any one of items 200 to 203 for use in treatment of neurological insult.

1601. The compound of any one of items 1-199 or a pharmaceutical composition of any one of items 200 to 203 for use in treatment of ischemic stroke.

1602. The compound of any one of items 1-199 or a pharmaceutical composition of any one of items 200 to 203 for use in treatment of neonatal ischemic stroke.

1603. The compound of any one of items 1-199 or a pharmaceutical composition of any one of items 200 to 203 for use in treatment of transient ischemic attack

1604. A method of treatment of cerebral ischemia, comprising administering of PFKFB3 inhibitor, wherein PFKFB3 inhibitor is selected from any one of the items 1 to 199 or a pharmaceutical composition of any one of items 200 to 203.

1605. A method of treatment of neurological insult, comprising administering of PFKFB3 inhibitor, wherein PFKFB3 inhibitor is selected from any one of the items 1 to 199 or a pharmaceutical composition of any one of items 200 to 203.

1606. A method of treatment of ischemic stroke, comprising administering of PFKFB3 inhibitor, wherein PFKFB3 inhibitor is selected from any one of the items 1 to 199 or a pharmaceutical composition of any one of items 200 to 203.

1607. A method of treatment of neonatal ischemic stroke, comprising administering of PFKFB3 inhibitor, wherein PFKFB3 inhibitor is selected from any one of the items 1 to 199 or a pharmaceutical composition of any one of items 200 to 203.

1608. The compound selected from any one of items any one of the items 338 to 1545 or any one of the items A,B,C,D,E, F, G or H for use in treatment of cerebral ischemia.

1609. The compound selected from any one of items any one of the items 338 to 1545 or any one of the items A,B,C,D,E, F, G or H for use in treatment of neurological insult.

1610. The compound selected from any one of items any one of the items 338 to 1545 or any one of the items A,B,C,D,E, F, G or H for use in treatment of ischemic stroke.

1611. The compound selected from any one of items any one of the items 338 to 1545 or any one of the items A,B,C,D,E, F, G or H for use in treatment of neonatal ischemic stroke.

1612. The compound selected from any one of items any one of the items 338 to 1545 or any one of the items A,B,C,D,E, F, G or H for use in treatment of transient ischemic attack.

1613. A method of treatment of cerebral ischemia, comprising administering of PFKFB3 inhibitor, wherein PFKFB3 inhibitor is selected from any one of items any one of the items 338 to 1545 or any one of the items A,B,C,D,E, F, G or H.

1614. A method of treatment of neurological insult, comprising administering of PFKFB3 inhibitor, wherein PFKFB3 inhibitor is selected from any one of items any one of the items 338 to 1545 or any one of the items A,B,C,D,E, F, G or H.

1615. A method of treatment of ischemic stroke, comprising administering of PFKFB3 inhibitor, wherein PFKFB3 inhibitor is selected from any one of items any one of the items 338 to 1545 or any one of the items A,B,C,D,E, F, G or H.

1616. A method of treatment of neonatal ischemic stroke, comprising administering of PFKFB3 inhibitor, wherein PFKFB3 inhibitor is selected from any one of items any one of the items 338 to 1545 or any one of the items A,B,C,D,E, F, G or H.

Anti-Aging

1617. A method of treatment or preventing an age-related disease or disorder or other anti-aging treatment comprising deleting, reducing, binding, inhibiting or degrading of PFKFB3 in cell of subject.

1618. A method of preceding item, comprising administering by subject a medication and wherein deleting, reducing, binding, inhibiting or degrading of PFKFB3 is achieved by such medication.

1619. A method of treatment or preventing an age-related disease or disorder or other anti-aging treatment comprising administering to a subject in need thereof a PFKFB3 inhibitor or a pharmaceutical composition, comprising PFKFB3 inhibitor.

1620. A method of treating or preventing an age-related disease or disorder or other anti-aging treatment comprising administering to a subject in need thereof a PFKFB3 inhibitor, a modulator, an inhibitor, a degradation agent of Indirect Target.

1621. A method of treatment or preventing an age-related disease or disorder or other anti-aging treatment comprising administering to a subject in need thereof a pharmaceutical composition of any of preceding items.

1622. A method of maintaining or improving health of a subject comprising a step of administering in a subject a PFKFB3 inhibitor or of a composition comprising a PFKFB3 inhibitor as an active agent.

1623. A method of maintaining or improving fitness of a subject comprising a step of administering in a subject a PFKFB3 inhibitor or of a composition comprising a PFKFB3 inhibitor as an active agent.

1624. Method of improving/increasing activity in a subject comprising a step of administering in a subject a PFKFB3 inhibitor or of a composition comprising a PFKFB3 inhibitor as an active agent.

1625. Method of improving/increasing functional activity in a subject comprising a step of administering in a subject a PFKFB3 inhibitor or of a composition comprising a PFKFB3 inhibitor as an active agent.

1626. Method of improving a parameter selected from: muscle strength, bone density, hair growth, cognitive performance, stress resistance or resilience, blood parameters, heart rate, cognitive functions, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), time for recovery after any stress (wound, operation, chemotherapy, disease, change in lifestyle etc.), Standing height, Forced expiratory volume in 1-second (FEV1), Leg fat-free mass (right), Leg predicted mass (right), Basal metabolic rate, Forced vital capacity (FVC), Leg fat-free mass (left), Leg predicted mass (left), Systolic blood pressure, automated reading, Heel bone mineral density (BMD) (left), Heel quantitative ultrasound index (QUI), direct entry (left), Whole body fat-free mass, Whole body water mass, Heel bone mineral density (BMD) T-score, automated (left), Speed of sound through heel (left), Sitting height, Heel bone mineral density (BMD) (right), Heel quantitative ultrasound index (QUI), direct entry (right), Speed of sound through heel (right), Heel bone mineral density (BMD) T-score, automated (right), Peak expiratory flow (PEF), Leg fat percentage (left), Trunk fat-free mass, Leg fat percentage (right), Trunk predicted mass, Hand grip strength (left), Heel broadband ultrasound attenuation (left), Heel broadband ultrasound attenuation (right), Hand grip strength (right), Duration to first press of snap-button in each round, Mean time to correctly identify matches, Body fat percentage Trunk fat percentage, Body mass index (BMI), Leg fat mass (left), Arm fat-free mass (left), Arm predicted mass (left), Arm fat-free mass (right), Haematocrit percentage, Arm predicted mass (right), Waist circumference, Leg fat mass (right), Haemoglobin concentration, Arm fat percentage (left), Ankle spacing width (left), Whole body fat mass, Body mass index (BMI), Pulse wave peak to peak time, Arm fat percentage (right), Weight, Mean corpuscular volume, Trunk fat mass, Pulse wave Arterial Stiffness index Ankle spacing width (right), Platelet crit, Red blood cell (erythrocyte) count, Mean sphered cell volume, Mean platelet (thrombocyte) volume, Weight, Arm fat mass (left), Lymphocyte percentage, Neutrophill percentage, Arm fat mass (right), Impedance of leg (left), Mean reticulocyte volume, Platelet count, Mean corpuscular haemoglobin, Impedance of leg (right), Red blood cell (erythrocyte) distribution width, Pulse rate, automated reading, Impedance of whole body, Diastolic blood pressure, automated reading, Lymphocyte count, Number of measurements made, Neutrophill count, Monocyte percentage, Hip circumference, Monocyte count, Platelet distribution width, Mean corpuscular haemoglobin concentration, Immature reticulocyte fraction, Impedance of arm (right), Reticulocyte percentage, Number of times snap-button pressed, White blood cell (leukocyte) count, Pulse rate, High light scatter reticulocyte count, Basophill percentage, Impedance of arm (left), Pulse wave reflection index, Eosinophill count Nucleated red blood cell count, Eosinophill percentage, Basophill count, Reticulocyte count, High light scatter reticulocyte percentage, Nucleated red blood cell percentage, or any one other parameter worsening with the age comprising a step of administering in a subject a PFKFB3 inhibitor or of a composition comprising a PFKFB3 inhibitor as an active agent.

1627. Method of improving at least two of parameters described in preceding item comprising a step of administering in a subject a PFKFB3 inhibitor or of a composition comprising a PFKFB3 inhibitor as an active agent.

1628. Method of improving at least two of health parameters worsening with the age comprising a step of administering in a subject a PFKFB3 inhibitor or of a composition comprising a PFKFB3 inhibitor as an active agent.

1629. Method of anti-aging treatment comprising a step of administering in a subject a PFKFB3 inhibitor or of a composition comprising a PFKFB3 inhibitor as an active agent.

1630. Method of prevention, amelioration or lessening the effects of aging comprising a step of administering in a subject a PFKFB3 inhibitor or of a composition comprising a PFKFB3 inhibitor as an active agent.

1631. Method of decreasing or delaying an increase in the biological age or slowing rate of aging comprising a step of administering in a subject a PFKFB3 inhibitor or of a composition comprising a PFKFB3 inhibitor as an active agent.

1632. Method of the changing biomarker or biomarkers of morbidity into the state corresponding to less chances of morbidity comprising a step of administering in a subject a PFKFB3 inhibitor.

1633. Method of treatment, prevention, amelioration and lessening the effects of frailty comprising a step of administering in a subject a PFKFB3 inhibitor or of a composition comprising a PFKFB3 inhibitor as an active agent.

1634. Method of the treatment of aging related disease comprising a step of administering in a subject a PFKFB3 inhibitor or of a composition comprising a PFKFB3 inhibitor as an active agent.

1635. Method of the increasing health span or lifespan, comprising a step of administering in a subject a PFKFB3 inhibitor or of a composition comprising a PFKFB3 inhibitor as an active agent.

1636. Method of the rejuvenation, comprising a step of administering in a subject a PFKFB3 inhibitor or of a composition comprising a PFKFB3 inhibitor as an active agent.

1637. Method of the at least one of the following: increasing stress resistance or resilience, increasing rate or other enhancement of recovery after surgery, radiotherapy, disease and/or any other stress comprising a step of administering in a subject a PFKFB3 inhibitor or of a composition comprising a PFKFB3 inhibitor as an active agent.

1638. Method of the prevention and/or the treatment of menopausal syndrome or restoring reproductive function, comprising a step of administering in a subject a PFKFB3 inhibitor or of a composition comprising a PFKFB3 inhibitor as an active agent.

1639. Method of the eliminating or decrease in spreading of senescent cells, comprising a step of administering in a subject a PFKFB3 inhibitor or of a composition comprising a PFKFB3 inhibitor as an active agent.

1640. Method of the decreasing all-causes or multiple causes of mortality risks or mortality risks related to at least one or at least two of age related diseases or conditions or delaying in increase of such risks, decreasing morbidity risks comprising a step of administering in a subject a PFKFB3 inhibitor or of a composition comprising a PFKFB3 inhibitor as an active agent.

1641. Method of the modulating at least one of biomarkers of aging into more youthful state or slowing down its change into “elder” state comprising a step of administering in a subject a PFKFB3 inhibitor or of a composition comprising a PFKFB3 inhibitor as an active agent.

1642. Method of the prevention or treatment of aging related disease, comprising a step of administering in a subject a PFKFB3 inhibitor or of a composition comprising a PFKFB3 inhibitor as an active agent.

1643. Method of the prevention or treatment of aging related disease, selected from: atherosclerosis, cardiovascular disease, cachexia, arthritis, cataracts, osteoporosis, type 2 diabetes, hypertension, neurodegeneration (including but not limited to Alzheimer's disease, dementia, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence (including but not limited to age related decline in immune response to vaccines, age related decline in response to immunotherapy etc.), myocardial infarction, acute coronary syndrome, sarcopenia, sarcopenic obesity, senile osteoporosis, urinary incontinence or any one of others aging related diseases comprising a step of administering in a subject a PFKFB3 inhibitor or of a composition comprising a PFKFB3 inhibitor as an active agent.

1644. Method of treatment of accelerated aging comprising a step of administering in a subject a PFKFB3 inhibitor or of a composition comprising a PFKFB3 inhibitor as an active agent.

1645. Method of treatment of accelerated aging of cancer survivor comprising a step of administering in a subject a PFKFB3 inhibitor or of a composition comprising a PFKFB3 inhibitor as an active agent.

1646. Method of treatment of accelerated aging of subject suffering from HIV comprising a step of administering in a subject a PFKFB3 inhibitor or of a composition comprising a PFKFB3 inhibitor as an active agent.

1647. Method of the prevention or treatment of consequences of chemotherapy comprising a step of administering in a subject a PFKFB3 inhibitor or of a composition comprising a PFKFB3 inhibitor as an active agent.

1648. Method of the prevention or treatment of consequences of radiotherapy comprising a step of administering in a subject a PFKFB3 inhibitor or of a composition comprising a PFKFB3 inhibitor as an active agent.

1649. Method of the radioprotection comprising a step of administering in a subject a PFKFB3 inhibitor or of a composition comprising a PFKFB3 inhibitor as an active agent.

1650. Method of the changing biomarker or biomarkers of all-cause mortality into the state corresponding to less chances of mortality comprising a step of administering in a subject a PFKFB3 inhibitor or of a composition comprising a PFKFB3 inhibitor as an active agent.

1651. Method of the changing biomarker or biomarkers of mortality into the state corresponding to less chances of mortality comprising a step of administering in a subject a PFKFB3 inhibitor or of a composition comprising a PFKFB3 inhibitor as an active agent.

1652. Method of the changing biomarker or biomarkers of health span or marker of life expectancy into the state corresponding to longer health span or life expectancy comprising a step of administering in a subject a PFKFB3 inhibitor or of a composition comprising a PFKFB3 inhibitor as an active agent.

1653. A method of manufacturing of anti-aging therapy comprising a step of using a PFKFB3 inhibitor as an active agent.

1654. A method of manufacturing of therapy for a use of any one of the items of this application comprising a step of using a PFKFB3 inhibitor as an active agent.

1655. A method of any one of preceding items, wherein such method is applied to a healthy subject.

1656. A method of any one of preceding items, wherein such method is applied to an elderly subject

1657. A method of any one of preceding items, wherein such method is applied to a subject of an age of >40 years.

1658. A method of any one of preceding items, wherein such method is applied to a subject of an age of >50 years.

1659. A method of any one of preceding items, wherein such method is applied to a subject of an age of >60 years.

1660. A method of any one of preceding items, wherein such method is applied to subject who shows symptoms of ageing.

1661. A method of any one of preceding items, wherein such method is applied to a subject who does not suffer from an age-related disease or disorder.

1662. A method of any one of preceding items, wherein said method is a non-therapeutic.

1663. A method of any one of the preceding items wherein PFKFB3 inhibitor, modulator or degradation agent is selected from peptide, small molecule, antibody, aptamer, protein, virus, polymer, gene therapy, nanoparticle or particle.

1664. A method of any of the preceding items wherein instead of PFKFB3 inhibitor a modulator of Indirect Target is used.

1665. A method of preceding item, wherein modulation of Indirect Target mimics or effects the reduction or inhibition of PFKFB3.

1666. A method of any one of preceding items, wherein instead of PFKFB3 inhibitor a composition, comprising PFKFB3 inhibitor is used.

1667. A method of preceding item wherein composition further comprises at least one pharmaceutically acceptable excipient.

1668. A method of any one of preceding items, wherein PFKFB3 inhibitor is a small molecule PFKFB3 inhibitor.

1669. A method of any one of preceding items, wherein PFKFB3 inhibitor is a small molecule inhibitor of PFKFB3 kinase activity.

1670. A method of any one of preceding items, wherein PFKFB3 inhibitor is in therapeutically effective amount.

1671. A method of any one of preceding items, wherein PFKFB3 inhibitor is administered in pharmaceutical composition, further comprising at least one pharmaceutically acceptable excipient.

1672. A method of any one of preceding items, wherein PFKFB3 age related disease or disorder excludes cancer

1673. A method of any one of preceding items, wherein PFKFB3 inhibitor is selected from any one of the items below.

1674. An agent deleting, reducing, binding, inhibiting or degrading PFKFB3 in cell of subject PFKFB3 inhibitor for use in neuroprotection.

1675. A PFKFB3 inhibitor for use as neuroprotector.

1676. A small molecule PFKFB3 kinase activity inhibitor for use as neuroprotector

1677. A PFKFB3 kinase activity inhibitor for use in neuroprotection.

1678. A PFKFB3 small molecule inhibitor for use in neuroprotection

1679. A small molecule inhibitor of PFKFB3 kinase activity for use in neuroprotection.

1680. A PFKFB3 inhibitor for use in treatment or prophylaxis of neurodegenerative disease or neurodegenerative condition.

1681. A PFKFB3 inhibitor for use in treatment of a neurodegenerative disease selected from Alzheimer's disease, amyotrophic lateral sclerosis, Huntington's disease, and Parkinson's disease, Late-onset Alzheimer disease, stroke, ataxia telangiectasia (Louis-Bar syndrome), argyrophilic grain disease, autosomal dominant cerebellar ataxia, Batten disease (Spielmeyer-Vogt-Sjögren-Batten disease), corticobasal degeneration, corticobasal ganglionic degeneration, progressive supranuclear palsy (Steele-Richardson-Olszewski syndrome), Creutzfeldt-Jakob disease, fatal familial insomnia, frontotemporal dementia and parkinsonism linked to chromosome 17, neuronal intermediate filament inclusion disease, basophilic inclusion body disease, Pick disease, dementia with Lewy bodies, multiple-system atrophy, hereditary motor and sensory neuropathy with proximal dominance, infantile refsum disease, Machado-Joseph disease, mental retardation and microcephaly with pontine and cerebellar hypoplasia (mental retardation, X-linked, syndromic, Najm type), neuroacanthocytosis, pontocerebellar hypoplasia, pyruvate dehydrogenase deficiency (pyruvate dehydrogenase complex deficiency), refsum disease (heredopathia atactica polyneuritiformis), abetalipoproteinemia (Bassen-Kornzweig syndrome), frontotemporal lobar degeneration, spinal muscular atrophy, Friedreich's ataxia, spinocerebellar ataxia, dentatorubral-pallidoluysian atrophy, Gerstmann-Sträussler-Scheinker syndrome, motor neurone disease, Charcot disease or Lou Gehrig's disease, sclerosis, spinal muscular atrophy, depression, bipolar disorder, Amyotrophic lateral sclerosis, Friedreich's ataxia, Huntington's disease, Lewy body disease, Parkinson's disease, Spinal muscular atrophy, Motor Neuron Diseases, Alpers' Disease, Cerebro-Oculo-Facio-Skeletal Syndrome (COFS), Corticobasal Degeneration Gerstmann-Straussler-Scheinker Disease, Kuru, Leigh's Disease, Monomelic Amyotrophy, Multiple System Atrophy, Multiple System Atrophy with Orthostatic Hypotension (Shy-Drager Syndrome) Neurodegeneration with Brain Iron Accumulation, Opsoclonus Myoclonus, Prion Diseases, Progressive Multifocal Leukoencephalopathy, Striatonigral Degeneration, Transmissible Spongiform Encephalopathies (Prion Diseases), Batten Disease, Alexander disease, Alpers-Huttenlocher syndrome, alpha-methylacyl-CoA racemase deficiency, Andermann syndrome, Arts syndrome, ataxia neuropathy spectrum, ataxia with oculomotor apraxia, autosomal dominant cerebellar ataxia, deafness, and narcolepsy, autosomal recessive spastic ataxia of Charlevoix-Saguenay, beta-propeller protein-associated neurodegeneration, CLN1 disease, CLN10 disease, CLN2 disease, CLN3 disease, CLN4 disease, CLN6 disease, CLN7 disease, CLN8 disease, congenital insensitivity to pain with anhidrosis, familial encephalopathy with neuroserpin inclusion bodies, fatty acid hydroxylase-associated neurodegeneration, GM2-gangliosidosis, AB variant, hereditary sensory and autonomic neuropathy type IE, hereditary sensory and autonomic neuropathy type II, hereditary sensory and autonomic neuropathy type V, infantile neuroaxonal dystrophy, infantile-onset ascending hereditary spastic paralysis, infantile-onset spinocerebellar ataxia, juvenile primary lateral sclerosis, Marinesco-Sjögren syndrome, mitochondrial membrane protein-associated neurodegeneration, multiple system atrophy, neuromyelitis optica, pantothenate kinase-associated neurodegeneration, polycystic lipomembranous osteodysplasia with sclerosing leukoencephalopathy, prion disease, progressive external ophthalmoplegia, riboflavin transporter deficiency neuronopathy, Sandhoff disease, spastic paraplegia type 49.

1682. A PFKFB3 inhibitor for use in treatment of Alzheimer's disease.

1683. A PFKFB3 inhibitor for use in treatment of amyotrophic lateral sclerosis.

1684. A PFKFB3 inhibitor for use in treatment of Huntington's disease.

1685. A PFKFB3 inhibitor for use in treatment of Parkinson's disease.

1686. A PFKFB3 inhibitor for use in treatment of neuropathy.

1687. A PFKFB3 inhibitor for use in treatment of multiple sclerosis.

1688. A PFKFB3 inhibitor for use in treatment of a traumatic brain injury.

1689. A PFKFB3 inhibitor for use in prophylaxis of a neurodegeneration.

1690. A PFKFB3 inhibitor for use in prophylaxis of a neurodegenerative disease selected from Alzheimer's disease, amyotrophic lateral sclerosis, stroke, Huntington's disease, and Parkinson's disease, Late-onset Alzheimer disease, ataxia telangiectasia (Louis-Bar syndrome), argyrophilic grain disease, autosomal dominant cerebellar ataxia, Batten disease (Spielmeyer-Vogt-Sjögren-Batten disease), corticobasal degeneration, corticobasal ganglionic degeneration, progressive supranuclear palsy (Steele-Richardson-Olszewski syndrome), Creutzfeldt-Jakob disease, fatal familial insomnia, frontotemporal dementia and parkinsonism linked to chromosome 17, neuronal intermediate filament inclusion disease, basophilic inclusion body disease, Pick disease, dementia with Lewy bodies, multiple-system atrophy, hereditary motor and sensory neuropathy with proximal dominance, infantile refsum disease, Machado-Joseph disease, mental retardation and microcephaly with pontine and cerebellar hypoplasia (mental retardation, X-linked, syndromic, Najm type), neuroacanthocytosis, pontocerebellar hypoplasia, pyruvate dehydrogenase deficiency (pyruvate dehydrogenase complex deficiency), refsum disease (heredopathia atactica polyneuritiformis), abetalipoproteinemia (Bassen-Kornzweig syndrome), frontotemporal lobar degeneration, spinal muscular atrophy, Friedreich's ataxia, spinocerebellar ataxia, dentatorubral-pallidoluysian atrophy, Gerstmann-Sträussler-Scheinker syndrome, motor neurone disease, Charcot disease or Lou Gehrig's disease, sclerosis, spinal muscular atrophy, depression, and bipolar disorder.

1691. A PFKFB3 inhibitor for use in conferring neuroprotection on a population of cells in a subject.

1692. A PFKFB3 inhibitor for use in manufacturing a neuroprotection medication, comprising the use of PFKFB3 inhibitor as an active ingredient.

1693. An agent deleting, reducing, binding, inhibiting or degrading PFKFB3 in cell of subject PFKFB3 inhibitor for use in use in treatment or preventing an age-related disease or disorder or other anti-aging treatment.

1694. A PFKFB3 inhibitor for use in treatment or preventing an age-related disease or disorder or other anti-aging treatment.

1695. A PFKFB3 inhibitor for use in treating or preventing an age-related disease or disorder or other anti-aging treatment.

1696. A PFKFB3 inhibitor for use in maintaining or improving health of a subject.

1697. A PFKFB3 inhibitor for use in maintaining or improving fitness of a subject.

1698. A PFKFB3 inhibitor for use in improving/increasing activity in a subject.

1699. A PFKFB3 inhibitor for use in improving/increasing functional activity in a subject.

1700. A PFKFB3 inhibitor for use in improving a parameter selected from: muscle strength, bone density, hair growth, cognitive performance, stress resistance or resilience, blood parameters, heart rate, cognitive functions, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), time for recovery after any stress (wound, operation, chemotherapy, disease, change in lifestyle etc.), Standing height, Forced expiratory volume in 1-second (FEV1), Leg fat-free mass (right), Leg predicted mass (right), Basal metabolic rate, Forced vital capacity (FVC), Leg fat-free mass (left), Leg predicted mass (left), Systolic blood pressure, automated reading, Heel bone mineral density (BMD) (left), Heel quantitative ultrasound index (QUI), direct entry (left), Whole body fat-free mass, Whole body water mass, Heel bone mineral density (BMD) T-score, automated (left), Speed of sound through heel (left), Sitting height, Heel bone mineral density (BMD) (right), Heel quantitative ultrasound index (QUI), direct entry (right), Speed of sound through heel (right), Heel bone mineral density (BMD) T-score, automated (right), Peak expiratory flow (PEF), Leg fat percentage (left), Trunk fat-free mass, Leg fat percentage (right), Trunk predicted mass Hand grip strength (left), Heel broadband ultrasound attenuation (left), Heel broadband ultrasound attenuation (right), Hand grip strength (right), Duration to first press of snap-button in each round, Mean time to correctly identify matches, Body fat percentage Trunk fat percentage, Body mass index (BMI), Leg fat mass (left), Arm fat-free mass (left), Arm predicted mass (left), Arm fat-free mass (right), Haematocrit percentage, Arm predicted mass (right), Waist circumference, Leg fat mass (right), Haemoglobin concentration, Arm fat percentage (left), Ankle spacing width (left), Whole body fat mass, Body mass index (BMI), Pulse wave peak to peak time, Arm fat percentage (right), Weight, Mean corpuscular volume, Trunk fat mass, Pulse wave Arterial Stiffness index Ankle spacing width (right), Platelet crit, Red blood cell (erythrocyte) count, Mean sphered cell volume Mean platelet (thrombocyte) volume, Weight, Arm fat mass (left), Lymphocyte percentage, Neutrophill percentage, Arm fat mass (right), Impedance of leg (left), Mean reticulocyte volume, Platelet count, Mean corpuscular haemoglobin, Impedance of leg (right), Red blood cell (erythrocyte) distribution width, Pulse rate, automated reading, Impedance of whole body, Diastolic blood pressure, automated reading, Lymphocyte count, Number of measurements made, Neutrophill count, Monocyte percentage Hip circumference, Monocyte count, Platelet distribution width, Mean corpuscular haemoglobin concentration, Immature reticulocyte fraction, Impedance of arm (right), Reticulocyte percentage, Number of times snap-button pressed, White blood cell (leukocyte) count, Pulse rate, High light scatter reticulocyte count, Basophill percentage, Impedance of arm (left), Pulse wave reflection index, Eosinophill count Nucleated red blood cell count, Eosinophill percentage, Basophill count, Reticulocyte count, High light scatter reticulocyte percentage, Nucleated red blood cell percentage, or any one other parameter worsening with the age.

1701. A PFKFB3 inhibitor for use in improving at least two of parameters described in preceding item.

1702. A PFKFB3 inhibitor for use in improving at least two of health parameters worsening with the age.

1703. A PFKFB3 inhibitor for use in anti-aging treatment.

1704. A PFKFB3 inhibitor for use in prevention, amelioration or lessening the effects of aging.

1705. A PFKFB3 inhibitor for use in decreasing or delaying an increase in the biological age or slowing rate of aging.

1706. A PFKFB3 inhibitor for use in treatment, prevention, amelioration and lessening the effects of frailty.

1707. A PFKFB3 inhibitor for use in the treatment of aging related disease.

1708. A PFKFB3 inhibitor for use in the increasing health span or lifespan.

1709. A PFKFB3 inhibitor for use in the rejuvenation.

1710. A PFKFB3 inhibitor for use in the at least one of the following: increasing stress resistance or resilience, increasing rate or other enhancement of recovery after surgery, radiotherapy, disease and/or any other stress.

1711. A PFKFB3 inhibitor for use in the prevention and/or the treatment of menopausal syndrome or restoring reproductive function.

1712. A PFKFB3 inhibitor for use in the eliminating or decrease in spreading of senescent cells.

1713. A PFKFB3 inhibitor for use in the decreasing all-causes or multiple causes of mortality risks or mortality risks related to at least one or at least two of age related diseases or conditions or delaying in increase of such risks, decreasing morbidity risks.

1714. A PFKFB3 inhibitor for use in the changing biomarker or biomarkers of morbidity into the state corresponding to less chances of morbidity

1715. A PFKFB3 inhibitor for use in the modulating at least one of biomarkers of aging into more youthful state or slowing down its change into “elder” state.

1716. A PFKFB3 inhibitor for use in the prevention or treatment of aging related disease.

1717. A PFKFB3 inhibitor for use in the prevention or treatment of aging related disease, selected from: atherosclerosis, cardiovascular disease, cachexia, arthritis, cataracts, osteoporosis, type 2 diabetes, hypertension, neurodegeneration (including but not limited to Alzheimer's disease, dementia, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence (including but not limited to age related decline in immune response to vaccines, age related decline in response to immunotherapy etc.), myocardial infarction, acute coronary syndrome, sarcopenia, sarcopenic obesity, senile osteoporosis, urinary incontinence etc.

1718. A PFKFB3 inhibitor for use in treatment of accelerated aging.

1719. A PFKFB3 inhibitor for use in treatment of accelerated aging of cancer survivor.

1720. A PFKFB3 inhibitor for use in treatment of accelerated aging of subject suffering from HIV.

1721. A PFKFB3 inhibitor for use in the prevention or treatment of consequences of chemotherapy.

1722. A PFKFB3 inhibitor for use in the prevention or treatment of consequences of radiotherapy.

1723. A PFKFB3 inhibitor for use in the radioprotection.

1724. A PFKFB3 inhibitor for use in the changing biomarker or biomarkers of all-cause mortality into the state corresponding to less chances of mortality.

1725. A PFKFB3 inhibitor for use in the changing biomarker or biomarkers of mortality into the state corresponding to less chances of mortality.

1726. A PFKFB3 inhibitor for use in the changing biomarker or biomarkers of health span or marker of life expectancy into the state corresponding to longer health span or life expectancy.

1727. A PFKFB3 inhibitor for use in manufacturing of anti-aging therapy.

1728. A PFKFB3 inhibitor for use of any one of preceding items, wherein such use is in healthy subject.

1729. A PFKFB3 inhibitor for use of any one of preceding items, wherein use is in an elderly subject 1730. A PFKFB3 inhibitor for use of any one of preceding items, wherein use is in a subject of an age of >40 years.

1731. A PFKFB3 inhibitor for use of any one of preceding items, wherein such use is in a subject of an age of >50 years.

1732. A PFKFB3 inhibitor for use of any one of preceding items, wherein such use is in a subject of an age of >60 years.

1733. A PFKFB3 inhibitor for use of any one of preceding items, wherein such use is in subject who shows symptoms of ageing.

1734. A PFKFB3 inhibitor for use of any one of preceding items, wherein such use is in a subject who does not suffer from an age-related disease or disorder.

1735. A PFKFB3 inhibitor for use of any one of preceding items, wherein said use is in a non-therapeutic.

1736. A PFKFB3 inhibitor of any of preceding items, wherein instead of A PFKFB3 inhibitor a composition comprising a PFKFB3 inhibitor as an active agent is used.

1737. A PFKFB3 inhibitor for use in preceding item wherein composition further comprises at least one pharmaceutically acceptable excipient.

1738. Neuroprotective pharmaceutical composition, comprising a PFKFB3 inhibitor and at least one pharmaceutically acceptable excipient.

1739. Anti-aging pharmaceutical composition, comprising a PFKFB3 inhibitor and at least one pharmaceutically acceptable excipient.

1740. Pharmaceutical composition for the use of any one of the preceding items, comprising a PFKFB3 inhibitor, and at least one pharmaceutically acceptable excipient. For clarity, for example, a item “A PFKFB3 inhibitor for use in treatment of accelerated aging” for a purpose of this item will give a new item “Pharmaceutical composition for the use in treatment of accelerated aging, comprising a PFKFB3 inhibitor”.

1741. Anti-aging pharmaceutical composition, comprising PFKFB3 inhibitor and at least one pharmaceutically acceptable excipient.

1742. Anti-aging pharmaceutical composition, comprising modulator of Indirect Target and at least one pharmaceutically acceptable excipient.

1743. Pharmaceutical composition for the use of any one of the preceding items, comprising modulator of Indirect Target, and at least one pharmaceutically acceptable excipient.

1744. Composition of any one of preceding items, wherein modulator of Indirect Target mimics or effects the reduction or inhibition of PFKFB3.

1745. A method of testing or controlling of the efficacy of therapy selected from PFKFB3 silencing therapy, PFKFB3 deleting therapy, therapy reducing PFKFB3, therapy binding PFKFB3, therapy inhibiting PFKFB3 or therapy degrading PFKFB3, comprising a step of checking in the subject treated by such therapy a parameter selected from the following: biological age of the patient, at least one aging biomarker, at least one age related deficit or disease, at least one of rejuvenation marker, frailty, health span or lifespan, or any other marker or parameter reasonable for checking in testing of anti-aging therapy efficacy.

1746. A method of preceding item, wherein the therapy is not an anti-cancer therapy.

1747. A method of testing or controlling of the efficacy of PFKFB3 inhibitor or pharmaceutical, composition, comprising such inhibitor, wherein checking of efficacy of therapy/measurement of markers or symptoms of related diseases or conditions is conducted after 1 month after the administration of therapy in therapeutically effective amount, after 3 months, after 6 months, after 12 months, after 18 months, after 24 months or after 36 months after such administration, or in around such date, or in date reasonably defined by the doctor based on the parameter being measured and other factors known to the expert in the field.

1748. A method of testing or controlling of the efficacy of any of preceding items, wherein PFKFB3 inhibitor is a monoclonal or polyclonal antibody, optionally humanized, protein, aptamer, peptide, polymer, virus or small molecule, a compound with a specificity for a PFKFB3 polynucleotide selected from the list consisting of a small interfering RNA (siRNA), an artificial microRNA, an antisense polynucleotide or a ribozyme, silencing binding or inhibiting or degrading a PFKFB3 or any molecule or composition described in this disclosure or its analog.

1749. A method of testing or controlling of the efficacy of therapy of any of preceding items, wherein therapy is a modulator of at least one of Indirect Targets, wherein such modulation has an anti-aging effect.

1750. A method of testing or controlling of the efficacy of PFKFB3 small molecule inhibitor, comprising a step of checking neuroprotective effect of such inhibitor in cell.

1751. A method of testing or controlling of the efficacy of PFKFB3 small molecule inhibitor, comprising a step of checking neuroprotective effect of such inhibitor in subject treated by such inhibitor.

1752. A method of testing or controlling of the efficacy of any of preceding items, wherein therapy comprises at least one of the compounds described in any one of preceding items or in this application.

Kit

1753. A kit for neuroprotection, comprising PFKFB3 inhibitor and instruction for use.

1754. A kit for anti-aging treatment, comprising PFKFB3 inhibitor and instruction for use.

1755. A kit, comprising PFKFB3 inhibitor and instructions for use wherein use is selected from any one of preceding “use” or “method” items. For clarity, for example, a item “A PFKFB3 inhibitor for use in treatment of accelerated aging” for a purpose of this item will give a new item “A kit, comprising PFKFB3 inhibitor and instructions for in treatment of accelerated aging”.

1756. A kit, comprising a medication and instructions or information regarding to degradation, deleting, reducing, binding or inhibiting PFKFB3 for a use, wherein use is selected from any one of preceding “use” or “method” items.

1757. A kit of any one of preceding items, wherein PFKFB3 inhibitor is a compound selected from the compounds described or referenced to in this application.

1758. A kit of any one of preceding items, comprising a composition, which comprises said PFKFB3 inhibitor.

1759. A kit of any one of preceding items, wherein use comprises administering of PFKFB3 inhibitor.

1760. A kit of any one of preceding items, wherein instead of PFKFB3 inhibitor a modulator of Indirect Target is used, wherein a modulation of Indirect Target has anti-aging or neuroprotective effect.

1761. A kit of preceding item, wherein modulator of Indirect Target mimics or effects the inhibition of PFKFB3.

A Tangible Medium

1762. A tangible medium, configured with instructions that when executed cause a processor to perform a method, the method comprising: attribution to the information about a patient an information about an anti-aging treatment related to silencing, deleting, reducing, binding, inhibiting or degrading of PFKFB3.

1763. A tangible medium, configured with instructions that when executed cause a processor to perform a method, the method comprising: attribution to the information about a patient an information about an anti-aging or neuroprotective treatment related to modulating, silencing, deleting, reducing, binding, inhibiting, activating or degrading of at least one of Indirect Targets.

1764. A tangible medium of any one of preceding items, further comprising attributing to the information about patient before or after or before and after the treatment to information about checking of at least one selected from the group: biological age of the patient, at least one aging biomarker, at least one age related deficit or disease, at least one of rejuvenation marker, marker of frailty, marker of health span or marker of life expectancy.

1765. A tangible medium, configured with instructions that when executed cause a processor to perform a method, the method comprising: attribution to the information about a patient an information about neuroprotection related to deleting, reducing, binding, inhibiting or degrading of PFKFB3.

1766. A tangible medium, configured with instructions that when executed cause a processor to perform a method, the method comprising: attribution to the information about a patient an information about method or use described in any one of preceding items.

1767. A tangible medium, configured with instructions that when executed cause a processor to perform a method, the method comprising: attribution to the information about a patient an information about inhibiting of PFKFB3 by small molecule for anti-aging or neuroprotective treatment.

1768. A tangible medium, configured with instructions that when executed cause a processor to perform a method, the method comprising: attribution to the information about deleting, reducing, binding, inhibiting, silencing or degrading PFKFB3 with the information about anti-aging or neuroprotective treatment.

1769. A tangible medium of any one of the preceding items, wherein tangible medium is a machine readable medium.

1770. A tangible medium of any one of the preceding items, wherein tangible medium is a computer.

1771. A tangible medium of any of preceding items, comprising a computer program, which, when executed, causes a device to perform a said attribution.

Common

1772. Kit, method, composition, pharmaceutical composition, use, PFKFB3 inhibitor, medium of any one of preceding items, wherein PFKFB3 inhibitor is PFKFB3 silencing therapy.

1773. Kit, method, composition, pharmaceutical composition, use, PFKFB3 inhibitor, medium of any one of preceding items, wherein PFKFB3 inhibitor is a compound with a specificity for a PFKFB3 polynucleotide selected from the list consisting of an artificial microRNA, ribozyme, an antisense polynucleotide or a small interfering RNA (siRNA).

1774. Kit, method, composition, pharmaceutical composition, use, PFKFB3 inhibitor, medium of any one of preceding items, wherein PFKFB3 inhibitor is a small interfering RNA, wherein said siRNA is produced by an expression construct incorporated into a viral vector

1775. Kit, method, composition, pharmaceutical composition, use, PFKFB3 inhibitor, medium of any one of preceding items, wherein PFKFB3 inhibitor is a small interfering RNA, wherein said siRNA is produced by an expression construct incorporated into an adenoviral-associated viral vector.

1776. Kit, method, composition, pharmaceutical composition, use, PFKFB3 inhibitor, medium of any one of preceding items, wherein PFKFB3 inhibitor is an antisense nucleic acid.

1777. Kit, method, composition, pharmaceutical composition, use, PFKFB3 inhibitor, medium of any one of preceding items, wherein PFKFB3 inhibitor is an antisense nucleic acid selected from the list consisting of a siRNA, a double stranded RNA, a short hairpin RNA.

1778. Kit, method, composition, pharmaceutical composition, use, PFKFB3 inhibitor, medium of any one of preceding items, wherein the subject is a human.

1779. Kit, method, composition, pharmaceutical composition, use, PFKFB3 inhibitor, medium of any one of preceding items, wherein the subject is a human of an age >40 years.

1780. Kit, method, composition, pharmaceutical composition, use, PFKFB3 inhibitor, medium of any one of preceding items, wherein the subject is a human of an age >50 years.

1781. Kit, method, composition, pharmaceutical composition, use, PFKFB3 inhibitor, medium of any one of preceding items, wherein the subject is a human of an age >60 years.

1782. Kit, method, composition, pharmaceutical composition, use, PFKFB3 inhibitor, medium of any one of preceding items, wherein the subject is a human of a biological age >40 years.

1783. Kit, method, composition, pharmaceutical composition, use, PFKFB3 inhibitor, medium of any one of preceding items, wherein the subject is a human of a biological age >50 years.

1784. Kit, method, composition, pharmaceutical composition, use, PFKFB3 inhibitor, medium of any one of preceding items, wherein the subject is a human of a biological age >60 years.

1785. Kit, method, composition, pharmaceutical composition, use, PFKFB3 inhibitor, medium of any one of preceding items, wherein subject is an elderly human.

1786. Kit, method, composition, pharmaceutical composition, use, PFKFB3 inhibitor, medium of any one of preceding items, wherein subject is a male.

1787. Kit, method, composition, pharmaceutical composition, use, PFKFB3 inhibitor, medium of any one of preceding items, wherein subject is a female.

1788. Kit, method, composition, pharmaceutical composition, use, PFKFB3 inhibitor, medium of any one of preceding items, wherein subject suffers from an age-related disease or disorder.

1789. Kit, method, composition, pharmaceutical composition, use, PFKFB3 inhibitor, medium of any one of preceding items wherein said age-related disease or disorder is selected from the group: frailty, Alzheimer's, Parkinson's, and Huntington's diseases, cardiovascular disease, renal failure, muscle wasting [cachexia], osteopenia or osteoporosis, obesity, insulin resistance or diabetes, atherosclerosis, cardiovascular disease, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence (including but not limited to age related decline in immune response to vaccines, age related decline in response to immunotherapy etc.), myocardial infarction, acute coronary syndrome, sarcopenia, sarcopenic obesity, senile osteoporosis, urinary incontinence or any other age related disease, including but not limited to those described in this application or in the sources referenced in this application.

1790. A Kit, method, composition, pharmaceutical composition, use, PFKFB3 inhibitor, medium of any one of preceding items wherein age-related disease or disorder is selected from sarcopenia, menopausal syndrome, atherosclerosis, cardiovascular disease, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence (including but not limited to age related decline in immune response to vaccines, age related decline in response to immunotherapy etc.), myocardial infarction, acute coronary syndrome, sarcopenia, sarcopenic obesity, senile osteoporosis, urinary incontinence, neuromuscular disorder, osteoarthritis, chronic fatigue syndrome, senile dementia, mild cognitive impairment due to aging, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, Lewy body disease, amyotrophic lateral sclerosis (ALS), mild cognitive impairment, pre-dementia, dementia, progressive subcortical gliosis, progressive supranuclear palsy, thalamic degeneration syndrome, hereditary aphasia, myoclonus epilepsy, macular degeneration, frailty, pressure ulcers, delirium or any other age related disease, including but not limited to those described in this application or in the sources referenced in this application.

1791. Kit, method, composition, pharmaceutical composition, use, PFKFB3 inhibitor, medium of any one of preceding items, for use in reducing morbidity or mortality risks in said subject.

1792. Kit, composition, pharmaceutical composition, PFKFB3 inhibitor, medium of any one of preceding items or described in this application, for use selected from the group: in treatment leading to prevention, amelioration or lessening the effects of aging, decreasing or delaying an increase in the biological age, slowing rate of aging; treatment, prevention, amelioration and lessening the effects of frailty or at least one of aging related diseases and conditions or declines or slowing down the progression of such decline, condition or disease, increasing health span, increasing lifespan, rejuvenation, increasing stress resistance or resilience, increasing rate or other enhancement of recovery after surgery, radiotherapy, disease and/or any other stress, prevention and/or the treatment of menopausal syndrome, restoring reproductive function, eliminating or decrease in spreading of senescent cells, decreasing all-causes or multiple causes of mortality risks or mortality risks related to at least one or at least two of age related diseases or conditions or delaying in increase of such risks, decreasing morbidity risks, modulating at least one of biomarkers of aging into more youthful state or slowing down its change into “elder” state, including but not limited to biomarkers of aging which are visible signs of aging, such as wrinkles, grey hairs etc.

1793. Kit, method, composition, pharmaceutical composition, use, PFKFB3 inhibitor, medium of any one of preceding items, wherein the subject suffers from a disease or a condition or decline selected from those described in anti-aging treatment definition in this application.

1794. Kit, method, composition, pharmaceutical composition, use, PFKFB3 inhibitor, medium of any one of preceding items, wherein PFKFB3 inhibitor is selected from described in this application or is its structural or functional analog.

1795. Kit, method, composition, pharmaceutical composition, use, PFKFB3 inhibitor, medium of any one of preceding items, wherein instead of PFKFB3 inhibitor described in such item, an agent comprising a PFKFB3 inhibitor or other molecule described in this application or its structural or functional analog of it is used.

1796. Kit, method, composition, pharmaceutical composition, use, PFKFB3 inhibitor, medium of any one of preceding items or described in this description, wherein PFKFB3 inhibitor or other agent mentioned there is a pharmaceutically acceptable salt of such inhibitor or such other agent.

1797. Kit, method, composition, pharmaceutical composition, use, PFKFB3 inhibitor, medium of any one of preceding items or described in this description, wherein PFKFB3 inhibitor or other agent mentioned there is a pharmaceutically acceptable acid addition salt of such inhibitor or such other agent, or a hydrate or solvate thereof.

1798. Kit, method, composition, pharmaceutical composition, use, PFKFB3 inhibitor, medium of any one of preceding items or described in this description, wherein PFKFB3 inhibitor or other agent mentioned there is a pharmaceutically acceptable acid addition salt of such inhibitor or such other agent, or a hydrate or solvate thereof, wherein the pharmaceutically acceptable acid addition salts thereof selected from the salts obtained with hydrochloric acid, hydrobromic acid, sulphuric acid, phosphoric acid, methanesulphonic acid, acetic acid, fumaric acid, succinic acid, lactic acid, citric acid, tartaric acid and maleic acid.

1799. Kit, method, composition, pharmaceutical composition, use, PFKFB3 inhibitor, medium of any one of preceding items or described in this description, wherein PFKFB3 inhibitor or other agent mentioned there is in a therapeutically effective amount

1800. Kit, method, composition, pharmaceutical composition, use, PFKFB3 inhibitor, medium of any one of preceding items or described in this description, wherein PFKFB3 inhibitor or other agent mentioned there is administered intravenously.

1801. Kit, method, composition, pharmaceutical composition, use, PFKFB3 inhibitor, medium of any one of preceding items or described in this description, wherein PFKFB3 inhibitor or other agent mentioned there is administered perorally

1802. Kit, method, composition, pharmaceutical composition, use, PFKFB3 inhibitor, medium of any one of preceding items or described in this description, wherein PFKFB3 inhibitor or other agent mentioned there is administered by the route selected from those described in this application.

1803. Kit, method, composition, pharmaceutical composition, use, PFKFB3 inhibitor, medium of any one of preceding items or described in this description, wherein PFKFB3 inhibitor, modulator or degradation agent is a peptide, small molecule, antibody, aptamer, protein, virus, polymer, nanoparticle or particle.

1804. Kit, method, composition, pharmaceutical composition, use, PFKFB3 inhibitor, medium of any one of preceding items or described in this description, wherein PFKFB3 inhibitor is a gene therapy.

1805. Kit, method, composition, pharmaceutical composition, use, PFKFB3 inhibitor, medium of any one of preceding items, wherein PFKFB3 inhibitor is any one of small molecule PFKFB3 inhibitors.

1806. Kit, method, composition, pharmaceutical composition, use, PFKFB3 inhibitor, medium of any one of preceding items, wherein instead of PFKFB3 inhibitor a modulator of Indirect Target is used.

1807. Kit, method, composition, pharmaceutical composition, use, PFKFB3 inhibitor, medium of any one of preceding items, wherein modulation of Indirect Target mimics or effects the reduction or inhibition of PFKFB3.

1808. A PFKFB3 inhibitor for use in manufacturing of therapy for a use or method of any one of the preceding items. For clarity, for example, a item “A PFKFB3 inhibitor for use in treatment of accelerated aging” for a purpose of this item will give a new item “A PFKFB3 inhibitor for use in manufacturing of therapy for treatment of accelerated aging”.

1809. Method of selecting a patient for therapy comprising a PFKFB3 inhibition or PFKFB3 inhibitor, comprising a step of identifying the patient in need of neuroprotection.

1810. Method of selecting a patient for therapy based on PFKFB3 inhibition or PFKFB3 inhibitor, comprising a step of identifying the patient in need of treatment of any one of disease or condition from any one of preceding items.

1811. Any one of the preceding items, wherein instead of method described in such item at least one other method described in this disclosure is used.

1812. Any one of the preceding items, wherein instead of kit described in such item at least one other kit described in this disclosure is used.

1813. Kit, method, composition, pharmaceutical composition, use, PFKFB3 inhibitor, medium of any one of preceding items, wherein a PFKFB3 inhibitor is a small molecule.

1814. Kit, method, composition, pharmaceutical composition, use, PFKFB3 inhibitor, medium of any one of preceding items, wherein instead the wording “a PFKFB3 inhibitor” a word “compound” is used.

1815. A PFKFB3 inhibitor of one of preceding items, wherein a PFKFB3 inhibitor is a small molecule inhibitor of kinase activity of PFKFB3.

1816. A method of manufacturing a medication, comprising the compound of any one of items 1-199 for use as an active ingredient, wherein the medicament is for at least one of the uses or methods of any one of the preceding items.

1817. The compound of any one of items 1-199 for use for manufacturing a medicament.

1818. A kit for treating a disease of any one of preceding items, comprising (a) a pharmaceutical composition comprising a compound of any one of items 1 to 199; and (b) instructions for use.

1819. A kit, comprising (a) a pharmaceutical composition comprising a compound of any one of items 1 to 199; and (b) instructions for use.

1820. A kit, comprising (a) a pharmaceutical composition comprising a compound of any one of items 1 to 199; and (b) instructions for administration of such composition.

1821. A method of neuroprotection, comprising administering of PFKFB3 inhibitor, wherein PFKFB3 inhibitor is selected from any one of the items 1 to 199.

1822. A method of neuroprotection, comprising administering of PFKFB3 inhibitor, wherein PFKFB3 inhibitor is selected from any one of the items 338 to 1545 or items A,B,C,D,E, F, G or H.

1823. An invention of any one preceding items, wherein compound mentioned in such item is comprised in composition, further comprising at least one of pharmaceutically acceptable excipients.

1824. A compound for use as neuroprotector, wherein compound is selected from any one of the items 1 to 199.

1825. A compound for use as anti-aging treatment, wherein compound is selected from any one of the items 1 to 199.

1826. A compound for use, wherein use is selected from any one of the preceding items, wherein PFKFB3 inhibitor is selected from any one of the items 1 to 199.

1827. A PFKFB3 inhibitor for use as neuroprotector, wherein PFKFB3 inhibitor is selected from any one of the items 338 to 1545 or items A,B,C,D,E,F, G, H (wording equal to A-H).

1828. PFKFB3 inhibitor for use as anti-aging treatment, wherein PFKFB3 inhibitor is selected from any one of the items 338 to 1545 or items A,B,C,D,E,F,G, H.

1829. A method of neuroprotection, comprising administering of PFKFB3 inhibitor, wherein PFKFB3 inhibitor is selected from any one of the items 1 to 199.

1830. A method of neuroprotection, comprising administering of PFKFB3 inhibitor, wherein PFKFB3 inhibitor is selected from any one of the items 338 to 1545 or items A,B,C,D,E, F,G,H.

1831. Method of anti-aging treatment, comprising administering of compound, wherein compound is selected from any one of the items 1 to 199.

1832. Method of any one of preceding items, wherein PFKFB3 inhibitor is selected from any one of the items 1 to 199.

1833. Method of anti-aging treatment, comprising administering of PFKFB3 inhibitor, wherein PFKFB3 inhibitor is selected from any one of the items 338 to 1545 or items A,B,C,D,E, F,G, H.

1834. Kit, method, composition, pharmaceutical composition, use, PFKFB3 inhibitor, medium of any one of preceding items or described in this description, wherein PFKFB3 inhibitor is selected from any one of the items 1 to 199.

1835. Kit, method, composition, pharmaceutical composition, use, PFKFB3 inhibitor, medium of any one of preceding items or described in this description, wherein PFKFB3 inhibitor is selected from any one of the items of the items 338 to 1545 or items A,B,C,D,E, F,G,H.

1836. Kit, method, composition, pharmaceutical composition, use, PFKFB3 inhibitor, medium of any one of preceding items or described in this description, wherein PFKFB3 inhibitor or other agent mentioned there is a pharmaceutically acceptable pharmaceutically acid addition salt of such inhibitor or such other agent, or a hydrate or solvate thereof, wherein the pharmaceutically acceptable acid addition salts thereof selected from the salts obtained with hydrochloric acid, hydrobromic acid, sulphuric acid, phosphoric acid, methanesulphonic acid, acetic acid, fumaric acid, succinic acid, lactic acid, citric acid, tartaric acid and maleic acid.

1837. Kit, method, composition, pharmaceutical composition, use, PFKFB3 inhibitor, medium of any one of preceding items, wherein PFKFB3 inhibitor is a structural analog, functional analog, derivative, N-oxide, prodrug, solvate, tautomer, stereoisomer, racemate, physiologically acceptable salt, including mixtures thereof in all ratios of PFKFB3 inhibitor selected from of any one of preceding items.

1838. Kit, method, composition, pharmaceutical composition, use, PFKFB3 inhibitor, medium of any one of preceding items or described in this description, wherein PFKFB3 inhibitor or other agent mentioned there is thereof is in the form of a prodrug.

1839. Kit, method, composition, pharmaceutical composition, use, PFKFB3 inhibitor, medium of any one of preceding items or described in this description, wherein PFKFB3 inhibitor or other agent mentioned there is thereof is in the form of a prodrug, wherein the prodrug comprises an ester moiety.

1840. Kit, method, composition, pharmaceutical composition, use, PFKFB3 inhibitor, medium of any one of preceding items or described in this description, wherein PFKFB3 inhibitor or other agent mentioned there is thereof is in the form of a prodrug, wherein the prodrug comprises an amide moiety.

1841. PFKFB3 inhibitor for use in enhancement of T-cell function for adoptive T-cell therapy, wherein PFKFB3 inhibitor is selected from any one of the claims 1 to 199 or any one of the claims 254 to 264 or any one of the items 338 to 1545 or any one of the items A,B,C,D,E, F, G or H.

1842. Method of enhancement of T-cell function for adoptive T-cell therapy, comprising administering PFKFB3 inhibitor in therapeutically effective amount.

1843. Method of enhancement of T-cell function for adoptive T-cell therapy, comprising administering PFKFB3 inhibitor in therapeutically effective amount, wherein PFKFB3 inhibitor is selected from any one of the claims 1 to 199 or any one of the claims 254 to 264 or any one of the items 338 to 1545 or any one of the items A,B,C,D,E, F, G or H.

1844. PFKFB3 inhibitor for use in treatment of reperfusion injury (or reperfusion insult or reoxygenation injury), wherein PFKFB3 inhibitor is selected from any one of the claims 1 to 199 or any one of the claims 254 to 264 or any one of the items 338 to 1545 or any one of the items A,B,C,D,E, F, G or H.

1845. PFKFB3 inhibitor for use in prevention of reperfusion injury (or reperfusion insult or reoxygenation injury), wherein PFKFB3 inhibitor is selected from any one of the claims 1 to 199 or any one of the claims 254 to 264 or any one of the items 338 to 1545 or any one of the items A,B,C,D,E, F, G or H.

1846. Method of treatment of reperfusion injury (or reperfusion insult or reoxygenation injury), comprising administering PFKFB3 inhibitor in therapeutically effective amount, wherein PFKFB3 inhibitor is selected from any one of the claims 1 to 199 or any one of the claims 254 to 264 or any one of the items 338 to 1545 or any one of the items A,B,C,D,E, F, G or H.

1847. Method of prevention of reperfusion injury (or reperfusion insult or reoxygenation injury), comprising administering PFKFB3 inhibitor in therapeutically effective amount, wherein PFKFB3 inhibitor is selected from any one of the claims 1 to 199 or any one of the claims 254 to 264 or any one of the items 338 to 1545 or any one of the items A,B,C,D,E, F, G or H.

1848. Method of prevention of retinopathy comprising administering PFKFB3 inhibitor in therapeutically effective amount, wherein PFKFB3 inhibitor is selected from any one of the claims 1 to 199.

1849. Method of treatment of retinopathy comprising administering PFKFB3 inhibitor in therapeutically effective amount, wherein PFKFB3 inhibitor is selected from any one of the claims 1 to 199.

1850. PFKFB3 inhibitor for use in prevention of retinopathy, wherein PFKFB3 inhibitor is selected from any one of the claims 1 to 199.

1851. PFKFB3 inhibitor for use in treatment of retinopathy, wherein PFKFB3 inhibitor is selected from any one of the claims 1 to 199.

1852. Method of treatment of brain tumors, comprising administering PFKFB3 inhibitor in therapeutically effective amount, wherein PFKFB3 inhibitor is selected from any one of the claims 1 to 199.

1853. Method of treatment of CNS primitive neuroectodermal tumors, comprising administering PFKFB3 inhibitor in therapeutically effective amount, wherein PFKFB3 inhibitor is selected from any one of the claims 1 to 199.

1854. PFKFB3 inhibitor for use in treatment of brain tumors, wherein PFKFB3 inhibitor is selected from any one of the claims 1 to 199.

1855. PFKFB3 inhibitor for use in treatment of CNS primitive neuroectodermal tumors, wherein PFKFB3 inhibitor is selected from any one of the claims 1 to 199.

1856. A pharmaceutical composition comprising a compound of any one of items 1-1545 in combination with another therapeutic agent, and optionally, one or more pharmaceutically acceptable carriers.

1857. The pharmaceutical composition of preceding item, further comprising a second therapeutic agent.

1858. Kit, method, composition, pharmaceutical composition, use, PFKFB3 inhibitor, medium of any one of preceding items or described in this description, wherein PFKFB3 inhibitor or other agent mentioned there is in a therapeutically effective amount.

1859. PFKFB3 inhibitor for use in neuroprotection or described in this description, wherein PFKFB3 inhibitor or other agent mentioned there is a pharmaceutically acceptable pharmaceutically salt of such inhibitor or such other agent, or a hydrate or solvate thereof, wherein the pharmaceutically acceptable acid addition salts thereof selected from the salts obtained with hydrochloric acid, hydrobromic acid, sulphuric acid, phosphoric acid, methanesulphonic acid, acetic acid, fumaric acid, succinic acid, lactic acid, citric acid, tartaric acid and maleic acid.

1860. PFKFB3 inhibitor for use in neuroprotection, wherein PFKFB3 inhibitor is a structural analog, functional analog, derivative, N-oxide, prodrug, solvate, tautomer, stereoisomer, racemate, physiologically acceptable salt, including mixtures thereof in all ratios of PFKFB3 inhibitor selected from of any one of preceding items.

1861. PFKFB3 inhibitor for use in neuroprotection or described in this description, wherein PFKFB3 inhibitor or other agent mentioned there is in a therapeutically effective amount.

1862. Any one of the preceding items, wherein instead of words “PFKFB3 inhibitor” a word “compound” is used.

1863. Any one of the preceding items, wherein instead of words “of any one of the preceding items” the words “of any one of the items 1 to 1862” are used.

### EXAMPLES

### Example I: In Vitro Activity in PFKFB3 and PFKFB4 Assay

Recombinant full length human PFKFB3 (or PFKFB4) protein purified from Sf9 baculoviral system was acquired from SignalChem (Cat. #P323-30G or P324-30G). ATP, Fructose-6-Phosphate and other chemicals were from Sigma-Aldrich. The kinase activity of the PFKFB3 (PFKFB4) protein was detected by measuring production of ADP from ATP in the presence of the Fructose-6-Phosphate substrate. The kinase reactions were assembled in 384 well plates in a total volume of 25 μL. Test compounds were serially diluted in DMSO. Reactions were set up by first mixing test compounds with enzyme and pre-incubating for 15 min. The ATP and Fructose-6-Phosphate substrates were next added to initiate the kinase reactions. The final assay composition included 100 mM Tris-HCl pH8.0, 4 mM MgCl₂, 5 mM KH₂PO₄, 5 mM dithiothreitol (DTT), 20 mM KF, 0.02% BSA, 10 nM enzyme, 20 μM ATP (Km=16 μM), 10 μM F₆P (Km=6 μM).

The compounds of invention listed below in this example were tested at various concentrations (which also added 1% DMSO from compounds, to the final solution) as described below. The kinase reactions were allowed to proceed for 1 h at room temperature. Aliquots of the reaction mixtures (5 μL) were transferred to fresh white 384 well plates and mixed with 5 μL of the ADP-Glo reagent (Promega), followed by incubation for 30 min. The luminescent Kinase Detection reagent was added (10 μL) and, following additional incubation for 15 min, the plates were read on luminescence plate reader (Analyst HT). Positive (100%-inhibition) and negative (0%-inhibition) control samples were assembled in each assay plate and were used to calculate percent inhibition values of test compounds. All experiments were performed in duplicate. PFKFB3 and PFKFB4 inhibition data are shown in Table 2. All compounds of this invention can be tested in the same assay and will show at least moderate activity on PFKFB3 and/or PFKFB4, that is a prophetic example of their activity towards PFKFB3 and/or PFKFB4 in case the data from the conducted experiments are not provided herein.

A<0.5 μM; 0.5 μM≤B<2 μM; 2 μM≤C<5 μM; 5 μM≤D<20 μM; E≤20 μM; ND=Not determined

| | | IC₅₀PFKFB3 |
|---|---|---|
| Ex. | Name | (PFKFB4*) |
| 1 | 2-(2-Methoxybiphenyl-4-yl)-1,3-dioxo-2,3-dihydro-1H-isoindole-5-carboxylic acid | D |
| 2 | 1,3-Dioxo-2-[3-(1H-tetrazol-5-yl)-1,1′-biphenyl-4-yl]-2,3-dihydro-1H-isoindole-5-carboxylic | A |
| | acid | |
| 3 | 2-(4-Hydroxy[1,1′-biphenyl]-3-yl)-1,3-dioxo-2,3-dihydro-1H-isoindole-5-carboxylic acid | B |
| 4 | 2-(3-Hydroxymethylbiphenyl-4-yl)-1,3-dioxo-2,3-dihydro-1H-isoindole-5-carboxylic acid | C |
| 5 | 2-(3-Methoxycarbonylbiphenyl-4-yl)-1,3-dioxo-2,3-dihydro-1H-isoindole-5-carboxylic acid | ND |
| | methyl ester | |
| 6 | 2-[3-Cyano-4-(4-methoxyphenyl)-5-methylthiophen-2-yl]-1,3-dioxo-2,3-dihydro-1H- | A |
| | isoindole-5-carboxylic acid | |
| 7 | 2-[3-Cyano-4-(4-methoxyphenyl)-5-methylthiophen-2-yl]-1,3-dioxo-2,3-dihydro-1H- | ND |
| | isoindole-5-carboxylic acid ethyl ester | |
| 8 | 2-[3-Cyano-4-(4-methoxyphenyl)-5-methylthiophen-2-yl]-1,3-dioxo-2,3-dihydro-1H- | ND |
| | isoindole-5-carboxylic acid butyl ester | |
| 9 | 1,3-Dioxo-2-[3-(1H-tetrazol-5-yl)biphenyl-4-yl]-2,3-dihydro-1H-isoindole-5-carboxylic acid | ND |
| | ethyl ester | |
| 10 | 2-(4-Carboxybiphenyl-3-yl)-1,3-dioxo-2,3-dihydro-1H-isoindole-5-carboxylic acid | A (A) |
| 11 | 2-(4-Hydroxymethylbiphenyl-3-yl)-1,3-dioxo-2,3-dihydro-1H-isoindole-5-carboxylic acid | D |
| 12 | 2-(3-Methoxycarbonylbiphenyl-4-yl)-1,3-dioxo-2,3-dihydro-1H-isoindole-5-carboxylic acid | C |
| 13 | N-{2-[3-Cyano-4-(4-methoxyphenyl)-5-methylthiophen-2-yl]-1,3-dioxo-2,3-dihydro-1H- | D |
| | isoindole-5-carbonyl}-benzenesulfonamide | |
| 14 | N-{2-[3-Cyano-4-(4-methoxyphenyl)-5-methylthiophen-2-yl]-1,3-dioxo-2,3-dihydro-1H- | B |
| | isoindole-5-carbonyl}-methanesulfonamide | |
| 15 | Butane-1-sulfonic acid {2-[3-cyano-4-(4-methoxyphenyl)-5-methylthiophen-2-yl]-1,3-dioxo- | D |
| | 2,3-dihydro-1H-isoindole-5-carbonyl}-amide | |
| 16 | (2S)-2-{[2-(3-cyano-4-(4-methoxyphenyl)-5-methylthiophen-2-yl)-1,3-dioxo-2,3-dihydro-1H- | C |
| | isoindole-5-carbonyl]amino}-3-hydroxypropanoic acid | |
| 17 | (2S)-2-({2-[3-Cyano-4-(4-methoxyphenyl)-5-methylthiophen-2-yl]-1,3-dioxo-2,3-dihydro- | B |
| | 1H-isoindole-5-carbonyl}amino)-3-(1H-indol-3-yl)propionic acid | |
| 18 | (2S)-2-({2-[3-Cyano-4-(4-methoxyphenyl)-5-methylthiophen-2-yl]-1,3-dioxo-2,3-dihydro- | D |
| | 1H-isoindole-5-carbonyl}-amino)-4-methyl-pentanoic acid | |
| 19 | (2S)-2-({2-[3-Cyano-4-(4-methoxyphenyl)-5-methyl-thiophen-2-yl]-1,3-dioxo-2,3-dihydro- | D |
| | 1H-isoindole-5-carbonyl}-amino)-3-methyl-butyric acid | |
| 20 | (2S)-2-({2-[3-Cyano-4-(4-methoxyphenyl)-5-methyl-thiophen-2-yl]-1,3-dioxo-2,3-dihydro- | D |
| | 1H-isoindole-5-carbonyl}-amino)-succinamic acid | |
| 21 | (2S)-4-Carbamoyl-2-({2-[3-cyano-4-(4-methoxyphenyl)-5-methyl-thiophen-2-yl]-1,3-dioxo- | D |
| | 2,3-dihydro-1H-isoindole-5-carbonyl}-amino)-butyric acid | |
| 22 | 4-(5-Benzenesulfonylaminocarbonyl-1,3-dioxo-1,3-dihydroisoindol-2-yl)biphenyl-3- | C |
| | carboxylic acid | |
| 23 | 4-(5-Methanesulfonylaminocarbonyl-1,3-dioxo-1,3-dihydroisoindol-2-yl)biphenyl-3- | B |
| | carboxylic acid | |
| 24 | 4-[5-(Butane-1-sulfonylaminocarbonyl)-1,3-dioxo-1,3-dihydroisoindol-2-yl]biphenyl-3- | C |
| | carboxylic acid | |
| 25 | 3-(5-Methanesulfonylaminocarbonyl-1,3-dioxo-1,3-dihydroisoindol-2-yl)biphenyl-4- | A |
| | carboxylic acid | |
| 26 | 3-[5-(Butane-1-sulfonylaminocarbonyl)-1,3-dioxo-1,3-dihydroisoindol-2-yl]biphenyl-4- | C |
| | carboxylic acid | |
| 27 | 4-(5-Carbamoyl-1,3-dioxo-1,3-dihydroisoindol-2-yl)biphenyl-3-carboxylic acid | C |
| 28 | 3-(5-Carbamoyl-1,3-dioxo-1,3-dihydroisoindol-2-yl)biphenyl-4-carboxylic acid | B |
| 29 | 4-[5-(1-Benzyl-1H-[1,2,3]triazol-4-yl)-1,3-dioxo-1,3-dihydroisoindol-2-yl]biphenyl-3- | C |
| | carboxylic acid | |
| 30 | 4-{5-[1-(2,2-Dimethylpropionyloxymethyl)-1H-[1,2,3]triazol-4-yl]-1,3-dioxo-1,3- | C |
| | dihydroisoindol-2-yl}biphenyl-3-carboxylic acid | |
| 31 | 4-[1,3-Dioxo-5-(1H-[1,2,3]triazol-4-yl)-1,3-dihydroisoindol-2-yl]biphenyl-3-carboxylic acid | A |
| 32 | 2-[1,3-Dioxo-5-(1H-[1,2,3]triazol-4-yl)-1,3-dihydroisoindol-2-yl]-4-(4-methoxyphenyl)-5- | C |
| | methylthiophene-3-carbonitrile | |
| 33 | 4-[1,3-Dioxo-5-(1H-[1,2,3]triazol-4-yl)-1,3-dihydroisoindol-2-yl]biphenyl-3-carboxylic acid | E |
| | methyl ester | |
| 34 | 3-[1,3-Dioxo-5-(1H-[1,2,3]triazol-4-yl)-1,3-dihydroisoindol-2-yl]biphenyl-4-carboxylic acid | D |
| | methyl ester | |
| 35 | 2-(3-Methoxycarbonylbiphenyl-4-yl)-1,3-dioxo-2,3-dihydro-1H-isoindole-5-carboxylic acid | E |
| | butyl ester | |
| 36 | 2-(4-Methoxycarbonylbiphenyl-3-yl)-1,3-dioxo-2,3-dihydro-1H-isoindole-5-carboxylic acid | E |
| | butyl ester | |
| 37 | 2-(4-Methoxycarbonylbiphenyl-3-yl)-1,3-dioxo-2,3-dihydro-1H-isoindole-5-carboxylic acid | B |
| 38 | 4-[1,3-Dioxo-5-(1H-[1,2,3]triazol-4-yl)-1,3-dihydroisoindol-2-yl]-6-methoxybiphenyl-3- | D |
| | carboxylic acid methyl ester | |
| 39 | 2-(2-Methoxy-5-methoxycarbonylbiphenyl-4-yl)-1,3-dioxo-2,3-dihydro-1H-isoindole-5- | E |
| | carboxylic acid butyl ester | |
| 40 | 4-(5-Methanesulfonylaminocarbonyl-1,3-dioxo-1,3-dihydroisoindol-2-yl)biphenyl-3- | E |
| | carboxylic acid methyl ester | |
| 41 | 3-(5-Methanesulfonylaminocarbonyl-1,3-dioxo-1,3-dihydroisoindol-2-yl)biphenyl-4- | E |
| | carboxylic acid methyl ester | |
| 42 | 2-(6-Methoxy-3-methoxycarbonylbiphenyl-4-yl)-1,3-dioxo-2,3-dihydro-1H-isoindole-5- | B |
| | carboxylic acid | |
| 43 | 1,3-Dioxo-2-[4-(1H-tetrazol-5-yl)biphenyl-3-yl]-2,3-dihydro-1H-isoindole-5-carboxylic acid | B |
| 44 | 3-[1,3-Dioxo-5-(1H-tetrazol-5-yl)-1,3-dihydroisoindol-2-yl]biphenyl-4-carboxylic acid methyl | C |
| | ester | |
| 45 | 2-(4-Carboxy-4′-fluorobiphenyl-3-yl)-1,3-dioxo-2,3-dihydro-1H-isoindole-5-carboxylic acid | A |
| 46 | 2-(4-Carboxy-3′-fluorobiphenyl-3-yl)-1,3-dioxo-2,3-dihydro-1H-isoindole-5-carboxylic acid | A |
| 47 | 2-[5-Methoxy-2-(1H-tetrazol-5-yl)phenyl]-1,3-dioxo-2,3-dihydro-1H-isoindole-5-carboxylic | B |
| | acid | |
| 48 | 2-(2-Carboxy-5-thiophen-2-yl-phenyl)-1,3-dioxo-2,3-dihydro-1H-isoindole-5-carboxylic acid | A |
| 49 | 2-[1,3-Dioxo-5-(1H-[1,2,3]triazol-4-yl)-1,3-dihydroisoindol-2-yl]-4-pyridin-3-yl-benzoic acid | C |
| 50 | 3-[1,3-Dioxo-5-(1H-[1,2,3]triazol-4-yl)-1,3-dihydroisoindol-2-yl]-3′-fluorobiphenyl-4- | B |
| | carboxylic acid | |
| 51 | 3-[1,3-Dioxo-5-(1H-[1,2,3]triazol-4-yl)-1,3-dihydroisoindol-2-yl]-2′,4′-difluorobiphenyl-4- | A |
| | carboxylic acid | |
| 52 | 2-(2-Carboxy-5-pyridin-3-yl-phenyl)-1,3-dioxo-2,3-dihydro-1H-isoindole-5-carboxylic acid | A |
| 53 | 2-(4-Carboxy-2′,4′-difluorobiphenyl-3-yl)-1,3-dioxo-2,3-dihydro-1H-isoindole-5-carboxylic | A |
| | acid | |
| 54 | 2-[4-(1H-Tetrazol-5-yl)biphenyl-3-yl]-5-(1H-[1,2,3]triazol-4-yl)-isoindole-1,3-dione | C |
| 55 | 2-(4-Carboxy-4′-methoxybiphenyl-3-yl)-1,3-dioxo-2,3-dihydro-1H-isoindole-5-carboxylic | A |
| | acid | |
| 56 | 2-[1,3-Dioxo-5-(1H-[1,2,3]triazol-4-yl)-1,3-dihydroisoindol-2-yl]-4-thiophen-2-yl-benzoic | C |
| | acid | |
| 57 | 3-[1,3-Dioxo-5-(1H-[1,2,3]triazol-4-yl)-1,3-dihydroisoindol-2-yl]-4′-fluorobiphenyl-4- | C |
| | carboxylic acid | |
| 58 | 3-[1,3-Dioxo-5-(1H-[1,2,3]triazol-4-yl)-1,3-dihydroisoindol-2-yl]-4′-methoxybiphenyl-4- | B |
| | carboxylic acid | |
| 59 | 3-[1,3-Dioxo-5-(1H-[1,2,3]triazol-4-yl)-1,3-dihydroisoindol-2-yl]biphenyl-4-carboxylic acid | A |
| 60 | 2-[1,3-Dioxo-5-(1H-[1,2,3]triazol-4-yl)-1,3-dihydroisomdol-2-yl]-5-(3H-imidazol-4-yl)- | A |
| | benzoic acid | |
| 61 | 2-[1,3-Dioxo-5-(1H-[1,2,3]triazol-4-yl)-1,3-dihydroisoindol-2-yl]-5-(3-methyl-3H-imidazol- | D |
| | 4-yl)-benzoic acid | |
| 62 | 2-[1,3-Dioxo-5-(1H-[1,2,3]triazol-4-yl)-1,3-dihydroisoindol-2-yl]-5-pyridin-3-yl-benzoic acid | C |
| 63 | 2-[1,3-Dioxo-5-(1H-[1,2,3]triazol-4-yl)-1,3-dihydroisoindol-2-yl]-5-pyrazin-2-yl-benzoic acid | D |
| 64 | 2-[1,3-Dioxo-5-(1H-[1,2,3]triazol-4-yl)-1,3-dihydroisoindol-2-yl]-5-pyrimidin-5-yl-benzoic | E |
| | acid | |
| 65 | 5-(6-Amino-pyridin-3-yl)-2-[1,3-dioxo-5-(1H-[1,2,3]triazol-4-yl)-1,3-dihydroisoindol-2-yl]- | C |
| | benzoic acid | |
| 66 | 3-[1,3-Dioxo-5-(1H-[1,2,3]triazol-4-yl)-1,3-dihydroisoindol-2-yl]biphenyl-4-carboxylic acid | C |
| | 2-dimethylamino-ethyl ester | |
| 67 | 3-[1,3-Dioxo-5-(1H-[1,2,3]triazol-4-yl)-1,3-dihydroisoindol-2-yl]biphenyl-4-carboxylic acid | D |
| | 2-morpholin-4-yl-ethyl ester | |
| 68 | 3-[1,3-Dioxo-5-(1H-[1,2,3]triazol-4-yl)-1,3-dihydroisoindol-2-yl]biphenyl-4-carboxylic acid | C |
| | 2-pyrrolidin-1-yl-ethyl ester | |
| 69 | 3-[1,3-Dioxo-5-(1H-[1,2,3]triazol-4-yl)-1,3-dihydroisoindol-2-yl]biphenyl-4-carboxylic acid | C |
| | 2-methoxy-ethyl ester | |
| 70 | 3-[1,3-Dioxo-5-(1H-[1,2,3]triazol-4-yl)-1,3-dihydroisoindol-2-yl]biphenyl-4-carboxylic acid | D |
| | 2,3-dihydroxy-propyl ester | |
| 71 | 2-[1,3-Dioxo-5-(1H-[1,2,3]triazol-4-yl)-1,3-dihydroisoindol-2-yl]-5-(1H-pyrazol-4-yl)- | B |
| | benzoic acid | |
| 72 | 3-[1,3-Dioxo-5-(1H-[1,2,3]triazol-4-yl)-1,3-dihydroisoindol-2-yl]biphenyl-4-carboxylic acid | E |
| | 2-amino-ethyl ester | |
| 73 | 3-[1,3-Dioxo-5-(1H-[1,2,3]triazol-4-yl)-1,3-dihydroisoindol-2-yl]biphenyl-4-carboxylic acid | C |
| | (2S)-2-amino-2-carboxy-ethyl ester | |
| 74 | 3-[1,3-Dioxo-5-(1H-[1,2,3]triazol-4-yl)-1,3-dihydroisoindol-2-yl]biphenyl-4-carboxylic acid | E |
| | 2-{[(2S)-2-amino-3-methylbutanoyl]oxy}ethyl ester | |
| 75 | 2-[2-Carboxy-4-(1H-imidazol-4-yl)-phenyl]-1,3-dioxo-2,3-dihydro-1H-isoindole-5-carboxylic | A |
| | acid | |
| 76 | 2-[2-Carboxy-4-(3-methyl-3H-imidazol-4-yl)-phenyl]-1,3-dioxo-2,3-dihydro-1H-isoindole-5- | A |
| | carboxylic acid | |
| 77 | 2-[4-(1H-Imidazol-4-yl)-2-(2-methylaminoethoxycarbonyl)-phenyl]-1,3-dioxo-2,3-dihydro- | B |
| | 1H-isoindole-5-carboxylic acid | |
| 78 | 4-[1,3-Dioxo-5-(1H-[1,2,3]triazol-4-yl)-1,3-dihydroisoindol-2-yl]biphenyl-3-carboxylic acid | D |
| | 2-dimethylamino-ethyl ester | |
| 79 | 4-[1,3-Dioxo-5-(1H-[1,2,3]triazol-4-yl)-1,3-dihydroisoindol-2-yl]biphenyl-3-carboxylic acid | D |
| | 2-pyrrolidin-1-yl-ethyl ester | |
| 80 | 4-[1,3-Dioxo-5-(1H-[1,2,3]triazol-4-yl)-1,3-dihydroisoindol-2-yl]biphenyl-3-carboxylic acid | E |
| | 2-morpholin-4-yl-ethyl ester | |
| 81 | 4-[1,3-Dioxo-5-(1H-[1,2,3]triazol-4-yl)-1,3-dihydroisoindol-2-yl]biphenyl-3-carboxylic acid | E |
| | 2-methylamino-ethyl ester | |
| 82 | 3′,4′-Difluoro-3-[1,3-dioxo-5-(1H-[1,2,3]triazol-4-yl)-1,3-dihydroisoindol-2-yl]biphenyl-4- | A (A) |
| | carboxylic acid | |
| 83 | 2-(2-Hydroxy-5-phenylpyridin-3-yl)-5-(1H-[1,2,3]triazol-4-yl)-isoindole-1,3-dione | C |
| 84 | 2-(4-Phenylpyridin-2-yl)-5-(1H-[1,2,3]triazol-4-yl)-isoindole-1,3-dione | D |
| 85 | 2-{1,3-Dioxo-5-(1H-[1,2,3]triazol-4-yl)-2,3-dihydro-1H-isoindol-2-yl}-4-phenylpyridine N- | D |
| | oxide | |
| 86 | 2-(5-Phenylpyridin-2-yl)-5-(1H-[1,2,3]triazol-4-yl)-isoindole-1,3-dione | E |
| 87 | 2-[4-(4-Fluorophenyl)-pyridin-2-yl]-5-(1H-[1,2,3]triazol-4-yl)-isoindole-1,3-dione | D |
| 88 | 2-(6-Methyl-4-phenylpyridin-2-yl)-5-(1H-[1,2,3]triazol-4-yl)-isoindole-1,3-dione | D |
| 89 | 2-(2-Hydroxy-6-phenylpyridin-3-yl)-5-(1H-[1,2,3]triazol-4-yl)-isoindole-1,3-dione | E |
| 90 | 2-[4-(2,4-Difluoro-phenyl)-5-methyl-pyridin-2-yl]-5-(1H-[1,2,3]triazol-4-yl)-isoindole-1,3- | C |
| | dione | |
| 91 | 3-[1-Oxo-6-(1H-[1,2,3]triazol-4-yl)-1,3-dihydroisoindol-2-yl]biphenyl-4-carboxylic acid | E |
| | methyl ester | |
| 92 | 3-[1-Oxo-6-(1H-[1,2,3]triazol-4-yl)-1,3-dihydroisoindol-2-yl]biphenyl-4-carboxylic acid | C |
| 93 | 3-[1,3-Dioxo-5-(1H-[1,2,3]triazol-4-yl)-1,3-dihydroisoindol-2-yl]biphenyl-4-carboxylic acid | D |
| | amide | |
| 94 | 4-[1,3-Dioxo-5-(1H-tetrazol-5-yl)-1,3-dihydroisoindol-2-yl]biphenyl-3-carboxylic acid | A |
| 95 | 3-[1,3-Dioxo-5-(1H-tetrazol-5-yl)-1,3-dihydroisoindol-2-yl]biphenyl-4-carboxylic acid | A |
| 96 | 3-[1,3-Dioxo-5-(1H-tetrazol-5-yl)-1,3-dihydroisoindol-2-yl]biphenyl-4-carboxylic acid | D |
| | isopropyl ester | |
| 97 | 3-[1,3-Dioxo-5-(1H-tetrazol-5-yl)-1,3-dihydroisoindol-2-yl]biphenyl-4-carboxylic acid 2- | A |
| | dimethylamino-ethyl ester | |
| 98 | 3-[1,3-Dioxo-5-(1H-tetrazol-5-yl)-1,3-dihydroisoindol-2-yl]biphenyl-4-carboxylic acid amide | C |
| 99 | 3′,4′-Difluoro-3-[1-oxo-6-(1H-tetrazol-5-yl)-1,3-dihydroisoindol-2-yl]biphenyl-4-carboxylic | D |
| | acid methyl ester | |
| 100 | 3′,4′-Difluoro-3-[1-oxo-6-(1H-tetrazol-5-yl)-1,3-dihydroisoindol-2-yl]biphenyl-4-carboxylic | B |
| | acid | |
| 101 | 3′,4′-Difluoro-3-[1-oxo-6-(1H-tetrazol-5-yl)-1,3-dihydroisoindol-2-yl]biphenyl-4-carboxylic | D |
| | acid amide | |
| 102 | 2-(4-Methoxycarbonylbiphenyl-3-yl)-3-oxo-2,3-dihydro-1H-isoindole-5-carboxylic acid | E |
| 103 | 3-(6-Methanesulfonylaminocarbonyl-1-oxo-1,3-dihydroisoindol-2-yl)biphenyl-4-carboxylic | E |
| | acid methyl ester | |
| 104 | 3-(6-Methanesulfonylaminocarbonyl-1-oxo-1,3-dihydroisoindol-2-yl)biphenyl-4-carboxylic | A |
| | acid | |
| 105 | 3′,4′-Difluoro-3-(6-methanesulfonylaminocarbonyl-1-oxo-1,3-dihydroisoindol-2-yl)biphenyl- | E |
| | 4-carboxylic acid methyl ester | |
| 106 | 3′,4′-Difluoro-3-(6-methanesulfonylaminocarbonyl-1-oxo-1,3-dihydroisoindol-2-yl)biphenyl- | C |
| | 4-carboxylic acid | |
| 107 | 3′,4′-Difluoro-3-(6-methanesulfonylaminocarbonyl-1-oxo-1,3-dihydroisoindol-2-yl)biphenyl- | E |
| | 4-carboxylic acid amide | |
| 108 | 3′,4′-Difluoro-3-(6-methanesulfonylaminocarbonyl-1-oxo-1,3-dihydroisoindol-2-yl)biphenyl- | E |
| | 4-carboxylic acid 2-dimethylamino-ethyl ester | |
| 109 | 3′,4′-Difluoro-3-(6-methanesulfonylaminocarbonyl-1-oxo-1,3-dihydroisoindol-2-yl)biphenyl- | E |
| | 4-carboxylic acid 2-pyrrolidin-1-yl-ethyl ester | |
| 110 | 3′,4′-Difluoro-3-[1-oxo-6-(1H-[1,2,3]triazol-4-yl)-1,3-dihydroisoindol-2-yl]biphenyl-4- | E |
| | carboxylic acid methyl ester | |
| 111 | 3′,4′-Difluoro-3-[1-oxo-6-(1H-[1,2,3]triazol-4-yl)-1,3-dihydroisoindol-2-yl]biphenyl-4- | A |
| | carboxylic acid | |
| 112 | 3′,4′-Difluoro-3-[1-oxo-6-(1H-[1,2,3]triazol-4-yl)-1,3-dihydroisoindol-2-yl]biphenyl-4- | E |
| | carboxylic acid 2-dimethylamino-ethyl ester | |
| 112a | 3′,4′-Difluoro-3-[1-oxo-6-(1H-[1,2,3]triazol-4-yl)-1,3-dihydroisoindol-2-yl]biphenyl-4- | ND |
| | carboxylic acid (2-oxo-1,3-oxazolidin-5-yl)methyl ester | |
| 112b | 3′,4′-Difluoro-3-[1-oxo-6-(1H-[1,2,3]triazol-4-yl)-1,3-dihydroisoindol-2-yl]biphenyl-4- | ND |
| | carboxylic acid 2,3-dihydroxypropyl ester | |
| 112c | 3′,4′-Difluoro-3-[1-oxo-6-(1H-[1,2,3]triazol-4-yl)-1,3-dihydroisoindol-2-yl]biphenyl-4- | ND |
| | carboxylic acid butan-2-yl ester | |
| 112d | 3′,4′-Difluoro-3-[1-oxo-6-(1H-[1,2,3]triazol-4-yl)-1,3-dihydroisoindol-2-yl]biphenyl-4- | ND |
| | carboxylic acid 1-(dimethylamino)propan-2-yl ester | |
| 113 | 3′,4′-Difluoro-3-[1-oxo-6-(1H-[1,2,3]triazol-4-yl)-1,3-dihydroisoindol-2-yl]biphenyl-4- | E |
| | carboxylic acid amide | |
| 114 | 3′,4′-Difluoro-3-[1-oxo-6-(1H-[1,2,3]triazol-4-yl)-1,3-dihydroisoindol-2-yl]biphenyl-4- | ND |
| | carboxylic acid 2-methoxy-ethyl ester | |
| 115 | 3′,4′-Difluoro-3-[1-oxo-6-(1H-[1,2,3]triazol-4-yl)-1,3-dihydroisoindol-2-yl]biphenyl-4- | E |
| | carboxylic acid 2-pyrrolidin-1-yl-ethyl ester | |
| 116 | 3′,4′-Difluoro-3-[1-oxo-6-(1H-[1,2,3]triazol-4-yl)-1,3-dihydro-isoindol-2-yl]-biphenyl-4- | D |
| | carboxylic acid (5-methyl-2-oxo-[1,3]dioxol-4-yl)methyl ester | |
| 117 | 3′,4′-Difluoro-3-[1-oxo-6-(1H-[1,2,3]triazol-4-yl)-1,3-dihydro-isoindol-2-yl]-biphenyl-4- | E |
| | carboxylic acid tert-butyl ester | |
| 118 | 3′,4′-Difluoro-3-[1-oxo-6-(1H-[1,2,3]triazol-4-yl)-1,3-dihydro-isoindol-2-yl]-biphenyl-4- | E |
| | carboxylic acid isopropyl ester | |
| 119 | 2-(4-Carbamoyl-3′,4′-difluorobiphenyl-3-yl)-3-oxo-2,3-dihydro-1H-isoindole-5-carboxylic | D |
| | acid | |
| 120 | 2-[4-(2-Dimethylamino-ethoxycarbonyl)-3′,4′-difluorobiphenyl-3-yl]-3-oxo-2,3-dihydro-1H- | D |
| | isoindole-5-carboxylic acid | |
| 121 | 3′,4′-Difluoro-3-(1-oxo-6-tetrazol-1-yl-1,3-dihydroisoindol-2-yl)biphenyl-4-carboxylic acid | E |
| | methyl ester | |
| 122 | 3′,4′-Difluoro-3-(1-oxo-6-tetrazol-1-yl-1,3-dihydroisoindol-2-yl)biphenyl-4-carboxylic acid | B |
| 123 | 2-(4-Carbamoyl-3′,4′-difluorobiphenyl-3-yl)-3-oxo-2,3-dihydro-1H-isoindole-5-carboxylic | E |
| | acid butyl ester | |
| 124 | 3′,4′-Difluoro-3-(6-methanesulfonylaminocarbonyl-1-oxo-1,3-dihydro-isoindol-2-yl)- | E |
| | biphenyl-4-carboxylic acid 3-dimethylamino-propyl ester | |
| 125 | 3′,4′-Difluoro-3-(6-methanesulfonylaminocarbonyl-1-oxo-1,3-dihydro-isoindol-2-yl)- | E |
| | biphenyl-4-carboxylic acid isopropyl ester | |
| 126 | 2-(4-Carboxy-3′,4′-difluoro-biphenyl-3-yl)-3-oxo-2,3-dihydro-1H-isoindole-5-carboxylic acid | E |
| 127 | 2-(3′,4′-Difluoro-4-methoxycarbonylbiphenyl-3-yl)-3-oxo-2,3-dihydro-1H-isoindole-5- | E |
| | carboxylic acid methyl ester | |
| 128 | 2-(4-Carboxy-3′,4′-difluoro-biphenyl-3-yl)-3-oxo-2,3-dihydro-1H-isoindole-5-carboxylic acid | D |
| | butyl ester | |
| 129 | 2-(4-Carboxy-3′,4′-difluorobiphenyl-3-yl)-3-oxo-2,3-dihydro-1H-isoindole-5-carboxylic acid | E |
| | 2-dimethylamino-ethyl ester | |
| 130 | 3-(6-Acetylamino-1-oxo-1,3-dihydroisoindol-2-yl)-3′,4′-difluoro-biphenyl-4-carboxylic acid | E |
| 131 | 3′,4′-Difluoro-3-(6-methanesulfonylamino-1-oxo-1,3-dihydro-isoindol-2-yl)-biphenyl-4- | E |
| | carboxylic acid | |
| 132 | 3′,4′-Difluoro-3-[1-oxo-6-(toluene-4-sulfonylamino)-1,3-dihydro-isoindol-2-yl]-biphenyl-4- | D |
| | carboxylic acid | |
| 133 | 2-(3-Cyano-4,5-diphenylthiophen-2-yl)-1,3-dioxo-2,3-dihydro-1H-isoindole-5-carboxylic acid | A |
| 134 | 2-(3-Carboxybiphenyl-4-yl)-1,3-dioxo-2,3-dihydro-1H-isoindole-5-carboxylic acid | A (A) |
| 135 | N-(Benzenesulfonyl)-2-(3-(1H-tetrazol-5-yl)-[1,1′-biphenyl]-4-yl)-1,3-dioxo-2,3-dihydro-1H- | D |
| | isoindole-5-carboxamide | |
| 136 | (2S)-2-({2-[3-Cyano-4-(4-methoxyphenyl)-5-methyl-thiophen-2-yl]-1,3-dioxo-2,3-dihydro- | C |
| | 1H-isoindole-5-carbonyl}-amino)-3-(4-hydroxyphenyl)propanoic acid | |
| 137 | (2S)-2-({2-[3-Cyano-4-(4-methoxyphenyl)-5-methyl-thiophen-2-yl]-1,3-dioxo-2,3-dihydro- | B |
| | 1H-isoindole-5-carbonyl}-amino)-3-phenylpropanoic acid | |
| 138 | 2-(4-Carboxy-3′,4′-difluoro[1,1′-biphenyl]-3-yl)-1,3-dioxo-2,3-dihydro-1H-isoindole-5- | A (A) |
| | carboxylic acid | |
| 139 | 2-(4-Carboxy-2′,3′,4′-trifluoro[1,1′-biphenyl]-3-yl)-1,3-dioxo-2,3-dihydro-1H-isoindole-5- | A |
| | carboxylic acid | |
| 140 | 2-(4-Carboxy-2′,4′,5′-trifluoro[ 1,1′-biphenyl]-3-yl)-1,3-dioxo-2,3-dihydro-1H-isoindole-5- | A |
| | carboxylic acid | |
| 141 | 2-(4-Carboxy-4′-methyl[1,1′-biphenyl]-3-yl)-1,3-dioxo-2,3-dihydro-1H-isoindole-5-carboxylic | A |
| | acid | |
| 142 | 2-(4-Carboxy-2′,4′-dichloro[1,1′-biphenyl]-3-yl)-1,3-dioxo-2,3-dihydro-1H-isoindole-5- | A |
| | carboxylic acid | |
| 143 | 2-(4-Carboxy-4′-chloro-3′-fluoro[1,1′-biphenyl]-3-yl)-1,3-dioxo-2,3-dihydro-1H-isoindole-5- | A (A) |
| | carboxylic acid | |
| 144 | 2-(4-Carboxy-3′-fluoro-4′-methoxy[1,1′-biphenyl]-3-yl)-1,3-dioxo-2,3-dihydro-1H-isoindole- | A |
| | 5-carboxylic acid | |
| 144a | 2-(4-carboxy-3′,4′-difluoro[1,1′-biphenyl]-3-yl)-6-hydroxy-1,3-dioxo-2,3-dihydro-1H- | A (A) |
| | isoindole-5-carboxylic acid | |
| 144b | 3-[5-chloro-1,3-dioxo-6-(1H-1,2,3-triazol-5-yl)-2,3-dihydro-1H-isoindol-2-yl]-3′,4′- | A (A) |
| | difluoro[1,1′-biphenyl]-4-carboxylic acid | |
| 144c | 3-[5-chloro-1,3-dioxo-6-(1H-1,2,3-triazol-5-yl)-2,3-dihydro-1H-isoindol-2-yl][1,1′-biphenyl]- | A (A) |
| | 4-carboxylic acid | |
| 145 | 2-(3-cyanobiphenyl-4-yl)-1,3-dioxoisoindoline-5-carboxylic acid | E |
| 146 | 2-(4-(4-fluorophenyl)-5-phenylpyridin-2-yl)-5-(1H-1,2,3-triazol-4-yl)isoindoline-1,3-dione | E |
| 147 | 2-(1-methyl-2-oxo-5-phenyl-1,2-dihydropyridin-3-yl)-5-(1H-1,2,3-triazol-4-yl)isoindoline- | E |
| | 1,3-dione | |
| 148 | 3-(1,3-dioxo-5-(1H-1,2,3-triazol-4-yl)isoindolin-2-yl)biphenyl-4-sulfonamide | E |
| 149 | 2-(2-(3-cyano-4-(4-methoxyphenyl)-5-methylthiophen-2-yl)-1,3-dioxoisoindoline-5- | E |
| | carboxamido)acetic acid | |
| 150 | methyl 2-(4-hydroxybiphenyl-3-yl)-1,3-dioxoisoindoline-5-carboxylate | ND |
| 151 | 2-(4-methylpiperazin-1-yl)ethyl 2-(3-cyano-4-(4-methoxyphenyl)-5-methylthiophen-2-yl)-1,3- | ND |
| | dioxoisoindoline-5-carboxylate | |
| 152 | methyl 4-(1,3-dioxo-5-(phenylsulfonylcarbamoyl)isoindolin-2-yl)biphenyl-3-carboxylate | E |
| 153 | 2-aminoethyl 3-(1,3-dioxo-5-(1H-1,2,3-triazol-4-yl)isoindolin-2-yl)biphenyl-4-carboxylate | E |
| 154 | 2-(2-(1H-tetrazol-5-yl)-5-methoxyphenyl)-5-(1H-1,2,3-triazol-4-yl)isoindoline-1,3-dione | D |
| 155 | 2-(4-methylpiperazin-1-yl)ethyl 3′,4′-difluoro-3-(6-(hydroxymethyl)-1-oxoisoindolin-2- | E |
| | yl)biphenyl-4-carboxylate | |
| 156 | isopropyl 3′,4′-difluoro-3-(6-(hydroxymethyl)-1-oxoisoindolin-2-yl)biphenyl-4-carboxylate | E |
| 157 | methyl 3′,4′-difluoro-3-(6-(hydroxymethyl)-1-oxoisoindolin-2-yl)biphenyl-4-carboxylate | E |
| 158 | 3′,4′-difluoro-3-(6-(hydroxymethyl)-1-oxoisoindolin-2-yl)biphenyl-4-carboxylic acid | D |
| 159 | ethyl 3′,4′-difluoro-3-(6-(hydroxymethyl)-1-oxoisoindolin-2-yl)biphenyl-4-carboxylate | E |
| 160 | 3-(6-carbamoyl-1-oxoisoindolin-2-yl)-3′,4′-difluorobiphenyl-4-carboxylic acid | D |
| 161 | 3′,4′-difluoro-3-(6-(5-(hydroxymethyl)-1H-1,2,3-triazol-4-yl)-1-oxoisoindolin-2-yl)biphenyl- | D |
| | 4-carboxylic acid | |
| 162 | 3-(6-(5-benzoyl-1H-1,2,3-triazol-4-yl)-1-oxoisoindolin-2-yl)-3′,4′-difluorobiphenyl-4- | B |
| | carboxylic acid | |
| 163 | 3′,4′-difluoro-3-(1-oxo-6-(5-phenyl-1H-1,2,3-triazol-4-yl)isoindolin-2-yl)biphenyl-4- | B |
| | carboxylic acid | |
| 164 | 3-(6-(5-carbamoyl-1H-1,2,3-triazol-4-yl)-1-oxoisoindolin-2-yl)-3′,4′-difluorobiphenyl-4- | C |
| | carboxylic acid | |
| 165 | 3-(6-(5-(dimethylcarbamoyl)-1H-1,2,3-triazol-4-yl)-1-oxoisoindolin-2-yl)-3′,4′- | C |
| | difluorobiphenyl-4-carboxylic acid | |
| 166 | ethyl 3′,4′-difluoro-3-[1-oxo-6-(1H-1,2,3-triazol-5-yl)-2,3-dihydro-1H-isoindol-2-yl][1,1′- | E (E) |
| | biphenyl]-4-carboxylate | |
| 167 | ethyl 3-[1,3-dioxo-5-(1H-1,2,3-triazol-5-yl)-1,3-dihydro-2H-isoindol-2-yl]-3′,4′-difluoro[1,1′- | E |
| | biphenyl]-4-carboxylate | |
| 168 | 2-(acetyloxy)ethyl 3-[1,3-dioxo-5-(1H-1,2,3-triazol-5-yl)-2,3-dihydro-1H-isoindol-2-yl]-3′,4′- | E |
| | difluoro[1,1′-biphenyl]-4-carboxylate | |
| 169 | 6-bromo-2-(4-carboxy-3′,4′-difluoro[1,1′-biphenyl]-3-yl)-1,3-dioxo-2,3-dihydro-1H-isoindole- | A |
| | 5-carboxylic acid | |
| 170 | 2-(4-carboxy-3′,4′-difluoro[1,1′-biphenyl]-3-yl)-6-methoxy-1,3-dioxo-2,3-dihydro-1H- | A |
| | isoindole-5-carboxylic acid | |

Examples of enzymatic activity of some compounds of Formula (VII)

For the purposes of enzymatic activity of some compounds of Formula (VII)

a numbering of examples restarted from 1. This below table contains such restarted numbering. Example #1 is a last one in below table, and example #86 is a first one.

| | | Enzymatic |
|---|---|---|
| | | PFKFB3 |
| Patent | | activity: |
| Example | Name | IC50 (μM) |
| Example 86 | 2-{[4-carboxy-2′-(1H-imidazol-4-yl)-[1,1′-biphenyl]-3-yl]carbamoyl}-5-hydroxybenzene- | A |
| | 1,4-dicarboxylic acid | |
| Example 85 | 4-{[4-carboxy-2′-(1H-imidazol-4-yl)-[1,1′-biphenyl]-3-yl]carbamoyl}-6-hydroxybenzene- | A |
| | 1,3-dicarboxylic acid | |
| Example 84 | 2-{[5-(2,4-dichlorophenyl)-2-oxo-1,2-dihydropyridin-3-yl]carbamoyl}-4-{[dimethyl(oxo)- | E |
| | λ⁶-sulfanylidene]carbamoyl}benzoic acid | |
| Example 83 | 3-[2-carboxy-5-(1,2-dihydroxyethyl)benzamido]-3′,4′-difluoro-[1,1′-biphenyl]-4- | E |
| | carboxylic acid | |
| Example 82 | 3-[2-carboxy-4-(1,2-dihydroxyethyl)benzamido]-3′,4′-difluoro-[1,1′-biphenyl]-4- | D |
| | carboxylic acid | |
| Example 81 | 2-({4-carboxy-2′,4′-dichloro-[1,1′-biphenyl]-3-yl}carbamoyl)-5-fluorobenzene-1,4- | A |
| | dicarboxylic acid | |
| Example 80 | 4-({2′,4′-dichloro-4-[4-(dimethylamino)butanoyl]-[1,1′-biphenyl]-3-yl}carbamoyl)-6- | A |
| | hydroxybenzene-1,3-dicarboxylic acid | |
| Example 79 | 2-[(2′,4′-dichloro-4-{[(dimethylcarbamoyl)methoxy]carbonyl}-[1,1′-biphenyl]-3- | A |
| | yl)carbamoyl]-5-hydroxybenzene-1,4-dicarboxylic acid | |
| Example 78 | 2-({2′,4′-dichloro-4-[4-(dimethylamino)butanoyl]-[1,1′-biphenyl]-3-yl}carbamoyl)-5- | C |
| | hydroxybenzene-1,4-dicarboxylic acid | |
| Example 77 | 2-[(2′,4′-dichloro-4-{[(1-methylazetidin-3-yl)oxy]carbonyl}-[1,1′-biphenyl]-3- | A |
| | yl)carbamoyl]-5-hydroxybenzene-1,4-dicarboxylic acid | |
| Example 76 | 2-[(2′,4′-dichloro-4-{[(1-methylazetidin-2-yl)methoxy]carbonyl}-[1,1′-biphenyl]-3- | A |
| | yl)carbamoyl]-5-hydroxybenzene-1,4-dicarboxylic acid | |
| Example 75 | 2-[(2′,4′-dichloro-4-{[(1-methylpyrrolidin-3-yl)oxy]carbonyl}-[1,1′-biphenyl]-3- | B |
| | yl)carbamoyl]-5-hydroxybenzene-1,4-dicarboxylic acid | |
| Example 74 | 2-[(2′,4′-dichloro-4-{[2-(dimethylamino)propoxy]carbonyl}-[1,1′-biphenyl]-3- | A |
| | yl)carbamoyl]-5-hydroxybenzene-1,4-dicarboxylic acid | |
| Example 73 | 2-{[2′,4′-dichloro-4-({[1-(dimethylamino)propan-2-yl]oxy}carbonyl)-[1,1′-biphenyl]-3- | A |
| | yl]carbamoyl}-5-hydroxybenzene-1,4-dicarboxylic acid | |
| Example 72 | 5-{[dimethyl(oxo)-λ⁶-sulfanylidene]carbamoyl}-2-{[4-(4,4-dimethylpiperidin-1- | E |
| | yl)pyridin-2-yl]carbamoyl}benzoic acid | |
| Example 71 | 4-({4-[(2-aminoethoxy)carbonyl]-2′,4′-dichloro-[1,1′-biphenyl]-3-yl}carbamoyl)-6- | A |
| | hydroxybenzene-1,3-dicarboxylic acid | |
| Example 70 | 4-[(2′,4′-dichloro-4-{[(dimethylcarbamoyl)methoxy]carbonyl}-[1,1′-biphenyl]-3- | A |
| | yl)carbamoyl]-6-hydroxybenzene-1,3-dicarboxylic acid | |
| Example 69 | 4-[(2′,4′-dichloro-4-{[(1-methylazetidin-3-yl)oxy]carbonyl}-[1,1′-biphenyl]-3- | A |
| | yl)carbamoyl]-6-hydroxybenzene-1,3-dicarboxylic acid | |
| Example 68 | 4-[(2′,4′-dichloro-4-{[(1-methylazetidin-2-yl)methoxy]carbonyl}-[1,1′-biphenyl]-3- | A |
| | yl)carbamoyl]-6-hydroxybenzene-1,3-dicarboxylic acid | |
| Example 67 | 4-[(2′,4′-dichloro-4-{[(1-methylpyrrolidin-3-yl)oxy]carbonyl}-[1,1′-biphenyl]-3- | A |
| | yl)carbamoyl]-6-hydroxybenzene-1,3-dicarboxylic acid | |
| Example 66 | 4-[(2′,4′-dichloro-4-{[2-(dimethylamino)propoxy]carbonyl}-[1,1′-biphenyl]-3- | A |
| | yl)carbamoyl]-6-hydroxybenzene-1,3-dicarboxylic acid | |
| Example 65 | 4-{[2′,4′-dichloro-4-({[1-(dimethylamino)propan-2-yl]oxy}carbonyl)-[1,1′-biphenyl]-3- | A |
| | yl]carbamoyl}-6-hydroxybenzene-1,3-dicarboxylic acid | |
| Example 64 | 2-{[2-carboxy-5-(4,4-dimethylcyclohexyl)phenyl]carbamoyl}-5-fluorobenzene-1,4- | A |
| | dicarboxylic acid | |
| Example 63 | 4-{[2-carboxy-5-(4,4-dimethylcyclohexyl)phenyl]carbamoyl}-6-fluorobenzene-1,3-dicarboxylic acid | A |
| Example 62 | 4-[(5-{2-azabicyclo[2.2.1]heptan-2-yl}-2-carboxyphenyl)carbamoyl]-6-hydroxybenzene- | A |
| | 1,3-dicarboxylic acid | |
| Example 61 | 2-[(5-{2-azabicyclo[2.2.1]heptan-2-yl}-2-carboxyphenyl)carbamoyl]-5-hydroxybenzene- | A |
| | 1,4-dicarboxylic acid | |
| Example 60 | 2-{[2′,4′-dichloro-4-(ethoxycarbonyl)-[1,1′-biphenyl]-3-yl]carbamoyl}-5-hydroxybenzene- | A |
| | 1,4-dicarboxylic acid | |
| Example 59 | 4-({4-carboxy-2′,4′-dichloro-[1,1′-biphenyl]-3-yl}carbamoyl)-6-fluorobenzene-1,3- | A |
| | dicarboxylic acid | |
| Example 58 | 4-[(5-{2-azabicyclo[2.2.1]heptan-2-yl}-2-carboxyphenyl)carbamoyl]-6-hydroxybenzene- | A |
| | 1,3-dicarboxylic acid | |
| Example 57 | 2-{[2-carboxy-5-(4,4-dimethylcyclohexyl)phenyl]carbamoyl}-5-hydroxybenzene-1,4- | A |
| | dicarboxylic acid | |
| Example 56 | 2-{[2-carboxy-5-(4,4-dimethylpiperidin-1-yl)phenyl]carbamoyl}-5-hydroxybenzene-1,4- | A |
| | dicarboxylic acid | |
| Example 55 | 4-{[2′,4′-dichloro-4-(ethoxycarbonyl)-[1,1′-biphenyl]-3-yl]carbamoyl}-6-hydroxybenzene- | A |
| | 1,3-dicarboxylic acid | |
| Example 54 | 2-[(2′,4′-dichloro-4-{[3-(dimethylamino)propoxy]carbonyl}-[1,1′-biphenyl]-3- | A |
| | yl)carbamoyl]-5-hydroxybenzene-1,4-dicarboxylic acid | |
| Example 53 | 4-{[2-carboxy-5-(4,4-dimethylpiperidin-1-yl)phenyl]carbamoyl}-6-hydroxybenzene-1,3- | A |
| | dicarboxylic acid | |
| Example 52 | 4-{[2-carboxy-5-(4,4-dimethylcyclohexyl)phenyl]carbamoyl}-6-hydroxybenzene-1,3- | A |
| | dicarboxylic acid | |
| Example 51 | 4-[(2′,4′-dichloro-4-{[3-(dimethylamino)propoxy]carbonyl}-[1,1′-biphenyl]-3- | A |
| | yl)carbamoyl]-6-hydroxybenzene-1,3-dicarboxylic acid | |
| Example 50 | 2-[(2′,4′-dichloro-4-{[2-(dimethylamino)ethoxy]carbonyl}-[1,1′-biphenyl]-3- | B |
| | yl)carbamoyl]-4-{[dimethyl(oxo)-λ⁶-sulfanylidene]carbamoyl}benzoic acid | |
| Example 49 | 2-[(2′,4′-dichloro-4-{[2-(dimethylamino)ethoxy]carbonyl}-[1,1′-biphenyl]-3- | A |
| | yl)carbamoyl]-5-{[dimethyl(oxo)-λ⁶-sulfanylidene]carbamoyl}benzoic acid | |
| Example 46 | 2-({4-carboxy-3′,4′-difluoro-[1,1′-biphenyl]-3-yl}carbamoyl)-5-chlorobenzene-1,4- | A |
| | dicarboxylic acid | |
| Example 45 | 4-({4-carboxy-3′,4′-difluoro-[1,1′-biphenyl]-3-yl}carbamoyl)-6-chlorobenzene-1,3- | A |
| | dicarboxylic acid | |
| Example 44 | 2-({4-carboxy-2′,4′-dichloro-[1,1′-biphenyl]-3-yl}carbamoyl)-5-hydroxybenzene-1,4- | A |
| | dicarboxylic acid | |
| Example 43 | 4-({4-carboxy-2′,4′-dichloro-[1,1′-biphenyl]-3-yl}carbamoyl)-6-hydroxybenzene-1,3- | A |
| | dicarboxylic acid | |
| Example 42 | 2′,4′-dichloro-3-(5-{[dimethyl(oxo)-λ⁶-sulfanylidene]carbamoyl}-2-(dimethylcarbamoyl)- | C |
| | 4-hydroxybenzamido)-[1,1′-biphenyl]-4-carboxylic acid | |
| Example 41 | 3-(2-carboxy-4-{[dimethyl(oxo)-λ⁶-sulfanylidene]carbamoyl}benzamido)-3′,4′-difluoro- | A |
| | [1,1′-biphenyl]-4-carboxylic acid | |
| Example 40 | 2-[(4-phenyl-1,3-thiazol-2-yl)carbamoyl]benzene-1,4-dicarboxylic acid | E |
| Example 39 | 2-[(5-nitro-1,3-thiazol-2-yl)carbamoyl]benzene-1,4-dicarboxylic acid | B |
| Example 38 | 4-[(5-nitro-1,3-thiazol-2-yl)carbamoyl]benzene-1,3-dicarboxylic acid | A |
| Example 37 | 2-({3′,4′-difluoro-4-hydroxy-[1,1′-biphenyl]-3-yl}carbamoyl)-5-hydroxybenzene-1,4- | A |
| | dicarboxylic acid | |
| Example 36 | 3-(2-carboxy-5-{[dimethyl(oxo)-λ⁶-sulfanylidene]carbamoyl}benzamido)-3′,4′-difluoro- | B |
| | [1,1′-biphenyl]-4-carboxylic acid | |
| Example 35 | 3-(2-carboxy-4-{[imino(methyl)methylidene-λ⁶-sulfanyl]carbamoyl}benzamido)-2′,4′- | A |
| | dichloro-[1,1′-biphenyl]-4-carboxylic acid | |
| Example 34 | 3-(2-carboxy-5-{[imino(methyl)methylidene-λ⁶-sulfanyl]carbamoyl}benzamido)-2′,4′- | B |
| | dichloro-[1,1′-biphenyl]-4-carboxylic acid | |
| Example 33 | 4-((3-cyano-4-(4-methoxyphenyl)-5-methylthiophen-2-yl)carbamoyl)-6- | A |
| | hydroxyisophthalic acid | |
| Example 32 | 3-[4-carboxy-2-(dimethylcarbamoyl)-5-hydroxybenzamido]-2′,4′-dichloro-[1,1′-biphenyl]- | B |
| | 4-carboxylic acid | |
| Example 30 | 4-{[3-cyano-4-(4-methoxyphenyl)-5-methylthiophen-2-yl]carbamoyl}benzene-1,3- | A |
| | dicarboxylic acid | |
| Example 29 | 2-{[3-cyano-4-(4-methoxyphenyl)-5-methylthiophen-2-yl]carbamoyl}benzene-1,4- | D |
| | dicarboxylic acid | |
| Example 28 | 2-({4-carboxy-3′,4′-difluoro-[1,1′-biphenyl]-3-yl}carbamoyl)-5-hydroxybenzene-1,4- | A |
| | dicarboxylic acid | |
| Example 27 | 4-({3′,4′-difluoro-4-hydroxy-[1,1′-biphenyl]-3-yl}carbamoyl)-6-hydroxybenzene-1,3- | A |
| | dicarboxylic acid | |
| Example 26 | 3-[5-chloro-2-(dimethylsulfamoyl)-4-(2H-1,2,3-triazol-4-yl)benzamido]-3′,4′-difluoro- | C |
| | [1,1′-biphenyl]-4-carboxylic acid | |
| Example 23 | 4-({4-carboxy-4′-fluoro-[1,1′-biphenyl]-3-yl}carbamoyl)benzene-1,3-dicarboxylic acid | A |
| Example 22 | 3-[2-carboxy-4-(2H-1,2,3-triazol-4-yl)benzamido]-3′,4′-difluoro-[1,1′-biphenyl]-4- | A |
| | carboxylic acid | |
| Example 21 | 3-[2-carboxy-5-(2H-1,2,3-triazol-4-yl)benzamido]-3′,4′-difluoro-[1,1′-biphenyl]-4- | A |
| | carboxylic acid | |
| Example 20 | 3-{[2-carboxy-4-chloro-5-(2H-1,2,3-triazol-4-yl)phenyl]carbamoyl}-3′,4′-difluoro-[1,1′- | C |
| | biphenyl]-4-carboxylic acid | |
| Example 19 | 3-{[2-carboxy-5-chloro-4-(2H-1,2,3-triazol-4-yl)phenyl]carbamoyl}-3′,4′-difluoro-[1,1′- | A |
| | biphenyl]-4-carboxylic acid | |
| Example 18 | 2-{[1,1′-biphenyl]-3-amido}-4-chloro-5-(2H-1,2,3-triazol-4-yl)benzoic acid | D |
| Example 17 | 4-{[1,1′-biphenyl]-3-amido}benzene-1,3-dicarboxylic acid | B |
| Example 16 | 4-({4-carboxy-3′,4′-difluoro-[1,1′-biphenyl]-3-yl}carbamoyl)-6-hydroxybenzene-1,3- | E |
| | dicarboxylic acid | |
| Example 15 | N1-[3-cyano-4-(4-methoxyphenyl)-5-methylthiophen-2-yl]-4-(hydroxymethyl)-N2- | E |
| | methylbenzene-1,2-dicarboxamide | |
| Example 14 | 4-{[3-cyano-4-(4-methoxyphenyl)-5-methylthiophen-2-yl]carbamoyl}-3-{[2- | E |
| | (dimethylamino)ethyl]carbamoyl}benzoic acid | |
| Example 12 | 4-{[3-cyano-4-(4-methoxyphenyl)-5-methylthiophen-2-yl]carbamoyl}-3- | E |
| | (hydroxymethyl)benzoic acid | |
| Example 11 | 3-carbamoyl-4-[(3-cyano-4,5-diphenylthiophen-2-yl)carbamoyl]benzoic acid | A |
| Example 10 | 5-[(1-carboxy-2-hydroxyethyl)carbamoyl]-2-{[3-cyano-4-(4-methoxyphenyl)-5- | E |
| | methylthiophen-2-yl]carbamoyl}benzoic acid | |
| Example 9 | methyl 2-{[3-cyano-4-(4-methoxyphenyl)-5-methylthiophen-2-yl]carbamoyl}-5-(2H- | D |
| | 1,2,3-triazol-4-yl)benzoate | |
| Example 8 | N1-[3-cyano-4-(4-methoxyphenyl)-5-methylthiophen-2-yl]-4-(2H-1,2,3-triazol-4- | D |
| | yl)benzene-1,2-dicarboxamide | |
| Example 7 | 3-({[2-carboxy-5-(2H-1,2,3-triazol-4-yl)phenyl]methyl}amino)-3′,4′-difluoro-[1,1′- | D |
| | biphenyl]-4-carboxylic acid | |
| Example 6 | 3-({[2-carboxy-4-(2H-1,2,3-triazol-4-yl)phenyl]methyl}amino)-3′,4′-difluoro-[1,1′-biphenyl]- | A |
| | 4-carboxylic acid | |
| Example 5 | 4-({4-carboxy-[1,1′-biphenyl]-3-yl}carbamoyl)benzene-1,3-dicarboxylic acid | B |
| Example 4 | 3-(2-amino-4-carboxybenzamido)-[1,1′-biphenyl]-4-carboxylic acid | E |
| Example 1 | 2-({4-carboxy-4′-fluoro-[1,1′-biphenyl]-3-yl}carbamoyl)benzene-1,4-dicarboxylic acid | B |

### Example II: Neuroprotection

The excitotoxicity model in mouse primary neuronal culture was used to assess the compounds disclosed herein as neuroprotective agents. Compounds 3′,4′-Difluoro-3-[1-oxo-6-(1H-[1,2,3]triazol-4-yl)-1,3-dihydroisoindol-2-yl]biphenyl-4-carboxylic acid (compound 111), 3′,4′-Difluoro-3-[1-oxo-6-(1H-[1,2,3]triazol-4-yl)-1,3-dihydro-isoindol-2-yl]-biphenyl-4-carboxylic acid isopropyl ester (compound 118) or ethyl 3′,4′-difluoro-3-[1-oxo-6-(1H-1,2,3-triazol-5-yl)-1,3-dihydro-2H-isoindol-2-yl][1,1′-biphenyl]-4-carboxylate (compound 166) were added to the cell culture and then exposed to glutamate (100 μM for 15 minutes), apoptotic death was assessed by measuring active caspase-3 in neuronal extracts using an immunofluorescence method based on an active caspase-3-FITC staining kit. Compounds 118 and 166 prevented the apoptotic death of neurons in this excitotoxic model at 0.1 μM, 1 μM and 10 μM, compound 111 demonstrated protective effect at 0.1 μM and 1 μM. Additional details for neuronal cell culture preparation can be found in Proc Natl Acad Sci USA, Complex I assembly into supercomplexes determines differential mitochondrial ROS production in neurons and astrocytes, Lopez-Fabuel I. et al., details of excitotoxicity model can be found in Nature Cell Biology 11, 747-752 (2009), The bioenergetic and antioxidant status of neurons is controlled by continuous degradation of a key glycolytic enzyme by APC/C-Cdh1, Herrero-Mendez A. et al.

### Example III: Immunosuppression (Prophetic)

In this and in the following examples compound 1 and GO-672 is used interchangeably with 4-({4-carboxy-2′,4′-dichloro-[1,1′-biphenyl]-3-yl}carbamoyl)-6-hydroxybenzene-1,3-dicarboxylic acid.

Human blood mononuclear cells can be used to assess the compounds disclosed herein as immunosuppressive agent. Cells are incubated in presence of any one of the compounds disclosed herein, e.g. but not limited to Compound 1 and AZ-67 each at 0, 0.1 μM, 1 μM, 10 μM and 100 μM for 4-24 hours. Immunosuppression is measured as decrease of cytokines level (for example one of these cytokines: L-1α, IL-1RA, IL-1β, IL-2, IL-3, IL-4, IL-5, IL-6, IL-8, IL-10, IL-12μ40, MCP-1, MIP1α, INFγ, TNFα, GM-CSF, IL-17, sCD40). The compounds of this invention, including but not limited to Compound 1 and AZ-67 demonstrate immunosuppressive activity in such and in other standard immunosuppression tests.

### Example IV: Activity Against Viral Infections on the Example of Influenza (Prophetic)

An in vitro model of influenza infection can be used to assess the compounds disclosed herein as antiviral agents. The cell culture of MDCK is plated in a medium with different levels of glucose and incubated for 24 hours at 37° C. Then cells are treated with any compound disclosed herein, e.g. but not limited to Compound 1 and AZ-67 eachb at 0, 0.1 μM, 1 μM, 10 μM and 100 μM and incubated for 0-24 hours at 37° C. Then cells are infected with influenza A H1N1 and 24 hours after are washed with PBS and fresh medium including compounds are added. Antiviral effect can be measured using an anti-HA primary antibody by immunostain method. The compounds of this invention, including but not limited to Compound 1 and AZ-67 demonstrate antiviral activity in such and in other standard antiviral tests.

### Example V: Acute Diseases and Conditions Treatment-30-Days Treatment. (Prophetic)

An animal model for any of the acute conditions or diseases mentioned in this application (a mice or other mammal disease model known to the expert in the area of specific disease research) is used. The animal is given intraperitoneally an effective amount of the solution of any of compounds disclosed herein, e.g. but not limited to Compound 1 and AZ-67 each (for example each −20 mg per kg) once a day for a period of at least 30 days. The amelioration of at least one of the major symptoms of the corresponding disease or conditions can be identified by the methods known in the art. The compounds of this invention, including but not limited to Compound 1 and AZ-67 demonstrate efficacy in such disease model.

### Example VI: Chronic Administration (Prophetic)

An animal model of the any of the chronic or lifelong diseases mentioned in this application (a mice or other mammal disease model known to the expert in the area of specific disease research) is used. The animal is given intraperitoneally an effective amount of solution of the any of the compounds disclosed herein, e.g. but not limited to Compound 1 and AZ-67 each (for example-20 mg per kg) once a day for a period of lifetime. The amelioration of at least one of the symptoms of the corresponding disease can be identified by the methods known in the art. In case of side effects occurrence which are more grave than the damage from the disease or condition under treatment the administration of the drug can be interrupted or dose decreased temporarily for the time until the side effects disappear or decrease to the acceptable level after what the administration or full dose can be resumed until the next occurrence of the side effects graver than the disease under treatment, in which case the treatment can be again interrupted temporarily as shown above. The compounds of this invention, including but not limited to Compound 1 and AZ-67 demonstrate efficacy in such disease model.

### Example VII: Prophylaxis. (Prophetic)

A mice or other mammal disease model known to the expert in the area of specific disease research is given intraperitoneally an effective amount of the any of the compounds disclosed herein, e.g. but not limited to Compound 1 and AZ-67 (for example −20 mg per kg each) once a day for a period of at least 7 days before the induction or expected start of the corresponding disease or condition and continue administration during the induction of the disease or the expected start, at the same time the control animal or group of animal should not administer the compound of invention. After that the attempt of disease induction to the treated and control animal or group of animals or the expected date of the diseases or condition start the absence or amelioration of at least one of the major symptoms of corresponding disease can be identified by the methods known in the art in comparison with the control animal or group of animal which did not administer the compound of invention. In other prophetic example the same can be done by the alike administration at least one day before the induction or expected start of the corresponding disease or condition and continue administration during the induction of the disease or the expected start. The compounds of this invention, including but not limited to Compound 1 and AZ-67 demonstrate efficacy in such disease model.

### Example VIII: Radiosensitization of the Cancer Cells (Prophetic)

Clonogenic survival could be studied with BJ TERT, BJ RAS, or U2OS cell line.

BJ TERT (498 cells), BJ RAS (201 cells), or U2OS (202 cells) could be seeded into 6-well plates 23.5 h prior to treatment with any one of the compounds of this invention, e.g. but not limited to Compound 1 or with AZ-67 for 23.5 h and then subjected to ionizing radiation (1.98 Gy). The inhibitors should be washed out 72 h later.

Ionizing radiation could be either y-Irradiation (from 137Cs source at a photon dose rate of 0.495 Gy/min) or X-ray high-intensity radiation. The compounds of this invention, including but not limited to Compound 1 and AZ-67 demonstrate efficacy in such model as radiosensitizers. More on the protocol can be found at “Targeting PFKFB3 radiosensitizes cancer cells and suppresses homologous recombination.” Nat Commun. 2018 Sep. 24; 9(1):3872. doi: 10.1038/s41467-018-06287-x.

### Example IX: Angiogenesis

The effect of PFKFB3 inhibitors on VEGF-A induced sprouting of HUVECs in a spheroid-based cellular angiogenesis assay. Sunitinib was used as a control. The experiments were pursued in modification of the originally published protocol (Korff and Augustin: J Cell Sci 112: 3249-58, 1999). In brief, spheroids were prepared as described (Korff and Augustin: J Cell Biol 143:1341-52, 1998) by pipetting 500 HUVEC in a hanging drop on plastic dishes to allow overnight spheroid aggregation. 50 HUVEC spheroids were then seeded in 0.9 ml of a collagen gel and pipetted into individual wells of a 24 well plate to allow polymerization. The test compounds in combination with the growth factor VEGF-A at 25 ng/ml final assay concentration) were added after 30 min by pipetting 100 μl of a 10-fold concentrated working solution on top of the polymerized gel. Plates were incubated at 37° C. for 24 hours and fixed by adding 4% Roti-Histofix (Roth, Karlsruhe, Germany). Sprouting intensity of HUVEC spheroids treated with the growth factor and the inhibitors were quantitated by an image analysis system determining the cumulative sprout length per spheroid (CSL) using an inverted microscope and the digital imaging software NIS-Elements BR 3.0 (Nikon). The mean of the cumulative sprout length of 10 randomly selected spheroids was analyzed as an individual data point.

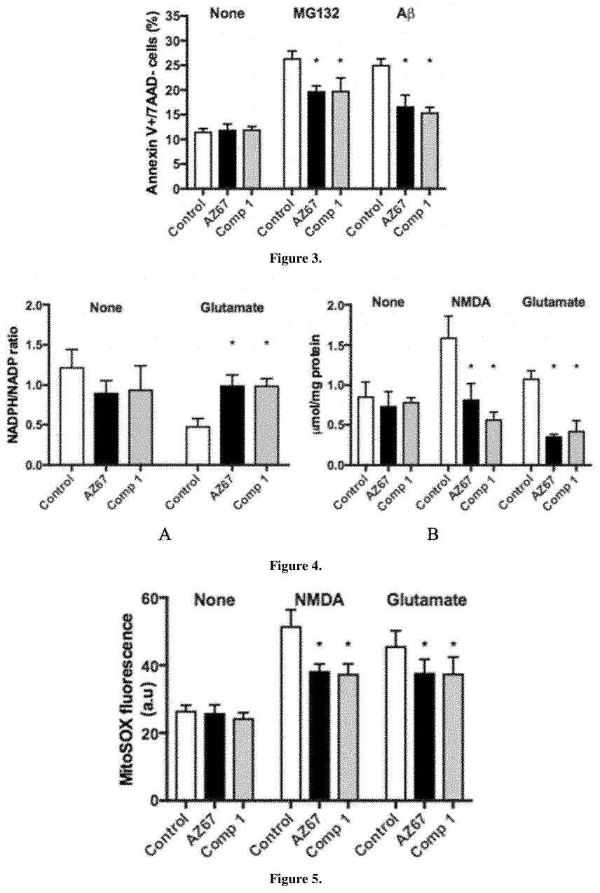

Taking into account that inhibition of PFKFB3 can result only in partial inhibition of vessel sprouting, we expected that PFKFB3 inhibitors could result in 2-3-fold reduction of total sprout length in vitro, but not totally block it. GO-672 (FIG. 3) inhibited vessel sprouting in 3D HUVEC model with EC50 20-40 μM, maximal effect was the decrease down to ˜25% at 100 μM.

Sunitinib, a positive control in this experiment, completely inhibited vessel sprouting, and its maximal effect was 100%, while GO-672 and its analogues showed maximal effect of vessel reduction to 25-50% from the initial level to 25-50% at the highest doses. Sunitinib totally inhibited vessel sprouting with EC50 0.1 μM.

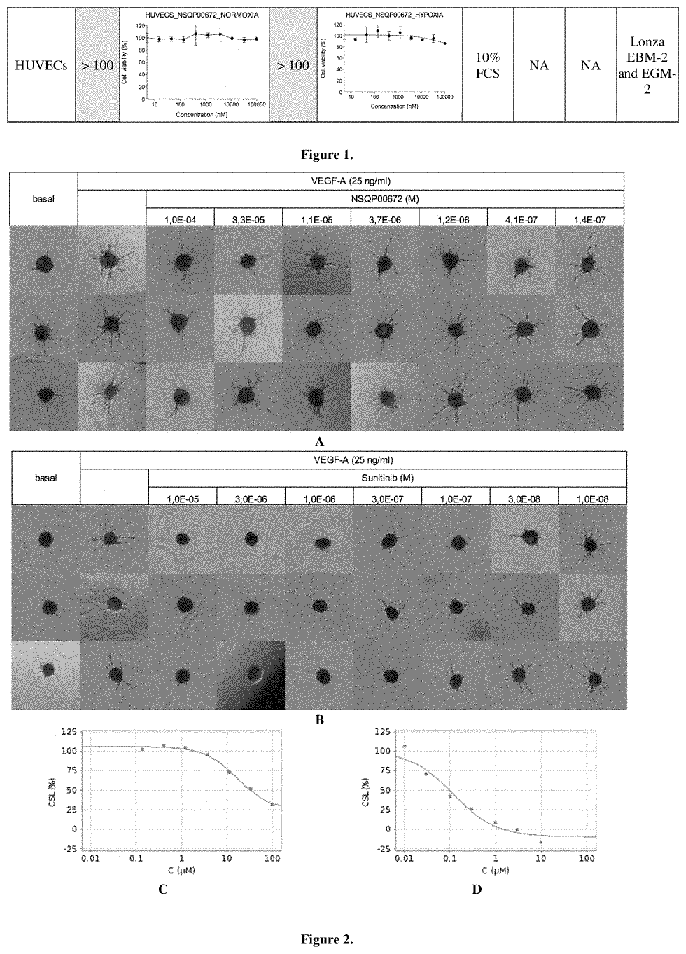

The effect of inhibition is not related to cytotoxicity, since GO-672 did not reduce cell viability of HUVECs both in normoxia and hypoxia conditions (see table HUVEC—FIG. 1).

ThermoFisher Cyquant NF cell proliferation assay kit
Normoxia conditions: 37° C., 5% CO₂, 20% O₂ incubator
Hypoxia conditions: 37° C., 5% CO₂, 1% O₂ incubator
FIG. 2. Effect of GO-672 on vessel sprouting in vitro. Raw images from the experiment with 3D spheroids of HUVEC after treatment with GO-672 (B) and Sunitinib (A). VEGF-A was used as a stimulation factor. Dose-response plot of GO-672 (C) and Sunitinib (D).

Angiogenesis (Prophetic)

An in vitro model of angiogenesis can also be used to assess the compounds disclosed herein as inhibitors of angiogenesis using system with HUVEC cell culture, e.g. but not limited to Compound 1 and AZ-67. Briefly, angiogenesis system with HUVEC cells is treated with different levels of any compound disclosed herein at 0, 0.1 μM, 1 μM, 10 μM and 100 μM and incubated for 8 hours at 37° C. and 5% CO₂. Then Calcein-AM is given to a final concentration of 2 μg/ml, and after 30 min incubation at 37° C. angiogenesis system is loaded into Acumen eX3 and scanned with appropriative instrument settings.

The compounds of this invention, including but not limited to Compound 1 and AZ-67 demonstrate efficacy as inhibitors of angiogenesis in such model.

### Example X: Protection Neurons Against Proteasome Inhibition and β-Amyloid Treatment

Since, in neurons, PFKFB3 is continuously degraded by the proteasome (Herrero-Mendez et al., 2009), we reasoned that the stabilization of PFKFB3 caused by proteasomal inhibition may trigger neuronal apoptosis.

Primary cultures of C57BL/6 mice cortical neurons were prepared from fetal animals of 14.5 days of gestation, seeded at 1.8·10⁵ cells/cm² in plastic plates coated with poly-D-lysine (10 mg/ml) and incubated in Neurobasal (Life Technologies) supplemented with 2 mM glutamine, 5 mM of glucose, 0.25 mM pyruvate and 2% B27 supplement (Life Technologies). Cells were incubated at 37° C. in a humidified 5% CO₂-containing atmosphere. At 72 hours after plating, medium was replaced using Neurobasal (Life Technologies) supplemented with 2 mM glutamine, 5 mM glucose, 0.25 mM pyruvate and 2% B27 supplement (Life Technologies) minus antioxidants (MAO; i.e., lacking vitamin E, vitamin E acetate, superoxide dismutase, catalase and glutathione). Six days after plating medium was replaced again. Neurons at 8 days in vitro were incubated with 10 mM MG132 and b-amyloid plus the PFKFB3 inhibitors for 24 hours. APC/C-conjugated annexin-V and 7-amino-actinomycin D (7-AAD) (Becton Dickinson Biosciences, BDB, San Jose, Calif., USA) were used to determine quantitatively the percentage of apoptotic neurons by flow cytometry. Cells were stained with annexin V-APC and 7-AAD, following the manufacturer's instructions, and were analysed on a FACScalibur™ flow cytometer (15 mW argon ion laser tuned at 488 nm; CellQuest software, Becton Dickinson Biosciences) using the CellQuest software (BDB). Both GFP+ and GFP− cells were analyzed separately, and the annexin V-APC-stained cells that were 7-AAD-negative were considered to be apoptotic.

As shown in FIG. 3, incubation of mouse cortical primary neurons with MG132, a widely used proteasomal inhibitor, caused neuronal apoptosis. Interestingly, this effect was significantly (by ˜45% and) prevented by AZ67 and Compound 1 (0.01 μM), indicating that a considerable proportion of the neuronal death caused by proteasomal inhibition is consequence of PFKFB3 activity. Next, we aimed to investigate if AZ67 and Compound 1 were able to protect neurons from the toxicity caused by PFKFB3 stabilization upon a different stimulus. PFKFB3 is stabilized by the stimulation of NMDA receptors (Rodriguez-Rodriguez et al., 2012), which mediate the cytotoxic actions of β-amyloid (Jarosz-Griffiths et al., 2016). Moreover, through this mechanism, β-amyloid can promote Cdh1 degradation (Fuchsberger et al. 2016). We therefore were aimed to evaluate the potential neuroprotective effect of PFKFB3 inhibition against β-amyloid-induced toxicity. As shown in FIG. 3 AZ67 and Compound 1 efficiently (by ˜60% and ˜70%, respectively) prevented the apoptotic neuronal death caused by β-amyloid treatment, indicating that a large proportion of β-amyloid neurotoxicity can be explained by neuronal PFKFB3 activity.

FIG. 3 PFKFB3 inhibitors protect neurons against MG132- and β-amyloid-induced toxicity. *-significantly different from the corresponding control (P<0.05)

### Example XI PFKFB3 Inhibitors Prevent Glycolytic Activation and Redox Stress Upon Excitotoxic Stimuli in Primary Neurons

To directly test the ability of the PFKFB3 inhibitor, AZ67 and Compound 1, to protect against the damage caused by excitotoxic stimuli, mouse primary cortical neurons were subjected to a short-term exposure to NMDA or glutamate (100 μM for 10 minutes), a well-known excitotoxic stimuli (Almeida and Bolahos, 2001). For NMDAR activation, neurons at 8 days in vitro were incubated with 100 μM glutamate (plus 10 μM glycine) or 100 μM NMDA (plus 10 μM glycine) for 10 minutes. Neurons were then washed and further incubated in culture medium with the PFKFB3 inhibitors for 24 hours. A colorimetric NADP/NADPH assay kit (Abcam) was used. Cells were resuspended in 500 μl of NADP/NADPH extraction buffer, vortexed and centrifuged at 14,000 rpm for 5 minutes to remove insoluble material. The supernatant was used for NADPH +plus NADP+ measurement. NADPH was determined in 200 μl of the supernatant, after heatinged 30 minutes at 60° C. for 30 minutes to decompose NADP+. Actual NADP and NADPH₊ concentrations were calculated by extrapolating values to a NADPH standard curve (0-100 pmol/well).

In view that this treatment causes PFKFB3 stabilization leading to glycolytic activation and PPP inhibition (Rodriguez-Rodriguez et al., 2012), we reasoned that PFKFB3 inhibition by AZ67 and Compound 1 could abrogate these effects. As shown in FIG. 4A, stimulation of glutamate receptors triggered NADPH oxidation, a hallmark of PPP inhibition (Bolaños and Almeida, 2010), an effect that was prevented by incubation with AZ67 and Compound 1. This result indicates that NADPH oxidation, upon a neuronal excitotoxic stimuli, is mostly due to PFKFB3 activation. To directly test whether this effect is associated with the control of PFKFB3 on glycolysis, we next assessed the glycolytic-end product, lactate, released to the incubation medium. As shown in FIG. 4B, both NMDA- and glutamate-mediated excitotoxic stimuli were able to activate glycolysis, and this effect was fully prevented by AZ67 and Compound 1. These data indicate that pharmacological inhibition of PFKFB3 in neurons is sufficient to prevent excitotoxic stimuli-mediated activation of glycolysis.

FIG. 4. A AZ67 and Compound 1 at 0.01 μM restore NADPH/NADP ratio under excitotoxic conditions induced by glutamate. B AZ67 and Compound 1 (at 0.01 μM) prevent the increase in lactate production promoted by excitotoxic stimuli. *-significantly different from the corresponding control (P<0.05)

Excitotoxicity is associated with increased ROS formation and redox stress, which may lead to mitochondrial fragmentation that is observed in several neurodegenerative diseases (Knott et al., 2008; Nguyen et al., 2011). Moreover, stabilization of PFKFB3 leading to increased glycolysis (FIG. 4B) is known to inhibit glucose oxidation through the PPP, which is necessary for NADPH and glutathione regeneration and, thus, for preventing redox stress in neurons (Herrero-Mendez et al., 2009; Rodriguez-Rodriguez et al., 2012). Therefore, we next investigated whether AZ67 and Compound 1 were capable of avoiding the redox stress associated with the PPP-to-glycolytic shift triggered upon excitotoxic stimuli.

Mitochondrial ROS was detected using the fluorescent probe MitoSox™ (Life Technologies). Cells were incubated with 2 μM of MitoSox™ for 30 minutes at 37° C. in a 5% CO₂ atmosphere in Hank's Balanced Salt Solution (HBSS buffer); (NaCl 134.2 mM; KCl 5.26 mM; KH₂PO₄ 0.43 mM; NaHCO₃4.09 mM; Na₂HPO₄.2H₂O 0.33 mM; glucose 5.44 mM; HEPES 20 mM; CaCl₂.2H₂O 4 mM; pH 7.4). Cells were then washed with PBS (phosphate-buffered saline, 0.1 M) and collected by smooth trypsinization. MitoSox™ fluorescence was assessed by flow cytometry and expressed in arbitrary units.

As shown in FIG. 4B, AZ67 and Compound 1 at 0.01 μM significantly prevented the increase in mitochondrial ROS triggered both by the NMDA and glutamate excitotoxic models in primary neurons.

FIG. 5. AZ67 and Compound 1 (0.01 μM) prevent mitochondrial ROS increase in a excitotoxic model in mouse primary cortical neurons. *-significantly different from the corresponding control (P<0.05)

### Example XII: PFKFB3 Inhibitors Prevent Neuronal Death Upon Excitotoxic Stimuli by Activating Glycolysis

To show this, neurons were treated with glutamate of NMDA, and apoptosis assessed by annexin V+/7AAD− staining using flow cytometry, as described above.

As shown in FIG. 5, both glutamate and NMDA significantly increased apoptotic neuronal death, an effect that was significantly (by 76% and 90%, respectively) prevented by AZ67 (0.01 μM) and by Compound 1 (by 70% for both stimuli). To address whether the protection caused by PFKFB3 inhibitors was a consequence of hampering the glycolytic activation (FIG. 4B), we aimed to investigate whether the overexpression of the glycolytic enzyme, PFK1-muscle isoform, could revert PFKFB3 inhibitors-mediated neuroprotection. We focused on the muscle PFK1 isoform given its very low sensitivity to F-2,6-BP allosteric activation (Vora et al., 1985) and hence its independence on PFKFB3 levels to fully activate glycolysis (Almeida et al., 2010). Accordingly, neurons were first transfected with the full-length cDNA encoding for PFK1-M, and then subjected to the excitotoxic insult. Cells were transfected with 1.6 μg/mL of a pIRES2-EGFP plasmid vector (Invitrogen) harbouring a full-length cDNA coding for the human muscle 6-phosphofructo-1-kinase muscle isoform (PFK1-M) (accession number, NM_000289.1) using Lipofectamine LTX-PLUS Reagent (Life Technologies) according with manufacturer's protocol. Transfections were performed 24 hours before cells collection. Control cells were transfected with the empty vector. Cells were treated with excitotoxic stimuli as described above.

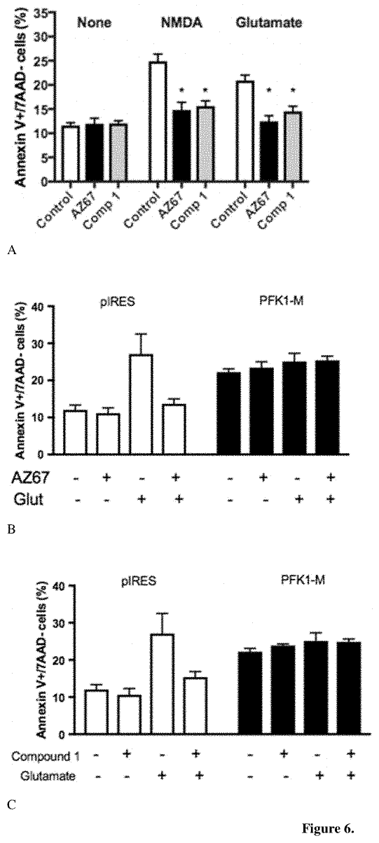

As shown in FIG. 6B-C, PFK1-M overexpression was able to abolish the neuroprotection caused by AZ67 and Compound 1. These results indicate that the neuroprotection exerted by this compound is due to its ability to prevent glycolytic activation.

FIG. 6. (A) AZ67 and Compound 1 protect mouse primary cortical neurons from apoptosis induced by NMDA and glutamate. Expression of PFK1 (muscle isoform or PFK1-M) abrogates the neuroprotective effect of AZ67 (B) and Compound 1 (C).

### Example XIII: Efficacy of PFKFB3 Inhibitors in Treatment of Ischemic Stroke

The neuroprotective effect of compounds (PFKFB3 inhibitors) may be evaluated in an animal model of ischemic stroke. Stroke is the most common fatal neurological disease and the majority of strokes are ischemic strokes, i.e. those that result from the blockage of blood vessels in the brain. The middle carotid artery occlusion (MCAO) protocol is often used to model permanent (24 h) or transient (shorter times periods such as 30 min, 1 h, or 2 h followed by reperfusion) occlusion (1,2). The preferred protocol is the transient occlusion protocol, and the compounds after administered immediately after the ischemia to best resemble the clinical situation. Physiological tests and infarct volume measurements can then be performed to evaluate the effect of the tested compounds.

Middle Cerebral Arterial Occlusion (MCAO).

For the MCAO model of brain ischemia, 10 weeks-old male C57BL/6-J mice were used (n=8 animals per condition) following a published protocol (1,2). Mice were anesthetized with 4% (vol/vol) Sevoflurane in a mixture of ⅓ O2 and ⅔ N2O using a vaporizer. After the induction of anesthesia, Sevoflurane was reduced to 3% (vol/vol). The body temperature was maintained at 36.5° C. during the surgery. A laser-Doppler flow probe attached to a flowmeter was located over the thinned skull in the MCAO area (4 mm lateral to bregma) to monitor the relative cerebral brain flow during the experiment. Under operating microscope, the common, external and internal carotid arteries were dissected from connective tissue through a midline neck incision. The common carotid artery (CCA) was carefully dissected from the surrounding nerves with caution to avoid harming of the vagal nerve. A suture monofilament was introduced from the lumen of the external carotid artery (ECA) into the internal carotid artery (ICA) to a distance of 9-10 mm beyond the CCA bifurcation, in order to occlude the origin of middle cerebral artery (MCA). The filament was removed 30 mins after occlusion. AZ67 or compound 1 (60 mg/kg of body weight) or vehicle were administered in a bolus (200 μl) via the jugular vein immediately after reperfusion. The incision on the neck was then sutured and the mice placed in a 35° C. nursing box until recovery from anesthesia (5-10 mins), when they were returned to the cages.

Rota-Rod Analysis.

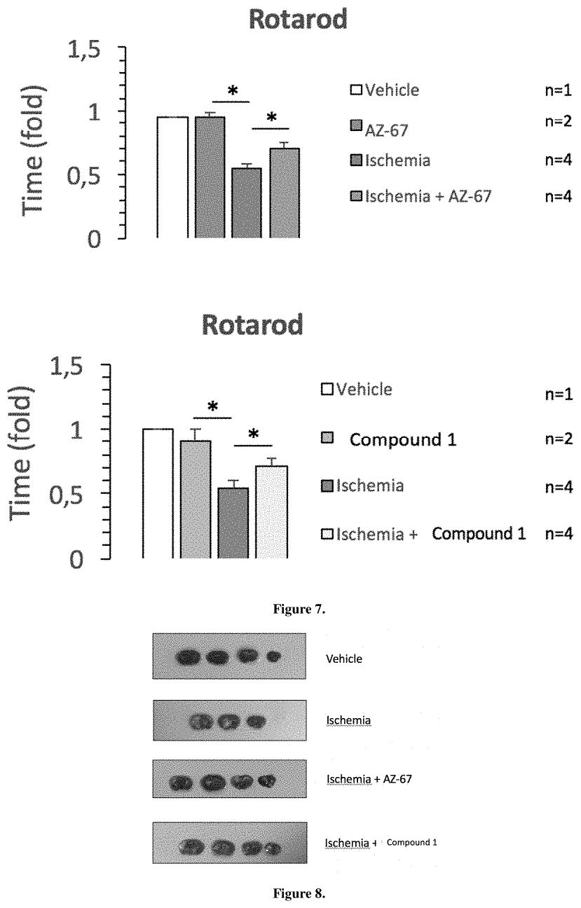

An accelerating rotarod test was used to determine motor coordination. Animals were trained during the immediate three previous days of the MCAO surgery. The first day, mice stayed on the rotating rod at a constant speed of 4 rpm, and the remaining 2nd and 3rd day they stayed at an accelerating speed (4 to 40 rpm in 5 mins). For the test, which was performed 24 hours after the MCAO surgery, mice were subjected to three consecutive trials at the accelerating speed for 5 mins (at 15 mins intervals). The latency to fall was determined and expressed in seconds. AZ67 and compound 1 showed almost 50% improvement in rota-rod test performance (FIG. 7).

FIG. 7. Evaluation of effect of PFKFB3 inhibitors on motor coordination after MCAO using rota-rod test. *: p≤0.05 versus ischemia (ischemia=ischemia followed by re-perfusion)

Infarct Volume.

Immediately after the rota-rod test, mice were euthanized by cervical dislocation after CO₂ overdose, and the brain extracted and sliced in 2-mm coronal sections with a brain matrix on ice, which were used to determine the infarct volume after incubation of the slices in 2% (wt/vol) 2,3,5-triphenyltetrazolium chloride in phosphate-buffered saline (136 mM NaCl, 27 mM KCl, 7.8 mM Na2HPO4, 1.7 mM KH2PO4, pH 7.4) for 20 minutes at room temperature. Pictures of the brain sections were taken and the images processed using the using the NIH image-processing package ImageJ 1.43n. Infarct volumes were determined by multiplying the selected infarcted area by the width of the slices. In order to correct the infarct volume by the edema, the ratio lesion volume of the ipsilateral (affected) versus that of the contralateral (unaffected) hemispheres was calculated. The percentage of infarct volume was calculated using the following formula:

$$\% Infarct Volume = \frac{Infarcted volume corrected by edema}{Infarcted hemisphere volume} \times 100$$

Infarct volume in mice treated with PFKFB3 inhibitors was about 20-25% lower than in vehicle treated animals (FIG. 7).

FIG. 8 Evaluation of effect of PFKFB3 inhibitors on infarct volume of mice after MCAO.

### REFERENCES

1. Engel, O., Kolodziej, S., Dirnagl, U., Prinz, V. Modeling Stroke in Mice—Middle Cerebral Artery Occlusion with the Filament Model. J. Vis. Exp. (47), e2423, doi:10.3791/2423 (2011).
2. Chiang, T., Messing, R. O., Chou, W. Mouse Model of Middle Cerebral Artery Occlusion. J. Vis. Exp. (48), e2761, doi:10.3791/2761 (2011).

In neuroprotection examples it is worth noticing that to the best of our knowledge the binding mode of compounds CHEMBL3422676 (AZ67), (2S)—N-(4-{[3-cyano-1-(2-methylpropyl)-1H-indol-5-yl]oxy}phenyl)pyrrolidine-2-carboxamide (AZ-26) and similar compounds from the article doi 10.1021/acs.jmedchem.5b00352 differs from the mode of the compound 4-({4-carboxy-4′-fluoro-[1,1′-biphenyl]-3-yl}carbamoyl)benzene-1,3-dicarboxylic acid (GO-0003583) and similar compounds. The pose of CHEMBL3422676 (AZ67) is close to Asn163, the pose of GO-0003583 is close to Arg74. Despite the difference in binding pose, these compounds cause same biological effect-neuroprotection and others as described in this disclosure.

### Example XIV: Anti-Aging Treatment (Prophetic)

Male C57BL/6J mice aged 10 months can be used for this study, 50 mice can be assigned to each group. Mice from different groups can be treated with AZ67 or Compound 1 (or as other example in separate group with any other PFKFB3 cell permeable small molecule inhibitor preferably with at dose 30 mg/kg, 60 mg/kg, 120 mg/kg, or vehicle daily via i.p. route during 4 weeks, in another group—during 8 weeks, in another group—during 6 months, in yet another group—during 1 year.

Four weeks after treatment start, a standard blood count analysis can be performed and the biological age is estimated. Biological age should be decreased in AZ67 and Compound 1 treatment groups compared to vehicle control.

The biological age model is a form a physiological frailty index adopted from [Antoch M P, Wrobel M, Kuropatwinski K K, Gitlin I, Leonova K I, Toshkov I, Gleiberman A S, Hutson A D, Chernova O B, Gudkov A V. Physiological frailty index (PFI): quantitative in-life estimate of individual biological age in mice. Aging (Albany N.Y.). 2017 Mar. 19; 9(3):615-626] and using the subset of the measurements, the hemo analysator readouts.

The bioage-calculation procedure consists of the following stages:

1) subtract the reference mean value (column MEAN in the table) of each test;

2) multiply by the coefficient from column COEF;

| ID | MEAN | COEF |
|---|---|---|
| HB (g/dL) | 14.7810810811 | −0.324994418476 |
| LY (K/uL) | 6.78821787942 | −0.0403357974256 |
| MCH (Pg) | 15.2156964657 | −0.305640352983 |
| MCHC (g/dL) | 33.18497921 | 0.0243410007583 |
| MCV(fL) | 45.8556652807 | −0.071912079313 |
| MO (K/uL) | 0.187391325364 | 2.99337099222 |
| MPV (fl) | 5.82976611227 | −0.0622717180147 |
| PLT (k/ul) | 1258.6456341 | 0.00122980926892 |
| RBC (M/uL) | 9.74016632017 | −0.227470069201 |
| WBC (K/uL) | 8.83614345114 | 0.0437124309324 |

3) sum the resulting values.

The performance of the biological age was independently confirmed by the correlation between the average biological age in cohorts of mice belonging to strains at any given age and the remaining lifespans. We used the data from the publicly available data on hematological phenotypes of mice from (Peters LL, Cheever E M, Ellis H R, Magnani P A, Svenson K L, Von Smith R, Bogue M A. Large-scale, high-throughput screening for coagulation and hematologic phenotypes in mice. Physiol Genomics. 2002 Dec. 3; 11(3):185-93), and found, that the proposed biological age is significantly associated with longevity (the correlation of the biological age to lifespan, p-val=4E-6, the biological age detrended by the chronological age to lifespan, p-val=0.015 for male mice).

A larger biological age value, therefore, corresponds to a shorter lifespan and the other way around. The reduction of bioage would imply that the animal is rejuvenated to some extend and healthspan and lifespan expectancy is increased. Therefore the intervention that lead to this effect is expected to have an anti-aging treatment potential.

Effect of the treatment on biological age and frailty index may be estimated by many methods, including but not limited to—based on changes in standard blood count [Gudkov], DNA methylation [Stubbs™, Bonder M J, Stark A K, Krueger F; B I Ageing Clock Team, von Meyenn F, Stegle O, Reik W. Multi-tissue DNA methylation age predictor in mouse. Genome Biol. 2017 Apr. 11; 18(1):68; Horvath S. DNA methylation age of human tissues and cell types. Genome Biol. 2013; 14(10):R115], lifespan [Harrison D E, Strong R, Sharp Z D, Nelson J F, Astle C M, Flurkey K, Nadon N L, Wilkinson J E, Frenkel K, Carter C S,

Pahor M, Javors M A, Fernandez E, Miller R A. Rapamycin fed late in life extends lifespan in genetically heterogeneous mice. Nature. 2009 Jul. 16; 460(7253):392-5], and healthspan assessed by frailty index. A frailty index is created by counting the accumulation of deficits in health across many systems in the body. Deficits measured to construct a frailty index include a large number of health-related variables related to the function of systems that are known to change with age in both human and animal models [Parks R J, Fares E, Macdonald J K, Ernst M C, Sinal C J, Rockwood K, Howlett S E: A procedure for creating a frailty index based on deficit accumulation in aging mice. J Gerontol A Biol Sci Med Sci 2012; 67:217-227.]. These variables provide information about the following: (a) activity, including distance moved, velocity of movement and rearing frequency; (b) hemodynamic status, including heart rate, systolic and diastolic blood pressure; (c) body composition, including body mineral content, percent body fat and percent lean tissue; and (d) basic metabolism and organ function, including serum electrolyte levels, hematocrit and urea levels clinical signs, symptoms, diseases, and laboratory and radiographic abnormalities.

Another way to test the anti-aging effect is after the same as described administration to evaluate healthspan and lifespan of mice in treated by PFKFB3 inhibitor and treated by vehicle groups, the median healthspan and lifespan of mice treated with PFKFB3 inhibitor should be greater.

### Example XV: Anti-Aging Gene Therapy Treatment (Prophetic)

Male C57BL/6J mice aged 10 months can be used for this study, 50 mice can be assigned to each group. Mice can be housed at the specific pathogen-free animal house by the methods known in the art. Mice in treatment group can be treated with the AAV vector containing shRNA or CRISPR/CAS9 cassette. Vectors can be administered via tail vein injection at a concentration of 3.5 E12 viral genomes per mouse. Viral vectors can be acquired ready for use or generated and purified by methods know in the art. Vectors can be produced through triple transfection of HEK293T. Expression cassettes can be under the control of the cytomegalovirus promoter and can contain an SV40 polyA signal for EGFP and the cytomegalovirus promoter. AAV particles can be purified using 2 cesium chloride gradients, dialyzed against phosphate buffered saline (PBS) and filtered. Viral genome particle titers can be determined by a quantitative real-time polymerase chain reaction (PCR) method. For liver delivery liver-specific serotype of adenovirus AAV8 may be used or any other delivery platform or tool. The biological age, aging related biomarkers and endpoint can be checked as shown in Example XIV above or by other methods known in the art.

### Example XVI: Example of Composition (Prophetic)

Exemplary Injection Formulation Containing an agent of this disclosure. The vial contains 5 mg of PFKFB3 inhibitor, e.g. AZ67 or Compound 1-50 mg as a powder for injection. Powder for injection is to be reconstituted with sterile water for injections and further diluted in 0.9% sodium chloride solution for infusion. After reconstitution, each vial contains substance for injection. Inactive ingredients: sodium phosphate monobasic monohydrate, sodium phosphate dibasic dihydrate, sucrose and polysorbate 80.

### Example XVII: Example of Composition (Prophetic)

Exemplary Injection Formulation Containing an agent of this disclosure. The vial contains 5 mg of PFKFB3 inhibitor. Powder for injection is to be reconstituted with 5% DMSO, 95% Captisol (30% in water) in case of AZ67 and 20% (v/v) PEG400 in phosphate buffered saline (pH:8.0±0.2) in case of Compound 1 After reconstitution, each vial contains substance for injection.

### Example XVIII: Injection Formulation (Prophetic)

AZ67 in a sterile form for reconstitution as a suspension for subcutaneous injection or reconstitution as a solution with further dilution for intravenous infusion. Vials of az67 contain 100 mg of AZ67 and 100 mg of mannitol as a sterile lyophilized powder.

### Example XIX: Tablet Formulation (Prophetic)

Compound CHEMBL3422651-75 mg, Ludipress −100 mg, Kollidon CL −10 mg, Magnesium stearate −10 mg, Aerosil −5 mg.

Compound 1 −75 mg, Ludipress −100 mg, Kollidon CL −10 mg, Magnesium stearate −10 mg, Aerosil −5 mg.

### Example XX: Tablet Formulation (Prophetic)

Orally bioavailable form of small molecule PFKFB3 inhibitor AZ67 −75 mg, Ludipress −100 mg, Kollidon CL −10 mg, Magnesium stearate −10 mg, Aerosil −5 mg.

Orally bioavailable form of small molecule PFKFB3 inhibitor-Compound 1, Ludipress −100 mg, Kollidon CL −10 mg, Magnesium stearate −10 mg, Aerosil −5 mg.

### Example XXI: Kit with Anti-Aging Therapy (Prophetic)

Plastic box, comprising at least one pharmaceutical composition of this disclosure, and paper instruction, wherein amongst other the following wording is present: “This PFKFB3 inhibitor is indicated for: anti-aging treatment, rejuvenation, frailty treatment, amelioration of moderate cognitive decline, amelioration hand grip strength lose and amelioration of other age related deficits”.

or “This AZ67 compound is a PFKFB3 inhibitor and is indicated for: anti-aging treatment, rejuvenation, frailty treatment, amelioration of moderate cognitive decline, amelioration hand grip strength lose and amelioration of other age related deficits”.

or “This 4-({4-carboxy-2′,4′-dichloro-[1,1′-biphenyl]-3-yl}carbamoyl)-6-hydroxybenzene-1,3-dicarboxylic acid is a PFKFB3 inhibitor and is indicated for: anti-aging treatment, rejuvenation, frailty treatment, amelioration of moderate cognitive decline, amelioration hand grip strength lose and amelioration of other age related deficits”.

### Example XXII: Kit (Prophetic)

Plastic box, comprising at least one pharmaceutical composition of this disclosure, and paper instruction, wherein amongst other the following wording is present:

“This small molecule PFKFB3 inhibitor (name of chemical) is indicated for neuroprotection” or

“This AZ67 compound is a PFKFB3 inhibitor and is indicated for neuroprotection”.

or 4-({4-carboxy-2′,4′-dichloro-[1,1′-biphenyl]-3-yl}carbamoyl)-6-hydroxybenzene-1,3-dicarboxylic acid is a PFKFB3 inhibitor and is indicated for neuroprotection″.

### Example XXIII: Neuroprotection

For NMDAR activation, neurons at 8 days in vitro were incubated 100 μM NMDA (plus 10 μM glycine) for 10 minutes. Neurons were then washed and further incubated in culture medium with the PFKFB3 inhibitors for 24 hours. For b-amyloid treatment, neurons at 8 days in vitro were incubated with 10 mM b-amyloid plus the PFKFB3 inhibitors for 24 hours. The Protection factor was estimated as [AMC(beta-A)−AMC(DMSO)]/[AMC(beta-A)−AMC(Test article)] or [AMC(beta-A)−AMC(DMSO)]/[AMC(NMDA)−AMC(Test article)], where:

AMC(DMSO)—% dead neuron in case of DMSO treatment

AMC(beta—A)—% dead neuron in case of b—amyloid treatment

AMC(NMDA)—% dead neuron in case of NMDA treatment

AMC(Test article)—% dead neuron in case of PFKFB3 inhibitors treatment.

The results are presented in the table:

| | Protection | |
|---|---|---|
| Compound | factors | Treatment |
| 2-{4-carboxy-2′,4′-dichloro-[1,1′-biphenyl]-3-yl}-1,3-dioxo-2,3-dihydro-1H-isoindole-5-carboxylic | 1.75 | beta-amyloid |
| acid | | |
| 3-[5-chloro-1,3-dioxo-6-(2H-1,2,3-triazol-4-yl)-2,3-dihydro-1H-isoindol-2-yl]-3′,4′-difluoro-[1,1′- | 1.15 | beta-amyloid |
| biphenyl]-4-carboxylic acid | | |
| 2-{4-carboxy-3′,4′-difluoro-[1,1′-biphenyl]-3-yl]-6-hydroxy-1,3-dioxo-2,3-dihydro-1H-isoindole-5- | 2.15 | beta-amyloid |
| carboxylic acid | | |
| 4-({4-carboxy-2′,4′-dichloro-[1,1′-biphenyl]-3-yl}carbamoyl)-6-hydroxybenzene-1,3-dicarboxylic | 8.07 | beta-amyloid |
| acid | | |
| 4-{[2-carboxy-5-(4,4-dimethylpiperidin-1-yl)phenyl]carbamoyl}-6-hydroxybenzene-1,3- | 4.33 | beta-amyloid |
| dicarboxylic acid | | |
| 2-{[2-carboxy-5-(4,4-dimethylcyclohexyl)phenyl]carbamoyl}-5-hydroxybenzene-1,4-dicarboxylic | 2.05 | beta-amyloid |
| acid | | |
| 2-[(2′,4′-dichloro-4-{[2-(dimethylamino)ethoxy]carbonyl}-[1,1′-biphenyl]-3-yl)carbamoyl]-5- | 1.43 | beta-amyloid |
| {[dimethyl(oxo)-λ⁶-sulfanylidene]carbarnoyl}benzoic acid | | |
| (2S)-N-[4-({3-cyano-1-[(3,5-dimethyl-1,2-oxazol-4-yl)methyl]-1H-indol-5- | 1.68 | beta-amyloid |
| yl}oxy)phenyl]pyrrolidine-2-carboxamide | | |
| ethyl 3′,4′-difluoro-3-[1-oxo-6-(2H-1,2,3-triazol-4-yl)-2,3-dihydro-1H-isoindol-2-yl]-[1,1′-biphenyl]- | 2.35 | beta-amyloid |
| 4-carboxylate | | |
| 3′,4′-difluoro-3-[1-oxo-6-(2H-1,2,3-triazol-4-yl)-2,3-dihydro-1H-isoindol-2-yl]-[1,1′-biphenyl]-4- | 12.17 | beta-amyloid |
| carboxylic acid | | |
| propan-2-yl 3′,4′-difluoro-3-[1-oxo-6-(2H-1,2,3-triazol-4-yl)-2,3-dihydro-1H-isoindol-2-yl]-[1,1′- | 1.43 | beta-amyloid |
| biphenyl]-4-carboxylate | | |
| (2S)-N-(4-{[3-cyano-1-(2-methylpropyl)-1H-indol-5-yl]oxy]phenyl)pyrrolidine-2-carboxamide | 1.97 | beta-amyloid |
| 1-(dimethylamino)propan-2-yl 3′,4′-difluoro-3-[1-oxo-6-(2H-1,2,3-triazol-4-yl)-2,3-dihydro-1H- | 2.56 | NMDA |
| isoindol-2-yl]-[1,1′-biphenyl]-4-carboxylate | | |
| (2-oxo-1,3-oxazolidin-5-yl)methyl 3′,4′-difluoro-3-[1-oxo-6-(2H-1,2,3-triazol-4-yl)-2,3-dihydro-1H- | 1.05 | NMDA |
| isoindol-2-yl]-[1,1′-biphenyl]-4-carboxylate | | |
| 2,3-dihydroxypropyl 3′,4′-difluoro-3-[1-oxo-6-(2H-1,2,3-triazol-4-yl)-2,3-dihydro-1H-isoindol-2-yl]- | 1.35 | NMDA |
| [1,1′-biphenyl]-4-carboxylate | | |

### Example XXIV: Syngeneic Subcutaneous (s.c.) Melanoma Model Bi16F10

The objective of the study was to evaluate the in vivo anti-tumor efficacy of 4-({4-carboxy-2′,4′-dichloro-[1,1′-biphenyl]-3-yl}carbamoyl)-6-hydroxybenzene-1,3-dicarboxylic acid (Compound 1, GO-672) (as a single agent and in combination with cisplatin) in the s.c. B16-F10 murine melanoma model in C57BL/6 female mice.

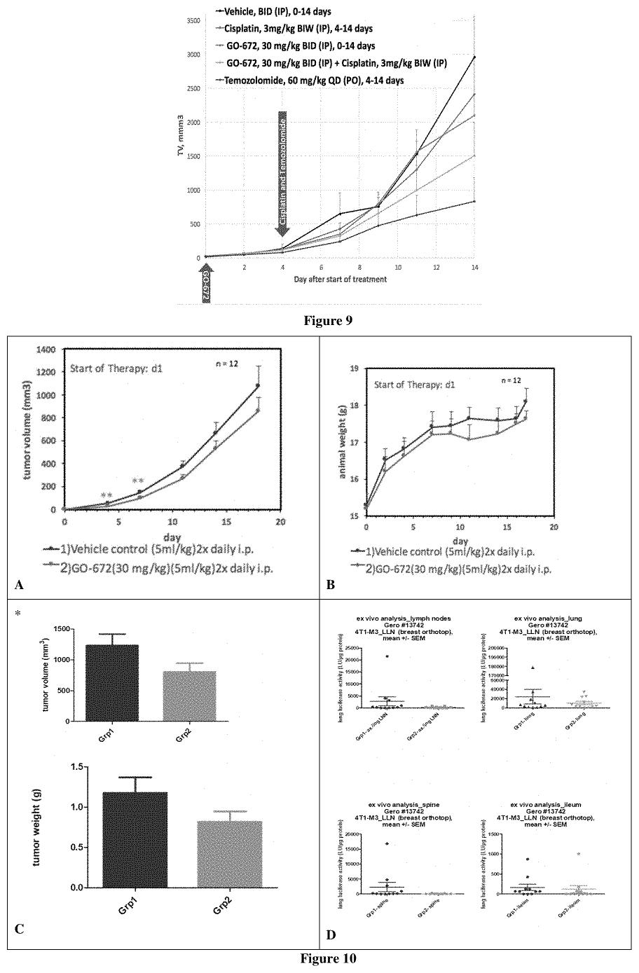

Each mouse was inoculated subcutaneously at the right lower flank with B16-F10 tumor cells (2×10\hat{ }5/mouse) in 0.1 ml of serum-free RPMI-1640 for tumor development. Compound 1 (i.p. 30 mg/kg once daily and i.p. 30 mg/kg twice daily) and vehicle treatment were started at day 10 post inoculation (average tumor volume (TV) 30 mm3). Cisplatin (i.p. 3 mg/kg twice a week) and Temozolomide (p.o. 60 mg/kg daily) treatment were initiated 3 days later (average TV 160 mm3). Due to highly aggressive growth of B16-F10 model animals with TV >50 mm3 at the beginning of treatment with Compound 1 were excluded from analysis. The regimen of 30 mg/kg Compound 1 QD was not effective both in mono and combo groups (data not shown), while the 30 mg/kg twice daily regimen resulted in a moderate effect in combination with cisplatin, which was higher than cisplatin alone, but lower if compared with the positive control—Temozolomide (FIG. 9).

FIG. 9. Effect of GO-672 as a single agent and in combination with cisplatin in syngeneic s.c. B16-F10 model. Data are presented as mean+SEM (N=5-7 on Day 0 and 3-6 on Day 14).

### Example XXV: Orthotopic 4T1-M3-Luc Syngeneic Model

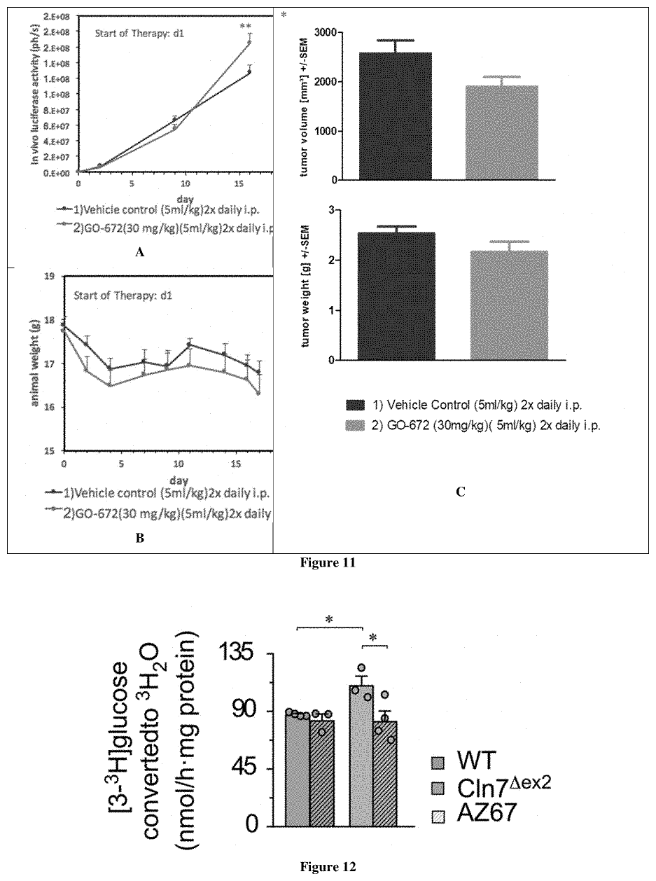

The aim of this study was to evaluate the antitumor and anti-metastatic efficacy of GO-672 (4-({4-carboxy-2′,4′-dichloro-[1,1′-biphenyl]-3-yl}carbamoyl)-6-hydroxybenzene-1,3-dicarboxylic acid) (30 mg/kg twice daily, i.p.) in an orthotopic 4T1-M3-Luc syngeneic breast tumor model in female BALB/c mice in vivo. Results are presented in FIG. 10. Treatment with GO-672 resulted in a small reduction of tumor volume only on days 4 and 7 (FIG. 10A), the T/C ratio was 47% and 65% (P<0.01, one-tailed Mann-Whitney U Test). No significant body weight loss was observed in the treatment groups, reduction of body weight in GO-672 group vs control group was not significant (FIG. 10B).

After necropsy on day 18 tumor volume but not tumor weight in GO-672 group was significantly lower in comparison with the vehicle group (P<0.05), (FIG. 10C). Level of metastasis was evaluated in lymph nodes, lung, spine and ileum by ex vivo luciferase bioluminescence measurements (FIG. 10D). No statistically significant difference in level of metastasis was found between the vehicle group and the treated group.

FIG. 10. Effect of GO-672, 30 mg/kg twice daily (pink) in orthotopic 4T1-M3-Luc model in comparison with vehicle (blue) in female BALB/c mice (N=12 on day 0). (A) Tumor volume and (B) animal weight. (C) Tumor volume and weight after necropsy (N=11). (D) Ex vivo metastasis evaluation based on luciferase bioluminescence (N=11). Data are presented as mean+SEM (A-C) and mean±SEM (D). * and ** are significant difference (P<0.05 and P<0.01, correspondingly) in comparison with the vehicle group, one-tailed Mann-Whitney U Test.

### Example XXVI: Syngeneic Orthotopic RENCA Model

The aim of this study was to evaluate the antitumor efficacy of GO-672 (4-({4-carboxy-2′,4′-dichloro-[1,1′-biphenyl]-3-yl}carbamoyl)-6-hydroxybenzene-1,3-dicarboxylic acid) (30 mg/kg twice daily, i.p.) in an orthotopic RENCA syngeneic renal tumor model in female BALB/c mice in vivo. Results are presented in FIG. 11.

Treatment with GO-672 resulted in an increase of bioluminescence on day 16 (FIG. 11A), but after necropsy on day 17 tumor volume in GO-672 group was significantly decreased in comparison with the vehicle group (P<0.05), while no statistically significant difference in tumor weight was found after GO-672 treatment (FIG. 11C).

FIG. 11. Effect of GO-672, 30 mg/kg twice daily (pink) in orthotopic RENCA model in comparison with vehicle (blue) in female BALB/c mice. (A) Tumor volume and (B) animal weight. (C) Tumor volume and weight after necropsy (N=10-12). Data are presented as mean+SEM. * and ** are significant difference (P<0.05 and P<0.01 respectively) in comparison with vehicle group, one-tailed Mann-Whitney U Test.

### Example XXVII: Traumatic Brain Injury Treatment (TBI) (Prophetic)

Neuroprotective effect of PFKFB3 inhibitors may be evaluated in a mouse model of traumatic brain injury (TBI). 10-12 weeks old C57BL/J male mice are used for this study, 15 animals per study group. Controlled cortical impact (CCI) technique may be used for mouse TBI model as described in (Romine et al, 2014). Briefly, mice are anesthetized and craniectomy is performed. Next, the impact is made using an impact system consisting of control box to set impact parameters, an actuator to perform the impaction, and a stereotactic frame to secure the actuator and mouse head for impact. Deformation depths of 0.5-1.0 mm is used to induce moderate TBI. Mouse is placed on a warm pad to maintain body temperature, and once bleeding has stopped, the wound is sutured. Then, the animal is placed back into the cage on the warm pad to recover. Any one of PFKFB3 inhibitors, e.g. Compound 1 or AZ67 at dosis 60 mg/kg is administered via IV route daily during 7 days starting 1 hour after trauma.

Seven days after the injury the neuroprotective effect of PFKFB3 inhibitors can be evaluated by comparing animal performance in physiological test, for example, accelerating rotarod test, as described above. Histology of brain sections is performed. The compounds of this invention, including but not limited to Compound 1 and AZ-67 demonstrate efficacy as neuroprotectors in such model.

### Example XXVIII Improving T-Cell Expansion and Function for Adoptive T-Cell Therapy Using Ex Vivo Treatment with PI3Kd Inhibitors (Prophetic)

Frozen PBMCs could be thawed and rested overnight at 37° C. in RPMI 1640 supplemented with 10.54% fetal bovine serum, 99.54 U/mL penicillin, 99.53 mg/mL streptomycin, and 49.87 mM 2-mercaptoethanol. Cells should be then cultured in 96-well flat-bottom plates at 2.04E+6 cells/mL in complete media containing 29.95 U/mL interleukin-2 and anti-CD3/CD28 beads at a 1:1 bead to cell ratio. Compound 1 or AZ67 or any PFKFB3 mentioned in this document could be added at culture initiation, and VIPhyb should be added daily. The final DMSO concentration should be 0.1% in all wells. Cells could be counted and subcultured on day 7 with the addition of fresh beads, IL-2, and compounds. The cells could be then allowed to expand for an additional 3 or 7 days. Cells could be counted and phenotyped on day 7 and on day 10 or 14.

Subsets of T cells from heavily treated DLBCL patients could be sorted according to expression of CD27 and CD28 using a BD FACS Aria II. Two populations could be sorted: T cells lacking expression of both CD27 and CD28 and the remaining cells (CD27+CD28− CD27−CD28+, and CD27+CD28+). Gating strategies for cell sorting excluded other blood cells, including granulocytes, monocytes, natural killer cells, dendritic cells, and B cells. Three populations could be expanded separately in culture under the conditions described above: the total T cell population including all subsets, the CD27−CD28− population, and the mixed population that won't contain CD27−CD28− cells. The cells could be expanded for a period of 14 days whereupon they were analyzed for viability, total cell counts, and expression of surface markers.

On the final day of expansion, the cells could be washed, counted, and resuspended in sterile PBS. 2.95E+6 cells could be injected intravenously into NSG mice via the lateral tail vein. 14 days following adoptive transfer, blood could be collected and the frequencies, absolute numbers, and phenotypes of persisting human T cells could be determined by flow cytometry using CD45 APC and CD3 PE-CF594.

Compound 1 or AZ67 or any PFKFB3 mentioned in this document can increase yield of T cells with a less differentiated phenotype following ex vivo expansion. The addition of VIPhyb during T-cell expansion further could increase the frequency of CD27−CD28− T cells. The ex vivo cytotoxicity and in vivo antitumor activity of T cells treated with PFKFB3 inhibitors, VIPhyb, or a combination should be significantly greater than that of T cells from control cultures.

### Example XXIX Cytotoxicity

Cells were collected and plated into 96-well white tissue culture plates (Corning, Cat. No. 3610) up to the final volume 100 μl. Two sets of plates were seeded for normoxia (20% O2), and hypoxia (1\% O2). Four extra plates were seeded for T_0 and T_{72} (normoxia and hypoxia), with the same seeding densities and serum conditions. All cells were incubated in the plates overnight at 37° C., 5% CO₂ incubator.

TABLE EXXIX-1
Results of in vitro cytotoxicity of Compound 1 under various
conditions in human cell lines (72 h of incubation)

| | IC₅₀ | | | | |
|---|---|---|---|---|---|
| Cell line | Norm² | Hyp³ | Albumin | Glucose, mM | Medium |
| A549 | >100 | >100¹ | 1% FBS | 20 | DMEM |
| MDA-MB-231 | >100 | >100 | 10% FBS | — | Leibovitz's L-15 |
| MDA-MB-231 | >100 | >100 | 10% FCS | 5.5 | DMEM⁴ |
| T47D | 7.6 | 100 | 1% FBS | 20 | DMEM + 0.2 |
| | | | | | Units/ml bovine |
| | | | | | insulin |
| T47D | >100 | >100¹ | 10% FCS | 5.5 | DMEM |
| U87MG | >100 | >100 | 10% FBS | 5.5 | EMEM |
| MCF7 | >100 | >100¹ | 10% FCS | 5.5 | DMEM |
| HCC1806 | >100 | >100 | 10% FCS | 5.5 | DMEM |
| HUVECs | >100 | >100 | 10% FCS | NA | Lonza EBM-2 and |
| | | | | | EGM-2 |
| RENCA | >100 | >100 | 1% FBS | 20 | DMEM |
| 4T1 | 95 | >100 | 1% FBS | 5.5 | RPMI-1640 |
| B16-F10 | >100 | >100 | 10% FBS | 20 | DMEM |

### Example XXX Combination with Anticancer Agents

| | | IC50 of | | |
|---|---|---|---|---|
| | anticancer | anticancer | IC50 of | Cell |
| Compound | agent | agent, uM | combination, uM | line |
| 3-[3-methyl-5-(propan-2-yl)-1- | Paclitaxel | 0.01 | 0.00665 | MDA-MB-468 |
| benzothiophene-2- | | | | |
| sulfonamido]benzoic acid (qpd- | | | | |
| 14071) 2 uM | | | | |
| 3-[3-methyl-5-(propan-2-yl)-1- | Doxorubicin | 0.07 | 0.045 | MDA-MB-468 |
| benzothiophene-2- | | | | |
| sulfonamido]benzoic acid (qpd- | | | | |
| 14071) 2 uM | | | | |
| 4-({4-carboxy-3′,4′-difluoro-[1,1′- | Paclitaxel | 0.01 | 0.0071 | MDA-MB-468 |
| biphenyl]-3-yl}carbamoyl)-6- | | | | |
| hydroxybenzene-1,3-dicarboxylic acid | | | | |
| (654) 2 uM | | | | |
| (2S)-N-(4-{[3-cyano-1-(2- | Doxorubicin | 0.07 | 0.046 | MDA-MB-468 |
| methylpropyl)-1H-indol-5- | | | | |
| yl]oxy}phenyl)pyrrolidine-2- | | | | |
| carboxamide 2 uM | | | | |
| N-(4-{[3-cyano-1-(2-methylpropyl)- | Paclitaxel | 0.01 | 0.0057 | MDA-MB-468 |
| 1H-indol-5- | | | | |
| yl]oxy}phenyl)pyrrolidine-2- | | | | |
| carboxamide 2 uM | | | | |
| AZ67 | Niraparib | 31 | 10 | BT-549 |
| AZ67 | Niraparib | 36 | 18.3 | Hs-578T |
| 25 uM 3-[3-methyl-5-(propan-2-yl)-1- | Niraparib | 36 | 27.9 | Hs-578T |
| benzothiophene-2- | | | | |
| sulfonamido]benzoic acid (QPD- | | | | |
| 14071) | | | | |
| 25 uM Compound 1 | Niraparib | 14.5 | 12.5 | MCF-7 |
| 25 uM 3-[3-methyl-5-(propan-2-yl)-1- | Niraparib | 14.6 | 13.2 | MDA-MB-231 |
| benzothiophene-2- | | | | |
| sulfonamido]benzoic acid (QPD- | | | | |
| 14071) | | | | |
| 25 uM 3-[3-methyl-5-(propan-2-yl)-1- | Olaparib | 10.5 | 9.66 | MDA-MB-468 |
| benzothiophene-2- | | | | |
| sulfonamido]benzoic acid (QPD- | | | | |
| 14071) | | | | |
| 25 uM Compound 1 | Niraparib | 21 | 13.8 | T-47D |
| 10 uM AZ67 | Olaparib | 38.6 | 30.6 | T-47D |
| 25 uM 3-[3-methyl-5-(propan-2-yl)-1- | Olaparib | 38.6 | 18.6 | T-47D |
| benzothiophene-2- | | | | |
| sulfonamido]benzoic acid (QPD- | | | | |
| 14071) | | | | |

The results are presented on table EXXX-1

As a prophetic example the comparable results could be achieved by the following protocol-cells are collected and plated into 96-well white tissue culture plates (Corning, Cat. No. 3610) up to the final volume 100 μl. All cells can be incubated in the plates overnight at 37° C., 5% 002 incubator with different medium.

### Example XXXI (Prophetic)

The Radial Glial (RG) cells and brain tumor-initiating cells derived cell lines (TCL) can be plated at a density of 300 cells/ml on 24 well plates in ENStem-A neural expansion medium with FGF2 (20 ng/ml), L-glutamine (2 mM) and PenStrep 1× and grown for 14 days at 37° C., 5% CO2 in a humidified atmosphere. The neurospheres can be collected and plated on laminin-coated tissue culture plates using the same media for 24-48 hours at 37° C., 5% CO2 in a humidified atmosphere. Cells can be detached using Acutase (Millipore) and neurospheres can be formed again as described above to produce self-renewal cell culture. Neurospheres can be then treated with any one of the compounds of this invention, e.g. but not limited to by Compound 1 or AZ67 at 1, 10, 30 μM each. Compounds of this invention demonstrate inhibition of cell proliferation.

### Example XXXII: In Vitro Model of Batten Disease

The objective of the study was to evaluate the efficacy of AZ67 in an in vitro model of Batten disease, using neurons from Cln7 knock out (KO) mice. To do so, glycolytic flux and apoptosis were measured in neurons from wild type (WT) and KO animals and the effect of PFKFB3 inhibitor on these parameters was evaluated.

Primary cultures of C57BL/6J WT and Cln7Δex2 mice cortical neurons were prepared from fetal animals of 14.5 days of gestation, seeded at 1.8·10⁵ cells/cm2 in plastic plates coated with poly-D-lysine (10 mg/ml) and incubated in Neurobasal (Life Technologies) supplemented with 2 mM glutamine, 5 mM of glucose, 0.25 mM pyruvate and 2% B27 supplement (Life Technologies). Cells were incubated at 37° C. in a humidified 5% CO2-containing atmosphere. At 72 hours after plating, medium was replaced using Neurobasal (Life Technologies) supplemented with 2 mM glutamine, 5 mM glucose, 0.25 mM pyruvate and 2% B27 supplement (Life Technologies) minus antioxidants (MAO; i.e., lacking vitamin E, vitamin E acetate, superoxide dismutase, catalase, and glutathione). Six days after the plating medium was replaced again. Cells were used at day 9.

Glycolytic flux was measured in 8 cm2 flasks of adherent cells at 60-70% confluence containing a central microcentrifuge tube with 1 ml H2O for 3H2O equilibration. Cells were incubated with 10 nM AZ67 or vehicle for 24 hours in KRPG containing 5.5 mM D-glucose at 37° C. in the air-thermostatized chamber of an orbital shaker (Forma Benchtop Orbital Shaker, Model 420, Thermo Fischer). To ensure adequate oxygen supply for oxidative metabolism throughout the incubation period, flasks were filled with oxygen before being sealed. Glycolytic flux was measured by assaying the rate of 3H2O production from [3-3H]glucose by incubating cells with 5μ Ci D-[3-3H]glucose in KRPG buffer per flask for 120 min, as previously described [Vicente-Gutierrez, C. et al. Astrocytic mitochondrial ROS modulate brain metabolism and mouse behavior. Nat Metabol 1, 201-211 (2019)]. Incubations were then terminated with 0.2 ml 20% perchloric acid, and the cells were further incubated for 96 h with a microcentrifuge tube containing H2O, suspended above the cells to allow 3H2O equilibration. The 3H2O was then measured by liquid scintillation counting (Tri-Carb 4810 TR, PerkinElmer). Under these experimental conditions, 28% of the produced 3H2O was recovered and used for the calculations as previously established [Herrero-Mendez, A. et al. The bioenergetic and antioxidant status of neurons is controlled by continuous degradation of a key glycolytic enzyme by APC/C-Cdh1. Nat Cell Biol 11, 747-752 (2009); Vicente-Gutierrez, C. et al. Astrocytic mitochondrial ROS modulate brain metabolism and mouse behavior. Nat Metabol 1, 201-211 (2019)]. As shown in FIG. 12 rate of glycolysis is increased in neurons from Cln7 KO mice compared to neurons from WT, AZ67 was able to reduce the excessive glycolysis to normal levels.

FIG. 12. Rate of glycolysis in neurons WT and Cln7 KO mice.

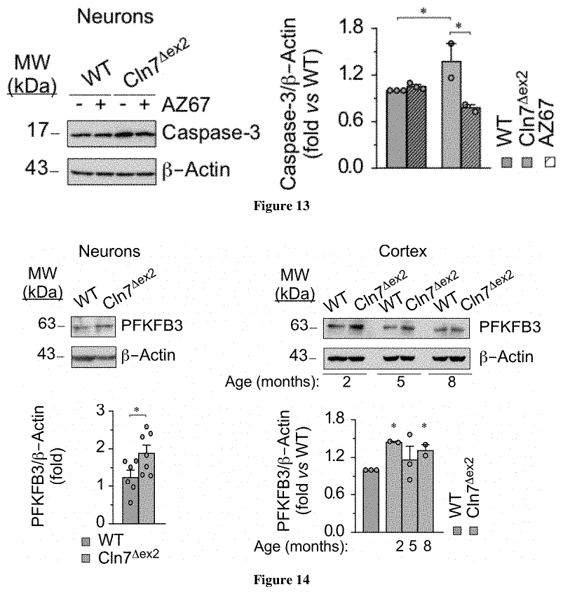

Neurons were incubated with 10 nM or vehicle for 24 hours and apoptosis was evaluated by measuring the level of active Caspase-3 form using Western blot. Neurons were lysed in RIPA buffer (2% sodium dodecylsulphate, 2 mM EDTA, 2 mM EGTA and 50 mM Tris pH 7.5), supplemented with protease and phosphatase inhibitor cocktail (100 μM phenylmethylsulfonyl fluoride, 50 μg/ml antipapain, 50 μg/ml pepstatin, 50 μg/ml amastatin, 50 μg/ml leupeptin, 50 μg/ml bestatin, 1 mM o-vanadate, 50 mM NaF, and 50 μg/ml soybean trypsin inhibitor) and boiled for 5 min. Extracts were centrifuged at 13,000×g for 5 min at 4° C., and aliquots of lysates (50 μg protein, unless otherwise stated) were subjected to sodium dodecyl sulfate-polyacrylamide (SDS-PAGE) electrophoresis on an 8, 10 or 12% acrylamide gel (MiniProtean, Bio-Rad) including PageRuler Plus Prestained Protein Ladder (Thermo). The resolved proteins were transferred electrophoretically to nitrocellulose membranes (Hybond-ECL, Amersham Bioscience Europe GmbH, Barcelona, Spain). Membranes were blocked with 5% (w/v) low-fat milk in 20 mM Tris, 500 mM NaCl, and 0.1% (w/v) Tween 20, pH 7.5, for 1 h. After blocking, membranes were immunoblotted with primary antibodies at dilutions ranging from 1:500 to 1:40,000 overnight at 4° C. After incubation with the secondary antibodies (all at 1:10,000 dilution), membranes were immediately incubated with the enhanced chemiluminescence kit WesternBright ECL (Advansta, Menlo Park, Calif., USA) for 2 min or SuperSignal West Femto Maximum Sensitivity Substrate (Thermo Scientific, Offenbach, Germany) for 5 min, before exposure to Fuji Medical X-Ray film (Fujifilm), and the autoradiograms scanned. The level of the active form of Caspase-3 was increased in neurons from KO mice compared to WT, demonstrating an elevated level of apoptosis (FIG. 13). Apoptosis level was significantly reduced in KO neurons after incubation with AZ67

FIG. 13. Evaluation of apoptosis level in neurons by measuring active Caspase-3 using Western blot

### Example XXXIII: In Vivo Model of Batten Disease (Prophetic)

Cln7 KO mice can be used as an in vivo model of Batten disease. In FIG. 14 we demonstrate that the level of PFKFB3 protein is increased in Cln7 KO mice compared to WT animals. PFKFB3 protein expression was measured by Western blot as described in example XXXII. Therefore, treatment of Cln7 mice with PFKFB3 inhibitors might alleviate symptoms of Batten disease and slow down the course of the disease

FIG. 14. Expression of PFKFB3 protein in Cln7 KO and WT mice measured by Western blot.

### Example XXXIV: In Vitro and In Vivo Model of Batten Disease (Prophetic)

Compound 1 or any other PFKFB3 inhibitor mentioned in this document can be tested in in vitro and in vivo model of Batten disease the same way as described in Examples XXXII and Example XXXIII above correspondently. One other of many possible methods to test such compounds in-vivo is as shown in Example VI: Chronic administration (Prophetic) above.

TABLE 5
Some of the known PFKFB3 inhibitors with the references on more details on it.
ID # IUPAC # Document # Title # Reference
AZ60 # (2S)-N-{4-[(3-cyano-1-ethyl-1H-indol-5-yl)oxy]phenyl}-5-oxopyrrolidine-2-carboxamide # PUBLICATION #
Structure-Based Design of Potent and Selective Inhibitors of the Metabolic Kinase PFKFB3. #
10.1021/acs.jmedchem.5b00352
AZ69 # 2-amino-N-(4-{[3-(1-methyl-1H-pyrazol-4-yl)-1H-indol-5-yl]oxy}phenyl)acetamide # PUBLICATION # Structure-
Based Design of Potent and Selective Inhibitors of the Metabolic Kinase PFKFB3. # 10.1021/acs.jmedchem.5b00352
CHEMBL234338 # 4,6,7,8-tetrahydroxy-2-(4-hydroxyphenyl)-5H-chromen-5-one # PUBLICATION # Targeting the
Warburg Effect in cancer; relationships for 2-arylpyridazinones as inhibitors of the key glycolytic enzyme 6-
phosphofructo-2-kinase/2,6-bisphosphatase 3 (PFKFB3). # 10.1016/j.bmc.2013.12.041
CHEMBL242739 # 7,8-dihydroxy-3-(4-hydroxyphenyl)-4H-chromen-4-one # PUBLICATION # Targeting the Warburg
Effect in cancer; relationships for 2-arylpyridazinones as inhibitors of the key glycolytic enzyme 6-phosphofructo-2-
kinase/2,6-bisphosphatase 3 (PFKFB3). # 10.1016/j.bmc.2013.12.041
CHEMBL3105825 # ethyl 1-(5-{2-oxa-6-azaspiro[3.3]heptan-6-yl}-6-oxo-1-phenyl-1,6-dihydropyridazin-4-yl)-
1H-1,2,3-triazole-4-carboxylate # PUBLICATION # Targeting the Warburg Effect in cancer; relationships for 2-arylpyridazinones as
inhibitors of the key glycolytic enzyme 6-phosphofructo-2-kinase/2,6-bisphosphatase 3 (PFKFB3). #
10.1016/j.bmc.2013.12.041
CHEMBL3105826 # 4-[4-(4-acetyl-5-methyl-1H-1,2,3-triazol-1-yl)-5-bromo-6-oxo-1,6-dihydropyridazin-1-yl]benzonitrile #
PUBLICATION # Targeting the Warburg Effect in cancer; relationships for 2-arylpyridazinones as inhibitors of the key
glycolytic enzyme 6-phosphofructo-2-kinase/2,6-bisphosphatase 3 (PFKFB3). # 10.1016/j.bmc.2013.12.041
CHEMBL3105827 # 5-(4-acetyl-5-methyl-1H-1,2,3-triazol-1-yl)-4-bromo-2-[4-(trifluoromethoxy)phenyl]-2,3-
dihydropyridazin-3-one # PUBLICATION # Targeting the Warburg Effect in cancer; relationships for 2-arylpyridazinones
as inhibitors of the key glycolytic enzyme 6-phosphofructo-2-kinase/2,6-bisphosphatase 3 (PFKFB3). #
10.1016/j.bmc.2013.12.041
CHEMBL3105828 # 5-(4-acetyl-5-methyl-1H-1,2,3-triazol-1-yl)-4-bromo-2-(4-chlorophenyl)-2,3-dihydropyridazin-3-one #
PUBLICATION # Targeting the Warburg Effect in cancer; relationships for 2-arylpyridazinones as inhibitors of the key
glycolytic enzyme 6-phosphofructo-2-kinase/2,6-bisphosphatase 3 (PFKFB3). # 10.1016/j.bmc.2013.12.041
CHEMBL3105829 # 5-(4-acetyl-5-methyl-1H-1,2,3-triazol-1-yl)-4-bromo-2-(pyrimidin-5-yl)-2,3-dihydropyridazin-3-one #
PUBLICATION # Targeting the Warburg Effect in cancer; relationships for 2-arylpyridazinones as inhibitors of the key
glycolytic enzyme 6-phosphofructo-2-kinase/2,6-bisphosphatase 3 (PFKFB3). # 10.1016/j.bmc.2013.12.041
CHEMBL3105830 # 5-(4-acetyl-5-methyl-1H-1,2,3-triazol-1-yl)-2-benzyl-4-bromo-2,3-dihydropyridazin-3-one #
PUBLICATION # Targeting the Warburg Effect in cancer; relationships for 2-arylpyridazinones as inhibitors of the key
glycolytic enzyme 6-phosphofructo-2-kinase/2,6-bisphosphatase 3 (PFKFB3). # 10.1016/j.bmc.2013.12.041
CHEMBL3105832 # 5-(4-acetyl-5-methyl-1H-1,2,3-triazol-1-yl)-4-bromo-2-[(4-iodophenyl)methyl]-2,3-dihydropyridazin-3-
one # PUBLICATION # Targeting the Warburg Effect in cancer; relationships for 2-arylpyridazinones as inhibitors of the
key glycolytic enzyme 6-phosphofructo-2-kinase/2,6-bisphosphatase 3 (PFKFB3). # 10.1016/j.bmc.2013.12.041
CHEMBL3105833 # 5-(4-acetyl-5-methyl-1H-1,2,3-triazol-1-yl)-4-bromo-2-(2-phenylethyl)-2,3-dihydropyridazin-3-one #
PUBLICATION # Targeting the Warburg Effect in cancer; relationships for 2-arylpyridazinones as inhibitors of the key
glycolytic enzyme 6-phosphofructo-2-kinase/2,6-bisphosphatase 3 (PFKFB3). # 10.1016/j.bmc.2013.12.041
CHEMBL3105834 # 5-(4-acetyl-5-methyl-1H-1,2,3-triazol-1-yl)-4-bromo-2-(3-phenylpropyl)-2,3-dihydropyridazin-3-one #
PUBLICATION # Targeting the Warburg Effect in cancer; relationships for 2-arylpyridazinones as inhibitors of the key
glycolytic enzyme 6-phosphofructo-2-kinase/2,6-bisphosphatase 3 (PFKFB3). # 10.1016/j.bmc.2013.12.041
CHEMBL3105838 # 2-hydroxy-4-(naphthalene-1-sulfonamido)benzoic acid # PUBLICATION # Targeting the Warburg
Effect in cancer; relationships for 2-arylpyridazinones as inhibitors of the key glycolytic enzyme 6-phosphofructo-2-
kinase/2,6-bisphosphatase 3 (PFKFB3). # 10.1016/j.bmc.2013.12.041
CHEMBL3105839 # 5-(4-acetyl-5-methyl-1H-1,2,3-triazol-1-yl)-4-bromo-2-phenyl-2,3-dihydropyridazin-3-one #
PUBLICATION # Targeting the Warburg Effect in cancer; relationships for 2-arylpyridazinones as inhibitors of the key
glycolytic enzyme 6-phosphofructo-2-kinase/2,6-bisphosphatase 3 (PFKFB3). # 10.1016/j.bmc.2013.12.041
CHEMBL3105844 # 5-(4-acetyl-5-methyl-1H-1,2,3-triazol-1-yl)-4-chloro-2-phenyl-2,3-dihydropyridazin-3-one #
PUBLICATION # Targeting the Warburg Effect in cancer; relationships for 2-arylpyridazinones as inhibitors of the key
glycolytic enzyme 6-phosphofructo-2-kinase/2,6-bisphosphatase 3 (PFKFB3). # 10.1016/j.bmc.2013.12.041
CHEMBL3105845 # 5-(4-acetyl-5-methyl-1H-1,2,3-triazol-1-yl)-4-iodo-2-phenyl-2,3-dihydropyridazin-3-one #
PUBLICATION # Targeting the Warburg Effect in cancer; relationships for 2-arylpyridazinones as inhibitors of the key
glycolytic enzyme 6-phosphofructo-2-kinase/2,6-bisphosphatase 3 (PFKFB3). # 10.1016/j.bmc.2013.12.041
CHEMBL3421731 # (2S)-N-(4-{[1-methyl-3-(1-methyl-1H-pyrazol-4-yl)-1H-indol-5-yl]oxy}phenyl)pyrrolidine-2-
carboxamide # PUBLICATION # Structure-Based Design of Potent and Selective Inhibitors of the Metabolic Kinase
PFKFB3. # 10.1021/acs.jmedchem.5b00352
CHEMBL3421732 # (2S)-N-(4-{[3-cyano-1-(2-methylpropyl)-1H-indol-5-yl]amino}phenyl)pyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3421733 # (2S)-N-(4-{[3-cyano-1-(2-methylpropyl)-1H-indol-5-yl](methyl)amino}phenyl)pyrrolidine-2-
carboxamide # PUBLICATION # Structure-Based Design of Potent and Selective Inhibitors of the Metabolic Kinase
PFKFB3. # 10.1021/acs.jmedchem.5b00352
CHEMBL3421734 # (2S)-N-(4-{[3-cyano-1-(2-methylpropyl)-1H-indol-5-yl]sulfanyl}phenyl)pyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3421735 # (2S)-N-(4-([3-cyano-1-(2-methylpropyl)-1H-indol-5-yl]sulfonyl}phenyl)pyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3421736 # (2S)-N-(4-([3-cyano-1-(2-methylpropyl)-1H-indol-5-yl]methyl}phenyl)pyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3422651 # (2S)-N-(4-{[3-cyano-1-(2-methylpropyl)-1H-indol-5-yl]oxy}phenyl)pyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3422652 # 2-amino-N-{4-[(2-amino-3-cyano-1-ethyl-1H-indol-5-yl)oxy]phenyl}acetamide # PUBLICATION #
Structure-Based Design of Potent and Selective Inhibitors of the Metabolic Kinase PFKFB3. #
10.1021/acs.jmedchem.5b00352
CHEMBL3422653 # 2-amino-N-{4-[(2-amino-3-cyano-1-methyl-1H-indol-5-yl)oxy]phenyl}acetamide # PUBLICATION #
Structure-Based Design of Potent and Selective Inhibitors of the Metabolic Kinase PFKFB3. #
10.1021/acs.jmedchem.5b00352
CHEMBL3422654 # (2S)-2-amino-N-{4-[(2-amino-3-cyano-1H-indol-5-yl)oxy]phenyl}-3-hydroxypropanamide #
PUBLICATION # Structure-Based Design of Potent and Selective Inhibitors of the Metabolic Kinase PFKFB3. #
10.1021/acs.jmedchem.5b00352
CHEMBL3422656 # 2-amino-N-{4-[(2-amino-3-cyano-1H-indol-5-yl)oxy]phenyl}acetamide # PUBLICATION # Structure-
Based Design of Potent and Selective Inhibitors of the Metabolic Kinase PFKFB3. # 10.1021/acs.jmedchem.5b00352
CHEMBL3422657 # 2-amino-N-(4-{[2-amino-3-cyano-1-(2-methylpropyl)-1H-indol-5-yl]oxy}phenyl)acetamide #
PUBLICATION # Structure-Based Design of Potent and Selective Inhibitors of the Metabolic Kinase PFKFB3. #
10.1021/acs.jmedchem.5b00352
CHEMBL3422658 # 2-amino-N-{4-[(2-amino-1-benzyl-3-cyano-1H-indol-5-yl)oxy]phenyl}acetamide # PUBLICATION #
Structure-Based Design of Potent and Selective Inhibitors of the Metabolic Kinase PFKFB3. #
10.1021/acs.jmedchem.5b00352
CHEMBL3422659 # 2-{2-amino-5-[4-(2-aminoacetamido)phenoxy]-3-cyano-1H-indol-1-yl}-N,N-dimethylacetamide #
PUBLICATION # Structure-Based Design of Potent and Selective Inhibitors of the Metabolic Kinase PFKFB3. #
10.1021/acs.jmedchem.5b00352
CHEMBL3422660 # 2-amino-N-(4-[(3-cyano-1H-indol-5-yl)oxy]phenyl}acetamide # PUBLICATION # Structure-Based
Design of Potent and Selective Inhibitors of the Metabolic Kinase PFKFB3. # 10.1021/acs.jmedchem.5b00352
CHEMBL3422661 # 2-amino-N-{4-[(3-cyano-1-ethyl-1H-indol-5-yl)oxy]phenyl}acetamide # PUBLICATION # Structure-
Based Design of Potent and Selective Inhibitors of the Metabolic Kinase PFKFB3. # 10.1021/acs.jmedchem.5b00352
CHEMBL3422662 # N-{4-[(3-cyano-1-ethyl-1H-indol-5-yl)oxy]phenyl}-2-(methylamino)acetamide # PUBLICATION #
Structure-Based Design of Potent and Selective Inhibitors of the Metabolic Kinase PFKFB3. #
10.1021/acs.jmedchem.5b00352
CHEMBL3422663 # N-{4-[(3-cyano-1-ethyl-1H-indol-5-yl)oxy]phenyl}-2-(dimethylamino)acetamide # PUBLICATION #
Structure-Based Design of Potent and Selective Inhibitors of the Metabolic Kinase PFKFB3. #
10.1021/acs.jmedchem.5b00352
CHEMBL3422664 # (2S)-N-{4-[(3-cyano-1-ethyl-1H-indol-5-yl)oxy]phenyl}pyrrolidine-2-carboxamide # PUBLICATION #
Structure-Based Design of Potent and Selective Inhibitors of the Metabolic Kinase PFKFB3. #
10.1021/acs.jmedchem.5b00352
CHEMBL3422665 # (2R)-N-{4-[(3-cyano-1-ethyl-1H-indol-5-yl)oxy]phenyl}pyrrolidine-2-carboxamide # PUBLICATION #
Structure-Based Design of Potent and Selective Inhibitors of the Metabolic Kinase PFKFB3. #
10.1021/acs.jmedchem.5b00352
CHEMBL3422666 # (2S)-N-{4-[(3-cyano-1-ethyl-1H-indol-5-yl)oxy]phenyl}-N-methylpyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3422667 # (2S)-N-{4-[(3-cyano-1-ethyl-1H-indol-5-yl)oxy]phenyl}azetidine-2-carboxamide # PUBLICATION #
Structure-Based Design of Potent and Selective Inhibitors of the Metabolic Kinase PFKFB3. #
10.1021/acs.jmedchem.5b00352
CHEMBL3422668 # (2S)-N-{4-[(3-cyano-1-ethyl-1H-indol-5-yl)oxy]phenyl}piperidine-2-carboxamide # PUBLICATION #
Structure-Based Design of Potent and Selective Inhibitors of the Metabolic Kinase PFKFB3. #
10.1021/acs.jmedchem.5b00352
CHEMBL3422669 # (2S)-N-{4-[(3-cyano-1-ethyl-1H-indol-5-yl)oxy]phenyl}-N-methyl-5-oxopyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3422670 # (2S)-N-[4-((3-cyano-1-[(methylcarbamoyl)methyl]-1H-indol-5-yl}oxy)phenyl]pyrrolidine-2-
carboxamide # PUBLICATION # Structure-Based Design of Potent and Selective Inhibitors of the Metabolic Kinase
PFKFB3. # 10.1021/acs.jmedchem.5b00352
CHEMBL3422671 # (2S)-N-[4-({3-cyano-1-[2-(dimethylamino)ethyl]-1H-indol-5-yl}oxy)phenyl]pyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3422672 # (2S)-N-[4-({3-cyano-1-[(oxan-4-yl)methyl]-1H-indol-5-yl}oxy)phenyl]pyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3422673 # (2S)-N-{4-[(3-cyano-1-phenyl-1H-indol-5-yl)oxy]phenyl}pyrrolidine-2-carboxamide # PUBLICATION #
Structure-Based Design of Potent and Selective Inhibitors of the Metabolic Kinase PFKFB3. #
10.1021/acs.jmedchem.5b00352
CHEMBL3422674 # (2S)-N-(4-{[3-cyano-1-(2-methyl-1-oxo-2,3-dihydro-1H-isoindol-5-yl)-1H-indol-5-
yl]oxy}phenyl)pyrrolidine-2-carboxamide # PUBLICATION # Structure-Based Design of Potent and Selective Inhibitors of
the Metabolic Kinase PFKFB3. # 10.1021/acs.jmedchem.5b00352
CHEMBL3422675 # (2S)-N-{4-[(1-benzyl-3-cyano-1H-indol-5-yl)oxy]phenyl}pyrrolidine-2-carboxamide # PUBLICATION #
Structure-Based Design of Potent and Selective Inhibitors of the Metabolic Kinase PFKFB3. #
10.1021/acs.jmedchem.5b00352
CHEMBL3422676 (AZ67) # (2S)-N-[4-({3-cyano-1-[(3,5-dimethyl-1,2-oxazol-4-yl)methyl]-1H-indol-5-
yl}oxy)phenyl]pyrrolidine-2-carboxamide # PUBLICATION # Structure-Based Design of Potent and Selective Inhibitors of
the Metabolic Kinase PFKFB3. # 10.1021/acs.jmedchem.5b00352
CHEMBL3422677 # (2S)-N-(4-{[3-cyano-1-(2-methylpropyl)-1H-indazol-5-yl]oxy}phenyl)pyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3422678 # (2S)-N-[4-({3-cyano-1-[(3,5-dimethyl-1,2-oxazol-4-yl)methyl]-1H-indazol-5-yl}oxy)phenyl]pyrrolidine-
2-carboxamide # PUBLICATION # Structure-Based Design of Potent and Selective Inhibitors of the Metabolic Kinase
PFKFB3. # 10.1021/acs.jmedchem.5b00352
CHEMBL3422679 # 2-amino-N-(4-{[3-(1-methyl-1H-pyrazol-4-yl)-1H-indazol-5-yl]oxy}phenyl)acetamide # PUBLICATION
# Structure-Based Design of Potent and Selective Inhibitors of the Metabolic Kinase PFKFB3. #
10.1021/acs.jmedchem.5b00352
CHEMBL3422680 # (2S)-N-(4-{[3-(1-methyl-1H-pyrazol-4-yl)-1H-indol-5-yl]oxy}phenyl)pyrrolidine-2-carboxamide #
PUBLICATION # Structure-Based Design of Potent and Selective Inhibitors of the Metabolic Kinase PFKFB3. #
10.1021/acs.jmedchem.5b00352
CHEMBL3727388 # N-{1-[3-(2-aminopyrimidin-5-yl)-4-methylphenyl]ethyl}-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3727389 # 6-(1,3-benzothiazol-5-yl)-2-methyl-N-{1-[3-(1-methyl-1H-pyrazol-4-yl)phenyl]ethyl}pyrimidin-4-amine
# PATENT # Inhibitors of inducible form of 6-phosphofructose-2-kinase # EP-2702043-A1
CHEMBL3727394 # [1-(5-{3-[(1S)-1-{[2-methyl-6-(3-methyl-1-benzofuran-5-yl)pyrimidin-4-
yl]amino}ethyl]phenyl}pyrimidin-2-yl)piperidin-4-yl]methanol # PATENT # Inhibitors of inducible form of 6-
phosphofructose-2-kinase # EP-2702043-A1
CHEMBL3727439 # 2-methyl-N-{1-[3-(1-methyl-1H-pyrazol-4-yl)phenyl]ethyl}-6-phenylpyrimidin-4-amine # PATENT #
Inhibitors of inducible form of 6-phosphofructose-2-kinase # EP-2702043-A1
CHEMBL3727454 # 6-(1H-indol-6-yl)-2-methyl-N-[(1R)-1,2,3,4-tetrahydronaphthalen-1-yl]pyrimidin-4-amine # PATENT #
Inhibitors of inducible form of 6-phosphofructose-2-kinase # EP-2702043-A1
CHEMBL3727468 # 2-[3-(1-{[6-(1,3-benzothiazol-6-yl)-2-methylpyrimidin-4-yl]amino}ethyl)phenoxy]acetonitrile #
PATENT # Inhibitors of inducible form of 6-phosphofructose-2-kinase # EP-2702043-A1
CHEMBL3727474 # 6-(1,3-benzothiazol-6-yl)-2-methyl-N-(1-{3-[(1,2-oxazol-3-yl)methoxy]phenyl}ethyl)pyrimidin-4-amine
# PATENT # Inhibitors of inducible form of 6-phosphofructose-2-kinase # EP-2702043-A1
CHEMBL3727489 # 6-(2,3-dihydro-1-benzofuran-5-yl)-N-[(1S)-1-(3-methoxyphenyl)ethyl]-2-methylpyrimidin-4-amine #
PATENT # Inhibitors of inducible form of 6-phosphofructose-2-kinase # EP-2702043-A1
CHEMBL3727496 # 2-methyl-6-(3-methyl-1-benzofuran-5-yl)-N-[(1S)-1-[5-(1-methyl-1H-pyrazol-4-yl)pyridin-3-
yl]ethyl]pyrimidin-4-amine # PATENT # Inhibitors of inducible form of 6-phosphofructose-2-kinase # EP-2702043-A1
CHEMBL3727501 # 2-methyl-N-(1-{3-[(5-methyl-1,2,4-oxadiazol-3-yl)methoxy]phenyl}ethyl)-6-(3-methyl-1-benzofuran-5-
yl)pyrimidin-4-amine # PATENT # Inhibitors of inducible form of 6-phosphofructose-2-kinase # EP-2702043-A1
CHEMBL3727504 # 6-(2-fluoro-3-methoxyphenyl)-2-methyl-N-{1-[3-(1-methyl-1H-pyrazol-4-yl)phenyl]ethyl}pyrimidin-4-
amine # PATENT # Inhibitors of inducible form of 6-phosphofructose-2-kinase # EP-2702043-A1
CHEMBL3727507 # N-{1-[5-(2-aminopyrimidin-5-yl)pyridin-3-yl]ethyl}-2-methyl-6-(3-methyl-1-benzofuran-5-yl)pyrimidin-
4-amine # PATENT # Inhibitors of inducible form of 6-phosphofructose-2-kinase # EP-2702043-A1
CHEMBL3727512 # N-[(1S)-1-[3-(5-chloropyridin-3-yl)phenyl]ethyl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3727523 # N-[(1S)-1-[3-(4-chloropyridin-3-yl)phenyl]ethyl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3727524 # 2-[3-(1-{[6-(1,3-benzothiazol-6-yl)-2-methylpyrimidin-4-yl]amino}ethyl)phenoxy]-N-ethyl-N-methylacetamide #
PATENT # Inhibitors of inducible form of 6-phosphofructose-2-kinase # EP-2702043-A1
CHEMBL3727541 # 6-{4-chloro-3-[(prop-2-yn-1-yl)amino]phenyl}-2-methyl-N-[(1R)-1,2,3,4-tetrahydronaphthalen-1-
yl]pyrimidin-4-amine # PATENT # Inhibitors of inducible form of 6-phosphofructose-2-kinase # EP-2702043-A1
CHEMBL3727563 # N-[(3-methoxyphenyl)methyl]-2-methyl-6-(3-methyl-1-benzofuran-5-yl)pyrimidin-4-amine # PATENT
# Inhibitors of inducible form of 6-phosphofructose-2-kinase # EP-2702043-A1
CHEMBL3727566 # 2-methyl-6-(2-phenylethenyl)-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3727572 # 4-(2-methyl-6-{[(1R)-1,2,3,4-tetrahydronaphthalen-1-yl]amino}pyrimidin-4-yl)benzamide # PATENT #
Inhibitors of inducible form of 6-phosphofructose-2-kinase # EP-2702043-A1
CHEMBL3727575 # 3-({[6-(1,3-benzothiazol-6-yl)-2-methylpyrimidin-4-yl]amino}methyl)-4-fluorophenol # PATENT #
Inhibitors of inducible form of 6-phosphofructose-2-kinase # EP-2702043-A1
CHEMBL3727576 # 2-(5-{3-[(1S)-1-{[2-methyl-6-(3-methyl-1-benzofuran-5-yl)pyrimidin-4-yl]amino}ethyl]phenyl}pyridin-3-
yl)propan-2-ol # PATENT # Inhibitors of inducible form of 6-phosphofructose-2-kinase # EP-2702043-A1
CHEMBL3727580 # 6-(3-ethyl-1-benzofuran-5-yl)-N-[(1S)-1-(3-methoxyphenyl)ethyl]-2-methylpyrimidin-4-amine #
PATENT # Inhibitors of inducible form of 6-phosphofructose-2-kinase # EP-2702043-A1
CHEMBL3727607 # 6-(1,3-benzothiazol-6-yl)-N-[(2-chloro-6-fluorophenyl)methyl]-2-methylpyrimidin-4-amine # PATENT
# Inhibitors of inducible form of 6-phosphofructose-2-kinase # EP-2702043-A1
CHEMBL3727614 # 2-methyl-6-(3-methyl-1-benzofuran-5-yl)-N-[(1S)-1-{3-[2-(morpholin-4-yl)pyrimidin-5-
yl]phenyl}ethyl]pyrimidin-4-amine # PATENT # Inhibitors of inducible form of 6-phosphofructose-2-kinase # EP-2702043-
A1
CHEMBL3727615 # N1-[6-(1,3-benzothiazol-6-yl)-2-methylpyrimidin-4-yl]-1,2,3,4-tetrahydronaphthalene-1,6-diamine #
PATENT # Inhibitors of inducible form of 6-phosphofructose-2-kinase # EP-2702043-A1
CHEMBL3727628 # 2-methyl-6-(3-methyl-1-benzofuran-5-yl)-N-[(1S)-1-[3-(1H-1,2,3,4-tetrazol-1-yl)phenyl]ethyl]pyrimidin-
4-amine # PATENT # Inhibitors of inducible form of 6-phosphofructose-2-kinase # EP-2702043-A1
CHEMBL3727634 # 6-(1,3-benzothiazol-6-yl)-N-[(1S)-1-[3-(5-chloropyridin-3-yl)phenyl]ethyl]-2-methylpyrimidin-4-amine #
PATENT # Inhibitors of inducible form of 6-phosphofructose-2-kinase # EP-2702043-A1
CHEMBL3727636 # 2-methyl-6-(3-methyl-1-benzofuran-5-yl)-N-[(1S)-1-[3-(1H-pyrazol-3-yl)phenyl]ethyl]pyrimidin-4-
amine # PATENT # Inhibitors of inducible form of 6-phosphofructose-2-kinase # EP-2702043-A1
CHEMBL3727653 # N-[(1S)-1-{3-[6-(ethylamino)pyridin-3-yl]phenyl}ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3727669 # N-(cyanomethyl)-3-(1-{[2-methyl-6-(1-methyl-1H-indol-6-yl)pyrimidin-4-yl]amino}ethyl)benzene-1-
sulfonamide # PATENT # Inhibitors of inducible form of 6-phosphofructose-2-kinase # EP-2702043-A1
CHEMBL3727673 # N-[3-(1-{[6-(1,3-benzothiazol-6-yl)-2-methylpyrimidin-4-yl]amino}ethyl)phenyl]-1H-pyrazole-5-
carboxamide # PATENT # Inhibitors of inducible form of 6-phosphofructose-2-kinase # EP-2702043-A1
CHEMBL3727683 # 6-(1,3-benzothiazol-6-yl)-N-[(2-bromo-5-methoxyphenyl)methyl]-2-methylpyrimidin-4-amine #
PATENT # Inhibitors of inducible form of 6-phosphofructose-2-kinase # EP-2702043-A1
CHEMBL3727688 # 3-[(5-{3-[(1S)-1-{[6-(7-fluoro-3-methyl-1-benzofuran-5-yl)-2-methylpyrimidin-4-
yl]amino}ethyl]phenyl}pyrimidin-2-yl)amino]propane-1,2-diol # PATENT # Inhibitors of inducible form of 6-
phosphofructose-2-kinase # EP-2702043-A1
CHEMBL3727699 # 6-(1,3-benzothiazol-6-yl)-2-methyl-N-(1-{3-[(piperidin-3-yl)methoxy]phenyl}ethyl)pyrimidin-4-amine #
PATENT # Inhibitors of inducible form of 6-phosphofructose-2-kinase # EP-2702043-A1
CHEMBL3727708 # methyl 2-({5-[3-(1-{[2-methyl-6-(3-methyl-1-benzofuran-5-yl)pyrimidin-4-
yl]amino}ethyl)phenyl]pyrimidin-2-yl}amino)acetate # PATENT # Inhibitors of inducible form of 6-phosphofructose-2-
kinase # EP-2702043-A1
CHEMBL3727718 # 2-methyl-6-(3-methyl-1-benzofuran-5-yl)-N-[(1S)-1-(3-{2-[(propan-2-yl)amino]pyrimidin-5-
yl}phenyl)ethyl]pyrimidin-4-amine # PATENT # Inhibitors of inducible form of 6-phosphofructose-2-kinase # EP-2702043-A1
CHEMBL3727753 # 2-[3-(1-{[6-(1,3-benzothiazol-6-yl)-2-methylpyrimidin-4-yl]amino}ethyl)phenoxy]-N-ethylacetamide #
PATENT # Inhibitors of inducible form of 6-phosphofructose-2-kinase # EP-2702043-A1
CHEMBL3727780 # 6-(3-amino-4-chlorophenyl)-2-methyl-N-{1-[3-(1-methyl-1H-pyrazol-4-yl)phenyl]ethyl}pyrimidin-4-
amine # PATENT # Inhibitors of inducible form of 6-phosphofructose-2-kinase # EP-2702043-A1
CHEMBL3727782 # N-[(1S)-1-[3-(5-aminopyridin-3-yl)phenyl]ethyl]-2-methyl-6-(3-methyl-1-benzothiophen-5-yl)pyrimidin-
4-amine # PATENT # Inhibitors of inducible form of 6-phosphofructose-2-kinase # EP-2702043-A1
CHEMBL3727784 # 2-methyl-6-{1-methyl-1H-pyrrolo[3,2-b]pyridin-6-yl}-N-[(1R)-1,2,3,4-tetrahydronaphthalen-1-
yl]pyrimidin-4-amine # PATENT # Inhibitors of inducible form of 6-phosphofructose-2-kinase # EP-2702043-A1
CHEMBL3727790 # 6-(3,4-dichlorophenyl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3727808 # N-[(1S)-1-(3-bromophenyl)ethyl]-6-(3-ethyl-1-benzofuran-5-yl)-2-methylpyrimidin-4-amine # PATENT
# Inhibitors of inducible form of 6-phosphofructose-2-kinase # EP-2702043-A1
CHEMBL3727811 # 6-(1,3-benzothiazol-6-yl)-N-[(1S)-1-{3-[2-(dimethylamino)pyrimidin-5-yl]phenyl}ethyl]-2-
methylpyrimidin-4-amine # PATENT # Inhibitors of inducible form of 6-phosphofructose-2-kinase # EP-2702043-A1
CHEMBL3727826 # 6-(1,3-benzothiazol-6-yl)-2-methyl-N-[(1S)-1-[3-(1H-pyrazol-4-yl)phenyl]ethyl]pyrimidin-4-amine #
PATENT # Inhibitors of inducible form of 6-phosphofructose-2-kinase # EP-2702043-A1
CHEMBL3727839 # 2-methyl-6-[4-methyl-3-(methylamino)phenyl]-N-[(1R)-1,2,3,4-tetrahydronaphthalen-1-yl]pyrimidin-4-
amine # PATENT # Inhibitors of inducible form of 6-phosphofructose-2-kinase # EP-2702043-A1
CHEMBL3727850 # N-(1-{3-[(1,3-dimethyl-1H-pyrazol-5-yl)methoxy]phenyl}ethyl)-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3727851 # 6-(1,3-benzothiazol-6-yl)-2-methyl-N-{1-[3-(1H-pyrrol-2-yl)phenyl]ethyl}pyrimidin-4-amine # PATENT
# Inhibitors of inducible form of 6-phosphofructose-2-kinase # EP-2702043-A1
CHEMBL3727863 # 3-(1-{[6-(2-chloro-3-methoxyphenyl)-2-methylpyrimidin-4-yl]amino}ethyl)benzene-1-sulfonamide #
PATENT # Inhibitors of inducible form of 6-phosphofructose-2-kinase # EP-2702043-A1
CHEMBL3727869 # 2-methyl-6-(3-methyl-1-benzofuran-5-yl)-N-[1-(3-nitrophenyl)ethyl]pyrimidin-4-amine # PATENT #
Inhibitors of inducible form of 6-phosphofructose-2-kinase # EP-2702043-A1
CHEMBL3727871 # 6-(1,3-benzothiazol-6-yl)-2-methyl-N-{1-[3-(prop-2-yn-1-yloxy)phenyl]ethyl}pyrimidin-4-amine #
PATENT # Inhibitors of inducible form of 6-phosphofructose-2-kinase # EP-2702043-A1
CHEMBL3727892 # 6-(1,3-benzothiazol-6-yl)-N-[1-(2-chloropyridin-4-yl)ethyl]-2-methylpyrimidin-4-amine # PATENT #
Inhibitors of inducible form of 6-phosphofructose-2-kinase # EP-2702043-A1
CHEMBL3727895 # 3-(1-{[6-(1,3-benzothiazol-6-yl)-2-methylpyrimidin-4-yl]amino}ethyl)-N-(cyanomethyl)benzene-1-
sulfonamide # PATENT # Inhibitors of inducible form of 6-phosphofructose-2-kinase # EP-2702043-A1
CHEMBL3727921 # 2-methyl-6-(3-methyl-1-benzofuran-5-yl)-N-[(1S)-1-[2-(1-methyl-1H-pyrazol-4-yl)pyridin-4-
yl]ethyl]pyrimidin-4-amine # PATENT # Inhibitors of inducible form of 6-phosphofructose-2-kinase # EP-2702043-A1
CHEMBL3727928 # 6-(1-benzofuran-5-yl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3727931 # 6-(4-fluorophenyl)-2-methyl-N-[(1R)-1,2,3,4-tetrahydronaphthalen-1-yl]pyrimidin-4-amine # PATENT
# Inhibitors of inducible form of 6-phosphofructose-2-kinase # EP-2702043-A1
CHEMBL3727932 # 3-{5-[2-(methylsulfanyl)pyrimidin-5-yl]thiophene-2-sulfonamido}benzoic acid # PATENT # New
compounds # WO-2012035171-A2
CHEMBL3727948 # 2-{[3-(1-{[6-(1,3-benzothiazol-6-yl)-2-methylpyrimidin-4-yl]amino}ethyl)phenoxy]methyl}-1,3-oxazole-
4-carboxylic acid # PATENT # Inhibitors of inducible form of 6-phosphofructose-2-kinase # EP-2702043-A1
CHEMBL3727966 # 6-(7-fluoro-3-methyl-1-benzofuran-5-yl)-N-[(1S)-1-[3-(5-fluoropyridin-3-yl)phenyl]ethyl]-2-
methylpyrimidin-4-amine # PATENT # Inhibitors of inducible form of 6-phosphofructose-2-kinase # EP-2702043-A1
CHEMBL3727978 # 2-[3-(1-{[6-(3-methoxyphenyl)-2-methylpyrimidin-4-yl]amino}ethyl)phenoxy]-N-methylacetamide #
PATENT # Inhibitors of inducible form of 6-phosphofructose-2-kinase # EP-2702043-A1
CHEMBL3727985 # 6-(1,3-benzothiazol-6-yl)-2-methyl-N-(1-{3-[(5-methyl-1,2,4-oxadiazol-3-
yl)methoxy]phenyl}ethyl)pyrimidin-4-amine # PATENT # Inhibitors of inducible form of 6-phosphofructose-2-kinase # EP-
2702043-A1
CHEMBL3727988 # 6-(1,3-benzothiazol-6-yl)-2-methyl-N-{1-[3-(2,2,2-trifluoroethoxy)phenyl]ethyl}pyrimidin-4-amine #
PATENT # Inhibitors of inducible form of 6-phosphofructose-2-kinase # EP-2702043-A1
CHEMBL3728020 # N-(cyanomethyl)-3-(1-{[6-(3-methoxyphenyl)-2-methylpyrimidin-4-yl]amino}ethyl)benzene-1-
sulfonamide # PATENT # Inhibitors of inducible form of 6-phosphofructose-2-kinase # EP-2702043-A1
CHEMBL3728028 # 6-(3-chlorophenyl)-2-methyl-N-[(1R)-1,2,3,4-tetrahydronaphthalen-1-yl]pyrimidin-4-amine # PATENT
# Inhibitors of inducible form of 6-phosphofructose-2-kinase # EP-2702043-A1
CHEMBL3728032 # 2-methyl-6-(3-methyl-1-benzofuran-5-yl)-N-[(1R)-1-[5-(1-methyl-1H-pyrazol-4-yl)pyridin-3-
yl]ethyl]pyrimidin-4-amine # PATENT # Inhibitors of inducible form of 6-phosphofructose-2-kinase # EP-2702043-A1
CHEMBL3728037 # 1-(5-{3-[(1S)-1-{[2-methyl-6-(3-methyl-1-benzofuran-5-yl)pyrimidin-4-yl]amino}ethyl]phenyl}pyrimidin-
2-yl)piperidin-4-ol # PATENT # Inhibitors of inducible form of 6-phosphofructose-2-kinase # EP-2702043-A1
CHEMBL3728066 # 2-[2-methyl-5-(2-methyl-6-{[(1R)-1,2,3,4-tetrahydronaphthalen-1-yl]amino}pyrimidin-4-
yl)phenoxy]acetonitrile # PATENT # Inhibitors of inducible form of 6-phosphofructose-2-kinase # EP-2702043-A1
CHEMBL3728070 # 2-{[2-chloro-5-(2-methyl-6-{[(1R)-1,2,3,4-tetrahydronaphthalen-1-yl]amino}pyrimidin-4-
yl)phenyl]methoxy}propanoic acid # PATENT # Inhibitors of inducible form of 6-phosphofructose-2-kinase # EP-2702043-A1
CHEMBL3728078 # N-[(1S)-1-[3-(2-aminopyrimidin-5-yl)phenyl]ethyl]-6-(3,4-difluorophenyl)-2-methylpyrimidin-4-amine #
PATENT # Inhibitors of inducible form of 6-phosphofructose-2-kinase # EP-2702043-A1
CHEMBL3728085 # 6-(1,3-benzothiazol-6-yl)-N-(1-{3-[2-(dimethylamino)ethoxy]phenyl}ethyl)-2-methylpyrimidin-4-amine
# PATENT # Inhibitors of inducible form of 6-phosphofructose-2-kinase # EP-2702043-A1
CHEMBL3728086 # 3-(1-{[2-methyl-6-(1-methyl-1H-indol-6-yl)pyrimidin-4-yl]amino}ethyl)phenol # PATENT # Inhibitors of
inducible form of 6-phosphofructose-2-kinase # EP-2702043-A1
CHEMBL3728106 # 1-(3-{[3-(1-{[6-(1,3-benzothiazol-6-yl)-2-methylpyrimidin-4-yl]amino}ethyl)phenoxy]methyl}piperidin-1-
yl)ethan-1-one # PATENT # Inhibitors of inducible form of 6-phosphofructose-2-kinase # EP-2702043-A1
CHEMBL3728117 # 2-fluoro-5-(1-{[2-methyl-6-(3-methyl-1-benzofuran-5-yl)pyrimidin-4-yl]amino}ethyl)phenol # PATENT
# Inhibitors of inducible form of 6-phosphofructose-2-kinase # EP-2702043-A1
CHEMBL3728138 # ethyl 4-[3-(1-{[2-methyl-6-(1-methyl-1H-indol-6-yl)pyrimidin-4-yl]amino}ethyl)phenoxy]butanoate #
PATENT # Inhibitors of inducible form of 6-phosphofructose-2-kinase # EP-2702043-A1
CHEMBL3728160 # [4-(2-methyl-6-{[(1R)-1,2,3,4-tetrahydronaphthalen-1-yl]amino}pyrimidin-4-yl)phenyl]methanol #
PATENT # Inhibitors of inducible form of 6-phosphofructose-2-kinase # EP-2702043-A1
CHEMBL3728161 # 2-[3-(1-{[6-(1,3-benzothiazol-6-yl)-2-methylpyrimidin-4-yl]amino}ethyl)phenoxy]-N-cyclohexylacetamide #
PATENT # Inhibitors of inducible form of 6-phosphofructose-2-kinase # EP-2702043-A1
CHEMBL3728162 # 6-(1,3-benzothiazol-6-yl)-2-methyl-N-[(1S)-1-[3-(5-methylpyridin-3-yl)phenyl]ethyl]pyrimidin-4-amine
# PATENT # Inhibitors of inducible form of 6-phosphofructose-2-kinase # EP-2702043-A1
CHEMBL3728163 # 2-methyl-6-(1-methyl-1H-indol-6-yl)-N-[(1S)-1-(4-methylphenyl)ethyl]pyrimidin-4-amine # PATENT #
Inhibitors of inducible form of 6-phosphofructose-2-kinase # EP-2702043-A1
CHEMBL3728169 # N-[(1S)-1-[3-(1-ethyl-1H-pyrazol-4-yl)phenyl]ethyl]-2-methyl-6-(3-methyl-1-benzofuran-5-yl)pyrimidin-
4-amine # PATENT # Inhibitors of inducible form of 6-phosphofructose-2-kinase # EP-2702043-A1
CHEMBL3728172 # N-[(1S)-1-[3-(6-fluoropyridin-3-yl)phenyl]ethyl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3728178 # 3-[(5-{3-[(1S)-1-{[6-(4-chloro-3-fluorophenyl)-2-methylpyrimidin-4-yl]amino}ethyl]phenyl}pyrimidin-2-
yl)amino]propane-1,2-diol # PATENT # Inhibitors of inducible form of 6-phosphofructose-2-kinase # EP-2702043-A1
CHEMBL3728185 # 6-(1H-indol-5-yl)-N-[(1S)-1-(3-methoxyphenyl)ethyl]-2-methylpyrimidin-4-amine # PATENT #
Inhibitors of inducible form of 6-phosphofructose-2-kinase # EP-2702043-A1
CHEMBL3728189 # 2-[3-(1-{[6-(2H-1,3-benzodioxol-5-yl)-2-methylpyrimidin-4-yl]amino}ethyl)phenoxy]-N-
methylacetamide # PATENT # Inhibitors of inducible form of 6-phosphofructose-2-kinase # EP-2702043-A1
CHEMBL3728195 # 2-methyl-6-(1-methyl-1H-1,3-benzodiazol-6-yl)-N-[(1R)-1,2,3,4-tetrahydronaphthalen-1-yl]pyrimidin-
4-amine # PATENT # Inhibitors of inducible form of 6-phosphofructose-2-kinase # EP-2702043-A1
CHEMBL3728209 # 6-(3-amino-4-methylphenyl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3728213 # 2-[3-(1-{[6-(1,3-benzothiazol-6-yl)-2-methylpyrimidin-4-yl]amino}ethyl)phenoxy]-N-propylacetamide #
PATENT # Inhibitors of inducible form of 6-phosphofructose-2-kinase # EP-2702043-A1
CHEMBL3728216 # 2-[3-(1-{[6-(1,3-benzothiazol-6-yl)-2-methylpyrimidin-4-yl]amino}ethyl)phenoxy]-N-methylacetamide #
PATENT # Inhibitors of inducible form of 6-phosphofructose-2-kinase # EP-2702043-A1
CHEMBL3728255 # 2-[3-(1-{[6-(1,3-benzothiazol-6-yl)-2-methylpyrimidin-4-yl]amino}ethyl)phenoxy]acetamide # PATENT
# Inhibitors of inducible form of 6-phosphofructose-2-kinase # EP-2702043-A1
CHEMBL3728264 # 2-methyl-6-(quinolin-6-yl)-N-[(1R)-1,2,3,4-tetrahydronaphthalen-1-yl]pyrimidin-4-amine # PATENT #
Inhibitors of inducible form of 6-phosphofructose-2-kinase # EP-2702043-A1
CHEMBL3728296 # 5-{3-[(1S)-1-{[2-methyl-6-(3-methyl-1-benzofuran-5-yl)pyrimidin-4-yl]amino}ethyl]phenyl}pyridine-3-
carbonitrile # PATENT # Inhibitors of inducible form of 6-phosphofructose-2-kinase # EP-2702043-A1
CHEMBL3728357 # N,N-diethyl-2-[3-(1-{[2-methyl-6-(1-methyl-1H-indol-6-yl)pyrimidin-4-
yl]amino}ethyl)phenoxy]acetamide # PATENT # Inhibitors of inducible form of 6-phosphofructose-2-kinase # EP-
2702043-A1
CHEMBL3728363 # 2-methoxy-4-(2-methyl-6-{[(1R)-1,2,3,4-tetrahydronaphthalen-1-yl]amino}pyrimidin-4-yl)benzamide #
PATENT # Inhibitors of inducible form of 6-phosphofructose-2-kinase # EP-2702043-A1
CHEMBL3728403 # 2-methyl-6-(2-methylphenyl)-N-[(1R)-1,2,3,4-tetrahydronaphthalen-1-yl]pyrimidin-4-amine # PATENT
# Inhibitors of inducible form of 6-phosphofructose-2-kinase # EP-2702043-A1
CHEMBL3728409 # N-[(1S)-1-[3-(2-aminopyrimidin-5-yl)phenyl]ethyl]-6-(7-fluoro-3-methyl-1-benzofuran-5-yl)-2-
methylpyrimidin-4-amine # PATENT # Inhibitors of inducible form of 6-phosphofructose-2-kinase # EP-2702043-A1
CHEMBL3728410 # 2-methyl-6-(1-methyl-1H-indol-2-yl)-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3728422 # 6-(7-fluoro-3-methyl-1-benzofuran-5-yl)-2-methyl-N-{1-[3-(1-methyl-1H-pyrazol-4-
yl)phenyl]ethyl}pyrimidin-4-amine # PATENT # Inhibitors of inducible form of 6-phosphofructose-2-kinase # EP-2702043-
A1
CHEMBL3728426 # N-[(1S)-1-(3-bromophenyl)ethyl]-2-methyl-6-(3-methyl-1-benzothiophen-5-yl)pyrimidin-4-amine #
PATENT # Inhibitors of inducible form of 6-phosphofructose-2-kinase # EP-2702043-A1
CHEMBL3728457 # N-[(1S)-1-(3-methoxyphenyl)ethyl]-2-methyl-6-(1-methyl-1H-indol-6-yl)pyrimidin-4-amine # PATENT
# Inhibitors of inducible form of 6-phosphofructose-2-kinase # EP-2702043-A1
CHEMBL3728460 # 6-(1,3-benzothiazol-6-yl)-2-methyl-N-[(1S)-1-[3-(1-methyl-1H-pyrazol-4-yl)phenyl]ethyl]pyrimidin-4-
amine # PATENT # Inhibitors of inducible form of 6-phosphofructose-2-kinase # EP-2702043-A1
CHEMBL3728497 # 6-(4-chloro-3-fluorophenyl)-N-{1-[5-(1-ethyl-1H-pyrazol-4-yl)pyridin-3-yl]ethyl}-2-methylpyrimidin-4-
amine # PATENT # Inhibitors of inducible form of 6-phosphofructose-2-kinase # EP-2702043-A1
CHEMBL3728502 # 2-methyl-6-(1-methyl-1H-indol-6-yl)-N-[(1S)-1-phenylethyl]pyrimidin-4-amine # PATENT # Inhibitors
of inducible form of 6-phosphofructose-2-kinase # EP-2702043-A1
CHEMBL3728510 # 6-(1,3-benzothiazol-6-yl)-N-[(2-fluorophenyl)methyl]-2-methylpyrimidin-4-amine # PATENT #
Inhibitors of inducible form of 6-phosphofructose-2-kinase # EP-2702043-A1
CHEMBL3728524 # 6-(1,3-benzothiazol-6-yl)-N-(2,3-dihydro-1H-inden-2-yl)-2-methylpyrimidin-4-amine # PATENT #
Inhibitors of inducible form of 6-phosphofructose-2-kinase # EP-2702043-A1
CHEMBL3728534 # 6-(2,3-dihydro-1-benzofuran-5-yl)-2-methyl-N-[(1R)-1,2,3,4-tetrahydronaphthalen-1-yl]pyrimidin-4-
amine # PATENT # Inhibitors of inducible form of 6-phosphofructose-2-kinase # EP-2702043-A1
CHEMBL3728539 # 6-(1,3-benzothiazol-6-yl)-N-[(2-chlorophenyl)methyl]-2-methylpyrimidin-4-amine # PATENT #
Inhibitors of inducible form of 6-phosphofructose-2-kinase # EP-2702043-A1
CHEMBL3728545 # 2-methyl-6-(3-methyl-1-benzofuran-5-yl)-N-[(1S)-1-{3-[2-(methylamino)pyrimidin-5-
yl]phenyl}ethyl]pyrimidin-4-amine # PATENT # Inhibitors of inducible form of 6-phosphofructose-2-kinase # EP-2702043-
A1
CHEMBL3728550 # 2-methyl-6-(3-methyl-1-benzothiophen-5-yl)-N-[(1S)-1-[3-(1-methyl-1H-pyrazol-4-
yl)phenyl]ethyl]pyrimidin-4-amine # PATENT # Inhibitors of inducible form of 6-phosphofructose-2-kinase # EP-2702043-
A1
CHEMBL3728564 # 6-(4-chloro-3-methylphenyl)-N-[(4R)-3,4-dihydro-2H-1-benzopyran-4-yl]-2-methylpyrimidin-4-amine #
PATENT # Inhibitors of inducible form of 6-phosphofructose-2-kinase # EP-2702043-A1
CHEMBL3728565 # 6-(1,3-benzothiazol-6-yl)-N-{1-[3-(benzyloxy)phenyl]ethyl}-2-methylpyrimidin-4-amine # PATENT #
Inhibitors of inducible form of 6-phosphofructose-2-kinase # EP-2702043-A1
CHEMBL3728574 # 6-(2-chloro-3-methoxyphenyl)-N-(1-{3-[(1,3-dimethyl-1H-pyrazol-5-yl)methoxy]phenyl}ethyl)-2-
methylpyrimidin-4-amine # PATENT # Inhibitors of inducible form of 6-phosphofructose-2-kinase # EP-2702043-A1
CHEMBL3728575 # 2-methyl-6-(1-methyl-1H-indol-6-yl)-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3728583 # 2-methyl-6-(3-methyl-1-benzofuran-5-yl)-N-(1-{3-[(1-methyl-1H-pyrazol-3-
yl)methoxy]phenyl}ethyl)pyrimidin-4-amine # PATENT # Inhibitors of inducible form of 6-phosphofructose-2-kinase # EP-
2702043-A1
CHEMBL3728590 # N-[(1S)-1-[5-(1-ethyl-1H-pyrazol-4-yl)pyridin-3-yl]ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3728592 # N-methyl-2-[3-(1-{[2-methyl-6-(1-methyl-1H-indol-6-yl)pyrimidin-4-yl]amino}ethyl)phenoxy]acetamide
# PATENT # Inhibitors of inducible form of 6-phosphofructose-2-kinase # EP-2702043-A1
CHEMBL3728598 # N-[1-(3-bromo-4-fluorophenyl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3728602 # N-[(1S)-1-[3-(5-aminopyridin-3-yl)phenyl]ethyl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3728614 # N-[(1S)-1-[3-(5-aminopyridin-3-yl)phenyl]ethyl]-6-(7-fluoro-3-methyl-1-benzofuran-5-yl)-2-
methylpyrimidin-4-amine # PATENT # Inhibitors of inducible form of 6-phosphofructose-2-kinase # EP-2702043-A1
CHEMBL3728630 # 5-[3-(1-{[6-(1,3-benzothiazol-6-yl)-2-methylpyrimidin-4-yl]amino}ethyl)phenyl]pyrimidine-2-carbonitrile
# PATENT # Inhibitors of inducible form of 6-phosphofructose-2-kinase # EP-2702043-A1
CHEMBL3728631 # 6-(1,3-benzothiazol-6-yl)-2-methyl-N-(1-{3-[(pyridin-3-yl)methoxy]phenyl}ethyl)pyrimidin-4-amine #
PATENT # Inhibitors of inducible form of 6-phosphofructose-2-kinase # EP-2702043-A1
CHEMBL3728632 # 6-(1,3-benzothiazol-6-yl)-N-(2-ethylhexyl)-2-methylpyrimidin-4-amine # PATENT # Inhibitors of
inducible form of 6-phosphofructose-2-kinase # EP-2702043-A1
CHEMBL3728659 # N-[(1S)-1-[3-(2-aminopyrimidin-5-yl)phenyl]ethyl]-6-(4-chlorophenyl)-2-methylpyrimidin-4-amine #
PATENT # Inhibitors of inducible form of 6-phosphofructose-2-kinase # EP-2702043-A1
CHEMBL3728671 # N-(1-{3-[(1,3-dimethyl-1H-pyrazol-5-yl)methoxy]phenyl}ethyl)-6-(3-methoxy-4-methylphenyl)-2-
methylpyrimidin-4-amine # PATENT # Inhibitors of inducible form of 6-phosphofructose-2-kinase # EP-2702043-A1
CHEMBL3728687 # 6-(4-chlorophenyl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3728692 # 1-({[3-(1-{[6-(1,3-benzothiazol-6-yl)-2-methylpyrimidin-4-
yl]amino}ethyl)phenyl]carbamoyl}amino)formamide # PATENT # Inhibitors of inducible form of 6-phosphofructose-2-
kinase # EP-2702043-A1
CHEMBL3728696 # 6-(1,3-benzothiazol-6-yl)-2-methyl-N-{1-[3-(2-methyl-1,3-thiazol-4-yl)phenyl]ethyl}pyrimidin-4-amine
# PATENT # Inhibitors of inducible form of 6-phosphofructose-2-kinase # EP-2702043-A1
CHEMBL3728705 # N-[(1S)-1-(3-{2-[4-(2-methoxyethyl)piperazin-1-yl]pyrimidin-5-yl}phenyl)ethyl]-2-methyl-6-(3-methyl-1-
benzofuran-5-yl)pyrimidin-4-amine # PATENT # Inhibitors of inducible form of 6-phosphofructose-2-kinase # EP-
2702043-A1
CHEMBL3728709 # 2-[3-(1-{[2-methyl-6-(1-methyl-1H-indol-6-yl)pyrimidin-4-yl]amino}ethyl)phenoxy]-1-[4-(pyridin-2-
yl)piperazin-1-yl]ethan-1-one # PATENT # Inhibitors of inducible form of 6-phosphofructose-2-kinase # EP-2702043-A1
CHEMBL3728738 # 3-(1-{[6-(1,3-benzothiazol-6-yl)-2-methylpyrimidin-4-yl]amino}ethyl)-N-(prop-2-yn-1-yl)benzene-1-
sulfonamide # PATENT # Inhibitors of inducible form of 6-phosphofructose-2-kinase # EP-2702043-A1
CHEMBL3728755 # 6-(1,3-benzothiazol-6-yl)-2-methyl-N-[2-(pyridin-3-yl)ethyl]pyrimidin-4-amine # PATENT # Inhibitors
of inducible form of 6-phosphofructose-2-kinase # EP-2702043-A1
CHEMBL3728767 # N-{1-[5-(1-ethyl-1H-pyrazol-4-yl)pyridin-3-yl]ethyl}-6-(7-fluoro-3-methyl-1-benzofuran-5-yl)-2-
methylpyrimidin-4-amine # PATENT # Inhibitors of inducible form of 6-phosphofructose-2-kinase # EP-2702043-A1
CHEMBL3728770 # 3-[(1S)-1-{[2-methyl-6-(3-methyl-1-benzofuran-5-yl)pyrimidin-4-yl]amino}ethyl]phenol # PATENT #
Inhibitors of inducible form of 6-phosphofructose-2-kinase # EP-2702043-A1
CHEMBL3728785 # N-[(1S)-1-(3-methoxyphenyl)ethyl]-2-methyl-6-(1-methyl-1H-indol-2-yl)pyrimidin-4-amine # PATENT
# Inhibitors of inducible form of 6-phosphofructose-2-kinase # EP-2702043-A1
CHEMBL3728792 # 5-[3-(1-{[6-(1,3-benzothiazol-6-yl)-2-methylpyrimidin-4-yl]amino}ethyl)phenyl]pyridine-3-carbonitrile #
PATENT # Inhibitors of inducible form of 6-phosphofructose-2-kinase # EP-2702043-A1
CHEMBL3728794 # 2-methyl-6-(3-methyl-1-benzofuran-5-yl)-N-[(1S)-1-{3-[2-(piperazin-1-yl)pyrimidin-5-
yl]phenyl}ethyl]pyrimidin-4-amine # PATENT # Inhibitors of inducible form of 6-phosphofructose-2-kinase # EP-2702043-
A1
CHEMBL3728795 # (5-{3-[(1S)-1-{[6-(1,3-benzothiazol-6-yl)-2-methylpyrimidin-4-yl]amino}ethyl]phenyl}pyridin-3-
yl)methanol # PATENT # Inhibitors of inducible form of 6-phosphofructose-2-kinase # EP-2702043-A1
CHEMBL3728799 # 5-[3-(1-{[6-(1,3-benzothiazol-6-yl)-2-methylpyrimidin-4-yl]amino}ethyl)phenyl]pyridine-3-carboxamide
# PATENT # Inhibitors of inducible form of 6-phosphofructose-2-kinase # EP-2702043-A1
CHEMBL3728811 # N-(1-{3-[2-(dimethylamino)ethoxy]phenyl}ethyl)-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3728820 # 6-(1,3-benzothiazol-6-yl)-2-methyl-N-(pentan-2-yl)pyrimidin-4-amine # PATENT # Inhibitors of
inducible form of 6-phosphofructose-2-kinase # EP-2702043-A1
CHEMBL3728827 # 6-(3,4-dimethoxyphenyl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3728831 # 6-(7-fluoro-3-methyl-1-benzofuran-5-yl)-2-methyl-N-[(1S)-1-[3-(1-methyl-1H-pyrazol-4-
yl)phenyl]ethyl]pyrimidin-4-amine # PATENT # Inhibitors of inducible form of 6-phosphofructose-2-kinase # EP-2702043-
A1
CHEMBL3728832 # N-[(1S)-1-[3-(5-amino-6-chloropyridin-3-yl)phenyl]ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3728839 # 2-fluoro-4-(2-methyl-6-{[(1R)-1,2,3,4-tetrahydronaphthalen-1-yl]amino}pyrimidin-4-yl)benzamide #
PATENT # Inhibitors of inducible form of 6-phosphofructose-2-kinase # EP-2702043-A1
CHEMBL3728840 # 2-methyl-6-(3-methyl-1-benzofuran-5-yl)-N-[(1S)-1-{3-[6-(piperazin-1-yl)pyridin-3-
yl]phenyl}ethyl]pyrimidin-4-amine # PATENT # Inhibitors of inducible form of 6-phosphofructose-2-kinase # EP-2702043-
A1
CHEMBL3728844 # 2-methyl-N-{1-[3-(1-methyl-1H-pyrazol-4-yl)phenyl]ethyl}-6-(4-methyl-3,4-dihydro-2H-1,4-
benzoxazin-6-yl)pyrimidin-4-amine # PATENT # Inhibitors of inducible form of 6-phosphofructose-2-kinase # EP-
2702043-A1
CHEMBL3728885 # 6-(1,3-benzothiazol-6-yl)-2-methyl-N-(1-{3-[(1-methyl-1H-pyrazol-3-
yl)methoxy]phenyl}ethyl)pyrimidin-4-amine # PATENT # Inhibitors of inducible form of 6-phosphofructose-2-kinase # EP-
2702043-A1
CHEMBL3728904 # N-{1-[4-fluoro-3-(1-methyl-1H-pyrazol-4-yl)phenyl]ethyl}-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3728910 # 6-(1,3-benzothiazol-6-yl)-N-[(1S)-1-[3-(1-ethyl-1H-pyrazol-4-yl)phenyl]ethyl]-2-methylpyrimidin-4-
amine # PATENT # Inhibitors of inducible form of 6-phosphofructose-2-kinase # EP-2702043-A1
CHEMBL3728911 # 6-chloro-5′-[(1S)-1-{[2-methyl-6-(3-methyl-1-benzofuran-5-yl)pyrimidin-4-yl]amino}ethyl]-[3,3′-
bipyridin]-5-amine # PATENT # Inhibitors of inducible form of 6-phosphofructose-2-kinase # EP-2702043-A1
CHEMBL3728912 # 6-(1,3-benzothiazol-6-yl)-N-(1-{3-[(1,3-dimethyl-1H-pyrazol-5-yl)methoxy]phenyl}ethyl)-2-
methylpyrimidin-4-amine # PATENT # Inhibitors of inducible form of 6-phosphofructose-2-kinase # EP-2702043-A1
CHEMBL3728919 # 6-(1,3-benzothiazol-6-yl)-2-methyl-N-(2-methylbutyl)pyrimidin-4-amine # PATENT # Inhibitors of
inducible form of 6-phosphofructose-2-kinase # EP-2702043-A1
CHEMBL3728922 # 1-hydroxy-3-[(5-{3-[(1S)-1-{[2-methyl-6-(3-methyl-1-benzofuran-5-yl)pyrimidin-4-
yl]amino}ethyl]phenyl}pyrimidin-2-yl)amino]propan-2-one # PATENT # Inhibitors of inducible form of 6-phosphofructose-
2-kinase # EP-2702043-A1
CHEMBL3728933 # 2-methyl-6-(3-methyl-1-benzofuran-5-yl)-N-[(1S)-1-{3-[6-(morpholin-4-yl)pyridin-3-
yl]phenyl}ethyl]pyrimidin-4-amine # PATENT # Inhibitors of inducible form of 6-phosphofructose-2-kinase # EP-2702043-
A1
CHEMBL3728952 # 6-(1,3-benzothiazol-6-yl)-2-methyl-N-{1-[3-(1H-1,2,3-triazol-1-yl)phenyl]ethyl}pyrimidin-4-amine #
PATENT # Inhibitors of inducible form of 6-phosphofructose-2-kinase # EP-2702043-A1
CHEMBL3728960 # 1-(azetidin-1-yl)-2-[3-(1-{[6-(1,3-benzothiazol-6-yl)-2-methylpyrimidin-4-
yl]amino}ethyl)phenoxy]ethan-1-one # PATENT # Inhibitors of inducible form of 6-phosphofructose-2-kinase # EP-
2702043-A1
CHEMBL3728964 # N-(5-{[6-(1,3-benzothiazol-6-yl)-2-methylpyrimidin-4-yl]amino}-5,6,7,8-tetrahydronaphthalen-2-
yl)acetamide # PATENT # Inhibitors of inducible form of 6-phosphofructose-2-kinase # EP-2702043-A1
CHEMBL3728971 # N-{1-[3-(2-aminopyrimidin-5-yl)-5-fluorophenyl]ethyl}-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3729008 # 2-[3-(1-{[6-(2-chloro-3-methoxyphenyl)-2-methylpyrimidin-4-yl]amino}ethyl)phenoxy]-N-
methylacetamide # PATENT # Inhibitors of inducible form of 6-phosphofructose-2-kinase # EP-2702043-A1
CHEMBL3729009 # 2-methyl-6-(3-methyl-1-benzofuran-5-yl)-N-[(1S)-1-[3-(6-methylpyridin-3-yl)phenyl]ethyl]pyrimidin-4-
amine # PATENT # Inhibitors of inducible form of 6-phosphofructose-2-kinase # EP-2702043-A1
CHEMBL3729017 # 6-(4-chloro-3,5-difluorophenyl)-2-methyl-N-{1-[3-(1-methyl-1H-pyrazol-4-yl)phenyl]ethyl}pyrimidin-4-
amine # PATENT # Inhibitors of inducible form of 6-phosphofructose-2-kinase # EP-2702043-A1
CHEMBL3729019 # 6-(3-ethyl-1-benzofuran-5-yl)-2-methyl-N-[(1R)-1,2,3,4-tetrahydronaphthalen-1-yl]pyrimidin-4-amine
# PATENT # Inhibitors of inducible form of 6-phosphofructose-2-kinase # EP-2702043-A1
CHEMBL3729020 # 6-(1-benzothiophen-2-yl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3729026 # 6-(1,3-benzothiazol-6-yl)-N-{1-[3-(3,5-dimethyl-1,2-oxazol-4-yl)phenyl]ethyl}-2-methylpyrimidin-4-
amine # PATENT # Inhibitors of inducible form of 6-phosphofructose-2-kinase # EP-2702043-A1
CHEMBL3729035 # N-{1-[3-(2-aminopyrimidin-5-yl)-4-fluorophenyl]ethyl}-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3729039 # 2-chloro-5-(2-methyl-6-{[(1R)-1,2,3,4-tetrahydronaphthalen-1-yl]amino}pyrimidin-4-yl)benzonitrile #
PATENT # Inhibitors of inducible form of 6-phosphofructose-2-kinase # EP-2702043-A1
CHEMBL3729048 # 2-[3-(1-{[2-methyl-6-(3-methyl-1-benzofuran-5-yl)pyrimidin-4-yl]amino}ethyl)phenoxy]ethan-1-ol #
PATENT # Inhibitors of inducible form of 6-phosphofructose-2-kinase # EP-2702043-A1
CHEMBL3729056 # methyl 1-{2-[3-(1-{[2-methyl-6-(1-methyl-1H-indol-6-yl)pyrimidin-4-
yl]amino}ethyl)phenoxy]acetyl}piperidine-4-carboxylate # PATENT # Inhibitors of inducible form of 6-phosphofructose-2-
kinase # EP-2702043-A1
CHEMBL3729061 # 2-methyl-6-(2-phenylethyl)-N-[(1R)-1,2,3,4-tetrahydronaphthalen-1-yl]pyrimidin-4-amine # PATENT #
Inhibitors of inducible form of 6-phosphofructose-2-kinase # EP-2702043-A1
CHEMBL3729068 # 6-(3-chloro-4-methylphenyl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3729096 # 6-(2,3-dihydro-1,4-benzodioxin-6-yl)-2-methyl-N-{1-[3-(1-methyl-1H-pyrazol-4-
yl)phenyl]ethyl}pyrimidin-4-amine # PATENT # Inhibitors of inducible form of 6-phosphofructose-2-kinase # EP-2702043-
A1
CHEMBL3729097 # 6-(1-benzofuran-5-yl)-N-[(1S)-1-(3-methoxyphenyl)ethyl]-2-methylpyrimidin-4-amine # PATENT #
Inhibitors of inducible form of 6-phosphofructose-2-kinase # EP-2702043-A1
CHEMBL3729110 # N-{1-[3-(5-amino-6-chloropyridin-3-yl)-5-fluorophenyl]ethyl}-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3729124 # 6-(1,3-benzothiazol-6-yl)-N-{1-[3-(4-chloropyridin-3-yl)phenyl]ethyl}-2-methylpyrimidin-4-amine #
PATENT # Inhibitors of inducible form of 6-phosphofructose-2-kinase # EP-2702043-A1
CHEMBL3729134 # N-[1-(3-aminophenyl)ethyl]-6-(1,3-benzothiazol-6-yl)-2-methylpyrimidin-4-amine # PATENT #
Inhibitors of inducible form of 6-phosphofructose-2-kinase # EP-2702043-A1
CHEMBL3729142 # N-[(1S)-1-{3-[2-(dimethylamino)pyrimidin-5-yl]phenyl}ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3729147 # 6-(1,3-benzothiazol-6-yl)-2-methyl-N-[(1S)-1-[3-(1-methyl-1H-pyrrol-2-yl)phenyl]ethyl]pyrimidin-4-
amine # PATENT # Inhibitors of inducible form of 6-phosphofructose-2-kinase # EP-2702043-A1
CHEMBL3729185 # 6-(2H-1,3-benzodioxol-5-yl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3729193 # 6-(1,3-benzothiazol-6-yl)-N-(1-{3-[(1-ethylpiperidin-3-yl)methoxy]phenyl}ethyl)-2-methylpyrimidin-4-
amine # PATENT # Inhibitors of inducible form of 6-phosphofructose-2-kinase # EP-2702043-A1
CHEMBL3729219 # 2-[(5-{3-[(1S)-1-{[2-methyl-6-(3-methyl-1-benzofuran-5-yl)pyrimidin-4-yl]amino}ethyl]phenyl}pyridin-2-
yl)amino]ethan-1-ol # PATENT # Inhibitors of inducible form of 6-phosphofructose-2-kinase # EP-2702043-A1
CHEMBL3729244 # 6-(1,3-benzothiazol-6-yl)-N-{1-[3-(2-fluoropyrimidin-5-yl)phenyl]ethyl}-2-methylpyrimidin-4-amine #
PATENT # Inhibitors of inducible form of 6-phosphofructose-2-kinase # EP-2702043-A1
CHEMBL3729249 # 6-(1,3-benzothiazol-6-yl)-N-(6-fluoro-3,4-dihydro-2H-1-benzopyran-4-yl)-2-methylpyrimidin-4-amine
# PATENT # Inhibitors of inducible form of 6-phosphofructose-2-kinase # EP-2702043-A1
CHEMBL3729255 # N-[(1S)-1-[3-(2-aminopyrimidin-5-yl)phenyl]ethyl]-6-(1,3-benzothiazol-6-yl)-2-methylpyrimidin-4-
amine # PATENT # Inhibitors of inducible form of 6-phosphofructose-2-kinase # EP-2702043-A1
CHEMBL3729257 # 2-[(5-{3-[(1S)-1-{[2-methyl-6-(3-methyl-1-benzofuran-5-yl)pyrimidin-4-
yl]amino}ethyl]phenyl}pyrimidin-2-yl)amino]ethan-1-ol # PATENT # Inhibitors of inducible form of 6-phosphofructose-2-
kinase # EP-2702043-A1
CHEMBL3729266 # N,N-diethyl-2-[3-(1-{[2-methyl-6-(4-methyl-3,4-dihydro-2H-1,4-benzoxazin-6-yl)pyrimidin-4-
yl]amino}ethyl)phenoxy]acetamide # PATENT # Inhibitors of inducible form of 6-phosphofructose-2-kinase # EP-
2702043-A1
CHEMBL3729286 # 3-(1-{[6-(1,3-benzothiazol-6-yl)-2-methylpyrimidin-4-yl]amino}ethyl)benzene-1-sulfonamide #
PATENT # Inhibitors of inducible form of 6-phosphofructose-2-kinase # EP-2702043-A1
CHEMBL3729294 # 2-methyl-6-(3-methyl-1-benzofuran-5-yl)-N-[(1S)-1-(4-methylphenyl)ethyl]pyrimidin-4-amine #
PATENT # Inhibitors of inducible form of 6-phosphofructose-2-kinase # EP-2702043-A1
CHEMBL3729329 # N-[(1S)-1-[3-(5-amino-6-chloropyridin-3-yl)phenyl]ethyl]-6-(7-fluoro-3-methyl-1-benzofuran-5-yl)-2-
methylpyrimidin-4-amine # PATENT # Inhibitors of inducible form of 6-phosphofructose-2-kinase # EP-2702043-A1
CHEMBL3729334 # 6-(1,3-benzothiazol-6-yl)-2-methyl-N-[1-(3-nitrophenyl)ethyl]pyrimidin-4-amine # PATENT #
Inhibitors of inducible form of 6-phosphofructose-2-kinase # EP-2702043-A1
CHEMBL3729335 # 3-{3-[(1S)-1-{[6-(1,3-benzothiazol-6-yl)-2-methylpyrimidin-4-yl]amino}ethyl]phenyl}-N-tert-butylprop-
2-enamide # PATENT # Inhibitors of inducible form of 6-phosphofructose-2-kinase # EP-2702043-A1
CHEMBL3729349 # N-[(1S)-1-[3-(2-aminopyrimidin-5-yl)phenyl]ethyl]-2-methyl-6-(1-methyl-1H-indol-6-yl)pyrimidin-4-
amine # PATENT # Inhibitors of inducible form of 6-phosphofructose-2-kinase # EP-2702043-A1
CHEMBL3729361 # 2-methyl-6-(1-methyl-2,3-dihydro-1H-indol-6-yl)-N-[(1R)-1,2,3,4-tetrahydronaphthalen-1-y l]pyrimidin-
4-amine # PATENT # Inhibitors of inducible form of 6-phosphofructose-2-kinase # EP-2702043-A1
CHEMBL3729363 # 3-(1-{[6-(1,3-benzothiazol-6-yl)-2-methylpyrimidin-4-yl]amino}ethyl)benzonitrile # PATENT #
Inhibitors of inducible form of 6-phosphofructose-2-kinase # EP-2702043-A1
CHEMBL3729381 # N-{1-[5-(2-aminopyrimidin-5-yl)pyridin-3-yl]ethyl}-6-(7-fluoro-3-methyl-1-benzofuran-5-yl)-2-
methylpyrimidin-4-amine # PATENT # Inhibitors of inducible form of 6-phosphofructose-2-kinase # EP-2702043-A1
CHEMBL3729404 # 2-methyl-6-(3-methyl-1-benzofuran-5-yl)-N-[(1R)-1,2,3,4-tetrahydronaphthalen-1-yl]pyrimidin-4-
amine # PATENT # Inhibitors of inducible form of 6-phosphofructose-2-kinase # EP-2702043-A1
CHEMBL3729413 # 6-(4-chloro-3-fluorophenyl)-2-methyl-N-[(1S)-1-[3-(1-methyl-1H-pyrazol-4-yl)phenyl]ethyl]pyrimidin-4-
amine # PATENT # Inhibitors of inducible form of 6-phosphofructose-2-kinase # EP-2702043-A1
CHEMBL3729426 # 6-(1,3-benzothiazol-6-yl)-N-{1-[4-fluoro-3-(1-methyl-1H-pyrazol-4-yl)phenyl]ethyl}-2-methylpyrimidin-
4-amine # PATENT # Inhibitors of inducible form of 6-phosphofructose-2-kinase # EP-2702043-A1
CHEMBL3729430 # 6-(2H-1,3-benzodioxol-5-yl)-N-[(1S)-1-(3-methoxyphenyl)ethyl]-2-methylpyrimidin-4-amine #
PATENT # Inhibitors of inducible form of 6-phosphofructose-2-kinase # EP-2702043-A1
CHEMBL3729446 # 6-(7-fluoro-1-benzofuran-5-yl)-N-[(1S)-1-(3-methoxyphenyl)ethyl]-2-methylpyrimidin-4-amine #
PATENT # Inhibitors of inducible form of 6-phosphofructose-2-kinase # EP-2702043-A1
CHEMBL3729498 # 6-(1,3-benzothiazol-6-yl)-2-methyl-N-{1-[3-(pyridin-3-yl)phenyl]ethyl}pyrimidin-4-amine # PATENT #
Inhibitors of inducible form of 6-phosphofructose-2-kinase # EP-2702043-A1
CHEMBL3729518 # 6-(1,3-benzothiazol-6-yl)-2-methyl-N-(1-{3-[(2-methyl-1,3-thiazol-4-
yl)methoxy]phenyl}ethyl)pyrimidin-4-amine # PATENT # Inhibitors of inducible form of 6-phosphofructose-2-kinase # EP-
2702043-A1
CHEMBL3729521 # N-{1-[3-(6-aminopyridin-3-yl)phenyl]ethyl}-6-(1,3-benzothiazol-6-yl)-2-methylpyrimidin-4-amine #
PATENT # Inhibitors of inducible form of 6-phosphofructose-2-kinase # EP-2702043-A1
CHEMBL3729533 # 1-[4-(5-{3-[(1S)-1-{[2-methyl-6-(3-methyl-1-benzofuran-5-yl)pyrimidin-4-
yl]amino}ethyl]phenyl}pyrimidin-2-yl)piperazin-1-yl]ethan-1-one # PATENT # Inhibitors of inducible form of 6-
phosphofructose-2-kinase # EP-2702043-A1
CHEMBL3729536 # 2-[(5-{3-[(1S)-1-{[2-methyl-6-(3-methyl-1-benzofuran-5-yl)pyrimidin-4-
yl]amino}ethyl]phenyl}pyrimidin-2-yl)amino]acetic acid # PATENT # Inhibitors of inducible form of 6-phosphofructose-2-
kinase # EP-2702043-A1
CHEMBL3729549 # 6-(1,3-benzothiazol-6-yl)-2-methyl-N-(1-{3-[(1-methylpiperidin-3-yl)methoxy]phenyl}ethyl)pyrimidin-4-
amine # PATENT # Inhibitors of inducible form of 6-phosphofructose-2-kinase # EP-2702043-A1
CHEMBL3729555 # 6-(1-benzofuran-2-yl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3729561 # 2-[3-(1-{[2-methyl-6-(1-methyl-1H-indol-6-yl)pyrimidin-4-yl]amino}ethyl)phenoxy]acetonitrile #
PATENT # Inhibitors of inducible form of 6-phosphofructose-2-kinase # EP-2702043-A1
CHEMBL3729592 # 6-(1,3-benzothiazol-6-yl)-N-{1-[3-(6-methoxypyridin-3-yl)phenyl]ethyl}-2-methylpyrimidin-4-amine #
PATENT # Inhibitors of inducible form of 6-phosphofructose-2-kinase # EP-2702043-A1
CHEMBL3729619 # 2-[3-(1-{[6-(1,3-benzothiazol-6-yl)-2-methylpyrimidin-4-yl]amino}ethyl)phenoxy]-1-(pyrrolidin-1-
yl)ethan-1-one # PATENT # Inhibitors of inducible form of 6-phosphofructose-2-kinase # EP-2702043-A1
CHEMBL3729623 # 2-methyl-6-(3-methyl-1-benzofuran-5-yl)-N-[(1R)-1-[3-(1-methyl-1H-pyrazol-4-
yl)phenyl]ethyl]pyrimidin-4-amine # PATENT # Inhibitors of inducible form of 6-phosphofructose-2-kinase # EP-2702043-
A1
CHEMBL3729625 # 3-(1-{[6-(3-ethyl-1-benzofuran-5-yl)-2-methylpyrimidin-4-yl]amino}ethyl)phenol # PATENT #
Inhibitors of inducible form of 6-phosphofructose-2-kinase # EP-2702043-A1
CHEMBL3729627 # N-{1-[3-(furan-3-yl)phenyl]ethyl}-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3729636 # 2-methyl-6-(quinolin-3-yl)-N-[(1R)-1,2,3,4-tetrahydronaphthalen-1-yl]pyrimidin-4-amine # PATENT #
Inhibitors of inducible form of 6-phosphofructose-2-kinase # EP-2702043-A1
CHEMBL3729648 # 6-(1,3-benzothiazol-6-yl)-2-methyl-N-(1-{3-[(5-methyl-1,2-oxazol-3-
yl)methoxy]phenyl}ethyl)pyrimidin-4-amine # PATENT # Inhibitors of inducible form of 6-phosphofructose-2-kinase # EP-
2702043-A1
CHEMBL3729661 # 6-(1,3-benzothiazol-6-yl)-N-[(1S)-1-(3-methoxyphenyl)ethyl]-2-methylpyrimidin-4-amine # PATENT #
Inhibitors of inducible form of 6-phosphofructose-2-kinase # EP-2702043-A1
CHEMBL3729670 # N-[(1S)-1-(3-methoxyphenyl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3729673 # 2-methyl-6-(quinoxalin-6-yl)-N-[(1R)-1,2,3,4-tetrahydronaphthalen-1-yl]pyrimidin-4-amine # PATENT
# Inhibitors of inducible form of 6-phosphofructose-2-kinase # EP-2702043-A1
CHEMBL3729675 # 6-(1-ethyl-1H-indol-6-yl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3729678 # 6-(1,3-benzothiazol-6-yl)-2-methyl-N-[(1S)-1-[3-(1H-pyrazol-3-yl)phenyl]ethyl]pyrimidin-4-amine #
PATENT # Inhibitors of inducible form of 6-phosphofructose-2-kinase # EP-2702043-A1
CHEMBL3729680 # 2-methyl-6-{4-methyl-3-[(prop-2-yn-1-yl)amino]phenyl}-N-[(1R)-1,2,3,4-tetrahydronaphthalen-1-
yl]pyrimidin-4-amine # PATENT # Inhibitors of inducible form of 6-phosphofructose-2-kinase # EP-2702043-A1
CHEMBL3729692 # 6-(2-fluoro-3-methoxyphenyl)-2-methyl-N-[(1R)-1,2,3,4-tetrahydronaphthalen-1-yl]pyrimidin-4-amine
# PATENT # Inhibitors of inducible form of 6-phosphofructose-2-kinase # EP-2702043-A1
CHEMBL3729702 # 6-(1,3-benzothiazol-6-yl)-2-methyl-N-[(1S)-1-{3-[2-(methylamino)pyrimidin-5-
yl]phenyl}ethyl]pyrimidin-4-amine # PATENT # Inhibitors of inducible form of 6-phosphofructose-2-kinase # EP-2702043-
A1
CHEMBL3729708 # tert-butyl 3-{[3-(1-{[2-methyl-6-(3-methyl-1-benzofuran-5-yl)pyrimidin-4-
yl]amino}ethyl)phenoxy]methyl}piperidine-1-carboxylate # PATENT # Inhibitors of inducible form of 6-phosphofructose-2-
kinase # EP-2702043-A1
CHEMBL3729716 # N-[6-(1,3-benzothiazol-6-yl)-2-methylpyrimidin-4-yl]-1-methyl-1,2,3,4-tetrahydroquinolin-4-amine #
PATENT # Inhibitors of inducible form of 6-phosphofructose-2-kinase # EP-2702043-A1
CHEMBL3729732 # 2-methyl-6-(3-methyl-1-benzofuran-5-yl)-N-[(1S)-1-[5-(1-methyl-1H-pyrazol-5-yl)pyridin-3-
yl]ethyl]pyrimidin-4-amine # PATENT # Inhibitors of inducible form of 6-phosphofructose-2-kinase # EP-2702043-A1
CHEMBL3729736 # 6-(7-fluoro-1-benzofuran-5-yl)-2-methyl-N-{1-[3-(1-methyl-1H-pyrazol-4-yl)phenyl]ethyl}pyrimidin-4-
amine # PATENT # Inhibitors of inducible form of 6-phosphofructose-2-kinase # EP-2702043-A1
CHEMBL3729750 # 6-(4-ethyl-3,4-dihydro-2H-1,4-benzoxazin-6-yl)-2-methyl-N-[(1R)-1,2,3,4-tetrahydronaphthalen-1-
yl]pyrimidin-4-amine # PATENT # Inhibitors of inducible form of 6-phosphofructose-2-kinase # EP-2702043-A1
CHEMBL3729759 # 6-(1,3-benzothiazol-6-yl)-2-methyl-N-{1-[3-(5-methyl-1,2,4-oxadiazol-3-yl)phenyl]ethyl}pyrimidin-4-
amine # PATENT # Inhibitors of inducible form of 6-phosphofructose-2-kinase # EP-2702043-A1
CHEMBL3729777 # 6-(3-amino-4-chlorophenyl)-N-[(4R)-3,4-dihydro-2H-1-benzopyran-4-yl]-2-methylpyrimidin-4-amine #
PATENT # Inhibitors of inducible form of 6-phosphofructose-2-kinase # EP-2702043-A1
CHEMBL3729778 # ethyl 2-(4-{3-[(1S)-1-{[2-methyl-6-(3-methyl-1-benzofuran-5-yl)pyrimidin-4-yl]amino}ethyl]phenyl}-1H-
pyrazol-1-yl)acetate # PATENT # Inhibitors of inducible form of 6-phosphofructose-2-kinase # EP-2702043-A1
CHEMBL3729779 # 6-[3-(dimethylamino)phenyl]-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3729789 # 3-[(1R)-1-{[2-methyl-6-(3-methyl-1-benzofuran-5-yl)pyrimidin-4-yl]amino}ethyl]phenol # PATENT #
Inhibitors of inducible form of 6-phosphofructose-2-kinase # EP-2702043-A1
CHEMBL3729799 # 2-[3-(1-{[6-(1,3-benzothiazol-6-yl)-2-methylpyrimidin-4-yl]amino}ethyl)phenoxy]-N-(2-
methoxyethyl)acetamide # PATENT # Inhibitors of inducible form of 6-phosphofructose-2-kinase # EP-2702043-A1
CHEMBL3729801 # 2-methyl-6-phenyl-N-[(1R)-1,2,3,4-tetrahydronaphthalen-1-yl]pyrimidin-4-amine # PATENT #
Inhibitors of inducible form of 6-phosphofructose-2-kinase # EP-2702043-A1
CHEMBL3729803 # 6-(1,3-benzothiazol-6-yl)-N-[(2-fluoro-5-methoxyphenyl)methyl]-2-methylpyrimidin-4-amine #
PATENT # Inhibitors of inducible form of 6-phosphofructose-2-kinase # EP-2702043-A1
CHEMBL3729814 # 2-methyl-6-(3-methyl-1-benzofuran-5-yl)-N-[(1S)-1-[3-(5-methylpyridin-3-yl)phenyl]ethyl]pyrimidin-4-
amine # PATENT # Inhibitors of inducible form of 6-phosphofructose-2-kinase # EP-2702043-A1
CHEMBL3729815 # 2-methyl-6-(1-methyl-1H-indazol-6-yl)-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3729824 # 3-{3-[(1S)-1-{[6-(1,3-benzothiazol-6-yl)-2-methylpyrimidin-4-yl]amino}ethyl]phenyl}-N-ethylprop-2-
enamide # PATENT # Inhibitors of inducible form of 6-phosphofructose-2-kinase # EP-2702043-A1
CHEMBL3729829 # N-[(4R)-3,4-dihydro-2H-1-benzopyran-4-yl]-2-methyl-6-(4-methyl-3,4-dihydro-2H-1,4-benzoxazin-6-
yl)pyrimidin-4-amine # PATENT # Inhibitors of inducible form of 6-phosphofructose-2-kinase # EP-2702043-A1
CHEMBL3729834 # 1-[3-(1-{[6-(1,3-benzothiazol-6-yl)-2-methylpyrimidin-4-yl]amino}ethyl)phenoxy]-2-methylpropan-2-ol
# PATENT # Inhibitors of inducible form of 6-phosphofructose-2-kinase # EP-2702043-A1
CHEMBL3729846 # 6-(1,3-benzothiazol-6-yl)-N-[(1S)-1-{3-[2-(4-ethylpiperazin-1-yl)pyrimidin-5-yl]phenyl}ethyl]-2-
methylpyrimidin-4-amine # PATENT # Inhibitors of inducible form of 6-phosphofructose-2-kinase # EP-2702043-A1
CHEMBL3729850 # 6-(1,3-benzothiazol-6-yl)-2-methyl-N-[(1S)-1-{3-[2-(piperidin-1-yl)pyrimidin-5-
yl]phenyl}ethyl]pyrimidin-4-amine # PATENT # Inhibitors of inducible form of 6-phosphofructose-2-kinase # EP-2702043-
A1
CHEMBL3729856 # 6-(1,3-benzothiazol-6-yl)-N-(1-{3-[2-(1H-imidazol-1-yl)ethoxy]phenyl}ethyl)-2-methylpyrimidin-4-
amine # PATENT # Inhibitors of inducible form of 6-phosphofructose-2-kinase # EP-2702043-A1
CHEMBL3729887 # 6-(1,3-benzothiazol-6-yl)-N-[(2-chloro-6-fluoro-3-methylphenyl)methyl]-2-methylpyrimidin-4-amine #
PATENT # Inhibitors of inducible form of 6-phosphofructose-2-kinase # EP-2702043-A1
CHEMBL3729898 # 6-(3-amino-4-chlorophenyl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3729903 # 2-{3-[(1R)-1-{[2-methyl-6-(3-methyl-1-benzofuran-5-yl)pyrimidin-4-yl]amino}ethyl]phenoxy}-1-
(morpholin-4-yl)ethan-1-one # PATENT # Inhibitors of inducible form of 6-phosphofructose-2-kinase # EP-2702043-A1
CHEMBL3729910 # N-{1-[3-fluoro-5-(1-methyl-1H-pyrazol-4-yl)phenyl]ethyl}-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3729912 # 6-(1,3-benzothiazol-6-yl)-N-{1-[3-(6-fluoro-2-methylpyridin-3-yl)phenyl]ethyl}-2-methylpyrimidin-4-
amine # PATENT # Inhibitors of inducible form of 6-phosphofructose-2-kinase # EP-2702043-A1
CHEMBL3729929 # 6-(1,3-benzothiazol-6-yl)-2-methyl-N-(1-{3-[5-(trifluoromethyl)pyridin-3-yl]phenyl}ethyl)pyrimidin-4-
amine # PATENT # Inhibitors of inducible form of 6-phosphofructose-2-kinase # EP-2702043-A1
CHEMBL3729932 # N-[(1S)-1-{3-[2-(4-fluoropiperidin-1-yl)pyrimidin-5-yl]phenyl}ethyl]-2-methyl-6-(3-methyl-1-
benzofuran-5-yl)pyrimidin-4-amine # PATENT # Inhibitors of inducible form of 6-phosphofructose-2-kinase # EP-
2702043-A1
CHEMBL3729935 # 2-[4-(5-{3-[(1S)-1-{[2-methyl-6-(3-methyl-1-benzofuran-5-yl)pyrimidin-4-
yl]amino}ethyl]phenyl}pyrimidin-2-yl)piperazin-1-yl]ethan-1-ol # PATENT # Inhibitors of inducible form of 6-
phosphofructose-2-kinase # EP-2702043-A1
CHEMBL3729936 # 4-(2-methyl-6-{[(1R)-1,2,3,4-tetrahydronaphthalen-1-yl]amino}pyrimidin-4-yl)phenol # PATENT #
Inhibitors of inducible form of 6-phosphofructose-2-kinase # EP-2702043-A1
CHEMBL3729949 # 2-methyl-6-(3-methyl-1-benzofuran-5-yl)-N-[(1S)-1-[3-(1H-pyrazol-4-yl)phenyl]ethyl]pyrimidin-4-
amine # PATENT # Inhibitors of inducible form of 6-phosphofructose-2-kinase # EP-2702043-A1
CHEMBL3729963 # 6-(7-fluoro-3-methyl-1-benzofuran-5-yl)-2-methyl-N-{1-[5-(1H-pyrazol-4-yl)pyridin-3-
yl]ethyl}pyrimidin-4-amine # PATENT # Inhibitors of inducible form of 6-phosphofructose-2-kinase # EP-2702043-A1
CHEMBL3729968 # N-[1-(3-fluorophenyl)ethyl]-2-methyl-6-(3-methyl-1-benzofuran-5-yl)pyrimidin-4-amine # PATENT #
Inhibitors of inducible form of 6-phosphofructose-2-kinase # EP-2702043-A1
CHEMBL3729971 # 2-{3-[(1S)-1-{[2-methyl-6-(3-methyl-1-benzofuran-5-yl)pyrimidin-4-yl]amino}ethyl]phenoxy}-1-
(morpholin-4-yl)ethan-1-one # PATENT # Inhibitors of inducible form of 6-phosphofructose-2-kinase # EP-2702043-A1
CHEMBL3729973 # 3-(1-{[6-(1,3-benzothiazol-6-yl)-2-methylpyrimidin-4-yl]amino}ethyl)phenol # PATENT # Inhibitors of
inducible form of 6-phosphofructose-2-kinase # EP-2702043-A1
CHEMBL3729999 # 6-(1,3-benzothiazol-6-yl)-N-[(1S)-1-{3-[6-(4-ethylpiperazin-1-yl)pyridin-3-yl]phenyl}ethyl]-2-
methylpyrimidin-4-amine # PATENT # Inhibitors of inducible form of 6-phosphofructose-2-kinase # EP-2702043-A1
CHEMBL3730004 # 6-(4-ethylphenyl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3730020 # N-[6-(1,3-benzothiazol-6-yl)-2-methylpyrimidin-4-yl]-1,2,3,4-tetrahydroquinolin-4-amine # PATENT #
Inhibitors of inducible form of 6-phosphofructose-2-kinase # EP-2702043-A1
CHEMBL3730022 # 6-(1,3-benzothiazol-6-yl)-N-(3,4-dihydro-2H-1-benzopyran-4-yl)-2-methylpyrimidin-4-amine #
PATENT # Inhibitors of inducible form of 6-phosphofructose-2-kinase # EP-2702043-A1
CHEMBL3730028 # N-[(1S)-1-(4-methoxyphenyl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3730063 # 2-methyl-6-(3-methyl-1-benzofuran-5-yl)-N-[(1S)-1-[3-(1-methyl-1H-pyrazol-4-
yl)phenyl]ethyl]pyrimidin-4-amine # PATENT # Inhibitors of inducible form of 6-phosphofructose-2-kinase # EP-2702043-
A1
CHEMBL3730065 # 2-methyl-6-(3-methyl-1-benzofuran-5-yl)-N-[(1S)-1-{3-[2-(4-methylpiperazin-1-yl)pyrimidin-5-
yl]phenyl}ethyl]pyrimidin-4-amine # PATENT # Inhibitors of inducible form of 6-phosphofructose-2-kinase # EP-2702043-
A1
CHEMBL3730072 # 6-(4-methoxyphenyl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3730085 # 6-(3-fluorophenyl)-2-methyl-N-{1[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3730093 # 6-(1,3-benzothiazol-6-yl)-N-{1-[3-(methanesulfonylmethoxy)phenyl]ethyl}-2-methylpyrimidin-4-amine
# PATENT # Inhibitors of inducible form of 6-phosphofructose-2-kinase # EP-2702043-A1
CHEMBL3730098 # 3-[2-methyl-6-({1-[3-(1-methyl-1H-pyrazol-4-yl)phenyl]ethyl}amino)pyrimidin-4-yl]phenol # PATENT #
Inhibitors of inducible form of 6-phosphofructose-2-kinase # EP-2702043-A1
CHEMBL3730102 # 6-(1H-indol-5-yl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3730106 # 7-(2-methyl-6-{[(1R)-1,2,3,4-tetrahydronaphthalen-1-yl]amino}pyrimidin-4-yl)naphthalen-1-ol #
PATENT # Inhibitors of inducible form of 6-phosphofructose-2-kinase # EP-2702043-A1
CHEMBL3730107 # 2-{[5-(6-{[(4R)-3,4-di hydro-2H-1-benzopyran-4-yl]amino}-2-methylpyrimidin-4-yl)-2-
methylphenyl]amino}acetonitrile # PATENT # Inhibitors of inducible form of 6-phosphofructose-2-kinase # EP-2702043-
A1
CHEMBL3730109 # 2-methyl-6-(3-methyl-1-benzofuran-5-yl)-N-[(1S)-1-{6′-methyl-[3,3′-bipyridin]-5-yl}ethyl]pyrimidin-4-
amine # PATENT # Inhibitors of inducible form of 6-phosphofructose-2-kinase # EP-2702043-A1
CHEMBL3730111 # 6-(1,3-benzoxazol-6-yl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3730114 # 6-(1,3-benzothiazol-6-yl)-N-[(1S)-1-[3-(6-fluoropyridin-3-yl)phenyl]ethyl]-2-methylpyrimidin-4-amine #
PATENT # Inhibitors of inducible form of 6-phosphofructose-2-kinase # EP-2702043-A1
CHEMBL3730119 # (2S)-3-[(5-{3-[(1S)-1-{[2-methyl-6-(3-methyl-1-benzofuran-5-yl)pyrimidin-4-
yl]amino}ethyl]phenyl}pyrimidin-2-yl)amino]propane-1,2-diol # PATENT # Inhibitors of inducible form of 6-
phosphofructose-2-kinase # EP-2702043-A1
CHEMBL3730140 # 6-[4-chloro-3-(dimethylamino)phenyl]-2-methyl-N-[(1R)-1,2,3,4-tetrahydronaphthalen-1-yl]pyrimidin-
4-amine # PATENT # Inhibitors of inducible form of 6-phosphofructose-2-kinase # EP-2702043-A1
CHEMBL3730144 # 2-methyl-6-(3-methyl-1-benzofuran-5-yl)-N-{1-[3-(thiophen-3-yl)phenyl]ethyl}pyrimidin-4-amine #
PATENT # Inhibitors of inducible form of 6-phosphofructose-2-kinase # EP-2702043-A1
CHEMBL3730150 # N-[(1S)-1-[3-(5-fluoropyridin-3-yl)phenyl]ethyl]-2-methyl-6-(3-methyl-1-benzothiophen-5-yl)pyrimidin-
4-amine # PATENT # Inhibitors of inducible form of 6-phosphofructose-2-kinase # EP-2702043-A1
CHEMBL3730166 # 6-(1-benzofuran-5-yl)-N-(1-{3-[(1,3-dimethyl-1H-pyrazol-5-yl)methoxy]phenyl}ethyl)-2-
methylpyrimidin-4-amine # PATENT # Inhibitors of inducible form of 6-phosphofructose-2-kinase # EP-2702043-A1
CHEMBL3730190 # 2-methyl-6-(3-methyl-1-benzofuran-5-yl)-N-(1-{3-[(piperidin-3-yl)methoxy]phenyl}ethyl)pyrimidin-4-
amine # PATENT # Inhibitors of inducible form of 6-phosphofructose-2-kinase # EP-2702043-A1
CHEMBL3730214 # 6-(4-chloro-3-fluorophenyl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3730220 # 1-(5-{3-[(1S)-1-{[2-methyl-6-(3-methyl-1-benzofuran-5-yl)pyrimidin-4-yl]amino}ethyl]phenyl}pyridin-3-
yl)ethan-1-ol # PATENT # Inhibitors of inducible form of 6-phosphofructose-2-kinase # EP-2702043-A1
CHEMBL3730221 # 2-[3-(1-{[6-(1,3-benzothiazol-6-yl)-2-methylpyrimidin-4-yl]amino}ethyl)phenoxy]-N-[2-
(dimethylamino)ethyl]acetamide # PATENT # Inhibitors of inducible form of 6-phosphofructose-2-kinase # EP-2702043-
A1
CHEMBL3730238 # N-{1-[3-(2-aminopyrimidin-5-yl)phenyl]ethyl}-6-(1,3-benzothiazol-6-yl)-2-methylpyrimidin-4-amine #
PATENT # Inhibitors of inducible form of 6-phosphofructose-2-kinase # EP-2702043-A1
CHEMBL3730251 # 2-{2-[4-(5-{3-[(1S)-1-{[2-methyl-6-(3-methyl-1-benzofuran-5-yl)pyrimidin-4-
yl]amino}ethyl]phenyl}pyrimidin-2-yl)piperazin-1-yl]ethoxy}ethan-1-ol # PATENT # Inhibitors of inducible form of 6-
phosphofructose-2-kinase # EP-2702043-A1
CHEMBL3730278 # ethyl 2-(4-{3-[(1S)-1-{[6-(1,3-benzothiazol-6-yl)-2-methylpyrimidin-4-yl]amino}ethyl]phenyl}-1H-
pyrazol-1-yl)acetate # PATENT # Inhibitors of inducible form of 6-phosphofructose-2-kinase # EP-2702043-A1
CHEMBL3730310 # N-[(1S)-1-{3-[2-(diethylamino)pyrimidin-5-yl]phenyl]ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3730329 # N-[(1S)-1-[3-(6-chloro-5-methylpyridin-3-yl)phenyl]ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3730333 # (5-{3-[(1S)-1-{[2-methyl-6-(3-methyl-1-benzofuran-5-yl)pyrimidin-4-yl]amino}ethyl]phenyl}pyridin-3-
yl)methanol # PATENT # Inhibitors of inducible form of 6-phosphofructose-2-kinase # EP-2702043-A1
CHEMBL3730347 # 6-(3-ethyl-1-benzofuran-5-yl)-2-methyl-N-{1-[3-(1-methyl-1H-pyrazol-4-yl)phenyl]ethyl}pyrimidin-4-
amine # PATENT # Inhibitors of inducible form of 6-phosphofructose-2-kinase # EP-2702043-A1
CHEMBL3730380 # 3-(2-methyl-6-{[(1R)-1,2,3,4-tetrahydronaphthalen-1-yl]amino}pyrimidin-4-yl)benzonitrile # PATENT
# Inhibitors of inducible form of 6-phosphofructose-2-kinase # EP-2702043-A1
CHEMBL3730398 # 6-(1,3-benzothiazol-6-yl)-2-methyl-N-[(1S)-1-{3-[6-(morpholin-4-yl)pyridin-3-yl]phenyl}ethyl]pyrimidin-
4-amine # PATENT # Inhibitors of inducible form of 6-phosphofructose-2-kinase # EP-2702043-A1
CHEMBL3730429 # 6-(1,3-benzothiazol-6-yl)-N-{1-[3-(2-{[2-(dimethylamino)ethyl]amino}pyrimidin-5-yl)phenyl]ethyl}-2-
methylpyrimidin-4-amine # PATENT # Inhibitors of inducible form of 6-phosphofructose-2-kinase # EP-2702043-A1
CHEMBL3730444 # N-[(1S)-1-[3-(2-chloropyrimidin-5-yl)phenyl]ethyl]-2-methyl-6-(3-methyl-1-benzofuran-5-yl)pyrimidin-
4-amine # PATENT # Inhibitors of inducible form of 6-phosphofructose-2-kinase # EP-2702043-A1
CHEMBL3730452 # 6-(4-chloro-3-fluorophenyl)-2-methyl-N-{1-[5-(1-methyl-1H-pyrazol-4-yl)pyridin-3-yl]ethyl}pyrimidin-4-
amine # PATENT # Inhibitors of inducible form of 6-phosphofructose-2-kinase # EP-2702043-A1
CHEMBL3730460 # ethyl 5-[3-(1-{[6-(1,3-benzothiazol-6-yl)-2-methylpyrimidin-4-yl]amino}ethyl)phenyl]pyridine-3-
carboxylate # PATENT # Inhibitors of inducible form of 6-phosphofructose-2-kinase # EP-2702043-A1
CHEMBL3730462 # 6-(3-methoxy-4-methylphenyl)-2-methyl-N-[(1R)-1,2,3,4-tetrahydronaphthalen-1-yl]pyrimidin-4-amine
# PATENT # Inhibitors of inducible form of 6-phosphofructose-2-kinase # EP-2702043-A1
CHEMBL3730466 # (5-{3-[(1S)-1-{[6-(1,3-benzothiazol-6-yl)-2-methylpyrimidin-4-yl]amino}ethyl]phenyl}pyridin-2-
yl)methanol # PATENT # Inhibitors of inducible form of 6-phosphofructose-2-kinase # EP-2702043-A1
CHEMBL3730485 # 2-{4-[3-(1-{[6-(1,3-benzothiazol-6-yl)-2-methylpyrimidin-4-yl]amino}ethyl)phenyl]-1H-pyrazol-1-
yl}acetic acid # PATENT # Inhibitors of inducible form of 6-phosphofructose-2-kinase # EP-2702043-A1
CHEMBL3730496 # N-[(1S)-1-(3-bromophenyl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3730509 # 6-(1,3-benzothiazol-6-yl)-N-[(1S)-1-{3-[2-(ethylamino)pyrimidin-5-yl]phenyl}ethyl]-2-methylpyrimidin-
4-amine # PATENT # Inhibitors of inducible form of 6-phosphofructose-2-kinase # EP-2702043-A1
CHEMBL3730510 # 2-[3-(1-{[6-(1,3-benzothiazol-6-yl)-2-methylpyrimidin-4-yl]amino}ethyl)phenoxy]propanamide #
PATENT # Inhibitors of inducible form of 6-phosphofructose-2-kinase # EP-2702043-A1
CHEMBL3730529 # 2-{[2-chloro-5-(2-methyl-6-{[(1R)-1,2,3,4-tetrahydronaphthalen-1-yl]amino}pyrimidin-4-
yl)phenyl]amino}acetonitrile # PATENT # Inhibitors of inducible form of 6-phosphofructose-2-kinase # EP-2702043-A1
CHEMBL3730530 # 2-methyl-6-(4-methyl-3,4-dihydro-2H-1,4-benzoxazin-6-yl)-N-[(1R)-1,2,3,4-tetrahydronaphthalen-1-
yl]pyrimidin-4-amine # PATENT # Inhibitors of inducible form of 6-phosphofructose-2-kinase # EP-2702043-A1
CHEMBL3730532 # 1-[3-(1-{[6-(1,3-benzothiazol-6-yl)-2-methylpyrimidin-4-yl]amino}ethyl)phenoxy]propan-2-ol #
PATENT # Inhibitors of inducible form of 6-phosphofructose-2-kinase # EP-2702043-A1
CHEMBL3730536 # 6-(1-benzothiophen-5-yl)-N-[(4R)-3,4-dihydro-2H-1-benzopyran-4-yl]-2-methylpyrimidin-4-amine #
PATENT # Inhibitors of inducible form of 6-phosphofructose-2-kinase # EP-2702043-A1
CHEMBL3730547 # 2-methyl-6-[4-(methylamino)phenyl]-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3730549 # 6-(1,3-benzothiazol-6-yl)-N-[(1S)-1-[3-(6-chloro-5-methylpyridin-3-yl)phenyl]ethyl]-2-methylpyrimidin-
4-amine # PATENT # Inhibitors of inducible form of 6-phosphofructose-2-kinase # EP-2702043-A1
CHEMBL3730569 # N-[(1S)-1-[3-(5-aminopyridin-3-yl)phenyl]ethyl]-6-(4-chloro-3-fluorophenyl)-2-methylpyrimidin-4-
amine # PATENT # Inhibitors of inducible form of 6-phosphofructose-2-kinase # EP-2702043-A1
CHEMBL3730571 # ethyl 5-{3-[(1S)-1-{[2-methyl-6-(3-methyl-1-benzofuran-5-yl)pyrimidin-4-
yl]amino}ethyl]phenyl}pyridine-3-carboxylate # PATENT # Inhibitors of inducible form of 6-phosphofructose-2-kinase #
EP-2702043-A1
CHEMBL3730573 # 6-(1-benzothiophen-5-yl)-2-methyl-N-{1-[3-(1-methyl-1H-pyrazol-4-yl)phenyl]ethyl}pyrimidin-4-amine
# PATENT # Inhibitors of inducible form of 6-phosphofructose-2-kinase # EP-2702043-A1
CHEMBL3730575 # N-ethyl-2-[3-(1-{[2-methyl-6-(1-methyl-1H-indol-6-yl)pyrimidin-4-yl]amino}ethyl)phenoxy]acetamide #
PATENT # Inhibitors of inducible form of 6-phosphofructose-2-kinase # EP-2702043-A1
CHEMBL3730587 # 6-(1-benzofuran-5-yl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3730588 # 6-(1H-indol-5-yl)-2-methyl-N-[(1R)-1,2,3,4-tetrahydronaphthalen-1-yl]pyrimidin-4-amine # PATENT #
Inhibitors of inducible form of 6-phosphofructose-2-kinase # EP-2702043-A1
CHEMBL3730589 # 5-{3-[(1S)-1-{[2-methyl-6-(3-methyl-1-benzofuran-5-yl)pyrimidin-4-yl]amino}ethyl]phenyl}pyridine-3-
carboxamide # PATENT # Inhibitors of inducible form of 6-phosphofructose-2-kinase # EP-2702043-A1
CHEMBL3730603 # 3-[3-(1-{[6-(1,3-benzothiazol-6-yl)-2-methylpyrimidin-4-yl]amino}ethyl)phenoxy]propan-1-ol #
PATENT # Inhibitors of inducible form of 6-phosphofructose-2-kinase # EP-2702043-A1
CHEMBL3730614 # 2-methyl-6-(3-methyl-1-benzofuran-5-yl)-N-[(1S)-1-[3-(2-{[(oxolan-2-yl)methyl]amino}pyrimidin-5-
yl)phenyl]ethyl]pyrimidin-4-amine # PATENT # Inhibitors of inducible form of 6-phosphofructose-2-kinase # EP-2702043-
A1
CHEMBL3730672 # N-(1-{3-[(1,3-dimethyl-1H-pyrazol-5-yl)methoxy]phenyl}ethyl)-2-methyl-6-(1-methyl-1H-indol-6-
yl)pyrimidin-4-amine # PATENT # Inhibitors of inducible form of 6-phosphofructose-2-kinase # EP-2702043-A1
CHEMBL3730680 # 6-(1,3-benzothiazol-6-yl)-2-methyl-N-{1-[3-(1H-1,2,3,4-tetrazol-1-yl)phenyl]ethyl}pyrimidin-4-amine #
PATENT # Inhibitors of inducible form of 6-phosphofructose-2-kinase # EP-2702043-A1
CHEMBL3730684 # 6-(1,3-benzothiazol-6-yl)-N-(3,4-dihydro-1H-2-benzothiopyran-4-yl)-2-methylpyrimidin-4-amine #
PATENT # Inhibitors of inducible form of 6-phosphofructose-2-kinase # EP-2702043-A1
CHEMBL3730686 # N-(1-{3-[2-(1H-imidazol-1-yl)ethoxy]phenyl}ethyl)-2-methyl-6-(3-methyl-1-benzofuran-5-yl)pyrimidin-
4-amine # PATENT # Inhibitors of inducible form of 6-phosphofructose-2-kinase # EP-2702043-A1
CHEMBL3730693 # 2-methyl-N-{1-[3-(1-methyl-1H-pyrazol-4-yl)phenyl]ethyl}-6-(3,4,5-trifluorophenyl)pyrimidin-4-amine #
PATENT # Inhibitors of inducible form of 6-phosphofructose-2-kinase # EP-2702043-A1
CHEMBL3730718 # 1-(5-{3-[(1S)-1-{[6-(1,3-benzothiazol-6-yl)-2-methylpyrimidin-4-yl]amino}ethyl]phenyl}pyrimidin-2-
yl)piperidin-4-ol # PATENT # Inhibitors of inducible form of 6-phosphofructose-2-kinase # EP-2702043-A1
CHEMBL3730721 # N-[(1S)-1-[3-(5-aminopyridin-3-yl)phenyl]ethyl]-6-(1,3-benzothiazol-6-yl)-2-methylpyrimidin-4-amine #
PATENT # Inhibitors of inducible form of 6-phosphofructose-2-kinase # EP-2702043-A1
CHEMBL3730726 # N-[1-(2-fluoro-3-methoxyphenyl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3730737 # 6-(1,3-benzothiazol-6-yl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3730750 # 6-(1,3-benzothiazol-6-yl)-N-[(1-cyclopentanecarbonylpiperidin-3-yl)methyl]-2-methylpyrimidin-4-
amine # PATENT # Inhibitors of inducible form of 6-phosphofructose-2-kinase # EP-2702043-A1
CHEMBL3730762 # 1-[4-(5-{3-[(1S)-1-{[2-methyl-6-(3-methyl-1-benzothiophen-5-yl)pyrimidin-4-
yl]amino}ethyl]phenyl}pyrimidin-2-yl)piperazin-1-yl]ethan-1-one # PATENT # Inhibitors of inducible form of 6-
phosphofructose-2-kinase # EP-2702043-A1
CHEMBL3730773 # N-[1-(2-fluorophenyl)ethyl]-2-methyl-6-(3-methyl-1-benzofuran-5-yl)pyrimidin-4-amine # PATENT #
Inhibitors of inducible form of 6-phosphofructose-2-kinase # EP-2702043-A1
CHEMBL3730783 # 6-(1,3-benzothiazol-6-yl)-2-methyl-N-(1-{3-[(3-methyl-1,2-oxazol-5-
yl)methoxy]phenyl}ethyl)pyrimidin-4-amine # PATENT # Inhibitors of inducible form of 6-phosphofructose-2-kinase # EP-
2702043-A1
CHEMBL3730788 # 2-chloro-5-(1-{[2-methyl-6-(3-methyl-1-benzofuran-5-yl)pyrimidin-4-yl]amino}ethyl)phenol # PATENT
# Inhibitors of inducible form of 6-phosphofructose-2-kinase # EP-2702043-A1
CHEMBL3730814 # 2-[3-(1-{[6-(1,3-benzothiazol-6-yl)-2-methylpyrimidin-4-yl]amino}ethyl)phenoxy]-N,N-
diethylpropanamide # PATENT # Inhibitors of inducible form of 6-phosphofructose-2-kinase # EP-2702043-A1
CHEMBL3730824 # N-[(1S)-1-[3-(2-{[(furan-2-yl)methyl]amino}pyrimidin-5-yl)phenyl]ethyl]-2-methyl-6-(3-methyl-1-
benzofuran-5-yl)pyrimidin-4-amine # PATENT # Inhibitors of inducible form of 6-phosphofructose-2-kinase # EP-
2702043-A1
CHEMBL3730833 # 2-[3-(1-{[2-methyl-6-(1-methyl-1H-indol-2-yl)pyrimidin-4-yl]amino}ethyl)phenoxy]acetamide #
PATENT # Inhibitors of inducible form of 6-phosphofructose-2-kinase # EP-2702043-A1
CHEMBL3730840 # 2-[3-({[6-(1,3-benzothiazol-6-yl)-2-methylpyrimidin-4-yl]amino}methyl)-4-fluorophenoxy]-1-
(morpholin-4-yl)ethan-1-one # PATENT # Inhibitors of inducible form of 6-phosphofructose-2-kinase # EP-2702043-A1
CHEMBL3730841 # 6-(2-fluoro-3-methoxyphenyl)-N-[(1S)-1-(3-methoxyphenyl)ethyl]-2-methylpyrimidin-4-amine #
PATENT # Inhibitors of inducible form of 6-phosphofructose-2-kinase # EP-2702043-A1
CHEMBL3730857 # 6-(1,3-benzothiazol-6-yl)-N-{1-[3-(2-fluoroethoxy)phenyl]ethyl}-2-methylpyrimidin-4-amine # PATENT
# Inhibitors of inducible form of 6-phosphofructose-2-kinase # EP-2702043-A1
CHEMBL3730861 # 6-(1,3-benzothiazol-6-yl)-2-methyl-N-[(1S)-1-{3-[2-(4-methylpiperazin-1-yl)pyrimidin-5-
yl]phenyl}ethyl]pyrimidin-4-amine # PATENT # Inhibitors of inducible form of 6-phosphofructose-2-kinase # EP-2702043-
A1
CHEMBL3730870 # 6-(1,3-benzothiazol-6-yl)-2-methyl-N-{1-[3-(2-methylpyridin-4-yl)phenyl]ethyl}pyrimidin-4-amine #
PATENT # Inhibitors of inducible form of 6-phosphofructose-2-kinase # EP-2702043-A1
CHEMBL3730877 # 3-[3-(1-{[6-(1,3-benzothiazol-6-yl)-2-methylpyrimidin-4-yl]amino}ethyl)phenoxy]propane-1,2-diol #
PATENT # Inhibitors of inducible form of 6-phosphofructose-2-kinase # EP-2702043-A1
CHEMBL3730880 # 6-(4-fluoro-3-methylphenyl)-2-methyl-N-{1-[3-(1-methyl-1H-pyrazol-4-yl)phenyl]ethyl}pyrimidin-4-
amine # PATENT # Inhibitors of inducible form of 6-phosphofructose-2-kinase # EP-2702043-A1
CHEMBL3730887 # N-[(1S)-1-(3-methoxyphenyl)ethyl]-2-methyl-6-(3-methylphenyl)pyrimidin-4-amine # PATENT #
Inhibitors of inducible form of 6-phosphofructose-2-kinase # EP-2702043-A1
CHEMBL3730900 # N-[(1S)-1-[3-(6-fluoro-5-methylpyridin-3-yl)phenyl]ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3730918 # 2-[3-(1-{[2-methyl-6-(3-methyl-1-benzofuran-5-yl)pyrimidin-4-yl]amino}ethyl)phenoxy]-1-(morpholin-
4-yl)ethan-1-one # PATENT # Inhibitors of inducible form of 6-phosphofructose-2-kinase # EP-2702043-A1
CHEMBL3730949 # 2-methyl-6-(naphthalen-2-yl)-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3730954 # 6-(3-chlorophenyl)-N-[(1S)-1-(3-methoxyphenyl)ethyl]-2-methylpyrimidin-4-amine # PATENT #
Inhibitors of inducible form of 6-phosphofructose-2-kinase # EP-2702043-A1
CHEMBL3730964 # N-[(1S)-1-{6′-fluoro-[3,3′-bipyridin]-5-yl}ethyl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3730971 # N-[1-(5-bromopyridin-3-yl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3730973 # 4-[3-(1-{[6-(1,3-benzothiazol-6-yl)-2-methylpyrimidin-4-yl]amino}ethyl)phenoxy]-N,N-
dimethylbutanamide # PATENT # Inhibitors of inducible form of 6-phosphofructose-2-kinase # EP-2702043-A1
CHEMBL3731006 # N-[(1R)-1-(3-methoxyphenyl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3731016 # 1-(5-{3-[(1S)-1-{[2-methyl-6-(3-methyl-1-benzofuran-5-yl)pyrimidin-4-yl]amino}ethyl]phenyl}pyrimidin-
2-yl)pyrrolidin-3-ol # PATENT # Inhibitors of inducible form of 6-phosphofructose-2-kinase # EP-2702043-A1
CHEMBL3731033 # 3-(1-{[6-(1,3-benzothiazol-6-yl)-2-methylpyrimidin-4-yl]amino}ethyl)-N-(but-2-yn-1-yl)benzene-1-
sulfonamide # PATENT # Inhibitors of inducible form of 6-phosphofructose-2-kinase # EP-2702043-A1
CHEMBL3731046 # 3-(1-{[2-methyl-6-(3-methyl-1-benzofuran-5-yl)pyrimidin-4-yl]amino}ethyl)phenol # PATENT #
Inhibitors of inducible form of 6-phosphofructose-2-kinase # EP-2702043-A1
CHEMBL3731052 # 2-[3-(1-{[6-(1,3-benzothiazol-6-yl)-2-methylpyrimidin-4-yl]amino}ethyl)phenoxy]ethan-1-ol # PATENT
# Inhibitors of inducible form of 6-phosphofructose-2-kinase # EP-2702043-A1
CHEMBL3731061 # 2-[3-(1-{[6-(3-methoxyphenyl)-2-methylpyrimidin-4-yl]amino}ethyl)phenoxy]acetonitrile # PATENT #
Inhibitors of inducible form of 6-phosphofructose-2-kinase # EP-2702043-A1
CHEMBL3731069 # 6-(2,4-dichlorophenyl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3731070 # 6-(3-methoxyphenyl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3731074 # N-[1-(3-aminophenyl)ethyl]-2-methyl-6-(3-methyl-1-benzofuran-5-yl)pyrimidin-4-amine # PATENT #
Inhibitors of inducible form of 6-phosphofructose-2-kinase # EP-2702043-A1
CHEMBL3731084 # 2-methyl-6-(3-methyl-1-benzofuran-5-yl)-N-[1-(3-methylphenyl)ethyl]pyrimidin-4-amine # PATENT #
Inhibitors of inducible form of 6-phosphofructose-2-kinase # EP-2702043-A1
CHEMBL3731121 # N-[(1S)-1-[3-(6-fluoropyridin-3-yl)phenyl]ethyl]-2-methyl-6-(3-methyl-1-benzothiophen-5-yl)pyrimidin-
4-amine # PATENT # Inhibitors of inducible form of 6-phosphofructose-2-kinase # EP-2702043-A1
CHEMBL3731123 # 6-(1,3-benzothiazol-6-yl)-2-methyl-N-(3-methylbutyl)pyrimidin-4-amine # PATENT # Inhibitors of
inducible form of 6-phosphofructose-2-kinase # EP-2702043-A1
CHEMBL3731150 # 2-methyl-N-[(1R)-1,2,3,4-tetrahydronaphthalen-1-yl]-6-[4-(trifluoromethyl)phenyl]pyrimidin-4-amine #
PATENT # Inhibitors of inducible form of 6-phosphofructose-2-kinase # EP-2702043-A1
CHEMBL3731164 # 6-(3,4-dichlorophenyl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3731195 # 1-(5-{3-[(1S)-1-{[2-methyl-6-(3-methyl-1-benzofuran-5-yl)pyrimidin-4-yl]amino}ethyl]phenyl}pyridin-2-
yl)piperidin-4-ol # PATENT # Inhibitors of inducible form of 6-phosphofructose-2-kinase # EP-2702043-A1
CHEMBL3731212 # 6-(1,3-benzothiazol-6-yl)-N-[(1S)-1-[3-(2-{[3-(dimethylamino)propyl]amino}pyrimidin-5-
yl)phenyl]ethyl]-2-methylpyrimidin-4-amine # PATENT # Inhibitors of inducible form of 6-phosphofructose-2-kinase # EP-
2702043-A1
CHEMBL3731213 # 2-methyl-6-(3-methyl-1-benzofuran-5-yl)-N-{1-[3-(4H-1,2,4-triazol-4-yl)phenyl]ethyl}pyrimidin-4-
amine # PATENT # Inhibitors of inducible form of 6-phosphofructose-2-kinase # EP-2702043-A1
CHEMBL3731228 # 2-methyl-6-(3-methyl-1-benzofuran-5-yl)-N-[(1S)-1-[3-(1-methyl-1H-pyrrol-2-yl)phenyl]ethyl]pyrimidin-
4-amine # PATENT # Inhibitors of inducible form of 6-phosphofructose-2-kinase # EP-2702043-A1
CHEMBL3731229 # 2-methyl-6-(3-methyl-1-benzofuran-5-yl)-N-[(1S)-1-phenylethyl]pyrimidin-4-amine # PATENT #
Inhibitors of inducible form of 6-phosphofructose-2-kinase # EP-2702043-A1
CHEMBL3731234 # N-[(1S)-1-{5′-fluoro-[3,3′-bipyridin]-5-yl}ethyl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3731256 # 6-[4-chloro-3-(methylamino)phenyl]-2-methyl-N-[(1R)-1,2,3,4-tetrahydronaphthalen-1-yl]pyrimidin-4-
amine # PATENT # Inhibitors of inducible form of 6-phosphofructose-2-kinase # EP-2702043-A1
CHEMBL3731258 # 2-methyl-6-(3-methyl-1-benzofuran-5-yl)-N-{1-[4-methyl-3-(1-methyl-1H-pyrazol-4-
yl)phenyl]ethyl}pyrimidin-4-amine # PATENT # Inhibitors of inducible form of 6-phosphofructose-2-kinase # EP-2702043-
A1
CHEMBL3731265 # 3-(1-{[6-(1,3-benzothiazol-6-yl)-2-methylpyrimidin-4-yl]amino}ethyl)-N-(2-hydroxyethyl)benzene-1-
sulfonamide # PATENT # Inhibitors of inducible form of 6-phosphofructose-2-kinase # EP-2702043-A1
CHEMBL3731279 # N-(1-{3-[3-(dimethylamino)propoxy]phenyl}ethyl)-2-methyl-6-(3-methyl-1-benzofuran-5-yl)pyrimidin-
4-amine # PATENT # Inhibitors of inducible form of 6-phosphofructose-2-kinase # EP-2702043-A1
CHEMBL3731294 # 6-(1,3-benzothiazol-6-yl)-2-methyl-N-{1-[3-(morpholin-4-yl)phenyl]ethyl}pyrimidin-4-amine # PATENT
# Inhibitors of inducible form of 6-phosphofructose-2-kinase # EP-2702043-A1
CHEMBL3731328 # 6-(1,3-benzothiazol-6-yl)-N-(3,4-dihydro-2H-1-benzothiopyran-4-yl)-2-methylpyrimidin-4-amine #
PATENT # Inhibitors of inducible form of 6-phosphofructose-2-kinase # EP-2702043-A1
CHEMBL3731331 # N-(1-{3-[(1,3-dimethyl-1H-pyrazol-5-yl)methoxy]phenyl}ethyl)-2-methyl-6-(1-methyl-1H-indol-2-
yl)pyrimidin-4-amine # PATENT # Inhibitors of inducible form of 6-phosphofructose-2-kinase # EP-2702043-A1
CHEMBL3731349 # 1-(4-{2-[3-(1-{[6-(1,3-benzothiazol-6-yl)-2-methylpyrimidin-4-yl]amino}ethyl)phenoxy]ethyl}piperazin-
1-yl)ethan-1-one # PATENT # Inhibitors of inducible form of 6-phosphofructose-2-kinase # EP-2702043-A1
CHEMBL3731362 # 3-[(5-{3-[(1S)-1-{[2-methyl-6-(3-methyl-1-benzofuran-5-yl)pyrimidin-4-
yl]amino}ethyl]phenyl}pyrimidin-2-yl)amino]propan-1-ol # PATENT # Inhibitors of inducible form of 6-phosphofructose-2-
kinase # EP-2702043-A1
CHEMBL3731364 # 6-(4-chloro-3-fluorophenyl)-2-methyl-N-[(1S)-1-{3-[6-(piperazin-1-yl)pyridin-3-
yl]phenyl}ethyl]pyrimidin-4-amine # PATENT # Inhibitors of inducible form of 6-phosphofructose-2-kinase # EP-2702043-
A1
CHEMBL3731371 # 2-methyl-6-(3-methyl-1-benzofuran-5-yl)-N-[(1S)-1-{3-[6-(methylamino)pyridin-3-
yl]phenyl}ethyl]pyrimidin-4-amine # PATENT # Inhibitors of inducible form of 6-phosphofructose-2-kinase # EP-2702043-
A1
CHEMBL3731372 # 6-(1,3-benzothiazol-6-yl)-2-methyl-N-(1-{3-[2-(1H-pyrrol-1-yl)ethoxy]phenyl}ethyl)pyrimidin-4-amine #
PATENT # Inhibitors of inducible form of 6-phosphofructose-2-kinase # EP-2702043-A1
CHEMBL3731388 # 2-methyl-6-(3-methylphenyl)-N-[(1R)-1,2,3,4-tetrahydronaphthalen-1-yl]pyrimidin-4-amine # PATENT
# Inhibitors of inducible form of 6-phosphofructose-2-kinase # EP-2702043-A1
CHEMBL3731392 # 1-(5-{5-[(1S)-1-{[2-methyl-6-(3-methyl-1-benzofuran-5-yl)pyrimidin-4-yl]amino}ethyl]pyridin-3-
yl}pyrimidin-2-yl)piperidin-4-ol # PATENT # Inhibitors of inducible form of 6-phosphofructose-2-kinase # EP-2702043-A1
CHEMBL3731398 # 2-[3-({[6-(1,3-benzothiazol-6-yl)-2-methylpyrimidin-4-yl]amino}methyl)-4-fluorophenoxy]-N,N-
diethylacetamide # PATENT # Inhibitors of inducible form of 6-phosphofructose-2-kinase # EP-2702043-A1
CHEMBL3731409 # N,N-dimethyl-2-{3-[(1S)-1-{[2-methyl-6-(3-methyl-1-benzofuran-5-yl)pyrimidin-4-
yl]amino}ethyl]phenoxy}acetamide # PATENT # Inhibitors of inducible form of 6-phosphofructose-2-kinase # EP-
2702043-A1
CHEMBL3731413 # 2-methyl-6-(3-methyl-1-benzofuran-5-yl)-N-{1-[3-(1H-1,2,3,4-tetrazol-1-yl)phenyl]ethyl}pyrimidin-4-
amine # PATENT # Inhibitors of inducible form of 6-phosphofructose-2-kinase # EP-2702043-A1
CHEMBL3731414 # 2-[3-(1-{[6-(1,3-benzothiazol-6-yl)-2-methylpyrimidin-4-yl]amino}ethyl)phenoxy]-1-(2-methylaziridin-1-
yl)ethan-1-one # PATENT # Inhibitors of inducible form of 6-phosphofructose-2-kinase # EP-2702043-A1
CHEMBL3731440 # [2-chloro-5-(2-methyl-6-{[(1R)-1,2,3,4-tetrahydronaphthalen-1-yl]amino}pyrimidin-4-
yl)phenyl]methanol # PATENT # Inhibitors of inducible form of 6-phosphofructose-2-kinase # EP-2702043-A1
CHEMBL3731463 # 6-(1,3-benzothiazol-6-yl)-N-(6-methoxy-1,2,3,4-tetrahydronaphthalen-1-yl)-2-methylpyrimidin-4-
amine # PATENT # Inhibitors of inducible form of 6-phosphofructose-2-kinase # EP-2702043-A1
CHEMBL3731466 # 2-methyl-6-(4-methylphenyl)-N-[(1R)-1,2,3,4-tetrahydronaphthalen-1-yl]pyrimidin-4-amine # PATENT
# Inhibitors of inducible form of 6-phosphofructose-2-kinase # EP-2702043-A1
CHEMBL3731473 # 6-(3-fluorophenyl)-2-methyl-N-[(1R)-1,2,3,4-tetrahydronaphthalen-1-yl]pyrimidin-4-amine # PATENT
# Inhibitors of inducible form of 6-phosphofructose-2-kinase # EP-2702043-A1
CHEMBL3731486 # 6-(1,3-benzothiazol-6-yl)-N-[(1S)-1-[3-(2-fluoropyridin-4-yl)phenyl]ethyl]-2-methylpyrimidin-4-amine #
PATENT # Inhibitors of inducible form of 6-phosphofructose-2-kinase # EP-2702043-A1
CHEMBL3731501 # 2-methyl-5-(2-methyl-6-{[(1R)-1,2,3,4-tetrahydronaphthalen-1-yl]amino}pyrimidin-4-yl)phenol #
PATENT # Inhibitors of inducible form of 6-phosphofructose-2-kinase # EP-2702043-A1
CHEMBL3731514 # 6-(1-benzothiophen-5-yl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3731527 # 6-(2,3-dihydro-1-benzofuran-5-yl)-2-methyl-N-{1-[3-(1-methyl-1H-pyrazol-4-yl)phenyl]ethyl}pyrimidin-
4-amine # PATENT # Inhibitors of inducible form of 6-phosphofructose-2-kinase # EP-2702043-A1
CHEMBL3731531 # 3-(1-{[2-methyl-6-(1-methyl-1H-indol-6-yl)pyrimidin-4-yl]amino}ethyl)benzene-1-sulfonamide #
PATENT # Inhibitors of inducible form of 6-phosphofructose-2-kinase # EP-2702043-A1
CHEMBL3731532 # N-[(1S)-1-{3-[6-(4-ethylpiperazin-1-yl)pyridin-3-yl]phenyl}ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3731534 # N,N-dimethyl-2-[3-(1-{[2-methyl-6-(3-methyl-1-benzofuran-5-yl)pyrimidin-4-
yl]amino}ethyl)phenoxy]acetamide # PATENT # Inhibitors of inducible form of 6-phosphofructose-2-kinase # EP-
2702043-A1
CHEMBL3731539 # 2-methyl-6-(3-methyl-1-benzofuran-5-yl)-N-[(1S)-1-[3-(2H-1,2,3-triazol-2-yl)phenyl]ethyl]pyrimidin-4-
amine # PATENT # Inhibitors of inducible form of 6-phosphofructose-2-kinase # EP-2702043-A1
CHEMBL3731555 # 6-(1,3-benzothiazol-6-yl)-2-methyl-N-[1-(pyridin-3-yl)ethyl]pyrimidin-4-amine # PATENT # Inhibitors
of inducible form of 6-phosphofructose-2-kinase # EP-2702043-A1
CHEMBL3731563 # N-[(1S)-1-{3-[2-(ethylamino)pyrimidin-5-yl]phenyl}ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3731571 # 2-[3-(1-{[6-(1,3-benzothiazol-6-yl)-2-methylpyrimidin-4-yl]amino}ethyl)phenoxy]-N,N-
dimethylacetamide # PATENT # Inhibitors of inducible form of 6-phosphofructose-2-kinase # EP-2702043-A1
CHEMBL3731574 # methyl 2-chloro-5-(2-methyl-6-{[(1R)-1,2,3,4-tetrahydronaphthalen-1-yl]amino}pyrimidin-4-
yl)benzoate # PATENT # Inhibitors of inducible form of 6-phosphofructose-2-kinase # EP-2702043-A1
CHEMBL3731578 # N-(5-{[6-(1,3-benzothiazol-6-yl)-2-methylpyrimidin-4-yl]amino}-5,6,7,8-tetrahydronaphthalen-2-
yl)propanamide # PATENT # Inhibitors of inducible form of 6-phosphofructose-2-kinase # EP-2702043-A1
CHEMBL3731580 # 6-(4-chloro-3-fluorophenyl)-2-methyl-N-{1-[3-(1-methyl-1H-pyrazol-4-yl)phenyl]ethyl}pyrimidin-4-
amine # PATENT # Inhibitors of inducible form of 6-phosphofructose-2-kinase # EP-2702043-A1
CHEMBL3731585 # 2-[3-(1-{[6-(3-methoxy-4-methylphenyl)-2-methylpyrimidin-4-yl]amino}ethyl)phenoxy]acetamide #
PATENT # Inhibitors of inducible form of 6-phosphofructose-2-kinase # EP-2702043-A1
CHEMBL3731588 # 2-[3-(1-{[6-(1,3-benzothiazol-6-yl)-2-methylpyrimidin-4-yl]amino}ethyl)phenoxy]-N-(2-
hydroxyethyl)acetamide # PATENT # Inhibitors of inducible form of 6-phosphofructose-2-kinase # EP-2702043-A1
CHEMBL3731592 # 2-{[2-methyl-5-(2-methyl-6-{[(1R)-1,2,3,4-tetrahydronaphthalen-1-yl]amino}pyrimidin-4-
yl)phenyl]amino}acetonitrile # PATENT # Inhibitors of inducible form of 6-phosphofructose-2-kinase # EP-2702043-A1
CHEMBL3731613 # N-[(1S)-1-[3-(6-chloropyridin-3-yl)phenyl]ethyl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3731615 # N-[3-(1-{[2-methyl-6-(3-methyl-1-benzofuran-5-yl)pyrimidin-4-yl]amino}ethyl)phenyl]prop-2-enamide
# PATENT # Inhibitors of inducible form of 6-phosphofructose-2-kinase # EP-2702043-A1
CHEMBL3731619 # N,N-dimethyl-2-[3-(1-{[2-methyl-6-(1-methyl-1H-indol-6-yl)pyrimidin-4-
yl]amino}ethyl)phenoxy]acetamide # PATENT # Inhibitors of inducible form of 6-phosphofructose-2-kinase # EP-
2702043-A1
CHEMBL3731623 # N-[(1S)-1-[3-(2-{4-[2-(dimethylamino)ethyl]piperazin-1-yl}pyrimidin-5-yl)phenyl]ethyl]-2-methyl-6-(3-
methyl-1-benzothiophen-5-yl)pyrimidin-4-amine # PATENT # Inhibitors of inducible form of 6-phosphofructose-2-kinase #
EP-2702043-A1
CHEMBL3731652 # 4-[3-(1-{[6-(1,3-benzothiazol-6-yl)-2-methylpyrimidin-4-yl]amino}ethyl)phenoxy]butanoic acid #
PATENT # Inhibitors of inducible form of 6-phosphofructose-2-kinase # EP-2702043-A1
CHEMBL3731658 # 2-[3-(1-{[2-methyl-6-(1-methyl-1H-indol-6-yl)pyrimidin-4-yl]amino}ethyl)phenoxy]acetic acid #
PATENT # Inhibitors of inducible form of 6-phosphofructose-2-kinase # EP-2702043-A1
CHEMBL3731662 # 6-(1,3-benzothiazol-6-yl)-N-(1-{3-[6-(dimethylamino)pyridin-3-yl]phenyl}ethyl)-2-methylpyrimidin-4-
amine # PATENT # Inhibitors of inducible form of 6-phosphofructose-2-kinase # EP-2702043-A1
CHEMBL3731665 # 2-methyl-6-(3-methyl-1-benzofuran-5-yl)-N-[(1S)-1-{3-[6-(4-methylpiperazin-1-yl)pyridin-3-
yl]phenyl}ethyl]pyrimidin-4-amine # PATENT # Inhibitors of inducible form of 6-phosphofructose-2-kinase # EP-2702043-
A1
CHEMBL3731678 # ethyl 2-{2-[3-(1-{[2-methyl-6-(1-methyl-1H-indol-6-yl)pyrimidin-4-
yl]amino}ethyl)phenoxy]acetamido}acetate # PATENT # Inhibitors of inducible form of 6-phosphofructose-2-kinase # EP-
2702043-A1
CHEMBL3731685 # 6-(1,3-benzothiazol-6-yl)-2-methyl-N-[(1S)-1-[3-(1-methyl-1H-pyrazol-5-yl)phenyl]ethyl]pyrimidin-4-
amine # PATENT # Inhibitors of inducible form of 6-phosphofructose-2-kinase # EP-2702043-A1
CHEMBL3731698 # 6-(3-fluorophenyl)-2-methyl-N-[(1S)-1-[3-(1-methyl-1H-pyrazol-4-yl)phenyl]ethyl]pyrimidin-4-amine #
PATENT # Inhibitors of inducible form of 6-phosphofructose-2-kinase # EP-2702043-A1
CHEMBL3731706 # 2-methyl-N-{1-[3-(1-methyl-1H-pyrazol-4-yl)phenyl]ethyl}-6-(3-methylphenyl)pyrimidin-4-amine #
PATENT # Inhibitors of inducible form of 6-phosphofructose-2-kinase # EP-2702043-A1
CHEMBL3731708 # N-[(1S)-1-(3-methoxyphenyl)ethyl]-2-methyl-6-phenylpyrimidin-4-amine # PATENT # Inhibitors of
inducible form of 6-phosphofructose-2-kinase # EP-2702043-A1
CHEMBL3731723 # 2-methyl-6-(3-methyl-1-benzofuran-5-yl)-N-[(1S)-1-[3-(2-{4-[(1-methyl-1H-imidazol-2-
yl)methyl]piperazin-1-yl}pyrimidin-5-yl)phenyl]ethyl]pyrimidin-4-amine # PATENT # Inhibitors of inducible form of 6-
phosphofructose-2-kinase # EP-2702043-A1
CHEMBL3731734 # N-{1-[5-(2-aminopyrimidin-5-yl)pyridin-3-yl]ethyl}-6-(4-chloro-3-fluorophenyl)-2-methylpyrimidin-4-
amine # PATENT # Inhibitors of inducible form of 6-phosphofructose-2-kinase # EP-2702043-A1
CHEMBL3731743 # 2-methyl-6-(3-methyl-1-benzofuran-5-yl)-N-[(1S)-1-[3-(1-methyl-1H-pyrazol-5-
yl)phenyl]ethyl]pyrimidin-4-amine # PATENT # Inhibitors of inducible form of 6-phosphofructose-2-kinase # EP-2702043-
A1
CHEMBL3731774 # 6-(1,3-benzothiazol-6-yl)-2-methyl-N-{1-[3-(5-methyl-1H-1,2,3,4-tetrazol-1-yl)phenyl]ethyl}pyrimidin-
4-amine # PATENT # Inhibitors of inducible form of 6-phosphofructose-2-kinase # EP-2702043-A1
CHEMBL3731786 # 6-(1,3-benzothiazol-6-yl)-N-[(1S)-1-[3-(2-chloropyrimidin-5-yl)phenyl]ethyl]-2-methylpyrimidin-4-
amine # PATENT # Inhibitors of inducible form of 6-phosphofructose-2-kinase # EP-2702043-A1
CHEMBL3731800 # 2-methyl-1-[3-(1-{[2-methyl-6-(3-methyl-1-benzofuran-5-yl)pyrimidin-4-
yl]amino}ethyl)phenoxy]propan-2-ol # PATENT # Inhibitors of inducible form of 6-phosphofructose-2-kinase # EP-
2702043-A1
CHEMBL3731828 # 2-methyl-6-(3-methyl-1-benzofuran-5-yl)-N-[(1R)-1-[3-(1H-1,2,3,4-tetrazol-1-
yl)phenyl]ethyl]pyrimidin-4-amine # PATENT # Inhibitors of inducible form of 6-phosphofructose-2-kinase # EP-2702043-
A1
CHEMBL3731837 # 1-(5-{3-[(1S)-1-{[2-methyl-6-(3-methyl-1-benzofuran-5-yl)pyrimidin-4-yl]amino}ethyl]phenyl}pyridin-3-
yl)ethan-1-one # PATENT # Inhibitors of inducible form of 6-phosphofructose-2-kinase # EP-2702043-A1
CHEMBL3731868 # 6-(1,3-benzothiazol-6-yl)-2-methyl-N-(1-{3-[(piperidin-4-yl)methoxy]phenyl}ethyl)pyrimidin-4-amine #
PATENT # Inhibitors of inducible form of 6-phosphofructose-2-kinase # EP-2702043-A1
CHEMBL3731873 # 6-(7-fluoro-3-methyl-1-benzofuran-5-yl)-2-methyl-N-{1-[5-(1-methyl-1H-pyrazol-4-yl)pyridin-3-
yl]ethyl}pyrimidin-4-amine # PATENT # Inhibitors of inducible form of 6-phosphofructose-2-kinase # EP-2702043-A1
CHEMBL3731875 # N-[(1S)-1-[3-(6-aminopyridin-3-yl)phenyl]ethyl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3731886 # 6-(3-methoxy-4-methylphenyl)-N-[(1S)-1-(3-methoxyphenyl)ethyl]-2-methylpyrimidin-4-amine #
PATENT # Inhibitors of inducible form of 6-phosphofructose-2-kinase # EP-2702043-A1
CHEMBL3731890 # 2-[3-({[6-(1,3-benzothiazol-6-yl)-2-methylpyrimidin-4-yl]amino}methyl)-4-fluorophenoxy]-N,N-
dimethylacetamide # PATENT # Inhibitors of inducible form of 6-phosphofructose-2-kinase # EP-2702043-A1
CHEMBL3731911 # 2-[3-(1-{[6-(1,3-benzothiazol-6-yl)-2-methylpyrimidin-4-yl]amino}ethyl)phenoxy]-1-(morpholin-4-
yl)ethan-1-one # PATENT # Inhibitors of inducible form of 6-phosphofructose-2-kinase # EP-2702043-A1
CHEMBL3731927 # 6-(1,3-benzothiazol-6-yl)-2-methyl-N-[(2R)-2-phenylpropyl]pyrimidin-4-amine # PATENT # Inhibitors
of inducible form of 6-phosphofructose-2-kinase # EP-2702043-A1
CHEMBL3731945 # 3-(2-methyl-6-{[(1R)-1,2,3,4-tetrahydronaphthalen-1-yl]amino}pyrimidin-4-yl)phenol # PATENT #
Inhibitors of inducible form of 6-phosphofructose-2-kinase # EP-2702043-A1
CHEMBL3731988 # 2-(4-{3-[(1S)-1-{[2-methyl-6-(3-methyl-1-benzofuran-5-yl)pyrimidin-4-yl]amino}ethyl]phenyl}-1H-
pyrazol-1-yl)acetic acid # PATENT # Inhibitors of inducible form of 6-phosphofructose-2-kinase # EP-2702043-A1
CHEMBL3731989 # 6-(1,3-benzothiazol-6-yl)-N-[(1S)-1-{[1,1′-biphenyl]-3-yl}ethyl]-2-methylpyrimidin-4-amine # PATENT
# Inhibitors of inducible form of 6-phosphofructose-2-kinase # EP-2702043-A1
CHEMBL3732016 # 6-(3-methoxy-4-methylphenyl)-2-methyl-N-(1-{3-[(1-methyl-1H-pyrazol-3-
yl)methoxy]phenyl}ethyl)pyrimidin-4-amine # PATENT # Inhibitors of inducible form of 6-phosphofructose-2-kinase # EP-
2702043-A1
CHEMBL3732035 # 6-(1,3-benzothiazol-6-yl)-N-(2,2-dimethyl-3,4-dihydro-2H-1-benzopyran-4-yl)-2-methylpyrimidin-4-
amine # PATENT # Inhibitors of inducible form of 6-phosphofructose-2-kinase # EP-2702043-A1
CHEMBL3732067 # 6-(1,3-benzothiazol-6-yl)-N-(1-{5-[(1,3-dimethyl-1H-pyrazol-5-yl)methoxy]pyridin-3-yl}ethyl)-2-
methylpyrimidin-4-amine # PATENT # Inhibitors of inducible form of 6-phosphofructose-2-kinase # EP-2702043-A1
CHEMBL3732068 # 6-(1,3-benzothiazol-6-yl)-N-[(1S)-1-(3-bromophenyl)ethyl]-2-methylpyrimidin-4-amine # PATENT #
Inhibitors of inducible form of 6-phosphofructose-2-kinase # EP-2702043-A1
CHEMBL3732121 # 2-methyl-6-(3-methyl-1-benzofuran-5-yl)-N-[(1S)-1-(3-{4-methyl-2H,3H,4H-pyrido[3,2-b][1,4]oxazin-
7-yl}phenyl)ethyl]pyrimidin-4-amine # PATENT # Inhibitors of inducible form of 6-phosphofructose-2-kinase # EP-
2702043-A1
CHEMBL3732122 # 6-(3,4-difluorophenyl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3732145 # 4-[3-(1-{[6-(1,3-benzothiazol-6-yl)-2-methylpyrimidin-4-yl]amino}ethyl)phenoxy]butan-1-ol # PATENT
# Inhibitors of inducible form of 6-phosphofructose-2-kinase # EP-2702043-A1
CHEMBL3732162 # 6-(1,3-benzothiazol-6-yl)-N-[(1S)-1-{3-[2-(diethylamino)pyrimidin-5-yl]phenyl}ethyl]-2-
methylpyrimidin-4-amine # PATENT # Inhibitors of inducible form of 6-phosphofructose-2-kinase # EP-2702043-A1
CHEMBL3732165 # 6-(1,3-benzothiazol-6-yl)-2-methyl-N-[(1S)-1-[3-(6-methylpyridin-3-yl)phenyl]ethyl]pyrimidin-4-amine
# PATENT # Inhibitors of inducible form of 6-phosphofructose-2-kinase # EP-2702043-A1
CHEMBL3732184 # 6-(1,3-benzothiazol-6-yl)-N-[1-(3-bromophenyl)ethyl]-2-methylpyrimidin-4-amine # PATENT #
Inhibitors of inducible form of 6-phosphofructose-2-kinase # EP-2702043-A1
CHEMBL3732203 # 6-(1,3-benzothiazol-6-yl)-N-[(1S)-1-[3-(6-fluoro-5-methylpyridin-3-yl)phenyl]ethyl]-2-methylpyrimidin-
4-amine # PATENT # Inhibitors of inducible form of 6-phosphofructose-2-kinase # EP-2702043-A1
CHEMBL3732221 # 2-[3-(1-{[2-methyl-6-(3-methyl-1-benzofuran-5-yl)pyrimidin-4-yl]amino}ethyl)phenoxy]acetamide #
PATENT # Inhibitors of inducible form of 6-phosphofructose-2-kinase # EP-2702043-A1
CHEMBL3732230 # 6-(2-chloro-3-methoxyphenyl)-2-methyl-N-[(1R)-1,2,3,4-tetrahydronaphthalen-1-yl]pyrimidin-4-amine
# PATENT # Inhibitors of inducible form of 6-phosphofructose-2-kinase # EP-2702043-A1
CHEMBL3732242 # N,N-dimethyl-2-{3-[(1R)-1-{[2-methyl-6-(3-methyl-1-benzofuran-5-yl)pyrimidin-4-
yl]amino}ethyl]phenoxy}acetamide # PATENT # Inhibitors of inducible form of 6-phosphofructose-2-kinase # EP-
2702043-A1
CHEMBL3732249 # N-[3-(1-{[6-(1,3-benzothiazol-6-yl)-2-methylpyrimidin-4-yl]amino}ethyl)phenyl]-1-methyl-1H-pyrazole-
3-carboxamide # PATENT # Inhibitors of inducible form of 6-phosphofructose-2-kinase # EP-2702043-A1
CHEMBL3732257 # 6-(1,3-benzothiazol-6-yl)-2-methyl-N-(1-{3-[(1H-pyrazol-5-yl)methoxy]phenyl}ethyl)pyrimidin-4-amine
# PATENT # Inhibitors of inducible form of 6-phosphofructose-2-kinase # EP-2702043-A1
CHEMBL3732258 # N-[(1S)-1-[3-(2-aminopyrimidin-5-yl)phenyl]ethyl]-6-(3-ethyl-1-benzofuran-5-yl)-2-methylpyrimidin-4-
amine # PATENT # Inhibitors of inducible form of 6-phosphofructose-2-kinase # EP-2702043-A1
CHEMBL3732261 # 2-[3-(1-{[6-(1,3-benzothiazol-6-yl)-2-methylpyrimidin-4-yl]amino}ethyl)phenoxy]-N-
cyclopropylacetamide # PATENT # Inhibitors of inducible form of 6-phosphofructose-2-kinase # EP-2702043-A1
CHEMBL3732274 # 2-methyl-6-(1-methyl-1H-indol-5-yl)-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3732306 # 1-(5-{3-[(1S)-1-{[6-(4-chloro-3-fluorophenyl)-2-methylpyrimidin-4-yl]amino}ethyl]phenyl}pyrimidin-2-
yl)piperidin-4-ol # PATENT # Inhibitors of inducible form of 6-phosphofructose-2-kinase # EP-2702043-A1
CHEMBL3732333 # 6-(1,3-benzothiazol-6-yl)-2-methyl-N-(1-{3-[6-(piperazin-1-yl)pyridin-3-yl]phenyl}ethyl)pyrimidin-4-
amine # PATENT # Inhibitors of inducible form of 6-phosphofructose-2-kinase # EP-2702043-A1
CHEMBL3732337 # 6-(1,3-benzothiazol-6-yl)-2-methyl-N-(1-{3-[3-(pyrrolidin-1-yl)propoxy]phenyl}ethyl)pyrimidin-4-amine
# PATENT # Inhibitors of inducible form of 6-phosphofructose-2-kinase # EP-2702043-A1
CHEMBL3732363 # 6-(1-benzothiophen-5-yl)-N-[(1S)-1-(3-methoxyphenyl)ethyl]-2-methylpyrimidin-4-amine # PATENT #
Inhibitors of inducible form of 6-phosphofructose-2-kinase # EP-2702043-A1
CHEMBL3732384 # 6-(4-chloro-3-methylphenyl)-2-methyl-N-{1-[3-(1-methyl-1H-pyrazol-4-yl)phenyl]ethyl}pyrimidin-4-
amine # PATENT # Inhibitors of inducible form of 6-phosphofructose-2-kinase # EP-2702043-A1
CHEMBL3732385 # N-[(1S)-1-[3-(2-aminopyrimidin-5-yl)phenyl]ethyl]-2-methyl-6-(3-methyl-1-benzothiophen-5-
yl)pyrimidin-4-amine # PATENT # Inhibitors of inducible form of 6-phosphofructose-2-kinase # EP-2702043-A1
CHEMBL3732390 # 2-methyl-6-(3-methyl-1-benzofuran-5-yl)-N-[(1S)-1-[3-(pyridin-3-yl)phenyl]ethyl]pyrimidin-4-amine #
PATENT # Inhibitors of inducible form of 6-phosphofructose-2-kinase # EP-2702043-A1
CHEMBL3732406 # 6-(1,3-benzothiazol-6-yl)-2-methyl-N-{[1-(1-methylcyclopropanecarbonyl)piperidin-3-
yl]methyl}pyrimidin-4-amine # PATENT # Inhibitors of inducible form of 6-phosphofructose-2-kinase # EP-2702043-A1
CHEMBL3732427 # 6-(1,3-benzothiazol-6-yl)-2-methyl-N-(2-methyl-1,2,3,4-tetrahydronaphthalen-1-yl)pyrimidin-4-amine
# PATENT # Inhibitors of inducible form of 6-phosphofructose-2-kinase # EP-2702043-A1
CHEMBL3732442 # N-[(1S)-1-(3-methoxyphenyl)ethyl]-2-methyl-6-(4-methylphenyl)pyrimidin-4-amine # PATENT #
Inhibitors of inducible form of 6-phosphofructose-2-kinase # EP-2702043-A1
CHEMBL3732462 # 5′-(1-{[2-methyl-6-(3-methyl-1-benzofuran-5-yl)pyrimidin-4-yl]amino}ethyl)-[3,3′-bipyridin]-5-amine #
PATENT # Inhibitors of inducible form of 6-phosphofructose-2-kinase # EP-2702043-A1
CHEMBL3732478 # 6-(1,3-benzothiazol-6-yl)-N-[(1S)-1-[3-(6-chloropyridin-3-yl)phenyl]ethyl]-2-methylpyrimidin-4-amine #
PATENT # Inhibitors of inducible form of 6-phosphofructose-2-kinase # EP-2702043-A1
CHEMBL3732481 # 2-[3-(1-{[2-methyl-6-(1-methyl-1H-indol-6-yl)pyrimidin-4-yl]amino}ethyl)phenoxy]-1-(morpholin-4-
yl)ethan-1-one # PATENT # Inhibitors of inducible form of 6-phosphofructose-2-kinase # EP-2702043-A1
CHEMBL3732484 # 2-methyl-N-{1-[3-(1-methyl-1H-pyrazol-4-yl)phenyl]ethyl}-6-[4-(propan-2-yl)phenyl]pyrimidin-4-amine
# PATENT # Inhibitors of inducible form of 6-phosphofructose-2-kinase # EP-2702043-A1
CHEMBL3732494 # 1-[3-(1-{[2-methyl-6-(3-methyl-1-benzofuran-5-yl)pyrimidin-4-yl]amino}ethyl)phenoxy]propan-2-one #
PATENT # Inhibitors of inducible form of 6-phosphofructose-2-kinase # EP-2702043-A1
CHEMBL3732506 # 6-(3,4-dihydro-2H-1,4-benzoxazin-6-yl)-2-methyl-N-[(1R)-1,2,3,4-tetrahydronaphthalen-1-
yl]pyrimidin-4-amine # PATENT # Inhibitors of inducible form of 6-phosphofructose-2-kinase # EP-2702043-A1
CHEMBL3732515 # N-{1-[2-(2-aminopyrimidin-5-yl)pyridin-4-yl]ethyl}-2-methyl-6-(3-methyl-1-benzofuran-5-yl)pyrimidin-
4-amine # PATENT # Inhibitors of inducible form of 6-phosphofructose-2-kinase # EP-2702043-A1
CHEMBL3732530 # N-[(1S)-1-[3-(2-aminopyrimidin-5-yl)phenyl]ethyl]-6-(7-fluoro-1-benzofuran-5-yl)-2-methylpyrimidin-4-
amine # PATENT # Inhibitors of inducible form of 6-phosphofructose-2-kinase # EP-2702043-A1
CHEMBL3732537 # N-[(1S)-1-[3-(2-{[(furan-3-yl)methyl]amino}pyrimidin-5-yl)phenyl]ethyl]-2-methyl-6-(3-methyl-1-
benzofuran-5-yl)pyrimidin-4-amine # PATENT # Inhibitors of inducible form of 6-phosphofructose-2-kinase # EP-
2702043-A1
CHEMBL3732540 # N-[1-(3-bromo-5-fluorophenyl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3732548 # 1-(5-{3-[(1S)-1-{[6-(7-fluoro-3-methyl-1-benzofuran-5-yl)-2-methylpyrimidin-4-
yl]amino}ethyl]phenyl}pyrimidin-2-yl)piperidin-4-ol # PATENT # Inhibitors of inducible form of 6-phosphofructose-2-kinase
# EP-2702043-A1
CHEMBL3732555 # 5-{3-[(1S)-1-{[2-methyl-6-(3-methyl-1-benzofuran-5-yl)pyrimidin-4-yl]amino}ethyl]phenyl}-1,2-
dihydropyrimidin-2-one # PATENT # Inhibitors of inducible form of 6-phosphofructose-2-kinase # EP-2702043-A1
CHEMBL3732562 # 4-[3-(1-{[2-methyl-6-(1-methyl-1H-indol-6-yl)pyrimidin-4-yl]amino}ethyl)phenoxy]butanoic acid #
PATENT # Inhibitors of inducible form of 6-phosphofructose-2-kinase # EP-2702043-A1
CHEMBL3732567 # 6-(1,3-benzothiazol-6-yl)-2-methyl-N-(1-{3-[3-(morpholin-4-yl)propoxy]phenyl}ethyl)pyrimidin-4-amine
# PATENT # Inhibitors of inducible form of 6-phosphofructose-2-kinase # EP-2702043-A1
CHEMBL3732570 # N,N-diethyl-2-[3-(1-{[2-methyl-6-(naphthalen-2-yl)pyrimidin-4-yl]amino}ethyl)phenoxy]acetamide #
PATENT # Inhibitors of inducible form of 6-phosphofructose-2-kinase # EP-2702043-A1
CHEMBL3732580 # N-[1-(3-bromo-4-methylphenyl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3732581 # 6-(1,3-benzothiazol-6-yl)-N-(1-{3-[3-(dimethylamino)propoxy]phenyl}ethyl)-2-methylpyrimidin-4-
amine # PATENT # Inhibitors of inducible form of 6-phosphofructose-2-kinase # EP-2702043-A1
CHEMBL3732595 # 6-(4-chlorophenyl)-2-methyl-N-[(1R)-1,2,3,4-tetrahydronaphthalen-1-yl]pyrimidin-4-amine # PATENT
# Inhibitors of inducible form of 6-phosphofructose-2-kinase # EP-2702043-A1
CHEMBL3732607 # 6-(1,3-benzothiazol-6-yl)-N-{1-[3-(2,6-dimethylpyridin-3-yl)phenyl]ethyl}-2-methylpyrimidin-4-amine #
PATENT # Inhibitors of inducible form of 6-phosphofructose-2-kinase # EP-2702043-A1
CHEMBL3732610 # 2-[3-(1-{[6-(1,3-benzothiazol-6-yl)-2-methylpyrimidin-4-yl]amino}ethyl)phenoxy]-2-
methylpropanamide # PATENT # Inhibitors of inducible form of 6-phosphofructose-2-kinase # EP-2702043-A1
CHEMBL3732635 # 2-[3-(1-{[6-(1,3-benzothiazol-6-yl)-2-methylpyrimidin-4-yl]amino}ethyl)phenoxy]-N-(propan-2-
yl)acetamide # PATENT # Inhibitors of inducible form of 6-phosphofructose-2-kinase # EP-2702043-A1
CHEMBL3732641 # 2-methyl-6-(3-methyl-1-benzofuran-5-yl)-N-[(1S)-1-[3-(1H-pyrazol-1-yl)phenyl]ethyl]pyrimidin-4-
amine # PATENT # Inhibitors of inducible form of 6-phosphofructose-2-kinase # EP-2702043-A1
CHEMBL3732679 # 6-(1,3-benzothiazol-6-yl)-2-methyl-N-[(1S)-1-[3-(pyrimidin-5-yl)phenyl]ethyl]pyrimidin-4-amine #
PATENT # Inhibitors of inducible form of 6-phosphofructose-2-kinase # EP-2702043-A1
CHEMBL3732692 # 2-methyl-6-(3-methyl-1-benzofuran-5-yl)-N-{1-[5-(1-methyl-1H-pyrazol-4-yl)pyridin-3-
yl]ethyl}pyrimidin-4-amine # PATENT # Inhibitors of inducible form of 6-phosphofructose-2-kinase # EP-2702043-A1
CHEMBL3732701 # N-[(1S)-1-[4-fluoro-3-(1-methyl-1H-pyrazol-4-yl)phenyl]ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3732712 # 1-[3-(1-{[6-(1,3-benzothiazol-6-yl)-2-methylpyrimidin-4-yl]amino}ethyl)phenoxy]propan-2-one #
PATENT # Inhibitors of inducible form of 6-phosphofructose-2-kinase # EP-2702043-A1
CHEMBL3732714 # 6-(1,3-benzothiazol-6-yl)-2-methyl-N-(1-{3-[2-(2-methyl-1H-imidazol-1-
yl)ethoxy]phenyl}ethyl)pyrimidin-4-amine # PATENT # Inhibitors of inducible form of 6-phosphofructose-2-kinase # EP-
2702043-A1
CHEMBL3732715 # N-[(1S)-1-[3-(2-aminopyrimidin-5-yl)phenyl]ethyl]-2-methyl-6-(3-methyl-1-benzofuran-5-yl)pyrimidin-
4-amine # PATENT # Inhibitors of inducible form of 6-phosphofructose-2-kinase # EP-2702043-A1
CHEMBL3732722 # N-[(1S)-1-[3-(2-aminopyrimidin-5-yl)phenyl]ethyl]-6-(4-chloro-3-fluorophenyl)-2-methylpyrimidin-4-
amine # PATENT # Inhibitors of inducible form of 6-phosphofructose-2-kinase # EP-2702043-A1
CHEMBL3732742 # 2,2-dimethyl-3-[(5-{3-[(1S)-1-{[2-methyl-6-(3-methyl-1-benzofuran-5-yl)pyrimidin-4-
yl]amino}ethyl]phenyl}pyrimidin-2-yl)amino]propan-1-ol # PATENT # Inhibitors of inducible form of 6-phosphofructose-2-
kinase # EP-2702043-A1
CHEMBL3732745 # 6-(1,3-benzothiazol-6-yl)-2-methyl-N-(1-{3-[2-(1H-pyrazol-1-yl)ethoxy]phenyl}ethyl)pyrimidin-4-amine
# PATENT # Inhibitors of inducible form of 6-phosphofructose-2-kinase # EP-2702043-A1
CHEMBL3732772 # N-(1-{3-[(1,3-dimethyl-1H-pyrazol-5-yl)methoxy]phenyl}ethyl)-6-(3-ethyl-1-benzofuran-5-yl)-2-
methylpyrimidin-4-amine # PATENT # Inhibitors of inducible form of 6-phosphofructose-2-kinase # EP-2702043-A1
CHEMBL3732782 # N-[(1S)-1-[3-(2-aminopyrimidin-5-yl)phenyl]ethyl]-6-(4-chloro-3,5-difluorophenyl)-2-methylpyrimidin-
4-amine # PATENT # Inhibitors of inducible form of 6-phosphofructose-2-kinase # EP-2702043-A1
CHEMBL3732789 # N-[(1S)-1-{3-[2-(4-ethylpiperazin-1-yl)pyrimidin-5-yl]phenyl]ethyl]-2-methyl-6-(3-methyl-1-benzofuran-
5-yl)pyrimidin-4-amine # PATENT # Inhibitors of inducible form of 6-phosphofructose-2-kinase # EP-2702043-A1
CHEMBL3732820 # N,N-diethyl-2-[3-(1-{[2-methyl-6-(3-methyl-1-benzofuran-5-yl)pyrimidin-4-
yl]amino}ethyl)phenoxy]acetamide # PATENT # Inhibitors of inducible form of 6-phosphofructose-2-kinase # EP-
2702043-A1
CHEMBL3732822 # N-[(1R)-1-[4-fluoro-3-(1-methyl-1H-pyrazol-4-yl)phenyl]ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3732828 # 6-(7-fluoro-3-methyl-1-benzofuran-5-yl)-N-[(1S)-1-[3-(6-fluoropyridin-3-yl]phenyl]ethyl]-2-
methylpyrimidin-4-amine # PATENT # Inhibitors of inducible form of 6-phosphofructose-2-kinase # EP-2702043-A1
CHEMBL3732835 # 2-[3-(1-{[6-(1,3-benzothiazol-6-yl)-2-methylpyrimidin-4-yl]amino}ethyl)phenoxy]-1-(piperidin-1-
yl)ethan-1-one # PATENT # Inhibitors of inducible form of 6-phosphofructose-2-kinase # EP-2702043-A1
CHEMBL3732847 # 6-(1-benzofuran-5-yl)-2-methyl-N-(1-{3-[(1-methyl-1H-pyrazol-3-yl)methoxy]phenyl}ethyl)pyrimidin-4-
amine # PATENT # Inhibitors of inducible form of 6-phosphofructose-2-kinase # EP-2702043-A1
CHEMBL3732850 # 6-(2-chloro-3-methoxyphenyl)-N-(2,3-dihydro-1H-inden-1-yl)-2-methylpyrimidin-4-amine # PATENT #
Inhibitors of inducible form of 6-phosphofructose-2-kinase # EP-2702043-A1
CHEMBL3732874 # 3-(1-{[6-(3-methoxy-4-methylphenyl)-2-methylpyrimidin-4-yl]amino}ethyl)phenol # PATENT #
Inhibitors of inducible form of 6-phosphofructose-2-kinase # EP-2702043-A1
CHEMBL3732896 # N-[(1S)-1-{[1,1′-biphenyl]-3-yl]ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3732906 # 6-(2-chloro-3-methoxyphenyl)-N-[(1S)-1-(3-methoxyphenyl)ethyl]-2-methylpyrimidin-4-amine #
PATENT # Inhibitors of inducible form of 6-phosphofructose-2-kinase # EP-2702043-A1
CHEMBL3732914 # 2-[3-(1-{[6-(1,3-benzothiazol-6-yl)-2-methylpyrimidin-4-yl]amino}ethyl)phenoxy]-N-(2-
hydroxypropyl)acetamide # PATENT # Inhibitors of inducible form of 6-phosphofructose-2-kinase # EP-2702043-A1
CHEMBL3732915 # 6-(1,3-benzothiazol-6-yl)-N-[(1S)-1-[3-(5-fluoropyridin-3-yl)phenyl]ethyl]-2-methylpyrimidin-4-amine #
PATENT # Inhibitors of inducible form of 6-phosphofructose-2-kinase # EP-2702043-A1
CHEMBL3732922 # 3-[(1S)-1-{[6-(1,3-benzothiazol-6-yl)-2-methylpyrimidin-4-yl]amino}ethyl]phenol # PATENT #
Inhibitors of inducible form of 6-phosphofructose-2-kinase # EP-2702043-A1
CHEMBL3732926 # 3-(1-{[6-(1,3-benzothiazol-6-yl)-2-methylpyrimidin-4-yl]amino}ethyl)benzene-1-carboximidamide #
PATENT # Inhibitors of inducible form of 6-phosphofructose-2-kinase # EP-2702043-A1
CHEMBL3732935 # N-[(1S)-1-[3-(2-{4-[2-(dimethylamino)ethyl]piperazin-1-yl}pyrimidin-5-yl)phenyl]ethyl]-2-methyl-6-(3-
methyl-1-benzofuran-5-yl)pyrimidin-4-amine # PATENT # Inhibitors of inducible form of 6-phosphofructose-2-kinase #
EP-2702043-A1
CHEMBL3732936 # 2-methyl-6-(3-methyl-1-benzofuran-5-yl)-N-[(1S)-1-[3-(pyrimidin-5-yl)phenyl]ethyl]pyrimidin-4-amine
# PATENT # Inhibitors of inducible form of 6-phosphofructose-2-kinase # EP-2702043-A1
CHEMBL3732942 # N-[(1S)-1-[5-(2-aminopyrimidin-5-yl)pyridin-3-yl]ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3732946 # 6-(4-chloro-3-methylphenyl)-2-methyl-N-[(1R)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3732968 # 2-methyl-6-[4-methyl-3-(prop-2-yn-1-yloxy)phenyl]-N-[(1R)-1,2,3,4-tetrahydronaphthalen-1-
yl]pyrimidin-4-amine # PATENT # Inhibitors of inducible form of 6-phosphofructose-2-kinase # EP-2702043-A1
CHEMBL3732994 # 6-(1,3-benzothiazol-6-yl)-2-methyl-N-{1-[3-(4H-1,2,4-triazol-4-yl)phenyl]ethyl}pyrimidin-4-amine #
PATENT # Inhibitors of inducible form of 6-phosphofructose-2-kinase # EP-2702043-A1
CHEMBL3733001 # 6-(1,3-benzothiazol-6-yl)-N-[1-(2-methoxypyridin-4-yl)ethyl]-2-methylpyrimidin-4-amine # PATENT #
Inhibitors of inducible form of 6-phosphofructose-2-kinase # EP-2702043-A1
CHEMBL3733003 # N-[(1S)-1-(4-fluorophenyl)ethyl]-2-methyl-6-(1-methyl-1H-indol-6-yl)pyrimidin-4-amine # PATENT #
Inhibitors of inducible form of 6-phosphofructose-2-kinase # EP-2702043-A1
CHEMBL3733010 # N-[(1S)-1-[3-(5-fluoropyridin-3-yl)phenyl]ethyl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3733018 # N-[(1S)-1-{3-[6-(dimethylamino)pyridin-3-yl]phenyl]ethyl]-2-methyl-6-(3-methyl-1-benzofuran-5-
yl)pyrimidin-4-amine # PATENT # Inhibitors of inducible form of 6-phosphofructose-2-kinase # EP-2702043-A1
CHEMBL3733029 # 6-(1,3-benzothiazol-6-yl)-N-[2-(cyclohex-1-en-1-yl)ethyl]-2-methylpyrimidin-4-amine # PATENT #
Inhibitors of inducible form of 6-phosphofructose-2-kinase # EP-2702043-A1
CHEMBL3733031 # 1-[3-(1-{[6-(1,3-benzothiazol-6-yl)-2-methylpyrimidin-4-yl]amino}ethyl)phenoxy]-3-fluoropropan-2-ol #
PATENT # Inhibitors of inducible form of 6-phosphofructose-2-kinase # EP-2702043-A1
CHEMBL3733041 # N-[(1S)-1-[3-(2-aminopyrimidin-5-yl)phenyl]ethyl]-6-(3-chloro-1-benzofuran-5-yl)-2-methylpyrimidin-4-
amine # PATENT # Inhibitors of inducible form of 6-phosphofructose-2-kinase # EP-2702043-A1
CHEMBL3733056 # N-[(4R)-3,4-dihydro-2H-1-benzopyran-4-yl]-2-methyl-6-(3-methyl-1-benzofuran-5-yl)pyrimidin-4-
amine # PATENT # Inhibitors of inducible form of 6-phosphofructose-2-kinase # EP-2702043-A1
CHEMBL3733060 # 6-(3-fluorophenyl)-N-[(1S)-1-(3-methoxyphenyl)ethyl]-2-methylpyrimidin-4-amine # PATENT #
Inhibitors of inducible form of 6-phosphofructose-2-kinase # EP-2702043-A1
CHEMBL3733066 # 6-(1,3-benzothiazol-6-yl)-2-methyl-N-{1-[3-(1-methyl-1H-pyrazol-4-yl)phenyl]ethyl}pyrimidin-4-amine
# PATENT # Inhibitors of inducible form of 6-phosphofructose-2-kinase # EP-2702043-A1
CHEMBL3733075 # 2-{[2-fluoro-5-(2-methyl-6-{[(1R)-1,2,3,4-tetrahydronaphthalen-1-yl]amino}pyrimidin-4-
yl)phenyl]amino}acetonitrile # PATENT # Inhibitors of inducible form of 6-phosphofructose-2-kinase # EP-2702043-A1
CHEMBL3733107 # 6-(2,5-dimethylphenyl)-2-methyl-N-{1-[3-(1-methyl-1H-pyrazol-4-yl)phenyl]ethyl}pyrimidin-4-amine #
PATENT # Inhibitors of inducible form of 6-phosphofructose-2-kinase # EP-2702043-A1
CHEMBL3733124 # 6-(1,3-benzothiazol-6-yl)-N-(1-{4-fluoro-3-[(1-methyl-1H-pyrazol-5-yl)methoxy]phenyl}ethyl)-2-
methylpyrimidin-4-amine # PATENT # Inhibitors of inducible form of 6-phosphofructose-2-kinase # EP-2702043-A1
CHEMBL3733132 # 6-(1,3-benzothiazol-6-yl)-N-(1-{3-[3-(diethylamino)propoxy]phenyl}ethyl)-2-methylpyrimidin-4-amine
# PATENT # Inhibitors of inducible form of 6-phosphofructose-2-kinase # EP-2702043-A1
CHEMBL3733133 # 6-(1,3-benzothiazol-6-yl)-2-methyl-N-[(1S)-1,2,3,4-tetrahydronaphthalen-1-yl]pyrimidin-4-amine #
PATENT # Inhibitors of inducible form of 6-phosphofructose-2-kinase # EP-2702043-A1
CHEMBL3733143 # 6-(1,3-benzothiazol-6-yl)-2-methyl-N-[1-(3-propoxyphenyl)ethyl]pyrimidin-4-amine # PATENT #
Inhibitors of inducible form of 6-phosphofructose-2-kinase # EP-2702043-A1
CHEMBL3733158 # 6-(4-chloro-3-fluorophenyl)-N-[(1S)-1-[3-(1-ethyl-1H-pyrazol-4-yl)phenyl]ethyl]-2-methylpyrimidin-4-
amine # PATENT # Inhibitors of inducible form of 6-phosphofructose-2-kinase # EP-2702043-A1
CHEMBL3733179 # 2-fluoro-4-[2-methyl-6-({1-[3-(1-methyl-1H-pyrazol-4-yl)phenyl]ethyl}amino)pyrimidin-4-yl]benzamide
# PATENT # Inhibitors of inducible form of 6-phosphofructose-2-kinase # EP-2702043-A1
CHEMBL3733180 # 2-[3-(1-{[6-(1,3-benzothiazol-6-yl)-2-methylpyrimidin-4-yl]amino}ethyl)phenoxy]-N,N-
diethylacetamide # PATENT # Inhibitors of inducible form of 6-phosphofructose-2-kinase # EP-2702043-A1
CHEMBL3733196 # 2-methyl-6-(3-methyl-1-benzofuran-5-yl)-N-[(1S)-1-[5-(1H-pyrazol-4-yl)pyridin-3-yl]ethyl]pyrimidin-4-
amine # PATENT # Inhibitors of inducible form of 6-phosphofructose-2-kinase # EP-2702043-A1
CHEMBL3733199 # N-[(1S)-1-(4-fluorophenyl)ethyl]-2-methyl-6-(3-methyl-1-benzofuran-5-yl)pyrimidin-4-amine #
PATENT # Inhibitors of inducible form of 6-phosphofructose-2-kinase # EP-2702043-A1
CHEMBL3733202 # 6-(furan-3-yl)-2-methyl-N-[(1R)-1,2,3,4-tetrahydronaphthalen-1-yl]pyrimidin-4-amine # PATENT #
Inhibitors of inducible form of 6-phosphofructose-2-kinase # EP-2702043-A1
CHEMBL3733211 # 1-(azepan-1-yl)-2-[3-(1-{[6-(1,3-benzothiazol-6-yl)-2-methylpyrimidin-4-
yl]amino}ethyl)phenoxy]ethan-1-one # PATENT # Inhibitors of inducible form of 6-phosphofructose-2-kinase # EP-
2702043-A1
CHEMBL3733257 # 6-(1,3-benzothiazol-6-yl)-N-{1-[3-(2-fluoropyridin-3-yl)phenyl]ethyl}-2-methylpyrimidin-4-amine #
PATENT # Inhibitors of inducible form of 6-phosphofructose-2-kinase # EP-2702043-A1
CHEMBL3733262 # N-[(4R)-3,4-dihydro-2H-1-benzopyran-4-yl]-2-methyl-6-(1-methyl-1H-indol-6-yl)pyrimidin-4-amine #
PATENT # Inhibitors of inducible form of 6-phosphofructose-2-kinase # EP-2702043-A1
CHEMBL3733265 # N-[1-(3-bromophenyl)ethyl]-6-(2-chloro-3-methoxyphenyl)-2-methylpyrimidin-4-amine # PATENT #
Inhibitors of inducible form of 6-phosphofructose-2-kinase # EP-2702043-A1
CHEMBL3733267 # 6-(1,3-benzothiazol-6-yl)-N-{1-[3-(5-methoxypyridin-3-yl)phenyl]ethyl}-2-methylpyrimidin-4-amine #
PATENT # Inhibitors of inducible form of 6-phosphofructose-2-kinase # EP-2702043-A1
CHEMBL3733282 # 2-methyl-6-(3-methyl-1-benzofuran-5-yl)-N-[(1S)-1-[3-(2-{4-[2-(morpholin-4-yl)ethyl]piperazin-1-
yl}pyrimidin-5-yl)phenyl]ethyl]pyrimidin-4-amine # PATENT # Inhibitors of inducible form of 6-phosphofructose-2-kinase #
EP-2702043-A1
CHEMBL3733284 # 6-(2,3-dihydro-1,4-benzodioxin-6-yl)-2-methyl-N-[(1R)-1,2,3,4-tetrahydronaphthalen-1-yl]pyrimidin-4-
amine # PATENT # Inhibitors of inducible form of 6-phosphofructose-2-kinase # EP-2702043-A1
CHEMBL3733301 # 6-(3-ethylphenyl)-N-[(1S)-1-(3-methoxyphenyl)ethyl]-2-methylpyrimidin-4-amine # PATENT #
Inhibitors of inducible form of 6-phosphofructose-2-kinase # EP-2702043-A1
CHEMBL3733312 # 6-(1,3-benzothiazol-6-yl)-N-[(1S)-1-{3-[(1,3-dimethyl-1H-pyrazol-5-yl)methoxy]phenyl}ethyl]-2-
methylpyrimidin-4-amine # PATENT # Inhibitors of inducible form of 6-phosphofructose-2-kinase # EP-2702043-A1
CHEMBL3733331 # 6-(4-chlorophenyl)-N-[(1S)-1-(3-methoxyphenyl)ethyl]-2-methylpyrimidin-4-amine # PATENT #
Inhibitors of inducible form of 6-phosphofructose-2-kinase # EP-2702043-A1
CHEMBL3733357 # 2-methyl-6-(1-methyl-1H-indol-6-yl)-N-{1-[3-(1-methyl-1H-pyrazol-4-yl)phenyl]ethyl}pyrimidin-4-
amine # PATENT # Inhibitors of inducible form of 6-phosphofructose-2-kinase # EP-2702043-A1
CHEMBL376280 # 2-iodoacetic acid # PUBLICATION # Targeting the Warburg Effect in cancer; relationships for 2-
arylpyridazinones as inhibitors of the key glycolytic enzyme 6-phosphofructo-2-kinase/2,6-bisphosphatase 3 (PFKFB3). #
10.1016/j.bmc.2013.12.041
Kancera_Example-001 # 2,3,4-trichloro-N-[3-(2H-1,2,3,4-tetrazol-5-yl)phenyl]benzene-1-sulfonamide # PATENT # New
compounds # WO-2012035171-A2
Kancera_Example-002 # 5-(1,3-oxazol-5-yl)-N-[3-(2H-1,2,3,4-tetrazol-5-yl)phenyl]thiophene-2-sulfonamide # PATENT #
New compounds # WO-2012035171-A2
Kancera_Example-003 # 5-chloro-3-methyl-N-[3-(1,3-oxazol-5-yl)phenyl]-1-benzothiophene-2-sulfonamide # PATENT #
New compounds # WO-2012035171-A2
Kancera_Example-004 # 3-methyl-5-(propan-2-yl)-N-[3-(2H-1,2,3,4-tetrazol-5-yl)phenyl]-1-benzothiophene-2-sulfonamide
# PATENT # New compounds # WO-2012035171-A2
Kancera_Example-005 # 3-[3-methyl-5-(propan-2-yl)-1-benzothiophene-2-sulfonamido]benzoic acid # PATENT # New
compounds # WO-2012035171-A2
Kancera_Example-006 # 2-[3-methyl-5-(propan-2-yl)-1-benzothiophene-2-sulfonamido]pyridine-4-carboxylic acid #
PATENT # New compounds # WO-2012035171-A2
Kancera_Example-007 # 2-[3-methyl-5-(propan-2-yl)-1-benzothiophene-2-sulfonamido]-1,3-thiazole-5-carboxylic acid #
PATENT # New compounds # WO-2012035171-A2
Kancera_Example-008 # 4-methyl-2-[3-methyl-5-(propan-2-yl)-1-benzothiophene-2-sulfonamido]-1,3-thiazole-5-
carboxylic acid # PATENT # New compounds # WO-2012035171-A2
Kancera_Example-009 # 5-[3-methyl-5-(propan-2-yl)-1-benzothiophene-2-sulfonamido]pyridine-3-carboxylic acid #
PATENT # New compounds # WO-2012035171-A2
Kancera_Example-010 # 6-[3-methyl-5-(propan-2-yl)-1-benzothiophene-2-sulfonamido]pyridine-2-carboxylic acid #
PATENT # New compounds # WO-2012035171-A2
Kancera_Example-011 # 3-[3-methyl-5-(propan-2-yl)-1-benzothiophene-2-sulfonamido]-5-nitrobenzoic acid # PATENT #
New compounds # WO-2012035171-A2
Kancera_Example-012 # 2-(5-{3-[3-methyl-5-(propan-2-yl)-1-benzothiophene-2-sulfonamido]phenyl}-2H-1,2,3,4-tetrazol-
2-yl)acetic acid # PATENT # New compounds # WO-2012035171-A2
Kancera_Example-013 # 3-methyl-5-(propan-2-yl)-N-[3-(2H-1,2,3,4-tetrazol-5-yl)phenyl]-1-benzofuran-2-sulfonamide #
PATENT # New compounds # WO-2012035171-A2
Kancera_Example-014 # 3-[3-methyl-5-(propan-2-yl)-1-benzofuran-2-sulfonamido]benzoic acid # PATENT # New
compounds # WO-2012035171-A2
Kancera_Example-015 # 2-{3-[3-methyl-5-(propan-2-yl)-1-benzothiophene-2-sulfonamido]phenyl}acetic acid # PATENT #
New compounds # WO-2012035171-A2
Kancera_Example-016 # N-[3-(2H-1,2,3,4-tetrazol-5-yl)phenyl]-[1,1′-biphenyl]-3-sulfonamide # PATENT # New
compounds # WO-2012035171-A2
Kancera_Example-017 # 4′-fluoro-N-[3-(2H-1,2,3,4-tetrazol-5-yl)phenyl]-[1,1′-biphenyl]-3-sulfonamide # PATENT # New
compounds # WO-2012035171-A2
Kancera_Example-018 # 2,6-dichloro-N-[3-(2H-1,2,3,4-tetrazol-5-yl)phenyl]-4-(trifluoromethyl)benzene-1-sulfonamide #
PATENT # New compounds # WO-2012035171-A2
Kancera_Example-019 # 4′-methoxy-N-[3-(2H-1,2,3,4-tetrazol-5-yl)phenyl]-[1,1′-biphenyl]-3-sulfonamide # PATENT #
New compounds # WO-2012035171-A2
Kancera_Example-020 # N-[3-(2H-1,2,3,4-tetrazol-5-yl)phenyl]naphthalene-2-sulfonamide # PATENT # New compounds
# WO-2012035171-A2
Kancera_Example-021 # 5-[2-(methylsulfanyl)pyrimidin-4-yl]-N-[3-(2H-1,2,3,4-tetrazol-5-yl)phenyl]thiophene-2-
sulfonamide # PATENT # New compounds # WO-2012035171-A2
Kancera_Example-022 # 5-(5-fluoro-2-methoxyphenyl)-N-[3-(2H-1,2,3,4-tetrazol-5-yl)phenyl]thiophene-2-sulfonamide #
PATENT # New compounds # WO-2012035171-A2
Kancera_Example-023 # 5-(3,5-difluorophenyl)-N-[3-(2H-1,2,3,4-tetrazol-5-yl)phenyl]thiophene-2-sulfonamide # PATENT
# New compounds # WO-2012035171-A2
Kancera_Example-024 # 5-(2-methoxyphenyl)-N-[3-(2H-1,2,3,4-tetrazol-5-yl)phenyl]thiophene-2-sulfonamide # PATENT
# New compounds # WO-2012035171-A2
Kancera_Example-025 # 5-(2-methyl-1,3-thiazol-4-yl)-N-[3-(2H-1,2,3,4-tetrazol-5-yl)phenyl]thiophene-2-sulfonamide #
PATENT # New compounds # WO-2012035171-A2
Kancera_Example-026 # 5-(2-chlorophenyl)-N-[3-(2H-1,2,3,4-tetrazol-5-yl)phenyl]thiophene-2-sulfonamide # PATENT #
New compounds # WO-2012035171-A2
Kancera_Example-027 # 5-(4-methylphenyl)-N-[3-(2H-1,2,3,4-tetrazol-5-yl)phenyl]thiophene-2-sulfonamide # PATENT #
New compounds # WO-2012035171-A2
Kancera_Example-028 # 5-(2,4-dimethoxyphenyl)-N-[3-(2H-1,2,3,4-tetrazol-5-yl)phenyl]thiophene-2-sulfonamide #
PATENT # New compounds # WO-2012035171-A2
Kancera_Example-029 # 5-(4-chlorophenyl)-N-[3-(2H-1,2,3,4-tetrazol-5-yl)phenyl]thiophene-2-sulfonamide # PATENT #
New compounds # WO-2012035171-A2
Kancera_Example-030 # 5-(4-fluoro-2-methoxyphenyl)-N-[3-(2H-1,2,3,4-tetrazol-5-yl)phenyl]thiophene-2-sulfonamide #
PATENT # New compounds # WO-2012035171-A2
Kancera_Example-031 # N-[3-(2H-1,2,3,4-tetrazol-5-yl)phenyl]-[1,1′-biphenyl]-4-sulfonamide # PATENT # New
compounds # WO-2012035171-A2
Kancera_Example-032 # 5-chloro-3-methyl-N-[3-(2H-1,2,3,4-tetrazol-5-yl)phenyl]-1-benzothiophene-2-sulfonamide #
PATENT # New compounds # WO-2012035171-A2
Kancera_Example-033 # 3,5-dimethyl-N-[3-(2H-1,2,3,4-tetrazol-5-yl)phenyl]-1-benzothiophene-2-sulfonamide # PATENT
# New compounds # WO-2012035171-A2
Kancera_Example-034 # 5-bromo-3-methyl-N-[3-(2H-1,2,3,4-tetrazol-5-yl)phenyl]-1-benzothiophene-2-sulfonamide #
PATENT # New compounds # WO-2012035171-A2
Kancera_Example-035 # 7-methoxy-3-methyl-N-[3-(2H-1,2,3,4-tetrazol-5-yl)phenyl]-1-benzothiophene-2-sulfonamide #
PATENT # New compounds # WO-2012035171-A2
Kancera_Example-036 # 7-chloro-3-methyl-N-[3-(2H-1,2,3,4-tetrazol-5-yl)phenyl]-1-benzothiophene-2-sulfonamide #
PATENT # New compounds # WO-2012035171-A2
Kancera_Example-037 # 5-methoxy-3-methyl-N-[3-(2H-1,2,3,4-tetrazol-5-yl)phenyl]-1-benzothiophene-2-sulfonamide #
PATENT # New compounds # WO-2012035171-A2
Kancera_Example-038 # 3-(5-chloro-3-methyl-1-benzothiophene-2-sulfonamido)benzoic acid # PATENT # New
compounds # WO-2012035171-A2
Kancera_Example-039 # 3-methyl-N-[3-(2H-1,2,3,4-tetrazol-5-yl)phenyl]-1-benzothiophene-2-sulfonamide # PATENT #
New compounds # WO-2012035171-A2
Kancera_Example-040 # 5-methyl-N-[3-(2H-1,2,3,4-tetrazol-5-yl]phenyl]-1-benzothiophene-2-sulfonamide # PATENT #
New compounds # WO-2012035171-A2
Kancera_Example-041 # 3-(5-bromo-3-methyl-1-benzothiophene-2-sulfonamido)benzoic acid # PATENT # New
compounds # WO-2012035171-A2
Kancera_Example-042 # 3-(7-methoxy-3-methyl-1-benzothiophene-2-sulfonamido)benzoic acid # PATENT # New
compounds # WO-2012035171-A2
Kancera_Example-043 # 5-fluoro-3-methyl-N-[3-(2H-1,2,3,4-tetrazol-5-yl)phenyl]-1-benzothiophene-2-sulfonamide #
PATENT # New compounds # WO-2012035171-A2
Kancera_Example-044 # 3-(7-chloro-3-methyl-1-benzothiophene-2-sulfonamido)benzoic acid # PATENT # New
compounds # WO-2012035171-A2
Kancera_Example-045 # 3-methyl-5-(pyrrolidin-1-yl)-N-[3-(2H-1,2,3,4-tetrazol-5-yl)phenyl]-1-benzothiophene-2-
sulfonamide # PATENT # New compounds # WO-2012035171-A2
Kancera_Example-046 # 3-[3-methyl-5-(pyrrolidin-1-yl)-1-benzothiophene-2-sulfonamido]benzoic acid # PATENT # New
compounds # WO-2012035171-A2
Kancera_Example-047 # 3-(5-amino-3-methyl-1-benzothiophene-2-sulfonamido)benzoic acid # PATENT # New
compounds # WO-2012035171-A2
Kancera_Example-048 # 5-amino-3-methyl-N-[3-(2H-1,2,3,4-tetrazol-5-yl]phenyl]-1-benzothiophene-2-sulfonamide #
PATENT # New compounds # WO-2012035171-A2
Kancera_Example-049 # 3-(5-acetamido-3-methyl-1-benzothiophene-2-sulfonamido)benzoic acid # PATENT # New
compounds # WO-2012035171-A2
Kancera_Example-050 # 4′-methyl-N-[3-(2H-1,2,3,4-tetrazol-5-yl)phenyl]-[1,1′-biphenyl]-3-sulfonamide # PATENT # New
compounds # WO-2012035171-A2
Kancera_Example-051 # 2,4-dichloro-5-methyl-N-[3-(2H-1,2,3,4-tetrazol-5-yl)phenyl]benzene-1-sulfonamide # PATENT #
New compounds # WO-2012035171-A2
Kancera_Example-052 # 3′,5′-dichloro-N-[3-(2H-1,2,3,4-tetrazol-5-yl]phenyl]-[1,1′-biphenyl]-3-sulfonamide # PATENT #
New compounds # WO-2012035171-A2
Kancera_Example-053 # 2,4-dichloro-6-methyl-N-[3-(2H-1,2,3,4-tetrazol-5-yl)phenyl]benzene-1-sulfonamide # PATENT #
New compounds # WO-2012035171-A2
Kancera_Example-054 # 3-{3′,5′-dichloro-[1,1′-biphenyl]-3-sulfonamido}benzoic acid # PATENT # New compounds #
WO-2012035171-A2
Kancera_Example-055 # N-[3-(2H-1,2,3,4-tetrazol-5-yl)phenyl]-4′-(trifluoromethyl)-[1,1′-biphenyl]-3-sulfonamide #
PATENT # New compounds # WO-2012035171-A2
Kancera_Example-056 # 4-bromo-N-[3-(2H-1,2,3,4-tetrazol-5-yl)phenyl]-2-(trifluoromethyl)benzene-1-sulfonamide #
PATENT # New compounds # WO-2012035171-A2
Kancera_Example-057 # 4-bromo-2-fluoro-N-[3-(2H-1,2,3,4-tetrazol-5-yl)phenyl]benzene-1-sulfonamide # PATENT #
New compounds # WO-2012035171-A2
Kancera_Example-058 # 3-(5-{[3-(2H-1,2,3,4-tetrazol-5-yl)phenyl]sulfamoyl}thiophen-2-yl)benzamide # PATENT # New
compounds # WO-2012035171-A2
Kancera_Example-059 # 5-(5-chloro-1,2,4-thiadiazol-3-yl)-N-[3-(2H-1,2,3,4-tetrazol-5-yl)phenyl]thiophene-2-sulfonamide
# PATENT # New compounds # WO-2012035171-A2
Kancera_Example-060 # 5-phenyl-N-[3-(2H-1,2,3,4-tetrazol-5-yl)phenyl]thiophene-2-sulfonamide # PATENT # New
compounds # WO-2012035171-A2
Kancera_Example-061 # 3-{5-[2-(methylsulfanyl)pyrimidin-4-yl]thiophene-2-sulfonamido}benzoic acid # PATENT # New
compounds # WO-2012035171-A2
Kancera_Example-062 # 3-[5-(2-methyl-1,3-thiazol-4-yl)thiophene-2-sulfonamido]benzoic acid # PATENT # New
compounds # WO-2012035171-A2
Kancera_Example-063 # N-[3-(2H-1,2,3,4-tetrazol-5-yl)phenyl]-5-[5-(trifluoromethyl)-1,2-oxazol-3-yl]thiophene-2-
sulfonamide # PATENT # New compounds # WO-2012035171-A2
Kancera_Example-064 # N-[3-(2H-1,2,3,4-tetrazol-5-yl)phenyl]-1-benzofuran-2-sulfonamide# PATENT # New
compounds # WO-2012035171-A2
Kancera_Example-065 # 5-(1,2-oxazol-3-yl)-N-[3-(2H-1,2,3,4-tetrazol-5-yl)phenyl]thiophene-2-sulfonamide # PATENT #
New compounds # WO-2012035171-A2
Kancera_Example-066 # 2,4,6-trichloro-N-[3-(2H-1,2,3,4-tetrazol-5-yl)phenyl]benzene-1-sulfonamide # PATENT # New
compounds # WO-2012035171-A2
Kancera_Example-067 # 2,3-dichloro-N-[3-(2H-1,2,3,4-tetrazol-5-yl)phenyl]benzene-1-sulfonamide # PATENT # New
compounds # WO-2012035171-A2
Kancera_Example-068 # 2,5-dichloro-N-[3-(2H-1,2,3,4-tetrazol-5-yl)phenyl]benzene-1-sulfonamide # PATENT # New
compounds # WO-2012035171-A2
Kancera_Example-069 # 3-chloro-2-methyl-N-[3-(2H-1,2,3,4-tetrazol-5-yl)phenyl]benzene-1-sulfonamide # PATENT #
New compounds # WO-2012035171-A2
Kancera_Example-070 # 2,4-dichloro-N-[3-(2H-1,2,3,4-tetrazol-5-yl)phenyl]benzene-1-sulfonamide # PATENT # New
compounds # WO-2012035171-A2
Kancera_Example-071 # 2,4,5-trichloro-N-[3-(2H-1,2,3,4-tetrazol-5-yl)phenyl]benzene-1-sulfonamide # PATENT # New
compounds # WO-2012035171-A2
Kancera_Example-072 # 2,4-difluoro-N-[3-(2H-1,2,3,4-tetrazol-5-yl)phenyl]benzene-1-sulfonamide # PATENT # New
compounds # WO-2012035171-A2
Kancera_Example-073 # 7-chloro-N-[3-(2H-1,2,3,4-tetrazol-5-yl)phenyl]-2,1,3-benzoxadiazole-4-sulfonamide # PATENT
# New compounds # WO-2012035171-A2
Kancera_Example-074 # methyl 3-(5-chloro-3-methyl-1-benzothiophene-2-sulfonamido)benzoate # PATENT # New
compounds # WO-2012035171-A2
Kancera_Example-075 # 3-(3-methyl-1-benzothiophene-2-sulfonamido)benzoic acid # PATENT # New compounds #
WO-2012035171-A2
Kancera_Example-076 # 3-(5-fluoro-3-methyl-1-benzothiophene-2-sulfonamido)benzoic acid # PATENT # New
compounds # WO-2012035171-A2
Kancera_Example-077 # 3-(5-methoxy-3-methyl-1-benzothiophene-2-sulfonamido)benzoic acid # PATENT # New
compounds # WO-2012035171-A2
Kancera_Example-078 # 3-[5-(pyridin-2-yl)thiophene-2-sulfonamido]benzoic acid # PATENT # New compounds # WO-
2012035171-A2
Kancera_Example-079 # 3-[5-(1,2-oxazol-3-yl)thiophene-2-sulfonamido]benzoic acid # PATENT # New compounds #
WO-2012035171-A2
Kancera_Example-080 # 3-{[1,1′-biphenyl]-3-sulfonamido}benzoic acid # PATENT # New compounds # WO-
2012035171-A2
Kancera_Example-081 # 2-chloro-4-fluoro-N-[3-(2H-1,2,3,4-tetrazol-5-yl)phenyl]benzene-1-sulfonamide # PATENT #
New compounds # WO-2012035171-A2
Kancera_Example-082 # N-[3-(2H-1,2,3,4-tetrazol-5-yl)phenyl]-1-benzothiophene-2-sulfonamide # PATENT # New
compounds # WO-2012035171-A2
Kancera_Example-083 # 4-(1,3-oxazol-5-yl)-N-[3-(2H-1,2,3,4-tetrazol-5-yl)phenyl]benzene-1-sulfonamide # PATENT #
New compounds # WO-2012035171-A2
Kancera_Example-084 # 3-(1-benzothiophene-2-sulfonamido)benzoic acid # PATENT # New compounds # WO-
2012035171-A2
Kancera_Example-085 # 4′-methoxy-N-[3-(2H-1,2,3,4-tetrazol-5-yl)phenyl]-[1,1′-biphenyl]-4-sulfonamide # PATENT #
New compounds # WO-2012035171-A2
Kancera_Example-086 # 3′,4′-dichloro-N-[3-(2H-1,2,3,4-tetrazol-5-yl)phenyl]-[1,1′-biphenyl]-4-sulfonamide # PATENT #
New compounds # WO-2012035171-A2
Kancera_Example-087 # 5-(1,2-oxazol-5-yl)-N-[3-(2H-1,2,3,4-tetrazol-5-yl)phenyl]thiophene-2-sulfonamide # PATENT #
New compounds # WO-2012035171-A2
Kancera_Example-088 # methyl 3-[5-(2-methyl-1,3-thiazol-4-yl)thiophene-2-sulfonamido]benzoate # PATENT # New
compounds # WO-2012035171-A2
Kancera_Example-089 # 3-(5-methyl-1-benzothiophene-2-sulfonamido)benzoic acid # PATENT # New compounds #
WO-2012035171-A2
Kancera_Example-090 # 4′-chloro-N-[3-(2H-1,2,3,4-tetrazol-5-yl)phenyl]-[1,1′-biphenyl]-3-sulfonamide # PATENT # New
compounds # WO-2012035171-A2
Kancera_Example-091 # 3′,4′-dichloro-N-[3-(2H-1,2,3,4-tetrazol-5-yl)phenyl]-[1,1′-biphenyl]-3-sulfonamide # PATENT #
New compounds # WO-2012035171-A2
Kancera_Example-092 # methyl 3-[3-methyl-5-(propan-2-yl)-1-benzothiophene-2-sulfonamido]benzoate # PATENT #
New compounds # WO-2012035171-A2
Kancera_Example-093 # 3-{4′-chloro-[1,1′-biphenyl]-3-sulfonamido}benzoic acid # PATENT # New compounds # WO-
2012035171-A2
Kancera_Example-094 # 3-{4′-fluoro-[1,1′-biphenyl]-3-sulfonamido}benzoic acid # PATENT # New compounds # WO-
2012035171-A2
Kancera_Example-095 # 3-{4′-methoxy-[1,1′-biphenyl]-3-sulfonamido}benzoic acid # PATENT # New compounds # WO-
2012035171-A2
Kancera_Example-096 # 3-{3′,4′-dichloro-[1,1′-biphenyl]-3-sulfonamido}benzoic acid # PATENT # New compounds #
WO-2012035171-A2
Kancera_Example-097 # 5-(3-methoxyphenyl)-N-[3-(2H-1,2,3,4-tetrazol-5-yl)phenyl]thiophene-2-sulfonamide # PATENT
# New compounds # WO-2012035171-A2
Kancera_Example-098 # 5-(3,4-dichlorophenyl)-N-[3-(2H-1,2,3,4-tetrazol-5-yl)phenyl]thiophene-2-sulfonamide #
PATENT # New compounds # WO-2012035171-A2
Kancera_Example-099 # N-[3-(2H-1,2,3,4-tetrazol-5-yl)phenyl]-5-[3-(trifluoromethyl)phenyl]thiophene-2-sulfonamide #
PATENT # New compounds # WO-2012035171-A2
Kancera_Example-100 # 5-(2-methylphenyl)-N-[3-(2H-1,2,3,4-tetrazol-5-yl)phenyl]thiophene-2-sulfonamide # PATENT #
New compounds # WO-2012035171-A2
Kancera_Example-101 # 5-(2,4-difluorophenyl)-N-[3-(2H-1,2,3,4-tetrazol-5-yl)phenyl]thiophene-2-sulfonamide # PATENT
# New compounds # WO-2012035171-A2
Kancera_Example-102 # 5-(3-chloro-4-fluorophenyl)-N-[3-(2H-1,2,3,4-tetrazol-5-yl)phenyl]thiophene-2-sulfonamide #
PATENT # New compounds # WO-2012035171-A2
Kancera_Example-103 # 5-(3-chlorophenyl)-N-[3-(2H-1,2,3,4-tetrazol-5-yl)phenyl]thiophene-2-sulfonamide # PATENT #
New compounds # WO-2012035171-A2
Kancera_Example-104 # 5-(pyridin-4-yl)-N-[3-(2H-1,2,3,4-tetrazol-5-yl)phenyl]thiophene-2-sulfonamide # PATENT # New
compounds # WO-2012035171-A2
Kancera_Example-105 # 3-[3-methyl-5-(morpholin-4-yl)-1-benzothiophene-2-sulfonamido]benzoic acid # PATENT # New
compounds # WO-2012035171-A2
Kancera_Example-106 # 2-(1H-pyrrol-1-yl)ethyl 3-(5-chloro-3-methyl-1-benzothiophene-2-sulfonamido)benzoate #
PATENT # New compounds # WO-2012035171-A2
Kancera_Example-107 # ethyl 3-(5-chloro-3-methyl-1-benzothiophene-2-sulfonamido)benzoate # PATENT # New
compounds # WO-2012035171-A2
Kancera_Example-108 # propan-2-yl 3-(5-chloro-3-methyl-1-benzothiophene-2-sulfonamido)benzoate # PATENT # New
compounds # WO-2012035171-A2
Kancera_Example-109 # 2-methoxyethyl 3-(5-chloro-3-methyl-1-benzothiophene-2-sulfonamido)benzoate # PATENT #
New compounds # WO-2012035171-A2
Kancera_Example-110 # butyl 3-(5-chloro-3-methyl-1-benzothiophene-2-sulfonamido)benzoate # PATENT # New
compounds # WO-2012035171-A2
Kancera_Example-111 # benzyl 3-(5-chloro-3-methyl-1-benzothiophene-2-sulfonamido)benzoate # PATENT # New
compounds # WO-2012035171-A2
Kancera_Example-112 # propyl 3-(5-chloro-3-methyl-1-benzothiophene-2-sulfonamido)benzoate # PATENT # New
compounds # WO-2012035171-A2
Kancera_Example-113 # pentyl 3-(5-chloro-3-methyl-1-benzothiophene-2-sulfonamido)benzoate # PATENT # New
compounds # WO-2012035171-A2
Kancera_Example-114 # hexyl 3-(5-chloro-3-methyl-1-benzothiophene-2-sulfonamido)benzoate # PATENT # New
compounds # WO-2012035171-A2
Kancera_Example-115 # phenyl 3-(5-chloro-3-methyl-1-benzothiophene-2-sulfonamido)benzoate # PATENT # New
compounds # WO-2012035171-A2
Kancera_Example-116 # oxolan-3-yl 3-(5-chloro-3-methyl-1-benzothiophene-2-sulfonamido)benzoate # PATENT # New
compounds # WO-2012035171-A2
Kancera_Example-117 # (oxolan-3-yl)methyl 3-(5-chloro-3-methyl-1-benzothiophene-2-sulfonamido)benzoate # PATENT
# New compounds # WO-2012035171-A2
Kancera_Example-118 # 3-(dimethylamino)propyl 3-(5-chloro-3-methyl-1-benzothiophene-2-sulfonamido)benzoate #
PATENT # New compounds # WO-2012035171-A2
Kancera_Example-119 # methyl 2-(5-{3-[3-methyl-5-(propan-2-yl)-1-benzothiophene-2-sulfonamido]phenyl}-2H-1,2,3,4-
tetrazol-2-yl)acetate # PATENT # New compounds # WO-2012035171-A2
Kancera_Example-120 # methyl 3-(5-bromo-3-methyl-1-benzothiophene-2-sulfonamido)benzoate # PATENT # New
compounds # WO-2012035171-A2
Kancera_Example-121 # methyl 3-(7-methoxy-3-methyl-1-benzothiophene-2-sulfonamido)benzoate # PATENT # New
compounds # WO-2012035171-A2
Kancera_Example-122 # methyl 3-(7-chloro-3-methyl-1-benzothiophene-2-sulfonamido)benzoate # PATENT # New
compounds # WO-2012035171-A2
Kancera_Example-123 # methyl 3-[3-methyl-5-(propan-2-yl)-1-benzofuran-2-sulfonamido]benzoate # PATENT # New
compounds # WO-2012035171-A2
Kancera_Example-124 # methyl 2-[3-methyl-5-(propan-2-yl)-1-benzothiophene-2-sulfonamido]-1,3-thiazole-5-carboxylate
# PATENT # New compounds # WO-2012035171-A2
Kancera_Example-125 # ethyl 4-methyl-2-[3-methyl-5-(propan-2-yl)-1-benzothiophene-2-sulfonamido]-1,3-thiazole-5-
carboxylate # PATENT # New compounds # WO-2012035171-A2
Kancera_Example-126 # ethyl 2-(5-chloro-3-methyl-1-benzothiophene-2-sulfonamido)-4-methyl-1,3-thiazole-5-
carboxylate # PATENT # New compounds # WO-2012035171-A2
Kancera_Example-127 # ethyl 2-[5-chloro-4-(2,5-difluorophenyl)thiophene-2-sulfonamido]-4-methyl-1,3-thiazole-5-
carboxylate # PATENT # New compounds # WO-2012035171-A2
Kancera_Example-128 # 2-(5-chloro-3-methyl-1-benzothiophene-2-sulfonamido)-4-methyl-1,3-thiazole-5-carboxylic acid
# PATENT # New compounds # WO-2012035171-A2
Kancera_Example-129 # 2-[5-chloro-4-(2,5-difluorophenyl)thiophene-2-sulfonamido]-4-methyl-1,3-thiazole-5-carboxylic
acid # PATENT # New compounds # WO-2012035171-A2
Kancera_Example-130 # 2-(5-chloro-3-methyl-1-benzothiophene-2-sulfonamido)-1,3-thiazole-5-carboxylic acid #
PATENT # New compounds # WO-2012035171-A2
Kancera_Example-131 # 2-[5-(3,5-difluorophenyl)thiophene-2-sulfonamido]-5-methyl-1,3-thiazole-4-carboxylic acid #
PATENT # New compounds # WO-2012035171-A2
Kancera_Example-132 # 2-[5-chloro-4-(2,5-difluorophenyl)thiophene-2-sulfonamido]-5-methyl-1,3-thiazole-4-carboxylic
acid # PATENT # New compounds # WO-2012035171-A2
Kancera_Example-133 # 5-methyl-2-[3-methyl-5-(propan-2-yl)-1-benzothiophene-2-sulfonamido]-1,3-thiazole-4-
carboxylic acid # PATENT # New compounds # WO-2012035171-A2
Kancera_Example-134 # 3-(5-chloro-3-methyl-1-benzothiophene-2-sulfonamido)-2-hydroxybenzoic acid # PATENT #
New compounds # WO-2012035171-A2
Kancera_Example-135 # 3-[5-chloro-4-(2,5-difluorophenyl)thiophene-2-sulfonamido]-2-hydroxybenzoic acid # PATENT #
New compounds # WO-2012035171-A2
Kancera_Example-136 # 3-[5-chloro-4-(2,3-dihydro-1-benzofuran-5-yl)thiophene-2-sulfonamido]-2-hydroxybenzoic acid #
PATENT # New compounds # WO-2012035171-A2
Kancera_Example-137 # 3-[5-chloro-4-(2-hydroxyphenyl)thiophene-2-sulfonamido]-2-hydroxybenzoic acid # PATENT #
New compounds # WO-2012035171-A2
Kancera_Example-138 # 5-(5-chloro-3-methyl-1-benzothiophene-2-sulfonamido)-2-hydroxybenzoic acid # PATENT #
New compounds # WO-2012035171-A2
Kancera_Example-139 # 5-{4′-chloro-[1,1′-biphenyl]-3-sulfonamido}-2-hydroxybenzoic acid # PATENT # New compounds
# WO-2012035171-A2
Kancera_Example-140 # methyl 5-(5-chloro-3-methyl-1-benzothiophene-2-sulfonamido)-2-hydroxybenzoate # PATENT #
New compounds # WO-2012035171-A2
Kancera_Example-142 # 5-(benzenesulfonyl)-N-[3-(2H-1,2,3,4-tetrazol-5-yl)phenyl]thiophene-2-sulfonamide # PATENT #
New compounds # WO-2012035171-A2
Kancera_Example-143 # 2,2-dimethyl-N-[3-(2H-1,2,3,4-tetrazol-5-yl)phenyl]-3,4-dihydro-2H-1-benzopyran-6-sulfonamide
# PATENT # New compounds # WO-2012035171-A2
Kancera_Example2-001 # 4-{[1,1′-biphenyl]-3-sulfonamido}-2-hydroxybenzoic acid # PATENT # Bisarylsulfonamides
useful as kinase inhibitors in the treatment of inflammation and cancer # WO-2011161201-A1
Kancera_Example2-002 # 4-(3-bromobenzenesulfonamido)-2-hydroxybenzoic acid # PATENT # Bisarylsulfonamides
useful as kinase inhibitors in the treatment of inflammation and cancer # WO-2011161201-A1
Kancera_Example2-003 # 4-[3-(5-acetylthiophen-2-yl)benzenesulfonamido]-2-hydroxybenzoic acid # PATENT #
Bisarylsulfonamides useful as kinase inhibitors in the treatment of inflammation and cancer # WO-2011161201-A1
Kancera_Example2-004 # 2-hydroxy-4-{4′-hydroxy-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-005 # 4-{3-[(E)-2-(4-fluorophenyl)ethenyl]benzenesulfonamido}-2-hydroxybenzoic acid # PATENT #
Bisarylsulfonamides useful as kinase inhibitors in the treatment of inflammation and cancer # WO-2011161201-A1
Kancera_Example2-006 # 4-{3′-amino-4′-methoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-007 # 2-hydroxy-4-[3-(pyridin-3-yl)benzenesulfonamido]benzoic acid # PATENT #
Bisarylsulfonamides useful as kinase inhibitors in the treatment of inflammation and cancer # WO-2011161201-A1
Kancera_Example2-008 # 4-[4′-(dimethylamino)-[1,1′-biphenyl]-3-sulfonamido]-2-hydroxybenzoic acid # PATENT #
Bisarylsulfonamides useful as kinase inhibitors in the treatment of inflammation and cancer # WO-2011161201-A1
Kancera_Example2-009 # 2-hydroxy-4-[5-(trifluoromethyl)-[1,1′-biphenyl]-3-sulfonamido]benzoic acid # PATENT #
Bisarylsulfonamides useful as kinase inhibitors in the treatment of inflammation and cancer # WO-2011161201-A1
Kancera_Example2-010 # 4-{4,6-difluoro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-011 # 2-hydroxy-4-{6-methoxy-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-012 # 4-(5-chloro-4-phenylthiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-013 # 2-hydroxy-4-[2′-(hydroxymethyl)-[1,1′-biphenyl]-3-sulfonamido]benzoic acid # PATENT #
Bisarylsulfonamides useful as kinase inhibitors in the treatment of inflammation and cancer # WO-2011161201-A1
Kancera_Example2-014 # 4-{3′-fluoro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-015 # 4-{2′,6′-difluoro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-016 # 2-hydroxy-4-{3′-[(propan-2-yloxy)carbonyl]-[1,1′-biphenyl]-3-sulfonamido}benzoic acid #
PATENT # Bisarylsulfonamides useful as kinase inhibitors in the treatment of inflammation and cancer # WO-
2011161201-A1
Kancera_Example2-017 # 4-[3-(2,3-dihydro-1-benzofuran-5-yl)benzenesulfonamido]-2-hydroxybenzoic acid # PATENT #
Bisarylsulfonamides useful as kinase inhibitors in the treatment of inflammation and cancer # WO-2011161201-A1
Kancera_Example2-018 # 4-{3′-fluoro-4′-hydroxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-019 # 2-hydroxy-4-[3-(quinolin-6-yl)benzenesulfonamido]benzoic acid # PATENT #
Bisarylsulfonamides useful as kinase inhibitors in the treatment of inflammation and cancer # WO-2011161201-A1
Kancera_Example2-020 # 4-{3′-amino-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-021 # 2-hydroxy-4-[3-(2-methyl-1,3-thiazol-4-yl)benzenesulfonamido]benzoic acid # PATENT #
Bisarylsulfonamides useful as kinase inhibitors in the treatment of inflammation and cancer # WO-2011161201-A1
Kancera_Example2-022 # 4-(5-chlorothiophene-2-sulfonamido)-2-hydroxybenzoic acid # PATENT # Bisarylsulfonamides
useful as kinase inhibitors in the treatment of inflammation and cancer # WO-2011161201-A1
Kancera_Example2-023 # 4-[5-chloro-4-(2,3-dihydro-1-benzofuran-5-yl)thiophene-2-sulfonamido]-2-hydroxybenzoic acid
# PATENT # Bisarylsulfonamides useful as kinase inhibitors in the treatment of inflammation and cancer # WO-
2011161201-A1
Kancera_Example2-024 # 4-[5-chloro-4-(3-fluoro-4-hydroxyphenyl)thiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-025 # 4-[5-chloro-4-(quinolin-6-yl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-026 # 4-[4-(2H-1,3-benzodioxol-5-yl)-5-chlorothiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-027 # 4-[5-chloro-4-(4-hydroxy-3,5-dimethylphenyl)thiophene-2-sulfonamido]-2-hydroxybenzoic acid
# PATENT # Bisarylsulfonamides useful as kinase inhibitors in the treatment of inflammation and cancer # WO-
2011161201-A1
Kancera_Example2-028 # 4-[5-chloro-4-(2,4-difluorophenyl)thiophene-2-sulfonamido]-2-hydroxybenzoic acid # PATENT
# Bisarylsulfonamides useful as kinase inhibitors in the treatment of inflammation and cancer # WO-2011161201-A1
Kancera_Example2-029 # 4-[4-(3-acetylphenyl)-5-chlorothiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-030 # 4-{5-chloro-4-[2-(hydroxymethyl)phenyl]thiophene-2-sulfonamido}-2-hydroxybenzoic acid #
PATENT # Bisarylsulfonamides useful as kinase inhibitors in the treatment of inflammation and cancer # WO-
2011161201-A1
Kancera_Example2-031 # 4-[5-chloro-4-(3-fluorophenyl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-032 # 4-(5-chloro-4-{3-[(propan-2-yloxy)carbonyl]phenyl}thiophene-2-sulfonamido)-2-hydroxybenzoic
acid # PATENT # Bisarylsulfonamides useful as kinase inhibitors in the treatment of inflammation and cancer # WO-
2011161201-A1
Kancera_Example2-033 # 4-[5-chloro-4-(3,5-difluorophenyl)thiophene-2-sulfonamido]-2-hydroxybenzoic acid # PATENT
# Bisarylsulfonamides useful as kinase inhibitors in the treatment of inflammation and cancer # WO-2011161201-A1
Kancera_Example2-034 # 4-[5-chloro-4-(6-ethoxypyridin-3-yl)thiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-035 # 4-[4-(3-aminophenyl)-5-chlorothiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-036 # 4-[5-chloro-4-(4-methoxyphenyl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-037 # 4-[4-(4-aminophenyl)-5-chlorothiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-038 # 4-[5-chloro-4-(4-hydroxyphenyl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-039 # 4-{5-chloro-4-[3-(hydroxymethyl)phenyl]thiophene-2-sulfonamido}-2-hydroxybenzoic acid #
PATENT # Bisarylsulfonamides useful as kinase inhibitors in the treatment of inflammation and cancer # WO-
2011161201-A1
Kancera_Example2-040 # 4-{5-chloro-4-[4-(hydroxymethyl)phenyl]thiophene-2-sulfonamido}-2-hydroxybenzoic acid #
PATENT # Bisarylsulfonamides useful as kinase inhibitors in the treatment of inflammation and cancer # WO-
2011161201-A1
Kancera_Example2-041 # 4-[4-(3-amino-4-methoxyphenyl)-5-chlorothiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-042 # 4-[5-chloro-4-(4-methanesulfonamidophenyl)thiophene-2-sulfonamido]-2-hydroxybenzoic acid
# PATENT # Bisarylsulfonamides useful as kinase inhibitors in the treatment of inflammation and cancer # WO-
2011161201-A1
Kancera_Example2-043 # 4-(7-chloro-3-methyl-1-benzothiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-044 # 4-(5-chloro-3-methyl-1-benzothiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-045 # 2-hydroxy-4-[3-(piperidin-1-yl)benzenesulfonamido]benzoic acid # PATENT #
Bisarylsulfonamides useful as kinase inhibitors in the treatment of inflammation and cancer # WO-2011161201-A1
Kancera_Example2-046 # 4-(3-acetylbenzenesulfonamido)-2-hydroxybenzoic acid # PATENT # Bisarylsulfonamides
useful as kinase inhibitors in the treatment of inflammation and cancer # WO-2011161201-A1
Kancera_Example2-047 # 4-(3-tert-butylbenzenesulfonamido)-2-hydroxybenzoic acid # PATENT # Bisarylsulfonamides
useful as kinase inhibitors in the treatment of inflammation and cancer # WO-2011161201-A1
Kancera_Example2-048 # 2-hydroxy-4-(4-phenylthiophene-2-sulfonamido)benzoic acid # PATENT # Bisarylsulfonamides
useful as kinase inhibitors in the treatment of inflammation and cancer # WO-2011161201-A1
Kancera_Example2-049 # 2-hydroxy-4-[3-(piperidine-1-carbonyl)benzenesulfonamido]benzoic acid # PATENT #
Bisarylsulfonamides useful as kinase inhibitors in the treatment of inflammation and cancer # WO-2011161201-A1
Kancera_Example2-050 # 2-hydroxy-4-[3-(methylcarbamoyl)benzenesulfonamido]benzoic acid # PATENT #
Bisarylsulfonamides useful as kinase inhibitors in the treatment of inflammation and cancer # WO-2011161201-A1
Kancera_Example2-051 # 2-hydroxy-4-{4′-methanesulfonyl-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-052 # 4-{3′-ethoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-053 # 4-{3′-acetamido-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-054 # 4-{3′,4′-dichloro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-055 # 4-{3′-carbamoyl-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-056 # 4-{3′-cyano-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-057 # 2-hydroxy-4-{4′-nitro-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-058 # 2-hydroxy-4-[3′-(trifluoromethyl)-[1,1′-biphenyl]-3-sulfonamido]benzoic acid # PATENT #
Bisarylsulfonamides useful as kinase inhibitors in the treatment of inflammation and cancer # WO-2011161201-A1
Kancera_Example2-059 # 2-hydroxy-4-[4′-(methylsulfanyl)-[1,1′-biphenyl]-3-sulfonamido]benzoic acid # PATENT #
Bisarylsulfonamides useful as kinase inhibitors in the treatment of inflammation and cancer # WO-2011161201-A1
Kancera_Example2-060 # 2-hydroxy-4-[4′-(trifluoromethoxy)-[1,1′-biphenyl]-3-sulfonamido]benzoic acid # PATENT #
Bisarylsulfonamides useful as kinase inhibitors in the treatment of inflammation and cancer # WO-2011161201-A1
Kancera_Example2-061 # 4-{2′-acetyl-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-062 # 2-hydroxy-4-{4′-phenoxy-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-063 # 2-hydroxy-4-{4′-hydroxy-3′-methoxy-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-064 # 2-hydroxy-4-(3-methanesulfonylbenzenesulfonamido)benzoic acid # PATENT #
Bisarylsulfonamides useful as kinase inhibitors in the treatment of inflammation and cancer # WO-2011161201-A1
Kancera_Example2-065 # 4-[3-(1-benzofuran-2-yl)benzenesulfonamido]-2-hydroxybenzoic acid # PATENT #
Bisarylsulfonamides useful as kinase inhibitors in the treatment of inflammation and cancer # WO-2011161201-A1
Kancera_Example2-066 # 2-hydroxy-4-{4′-[(methoxycarbonyl)amino]-[1,1′-biphenyl]-3-sulfonamido}benzoic acid #
PATENT # Bisarylsulfonamides useful as kinase inhibitors in the treatment of inflammation and cancer # WO-
2011161201-A1
Kancera_Example2-067 # 4-(5-fluoro-2-methylbenzenesulfonamido)-2-hydroxybenzoic acid # PATENT #
Bisarylsulfonamides useful as kinase inhibitors in the treatment of inflammation and cancer # WO-2011161201-A1
Kancera_Example2-068 # 4-(2-bromo-4-iodobenzenesulfonamido)-2-hydroxybenzoic acid # PATENT #
Bisarylsulfonamides useful as kinase inhibitors in the treatment of inflammation and cancer # WO-2011161201-A1
Kancera_Example2-069 # 2-hydroxy-4-(2,4,5-trichlorobenzenesulfonamido)benzoic acid # PATENT #
Bisarylsulfonamides useful as kinase inhibitors in the treatment of inflammation and cancer # WO-2011161201-A1
Kancera_Example2-070 # 2-hydroxy-4-[4-(1,3-oxazol-5-yl)benzenesulfonamido]benzoic acid # PATENT #
Bisarylsulfonamides useful as kinase inhibitors in the treatment of inflammation and cancer # WO-2011161201-A1
Kancera_Example2-071 # 4-(2,1,3-benzothiadiazole-4-sulfonamido)-2-hydroxybenzoic acid # PATENT #
Bisarylsulfonamides useful as kinase inhibitors in the treatment of inflammation and cancer # WO-2011161201-A1
Kancera_Example2-072 # 4-(2,1,3-benzoxadiazole-4-sulfonamido)-2-hydroxybenzoic acid # PATENT #
Bisarylsulfonamides useful as kinase inhibitors in the treatment of inflammation and cancer # WO-2011161201-A1
Kancera_Example2-073 # 4-{4′-chloro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-074 # 2-hydroxy-4-[4′-(trifluoromethyl)-[1,1′-biphenyl]-3-sulfonamido]benzoic acid # PATENT #
Bisarylsulfonamides useful as kinase inhibitors in the treatment of inflammation and cancer # WO-2011161201-A1
Kancera_Example2-075 # 4-{4′-fluoro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-076 # 4-{3′,5′-dichloro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-077 # 2-hydroxy-4-{4′-methoxy-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-078 # 2-hydroxy-4-{4′-methyl-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-079 # 2-hydroxy-4-[3-(trifluoromethyl)benzenesulfonamido]benzoic acid # PATENT #
Bisarylsulfonamides useful as kinase inhibitors in the treatment of inflammation and cancer # WO-2011161201-A1
Kancera_Example2-080 # 4-(1-benzothiophene-2-sulfonamido)-2-hydroxybenzoic acid # PATENT # Bisarylsulfonamides
useful as kinase inhibitors in the treatment of inflammation and cancer # WO-2011161201-A1
Kancera_Example2-081 # 2-hydroxy-4-(5-methyl-1-benzothiophene-2-sulfonamido)benzoic acid # PATENT #
Bisarylsulfonamides useful as kinase inhibitors in the treatment of inflammation and cancer # WO-2011161201-A1
Kancera_Example2-082 # 2-hydroxy-4-(7-methoxy-3-methyl-1-benzothiophene-2-sulfonamido)benzoic acid # PATENT #
Bisarylsulfonamides useful as kinase inhibitors in the treatment of inflammation and cancer # WO-2011161201-A1
Kancera_Example2-083 # 2-hydroxy-4-(5-methoxy-3-methyl-1-benzothiophene-2-sulfonamido)benzoic acid # PATENT #
Bisarylsulfonamides useful as kinase inhibitors in the treatment of inflammation and cancer # WO-2011161201-A1
Kancera_Example2-084 # 2-hydroxy-4-[3-methyl-5-(propan-2-yl)-1-benzofuran-2-sulfonamido]benzoic acid # PATENT #
Bisarylsulfonamides useful as kinase inhibitors in the treatment of inflammation and cancer # WO-2011161201-A1
Kancera_Example2-085 # 4-(5-fluoro-3-methyl-1-benzothiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-086 # 4-[3-(2H-1,3-benzodioxol-5-yl)benzenesulfonamido]-2-hydroxybenzoic acid # PATENT #
Bisarylsulfonamides useful as kinase inhibitors in the treatment of inflammation and cancer # WO-2011161201-A1
Kancera_Example2-087 # 4-{2′,4′-difluoro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-088 # 2-hydroxy-4-{2′-nitro-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-089 # 2-hydroxy-4-{4′-hydroxy-3′,5′-dimethyl-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-090 # 4-{4′-butyl-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-091 # 4-[4′-(ethanesulfonyl)-[1,1′-biphenyl]-3-sulfonamido]-2-hydroxybenzoic acid # PATENT #
Bisarylsulfonamides useful as kinase inhibitors in the treatment of inflammation and cancer # WO-2011161201-A1
Kancera_Example2-092 # 2-hydroxy-4-{4′-methoxy-3′-methyl-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-093 # 2-hydroxy-4-{3′-hydroxy-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-094 # 2-hydroxy-4-{3′-methanesulfonyl-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-095 # 4-[4′-(dimethylcarbamoyl)-[1,1′-biphenyl]-3-sulfonamido]-2-hydroxybenzoic acid # PATENT #
Bisarylsulfonamides useful as kinase inhibitors in the treatment of inflammation and cancer # WO-2011161201-A1
Kancera_Example2-096 # 4-{4′-ethyl-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-097 # 4-{4′-[bis(propan-2-yl)carbamoyl]-[1,1′-biphenyl]-3-sulfonamido}-2-hydroxybenzoic acid #
PATENT # Bisarylsulfonamides useful as kinase inhibitors in the treatment of inflammation and cancer # WO-
2011161201-A1
Kancera_Example2-098 # 4-{4′-acetyl-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-099 # 4-{2′,3′-dimethoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-100 # 4-{4′-fluoro-2′-methoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-101 # 2-hydroxy-4-{2′,3′,6′-trifluoro-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-102 # 4-[4′-(2-carboxyethyl)-[1,1′-biphenyl]-3-sulfonamido]-2-hydroxybenzoic acid # PATENT #
Bisarylsulfonamides useful as kinase inhibitors in the treatment of inflammation and cancer # WO-2011161201-A1
Kancera_Example2-103 # 2-hydroxy-4-{3′-methyl-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-104 # 4-{3′,5′-difluoro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-105 # 2-hydroxy-4-{4′-methoxy-3′,5′-dimethyl-[1,1′-biphenyl]-3-sulfonamido}benzoic acid # PATENT
# Bisarylsulfonamides useful as kinase inhibitors in the treatment of inflammation and cancer # WO-2011161201-A1
Kancera_Example2-106 # 2-hydroxy-4-{2′-methyl-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-107 # 2-hydroxy-4-[3-(2-propoxypyridin-3-yl)benzenesulfonamido]benzoic acid # PATENT #
Bisarylsulfonamides useful as kinase inhibitors in the treatment of inflammation and cancer # WO-2011161201-A1
Kancera_Example2-108 # 4-[3-(6-ethoxypyridin-3-yl)benzenesulfonamido]-2-hydroxybenzoic acid # PATENT #
Bisarylsulfonamides useful as kinase inhibitors in the treatment of inflammation and cancer # WO-2011161201-A1
Kancera_Example2-109 # 2-hydroxy-4-[4′-(propan-2-yloxy)-[1,1′-biphenyl]-3-sulfonamido]benzoic acid # PATENT #
Bisarylsulfonamides useful as kinase inhibitors in the treatment of inflammation and cancer # WO-2011161201-A1
Kancera_Example2-110 # 4-{4′-butoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-111 # 4-{3′,4′-dimethoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-112 # 2-hydroxy-4-[3-(6-methoxypyridin-3-yl)benzenesulfonamido]benzoic acid # PATENT #
Bisarylsulfonamides useful as kinase inhibitors in the treatment of inflammation and cancer # WO-2011161201-A1
Kancera_Example2-113 # 2-hydroxy-4-[3-(morpholin-4-yl)benzenesulfonamido]benzoic acid # PATENT #
Bisarylsulfonamides useful as kinase inhibitors in the treatment of inflammation and cancer # WO-2011161201-A1
Kancera_Example2-114 # 2-hydroxy-4-(5-phenyl-2,3-dihydro-1-benzofuran-7-sulfonamido)benzoic acid # PATENT #
Bisarylsulfonamides useful as kinase inhibitors in the treatment of inflammation and cancer # WO-2011161201-A1
Kancera_Example2-115 # 4-(4-bromo-5-chlorothiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-116 # 4-(5-bromo-6-chloropyridine-3-sulfonamido)-2-hydroxybenzoic acid # PATENT #
Bisarylsulfonamides useful as kinase inhibitors in the treatment of inflammation and cancer # WO-2011161201-A1
Kancera_Example2-117 # 4-(4,5-dichlorothiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-118 # 4-(3-bromothiophene-2-sulfonamido)-2-hydroxybenzoic acid # PATENT # Bisarylsulfonamides
useful as kinase inhibitors in the treatment of inflammation and cancer # WO-2011161201-A1
Kancera_Example2-119 # 4-(5-bromothiophene-2-sulfonamido)-2-hydroxybenzoic acid # PATENT # Bisarylsulfonamides
useful as kinase inhibitors in the treatment of inflammation and cancer # WO-2011161201-A1
Kancera_Example2-120 # 4-(4-chloro-3-nitrobenzenesulfonamido)-2-hydroxybenzoic acid # PATENT #
Bisarylsulfonamides useful as kinase inhibitors in the treatment of inflammation and cancer # WO-2011161201-A1
Kancera_Example2-121 # 4-(4-bromo-2,5-dichlorothiophene-3-sulfonamido)-2-hydroxybenzoic acid # PATENT #
Bisarylsulfonamides useful as kinase inhibitors in the treatment of inflammation and cancer # WO-2011161201-A1
Kancera_Example2-122 # 4-[3-(difluoromethoxy)benzenesulfonamido]-2-hydroxybenzoic acid # PATENT #
Bisarylsulfonamides useful as kinase inhibitors in the treatment of inflammation and cancer # WO-2011161201-A1
Kancera_Example2-123 # 2-hydroxy-4-(3-methoxybenzenesulfonamido)benzoic acid # PATENT # Bisarylsulfonamides
useful as kinase inhibitors in the treatment of inflammation and cancer # WO-2011161201-A1
Kancera_Example2-124 # 2-hydroxy-4-{5-[(phenylformamido)methyl]thiophene-2-sulfonamido}benzoic acid # PATENT #
Bisarylsulfonamides useful as kinase inhibitors in the treatment of inflammation and cancer # WO-2011161201-A1
Kancera_Example2-125 # 4-(3-chloro-4-methylbenzenesulfonamido)-2-hydroxybenzoic acid # PATENT #
Bisarylsulfonamides useful as kinase inhibitors in the treatment of inflammation and cancer # WO-2011161201-A1
Kancera_Example2-126 # 2-hydroxy-4-(4-methyl-3-nitrobenzenesulfonamido)benzoic acid # PATENT #
Bisarylsulfonamides useful as kinase inhibitors in the treatment of inflammation and cancer # WO-2011161201-A1
Kancera_Example2-127 # 4-(4-bromobenzenesulfonamido)-2-hydroxybenzoic acid # PATENT # Bisarylsulfonamides
useful as kinase inhibitors in the treatment of inflammation and cancer # WO-2011161201-A1
Kancera_Example2-128 # 4-(3-fluorobenzenesulfonamido)-2-hydroxybenzoic acid # PATENT # Bisarylsulfonamides
useful as kinase inhibitors in the treatment of inflammation and cancer # WO-2011161201-A1
Kancera_Example2-129 # 4-(2,5-dichlorothiophene-3-sulfonamido)-2-hydroxybenzoic acid # PATENT #
Bisarylsulfonamides useful as kinase inhibitors in the treatment of inflammation and cancer # WO-2011161201-A1
Kancera_Example2-130 # 2-hydroxy-4-(2,3,4-trichlorobenzenesulfonamido)benzoic acid # PATENT #
Bisarylsulfonamides useful as kinase inhibitors in the treatment of inflammation and cancer # WO-2011161201-A1
Kancera_Example2-131 # 2-hydroxy-4-(4-methylnaphthalene-1-sulfonamido)benzoic acid # PATENT #
Bisarylsulfonamides useful as kinase inhibitors in the treatment of inflammation and cancer # WO-2011161201-A1
Kancera_Example2-132 # 4-(4-fluoronaphthalene-1-sulfonamido)-2-hydroxybenzoic acid # PATENT #
Bisarylsulfonamides useful as kinase inhibitors in the treatment of inflammation and cancer # WO-2011161201-A1
Kancera_Example2-133 # 4-[5-(dimethylamino)naphthalene-1-sulfonamido]-2-hydroxybenzoic acid # PATENT #
Bisarylsulfonamides useful as kinase inhibitors in the treatment of inflammation and cancer # WO-2011161201-A1
Kancera_Example2-135 # 2-hydroxy-4-[3-(pyridin-4-yl)benzenesulfonamido]benzoic acid # PATENT #
Bisarylsulfonamides useful as kinase inhibitors in the treatment of inflammation and cancer # WO-2011161201-A1
Kancera_Example2-136 # 4-{4′-fluoro-3′-methyl-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-137 # 4-{3′-chloro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-138 # 4-{4′-carbamoyl-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-139 # 4-{3′-fluoro-4′-methoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-140 # 4-[6-chloro-5-(4-hydroxyphenyl)pyridine-3-sulfonamido]-2-hydroxybenzoic acid # PATENT #
Bisarylsulfonamides useful as kinase inhibitors in the treatment of inflammation and cancer # WO-2011161201-A1
Kancera_Example2-141 # 4-[6-chloro-5-(3-hydroxyphenyl)pyridine-3-sulfonamido]-2-hydroxybenzoic acid # PATENT #
Bisarylsulfonamides useful as kinase inhibitors in the treatment of inflammation and cancer # WO-2011161201-A1
Kancera_Example2-142 # 4-[5-(3-aminophenyl)-6-chloropyridine-3-sulfonamido]-2-hydroxybenzoic acid # PATENT #
Bisarylsulfonamides useful as kinase inhibitors in the treatment of inflammation and cancer # WO-2011161201-A1
Kancera_Example2-143 # 4-[6-chloro-5-(1H-pyrazol-4-yl)pyridine-3-sulfonamido]-2-hydroxybenzoic acid # PATENT #
Bisarylsulfonamides useful as kinase inhibitors in the treatment of inflammation and cancer # WO-2011161201-A1
Kancera_Example2-144 # 4-[6-chloro-5-(4-fluoro-3-methylphenyl)pyridine-3-sulfonamido]-2-hydroxybenzoic acid #
PATENT # Bisarylsulfonamides useful as kinase inhibitors in the treatment of inflammation and cancer # WO-
2011161201-A1
Kancera_Example2-145 # 4-[6-chloro-5-(3-chlorophenyl)pyridine-3-sulfonamido]-2-hydroxybenzoic acid # PATENT #
Bisarylsulfonamides useful as kinase inhibitors in the treatment of inflammation and cancer # WO-2011161201-A1
Kancera_Example2-146 # 4-[6-chloro-5-(2-fluoro-3-methoxyphenyl)pyridine-3-sulfonamido]-2-hydroxybenzoic acid #
PATENT # Bisarylsulfonamides useful as kinase inhibitors in the treatment of inflammation and cancer # WO-
2011161201-A1
Kancera_Example2-147 # 4-[5-(4-carbamoylphenyl)-6-chloropyridine-3-sulfonamido]-2-hydroxybenzoic acid # PATENT #
Bisarylsulfonamides useful as kinase inhibitors in the treatment of inflammation and cancer # WO-2011161201-A1
Kancera_Example2-148 # 4-[6-chloro-5-(3-fluorophenyl)pyridine-3-sulfonamido]-2-hydroxybenzoic acid # PATENT #
Bisarylsulfonamides useful as kinase inhibitors in the treatment of inflammation and cancer # WO-2011161201-A1
Kancera_Example2-149 # 4-[6-chloro-5-(3-fluoro-4-methoxyphenyl)pyridine-3-sulfonamido]-2-hydroxybenzoic acid #
PATENT # Bisarylsulfonamides useful as kinase inhibitors in the treatment of inflammation and cancer # WO-
2011161201-A1
Kancera_Example2-150 # 4-[6-chloro-5-(quinolin-6-yl)pyridine-3-sulfonamido]-2-hydroxybenzoic acid # PATENT #
Bisarylsulfonamides useful as kinase inhibitors in the treatment of inflammation and cancer # WO-2011161201-A1
Kancera_Example2-151 # 4-[5-chloro-4-(pyridin-3-yl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-152 # 4-[5-chloro-4-(3-hydroxyphenyl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-153 # 4-[5-chloro-4-(4-hydroxy-3-methoxyphenyl)thiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-154 # 4-[5-chloro-4-(3-chlorophenyl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-155 # 4-[4-(4-carbamoylphenyl)-5-chlorothiophene-2-sulfonamido]-2-hydroxybenzoic acid # PATENT
# Bisarylsulfonamides useful as kinase inhibitors in the treatment of inflammation and cancer # WO-2011161201-A1
Kancera_Example2-156 # 4-[5-chloro-4-(3-fluoro-4-methoxyphenyl)thiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-157 # 4-[4-(4-amino-3-methoxyphenyl)-5-chlorothiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-158 # 2-hydroxy-4-[2′-(methoxycarbonyl)-[1,1′-biphenyl]-3-sulfonamido]benzoic acid # PATENT #
Bisarylsulfonamides useful as kinase inhibitors in the treatment of inflammation and cancer # WO-2011161201-A1
Kancera_Example2-159 # 4-{5′-chloro-2′-methoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-160 # 4-{2′,5′-difluoro-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-161 # 2-hydroxy-4-{2′-methoxy-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-162 # 4-{2′-fluoro-3′-methoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-163 # 2-hydroxy-4-{2′-hydroxy-[1,1′-biphenyl]-3-sulfonamido}benzoic acid # PATENT #
Bisarylsulfonamides useful as kinase inhibitors in the treatment of inflammation and cancer # WO-2011161201-A1
Kancera_Example2-164 # 4-{2′-amino-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-165 # 4-{5′-fluoro-2′-methoxy-[1,1′-biphenyl]-3-sulfonamido}-2-hydroxybenzoic acid # PATENT #
Bisarylsulfonamides useful as kinase inhibitors in the treatment of inflammation and cancer # WO-2011161201-A1
Kancera_Example2-166 # 4-[5-chloro-4-(5-chloro-2-methoxyphenyl)thiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-167 # 4-[5-chloro-4-(2,5-difluorophenyl)thiophene-2-sulfonamido]-2-hydroxybenzoic acid # PATENT
# Bisarylsulfonamides useful as kinase inhibitors in the treatment of inflammation and cancer # WO-2011161201-A1
Kancera_Example2-168 # 4-[5-chloro-4-(2-methoxyphenyl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-169 # 4-[5-chloro-4-(2-fluoro-3-methoxyphenyl)thiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-170 # 4-[4-(2-aminophenyl)-5-chlorothiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-171 # 4-[5-chloro-4-(5-fluoro-2-methoxyphenyl)thiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-172 # 4-[5-chloro-4-(2-hydroxyphenyl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-173 # 4-(2,3-dichlorobenzenesulfonamido)-2-hydroxybenzoic acid # PATENT # Bisarylsulfonamides
useful as kinase inhibitors in the treatment of inflammation and cancer # WO-2011161201-A1
Kancera_Example2-174 # 4-(3-chloro-4-fluorobenzenesulfonamido)-2-hydroxybenzoic acid # PATENT #
Bisarylsulfonamides useful as kinase inhibitors in the treatment of inflammation and cancer # WO-2011161201-A1
Kancera_Example2-175 # 4-(4-bromo-2,5-difluorobenzenesulfonamido)-2-hydroxybenzoic acid # PATENT #
Bisarylsulfonamides useful as kinase inhibitors in the treatment of inflammation and cancer # WO-2011161201-A1
Kancera_Example2-176 # 2-hydroxy-4-(3-methylbenzenesulfonamido)benzoic acid # PATENT # Bisarylsulfonamides
useful as kinase inhibitors in the treatment of inflammation and cancer # WO-2011161201-A1
Kancera_Example2-177 # 4-{[1,1′-biphenyl]-4-sulfonamido}-2-hydroxybenzoic acid # PATENT # Bisarylsulfonamides
useful as kinase inhibitors in the treatment of inflammation and cancer # WO-2011161201-A1
Kancera_Example2-178 # 4-(1-benzothiophene-3-sulfonamido)-2-hydroxybenzoic acid # PATENT # Bisarylsulfonamides
useful as kinase inhibitors in the treatment of inflammation and cancer # WO-2011161201-A1
Kancera_Example2-179 # 4-(2,5-dichloro-4-methylthiophene-3-sulfonamido)-2-hydroxybenzoic acid # PATENT #
Bisarylsulfonamides useful as kinase inhibitors in the treatment of inflammation and cancer # WO-2011161201-A1
Kancera_Example2-180 # 2-hydroxy-4-(2,4,5-trichlorothiophene-3-sulfonamido)benzoic acid # PATENT #
Bisarylsulfonamides useful as kinase inhibitors in the treatment of inflammation and cancer # WO-2011161201-A1
Kancera_Example2-181 # 4-(2-chloro-6-methylbenzenesulfonamido)-2-hydroxybenzoic acid # PATENT #
Bisarylsulfonamides useful as kinase inhibitors in the treatment of inflammation and cancer # WO-2011161201-A1
Kancera_Example2-182 # 2-hydroxy-4-[3-(trifluoromethoxy)benzenesulfonamido]benzoic acid # PATENT #
Bisarylsulfonamides useful as kinase inhibitors in the treatment of inflammation and cancer # WO-2011161201-A1
Kancera_Example2-183 # 4-(1-benzofuran-2-sulfonamido)-2-hydroxybenzoic acid # PATENT # Bisarylsulfonamides
useful as kinase inhibitors in the treatment of inflammation and cancer # WO-2011161201-A1
Kancera_Example2-184 # 2-hydroxy-4-[5-methyl-2-(trifluoromethyl)furan-3-sulfonamido]benzoic acid # PATENT #
Bisarylsulfonamides useful as kinase inhibitors in the treatment of inflammation and cancer # WO-2011161201-A1
Kancera_Example2-185 # 4-(3-chloro-2-methylbenzenesulfonamido)-2-hydroxybenzoic acid # PATENT #
Bisarylsulfonamides useful as kinase inhibitors in the treatment of inflammation and cancer # WO-2011161201-A1
Kancera_Example2-186 # 2-hydroxy-4-[3-methyl-5-(propan-2-yl)-1-benzothiophene-2-sulfonamido]benzoic acid #
PATENT # Bisarylsulfonamides useful as kinase inhibitors in the treatment of inflammation and cancer # WO-
2011161201-A1
Kancera_Example2-187 # 4-[4-(2,3-dihydro-1-benzofuran-5-yl)thiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-188 # 2-hydroxy-4-[3-(1-hydroxyethyl)benzenesulfonamido]benzoic acid # PATENT #
Bisarylsulfonamides useful as kinase inhibitors in the treatment of inflammation and cancer # WO-2011161201-A1
Kancera_Example2-189 # 2-hydroxy-4-(3-hydroxybenzenesulfonamido)benzoic acid # PATENT # Bisarylsulfonamides
useful as kinase inhibitors in the treatment of inflammation and cancer # WO-2011161201-A1
Kancera_Example2-190 # 2-hydroxy-4-(2-hydroxybenzenesulfonamido)benzoic acid # PATENT # Bisarylsulfonamides
useful as kinase inhibitors in the treatment of inflammation and cancer # WO-2011161201-A1
Kancera_Example2-191 # 4-[(4-chlorophenyl)methanesulfonamido]-2-hydroxybenzoic acid # PATENT #
Bisarylsulfonamides useful as kinase inhibitors in the treatment of inflammation and cancer # WO-2011161201-A1
Kancera_Example2-192 # 4-[(3-bromophenyl)methanesulfonamido]-2-hydroxybenzoic acid # PATENT #
Bisarylsulfonamides useful as kinase inhibitors in the treatment of inflammation and cancer # WO-2011161201-A1
Kancera_Example2-193 # 4-[(4-bromophenyl)methanesulfonamido]-2-hydroxybenzoic acid # PATENT #
Bisarylsulfonamides useful as kinase inhibitors in the treatment of inflammation and cancer # WO-2011161201-A1
Kancera_Example2-194 # 2-hydroxy-4-({2′-hydroxy-[1,1′-biphenyl]-4-yl}methanesulfonamido)benzoic acid # PATENT #
Bisarylsulfonamides useful as kinase inhibitors in the treatment of inflammation and cancer # WO-2011161201-A1
Kancera_Example2-195 # 4-{[4-(2,3-dihydro-1-benzofuran-5-yl)phenyl]methanesulfonamido}-2-hydroxybenzoic acid #
PATENT # Bisarylsulfonamides useful as kinase inhibitors in the treatment of inflammation and cancer # WO-
2011161201-A1
Kancera_Example2-196 # 4-({2′,5′-difluoro-[1,1′-biphenyl]-4-yl}methanesulfonamido)-2-hydroxybenzoic acid # PATENT #
Bisarylsulfonamides useful as kinase inhibitors in the treatment of inflammation and cancer # WO-2011161201-A1
Kancera_Example2-197 # 4-({[1,1′-biphenyl]-4-yl}methanesulfonamido)-2-hydroxybenzoic acid # PATENT #
Bisarylsulfonamides useful as kinase inhibitors in the treatment of inflammation and cancer # WO-2011161201-A1
Kancera_Example2-198 # 4-{[3-(2,3-dihydro-1-benzofuran-5-yl)phenyl]methanesulfonamido}-2-hydroxybenzoic acid #
PATENT # Bisarylsulfonamides useful as kinase inhibitors in the treatment of inflammation and cancer # WO-
2011161201-A1
Kancera_Example2-199 # 4-({2′,5′-difluoro-[1,1′-biphenyl]-3-yl}methanesulfonamido)-2-hydroxybenzoic acid # PATENT #
Bisarylsulfonamides useful as kinase inhibitors in the treatment of inflammation and cancer # WO-2011161201-A1
Kancera_Example2-200 # 4-(3,5-dibromothiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-201 # 4-(3,4-dibromothiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-202 # 4-(4,5-dibromothiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-203 # 4-(5-bromo-4-methylthiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-204 # 4-(5-chloro-4-methylthiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-205 # 4-(3,5-dichlorobenzenesulfonamido)-2-hydroxybenzoic acid # PATENT # Bisarylsulfonamides
useful as kinase inhibitors in the treatment of inflammation and cancer # WO-2011161201-A1
Kancera_Example2-206 # 4-[3-bromo-5-(trifluoromethyl)benzenesulfonamido]-2-hydroxybenzoic acid # PATENT #
Bisarylsulfonamides useful as kinase inhibitors in the treatment of inflammation and cancer # WO-2011161201-A1
Kancera_Example2-207 # 4-[2′,5′-difluoro-5-(trifluoromethyl)-[1,1′-biphenyl]-3-sulfonamido]-2-hydroxybenzoic acid #
PATENT # Bisarylsulfonamides useful as kinase inhibitors in the treatment of inflammation and cancer # WO-
2011161201-A1
Kancera_Example2-208 # 4-[3-(2,3-dihydro-1-benzofuran-5-yl)-5-(trifluoromethyl)benzenesulfonamido]-2-hydroxybenzoic
acid # PATENT # Bisarylsulfonamides useful as kinase inhibitors in the treatment of inflammation and cancer # WO-
2011161201-A1
Kancera_Example2-209 # 2-hydroxy-4-[2′-hydroxy-5-(trifluoromethyl)-[1,1′-biphenyl]-3-sulfonamido]benzoic acid #
PATENT # Bisarylsulfonamides useful as kinase inhibitors in the treatment of inflammation and cancer # WO-
2011161201-A1
Kancera_Example2-210 # 4-(3-chloro-2-fluorobenzenesulfonamido)-2-hydroxybenzoic acid # PATENT #
Bisarylsulfonamides useful as kinase inhibitors in the treatment of inflammation and cancer # WO-2011161201-A1
Kancera_Example2-211 # 4-(5-chloro-2-fluorobenzenesulfonamido)-2-hydroxybenzoic acid # PATENT #
Bisarylsulfonamides useful as kinase inhibitors in the treatment of inflammation and cancer # WO-2011161201-A1
Kancera_Example2-212 # 4-(2,5-dimethylfuran-3-sulfonamido)-2-hydroxybenzoic acid # PATENT # Bisarylsulfonamides
useful as kinase inhibitors in the treatment of inflammation and cancer # WO-2011161201-A1
Kancera_Example2-213 # 4-[5-(2,5-difluorophenyl)thiophene-2-sulfonamido]-2-hydroxybenzoic acid # PATENT #
Bisarylsulfonamides useful as kinase inhibitors in the treatment of inflammation and cancer # WO-2011161201-A1
Kancera_Example2-214 # 4-[5-(2,3-dihydro-1-benzofuran-5-yl)thiophene-2-sulfonamido]-2-hydroxybenzoic acid #
PATENT # Bisarylsulfonamides useful as kinase inhibitors in the treatment of inflammation and cancer # WO-
2011161201-A1
Kancera_Example2-215 # 2-hydroxy-4-(5-phenylthiophene-2-sulfonamido)benzoic acid # PATENT # Bisarylsulfonamides
useful as kinase inhibitors in the treatment of inflammation and cancer # WO-2011161201-A1
Kancera_Example2-216 # 2-hydroxy-4-[5-(2-hydroxyphenyl)thiophene-2-sulfonamido]benzoic acid # PATENT #
Bisarylsulfonamides useful as kinase inhibitors in the treatment of inflammation and cancer # WO-2011161201-A1
Kancera_Example2-217 # 2-hydroxy-4-(4-phenoxybenzenesulfonamido)benzoic acid # PATENT # Bisarylsulfonamides
useful as kinase inhibitors in the treatment of inflammation and cancer # WO-2011161201-A1
Kancera_Example2-218 # 4-(2,5-dimethylthiophene-3-sulfonamido)-2-hydroxybenzoic acid # PATENT #
Bisarylsulfonamides useful as kinase inhibitors in the treatment of inflammation and cancer # WO-2011161201-A1
Kancera_Example2-219 # 4-(4-chlorobenzenesulfonamido)-2-hydroxybenzoic acid # PATENT # Bisarylsulfonamides
useful as kinase inhibitors in the treatment of inflammation and cancer # WO-2011161201-A1
Kancera_Example2-220 # 4-benzenesulfonamido-2-hydroxybenzoic acid # PATENT # Bisarylsulfonamides useful as
kinase inhibitors in the treatment of inflammation and cancer # WO-2011161201-A1
Kancera_Example2-221 # 2-hydroxy-4-(3-nitrobenzenesulfonamido)benzoic acid # PATENT # Bisarylsulfonamides
useful as kinase inhibitors in the treatment of inflammation and cancer # WO-2011161201-A1
Kancera_Example2-223 # 2-hydroxy-4-(naphthalene-2-sulfonamido)benzoic acid # PATENT # Bisarylsulfonamides
useful as kinase inhibitors in the treatment of inflammation and cancer # WO-2011161201-A1
Kancera_Example2-224 # 4-(3-carboxybenzenesulfonamido)-2-hydroxybenzoic acid # PATENT # Bisarylsulfonamides
useful as kinase inhibitors in the treatment of inflammation and cancer # WO-2011161201-A1
Kancera_Example2-225 # 4-(4-carboxybenzenesulfonamido)-2-hydroxybenzoic acid # PATENT # Bisarylsulfonamides
useful as kinase inhibitors in the treatment of inflammation and cancer # WO-2011161201-A1
Kancera_Example2-226 # 4-(2,5-dichlorobenzenesulfonamido)-2-hydroxybenzoic acid # PATENT # Bisarylsulfonamides
useful as kinase inhibitors in the treatment of inflammation and cancer # WO-2011161201-A1
Kancera_Example2-227 # 4-(3-chlorobenzenesulfonamido)-2-hydroxybenzoic acid # PATENT # Bisarylsulfonamides
useful as kinase inhibitors in the treatment of inflammation and cancer # WO-2011161201-A1
Kancera_Example2-228 # 4-(3-bromo-5-chlorothiophene-2-sulfonamido)-2-hydroxybenzoic acid # PATENT #
Bisarylsulfonamides useful as kinase inhibitors in the treatment of inflammation and cancer # WO-2011161201-A1
Kancera_Example2-229 # 4-(4-bromothiophene-3-sulfonamido)-2-hydroxybenzoic acid # PATENT # Bisarylsulfonamides
useful as kinase inhibitors in the treatment of inflammation and cancer # WO-2011161201-A1
NSQP00513 # N-(4-{[3-cyano-1-(2-methylpropyl)-1H-indol-5-yl]oxy}phenyl)pyrrolidine-2-carboxamide # PUBLICATION #
Structure-Based Design of Potent and Selective Inhibitors of the Metabolic Kinase PFKFB3. #
10.1021/acs.jmedchem.5b00352
Merck_Example30 # N-[(S)-(1-methyl-1H-1,2,3-triazol-5-yl)(1-methyl-1H-pyrazol-3-yl)methyl]-8-(1-methyl-1H-indol-6-
yl)quinoxalin-6-amine # PATENT # Substituted quinoxaline derivatives as inhibitors of pfkfb # WO2018087021A1
Merck_Example29 # N-[(R)-(1-methyl-1H-1,2,3-triazol-5-yl)(1-methyl-1H-pyrazol-3-yl)methyl]-8-(1-methyl-1H-indol-6-
yl)quinoxalin-6-amine # PATENT # Substituted quinoxaline derivatives as inhibitors of pfkfb # WO2018087021A1
Merck_Example27 # N-[(R)-(1-methyl-1H-pyrazol-4-yl)(pyridin-3-yl)methyl]-8-{1-methyl-1H-pyrrolo[3,2-b]pyridin-6-
yl}quinoxalin-6-amine # PATENT # Substituted quinoxaline derivatives as inhibitors of pfkfb # WO2018087021A1
Merck_Example26 # N-[(S)-(1-methyl-1H-pyrazol-4-yl)(pyridin-3-yl)methyl]-8-{1-methyl-1H-pyrrolo[3,2-b]pyridin-6-
yl}quinoxalin-6-amine # PATENT # Substituted quinoxaline derivatives as inhibitors of pfkfb # WO2018087021A1
Merck_Example24 # 8-(3-methyl-1-benzofuran-5-yl)-N-[(R)-(1-methyl-1H-1,2,3-triazol-5-yl)(1-methyl-1H-pyrazol-4-
yl)methyl]quinoxalin-6-amine # PATENT # Substituted quinoxaline derivatives as inhibitors of pfkfb # WO2018087021A1
Merck_Example23 # 8-(3-methyl-1-benzofuran-5-yl)-N-[(S)-(1-methyl-1H-1,2,3-triazol-5-yl)(1-methyl-1H-pyrazol-4-
yl)methyl]quinoxalin-6-amine # PATENT # Substituted quinoxaline derivatives as inhibitors of pfkfb # WO2018087021A1
Merck_Example21 # N-[(1S)-2-methyl-1-(pyridin-3-yl)propyl]-8-(1-methyl-1H-indol-6-yl)quinoxalin-6-amine # PATENT #
Substituted quinoxaline derivatives as inhibitors of pfkfb # WO2018087021A1
Merck_Example20 # N-[(1R)-2-methyl-1-(pyridin-3-yl)propyl]-8-(1-methyl-1H-indol-6-yl)quinoxalin-6-amine # PATENT #
Substituted quinoxaline derivatives as inhibitors of pfkfb # WO2018087021A1
Merck_Example18 # 8-(1-methyl-1H-indol-6-yl)-N-[(R)-(1-methyl-1H-pyrazol-4-yl)(pyridin-3-yl)methyl]quinoxalin-6-amine
# PATENT # Substituted quinoxaline derivatives as inhibitors of pfkfb # WO2018087021A1
Merck_Example17 # 8-(1-methyl-1H-indol-6-yl)-N-[(S)-(1-methyl-1H-pyrazol-4-yl)(pyridin-3-yl)methyl]quinoxalin-6-amine
# PATENT # Substituted quinoxaline derivatives as inhibitors of pfkfb # WO2018087021A1
Merck_Example15 # N-[(R)-(1-methyl-1H-imidazol-2-yl)(1-methyl-1H-pyrazol-4-yl)methyl]-8-(1-methyl-1H-indol-6-
yl)quinoxalin-6-amine # PATENT # Substituted quinoxaline derivatives as inhibitors of pfkfb # WO2018087021A1
Merck_Example14 # N-[(S)-(1-methyl-1H-imidazol-2-yl)(1-methyl-1H-pyrazol-4-yl)methyl]-8-(1-methyl-1H-indol-6-
yl)quinoxalin-6-amine # PATENT # Substituted quinoxaline derivatives as inhibitors of pfkfb # WO2018087021A1
Merck_Example12 # N-[(R)-(1-methyl-1H-imidazol-5-yl)(1-methyl-1H-pyrazol-4-yl)methyl]-8-(1-methyl-1H-indol-6-
yl)quinoxalin-6-amine # PATENT # Substituted quinoxaline derivatives as inhibitors of pfkfb # WO2018087021A1
Merck_Example11 # N-[(S)-(1-methyl-1H-imidazol-5-yl)(1-methyl-1H-pyrazol-4-yl)methyl]-8-(1-methyl-1H-indol-6-
yl)quinoxalin-6-amine # PATENT # Substituted quinoxaline derivatives as inhibitors of pfkfb # WO2018087021A1
Merck_Example9 # N-[(R)-(1-methyl-1H-1,2,3-triazol-5-yl)(1-methyl-1H-pyrazol-4-yl)methyl]-8-(1-methyl-1H-indol-6-
yl)quinoxalin-6-amine # PATENT # Substituted quinoxaline derivatives as inhibitors of pfkfb # WO2018087021A1
Merck_Example8 # N-[(S)-(1-methyl-1H-1,2,3-triazol-5-yl)(1-methyl-1H-pyrazol-4-yl)methyl]-8-(1-methyl-1H-indol-6-
yl)quinoxalin-6-amine # PATENT # Substituted quinoxaline derivatives as inhibitors of pfkfb # WO2018087021A1
Merck_Example28 # N-[(1-methyl-1H-1,2,3-triazol-5-yl)(1-methyl-1H-pyrazol-3-yl)methyl]-8-(1-methyl-1H-indol-6-
yl)quinoxalin-6-amine # PATENT # Substituted quinoxaline derivatives as inhibitors of pfkfb # WO2018087021A1
Merck_Example25 # N-[(1-methyl-1H-pyrazol-4-yl)(pyridin-3-yl)methyl]-8-{1-methyl-1H-pyrrolo[3,2-b]pyridin-6-
yl}quinoxalin-6-amine # PATENT # Substituted quinoxaline derivatives as inhibitors of pfkfb # WO2018087021A1
Merck_Example22 # 8-(3-methyl-1-benzofuran-5-yl)-N-[(1-methyl-1H-1,2,3-triazol-5-yl)(1-methyl-1H-pyrazol-4-
yl)methyl]quinoxalin-6-amine # PATENT # Substituted quinoxaline derivatives as inhibitors of pfkfb # WO2018087021A1
Merck_Example19 # N-[2-methyl-1-(pyridin-3-yl)propyl]-8-(1-methyl-1H-indol-6-yl)quinoxalin-6-amine # PATENT #
Substituted quinoxaline derivatives as inhibitors of pfkfb # WO2018087021A1
Merck_Example16 # 8-(1-methyl-1H-indol-6-yl)-N-[(1-methyl-1H-pyrazol-4-yl)(pyridin-3-yl)methyl]quinoxalin-6-amine #
PATENT # Substituted quinoxaline derivatives as inhibitors of pfkfb # WO2018087021A1
Merck_Example13 # N-[(1-methyl-1H-imidazol-2-yl)(1-methyl-1H-pyrazol-4-yl)methyl]-8-(1-methyl-1H-indol-6-
yl)quinoxalin-6-amine # PATENT # Substituted quinoxaline derivatives as inhibitors of pfkfb # WO2018087021A1
Merck_Example10 # N-[(1-methyl-1H-imidazol-5-yl)(1-methyl-1H-pyrazol-4-yl)methyl]-8-(1-methyl-1H-indol-6-
yl)quinoxalin-6-amine # PATENT # Substituted quinoxaline derivatives as inhibitors of pfkfb # WO2018087021A1
Merck_Example7 # N-[(1-methyl-1H-1,2,3-triazol-5-yl)(1-methyl-1H-pyrazol-4-yl)methyl]-8-(1-methyl-1H-indol-6-
yl)quinoxalin-6-amine # PATENT # Substituted quinoxaline derivatives as inhibitors of pfkfb # WO2018087021A1
Merck_Example6 # N-[(R)-(6-methoxypyridazin-3-yl)(pyridin-3-yl)methyl]-8-(1-methyl-1H-indol-6-yl)quinoxalin-6-amine #
PATENT # Substituted quinoxaline derivatives as inhibitors of pfkfb # WO2018087021A1
Merck_Example5 # N-[(S)-(6-methoxypyridazin-3-yl)(pyridin-3-yl)methyl]-8-(1-methyl-1H-indol-6-yl)quinoxalin-6-amine #
PATENT # Substituted quinoxaline derivatives as inhibitors of pfkfb # WO2018087021A1
Merck_Example4 # N-[(6-methoxypyridazin-3-yl)(pyridin-3-yl)methyl]-8-(1-methyl-1H-indol-6-yl)quinoxalin-6-amine #
PATENT # Substituted quinoxaline derivatives as inhibitors of pfkfb # WO2018087021A1
Merck_Example3 # 6-[(R)-{[8-(1-methyl-1H-indol-6-yl)quinoxalin-6-yl]amino}(pyridin-3-yl)methyl]-2,3-dihydropyridazin-3-
one # PATENT # Substituted quinoxaline derivatives as inhibitors of pfkfb # WO2018087021A1
Merck_Example2 # 6-[(S)-{[8-(1-methyl-1H-indol-6-yl)quinoxalin-6-yl]amino}(pyridin-3-yl)methyl]-2,3-dihydropyridazin-3-
one # PATENT # Substituted quinoxaline derivatives as inhibitors of pfkfb # WO2018087021A1
Merck_Example1 # 6-({[8-(1-methyl-1H-indol-6-yl)quinoxalin-6-yl]amino}(pyridin-3-yl)methyl)-2,3-dihydropyridazin-3-one
# PATENT # Substituted quinoxaline derivatives as inhibitors of pfkfb # WO2018087021A1
Selvita_Example_215 # 3-{[8-(4-bromophenyl)quinoxalin-6-yl]amino}-N-(1-methylpyrrolidin-3-yl)pyridine-4-carboxamide #
PATENT # Substituted quinoxaline derivatives # WO2016180537A1
Selvita_Example_214 # 3-{[8-(2-amino-1,3-benzothiazol-5-yl)quinoxalin-6-yl]amino}-N-(1-methylpyrrolidin-3-yl)pyridine-4-
carboxamide # PATENT # Substituted quinoxaline derivatives # WO2016180537A1
Selvita_Example_213 # N-(4-methanesulfonylpyridin-3-yl)-8-[4-(pentafluoro- [symbol] -sulfanyl)phenyl]quinoxalin-6-amine #
PATENT # Substituted quinoxaline derivatives # WO2016180537A1
Selvita_Example_212 # 8-(4-bromo-2-methylphenyl)-N-(4-methanesulfonylpyridin-3-yl)quinoxalin-6-amine # PATENT #
Substituted quinoxaline derivatives # WO2016180537A1
Selvita_Example_211 # 8-(4-bromo-3-fluorophenyl)-N-(4-methanesulfonylpyridin-3-yl)quinoxalin-6-amine # PATENT #
Substituted quinoxaline derivatives # WO2016180537A1
Selvita_Example_210 # N-(4-methanesulfonylpyridin-3-yl)-8-[3-(methylsulfanyl)phenyl]quinoxalin-6-amine # PATENT #
Substituted quinoxaline derivatives # WO2016180537A1
Selvita_Example_209 # 2-{[8-(1-methyl-1H-indol-6-yl)quinoxalin-6-yl]amino}-N-(oxan-3-yl)benzene-1-sulfonamide #
PATENT # Substituted quinoxaline derivatives # WO2016180537A1
Selvita_Example_208 # 2-{[8-(1-methyl-1H-indol-6-yl)quinoxalin-6-yl]amino}-N-(1-methylpiperidin-3-yl)benzene-1-
sulfonamide # PATENT # Substituted quinoxaline derivatives # WO2016180537A1
Selvita_Example_207 # 3-{[8-(1-methyl-1H-indol-6-yl)quinoxalin-6-yl]amino}-N-(pyridin-3-yl)pyridine-4-carboxamide #
PATENT # Substituted quinoxaline derivatives # WO2016180537A1
Selvita_Example_206 # N-(1-acetylazetidin-3-yl)-3-{[8-(3-methyl-1-benzothiophen-5-yl)quinoxalin-6-yl]amino}pyridine-4-
carboxamide # PATENT # Substituted quinoxaline derivatives # WO2016180537A1
Selvita_Example_205 # 3-{[8-(3-methyl-1-benzothiophen-5-yl)quinoxalin-6-yl]amino}pyridine-4-carboxamide # PATENT #
Substituted quinoxaline derivatives # WO2016180537A1
Selvita_Example_204 # N-(5-bromopyrimidin-4-yl)-8-(1-methyl-1H-indol-6-yl)quinoxalin-6-amine # PATENT # Substituted
quinoxaline derivatives # WO2016180537A1
Selvita_Example_203 # 8-(2-amino-1-benzothiophen-5-yl)-N-(4-methanesulfonylpyridin-3-yl)quinoxalin-6-amine #
PATENT # Substituted quinoxaline derivatives # WO2016180537A1
Selvita_Example_202 # N-(4-methanesulfonylpyridin-3-yl)-8-[2-(methylamino)-1,3-benzothiazol-5-yl]quinoxalin-6-amine #
PATENT # Substituted quinoxaline derivatives # WO2016180537A1
Selvita_Example_201 # 8-[2-(dimethylamino)-1,3-benzothiazol-5-yl]-N-(4-methanesulfonylpyridin-3-yl)quinoxalin-6-amine
# PATENT # Substituted quinoxaline derivatives # WO2016180537A1
Selvita_Example_200 # N-(5-{7-[(4-methanesulfonylpyridin-3-yl)amino]quinoxalin-5-yl}-1-benzothiophen-2-yl)acetamide #
PATENT # Substituted quinoxaline derivatives # WO2016180537A1
Selvita_Example_199 # 2-{[8-(3-methyl-1-benzothiophen-5-yl)quinoxalin-6-yl]amino}-N-(1-methylpyrrolidin-3-yl)benzene-
1-sulfonamide # PATENT # Substituted quinoxaline derivatives # WO2016180537A1
Selvita_Example_198 # 3-{[8-(1-methyl-1H-indol-6-yl)quinoxalin-6-yl]oxy}pyridine-4-carboxylic acid # PATENT #
Substituted quinoxaline derivatives # WO2016180537A1
Selvita_Example_197 # 3-{[8-(4-fluoro-1-methyl-1H-indol-6-yl)quinoxalin-6-yl]amino}-N-(1-methylpyrrolidin-3-yl)pyridine-
4-carboxamide # PATENT # Substituted quinoxaline derivatives # WO2016180537A1
Selvita_Example_195 # 8-(1,5-dimethyl-1H-indol-6-yl)-N-(4-methanesulfonylpyridin-3-yl)quinoxalin-6-amine # PATENT #
Substituted quinoxaline derivatives # WO2016180537A1
Selvita_Example_194 # 3-{[8-(3-methyl-1-benzothiophen-5-yl)quinoxalin-6-yl]amino}-N-[(3R)-1-methylpyrrolidin-3-
yl]pyridine-4-carboxamide # PATENT # Substituted quinoxaline derivatives # WO2016180537A1
Selvita_Example_193 # 3-{[8-(3-methyl-1-benzothiophen-5-yl)quinoxalin-6-yl]amino}-N-[(3S)-1-methylpyrrolidin-3-
yl]pyridine-4-carboxamide # PATENT # Substituted quinoxaline derivatives # WO2016180537A1
Selvita_Example_192 # 8-(3,5-diethylphenyl)-N-(4-methanesulfonylpyridin-3-yl)quinoxalin-6-amine # PATENT #
Substituted quinoxaline derivatives # WO2016180537A1
Selvita_Example_191 # N-(2-methanesulfonylphenyl)-8-(3-methyl-1-benzothiophen-5-yl)quinoxalin-6-amine # PATENT #
Substituted quinoxaline derivatives # WO2016180537A1
Selvita_Example_190 # 8-(2-amino-1,3-benzothiazol-5-yl)-N-(2-methanesulfonylphenyl)quinoxalin-6-amine # PATENT #
Substituted quinoxaline derivatives # WO2016180537A1
Selvita_Example_189 # 8-(1,4-dimethyl-1H-indol-6-yl)-N-(4-methanesulfonylpyridin-3-yl)quinoxalin-6-amine # PATENT #
Substituted quinoxaline derivatives # WO2016180537A1
Selvita_Example_188 # 8-(4-fluorophenyl)-N-(4-methanesulfonylpyridin-3-yl)quinoxalin-6-amine # PATENT # Substituted
quinoxaline derivatives # WO2016180537A1
Selvita_Example_187 # N-methyl-2-{[8-(1-methyl-1H-indol-6-yl)quinoxalin-6-yl]amino}-N-[(pyrimidin-5-yl)methyl]benzene-
1-sulfonamide # PATENT # Substituted quinoxaline derivatives # WO2016180537A1
Selvita_Example_186 # 8-(4-amino-3-fluorophenyl)-N-(4-methanesulfonylpyridin-3-yl)quinoxalin-6-amine # PATENT #
Substituted quinoxaline derivatives # WO2016180537A1
Selvita_Example_185 # N-(4-methanesulfonylpyridin-3-yl)-8-[4-(trifluoromethyl)phenyl]quinoxalin-6-amine # PATENT #
Substituted quinoxaline derivatives # WO2016180537A1
Selvita_Example_184 # N-(4-methanesulfonylpyridin-3-yl)-8-(1-propyl-1H-indol-6-yl)quinoxalin-6-amine # PATENT #
Substituted quinoxaline derivatives # WO2016180537A1
Selvita_Example_183 # 2-{[8-(1-methyl-1H-indol-6-yl)quinoxalin-6-yl]amino}-N-(1-methylpyrrolidin-3-yl)benzamide #
PATENT # Substituted quinoxaline derivatives # WO2016180537A1
Selvita_Example_182 # N-methyl-3-{[8-(1-methyl-1H-indol-6-yl)quinoxalin-6-yl]amino}-N-(1-methylpyrrolidin-3-yl)pyridine-
4-carboxamide # PATENT # Substituted quinoxaline derivatives # WO2016180537A1
Selvita_Example_181 # 3-{[8-(1-methyl-1H-indol-6-yl)quinoxalin-6-yl]amino}-N-(1-methylazetidin-3-yl)pyridine-4-
carboxamide # PATENT # Substituted quinoxaline derivatives # WO2016180537A1
Selvita_Example_179 # 3-{[8-(1-methyl-1H-indol-6-yl)quinoxalin-6-yl]amino}pyridine-4-carboxylic acid # PATENT #
Substituted quinoxaline derivatives # WO2016180537A1
Selvita_Example_178 # 2-{[8-(1-methyl-1H-indol-6-yl)quinoxalin-6-yl]amino}benzoic acid # PATENT # Substituted
quinoxaline derivatives # WO2016180537A1
Selvita_Example_176 # N-{2-[(dimethylamino)methyl]phenyl}-8-(1-methyl-1H-indol-6-yl)quinoxalin-6-amine # PATENT #
Substituted quinoxaline derivatives # WO2016180537A1
Selvita_Example_175 # N-{4-[(dimethylamino)methyl]pyridin-3-yl}-8-(1-methyl-1H-indol-6-yl)quinoxalin-6-amine #
PATENT # Substituted quinoxaline derivatives # WO2016180537A1
Selvita_Example_174 # 8-(2-amino-1,3-benzothiazol-6-yl)-N-(4-methanesulfonylpyridin-3-yl)quinoxalin-6-amine #
PATENT # Substituted quinoxaline derivatives # WO2016180537A1
Selvita_Example_173 # 2-{[8-(1-methyl-1H-indol-6-yl)quinoxalin-6-yl]amino}-N-[(1-methyl-1H-pyrazol-4-
yl)methyl]benzene-1-sulfonamide # PATENT # Substituted quinoxaline derivatives # WO2016180537A1
Selvita_Example_172 # 2-{[8-(1-methyl-1H-indol-6-yl)quinoxalin-6-yl]amino}-N-[(oxan-4-yl)methyl]benzene-1-sulfonamide
# PATENT # Substituted quinoxaline derivatives # WO2016180537A1
Selvita_Example_171 # 2-{[8-(1-methyl-1H-indol-6-yl)quinoxalin-6-yl]amino}-N-(1-methylpyrrolidin-3-yl)benzene-1-
sulfonamide # PATENT # Substituted quinoxaline derivatives # WO2016180537A1
Selvita_Example_170 # N-(4-methanesulfonylpyridin-3-yl)-8-(1-methyl-1H-1,2,3-benzotriazol-6-yl)quinoxalin-6-amine #
PATENT # Substituted quinoxaline derivatives # WO2016180537A1
Selvita_Example_169 # N-(4-methanesulfonylpyridin-3-yl)-8-(2-methyl-1,3-benzothiazol-5-yl)quinoxalin-6-amine #
PATENT # Substituted quinoxaline derivatives # WO2016180537A1
Selvita_Example_168 # 2-{[8-(1-methyl-1H-indol-6-yl)quinoxalin-6-yl]amino}-N-[(1-methylpyrrolidin-3-yl)methyl]benzene-
1-sulfonamide # PATENT # Substituted quinoxaline derivatives # WO2016180537A1
Selvita_Example_167 # N-(4-methanesulfonylpyridin-3-yl)-8-(1-methyl-1H-1,2,3-benzotriazol-5-yl)quinoxalin-6-amine #
PATENT # Substituted quinoxaline derivatives # WO2016180537A1
Selvita_Example_166 # 8-(1-ethyl-1H-indol-6-yl)-N-(4-methanesulfonylpyridin-3-yl)quinoxalin-6-amine # PATENT #
Substituted quinoxaline derivatives # WO2016180537A1
Selvita_Example_165 # N-(3-{7-[(4-methanesulfonylpyridin-3-yl)amino]quinoxalin-5-yl}phenyl)pyrrolidine-2-carboxamide
# PATENT # Substituted quinoxaline derivatives # WO2016180537A1
Selvita_Example_164 # N-(4-{7-[(4-methanesulfonylpyridin-3-yl)amino]quinoxalin-5-yl}phenyl)pyrrolidine-2-carboxamide
# PATENT # Substituted quinoxaline derivatives # WO2016180537A1
Selvita_Example_163 # N-(4-methanesulfonylpyridin-3-yl)-8-[3-(trifluoromethoxy)phenyl]quinoxalin-6-amine # PATENT #
Substituted quinoxaline derivatives # WO2016180537A1
Selvita_Example_162 # 8-(2-amino-1,3-benzothiazol-5-yl)-N-(4-methanesulfonylpyridin-3-yl)quinoxalin-6-amine #
PATENT # Substituted quinoxaline derivatives # WO2016180537A1
Selvita_Example_161 # 8-(1,2-benzothiazol-5-yl)-N-(4-methanesulfonylpyridin-3-yl)quinoxalin-6-amine # PATENT #
Substituted quinoxaline derivatives # WO2016180537A1
Selvita_Example_160 # 2-(2-aminopyrimidin-4-yl)-3-{[8-(1-methyl-1H-indol-6-yl)quinoxalin-6-yl]amino}pyridine-4-
carboxylic acid # PATENT # Substituted quinoxaline derivatives # WO2016180537A1
Selvita_Example_158 # 8-(3-bromophenyl)-N-(4-methanesulfonylpyridin-3-yl)quinoxalin-6-amine # PATENT #
Substituted quinoxaline derivatives # WO2016180537A1
Selvita_Example_157 # 8-(4-bromophenyl)-N-(4-methanesulfonylpyridin-3-yl)quinoxalin-6-amine # PATENT #
Substituted quinoxaline derivatives # WO2016180537A1
Selvita_Example_154 # 8-[1-(difluoromethyl)-1H-indol-6-yl]-N-(4-methanesulfonylpyridin-3-yl)quinoxalin-6-amine #
PATENT # Substituted quinoxaline derivatives # WO2016180537A1
Selvita_Example_153 # N-(4-methanesulfonylpyridin-3-yl)-8-(5-methoxy-2-methylphenyl)quinoxalin-6-amine # PATENT #
Substituted quinoxaline derivatives # WO2016180537A1
Selvita_Example_152 # N-(4-methanesulfonylpyridin-3-yl)-8-(4-methoxyphenyl)quinoxalin-6-amine # PATENT #
Substituted quinoxaline derivatives # WO2016180537A1
Selvita_Example_151 # 3-{[8-(3-methyl-1-benzothiophen-5-yl)quinoxalin-6-yl]amino}-N-(1-methylpyrrolidin-3-yl)pyridine-
4-carboxamide # PATENT # Substituted quinoxaline derivatives # WO2016180537A1
Selvita_Example_150 # 3-{[8-(3-methyl-1-benzothiophen-5-yl)quinoxalin-6-yl]amino}pyridine-4-carbonitrile # PATENT #
Substituted quinoxaline derivatives # WO2016180537A1
Selvita_Example_149 # 5-{[8-(1-methyl-1H-indol-6-yl)quinoxalin-6-yl]amino}pyrimidine-4-carboxamide # PATENT #
Substituted quinoxaline derivatives # WO2016180537A1
Selvita_Example_148 # N-(1-acetylpiperidin-3-yl)-3-{[8-(1-methyl-1H-indol-6-yl)quinoxalin-6-yl]amino}pyridine-4-
carboxamide # PATENT # Substituted quinoxaline derivatives # WO2016180537A1
Selvita_Example_147 # N-(1-acetylpiperidin-4-yl)-3-{[8-(1-methyl-1H-indol-6-yl)quinoxalin-6-yl]amino}pyridine-4-
carboxamide # PATENT # Substituted quinoxaline derivatives # WO2016180537A1
Selvita_Example_146 # 4-[(8-chloroquinoxalin-6-yl)amino]pyridine-3-carbonitrile # PATENT # Substituted quinoxaline
derivatives # WO2016180537A1
Selvita_Example_145 # 4-{[8-(1-methyl-1H-indol-6-yl)quinoxalin-6-yl]amino}pyridine-3-carbonitrile # PATENT #
Substituted quinoxaline derivatives # WO2016180537A1
Selvita_Example_144 # 3-{[8-(1-methyl-1H-indol-6-yl)quinoxalin-6-yl]amino}-N-[(pyridazin-3-yl)methyl]pyridine-4-
carboxamide # PATENT # Substituted quinoxaline derivatives # WO2016180537A1
Selvita_Example_143 # N-[(1-methyl-1H-imidazol-5-yl)methyl]-3-{[8-(1-methyl-1H-indol-6-yl)quinoxalin-6-
yl]amino}pyridine-4-carboxamide # PATENT # Substituted quinoxaline derivatives # WO2016180537A1
Selvita_Example_142 # 3-{[8-(1-methyl-1H-indol-6-yl)quinoxalin-6-yl]amino}-N-[(1-methylpyrrolidin-3-yl)methyl]pyridine-4-
carboxamide # PATENT # Substituted quinoxaline derivatives # WO2016180537A1
Selvita_Example_141 # N-[(4-acetylmorpholin-2-yl)methyl]-3-{[8-(1-methyl-1H-indol-6-yl)quinoxalin-6-yl]amino}pyridine-4-
carboxamide # PATENT # Substituted quinoxaline derivatives # WO2016180537A1
Selvita_Example_140 # N-[(1-acetylazetidin-3-yl)methyl]-3-{[8-(1-methyl-1H-indol-6-yl)quinoxalin-6-yl]amino}pyridine-4-
carboxamide # PATENT # Substituted quinoxaline derivatives # WO2016180537A1
Selvita_Example_137 # 3-{[8-(1-methyl-1H-indol-6-yl)quinoxalin-6-yl]amino}-N-[(4-methylmorpholin-2-yl)methyl]pyridine-
4-carboxamide # PATENT # Substituted quinoxaline derivatives # WO2016180537A1
Selvita_Example_136 # N-[(4-acetylmorpholin-3-yl)methyl]-3-{[8-(1-methyl-1H-indol-6-yl)quinoxalin-6-yl]amino}pyridine-4-
carboxamide # PATENT # Substituted quinoxaline derivatives # WO2016180537A1
Selvita_Example_135 # 3-{[8-(1-methyl-1H-indol-6-yl)quinoxalin-6-yl]amino}-N-[(morpholin-3-yl)methyl]pyridine-4-
carboxamide # PATENT # Substituted quinoxaline derivatives # WO2016180537A1
Selvita_Example_132 # 8-(3-aminophenyl)-N-(4-methanesulfonylpyridin-3-yl)quinoxalin-6-amine # PATENT # Substituted
quinoxaline derivatives # WO2016180537A1
Selvita_Example_131 # 8-(4-aminophenyl)-N-(4-methanesulfonylpyridin-3-yl)quinoxalin-6-amine # PATENT # Substituted
quinoxaline derivatives # WO2016180537A1
Selvita_Example_130 # N-(4-methanesulfonylpyridin-3-yl)-8-[3-(propan-2-yloxy)phenyl]quinoxalin-6-amine # PATENT #
Substituted quinoxaline derivatives # WO2016180537A1
Selvita_Example_129 # 8-(3-ethoxyphenyl)-N-(4-methanesulfonylpyridin-3-yl)quinoxalin-6-amine # PATENT #
Substituted quinoxaline derivatives # WO2016180537A1
Selvita_Example_128 # 2-{7-[(4-methanesulfonylpyridin-3-yl)amino]quinoxalin-5-yl}-4-methylbenzamide # PATENT #
Substituted quinoxaline derivatives # WO2016180537A1
Selvita_Example_127 # 3-{[8-(1-methyl-1H-indol-6-yl)quinoxalin-6-yl]amino}-N-(2-oxopiperidin-4-yl)pyridine-4-
carboxamide # PATENT # Substituted quinoxaline derivatives # WO2016180537A1
Selvita_Example_126 # N-cyclohexyl-3-{[8-(1-methyl-1H-indol-6-yl)quinoxalin-6-yl]amino}pyridine-4-carboxamide #
PATENT # Substituted quinoxaline derivatives # WO2016180537A1
Selvita_Example_125 # 3-{methyl[8-(1-methyl-1H-indol-6-yl)quinoxalin-6-yl]amino}-N-(pyrimidin-5-yl)pyridine-4-
carboxamide # PATENT # Substituted quinoxaline derivatives # WO2016180537A1
Selvita_Example_124 # 3-{[8-(1-methyl-1H-indol-6-yl)quinoxalin-6-yl]amino}-N-(1-methylpiperidin-3-yl)pyridine-4-
carboxamide # PATENT # Substituted quinoxaline derivatives # WO2016180537A1
Selvita_Example_123 # 3-{[8-(1-methyl-1H-indol-6-yl)quinoxalin-6-yl]amino}-N-(1-methylpiperidin-4-yl)pyridine-4-
carboxamide # PATENT # Substituted quinoxaline derivatives # WO2016180537A1
Selvita_Example_122 # 3-{[8-(1-methyl-1H-indol-6-yl)quinoxalin-6-yl]amino}-N-(1-methyl-6-oxopiperidin-3-yl)pyridine-4-
carboxamide # PATENT # Substituted quinoxaline derivatives # WO2016180537A1
Selvita_Example_121 # N-(1-acetylpyrrolidin-3-yl)-3-{[8-(1-methyl-1H-indol-6-yl)quinoxalin-6-yl]amino}pyridine-4-
carboxamide # PATENT # Substituted quinoxaline derivatives # WO2016180537A1
Selvita_Example_120 # 8-(2,1,3-benzoxadiazol-5-yl)-N-(4-methanesulfonylpyridin-3-yl)quinoxalin-6-amine # PATENT #
Substituted quinoxaline derivatives # WO2016180537A1
Selvita_Example_119 # 4-methanesulfonyl-3-{[8-(1-methyl-1H-indol-6-yl)quinoxalin-6-yl]amino}benzohydrazide #
PATENT # Substituted quinoxaline derivatives # WO2016180537A1
Selvita_Example_118 # 8-(2H-1,2,3-benzotriazol-5-yl)-N-(4-methanesulfonylpyridin-3-yl)quinoxalin-6-amine # PATENT #
Substituted quinoxaline derivatives # WO2016180537A1
Selvita_Example_117 # 8-(2,1,3-benzothiadiazol-5-yl)-N-(4-methanesulfonylpyridin-3-yl)quinoxalin-6-amine # PATENT #
Substituted quinoxaline derivatives # WO2016180537A1
Selvita_Example_116 # 4-methanesulfonyl-3-{[8-(1-methyl-1H-indol-6-yl)quinoxalin-6-yl]amino}benzamide # PATENT #
Substituted quinoxaline derivatives # WO2016180537A1
Selvita_Example_115 # methyl 4-methanesulfonyl-3-{[8-(1-methyl-1H-indol-6-yl)quinoxalin-6-yl]amino}benzoate #
PATENT # Substituted quinoxaline derivatives # WO2016180537A1
Selvita_Example_113 # N-(1-methyl-1H-1,2,3-triazol-5-yl)-8-(1-methyl-1H-indol-6-yl)quinoxalin-6-amine # PATENT #
Substituted quinoxaline derivatives # WO2016180537A1
Selvita_Example_110 # N-(4-methanesulfonylpyridin-3-yl)-8-(3-methyl-1-benzothiophen-5-yl)quinoxalin-6-amine #
PATENT # Substituted quinoxaline derivatives # WO2016180537A1
Selvita_Example_104 # N-(4-methanesulfonylpyridin-3-yl)-N-methyl-8-(1-methyl-1H-indol-6-yl)quinoxalin-6-amine #
PATENT # Substituted quinoxaline derivatives # WO2016180537A1
Selvita_Example_103 # N-(2-methanesulfonyl-5-nitrophenyl)-8-(3-methyl-1-benzofuran-5-yl)quinoxalin-6-amine #
PATENT # Substituted quinoxaline derivatives # WO2016180537A1
Selvita_Example_102 # 6-methanesulfonyl-N1-[8-(3-methyl-1-benzofuran-5-yl)quinoxalin-6-yl]benzene-1,3-diamine #
PATENT # Substituted quinoxaline derivatives # WO2016180537A1
Selvita_Example_101 # 3-{[8-(1-methyl-1H-indol-6-yl)quinoxalin-6-yl]amino}-N-(oxan-4-yl)pyridine-4-carboxamide #
PATENT # Substituted quinoxaline derivatives # WO2016180537A1
Selvita_Example_100 # 2-{[8-(1-methyl-1H-indol-6-yl)quinoxalin-6-yl]amino}-N-(pyrimidin-5-yl)benzene-1-sulfonamide #
PATENT # Substituted quinoxaline derivatives # WO2016180537A1
Selvita_Example_99 # 3-{[8-(1-methyl-1H-indol-6-yl)quinoxalin-6-yl]amino}-N-(1-methylpyrrolidin-3-yl)pyridine-4-
carboxamide # PATENT # Substituted quinoxaline derivatives # WO2016180537A1
Selvita_Example_98 # N-(1-acetylazetidin-3-yl)-3-{[8-(1-methyl-1H-indol-6-yl)quinoxalin-6-yl]amino}pyridine-4-
carboxamide # PATENT # Substituted quinoxaline derivatives # WO2016180537A1
Selvita_Example_97 # 3-{[8-(1-methyl-1H-indol-6-yl)quinoxalin-6-yl]amino}-N-(1-methyl-2-oxopiperidin-4-yl)pyridine-4-
carboxamide # PATENT # Substituted quinoxaline derivatives # WO2016180537A1
Selvita_Example_96 # 3-{[8-(1-methyl-1H-indol-6-yl)quinoxalin-6-yl]amino}-N-phenylpyridine-4-carboxamide # PATENT #
Substituted quinoxaline derivatives # WO2016180537A1
Selvita_Example_95 # 3-{methyl[8-(1-methyl-1H-indol-6-yl)quinoxalin-6-yl]amino}pyridine-4-carboxamide # PATENT #
Substituted quinoxaline derivatives # WO2016180537A1
Selvita_Example_94 # N-[2-methanesulfonyl-5-(1,3-oxazol-2-yl)phenyl]-8-(1-methyl-1H-indol-6-yl)quinoxalin-6-amine #
PATENT # Substituted quinoxaline derivatives # WO2016180537A1
Selvita_Example_93 # N-[2-methanesulfonyl-5-(1-methyl-1H-pyrazol-5-yl)phenyl]-8-(1-methyl-1H-indol-6-yl)quinoxalin-6-
amine # PATENT # Substituted quinoxaline derivatives # WO2016180537A1
Selvita_Example_92 # 3-{[8-(1-methyl-1H-indol-6-yl)quinoxalin-6-yl]amino}-N-(1-methyl-1H-pyrazol-4-yl)pyridine-4-
carboxamide # PATENT # Substituted quinoxaline derivatives # WO2016180537A1
Selvita_Example_91 # 3-{methyl[8-(1-methyl-1H-indol-yl)quinoxalin-6-yl]amino}pyridine-4-carbonitrile # PATENT #
Substituted quinoxaline derivatives # WO2016180537A1
Selvita_Example_90 # 4-cyano-3-{[8-(1-methyl-1H-indol-6-yl)quinoxalin-6-yl]amino}pyridin-1-ium-1-olate # PATENT #
Substituted quinoxaline derivatives # WO2016180537A1
Selvita_Example_89 # 2-{[8-(1-methyl-1H-indol-6-yl)quinoxalin-6-yl]amino}benzamide # PATENT # Substituted
quinoxaline derivatives # WO2016180537A1
Selvita_Example_88 # 2-{[8-(1-methyl-1H-indol-6-yl)quinoxalin-6-yl]amino}benzonitrile # PATENT # Substituted
quinoxaline derivatives # WO2016180537A1
Selvita_Example_85 # 2-{[8-(1-methyl-1H-indol-6-yl)quinoxalin-6-yl]amino}-N-[(pyrimidin-5-yl)methyl]benzene-1-
sulfonamide # PATENT # Substituted quinoxaline derivatives # WO2016180537A1
Selvita_Example_84 # 8-(1-methyl-1H-indol-6-yl)-N-{2H,3H,4H-pyrido[4,3-b][1,4]oxazin-8-yl}quinoxalin-6-amine #
PATENT # Substituted quinoxaline derivatives # WO2016180537A1
Selvita_Example_83 # N-[4-(1-methyl-1H-imidazol-4-yl)pyridin-3-yl]-8-(1-methyl-1H-indol-6-yl)quinoxalin-6-amine #
PATENT # Substituted quinoxaline derivatives # WO2016180537A1
Selvita_Example_82 # 1-[4-(3-{[8-(1-methyl-1H-indol-6-yl)quinoxalin-6-yl]amino}pyridin-4-yl)piperazin-1-yl]ethan-1-one #
PATENT # Substituted quinoxaline derivatives # WO2016180537A1
Selvita_Example_81 # N-(3-methanesulfonylpyridin-2-yl)-8-(1-methyl-1H-indol-6-yl)quinoxalin-6-amine # PATENT #
Substituted quinoxaline derivatives # WO2016180537A1
Selvita_Example_76 # 8-[2-(dimethylamino)-5-methylphenyl]-N-(4-methanesulfonylpyridin-3-yl)quinoxalin-6-amine #
PATENT # Substituted quinoxaline derivatives # WO2016180537A1
Selvita_Example_75 # 8-(3-amino-4-methylphenyl)-N-(4-methanesulfonylpyridin-3-yl)quinoxalin-6-amine # PATENT #
Substituted quinoxaline derivatives # WO2016180537A1
Selvita_Example_74 # N-(4-methanesulfonylpyridin-3-yl)-8-(2-methoxy-5-methylphenyl)quinoxalin-6-amine # PATENT #
Substituted quinoxaline derivatives # WO2016180537A1
Selvita_Example_73 # 8-(5-fluoro-1-methyl-1H-indol-6-yl)-N-(4-methanesulfonylpyridin-3-yl)quinoxalin-6-amine #
PATENT # Substituted quinoxaline derivatives # WO2016180537A1
Selvita_Example_72 # 4-methanesulfonyl-3-{[8-(3-methyl-1-benzofuran-5-yl)quinoxalin-6-yl]amino}pyridin-1-ium-1-olate #
PATENT # Substituted quinoxaline derivatives # WO2016180537A1
Selvita_Example_71 # 8-(4-fluoro-1-methyl-1H-indol-6-yl)-N-(4-methanesulfonylpyridin-3-yl)quinoxalin-6-amine #
PATENT # Substituted quinoxaline derivatives # WO2016180537A1
Selvita_Example_69 # N-(4-chloropyridin-3-yl)-8-(1-methyl-1H-indol-6-yl)quinoxalin-6-amine # PATENT # Substituted
quinoxaline derivatives # WO2016180537A1
Selvita_Example_68 # 8-(1-methyl-1H-indol-6-yl)-N-[4-(2H-1,2,3,4-tetrazol-5-yl)pyridin-3-yl]quinoxalin-6-amine #
PATENT # Substituted quinoxaline derivatives # WO2016180537A1
Selvita_Example_67 # 2-{7-[(4-methanesulfonylpyridin-3-yl)amino]quinoxalin-5-yl}-4-methylphenol # PATENT #
Substituted quinoxaline derivatives # WO2016180537A1
Selvita_Example_66 # 8-(2-amino-5-methylphenyl)-N-(4-methanesulfonylpyridin-3-yl)quinoxalin-6-amine # PATENT #
Substituted quinoxaline derivatives # WO2016180537A1
Selvita_Example_65 # 8-(3-ethylphenyl)-N-(4-methanesulfonylpyridin-3-yl)quinoxalin-6-amine # PATENT # Substituted
quinoxaline derivatives # WO2016180537A1
Selvita_Example_63 # 8-(3,3-dimethyl-2,3-dihydro-1-benzofuran-5-yl)-N-(4-methanesulfonylpyridin-3-yl)quinoxalin-6-
amine # PATENT # Substituted quinoxaline derivatives # WO2016180537A1
Selvita_Example_62 # N-(4-methanesulfonylpyridin-3-yl)-8-(3-methoxyphenyl)quinoxalin-6-amine # PATENT #
Substituted quinoxaline derivatives # WO2016180537A1
Selvita_Example_61 # 8-(1H-1,3-benzodiazol-6-yl)-N-(4-methanesulfonylpyridin-3-yl)quinoxalin-6-amine # PATENT #
Substituted quinoxaline derivatives # WO2016180537A1
Selvita_Example_60 # 8-(4-ethylphenyl)-N-(4-methanesulfonylpyridin-3-yl)quinoxalin-6-amine # PATENT # Substituted
quinoxaline derivatives # WO2016180537A1
Selvita_Example_59 # 8-(7-fluoro-1-methyl-1H-indol-6-yl)-N-(4-methanesulfonylpyridin-3-yl)quinoxalin-6-amine #
PATENT # Substituted quinoxaline derivatives # WO2016180537A1
Selvita_Example_58 # 8-[3-(chloromethyl)-1-benzofuran-5-yl]-N-(4-methanesulfonylpyridin-3-yl)quinoxalin-6-amine #
PATENT # Substituted quinoxaline derivatives # WO2016180537A1
Selvita_Example_57 # 4-methanesulfonyl-N1-methyl-N3-[8-(1-methyl-1H-indol-6-yl)quinoxalin-6-yl]benzene-1,3-diamine
# PATENT # Substituted quinoxaline derivatives # WO2016180537A1
Selvita_Example_56 # 3-{[8-(1-methyl-1H-indol-6-yl)quinoxalin-6-yl]amino}-N-[(1-methyl-1H-pyrazol-4-yl)methyl]pyridine-
4-carboxamide # PATENT # Substituted quinoxaline derivatives # WO2016180537A1
Selvita_Example_55 # 3-{[8-(1-methyl-1H-indol-6-yl)quinoxalin-6-yl]amino}-N-[(pyrimidin-5-yl)methyl]pyridine-4-
carboxamide # PATENT # Substituted quinoxaline derivatives # WO2016180537A1
Selvita_Example_54 # 3-{[8-(1-methyl-1H-indol-6-yl)quinoxalin-6-yl]amino}-N-(pyrimidin-5-yl)pyridine-4-carboxamide #
PATENT # Substituted quinoxaline derivatives # WO2016180537A1
Selvita_Example_53 # N,N-dimethyl-3-{[8-(1-methyl-1H-indol-6-yl)quinoxalin-6-yl]amino}pyridine-4-carboxamide #
PATENT # Substituted quinoxaline derivatives # WO2016180537A1
Selvita_Example_52 # N-methyl-3-{[8-(1-methyl-1H-indol-6-yl)quinoxalin-6-yl]amino}pyridine-4-carboxamide # PATENT #
Substituted quinoxaline derivatives # WO2016180537A1
Selvita_Example_51 # N-(4-methanesulfonylpyridin-3-yl)-8-(3-methylphenyl)quinoxalin-6-amine # PATENT # Substituted
quinoxaline derivatives # WO2016180537A1
Selvita_Example_50 # 8-[3-(dimethylamino)phenyl]-N-(4-methanesulfonylpyridin-3-yl)quinoxalin-6-amine # PATENT #
Substituted quinoxaline derivatives # WO2016180537A1
Selvita_Example_49 # N-(4-methanesulfonylpyridin-3-yl)-8-[1-(propan-2-yl)-1H-indol-6-yl]quinoxalin-6-amine # PATENT
# Substituted quinoxaline derivatives # WO2016180537A1
Selvita_Example_48 # N-(4-methanesulfonylpyridin-3-yl)-8-[3-(2H-1,2,3-triazol-4-yl)phenyl]quinoxalin-6-amine # PATENT
# Substituted quinoxaline derivatives # WO2016180537A1
Selvita_Example_47 # 3-{[8-(1-methyl-1H-indol-6-yl)quinoxalin-6-yl]oxy}pyridine-4-carbonitrile # PATENT # Substituted
quinoxaline derivatives # WO2016180537A1
Selvita_Example_46 # 1-[4-(4-methanesulfonyl-3-{[8-(1-methyl-1H-indol-6-yl)quinoxalin-6-yl]amino}phenyl)piperazin-1-
yl]ethan-1-one # PATENT # Substituted quinoxaline derivatives # WO2016180537A1
Selvita_Example_45 # N-[2-methanesulfonyl-5-(4-methylpiperazin-1-yl)phenyl]-8-(1-methyl-1H-indol-6-yl)quinoxalin-6-
amine # PATENT # Substituted quinoxaline derivatives # WO2016180537A1
Selvita_Example_44 # 4-methanesulfonyl-3-{[8-(1-methyl-1H-indol-6-yl)quinoxalin-6-yl]amino}pyridin-1-ium-1-olate #
PATENT # Substituted quinoxaline derivatives # WO2016180537A1
Selvita_Example_43 # N-[2-methanesulfonyl-5-(2H-1,2,3,4-tetrazol-5-yl)phenyl]-8-(1-methyl-1H-indol-6-yl)quinoxalin-6-
amine # PATENT # Substituted quinoxaline derivatives # WO2016180537A1
Selvita_Example_42 # N-[5-(1H-imidazol-1-yl)-2-methanesulfonylphenyl]-8-(1-methyl-1H-indol-6-yl)quinoxalin-6-amine #
PATENT # Substituted quinoxaline derivatives # WO2016180537A1
Selvita_Example_41 # N-(4-methanesulfonyl-3-{[8-(1-methyl-1H-indol-6-yl)quinoxalin-6-yl]amino}phenyl)acetamide #
PATENT # Substituted quinoxaline derivatives # WO2016180537A1
Selvita_Example_40 # 8-(1-methyl-1H-indol-6-yl)-N-[4-(4-methylpiperazin-1-yl)pyridin-3-yl]quinoxalin-6-amine # PATENT
# Substituted quinoxaline derivatives # WO2016180537A1
Selvita_Example_39 # 8-(2,5-dimethylphenyl)-N-(4-methanesulfonylpyridin-3-yl)quinoxalin-6-amine # PATENT #
Substituted quinoxaline derivatives # WO2016180537A1
Selvita_Example_38 # N-[5-(aminomethyl)-2-methanesulfonylphenyl]-8-(1-methyl-1H-indol-5-yl)quinoxalin-6-amine #
PATENT # Substituted quinoxaline derivatives # WO2016180537A1
Selvita_Example_37 # 8-(2,3-dihydro-1-benzofuran-5-yl)-N-(4-methanesulfonylpyridin-3-yl)quinoxalin-6-amine # PATENT
# Substituted quinoxaline derivatives # WO2016180537A1
Selvita_Example_36 # 6-methanesulfonyl-N1-[8-(1-methyl-1H-indol-6-yl)quinoxalin-6-yl]benzene-1,3-diamine # PATENT
# Substituted quinoxaline derivatives # WO2016180537A1
Selvita_Example_35 # N-(2-methanesulfonyl-5-nitrophenyl)-8-(1-methyl-1H-indol-6-yl)quinoxalin-6-amine # PATENT #
Substituted quinoxaline derivatives # WO2016180537A1
Selvita_Example_34 # 5-(1-methyl-1H-indol-5-yl)-7-{1H,2H,3H-pyrrolo[2,3-c]pyridin-1-yl]quinoxaline # PATENT #
Substituted quinoxaline derivatives # WO2016180537A1
Selvita_Example_33 # 8-(1-methyl-1H-indol-5-yl)-N-[4-(pyrimidin-5-yl)pyridin-3-yl]quinoxalin-6-amine # PATENT #
Substituted quinoxaline derivatives # WO2016180537A1
Selvita_Example_32 # 8-(1-methyl-1H-indol-5-yl)-N-[4-(4-methylpiperazin-1-yl)pyridin-3-yl]quinoxalin-6-amine # PATENT
# Substituted quinoxaline derivatives # WO2016180537A1
Selvita_Example_31 # 8-(1-methyl-1H-indol-5-yl)-N-[4-(1-methyl-1H-pyrazol-4-yl)pyridin-3-yl]quinoxalin-6-amine #
PATENT # Substituted quinoxaline derivatives # WO2016180537A1
Selvita_Example_30 # N-(4-chloropyridin-3-yl)-8-(1-methyl-1H-indol-5-yl)quinoxalin-6-amine # PATENT # Substituted
quinoxaline derivatives # WO2016180537A1
Selvita_Example_29 # 3-{[8-(1-methyl-1H-indol-5-yl)quinoxalin-6-yl]amino}pyridine-4-carboxamide # PATENT #
Substituted quinoxaline derivatives # WO2016180537A1
Selvita_Example_28 # 3-{[8-(1-methyl-1H-indol-5-yl)quinoxalin-6-yl]amino}pyridine-4-carbonitrile # PATENT # Substituted
quinoxaline derivatives # WO2016180537A1
Selvita_Example_27 # N-(5-methanesulfonylpyrimidin-4-yl)-8-(1-methyl-1H-indol-6-yl)quinoxalin-6-amine # PATENT #
Substituted quinoxaline derivatives # WO2016180537A1
Selvita_Example_26 # 3-{[8-(3-methyl-1-benzofuran-5-yl)quinoxalin-6-yl]amino}pyridine-4-carboxamide # PATENT #
Substituted quinoxaline derivatives # WO2016180537A1
Selvita_Example_25 # 4-methanesulfonyl-3-{[8-(1-methyl-1H-indol-6-yl)quinoxalin-6-yl]amino}benzonitrile # PATENT #
Substituted quinoxaline derivatives # WO2016180537A1
Selvita_Example_24 # 3-{[8-(3-methyl-1-benzofuran-5-yl)quinoxalin-6-yl]amino}pyridine-4-carbonitrile # PATENT #
Substituted quinoxaline derivatives # WO2016180537A1
Selvita_Example_23 # N-(4-methoxypyridin-3-yl)-8-(1-methyl-1H-indol-6-yl)quinoxalin-6-amine # PATENT # Substituted
quinoxaline derivatives # WO2016180537A1
Selvita_Example_22 # N-(4-methanesulfonylpyridin-3-yl)-8-(3-methyl-1-benzofuran-5-yl)quinoxalin-6-amine # PATENT #
Substituted quinoxaline derivatives # WO2016180537A1
Selvita_Example_21 # N-(2-methanesulfonylphenyl)-8-{1-methyl-1H-pyrrolo[2,3-b]pyridin-6-yl}quinoxalin-6-amine #
PATENT # Substituted quinoxaline derivatives # WO2016180537A1
Selvita_Example_20 # N,N-dimethyl-2-{[8-(1-methyl-1H-indol-6-yl)quinoxalin-6-yl]amino}benzene-1-sulfonamide #
PATENT # Substituted quinoxaline derivatives # WO2016180537A1
Selvita_Example_19 # 3-{[8-(1-methyl-1H-indol-6-yl)quinoxalin-6-yl]amino}pyridine-4-carboxamide # PATENT #
Substituted quinoxaline derivatives # WO2016180537A1
Selvita_Example_18 # 3-{[8-(1-methyl-1H-indol-6-yl)quinoxalin-6-yl]amino}pyridine-4-carbonitrile # PATENT # Substituted
quinoxaline derivatives # WO2016180537A1
Selvita_Example_17 # N3-[8-(1-methyl-1H-indol-6-yl)quinoxalin-6-yl]pyridine-2,3-diamine # PATENT # Substituted
quinoxaline derivatives # WO2016180537A1
Selvita_Example_16 # N-methyl-2-{[8-(1-methyl-1H-indol-6-yl)quinoxalin-6-yl]amino}benzene-1-sulfonamide # PATENT #
Substituted quinoxaline derivatives # WO2016180537A1
Selvita_Example_15 # 8-(1-methyl-1H-indol-6-yl)-N-[2-(piperazine-1-sulfonyl)phenyl]quinoxalin-6-amine # PATENT #
Substituted quinoxaline derivatives # WO2016180537A1
Selvita_Example_14 # 3-{[8-(1-methyl-1H-indol-6-yl)quinoxalin-6-yl]amino}-1,2-dihydropyridin-2-one # PATENT #
Substituted quinoxaline derivatives # WO2016180537A1
Selvita_Example_13 # N-(2-methoxypyridin-3-yl)-8-(1-methyl-1H-indol-6-yl)quinoxalin-6-amine # PATENT # Substituted
quinoxaline derivatives # WO2016180537A1
Selvita_Example_11 # N-(4-methanesulfonylpyridin-3-yl)-8-(1-methyl-1H-indol-6-yl)quinoxalin-6-amine # PATENT #
Substituted quinoxaline derivatives # WO2016180537A1
Selvita_Example_10 # N-(5-bromo-2-methanesulfonylphenyl)-8-(1-methyl-1H-indol-6-yl)quinoxalin-6-amine # PATENT #
Substituted quinoxaline derivatives # WO2016180537A1
Selvita_Example_9 # 8-(1,3-benzothiazol-5-yl)-N-(4-methanesulfonylpyridin-3-yl)quinoxalin-6-amine # PATENT #
Substituted quinoxaline derivatives # WO2016180537A1
Selvita_Example_8 # 2-{[8-(1-methyl-1H-indol-6-yl)quinoxalin-6-yl]amino}benzene-1-sulfonamide # PATENT #
Substituted quinoxaline derivatives # WO2016180537A1
Selvita_Example_7 # 8-(1-methyl-1H-indol-6-yl)-N-[2-(morpholine-4-sulfonyl)phenyl]quinoxalin-6-amine # PATENT #
Substituted quinoxaline derivatives # WO2016180537A1
Selvita_Example_6 # N-(2-methanesulfonylpyridin-3-yl)-8-(1-methyl-1H-indol-6-yl)quinoxalin-6-amine # PATENT #
Substituted quinoxaline derivatives # WO2016180537A1
Selvita_Example_5 # 8-(2-chloro-5-methoxyphenyl)-N-(4-methanesulfonylpyridin-3-yl)quinoxalin-6-amine # PATENT #
Substituted quinoxaline derivatives # WO2016180537A1
Selvita_Example_4 # 8-(1,3-benzothiazol-6-yl)-N-(4-methanesulfonylpyridin-3-yl)quinoxalin-6-amine # PATENT #
Substituted quinoxaline derivatives # WO2016180537A1
Selvita_Example_3 # N-(2-methanesulfonylphenyl)-8-(1-methyl-1H-indol-6-yl)quinoxalin-6-amine # PATENT #
Substituted quinoxaline derivatives # WO2016180537A1
Selvita_Example_2 # 5-(1-methyl-1H-indol-6-yl)-7-{1H,2H,3H-pyrrolo[2,3-c]pyridin-1-yl}quinoxaline # PATENT #
Substituted quinoxaline derivatives # WO2016180537A1
Selvita_Example_1 # 8-(2,3-dihydro-1,4-benzodioxin-6-yl)-N-(4-methanesulfonylpyridin-3-yl)quinoxalin-6-amine #
PATENT # Substituted quinoxaline derivatives # WO2016180537A1

### Example ADD-1

AZ67 in Batten Disease

Methods

### AZ67 In Vivo Administration

AZ67 for in vivo usage was dissolved in 20% (wt/vol) PEG200 in PBS to a 20 mM concentration. 4 groups were generated (4-6 animals/group), namely: WT-vehicle, CLN7Δex2-vehicle, WT-AZ67, CLN7Δex2-AZ67. The canula was inserted intracerebroventricularly at the age of 8 weeks and, after at least of 15 days of recovery, we injected the AZ67 at the dose identified previously (l nmol/mouse) every 24 h. The duration of the experiment was determined by the presence of hindlimb clasping the CLN7Δex2-control (vehicle), being this time two months. After this, the animals were perfused, and their brains dissected to be investigated by immunofluorescence and electron microscopy (EM).

### Mouse Perfusion and Immunohistochemistry

Mice (5 months for AZ67 intraventricular injections; 3 months for mCAT expression approach) were anaesthetized by intraperitoneal injection of a mixture of xylazine hydrochloride (Rompun; Bayer) and ketamine hydrochloride/chlorbutol (Imalgene; Merial) (1:4) at 1 ml per kg body weight and then perfused intra-aortically with 0.9% NaCl followed by 5 ml p/g body weight of Somogyi (4% (wt/vol) paraformaldehyde, and 0.2% (vol/vol) picric acid, in 0.1 M PB; pH 7.4). After perfusion, brains were dissected out sagitally in two parts and post-fixed with Somogyi for 2 h at room temperature. Brain blocks were rinsed successively for 10 min, 30 min and 2 h with 0.1 M PB solution and cryoprotected in 10%, 20% and 30% (wt/vol) sucrose in PB sequentially, until they sank. After cryoprotection, 40-m-thick sagittal sections were obtained with a freezing-sliding cryostat (Leica). Sectioning of WT and Cln7Δex2 brains were performed under the same conditions and sessions. The sections were collected serially in a 12-well plate in 0.1 M PB, rinsed three times for 10 min in 0.1 M PB and used for subsequent immunohistochemistry and lipofuscin observation. The section-containing wells that were not used were kept in freezer mix (30% (vol/vol) polyethylene glycol, 30% (vol/vol) glycerol in 0.1 M PB) at −20° C. For immunohistochemistry, sections were incubated sequentially in (i) 5 mg/ml sodium borohydride in PB for 30 min (to remove aldehyde autofluorescence); (ii) three PBS washes of 10 min each; (iii) 1/500 anti-GFAP (G6171; Sigma) and 1/500 anti-IBA1 (019-19741; Wako) or 1/500 anti-ATP-C(SCMAS) (ab181243; Abcam) in 0.02% Triton X-100 (Sigma) and 5% goat serum (Jackson Immuno-Research) in 0.1 M PB for 72 h at 4° C.; (iv) three PB washes of 10 min each; (v) fluorophore conjugated secondary antibodies, 1/500 Cy2 goat anti-mouse and 1/500 Cy3 goat anti-rabbit (Jackson Immuno-Research) or Alexa-488 (A11008; Molecular Probes) in PB for 2 h at room temperature; and (vi) 0.5 μg/ml DAPI in PB for 10 min at room temperature. After being rinsed with PB, sections were mounted with Fluoromount (Sigma) aqueous mounting medium and cover slips (Thermo Fisher)₅₁, For autofluorescence (lipofuscine accumulation) sections were mounted directly.

### Imaging and Quantification

Sections were examined with epifluorescence and the appropriate filter sets under an Operetta CLS high-content imaging system (PerkinElmer). Large fields of view were acquired with an 5× scan using an OperaPHX/OPRTCLS 5× Air Objective. Then high-resolution images were acquired using an OperaPHX/OPRTCLS Air Objective 20× hNA objective. Immunohistochemical digital images were used to analyse different proteins staining in the three most sagittal sections per animal from, at least, two different animals per condition (n=2). Images were analysed with the Harmony software with PhenoLOGIC (PerkinElmer). Interest brain areas (cortex, hippocampus and cerebellum) were selected and subsequently quantified as mean intensity per area by using the ‘measure rectangle’ function, which represents the mean intensity of a channel per selected area.

Results

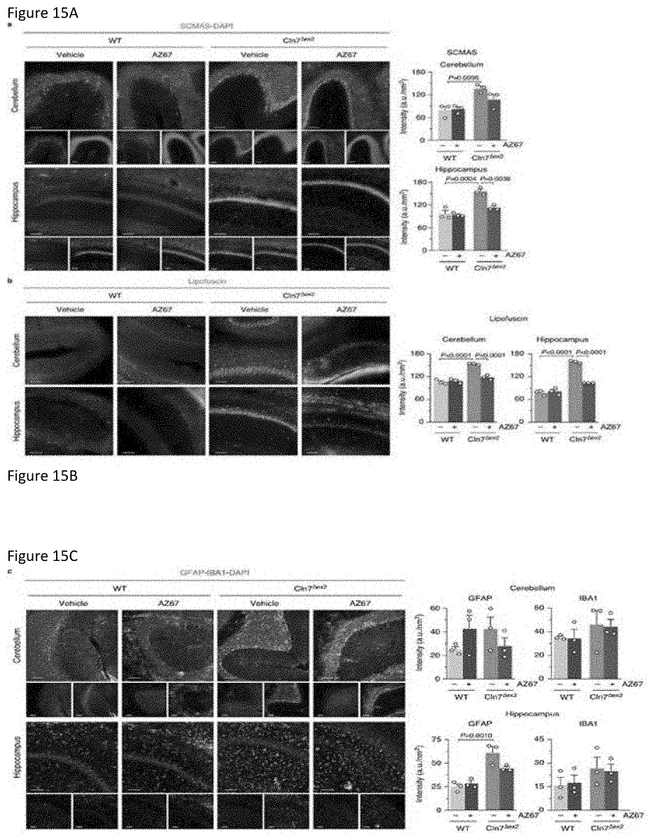

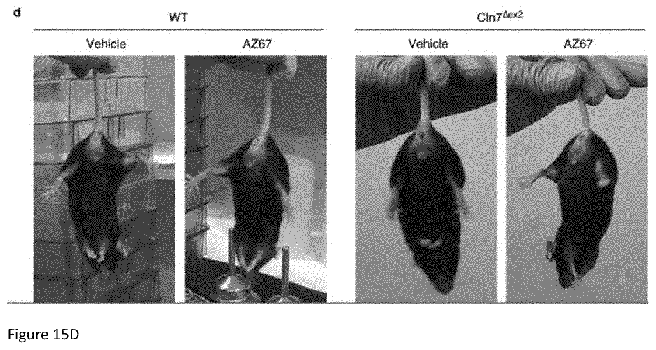

In vivo, AZ67 prevented the accumulation of SCMAS, lipofuscin and reactive astroglia in the cortex (see FIGS. 15A-15C), of the Cln7Δex2 mice. Hindlimb paralysis in Cln7Δex2 mice was prevented by AZ67 (see FIG. 15D), indicating functional recovery.

Effect of PPFKFB3 inhibitor in patient-derived neural precursor cells

Methods

Induced Pluripotent Stem Cells (iPSC) and Neural Progenitor Cells (NPC) Generation

iPSC were generated from two CLN7 patients (Pa380 and Pa474). then characterized and differentiated to NPC as previously described₃₉. Human iPSC-derived NPCs from a control patient and patients Pa380 (c.881C>A; pT294K) and Pa474 (c.1393C>T; p.R465 W) harboring the indicated CLN7 homozygous mutations. were plated on Matrigel® Matrix in Nunc™ Lab-Tek™ S-well Chamber Slides and cultured in Neural Expansion Medium (NEM) with DMEM/F12, NEAA, N-2 supplement, B-27 supplement, heparin, bFGF protein, penicillin/strepromycin. iPSCs pluripotency was confirmed by immunocytochemistry using OCT4 (1:200, ab19857: Abcam), SOX2 (1:100, AF2018; R&D Systems), Nanog (t:100, ab21624: Abcam) and Tra-1-60 (1:200, MAB1295: R&D Systems), and by confirming their ability to differentiate into neurons using TUJ-1 (1:200, MAB1195; R&D Systems) staining. NPC identity was confirmed by Nestini+/SOX2− immunostaining.

### NPC Immunocytochemistry

NPCs were fixed with 100% iced-cold methanol for 5 min and incubated in blocking solution (1% (v/v) normal goat serum, 0.1% (w/v) bovine serum albumin (BSA), 0.1% (v/v) Triton X-100 in DPBS). The antibodies were incubated in blocking solution. The incubation of the primary antibody (mouse α-ATP5A. 1:100, Abeam, ab14748) or SCMAS (11200, ab181243, Abcam) was performed for 2 h at room temperature, and the secondary antibodies (Alexa Fluor 568 goat α-mouse, 1:500, Invitrogen or Alexa Fluor 488 goat anti-rabbit, A-I1008, Thermo) were applied for 1 h at room temperature. Slides were mounted with VECTASHIELD Mounting Medium with DAPI, incubated for 24 h at 4° C. and imaged with a Zeiss Axio Imager M2 fluorescence microscope or under an inverted microscope (Nikon; Eclipse Ti-E) equipped with a pre-centred fibre illuminator (Nikon; Intensilight C-HGFI), B/W CCD digital camera (Hamamatsu; ORCA-E.R.). Fluorescence quantification was performed. after appropriate thresholding using the ImageJ software (NIH). The pixel intensity profile of ATP5A immunodecoration was analyzed across the maximal axis of the cell that departs from the nucleus, using the plot profile plugin of ImageJ software. A representative profile is shown for each condition.

Results

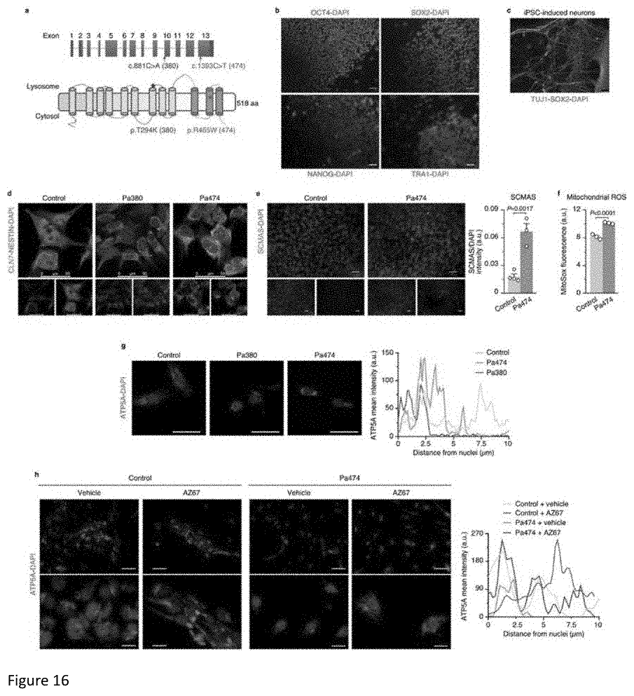

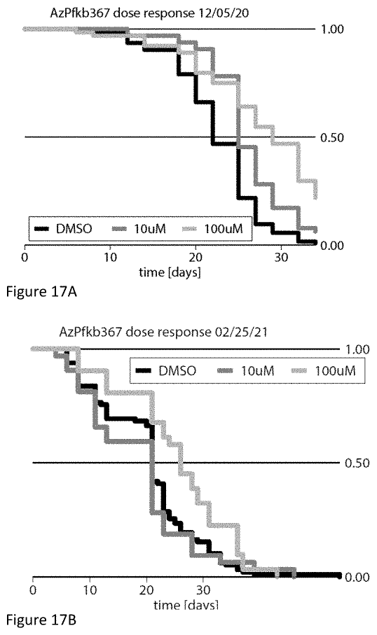

FIG. 16 shows that AZ67 rectified the loss of perinuclear mitochondria observed in induced pluripotent stems cells (iPSC)-derived neural precursor cells (NPC) from CLN7 patients.

### Example ADD-2

Extending lifespan of C. elegance by PFKB3 Inhibitor

The test of AZ67 compound had been made according to protocol disclosed in Solis, G. M., Petrascheck, M. Measuring Caenorhabditis elegans

Life Span in 96 Well Microtiter Plates.J.Vis.Exp. (49), e2496, doi:10.3791/2496 (2011) FIGS. 17A and 17B show AzPfkb367 dose responses.

| | | | | | | Lifespan, | | | | |
|---|---|---|---|---|---|---|---|---|---|---|
| vnu | strain | Drug | conc | % LS change | P_value | days | N0 | set | add | date |
| 5 | N2 | 8DMSO | 0 | | | 22.54 | 124 | Aib | 0 | 11521 |
| 7 | N2 | AzPfkb367 | 10 | 15 | 1.63E−05 | 25.98 | 64 | Aib | 0 | 11521 |
| 9 | N2 | AzPfkb367 | 100 | 22 | 6.53E−11 | 27.47 | 64 | Aib | 0 | 11521 |

| | | | Conc | | | Lifespan, | | | | |
|---|---|---|---|---|---|---|---|---|---|---|
| vnu | strain | Drug | (uM) | % LS change | P_value | days | N0 | set | add | date |
| 4 | N2 | 8DMSO | 0 | | | 20.46 | 98 | AI | 0 | 120520 |
| 6 | N2 | AzPfkb367 | 10 | −8 | 0.552067 | 18.81 | 32 | AI | 0 | 120520 |
| 8 | N2 | AzPfkb367 | 100 | 24 | 0.007886 | 25.45 | 31 | AI | 0 | 120520 |

NO—number of animals,
conc—concentration,
% LS change—Life Span change

1. A compound of Formula (0): 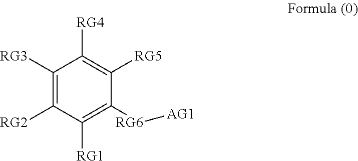
or a pharmaceutically acceptable salt thereof, or N-oxide, or solvate, or tautomer, or stereoisomer, or racemate, including mixtures thereof in all ratios, wherein RG6 and RG5 is one of the following: A) RG6 and RG5 are taken together with the N to which they are attached to form a C2-C8 heterocycloalkyl optionally substituted with one or more substituents;
RG1, RG3 and RG4 are independently selected from R_{M}; RG2 is R_{L}; RG5 is Z; RG6 is —C(═O)—; AG1 is —Ar_{C}—Ar_{T};
thus the Formula (0) can be represented as the Formula (I): 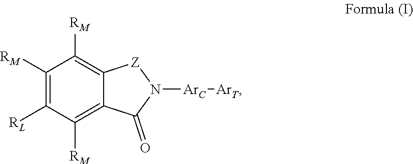
wherein: Z is selected from —C(═O)— and —C(R^{a})(R^{b})—;
R^{a} and R^{b} are independently selected from hydrogen, hydroxyl, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;
wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR¹R², —OH, aryl, heteroaryl, C₁-C₆alkyl, C₁-C₆ alkoxy, C₃-C₈ cycloalkyl, —O—C₃-C₈ cycloalkyl, C₂-C₈ heterocycloalkyl, and -O—C₂-C₈ heterocycloalkyl;
Ar_{c} is selected from C₃-C₈ cycloalkenylene, C₂-C₈ heterocycloalkenylene, arylene, and heteroarylene; wherein Ar_{c} is substituted with one or more R_{C};
each R_{C} are independently selected from —CN, —OH, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, optionally substituted —O—C₂-C₈ heterocycloalkyl, optionally substituted heteroaryl, optionally substituted aryl, —C(═O)OH, —C(═O)OR³, —C(═O)NR⁴R⁵, —S(═O)₂NR⁴R⁵, —NHC(═O)H, —NHC(═O)R⁶, —NHS(═O)₂R⁶, and —C(═O)NHS(═O)₂R⁶;
wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, —O—C₃-C₈ cycloalkyl, C₂-C₈ heterocycloalkyl, —O—C₂-C₈ heterocycloalkyl, aryl, and heteroaryl are optionally substituted with one or more substituents independently selected from —OH, halogen, —C(═O)OR⁷, —C(═O)R⁶, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR⁷R⁸;
each R¹ and R² is independently selected from hydrogen, optionally substituted C₁-C₆ alkyl, and optionally substituted C₃-C₅ cycloalkyl;
wherein the C₁-C₆ alkyl is optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl is optionally substituted with one or more substituents independently selected from halogen, —C(═O)OR⁷, —C(═O)NR⁷R⁸, —OH, aryl, heteroaryl, C₁-C₆ alkyl, C₁-C₆ alkoxy, C₃-C₈ cycloalkyl, —O—C₃-C₈ cycloalkyl, C₂-C₈ heterocycloalkyl, and -O—C₂-C₈ heterocycloalkyl;
or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;
each R³ is independently C₁-C₆ alkyl optionally substituted with one or more substituents independently selected from halogen, —OH, optionally substituted —OC(═O)C₁-C₆alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;
wherein the —OC(═O)C₁-C₆ alkyl and —C(═O)O—C₁-C₆ alkyl are optionally substituted with one or more substituents independently selected from halogen, —OH, and —NR⁷R⁸; and
wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
or each R³ is independently C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;
wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and
wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
each R⁴ and R⁵ is independently selected from hydrogen, optionally substituted C₁-C₆ alkyl, optionally substituted C₃-C₈ cycloalkyl, optionally substituted aryl, and optionally substituted heteroaryl;
wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;
each R⁶ are independently selected from optionally substituted C₁-C₆ alkyl, optionally substituted C₃-C₈ cycloalkyl, optionally substituted aryl, and optionally substituted heteroaryl;
wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
each R⁷ and R⁸ is independently selected from hydrogen and C₁-C₆ alkyl;
or R⁷ and R⁸ are taken together with the N to which they are attached to form a C₂-C₈ heterocycloalkyl optionally substituted with one or more C₁-C₆ alkyl substituents;
Ar_{T} is selected from pyridinyl, pyrimidinyl, pyridazinyl, pyrazinyl, phenyl, pyrrolyl, furanyl, thiophenyl, pyrazolyl, imidazolyl, triazolyl, and tetrazolyl, wherein Ar_{T} is optionally substituted by one or more substituents independently selected from halogen, —OH, —NR⁷R⁸, —CN, optionally substituted C₁-C₆ alkyl, optionally substituted C₁-C₆ alkoxy, optionally substituted C₃-C₅ cycloalkyl, optionally substituted —O—C₃-C₈
cycloalkyl, optionally substituted C₂-C₈ heterocycloalkyl, and optionally substituted —O—C₂-C₈ heterocycloalkyl;
wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and -O—C₂-C₈ heterocycloalkyl;
each R_{M} is independently selected from hydrogen, halogen, —OH, —CN, optionally substituted C₁-C₆ alkyl, optionally substituted C₁-C₆ alkoxy, optionally substituted C₃-C₈ cycloalkyl, optionally substituted —O—C₃-C₅ cycloalkyl, optionally substituted C₂-C₅ heterocycloalkyl, and optionally substituted —O—C₂-C₅ heterocycloalkyl;
wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and -O—C₂-C₈ heterocycloalkyl;
R_{L} is selected from —OH, —CN, optionally substituted C₁-C₆ hydroxyalkyl, optionally substituted C₁-C₆ alkoxy, optionally substituted heteroaryl, —C(═O)OH, —C(═O)OR⁹, —C(═O)NR¹⁰R¹¹, —S(═O)₂NR¹⁰R¹¹, —NHC(═O)H, —NHC(═O)R¹², —NHS(═O)₂R¹² and —C(═O)NHS(═O)₂R¹²;
wherein the C₁-C₆ hydroxyalkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the heteroaryl is optionally substituted with one or more of —OH, —O—C(═O)C₁-C₆ alkyl, (C₁-C₄ alkylene)-O—C(═O)C₁-C₆ alkyl, C₁-C₆ alkyl-(aryl), C₁-C₆ alkyl-(heteroaryl), halogen, —C(═O)OR⁷, —C(═O)R¹², —C(═O)NR¹R², C₁-C₆ alkyl, C₁-C₆ hydroxyalkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR¹R²;
R⁹ is C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;
wherein the —OC(═O)C₁-C₆ alkyl and —C(═O)O—C₁-C₆ alkyl are optionally substituted with one or more substituents independently selected from halogen, —OH, and —NR⁷R⁸; and
wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
or R⁹ is C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;
wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and
wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
each R¹⁰ and R¹¹ is independently selected from hydrogen, optionally substituted C₁-C₆alkyl, optionally substituted C₃-C₈ cycloalkyl, optionally substituted aryl, and optionally substituted heteroaryl;
wherein the C₁-C₆ alkyl is optionally substituted with one or more substituents independently selected from halogen, —C(═O)OR⁷, —C(═O)NR¹R², —OH, aryl (optionally substituted with —OH, halogen, —C(═O)OR⁷, —C(═O)NR⁷R⁸, C₁-C₆ alkyl, C₁-C₆ alkoxy, —NR⁷R⁸), and heteroaryl (optionally substituted with —OH, halogen, —C(═O)OR⁷, —C(═O)NR⁷R⁸, C₁-C₆ alkyl, C₁-C₆ alkoxy, —NR⁷R⁸, C₃-C₈ cycloalkyl, —O—C₃-C₈ cycloalkyl, C₂-C₈ heterocycloalkyl, or —O—C₂-C₈ heterocycloalkyl); and
wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
or R¹⁰ and R¹¹ are taken together with the N to which they are attached to form a C₂-C₈ heterocycloalkyl optionally substituted with one or more substituents independently selected from halogen, —OH, C₁-C₆ alkyl, and C₁-C₆ alkoxy;
R¹² is selected from optionally substituted C₁-C₆ alkyl, optionally substituted C₃-C₈ cycloalkyl, optionally substituted aryl, and optionally substituted heteroaryl;
wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
provided that:
(a) at least one of R_{C} is not —NHCOR⁶ when R_{L} is —NHCOR¹² and Ar_{C} is heterocycloalkenylene or heteroarylene; or
(b) at least one of R_{C} is not -Me when R_{L} is —OMe; or
(c) at least one of R_{C} is not —OEt when R_{L} is —C(═O)OH; or
(d) at least one of R_{C} is not —OH when R_{L} is —C(═O)OH; or
(e) at least one of R_{C} is not -Me when R_{L} is —C(═O)OH; or
(f) at least one of R_{C} is not -Et when R_{L} is —OMe; or
(g) at least one of R_{C} is not optionally substituted benzoxazolyl when R_{L} is —C(═O)OH; or
(h) at least one of R_{C} is not optionally substituted isoindoline-1,3-dione when R_{L} is —C(═O)OH;
B) RG6 and RG5 do not form a C₂-C₈ heterocycloalkyl; RG1 is R⁵; RG2 is R¹; RG3 is R⁶; RG4 is R²⁰; RG5 is R⁴; RG6 is R¹⁰; AG1 is A;
thus the Formula (0) can be represented as the Formula (VII): 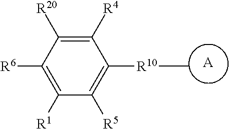
Formula (VII), or a pharmaceutically acceptable salt thereof, wherein:
A is selected from: 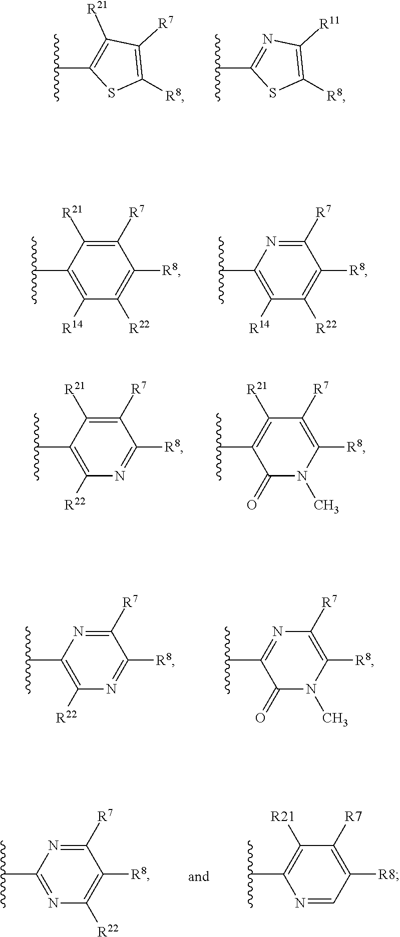
R¹ is selected from hydrogen, halogen, hydroxyl, C₁-C₆ alkyl, and C₁-C₆ alkoxy,
wherein the C₁-C₆ alkyl and C₁-C₆ alkoxy are optionally substituted with one or more halogens;
each R² and R³ is independently selected from hydrogen and C₁-C₆ alkyl,
wherein the C₁-C₆ alkyl is optionally substituted with one or more halogens;
or R² and R³ are taken together with the N to which they are attached to form a 3- to 10-membered heterocycle optionally substituted with one or more substituents independently selected from C₁-C₆ alkyl;
R⁴ is selected from hydrogen, halogen, C₁-C₆ alkyl, and C₁-C₆ alkoxy,
wherein the C₁-C₆ alkyl and C₁-C₆ alkoxy are optionally substituted with one or more substituents independently selected from halogens;
R⁸ is selected from —C(═O)OR¹⁵, —C(═O)NR²R³, —S(═O)₂NR²R³, —C(═O)NHR¹⁵, —CH₂OH, 3-hydroxyoxetan-3-yl, and —NH₂;
R⁶ is selected from hydrogen, halogen, hydroxyl, 5-membered heteroaryl, C₁-C₆ alkyl, —C(═O)OR¹⁵, —C(═O)R¹², —C(═O)NHR¹⁵, and —C(═O)N═S(═X³)(CH₃)₂,
wherein the C₁-C₆ alkyl are optionally substituted with one or more R⁹, and
wherein 5-membered heteroaryl contains at least two heteroatoms and is optionally substituted with one or more substituents independently selected from R¹⁷;
R⁷ is selected from hydrogen, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,
wherein the C₁-C₆ alkyl and C₁-C₆ alkoxy are optionally substituted with one or more halogens, and
wherein C₃-C₈ cycloalkyl, —O—C₃-C₈ cycloalkyl, 3- to 10-membered heterocycloalkyl, —O-(3- to 10-membered heterocycloalkyl), aryl and heteroaryl are optionally substituted with one or more R²⁴;
R⁸ is selected from hydrogen, —NO₂, C₁-C₆ alkyl, aryl, and heteroaryl,
wherein the C₁-C₆ alkyl and C₁-C₆ alkoxy are optionally substituted with one or more substituents independently selected at each occurrence from halogen; and
wherein aryl and heteroaryl are optionally substituted with one or more substituents independently selected at each occurrence from R²³;
or R⁷ and R⁸ are taken together to form a C₅-C₁₀ carbocycle or 5- to 10-membered heterocycle,
wherein C₅-C₁₀ carbocycle and 5- to 10-membered heterocycle are optionally substituted with one or more substituents independently selected from halogen, hydroxyl, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,
wherein the C₁-C₆ alkyl and C₁-C₆ alkoxy are optionally substituted with one or more halogens, and
wherein aryl, heteroaryl, C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;
each R⁹ is independently selected from hydroxy and —COOH;
R¹⁰ is selected from —C(═O)—X¹—, —CH₂—X¹—, —X¹—C(═O)—, and —X¹-CH₂—;
R¹¹ is selected from hydrogen, —NO₂, C₁-C₆ alkyl, C₁-C₆ alkoxy, C₃-C₈ cycloalkyl, —O—C₃-C₈ cycloalkyl, 3- to 10-membered heterocycloalkyl, and —O—(3- to 10-membered heterocycloalkyl),
wherein the C₁-C₆ alkyl and C₁-C₆ alkoxy are optionally substituted with one or more halogens, and
wherein C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;
R¹² is selected from alanine, arginine, asparagine, aspartic acid, asparagine, aspartic acid, cysteine, glutamic acid, glutamine, glutamine, glutamic acid, glycine, histidine, isoleucine, leucine, lysine, methionine, phenylalanine, proline, serine, threonine, tryptophan, tyrosine, and valine, wherein the point of attachment of R¹² is a nitrogen atom;
R¹⁴ is selected from hydrogen, halogen, hydroxyl, nitrile, —C(═O)CR¹⁵ and —C(═O)OR¹⁵;
each R¹⁵ is independently selected from hydrogen and C₁-C₆ alkyl, -heterocyclyl,
wherein the C₁-C₆ alkyl is optionally substituted with one or more substituents independently selected at each occurrence from —C(═O)NR²R³, -heterocyclyl, —NR²R³;
wherein the heterocyclyl is optionally substituted with one or more substituents independently selected at each occurrence from R² and R³,
R¹⁷ is selected from C₁-C₆ alkyl, aryl, and 6-membered heteroaryl,
wherein the C₁-C₆ alkyl is optionally substituted with one or more hydroxy, and
wherein aryl and 6-membered heteroaryl are optionally substituted with one or more substituents independently selected from halogen, —R², and —OR²;
R²⁰ is selected from hydrogen, halogen, hydroxyl, —COOH, —NC(═O)R², —OR², 5-membered heteroaryl, C₁-C₆ alkyl, —C(═O)N═S(═X³)(CH₃)₂, —CH₂(OH)CH₂OH and —NH—SO₂—R²,
wherein the 5-membered heteroaryl contains at least two heteroatoms, and
wherein the C₁-C₆ alkyl is optionally substituted with one or more substituents independently selected from 5-membered heteroaryl, wherein the 5-membered heteroaryl contains at least two heteroatoms;
R²¹ is selected from hydrogen and nitrile;
R²² is selected from hydrogen and hydroxy;
each R²³ is independently selected from halogen, C₁-C₆ alkyl, C₁-C₆ alkoxy,
wherein the C₁-C₆ alkyl and C₁-C₆ alkoxy are optionally substituted with one or more substituents independently selected from halogens;
each R²⁴ is independently selected from halogen, C₁-C₆ alkyl, C₁-C₆ alkoxy, 5-membered heteroaryl
wherein the C₁-C₆ alkyl and C₁-C₆ alkoxy are optionally substituted with one or more substituents independently selected from halogens;
each X¹ is independently selected from —NR²— and —CR²R³—; and
each X³ is independently selected from NH and O.

2. The compound of claim 1, wherein the compound of Formula (I) is represented by compound of Formula (Ia) or Formula (Ib): 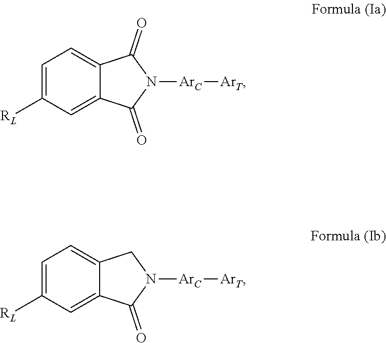
or a pharmaceutically acceptable salt thereof wherein:
Ar_{C} is selected from arylene and heteroarylene; wherein Ar_{C} is substituted with one or more R_{C};
each R_{C} are independently selected from halogen, —CN, optionally substituted C₁-C₆ alkyl, optionally substituted C₁-C₆ alkoxy, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;
wherein the C₁-C₆ alkyl and C₁-C₆ alkoxy are optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl; and
wherein the aryl and heteroaryl are optionally substituted with one or more substituents independently selected from —OH, halogen, —C(═O)OH, —C(═O)NR¹R², C₁-C₆ alkyl, C₁-C₆ alkoxy, and —NR⁷R⁸;
each R¹ and R² is independently selected from hydrogen and C₁-C₆ alkyl optionally substituted with one or more substituents independently selected from halogen, —C(═O)OH, —C(═O)NR⁷R⁸, —OH, aryl, heteroaryl, C₃-C₈ cycloalkyl, and C₂-C₈ heterocycloalkyl;
or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;
each R³ is independently C₁-C₆ alkyl optionally substituted with one or more substituents independently selected from —OH, optionally substituted —OC(═O)C₁-C₆ alkyl, optionally substituted —C(═O)OC₁-C₆ alkyl, C₁-C₆ alkoxy, —C(═O)OH, and —NR¹R²; wherein the —OC(═O)C₁-C₆ alkyl and —C(═O)OC₁-C₆ alkyl are optionally substituted with one or more substituents independently selected from —OH and —NR⁷R⁸;
each R⁴ and R⁵ is independently selected from hydrogen and C₁-C₆ alkyl optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl;
or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;
each R⁶ are independently selected from optionally substituted C₁-C₆ alkyl and optionally substituted aryl;
wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and
wherein the aryl is optionally substituted with one or more substituents independently selected from —OH, halogen, —C(═O)OR⁷, —C(═O)NR⁷R⁸, C₁-C₆ alkyl, C₁-C₆ alkoxy, and —NR⁷R⁸;
each R⁷ and R⁸ is independently selected from hydrogen and C₁-C₆ alkyl;
or R⁷ and R⁸ are taken together with the N to which they are attached to form an optionally substituted C₂-C₈ heterocycloalkyl optionally substituted with one or more C₁-C₆ alkyl substituents;
Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;
R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²; wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR¹R², —C(═O)R¹², aryl, and C₁-C₆ alkyl-(aryl);
R⁹ is C₁-C₆ alkyl optionally substituted with one or more substituent independently selected from —OH and —NR¹R²;
each R¹⁰ and R¹¹ is independently selected from hydrogen and C₁-C₆ alkyl optionally substituted with one or more substituents independently selected from —C(═O)OH, —C(═O)NR¹R², —OH, aryl, hydroxyaryl and heteroaryl;
or R¹⁰ and R¹¹ are taken together with the N to which they are attached to form a C₂-C₈ heterocycloalkyl optionally substituted with one or more C₁-C₆ alkyl substituents;
R¹² is selected from C₁-C₆ alkyl and aryl optionally substituted with one or more C₁-C₆alkyl substituents;
provided that at least one of R_{C} is not —OH when R_{L} is —C(═O)OH in Formula (Ia) or at least one of R_{C} is not —OEt when R_{L} is —C(═O)OH in Formula (Ia).

3. The compound of claim 1, wherein the compound of Formula (I) is selected from the group consisting of: 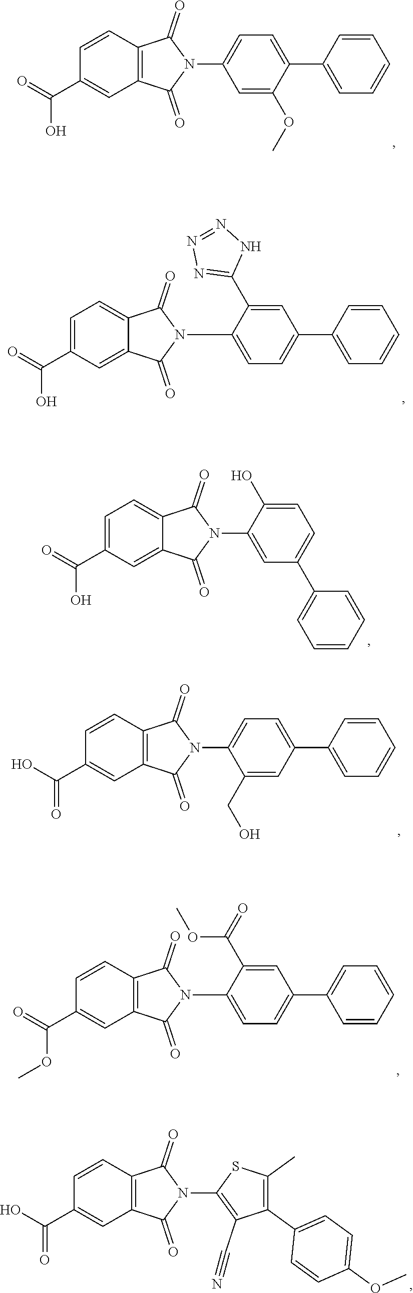 [SMILES: COc1cc(N2C(=O)c3ccc(C(=O)O)cc3C2=O)ccc1-c1ccccc1 ; O=C(O)c1ccc2c(c1)C(=O)N(c1ccc(-c3ccccc3)cc1-c1nnn[nH]1)C2=O ; O=C(O)c1ccc2c(c1)C(=O)N(c1cc(-c3ccccc3)ccc1O)C2=O ; O=C(O)c1ccc2c(c1)C(=O)N(c1ccc(-c3ccccc3)cc1CO)C2=O ; COC(=O)c1ccc2c(c1)C(=O)N(c1ccc(-c3ccccc3)cc1C(=O)OC)C2=O ; COc1ccc(-c2c(C)sc(N3C(=O)c4ccc(C(=O)O)cc4C3=O)c2C#N)cc1] 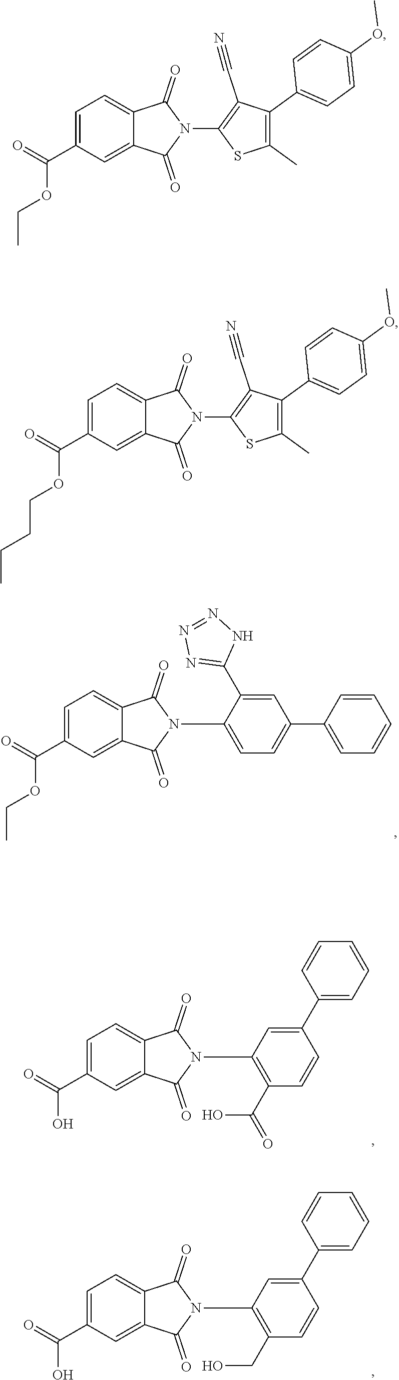 [SMILES: CCOC(=O)c1ccc2c(c1)C(=O)N(c1sc(C)c(-c3ccc(OC)cc3)c1C#N)C2=O ; CCCCOC(=O)c1ccc2c(c1)C(=O)N(c1sc(C)c(-c3ccc(OC)cc3)c1C#N)C2=O ; CCOC(=O)c1ccc2c(c1)C(=O)N(c1ccc(-c3ccccc3)cc1-c1nnn[nH]1)C2=O ; O=C(O)c1ccc2c(c1)C(=O)N(c1cc(-c3ccccc3)ccc1C(=O)O)C2=O ; O=C(O)c1ccc2c(c1)C(=O)N(c1cc(-c3ccccc3)ccc1CO)C2=O] 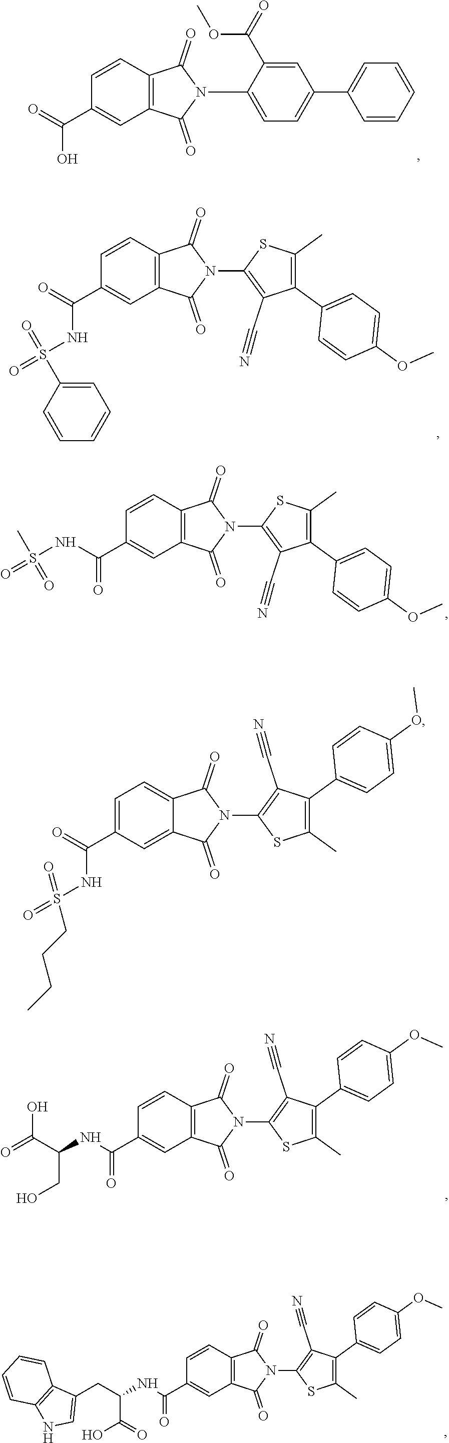 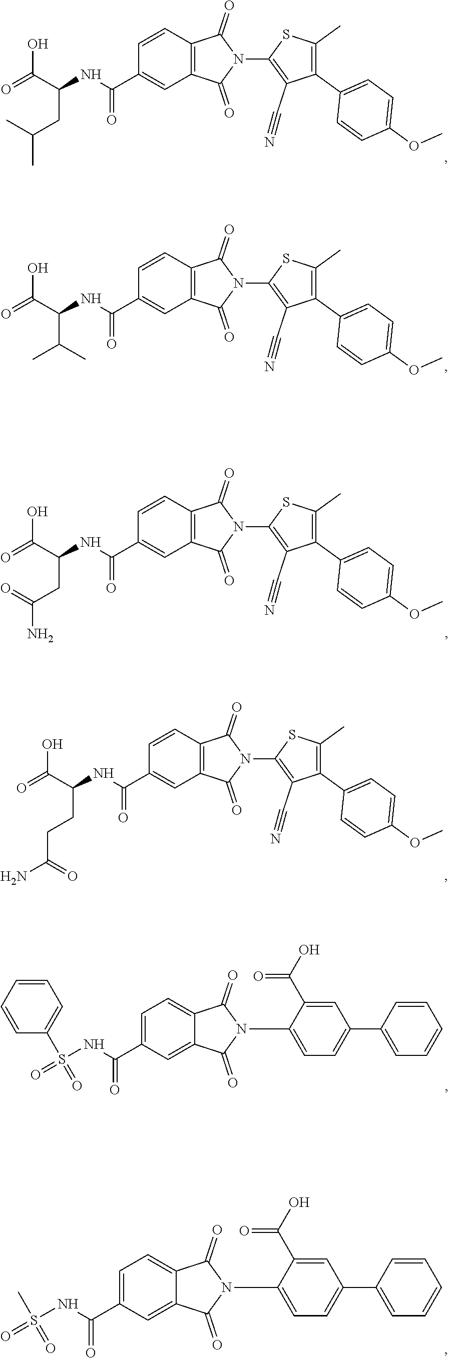 [SMILES: COc1ccc(-c2c(C)sc(N3C(=O)c4ccc(C(=O)N[C@@H](CC(C)C)C(=O)O)cc4C3=O)c2C#N)cc1 ; COc1ccc(-c2c(C)sc(N3C(=O)c4ccc(C(=O)N[C@H](C(=O)O)C(C)C)cc4C3=O)c2C#N)cc1 ; COc1ccc(-c2c(C)sc(N3C(=O)c4ccc(C(=O)N[C@@H](CC(N)=O)C(=O)O)cc4C3=O)c2C#N)cc1 ; COc1ccc(-c2c(C)sc(N3C(=O)c4ccc(C(=O)N[C@@H](CCC(N)=O)C(=O)O)cc4C3=O)c2C#N)cc1 ; O=C(NS(=O)(=O)c1ccccc1)c1ccc2c(c1)C(=O)N(c1ccc(-c3ccccc3)cc1C(=O)O)C2=O ; CS(=O)(=O)NC(=O)c1ccc2c(c1)C(=O)N(c1ccc(-c3ccccc3)cc1C(=O)O)C2=O] 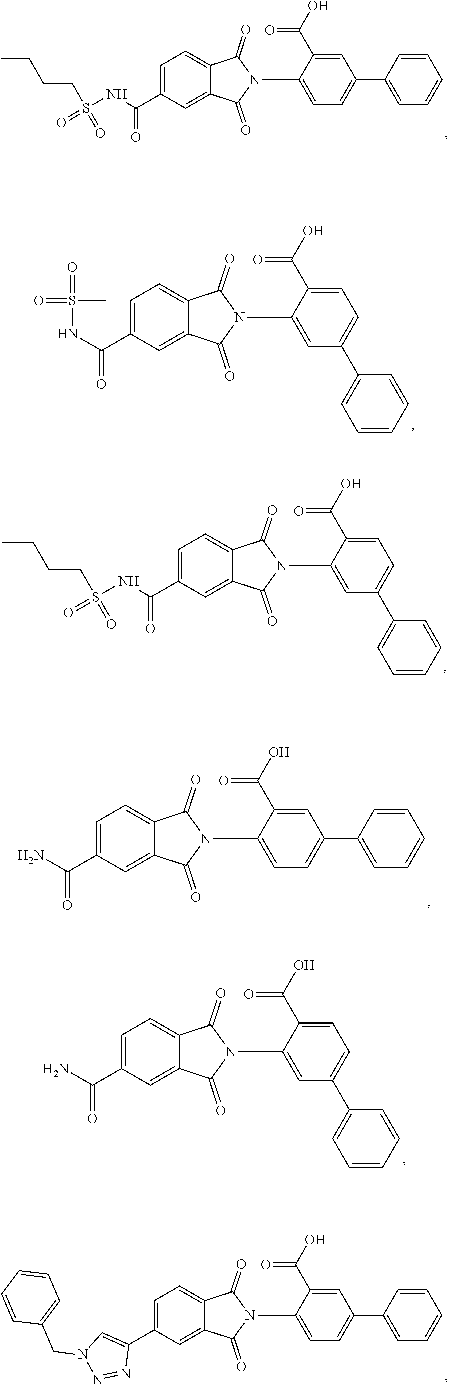 [SMILES: CCCCS(=O)(=O)NC(=O)c1ccc2c(c1)C(=O)N(c1ccc(-c3ccccc3)cc1C(=O)O)C2=O ; CS(=O)(=O)NC(=O)c1ccc2c(c1)C(=O)N(c1cc(-c3ccccc3)ccc1C(=O)O)C2=O ; CCCCS(=O)(=O)NC(=O)c1ccc2c(c1)C(=O)N(c1cc(-c3ccccc3)ccc1C(=O)O)C2=O ; NC(=O)c1ccc2c(c1)C(=O)N(c1ccc(-c3ccccc3)cc1C(=O)O)C2=O ; NC(=O)c1ccc2c(c1)C(=O)N(c1cc(-c3ccccc3)ccc1C(=O)O)C2=O ; O=C(O)c1cc(-c2ccccc2)ccc1N1C(=O)c2ccc(-c3cn(Cc4ccccc4)nn3)cc2C1=O] 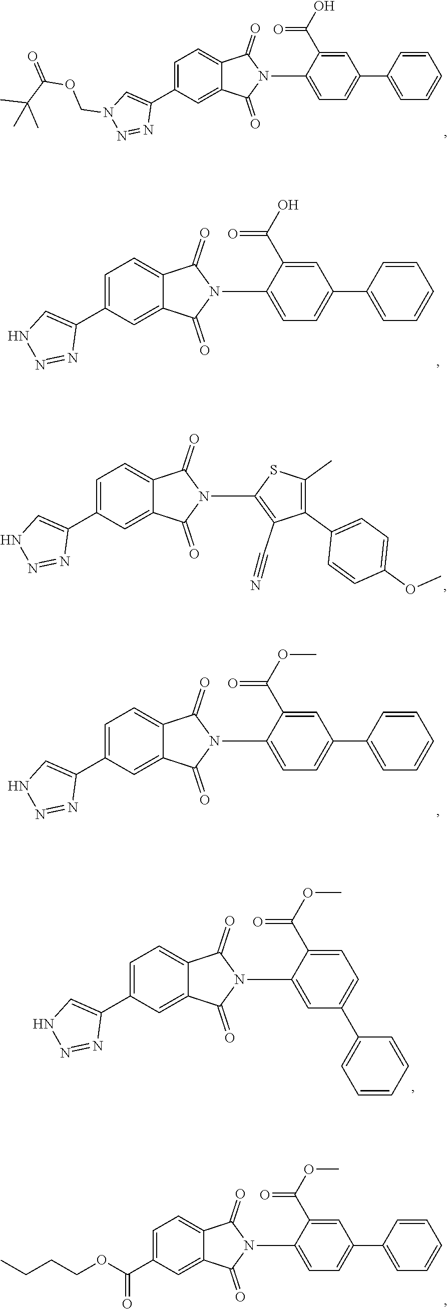 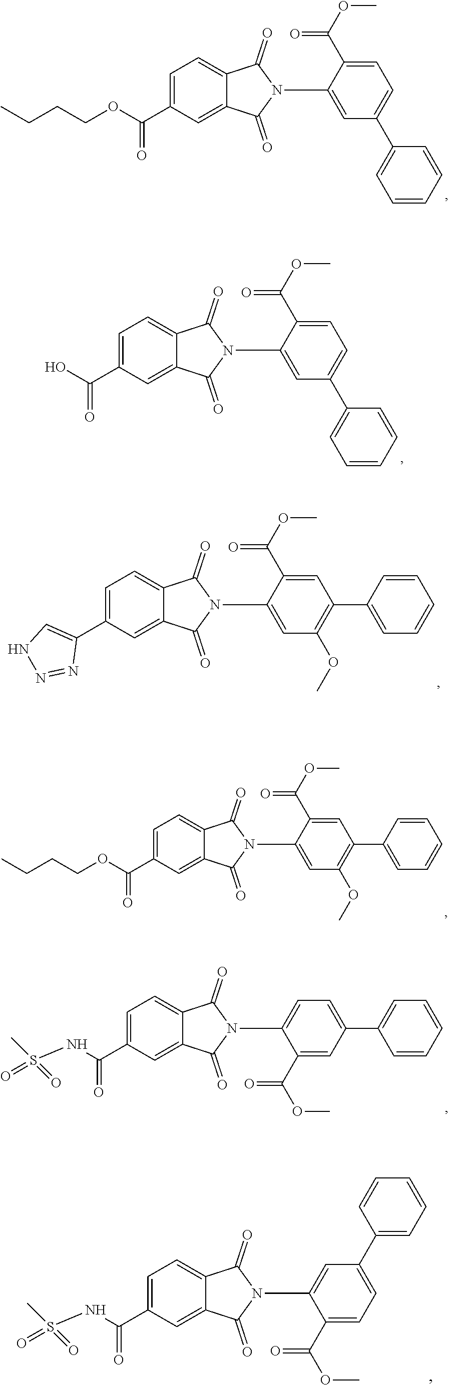 [SMILES: CCCCOC(=O)c1ccc2c(c1)C(=O)N(c1cc(-c3ccccc3)ccc1C(=O)OC)C2=O ; COC(=O)c1ccc(-c2ccccc2)cc1N1C(=O)c2ccc(C(=O)O)cc2C1=O ; COC(=O)c1cc(-c2ccccc2)c(OC)cc1N1C(=O)c2ccc(-c3c[nH]nn3)cc2C1=O ; CCCCOC(=O)c1ccc2c(c1)C(=O)N(c1cc(OC)c(-c3ccccc3)cc1C(=O)OC)C2=O ; COC(=O)c1cc(-c2ccccc2)ccc1N1C(=O)c2ccc(C(=O)NS(C)(=O)=O)cc2C1=O ; COC(=O)c1ccc(-c2ccccc2)cc1N1C(=O)c2ccc(C(=O)NS(C)(=O)=O)cc2C1=O] 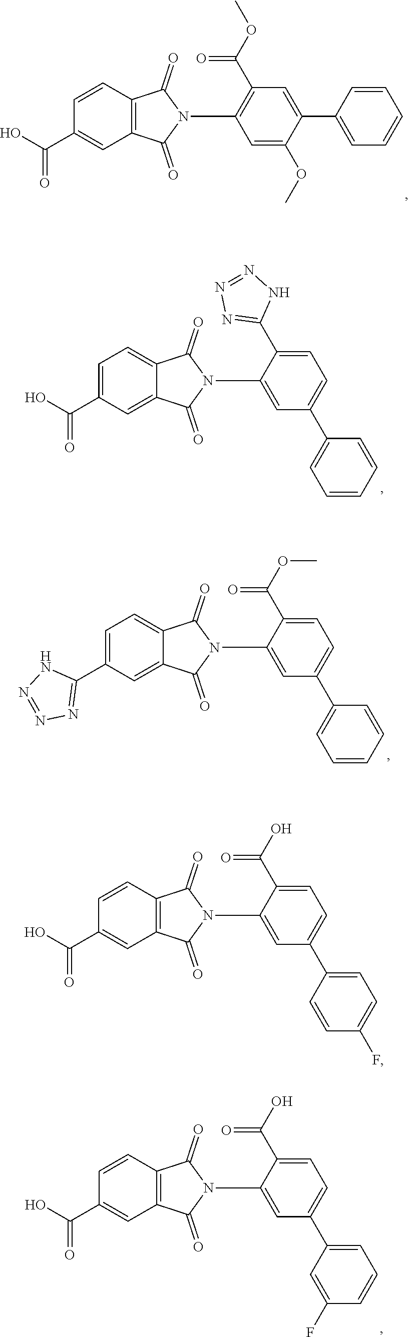 [SMILES: COC(=O)c1cc(-c2ccccc2)c(OC)cc1N1C(=O)c2ccc(C(=O)O)cc2C1=O ; O=C(O)c1ccc2c(c1)C(=O)N(c1cc(-c3ccccc3)ccc1-c1nnn[nH]1)C2=O ; COC(=O)c1ccc(-c2ccccc2)cc1N1C(=O)c2ccc(-c3nnn[nH]3)cc2C1=O ; O=C(O)c1ccc2c(c1)C(=O)N(c1cc(-c3ccc(F)cc3)ccc1C(=O)O)C2=O ; O=C(O)c1ccc2c(c1)C(=O)N(c1cc(-c3cccc(F)c3)ccc1C(=O)O)C2=O] 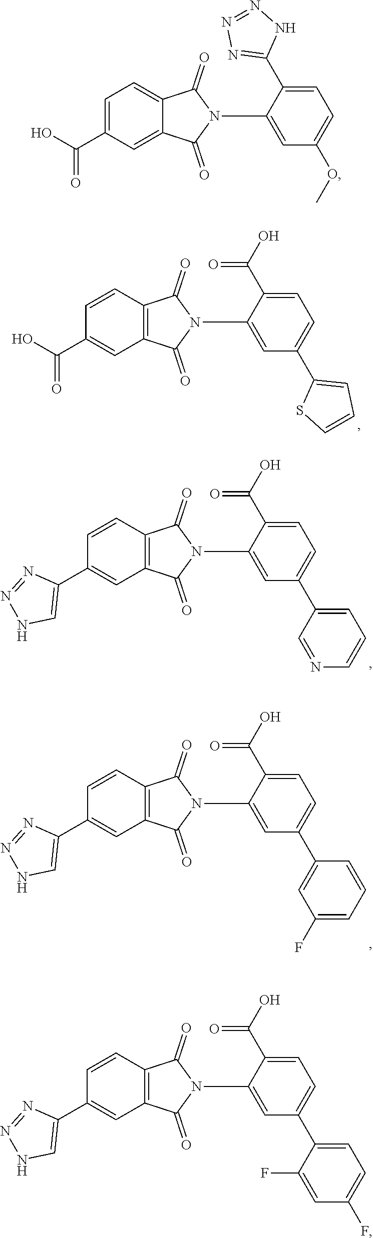 [SMILES: COc1ccc(-c2nnn[nH]2)c(N2C(=O)c3ccc(C(=O)O)cc3C2=O)c1 ; O=C(O)c1ccc2c(c1)C(=O)N(c1cc(-c3cccs3)ccc1C(=O)O)C2=O ; O=C(O)c1ccc(-c2cccnc2)cc1N1C(=O)c2ccc(-c3c[nH]nn3)cc2C1=O ; O=C(O)c1ccc(-c2cccc(F)c2)cc1N1C(=O)c2ccc(-c3c[nH]nn3)cc2C1=O ; O=C(O)c1ccc(-c2ccc(F)cc2F)cc1N1C(=O)c2ccc(-c3c[nH]nn3)cc2C1=O] 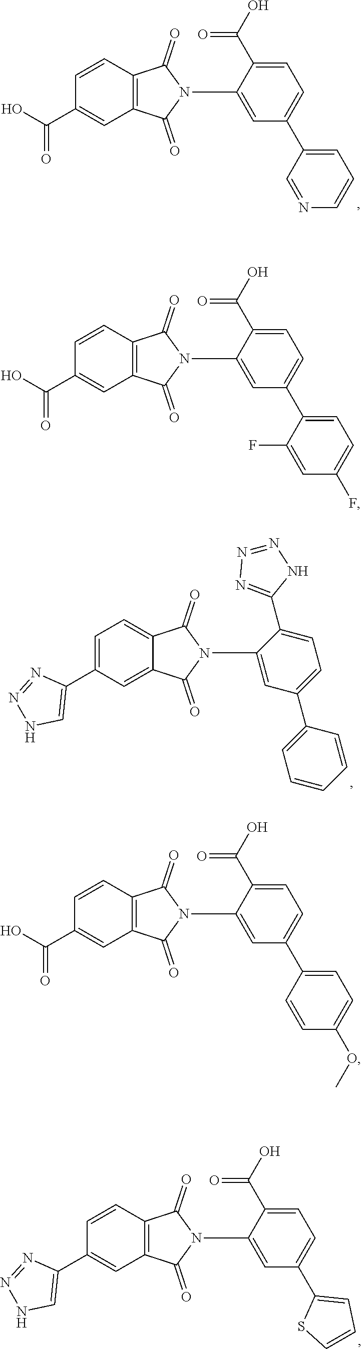 [SMILES: O=C(O)c1ccc2c(c1)C(=O)N(c1cc(-c3cccnc3)ccc1C(=O)O)C2=O ; O=C(O)c1ccc2c(c1)C(=O)N(c1cc(-c3ccc(F)cc3F)ccc1C(=O)O)C2=O ; O=C1c2ccc(-c3c[nH]nn3)cc2C(=O)N1c1cc(-c2ccccc2)ccc1-c1nnn[nH]1 ; COc1ccc(-c2ccc(C(=O)O)c(N3C(=O)c4ccc(C(=O)O)cc4C3=O)c2)cc1 ; O=C(O)c1ccc(-c2cccs2)cc1N1C(=O)c2ccc(-c3c[nH]nn3)cc2C1=O] 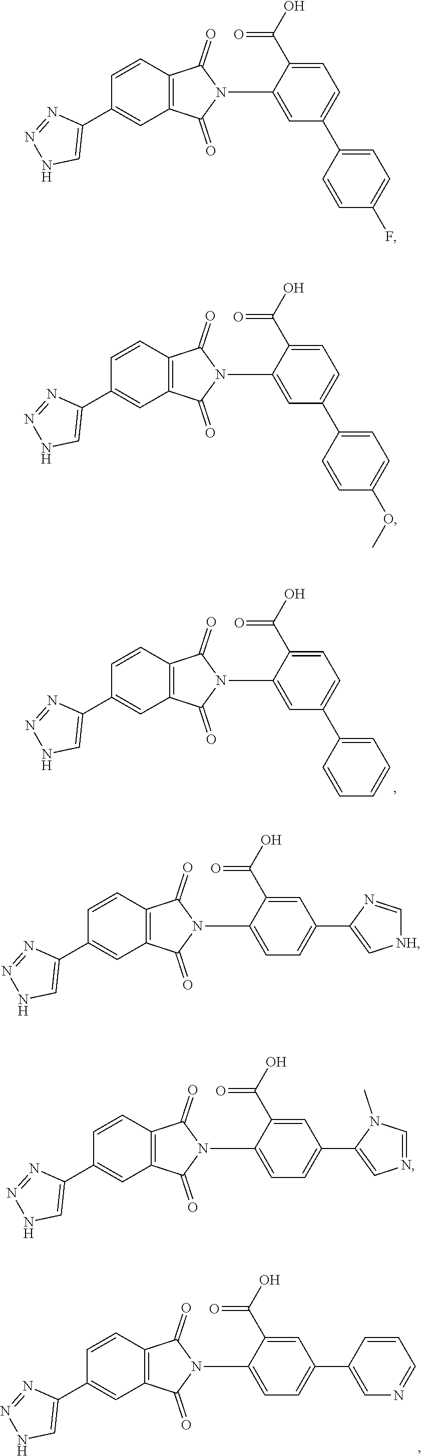 [SMILES: O=C(O)c1ccc(-c2ccc(F)cc2)cc1N1C(=O)c2ccc(-c3c[nH]nn3)cc2C1=O ; COc1ccc(-c2ccc(C(=O)O)c(N3C(=O)c4ccc(-c5c[nH]nn5)cc4C3=O)c2)cc1 ; O=C(O)c1ccc(-c2ccccc2)cc1N1C(=O)c2ccc(-c3c[nH]nn3)cc2C1=O ; O=C(O)c1cc(-c2c[nH]cn2)ccc1N1C(=O)c2ccc(-c3c[nH]nn3)cc2C1=O ; Cn1cncc1-c1ccc(N2C(=O)c3ccc(-c4c[nH]nn4)cc3C2=O)c(C(=O)O)c1 ; O=C(O)c1cc(-c2cccnc2)ccc1N1C(=O)c2ccc(-c3c[nH]nn3)cc2C1=O] 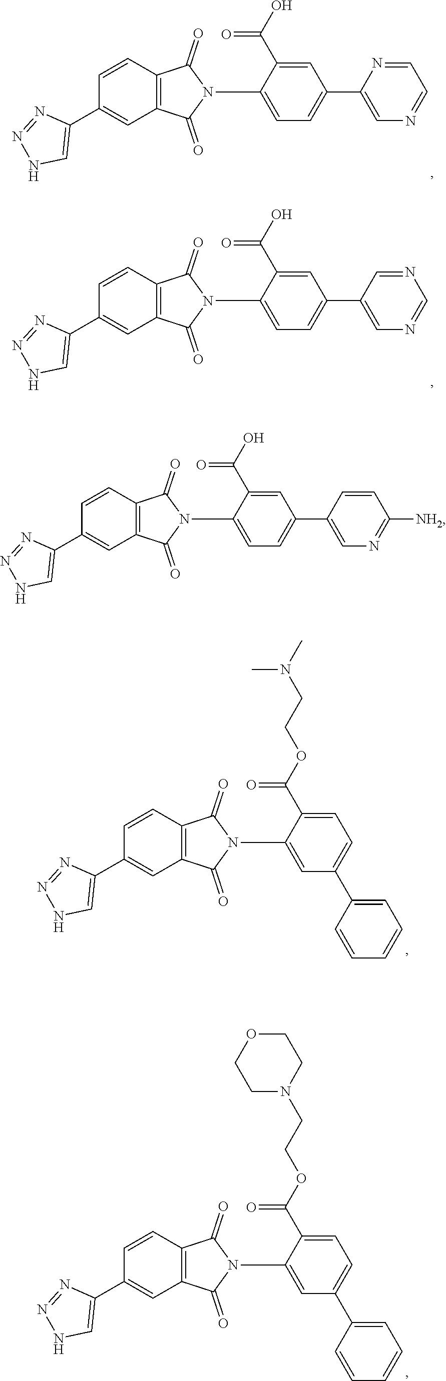 [SMILES: O=C(O)c1cc(-c2cnccn2)ccc1N1C(=O)c2ccc(-c3c[nH]nn3)cc2C1=O ; O=C(O)c1cc(-c2cncnc2)ccc1N1C(=O)c2ccc(-c3c[nH]nn3)cc2C1=O ; Nc1ccc(-c2ccc(N3C(=O)c4ccc(-c5c[nH]nn5)cc4C3=O)c(C(=O)O)c2)cn1 ; CN(C)CCOC(=O)c1ccc(-c2ccccc2)cc1N1C(=O)c2ccc(-c3c[nH]nn3)cc2C1=O ; O=C(OCCN1CCOCC1)c1ccc(-c2ccccc2)cc1N1C(=O)c2ccc(-c3c[nH]nn3)cc2C1=O] 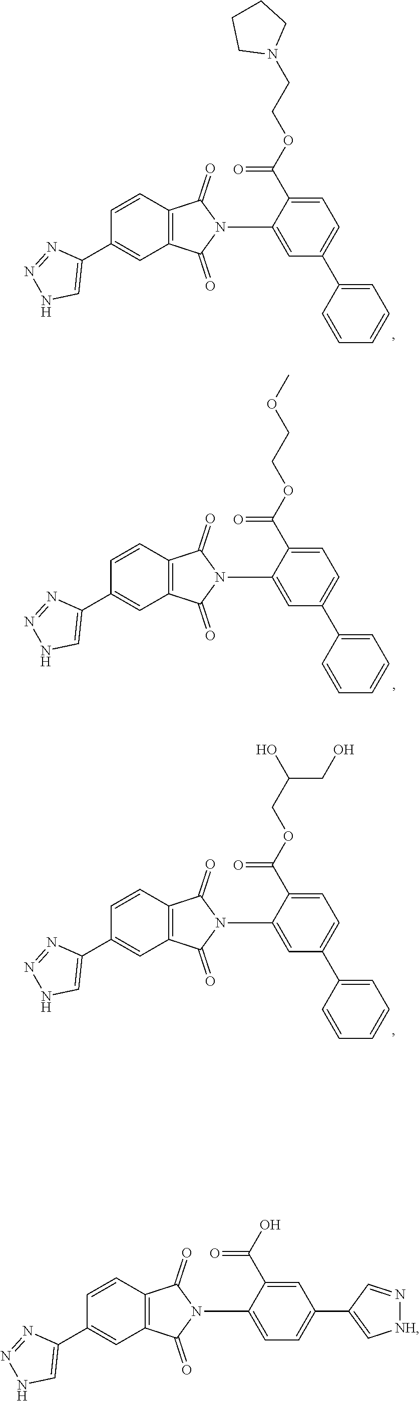 [SMILES: O=C(OCCN1CCCC1)c1ccc(-c2ccccc2)cc1N1C(=O)c2ccc(-c3c[nH]nn3)cc2C1=O ; COCCOC(=O)c1ccc(-c2ccccc2)cc1N1C(=O)c2ccc(-c3c[nH]nn3)cc2C1=O ; O=C(OCC(O)CO)c1ccc(-c2ccccc2)cc1N1C(=O)c2ccc(-c3c[nH]nn3)cc2C1=O ; O=C(O)c1cc(-c2cn[nH]c2)ccc1N1C(=O)c2ccc(-c3c[nH]nn3)cc2C1=O] 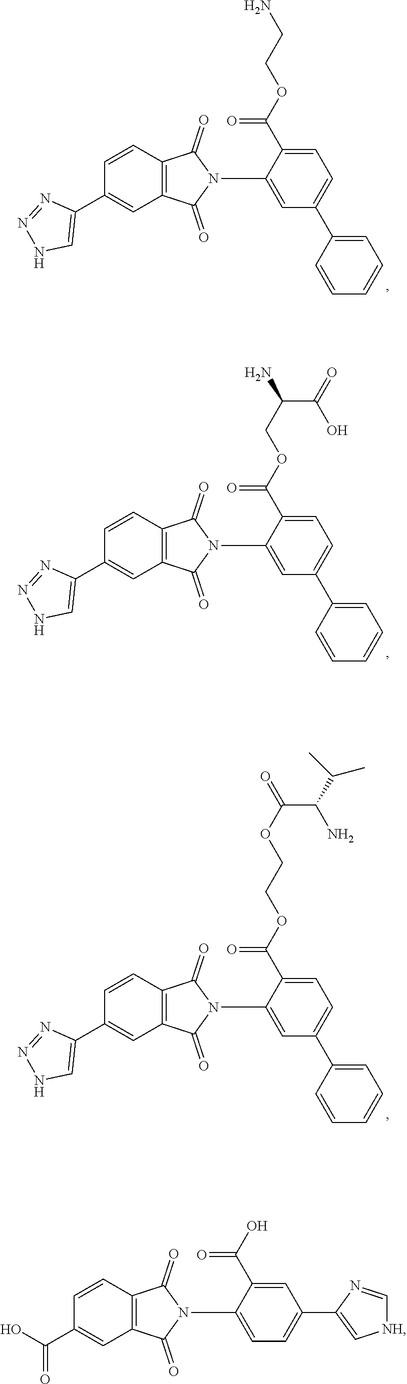 [SMILES: NCCOC(=O)c1ccc(-c2ccccc2)cc1N1C(=O)c2ccc(-c3c[nH]nn3)cc2C1=O ; N[C@H](COC(=O)c1ccc(-c2ccccc2)cc1N1C(=O)c2ccc(-c3c[nH]nn3)cc2C1=O)C(=O)O ; CC(C)[C@H](N)C(=O)OCCOC(=O)c1ccc(-c2ccccc2)cc1N1C(=O)c2ccc(-c3c[nH]nn3)cc2C1=O ; O=C(O)c1ccc2c(c1)C(=O)N(c1ccc(-c3c[nH]cn3)cc1C(=O)O)C2=O] 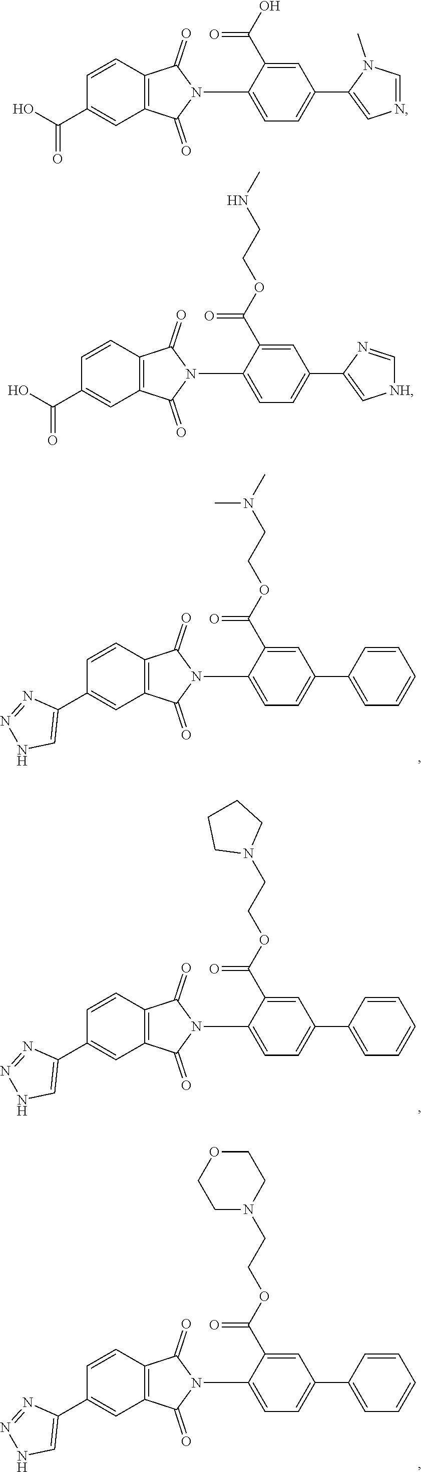 [SMILES: Cn1cncc1-c1ccc(N2C(=O)c3ccc(C(=O)O)cc3C2=O)c(C(=O)O)c1 ; CNCCOC(=O)c1cc(-c2c[nH]cn2)ccc1N1C(=O)c2ccc(C(=O)O)cc2C1=O ; CN(C)CCOC(=O)c1cc(-c2ccccc2)ccc1N1C(=O)c2ccc(-c3c[nH]nn3)cc2C1=O ; O=C(OCCN1CCCC1)c1cc(-c2ccccc2)ccc1N1C(=O)c2ccc(-c3c[nH]nn3)cc2C1=O ; O=C(OCCN1CCOCC1)c1cc(-c2ccccc2)ccc1N1C(=O)c2ccc(-c3c[nH]nn3)cc2C1=O] 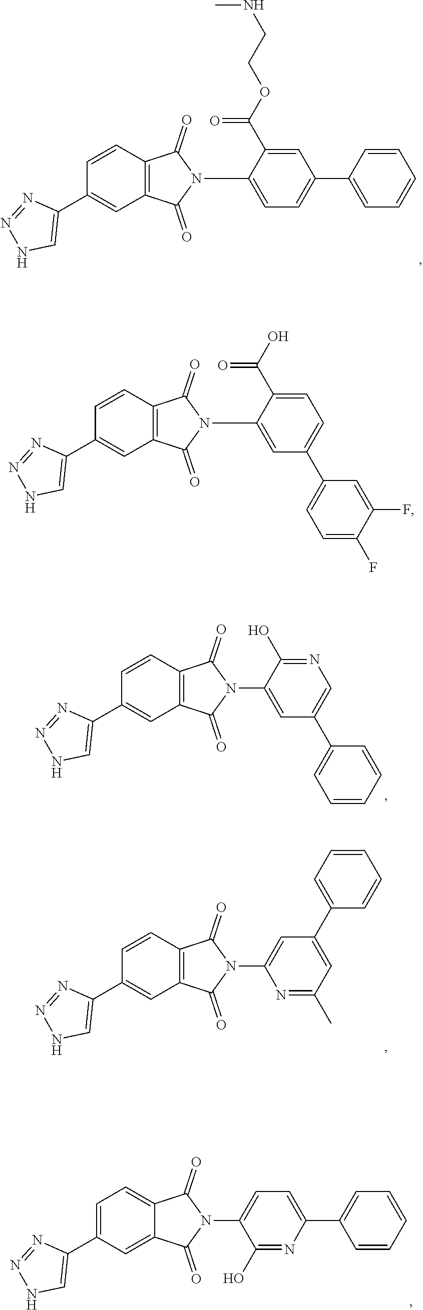 [SMILES: CNCCOC(=O)c1cc(-c2ccccc2)ccc1N1C(=O)c2ccc(-c3c[nH]nn3)cc2C1=O ; O=C(O)c1ccc(-c2ccc(F)c(F)c2)cc1N1C(=O)c2ccc(-c3c[nH]nn3)cc2C1=O ; O=C1c2ccc(-c3c[nH]nn3)cc2C(=O)N1c1cc(-c2ccccc2)cnc1O ; Cc1cc(-c2ccccc2)cc(N2C(=O)c3ccc(-c4c[nH]nn4)cc3C2=O)n1 ; O=C1c2ccc(-c3c[nH]nn3)cc2C(=O)N1c1ccc(-c2ccccc2)nc1O] 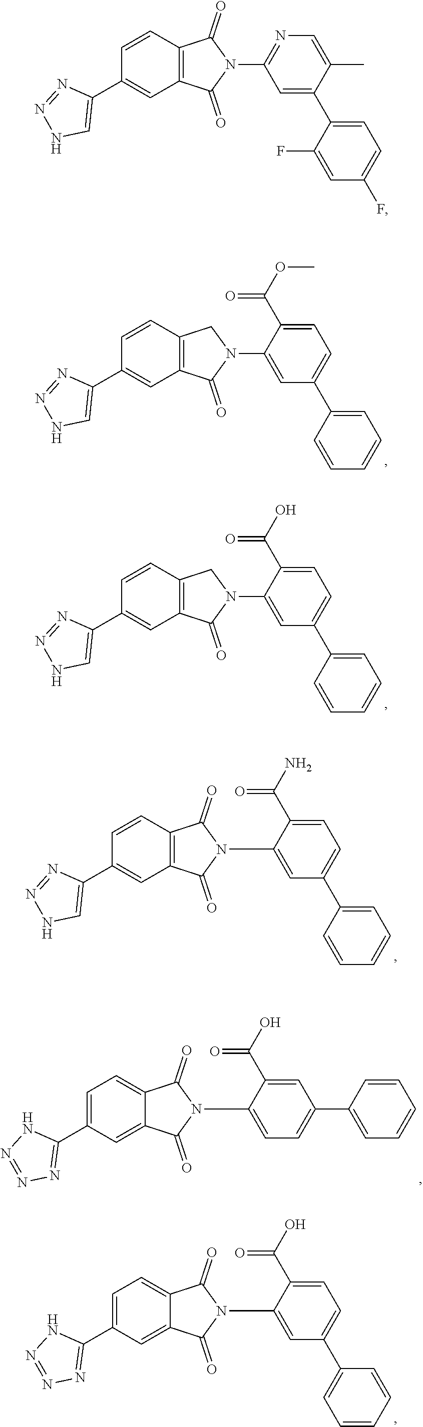 [SMILES: Cc1cnc(N2C(=O)c3ccc(-c4c[nH]nn4)cc3C2=O)cc1-c1ccc(F)cc1F ; COC(=O)c1ccc(-c2ccccc2)cc1N1Cc2ccc(-c3c[nH]nn3)cc2C1=O ; O=C(O)c1ccc(-c2ccccc2)cc1N1Cc2ccc(-c3c[nH]nn3)cc2C1=O ; NC(=O)c1ccc(-c2ccccc2)cc1N1C(=O)c2ccc(-c3c[nH]nn3)cc2C1=O ; O=C(O)c1cc(-c2ccccc2)ccc1N1C(=O)c2ccc(-c3nnn[nH]3)cc2C1=O ; O=C(O)c1ccc(-c2ccccc2)cc1N1C(=O)c2ccc(-c3nnn[nH]3)cc2C1=O] 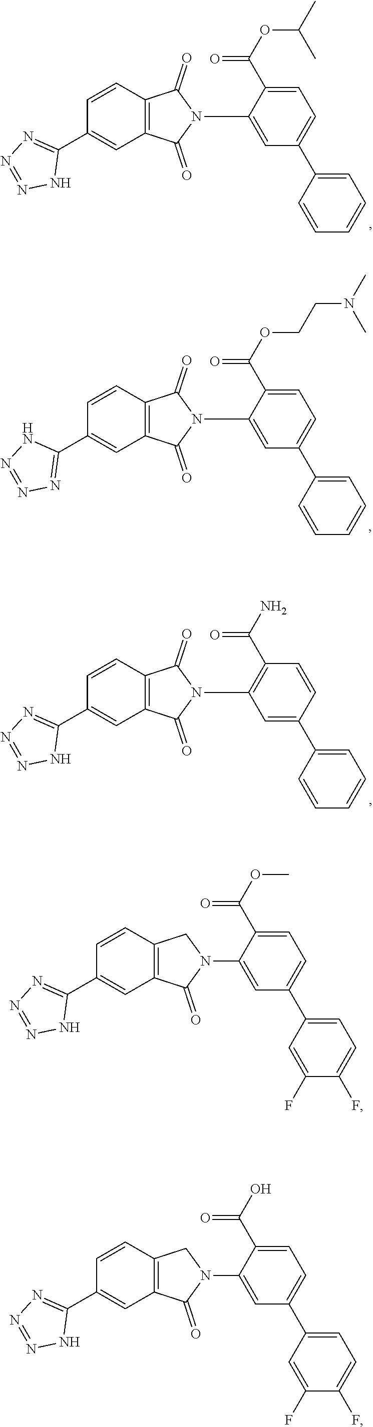 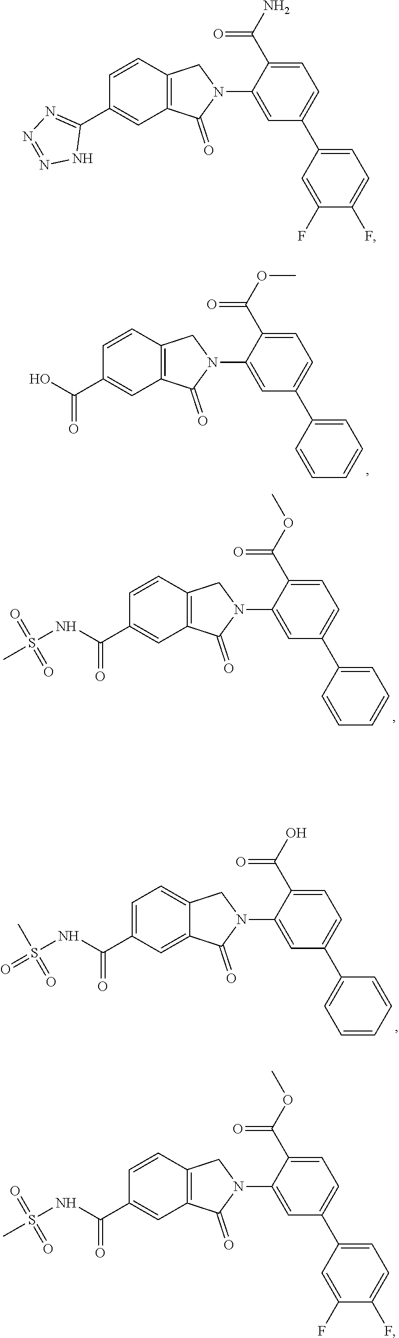 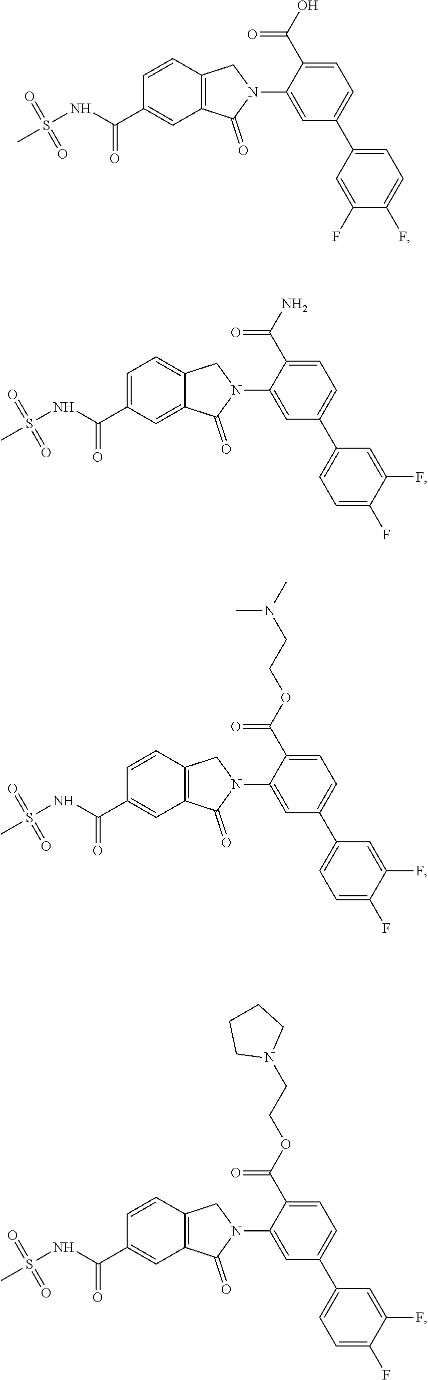 [SMILES: CS(=O)(=O)NC(=O)c1ccc2c(c1)C(=O)N(c1cc(-c3ccc(F)c(F)c3)ccc1C(=O)O)C2 ; CS(=O)(=O)NC(=O)c1ccc2c(c1)C(=O)N(c1cc(-c3ccc(F)c(F)c3)ccc1C(N)=O)C2 ; CN(C)CCOC(=O)c1ccc(-c2ccc(F)c(F)c2)cc1N1Cc2ccc(C(=O)NS(C)(=O)=O)cc2C1=O ; CS(=O)(=O)NC(=O)c1ccc2c(c1)C(=O)N(c1cc(-c3ccc(F)c(F)c3)ccc1C(=O)OCCN1CCCC1)C2] 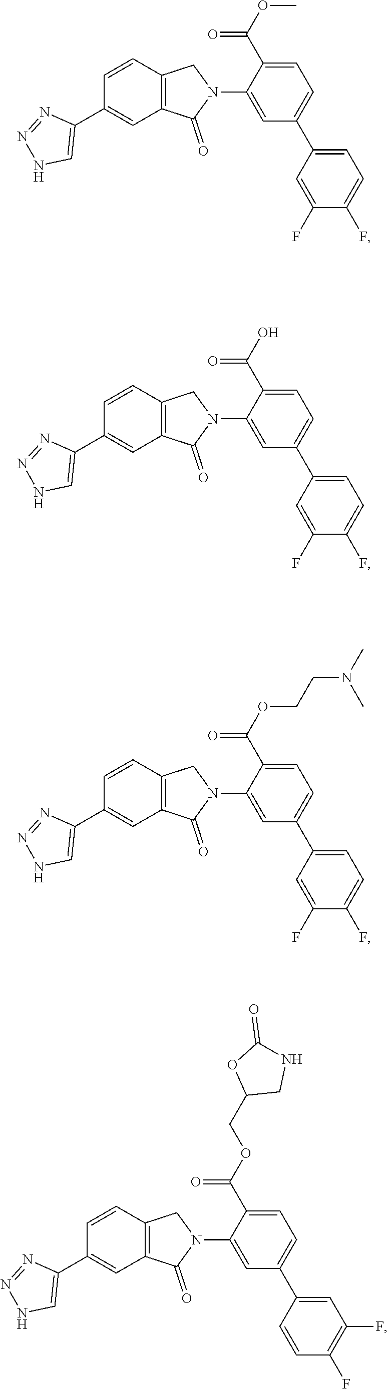 [SMILES: COC(=O)c1ccc(-c2ccc(F)c(F)c2)cc1N1Cc2ccc(-c3c[nH]nn3)cc2C1=O ; O=C(O)c1ccc(-c2ccc(F)c(F)c2)cc1N1Cc2ccc(-c3c[nH]nn3)cc2C1=O ; CN(C)CCOC(=O)c1ccc(-c2ccc(F)c(F)c2)cc1N1Cc2ccc(-c3c[nH]nn3)cc2C1=O ; O=C1NCC(COC(=O)c2ccc(-c3ccc(F)c(F)c3)cc2N2Cc3ccc(-c4c[nH]nn4)cc3C2=O)O1] 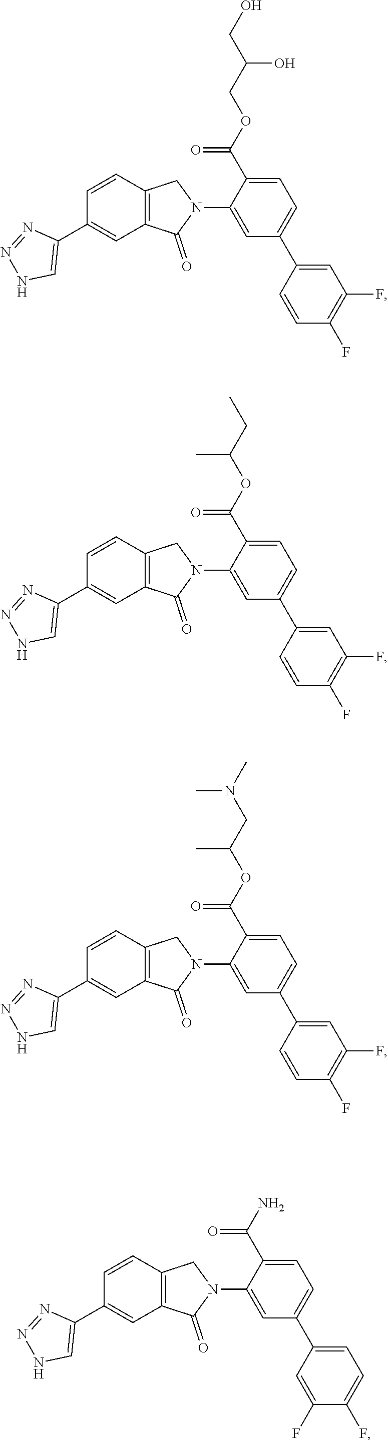 [SMILES: O=C(OCC(O)CO)c1ccc(-c2ccc(F)c(F)c2)cc1N1Cc2ccc(-c3c[nH]nn3)cc2C1=O ; CCC(C)OC(=O)c1ccc(-c2ccc(F)c(F)c2)cc1N1Cc2ccc(-c3c[nH]nn3)cc2C1=O ; CC(CN(C)C)OC(=O)c1ccc(-c2ccc(F)c(F)c2)cc1N1Cc2ccc(-c3c[nH]nn3)cc2C1=O ; NC(=O)c1ccc(-c2ccc(F)c(F)c2)cc1N1Cc2ccc(-c3c[nH]nn3)cc2C1=O] 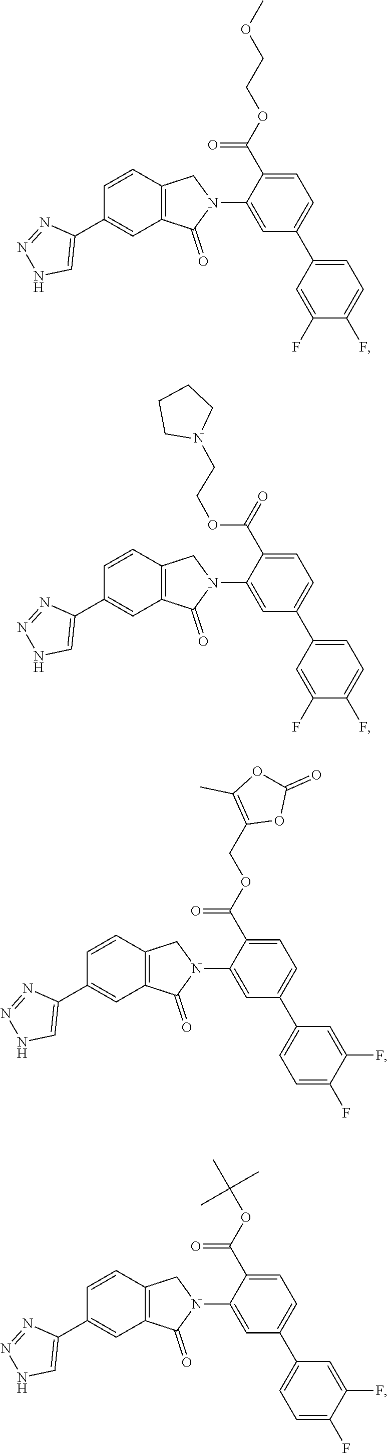 [SMILES: COCCOC(=O)c1ccc(-c2ccc(F)c(F)c2)cc1N1Cc2ccc(-c3c[nH]nn3)cc2C1=O ; O=C(OCCN1CCCC1)c1ccc(-c2ccc(F)c(F)c2)cc1N1Cc2ccc(-c3c[nH]nn3)cc2C1=O ; Cc1oc(=O)oc1COC(=O)c1ccc(-c2ccc(F)c(F)c2)cc1N1Cc2ccc(-c3c[nH]nn3)cc2C1=O ; CC(C)(C)OC(=O)c1ccc(-c2ccc(F)c(F)c2)cc1N1Cc2ccc(-c3c[nH]nn3)cc2C1=O] 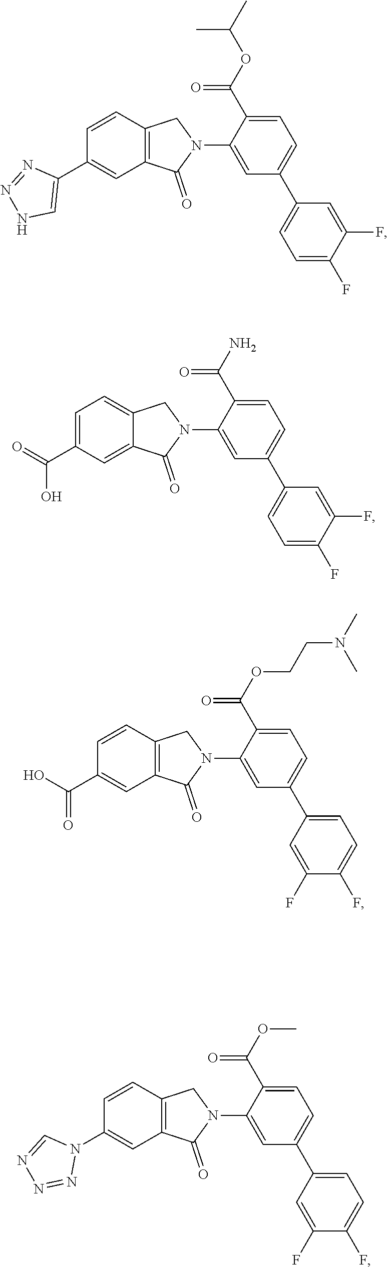 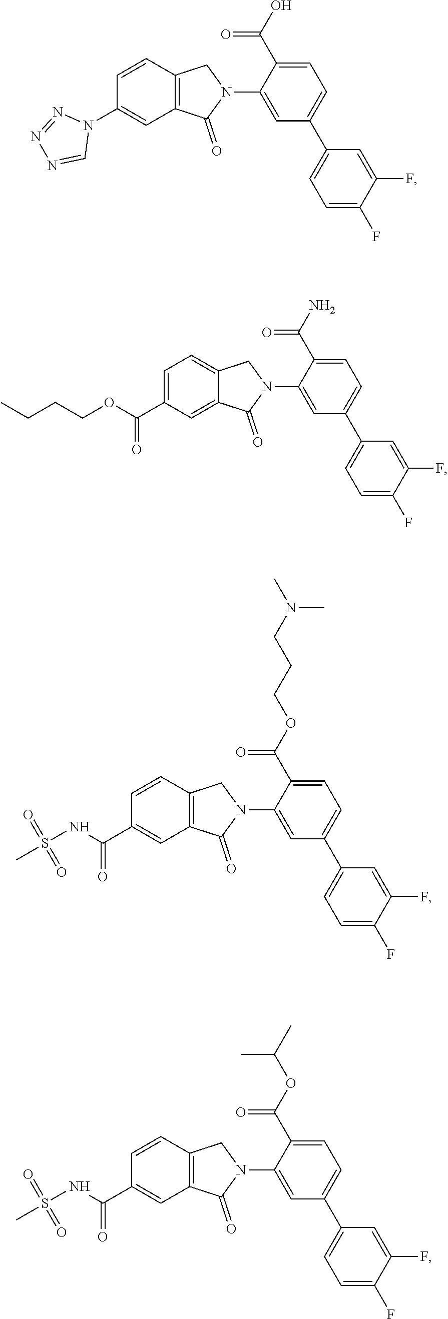 [SMILES: O=C(O)c1ccc(-c2ccc(F)c(F)c2)cc1N1Cc2ccc(-n3cnnn3)cc2C1=O ; CCCCOC(=O)c1ccc2c(c1)C(=O)N(c1cc(-c3ccc(F)c(F)c3)ccc1C(N)=O)C2 ; CN(C)CCCOC(=O)c1ccc(-c2ccc(F)c(F)c2)cc1N1Cc2ccc(C(=O)NS(C)(=O)=O)cc2C1=O ; CC(C)OC(=O)c1ccc(-c2ccc(F)c(F)c2)cc1N1Cc2ccc(C(=O)NS(C)(=O)=O)cc2C1=O] 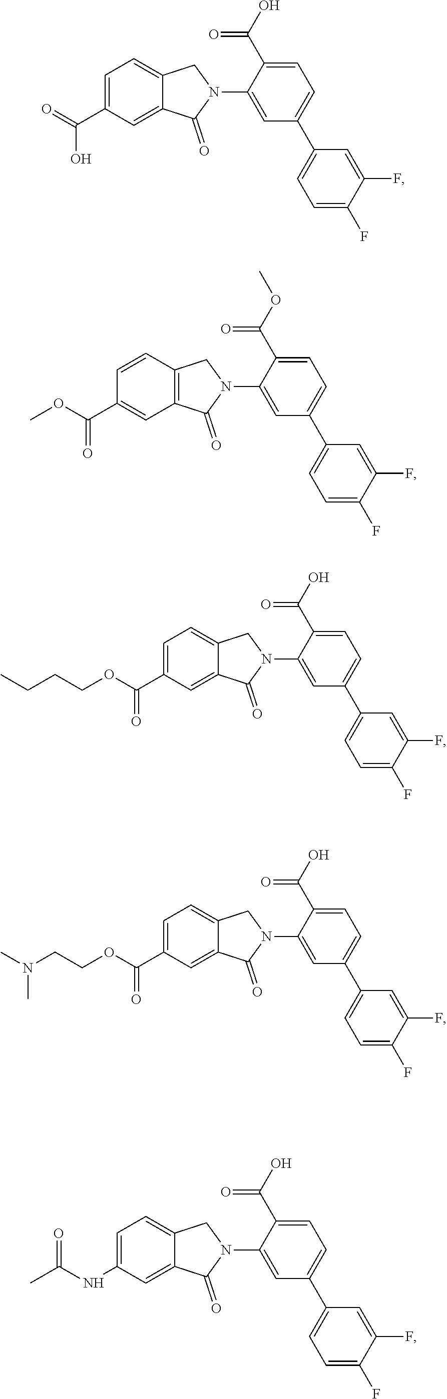 [SMILES: O=C(O)c1ccc2c(c1)C(=O)N(c1cc(-c3ccc(F)c(F)c3)ccc1C(=O)O)C2 ; COC(=O)c1ccc2c(c1)C(=O)N(c1cc(-c3ccc(F)c(F)c3)ccc1C(=O)OC)C2 ; CCCCOC(=O)c1ccc2c(c1)C(=O)N(c1cc(-c3ccc(F)c(F)c3)ccc1C(=O)O)C2 ; CN(C)CCOC(=O)c1ccc2c(c1)C(=O)N(c1cc(-c3ccc(F)c(F)c3)ccc1C(=O)O)C2 ; CC(=O)Nc1ccc2c(c1)C(=O)N(c1cc(-c3ccc(F)c(F)c3)ccc1C(=O)O)C2] 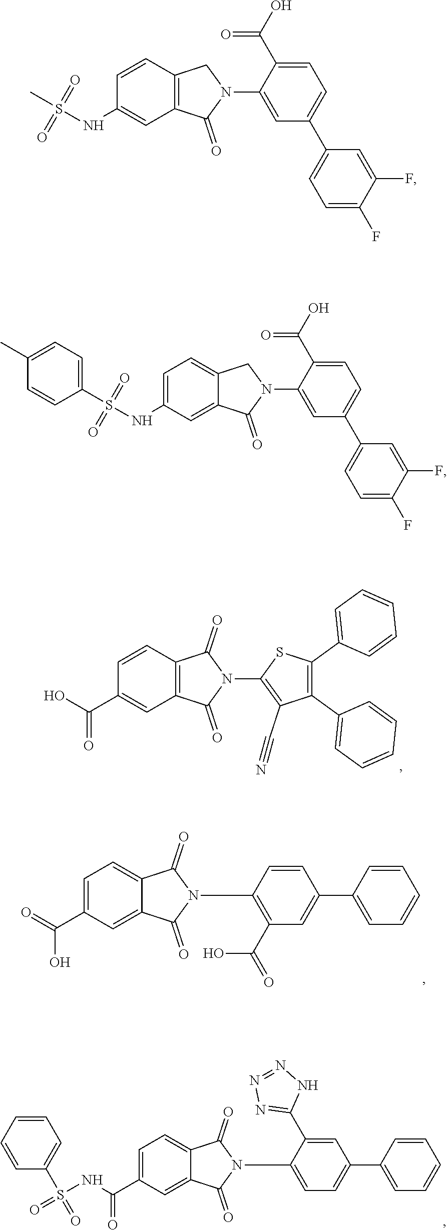 [SMILES: CS(=O)(=O)Nc1ccc2c(c1)C(=O)N(c1cc(-c3ccc(F)c(F)c3)ccc1C(=O)O)C2 ; Cc1ccc(S(=O)(=O)Nc2ccc3c(c2)C(=O)N(c2cc(-c4ccc(F)c(F)c4)ccc2C(=O)O)C3)cc1 ; N#Cc1c(N2C(=O)c3ccc(C(=O)O)cc3C2=O)sc(-c2ccccc2)c1-c1ccccc1 ; O=C(O)c1ccc2c(c1)C(=O)N(c1ccc(-c3ccccc3)cc1C(=O)O)C2=O ; O=C(NS(=O)(=O)c1ccccc1)c1ccc2c(c1)C(=O)N(c1ccc(-c3ccccc3)cc1-c1nnn[nH]1)C2=O] 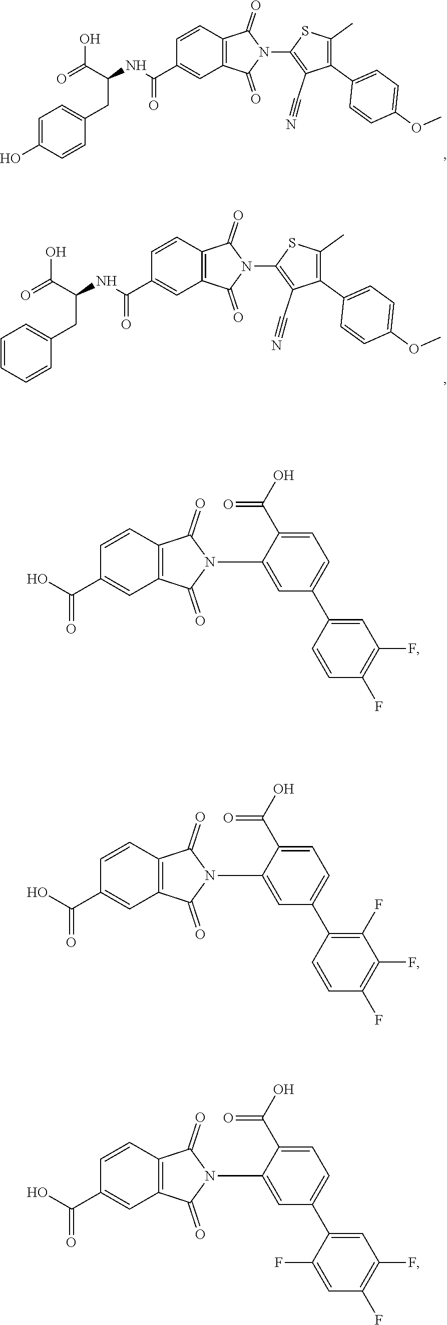 [SMILES: COc1ccc(-c2c(C)sc(N3C(=O)c4ccc(C(=O)N[C@@H](Cc5ccc(O)cc5)C(=O)O)cc4C3=O)c2C#N)cc1 ; COc1ccc(-c2c(C)sc(N3C(=O)c4ccc(C(=O)N[C@@H](Cc5ccccc5)C(=O)O)cc4C3=O)c2C#N)cc1 ; O=C(O)c1ccc2c(c1)C(=O)N(c1cc(-c3ccc(F)c(F)c3)ccc1C(=O)O)C2=O ; O=C(O)c1ccc2c(c1)C(=O)N(c1cc(-c3ccc(F)c(F)c3F)ccc1C(=O)O)C2=O ; O=C(O)c1ccc2c(c1)C(=O)N(c1cc(-c3cc(F)c(F)cc3F)ccc1C(=O)O)C2=O] 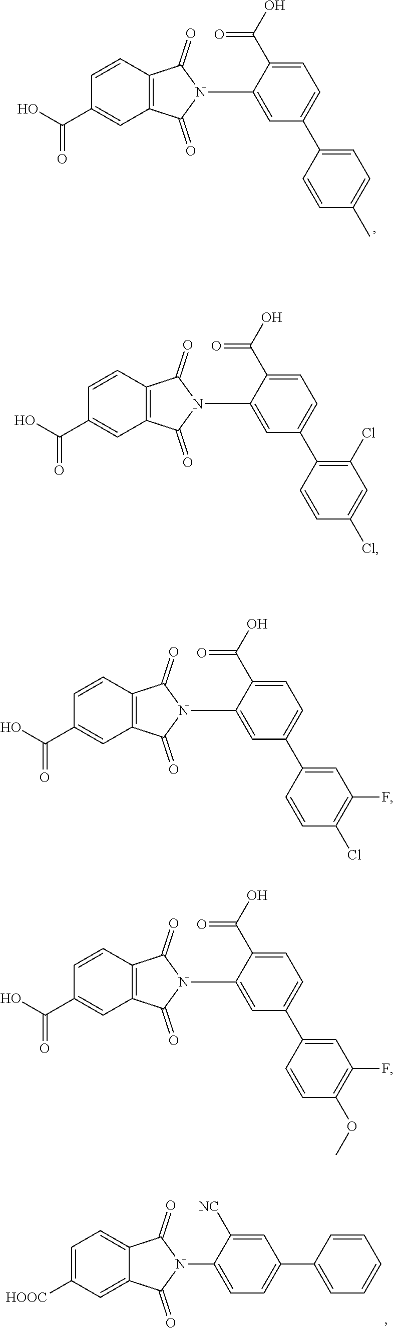 [SMILES: Cc1ccc(-c2ccc(C(=O)O)c(N3C(=O)c4ccc(C(=O)O)cc4C3=O)c2)cc1 ; O=C(O)c1ccc2c(c1)C(=O)N(c1cc(-c3ccc(Cl)cc3Cl)ccc1C(=O)O)C2=O ; O=C(O)c1ccc2c(c1)C(=O)N(c1cc(-c3ccc(Cl)c(F)c3)ccc1C(=O)O)C2=O ; COc1ccc(-c2ccc(C(=O)O)c(N3C(=O)c4ccc(C(=O)O)cc4C3=O)c2)cc1F ; N#Cc1cc(-c2ccccc2)ccc1N1C(=O)c2ccc(C(=O)O)cc2C1=O] 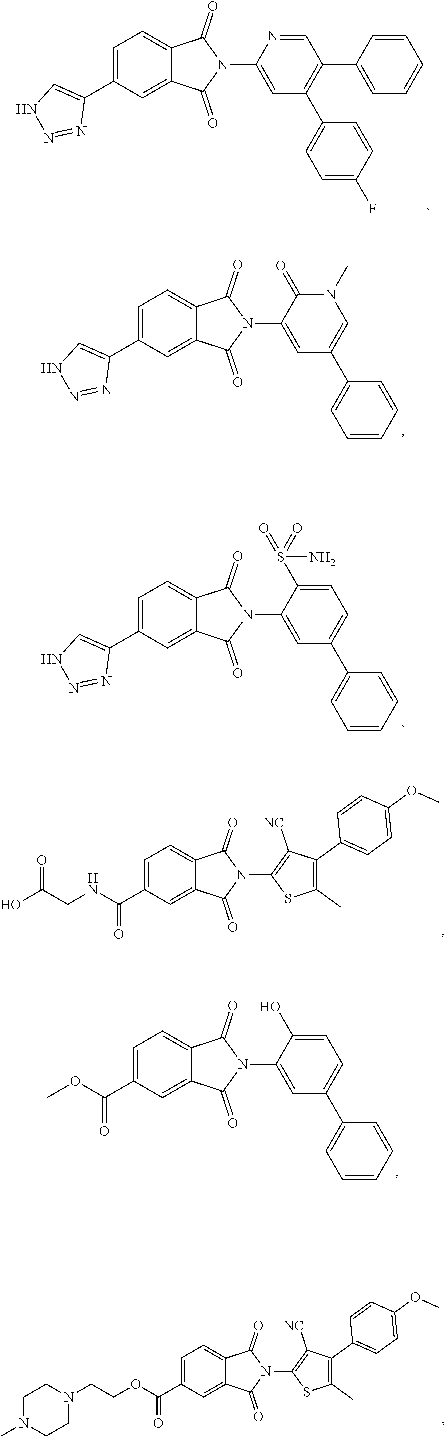 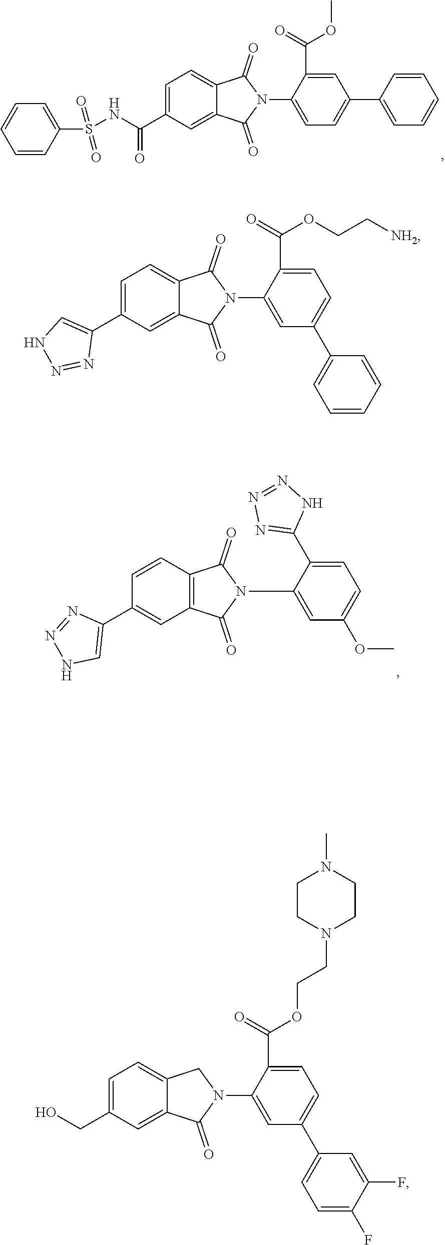 [SMILES: COC(=O)c1cc(-c2ccccc2)ccc1N1C(=O)c2ccc(C(=O)NS(=O)(=O)c3ccccc3)cc2C1=O ; NCCOC(=O)c1ccc(-c2ccccc2)cc1N1C(=O)c2ccc(-c3c[nH]nn3)cc2C1=O ; COc1ccc(-c2nnn[nH]2)c(N2C(=O)c3ccc(-c4c[nH]nn4)cc3C2=O)c1 ; CN1CCN(CCOC(=O)c2ccc(-c3ccc(F)c(F)c3)cc2N2Cc3ccc(CO)cc3C2=O)CC1] 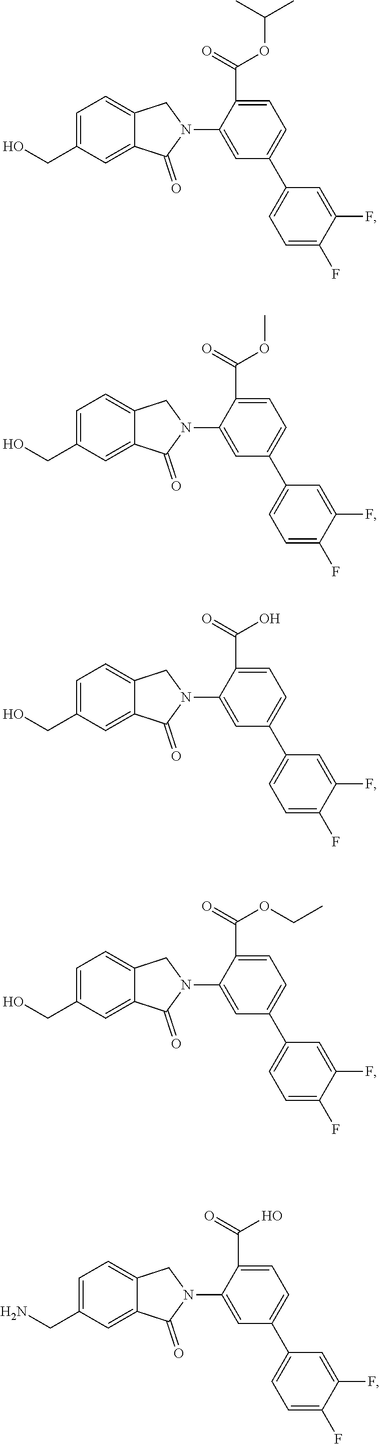 [SMILES: CC(C)OC(=O)c1ccc(-c2ccc(F)c(F)c2)cc1N1Cc2ccc(CO)cc2C1=O ; COC(=O)c1ccc(-c2ccc(F)c(F)c2)cc1N1Cc2ccc(CO)cc2C1=O ; O=C(O)c1ccc(-c2ccc(F)c(F)c2)cc1N1Cc2ccc(CO)cc2C1=O ; CCOC(=O)c1ccc(-c2ccc(F)c(F)c2)cc1N1Cc2ccc(CO)cc2C1=O ; NCc1ccc2c(c1)C(=O)N(c1cc(-c3ccc(F)c(F)c3)ccc1C(=O)O)C2] 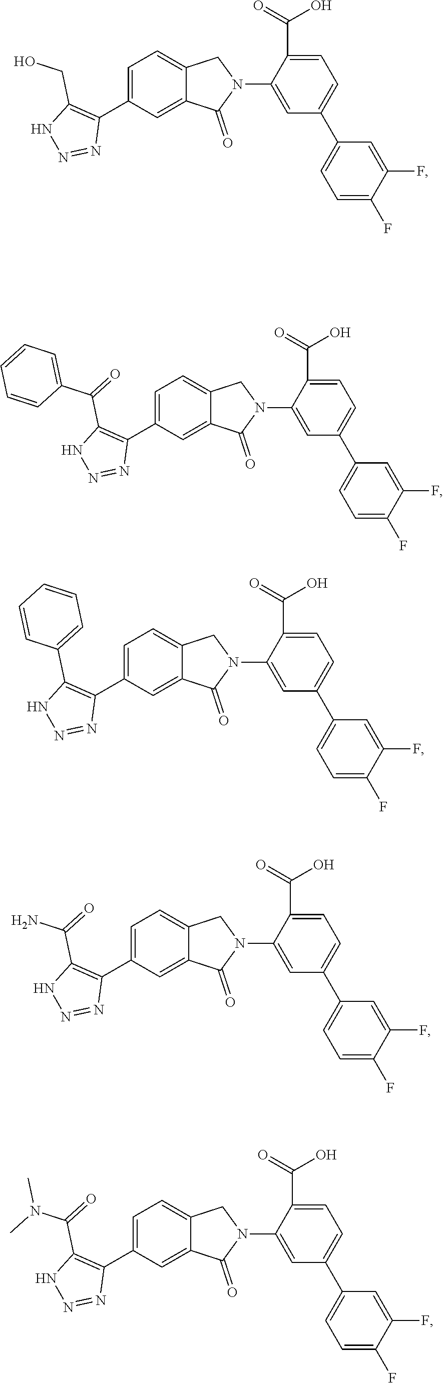 [SMILES: O=C(O)c1ccc(-c2ccc(F)c(F)c2)cc1N1Cc2ccc(-c3nn[nH]c3CO)cc2C1=O ; O=C(O)c1ccc(-c2ccc(F)c(F)c2)cc1N1Cc2ccc(-c3nn[nH]c3C(=O)c3ccccc3)cc2C1=O ; O=C(O)c1ccc(-c2ccc(F)c(F)c2)cc1N1Cc2ccc(-c3nn[nH]c3-c3ccccc3)cc2C1=O ; NC(=O)c1[nH]nnc1-c1ccc2c(c1)C(=O)N(c1cc(-c3ccc(F)c(F)c3)ccc1C(=O)O)C2 ; CN(C)C(=O)c1[nH]nnc1-c1ccc2c(c1)C(=O)N(c1cc(-c3ccc(F)c(F)c3)ccc1C(=O)O)C2] 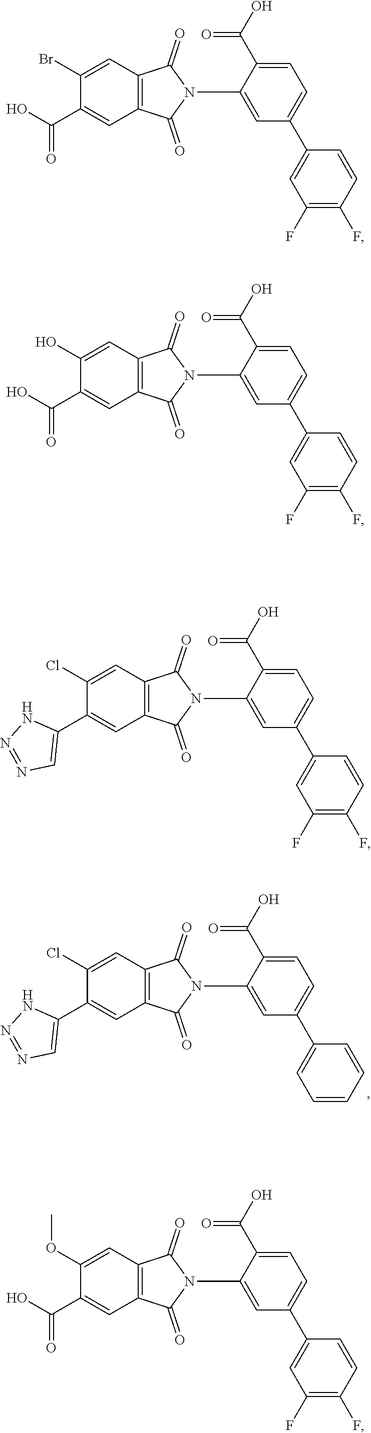 [SMILES: O=C(O)c1cc2c(cc1Br)C(=O)N(c1cc(-c3ccc(F)c(F)c3)ccc1C(=O)O)C2=O ; O=C(O)c1cc2c(cc1O)C(=O)N(c1cc(-c3ccc(F)c(F)c3)ccc1C(=O)O)C2=O ; O=C(O)c1ccc(-c2ccc(F)c(F)c2)cc1N1C(=O)c2cc(Cl)c(-c3cnn[nH]3)cc2C1=O ; O=C(O)c1ccc(-c2ccccc2)cc1N1C(=O)c2cc(Cl)c(-c3cnn[nH]3)cc2C1=O ; COc1cc2c(cc1C(=O)O)C(=O)N(c1cc(-c3ccc(F)c(F)c3)ccc1C(=O)O)C2=O] 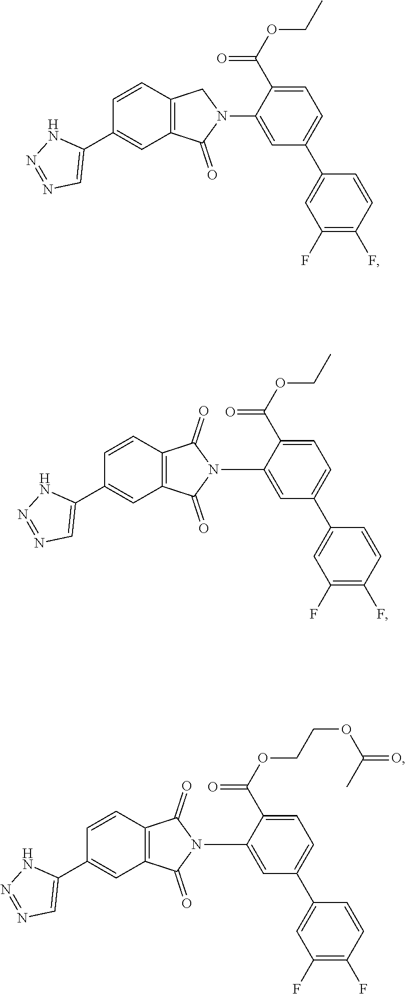 [SMILES: CCOC(=O)c1ccc(-c2ccc(F)c(F)c2)cc1N1Cc2ccc(-c3cnn[nH]3)cc2C1=O ; CCOC(=O)c1ccc(-c2ccc(F)c(F)c2)cc1N1C(=O)c2ccc(-c3cnn[nH]3)cc2C1=O ; CC(=O)OCCOC(=O)c1ccc(-c2ccc(F)c(F)c2)cc1N1C(=O)c2ccc(-c3cnn[nH]3)cc2C1=O]
or a pharmaceutically acceptable salt thereof.

4. A compound of claim 1, wherein the compound is selected from the group consisting of: 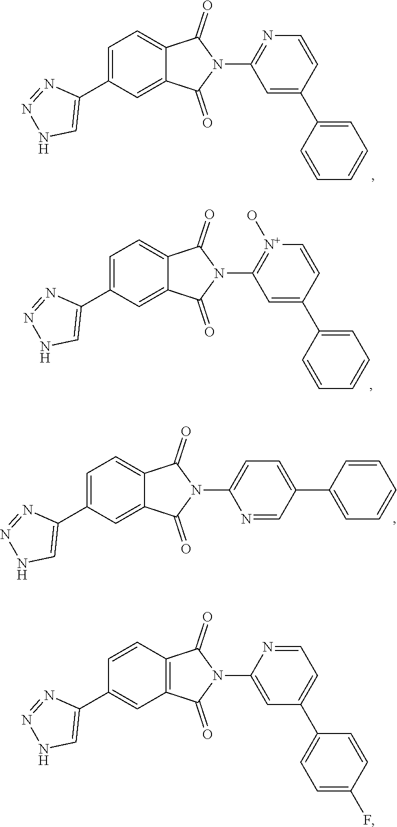 [SMILES: O=C1c2ccc(-c3c[nH]nn3)cc2C(=O)N1c1cc(-c2ccccc2)ccn1 ; O=C1c2ccc(-c3c[nH]nn3)cc2C(=O)N1c1cc(-c2ccccc2)cc[n+]1O ; O=C1c2ccc(-c3c[nH]nn3)cc2C(=O)N1c1ccc(-c2ccccc2)cn1 ; O=C1c2ccc(-c3c[nH]nn3)cc2C(=O)N1c1cc(-c2ccc(F)cc2)ccn1]
or a pharmaceutically acceptable salt thereof.

5. A compound of claim 1, wherein compound is presented by the compound of Formula (II): 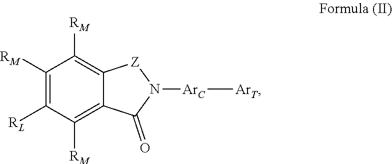
a prodrug thereof, a pharmaceutically acceptable salt thereof, or combination thereof, wherein: Z is —C(═O)— or —C(R^{a})(R^{b})—;
R^{a} and R^{b} are each independently selected from hydrogen, halogen, —OH, C₁₋₆ alkyl, C₁₋₆ alkoxy;
Ar_{C} is selected from arylene and heteroarylene; wherein Ar_{C} is substituted with one or more R_{C};
each R_{C} is independently selected from halogen, —CN, optionally substituted C₁-C₆ alkyl, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;
wherein the C₁-C₆ alkyl is optionally substituted with one or more substituents independently selected from halogen, —C(═O)OR⁷, —C(═O)NR^{1T}R², —OH, aryl, heteroaryl, C₁-C₆ alkoxy, C₃-C₈ cycloalkyl, and C₂-C₈ heterocycloalkyl; and
wherein the aryl and heteroaryl are optionally substituted with one or more substituents independently selected from —OH, halogen, —C(═O)OR⁷, —C(═O)NR¹R², C₁-C₆ alkyl, C₁-C₆ alkoxy, and —NR⁷R⁸;
each R¹ and R² is independently selected from hydrogen and C₁-C₆ alkyl optionally substituted with one or more substituents independently selected from halogen, —C(═O)OR⁷, —C(═O)NR⁷R⁸, —OH, aryl, heteroaryl, C₃-C₈ cycloalkyl, and C₂-C₈ heterocycloalkyl;
or R¹ and R² are taken together with the N to which they are attached to form a C₂-C₈ heterocycloalkyl optionally substituted with one or more C₁-C₆ alkyl substituents;
each R³ is independently C₁-C₆ alkyl optionally substituted with one or more substituents independently selected from —OH, optionally substituted —OC(═O)C₁-C₆ alkyl, optionally substituted —C(═O)OC₁-C₆ alkyl, C₁-C₆ alkoxy, —C(═O)OH, —NR¹R²;
wherein the —OC(═O)C₁-C₆ alkyl and —C(═O)OC₁-C₆ alkyl are optionally substituted with one or more substituents independently selected from with —OH or —NR⁷R⁸;
each R⁴ and R⁵ is independently selected from hydrogen and C₁-C₆ alkyl optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl;
or R⁴ and R⁵ are taken together with the N to which they are attached to form a C₂-C₈ heterocycloalkyl optionally substituted with one or more C₁-C₆ alkyl substituents;
each R⁶ are independently selected from optionally substituted C₁-C₆ alkyl and optionally substituted aryl;
wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and
wherein the aryl is optionally substituted with one or more substituents independently selected from —OH, halogen, —C(═O)OR⁷, —C(═O)NR⁷R⁸, C₁-C₆ alkyl, C₁-C₆ alkoxy, and —NR⁷R⁸;
each R⁷ and R⁸ is independently selected from hydrogen and C₁-C₆ alkyl;
or R⁷ and R⁸ are taken together with the N to which they are attached to form an optionally substituted C₂-C₅ heterocycloalkyl optionally substituted with one or more C₁-C₆ alkyl substituents;
Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;
each R_{M} is independently selected from hydrogen, halogen, —OH, —CN, optionally substituted C₁-C₆ alkyl, and optionally substituted C₁-C₆ alkoxy;
wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₃-C₈ cycloalkyl, —O—C₃-C₈ cycloalkyl, C₂-C₈ heterocycloalkyl, and —O—C₂-C₈ heterocycloalkyl;
R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;
wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR⁷R⁸, —C(═O)R¹², aryl, or C₁-C₆ alkyl-(aryl);
R⁹ is C₁-C₆ alkyl optionally substituted with one or more substituents independently selected from —OH and —NR¹R²;
each R¹⁰ and R¹¹ is independently selected from hydrogen and C₁-C₆ alkyl optionally substituted with one or more substituents independently selected from —C(═O)OR⁷, —C(═O)NR¹R², —OH, aryl, hydroxyaryl or heteroaryl;
or R¹⁰ and R¹¹ are taken together with the N to which they are attached to form a C₂-C₈ heterocycloalkyl optionally substituted with one or more C₁-C₆ alkyl substituents;
R¹² is selected from C₁-C₆ alkyl and aryl optionally substituted with one or more C₁-C₆ alkyl substituents; and wherein at least one R_{C} is —C(═O)OH; or R_{L} is —C(═O)OH.

6. The compound of claim 1, wherein the Formula (I) is represented by Formula (III): 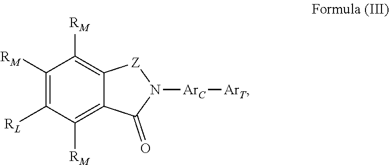
or a pharmaceutically acceptable salt thereof, wherein: Z is —C(═O)— or —C(R^{a})(R^{b})—;
R^{a} and R^{b} are each independently selected from hydrogen, halogen, —OH, C₁₋₆ alkyl, C₁₋₆ alkoxy;
Ar_{C} is selected from arylene and heteroarylene; wherein Ar_{C} is substituted with one or more R_{C};
each R_{C} is independently selected from halogen, —CN, optionally substituted C₁-C₆ alkyl, optionally substituted heteroaryl, optionally substituted aryl, —C(═O)OH, —C(═O)OR³, —C(═O)NR⁴R⁵, —S(═O)₂NR⁴R⁵, —NHS(═O)₂R⁶, and —C(═O)NHS(═O)₂R⁶;
wherein the C₁-C₆ alkyl is optionally substituted with one or more substituents independently selected from halogen, —C(═O)OR⁷, —C(═O)NR¹R², —OH, aryl, heteroaryl, C₁-C₆alkoxy, C₃-C₈ cycloalkyl, and C₂-C₈ heterocycloalkyl; and
wherein the aryl and heteroaryl are optionally substituted with one or more substituents independently selected from —OH, halogen, —C(═O)OR⁷, —C(═O)NR¹R², C₁-C₆ alkyl, C₁-C₆ alkoxy, and —NR⁷R⁸;
each R¹ and R² is independently selected from hydrogen and C₁-C₆ alkyl optionally substituted with one or more substituents independently selected from halogen, —C(═O)OR⁷, —C(═O)NR⁷R⁸, —OH, aryl, heteroaryl, C₃-C₈ cycloalkyl, and C₂-C₈ heterocycloalkyl;
or R¹ and R² are taken together with the N to which they are attached to form a C₂-C₈ heterocycloalkyl optionally substituted with one or more C₁-C₆ alkyl substituents;
each R³ is independently C₁-C₆ alkyl optionally substituted with one or more substituents independently selected from —OH, optionally substituted —OC(═O)C₁-C₆ alkyl, optionally substituted —C(═O)OC₁-C₆ alkyl, C₁-C₆ alkoxy, —C(═O)OH, —NR¹R²;
wherein the —OC(═O)C₁-C₆ alkyl and —C(═O)OC₁-C₆ alkyl are optionally substituted with one or more substituents independently selected from with —OH or —NR⁷R⁸;
each R⁴ and R⁵ is independently selected from hydrogen and C₁-C₆ alkyl optionally substituted with one or more substituents independently selected from halogen, —C(═O)OH, —C(═O)NR¹R², —OH, aryl, heteroaryl, C₃-C₈ cycloalkyl, and C₂-C₈ heterocycloalkyl;
or R⁴ and R⁵ are taken together with the N to which they are attached to form a C₂-C₈ heterocycloalkyl optionally substituted with one or more C₁-C₆ alkyl substituents;
R⁶ is selected from optionally substituted C₁-C₆ alkyl and optionally substituted aryl;
wherein the alkyl is optionally substituted with one or more substituents independently selected from halogen, —C(═O)OR⁷, —C(═O)NR¹R², —OH, aryl, heteroaryl, C₃-C₈ cycloalkyl, and C₂-C₈ heterocycloalkyl; and
wherein the aryl is optionally substituted with one or more substituents independently selected from —OH, halogen, —C(═O)OR⁷, —C(═O)NR⁷R⁸, C₁-C₆ alkyl, C₁-C₆ alkoxy, and —NR⁷R⁸;
each R⁷ and R⁸ is independently selected from hydrogen and C₁-C₆ alkyl;
or R⁷ and R⁸ are taken together with the N to which they are attached to form an optionally substituted C₂-C₈ heterocycloalkyl optionally substituted with one or more C₁-C₆ alkyl substituents;
Ar_{T} is selected from pyridinyl, pyrimidinyl, pyrazinyl, phenyl, thiophenyl, pyrazolyl, and imidazolyl, wherein Ar_{T} is optionally substituted by one or more substituents selected from halogen, —OH, —NR⁷R⁸, —CN, C₁-C₆ alkyl, and C₁-C₆ alkoxy;
each R_{M} is independently selected from hydrogen, halogen, —OH, —CN, optionally substituted C₁-C₆ alkyl, and optionally substituted C₁-C₆ alkoxy;
wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₃-C₈ cycloalkyl, —O—C₃-C₈ cycloalkyl, C₂-C₈ heterocycloalkyl, and —O—C₂-C₈ heterocycloalkyl;
R_{L} is selected from optionally substituted heteroaryl, —C(═O)OH, —C(═O)OR⁹, —C(═O)NR¹⁰R¹¹, —NHC(═O)R¹², —NHS(═O)₂R¹², or —C(═O)NHS(═O)₂R¹²;
wherein the heteroaryl is optionally substituted with one or more substituents independently selected from C₁-C₆ alkyl, —OC(═O)C₁-C₆ alkyl, (C₁-C₄ alkylene)-O—C(═O)C₁-C₆ alkyl, —C(═O)NR⁷R⁸, —C(═O)R¹², aryl, or C₁-C₆ alkyl-(aryl);
R⁹ is C₁-C₆ alkyl optionally substituted with one or more substituents independently selected from —OH and —NR¹R²;
each R¹⁰ and R¹¹ is independently selected from hydrogen and C₁-C₆ alkyl optionally substituted with one or more substituents independently selected from —C(═O)OH, —C(═O)NR¹R², —OH, aryl, hydroxyaryl or heteroaryl;
or R¹⁰ and R¹¹ are taken together with the N to which they are attached to form a C₂-C₈ heterocycloalkyl optionally substituted with one or more C₁-C₆ alkyl substituents;
R¹² is selected from C₁-C₆ alkyl and aryl optionally substituted with one or more C₁-C₆ alkyl substituents; and wherein at least one R_{C} is —C(═O)OR³ or R_{L} is —C(═O)OR⁹.

7. The compound of claim 1, wherein the Formula (I) is represented by Formula (IV): 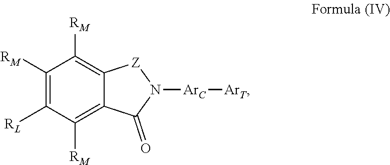
or a pharmaceutically acceptable salt thereof, wherein: Z is —C(═O)— or —C(R^{a})(R^{b})—;
R^{a} and R^{b} are each independently selected from hydrogen, fluorine and methyl;
Ar_{C} is selected from arylene and heteroarylene; each substituted with one or more R_{C};
R_{C} is selected from —CN, —OH, C₁-C₆ alkoxy, C₁-C₆ alkyl, C₁-C₆ hydroxyalkyl, heteroaryl, aryl, —C(═O)OH, and —C(═O)OR³;
each R¹ and R² is independently selected from hydrogen and C₁-C₆ alkyl;
or R¹ and R² are taken together with the N to which they are attached to form a C₂-C₈ heterocycloalkyl;
each R³ is independently C₁-C₆ alkyl optionally substituted with one or more —NR¹R² or C₁-C₆ alkoxy;
Ar_{T} is selected from pyridinyl, phenyl, thiophenyl, pyrazolyl, imidazolyl, and tetrazolyl, wherein Ar_{T} is optionally substituted by one or more substituents independently selected from halogen, —NR⁷R⁸, C₁-C₆ alkyl, and C₁-C₆ alkoxy;
each R_{M} is independently selected from hydrogen and halogen;
R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one or more substituents independently selected from (C₁-C₄ alkylene)-O—C(═O)C₁-C₆ alkyl, —C(═O)NR¹R², —C(═O)R¹², aryl, and C₁-C₆ alkyl-(aryl);
each R¹⁰ and R¹¹ is independently selected from hydrogen and C₁-C₆ alkyl optionally substituted with one or more of —C(═O)OH, —OH, aryl, hydroxyaryl, or heteroaryl; and
R¹² is selected from C₁-C₆ alkyl and aryl.

8. The compound of claim 1, wherein Formula (I) is represented by Formula (V): 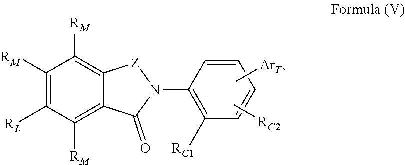
or a pharmaceutically acceptable salt thereof,
wherein:
Z is —C(═O)— or —C(R^{a})(R^{b})—;
R^{a} and R^{b} are each independently selected from hydrogen, fluorine and methyl;
R_{C1} is selected from —OH, tetrazolyl, —C(═O)OH, and —C(═O)OR³;
R_{C2} is selected from hydrogen, halogen, —OH, C₁-C₆ alkyl, C₁-C₆ hydroxyalkyl and C₁-C₆ alkoxy;
R³ is C₁-C₆ alkyl optionally substituted with one or more substituent selected from —NR¹R² or C₁-C₆ alkoxy;
each R¹ and R² is independently selected from hydrogen and C₁-C₆ alkyl;
or R¹ and R² are taken together with the N to which they are attached to form a C₂-C₈ heterocycloalkyl;
Ar_{T} is selected from pyridinyl, phenyl, thiophenyl, pyrazolyl, imidazolyl, and tetrazolyl, wherein Ar_{T} is optionally substituted by one or more substituents independently selected from halogen, C₁-C₆ alkyl, and C₁-C₆ alkoxy;
each R_{M} is independently selected from hydrogen and halogen;
R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;
R¹⁰ is selected from hydrogen and C₁-C₆ alkyl;
R¹¹ is selected from hydrogen and optionally substituted C₁-C₆ alkyl; wherein the C₁-C₆alkyl is optionally substituted with one or more of —C(═O)OH, —OH, aryl, hydroxyaryl, and heteroaryl; and
R¹² is selected from C₁-C₆ alkyl and aryl.

9. The compound of claim 1, wherein the compound of Formula (I) is represented by the compound of Formula (VI): 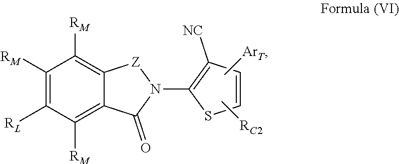
or a pharmaceutically acceptable salt thereof,
wherein:
Z is —C(═O)— or —C(R^{a})(R^{b})—;
R^{a} and R^{b} are each independently selected from hydrogen, fluorine and methyl;
R_{C2} is selected from hydrogen, halogen, —OH, C₁-C₆ alkyl, C₁-C₆ hydroxyalkyl, C₁-C₆ alkoxy and aryl;
Ar_{T} is phenyl optionally substituted by one or more substituents independently selected from halogen, C₁-C₆ alkyl, and C₁-C₆ alkoxy;
each R_{M} is independently selected from hydrogen and halogen;
R_{L} is selected from optionally substituted heteroaryl, —C(═O)OH, —C(═O)NR¹⁰R¹¹, and —C(═O)NHS(═O)₂R¹²; wherein the heteroaryl is optionally substituted by one of —C(═O)R¹² or aryl;
R¹⁰ is selected from hydrogen and C₁-C₆ alkyl;
R¹¹ is selected from hydrogen and optionally substituted C₁-C₆ alkyl; wherein the C₁-C₆alkyl is optionally substituted with one or more of —C(═O)OH, —OH, aryl, hydroxyaryl, and heteroaryl; and
R¹² is selected from C₁-C₆ alkyl and aryl.

10. The compound of claim 1, wherein the Formula (I) is represented by Formula (VII): 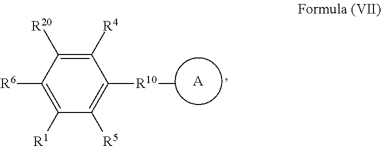
or a pharmaceutically acceptable salt thereof,
wherein:
A is selected from: 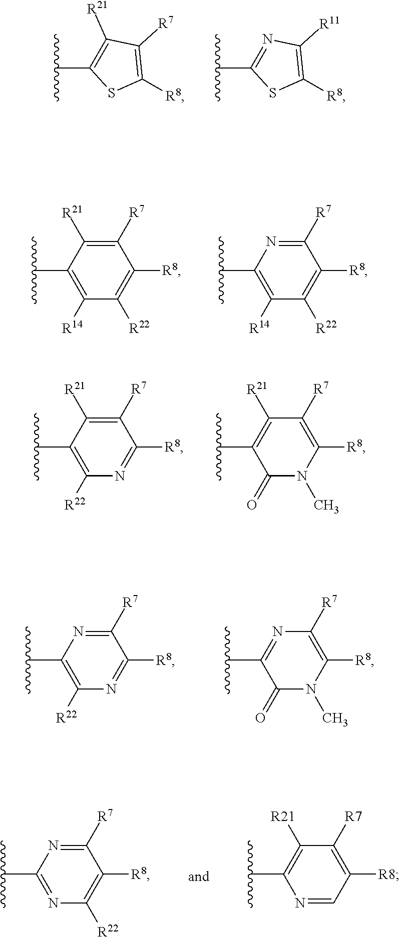
R¹ is selected from hydrogen, halogen, hydroxyl, C₁-C₆ alkyl, and C₁-C₆ alkoxy,
wherein the C₁-C₆ alkyl and C₁-C₆ alkoxy are optionally substituted with one or more halogens;
each R² and R³ is independently selected from hydrogen and C₁-C₆ alkyl,
wherein the C₁-C₆ alkyl is optionally substituted with one or more halogens;
or R² and R³ are taken together with the N to which they are attached to form a 3- to 10-membered heterocycle optionally substituted with one or more substituents independently selected from C₁-C₆ alkyl;
R⁴ is selected from hydrogen, halogen, C₁-C₆ alkyl, and C₁-C₆ alkoxy,
wherein the C₁-C₆ alkyl and C₁-C₆ alkoxy are optionally substituted with one or more substituents independently selected from halogens;
R⁵ is selected from —C(═O)OR¹⁵, —C(═O)NR²R³, —S(═O)₂NR²R³, —C(═O)NHR¹⁵, —CH₂OH, 3-hydroxyoxetan-3-yl, and —NH₂;
R⁶ is selected from hydrogen, halogen, hydroxyl, 5-membered heteroaryl, C₁-C₆ alkyl, —C(═O)OR¹⁵, —C(═O)R¹², —C(═O)NHR¹⁵, and —C(═O)N═S(═X³)(CH₃)₂,
wherein the C₁-C₆ alkyl are optionally substituted with one or more R⁹, and
wherein 5-membered heteroaryl contains at least two heteroatoms and is optionally substituted with one or more substituents independently selected from R¹⁷;
R⁷ is selected from hydrogen, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,
wherein the C₁-C₆ alkyl and C₁-C₆ alkoxy are optionally substituted with one or more halogens, and
wherein C₃-C₈ cycloalkyl, —O—C₃-C₈ cycloalkyl, 3- to 10-membered heterocycloalkyl, —O-(3- to 10-membered heterocycloalkyl), aryl and heteroaryl are optionally substituted with one or more R²⁴;
R⁸ is selected from hydrogen, —NO₂, C₁-C₆ alkyl, aryl, and heteroaryl,
wherein the C₁-C₆ alkyl and C₁-C₆ alkoxy are optionally substituted with one or more substituents independently selected at each occurrence from halogen; and
wherein aryl and heteroaryl are optionally substituted with one or more substituents independently selected at each occurrence from R²³;
or R⁷ and R⁸ are taken together to form a C₅-C₁₀ carbocycle or 5- to 10-membered heterocycle,
wherein C₅-C₁₀ carbocycle and 5- to 10-membered heterocycle are optionally substituted with one or more substituents independently selected from halogen, hydroxyl, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,
wherein the C₁-C₆ alkyl and C₁-C₆ alkoxy are optionally substituted with one or more halogens, and
wherein aryl, heteroaryl, C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;
each R⁹ is independently selected from hydroxy and —COOH;
R¹⁰ is selected from —C(═O)—X¹—, —CH₂—X¹—, —X¹—C(═O)—, and —X¹-CH₂—;
R¹¹ is selected from hydrogen, —NO₂, C₁-C₆ alkyl, C₁-C₆ alkoxy, C₃-C₅ cycloalkyl, —O—C₃-C₈ cycloalkyl, 3- to 10-membered heterocycloalkyl, and —O—(3- to 10-membered heterocycloalkyl),
wherein the C₁-C₆ alkyl and C₁-C₆ alkoxy are optionally substituted with one or more halogens, and
wherein C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;
R¹² is selected from alanine, arginine, asparagine, aspartic acid, asparagine, aspartic acid, cysteine, glutamic acid, glutamine, glutamine, glutamic acid, glycine, histidine, isoleucine, leucine, lysine, methionine, phenylalanine, proline, serine, threonine, tryptophan, tyrosine, and valine, wherein the point of attachment of R¹² is a nitrogen atom;
R¹⁴ is selected from hydrogen, halogen, hydroxyl, nitrile, —C(═O)CR¹⁵ and —C(═O)OR¹⁵;
each R¹⁵ is independently selected from hydrogen and C₁-C₆ alkyl, -heterocyclyl,
wherein the C₁-C₆ alkyl is optionally substituted with one or more substituents independently selected at each occurrence from —C(═O)NR²R³, -heterocyclyl, —NR²R³;
wherein the heterocyclyl is optionally substituted with one or more substituents independently selected at each occurrence from R² and R³;
R¹⁷ is selected from C₁-C₆ alkyl, aryl, and 6-membered heteroaryl,
wherein the C₁-C₆ alkyl is optionally substituted with one or more hydroxy, and
wherein aryl and 6-membered heteroaryl are optionally substituted with one or more substituents independently selected from halogen, —R², and —OR²;
R²⁰ is selected from hydrogen, halogen, hydroxyl, —COOH, —NC(═O)R², —OR², 5-membered heteroaryl, C₁-C₆ alkyl, —C(═O)N═S(═X³)(CH₃)₂, —CH₂(OH)CH₂OH and —NH—SO₂—R²,
wherein the 5-membered heteroaryl contains at least two heteroatoms, and
wherein the C₁-C₆ alkyl is optionally substituted with one or more substituents independently selected from 5-membered heteroaryl, wherein the 5-membered heteroaryl contains at least two heteroatoms;
R²¹ is selected from hydrogen and nitrile;
R²² is selected from hydrogen and hydroxy;
each R²³ is independently selected from halogen, C₁-C₆ alkyl, C₁-C₆ alkoxy,
wherein the C₁-C₆ alkyl and C₁-C₆ alkoxy are optionally substituted with one or more substituents independently selected from halogens;
each R²⁴ is independently selected from halogen, C₁-C₆ alkyl, C₁-C₆ alkoxy, 5-membered heteroaryl wherein the C₁-C₆ alkyl and C₁-C₆ alkoxy are optionally substituted with one or more substituents independently selected from halogens;
each X¹ is independently selected from —NR²— and —CR²R³—; and
each X³ is independently selected from NH and O.

11. The compound or salt of claim 10
wherein R¹⁴ is selected from hydrogen and —C(═O)OR¹⁵, wherein R¹⁵ is selected from hydrogen and C₁-C₃ alkyl.

12. The compound or salt of claim 10, wherein R¹⁴ is selected from hydrogen and —C(═O)OR¹⁵,
wherein R¹⁵ is selected from hydrogen and C₁-C₃ alkyl.

13. The compound of claim 10, wherein R¹⁴ is —COOH.

14. The compound of claim 10, wherein the compound of Formula (VII) is selected from the group consisting of:

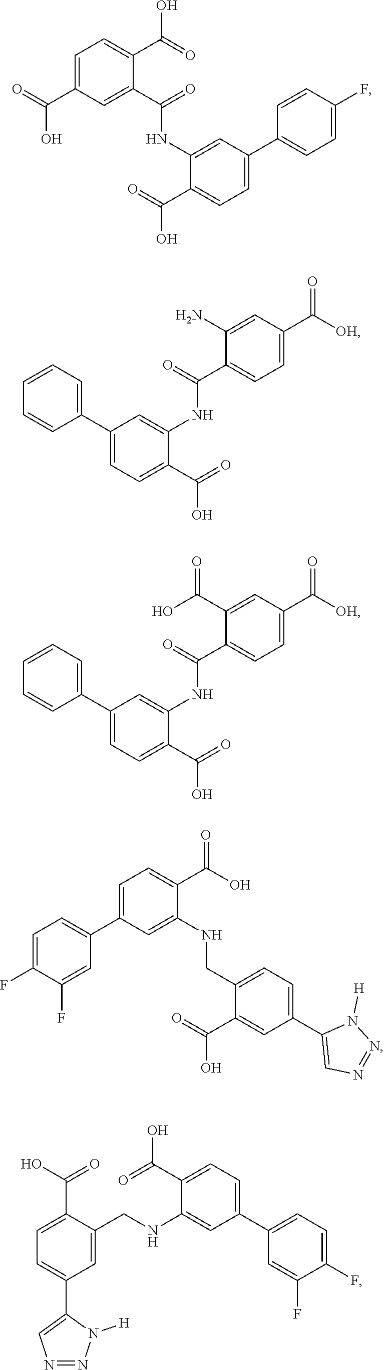

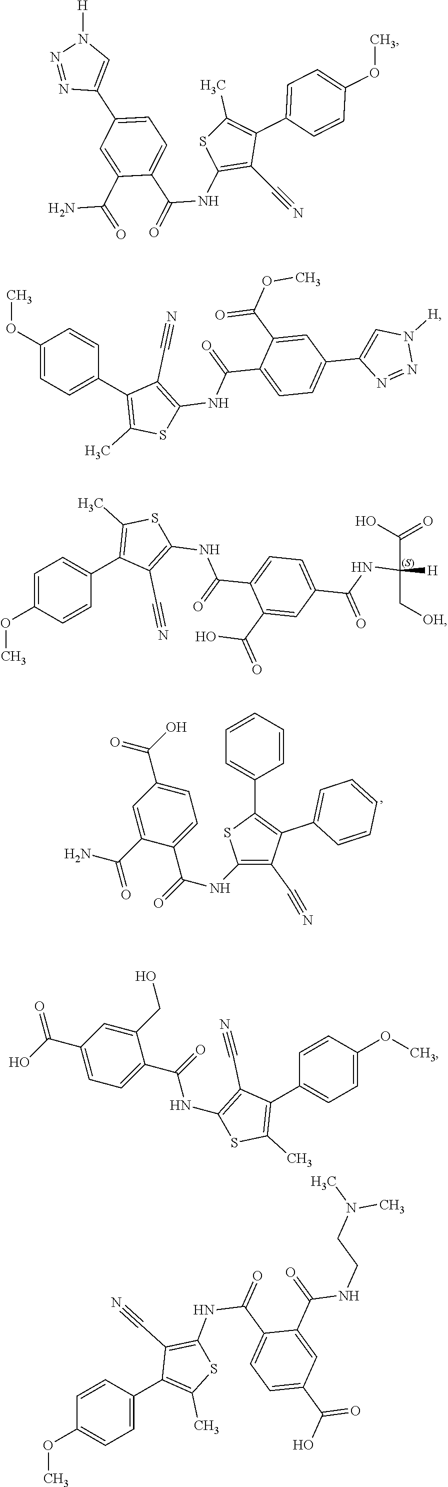

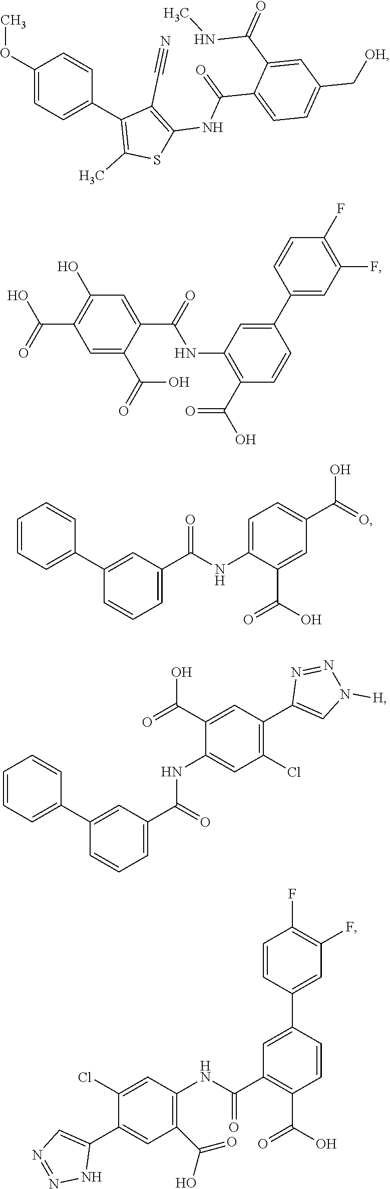

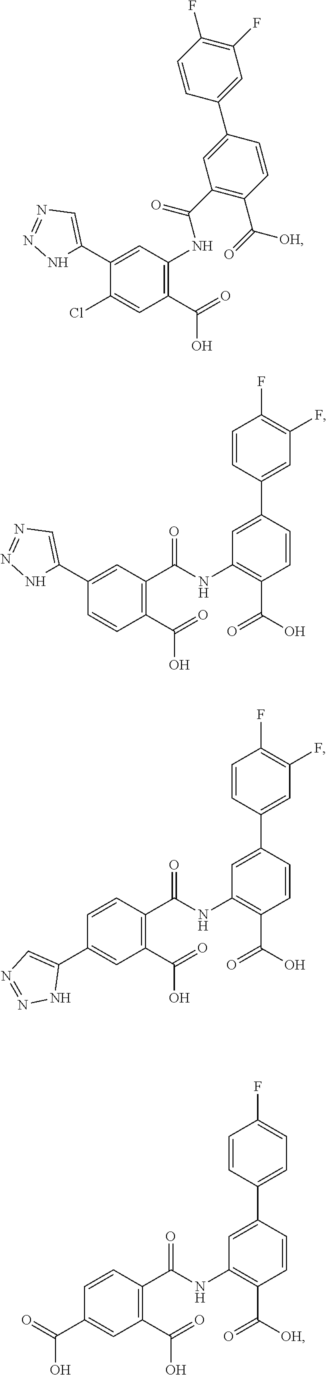 [SMILES: O=C(O)c1cc(Cl)c(-c2cnn[nH]2)cc1NC(=O)c1cc(-c2ccc(F)c(F)c2)ccc1C(=O)O ; O=C(O)c1ccc(-c2ccc(F)c(F)c2)cc1NC(=O)c1cc(-c2cnn[nH]2)ccc1C(=O)O ; O=C(O)c1ccc(-c2ccc(F)c(F)c2)cc1NC(=O)c1ccc(-c2cnn[nH]2)cc1C(=O)O ; O=C(O)c1ccc(C(=O)Nc2cc(-c3ccc(F)cc3)ccc2C(=O)O)c(C(=O)O)c1]

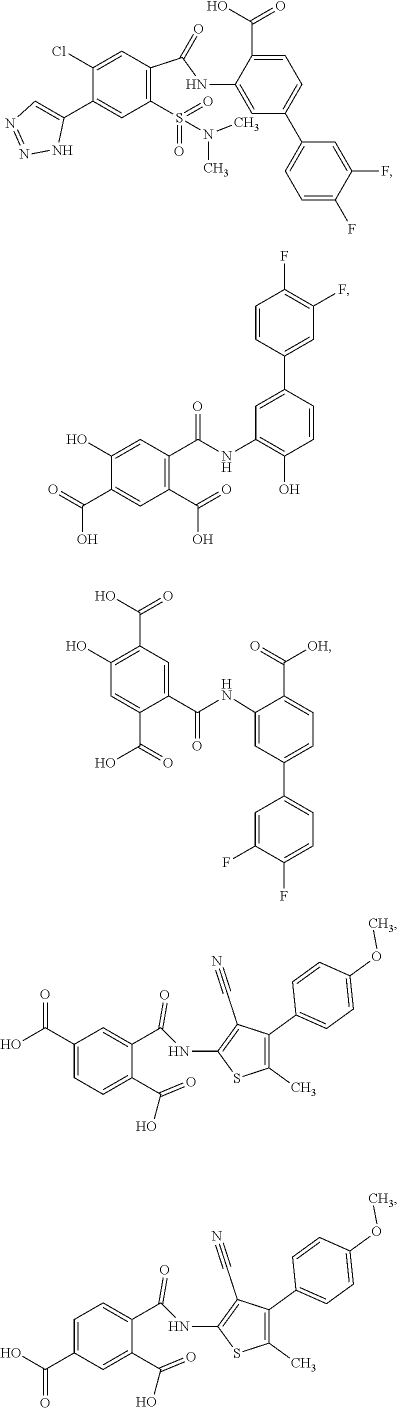 [SMILES: CN(C)S(=O)(=O)c1cc(-c2cnn[nH]2)c(Cl)cc1C(=O)Nc1cc(-c2ccc(F)c(F)c2)ccc1C(=O)O ; O=C(O)c1cc(C(=O)O)c(C(=O)Nc2cc(-c3ccc(F)c(F)c3)ccc2O)cc1O ; O=C(O)c1cc(C(=O)Nc2cc(-c3ccc(F)c(F)c3)ccc2C(=O)O)c(C(=O)O)cc1O ; COc1ccc(-c2c(C)sc(NC(=O)c3cc(C(=O)O)ccc3C(=O)O)c2C#N)cc1 ; COc1ccc(-c2c(C)sc(NC(=O)c3ccc(C(=O)O)cc3C(=O)O)c2C#N)cc1]

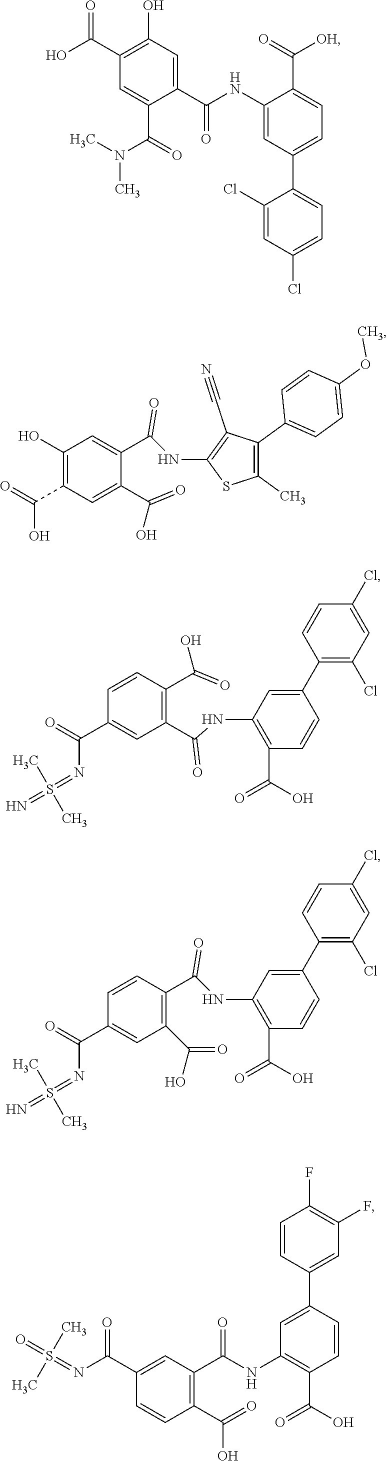

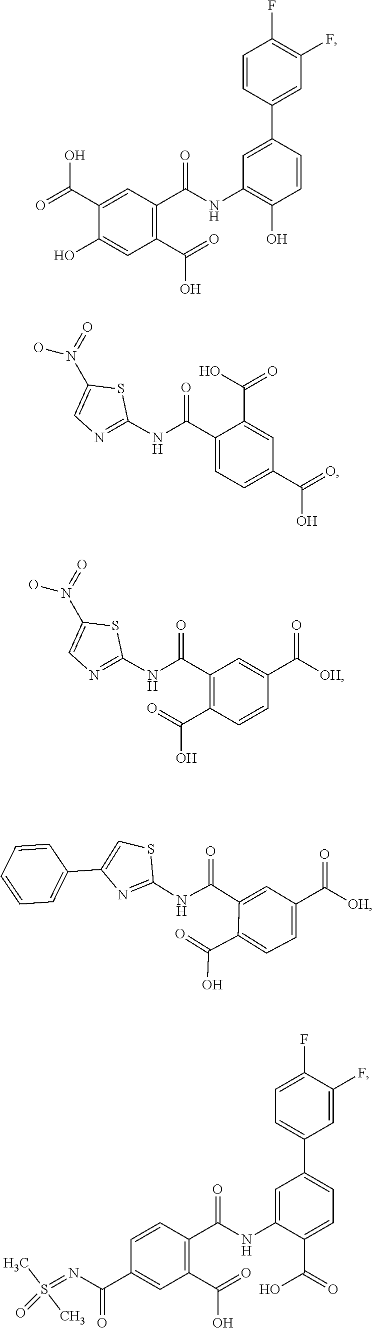

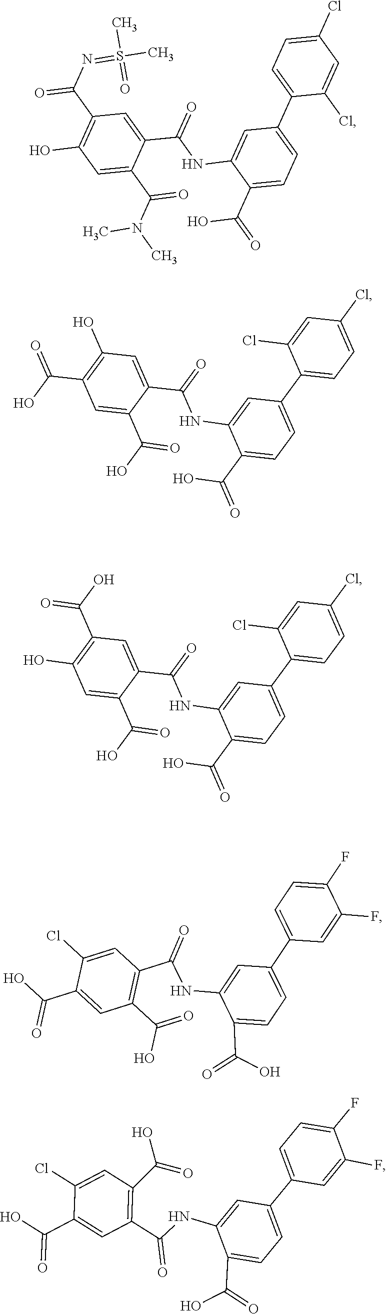 [SMILES: CN(C)C(=O)c1cc(O)c(C(=O)N=S(C)(C)=O)cc1C(=O)Nc1cc(-c2ccc(Cl)cc2Cl)ccc1C(=O)O ; O=C(O)c1cc(C(=O)O)c(C(=O)Nc2cc(-c3ccc(Cl)cc3Cl)ccc2C(=O)O)cc1O ; O=C(O)c1cc(C(=O)Nc2cc(-c3ccc(Cl)cc3Cl)ccc2C(=O)O)c(C(=O)O)cc1O ; O=C(O)c1cc(C(=O)O)c(C(=O)Nc2cc(-c3ccc(F)c(F)c3)ccc2C(=O)O)cc1Cl ; O=C(O)c1cc(C(=O)Nc2cc(-c3ccc(F)c(F)c3)ccc2C(=O)O)c(C(=O)O)cc1Cl]

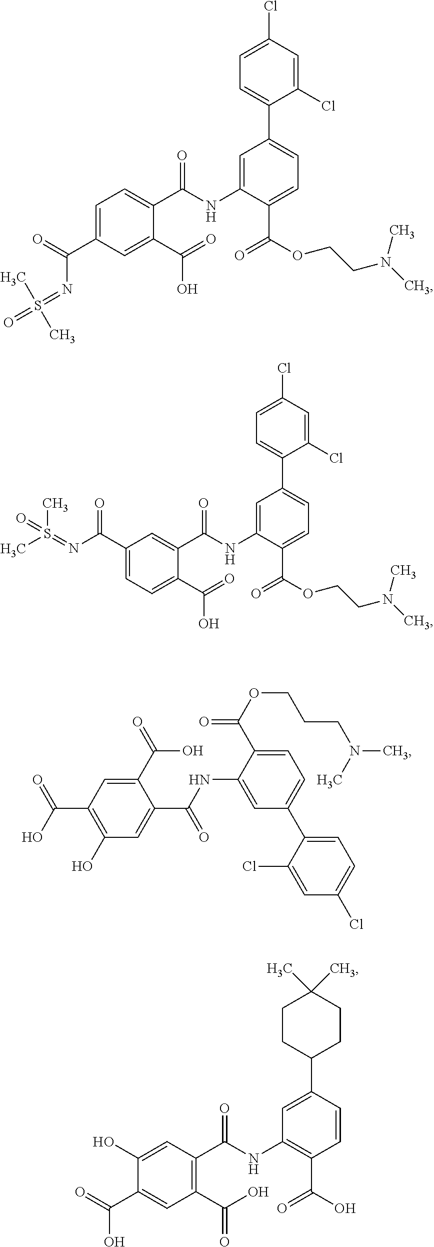 [SMILES: CN(C)CCOC(=O)c1ccc(-c2ccc(Cl)cc2Cl)cc1NC(=O)c1ccc(C(=O)N=S(C)(C)=O)cc1C(=O)O ; CN(C)CCOC(=O)c1ccc(-c2ccc(Cl)cc2Cl)cc1NC(=O)c1cc(C(=O)N=S(C)(C)=O)ccc1C(=O)O ; CN(C)CCCOC(=O)c1ccc(-c2ccc(Cl)cc2Cl)cc1NC(=O)c1cc(O)c(C(=O)O)cc1C(=O)O ; CC1(C)CCC(c2ccc(C(=O)O)c(NC(=O)c3cc(O)c(C(=O)O)cc3C(=O)O)c2)CC1]

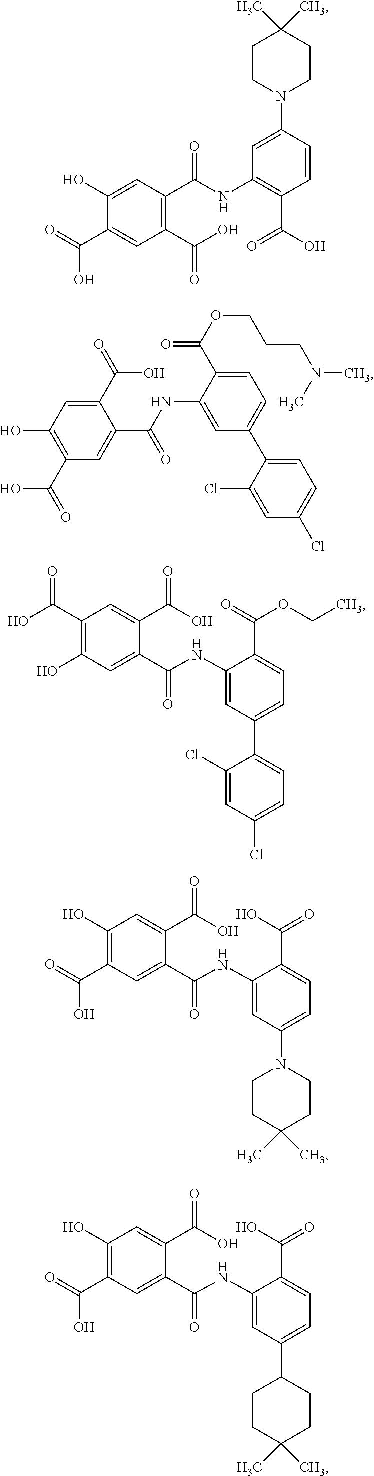

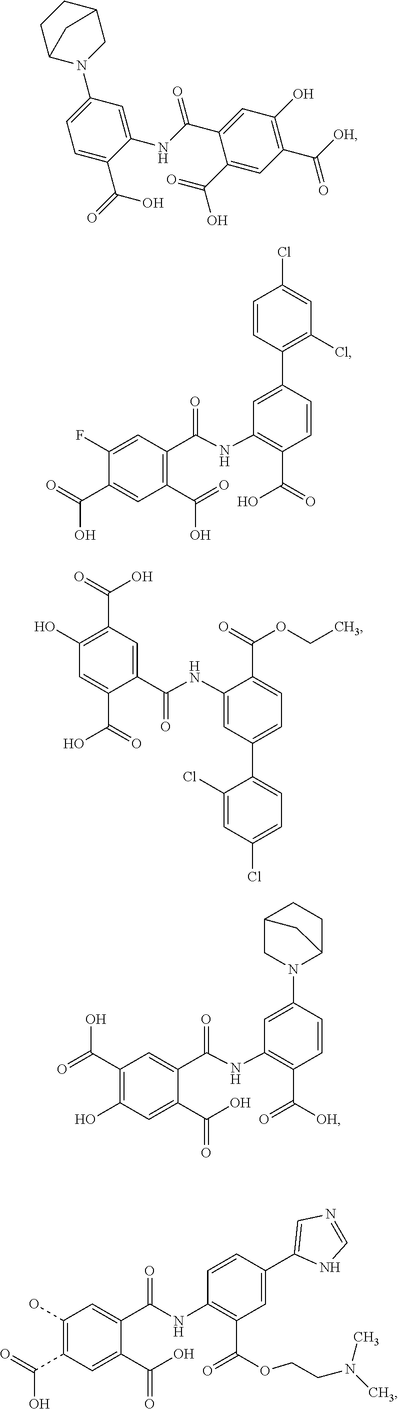

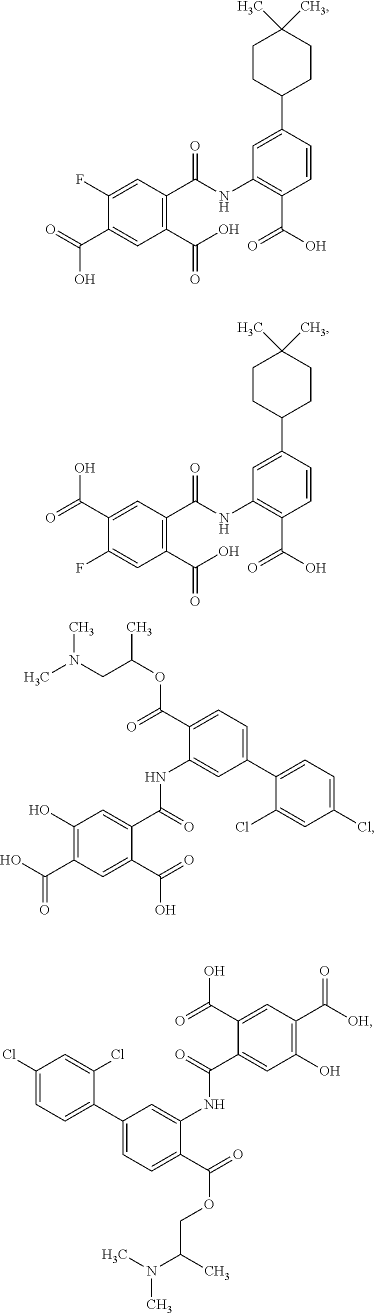 [SMILES: CC1(C)CCC(c2ccc(C(=O)O)c(NC(=O)c3cc(F)c(C(=O)O)cc3C(=O)O)c2)CC1 ; CC1(C)CCC(c2ccc(C(=O)O)c(NC(=O)c3cc(C(=O)O)c(F)cc3C(=O)O)c2)CC1 ; CC(CN(C)C)OC(=O)c1ccc(-c2ccc(Cl)cc2Cl)cc1NC(=O)c1cc(O)c(C(=O)O)cc1C(=O)O ; CC(COC(=O)c1ccc(-c2ccc(Cl)cc2Cl)cc1NC(=O)c1cc(O)c(C(=O)O)cc1C(=O)O)N(C)C]

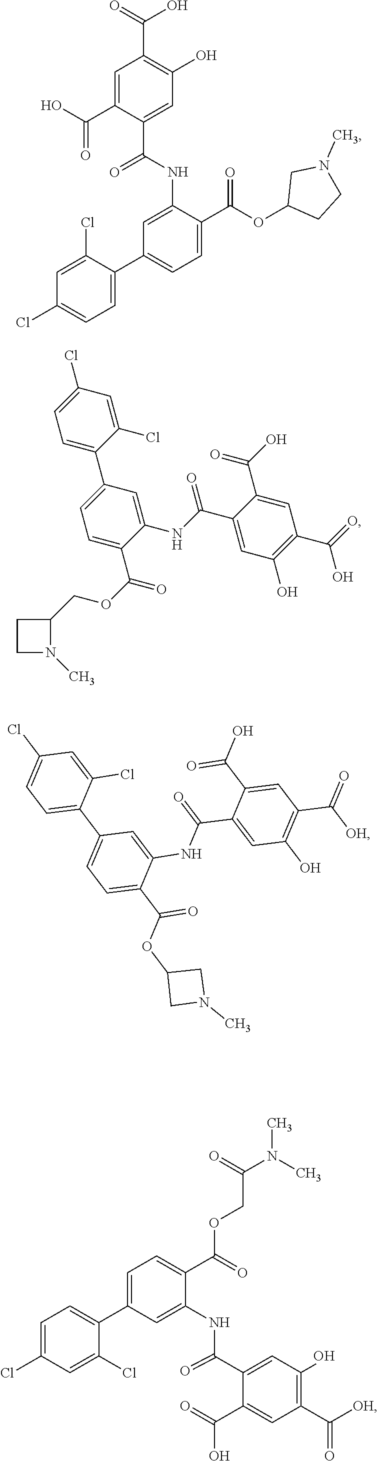

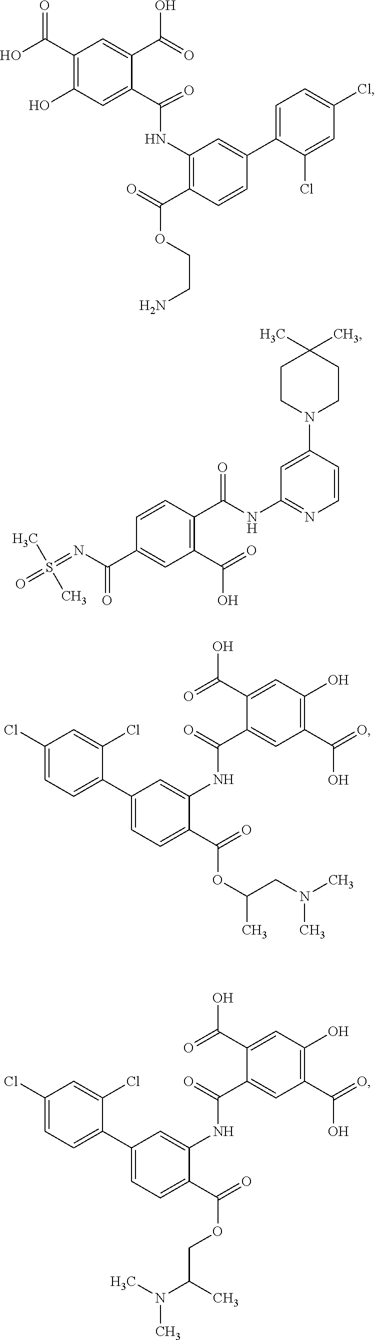

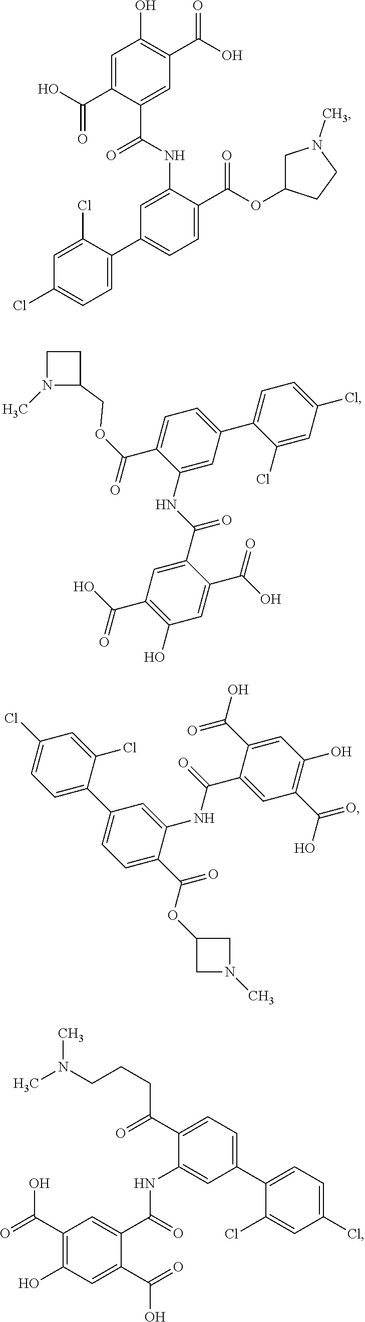

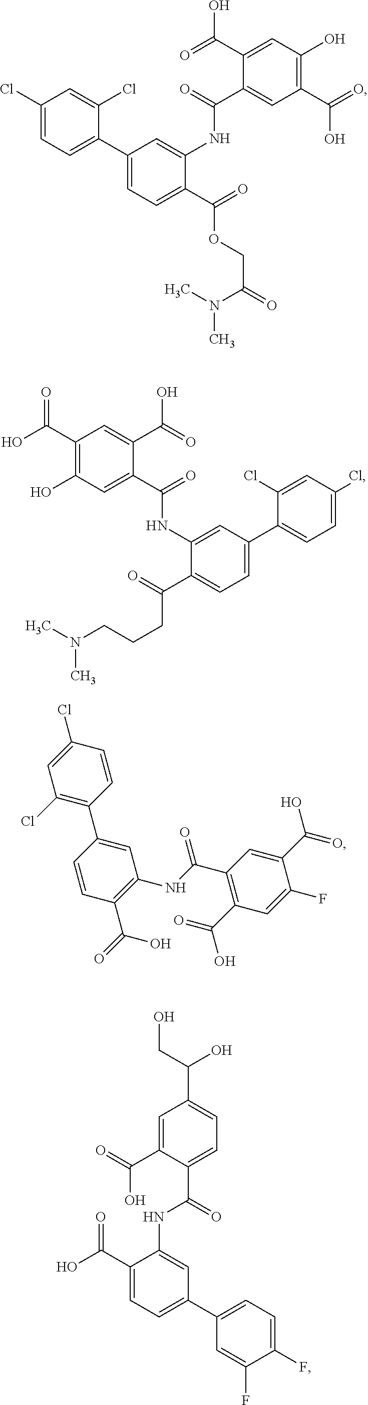

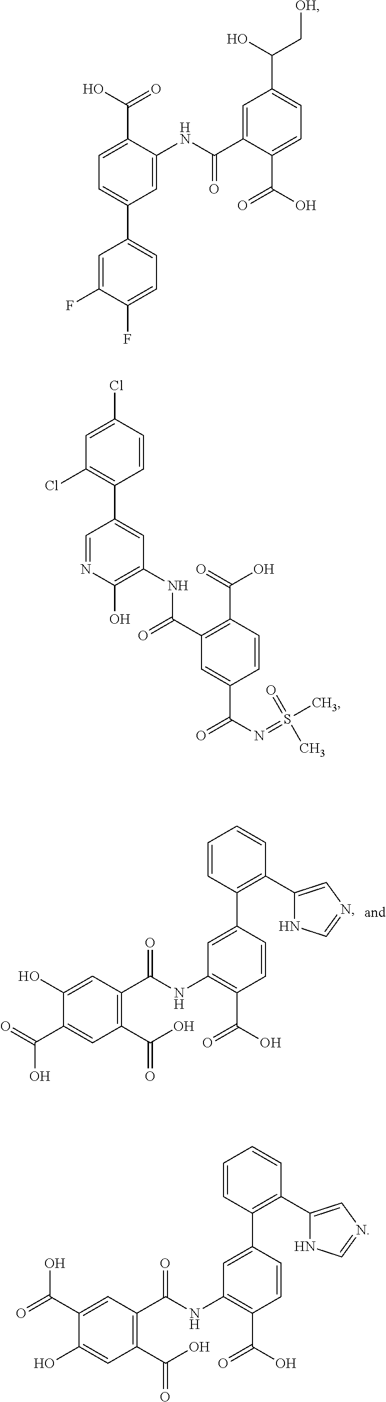 [SMILES: O=C(O)c1ccc(-c2ccc(F)c(F)c2)cc1NC(=O)c1cc(C(O)CO)ccc1C(=O)O ; CS(C)(=O)=NC(=O)c1ccc(C(=O)O)c(C(=O)Nc2cc(-c3ccc(Cl)cc3Cl)cnc2O)c1 ; O=C(O)c1cc(C(=O)O)c(C(=O)Nc2cc(-c3ccccc3-c3cnc[nH]3)ccc2C(=O)O)cc1O ; O=C(O)c1cc(C(=O)Nc2cc(-c3ccccc3-c3cnc[nH]3)ccc2C(=O)O)c(C(=O)O)cc1O]

15. A method of treatment or preventing disease, disorder or condition, comprising reducing, binding,
inhibiting or degrading of PFKFB3 in cell of subject, wherein the method is selected from the group consisting of: neuroprotection, method of treatment of an age-related disease or disorder (excluding cancer), rejuvenation, prevention or amelioration or lessening the effects of aging or other anti-aging treatment.

16. The method of claim 15, wherein disease or condition is selected from the group consisting of:
neurodegenerative disease, neurodegenerative condition, acute central nervous system injury, chronic central nervous system injury, traumatic brain injury; ischemic stroke; reperfusion injury, CLN1 disease, CLN10 disease, CLN2 disease, CLN3 disease, CLN4 disease, CLN6 disease, CLN7 disease, CLN8 disease, Alzheimer's disease, amyotrophic lateral sclerosis, Huntington's disease, Parkinson's disease, myoclonus epilepsy, neuronal damage, Batten disease, progressive supranuclear palsy (Steele-Richardson-Olszewski syndrome), frontotemporal dementia and parkinsonism linked to chromosome 17, basophilic inclusion body disease, Pick disease, dementia with Lewy bodies, frontotemporal lobar degeneration, Lewy body disease, Sandhoff disease; disease or condition, in which treatment increasing of cell antioxidant capacity is beneficial; disease or condition, in which treatment prevention of apoptotic death of neuron is beneficial; disease or condition, in which treatment inhibition reactive astrocyte proliferation is beneficial; disease or condition, in which treatment a protection of neuron against excitotoxicity is beneficial; menopausal syndrome, atherosclerosis, cardiovascular disease, arthritis, cataracts, osteoporosis, type 2 diabetes, hypertension, stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, age related decline in immune response to vaccines, age related decline in response to immunotherapy, myocardial infarction, acute coronary syndrome, sarcopenic obesity, senile osteoporosis, urinary incontinence, neuromuscular disorder, osteoarthritis, chronic fatigue syndrome, Creutzfeldt-Jakob disease, stroke, pre-diabetes, diabetes, obesity, osteoporosis, coronary artery disease, cerebrovascular disease, heart attack, peripheral arterial disease, aortic valve disease, Lewy body disease, progressive subcortical gliosis, progressive supranuclear palsy, thalamic degeneration syndrome, hereditary aphasia, macular degeneration, frailty, pressure ulcers, or any other age related disease, atrophic gastritis, osteoarthritis, sarcopenia, accelerated aging; accelerated aging of cancer survivor; accelerated aging of subject suffering from HIV; consequences of chemotherapy; ataxia telangiectasia (Louis-Bar syndrome), argyrophilic grain disease, autosomal dominant cerebellar ataxia, corticobasal degeneration, corticobasal ganglionic degeneration, Creutzfeldt-Jakob disease, fatal familial insomnia, neuronal intermediate filament inclusion disease, multiple-system atrophy, hereditary motor and sensory neuropathy with proximal dominance, infantile refsum disease, Machado-Joseph disease, mental retardation and microcephaly with pontine and cerebellar hypoplasia (mental retardation, X-linked, syndromic, Najm type), neuroacanthocytosis, pontocerebellar hypoplasia, pyruvate dehydrogenase deficiency (pyruvate dehydrogenase complex deficiency), refsum disease (heredopathia atactica polyneuritiformis), abetalipoproteinemia (Bassen-Kornzweig syndrome), spinal muscular atrophy, Friedreich's ataxia, spinocerebellar ataxia, dentatorubral-pallidoluysian atrophy, Gerstmann-Sträussler-Scheinker syndrome, motor neurone disease, Charcot disease or Lou Gehrig's disease, sclerosis, spinal muscular atrophy, depression, bipolar disorder, Friedreich's ataxia, Spinal muscular atrophy, Motor Neuron Diseases, Alpers' Disease, Cerebro-Oculo-Facio-Skeletal Syndrome (COFS), Corticobasal Degeneration, Gerstmann-Straussler-Scheinker Disease, Kuru, Leigh's Disease, Monomelic Amyotrophy, Multiple System Atrophy, Multiple System Atrophy with Orthostatic Hypotension (Shy-Drager Syndrome), Neurodegeneration with Brain Iron Accumulation, Opsoclonus Myoclonus, Prion Diseases, Progressive Multifocal Leukoencephalopathy, Striatonigral Degeneration, Transmissible Spongiform Encephalopathies (Prion Diseases), Alexander disease, Alpers-Huttenlocher syndrome, alpha-methylacyl-CoA racemase deficiency, Andermann syndrome, Arts syndrome, ataxia neuropathy spectrum, ataxia with oculomotor apraxia, autosomal dominant cerebellar ataxia, deafness, and narcolepsy, autosomal recessive spastic ataxia of Charlevoix-Saguenay, beta-propeller protein-associated neurodegeneration, congenital insensitivity to pain with anhidrosis, familial encephalopathy with neuroserpin inclusion bodies, fatty acid hydroxylase-associated neurodegeneration, GM2-gangliosidosis, AB variant, hereditary sensory and autonomic neuropathy type IE, hereditary sensory and autonomic neuropathy type II, hereditary sensory and autonomic neuropathy type V, infantile neuroaxonal dystrophy, infantile-onset ascending hereditary spastic paralysis, infantile-onset spinocerebellar ataxia, juvenile primary lateral sclerosis, Marinesco-Sjögren syndrome, mitochondrial membrane protein-associated neurodegeneration, multiple system atrophy, neuromyelitis optica, pantothenate kinase-associated neurodegeneration, polycystic lipomembranous osteodysplasia with sclerosing leukoencephalopathy, prion disease, progressive external ophthalmoplegia, riboflavin transporter deficiency neuronopathy, spastic paraplegia type 49, age-progressive dementia, senile dementia, mild cognitive impairment due to aging, mild cognitive impairment, pre-dementia, dementia, CNS cerebral senility, delirium, cognitive decline; or wherein anti-aging treatment is selected from the group consisting of: a method, the method selected from method of treatment or preventing an age-related disease or disorder, a method of maintaining or improving health of a subject, a method of maintaining or improving fitness of a subject, a method of improving/increasing activity in a subject, improving/increasing functional activity in a subject, method of prevention, amelioration or lessening the effects of aging, method of decreasing or delaying an increase in the biological age or slowing rate of aging, method of the changing biomarker or biomarkers of morbidity into the state corresponding to less chances of morbidity, method of treatment, prevention, amelioration and lessening the effects of frailty, method of the treatment of aging related disease, method of the increasing health span or lifespan, method of increasing stress resistance or resilience, method of increasing rate or other enhancement of recovery after surgery,
method of increasing rate or other enhancement of recovery after radiotherapy, disease and/or any other stress, method of the prevention and/or the treatment of menopausal syndrome or restoring reproductive function, method of the eliminating or decrease in spreading of senescent cells, method of the decreasing all-causes or multiple causes of mortality risks or mortality risks related to at least one or at least two of age related diseases or conditions or delaying in increase of such risks, decreasing morbidity risks, method of the modulating at least one of biomarkers of aging into more youthful state or slowing down its change into “elder” state, method of the prevention or treatment of aging related disease.

17. The method of claim 15, comprising administering to the subject an effective amount of the
PFKFB3 inhibitor, wherein PFKFB3 inhibitor is a small molecule.

18. The method of claim 17, wherein small molecule is a compound of Formula (0): 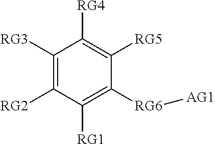
Formula (0) or a pharmaceutically acceptable salt thereof, or N-oxide, or solvate, or tautomer, or stereoisomer, or racemate, including mixtures thereof in all ratios, wherein RG6 and RG5 is one of the following: A) RG6 and RG5 are taken together with the N to which they are attached to form a C₂-C₈ heterocycloalkyl optionally substituted with one or more substituents;
RG1, RG3 and RG4 are independently selected from R_{M}; RG2 is R_{L}; RG5 is Z; RG6 is —C(═O)—; AG1 is —Ar_{C}—Ar_{T};
thus the Formula (0) can be represented as
the Formula (I): 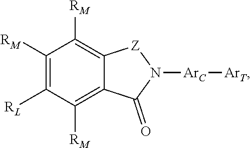
Formula (I), wherein: Z is selected from —C(═O)— and —C(R^{a})(R^{b})—;
R^{a} and R^{b} are independently selected from hydrogen, hydroxyl, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;
wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR¹R², —OH, aryl, heteroaryl, C₁-C₆alkyl, C₁-C₆ alkoxy, C₃-C₈ cycloalkyl, —O—C₃-C₈ cycloalkyl, C₂-C₈ heterocycloalkyl, and -O—C₂-C₈ heterocycloalkyl;
Ar_{C} is selected from C₃-C₈ cycloalkenylene, C₂-C₈ heterocycloalkenylene, arylene, and heteroarylene; wherein Ar_{C} is substituted with one or more R_{C};
each R_{C} are independently selected from —CN, —OH, halogen, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, optionally substituted —O—C₂-C₈ heterocycloalkyl, optionally substituted heteroaryl, optionally substituted aryl, —C(═O)OH, —C(═O)OR³, —C(═O)NR⁴R⁵, —S(═O)₂NR⁴R⁵, —NHC(═O)H, —NHC(═O)R⁶, —NHS(═O)₂R⁶, and —C(═O)NHS(═O)₂R⁶;
wherein the C₁-C₆ alkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, —O—C₃-C₈ cycloalkyl, C₂-C₈ heterocycloalkyl, —O—C₂-C₈ heterocycloalkyl, aryl, and heteroaryl are optionally substituted with one or more substituents independently selected from —OH, halogen, —C(═O)OR⁷, —C(═O)R⁶, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR⁷R⁸;
each R¹ and R² is independently selected from hydrogen, optionally substituted C₁-C₆ alkyl, and optionally substituted C₃-C₅ cycloalkyl;
wherein the C₁-C₆ alkyl is optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl is optionally substituted with one or more substituents independently selected from halogen, —C(═O)OR⁷, —C(═O)NR⁷R⁸, —OH, aryl, heteroaryl, C₁-C₆alkyl, C₁-C₆ alkoxy, C₃-C₈ cycloalkyl, —O—C₃-C₈ cycloalkyl, C₂-C₈ heterocycloalkyl, and -O—C₂-C₈ heterocycloalkyl;
or R¹ and R² are taken together with the N to which they are attached to form a C₂-C₈ heterocycloalkyl optionally substituted with one or more substituents independently selected from halogen, —OH, C₁-C₆ alkyl, and C₁-C₆ alkoxy;
each R³ is independently C₁-C₆ alkyl optionally substituted with one or more substituents independently selected from halogen, —OH, optionally substituted —OC(═O)C₁-C₆alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;
wherein the —OC(═O)C₁-C₆ alkyl and —C(═O)O—C₁-C₆ alkyl are optionally substituted with one or more substituents independently selected from halogen, —OH, and —NR⁷R⁸; and
wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
or each R³ is independently C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;
wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and
wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
each R⁴ and R⁵ is independently selected from hydrogen, optionally substituted C₁-C₆ alkyl, optionally substituted C₃-C₈ cycloalkyl, optionally substituted aryl, and optionally substituted heteroaryl;
wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
or R⁴ and R⁵ are taken together with the N to which they are attached to form a C₂-C₈ heterocycloalkyl optionally substituted with one or more substituents independently selected from halogen, —OH, C₁-C₆ alkyl, and C₁-C₆ alkoxy;
each R⁶ are independently selected from optionally substituted C₁-C₆ alkyl, optionally substituted C₃-C₈ cycloalkyl, optionally substituted aryl, and optionally substituted heteroaryl;
wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
each R⁷ and R⁸ is independently selected from hydrogen and C₁-C₆ alkyl;
or R⁷ and R⁸ are taken together with the N to which they are attached to form a C₂-C₈ heterocycloalkyl optionally substituted with one or more C₁-C₆ alkyl substituents;
Ar_{T} is selected from pyridinyl, pyrimidinyl, pyridazinyl, pyrazinyl, phenyl, pyrrolyl, furanyl, thiophenyl, pyrazolyl, imidazolyl, triazolyl, and tetrazolyl, wherein Ar_{T} is optionally substituted by one or more substituents independently selected from halogen, —OH, —NR⁷R⁸, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;
wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and -O—C₂-C₈ heterocycloalkyl;
each R_{M} is independently selected from hydrogen, halogen, —OH, —CN, optionally substituted C₁-C₆ alkyl, optionally substituted C₁-C₆ alkoxy, optionally substituted C₃-C₈ cycloalkyl, optionally substituted —O—C₃-C₈ cycloalkyl, optionally substituted C₂-C₈ heterocycloalkyl, and optionally substituted —O—C₂-C₈ heterocycloalkyl;
wherein the C₁-C₆ alkyl and C₁-C₆ alkoxy are optionally substituted with one or more substituents independently selected from halogen, —C(═O)OR⁷, —C(═O)NR⁷R⁸, —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, —O—C₃-C₈ cycloalkyl, C₂-C₈ heterocycloalkyl, and —O—C₂-C₈ heterocycloalkyl are optionally substituted with one or more substituents independently selected from halogen, —C(═O)OR⁷, —C(═O)NR⁷R⁸, —OH, C₁-C₆ alkyl, C₁-C₆ alkoxy, aryl, heteroaryl, C₃-C₈ cycloalkyl, —O—C₃-C₈ cycloalkyl, C₂-C₈ heterocycloalkyl, and —O—C₂-C₈ heterocycloalkyl;
R_{L} is selected from —OH, —CN, optionally substituted C₁-C₆ hydroxyalkyl, optionally substituted C₁-C₆ alkoxy, optionally substituted heteroaryl, —C(═O)OH, —C(═O)OR⁹, —C(═O)NR¹⁰R¹¹, —S(═O)₂NR¹⁰R¹¹, —NHC(═O)H, —NHC(═O)R¹², —NHS(═O)₂R¹² and —C(═O)NHS(═O)₂R¹²;
wherein the C₁-C₆ hydroxyalkyl and C₁-C₆ alkoxy are optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the heteroaryl is optionally substituted with one or more of —OH, —O—C(═O)C₁-C₆ alkyl, (C₁-C₄ alkylene)-O—C(═O)C₁-C₆ alkyl, C₁-C₆ alkyl-(aryl), C₁-C₆ alkyl-(heteroaryl), halogen, —C(═O)OR⁷, —C(═O)R¹², —C(═O)NR¹R², C₁-C₆ alkyl, C₁-C₆ hydroxyalkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, optionally substituted aryl, optionally substituted heteroaryl, and —NR¹R²;
R⁹ is C₁-C₆ alkyl optionally substituted with one or more substituents independently selected from halogen, —OH, optionally substituted —OC(═O)C₁-C₆ alkyl, optionally substituted —C(═O)O—C₁-C₆ alkyl, C₁-C₆ alkoxy, —C(═O)OH, —NR¹R², —C(═O)NR¹R², optionally substituted C₂-C₈ heterocycloalkyl, optionally substituted C₃-C₈ cycloalkyl, optionally substituted aryl and optionally substituted heteroaryl;
wherein the —OC(═O)C₁-C₆ alkyl and —C(═O)O—C₁-C₆ alkyl are optionally substituted with one or more substituents independently selected from halogen, —OH, and —NR⁷R⁸; and
wherein the C₂-C₈ heterocycloalkyl, C₃-C₈ cycloalkyl, aryl and heteroaryl are optionally substituted with one or more substituents independently selected from —OH, halogen, —C(═O)OR⁷, —C(═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
or R⁹ is C₃-C₈ cycloalkyl optionally substituted with one or more substituents independently selected from halogen, —OH, optionally substituted C₁-C₆ alkyl, optionally substituted —O(C═O)C₁-C₆ alkyl, optionally substituted —(C═O)OC₁-C₆ alkyl, C₁-C₆ alkoxy, —C(═O)OH, —NR¹R², —(C═O)NR¹R², optionally substituted C₂-C₈ heterocycloalkyl, optionally substituted C₃-C₈ cycloalkyl, optionally substituted aryl and optionally substituted heteroaryl;
wherein the C₁-C₆ alkyl, —OC(═O)C₁-C₆ alkyl and —C(═O)O—C₁-C₆ alkyl are optionally substituted with one or more substituent independently selected from halogen, —OH, and —NR⁷R⁸; and
wherein the C₂-C₈ heterocycloalkyl, C₃-C₈ cycloalkyl, aryl and heteroaryl are optionally substituted with one or more substituents independently selected from —OH, halogen, —C(═O)OR⁷, —(C═O)NR⁷R⁸, C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
each R¹⁰ and R¹¹ is independently selected from hydrogen, optionally substituted C₁-C₆alkyl, optionally substituted C₃-C₈ cycloalkyl, optionally substituted aryl, and optionally substituted heteroaryl;
wherein the C₁-C₆ alkyl is optionally substituted with one or more substituents independently selected from halogen, —C(═O)OR⁷, —C(═O)NR¹R², —OH, aryl (optionally substituted with —OH, halogen, —C(═O)OR⁷, —C(═O)NR⁷R⁸, C₁-C₆ alkyl, C₁-C₆ alkoxy, —NR⁷R⁸), and heteroaryl (optionally substituted with —OH, halogen, —C(═O)OR⁷, —C(═O)NR⁷R⁸, C₁-C₆ alkyl, C₁-C₆ alkoxy, —NR⁷R⁸, C₃-C₈ cycloalkyl, —O—C₃-C₈ cycloalkyl, C₂-C₈ heterocycloalkyl, or —O—C₂-C₈ heterocycloalkyl); and
wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
or R¹⁰ and R¹¹ are taken together with the N to which they are attached to form a C₂-C₈ heterocycloalkyl optionally substituted with one or more substituents independently selected from halogen, —OH, C₁-C₆ alkyl, and C₁-C₆ alkoxy;
R¹² is selected from optionally substituted C₁-C₆ alkyl, optionally substituted C₃-C₈ cycloalkyl, optionally substituted aryl, and optionally substituted heteroaryl;
wherein the C₁-C₆ alkyl is optionally substituted with one or more substituents independently selected from halogen, —C(═O)OR⁷, —C(═O)NR¹R², —OH, aryl, heteroaryl, C₁-C₆ alkoxy, C₃-C₈ cycloalkyl, —O—C₃-C₈ cycloalkyl, C₂-C₈ heterocycloalkyl, and —O—C₂-C₈ heterocycloalkyl; and
wherein the C₃-C₈ cycloalkyl, aryl and heteroaryl are optionally substituted with one or more substituents independently selected from —OH, halogen, —C(═O)OR⁷, —C(═O)NR¹R², C₁-C₆ alkyl, C₁-C₆ alkoxy, C₃-C₈ cycloalkyl, —O—C₃-C₈ cycloalkyl, C₂-C₈ heterocycloalkyl, —O—C₂-C₈ heterocycloalkyl, and —NR⁷R⁸;
provided that:
(i) at least one of R_{C} is not —NHCOR⁶ when R_{L} is —NHCOR¹² and Ar_{C} is heterocycloalkenylene or heteroarylene; or
(j) at least one of R_{C} is not -Me when R_{L} is —OMe; or
(k) at least one of R_{C} is not —OEt when R_{L} is —C(═O)OH; or
(l) at least one of R_{C} is not —OH when R_{L} is —C(═O)OH; or
(m) at least one of R_{C} is not -Me when R_{L} is —C(═O)OH; or
(n) at least one of R_{C} is not -Et when R_{L} is —OMe; or
(o) at least one of R_{C} is not optionally substituted benzoxazolyl when R_{L} is —C(═O)OH; or
(p) at least one of R_{C} is not optionally substituted isoindoline-1,3-dione when R_{L} is —C(═O)OH;
B) RG6 and RG5 do not form a C₂-C₈ heterocycloalkyl; RG1 is R5; RG2 is R1; RG3 is R6; RG4 is R20; RG5 is R4; RG6 is R10; AG1 is A;
thus the Formula (0) can be represented as the Formula (VII): 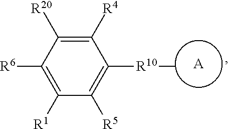
Formula (VII), or a pharmaceutically acceptable salt thereof, wherein:
A is selected from: 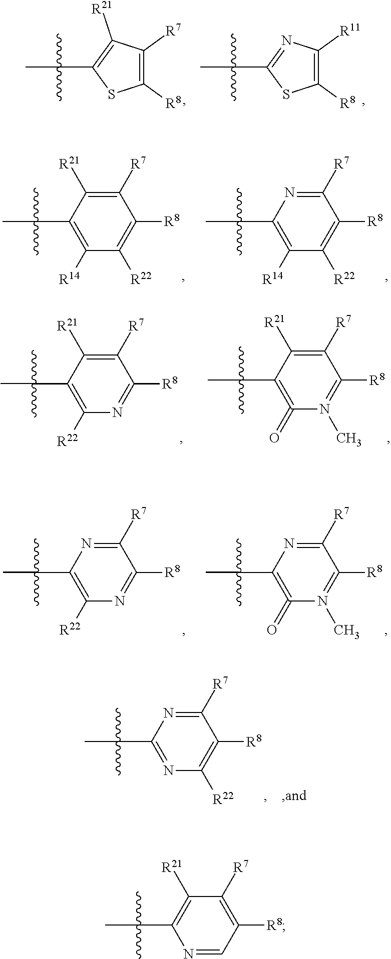
R¹ is selected from hydrogen, halogen, hydroxyl, C₁-C₆ alkyl, and C₁-C₆ alkoxy,
wherein the C₁-C₆ alkyl and C₁-C₆ alkoxy are optionally substituted with one or more halogens;
each R² and R³ is independently selected from hydrogen and C₁-C₆ alkyl,
wherein the C₁-C₆ alkyl is optionally substituted with one or more halogens;
or R² and R³ are taken together with the N to which they are attached to form a 3- to 10-membered heterocycle optionally substituted with one or more substituents independently selected from C₁-C₆ alkyl;
R⁴ is selected from hydrogen, halogen, C₁-C₆ alkyl, and C₁-C₆ alkoxy,
wherein the C₁-C₆ alkyl and C₁-C₆ alkoxy are optionally substituted with one or more substituents independently selected from halogens;
R⁵ is selected from —C(═O)OR¹⁵, —C(═O)NR²R³, —S(═O)₂NR²R³, —C(═O)NHR¹⁵, —CH₂OH, 3-hydroxyoxetan-3-yl, and —NH₂;
R⁶ is selected from hydrogen, halogen, hydroxyl, 5-membered heteroaryl, C₁-C₆ alkyl, —C(═O)OR¹⁵, —C(═O)R¹², —C(═O)NHR¹⁵, and —C(═O)N═S(═X³)(CH₃)₂,
wherein the C₁-C₆ alkyl are optionally substituted with one or more R⁹, and
wherein 5-membered heteroaryl contains at least two heteroatoms and is optionally substituted with one or more substituents independently selected from R¹⁷;
R⁷ is selected from hydrogen, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,
wherein the C₁-C₆ alkyl and C₁-C₆ alkoxy are optionally substituted with one or more halogens, and
wherein C₃-C₈ cycloalkyl, —O—C₃-C₈ cycloalkyl, 3- to 10-membered heterocycloalkyl, —O-(3- to 10-membered heterocycloalkyl), aryl and heteroaryl are optionally substituted with one or more R²⁴;
R⁸ is selected from hydrogen, —NO₂, C₁-C₆ alkyl, aryl, and heteroaryl,
wherein the C₁-C₆ alkyl and C₁-C₆ alkoxy are optionally substituted with one or more substituents independently selected at each occurrence from halogen; and
wherein aryl and heteroaryl are optionally substituted with one or more substituents independently selected at each occurrence from R²³;
or R⁷ and R⁸ are taken together to form a C₅-C₁₀ carbocycle or 5- to 10-membered heterocycle,
wherein C₅-C₁₀ carbocycle and 5- to 10-membered heterocycle are optionally substituted with one or more substituents independently selected from halogen, hydroxyl, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,
wherein the C₁-C₆ alkyl and C₁-C₆ alkoxy are optionally substituted with one or more halogens, and
wherein aryl, heteroaryl, C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;
each R⁹ is independently selected from hydroxy and —COOH;
R¹⁰ is selected from —C(═O)—X¹—, —CH₂—X¹—, —X¹—C(═O)—, and —X¹-CH₂—;
R¹¹ is selected from hydrogen, —NO₂, C₁-C₆ alkyl, C₁-C₆ alkoxy, C₃-C₅ cycloalkyl, —O—C₃-C₈ cycloalkyl, 3- to 10-membered heterocycloalkyl, and —O—(3- to 10-membered heterocycloalkyl),
wherein the C₁-C₆ alkyl and C₁-C₆ alkoxy are optionally substituted with one or more halogens, and
wherein C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;
R¹² is selected from alanine, arginine, asparagine, aspartic acid, asparagine, aspartic acid, cysteine, glutamic acid, glutamine, glutamine, glutamic acid, glycine, histidine, isoleucine, leucine, lysine, methionine, phenylalanine, proline, serine, threonine, tryptophan, tyrosine, and valine, wherein the point of attachment of R¹² is a nitrogen atom;
R¹⁴ is selected from hydrogen, halogen, hydroxyl, nitrile, —C(═O)CR¹⁵ and —C(═O)OR¹⁵;
each R¹⁵ is independently selected from hydrogen and C₁-C₆ alkyl, -heterocyclyl,
wherein the C₁-C₆ alkyl is optionally substituted with one or more substituents independently selected at each occurrence from —C(═O)NR²R³, -heterocyclyl, —NR²R³;
wherein the heterocyclyl is optionally substituted with one or more substituents independently selected at each occurrence from R² and R³
R¹⁷ is selected from C₁-C₆ alkyl, aryl, and 6-membered heteroaryl,
wherein the C₁-C₆ alkyl is optionally substituted with one or more hydroxy, and
wherein aryl and 6-membered heteroaryl are optionally substituted with one or more substituents independently selected from halogen, —R², and —OR²;
R²⁰ is selected from hydrogen, halogen, hydroxyl, —COOH, —NC(═O)R², —OR², 5-membered heteroaryl, C₁-C₆ alkyl, —C(═O)N═S(═X³)(CH₃)₂, —CH₂(OH)CH₂OH and —NH—SO₂—R²,
wherein the 5-membered heteroaryl contains at least two heteroatoms, and
wherein the C₁-C₆ alkyl is optionally substituted with one or more substituents independently selected from 5-membered heteroaryl, wherein the 5-membered heteroaryl contains at least two heteroatoms;
R²¹ is selected from hydrogen and nitrile;
R²² is selected from hydrogen and hydroxy;
each R²³ is independently selected from halogen, C₁-C₆ alkyl, C₁-C₆ alkoxy,
wherein the C₁-C₆ alkyl and C₁-C₆ alkoxy are optionally substituted with one or more substituents independently selected from halogens;
each R²⁴ is independently selected from halogen, C₁-C₆ alkyl, C₁-C₆ alkoxy, 5-membered heteroaryl
wherein the C₁-C₆ alkyl and C₁-C₆ alkoxy are optionally substituted with one or more substituents independently selected from halogens;
each X¹ is independently selected from —NR²— and —CR²R³—; and
each X³ is independently selected from NH and 0.

19. The method of claim 17, wherein small molecule is selected from the group consisting of: 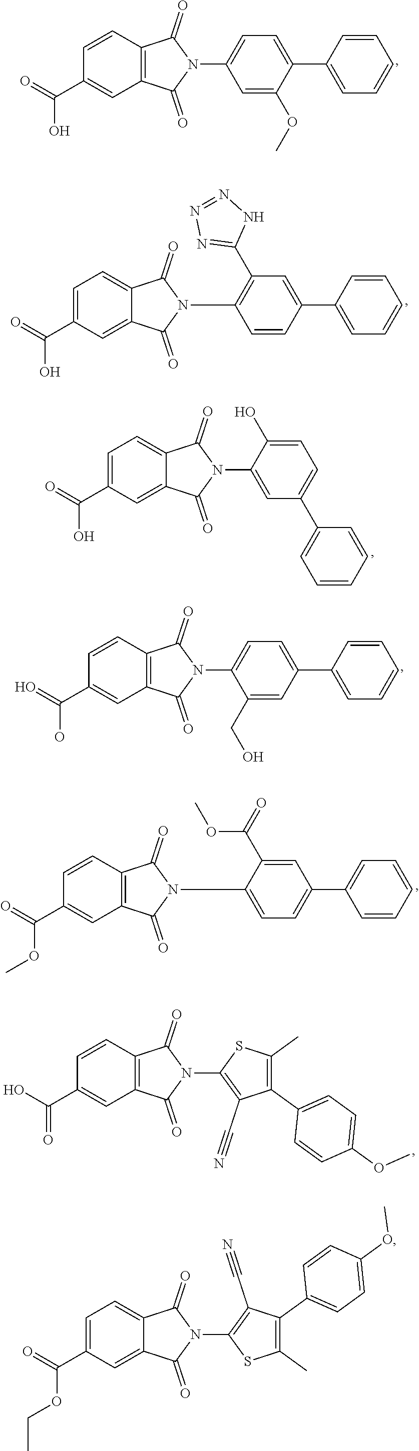 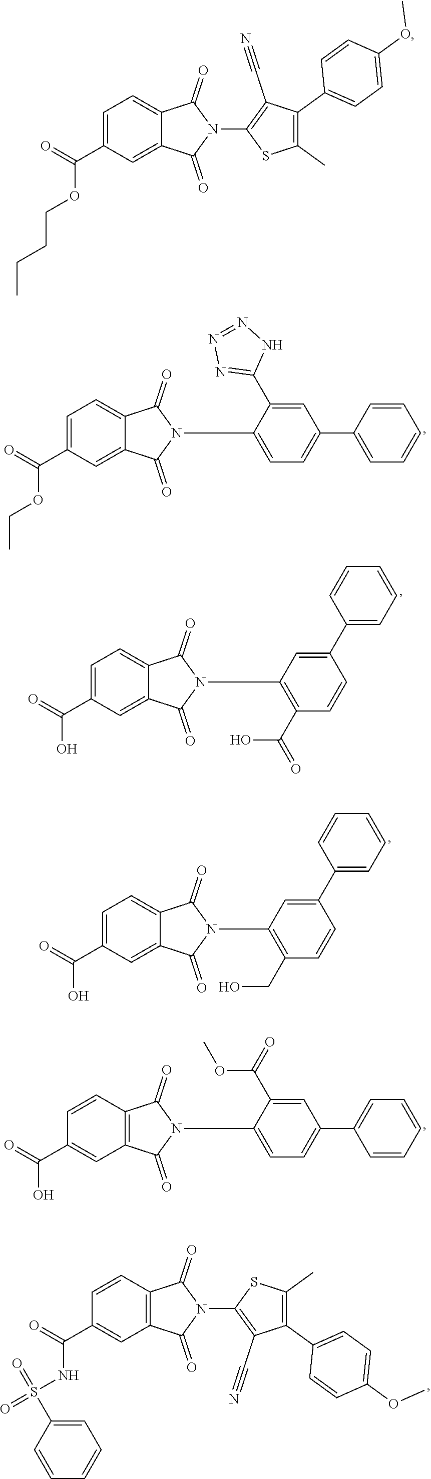 [SMILES: CCCCOC(=O)c1ccc2c(c1)C(=O)N(c1sc(C)c(-c3ccc(OC)cc3)c1C#N)C2=O ; CCOC(=O)c1ccc2c(c1)C(=O)N(c1ccc(-c3ccccc3)cc1-c1nnn[nH]1)C2=O ; O=C(O)c1ccc2c(c1)C(=O)N(c1cc(-c3ccccc3)ccc1C(=O)O)C2=O ; O=C(O)c1ccc2c(c1)C(=O)N(c1cc(-c3ccccc3)ccc1CO)C2=O ; COC(=O)c1cc(-c2ccccc2)ccc1N1C(=O)c2ccc(C(=O)O)cc2C1=O ; COc1ccc(-c2c(C)sc(N3C(=O)c4ccc(C(=O)NS(=O)(=O)c5ccccc5)cc4C3=O)c2C#N)cc1] 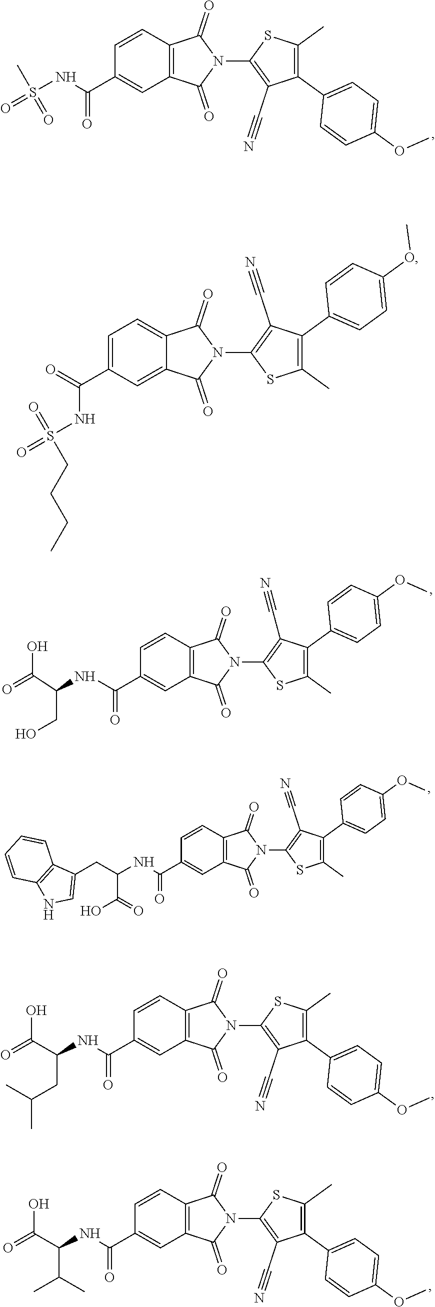 [SMILES: COc1ccc(-c2c(C)sc(N3C(=O)c4ccc(C(=O)NS(C)(=O)=O)cc4C3=O)c2C#N)cc1 ; CCCCS(=O)(=O)NC(=O)c1ccc2c(c1)C(=O)N(c1sc(C)c(-c3ccc(OC)cc3)c1C#N)C2=O ; COc1ccc(-c2c(C)sc(N3C(=O)c4ccc(C(=O)N[C@@H](CO)C(=O)O)cc4C3=O)c2C#N)cc1 ; COc1ccc(-c2c(C)sc(N3C(=O)c4ccc(C(=O)NC(Cc5c[nH]c6ccccc56)C(=O)O)cc4C3=O)c2C#N)cc1 ; COc1ccc(-c2c(C)sc(N3C(=O)c4ccc(C(=O)N[C@@H](CC(C)C)C(=O)O)cc4C3=O)c2C#N)cc1 ; COc1ccc(-c2c(C)sc(N3C(=O)c4ccc(C(=O)N[C@H](C(=O)O)C(C)C)cc4C3=O)c2C#N)cc1] 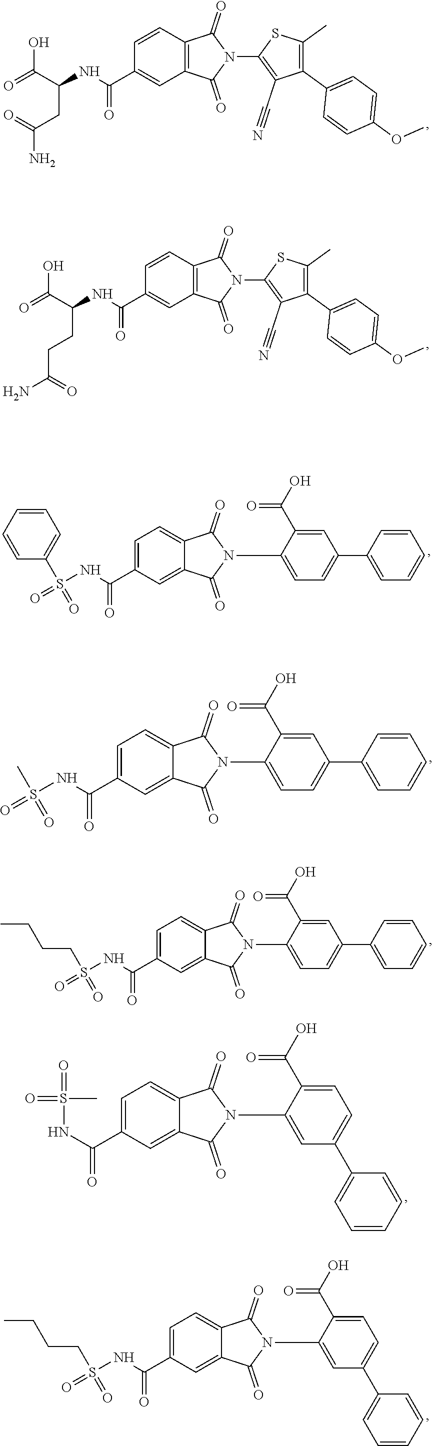 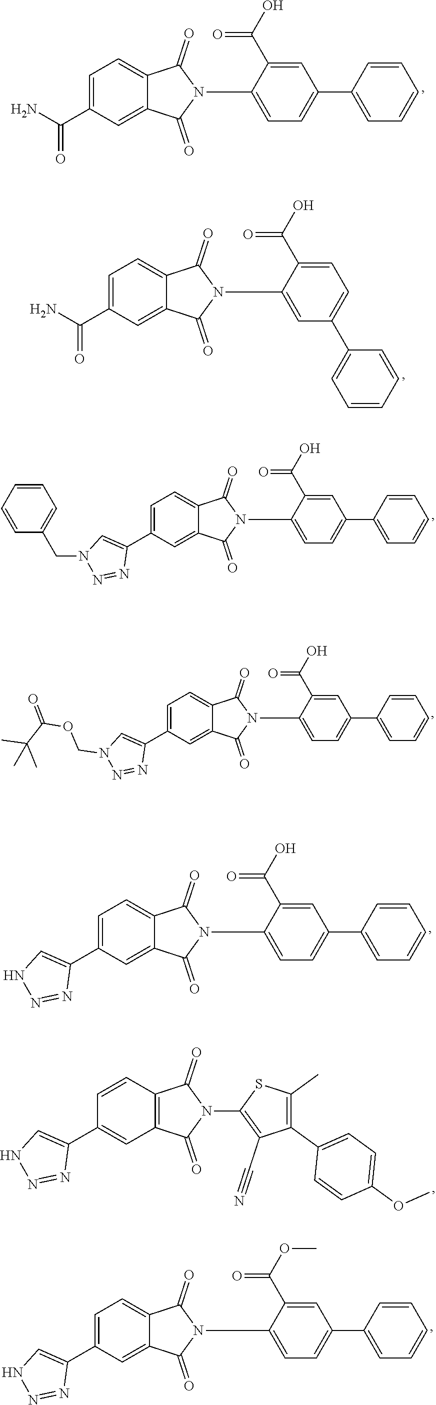 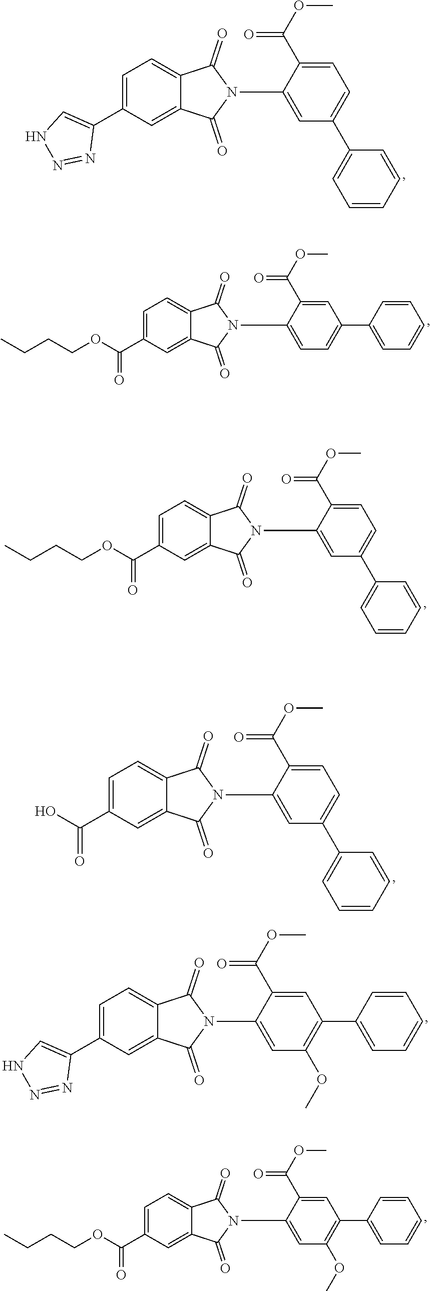 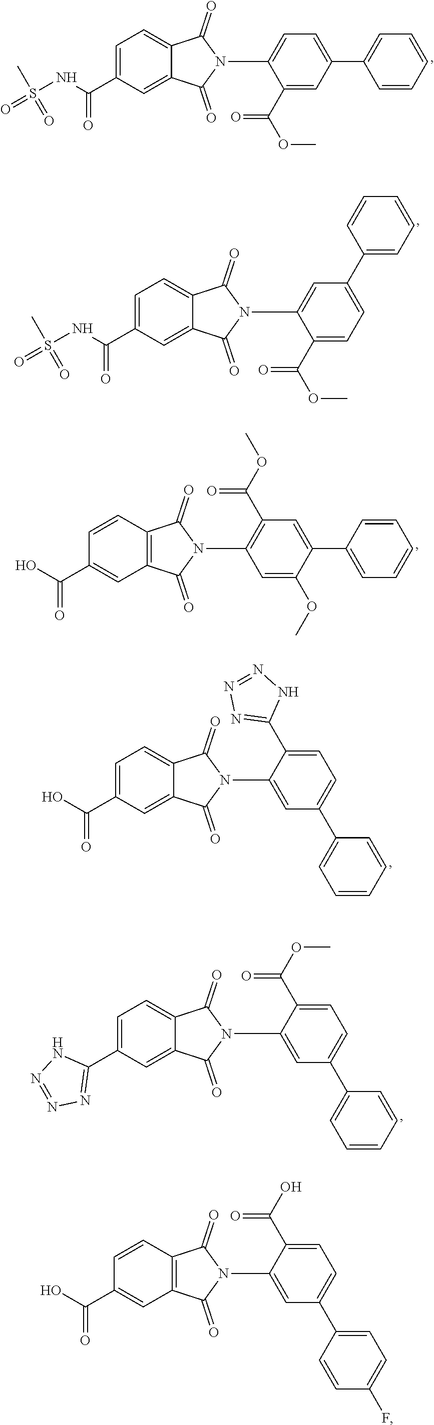 [SMILES: COC(=O)c1cc(-c2ccccc2)ccc1N1C(=O)c2ccc(C(=O)NS(C)(=O)=O)cc2C1=O ; COC(=O)c1ccc(-c2ccccc2)cc1N1C(=O)c2ccc(C(=O)NS(C)(=O)=O)cc2C1=O ; COC(=O)c1cc(-c2ccccc2)c(OC)cc1N1C(=O)c2ccc(C(=O)O)cc2C1=O ; O=C(O)c1ccc2c(c1)C(=O)N(c1cc(-c3ccccc3)ccc1-c1nnn[nH]1)C2=O ; COC(=O)c1ccc(-c2ccccc2)cc1N1C(=O)c2ccc(-c3nnn[nH]3)cc2C1=O ; O=C(O)c1ccc2c(c1)C(=O)N(c1cc(-c3ccc(F)cc3)ccc1C(=O)O)C2=O] 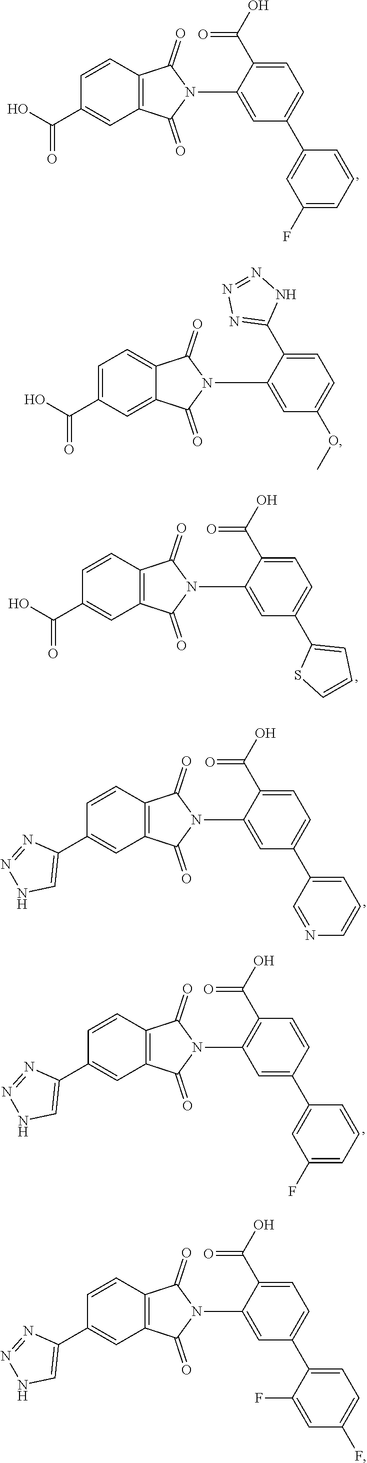 [SMILES: O=C(O)c1ccc2c(c1)C(=O)N(c1cc(-c3cccc(F)c3)ccc1C(=O)O)C2=O ; COc1ccc(-c2nnn[nH]2)c(N2C(=O)c3ccc(C(=O)O)cc3C2=O)c1 ; O=C(O)c1ccc2c(c1)C(=O)N(c1cc(-c3cccs3)ccc1C(=O)O)C2=O ; O=C(O)c1ccc(-c2cccnc2)cc1N1C(=O)c2ccc(-c3c[nH]nn3)cc2C1=O ; O=C(O)c1ccc(-c2cccc(F)c2)cc1N1C(=O)c2ccc(-c3c[nH]nn3)cc2C1=O ; O=C(O)c1ccc(-c2ccc(F)cc2F)cc1N1C(=O)c2ccc(-c3c[nH]nn3)cc2C1=O] 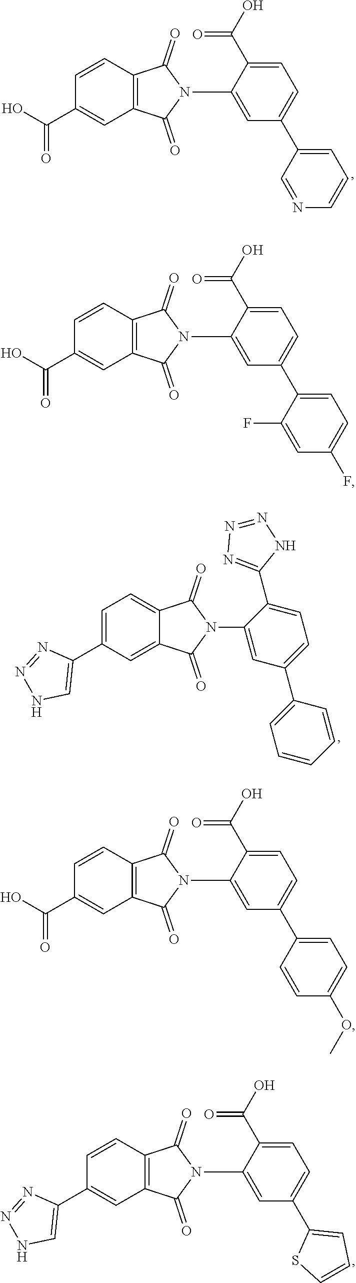 [SMILES: O=C(O)c1ccc2c(c1)C(=O)N(c1cc(-c3cccnc3)ccc1C(=O)O)C2=O ; O=C(O)c1ccc2c(c1)C(=O)N(c1cc(-c3ccc(F)cc3F)ccc1C(=O)O)C2=O ; O=C1c2ccc(-c3c[nH]nn3)cc2C(=O)N1c1cc(-c2ccccc2)ccc1-c1nnn[nH]1 ; COc1ccc(-c2ccc(C(=O)O)c(N3C(=O)c4ccc(C(=O)O)cc4C3=O)c2)cc1 ; O=C(O)c1ccc(-c2cccs2)cc1N1C(=O)c2ccc(-c3c[nH]nn3)cc2C1=O] 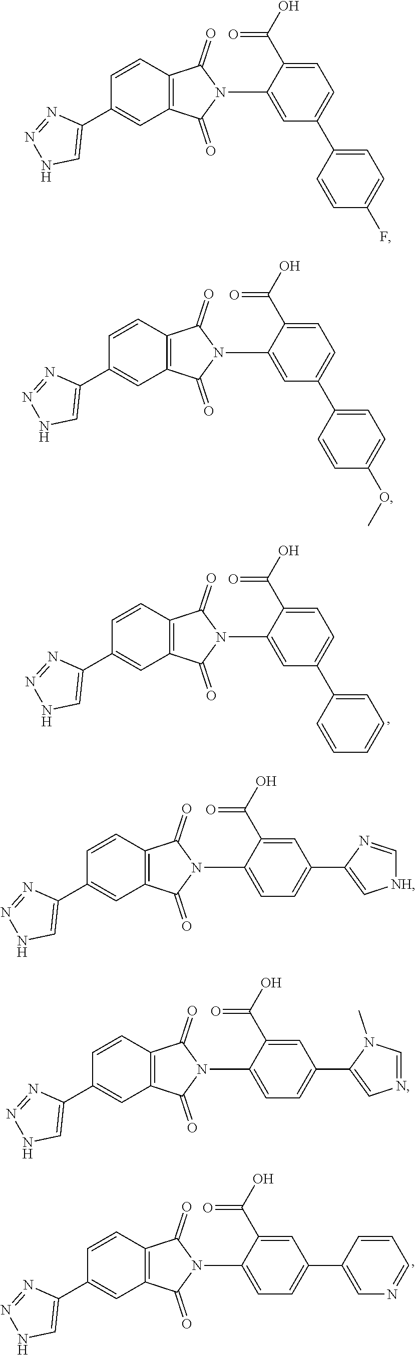 [SMILES: O=C(O)c1ccc(-c2ccc(F)cc2)cc1N1C(=O)c2ccc(-c3c[nH]nn3)cc2C1=O ; COc1ccc(-c2ccc(C(=O)O)c(N3C(=O)c4ccc(-c5c[nH]nn5)cc4C3=O)c2)cc1 ; O=C(O)c1ccc(-c2ccccc2)cc1N1C(=O)c2ccc(-c3c[nH]nn3)cc2C1=O ; O=C(O)c1cc(-c2c[nH]cn2)ccc1N1C(=O)c2ccc(-c3c[nH]nn3)cc2C1=O ; Cn1cncc1-c1ccc(N2C(=O)c3ccc(-c4c[nH]nn4)cc3C2=O)c(C(=O)O)c1 ; O=C(O)c1cc(-c2cccnc2)ccc1N1C(=O)c2ccc(-c3c[nH]nn3)cc2C1=O] 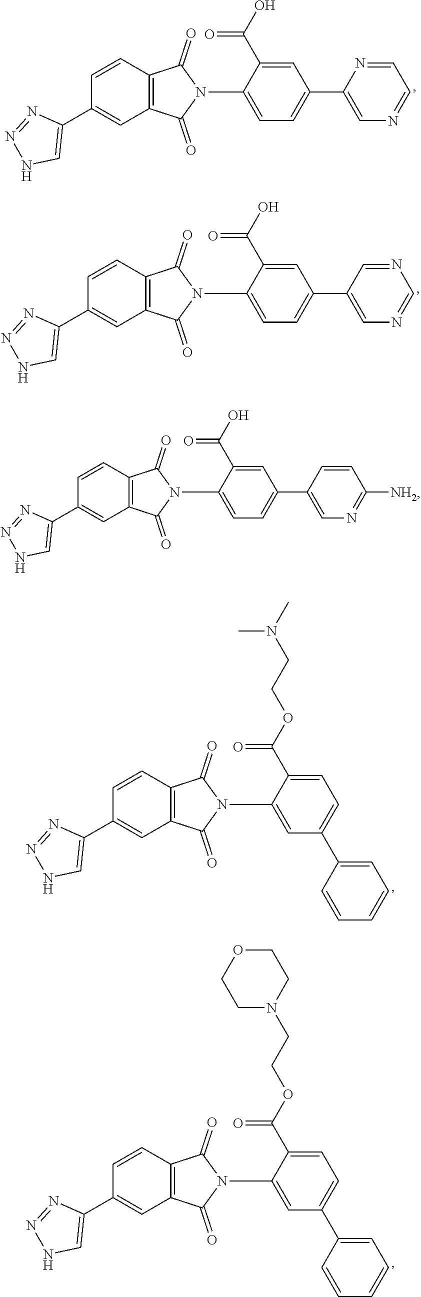 [SMILES: O=C(O)c1cc(-c2cnccn2)ccc1N1C(=O)c2ccc(-c3c[nH]nn3)cc2C1=O ; O=C(O)c1cc(-c2cncnc2)ccc1N1C(=O)c2ccc(-c3c[nH]nn3)cc2C1=O ; Nc1ccc(-c2ccc(N3C(=O)c4ccc(-c5c[nH]nn5)cc4C3=O)c(C(=O)O)c2)cn1 ; CN(C)CCOC(=O)c1ccc(-c2ccccc2)cc1N1C(=O)c2ccc(-c3c[nH]nn3)cc2C1=O ; O=C(OCCN1CCOCC1)c1ccc(-c2ccccc2)cc1N1C(=O)c2ccc(-c3c[nH]nn3)cc2C1=O] 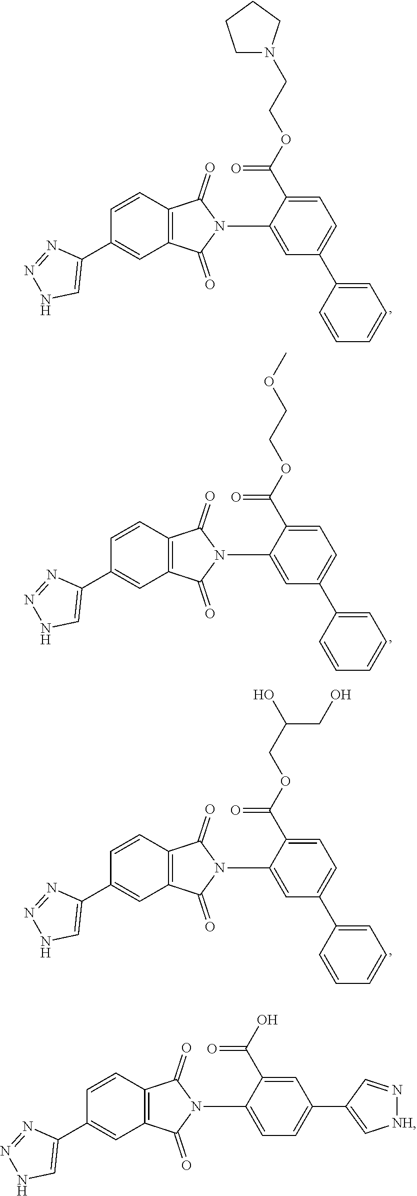 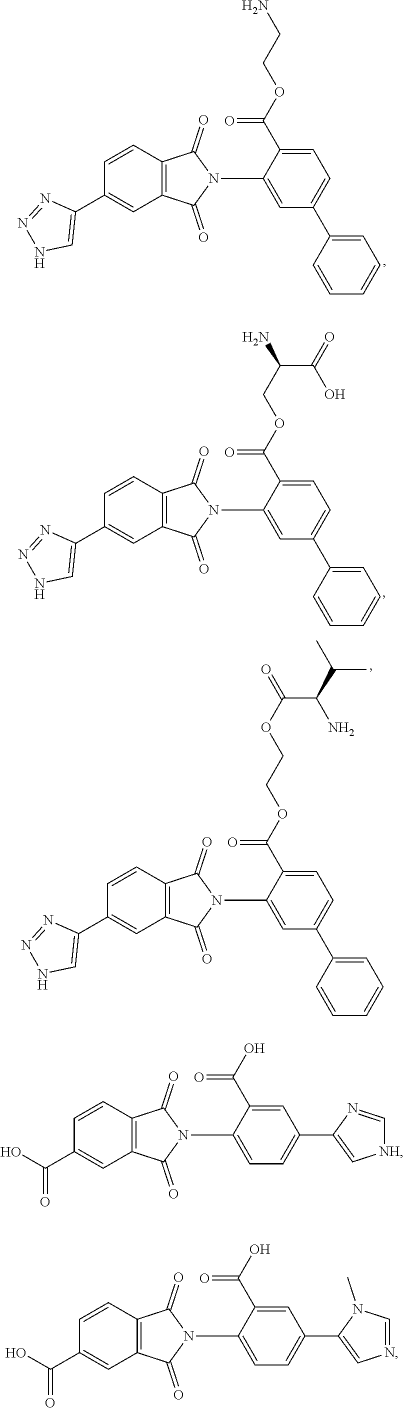 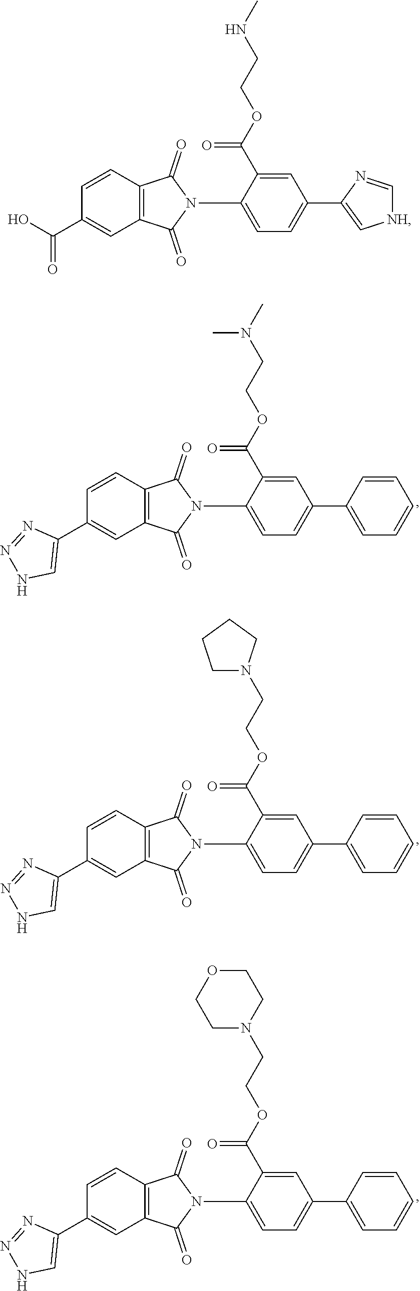 [SMILES: CNCCOC(=O)c1cc(-c2c[nH]cn2)ccc1N1C(=O)c2ccc(C(=O)O)cc2C1=O ; CN(C)CCOC(=O)c1cc(-c2ccccc2)ccc1N1C(=O)c2ccc(-c3c[nH]nn3)cc2C1=O ; O=C(OCCN1CCCC1)c1cc(-c2ccccc2)ccc1N1C(=O)c2ccc(-c3c[nH]nn3)cc2C1=O ; O=C(OCCN1CCOCC1)c1cc(-c2ccccc2)ccc1N1C(=O)c2ccc(-c3c[nH]nn3)cc2C1=O] 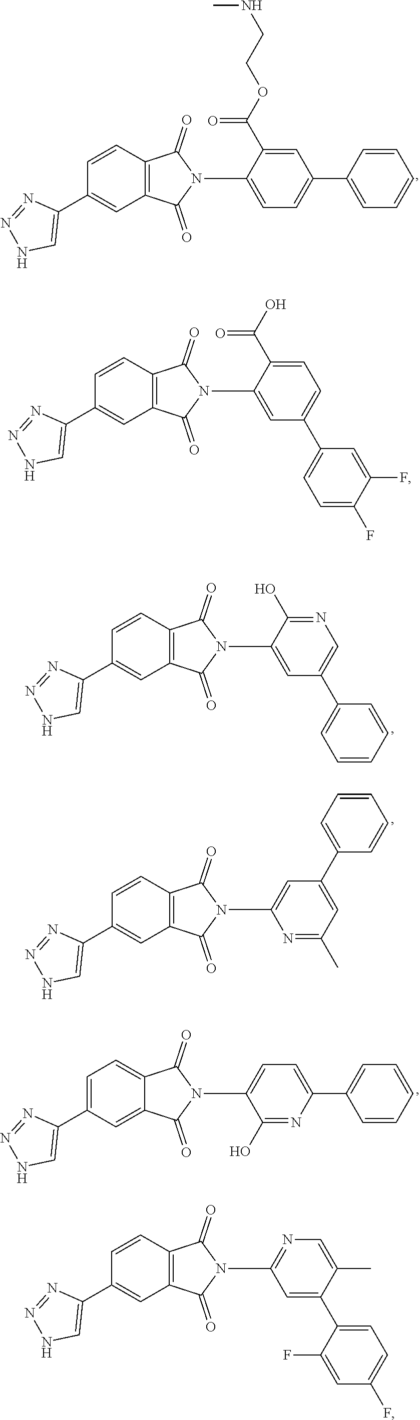 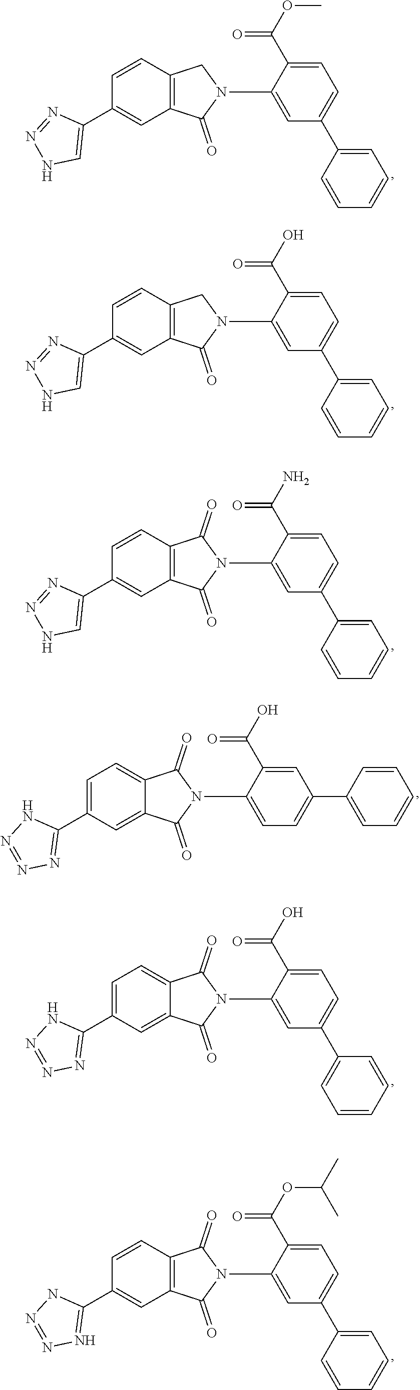 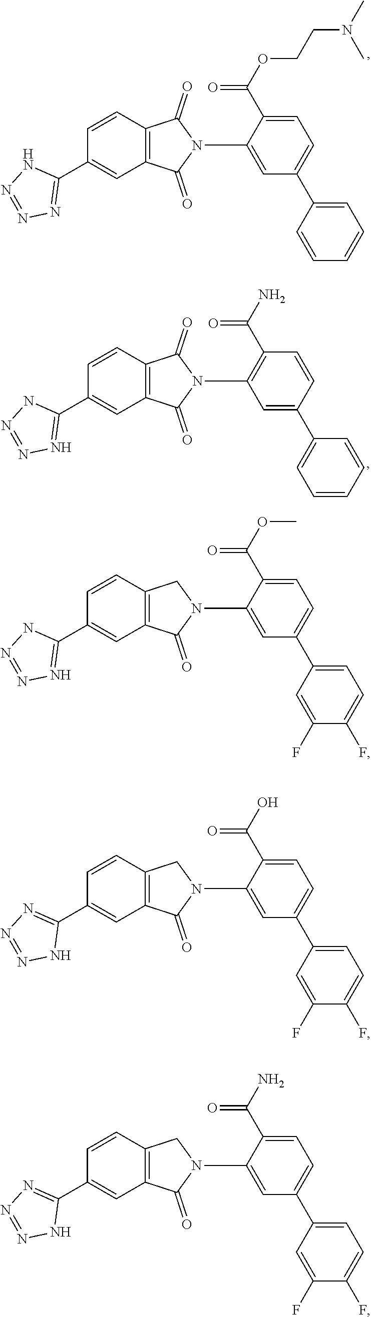 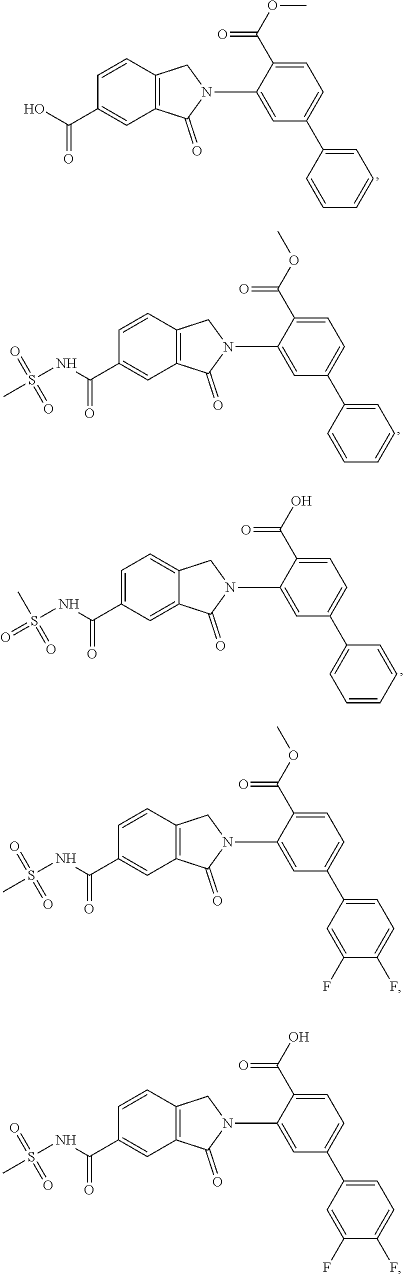 [SMILES: COC(=O)c1ccc(-c2ccccc2)cc1N1Cc2ccc(C(=O)O)cc2C1=O ; COC(=O)c1ccc(-c2ccccc2)cc1N1Cc2ccc(C(=O)NS(C)(=O)=O)cc2C1=O ; CS(=O)(=O)NC(=O)c1ccc2c(c1)C(=O)N(c1cc(-c3ccccc3)ccc1C(=O)O)C2 ; COC(=O)c1ccc(-c2ccc(F)c(F)c2)cc1N1Cc2ccc(C(=O)NS(C)(=O)=O)cc2C1=O ; CS(=O)(=O)NC(=O)c1ccc2c(c1)C(=O)N(c1cc(-c3ccc(F)c(F)c3)ccc1C(=O)O)C2] 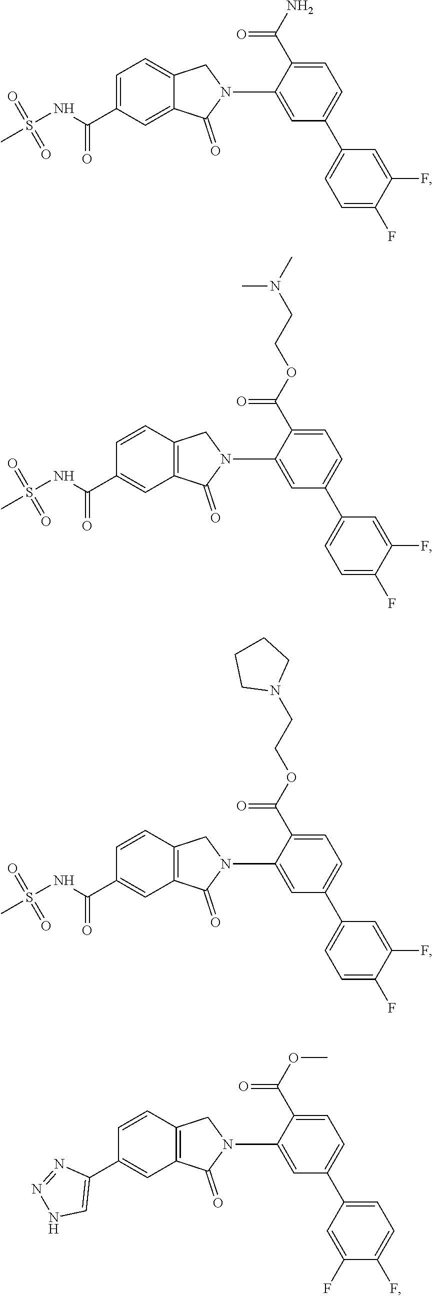 [SMILES: CS(=O)(=O)NC(=O)c1ccc2c(c1)C(=O)N(c1cc(-c3ccc(F)c(F)c3)ccc1C(N)=O)C2 ; CN(C)CCOC(=O)c1ccc(-c2ccc(F)c(F)c2)cc1N1Cc2ccc(C(=O)NS(C)(=O)=O)cc2C1=O ; CS(=O)(=O)NC(=O)c1ccc2c(c1)C(=O)N(c1cc(-c3ccc(F)c(F)c3)ccc1C(=O)OCCN1CCCC1)C2 ; COC(=O)c1ccc(-c2ccc(F)c(F)c2)cc1N1Cc2ccc(-c3c[nH]nn3)cc2C1=O] 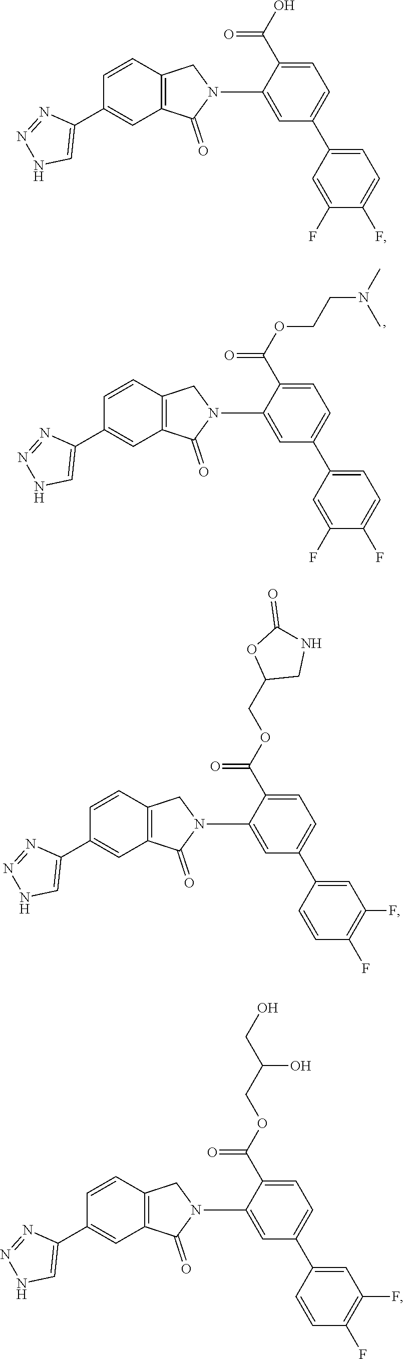 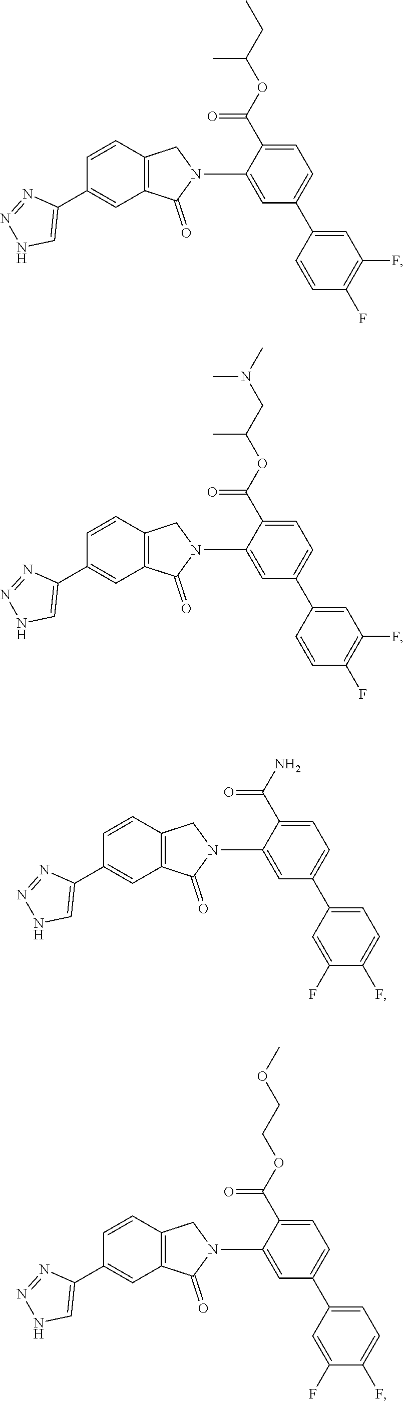 [SMILES: CCC(C)OC(=O)c1ccc(-c2ccc(F)c(F)c2)cc1N1Cc2ccc(-c3c[nH]nn3)cc2C1=O ; CC(CN(C)C)OC(=O)c1ccc(-c2ccc(F)c(F)c2)cc1N1Cc2ccc(-c3c[nH]nn3)cc2C1=O ; NC(=O)c1ccc(-c2ccc(F)c(F)c2)cc1N1Cc2ccc(-c3c[nH]nn3)cc2C1=O ; COCCOC(=O)c1ccc(-c2ccc(F)c(F)c2)cc1N1Cc2ccc(-c3c[nH]nn3)cc2C1=O] 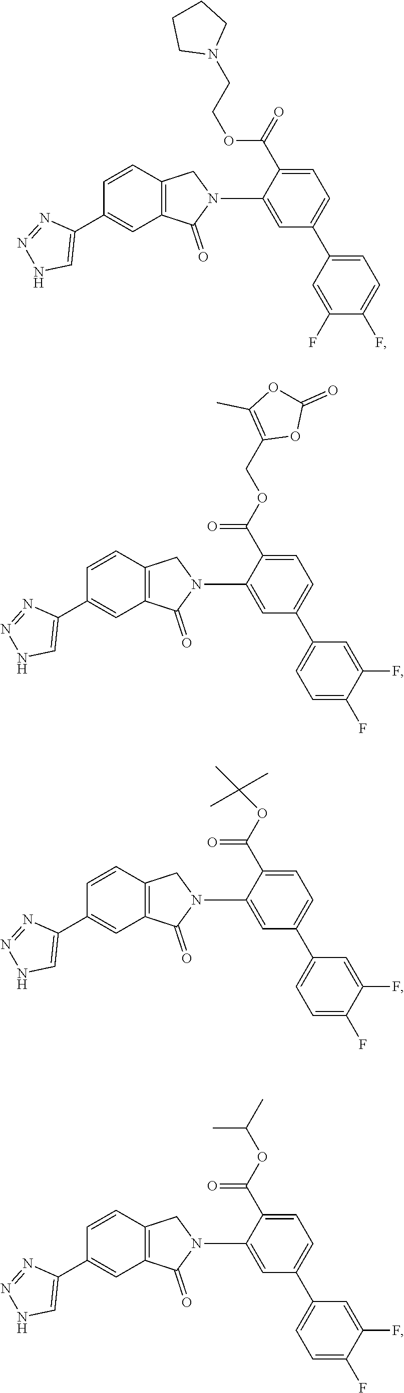 [SMILES: O=C(OCCN1CCCC1)c1ccc(-c2ccc(F)c(F)c2)cc1N1Cc2ccc(-c3c[nH]nn3)cc2C1=O ; Cc1oc(=O)oc1COC(=O)c1ccc(-c2ccc(F)c(F)c2)cc1N1Cc2ccc(-c3c[nH]nn3)cc2C1=O ; CC(C)(C)OC(=O)c1ccc(-c2ccc(F)c(F)c2)cc1N1Cc2ccc(-c3c[nH]nn3)cc2C1=O ; CC(C)OC(=O)c1ccc(-c2ccc(F)c(F)c2)cc1N1Cc2ccc(-c3c[nH]nn3)cc2C1=O] 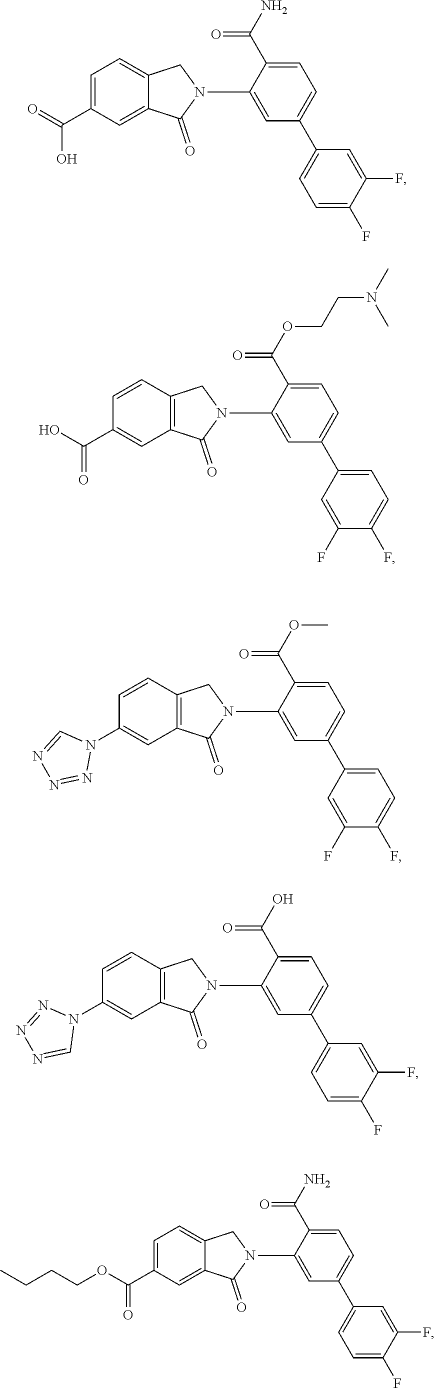 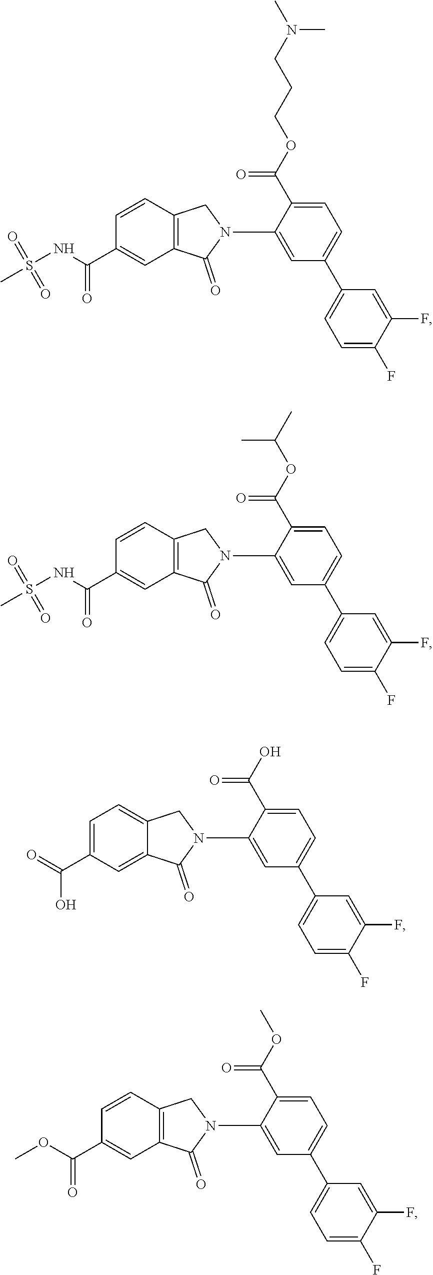 [SMILES: CN(C)CCCOC(=O)c1ccc(-c2ccc(F)c(F)c2)cc1N1Cc2ccc(C(=O)NS(C)(=O)=O)cc2C1=O ; CC(C)OC(=O)c1ccc(-c2ccc(F)c(F)c2)cc1N1Cc2ccc(C(=O)NS(C)(=O)=O)cc2C1=O ; O=C(O)c1ccc2c(c1)C(=O)N(c1cc(-c3ccc(F)c(F)c3)ccc1C(=O)O)C2 ; COC(=O)c1ccc2c(c1)C(=O)N(c1cc(-c3ccc(F)c(F)c3)ccc1C(=O)OC)C2] 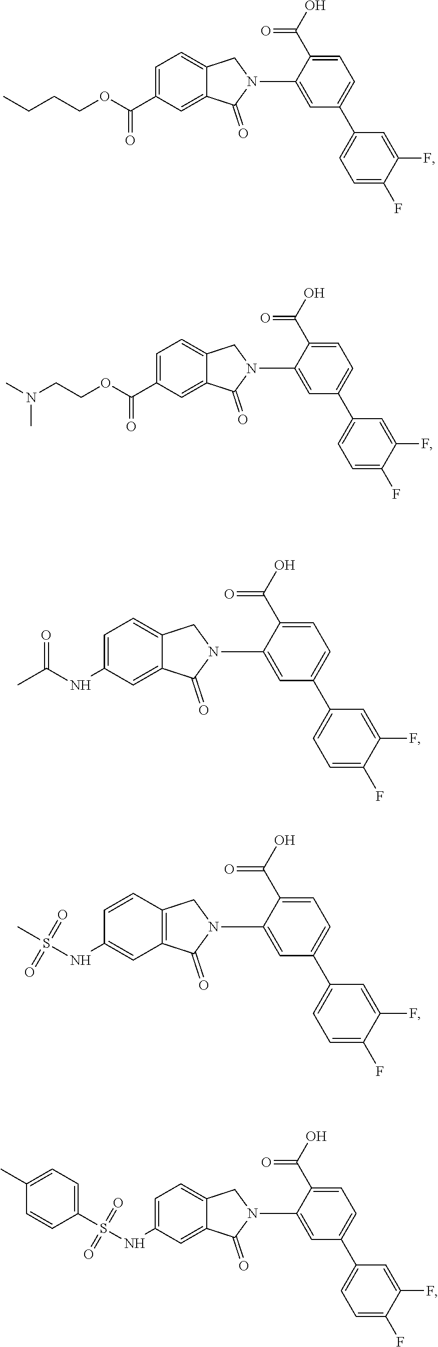 [SMILES: CCCCOC(=O)c1ccc2c(c1)C(=O)N(c1cc(-c3ccc(F)c(F)c3)ccc1C(=O)O)C2 ; CN(C)CCOC(=O)c1ccc2c(c1)C(=O)N(c1cc(-c3ccc(F)c(F)c3)ccc1C(=O)O)C2 ; CC(=O)Nc1ccc2c(c1)C(=O)N(c1cc(-c3ccc(F)c(F)c3)ccc1C(=O)O)C2 ; CS(=O)(=O)Nc1ccc2c(c1)C(=O)N(c1cc(-c3ccc(F)c(F)c3)ccc1C(=O)O)C2 ; Cc1ccc(S(=O)(=O)Nc2ccc3c(c2)C(=O)N(c2cc(-c4ccc(F)c(F)c4)ccc2C(=O)O)C3)cc1] 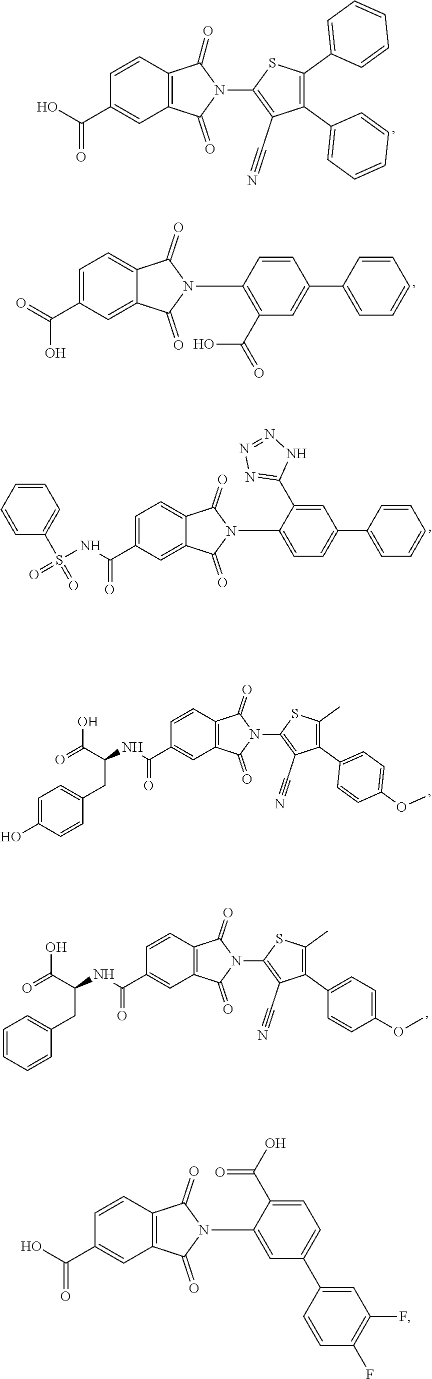 [SMILES: N#Cc1c(N2C(=O)c3ccc(C(=O)O)cc3C2=O)sc(-c2ccccc2)c1-c1ccccc1 ; O=C(O)c1ccc2c(c1)C(=O)N(c1ccc(-c3ccccc3)cc1C(=O)O)C2=O ; O=C(NS(=O)(=O)c1ccccc1)c1ccc2c(c1)C(=O)N(c1ccc(-c3ccccc3)cc1-c1nnn[nH]1)C2=O ; COc1ccc(-c2c(C)sc(N3C(=O)c4ccc(C(=O)N[C@@H](Cc5ccc(O)cc5)C(=O)O)cc4C3=O)c2C#N)cc1 ; COc1ccc(-c2c(C)sc(N3C(=O)c4ccc(C(=O)N[C@@H](Cc5ccccc5)C(=O)O)cc4C3=O)c2C#N)cc1 ; O=C(O)c1ccc2c(c1)C(=O)N(c1cc(-c3ccc(F)c(F)c3)ccc1C(=O)O)C2=O] 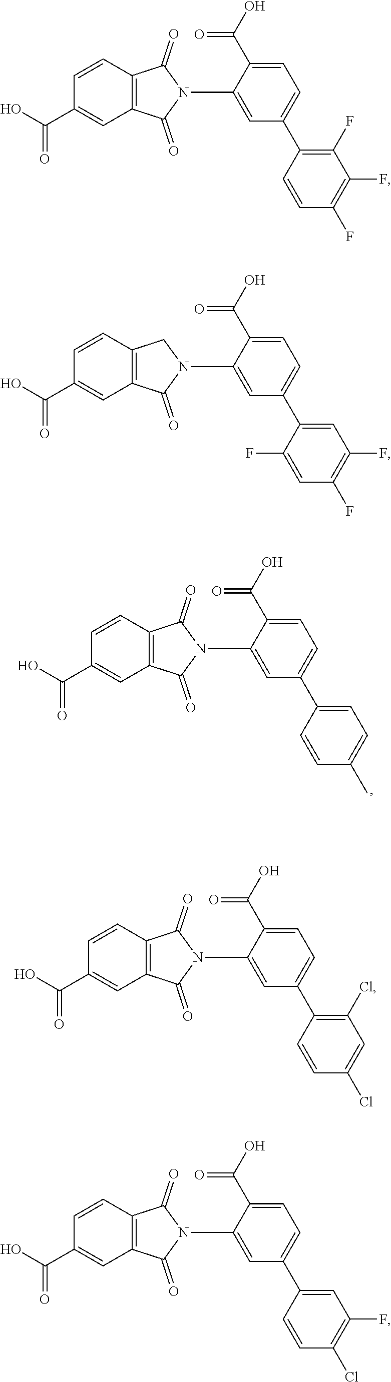 [SMILES: O=C(O)c1ccc2c(c1)C(=O)N(c1cc(-c3ccc(F)c(F)c3F)ccc1C(=O)O)C2=O ; O=C(O)c1ccc2c(c1)C(=O)N(c1cc(-c3cc(F)c(F)cc3F)ccc1C(=O)O)C2 ; Cc1ccc(-c2ccc(C(=O)O)c(N3C(=O)c4ccc(C(=O)O)cc4C3=O)c2)cc1 ; O=C(O)c1ccc2c(c1)C(=O)N(c1cc(-c3ccc(Cl)cc3Cl)ccc1C(=O)O)C2=O ; O=C(O)c1ccc2c(c1)C(=O)N(c1cc(-c3ccc(Cl)c(F)c3)ccc1C(=O)O)C2=O] 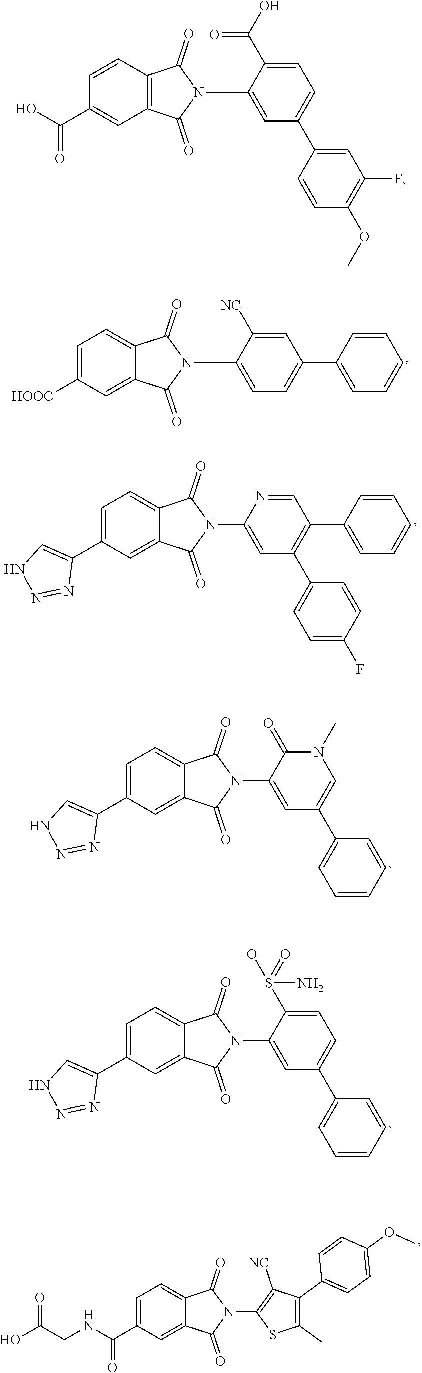 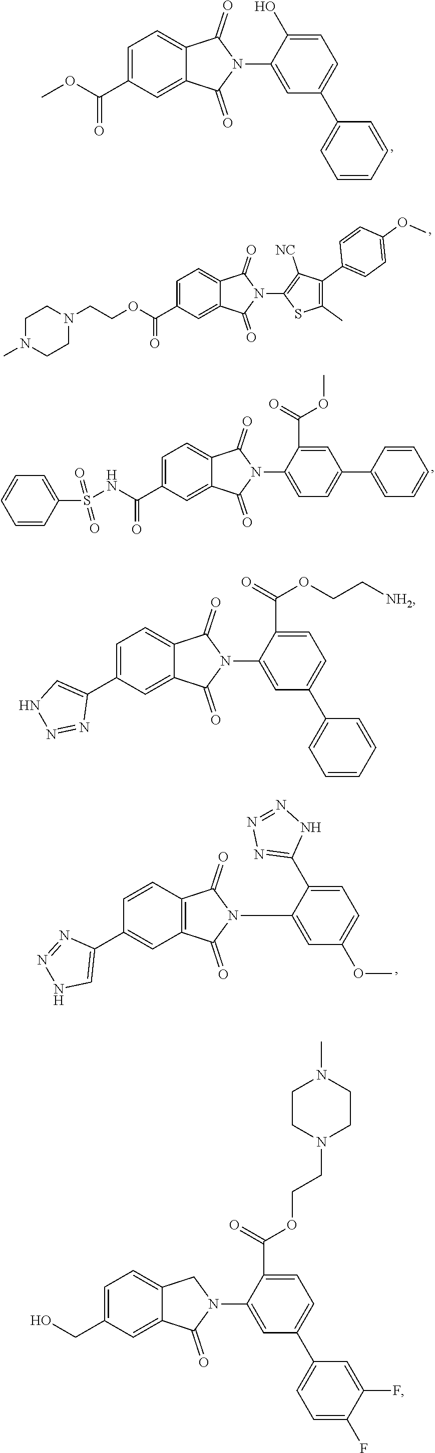 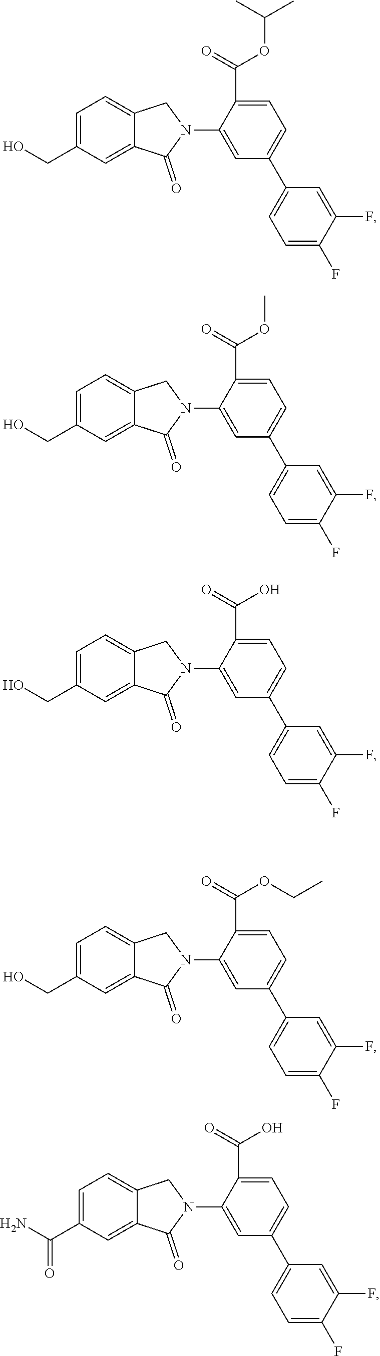 [SMILES: CC(C)OC(=O)c1ccc(-c2ccc(F)c(F)c2)cc1N1Cc2ccc(CO)cc2C1=O ; COC(=O)c1ccc(-c2ccc(F)c(F)c2)cc1N1Cc2ccc(CO)cc2C1=O ; O=C(O)c1ccc(-c2ccc(F)c(F)c2)cc1N1Cc2ccc(CO)cc2C1=O ; CCOC(=O)c1ccc(-c2ccc(F)c(F)c2)cc1N1Cc2ccc(CO)cc2C1=O ; NC(=O)c1ccc2c(c1)C(=O)N(c1cc(-c3ccc(F)c(F)c3)ccc1C(=O)O)C2] 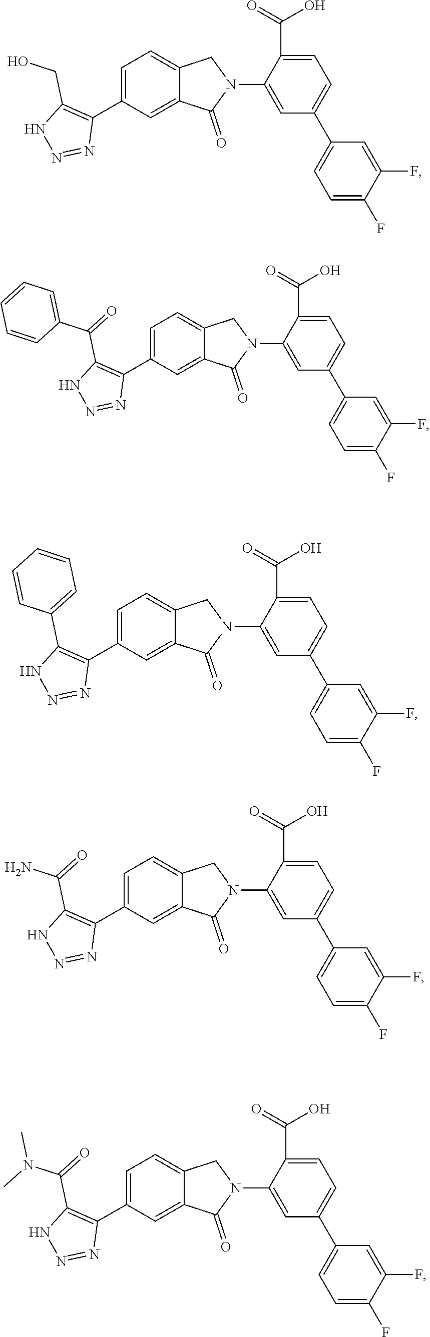 [SMILES: O=C(O)c1ccc(-c2ccc(F)c(F)c2)cc1N1Cc2ccc(-c3nn[nH]c3CO)cc2C1=O ; O=C(O)c1ccc(-c2ccc(F)c(F)c2)cc1N1Cc2ccc(-c3nn[nH]c3C(=O)c3ccccc3)cc2C1=O ; O=C(O)c1ccc(-c2ccc(F)c(F)c2)cc1N1Cc2ccc(-c3nn[nH]c3-c3ccccc3)cc2C1=O ; NC(=O)c1[nH]nnc1-c1ccc2c(c1)C(=O)N(c1cc(-c3ccc(F)c(F)c3)ccc1C(=O)O)C2 ; CN(C)C(=O)c1[nH]nnc1-c1ccc2c(c1)C(=O)N(c1cc(-c3ccc(F)c(F)c3)ccc1C(=O)O)C2] 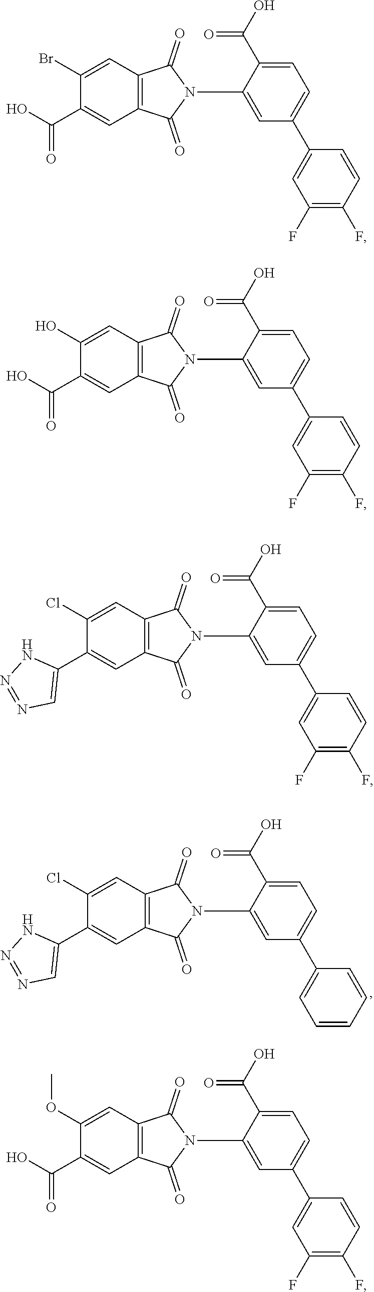 [SMILES: O=C(O)c1cc2c(cc1Br)C(=O)N(c1cc(-c3ccc(F)c(F)c3)ccc1C(=O)O)C2=O ; O=C(O)c1cc2c(cc1O)C(=O)N(c1cc(-c3ccc(F)c(F)c3)ccc1C(=O)O)C2=O ; O=C(O)c1ccc(-c2ccc(F)c(F)c2)cc1N1C(=O)c2cc(Cl)c(-c3cnn[nH]3)cc2C1=O ; O=C(O)c1ccc(-c2ccccc2)cc1N1C(=O)c2cc(Cl)c(-c3cnn[nH]3)cc2C1=O ; COc1cc2c(cc1C(=O)O)C(=O)N(c1cc(-c3ccc(F)c(F)c3)ccc1C(=O)O)C2=O] 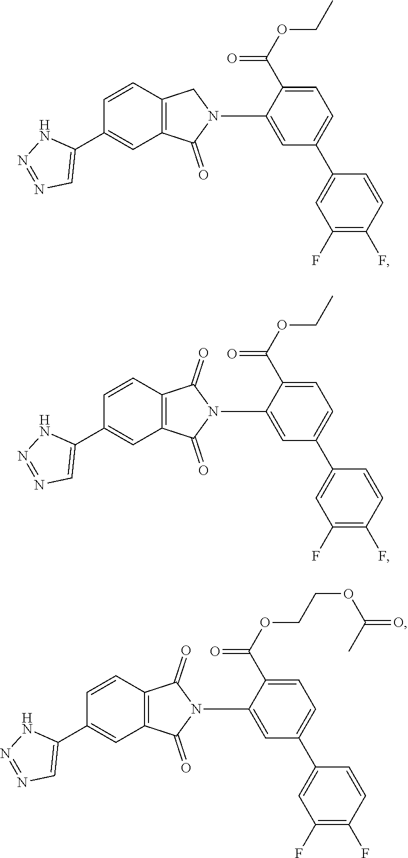
or a pharmaceutically acceptable salt thereof.

20. The method of claim 17, wherein small molecule is selected from the group consisting of: 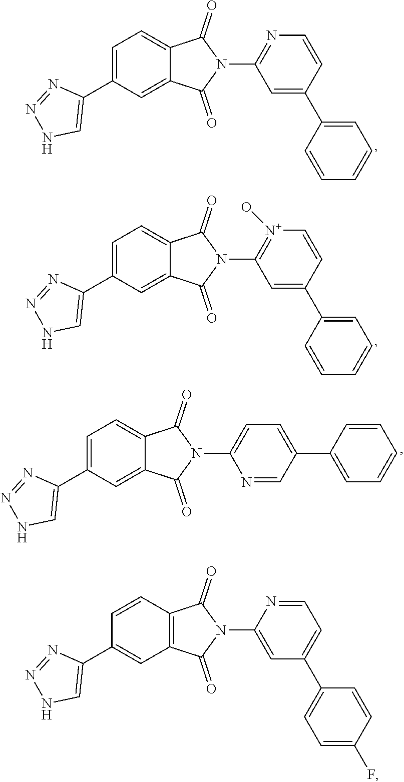 [SMILES: O=C1c2ccc(-c3c[nH]nn3)cc2C(=O)N1c1cc(-c2ccccc2)ccn1 ; O=C1c2ccc(-c3c[nH]nn3)cc2C(=O)N1c1cc(-c2ccccc2)cc[n+]1O ; O=C1c2ccc(-c3c[nH]nn3)cc2C(=O)N1c1ccc(-c2ccccc2)cn1 ; O=C1c2ccc(-c3c[nH]nn3)cc2C(=O)N1c1cc(-c2ccc(F)cc2)ccn1]
or a pharmaceutically acceptable salt thereof.

21. The method of claim 18, wherein the small molecule of Formula (I) is represented by Formula Formula (VII): 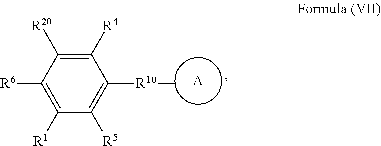
or a pharmaceutically acceptable salt thereof,
wherein:
A is selected from: 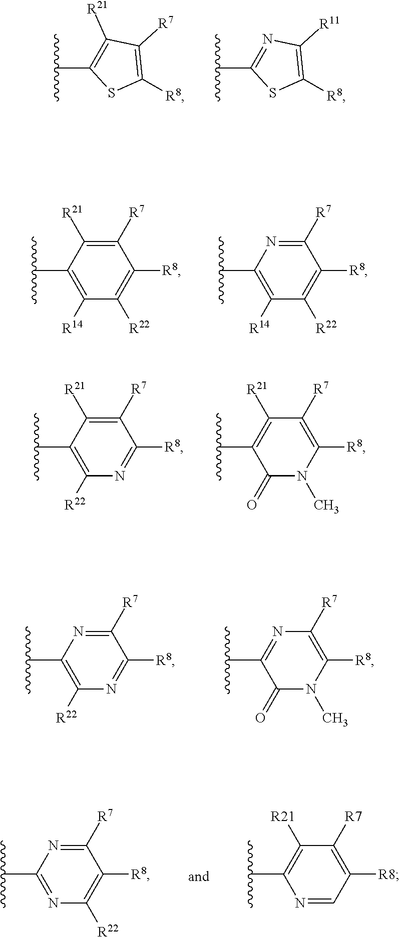
R¹ is selected from hydrogen, halogen, hydroxyl, C₁-C₆ alkyl, and C₁-C₆ alkoxy,
wherein the C₁-C₆ alkyl and C₁-C₆ alkoxy are optionally substituted with one or more halogens;
each R² and R³ is independently selected from hydrogen and C₁-C₆ alkyl,
wherein the C₁-C₆ alkyl is optionally substituted with one or more halogens;
or R² and R³ are taken together with the N to which they are attached to form a 3- to 10-membered heterocycle optionally substituted with one or more substituents independently selected from C₁-C₆ alkyl;
R⁴ is selected from hydrogen, halogen, C₁-C₆ alkyl, and C₁-C₆ alkoxy,
wherein the C₁-C₆ alkyl and C₁-C₆ alkoxy are optionally substituted with one or more substituents independently selected from halogens;
R⁸ is selected from —C(═O)OR¹⁵, —C(═O)NR²R³, —S(═O)₂NR²R³, —C(═O)NHR¹⁵, —CH₂OH, 3-hydroxyoxetan-3-yl, and —NH₂;
R⁶ is selected from hydrogen, halogen, hydroxyl, 5-membered heteroaryl, C₁-C₆ alkyl, —C(═O)OR¹⁵, —C(═O)R¹², —C(═O)NHR¹⁵, and —C(═O)N═S(═X³)(CH₃)₂,
wherein the C₁-C₆ alkyl are optionally substituted with one or more R⁹, and
wherein 5-membered heteroaryl contains at least two heteroatoms and is optionally substituted with one or more substituents independently selected from R¹⁷;
R⁷ is selected from hydrogen, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,
wherein the C₁-C₆ alkyl and C₁-C₆ alkoxy are optionally substituted with one or more halogens, and
wherein C₃-C₈ cycloalkyl, —O—C₃-C₈ cycloalkyl, 3- to 10-membered heterocycloalkyl, —O-(3- to 10-membered heterocycloalkyl), aryl and heteroaryl are optionally substituted with one or more R²⁴;
R⁸ is selected from hydrogen, —NO₂, C₁-C₆ alkyl, aryl, and heteroaryl,
wherein the C₁-C₆ alkyl and C₁-C₆ alkoxy are optionally substituted with one or more substituents independently selected at each occurrence from halogen; and
wherein aryl and heteroaryl are optionally substituted with one or more substituents independently selected at each occurrence from R²³;
or R⁷ and R⁸ are taken together to form a C₅-C₁₀ carbocycle or 5- to 10-membered heterocycle,
wherein C₅-C₁₀ carbocycle and 5- to 10-membered heterocycle are optionally substituted with one or more substituents independently selected from halogen, hydroxyl, —NO₂, C₁-C₆ alkyl, C₁-C₆ alkoxy, C₃-C₈ cycloalkyl, —O—C₃-C₈ cycloalkyl, 3- to 10-membered heterocycloalkyl, —O—(3- to 10-membered heterocycloalkyl), aryl, and heteroaryl,
wherein the C₁-C₆ alkyl and C₁-C₆ alkoxy are optionally substituted with one or more halogens, and
wherein aryl, heteroaryl, C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;
each R⁹ is independently selected from hydroxy and —COOH;
R¹⁰ is selected from —C(═O)—X¹—, —CH₂—X¹—, —X¹—C(═O)—, and —X¹—CH₂—;
R¹¹ is selected from hydrogen, —NO₂, C₁-C₆ alkyl, C₁-C₆ alkoxy, C₃-C₈ cycloalkyl, —O—C₃-C₈ cycloalkyl, 3- to 10-membered heterocycloalkyl, and —O—(3- to 10-membered heterocycloalkyl),
wherein the C₁-C₆ alkyl and C₁-C₆ alkoxy are optionally substituted with one or more halogens, and
wherein C₃-C₈ cycloalkyl, —O—C₃-C₈ cycloalkyl, 3- to 10-membered heterocycloalkyl, and —O—(3- to 10-membered heterocycloalkyl) are optionally substituted with one or more R²³;
R¹² is selected from alanine, arginine, asparagine, aspartic acid, asparagine, aspartic acid, cysteine, glutamic acid, glutamine, glutamine, glutamic acid, glycine, histidine, isoleucine, leucine, lysine, methionine, phenylalanine, proline, serine, threonine, tryptophan, tyrosine, and valine, wherein the point of attachment of R¹² is a nitrogen atom;
R¹⁴ is selected from hydrogen, halogen, hydroxyl, nitrile, —C(═O)CR¹⁵ and —C(═O)OR¹⁵;
each R¹⁵ is independently selected from hydrogen and C₁-C₆ alkyl, -heterocyclyl,
wherein the C₁-C₆ alkyl is optionally substituted with one or more substituents independently selected at each occurrence from —C(═O)NR²R³, -heterocyclyl, —NR²R³;
wherein the heterocyclyl is optionally substituted with one or more substituents independently selected at each occurrence from R² and R³;
R¹⁷ is selected from C₁-C₆ alkyl, aryl, and 6-membered heteroaryl,
wherein the C₁-C₆ alkyl is optionally substituted with one or more hydroxy, and
wherein aryl and 6-membered heteroaryl are optionally substituted with one or more substituents independently selected from halogen, —R², and —OR²;
R²⁰ is selected from hydrogen, halogen, hydroxyl, —COOH, —NC(═O)R², —OR², 5-membered heteroaryl, C₁-C₆ alkyl, —C(═O)N═S(═X³)(CH₃)₂, —CH₂(OH)CH₂OH and —NH—SO₂—R²,
wherein the 5-membered heteroaryl contains at least two heteroatoms, and
wherein the C₁-C₆ alkyl is optionally substituted with one or more substituents independently selected from 5-membered heteroaryl, wherein the 5-membered heteroaryl contains at least two heteroatoms;
R²¹ is selected from hydrogen and nitrile;
R²² is selected from hydrogen and hydroxy;
each R²³ is independently selected from halogen, C₁-C₆ alkyl, C₁-C₆ alkoxy,
wherein the C₁-C₆ alkyl and C₁-C₆ alkoxy are optionally substituted with one or more substituents independently selected from halogens;
each R²⁴ is independently selected from halogen, C₁-C₆ alkyl, C₁-C₆ alkoxy, 5-membered heteroaryl wherein the C₁-C₆ alkyl and C₁-C₆ alkoxy are optionally substituted with one or more substituents independently selected from halogens;
each X¹ is independently selected from —NR²— and —CR²R³—; and
each X³ is independently selected from NH and 0.

22. The method of claim 21, wherein R¹⁴ is selected from hydrogen and —C(═O)OR¹⁵, wherein R¹⁵
is selected from hydrogen and C₁-C₃ alkyl.

23. The method of claim 21, wherein, R¹⁴ is selected from hydrogen and —C(═O)OR¹⁵,
wherein R¹⁵ is selected from hydrogen and C₁-C₃ alkyl.

24. The method of claim 21, wherein R¹⁴ is —COOH.

25. The method of claim 17, wherein the small molecule is selected from the group consisting of:

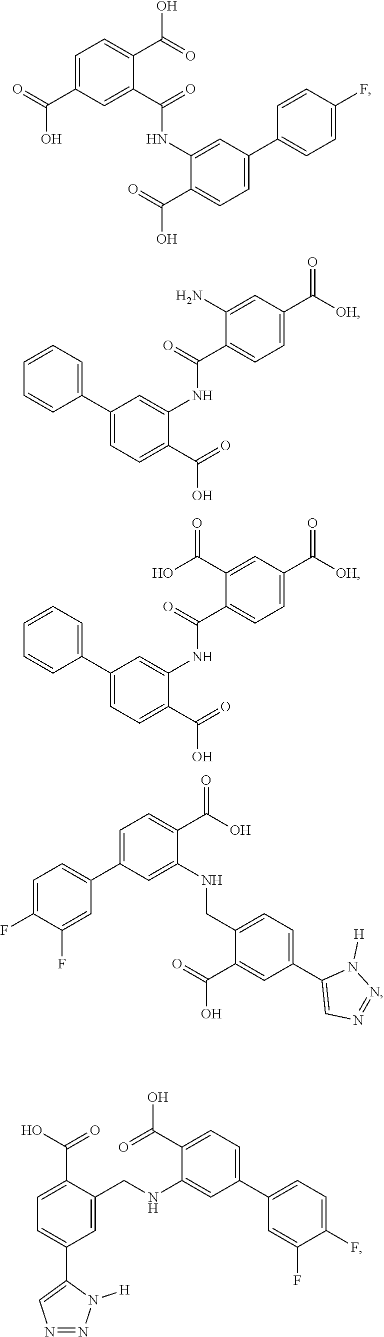

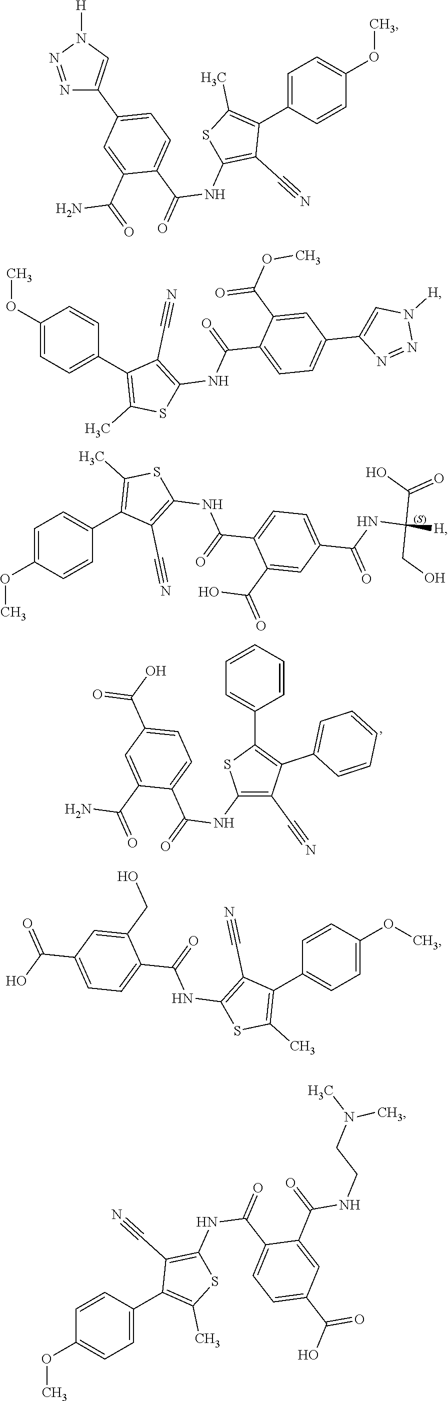

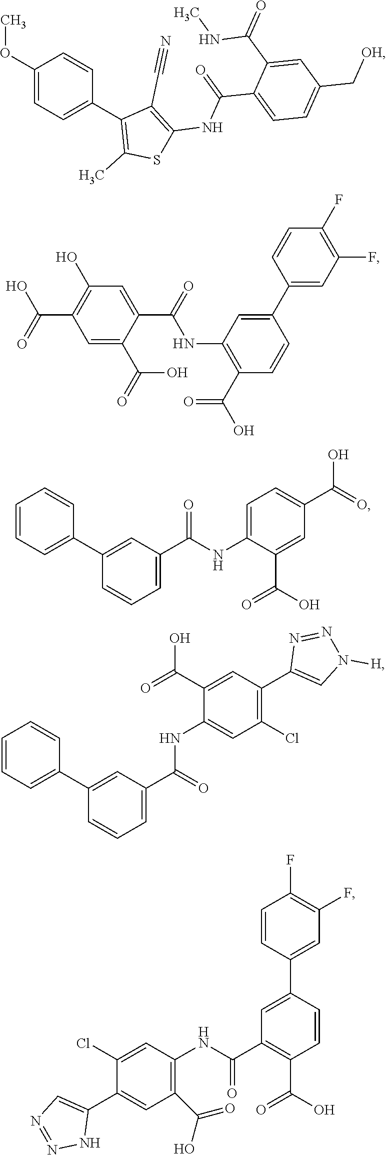

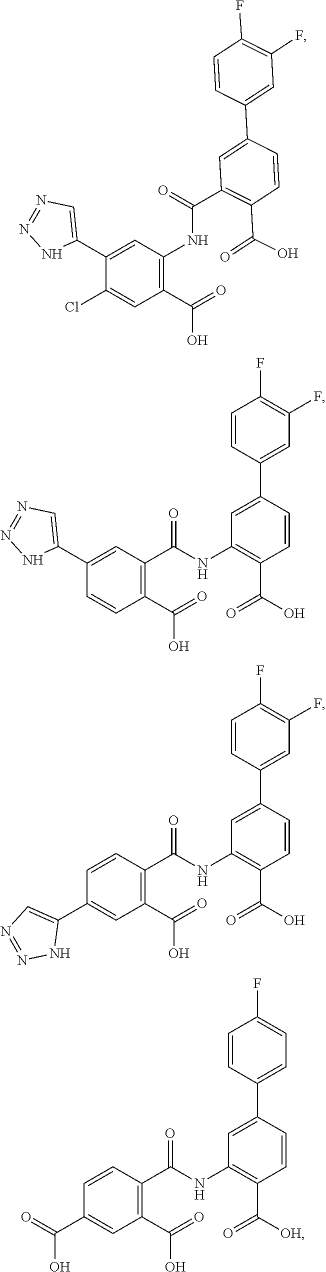 [SMILES: O=C(O)c1cc(Cl)c(-c2cnn[nH]2)cc1NC(=O)c1cc(-c2ccc(F)c(F)c2)ccc1C(=O)O ; O=C(O)c1ccc(-c2ccc(F)c(F)c2)cc1NC(=O)c1cc(-c2cnn[nH]2)ccc1C(=O)O ; O=C(O)c1ccc(-c2ccc(F)c(F)c2)cc1NC(=O)c1ccc(-c2cnn[nH]2)cc1C(=O)O ; O=C(O)c1ccc(C(=O)Nc2cc(-c3ccc(F)cc3)ccc2C(=O)O)c(C(=O)O)c1]

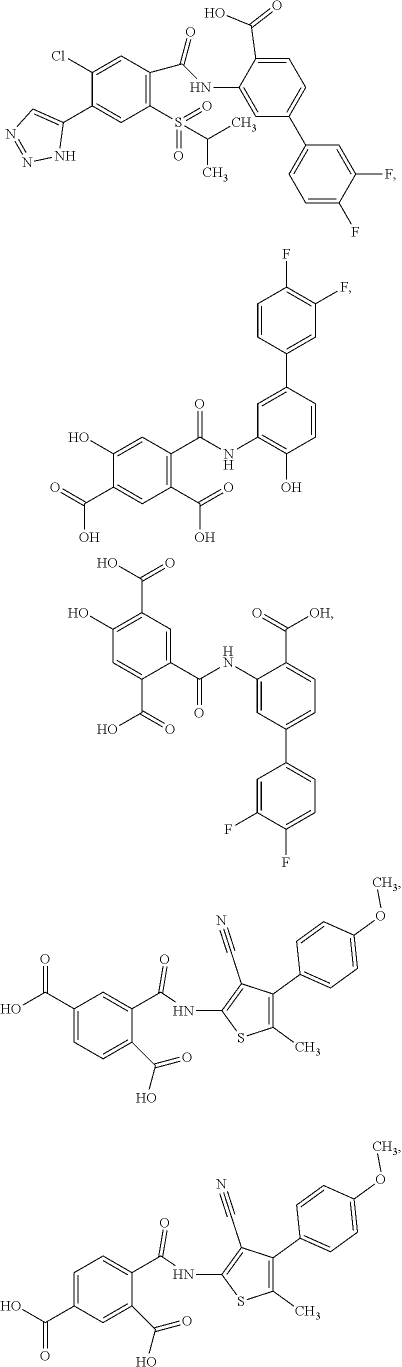

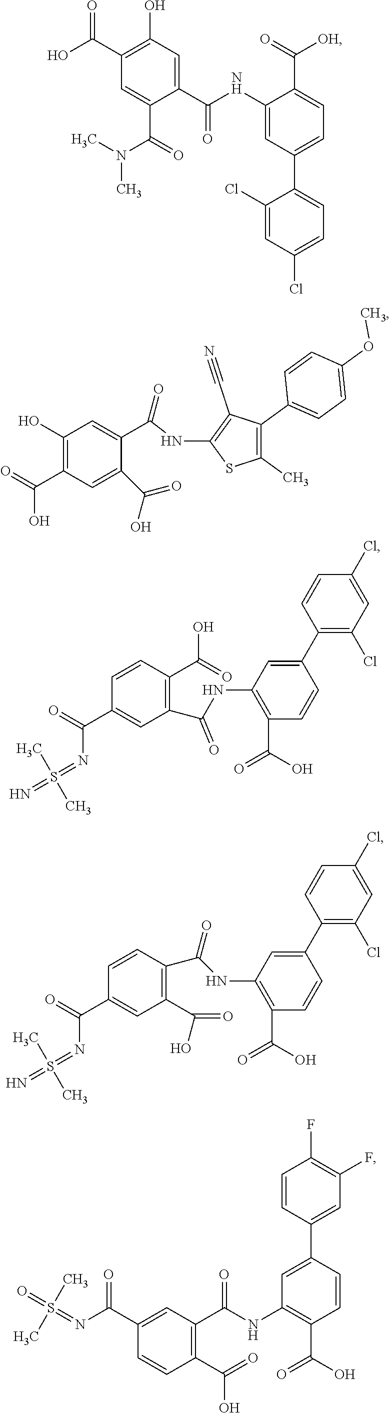 [SMILES: CN(C)C(=O)c1cc(C(=O)O)c(O)cc1C(=O)Nc1cc(-c2ccc(Cl)cc2Cl)ccc1C(=O)O ; COc1ccc(-c2c(C)sc(NC(=O)c3cc(O)c(C(=O)O)cc3C(=O)O)c2C#N)cc1 ; CS(C)(=N)=NC(=O)c1ccc(C(=O)O)c(C(=O)Nc2cc(-c3ccc(Cl)cc3Cl)ccc2C(=O)O)c1 ; CS(C)(=N)=NC(=O)c1ccc(C(=O)Nc2cc(-c3ccc(Cl)cc3Cl)ccc2C(=O)O)c(C(=O)O)c1 ; CS(C)(=O)=NC(=O)c1ccc(C(=O)O)c(C(=O)Nc2cc(-c3ccc(F)c(F)c3)ccc2C(=O)O)c1]

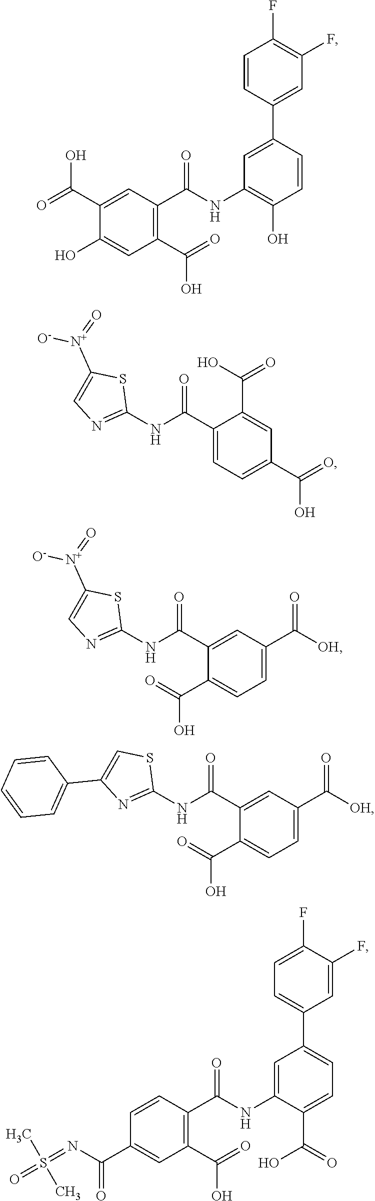

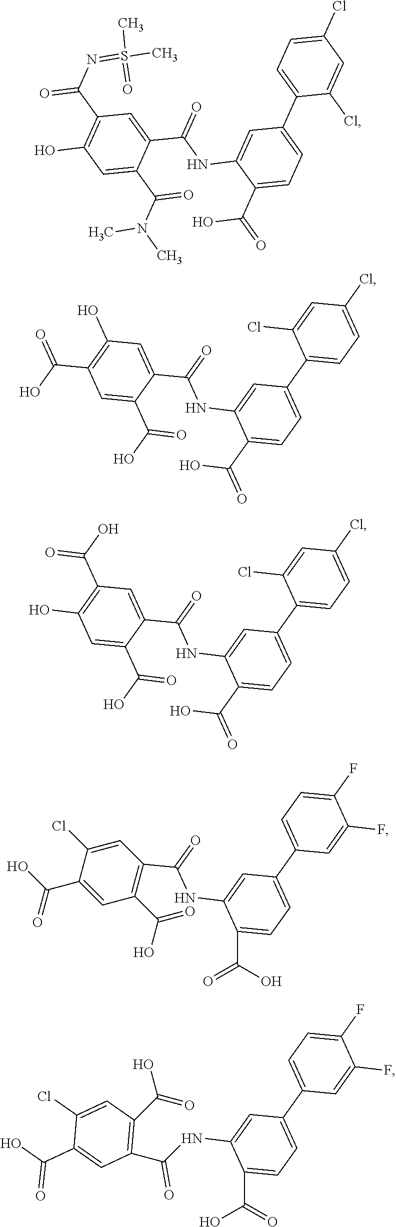 [SMILES: CN(C)C(=O)c1cc(O)c(C(=O)N=S(C)(C)=O)cc1C(=O)Nc1cc(-c2ccc(Cl)cc2Cl)ccc1C(=O)O ; O=C(O)c1cc(C(=O)O)c(C(=O)Nc2cc(-c3ccc(Cl)cc3Cl)ccc2C(=O)O)cc1O ; O=C(O)c1cc(C(=O)Nc2cc(-c3ccc(Cl)cc3Cl)ccc2C(=O)O)c(C(=O)O)cc1O ; O=C(O)c1cc(C(=O)O)c(C(=O)Nc2cc(-c3ccc(F)c(F)c3)ccc2C(=O)O)cc1Cl ; O=C(O)c1cc(C(=O)Nc2cc(-c3ccc(F)c(F)c3)ccc2C(=O)O)c(C(=O)O)cc1Cl]

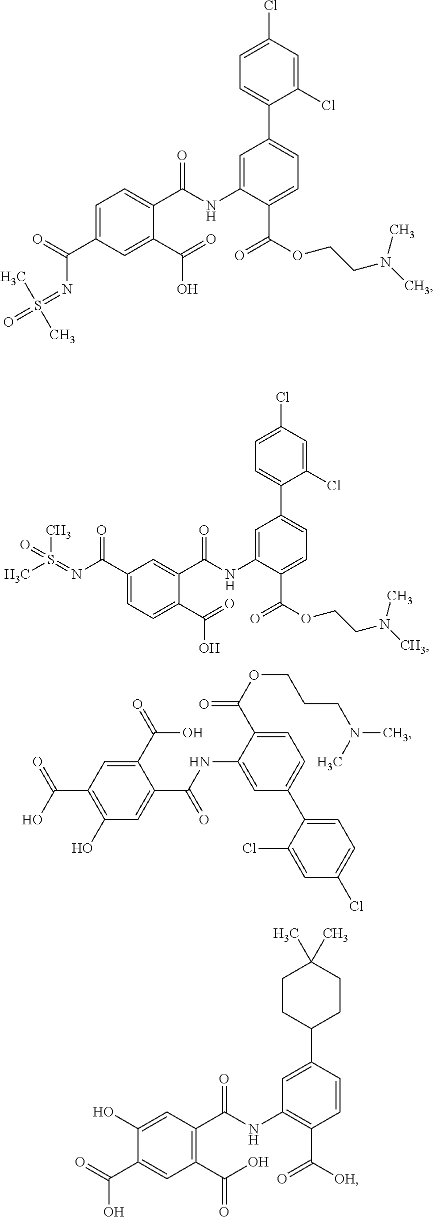

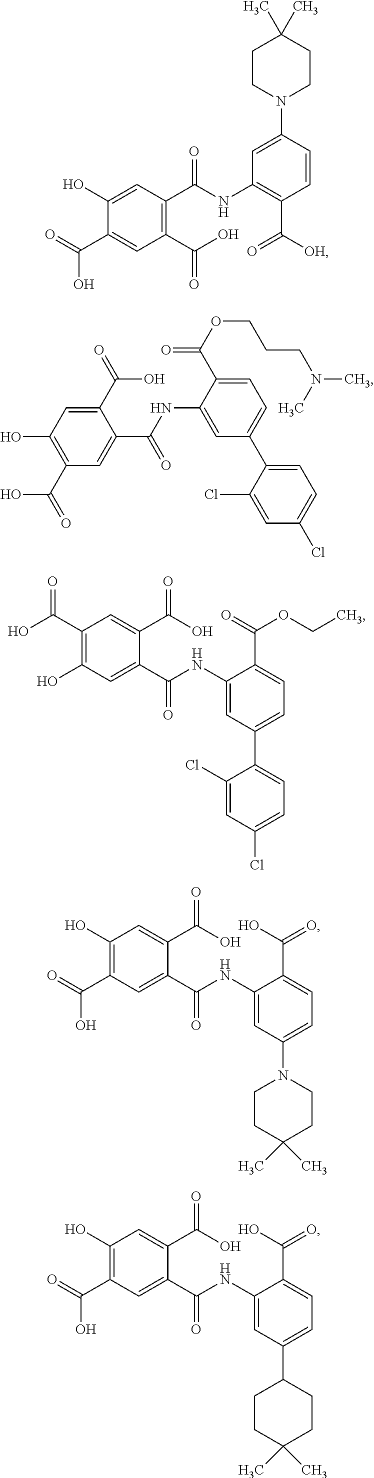

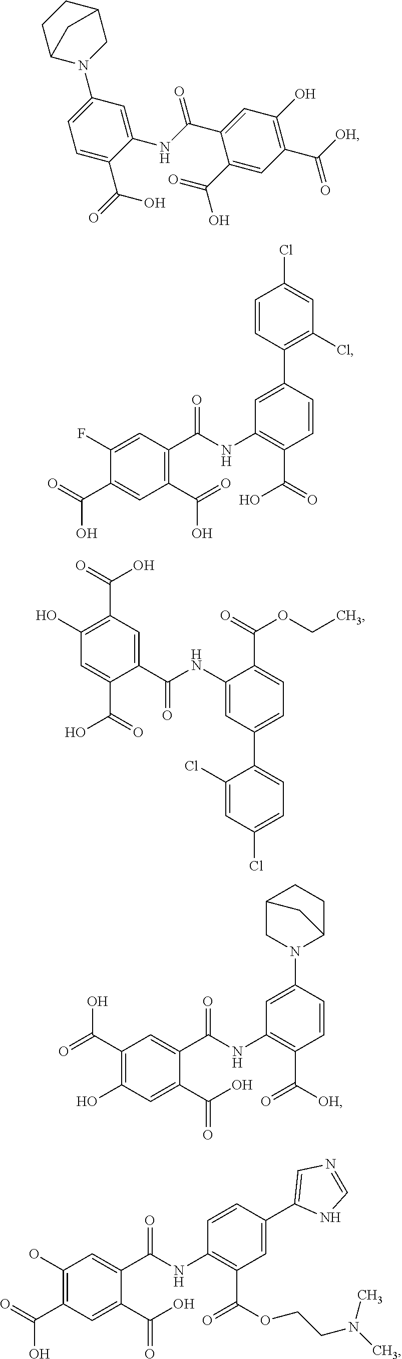

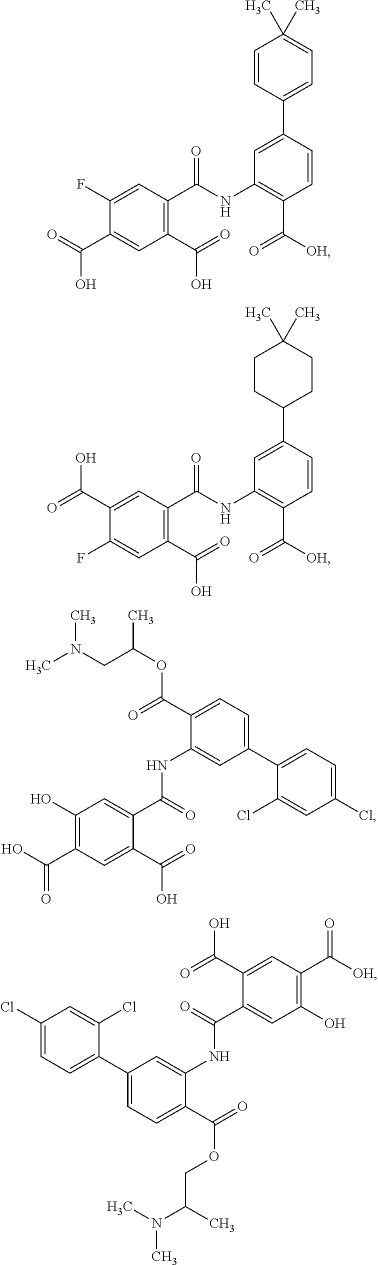

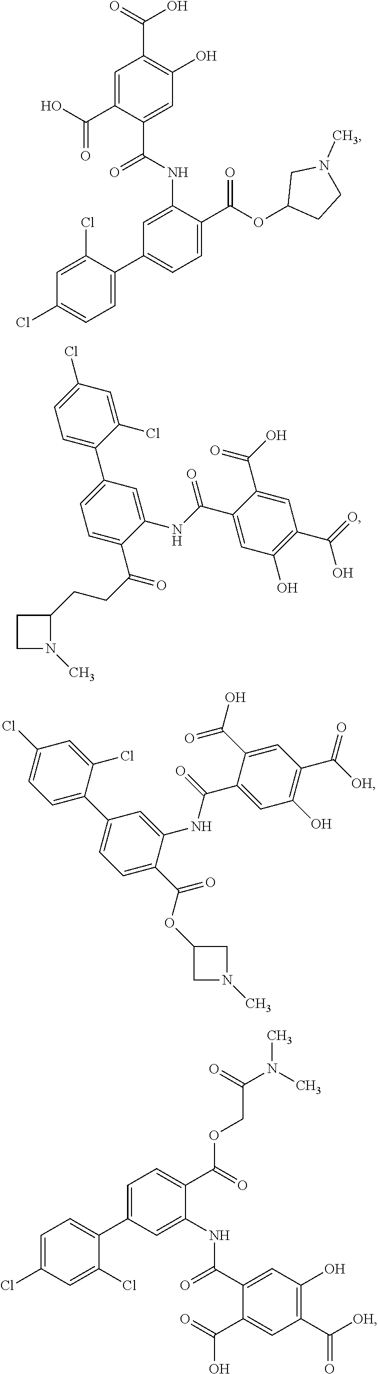

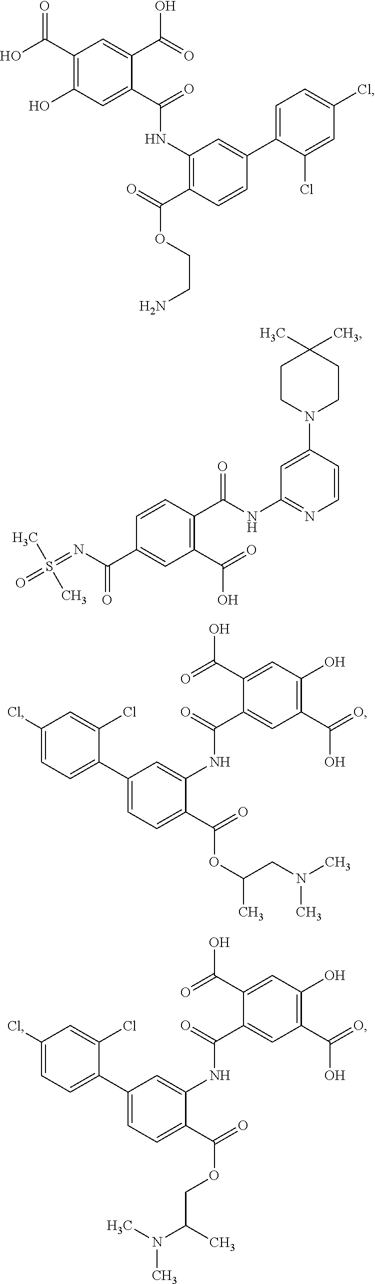

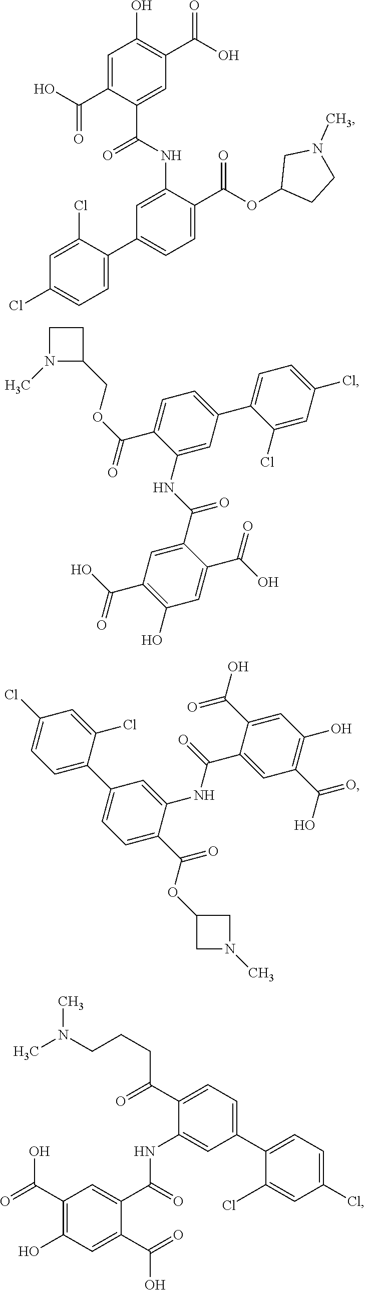

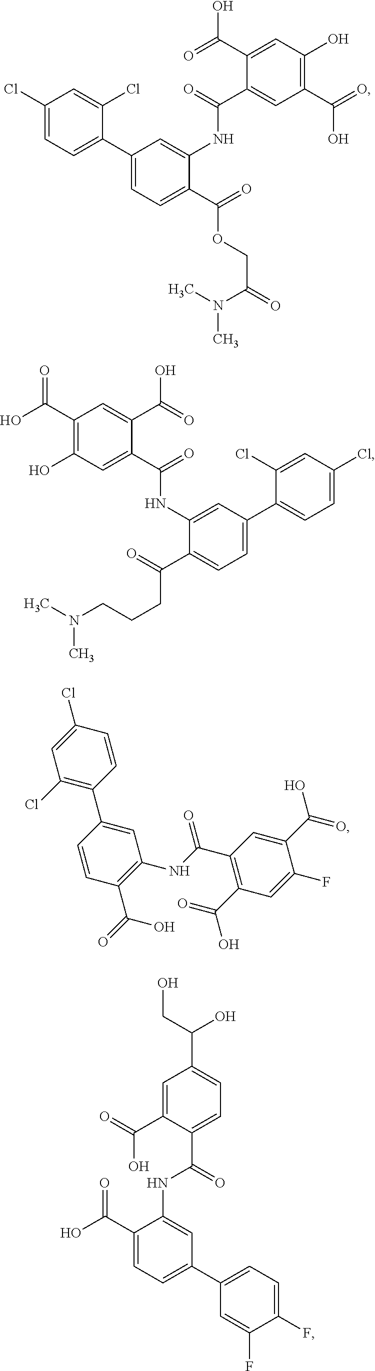

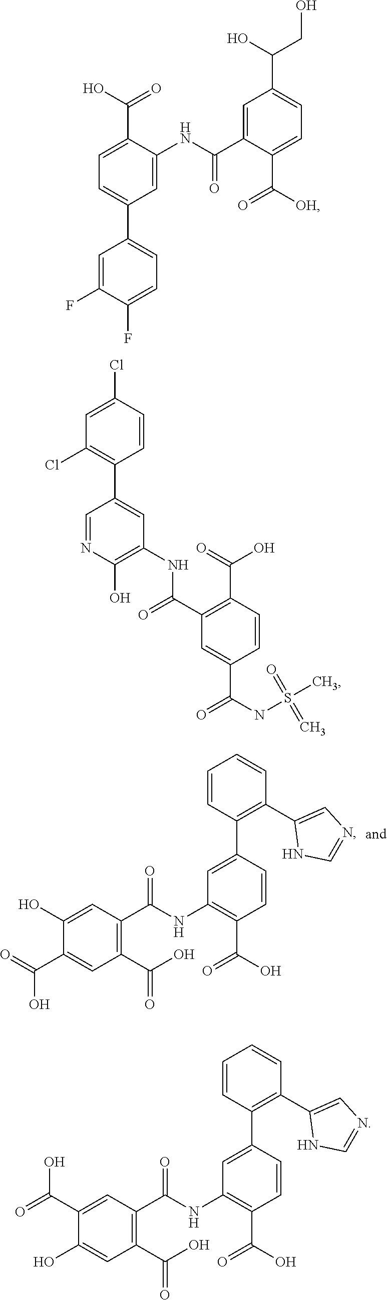

26. The method of claim 17, wherein the small molecule is a compound of formula (VIII): 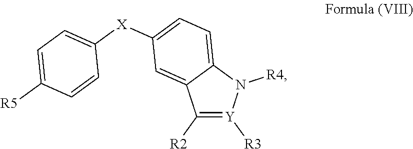
or a pharmaceutically acceptable salt thereof, wherein: each X is independently selected from —O—, —S—, —NR⁷— or —CR⁷R⁸—; each Y is independently selected from C or N; each R⁷ and R⁸ is independently selected from hydrogen and C₁-C₆ alkyl, C₁-C₆ alkoxy, wherein said alkyl is optionally substituted with one or more halogens;
R² is selected from hydrogen, halogens, nitrile and 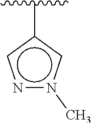
R³ is selected from hydrogen and —NR⁷R⁸
R⁴ is selected from hydrogen, C₁-C₆ alkyl, C₁-C₆ alkoxy, aryl, heteroaryl, C₃-C₈cycloalkyl, 10-membered heterocycloalkyl, 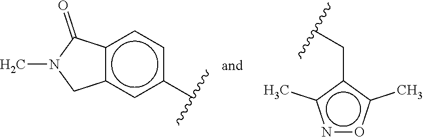
wherein the C₁-C₆alkyl and C₁-C₆alkoxy are optionally substituted with one or more substituents independently selected at each occurrence from halogens, —C(═O)NR⁷R⁸ and R²; and
wherein heteroaryl, C₃-C₈ cycloalkyl, —O—C₃-C₈ cycloalkyl, 3- to 10-memberedheterocycloalkyl and —O—(3- to 10-memberedheterocycloalkyl) are optionally substituted with one or more R²;
R⁸ is selected from 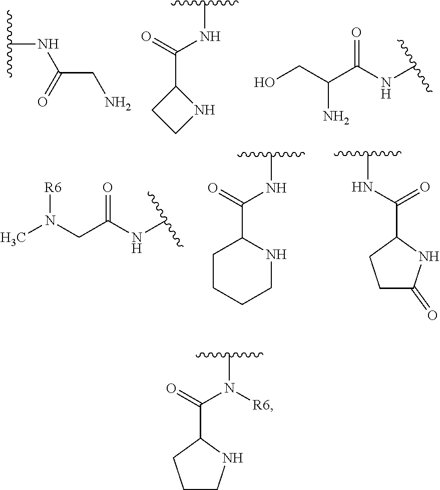
R⁶ is selected from hydrogen and C₁-C₆ alkyl, or a pharmaceutically acceptable salt thereof, or N-oxide, or solvate, or tautomer, or stereoisomer, or racemate, including mixtures thereof in all ratios.

27. The method of claim 17, wherein the small molecule is selected from the group consisting of:
(2RS)—N-[4-(13-cyano-1-[(3,5dimethyl-1,2-oxazol-4-yl)methyl]-1H-indol-5-yl}oxy)phenyl]pyrrolidine-2-carboxamide, N-[4-(13-cyano-1-[(3,5-dimethyl-1,2-oxazol-4-yl)methyl]-1H-indol-5-yl}oxy)phenyl]pyrrolidine-2-carboxamide, (2S)—N-[4-(13-cyano-1-[(3,5-dimethyl-1,2-oxazol-4-yl)methyl]-1H-indol-5-yl}oxy)phenyl]pyrrolidine-2-carboxamide, (2S)—N-{4-[(3-cyano-1-ethyl-1H-indol-5-yl)oxy]phenyl}-5-oxopyrrolidine-2-carboxamide, 2-amino-N-(4-{[3-(1-methyl-1H-pyrazol-4-yl)-1H-indol-5-yl]oxy}phenyl)acetamide, (2S)—N-(4-{[1-methyl-3-(i-methyl-1H-pyrazol-4-yl)-1H-indol-5-yl]oxy}phenyl)pyrrolidine-2-carboxamide, (2S)—N-(4-{[3-cyano-1-(2-methylpropyl)-1H-indol-5-yl]amino}phenyl)pyrrolidine-2-carboxamide, (2S)—N-(4-{[3-cyano-1-(2-methylpropyl)-1H-indol-5-yl](methyl)amino}phenyl)pyrrolidine-2-carboxamide, (2S)—N-(4-{[3-cyano-1-(2-methylpropyl)-1H-indol-5-yl]sulfanyl}phenyl)pyrrolidine-2-carboxamide, (2S)—N-(4-{[3-cyano-1-(2-methylpropyl)-1H-indol-5-yl]sulfonyl}phenyl)pyrrolidine-2-carboxamide, (2S)—N-(4-{[3-cyano-1-(2-methylpropyl)-1H-indol-5-yl]methyl}phenyl)pyrrolidine-2-carboxamide (2S)—N-(4-{[3-cyano-1-(2-methylpropyl)-1H-indol-5-yl]oxy}phenyl)pyrrolidine-2-carboxamide, 2-amino-N-14-[(2-amino-3-cyano-1-ethyl-1H-indol-5-yl)oxy]phenyl}acetamide, 2-amino-N-14-[(2-amino-3-cyano-1-methyl-1H-indol-5-yl)oxy]phenyl}acetamide, (2S)-2-amino-N-14-[(2-amino-3-cyano-1H-indol-5-yl)oxy]phenyl}-3-hydroxypropanamide, 2-amino-N-14-[(2-amino-3-cyano-1H-indol-5-yl)oxy]phenyl}acetamide, 2-amino-N-(4-{[2-amino-3-cyano-1-(2-methylpropyl)-1H-indol-5-yl]oxy}phenyl)acetamide, 2-amino-N-14-[(2-amino-1-benzyl-3-cyano-1H-indol-5-yl)oxy]phenyl}acetamide, 2-12-amino-5-[4-(2-aminoacetamido)phenoxy]-3-cyano-1H-indol-1-yl}-N,N-dimethylacetamide, 2-amino-N-14-[(3-cyano-1H-indol-5-yl)oxy]phenyl}acetamide, 2-amino-N-14-[(3-cyano-1-ethyl-1H-indol-5-yl)oxy]phenyl}acetamide, N-14-[(3-cyano-1-ethyl-1H-indol-5-yl)oxy]phenyl}-2-(methylamino)acetamide, N-14-[(3-cyano-1-ethyl-1H-indol-5-yl)oxy]phenyl}-2-(dimethylamino)acetamide, (2S)—N-{4-[(3-cyano-1-ethyl-1H-indol-5-yl)oxy]phenyl}pyrrolidine-2-carboxamide, (2R)-N-14-[(3-cyano-1-ethyl-1H-indol-5-yl)oxy]phenyl}pyrrolidine-2-carboxamide, (2S)—N-{4-[(3-cyano-1-ethyl-1H-indol-5-yl)oxy]phenyl}-N-methylpyrrolidine-2-carboxamide, (2S)—N-{4-[(3-cyano-1-ethyl-1H-indol-5-yl)oxy]phenyl}azetidine-2-carboxamide, (2S)—N-{4-[(3-cyano-1-ethyl-1H-indol-5-yl)oxy]phenyl}pyrrolidine-2-carboxamide, (2R)-N-14-[(3-cyano-1-ethyl-1H-indol-5-yl)oxy]phenyl}pyrrolidine-2-carboxamide, (2S)—N-{4-[(3-cyano-1-ethyl-1H-indol-5-yl)oxy]phenyl}-N-methylpyrrolidine-2-carboxamide, (2S)—N-{4-[(3-cyano-1-ethyl-1H-indol-5-yl)oxy]phenyl}azetidine-2-carboxamide, (2S)—N-{4-[(3-cyano-1-ethyl-1H-indol-5-yl)oxy]phenyl}piperidine-2-carboxamide, (2S)—N-{4-[(3-cyano-1-ethyl-1H-indol-5-yl)oxy]phenyl}-N-methyl-5-oxopyrrolidine-2-carboxamide, (2S)—N-[4-(13-cyano-1-[(methylcarbamoyl)methyl]-1H-indol-5-yl}oxy)phenyl]pyrrolidine-2-carboxamide (2S)—N-[4-(13-cyano-1-[2-(dimethylamino)ethyl]-1H-indol-5-yl}oxy)phenyl]pyrrolidine-2-carboxamide, (2S)—N-[4-(13-cyano-1-[(oxan-4-yl)methyl]-1H-indol-5-yl}oxy)phenyl]pyrrolidine-2-carboxamide, (2S)—N-{4-[(3-cyano-1-phenyl-1H-indol-5-yl)oxy]phenyl}pyrrolidine-2-carboxamide, (2S)—N-(4-{[3-cyano-1-(2-methyl-1-oxo-2,3-dihydro-1H-isoindol-5-yl)-1H-indol-5-yl]oxy}phenyl)pyrrolidine-2-carboxamide, (2S)—N-14-[(1-benzyl-3-cyano-1H-indol-5-yl)oxy]phenyl}pyrrolidine-2-carboxamide, (2S)—N-[4-(13-cyano-1-[(3,5-dimethyl-1,2-oxazol-4-yl)methyl]-1H-indol-5-yl}oxy)phenyl]pyrrolidine-2-carboxamide, (2S)—N-(4-{[3-cyano-1-(2-methylpropyl)-1H-indazol-5-yl]oxy}phenyl)pyrrolidine-2-carboxamide, (2S)—N-[4-({3-cyano-1-[(3,5-dimethyl-1,2-oxazol-4-yl)methyl]-1H-indazol-5-yl}oxy)phenyl]pyrrolidine-2-carboxamide,2-amino-N-(4-{[3-(1-methyl-1H-pyrazol-4-yl)-1H-indazol-5-yl]oxy}phenyl)acetamide, (2S)—N-(4-{[3-(1-methyl-1H-pyrazol-4-yl)-1H-indol-5-yl]oxy}phenyl)pyrrolidine-2-carboxamide, N-(4-{[3-cyano-1-(2-methylpropyl)-1H-indol-5-yl]oxy}phenyl)pyrrolidine-2-carboxamide or a pharmaceutically acceptable salt thereof, or solvate, or tautomer, or stereoisomer, or racemate, including mixtures thereof in all ratios.

28. The method of claim 17, wherein the small molecule is a compound of formula (IX) 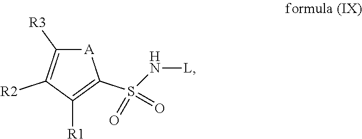
wherein: (i) A is O or S; and
R¹ is selected from H; halogen; and C1-C6 alkyl optionally substituted with at least one halogen; or
R¹ and R² form, together with the carbon atoms to which they are attached, a benzene ring optionally substituted with at least one R⁶; or a 5- or 6-membered heteroaromatic or heterocyclic ring, optionally substituted with at least one R⁶;
R² and R³ are independently selected from H; halogen; C1-C6 alkyl optionally substituted with at least one halogen; phenyl optionally substituted with at least one R⁶; 5- or 6-membered heteroaryl optionally substituted with at least one R⁶; and 5- or 6-membered arylsulfonyl or heteroaryl sulfonyl, optionally substituted with at least one R⁶; provided that
at least one of R² and R³ is selected from said phenyl, heteroaryl, arylsulfonyl and heteroarylsulfonyl, and
when L is (a), neither R² nor R³ is unsubstituted phenyl; or
R² and R³ form, together with the carbon atoms to which they are attached, a benzene ring optionally substituted with at least one R⁶; or a 5- or 6-membered heteroaromatic or hetero-cyclic ring, optionally substituted with at least one R⁶; or
(ii) A is CR′═CR′;
each R′ is independently selected from H; halogen; and C1-C6 alkyl optionally substituted with at least one halogen;
R¹ is selected from H; halogen; and C1-C6 alkyl optionally substituted with at least one halogen; or
R¹ and R² form, together with the carbon atoms to which they are attached, a benzene ring optionally substituted with at least one R⁶; or a 5- or 6-membered heteroaromatic or heterocyclic ring, optionally substituted with at least one R⁶;
R² and R³ are independently selected from H, halogen, C1-C6 alkyl optionally substituted with at least one halogen; phenyl optionally substituted with at least one R⁶; 5- or 6-membered heteroaryl optionally substituted with at least one R⁶; and 5- or 6-membered aryl-sulfonyl or heteroaryl sulfonyl, optionally substituted with at least one R⁶; provided that:
when both R² and R³ are selected from H, halogen and C1-C6 alkyl optionally substituted with at least one halogen, the ring containing A is substituted in ortho position relative to the sulphonamide bond with at least one substituent selected from halogen and C1-C6 alkyl optionally substituted with at least one halogen;
when L is (a), neither R² nor R³ is unsubstituted phenyl; and
when L is (c), R³ is optionally substituted phenyl only when R⁵ is tetrazol-5-yl, and R² is unsubstituted phenyl only when R⁴ is not hydroxy; or
R² and R³ form, together with the carbon atoms to which they are attached, a benzene ring optionally substituted with at least one R⁶, provided that said benzene ring is unsubstituted only when R⁸ is tetrazolyl or oxazolyl; or a 5- or 6-membered heteroaromatic or heterocyclic ring, optionally substituted with at least one R⁶;
L is 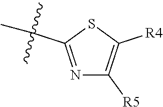
wherein R⁴ is COOR¹²; and R⁵ is selected from H and C1-C6 alkyl; or
R⁴ is selected from H and C1-C6 alkyl; and R⁸ is COOR¹²; or
(b) 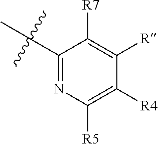
wherein R⁴ is selected from H and C1-C6 alkyl; R⁸ is selected from H and C1-C6 alkyl; and R″ is selected from C0-C1 alkyl-COOR¹²; or
R⁸ is selected from COOR¹²; and R″ is selected from H and C1-C6 alkyl; and
R is selected from H, C1-C6 alkyl, and nitro;
(c) 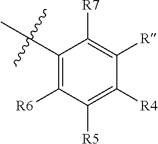
wherein R⁴ is selected from H, hydroxy and C1-C6 alkyl; R⁵ is selected from H, C1-C6 alkyl; and R″ is selected from C0-C1 alkyl-COOR¹²; or
R⁸ is selected from COOR¹², oxazol-5-yl and tetrazol-5-yl, said oxazol-5-yl and tetrazol-5-yl optionally being substituted by R⁹; and R″ is selected from H, C1-C6 alkyl, and nitro;
R⁷ is selected from H, C1-C6 alkyl, and nitro; and
R is selected from H, hydroxy, and C1-C6 alkyl; or
(d) 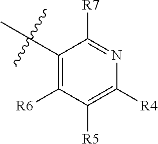
wherein R⁴ is selected from H and C1-C6 alkyl; and R⁵ is COOR¹²; R⁷ is selected from H, C1-C6 alkyl, and nitro; and R⁸ is selected from H, hydroxy, and C1-C6 alkyl;
provided that in any of (a), (b), (c) and (d), R⁴, R⁵ and R″ are selected from C0-C1 alkyl-COOR¹² only when at least one of R² or R³ is optionally substituted phenyl or optionally substituted heteroaryl; or when R² and R³ together with the carbon atoms to which they are attached form a benzene ring optionally substituted by at least one R⁶;
R⁶ is selected from C1-C6 alkyl, cyano, halogen, hydroxy, C1-C6 alkoxy, C1-C6 alkylthio, tetrahydropyrrolyl, R¹⁰R¹¹N, carbamoyl, and C1-C6 alkylcarbonylamino, or is an ethyleneoxy biradical forming, together with the atoms to which it is attached, a five-membered oxygen containing cycle; wherein any alkyl is optionally substituted with at least one halogen;
R⁹ is selected from C0-C1 alkyl-COOR¹²;
R¹⁰ and R¹¹ are independently selected from H and C1-C6 alkyl or form, together with the nitrogen to which they are attached, a 5- or 6-membered cyclic amino optionally containing one other cyclic heteroatom;
R¹² is selected from H, C1-C6 alkyl; heteroaryl-C0-C2 alkyl; (C1-C3 alkoxy)ₚC1-C3 alkyl; aryl-C0-C2 alkyl; heterocyclyl-C0-C2 alkyl; and C1-C6 dialkylamino-C1-C6 alkyl, wherein any cyclic moiety is optionally substituted with C1-C6 alkyl;
p is 1 or 2;
or a pharmaceutically acceptable salt thereof;
provided that the compound is not:
ethyl 2-(benzofuran-2-sulfonamido)thiazole-4-carboxylate;
ethyl 2-(5-methylbenzo[b]thiophene-2-sulfonamido)thiazole-4-carboxylate;
ethyl 2-(benzo[b]thiophene-2-sulfonamido)thiazole-4-carboxylate;
ethyl 2-(6-acetamidonaphthalene-2-sulfonamido)-4-methylthiazole-5-carboxylate;
ethyl 2-(6-aminonaphthalene-2-sulfonamido)-4-methylthiazole-5-carboxylate;
methyl 6-(4′-cyano-[1,1′-biphenyl]-4-ylsulfonamido)picolinate;
2-(3-(benzo[b]thiophene-2-sulfonamido)phenyl)acetic acid;
methyl 2-(3-(benzo[b]thiophene-2-sulfonamido)phenyl)acetate;
ethyl 3-(5-(6-oxo-1,6-dihydropyridazin-3-yl)furan-2-sulfonamido)benzoate;
ethyl 3-(5-(5-(trifluoromethyl)isoxazol-3-yl)furan-2-sulfonamido)benzoate;
ethyl 3-(5-(4,5-dimethyl-1H-pyrazol-3-yl)thiophene-2-sulfonamido)benzoate;
ethyl 3-(5-(5-methyl-1H-pyrazol-3-yl)thiophene-2-sulfonamido)benzoate;
ethyl 3-(5-(5-(trifluoromethyl)isoxazol-3-yl)thiophene-2-sulfonamido)benzoate;
ethyl 3-(5-(3-(trifluoromethyl)isoxazol-5-yl)thiophene-2-sulfonamido)benzoate;
ethyl 3-(5-(3-methylisoxazol-5-yl)thiophene-2-sulfonamido)benzoate;
ethyl 3-(4-(4-(tert-butyl)thiazol-2-yl)thiophene-2-sulfonamido)benzoate;
methyl 3-(4-(4-(tert-butyl)thiazol-2-yl)thiophene-2-sulfonamido)benzoate;
methyl 3-(3-(1H-tetrazol-1-yl)phenylsulfonamido)benzoate;
ethyl 3-(2-ethyl-5-(5-(trifluoromethyl)isoxazol-3-yl)phenylsulfonamido)benzoate;
ethyl 3-(2-methyl-5-(5-methyl-1,2,4-oxadiazol-3-yl)phenylsulfonamido)benzoate;
ethyl 3-(3-(5-methyl-1,2,4-oxadiazol-3-yl)phenylsulfonamido)benzoate;
ethyl 3-(2-methyl-5-(5-methyl-1H-pyrazol-3-yl)phenylsulfonamido)benzoate;
ethyl 3-(2-methyl-5-(2-methylthiazol-4-yl)phenylsulfonamido)benzoate;
ethyl 3-(2-isopropyl-5-(3-methylisoxazol-5-yl)phenylsulfonamido)benzoate;
ethyl 3-(2-methyl-5-(2-methyloxazol-5-yl)phenylsulfonamido)benzoate;
ethyl 3-(2-ethyl-5-(3-methylisoxazol-5-yl)phenylsulfonamido)benzoate;
ethyl 3-(4-(2-methyloxazol-4-yl)phenylsulfonamido)benzoate;
ethyl 3-(4-(2-methyloxazol-5-yl)phenylsulfonamido)benzoate;
methyl 3-(4-(2,5-dimethyloxazol-4-yl)phenylsulfonamido)benzoate;
ethyl 3-(2-methyl-5-(6-oxo-1,6-dihydropyridazin-3-yl)phenylsulfonamido)benzoate;
3-(6-butoxynaphthalene-2-sulfonamido)benzoic acid;
3-(6-methoxynaphthalene-2-sulfonamido)benzoic acid;
3-(6-propoxynaphthalene-2-sulfonamido)benzoic acid;
3-(6-methylnaphthalene-2-sulfonamido)benzoic acid;
3-(4-(3,5-dimethyl-1H-pyrazol-1-yl)phenylsulfonamido)benzoic acid;
N-(3-(2H-tetrazol-5-yl)phenyl)benzo[c][1,2,5]thiadiazole-4-sulfonamide;
N-(3-(2H-tetrazol-5-yl)phenyl)-2,3,5,6-tetramethylbenzenesulfonamide;
N-(3-(2H-tetrazol-5-yl)phenyl)-2,4,5-trichlorobenzenesulfonamide;
N-(3-(2H-tetrazol-5-yl)phenyl)-5-(tert-butyl)-2-methylbenzenesulfonamide;
3-methyl-N-(3-(oxazol-5-yl)phenyl)quinoline-8-sulfonamide;
5-bromo-2-methyl-N-(3-(oxazol-5-yl)phenyl)benzenesulfonamide;
2,5-dichloro-3,6-dimethyl-N-(3-(oxazol-5-yl)phenyl)benzenesulfonamide;
N-(3-(oxazol-5-yl)phenyl)-2,3-dihydrobenzofuran-5-sulfonamide;
2-chloro-4-methyl-N-(3-(oxazol-5-yl)phenyl)benzenesulfonamide;
2-chloro-4-fluoro-N-(3-(oxazol-5-yl)phenyl)benzenesulfonamide;
2-fluoro-N-(3-(oxazol-5-yl)phenyl)benzenesulfonamide;
N-(3-(oxazol-5-yl)phenyl)quinoline-8-sulfonamide;
N-(3-(oxazol-5-yl)phenyl)naphthalene-2-sulfonamide;
2-bromo-N-(3-(oxazol-5-yl)phenyl)benzenesulfonamide;
5-(dimethylamino)-N-(3-(oxazol-5-yl)phenyl)naphthalene-1-sulfonamide;
2,3,5, 6-tetramethyl-N-(3-(oxazol-5-yl)phenyl)benzenesulfonamide;
2,5-dichloro-N-(3-(oxazol-5-yl)phenyl)benzenesulfonamide; or
2,3,4-trifluoro-N-(3-(oxazol-5-yl)phenyl)benzenesulfonamide.

29. The method of claim 17, wherein the small molecule is a compound of formula (IX) wherein:
(i) A is O or S; and
R¹ is selected from H; halogen; and C1-C6 alkyl optionally substituted with at least one halogen; or
R¹ and R² form, together with the carbon atoms to which they are attached, a benzene ring optionally substituted with at least one R⁶; or a 5- or 6-membered heteroaromatic or heterocyclic ring, optionally substituted with at least one R⁶;
R² and R³ are independently selected from H; halogen; C1-C6 alkyl optionally substituted with at least one halogen; phenyl optionally substituted with at least one R⁶; 5- or 6-membered heteroaryl optionally substituted with at least one R⁶; and 5- or 6-membered arylsulfonyl or heteroaryl sulfonyl, optionally substituted with at least one R⁶; provided that
at least one of R² and R³ is selected from said phenyl, heteroaryl, arylsulfonyl and heteroarylsulfonyl, and -when L is (a), neither R² nor R³ is unsubstituted phenyl; or
R² and R³ form, together with the carbon atoms to which they are attached, a benzene ring optionally substituted with at least one R⁶; or a 5- or 6-membered heteroaromatic or hetero-cyclic ring, optionally substituted with at least one R⁶; or
(ii) A is CR′═CR′;
each R′ is independently selected from H; halogen; and C1-C6 alkyl optionally substituted with at least one halogen;
R¹ is selected from H; halogen; and C1-C6 alkyl optionally substituted with at least one halogen; or
R¹ and R² form, together with the carbon atoms to which they are attached, a benzene ring optionally substituted with at least one R⁶; or a 5- or 6-membered heteroaromatic or heterocyclic ring, optionally substituted with at least one R⁶;
R² and R³ are independently selected from H, halogen, C1-C6 alkyl optionally substituted with at least one halogen; phenyl optionally substituted with at least one R⁶; 5- or 6-membered heteroaryl optionally substituted with at least one R⁶; and 5- or 6-membered aryl-sulfonyl or heteroaryl sulfonyl, optionally substituted with at least one R⁶; provided that:
when both R² and R³ are selected from H, halogen and C1-C6 alkyl optionally substituted with at least one halogen, the ring containing A is substituted in ortho position relative to the sulphonamide bond with at least one substituent selected from halogen and C1-C6 alkyl optionally substituted with at least one halogen;
when L is (a), neither R² nor R³ is unsubstituted phenyl; and
when L is (c), R³ is optionally substituted phenyl only when R⁵ is tetrazol-5-yl, and R² is unsubstituted phenyl only when R⁴ is not hydroxy; or
R² and R³ form, together with the carbon atoms to which they are attached, a benzene ring optionally substituted with at least one R⁶, provided that said benzene ring is unsubstituted only when R⁸ is tetrazolyl or oxazolyl; or a 5- or 6-membered heteroaromatic or heterocyclic ring, optionally substituted with at least one R⁶;
L is 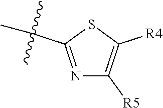
wherein R⁴ is COOR¹²; and R⁵ is selected from H and C1-C6 alkyl; or
R⁴ is selected from H and C1-C6 alkyl; and R⁸ is COOR¹²; or
(b) 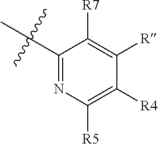
wherein R⁴ is selected from H and C1-C6 alkyl; R⁵ is selected from H and C1-C6 alkyl; and R″ is selected from C0-C1 alkyl-COOR¹²; or
R⁵ is selected from COOR¹²; and R″ is selected from H and C1-C6 alkyl; and
R is selected from H, C1-C6 alkyl, and nitro;
(c) 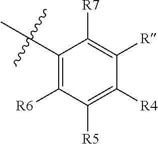
wherein R⁴ is selected from H, hydroxy and C1-C6 alkyl; R⁵ is selected from H, C1-C6 alkyl; and R″ is selected from C0-C1 alkyl-COOR¹²; or
R⁸ is selected from COOR¹², oxazol-5-yl and tetrazol-5-yl, said oxazol-5-yl and tetrazol-5-yl optionally being substituted by R⁹; and R″ is selected from H, C1-C6 alkyl, and nitro;
R⁷ is selected from H, C1-C6 alkyl, and nitro; and
R is selected from H, hydroxy, and C1-C6 alkyl; or
(d) 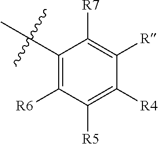
wherein R⁴ is selected from H and C1-C6 alkyl; and R⁵ is COOR¹²; R⁷ is selected from H, C1-C6 alkyl, and nitro; and R⁸ is selected from H, hydroxy, and C1-C6 alkyl;
provided that in any of (a), (b), (c) and (d), R⁴, R⁵ and R″ are selected from C0-C1 alkyl-COOR¹² only when at least one of R² or R³ is optionally substituted phenyl or optionally substituted heteroaryl; or when R² and R³ together with the carbon atoms to which they are attached form a benzene ring optionally substituted by at least one R⁶;
R⁶ is selected from C1-C6 alkyl, cyano, halogen, hydroxy, C1-C6 alkoxy, C1-C6 alkylthio, tetrahydropyrrolyl, R¹⁰R¹¹N, carbamoyl, and C1-C6 alkylcarbonylamino, or is an ethyleneoxy biradical forming, together with the atoms to which it is attached, a five-membered oxygen containing cycle; wherein any alkyl is optionally substituted with at least one halogen;
R⁹ is selected from C0-C1 alkyl-COOR¹²;
R¹⁰ and R¹¹ are independently selected from H and C1-C6 alkyl or form, together with the nitrogen to which they are attached, a 5- or 6-membered cyclic amino optionally containing one other cyclic heteroatom;
R¹² is selected from H, C1-C6 alkyl; heteroaryl-C0-C2 alkyl; (C1-C3 alkoxy)ₚC1-C3 alkyl; aryl-C0-C2 alkyl; heterocyclyl-C0-C2 alkyl; and C1-C6 dialkylamino-C1-C6 alkyl, wherein any cyclic moiety is optionally substituted with C1-C6 alkyl;
p is 1 or 2; or a pharmaceutically acceptable salt thereof;

30. The method of claim 17, wherein the small molecule is a compound of formula (X) 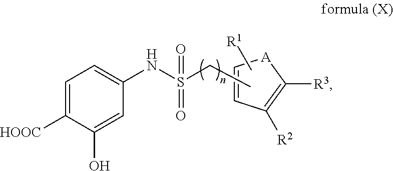
wherein: n is 0 or 1;
A is O, S, —CR⁴═CR⁴— or —CR⁴═N—;
R¹ is selected from H; halogen; C₁-C₆ alkyl, optionally substituted with at least one halogen; and C₁-C₆ alkoxy, substituted with at least one halogen;
R² and R³ are each independently selected from H; halogen; C₁-C₆ alkyl; C₁-C₆ alkoxy; secondary or tertiary C₁-C₆ alkylamido; carbocyclylcarbonylamino-C₁-C₂ alkyl; 5- or 6-membered cyclic aminocarbonyl; C₁-C₆ alkylcarbonylamino; C₁-C₆ alkylsulfonyl; hydroxy-C₀-C₆ alkyl, C₁-C₆ alkylcarbonyl; carboxy; C₁-C₆ alkoxycarbonyl; cyano; nitro; carbocyclyloxy; heterocyclyloxy; carbocyclyl-C₀-C₃ alkyl; carbocyclyl-C₂-C₃ alkenyl; heterocyclyl-C₀-C₃ alkyl; and heterocyclyl-C₂-C₃ alkenyl;
wherein any alkyl is optionally substituted with at least one halogen; any carbocyclyl or heterocyclyl is 5- or 6-membered monocyclyl or 9- or 10-membered bicyclyl; and any carbocyclyl or heterocyclyl is optionally substituted with at least one R⁵; or R² and R³ form, together with the carbon atoms to which they are attached, a 5- or 6-membered carbocyclic or heterocyclic ring, which ring is optionally substituted with at least one R⁵;
each R⁴ is independently selected from H, halogen, monocyclic C₃-C₆ carbocyclyl and C₁-C₆ alkyl, wherein any alkyl is optionally substituted with at least one halogen;
each R⁸ is independently selected from halogen; C₁-C₆ alkyl; C₁-C₆ alkoxy; phenoxy; amino; cyano; nitro; secondary or tertiary C₁-C₆ alkylamino; 5- or 6-membered cyclic amino; C₁-C₆ alkylcarbonylamino; carbamoyl; secondary or tertiary C₁-C₆ alkylamido; 5- or 6-membered cyclic aminocarbonyl; C₁-C₆ alkoxycarbonylamino; hydroxy-C₀-C₆ alkyl; C₁-C₆-alkylthio; carboxy-C₀-C₆-alkyl; C₁-C₆ alkoxycarbonyl; C₁-C₆ alkylcarbonyl; C₁-C₆-alkylsulfonyl; and C₁-C₆ alkylsulfonylamino; wherein any alkyl is optionally substituted with at least one halogen; or a pharmaceutically acceptable salt thereof, for use as a medicament, with the proviso that when A is CR⁴═CR⁴ and n is 0, then neither R² nor R³ is selected from 4-hydroxypyrazolo[1,5-a]-1,3,5-triazin-8-yl and 2,4-dihydroxypyrazolo[1,5-a]-1,3,5-triazin-8-yl; the compound is not selected from
4-(3,4-dichlorophenylsulfonamido)-2-hydroxybenzoic acid,
4-(2,5-dichlorophenylsulfonamido)-2-hydroxybenzoic acid,
4-(2,5-diethylphenylsulfonamido)-2-hydroxybenzoic acid,
4-(4-bromophenylsulfonamido)-2-hydroxybenzoic acid,
4-(3-carboxy-4-hydroxyphenylsulfonamido)-2-hydroxybenzoic acid,
2-hydroxy-4-(4-methylphenylsulfonamido)benzoic acid,
2-hydroxy-4-(phenylsulfonamido)benzoic acid, and
4-(4-ethylphenylsulfonamido)-2-hydroxybenzoic acid.

31. The method of claim 17, wherein the small molecule is a compound of formula (X) wherein: n is 0 or 1;
A is O, S, —CR⁴═CR⁴— or —CR⁴═N—;
R¹ is selected from H; halogen; C₁-C₆ alkyl, optionally substituted with at least one halogen; and C₁-C₆ alkoxy, substituted with at least one halogen;
R² and R³ are each independently selected from H; halogen; C₁-C₆ alkyl; C₁-C₆ alkoxy; secondary or tertiary C₁-C₆ alkylamido; carbocyclylcarbonylamino-C₁-C₂ alkyl; 5- or 6-membered cyclic aminocarbonyl; C₁-C₆ alkylcarbonylamino; C₁-C₆ alkylsulfonyl; hydroxy-C₀-C₆ alkyl, C₁-C₆ alkylcarbonyl; carboxy; C₁-C₆ alkoxycarbonyl; cyano; nitro; carbocyclyloxy; heterocyclyloxy; carbocyclyl-C₀-C₃ alkyl; carbocyclyl-C₂-C₃ alkenyl; heterocyclyl-C₀-C₃ alkyl; and heterocyclyl-C₂-C₃ alkenyl;
wherein any alkyl is optionally substituted with at least one halogen; any carbocyclyl or heterocyclyl is 5- or 6-membered monocyclyl or 9- or 10-membered bicyclyl; and any carbocyclyl or heterocyclyl is optionally substituted with at least one R⁵; or R² and R³ form, together with the carbon atoms to which they are attached, a 5- or 6-membered carbocyclic or heterocyclic ring, which ring is optionally substituted with at least one R⁵;
each R⁴ is independently selected from H, halogen, monocyclic C₃-C₆ carbocyclyl and C₁-C₆ alkyl, wherein any alkyl is optionally substituted with at least one halogen;
each R⁵ is independently selected from halogen; C₁-C₆ alkyl; C₁-C₆ alkoxy; phenoxy; amino; cyano; nitro; secondary or tertiary C₁-C₆ alkylamino; 5- or 6-membered cyclic amino; C₁-C₆ alkylcarbonylamino; carbamoyl; secondary or tertiary C₁-C₆ alkylamido; 5- or 6-membered cyclic aminocarbonyl; C₁-C₆ alkoxycarbonylamino; hydroxy-C₀-C₆ alkyl; C₁-C₆-alkylthio; carboxy-C₀-C₆-alkyl; C₁-C₆ alkoxycarbonyl; C₁-C₆ alkylcarbonyl; C₁-C₆-alkylsulfonyl; and C₁-C₆ alkylsulfonylamino; wherein any alkyl is optionally substituted with at least one halogen; or a pharmaceutically acceptable salt thereof, for use as a medicament, with the proviso that when A is CR⁴═CR⁴ and n is 0, then neither R² nor R³ is selected from 4-hydroxypyrazolo[1,5-a]-1,3,5-triazin-8-yl and 2,4-dihydroxypyrazolo[1,5-a]-1,3,5-triazin-8-yl.

32. The method of claim 17, wherein the small molecule is a compound of formula (XI) 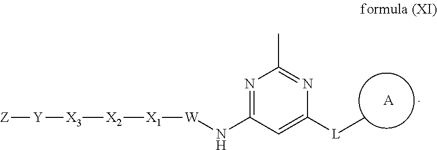
or a pharmaceutically acceptable salt thereof, wherein: W is a branched or straight C₁₋₁₂ aliphatic chain wherein up to two carbon units are optionally and independently replaced by —C(Q₁)₂-, —C(Q₂)₂-, -CHQ₁-, —CHQ₂-, —CO—, —CS—, —CONR^{A}—, —CONR^{A}NR^{A}—, —CO₂—, —OCO—, —NR^{A}—, —NR^{A}CO₂—, —O—, —NR^{A}CONR^{A}—, —OCONR^{A}—, —NR^{A}NR^{A}—, —NR^{A}CO—, —S—, —SO—, —SO₂—, —SO₂NR^{A}—, —NR^{A}SO₂—, or —NR^{A}SO₂NR^{A};
each R^{A} is independently hydrogen C₁₋₈ aliphatic; cycloaliphatic, heterocycloaliphatic, aryl, or heteroaryl, optionally substituted with 1-3 of Q₁ or Q₂;
X₁, X₂, and X₃ are each independently absent or are a cycloaliphatic, heterocycloaliphatic, aryl, or heteroaryl, each or which are optionally and independently substituted with 1-3 of Q₁, or Q₂, and wherein at least one of X₁, X₂ and X₃ is present;
Y is absent or is a branched or straight C₁₋₁₂ aliphatic chain wherein up to two carbon units are optionally and independently replaced by —C(Q₁)₂-, —C(Q₂)₂-, —CHQ₁-, —CHQ₂-, —CO—, —CS—, —CONR^{B}—, —C(═NR^{B})NR^{B}—, —C(═NOR^{B})NR^{B}—, —NR^{B}C(═NR^{B})NR^{B}—, —CONR^{B}NR^{B}—, —CO₂—, —OCO—, —NR—, —NR^{B}CO₂—, —O—, —NR^{B}CONR^{B}—, —OCONR^{B}—, —NR^{B}NR^{B}—, —NR^{B}CO—, —S—, —SO—, —SO₂—, —SO₂NR^{B}—, —NRSO₂—, or —NR^{B}SO₂NR;
each R^{B} is independently hydrogen, C₁₋₈ aliphatic, cycloaliphatic, heterocycloaliphatic, aryl, or heteroaryl, optionally substituted with 1-3 of Q₁ or Q₂;
Z is independently hydrogen, C₁₋₈ aliphatic, cycloaliphatic, heterocycloaliphatic, aryl, or heteroaryl, optionally substituted with 1-3 of Q₁ or Q₂; or
L is absent or is NH, N(C₁₋₈ aliphatic), or is a branched or straight C aliphatic chain wherein up to two carbon units of L are optionally and independently replaced by —C(Q₁)₂-, —C(Q₂)₂-, —CO—, —CS—, —CONR_{C}—, —CONR^{C}NR^{C}—, —CO₂—, —OCO—, —NR^{C}—, —NR^{C}CO₂—, —O—, —NR^{C}CONR^{C}—, —OCONR^{C}—, —NR^{C}NR^{C}—, —NR^{C}CO—, —S—, —SO—, —SO₂—, —SO₂NR^{C}—, —NR^{C}SO₂—, or —NR^{C}SO₂NR^{C};
each R^{C} is independently hydrogen, C₁₋₈ aliphatic, cycloaliphatic, heterocycloaliphatic, aryl, or heteroaryl, optionally substituted with 1-3 of Q₁ or Q₂;
Ring A is a monocyclic, bicyclic, or tricyclic cycloaliphatic, heterocycloaliphatic, aryl, or heteroaryl, any of which may be optionally substituted with 1-3 of halo, —OH, oxo, —CF₃, —OCF₃, cyano, or a C₁₋₈ branched or straight aliphatic, wherein 1-3 methylene groups of the aliphatic are optionally and independently replaced with —C(O)—, —O—, —NH—, —C(O)NH—, or —C(O)O—, and wherein the aliphatic is optionally further substituted with 1-3 of halo, cyano, OH or C₁₋₃ aliphatic;
each Q, is independently halo, oxo, —CN, —NO₂, —N═O, —NHOQ₂, =NQ₂, =NOQ₂, —OQ₂, —SOQ₂, —SO₂Q₂, —SON(Q₂)₂, —SO₂(Q₂)₂, —N(Q₂)₂, —C(O)OQ₂, —C(O)-Q₂, —C(O)N(Q₂)₂, —C(═NQ₂)NQ₂-, —NQ₂C(═NQ₂)NQ₂-, —C(O)N(Q₂)(OQ₂), —N(Q₂)C(O)-Q₂, —N(Q₂)C(O)N(Q₂)₂, —N(Q₂)C(O)O-Q₂, —N(Q₂)SO₂-Q₂, —N(Q₂)SO-Q₂ or aliphatic optionally including 1-3 substituents independently selected from Q₂ or Q₃,
each Q₂ is independently hydrogen, aliphatic, alkoxy, cycloaliphatic, aryl, arylalkyl, heterocyclic, or heteroaryl ring, each optionally including 1-3 substituents independently selected from Q₃;
each Q₃ is halo, oxo, CN, NO₂, NH₂, CF₃OCF₃, OH, —COOH, or C₁-C₄ alkyl optionally substituted with 1-3 of halo, oxo, —CN, —NO₂, —CF₃, —OCF₃, —OH, —SH, —S(O)₃H, —NH₂, or —COOH;
provided that the compound of formula XI is not

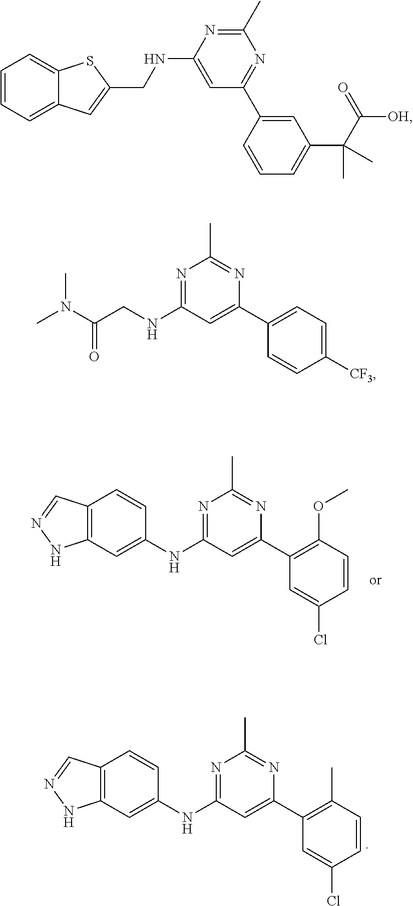 [SMILES: Cc1nc(NCc2cc3ccccc3s2)cc(-c2cccc(C(C)(C)C(=O)O)c2)n1 ; Cc1nc(NCC(=O)N(C)C)cc(-c2ccc(C(F)(F)F)cc2)n1 ; COc1ccc(Cl)cc1-c1cc(Nc2ccc3cn[nH]c3c2)nc(C)n1 ; Cc1nc(Nc2ccc3cn[nH]c3c2)cc(-c2cc(Cl)ccc2C)n1]

33. The method of claim 17, wherein the small molecule is a compound of formula (XI) or a pharmaceutically acceptable salt thereof, wherein:
W is a branched or straight C₁₋₁₂ aliphatic chain wherein up to two carbon units are optionally and independently replaced by —C(Q₁)₂-, —C(Q₂)₂-, —CHQ₁-, —CHQ₂-, —CO—, —CS—, —CONR^{A}—, —CONR^{A}NR^{A}—, —CO₂—, —OCO—, —NR^{A}—, —NR^{A}CO₂—, —O—, —NR^{A}CONR^{A}—, —OCONR^{A}—, —NR^{A}NR^{A}—, —NR^{A}CO—, —S—, —SO—, —SO₂—, —SO₂NR^{A}—, —NR^{A}SO₂—, or —NR^{A}SO₂NR^{A};
each R^{A} is independently hydrogen, C₁₋₈ aliphatic, cycloaliphatic, heterocycloaliphatic, aryl, or heteroaryl, optionally substituted with 1-3 of Q₁ or Q₂;
X₁, X₂, and X₃ are each independently absent or are a cycloaliphatic, heterocycloaliphatic, aryl, or heteroaryl, each or which are optionally and independently substituted with 1-3 of Qi, or Q₂, and wherein at least one of X₁, X₂ and X₃ is present;
Y is absent or is a branched or straight C₁₋₁₂ aliphatic chain wherein up to two carbon units are optionally and independently replaced by —C(Q₁)₂-, —C(Q₂)₂-, —CHQ₁-, —CHQ₂-, —CO—, —CS—, —CONR^{B}—, —C(═NR^{B})NR^{B}—, —C(═NOR^{B})NR^{B}—, —NR^{B}C(═NR^{B})NR^{B}—, —CONR^{B}NR^{B}—, —CO₂—, —OCO—, —NR—, —NR^{B}CO₂—, —O—, —NR^{B}CONR^{B}—, —OCONR^{B}—, —NR^{B}NR^{B}—, —NR^{B}CO—, —S—, —SO—, —SO₂—, —SO₂NR^{B}—, —NRSO₂—, or —NR^{B}SO₂NR;
each R^{B} is independently hydrogen, C₁₋₈ aliphatic, cycloaliphatic, heterocycloaliphatic, aryl, or heteroaryl, optionally substituted with 1-3 of Q₁ or Q₂;
Z is independently hydrogen, C₁₋₈ aliphatic, cycloaliphatic, heterocycloaliphatic, aryl, or heteroaryl, optionally substituted with 1-3 of Q₁ or Q₂; or
L is absent or is NH, N(C₁₋₈ aliphatic), or is a branched or straight C aliphatic chain wherein up to two carbon units of L are optionally and independently replaced by —C(Q₁)₂-, —C(Q₂)₂-, —CO—, —CS—, —CONR^{C}—, —CONR^{C}NR^{C}—, —CO₂—, —OCO—, —NR^{C}—, —NR^{C}CO₂—, —O—, —NR^{C}CONR^{C}—, —OCONR^{C}—, —NR^{C}NR^{C}—, —NR^{C}CO—, —S—, —SO—, —SO₂—, —SO₂NR^{C}—, —NR^{C}SO₂—, or —NR^{C}SO₂NR^{C};
each R^{C} is independently hydrogen, C₁₋₈ aliphatic, cycloaliphatic, heterocycloaliphatic, aryl, or heteroaryl, optionally substituted with 1-3 of Q₁ or Q₂;
Ring A is a monocyclic, bicyclic, or tricyclic cycloaliphatic, heterocycloaliphatic, aryl, or heteroaryl, any of which may be optionally substituted with 1-3 of halo, —OH, oxo, —CF₃, —OCF₃, cyano, or a C₁₋₈ branched or straight aliphatic, wherein 1-3 methylene groups of the aliphatic are optionally and independently replaced with —C(O)—, —O—, —NH—, —C(O)NH—, or —C(O)O—, and wherein the aliphatic is optionally further substituted with 1-3 of halo, cyano, OH or C₁₋₃ aliphatic;
each Q, is independently halo, oxo, —CN, —NO₂, —N═O, —NHOQ₂, =NQ₂, =NOQ₂, —OQ₂, —SOQ₂, —SO₂Q₂, —SON(Q₂)₂, —SO₂ (Q₂)₂, —N(Q₂)₂, —C(O)OQ₂, —C(O)-Q₂, —C(O)N(Q₂)₂, —C(═NQ₂)NQ₂-, —NQ₂C(═NQ₂)NQ₂-, —C(O)N(Q₂)(OQ₂), —N(Q₂)C(O)-Q₂, —N(Q₂)C(O)N(Q₂)₂, —N(Q₂)C(O)O-Q₂, —N(Q₂)SO₂-Q₂-N(Q₂)SO-Q₂ or aliphatic optionally including 1-3 substituents independently selected from Q₂ or Q₃;
each Q₂ is independently hydrogen, aliphatic, alkoxy, cycloaliphatic, aryl, arylalkyl, heterocyclic, or heteroaryl ring, each optionally including 1-3 substituents independently selected from Q₃;
each Q₃ is halo, oxo, CN, NO₂, NH₂, CF₃OCF₃, OH, —COOH, or C₁-C₄ alkyl optionally substituted with 1-3 of halo, oxo, —CN, —NO₂, —CF₃, —OCF₃, —OH, —SH, —S(O)₃H, —NH₂, or —COOH;

34. The method of claim 17, wherein the small molecule is a compound of formula (XII) 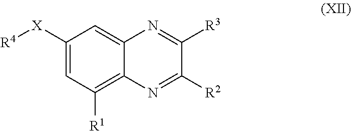
wherein
X denotes N—R⁵ or O;
R¹ denotes ArX, ArX—Ar^{Y}, Ar^{X}-Hetar^{Y}, Ar-Hetcyc^{Y}, Ar^{X}-LA^{Z}-Ar^{Y}, ArX-LA^{Z}-Hetar^{Y}, Ar^{X}-LAZ-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, CA^{X};
R² and R¹ denote independently from each other H, —OH, —SH, straight-chain or branched —C₁₋₆-alkyl, straight-chain or branched —C₂₋₆-alkenyl, straight-chain or branched—O—C₁₋₆-alkyl, straight-chain or branched —S-C₁₋₆-alkyl, Hal, —CN, —NH₂, —NH(C₁₋₄-alkyl), N(C₁₋₄-alkyl)₂, wherein the C₁₋₄-alkyl substituents may be the same or different and may be straight-chain or branched;
R⁴ denotes Ar^{W} or Hetar^{W}; Ar^{W} or Hetar^{W} bears in its ortho-position (relative to the attachment of R⁴ to X) one (1) substituent R^{W1} and may or may not bear further substituents;
R⁵ denotes H, ArX, Hetar^{X}, Hetcyc^{X}, LA^{X}, CA^{X};
Ar^{W} denotes a mono-, bi- or tricyclic aromatic ring system with 5, 6, 7, 8, 9, 10, 11, 12, 13, 14 ring carbon atoms; the ring system may bear—besides the ortho-substituent R^{W1}—no further substituent or one (1) further substituent R^{W2} or two (2) further substituents R^{W2}, R^{W3}, that may be the same or different;
Ar^{X} denotes a mono-, bi- or tricyclic aromatic ring system with 5, 6, 7, 8, 9, 10, 11, 12, 13, 14 ring carbon atoms, wherein the ring system may be unsubstituted or mono-, di- or trisubstituted with independently from each other R^{X1}, R^{X2}, R^{X3};
Ar^{Y} denotes a mono-, bi- or tricyclic aromatic ring system with 5, 6, 7, 8, 9, 10, 11, 12, 13, 14 ring carbon atoms, wherein the ring system may be unsubstituted or mono-, di- or trisubstituted with independently from each other R^{Y1}, R^{Y2}, R^{Y3};
Hetar^{W} denotes a mono-, bi- or tricyclic aromatic ring system with 5, 6, 7, 8, 9, 10, 11, 12, 13, 14 ring atoms, wherein 1, 2, 3, 4, 5 of said ring atoms is/are a hetero atom(s) selected from N, O and/or S, and the remaining are carbon atoms, wherein that ring system may bear—besides the ortho-substituent R^{W1}—no further substituent or one (1) further substituent R^{W2} or two (2) further substituents R^{W2}, R^{W3} that may be the same or different;
Hetar^{X} denotes a mono-, bi- or tricyclic aromatic ring system with 5, 6, 7, 8, 9, 10, 11, 12, 13, 14 ring atoms, wherein 1, 2, 3, 4, 5 of said ring atoms is/are a hetero atom(s) selected from N, O and/or S, and the remaining are carbon atoms, wherein that aromatic ring system may be unsubstituted or mono-, di- or tri-substituted with independently from each other R^{X1}, R^{X2}, R³;
Hetar^{Y} denotes a mono-, bi- or tricyclic aromatic ring system with 5, 6, 7, 8, 9, 10, 11, 12, 13, 14 ring atoms, wherein 1, 2, 3, 4, 5 of said ring atoms is/are a hetero atom(s) selected from N, O and/or S, and the remaining are carbon atoms, wherein that aromatic ring system may be unsubstituted or mono-, di- or tri-substituted with independently from each other R^{Y1}, R^{Y2}, R^{Y3};
Hetcyc^{X} denotes a saturated or partially unsaturated mono-, bi- or tricyclic heterocycle with 3, 4, 5, 6, 7, 8, 9, 10, 11, 12, 13, 14 ring atoms, wherein 1, 2, 3, 4, 5 ring atom(s) is/are heteroatom(s) selected from N, O and/or S, and the remaining ring atoms are carbon atoms, wherein that heterocycle may be unsubstituted or mono-, di- or trisubstituted with R^{X4}, R^{X5}, R^{X6};
Hetcyc^{Y} denotes a saturated or partially unsaturated mono-, bi- or tricyclic heterocycle with 3, 4, 5, 6, 7, 8, 9, 10, 11, 12, 13, 14 ring atoms, wherein 1, 2, 3, 4, 5 ring atom(s) is/are heteroatom(s) selected from N, O and/or S, and the remaining ring atoms are carbon atoms, wherein that heterocycle may be unsubstituted or mono-, di- or trisubstituted with R^{Y4}, R^{Y5}, R^{Y6};
R^{W1} denotes Hal, LA^{X}, CA^{X}, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{X}, Hetar^{X}-LA^{Z}-Ar^{Y}, Heta^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, —CN, —NO₂, —SO₂NH₂, —SO₂NHR^{W4}, —SO₂NR^{W4}R^{W5}, —NH—SO₂—R^{W6}, —NR^{W4}—SO₂—R^{W6}, —S—R^{W6}, —S(═O)—R^{W6}, —SO₂—R^{W6}, —NH₂, —NHR^{W4}, —NR^{W4}R^{W5}, —OH, —O—R^{W6}, —CHO, —C(═O)—R^{W6}, —COOH, —C(═O)—O—R^{W6}, —C(═O)—NH₂, —C(═O)—NHR^{W4}, —C(═O)—NR^{W4}R^{W5}, —NH—C(═O)—R^{W6}, —NR^{W4}—C(═O)—R^{W6}, —NH—(C₁₋₃-alkylene)-C(═O)—NH₂, —NH—(C₁₋₃-alkylene)-C(═O)—NHR^{W4}, —NH—(C₁₋₃-alkylene)-C(═O)—NR^{W4}R^{W5}, or
R^{W1} and R⁵ form together a divalent alkylene chain with 1, 2, 3, 4, 5 chain carbon atoms, wherein 2 adjacent CH₂ groups may together be replaced by a —CH═CH— moiety; the divalent alkylene chain may be straight-chain or branched and may be unsubstituted or mono- or di-substituted with independently from each other straight-chain or branched —C₁₋₆-alkyl or ═O (oxo);
R^{W2}, R^{W3} denote independently from each other H, Hal, LA^{X}, CA^{X}, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, —CN, —NO₂, —SO₂NH₂, —SO₂NHR^{W4}, —SO₂NR^{W4}R^{W5}, —NH—SO₂—R^{W6}, —NR^{W4}—SO₂—R^{W6}, —S—R^{W6}, —S(═O)—R^{W6}, —SO₂—R^{W6}, —NH₂, —NHR^{W4}, —NR^{W4}R^{W5}—NH—C(═O)—R^{W6}, —NR^{W4}—C(═O)—R^{W6}, —OH, —O—R^{W6}, —CHO, —C(═O)—R^{W6}, —COOH, —C(═O)—O—R^{W6}, —C(═O)—NH₂, —C(═O)—NHR^{W4}, —C(═O)—NR^{W4}R^{W5}, —C(═O)—NH—NH₂, —C(═O)—NH—NHR^{W4}, —NH—(C₁₋₃-alkylene)-C(═O)—NH₂, —NH—(C₁₋₃-alkylene)-C(═O)—NHR^{W4}, —NH—(C₁₋₃-alkylene)-C(═O)—NR^{W4}R^{W5},
or
two of R^{W1}, R^{W2} and R^{W3} form a divalent alkylene chain with 3, 4, 5 chain carbon atoms, wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, wherein the divalent alkylene chain may be unsubstituted or mono- or di-substituted with independently from each other straight-chain or branched —C₁₋₆-alkyl or ═O (oxo);
R^{X1}, R^{X2}, R^{X3} denote independently from each other H, Hal, LA^{X}, CA^{X}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9},—NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}
or
two of R^{X1}, R^{X2}, R^{X3} form a divalent alkylene chain with 3, 4, 5 chain carbon atoms, wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, wherein the divalent alkylene chain may be unsubstituted or mono- or di-substituted with independently from each other straight-chain or branched —C₁₋₆-alkyl or ═O (oxo);
R^{X4}, R^{X5}, R^{X6} denote independently from each other H, Hal, LA^{X}, CA^{X}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═)-NR^{X7}R^{X8}, oxo (═O);
R^{Y1}, R^{Y2}, R^{Y3} denote independently from each other H, Hal, LA^{Y}, CA^{Y}, —CN, —NO₂, —SF₅, —SO₂NH₂, —SO₂NHR^{Y7}, —SO₂NR^{Y7}R^{Y8}, —NH—SO₂—R^{Y9}, —NR⁷—SO₂—R^{Y9}, —S—R^{Y9}, —S(═O)—R^{Y9}, —SO₂—R^{Y9}, —NH₂, —NHR^{Y7}, —NR ⁷R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR⁷—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}
or
two of R^{Y1}, R^{Y2}, R^{Y3} form a divalent alkylene chain with 3, 4, 5 chain carbon atoms, wherein 1 or 2 non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, wherein the divalent alkylene chain may be unsubstituted or mono- or di-substituted with independently from each other straight-chain or branched —C₁₋₆-alkyl or ═O (oxo);
R^{Y4}, R^{Y5}, R^{Y6} denote independently from each other H, Hal, LA^{Y}, CA^{Y}, —CN, —NO₂, —SF₅, —SO₂NH₂, —SO₂NHR^{Y7}, —SO₂NR^{Y7}R^{Y8}, —NH—SO₂—R^{Y9}, —NR⁷—SO₂—R^{Y9}, —S—R^{Y9}, —S(═O)—R^{Y9}, —SO₂—R^{Y9}, —NH₂, —NHR^{Y7}, —NR ⁷R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR ⁷R^{Y8}, —NH-C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, or ═O (oxo);
LA^{X} denotes straight-chain or branched C₁₆-alkyl which may be unsubstituted or mono-, di- or trisubstituted with independently from each other Hal, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)-R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O), wherein 1 or 2 non-adjacent CH₂ groups of the C₁₋₆-alkyl radical may independently from each other be replaced by O, S, N(H) or N-R^{X7}, and/or 1 or 2 non-adjacent CH groups of the C₁₋₆-alkyl radical may independently from each other be replaced by N;
LA^{Y} denotes straight-chain or branched C₁₋₆-alkyl which may be unsubstituted or mono-, di- or trisubstituted with independently from each other Hal, —CN, —NO₂, —SF₅, —SO₂NH₂, —SO₂NHR^{Y7}, —SO₂NR^{Y7}R^{Y8}, —NH—SO₂—R^{Y9}, —NR⁷—SO₂—R^{Y9}, —S—R^{Y9}, —S(═O)—R^{Y9}, —SO₂—R^{Y9}, —NH₂, —NHR^{Y7}, —NR^{Y}R⁸, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, oxo (═O), wherein 1 or 2 non-adjacent CH₂ groups of the C₁₋₆-alkyl radical may independently from each other be replaced by O, S, N(H) or N—R^{Y7} and/or 1 or 2 non-adjacent CH groups of the C₁₋₆-alkyl radical may independently from each other be replaced by N;
LA^{Z} denotes a divalent straight-chain or branched C₁₋₆-alkylene radical, wherein the alkylene radical may be unsubstituted or mono-, di- or trisubstituted with independently from each other Hal, —CN, —NO₂, —SF₅, —SO₂NH₂, —SO₂NHR^{Z7}, —SO₂NR^{Z7}R^{Z8}, —NH—SO₂—R^{Z9}, —NR^{Z7}—SO₂—R^{Z9}, —S—R^{Z9}, —S(═O)—R^{Z9}, —SO₂—R^{Z9}, —NH₂, —NHR^{Z7}, —NR^{Z7}R^{Z8}, —OH, —O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—C(═O)—R^{Z9}, —NR^{Z7}—C(═O)—R^{Z9}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, oxo (═O), wherein 1 or 2 non-adjacent CH₂ groups of that divalent alkylene radical may be replaced independently from each other by O, S, —N(H) or N—R^{Z7}, and/or 1 or 2 non-adjacent CH groups of that divalent alkylene radical may be replaced by N;
R^{W4}, R^{W6}, R^{W6} denote Ar^{X}, Ar^{X}—Ar^{Y}, ArX-Hetar^{Y}, Ar^{X}-Hetcyc^{Y}, Ar^{X}-LA^{Z}-Ar^{Y}, Ar^{X}—Ar^{Z}-Hetar^{Y}, ArX-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{Z}-LA^{Z}-Hetcyc^{Y}, LA^{X}, LA^{Z}-Ar^{Y}, LA^{Z}-Hetar^{Y}, LA^{Z}-Hetcyc^{Y}, CA^{X}
or
R^{W4} and R^{W5} form together with the nitrogen atom, which they are attached to, a 3-, 4-, 5-, 6- or 7-membered heterocycle, wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7}, R^{X8}, R^{X9}, R^{Y7}, R^{Y8}, R^{Y9}, R^{Z7}, R^{Z8}, R^{Z9} denote independently from each other straight-chain or branched C₁₋₆-alkyl, which may be unsubstituted or mono-, di- or trisubstituted with independently from each other Hal, —CN, —NO₂, —SF₅, —SO₂NH₂, —SO₂NHR^{X7v}, —SO₂NR^{X7v}R^{X8v}, —NH—SO₂—R^{X9v}, NR^{X7v}—SO₂—R^{X9v}, —S—R^{X9v}, —S(═O)—R^{X9v}, SO₂-R^{X9v}—NH₂, —NHR^{X7v}, —NR^{X7v}R^{X8v}, —OH, —O—R^{X9v}, —CHO, —C(═O)—R^{X9v}, —COOH, —C(═O)—O—R^{X9v}, —C(═O)—NH₂, —C(═O)—NHR^{X7v}, —C(═O)—NR^{X7v}R^{X8v}, —NH-C(═O)—R^{X9v}, —NR^{X7v}—C(═O)—R^{X9v}, —NH—(C₁₋₃-alkylene)-C(═O)—NH₂, —NH—(C₁₋₃-alkylene)-C(═O)—NHR^{X7v}, —NH—(C₁₋₃-alkylene)-C(═O)—NR^{X7v}R^{X8v}, oxo (═O), wherein 1 or 2 non-adjacent CH₂ groups of the C₁₋₆-alkyl radical may independently from each other be replaced by O, S, N(H) or N-R^{X7v}, and/or 1 or 2 non-adjacent CH groups of the C₁₋₆-alkyl radical may independently from each other be replaced by N, or a saturated monocyclic carbocycle with 3, 4, 5, 6, 7 carbon atoms, which may be unsubstituted or mono- or disubstituted with independently from each other Hal, ArX, A^{X}-A^{Y}, Ar^{X}-Hetar^{Y}, Ar^{X}-Hetcyc^{Y}, Ar^{X}-LA^{X}-Ar^{Y}, Ar^{X}-LA^{Z}-Hetar^{Y}, Ar^{X}-LA^{Z}-Hetcyc^{Y}, Hetar^{X}, Hetar^{X}-A^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SF₅, —SO₂NH₂, —SO₂NHR^{X7v}, —SO₂NR^{X7v}R^{X8v}, —NH—SO₂—R^{X9v}, —NR^{X7v}—SO₂—R^{X9v}, —S-R^{X9v}S(═)-R^{X9v}, —SO₂—R^{X9v}, —NH₂, —NHR^{X7}V, —NR^{X7v}R^{X8v}, —OH, —O—R^{X9}, —CHO, —C(═O)—R^{X9v}, —COOH, —C(═O)—O—R^{X9v}, —C(═O)—NH₂, —C(═O)—NHR^{X7v}, —C(═O)—NR^{X7v}R^{X8v}, —NH—C(═O)—R^{X9v}, —NR^{X7v}—C(═O)—R^{X9v}, —NH—(C₁₋₃-alkylene)-C(═O)—NH₂, —NH—(C₁₋₃-alkylene)-C(═O)—NHR^{X7v}, —NH—(C₁₋₃-alkylene)-C(═O)—NR^{X7v}R^{X8v}, oxo (═O), with the proviso that if any of the substituents of that monocyclic carbocycle is ArX, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, HetarX-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{Z}-LA^{Z}-Hetcyc^{Y}, LA^{X}, LA^{Z}-Ar^{Y}, LA^{Z}-Hetar^{Y}, LA^{Z}-Hetcyc^{Y}, then any radical R^{X7}, R^{X8}, R^{X9}, R^{Z7}, R^{Y8}, R^{Y9}, R^{Z7}, R^{Z8}, R^{Z9} of any substituent of ArX, Ar^{Y}, Hetar^{X}, Hetar^{Y}, Hetcyc^{X}, Hetcyc^{Y}, LA^{X} and LA^{Z} may not denote a mono- or disubstituted monocyclic carbocycle, or a saturated monocyclic heterocycle with 3, 4, 5, 6, 7 ring atoms, wherein 1 or 2 ring atom(s) is/are heteroatom(s) selected from N, O and/or S, and the remaining ring atoms are carbon atoms, wherein that heterocycle may be unsubstituted or substituted with straight-chain or branched C₁₋₆-alkyl, —C(═O)—C₁₋₆-alkyl (straight-chain or branched) and/or oxo (═O), or a phenyl, —CH₂-phenyl, -naphthyl, —CH₂-naphthyl, heteroaromatic ring system or —CH₂-heteroaromatic ring system with 5, 6, 7, 8, 9, 10, 11 ring atoms, wherein 1, 2, 3, 4, 5 of said ring atoms of said heteroaromatic ring system is/are hetero atom(s) selected from N, O and/or S, and the remaining are carbon atoms, wherein said phenyl, naphthyl or heteroaromatic ring system may be unsubstituted or mono-, di- or trisubstituted with independently from each other straight-chain or branched C₁₋₆-alkyl or —O—C₁₋₆-alkyl, Hal or —C(═O)—C₁₋₆-alkyl (straight-chain or branched);
or
each pair R^{X7} and R^{X8}; R^{Y7} and R^{Y8}; R^{Z7} and R^{Z8} form together with the nitrogen atom, which they are attached to, a 3-, 4-, 5-, 6- or 7-membered heterocycle, wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7v}, R^{X8v}, R^{X9v} denotes independently from each other straight-chain or branched C₁₋₆-alkyl, which may be unsubstituted or mono-, di- or trisubstituted with Hal, or a unsubstituted saturated monocyclic carbocycle with 3, 4, 5, 6, 7 carbon atoms;
or
R^{X7v} and R^{X8v} form together with the nitrogen atom, which they are attached to, a 3-, 4-, 5-, 6- or 7-membered heterocycle, wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
CA^{X}, CA^{Y} denote independently from each other a saturated monocyclic carbocycle with 3, 4, 5, 6, 7 carbon atoms, wherein the carbocycle may be unsubstituted or mono- or disubstituted with independently from each other R^{CA1}, R^{CA2};
R^{CA1}, R^{CA2} denote independently from each other H, Hal, ArX, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O), with the proviso that if R^{CA1} or R^{CA2} denotes ArX, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X} Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{Z}-Ar^{Y}, LA^{Z}-Hetar^{Y}, LA^{Z}-Hetcyc^{Y}, then ArX, Ar^{Y}, Hetar^{X}, Hetar^{Y}, Hetcyc^{X}, Hetcyc^{Y} may not be substituted with CA^{X} or CA^{Y};
Hal denotes F, Cl, Br, I;
or derivatives, N-oxides, prodrugs, solvates, tautomers or stereoisomers thereof as well as the physiologically acceptable salts of each of the foregoing, including mixtures thereof in all ratios.

35. The method of claim 17, wherein the small molecule is a compound of formula (XIII) 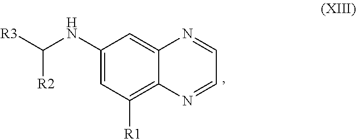
wherein R1 denotes N-methyl-indol-6-yl (1-methyl-1H-indol-6-yl), 3-methyl-1-benzofuran-5-yl, 1-methyl-iH-pyrrolo[3,2-b]pyridin-6-yl;
R2 denotes 1H-pyrazol-4-yl or 1-methyl-1H-pyrazol-4-yl and R3 denotes 1H-imidazol-2-yl, 1-methyl-1H-imidazol-2-yl, 1H-imidazol-5-yl, 1-methyl-1H-imidazol-5-yl, 1H-1,2,3-triazol-5-yl, 1-methyl-1H-1,2,3-triazol-5-yl, morpholin-2-yl, morpholin-3-yl, pyridin-3-yl, pyridin-4-yl, 4H-1,2,4-triazol-3-yl, 4-methyl-4H-1,2,4-triazol-3-yl;
or R2 denotes 1H-pyrazol-3-yl or 1-methyl-1H-pyrazol-3-yl and R3 denotes 1H-1,2,3-triazol-5-yl, 1-methyl-1H-1,2,3-triazol-5-yl, 4H-1,2,4-triazol-3-yl, 4-methyl-4H-1,2,4-triazol-3-yl;
or R2 denotes 1H-pyridazin-6-on-3-yl, 6-methoxypyridazin-3-yl and R3 denotes pyridin-3-yl, pyridin-4-yl;
or derivatives, N-oxides, prodrugs, solvates, tautomers or stereoisomers thereof as well as the physiologically acceptable salts of each of the foregoing, including mixtures thereof in all ratios.

36. The method of claim 17, wherein the small molecule is selected from from the group consisting of the following items:
Item 1 The compound of formula 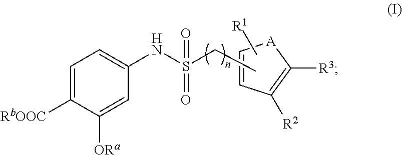
wherein n is 0 or 1; A is CR⁴═CR⁴—
R¹ is selected from H; halogen; C1-C6 alkyl, optionally substituted with at least one halogen; and C1-C6 alkoxy, optionally substituted with at least one halogen;
R² is selected from carbocyclyl-C0-C3 alkyl and heterocyclyl-C0-C3 alkyl; wherein any alkyl is optionally substituted with at least one halogen; any carbocyclyl or heterocyclyl is 5- or 6-membered monocyclyl or 9- or 10-membered bicyclyl; and any carbocyclyl or heterocyclyl is optionally substituted with at least one R⁵;
R³ is selected from H; halogen; C1-C6 alkyl; C1-C6 alkoxy; C1-C6 alkylcarbonylamino; hydroxy-C0-C6 alkyl, C1-C6 alkylcarbonyl; C1-C6 alkoxycarbonyl; and cyano; wherein any alkyl is optionally substituted with at least one halogen;
or R² and R³ form, together with the carbon atoms to which they are attached, a 5- or 6-membered carbocyclic or heterocyclic ring, which ring is optionally substituted with at least one R⁵;
each R⁴ is independently selected from H, halogen, monocyclic C3-C6 carbocyclyl and C1-C6 alkyl, wherein any alkyl is optionally substituted with at least one halogen;
each R⁵ is independently selected from halogen; C1-C6 alkyl; C1-C6 alkoxy; phenoxy; amino; cyano; nitro; secondary or tertiary C1-C6 alkylamino; 5- or 6-membered cyclic amino, optionally containing at least one further heteroatom in the ring; C1-C6 alkylcarbonylamino; carbamoyl; secondary or tertiary C1-C6 alkylamido; 5- or 6-membered cyclic aminocarbonyl; C1-C6 alkoxycarbonylamino; hydroxy-C0-C6 alkyl; C1-C6-alkylthio; carboxy-C0-C6-alkyl; C1-C6 alkoxycarbonyl; C1-C6 alkylcarbonyl; C1-C6-alkylsulfonyl; and C1-C6 alkylsulfonylamino; wherein any alkyl is optionally substituted with at least one halogen;
R^{a} is selected from H and C1-C6 alkylcarbonyl;
R^{b} is selected from H, C1-C6 alkyl, C1-C6 alkyl substituted with at least one R⁶; carbocyclyl-C0-C5 alkyl; and heterocyclyl-C0-C5 alkyl; wherein any carbocyclyl and heterocyclyl is 5- or 6-membered and is optionally substituted with at least one R⁷ and optionally comprises at least one oxo group in the ring;
provided that R^{a} and R^{b} are not both H;
each R⁶ is independently selected from hydroxy; C1-C6 alkoxy; hydroxy-C1-C6 alkoxy; C1-C6 alkylcarbonyloxy; C1-C6 alkoxycarbonyloxy; 5- or 6-membered carbocyclylcarbonyl or heterocyclylcarbonyl; amino; secondary or tertiary C1-C6 alkylamino; secondary or tertiary hydroxy-C1-C6 alkylamino; 5- or 6-membered cyclic amino optionally containing at least one further heteroatom in the ring and wherein the ring is optionally substituted with at least one C1-C6 alkyl; C1-C6 alkylcarbonylamino; C1-C6 alkoxycarbonylamino; (C1-C6 alkoxycarbonyl)(C1-C6 alkyl)amino; (C1-C6 alkoxycarbonyl)(5- or 6-membered carbocyclyl or heterocyclyl)amino; (C1-C6 alkylcarbonyl)(C1-C6 alkyl)amino; carbamoyl; secondary or tertiary C1-C6 alkylamido wherein any alkyl is optionally substituted by OH or CONH₂; 5- or 6-membered carbocyclyl- or heterocyclylcarbamoyl; 5- or 6-membered cyclic aminocarbonyl, optionally containing at least one further heteroatom in the ring, and wherein the ring is optionally substituted with at least one C1-C6 alkyl; 5-or-6-membered carbocyclylamino or heterocyclylamino; and 5-or-6-membered carbocyclyloxy or heterocyclyloxy; wherein any alkyl is optionally substituted with at least one halogen and any 5- or 6-membered carbocyclyl or heterocyclyl is optionally substituted with at least one R⁸;
each R⁷ and R⁸ is independently selected from C1-C6 alkyl; hydroxy-C0-C3 alkyl; C1-C6 alkoxy-C0-C3 alkyl; C1-C6 alkoxycarbonyl; carbocyclyl-C0-C4 alkyl; heterocyclyl-C0-C4 alkyl; C1-C6 alkylsulfinyl; amino; nitro; C1-C6 secondary or tertiary amino; halogen; carbamoyl; secondary or tertiary C1-C6 alkylamido-C0-C3 alkyl; C1-C6 alkylcarbonylamino; and 5- or 6-membered cyclic amino, optionally containing at least one further heteroatom in the ring and wherein the ring is optionally substituted with at least one C1-C6 alkyl; wherein any alkyl is optionally substituted with at least one halogen; wherein any carbocyclyl and heterocyclyl is 5- or 6-membered;
or a pharmaceutically acceptable salt thereof;
Item 2 The compound of item 1, wherein R1 is selected from H and C1-C3 alkyl;
Item 3 compound of item 1, wherein each R4 is H;
Item 4 The compound of item 1, wherein R3 is selected from H; halogen; and C1-C6 alkyl, wherein any alkyl is optionally substituted with at least one halogen;
Item 5 The compound of item 1, wherein n is 0;
Item 6 The compound of item 1, wherein Ra is H;
Item 7 The compound of item 1, wherein the compound is of formula (ICa) 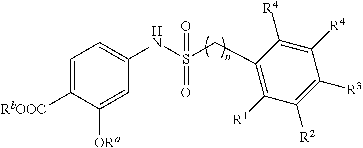
wherein R1, R4, Ra, Rb and n are as defined in item 1,
R2 is phenyl substituted with at least one R5, and R3 is H;
Item 8 The compound of item 1, wherein R2 is phenyl substituted with 1 or 2 moieties R5; and each R5 is independently selected from hydroxy, C1-C3 alkoxy and halogen;
Item 9 The compound of item 1, wherein R2 is 5-fluoro-2-hydroxyphenyl;
Item 10 The compound of item 1, wherein Rb is C2-C4 alkyl, substituted by 1 or 2 moieties selected from methoxy and ethoxy;
Item 11 The compound of item 1, wherein Rb is hydroxy-C2-C4 alkyl;
Item 12 The compound of item 1, wherein Rb is tetrahydrofuryl-C1-C0 alkyl;
Item 13 The compound of item 1, wherein compound is selected from the group consisting of
methyl 2-hydroxy-4-1[(4-methyl-1-naphthyl)sulfonyl]amino}benzoate,
methyl 2-hydroxy-4-[(1-naphthylsulfonyl)amino]benzoate,
methyl 4-{1[(4-fluoro-1-naphthyl)sulfonyl]amino}-2-hydroxybenzoate,
methyl 4-[(2,1,3-benzothiadiazol-4-ylsulfonyl)amino]-2-hydroxybenzoate,
methyl 2-hydroxy-4-[(naphthalen-2-ylsulfonyl)amino]benzoate,
methyl 4-({[5-(dimethylamino)naphthalen-1-yl]sulfonyl}amino)-2-hydroxybenzoate,
methyl 2-hydroxy-4-1[(2′-hydroxybiphenyl-3-yl)sulfonyl]amino}benzoate,
methyl 4-1[(3′-chlorobiphenyl-3-yl)sulfonyl]amino}-2-hydroxybenzoate,
methyl 4-[(biphenyl-3-ylsulfonyl)amino]-2-hydroxybenzoate,
methyl 2-hydroxy-4-1[(3-pyridin-3-ylphenyl)sulfonyl]amino}benzoate,
methyl 2-hydroxy-4-1[(3-pyridin-4-ylphenyl)sulfonyl]amino}benzoate,
methyl 4-({[3-(1-benzofuran-2-yl)phenyl]sulfonyl}amino)-2-hydroxybenzoate,
methyl 2-hydroxy-4-1[(3-quinolin-6-ylphenyl)sulfonyl]amino}benzoate,
methyl 4-{[(3′-aminobiphenyl-3-yl)sulfonyl]amino}-2-hydroxybenzoate,
methyl 4-{[(3′-acetamidobiphenyl-3-yl)sulfonyl]amino}-2-hydroxybenzoate,
methyl 2-hydroxy-4-1[(2′-nitrobiphenyl-3-yl)sulfonyl]amino}benzoate,
methyl 4-({[3-(5-acetyl-2-thienyl)phenyl]sulfonyl}amino)-2-hydroxybenzoate,
methyl 2-hydroxy-4-({[2′-(hydroxymethyl)biphenyl-3-yl]sulfonyl}amino)benzoate,
methyl 4-{[(3′-cyanobiphenyl-3-yl)sulfonyl]amino}-2-hydroxybenzoate,
methyl 2-hydroxy-4-({[4′-(methylsulfanyl)biphenyl-3-yl]sulfonyl}amino)benzoate,
methyl 2-hydroxy-4-({[4′-(trifluoromethoxy)biphenyl-3-yl]sulfonyl}amino)benzoate,
methyl 2-hydroxy-4-({[4′-(trifluoromethyl)biphenyl-3-yl]sulfonyl}amino)benzoate,
methyl 4-({[4′-(dimethylcarbamoyl)biphenyl-3-yl]sulfonyl}amino)-2-hydroxybenzoate,
methyl 4-{[(4′-carbamoylbiphenyl-3-yl)sulfonyl]amino}-2-hydroxybenzoate,
methyl 2-hydroxy-4-({[3′-(methylsulfonyl)biphenyl-3-yl]sulfonyl}amino)benzoate,
methyl 4-{[(3′-carbamoylbiphenyl-3-yl)sulfonyl]amino}-2-hydroxybenzoate,
methyl 2-hydroxy-4-({[5-(trifluoromethyl)biphenyl-3-yl]sulfonyl}amino)benzoate,
methyl 4-({[2′,5′-difluoro-5-(trifluoromethyl)biphenyl-3-yl]sulfonyl}amino)-2-hydroxybenzoate,
methyl 4-({[3-(2,3-dihydro-1-benzofuran-5-yl)-5-(trifluoromethyl)phenyl]sulfonyl}amino)-2-hydroxybenzoate,
methyl 2-hydroxy-4-({[2′-hydroxy-5-(trifluoromethyl)biphenyl-3-yl]sulfonyl}amino)benzoate,
methyl 4-({[3-(2,3-dihydro-1-benzofuran-5-yl)benzyl]sulfonyl}amino)-2-hydroxybenzoate,
methyl 4-{[(3′-ethoxybiphenyl-3-yl)sulfonyl]amino}-2-hydroxybenzoate,
1-methylethyl 3′-{[3-hydroxy-4-(methoxycarbonyl)phenyl]sulfamoyl}biphenyl-3-carboxylate,
methyl 4-{[(5′-fluoro-2′-hydroxybiphenyl-3-yl)sulfonyl]amino}-2-hydroxybenzoate,
benzyl 2-acetoxy-4-[(1-naphthylsulfonyl)amino]benzoate,
2-acetoxy-4-[(1-naphthylsulfonyl)amino]benzoic acid,
methyl 2-hydroxy-4-({[3-(piperidin-1-yl)phenyl]sulfonyl}amino)benzoate,
4-(dimethylamino)butyl 2-hydroxy-4-({[3-(2-methyl-1,3-thiazol-4-yl)phenyl]sulfonyl}amino)benzoate,
methyl 4-({[5′-fluoro-2′-hydroxy-5-(trifluoromethyl)biphenyl-3-yl]sulfonyl}amino)-2-hydroxybenzoate,
methyl 4-{[(2′,5′-difluorobiphenyl-3-yl)sulfonyl]amino}-2-hydroxybenzoate,
methyl 4-({[3-(2,3-dihydro-1-benzofuran-5-yl)phenyl]sulfonyl}amino)-2-hydroxybenzoate,
3-morpholin-4-ylpropyl 2-hydroxy-4-1[(2′-hydroxybiphenyl-3-yl)sulfonyl]amino}benzoate,
3-morpholin-4-ylpropyl 4-{[(5′-fluoro-2′-hydroxybiphenyl-3-yl)sulfonyl]amino}-2-hydroxybenzoate,
4-morpholin-4-ylbutyl 4-{[(5′-fluoro-2′-hydroxybiphenyl-3-yl)sulfonyl]amino}-2-hydroxybenzoate,
3-morpholin-4-ylpropyl 4-{[(2′,5′-difluorobiphenyl-3-yl)sulfonyl]amino}-2-hydroxybenzoate,
2-methoxyethyl 4-{[(2′,5′-difluorobiphenyl-3-yl)sulfonyl]amino}-2-hydroxybenzoate,
4-morpholin-4-ylbutyl 4-{[(2′,5′-difluorobiphenyl-3-yl)sulfonyl]amino}-2-hydroxybenzoate,
3-morpholin-4-ylpropyl 4-({[3-(2,3-dihydro-1-benzofuran-5-yl)phenyl]sulfonyl}amino)-2-hydroxybenzoate,
2-methoxyethyl 4-({[3-(2,3-dihydro-1-benzofuran-5-yl)phenyl]sulfonyl}amino)-2-hydroxybenzoate,
4-morpholin-4-ylbutyl 4-({[3-(2,3-dihydro-1-benzofuran-5-yl)phenyl]sulfonyl}amino)-2-hydroxybenzoate,
2-methoxy-1-methylethyl 4-{[(5′-fluoro-2′-hydroxybiphenyl-3-yl)sulfonyl]amino}-2-hydroxybenzoate,
tetrahydrofuran-3-yl 4-{[(5′-fluoro-2′-hydroxybiphenyl-3-yl)sulfonyl]amino}-2-hydroxybenzoate,
1-(methoxymethyl)propyl 4-{[(5′-fluoro-2′-hydroxybiphenyl-3-yl)sulfonyl]amino}-2-hydroxybenzoate,
2-ethoxy-1-(ethoxymethyl)ethyl 4-{[(5′-fluoro-2′-hydroxybiphenyl-3-yl)sulfonyl]amino}-2-hydroxybenzoate,
2-methoxybutyl 4-{[(5′-fluoro-2′-hydroxybiphenyl-3-yl)sulfonyl]amino}-2-hydroxybenzoate,
2-hydroxyethyl 4-{[(5′-fluoro-2′-hydroxybiphenyl-3-yl)sulfonyl]amino}-2-hydroxybenzoate,
3-hydroxypropyl 4-{[(5′-fluoro-2′-hydroxybiphenyl-3-yl)sulfonyl]amino}-2-hydroxybenzoate,
2-methoxyethyl 4-{[(5′-fluoro-2′-hydroxybiphenyl-3-yl)sulfonyl]amino}-2-hydroxybenzoate,
2-phenoxyethyl 4-{[(5′-fluoro-2′-hydroxybiphenyl-3-yl)sulfonyl]amino}-2-hydroxybenzoate,
3-(2,6-dimethylmorpholin-4-yl)propyl 4-{[(5′-fluoro-2′-hydroxybiphenyl-3-yl)sulfonyl]amino}-2-hydroxybenzoate,
3-(pyridin-3-ylamino)propyl 4-{[(5′-fluoro-2′-hydroxybiphenyl-3-yl)sulfonyl]amino}-2-hydroxybenzoate,
3-[(1-methyl-1H-pyrazol-5-yl)amino]propyl 4-{[(5′-fluoro-2′-hydroxybiphenyl-3-yl)sulfonyl]amino}-2-hydroxybenzoate,
3-[(5-methylisoxazol-3-yl)amino]propyl 4-{[(5′-fluoro-2′-hydroxybiphenyl-3-yl)sulfonyl]amino}-2-hydroxybenzoate,
or a pharmaceutically acceptable salt thereof;
Item 14 4-{[(5′-Fluoro-2′-hydroxybiphenyl-3-yl)sulfonyl] amino}-2-hydroxybenzoicacid or a pharmaceutically acceptable salt thereof.

37. The method of claim 15, comprising an administration by the subject in need of a compound with a specificity for a PFKFB3 polynucleotide selected from the group consisting of: PFKFB3 silencing therapy, an artificial microRNA, ribozyme, an antisense polynucleotide or a small interfering RNA (siRNA), a double stranded RNA, a short hairpin RNA.
